SEQUENCE LISTING (10 sequences)

SEQ ID NO: 1 - DNA, 1224 bp, Homo sapiens
atgcgctgggtgtgggcgctgctgaagaatgcgtccctggcaggggcgcccaagtacatagagcacttcagcaagttctccccgtccccgctgtccatgaagcagtttctggacttcggatccagcaatgcctgtgagaaaacctccttcaccttcctcaggcaggagctgcctgtgcgcctggccaacatcatgaaagagatcaacctgcttcccgaccgagtgctgagcacaccctctgtgcagctggtgcagagctggtatgtccagagcctcctggacatcatggagttcctggacaaggatcccgaggaccatcgcaccctgagccagttcactgacgccctggtcaccatccggaaccggcacaacgacgtggtgcccaccatggcacaaggcgtgcttgagtacaaggacacctacggcgatgaccccgtctccaaccagaacatccagtacttcctggaccgcttctacctcagccgcatctccatccgcatgctcatcaaccagcacaccctcatctttgatggcagcaccaacccagcccatcccaaacacatcggcagcatcgaccccaactgcaacgtctctgaggtggtcaaagatgcctacgacatggctaagctcctgtgtgacaagtattacatggcctcacctgacctggagatccaggagatcaatgcagccaactccaaacagccgattcacatggtctacgtcccctcccacctctaccacatgctctttgagctcttcaagaatgccatgagggcgactgtggaaagccatgagtccagcctcattctcccacccatcaaggtcatggtggccttgggtgaggaagatctgtccatcaagatgagtgaccgaggtgggggtgttcccttgaggaagattgagcgactcttcagctacatgtactccacagcacccaccccccagcctggcaccgggggaacgccgctggctggctttggttatgggctccccatttcccgcctctacgccaagtacttccagggagacctgcagctcttctccatggaaggctttgggaccgatgctgtcatctatctcaaggccctgtccacggactcggtggagcgcctgcctgtctacaacaagtcagcctggcgccactaccagaccatccaggaggccggcgactggtgtgtgcccagcacggagcccaagaacacgtccacgtaccgcgtcacgtaa

SEQ ID NO: 2 - protein, 407 aa, Homo sapiens
MRWVWALLKNASLAGAPKYIEHFSKFSPSPLSMKQFLDFGSSNACEKTSFTFLRQELPVRLANIMKEINLLPDRVLSTPSVQLVQSWYVQSLLDIMEFLDKDPEDHRTLSQFTDALVTIRNRHNDVVPTMAQGVLEYKDTYGDDPVSNQNIQYFLDRFYLSRISIRMLINQHTLIFDGSTNPAHPKHIGSIDPNCNVSEVVKDAYDMAKLLCDKYYMASPDLEIQEINAANSKQPIHMVYVPSHLYHMLFELFKNAMRATVESHESSLILPPIKVMVALGEEDLSIKMSDRGGGVPLRKIERLFSYMYSTAPTPQPGTGGTPLAGFGYGLPISRLYAKYFQGDLQLFSMEGFGTDAVIYLKALSTDSVERLPVYNKSAWRHYQTIQEAGDWCVPSTEPKNTSTYRVT

SEQ ID NO: 3 - DNA, 1224 bp, Homo sapiens
atgcatcatcatcatcatcaccttctggttccgcgtggatctgcgcccaagtacatagagcacttcagcaagttctccccgtccccgctgtccatgaagcagtttctggacttcggatccagcaatgcctgtgagaaaacctccttcaccttcctcaggcaggagctgcctgtgcgcctggccaacatcatgaaagagatcaacctgcttcccgaccgagtgctgagcacaccctctgtgcagctggtgcagagctggtatgtccagagcctcctggacatcatggagttcctggacaaggatcccgaggaccatcgcaccctgagccagttcactgacgccctggtcaccatccggaaccggcacaacgacgtggtgcccaccatggcacaaggcgtgcttgagtacaaggacacctacggcgatgaccccgtctccaaccagaacatccagtacttcctggaccgcttctacctcagccgcatctccatccgcatgctcatcaaccagcacaccctcatctttgatggcagcaccaacccagcccatcccaaacacatcggcagcatcgaccccaactgcaacgtctctgaggtggtcaaagatgcctacgacatggctaagctcctgtgtgacaagtattacatggcctcacctgacctggagatccaggagatcaatgcagccaactccaaacagccgattcacatggtctacgtcccctcccacctctaccacatgctctttgagctcttcaagaatgccatgagggcgactgtggaaagccatgagtccagcctcattctcccacccatcaaggtcatggtggccttgggtgaggaagatctgtccatcaagatgagtgaccgaggtgggggtgttcccttgaggaagattgagcgactcttcagctacatgtactccacagcacccaccccccagcctggcaccgggggaacgccgctggctggctttggttatgggctccccatttcccgcctctacgccaagtacttccagggagacctgcagctcttctccatggaaggctttgggaccgatgctgtcatctatctcaaggccctgtccacggactcggtggagcgcctgcctgtctacaacaagtcagcctggcgccactaccagaccatccaggaggccggcgactggtgtgtgcccagcacggagcccaagaacacgtccacgtaccgcgtcacgtgatag

SEQ ID NO: 4 - protein, 406 aa, Homo sapiens
  Hexa-histidine tag
MHHHHHHLLVPRGSAPKYIEHFSKFSPSPLSMKQFLDFGSSNACEKTSFTFLRQELPVRLANIMKEINLLPDRVLSTPSVQLVQSWYVQSLLDIMEFLDKDPEDHRTLSQFTDALVTIRNRHNDVVPTMAQGVLEYKDTYGDDPVSNQNIQYFLDRFYLSRISIRMLINQHTLIFDGSTNPAHPKHIGSIDPNCNVSEVVKDAYDMAKLLCDKYYMASPDLEIQEINAANSKQPIHMVYVPSHLYHMLFELFKNAMRATVESHESSLILPPIKVMVALGEEDLSIKMSDRGGGVPLRKIERLFSYMYSTAPTPQPGTGGTPLAGFGYGLPISRLYAKYFQGDLQLFSMEGFGTDAVIYLKALSTDSVERLPVYNKSAWRHYQTIQEAGDWCVPSTEPKNTSTYRVT

SEQ ID NO: 5 - protein, 394 aa, Homo sapiens
GSAPKYIEHFSKFSPSPLSMKQFLDFGSSNACEKTSFTFLRQELPVRLANIMKEINLLPDRVLSTPSVQLVQSWYVQSLLDIMEFLDKDPEDHRTLSQFTDALVTIRNRHNDVVPTMAQGVLEYKDTYGDDPVSNQNIQYFLDRFYLSRISIRMLINQHTLIFDGSTNPAHPKHIGSIDPNCNVSEVVKDAYDMAKLLCDKYYMASPDLEIQEINAANSKQPIHMVYVPSHLYHMLFELFKNAMRATVESHESSLILPPIKVMVALGEEDLSIKMSDRGGGVPLRKIERLFSYMYSTAPTPQPGTGGTPLAGFGYGLPISRLYAKYFQGDLQLFSMEGFGTDAVIYLKALSTDSVERLPVYNKSAWRHYQTIQEAGDWCVPSTEPKNTSTYRVT

SEQ ID NO: 6 - DNA, 75 bp, Homo sapiens
cgtgaattcatgcatcatcatcatcatcaccttctggttccgcgtggatctgcgcccaagtacatagagcacttc

SEQ ID NO: 7 - DNA, 35 bp, Homo sapiens
cgttctagactatcacgtgacgcggtacgtcgacg

SEQ ID NO: 8 - DNA, 1188 bp, Homo sapiens
ggatctgcgcccaagtacatagagcacttcagcaagttctccccgtccccgctgtccatgaagcagtttctggacttcggatccagcaatgcctgtgagaaaacctccttcaccttcctcaggcaggagctgcctgtgcgcctggccaacatcatgaaagagatcaacctgcttcccgaccgagtgctgagcacaccctctgtgcagctggtgcagagctggtatgtccagagcctcctggacatcatggagttcctggacaaggatcccgaggaccatcgcaccctgagccagttcactgacgccctggtcaccatccggaaccggcacaacgacgtggtgcccaccatggcacaaggcgtgcttgagtacaaggacacctacggcgatgaccccgtctccaaccagaacatccagtacttcctggaccgcttctacctcagccgcatctccatccgcatgctcatcaaccagcacaccctcatctttgatggcagcaccaacccagcccatcccaaacacatcggcagcatcgaccccaactgcaacgtctctgaggtggtcaaagatgcctacgacatggctaagctcctgtgtgacaagtattacatggcctcacctgacctggagatccaggagatcaatgcagccaactccaaacagccgattcacatggtctacgtcccctcccacctctaccacatgctctttgagctcttcaagaatgccatgagggcgactgtggaaagccatgagtccagcctcattctcccacccatcaaggtcatggtggccttgggtgaggaagatctgtccatcaagatgagtgaccgaggtgggggtgttcccttgaggaagattgagcgactcttcagctacatgtactccacagcacccaccccccagcctggcaccgggggaacgccgctggctggctttggttatgggctccccatttcccgcctctacgccaagtacttccagggagacctgcagctcttctccatggaaggctttgggaccgatgctgtcatctatctcaaggccctgtccacggactcggtggagcgcctgcctgtctacaacaagtcagcctggcgccactaccagaccatccaggaggccggcgactggtgtgtgcccagcacggagcccaagaacacgtccacgtaccgcgtcacgtgatag

SEQ ID NO: 9 - DNA, 1182 bp, Homo sapiens
gcgcccaagtacatagagcacttcagcaagttctccccgtccccgctgtccatgaagcagtttctggacttcggatccagcaatgcctgtgagaaaacctccttcaccttcctcaggcaggagctgcctgtgcgcctggccaacatcatgaaagagatcaacctgcttcccgaccgagtgctgagcacaccctctgtgcagctggtgcagagctggtatgtccagagcctcctggacatcatggagttcctggacaaggatcccgaggaccatcgcaccctgagccagttcactgacgccctggtcaccatccggaaccggcacaacgacgtggtgcccaccatggcacaaggcgtgcttgagtacaaggacacctacggcgatgaccccgtctccaaccagaacatccagtacttcctggaccgcttctacctcagccgcatctccatccgcatgctcatcaaccagcacaccctcatctttgatggcagcaccaacccagcccatcccaaacacatcggcagcatcgaccccaactgcaacgtctctgaggtggtcaaagatgcctacgacatggctaagctcctgtgtgacaagtattacatggcctcacctgacctggagatccaggagatcaatgcagccaactccaaacagccgattcacatggtctacgtcccctcccacctctaccacatgctctttgagctcttcaagaatgccatgagggcgactgtggaaagccatgagtccagcctcattctcccacccatcaaggtcatggtggccttgggtgaggaagatctgtccatcaagatgagtgaccgaggtgggggtgttcccttgaggaagattgagcgactcttcagctacatgtactccacagcacccaccccccagcctggcaccgggggaacgccgctggctggctttggttatgggctccccatttcccgcctctacgccaagtacttccagggagacctgcagctcttctccatggaaggctttgggaccgatgctgtcatctatctcaaggccctgtccacggactcggtggagcgcctgcctgtctacaacaagtcagcctggcgccactaccagaccatccaggaggccggcgactggtgtgtgcccagcacggagcccaagaacacgtccacgtaccgcgtcacgtgatag

SEQ ID NO: 10 - protein, 392 aa, Homo sapiens
APKYIEHFSKFSPSPLSMKQFLDFGSSNACEKTSFTFLRQELPVRLANIMKEINLLPDRVLSTPSVQLVQSWYVQSLLDIMEFLDKDPEDHRTLSQFTDALVTIRNRHNDVVPTMAQGVLEYKDTYGDDPVSNQNIQYFLDRFYLSRISIRMLINQHTLIFDGSTNPAHPKHIGSIDPNCNVSEVVKDAYDMAKLLCDKYYMASPDLEIQEINAANSKQPIHMVYVPSHLYHMLFELFKNAMRATVESHESSLILPPIKVMVALGEEDLSIKMSDRGGGVPLRKIERLFSYMYSTAPTPQPGTGGTPLAGFGYGLPISRLYAKYFQGDLQLFSMEGFGTDAVIYLKALSTDSVERLPVYNKSAWRHYQTIQEAGDWCVPSTEPKNTSTYRVT

(19) United States
(12) Patent Application Publication
(10) Pub. No.: US 2004/0058425 A1
(43) Pub. Date: Mar. 25, 2004

(54) Crystal structure

(76) Inventors: Thorsten Reginald Knoechel, Sandwich (GB); Colin Mark Robinson, Sandwich (GB); Wendy Elaine Taylor, Sandwich (GB); Alexander Dunbar Tucker, Sandwich (GB)

Correspondence Address: Gregg C. Benson, Pfizer Inc., Patent Department, MS 4159, Eastern Point Road, Groton, CT, 06340, US

(21) Appl. No.: 10/117,532

(22) Filed: Apr. 5, 2002

Related U.S. Application Data
(60) Provisional application No. 60/289,552, filed on May 8, 2001.
(60) Provisional application No. 60/289,609, filed on May 8, 2001.

(30) Foreign Application Priority Data
Apr. 6, 2001 (GB) GB 0108736.0
Apr. 9, 2001 (GB) GB 0108867.3

(51) Int. Cl. C12N 9/12; G06F 19/00; G01N 33/48; G01N 33/50

(52) U.S. Cl. 435/194; 702/19

(57) ABSTRACT

The present invention relates to crystals of pyruvate dehydrogenase kinase (PDHK) wherein the PDHK protein includes PDHK-2 amino acid sequence as shown in SEQ ID NO: 2 but starting at amino acid position 16, or a homologue, fragment, variant or derivative thereof.). The invention also relates to high resolution three dimensional structures of PDHK in the presence and absence of physiological and synthetic ligands obtained by X-ray diffraction and use of the structures to identify, design or select compounds that bind to PDHK. The invention further relates to compounds identified, designed or selected using high resolution structures of PDHK. Also provided are nucleotide sequences used to obtain crystallisable PDHK protein.

The present invention relates to crystals of pyruvate dehydrogenase kinase (PDHK), and more particularly to high resolution structures of PDHK in the presence and absence of physiological and synthetic ligands obtained by X-ray diffraction. The invention further relates to the use of the three-dimensional structure so determined to identify, design or select compounds that bind to PDHK. The invention further relates to the DNA construct used to obtain a crystallisable PDHK protein.

Intermittent claudication and skeletal muscle fatigue are symptoms of peripheral vascular disease and other ischaemia-related diseases, e.g. chronic obstructive pulmonary disease, congestive heart failure and angina. In some cases, symptomatic relief can be achieved by oral administration of dichloroacetate (DCA) (Stacpoole, P. W. et al. (1989) Metabolism 38, 1124-1144; Bersin, R. M. et al. (1997) Am. Heart J. 134, 841-855; Henderson, G. N. et al. (1997) J. Clin. Pharmacol. 37, 416-425), a well established (Whitehouse, S. et al. (1974) Biochem. J. 141, 761-774), but toxic (Stacpoole, P. W. et al., (1998) Drug Metab. Rev. 30, 499-539; Stacpoole, P. W. et al. (1998) Environ. Health Perspect. 106 (Suppl. 4), 989-994) activator of pyruvate dehydrogenase (PDH).

PDH is a multi-enzyme complex (for a review see Mattevi, A. et al. (1992) Curr. Opin. Struct. Biol. 2, 877-887) that catalyses the decarboxylation of pyruvate with the formation of acetyl-CoA and NADH. The PDH complex consists of a pyruvate decarboxylase (E₁), a dihydrolipoyl transacetylase (E₂) and a dihydrolipoyl dehydrogenase (E₃) component. PDH is regulated by product inhibition and reversible phosphorylation through PDH kinase (PDHK) and PDH phosphatase (Linn, T. C. et al. (1969) Proc. Natl. Acad. Sci. USA 62, 234-241). DCA inhibits ATP-dependent phosphorylation by PDHK, which explains its effect as PDH activator (Pratt, M. L. & Roche, T. E. (1979) J. Biol. Chem. 254, 7191-7196). Phosphorylation of PDH is mediated by non-covalent binding of PDHK to the lipoyl domain of the dihydrolipoyl transacetylase (E₂L₂) component of the pyruvate dehydrogenase complex (Ravindran, S. et al. (1996) J. Biol. Chem. 271, 653-662; Jackson, J. C. et al. (1998) Biochem. J. 334, 703-711).

Activation of PDH under conditions of reduced oxygen availability would promote the rapid formation of acetyl-CoA from pyruvate for entry in the citric acid cycle. In addition it would reduce the rate of accumulation of lactic acid and inorganic phosphate, which are thought to be a major factor in causing muscle fatigue (Fitts, R. H. (1994) Physiol. Rev. 74, 49-94). Furthermore, it would increase the yield of ATP per molecule of oxygen consumed by shifting metabolism from fatty acid oxidation to carbohydrates as primary energy source.

Crystallisation of PDHK enzymes would enable determination of the three dimensional structure of the native enzyme. Furthermore, analysis of crystalline forms in the presence and absence of physiological and/or synthetic ligands would allow for identification of ligand binding and/or protein interaction sites of PDHK enzymes. Such information is useful for design of compounds to modulate PDHK activity and intervene with phosphorylation of PDH by PDH kinase.

Several approaches may be used to intervene with phosphorylation of PDH by PDH kinase, e.g.

Inhibition of the kinase activity by (a) competitive ligand binding to the ATP binding site or (b) via allosteric regulatory sites.

Blocking of the PDHK:E₂L₂ interaction by binding to the E₂L₂ binding site, or blocking other protein-protein interactions associated with and facilitating ATP dependent phosphorylation of the PDH complex by PDHK.

Interference with binding of the PDHK substrate, i.e. the E₁ component of the PDH complex.

Development of these approaches has been hindered because efforts to produce a crystalline form of PDHK, such as PDHK isozyme 2 (PDHK-2), in particular human PDHK-2 (hPDHK-2) have been unsuccessful.

The technical problem to be addressed by the invention is the provision of a crystalline form of PDHK, such as PDHK-2, in particular hPDHK-2.

This invention provides a PDHK crystal wherein the PDHK protein includes or consists of PDHK-2 amino acid sequence as shown in SEQ ID NO: 2 but starting at amino acid position 16 (i.e. the PDHK protein includes, or consists of the PDHK-2 amino acid sequence as shown in SEQ ID NO: 10), or a homologue, fragment, variant or derivative thereof.

The invention alleviates the problems in the art in by provision of crystalline forms of PDHK, such as PDHK-2, in particular hPDHK-2 in which a portion of the N-terminal amino acid sequence of the native sequence is absent.

The X-ray structure of engineered human PDHK isozyme 2 (PDHK-2) has been determined in the presence and absence of physiological and synthetic ligands. The structure is the first detailed three-dimensional description of this important regulatory enzyme. The protein consists of an N-terminal regulatory domain with a four-helix bundle topology, and a C-terminal catalytic domain, belonging to the GHKL superfamily (Dutta, R. & Inouye, M. (2000) Trends Biochem. Sci. 25, 24-28).

At least four distinct ligand binding sites of PDHK have been identified, comprising the ATP binding site on the catalytic domain, as well as the putative binding site of the lipoyl domain of the E₂ component (E₂L₂) of the PDH complex, an allosteric binding site and the DCA binding site, located on the regulatory domain. The structures of complexes of PDHK with ligands bound at the various sites are useful for structure based ligand design.

Recently the structure of PDHK-2 from Rattus novegicus (rPDHK-2) has been determined in the presence of ADP at 2.5 Å resolution (Steussy et al (2001), J. Biol. Chem., 276 (40), 37433-37450).

One embodiment of the invention is a crystal of pyruvate dehydrogenase kinase (PDHK), preferably a crystal of PDHK-2, preferably mammalian PDHK-2, even more preferably human PDHK-2, or homologues, fragments, variants, or derivatives thereof.

In an embodiment of the invention the PDHK-2 protein will have amino acid sequence as shown in SEQ ID NO: 2, preferably the PDHK protein includes or consists of PDHK-2 amino acid sequence as shown in SEQ ID NO: 2 but starting at amino acid position 16 (i.e. the PDHK protein includes, or consists of the PDHK-2 amino acid sequence as shown in SEQ ID NO: 10), or a homologue, fragment, variant or derivative thereof; preferably the PDHK-2 amino acid sequence is preceded by an amino acid sequence that does not correspond to the amino acid sequence at positions 1 to 15 of SEQ ID NO: 2; more preferably the PDHK-2 amino acid sequence is preceded by amino acid sequence that is an affinity purification tag and, or amino acid sequence that is a specific protein cleavage site; yet more preferably the PDHK-2 sequence is preceded by an amino acid sequence that is an affinity purification tag followed by a specific protein cleavage site. Preferably the affinity purification tag is a multiple histidine tag, most preferably a six Histidine (Hexa-His) tag. Preferably the specific protein cleavage site is a thrombin cleavage site. In an especially preferred embodiment the PDHK protein consists of PDHK amino acid sequence preceded by a Hexa-His tag followed by a thrombin cleavage site, more preferably the PDHK protein additionally has an N-terminal methionine residue. Even more preferred is a crystal where the PDHK-2 has a sequence as shown in SEQ ID NO: 4, herein referred to as hexa-His-PDHK-2*. In a preferred embodiment of the PDHK crystal, the PDHK protein consists of a PDHK-2 amino acid sequence preceded by one or two amino acid residues. A further preferred embodiment is a PDHK crystal wherein the PDHK protein includes or consists of a PDHK-2 amino acid sequence as shown in SEQ ID NO: 5, or a homologue, fragment, variant or derivative thereof; in a particularly preferred embodiment the PDHK-2 amino acid sequence consists of a PDHK-2 amino acid sequence as shown in SEQ ID NO: 5, this last embodiment is hereinafter referred to as PDHK-2*. In an embodiment of the invention PDHK-2* is provided by thrombin cleavage of the PDHK-2 amino acid sequence as shown in SEQ NO: 4.

The above mentioned crystal of PDHK-2* grown in 100 mM sodium acetate pH 5.2-5.8, 5-10% isopropanol, and 25-150 mM MgCl₂, preferably in 100 mM sodium acetate pH 5.6-5.8, 6-9% isopropanol, and 75-125 mM MgCl₂ is also an embodiment of this invention, as well as said crystal having a space group P6₄, or said crystal having unit cell dimensions of a=b=109 Å+/−3 Å, c=85 Å+/−3 Å.

Another embodiment of the invention is a crystal of hexa-His-PDHK-2*, wherein the crystal is monoclinic, or wherein said crystal is grown in 100 mM citrate pH 5.5-5.7, 15% PEG 4K, and between 200-400 mM ammonium acetate, or wherein said crystal has a space group C2, or said crystal which has unit cell dimensions of a=90 Å+/−3 Å, b=54 Å+/−3 Å, c=83 Å+/−3 Å, and β=106°+/−2°.

Further embodiments are crystals according to the invention with heavy atoms soaked in, preferably the heavy atoms being selected from a group consisting of mercury, iridium, and osmium.

A further embodiment of the invention is a crystal of PDHK-2, PDHK-2*, or hexa-His-PDHK-2* with a PDHK substrate, preferably ATP, or ADP, and/or a PDHK ligand soaked in. Preferably, the ligand is 4-{(2,5)-dimethyl-4-[3,3,3-trifluoro-2-hydroxy-2-methylpropanoyl]piperazinyl}carbonyl)benzonitrile, herein referred to as Compound 1, or N-{4-[(ethylanilino)sulfonyl]-2-methylphenyl}-3,3,3-trifluoro-2-hydroxy-2-methylpropanamide, herein referred to as Compound 2, or N-(2-aminoethyl)-2-{3-chloro-4-[(4-isopropylbenzyl)oxy]phenyl)acetamide, herein referred to as Compound 3, or DCA (dichloroacetate).

Another embodiment of the invention is a crystal of liganded PDHK, preferably liganded PDHK-2*; preferably said crystal is grown in 100 mM MES pH6, 10% isopropanol and 200 mM calcium acetate in the presence of a PDHK ligand. Preferably, said PDHK ligand is Compound 1.

Another embodiment of the invention is any crystal as mentioned above, wherein said crystal diffracts X-rays to 3.8 Å or better resolution, preferably to 3.2 Å or better resolution, preferably to 2.8 Å or better resolution, more preferably to 2.5 Å or better resolution, yet more preferably to 2.4 Å or better resolution, most preferably to 2.2 Å or better resolution.

A further embodiment of the invention is a crystal of PDHK-2*, having the atomic coordinates set out in Table 3, or a derivative set as expressed in any reference frame. The invention also includes:

(a) co-crystals of PDHK-2* with Compound 1, having the atomic coordinates set out in Table 4, or a derivative set as expressed in any reference frame,

(b) co-crystals of PDHK-2* with Compound 1, having ATP soaked in, having the atomic coordinates set out in Table 5, or a derivative set as expressed in any reference frame,

(c) crystals of PDHK-2* with Compound 2 soaked in, having the atomic coordinates set out in Table 6, or a derivative set as expressed in any reference frame,

(d) crystals of PDHK-2* with Compound 3 soaked in, having the atomic coordinates set out in Table 7, or a derivative set as expressed in any reference frame, or

(e) crystals of PDHK-2* with DCA and ADP soaked in, having the atomic coordinates set out in Table 8, or a derivative set as expressed in any reference frame.

Another aspect of the invention is the use of the atomic coordinates of a PDHK crystal according to the invention for deriving the three-dimensional structure of PDHK-2, and the use of said three-dimensional structure of PDHK-2 to computationally or otherwise evaluate the binding interactions of a chemical compound with PDHK-2, preferably with a binding site on PDHK-2, even more preferably with the ATP binding site on PDHK-2, in particular the ATP binding site which includes, but is not limited to, residues selected from Glu251, Leu252, Lys254, Asn255, Ala256, Arg258, Ala259, Met288, Ser289, Asp290, Gly292, Gly293, Gly294, Val295, Tle300, Leu303, Leu323, Ala324, Gly325, Phe326, Gly327, Tyr328, Gly329, Leu330, Pro331, Leu346, Ser348, Thr354, Asp355, Ala356. The invention also includes the use of said three-dimensional structure of PDHK-2 to computationally of otherwise evaluate the binding interactions of a compound with the putative E₂L₂ binding site on PDHK-2, in particular with the putative E₂L₂ binding site on PDHK-2 which includes, but is not limited to, residues selected from Leu31, Gln35, Phe36, Asp38, Phe39, Thr48, Ser49, Phe52, Leu53, Met167, Leu168, Gln171, His172, Ile175, Phe176. Furthermore the invention includes the use of the three-dimensional structure of PDHK-2 as mentioned above to computationally or otherwise evaluate the binding interactions of a compound with a binding site on PDHK-2 which includes, but is not limited to, residues selected from Leu71, Pro72, Arg74, Val75, Thr78, Ser80, Val81, Met130, Gly133, Val134, Glu136, Tyr137, Asp144, Val146, Ser147,

Asn150, Ile151, Phe154, Leu155, Tyr382. The invention also includes the use of a three dimensional structure of PDHK-2 to computationally or otherwise evaluate the binding interactions of a compound with a binding site on PDHK-2 which includes, but is not limited to, residues selected from Leu57, Leu61, Tyr88, Ser91, Ile95, Ile119, Arg120, His123, Ser161, Arg162, Ile165, Arg166, Ile169.

Another aspect of the invention is the use of the three-dimensional structure of PDHK-2 according to the invention to design a compound, in particular a PDHK ligand, preferably a PDHK inhibitor, capable of associating with a PDHK enzyme, preferably to design a compound capable of associating with a binding site on the enzyme, even more preferred to design a compound capable of associating with any one of the binding sites on the enzyme mentioned above; even more preferred to design a compound capable of associating with an ATP binding site of the enzyme, or with the putative E₂L₂ binding site on the enzyme, or with the third binding site described, or with the DCA binding site.

Another embodiment of the invention is the use of the three-dimensional structure of PDHK-2 as mentioned above to computationally or otherwise evaluate the binding interactions of a compound, preferably a PDHK ligand, even more preferably a PDHK inhibitor, to the dimerisation interface, as well as the use of said structure to design compounds that can interact with said dimerisation interface.

The invention also relates to compounds, preferably PDHK ligands, even more preferably PDHK inhibitors, thus evaluated or designed.

Another aspect of the invention is a method of selecting a PDHK-2 inhibitor compound from a group of potential PDHK-2 inhibitor compounds, comprising the following steps:

a) creating a three-dimensional representation of the structure of PDHK-2 as mentioned above, or preferably the structure of any of the binding sites on the enzyme;

b) displaying and superimposing a model of the potential PDHK-2 inhibitor compound on the model of the PDHK-2 structure or on the model of the binding site on the enzyme; and

c) assessing whether the model of the potential PDHK-2 inhibitor compound fits the model of the PDHK-2 structure or the model of the binding site on the enzyme.

Optionally, said method can further comprise the following steps:

d) incorporating the potential PDHK-2 inhibitor compound in a biological PDHK-2 activity assay; and

e) determining whether the potential PDHK-2 inhibitor compound inhibits PDHK-2 activity in this assay.

The invention also includes compounds, in particular PDHK-2 ligands, preferably PDHK-2 inhibitors, selected by the method described above.

Another aspect of the invention is a pharmaceutical composition comprising a compound, preferably a PDHK ligand, even more preferably a PDHK inhibitor, evaluated, designed or selected as described above.

A further embodiment of the invention is the use of a compound, preferably a PDHK ligand, even more preferably a PDHK inhibitor, evaluated, designed or selected as described above, in the manufacture of a medicament for the treatment of diseases, wherein the inhibition of PDHK is beneficial, preferably ischemia-related diseases such as congestive heart failure, peripheral vascular disease, chronic obstructive pulmonary disease, coronary artery disease, diabetes, ischemia, endotoxic shock, hemorrhagic shock, lactic acidosis, or cardiac insufficiency.

Another aspect of the invention is the use of the atomic coordinates of PDHK-2 as mentioned above, or portions thereof, to solve a crystal form of a mutant, homologue or co-complex of PDHK-2, for example by Molecular Replacement or Difference Fourier analysis.

A further aspect of the invention is the use of the structure of PDHK-2 to design mimics of other PDHK isozymes or variants thereof, e.g. single nucleotide polymorphisms (SNPs), particularly in the binding site regions identified; the structure can be used to design site-directed mutants, but also to computationally (or otherwise) assess the binding of PDHK ligands to such closely related enzymes.

Furthermore, the invention includes the use of the atomic coordinates of PDHK-2 as mentioned above, or portions thereof, to produce a model of the three-dimensional structure of related proteins or variants or portions thereof. Such related proteins include, but are not limited to, other PDHK isozymes, PDHK isozymes from different animals, from plants, bacteria, or fungi, branched-chain α-keto acid dehydrogenase kinase, and others.

A further embodiment of the invention is a nucleotide sequence comprising the sequence shown in SEQ ID NO: 3, allelic variants, or mutants thereof or a nucleotide sequence comprising a sequence encoding the peptide sequence shown in SEQ ID NO: 4, allelic variants or mutants thereof; or the sequence as shown in SEQ ID NO: 9, or allelic variants, or mutants thereof; or a nucleotide sequence encoding PDHK-2 amino acid sequence as shown in SEQ ID NO: 2, but starting at amino acid position 16, or allelic variants or mutants thereof; or the sequence as shown in SEQ ID NO: 8 or allelic variants or mutants thereof, or a sequence encoding the amino acid sequence shown in SEQ ID No: 5, or allelic variants or mutants thereof. (An allelic variant is an alternative form of the enzyme, with a difference in the nucleic acid sequence, and generally produce altered mRNAs and potentially altered proteins, whose structure or function may or may not be altered. The difference can be a single nucleotide, often referred to as single nucleotide polymorphism (SNP), but can also involve larger changes. By mutants we mean alternative forms of the enzyme deliberately produced by methods well known to a person skilled in the art, such as site-directed mutagenesis.) Preferably this nucleotide sequence is linked to a regulatory sequence, in particular a promoter sequence, suitable for-driving expression in the desired host. Preferably, said nucleotide sequence is incorporated into a recombinant baculovirus, and said host is an insect cell line.

The terms “homologue”, “fragment”, “variant” or “derivative” in relation to the crystallized PDHK sequence include any substitution of, variation of, modification of, replacement of, deletion of or addition of one or more amino acid residues from or to the sequence, providing the resultant polypeptide is capable of folding to a structure similar to that of PDHK, in particular similar to that of PDHK-2. The term “homologue” covers homology with respect to sequence and/or structure and/or function. With respect to sequence homology, in proteins according to the invention, there is at least 65%, preferably 80%, more preferably 90%, even more preferably 95%, most preferably 98%, identity to the protein sequence of human PDHK-2 (SEQ ID NO: 2), or a significant part thereof for example to hPDHK-2 amino acid sequence as shown in SEQ ID NO: 2, but starting at amino acid position 16. A person skilled in the art will be well aware of suitable computer programmes to use to assess such sequence homology, which include, but are not limited to, the BLAST package (Altschul, S. F. et al (1997) Nucl. Acids Res. 25, 3389-3402), FASTA or FASTP (Pearson, W. R. (1990) Methods Enzymol. 183, 63-98), or programmes included in the GCG package (University of Wisconsin; Devereux et al (1984) Nucl. Acids Res. 12, 387) such as GAP or BESTFIT.

| | |
|---|---|
| ADP | adenosine diphosphate |
| ATP | adenosine triphosphate |
| CoA | coenzyme A |
| DCA | dichloro acetate |
| DNA | deoxyribonucleic acid |
| EDTA | ethylenediamine tetraacetic acid |
| Hsp | heat shock protein |
| MES | 2-(N-morpholino) ethanesulphonic acid |
| MOI | multiplicity of infection |
| NAD | nicotinamide adenine dinucleotide |
| NADH | nicotinamide adenine dinucleotide, reduced form |
| NIH | National Institute of Health, USA |
| PCR | polymerase chain reaction |
| PDB | Protein Data Bank |
| PDH | pyruvate dehydrogenase |
| PDHK | PDH kinase |
| PDHK-1 | PDHK isozyme 1 |
| PDHK-2 | PDHK isozyme 2 |
| hPDHK-2 | PDHK isozyme 2 from Homo sapiens (Human)rPDHK-2 PDHK |
| | isozyme 2 from Rattus norvegicus (Rat) |
| hexa-His-PDHK-2* | Engineered version of human PDHK-2, suitable for |
| | crystallisation; the sequence is shown in SEQ ID NO: 4 |
| PDHK-2* | Engineered version of human PDHK-2, suitable for crystallisation; |
| | the sequence is shown in SEQ ID NO: 5; the protein can be |
| | prepared by thrombin cleavage of hexa-His-PDHK-2* |
| PDHK-3 | PDHK isozyme 3 |
| PDHK-4 | PDHK isozyme 4 |
| PEG | polyethylene glycol |
| rmsd | root mean square deviation |
| rpm | rotations per minute |
| SDS-PAGE | sodium dodecyl sulfate polyacrylamide gel electrophoresis |
| SNP | single nucleotide polymorphism |
| Compound 1 | 4-{(2,5)-dimethyl-4-[3,3,3-trifluoro-2-hydroxy-2- |
| | methylpropanoyl]piperazinyl}carbonyl)benzonitrile |
| Compound 2 | N-{4-[(ethylanilino)sulfonyl]-2-methylphenyl}- |
| | 3,3,3-trifluoro-2-hydroxy-2-methylpropanamide |
| Compound 3 | N-(2-aminoethyl)-2-{3-chloro-4-[(4- |
| | isopropylbenzyl)oxy]phenyl}acetamide |

### EXAMPLES

The invention will now be illustrated by the following non-limiting examples and accompanying figures, in which:

SEQ ID NO: 1 illustrates the human PDHK-2 cDNA coding sequence;

SEQ ID NO: 2 illustrates the human PDHK-2 protein sequence ;

SEQ ID NO: 3 illustrates the DNA sequence engineered to encode hexa;His-PDHK-2*.

SEQ ID NO: 4 illustrates the protein sequence of hexa-His-PDHK-2*.

SEQ ID NO: 5 illustrates the protein sequence of HPDHK-2*.

SEQ ID NO: 6 shows the sequence of the 5′ primer used to amplify the DNA sequence of SEQ ID NO: 3

SEQ ID NO: 7 shows the sequence of the 3′ primer sequence used to amplify the DNA sequence of SEQ ID NO: 3;

SEQ ID NO: 8 illustrates the DNA sequence of PDHK-2*

SEQ ID NO: 9 illustrates human PDHK-2 DNA sequence starting at a position corresponding to amino acid residue Ala16 in the full length hPDHK-2 sequence.

SEQ ID NO: 10 illustrates human PDHK-2 protein sequence starting at a position corresponding to amino acid residue Ala16 in the full length hPDHK sequence.

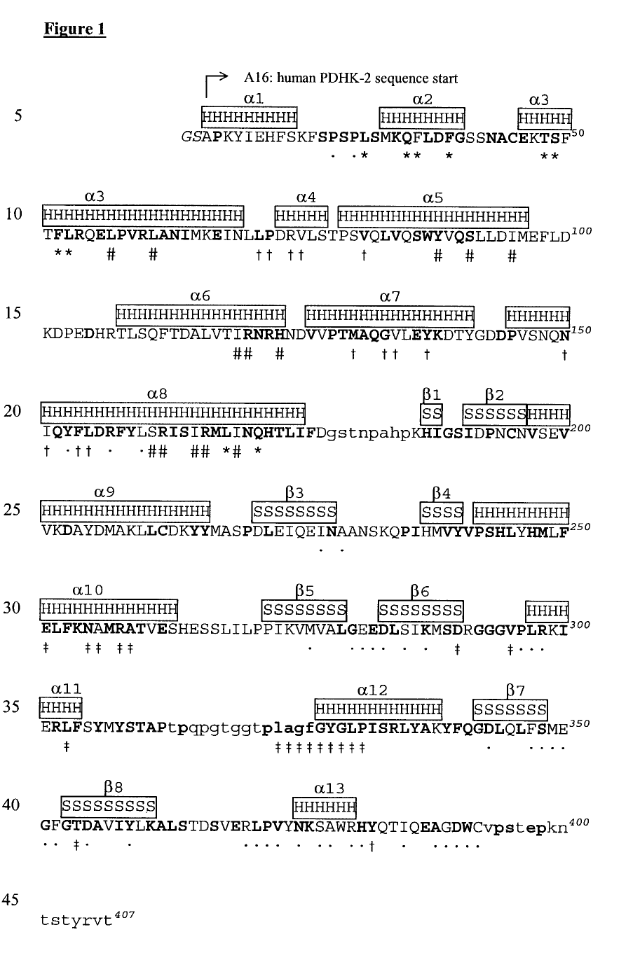

FIG. 1 shows the protein sequence of PDHK-2*, annotated to indicate the secondary structure features, residues interacting with ligands or located at the dimerisation interface, and conserved residues in all 4 human PDHK isozymes.

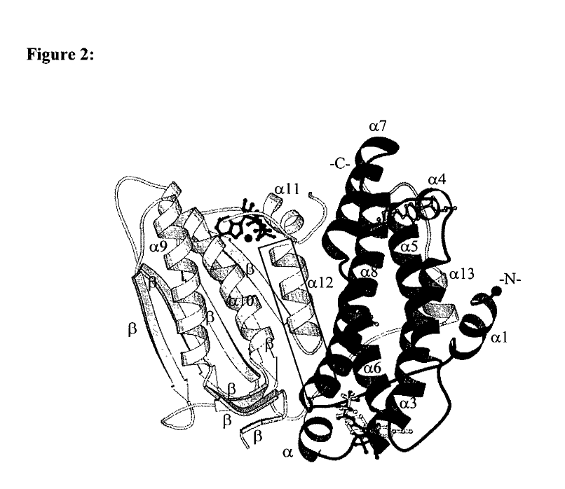

FIG. 2 shows a ribbon diagram of the PDHK-2 structure.

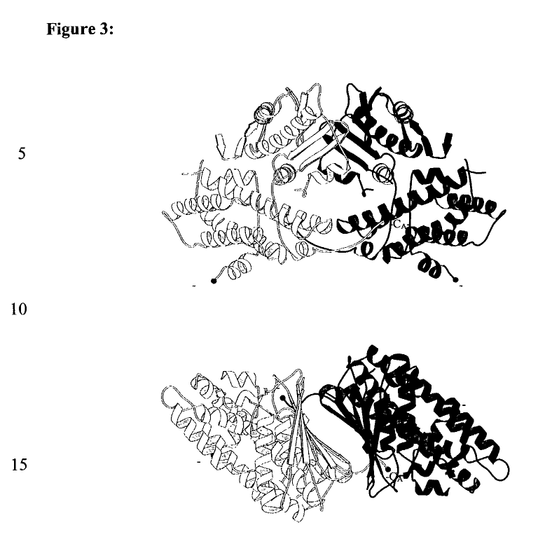

FIG. 3 shows two views of the PDHK-2 dimer.

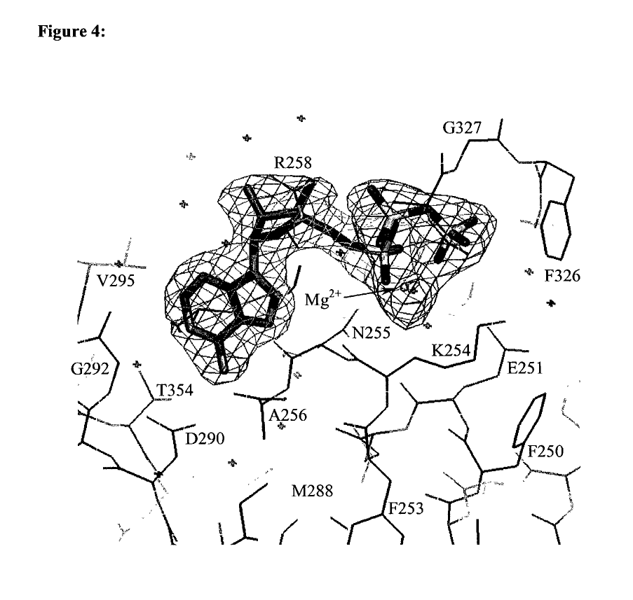

FIG. 4 shows ATP and Mg²⁺ together with the coordinating water molecules at the ATP binding site of PDHK-2.

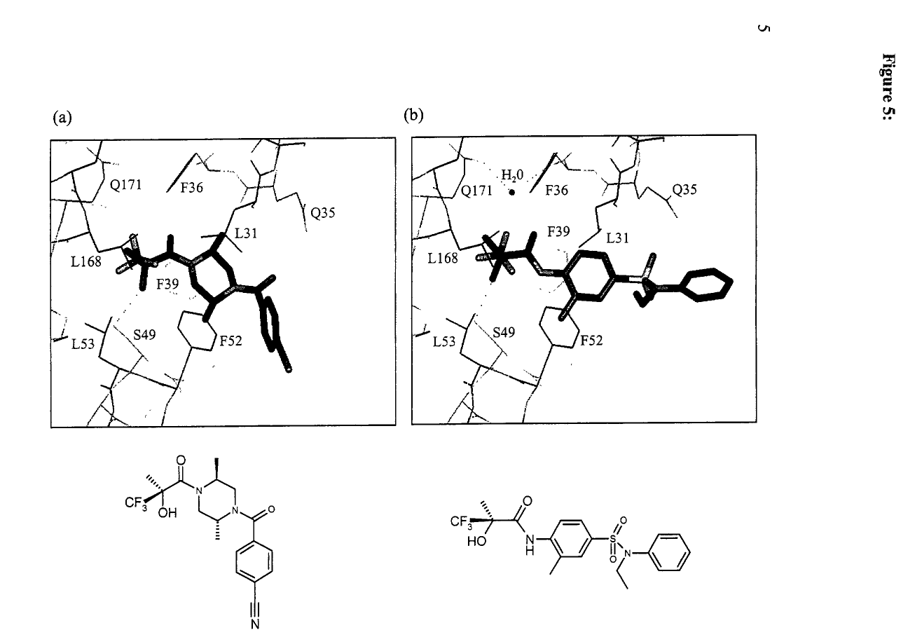

FIG. 5 shows views of (a) Compound 1 and (b) Compound 2 bound to PDHK-2, as well as their chemical structures depicted underneath.

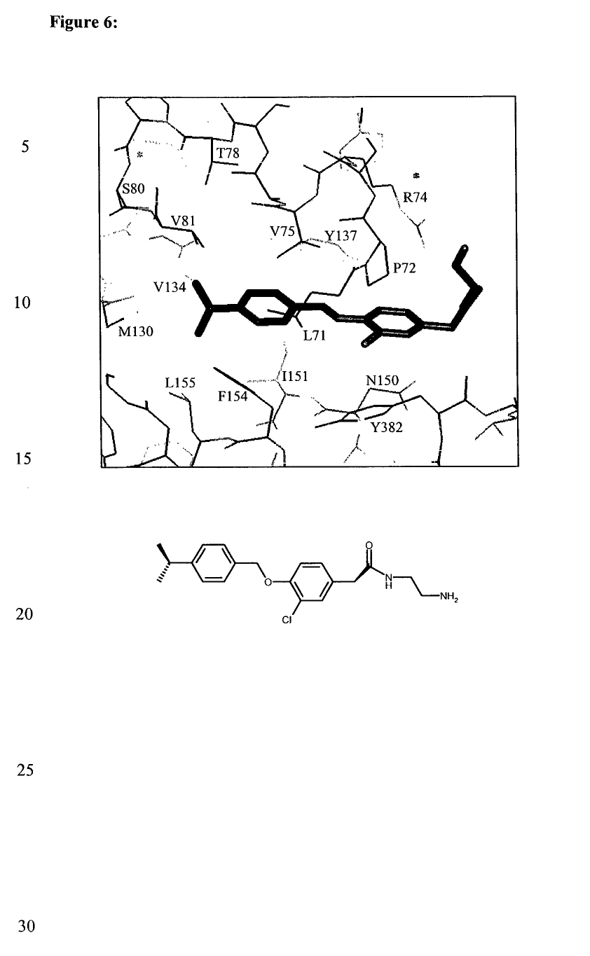

FIG. 6 shows a view of Compound 3 bound to PDHK-2, as well as its chemical structure.

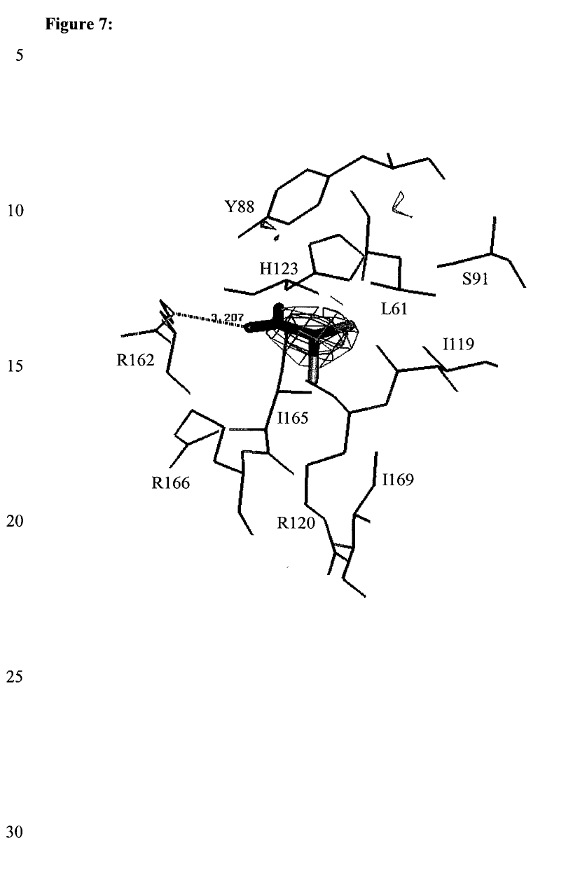

FIG. 7 shows the f_{obs} (ADP, DCA)-f_{obs} (native) electron density map with a model of DCA superimposed.

### Example 1

### Design of a Construct to Enable Crystallisation of Human PDHK-2

Attempts to crystallise full length human PDHK-2 were unsuccessful, thus steps were taken to engineer a construct to express a crystallisable form of human PDHK-2.

Subjecting human PDHK-2 to partial chymotrypsin digestion (1 hour at 30° C.) generated a predominant protein species with truncations on both ends. The N-terminal residue of the truncation product was determined to be Lys-14 by N-terminal sequencing; from the molecular weight it was deduced that 16 residues had been removed from the C-terminus as well. This truncated form of the enzyme retained enzymatic activity, therefore it was unlikely that the removed parts of the enzyme were essential for activity or the structural integrity of the enzyme.

A N-terminally truncated form of PDHK-2 was engineered, the expression construct comprising the DNA sequence encoding a methionine, a tag consisting of 6 histidine residues, followed by a Tobacco Etch Virus (TEV) protease cleavage site, followed by the human PDHK-2 sequence starting with residue Lys14. Following expression and purification of the resulting protein, crystallisation trials yielded only very small crystals; when subjected to MALDI-TOF mass spectrometry, two prominent protein species were detected, one of full length, and one truncated within the TEV protease cleavage site.

From secondary structure predictions, it appeared that the truncation at the N-terminus occurred near the start of a predicted helical region. The starting residue of this helix was predicted to be Ala16.

The implication of all the data mentioned above was that the N-terminal region of the protein may be rather flexible and potentially adversely affecting the packing of the crystal lattice, and that a more constrained part of the structure may start with the predicted helix starting at Ala16. It was therefore decided to concentrate on designing constructs starting with position Ala116 of the human PDHK-2 sequence. A hexa-histidine tag was included for ease of purification, and a thrombin cleavage site was engineered in to enable the tag to be removed during the purification and prior to crystallisation.

### Example 2

### Cloning and Expression of Crystallisable PDHK-2

Oligonucleotide primers were designed for use in polymerase chain reaction (PCR) amplification of the desired sequence, based on the sequence of human PDHK-2 (Gudi, R. et al (1995) J. Biol. Chem. 270, 28989-28994, GenBank Accession number L42451). The 5′ primer was designed to comprise DNA sequence encoding, following the ATG start codon, six histidine residues, followed by a thrombin cleavage site, and then linking in-frame with human PDHK-2 starting at residue Ala16. Both primers contained a restriction endonuclease site for ease of inserting the PCR product into the desired vector:

| | |
|---|---|
| 5′ primer: PDHK-2 Fast5′HisThr: | |
| 5′-CGTGAATTC ATG CAT CAT CAT CAT CAT CAC CTT CTG GTT CCG CGT | (SEQ ID NO:6) |
| GGA TCT GCG CCC AAG TAC ATA GAG CAC TTC-3′ | |
| 3′ primer: PDHK-2 Fast3′: | |
| CGTTCTAGA CTA TCA CGT GAC GCG GTA CGT CGA CG-3′ | (SEQ ID NO:7) |

DNA fragments were generated by PCR amplification from a clone containing full length human PDHK-2. In discrepancy to the published human PDHK-2 sequence as shown in GenBank accession L42451, apart from containing some silent single nucleotide differences, codon 80 encodes a serine instead of the threonine in the published sequence (SEQ ID NO: 1).

The PCR reaction was carried out for 30 cycles in a total volume of 50 μl in a solution containing 1.5 mM MgCl₂, 0.2 mM dNTPs, 50 pmol of each primer, 5 μl 10×Reaction Buffer (Roche Diagnostics), 0.2 μl template (1 ng) (a plasmid containing full length human PDHK-2 cDNA), and 2.5 units Expand DNA polymerase (Roche Diagnostics). Each cycle consisted of 1 minute at 94° C., 1 minute at 50° C., and 2 minutes at 72° C. Amplified DNA fragments were separated by agarose gel electrophoresis, and the desired PCR product purified using a QIA quick gel extraction kit (Qiagen). The purified PCR product was then digested with EcoR1 and Xba1, using Multicore buffer (Promega) at 37° C. for 2 hours. The digested fragment was then ligated into pFastbac1, also digested with EcoR1/Xba1 (Life Technologies), using DNA ligase (Promega) according to the manufacturer's instructions, for 12 hours at 12° C. The ligation mix was electroporated into E.coli (TOP 10) (Invitrogen), using a BioRad Gene Pulser (capacitance 25 μFD, voltage 2.5 V, resistance 200Ω).

Clones containing the desired insert were selected by using 2YT plates containing 100 μg/ml ampicillin and checked using endonuclease digestion, followed by agarose gel electrophoresis, for the presence of an insert of the correct size. DNA sequence analysis was carried out by Lark (Saffron Waldon, UK). The sequence of the DNA insert is shown as SEQ ID NO: 3.

Recombinant bacmid DNA was produced by transforming E.coli DH10BAC™ with the recombinant pFastbac1 plasmid DNA as described above. This was carried out according to the method in the Bac to Bac baculovirus expression manual (Life Technologies). PCR analysis was used to verify successful transposition to the bacmid using pUC/M13 amplification primers (Life Technologies).

Generation of the primary baculovirus stock was carried out by transfection using Sf-9 insect cells. Bacmid DNA containing the correct insert was mixed with CELLFECTIN™ transfection reagent (Life Technologies) and added to a monolayer of Sf-9 insect cells using SF-900II serum free medium (Life Technologies). Following 72 hours incubation at 27° C., the supernatant was harvested as the initial baculovirus stock. This stock was amplified by adding the initial baculovirus stock into a suspension of Sf-9 insect cells in Erlenmeyer flasks (Coming) with agitation of 125 rpm at 27° C. After 6 days post infection, the supernatant was harvested by centrifugation and stored at 4° C. as the working virus stock. The titre of this stock was determined by conventional plaque assay analysis as in the Bac to Bac baculovirus expression manual (Life Technologies).

Protein expression was optimised in Erlenmeyer flask cultures using Sf-9 and T.ni Hi-5 insect cell cultures looking at different multiplicities of infection (MOI) and harvest times, the optimal conditions found were then scaled up into fermenters.

The fermenters used were autoclavable Applikon 3 liter stirred vessels controlled using Applikon 1030 biocontrollers. Inoculum of T.ni Hi-5 cells was initially prepared as shake flask cultures. The fermenter was inoculated with 5×10⁵ cells/ml made up in Excel 405 serum free medium (JRH Biosciences). Temperature was controlled at 27° C., dissolved oxygen concentration controlled at 60%, and pH was measured but not controlled. Oxygen concentration was controlled by initially enriching the air stream to the headspace with oxygen, then sparging pure oxygen to the culture. Agitation was set at 150 rpm with a double impeller system of marine impeller within the culture and rushton impeller at the liquid/headspace interface. Aeration was continuous to the headspace at 0.5 l/min.

When the viable cell density reached 2×10⁶ cells/ml, the culture was infected using an MOI of 2 from the titred baculovirus working stock. Harvest time for the culture was 72 hours post infection. This was achieved by centrifugation at 2000 g for 15 mins; the insect cell pellet was then stored at −80° C. awaiting purification.

### Example 3

### Purification of hexa-His-PDHK-2* and Removal of the hexa-histidine Tag

PDHK-2*, having the sequence as shown in SEQ ID NO: 5, was purified in five steps: (1) Pellet from the fermentation was resuspended into 10 ml lysis buffer per gram wet cell weight and mechanically broken using a Constant Systems continuous cell disrupter at a pressure of 20 kpsi. The lysis buffer consisted of 50 mM Tris HCl (pH 8), 150 mM sodium chloride, 10 mM Imidazole, 5 mM β-mercaptoethanol, containing EDTA free protease inhibitor cocktail tablets (Roche). The lysate was chilled and centrifuged at 14,000 g for 45 mins to remove cell debris. (2) The supernatant was collected and applied to Nickel-NTA Superflow chelate (Qiagen), using 20 ml drip columns and 3 ml resin bed volume. The resin was initially washed with 10 column volumes lysis buffer and eluted with lysis buffer containing 100 mM Imidazole. (3) The eluted fraction was then buffer exchanged into Thrombin cleavage buffer (20 mM TrisHCl (pH8), 150 mM sodium chloride and 2.5 mM CaCl₂) using a 75 ml G-25 fine desalting column (Pharmacia) at a flow-rate of 7.5 ml/min. The desalted protein fractions were then pooled, and thrombin (Sigma T-1063) added to a final concentration of 5 NIH units/mg protein in solution. An incubation of 3 hours at room temperature then followed in order to allow for the removal of the hexa-histidine tag. (4) After thrombin cleavage the sample was applied to a 3 ml Nickel-NTA Superflow column (Qiagen) and the flow-through collected. The remainder of the cleaved PDHK-2* was removed from the column by elution with thrombin cleavage buffer containing 10 mM Imidazole. (5) The flow-through and the 10 mM imidazole fractions were pooled and concentrated to 2 mg/ml using 10 kDa centrifugal concentrators (Vivascience), and loaded at 0.75 ml/min onto a Sephacyl -200 prep grade 16/60 column pre-equilibrated with 20 mM TrisHCl (pH8), 150 mM sodium chloride and 1 mM DL-dithiothreitol on an Akta-Explorer purification system (Amersham Pharmacia Biotech). The eluted fractions were run on Tris-glycine SDS-PAGE gels and fractions containing PDHK-2* pooled. These were then stored at −80° C. prior to crystallisation.

### Example 4

### Purification of hexa-His-PDHK-2*

Hexa-His-PDHK-2*, i.e. the expression product of the construct produced in Example 2 having the sequence as shown in SEQ ID NO: 4, was purified by steps (1) and (2) as described in Example 3 above for the thrombin-cleaved material (PDHK-2*). Then the 100 mM imidazole fraction was pooled and concentrated to 2 mg/ml using 10 kDa centrifugal concentrators (Vivascience), and loaded at 0.75 ml/min onto a Sephacyl -200 prep grade 16/60 column pre-equilibrated with 20 mM TrisHCl (pH8), 150 mM sodium chloride and 1 mM DL-dithiothreitol on an Akta-Explorer purification system (Amersham Pharmacia Biotech). The eluted fractions were run on Tris-glycine SDS PAGE gels and fractions containing hexa-His-PDHK-2* were pooled. These were then stored at −80° C. prior to crystallisation.

### Example 5

### Crystallisation of PDHK-2*, Liganded PDHK-2*, and hexa-His-PDHK-2*

(a) Crystallisation of PDHK-2*

The PDHK-2* fractions from the final gel filtration column, obtained as described in Example 3, were concentrated to 10 mg/ml using 10 kDa centrifugal concentrators (Vivascience) at 3,000 rpm, 4° C. The crystallisation trials were set up using hanging drop vapour diffusion at 4° C. PDHK-2* crystallised in a range of conditions using 100 mM sodium acetate pH 5.2-5.8, 5-10% iso-propanol and 25-150 mM magnesium chloride. Prior to crystallisation the PDHK-2* at 10 mg/ml was centrifuged for 2 min at 14,000 rpm in an Eppendorf centrifuge. A reservoir volume of 950 μl was used, and drops of 2 μl reservoir reagent and 2 μl PDHK-2* suspended over the reservoir on cover slips. Larger crystals of PDHK-2* were obtained at pH 5.6-5.8 with 6-9% isopropanol and 75-125 mM magnesium chloride. These crystals belonged to space group P6₄ with cell dimensions a=b=109.3 Å, c=85.1 Å. They contained one molecule per asymmetric unit (M_{w}=44.6 kDa), and had a solvent content of 62% (V_{M}=3.29; Matthews, B. W. (1968) J. Mol. Biol. 33, 491-497). Crystals appeared overnight and continued to grow for up to 2 weeks.

(b) Co-crystallisation of PDHK-2* with Compound 1

For co-crystallisation studies PDHK-2* was mixed with Compound 1 (4-{(2,5)-dimethyl-4-[3,3,3-trifluoro-2-hydroxy-2-methylpropanoyl]piperazinyl}carbonyl)benzonitrile; Aicher. T. et al. (1999) J. Med. Chem. 42, 2741-2746) at 3-fold molar excess before getting concentrated to 10 mg/ml, and being used in crystallisation trials. This liganded PDHK-2* crystallised under similar conditions to unliganded PDHK-2*. The liganded PDHK-2* also yielded good crystals in 100 mM MES pH6, 10% isopropanol and 200 mM calcium acetate.

(c) Crystallisation of hexa-His-PDHK-2*

The stored hexa-His-PDHK-2* solution was concentrated to 10 mg/ml using 10 kDa centrifugal concentrators (Vivascience) at 3,000rpm, 4° C. Monoclinic crystals of hexa-His-PDHK-2* were grown by hanging drop vapour diffusion at 4° C., from 100 mM Citrate pH 5.6, 15% PEG 4K and between 200-400 mM ammonium acetate. The crystals grew as thin plates from a precipitate over a period of 2-3 weeks and belonged to space group C2 with unit cell dimensions a=90.8 Å, b=53.8 Å, c=83.3 Å, β=105.64°.

### Example 6

### Data Collection, Structure Determination and Refinement of PDHK-2*

The structure of recombinant human PDHK-2* was solved by multiple isomorphous replacement (MIR) using three heavy atom derivatives (Drenth, J. (1994) Principles of protein X-ray crystallography. Springer-Verlag, New York). Mercury, iridium and osmium heavy atom derivatives were prepared by soaking crystals for 16-18 hours in a stabilising solution (9% isopropanol in 100 mM sodium acetate buffer at pH 5.8) containing 1 mM ethylmercury thiosalicylate, 5 mM K₂IrCl₆, or 10 mM K₂OsCl₆, respectively.

For data collection protein crystals were flash-frozen at 100 K using a cryo-protection buffer consisting of stabilising solution and 30-35% glycerol. Native X-ray diffraction data, i.e. for PDHK-2* crystals without heavy atoms, were collected at 100 K with an ADSC Quantum 4 CCD-detector at station 9.6 (λ=0.8700 Å) of the S.R.S. Daresbury, UK. The diffraction data for the heavy atom derivatives were collected at 100 K with Rigaku RAXIS-2 or RAXIS-4 image plate detectors on laboratory rotating anode generators using CuKα radiation. All data were processed using the HKL package (Otwinowski, Z. & Minor,W. (1997) Methods Enzymol. 276, 307-326). Data collection statistics are summarised in Table 1 (see end of Example section).

Heavy atom derivative data were scaled to native data with SCALEIT (Collaborative Computational Project Number 4 (1994) Acta Crystallogr. D 50, 760-763: CCP4 suite). Difference Patterson and difference Fourier analysis identified initial heavy atom positions. Refinement of the heavy atom parameters and phase calculation was performed with SHARP (de La Fortelle, E. & Bricogne, G. (1997) Methods Enzymol. 276, 472-494). Phases were improved by 120 cycles of solvent flattening with SOLOMON (Abrahams, J. P. & Leslie, A. (1996) Acta Crystallogr. D 52, 30-42). The resulting map was of good quality and used to trace about 75% of the structure using QUANTA (Quanta98 (1998), version 98.1111; Molecular Simulations Inc., San Diego, Calif. 92121-3752).

The atomic coordinates are shown in Table 3 (see end of Example section).

The model was refined against a set of native structure factors (F_{P}-calc) derived with SHARP from a combination of experimental native (F_{P}) and derivative (F_{PH}) structure factors, with the respective heavy atom contributions (F_{H}) substracted. Refinement was carried out in the resolution range 30-2.2 Å using CNX (Brünger, A. et al. (1998), Acta Crystallogr. D 54, 905-921) with the “mlhl” maximum likelihood target function. During refinement model phases were combined with the experimental phase probability distributions provided by SHARP. Partial structure factors from a flat bulk-solvent model and anisotropic B-factor correction were supplied throughout the refinement. The R-factor for the current model is 0.267 (free R-factor, 5% of data, 0.297) for all data in the resolution range 30-2.2 Å. The refinement statistics are summarised in Table 2 (see end of Example section).

The current model contains 355 out of 394 residues present in the construct and is well defined in most regions of the polypeptide chain. The electron density is weak for residues 40-43, 73-79, 105-109, 310-312 and 375-392. No interpretable electron density was observed for residues 178-185, 313-326 and 393-407. The latter are not included in the model, and residues 386-387 are modelled as alanine.

### Example 7

### Preparation, Data Collection, Structure Determination and Refinement of PDHK-2* Protein/ligand complexes

Crystals of PDHK-2* in complex with Compound 1 were obtained by co-crystallisation as described in Example 5(b) above. Crystals of PDHK-2* in complex with Compound 2 (N-{4-[(ethylanilino)sulfonyl]-2-methylphenyl}-3,3,3-trifluoro-2-hydroxy-2-methylpropanamide; Example 12 in patent application WO 99/62873) and Compound 3 (N-(2-aminoethyl)-2-{3-chloro-4-[(4-isopropylbenzyl)oxy]phenyl}acetamide; see Example 10) were prepared by soaking apo-crystals, i.e. crystals prepared with unliganded PDHK-2* as described in Example 5(a) above, in stabilising solution containing the inhibitor at 1 mM concentration. Crystals of the PDHK-2* in complex with ATP were obtained by soaking crystals, grown in the presence of Compound 1, in stabilising solution containing 10 mM ATP. Crystals of the PDHK-2* in complex with ADP and DCA were obtained by soaking apo-crystals in stabilising solution containing both ADP and DCA at 10 mM and 100 mM concentration, respectively.

For data collection protein crystals were flash-frozen at 100 K using a cryo-protection buffer consisting of stabilising solution and 30-35% glycerol. Diffraction data of PDHK-2* in complex with Compound 1, Compound 2, and both ATP and compound 1 were collected at 100 K on an ADSC Quantum 4 CCD-detector at beamline ED14-2 (λ=0.9326 Å) of the European Synchiotron Radiation Facility (ESRF), Grenoble, France. Diffraction data of PDHK-2* in complex with Compound 3 were collected at 100 K on a Rigaku RAXIS-2 image plate detector with a laboratory rotating anode generator using CuKa radiation. Diffraction data of PDHK-2* in complex with both ADP and DCA were collected at 100K on an ADSC Quantum 4 CCD-detector at station 9.6 (λ=0.87 Å) of the Synchiotron Radiation Source (SRS), Daresbury, UK. Data processing was carried out with the HKL program package (Otwinowski, Z. & Minor, W. (1997) Methods Enzymol. 276, 307-326). Data collection statistics are summarised in Table 1 (see end of Example section).

The structures were determined by difference Fourier methods using the unliganded PDHK-2* structure to calculate model phases and subsequently refined using CNX (Brunger, A. et al. (1998), Acta Crystallogr. D 54, 905-921). Interactive graphical model building was carried out with QUANTA (Quanta98 (1998) version 98.1111; Molecular Simulations Inc., San Diego, Calif. 92121-3752). In all structures the respective ligands were clearly defined by the initial electron density maps. For a summary of refinement statistics see Table 2 (see end of Example section). A binding site of DCA on PDHK-2 has been identified by calculating a difference fourier map with [f_{obs} (ADP/DCA)-f_{obs} (native)] expiα_{calc} (native) as coefficients, where f_{obs} (ADP/DCA) are the observed structure factor amplitudes of PDHK-2* soaked with both ADP and DCA, f_{obs} (native) are the observed structure factor amplitudes of unliganded PDHK-2*, and α_{calc} (native) are the phases derived from the refined model of unliganded PDHK-2* (Example 6).

The atomic coordinates are given in Tables 4, 5, 6, 7 and 8. (see end of Example section).

### Example 8

### The Structure of PDHK-2

The structure of PDHK-2* is summarised in FIG. 1, which shows the peptide sequence of PDHK-2*, annotated with the observed secondary structure features (boxes with H indicating residues involved in an α-helix, boxes with S indicating residues involved in a β-strand), labelled as referred to in the text below. Residues in bold are those conserved in all four human PDHK isozymes (49% in total, 45% in the regulatory domain, 52% in the catalytic domain). Residues labelled with (*) are in direct contact (i.e. within a distance of 4 Å) with Compound 1; residues in direct contact with ATP are labelled with (‡); residues in direct contact with a compound bound to the third site identified (Compound 3) are labelled with (†); residues involved in the dimer interface are labelled with (•); residues forming the DCA binding pocket are labelled with (#). Residues shown in lower case are disordered in the structure of the native enzyme (Example 6). The sequence of the human PDHK-2 starts with residue Alal 6. The two preceding residues (Gly and Ser) arise from the engineered thrombin cleavage site, linking the PDHK-2 sequence to the N-terminal hexa-histidine tag

(a) Description of the Native Structure and Dimer Assembly

The structure of PDHK-2*, prepared as described in Example 3, crystallised as described in Example 5(a), solved as described in Example 6 above, can be described as follows:

PDHK-2 is composed of two domains (see FIG. 2). The Figure shows a ribbon diagram of the PDHK-2 structure. The N-terminal regulatory domain is shown in dark grey, the C-terminal catalytic domain in light grey. Ligands binding at various sites are shown as ball-and-stick models: ATP-Mg²⁺ in dark grey, Compound 1 in white, Compound 2 in black; Compound 3 in medium grey; DCA (hidden behind helix α6) in light grey. Termini and secondary structural elements are labelled. Helix α13 is hidden behind helices α3 and α5. The location of a groove between the regulatory and catalytic domain, comprising the potential binding site for a helix of the substrate protein, is indicated by a rectangle.

The structure of the N-terminal domain (residues 14-177) consists of eight helices with a distorted, antiparallel four-helix bundle core. The core is formed by helices α3, α5, α6+α7, and α8. The helices α1 and α2 precede the central four-helix bundle, whereas the short helix α4 connects helices α3 and α5. A topological comparison of the PDHK-2 N-terminal domain with the PDB does not reveal significant additional homology with other known protein structures, apart from the four-helix bundle.

The C-terminal domain of PDHK-2 (residues 186-407) consists of five helices and eight strands. It is connected to the regulatory domain by eight residues, which are disordered in the crystal structure. The central motif is an open, five-stranded, mixed β-sheet with a right handed twist of approximately 40°. It is formed by strands β3, β5, β6, β8 and β7 (directions ++−+−) and flanked on one side by a layer of helices (α9, α10, α11 and α12). On the same side but oriented perpendicular to the central β-sheet, strands β1, β2 and β4 form an additional, antiparallel β-sheet.

The C-terminal domain of PDHK-2 belongs to the GHKL superfamily (for a review see Dutta, R & Inouye, M. (2000) Trends Biochem. Sci. 25, 24-28), named after the prototypical ATP binding domains of Gyrase B (Wigley, D. B. et al. (1991) Nature 351, 624-629; Brino, L. et al. (2000) J. Biol. Chem. 275, 9468-9475), Hsp90 (Obermann, W. M. J. et al., (1998) J. Cell Biol. 143, 901-910), bacterial histidine kinases (Tanaka, T. et al. (1998) Nature 396, 88-92; Bilwes, A. M. et al. (1999) Cell 96, 131-141), and MutL (Ban, C. et al. (1999) Cell 97, 85-97). Superposition of the homologous domains identifies a 5-stranded mixed β-sheet (corresponding to strands β4, β5, β6, β8 and β7 of PDHK-2) and 4 helices (corresponding to α9, α10, α11 and α12 of PDHK-2) as the conserved structural motif. While PDHK and histidine kinases have identical topologies, the first strand of the central β-sheet and the preceding helix (β4 and α9 in PDHK-2) are transposed to the C-terminus of GyrB, Hsp90, and MutL. The latter also contain an insertion of two antiparallel β-strands between the fifth and the fourth strand of the conserved β-sheet.

The 40 C-terminal residues of PDHK-2, including helix α13, are an extension of the globular ATP binding domain that interact with the regulatory domain and a symmetry related molecule (see below). The interactions between the N-terminal and ATP binding domains of PDHK-2 can be summarised as follows: Loop α1-α2 interacts with loop β8-α13. Helix α2 interacts with strands β1 and β2. The C-terminal portion of helix α3 interacts with loop β8-α3 and helix α13. Helix α7 interacts with loop α11-α2. Helix α8 interacts with loop α11-α12, helix α12 and helix α13.

Between the regulatory and catalytic domain is a large, straight groove, with approximate dimensions of 25 Å in length and 10 Å in diameter (see FIG. 2). The proportion of the groove as well as its vicinity to the ATP binding site suggests that it could be the binding site for a helix of the substrate protein. However, based on the x-ray structure from rat and a model of the lipoyl domain (L2) from rat, it has been proposed that the groove between the regulatory and catalytic domain is the binding site for the lipoyl domain (L2) of the PDH complex (Steussy et al (2001), J. Biol Chem. 276 (40), 37433-37450. Dimer assembly: In the crystal structure, two molecules of PDHK-2 are related by a crystallographic two-fold rotation axis (see FIG. 3, showing monomer A in white, monomer B in black, viewed with the crystallographic two-fold rotation axis vertical in the plane of the paper (top) and along the two-fold rotation axis (bottom). The termini, as far as visible in the electron density maps, of both monomers are labelled.) The corresponding interface buries 2040 Å² of surface area on each molecule, suggesting that it is a biologically relevant molecular dimer interface (Janin, J. (1997) Nat. Struct. Biol. 4, 973-974). Independent evidence for the possible dimeric assembly of PDHK is provided by the finding that the protein, when purified from bovine kidney or rat heart, is a heterodimer of isozymes 1 and 2 (Stepp, L. R. et al. (1983) J. Biol. Chem. 258, 9454-9458; Popov, K. M. et al. (1991) Protein Expression Purif. 2, 278-286; Popov, K. M. et al. (1994) J. Biol. Chem. 269, 29720-29724; Liu, S., Baker, J. C. & Roche, T. E. (1995) J. Biol. Chem. 270, 793-800).

The main contact is formed by the central β-sheets of the catalytic domains. In addition, the 40 C-terminal residues (including helix α13) of PDHK form a highly flexible arm (as indicated by the weak electron density for this region), extending from the catalytic domain of one monomer to the symmetry-related molecule. The following residues are involved in the dimer interaction (i.e. are within 4 Å distance of the symmetry related monomer): residues 28 and 30 of loop α1-α2; residues 153, 157, and 160 of helix α8; residues 227 and 229 of strand β3 and loop β3-β4; residue 276 of strand β5; residues 280-282 of loop β5-β6; residues 283, 285 and 287 of strand β6; residues 296-298 of loop β6-α11 and helix α11; residues 343, 347 and 348 of strand β7; residues 349-352 of loop β7-β8; residues 355 and 359 of strand β8; residues 370-373 of loop β8-α13; residues 375, 377, 378 and 380 of helix α13; residues 381, 385 and 388-392 of the flexible C-terminal dimerisation arm.

The crystal structure reported here could be used to design ligands binding at the proposed dimerisation interface. If dimer formation of PDHK is required for effective PDH complex inactivation, it could be possible to modulate PDH complex activity by regulating the amount of PDHK dimerisation.

(b) Description of the ATP Binding Site: Target for Competitive Inhibition

The structure of PDHK-2* was determined in the presence of ATP and synthetic ligands bound at the ATP binding site of the catalytic domain (see FIG. 4, showing ATP (thick) and a Mg²⁺ ion together with coordinating water molecules at the ATP binding site of PDHK-2. The model is superimposed onto the f_{obs}-f_{calc} difference density map at 2.4 Å (contour level: 2.5σ) calculated prior to incorporation of the ATP: Mg²⁺ complex in the model.). The binding pocket is located between the central β-sheet and the adjacent layer of helices. It is formed by helix α10 (Glu251, Leu252, Lys254, Asn255, Ala256, Arg258, Ala259), strand β7 (Met288, Ser289, Asp290), loop β7α11 (Gly292, Gly293, Gly294, Val295), helix α11 (Ile300, Leu303), loop α11α12 (Leu323, Ala324, Gly325, Phe326), helix α12 (Gly327, Tyr328, Gly329, Leu330, Pro331), strand β7 (Leu346, Ser348) and strand β9 (Thr354, Asp355, Ala356). Residues 313 to 327 (located between helices α11 and α12), constitute the major portion of the so-called ATP-lid. In the absence of ATP they are structurally disordered. Upon binding of ATP residues 323 to 327 become ordered, forming backbone interactions with the β- and γ-phosphate groups of ATP.

The mode by which ATP binds to PDHK-2 closely resembles that of ATP or ADP binding to Gyrase B (Wigley, D. B. et al. (1991) Nature 351, 624-629; Brino, L. et al. (2000) J. Biol. Chem. 275, 9468-9475), Hsp90 (Obermann, W. M. J. et al., (1998) J. Cell Biol. 143, 901-910) and MutL (Ban, C. et al. (1999) Cell 97, 85-97). The binding site can be subdivided into two parts: a polar groove in the surface, binding the phosphoribose moiety of the nucleotide, and a spacious hydrophobic pocket with a polar floor, which accommodates the adenine base. Residues Val295, Leu303, Leu346 and Thr354 line one face of the adenine ring and Asn255, Ala256 and Ala259 flank the opposite face. Asp290 is a conserved, key recognition feature for ATP within the GHKL superfamily. Its carboxylate group acts as hydrogen bond acceptor for the 6-amino group of the adenine ring. Further water-mediated hydrogen bonds complete the interactions between the adenine ring and the protein (FIG. 4).

The ribose and the triphosphate bind to a groove in the surface of the catalytic domain and form direct and indirect interactions with the protein. The 4′-oxygen atom of the ribose ring is in Van der Waals contact with Leu303, whilst the 2′- and 3′-hydroxyl groups form direct or water mediated hydrogen bonds to the side chain of Arg258. The 3′-hydroxyl group stabilises the conformation of the nucleotide by forming an intramolecular hydrogen bond to the β-phosphate group. Binding of ATP is accompanied by binding of a Mg²⁺ ion. Each phosphate group contributes one oxygen atom to the octahedral co-ordination of the Mg²⁺ ion, which is completed by the Oδ1 atom of Asn255 and two water molecules (FIG. 4). Due to the proximity to the N-terminus of helix α12 the negative charges of the tri-phosphate are further compensated by the positive dipole moment of this helix.

It is worth noting that ATP soaked into crystals of PDHK-2* is not hydrolysed. In contrast, GyrB, Hsp90 and MutL show at least moderate ATPase activity (Wang, J. C. (1996) Annu. Rev. Biochem. 65, 635-692; Obermann, W. M. J. et al., (1998) J. Cell Biol. 143, 901-910; Ban, C. & Yang, W. (1998) Cell 95, 541-552). The differences in their catalytical properties and topology allow the distinction between ATPases (GyrB, Hsp90 and MutL, also classified as GHL subgroup) and kinases (histidine kinases and PDHK) within the GHKL superfamily (Ban, C. et al. (1999) Cell 97, 85-97; Dutta, R & Inouye, M. (2000) Trends Biochem. Sci. 25, 24-28).

(c) Description of the Putative E₂L₂ Binding Site

We have determined the structure of PDHK-2* in complex with synthetic PDHK inhibitors, Compound 1 (4-{(2,5)-dimethyl-4-[3,3,3-trifluoro-2-hydroxy-2-methylpropanoyl]piperazinyl}carbonyl)benzonitrile; Aicher. T. et al. (1999) J. Med. Chem. 42, 2741-2746) and Compound 2 (N-{4-[(ethylanilino)sulfonyl]-2-methylphenyl}-3,3,3-trifluoro-2-hydroxy-2-methylpropanamide; Example 12 in patent application WO 99/62873), and identified their binding site (see FIGS. 1, 2 and 5). It resides on the regulatory domain of PDHK and is formed by residues Leu31, Gln35, Phe36, Asp38, and Phe39 of helix α2 and the preceding loop α1α2, residues Thr48, Ser49, Phc52 and Leu53 of helix α3 as well as residues Met167, Leu168, Gln171, His172, Ile175, and Phe176 of helix α8. Loop α1α2, helix α2 and helix a3 form a triangle, the bottom of which is lined by the C-terminal portion of helix α8.

Although both compounds prevent ATP dependent inhibition of the PDH complex, as does exogenous E₂L₂ (Jackson, J. C. (1998) Biochem. J. 334, 703-711), they do not inhibit the kinase activity of PDHK when using a PDHK specific substrate peptide (data not shown). The structure of PDHK-2 shows that the binding pocket of Compounds 1 and 2 is predominantly hydrophobic, which has also been proposed for the site of the lipoyl domain of the E₂ component (E₂L₂) of the PDH complex (Jackson, J. C. (1998) Biochem. J. 334, 703-711). We therefore believe, that the binding site of Compounds 1 and 2 could be identical with, or part of the E₂L₂ binding site of PDHK. Accordingly, this class of compounds could inhibit ATP dependent phosphorylation of the PDH complex by blocking the association between PDHK and the E₂L₂ component of the PDH complex.

The comparison of PDHK native and complex crystal structures reveals conformational differences. In the presence of ligands helix α2 is shifted by a hinge motion around the Cα position of Met33. This opens the binding pocket and allows accommodation of ligands at the site. The conformational differences are most pronounced for residue Phe39. In the absence of ligands the benzene ring of Phe39 occupies the space that otherwise is filled by the central portion of the ligand. Ligand binding induces a 2-2.5 Å shift of its Cα position and flipping of the side chain. The loop α2-α3 (residues 42-45) is found to be very flexible in all structures determined to date. This may be necessary to facilitate the hinge movement of helix α2.

In both crystal structures the (R)-3,3,3-trifluoro-2-hydroxy-2-methylpropionyl moiety of the ligands binds in a similar manner (see FIGS. 5a and 5b). A key recognition feature is a hydrogen bond between the side chain hydroxyl of Ser49 and the 2-hydroxyl group of the ligand. The 2-methyl group extends into a small hydrophobic sub-pocket enclosed by residues Phe39, Ser49, Gln171, His172, Ile175 and Phe176. The trifluoromethyl group forms van der Waals interactions with the side chains of residues Phe52, Leu53, Phe36, Leu168 and Gln171. In the structure with Compound 1, but not with Compound 2, the side chain of Leu31 is also in contact with the trifluoromethyl group, due to steric clashes with its 2,6-dimethylpiperazine group. The amide oxygen of Compound 2 forms a hydrogen bond to a buried water molecule, which bridges the backbone carbonyl of Phe36 and atom Nε2 of Gln171. However, this water molecule could not be detected in the presence of Compound 1 and other inhibitors of this class (data not shown), indicating that it is not essential for ligand binding. The central portion of the ligands is located in a short hydrophobic channel, consisting of residues Leu31, Gln35, Phe39, Ser49 and Phe52, that connects the (R)-3,3,3-trifluoro-2-hydroxy-2-methylpropionyl binding pocket with the protein surface. The 2,6-dimethylpiperazine moiety of Compound 1 and correspondingly the methylbenzyl group of Compound 2 form hydrophobic interactions with the protein. The 4-cyanophenylketone of Compound 1 and the N-benzyl-N-ethylsulfonamide of Compound 2 point towards solvent and are partially disordered in the crystal structures, indicating their flexibility.

Analysis of the aligned sequences of the four PDHK isozymes reveals a residue difference between PDHK isozymes 1-3 with respect to isozyme 4 (Rowles, J. et al (1996) J. Biol. Chem. 271, 22376-82; GenBank accession U54617) at the putative lipoyl binding site. PDHK-2 displays a phenylalanine in position 36, as do PDHK-1 and PDHK-3 at the equivalent positions, but PDHK-4 has a leucine (Leu40) in the equivalent position. This residue exchange has been shown to contribute to the observed potency difference of an analogue of Compound 1 against PDHK-2 and PDHK-4 (data not shown). Furthermore, a single nucleotide polymorphism (SNP) has been identified in some individuals resulting in a Phe43Leu exchange in PDHK-4. The data we present here show that the equivalent residue in PDHK-2 (Phe39) is directly involved in binding of compounds 1 and 2. We therefore conclude, that this SNP could significantly influence the binding affinity of natural or synthetic ligands to the putative E₂L₂ binding site of effected PDHK-4 variants. This analysis highlights the potential of using the described structure of PDHK-2 to identify residues that are critical for ligand binding, and to use this information to genetically engineer site-directed mutants of PDHK-2, designed to mimic any other PDHK isozyme.

(d) Description of a Novel Ligand Binding Site

Soaking of PDHK crystals with another ligand (Compound 3) has led to the identification of a novel, additional ligand binding site (also termed third binding site) on the regulatory domain of PDHK-2 (see FIG. 6 which shows a view of Compound 3 bound to the enzyme, and the structure of Compound 3). The site, which is pre-formed in the native protein, is constituted by Leu71, Pro72, Arg74, Val75 of loop α3-α4 and helix α4, Thr78, Ser80, Val81 of loop α4-α5 and helix α5, Met130, Glyl33, Val134, Glu136, Tyr137 of helix α7, Asp144, Val146, Ser147, Asn150, Ile151, Phe154, Leu155 of loop α7-α8 helix α8 as well as Tyr382 at the C-terminus of helix α13.

(e) Identification and Description of a DCA Binding Site

A binding site of DCA on PDHK-2 has been identified by calculating a difference fourier map with [f_{obs} (ADP/DCA)-f_{obs} (native)] expiα_{calc} (native) as coefficients, where f_{obs} (ADP/DCA) are the observed structure factor amplitudes of PDHK-2* soaked with both ADP and DCA, f_{obs} (native) are the observed structure factor amplitudes of unliganded PDHK-2*, and α_{calc} (native) are the phases derived from the refined model of unliganded PDHK-2* (Example 6).

The resulting f_{obs}(ADP, DCA)-f_{obs} (native) electron density map contains a peak greater than 5.5σ in a small pocket of the regulatory domain of PDHK-2 (FIG. 7). FIG. 7 shows a refined model of DCA bound to the newly identified binding site of PDHK-2. The model is superimposed onto the 2.5 Å f_{obs}(ADP, DCA)-f_{obs}(native) difference density map contoured at 4σ (light grey) and 5σ (dark grey). A hydrogen bond between DCA and Arg162 is indicated. The pocket is formed by residues Leu57, Leu61, Tyr88, Ser91, Ile95, Ile119, Arg120, His123, Ser161, Arg162, Ile165, Arg166, and Ile169. The carboxylate group of DCA forms a salt-bridge with Arg162, while the two chlorine substituents are deeply buried in the hydrophobic portion of the pocket. The binding pocket is close to the surface of the groove between the catalytic and the regulatory domain of PDHK-2 (see FIG. 2), in the vicinity of a cluster of conserved arginine residues (Arg120, Arg162, Arg166). The f_{obs}(ADP, DCA)-f_{obs}(native) map does contain a further peak greater than 4σ at the position of the β-phosphate of ADP as well as less dominant peaks for other parts of the bound ADP molecule. A peak of more than 4 σ is also observed for the side chain of Glu251, which is involved in the coordination of the ADP associated Mg²⁺ ion and is flipped with respect to its conformation in the absence of ADP/Mg²⁺. Since the f_{obs}(ADP, DCA)-f_{obs}(native) electron density map does not contain further significant peaks, we conclude that the described DCA binding pocket is the only, or at least the major binding site of DCA on PDHK-2.

The shape (see Example 8) and charge distribution of the groove as well as the high occurence of conserved residues on its surface suggest that it is of functional importance, e.g. for binding of PDHK to a subunit or domain of the PDH complex. We therefore hypothesize that DCA could inhibit the phosphorylation of the PDH complex, by interfering with the binding of one of its components to PDHK.

The DCA binding pocket of PDHK-2 identified herein could be utilised for the design of synthetic ligands that inhibit phosphorylation of the PDH complex.

### Example 9

### Assay to Measure Whether Compounds Inhibit PDHK Activity

Compounds were assessed for inhibition of PDHK in an assay adapted from Espinal, J. et al (1995) Drug Development Res. 35, 130-136. In the first part of the assay, compounds were incubated with porcine PDH (Sigma P-7032) and MgATP, and the reaction was stopped after defined intervals. In the second part of the assay, the residual PDH activity was measured by monitoring the rate of reduction of NAD at 340 nm.

The assay was performed in flat-bottom 96-well microtitre plates, using a final volume of 50 μl for the first part of the assay (kinase reaction), and 250 μl for the PDH reaction. At first, 10 μl test compound in a suitable diluent, 20 μl kinase buffer (20 mM potassium phosphate, pH7.0, 4 mM MgCl₂, 4 mM dithiothreitol) and 10 μl PDH (Sigma, P-7032, diluted with water to a final concentration of 1.83 mg/ml) were mixed and incubated for 10 min; then 10 μl ATP solution (125 μM in kinase buffer) was added to start the reaction.

The incubations were done at 30° C. The kinase reaction was terminated after defined intervals (usually 0, 10, 20, 30, 60, and 90 min) by initiating the PDH reaction. This was done by addition of 200 μl of PDH substrate solution (1 mM thiamine pyrophosphate, 0.5 mM NAD, 0.5 mM coenzyme A, 12 mM pyruvate, 5 mM dithiothreitol). Kinetic readings were then taken at 340 nm conducted at 12 sec intervals for 20 min with shaking for 1 sec before each reading was taken, in a SpectraMax plate reader. The data were processed using the Spectramax software.

### Example 10

### Synthesis of Compound 3

(a) Methyl {3-chloro-4-[(4-isopropylbenzyl)oxy]phenyl}acetate

A stirred solution of 4-isopropylbenzyl alcohol (4.5 ml, 29.1 mmoles), 3-chloro-4-hydroxybenzeneacetic acid, methyl ester (CAS registry number 57017-95-5) and triphenylphosphine (8.8 g, 33.6 mmoles) in dry tetrahydrofuran (100 ml) was cooled to 0° C. Diisopropyl azodicarboxylate (5.7 ml, 29.1 mmoles) was added dropwise over 10 minutes and the reaction was stirred for a further 3 hours at room temperature. The solvent was removed under reduced pressure and the product purified on silica, eluting with pentane: ethyl acetate (4:1) to yield the title compound as a colourless oil (2.2 g, 30%).

¹H NMR (400 MHz, CDCl₃) δ1.25(6H, d), 2.90(1H, m), 3.55(2H, s), 3.70(3H, s), 5.10(2H, s), 6.95(1H, d), 7.10(1H, d), 7.22(2H, d), 7.30(1H, s), 7.38(2H, d). ms 350, 352 (MNH₄⁺ for both Cl isotopes). Found: C, 67.95; H, 6.47. C₁₉H₂₁ClO₃.0.2 ethyl acetate requires C, 67.86; H, 6.50.

(b) 3-Chloro-4-[(4-isopropylbenzyl)oxy]phenyl}acetic acid

A mixture of methyl {3-chloro-4-[(4-isopropylbenzyl)oxy]phenyl}acetate (2.2 g, 6.6 mmoles) in aqueous sodium hydroxide (1 M, 20 ml) and dioxan (20 ml) was stirred at room temperature for 1 hour. The dioxan was then removed under reduced pressure, the remaining aqueous solution acidified with aqueous hydrochloric acid (1M, 25 ml) and the resultant white solid isolated by filtration. The solid was then azeotroped with ethanol to afford the title compound as a white solid (1.9 g, 90%).

¹H NMR (300 MHz, CDCl₃) δ2.15(6H, d), 2.95(1H, m), 3.57(2H, s), 5.10(2H, s), 6.95(1H, d), 7.10(1H, d), 7.22(2H, d), 7.30(1H, s), 7.38(2H, d). ms 336 (MNH₄⁺) Found: C, 66.87; H, 6.23. C₁₈H₁₉ClO₃.0.4 ethanol requires C, 66.96; H, 6.40.

(c) tert-butyl 2-[({3-chloro-4-[(4-isopropylbenzyl)oxy]phenyl}acetyl)amino]ethylcarbamate

3-Chloro-4-[(4-isopropylbenzyl)oxy]phenyl}acetic acid (0.240 g, 0.75 mmoles), N-methylmorpholine (0.240 ml, 2.5 mmoles), O-(7-azabenzotriazol-1-yl)-N,N,N′,N′-tetramethyluronium hexafluorophosphate (0.32 g, 0.85 mmoles) and N-BOC-ethylenediamine (0.12 ml, 0.75 mmole) were stirred in acetonitrile at room temperature for 72 hours. The solvent was removed under reduced pressure. Purification using flash chromatography on silica eluting with pentane: ethyl acetate (1:2) yielded the title compound (0.1 85 g, 54%), which was used without further purification.

¹H NMR (400 MHz, CDCl₃) δ1.21(6H, d), 1.37(9H,s), 2.87(1H, m), 3.18(2H, m), 3.27(2H, m), 3.41(2H, s), 4.80(1H, s), 5.04(2H, s), 6.08(1H, s), 6.90(1H, d), 7.04(1H, m), 7.24(3H, m), 7.35(2H, d). ms 483 (MNa⁺)

(d) N-(2-aminoethyl)-2-{3-chloro-4-[(4-isopropylbenzyl)oxy]phenyl}acetamide

tert-Butyl 2-[({3-chloro-4-[(4-isopropylbenzyl)oxy]phenyl}acetyl)amino]ethylcarbamate (0.185 g, 0.41 mmoles) was dissolved in dichloromethane. The solution was saturated with gaseous hydrogen chloride and the reaction stirred at room temperature for 1 hour. The solvent was removed under reduced pressure. The residue was dissolved in dichloromethane, washed with saturated sodium hydrogen carbonate, dried (magnesium sulphate) and the solvent removed under reduced pressure. Purification using flash chromatography on silica eluting with dichloromethane: methanol: 0.880ammonia (90:10:1) yielded the title compound (0.035 g, 24%) as a white solid.

¹H NMR (400 MHz, CDCl₃) δ1.24(6H, d), 2.78(2H, t), 2.92(1H, m), 3.24(2H, m), 3.46(2H, s), 5.10(2H, s), 5.88(1H, s), 6.94(1H, d), 7.07(1H, m), 7.23(2H, m), 7.30 (1H, s), 7.37(2H, d). ms ES-359 (M−1) Found: C, 63.46; H, 7.10; N, 7.30: C₂₀H₂₅ClN₂O₂.0.24 CH₂Cl₂ requires C, 63.76; H, 6.74; N, 7.35.

TABLE 1
Data collection and phasing statistics

| | | 1 mM | 5 mM | 10 mM | Compound 1 | | | | |
|---|---|---|---|---|---|---|---|---|---|
| Resolution [Å] | 2.2 | 2.8 | 3.0 | 2.8 | 2.4 | 2.35 | 2.4 | 2.4 | 2.5 |
| Unique reflections | 28844 | 14054 | 11314 | 14047 | 21971 | 23185 | 21614 | 21046 | 20370 |
| Completeness [%] | 97.9 | 99.0 | 100.0 | 99.7 | 98.5 | 97.6 | 97.1 | 94.3 | 99.2 |
| Redundancy | 3.7 | 7.0 | 8.3 | 8.7 | 3.4 | 2.9 | 3.8 | 7.3 | 3.5 |
| Refl. < 3σ [%] | 90.9 | 88.8 | 90.7 | 90.1 | 79.7 | 83.3 | 87.0 | 87.7 | 84.7 |
| R_{sym}^{a} | 0.035 | 0.079 | 0.060 | 0.062 | 0.054 | 0.048 | 0.061 | 0.060 | 0.043 |
| Rᵢₛₒ^{b} | | 0.234 | 0.285 | 0.237 | | | | | |
| R_{cullis}^{c} | | 0.760 | 0.825 | 0.891 | | | | | |
| Number of sites | | 5 | 3 | 5 | | | | | |
| Phasing power^{d} | | | | | | | | | |
| isomorphous^{e} | | 1.51/1.21 | 1.43/1.37 | 1.23/1.06 | | | | | |
| anomalous^{f} | | 1.49 | 1.64 | 1.36 | | | | | |

${}^{a} {R}_{sym} = {\sum }_{hkl} {\sum }_{i} \vert {I}_{i} ( hkl ) - \overline{I ( hkl )} \vert / {\sum }_{hkl} {\sum }_{i} \vert {I}_{i} ( hkl ) \vert$
${}^{b} {R}_{iso} = {\sum }_{hkl} \vert \vert {F}_{PH} ( hkl ) \vert - \vert {F}_{P} ( hkl ) \vert \vert / \sum \vert {F}_{P} ( hkl ) \vert$
^{c}R_{cullis} = <phase integrated lack of closure >/<|F_{PH}-F_{P}|>.
For centric reflections only.
^{d}Phasing power = <{|FH(calc)|/phase-integrated lack of closure}>
^{e}Pairs of values are given for acentric and centric reflections, respectively.
^{f}For acentric reflections only

TABLE 2
Data refinement statistics

| | | ATP/ | | | | |
|---|---|---|---|---|---|---|
| | Native | Compound 1 | Compound 1 | Compound 2 | Compound 3 | ADP/DCA |
| Resolution range, Å | 30.0-2.2 | 30.0-2.4 | 30.0-2.35 | 30.0-2.4 | 30.0-2.4 | 30.0-2.5 |
| Amino acid residues, | 356 | 357 | 351 | 352 | 357 | 358 |
| no. | | | | | | |
| Water molecules, no. | none | 91 | 111 | 60 | 153 | 151 |
| Ligand molecules | none | ATP, Mg²⁺, | Compound 1 | Compound 2 | Compound 3 | ADP, Mg²⁺, DCA |
| | | Compound 1 | | | | |
| rmsd bond length, Å | 0.008 | 0.009 | 0.008 | 0.008 | 0.009 | 0.010 |
| rmsd bond anlges, ° | 1.290 | 1.635 | 1.342 | 1.309 | 1.356 | 1.295 |
| R-factor | 0.267 | 0.238 | 0.237 | 0.247 | 0.211 | 0.248 |
| Free R-factor (5% of | 0.297 | 0.247 | 0.255 | 0.261 | 0.241 | 0.262 |
| data) | | | | | | |

$R = {\sum }_{hkl} \vert {F}_{obs} \vert - k \vert {F}_{calc} ‖ / {\sum }_{hkl} \vert {F}_{obs} \vert$

TABLE 3
Atomic coordinates for apo PDHK2* (numbering as in FIG. 1)

REMARK coordinates from restrained individual B-factor refinement
REMARK refinement resolution: 30 - 2.2 A

| | | |
|---|---|---|
| REMARK starting | r = 0.2655 | free r = 0.2962 |
| REMARK final | r = 0.2658 | free r = 0.2965 |

| | | |
|---|---|---|
| REMARK B rmsd for bonded mainchain atoms = | 2.488 | target = 1.5 |
| REMARK B rmsd for bonded sidechain atoms = | 3.467 | target = 2.0 |
| REMARK B rmsd for angle mainchain atoms = | 4.028 | target = 2.0 |
| REMARK B rmsd for angle sidechain atoms = | 5.234 | target = 2.5 |

REMARK wa = 1.90983
REMARK rweight = 4.84665E−02
REMARK target = mlhl steps = 15
REMARK sg = P6(4) a = 109.317 b = 109.317 c = 85.087 alpha = 90 beta = 90 gamma = 120
REMARK parameter file 1: MSI_CNX_TOPPAR: protein rep.param
REMARK molecular structure file: ../generate/generate.psf
REMARK input coordinates: minimize.pdb
REMARK reflection file = ../pdhk18_dar.cv
REMARK ncs = none
REMARK B-correction resolution: 6.0 - 2.2
REMARK initial B-factor correction applied to fobs:

| | | | | | | |
|---|---|---|---|---|---|---|
| REMARK | B11 = | −3.137 | B22 = | −3.137 | B33 = | 6.273 |
| REMARK | B12 = | −6.098 | B13 = | 0.000 | B23 = | 0.000 |

REMARK B-factor correction applied to coordinate array B: −0.156
REMARK bulk solvent: (Mask) density level = 0.34731 e/A\hat{ }3, B-factor = 44.3891 A\hat{ }2
REMARK reflections with |Fobs|/sigma_F < 0.0 rejected
REMARK reflections with |Fobs| > 10000 * rms(Fobs) rejected

| | | |
|---|---|---|
| REMARK theoretical total number of refl. in resol. range: | 29414 | (100.0%) |
| REMARK number of unobserved reflections (no entry or \|F\| = 0): | 183 | (0.6%) |
| REMARK number of reflections rejected: | 0 | (0.0%) |
| REMARK total number of reflections used: | 29231 | (99.4%) |
| REMARK number of reflections in working set: | 27802 | (94.5%) |
| REMARK number of reflections in test set: | 1429 | (4.9%) |
| REMARK FILENAME = “bindividual.pdb” | | |
| REMARK DATE: 3 APR. 2001 17:24:40 created by user: knoechel | | |
| REMARK Written by CNX VERSION: 2000 | | |

| | | | | | | | | | | | | |
|---|---|---|---|---|---|---|---|---|---|---|---|---|
| ATOM | 1 | C | GLY | A | 14 | 61.054 | 42.675 | 101.842 | 1.00 | 54.17 | A | C |
| ATOM | 2 | O | GLY | A | 14 | 60.321 | 42.912 | 102.805 | 1.00 | 54.94 | A | O |
| ATOM | 3 | N | GLY | A | 14 | 63.155 | 41.575 | 101.084 | 1.00 | 52.43 | A | N |
| ATOM | 4 | CA | GLY | A | 14 | 61.857 | 41.389 | 101.785 | 1.00 | 51.86 | A | C |
| ATOM | 5 | N | SER | A | 15 | 61.178 | 43.509 | 100.813 | 1.00 | 55.73 | A | N |
| ATOM | 6 | CA | SER | A | 15 | 60.450 | 44.778 | 100.793 | 1.00 | 60.92 | A | C |
| ATOM | 7 | CB | SER | A | 15 | 61.446 | 45.942 | 100.902 | 1.00 | 64.21 | A | C |
| ATOM | 8 | OG | SER | A | 15 | 62.521 | 45.787 | 99.984 | 1.00 | 70.83 | A | O |
| ATOM | 9 | C | SER | A | 15 | 59.516 | 45.004 | 99.594 | 1.00 | 62.26 | A | C |
| ATOM | 10 | O | SER | A | 15 | 59.115 | 46.142 | 99.324 | 1.00 | 64.31 | A | O |
| ATOM | 11 | N | ALA | A | 16 | 59.154 | 43.935 | 98.886 | 1.00 | 59.76 | A | N |
| ATOM | 12 | CA | ALA | A | 16 | 58.269 | 44.073 | 97.732 | 1.00 | 58.24 | A | C |
| ATOM | 13 | CB | ALA | A | 16 | 58.094 | 42.729 | 97.022 | 1.00 | 58.21 | A | C |
| ATOM | 14 | C | ALA | A | 16 | 56.911 | 44.632 | 98.154 | 1.00 | 56.97 | A | C |
| ATOM | 15 | O | ALA | A | 16 | 56.445 | 45.619 | 97.588 | 1.00 | 55.41 | A | O |
| ATOM | 16 | N | PRO | A | 17 | 56.262 | 44.014 | 99.158 | 1.00 | 56.95 | A | N |
| ATOM | 17 | CD | PRO | A | 17 | 56.659 | 42.788 | 99.875 | 1.00 | 57.06 | A | C |
| ATOM | 18 | CA | PRO | A | 17 | 54.954 | 44.486 | 99.629 | 1.00 | 58.43 | A | C |
| ATOM | 19 | CB | PRO | A | 17 | 54.722 | 43.656 | 100.888 | 1.00 | 56.32 | A | C |
| ATOM | 20 | CG | PRO | A | 17 | 55.362 | 42.357 | 100.535 | 1.00 | 57.00 | A | C |
| ATOM | 21 | C | PRO | A | 17 | 54.946 | 45.978 | 99.928 | 1.00 | 60.78 | A | C |
| ATOM | 22 | O | PRO | A | 17 | 53.978 | 46.684 | 99.627 | 1.00 | 60.86 | A | O |
| ATOM | 23 | N | LYS | A | 18 | 56.036 | 46.446 | 100.526 | 1.00 | 63.17 | A | N |
| ATOM | 24 | CA | LYS | A | 18 | 56.182 | 47.849 | 100.889 | 1.00 | 64.85 | A | C |
| ATOM | 25 | CB | LYS | A | 18 | 57.525 | 48.062 | 101.596 | 1.00 | 68.13 | A | C |
| ATOM | 26 | CG | LYS | A | 18 | 57.751 | 49.467 | 102.146 | 1.00 | 70.28 | A | C |
| ATOM | 27 | CD | LYS | A | 18 | 59.158 | 49.592 | 102.735 | 1.00 | 72.96 | A | C |
| ATOM | 28 | CE | LYS | A | 18 | 59.440 | 51.003 | 103.234 | 1.00 | 73.49 | A | C |
| ATOM | 29 | NZ | LYS | A | 18 | 60.856 | 51.161 | 103.662 | 1.00 | 72.33 | A | N |
| ATOM | 30 | C | LYS | A | 18 | 56.087 | 48.759 | 99.667 | 1.00 | 63.10 | A | C |
| ATOM | 31 | O | LYS | A | 18 | 55.333 | 49.730 | 99.670 | 1.00 | 60.38 | A | O |
| ATOM | 32 | N | TYR | A | 19 | 56.859 | 48.444 | 98.631 | 1.00 | 63.55 | A | N |
| ATOM | 33 | CA | TYR | A | 19 | 56.852 | 49.241 | 97.405 | 1.00 | 67.43 | A | C |
| ATOM | 34 | CB | TYR | A | 19 | 57.814 | 48.656 | 96.375 | 1.00 | 72.59 | A | C |
| ATOM | 35 | CG | TYR | A | 19 | 59.248 | 48.626 | 96.822 | 1.00 | 78.26 | A | C |
| ATOM | 36 | CD1 | TYR | A | 19 | 59.832 | 49.741 | 97.415 | 1.00 | 80.63 | A | C |
| ATOM | 37 | CE1 | TYR | A | 19 | 61.165 | 49.735 | 97.798 | 1.00 | 84.48 | A | C |
| ATOM | 38 | CD2 | TYR | A | 19 | 60.033 | 47.494 | 96.625 | 1.00 | 80.64 | A | C |
| ATOM | 39 | CE2 | TYR | A | 19 | 61.370 | 47.474 | 97.003 | 1.00 | 85.24 | A | C |
| ATOM | 40 | CZ | TYR | A | 19 | 61.930 | 48.598 | 97.589 | 1.00 | 85.97 | A | C |
| ATOM | 41 | OH | TYR | A | 19 | 63.255 | 48.594 | 97.960 | 1.00 | 90.18 | A | O |
| ATOM | 42 | C | TYR | A | 19 | 55.466 | 49.298 | 96.788 | 1.00 | 65.88 | A | C |
| ATOM | 43 | O | TYR | A | 19 | 54.927 | 50.373 | 96.524 | 1.00 | 65.95 | A | O |
| ATOM | 44 | N | ILE | A | 20 | 54.904 | 48.122 | 96.545 | 1.00 | 64.22 | A | N |
| ATOM | 45 | CA | ILE | A | 20 | 53.581 | 48.022 | 95.962 | 1.00 | 63.95 | A | C |
| ATOM | 46 | CB | ILE | A | 20 | 53.062 | 46.567 | 96.025 | 1.00 | 61.91 | A | C |
| ATOM | 47 | CG2 | ILE | A | 20 | 51.644 | 46.497 | 95.482 | 1.00 | 61.38 | A | C |
| ATOM | 48 | CG1 | ILE | A | 20 | 54.003 | 45.652 | 95.232 | 1.00 | 60.34 | A | C |
| ATOM | 49 | CD1 | ILE | A | 20 | 53.642 | 44.189 | 95.292 | 1.00 | 58.50 | A | C |
| ATOM | 50 | C | ILE | A | 20 | 52.632 | 48.940 | 96.726 | 1.00 | 64.88 | A | C |
| ATOM | 51 | O | ILE | A | 20 | 51.843 | 49.672 | 96.125 | 1.00 | 62.13 | A | O |
| ATOM | 52 | N | GLU | A | 21 | 52.729 | 48.909 | 98.052 | 1.00 | 65.57 | A | N |
| ATOM | 53 | CA | GLU | A | 21 | 51.877 | 49.736 | 98.895 | 1.00 | 67.52 | A | C |
| ATOM | 54 | CB | GLU | A | 21 | 52.075 | 49.381 | 100.368 | 1.00 | 71.49 | A | C |
| ATOM | 55 | CG | GLU | A | 21 | 51.134 | 50.120 | 101.311 | 1.00 | 79.40 | A | C |
| ATOM | 56 | CD | GLU | A | 21 | 49.669 | 49.966 | 100.921 | 1.00 | 84.34 | A | C |
| ATOM | 57 | OE1 | GLU | A | 21 | 49.203 | 48.813 | 100.763 | 1.00 | 85.42 | A | O |
| ATOM | 58 | OE2 | GLU | A | 21 | 48.985 | 51.003 | 100.774 | 1.00 | 86.30 | A | O |
| ATOM | 59 | C | GLU | A | 21 | 52.166 | 51.217 | 98.685 | 1.00 | 66.11 | A | C |
| ATOM | 60 | O | GLU | A | 21 | 51.266 | 52.055 | 98.778 | 1.00 | 67.36 | A | O |
| ATOM | 61 | N | HIS | A | 22 | 53.420 | 51.536 | 98.390 | 1.00 | 61.53 | A | N |
| ATOM | 62 | CA | HIS | A | 22 | 53.806 | 52.922 | 98.174 | 1.00 | 58.56 | A | C |
| ATOM | 63 | CB | HIS | A | 22 | 55.323 | 53.071 | 98.261 | 1.00 | 54.18 | A | C |
| ATOM | 64 | CG | HIS | A | 22 | 55.809 | 54.450 | 97.947 | 1.00 | 55.62 | A | C |
| ATOM | 65 | CD2 | HIS | A | 22 | 56.713 | 54.894 | 97.041 | 1.00 | 56.76 | A | C |
| ATOM | 66 | ND1 | HIS | A | 22 | 55.345 | 55.569 | 98.608 | 1.00 | 55.64 | A | N |
| ATOM | 67 | CE1 | HIS | A | 22 | 55.943 | 56.643 | 98.120 | 1.00 | 54.92 | A | C |
| ATOM | 68 | NE2 | HIS | A | 22 | 56.778 | 56.262 | 97.169 | 1.00 | 56.91 | A | N |
| ATOM | 69 | C | HIS | A | 22 | 53.335 | 53.474 | 96.831 | 1.00 | 57.23 | A | C |
| ATOM | 70 | O | HIS | A | 22 | 52.564 | 54.430 | 96.778 | 1.00 | 58.61 | A | O |
| ATOM | 71 | N | PHE | A | 23 | 53.806 | 52.874 | 95.746 | 1.00 | 55.11 | A | N |
| ATOM | 72 | CA | PHE | A | 23 | 53.444 | 53.346 | 94.418 | 1.00 | 55.06 | A | C |
| ATOM | 73 | CB | PHE | A | 23 | 54.353 | 52.710 | 93.374 | 1.00 | 55.08 | A | C |
| ATOM | 74 | CG | PHE | A | 23 | 55.805 | 53.053 | 93.552 | 1.00 | 57.82 | A | C |
| ATOM | 75 | CD1 | PHE | A | 23 | 56.643 | 52.228 | 94.297 | 1.00 | 60.34 | A | C |
| ATOM | 76 | CD2 | PHE | A | 23 | 56.336 | 54.205 | 92.976 | 1.00 | 56.60 | A | C |
| ATOM | 77 | CE1 | PHE | A | 23 | 57.995 | 52.543 | 94.463 | 1.00 | 61.00 | A | C |
| ATOM | 78 | CE2 | PHE | A | 23 | 57.681 | 54.532 | 93.135 | 1.00 | 57.24 | A | C |
| ATOM | 79 | CZ | PHE | A | 23 | 58.514 | 53.700 | 93.878 | 1.00 | 61.29 | A | C |
| ATOM | 80 | C | PHE | A | 23 | 51.983 | 53.145 | 94.031 | 1.00 | 52.09 | A | C |
| ATOM | 81 | O | PHE | A | 23 | 51.494 | 53.814 | 93.129 | 1.00 | 52.07 | A | O |
| ATOM | 82 | N | SER | A | 24 | 51.282 | 52.244 | 94.710 | 1.00 | 50.43 | A | N |
| ATOM | 83 | CA | SER | A | 24 | 49.877 | 52.023 | 94.394 | 1.00 | 54.72 | A | C |
| ATOM | 84 | CB | SER | A | 24 | 49.369 | 50.729 | 95.035 | 1.00 | 55.34 | A | C |
| ATOM | 85 | OG | SER | A | 24 | 48.890 | 50.957 | 96.348 | 1.00 | 56.28 | A | O |
| ATOM | 86 | C | SER | A | 24 | 49.039 | 53.201 | 94.894 | 1.00 | 57.02 | A | C |
| ATOM | 87 | O | SER | A | 24 | 47.847 | 53.293 | 94.614 | 1.00 | 56.21 | A | O |
| ATOM | 88 | N | LYS | A | 25 | 49.665 | 54.099 | 95.647 | 1.00 | 60.38 | A | N |
| ATOM | 89 | CA | LYS | A | 25 | 48.965 | 55.269 | 96.170 | 1.00 | 63.45 | A | C |
| ATOM | 90 | CB | LYS | A | 25 | 49.687 | 55.811 | 97.410 | 1.00 | 66.93 | A | C |
| ATOM | 91 | CG | LYS | A | 25 | 49.669 | 54.852 | 98.593 | 1.00 | 73.50 | A | C |
| ATOM | 92 | CD | LYS | A | 25 | 50.524 | 55.357 | 99.750 | 1.00 | 79.17 | A | C |
| ATOM | 93 | CE | LYS | A | 25 | 50.541 | 54.353 | 100.906 | 1.00 | 83.05 | A | C |
| ATOM | 94 | NZ | LYS | A | 25 | 51.515 | 54.717 | 101.981 | 1.00 | 83.82 | A | N |
| ATOM | 95 | C | LYS | A | 25 | 48.899 | 56.343 | 95.092 | 1.00 | 61.35 | A | C |
| ATOM | 96 | O | LYS | A | 25 | 48.158 | 57.313 | 95.209 | 1.00 | 62.68 | A | O |
| ATOM | 97 | N | PHE | A | 26 | 49.687 | 56.155 | 94.042 | 1.00 | 60.71 | A | N |
| ATOM | 98 | CA | PHE | A | 26 | 49.737 | 57.088 | 92.927 | 1.00 | 61.78 | A | C |
| ATOM | 99 | CB | PHE | A | 26 | 51.166 | 57.197 | 92.385 | 1.00 | 67.20 | A | C |
| ATOM | 100 | CG | PHE | A | 26 | 52.137 | 57.814 | 93.343 | 1.00 | 75.97 | A | C |
| ATOM | 101 | CD1 | PHE | A | 26 | 52.305 | 57.295 | 94.623 | 1.00 | 79.65 | A | C |
| ATOM | 102 | CD2 | PHE | A | 26 | 52.898 | 58.917 | 92.960 | 1.00 | 79.44 | A | C |
| ATOM | 103 | CE1 | PHE | A | 26 | 53.217 | 57.867 | 95.512 | 1.00 | 82.04 | A | C |
| ATOM | 104 | CE2 | PHE | A | 26 | 53.812 | 59.496 | 93.838 | 1.00 | 80.32 | A | C |
| ATOM | 105 | CZ | PHE | A | 26 | 53.971 | 58.970 | 95.117 | 1.00 | 81.77 | A | C |
| ATOM | 106 | C | PHE | A | 26 | 48.835 | 56.609 | 91.793 | 1.00 | 60.17 | A | C |
| ATOM | 107 | O | PHE | A | 26 | 48.433 | 55.440 | 91.737 | 1.00 | 54.61 | A | O |
| ATOM | 108 | N | SER | A | 27 | 48.518 | 57.523 | 90.887 | 1.00 | 58.00 | A | N |
| ATOM | 109 | CA | SER | A | 27 | 47.709 | 57.179 | 89.732 | 1.00 | 53.13 | A | C |
| ATOM | 110 | CB | SER | A | 27 | 46.525 | 58.128 | 89.598 | 1.00 | 54.82 | A | C |
| ATOM | 111 | OG | SER | A | 27 | 45.558 | 57.834 | 90.589 | 1.00 | 61.23 | A | O |
| ATOM | 112 | C | SER | A | 27 | 48.586 | 57.261 | 88.497 | 1.00 | 49.42 | A | C |
| ATOM | 113 | O | SER | A | 27 | 49.402 | 58.178 | 88.358 | 1.00 | 46.46 | A | O |
| ATOM | 114 | N | PRO | A | 28 | 48.474 | 56.267 | 87.603 | 1.00 | 46.53 | A | N |
| ATOM | 115 | CD | PRO | A | 28 | 47.803 | 54.961 | 87.712 | 1.00 | 43.21 | A | C |
| ATOM | 116 | CA | PRO | A | 28 | 49.312 | 56.350 | 86.404 | 1.00 | 45.74 | A | C |
| ATOM | 117 | CB | PRO | A | 28 | 48.974 | 55.061 | 85.635 | 1.00 | 45.23 | A | C |
| ATOM | 118 | CG | PRO | A | 28 | 47.693 | 54.557 | 86.278 | 1.00 | 43.37 | A | C |
| ATOM | 119 | C | PRO | A | 28 | 48.950 | 57.631 | 85.643 | 1.00 | 44.55 | A | C |
| ATOM | 120 | O | PRO | A | 28 | 47.866 | 58.187 | 85.832 | 1.00 | 42.59 | A | O |
| ATOM | 121 | N | SER | A | 29 | 49.863 | 58.103 | 84.809 | 1.00 | 43.27 | A | N |
| ATOM | 122 | CA | SER | A | 29 | 49.645 | 59.319 | 84.048 | 1.00 | 46.61 | A | C |
| ATOM | 123 | CB | SER | A | 29 | 50.819 | 60.271 | 84.254 | 1.00 | 48.26 | A | C |
| ATOM | 124 | OG | SER | A | 29 | 51.024 | 60.505 | 85.634 | 1.00 | 55.88 | A | O |
| ATOM | 125 | C | SER | A | 29 | 49.487 | 59.008 | 82.560 | 1.00 | 48.52 | A | C |
| ATOM | 126 | O | SER | A | 29 | 50.464 | 58.742 | 81.853 | 1.00 | 46.83 | A | O |
| ATOM | 127 | N | PRO | A | 30 | 48.246 | 59.043 | 82.065 | 1.00 | 46.84 | A | N |
| ATOM | 128 | CD | PRO | A | 30 | 47.002 | 59.349 | 82.791 | 1.00 | 47.98 | A | C |
| ATOM | 129 | CA | PRO | A | 30 | 47.985 | 58.760 | 80.652 | 1.00 | 47.47 | A | C |
| ATOM | 130 | CB | PRO | A | 30 | 46.459 | 58.737 | 80.587 | 1.00 | 48.43 | A | C |
| ATOM | 131 | CG | PRO | A | 30 | 46.065 | 59.705 | 81.665 | 1.00 | 49.58 | A | C |
| ATOM | 132 | C | PRO | A | 30 | 48.603 | 59.821 | 79.757 | 1.00 | 47.38 | A | C |
| ATOM | 133 | O | PRO | A | 30 | 48.481 | 61.008 | 80.029 | 1.00 | 48.52 | A | O |
| ATOM | 134 | N | LEU | A | 31 | 49.283 | 59.396 | 78.701 | 1.00 | 47.22 | A | N |
| ATOM | 135 | CA | LEU | A | 31 | 49.904 | 60.343 | 77.790 | 1.00 | 47.98 | A | C |
| ATOM | 136 | CB | LEU | A | 31 | 51.352 | 59.945 | 77.512 | 1.00 | 49.05 | A | C |
| ATOM | 137 | CG | LEU | A | 31 | 52.267 | 59.666 | 78.702 | 1.00 | 48.94 | A | C |
| ATOM | 138 | CD1 | LEU | A | 31 | 53.641 | 59.272 | 78.179 | 1.00 | 48.49 | A | C |
| ATOM | 139 | CD2 | LEU | A | 31 | 52.357 | 60.892 | 79.591 | 1.00 | 49.19 | A | C |
| ATOM | 140 | C | LEU | A | 31 | 49.157 | 60.435 | 76.458 | 1.00 | 52.58 | A | C |
| ATOM | 141 | O | LEU | A | 31 | 48.580 | 59.451 | 75.979 | 1.00 | 51.45 | A | O |
| ATOM | 142 | N | SER | A | 32 | 49.181 | 61.624 | 75.861 | 1.00 | 54.42 | A | N |
| ATOM | 143 | CA | SER | A | 32 | 48.532 | 61.856 | 74.576 | 1.00 | 56.87 | A | C |
| ATOM | 144 | CB | SER | A | 32 | 48.244 | 63.345 | 74.369 | 1.00 | 55.29 | A | C |
| ATOM | 145 | OG | SER | A | 32 | 49.422 | 64.028 | 73.957 | 1.00 | 52.50 | A | O |
| ATOM | 146 | C | SER | A | 32 | 49.503 | 61.399 | 73.498 | 1.00 | 57.77 | A | C |
| ATOM | 147 | O | SER | A | 32 | 50.691 | 61.222 | 73.763 | 1.00 | 57.15 | A | O |
| ATOM | 148 | N | MET | A | 33 | 48.995 | 61.219 | 72.284 | 1.00 | 60.20 | A | N |
| ATOM | 149 | CA | MET | A | 33 | 49.827 | 60.801 | 71.171 | 1.00 | 61.89 | A | C |
| ATOM | 150 | CB | MET | A | 33 | 48.959 | 60.484 | 69.956 | 1.00 | 64.11 | A | C |
| ATOM | 151 | CG | MET | A | 33 | 49.687 | 59.772 | 68.834 | 1.00 | 67.18 | A | C |
| ATOM | 152 | SD | MET | A | 33 | 49.472 | 57.991 | 68.928 | 1.00 | 71.74 | A | S |
| ATOM | 153 | CE | MET | A | 33 | 50.798 | 57.561 | 70.006 | 1.00 | 75.48 | A | C |
| ATOM | 154 | C | MET | A | 33 | 50.754 | 61.965 | 70.849 | 1.00 | 63.42 | A | C |
| ATOM | 155 | O | MET | A | 33 | 51.823 | 61.784 | 70.267 | 1.00 | 64.44 | A | O |
| ATOM | 156 | N | LYS | A | 34 | 50.335 | 63.167 | 71.236 | 1.00 | 65.39 | A | N |
| ATOM | 157 | CA | LYS | A | 34 | 51.128 | 64.364 | 70.986 | 1.00 | 65.68 | A | C |
| ATOM | 158 | CB | LYS | A | 34 | 50.288 | 65.625 | 71.221 | 1.00 | 70.89 | A | C |
| ATOM | 159 | CG | LYS | A | 34 | 50.632 | 66.792 | 70.280 | 1.00 | 76.11 | A | C |
| ATOM | 160 | CD | LYS | A | 34 | 52.051 | 67.333 | 70.485 | 1.00 | 79.07 | A | C |
| ATOM | 161 | CE | LYS | A | 34 | 52.154 | 68.229 | 71.714 | 1.00 | 78.89 | A | C |
| ATOM | 162 | NZ | LYS | A | 34 | 51.331 | 69.466 | 71.568 | 1.00 | 77.37 | A | N |
| ATOM | 163 | C | LYS | A | 34 | 52.326 | 64.354 | 71.922 | 1.00 | 62.93 | A | C |
| ATOM | 164 | O | LYS | A | 34 | 53.447 | 64.652 | 71.514 | 1.00 | 60.74 | A | O |
| ATOM | 165 | N | GLN | A | 35 | 52.084 | 64.002 | 73.179 | 1.00 | 60.63 | A | N |
| ATOM | 166 | CA | GLN | A | 35 | 53.161 | 63.940 | 74.152 | 1.00 | 60.02 | A | C |
| ATOM | 167 | CB | GLN | A | 35 | 52.604 | 63.628 | 75.541 | 1.00 | 59.63 | A | C |
| ATOM | 168 | CG | GLN | A | 35 | 51.838 | 64.787 | 76.134 | 1.00 | 61.01 | A | C |
| ATOM | 169 | CD | GLN | A | 35 | 51.121 | 64.431 | 77.413 | 1.00 | 62.51 | A | C |
| ATOM | 170 | OE1 | GLN | A | 35 | 50.235 | 63.574 | 77.425 | 1.00 | 63.76 | A | O |
| ATOM | 171 | NE2 | GLN | A | 35 | 51.492 | 65.095 | 78.501 | 1.00 | 61.94 | A | N |
| ATOM | 172 | C | GLN | A | 35 | 54.206 | 62.901 | 73.757 | 1.00 | 61.79 | A | C |
| ATOM | 173 | O | GLN | A | 35 | 55.405 | 63.140 | 73.919 | 1.00 | 62.36 | A | O |
| ATOM | 174 | N | PHE | A | 36 | 53.767 | 61.752 | 73.239 | 1.00 | 59.61 | A | N |
| ATOM | 175 | CA | PHE | A | 36 | 54.718 | 60.721 | 72.834 | 1.00 | 56.88 | A | C |
| ATOM | 176 | CB | PHE | A | 36 | 54.021 | 59.492 | 72.246 | 1.00 | 51.57 | A | C |
| ATOM | 177 | CG | PHE | A | 36 | 53.602 | 58.485 | 73.272 | 1.00 | 47.13 | A | C |
| ATOM | 178 | CD1 | PHE | A | 36 | 52.293 | 58.447 | 73.732 | 1.00 | 45.26 | A | C |
| ATOM | 179 | CD2 | PHE | A | 36 | 54.526 | 57.594 | 73.802 | 1.00 | 45.63 | A | C |
| ATOM | 180 | CE1 | PHE | A | 36 | 51.906 | 57.540 | 74.706 | 1.00 | 45.56 | A | C |
| ATOM | 181 | CE2 | PHE | A | 36 | 54.150 | 56.674 | 74.783 | 1.00 | 46.90 | A | C |
| ATOM | 182 | CZ | PHE | A | 36 | 52.834 | 56.650 | 75.235 | 1.00 | 49.47 | A | C |
| ATOM | 183 | C | PHE | A | 36 | 55.670 | 61.280 | 71.800 | 1.00 | 59.61 | A | C |
| ATOM | 184 | O | PHE | A | 36 | 56.875 | 61.053 | 71.875 | 1.00 | 57.17 | A | O |
| ATOM | 185 | N | LEU | A | 37 | 55.124 | 62.006 | 70.829 | 1.00 | 63.39 | A | N |
| ATOM | 186 | CA | LEU | A | 37 | 55.937 | 62.597 | 69.778 | 1.00 | 69.20 | A | C |
| ATOM | 187 | CB | LEU | A | 37 | 55.054 | 63.394 | 68.819 | 1.00 | 68.34 | A | C |
| ATOM | 188 | CG | LEU | A | 37 | 54.100 | 62.560 | 67.961 | 1.00 | 64.39 | A | C |
| ATOM | 189 | CD1 | LEU | A | 37 | 53.171 | 63.475 | 67.186 | 1.00 | 66.36 | A | C |
| ATOM | 190 | CD2 | LEU | A | 37 | 54.904 | 61.684 | 67.013 | 1.00 | 64.13 | A | C |
| ATOM | 191 | C | LEU | A | 37 | 57.007 | 63.496 | 70.388 | 1.00 | 74.85 | A | C |
| ATOM | 192 | O | LEU | A | 37 | 58.158 | 63.491 | 69.947 | 1.00 | 73.98 | A | O |
| ATOM | 193 | N | ASP | A | 38 | 56.617 | 64.251 | 71.414 | 1.00 | 81.06 | A | N |
| ATOM | 194 | CA | ASP | A | 38 | 57.523 | 65.158 | 72.118 | 1.00 | 87.31 | A | C |
| ATOM | 195 | CB | ASP | A | 38 | 56.772 | 65.899 | 73.227 | 1.00 | 88.33 | A | C |
| ATOM | 196 | CG | ASP | A | 38 | 55.913 | 67.032 | 72.698 | 1.00 | 91.74 | A | C |
| ATOM | 197 | OD1 | ASP | A | 38 | 55.188 | 66.829 | 71.699 | 1.00 | 93.46 | A | O |
| ATOM | 198 | OD2 | ASP | A | 38 | 55.956 | 68.129 | 73.293 | 1.00 | 93.61 | A | O |
| ATOM | 199 | C | ASP | A | 38 | 58.703 | 64.411 | 72.732 | 1.00 | 91.19 | A | C |
| ATOM | 200 | O | ASP | A | 38 | 59.842 | 64.554 | 72.285 | 1.00 | 91.97 | A | O |
| ATOM | 201 | N | PHE | A | 39 | 58.423 | 63.616 | 73.761 | 1.00 | 95.21 | A | N |
| ATOM | 202 | CA | PHE | A | 39 | 59.459 | 62.851 | 74.443 | 1.00 | 99.65 | A | C |
| ATOM | 203 | CB | PHE | A | 39 | 58.838 | 61.961 | 75.525 | 1.00 | 102.33 | A | C |
| ATOM | 204 | CG | PHE | A | 39 | 58.248 | 62.730 | 76.677 | 1.00 | 107.69 | A | C |
| ATOM | 205 | CD1 | PHE | A | 39 | 57.180 | 63.606 | 76.476 | 1.00 | 109.32 | A | C |
| ATOM | 206 | CD2 | PHE | A | 39 | 58.775 | 62.602 | 77.960 | 1.00 | 109.15 | A | C |
| ATOM | 207 | CE1 | PHE | A | 39 | 56.645 | 64.345 | 77.538 | 1.00 | 109.49 | A | C |
| ATOM | 208 | CE2 | PHE | A | 39 | 58.249 | 63.335 | 79.027 | 1.00 | 110.03 | A | C |
| ATOM | 209 | CZ | PHE | A | 39 | 57.182 | 64.209 | 78.814 | 1.00 | 109.79 | A | C |
| ATOM | 210 | C | PHE | A | 39 | 60.259 | 62.005 | 73.462 | 1.00 | 101.82 | A | C |
| ATOM | 211 | O | PHE | A | 39 | 61.328 | 61.499 | 73.793 | 1.00 | 101.28 | A | O |
| ATOM | 212 | N | GLY | A | 40 | 59.736 | 61.864 | 72.249 | 1.00 | 104.75 | A | N |
| ATOM | 213 | CA | GLY | A | 40 | 60.418 | 61.087 | 71.231 | 1.00 | 107.83 | A | C |
| ATOM | 214 | C | GLY | A | 40 | 61.231 | 61.992 | 70.330 | 1.00 | 110.11 | A | C |
| ATOM | 215 | O | GLY | A | 40 | 62.400 | 61.724 | 70.057 | 1.00 | 110.03 | A | O |
| ATOM | 216 | N | SER | A | 41 | 60.600 | 63.068 | 69.870 | 1.00 | 113.33 | A | N |
| ATOM | 217 | CA | SER | A | 41 | 61.239 | 64.049 | 69.000 | 1.00 | 116.54 | A | C |
| ATOM | 218 | CB | SER | A | 41 | 61.884 | 65.148 | 69.853 | 1.00 | 114.81 | A | C |
| ATOM | 219 | OG | SER | A | 41 | 62.461 | 66.158 | 69.046 | 1.00 | 113.16 | A | O |
| ATOM | 220 | C | SER | A | 41 | 62.288 | 63.415 | 68.089 | 1.00 | 119.59 | A | C |
| ATOM | 221 | O | SER | A | 41 | 62.032 | 62.403 | 67.434 | 1.00 | 120.38 | A | O |
| ATOM | 222 | N | SER | A | 42 | 63.469 | 64.022 | 68.057 | 1.00 | 122.58 | A | N |
| ATOM | 223 | CA | SER | A | 42 | 64.576 | 63.537 | 67.242 | 1.00 | 124.98 | A | C |
| ATOM | 224 | CB | SER | A | 42 | 64.725 | 64.407 | 65.984 | 1.00 | 125.40 | A | C |
| ATOM | 225 | OG | SER | A | 42 | 65.764 | 63.940 | 65.139 | 1.00 | 125.63 | A | O |
| ATOM | 226 | C | SER | A | 42 | 65.845 | 63.612 | 68.088 | 1.00 | 126.04 | A | C |
| ATOM | 227 | O | SER | A | 42 | 66.792 | 62.848 | 67.884 | 1.00 | 126.34 | A | O |
| ATOM | 228 | N | ASN | A | 43 | 65.841 | 64.531 | 69.053 | 1.00 | 126.92 | A | N |
| ATOM | 229 | CA | ASN | A | 43 | 66.985 | 64.743 | 69.937 | 1.00 | 126.71 | A | C |
| ATOM | 230 | CB | ASN | A | 43 | 67.653 | 66.086 | 69.610 | 1.00 | 129.93 | A | C |
| ATOM | 231 | CG | ASN | A | 43 | 68.248 | 66.125 | 68.210 | 1.00 | 132.20 | A | C |
| ATOM | 232 | OD1 | ASN | A | 43 | 68.702 | 67.173 | 67.748 | 1.00 | 133.81 | A | O |
| ATOM | 233 | ND2 | ASN | A | 43 | 68.254 | 64.982 | 67.531 | 1.00 | 134.18 | A | N |
| ATOM | 234 | C | ASN | A | 43 | 66.630 | 64.711 | 71.426 | 1.00 | 124.43 | A | C |
| ATOM | 235 | O | ASN | A | 43 | 67.503 | 64.462 | 72.262 | 1.00 | 123.50 | A | O |
| ATOM | 236 | N | ALA | A | 44 | 65.362 | 64.975 | 71.752 | 1.00 | 121.62 | A | N |
| ATOM | 237 | CA | ALA | A | 44 | 64.897 | 64.982 | 73.142 | 1.00 | 118.00 | A | C |
| ATOM | 238 | CB | ALA | A | 44 | 63.408 | 64.654 | 73.199 | 1.00 | 116.70 | A | C |
| ATOM | 239 | C | ALA | A | 44 | 65.695 | 63.974 | 73.965 | 1.00 | 115.75 | A | C |
| ATOM | 240 | O | ALA | A | 44 | 65.607 | 62.766 | 73.739 | 1.00 | 116.22 | A | O |
| ATOM | 241 | N | CYS | A | 45 | 66.477 | 64.482 | 74.914 | 1.00 | 111.76 | A | N |
| ATOM | 242 | CA | CYS | A | 45 | 67.326 | 63.643 | 75.754 | 1.00 | 106.93 | A | C |
| ATOM | 243 | CB | CYS | A | 45 | 68.129 | 64.514 | 76.725 | 1.00 | 106.92 | A | C |
| ATOM | 244 | SG | CYS | A | 45 | 67.122 | 65.405 | 77.920 | 1.00 | 107.67 | A | S |
| ATOM | 245 | C | CYS | A | 45 | 66.600 | 62.550 | 76.534 | 1.00 | 102.98 | A | C |
| ATOM | 246 | O | CYS | A | 45 | 65.544 | 62.773 | 77.133 | 1.00 | 101.17 | A | O |
| ATOM | 247 | N | GLU | A | 46 | 67.191 | 61.362 | 76.519 | 1.00 | 98.77 | A | N |
| ATOM | 248 | CA | GLU | A | 46 | 66.638 | 60.218 | 77.223 | 1.00 | 95.60 | A | C |
| ATOM | 249 | CB | GLU | A | 46 | 67.429 | 58.958 | 76.876 | 1.00 | 94.25 | A | C |
| ATOM | 250 | CG | GLU | A | 46 | 68.923 | 59.155 | 76.773 | 1.00 | 90.49 | A | C |
| ATOM | 251 | CD | GLU | A | 46 | 69.637 | 57.890 | 76.335 | 1.00 | 90.29 | A | C |
| ATOM | 252 | OE1 | GLU | A | 46 | 69.564 | 56.880 | 77.073 | 1.00 | 88.13 | A | O |
| ATOM | 253 | OE2 | GLU | A | 46 | 70.265 | 57.905 | 75.252 | 1.00 | 89.27 | A | O |
| ATOM | 254 | C | GLU | A | 46 | 66.666 | 60.450 | 78.723 | 1.00 | 94.22 | A | C |
| ATOM | 255 | O | GLU | A | 46 | 66.108 | 59.670 | 79.498 | 1.00 | 94.72 | A | O |
| ATOM | 256 | N | LYS | A | 47 | 67.324 | 61.530 | 79.124 | 1.00 | 91.48 | A | N |
| ATOM | 257 | CA | LYS | A | 47 | 67.427 | 61.880 | 80.530 | 1.00 | 88.44 | A | C |
| ATOM | 258 | CB | LYS | A | 47 | 68.287 | 63.137 | 80.686 | 1.00 | 91.75 | A | C |
| ATOM | 259 | CG | LYS | A | 47 | 69.668 | 63.006 | 80.047 | 1.00 | 95.97 | A | C |
| ATOM | 260 | CD | LYS | A | 47 | 70.523 | 64.245 | 80.267 | 1.00 | 98.42 | A | C |
| ATOM | 261 | CE | LYS | A | 47 | 69.930 | 65.465 | 79.583 | 1.00 | 100.39 | A | C |
| ATOM | 262 | NZ | LYS | A | 47 | 70.741 | 66.691 | 79.817 | 1.00 | 101.71 | A | N |
| ATOM | 263 | C | LYS | A | 47 | 66.027 | 62.110 | 81.098 | 1.00 | 84.31 | A | C |
| ATOM | 264 | O | LYS | A | 47 | 65.682 | 61.583 | 82.159 | 1.00 | 82.77 | A | O |
| ATOM | 265 | N | THR | A | 48 | 65.224 | 62.890 | 80.379 | 1.00 | 79.49 | A | N |
| ATOM | 266 | CA | THR | A | 48 | 63.859 | 63.181 | 80.805 | 1.00 | 75.72 | A | C |
| ATOM | 267 | CB | THR | A | 48 | 63.157 | 64.124 | 79.814 | 1.00 | 75.43 | A | C |
| ATOM | 268 | OG1 | THR | A | 48 | 64.019 | 65.229 | 79.512 | 1.00 | 76.35 | A | O |
| ATOM | 269 | CG2 | THR | A | 48 | 61.857 | 64.649 | 80.413 | 1.00 | 72.73 | A | C |
| ATOM | 270 | C | THR | A | 48 | 63.073 | 61.874 | 80.873 | 1.00 | 74.33 | A | C |
| ATOM | 271 | O | THR | A | 48 | 62.349 | 61.614 | 81.838 | 1.00 | 72.95 | A | O |
| ATOM | 272 | N | SER | A | 49 | 63.231 | 61.051 | 79.839 | 1.00 | 72.69 | A | N |
| ATOM | 273 | CA | SER | A | 49 | 62.553 | 59.761 | 79.773 | 1.00 | 71.32 | A | C |
| ATOM | 274 | CB | SER | A | 49 | 62.956 | 59.015 | 78.501 | 1.00 | 71.30 | A | C |
| ATOM | 275 | OG | SER | A | 49 | 62.338 | 57.739 | 78.456 | 1.00 | 70.83 | A | O |
| ATOM | 276 | C | SER | A | 49 | 62.897 | 58.905 | 80.990 | 1.00 | 71.46 | A | C |
| ATOM | 277 | O | SER | A | 49 | 62.005 | 58.354 | 81.651 | 1.00 | 71.34 | A | O |
| ATOM | 278 | N | PHE | A | 50 | 64.195 | 58.799 | 81.277 | 1.00 | 68.88 | A | N |
| ATOM | 279 | CA | PHE | A | 50 | 64.679 | 58.022 | 82.409 | 1.00 | 65.25 | A | C |
| ATOM | 280 | CB | PHE | A | 50 | 66.203 | 58.129 | 82.518 | 1.00 | 65.81 | A | C |
| ATOM | 281 | CG | PHE | A | 50 | 66.759 | 57.490 | 83.761 | 1.00 | 67.96 | A | C |
| ATOM | 282 | CD1 | PHE | A | 50 | 66.642 | 56.115 | 83.964 | 1.00 | 66.07 | A | C |
| ATOM | 283 | CD2 | PHE | A | 50 | 67.354 | 58.267 | 84.753 | 1.00 | 67.14 | A | C |
| ATOM | 284 | CE1 | PHE | A | 50 | 67.101 | 55.524 | 85.134 | 1.00 | 66.55 | A | C |
| ATOM | 285 | CE2 | PHE | A | 50 | 67.818 | 57.685 | 85.930 | 1.00 | 68.01 | A | C |
| ATOM | 286 | CZ | PHE | A | 50 | 67.691 | 56.310 | 86.122 | 1.00 | 67.81 | A | C |
| ATOM | 287 | C | PHE | A | 50 | 64.057 | 58.470 | 83.727 | 1.00 | 64.39 | A | C |
| ATOM | 288 | O | PHE | A | 50 | 63.548 | 57.652 | 84.494 | 1.00 | 62.37 | A | O |
| ATOM | 289 | N | THR | A | 51 | 64.112 | 59.772 | 83.991 | 1.00 | 63.87 | A | N |
| ATOM | 290 | CA | THR | A | 51 | 63.563 | 60.317 | 85.227 | 1.00 | 64.20 | A | C |
| ATOM | 291 | CB | THR | A | 51 | 63.838 | 61.840 | 85.340 | 1.00 | 65.08 | A | C |
| ATOM | 292 | OG1 | THR | A | 51 | 63.127 | 62.543 | 84.314 | 1.00 | 68.31 | A | O |
| ATOM | 293 | CG2 | THR | A | 51 | 65.325 | 62.123 | 85.180 | 1.00 | 67.12 | A | C |
| ATOM | 294 | C | THR | A | 51 | 62.058 | 60.063 | 85.302 | 1.00 | 63.72 | A | C |
| ATOM | 295 | O | THR | A | 51 | 61.519 | 59.778 | 86.375 | 1.00 | 61.67 | A | O |
| ATOM | 296 | N | PHE | A | 52 | 61.386 | 60.167 | 84.157 | 1.00 | 62.03 | A | N |
| ATOM | 297 | CA | PHE | A | 52 | 59.946 | 59.936 | 84.094 | 1.00 | 61.28 | A | C |
| ATOM | 298 | CB | PHE | A | 52 | 59.389 | 60.430 | 82.745 | 1.00 | 62.52 | A | C |
| ATOM | 299 | CG | PHE | A | 52 | 57.931 | 60.085 | 82.521 | 1.00 | 61.69 | A | C |
| ATOM | 300 | CD1 | PHE | A | 52 | 56.938 | 60.619 | 83.340 | 1.00 | 60.87 | A | C |
| ATOM | 301 | CD2 | PHE | A | 52 | 57.560 | 59.194 | 81.514 | 1.00 | 59.26 | A | C |
| ATOM | 302 | CE1 | PHE | A | 52 | 55.596 | 60.267 | 83.163 | 1.00 | 61.05 | A | C |
| ATOM | 303 | CE2 | PHE | A | 52 | 56.223 | 58.836 | 81.331 | 1.00 | 58.57 | A | C |
| ATOM | 304 | CZ | PHE | A | 52 | 55.240 | 59.373 | 82.157 | 1.00 | 59.06 | A | C |
| ATOM | 305 | C | PHE | A | 52 | 59.623 | 58.448 | 84.283 | 1.00 | 60.17 | A | C |
| ATOM | 306 | O | PHE | A | 52 | 58.910 | 58.062 | 85.217 | 1.00 | 57.43 | A | O |
| ATOM | 307 | N | LEU | A | 53 | 60.165 | 57.618 | 83.396 | 1.00 | 61.13 | A | N |
| ATOM | 308 | CA | LEU | A | 53 | 59.929 | 56.178 | 83.437 | 1.00 | 64.00 | A | C |
| ATOM | 309 | CB | LEU | A | 53 | 60.714 | 55.489 | 82.315 | 1.00 | 62.87 | A | C |
| ATOM | 310 | CG | LEU | A | 53 | 60.204 | 55.805 | 80.906 | 1.00 | 62.75 | A | C |
| ATOM | 311 | CD1 | LEU | A | 53 | 61.010 | 55.058 | 79.849 | 1.00 | 62.29 | A | C |
| ATOM | 312 | CD2 | LEU | A | 53 | 58.738 | 55.414 | 80.831 | 1.00 | 63.53 | A | C |
| ATOM | 313 | C | LEU | A | 53 | 60.280 | 55.547 | 84.775 | 1.00 | 66.59 | A | C |
| ATOM | 314 | O | LEU | A | 53 | 59.657 | 54.571 | 85.198 | 1.00 | 68.41 | A | O |
| ATOM | 315 | N | ARG | A | 54 | 61.280 | 56.121 | 85.435 | 1.00 | 68.33 | A | N |
| ATOM | 316 | CA | ARG | A | 54 | 61.757 | 55.641 | 86.725 | 1.00 | 67.90 | A | C |
| ATOM | 317 | CB | ARG | A | 54 | 62.929 | 56.522 | 87.173 | 1.00 | 74.94 | A | C |
| ATOM | 318 | CG | ARG | A | 54 | 63.644 | 56.066 | 88.423 | 1.00 | 82.11 | A | C |
| ATOM | 319 | CD | ARG | A | 54 | 64.973 | 56.807 | 88.592 | 1.00 | 88.42 | A | C |
| ATOM | 320 | NE | ARG | A | 54 | 64.817 | 58.247 | 88.799 | 1.00 | 92.02 | A | N |
| ATOM | 321 | CZ | ARG | A | 54 | 65.832 | 59.086 | 88.987 | 1.00 | 93.93 | A | C |
| ATOM | 322 | NH1 | ARG | A | 54 | 67.078 | 58.631 | 88.993 | 1.00 | 94.31 | A | N |
| ATOM | 323 | NH2 | ARG | A | 54 | 65.605 | 60.379 | 89.173 | 1.00 | 94.87 | A | N |
| ATOM | 324 | C | ARG | A | 54 | 60.639 | 55.657 | 87.768 | 1.00 | 64.98 | A | C |
| ATOM | 325 | O | ARG | A | 54 | 60.618 | 54.836 | 88.684 | 1.00 | 60.54 | A | O |
| ATOM | 326 | N | GLN | A | 55 | 59.706 | 56.592 | 87.619 | 1.00 | 62.44 | A | N |
| ATOM | 327 | CA | GLN | A | 55 | 58.586 | 56.712 | 88.549 | 1.00 | 60.69 | A | C |
| ATOM | 328 | CB | GLN | A | 55 | 58.333 | 58.196 | 88.861 | 1.00 | 61.67 | A | C |
| ATOM | 329 | CG | GLN | A | 55 | 57.022 | 58.495 | 89.599 | 1.00 | 64.09 | A | C |
| ATOM | 330 | CD | GLN | A | 55 | 57.098 | 58.220 | 91.091 | 1.00 | 67.25 | A | C |
| ATOM | 331 | OE1 | GLN | A | 55 | 58.048 | 57.603 | 91.573 | 1.00 | 70.35 | A | O |
| ATOM | 332 | NE2 | GLN | A | 55 | 56.088 | 58.674 | 91.831 | 1.00 | 65.53 | A | N |
| ATOM | 333 | C | GLN | A | 55 | 57.300 | 56.067 | 88.008 | 1.00 | 58.53 | A | C |
| ATOM | 334 | O | GLN | A | 55 | 56.645 | 55.279 | 88.699 | 1.00 | 59.22 | A | O |
| ATOM | 335 | N | GLU | A | 56 | 56.953 | 56.403 | 86.770 | 1.00 | 56.74 | A | N |
| ATOM | 336 | CA | GLU | A | 56 | 55.737 | 55.902 | 86.124 | 1.00 | 52.34 | A | C |
| ATOM | 337 | CB | GLU | A | 56 | 55.644 | 56.483 | 84.703 | 1.00 | 50.80 | A | C |
| ATOM | 338 | CG | GLU | A | 56 | 54.255 | 56.430 | 84.072 | 1.00 | 47.79 | A | C |
| ATOM | 339 | CD | GLU | A | 56 | 53.168 | 56.945 | 85.001 | 1.00 | 50.55 | A | C |
| ATOM | 340 | OE1 | GLU | A | 56 | 53.429 | 57.917 | 85.750 | 1.00 | 49.87 | A | O |
| ATOM | 341 | OE2 | GLU | A | 56 | 52.048 | 56.382 | 84.977 | 1.00 | 44.40 | A | O |
| ATOM | 342 | C | GLU | A | 56 | 55.628 | 54.366 | 86.096 | 1.00 | 49.38 | A | C |
| ATOM | 343 | O | GLU | A | 56 | 54.628 | 53.802 | 86.555 | 1.00 | 48.02 | A | O |
| ATOM | 344 | N | LEU | A | 57 | 56.649 | 53.695 | 85.568 | 1.00 | 44.87 | A | N |
| ATOM | 345 | CA | LEU | A | 57 | 56.640 | 52.232 | 85.501 | 1.00 | 46.17 | A | C |
| ATOM | 346 | CB | LEU | A | 57 | 57.971 | 51.728 | 84.938 | 1.00 | 43.56 | A | C |
| ATOM | 347 | CG | LEU | A | 57 | 58.125 | 52.075 | 83.449 | 1.00 | 46.23 | A | C |
| ATOM | 348 | CD1 | LEU | A | 57 | 59.507 | 51.683 | 82.916 | 1.00 | 47.06 | A | C |
| ATOM | 349 | CD2 | LEU | A | 57 | 57.028 | 51.347 | 82.671 | 1.00 | 45.21 | A | C |
| ATOM | 350 | C | LEU | A | 57 | 56.332 | 51.592 | 86.858 | 1.00 | 47.04 | A | C |
| ATOM | 351 | O | LEU | A | 57 | 55.511 | 50.669 | 86.950 | 1.00 | 48.47 | A | O |
| ATOM | 352 | N | PRO | A | 58 | 56.986 | 52.068 | 87.932 | 1.00 | 46.47 | A | N |
| ATOM | 353 | CD | PRO | A | 58 | 58.211 | 52.894 | 87.970 | 1.00 | 48.35 | A | C |
| ATOM | 354 | CA | PRO | A | 58 | 56.702 | 51.485 | 89.246 | 1.00 | 45.93 | A | C |
| ATOM | 355 | CB | PRO | A | 58 | 57.687 | 52.212 | 90.164 | 1.00 | 47.52 | A | C |
| ATOM | 356 | CG | PRO | A | 58 | 58.876 | 52.437 | 89.249 | 1.00 | 45.74 | A | C |
| ATOM | 357 | C | PRO | A | 58 | 55.241 | 51.734 | 89.633 | 1.00 | 45.38 | A | C |
| ATOM | 358 | O | PRO | A | 58 | 54.588 | 50.881 | 90.244 | 1.00 | 45.49 | A | O |
| ATOM | 359 | N | VAL | A | 59 | 54.729 | 52.908 | 89.271 | 1.00 | 44.20 | A | N |
| ATOM | 360 | CA | VAL | A | 59 | 53.336 | 53.255 | 89.565 | 1.00 | 44.76 | A | C |
| ATOM | 361 | CB | VAL | A | 59 | 52.999 | 54.703 | 89.119 | 1.00 | 46.37 | A | C |
| ATOM | 362 | CG1 | VAL | A | 59 | 51.532 | 55.021 | 89.450 | 1.00 | 42.93 | A | C |
| ATOM | 363 | CG2 | VAL | A | 59 | 53.938 | 55.696 | 89.790 | 1.00 | 49.12 | A | C |
| ATOM | 364 | C | VAL | A | 59 | 52.358 | 52.325 | 88.839 | 1.00 | 39.87 | A | C |
| ATOM | 365 | O | VAL | A | 59 | 51.357 | 51.876 | 89.402 | 1.00 | 40.36 | A | O |
| ATOM | 366 | N | ARG | A | 60 | 52.648 | 52.038 | 87.580 | 1.00 | 40.20 | A | N |
| ATOM | 367 | CA | ARG | A | 60 | 51.756 | 51.185 | 86.803 | 1.00 | 44.97 | A | C |
| ATOM | 368 | CB | ARG | A | 60 | 52.104 | 51.295 | 85.312 | 1.00 | 43.35 | A | C |
| ATOM | 369 | CG | ARG | A | 60 | 51.859 | 52.714 | 84.757 | 1.00 | 42.05 | A | C |
| ATOM | 370 | CD | ARG | A | 60 | 51.909 | 52.806 | 83.238 | 1.00 | 40.95 | A | C |
| ATOM | 371 | NE | ARG | A | 60 | 51.706 | 54.184 | 82.793 | 1.00 | 41.24 | A | N |
| ATOM | 372 | CZ | ARG | A | 60 | 51.446 | 54.558 | 81.540 | 1.00 | 42.22 | A | C |
| ATOM | 373 | NH1 | ARG | A | 60 | 51.339 | 53.658 | 80.563 | 1.00 | 36.92 | A | N |
| ATOM | 374 | NH2 | ARG | A | 60 | 51.319 | 55.848 | 81.257 | 1.00 | 40.62 | A | N |
| ATOM | 375 | C | ARG | A | 60 | 51.783 | 49.737 | 87.280 | 1.00 | 46.25 | A | C |
| ATOM | 376 | O | ARG | A | 60 | 50.738 | 49.089 | 87.374 | 1.00 | 49.64 | A | O |
| ATOM | 377 | N | LEU | A | 61 | 52.975 | 49.245 | 87.615 | 1.00 | 47.65 | A | N |
| ATOM | 378 | CA | LEU | A | 61 | 53.137 | 47.871 | 88.092 | 1.00 | 46.75 | A | C |
| ATOM | 379 | CB | LEU | A | 61 | 54.624 | 47.560 | 88.254 | 1.00 | 44.45 | A | C |
| ATOM | 380 | CG | LEU | A | 61 | 55.393 | 47.547 | 86.936 | 1.00 | 45.23 | A | C |
| ATOM | 381 | CD1 | LEU | A | 61 | 56.876 | 47.620 | 87.208 | 1.00 | 45.26 | A | C |
| ATOM | 382 | CD2 | LEU | A | 61 | 55.042 | 46.286 | 86.150 | 1.00 | 47.04 | A | C |
| ATOM | 383 | C | LEU | A | 61 | 52.415 | 47.663 | 89.415 | 1.00 | 44.94 | A | C |
| ATOM | 384 | O | LEU | A | 61 | 51.679 | 46.680 | 89.597 | 1.00 | 39.20 | A | O |
| ATOM | 385 | N | ALA | A | 62 | 52.621 | 48.616 | 90.320 | 1.00 | 45.01 | A | N |
| ATOM | 386 | CA | ALA | A | 62 | 52.029 | 48.588 | 91.647 | 1.00 | 45.18 | A | C |
| ATOM | 387 | CB | ALA | A | 62 | 52.508 | 49.805 | 92.451 | 1.00 | 47.05 | A | C |
| ATOM | 388 | C | ALA | A | 62 | 50.515 | 48.564 | 91.594 | 1.00 | 46.06 | A | C |
| ATOM | 389 | O | ALA | A | 62 | 49.868 | 47.786 | 92.303 | 1.00 | 46.30 | A | O |
| ATOM | 390 | N | ASN | A | 63 | 49.944 | 49.427 | 90.760 | 1.00 | 47.81 | A | N |
| ATOM | 391 | CA | ASN | A | 63 | 48.491 | 49.490 | 90.633 | 1.00 | 47.43 | A | C |
| ATOM | 392 | CB | ASN | A | 63 | 48.106 | 50.566 | 89.612 | 1.00 | 49.73 | A | C |
| ATOM | 393 | CG | ASN | A | 63 | 47.813 | 51.904 | 90.265 | 1.00 | 50.76 | A | C |
| ATOM | 394 | OD1 | ASN | A | 63 | 46.696 | 52.145 | 90.733 | 1.00 | 49.24 | A | O |
| ATOM | 395 | ND2 | ASN | A | 63 | 48.820 | 52.775 | 90.317 | 1.00 | 45.47 | A | N |
| ATOM | 396 | C | ASN | A | 63 | 47.884 | 48.143 | 90.233 | 1.00 | 44.96 | A | C |
| ATOM | 397 | O | ASN | A | 63 | 46.937 | 47.655 | 90.872 | 1.00 | 44.83 | A | O |
| ATOM | 398 | N | ILE | A | 64 | 48.433 | 47.538 | 89.185 | 1.00 | 43.10 | A | N |
| ATOM | 399 | CA | ILE | A | 64 | 47.917 | 46.258 | 88.711 | 1.00 | 44.02 | A | C |
| ATOM | 400 | CB | ILE | A | 64 | 48.500 | 45.918 | 87.295 | 1.00 | 44.74 | A | C |
| ATOM | 401 | CG2 | ILE | A | 64 | 49.964 | 45.524 | 87.385 | 1.00 | 43.65 | A | C |
| ATOM | 402 | CG1 | ILE | A | 64 | 47.682 | 44.797 | 86.647 | 1.00 | 45.60 | A | C |
| ATOM | 403 | CD1 | ILE | A | 64 | 46.205 | 45.142 | 86.466 | 1.00 | 48.64 | A | C |
| ATOM | 404 | C | ILE | A | 64 | 48.192 | 45.125 | 89.724 | 1.00 | 43.50 | A | C |
| ATOM | 405 | O | ILE | A | 64 | 47.350 | 44.240 | 89.936 | 1.00 | 42.83 | A | O |
| ATOM | 406 | N | MET | A | 65 | 49.348 | 45.178 | 90.375 | 1.00 | 44.25 | A | N |
| ATOM | 407 | CA | MET | A | 65 | 49.709 | 44.161 | 91.366 | 1.00 | 44.55 | A | C |
| ATOM | 408 | CB | MET | A | 65 | 51.172 | 44.317 | 91.761 | 1.00 | 41.95 | A | C |
| ATOM | 409 | CG | MET | A | 65 | 52.094 | 43.908 | 90.640 | 1.00 | 41.15 | A | C |
| ATOM | 410 | SD | MET | A | 65 | 53.757 | 44.523 | 90.788 | 1.00 | 54.36 | A | S |
| ATOM | 411 | CE | MET | A | 65 | 54.439 | 43.302 | 91.958 | 1.00 | 46.62 | A | C |
| ATOM | 412 | C | MET | A | 65 | 48.816 | 44.264 | 92.585 | 1.00 | 49.36 | A | C |
| ATOM | 413 | O | MET | A | 65 | 48.422 | 43.247 | 93.173 | 1.00 | 49.32 | A | O |
| ATOM | 414 | N | LYS | A | 66 | 48.481 | 45.499 | 92.953 | 1.00 | 50.32 | A | N |
| ATOM | 415 | CA | LYS | A | 66 | 47.621 | 45.734 | 94.098 | 1.00 | 50.83 | A | C |
| ATOM | 416 | CB | LYS | A | 66 | 47.501 | 47.243 | 94.350 | 1.00 | 57.32 | A | C |
| ATOM | 417 | CG | LYS | A | 66 | 46.688 | 47.635 | 95.581 | 1.00 | 61.93 | A | C |
| ATOM | 418 | CD | LYS | A | 66 | 47.451 | 47.351 | 96.867 | 1.00 | 67.47 | A | C |
| ATOM | 419 | CE | LYS | A | 66 | 46.612 | 47.655 | 98.107 | 1.00 | 70.14 | A | C |
| ATOM | 420 | NZ | LYS | A | 66 | 46.300 | 49.108 | 98.265 | 1.00 | 73.81 | A | N |
| ATOM | 421 | C | LYS | A | 66 | 46.251 | 45.123 | 93.791 | 1.00 | 50.92 | A | C |
| ATOM | 422 | O | LYS | A | 66 | 45.626 | 44.502 | 94.657 | 1.00 | 48.15 | A | O |
| ATOM | 423 | N | GLU | A | 67 | 45.794 | 45.286 | 92.549 | 1.00 | 50.80 | A | N |
| ATOM | 424 | CA | GLU | A | 67 | 44.494 | 44.752 | 92.141 | 1.00 | 50.46 | A | C |
| ATOM | 425 | CB | GLU | A | 67 | 44.099 | 45.298 | 90.762 | 1.00 | 51.67 | A | C |
| ATOM | 426 | CG | GLU | A | 67 | 42.627 | 45.072 | 90.447 | 1.00 | 55.39 | A | C |
| ATOM | 427 | CD | GLU | A | 67 | 42.100 | 45.969 | 89.335 | 1.00 | 57.76 | A | C |
| ATOM | 428 | OE1 | GLU | A | 67 | 42.443 | 47.176 | 89.322 | 1.00 | 54.24 | A | O |
| ATOM | 429 | OE2 | GLU | A | 67 | 41.326 | 45.461 | 88.488 | 1.00 | 56.49 | A | O |
| ATOM | 430 | C | GLU | A | 67 | 44.509 | 43.217 | 92.118 | 1.00 | 51.00 | A | C |
| ATOM | 431 | O | GLU | A | 67 | 43.530 | 42.562 | 92.504 | 1.00 | 47.58 | A | O |
| ATOM | 432 | N | ILE | A | 68 | 45.622 | 42.652 | 91.658 | 1.00 | 49.51 | A | N |
| ATOM | 433 | CA | ILE | A | 68 | 45.769 | 41.205 | 91.611 | 1.00 | 52.26 | A | C |
| ATOM | 434 | CB | ILE | A | 68 | 47.147 | 40.810 | 91.024 | 1.00 | 50.34 | A | C |
| ATOM | 435 | CG2 | ILE | A | 68 | 47.460 | 39.366 | 91.368 | 1.00 | 47.39 | A | C |
| ATOM | 436 | CG1 | ILE | A | 68 | 47.150 | 41.018 | 89.508 | 1.00 | 42.90 | A | C |
| ATOM | 437 | CD1 | ILE | A | 68 | 48.525 | 41.010 | 88.909 | 1.00 | 43.48 | A | C |
| ATOM | 438 | C | ILE | A | 68 | 45.631 | 40.634 | 93.029 | 1.00 | 57.08 | A | C |
| ATOM | 439 | O | ILE | A | 68 | 44.763 | 39.798 | 93.287 | 1.00 | 53.53 | A | O |
| ATOM | 440 | N | ASN | A | 69 | 46.473 | 41.105 | 93.949 | 1.00 | 63.37 | A | N |
| ATOM | 441 | CA | ASN | A | 69 | 46.435 | 40.631 | 95.333 | 1.00 | 68.80 | A | C |
| ATOM | 442 | CB | ASN | A | 69 | 47.457 | 41.374 | 96.207 | 1.00 | 69.96 | A | C |
| ATOM | 443 | CG | ASN | A | 69 | 48.886 | 41.220 | 95.710 | 1.00 | 71.54 | A | C |
| ATOM | 444 | OD1 | ASN | A | 69 | 49.239 | 40.214 | 95.094 | 1.00 | 71.25 | A | O |
| ATOM | 445 | ND2 | ASN | A | 69 | 49.721 | 42.214 | 95.995 | 1.00 | 71.79 | A | N |
| ATOM | 446 | C | ASN | A | 69 | 45.061 | 40.777 | 95.972 | 1.00 | 72.86 | A | C |
| ATOM | 447 | O | ASN | A | 69 | 44.833 | 40.268 | 97.066 | 1.00 | 72.50 | A | O |
| ATOM | 448 | N | LEU | A | 70 | 44.141 | 41.461 | 95.297 | 1.00 | 78.46 | A | N |
| ATOM | 449 | CA | LEU | A | 70 | 42.808 | 41.650 | 95.858 | 1.00 | 85.41 | A | C |
| ATOM | 450 | CB | LEU | A | 70 | 42.465 | 43.139 | 95.897 | 1.00 | 86.46 | A | C |
| ATOM | 451 | CG | LEU | A | 70 | 43.342 | 43.977 | 96.829 | 1.00 | 87.98 | A | C |
| ATOM | 452 | CD1 | LEU | A | 70 | 42.820 | 45.390 | 96.838 | 1.00 | 88.29 | A | C |
| ATOM | 453 | CD2 | LEU | A | 70 | 43 329 | 43.405 | 98.243 | 1.00 | 87.84 | A | C |
| ATOM | 454 | C | LEU | A | 70 | 41.673 | 40.881 | 95.186 | 1.00 | 90.15 | A | C |
| ATOM | 455 | O | LEU | A | 70 | 40.579 | 41.414 | 94.991 | 1.00 | 91.16 | A | O |
| ATOM | 456 | N | LEU | A | 71 | 41.937 | 39.625 | 94.843 | 1.00 | 95.63 | A | N |
| ATOM | 457 | CA | LEU | A | 71 | 40.936 | 38.758 | 94.227 | 1.00 | 100.50 | A | C |
| ATOM | 458 | CB | LEU | A | 71 | 41.159 | 38.679 | 92.708 | 1.00 | 100.31 | A | C |
| ATOM | 459 | CG | LEU | A | 71 | 42.583 | 38.549 | 92.161 | 1.00 | 99.88 | A | C |
| ATOM | 460 | CD1 | LEU | A | 71 | 43.200 | 37.243 | 92.619 | 1.00 | 100.67 | A | C |
| ATOM | 461 | CD2 | LEU | A | 71 | 42.556 | 38.619 | 90.642 | 1.00 | 98.48 | A | C |
| ATOM | 462 | C | LEU | A | 71 | 41.025 | 37.372 | 94.883 | 1.00 | 103.80 | A | C |
| ATOM | 463 | O | LEU | A | 71 | 42.057 | 37.024 | 95.460 | 1.00 | 105.25 | A | O |
| ATOM | 464 | N | PRO | A | 72 | 39.945 | 36.570 | 94.808 | 1.00 | 106.19 | A | N |
| ATOM | 465 | CD | PRO | A | 72 | 38.753 | 36.814 | 93.974 | 1.00 | 106.58 | A | C |
| ATOM | 466 | CA | PRO | A | 72 | 39.889 | 35.225 | 95.395 | 1.00 | 107.91 | A | C |
| ATOM | 467 | CB | PRO | A | 72 | 38.888 | 34.520 | 94.501 | 1.00 | 107.58 | A | C |
| ATOM | 468 | CG | PRO | A | 72 | 37.884 | 35.604 | 94.278 | 1.00 | 107.19 | A | C |
| ATOM | 469 | C | PRO | A | 72 | 41.225 | 34.484 | 95.506 | 1.00 | 109.87 | A | C |
| ATOM | 470 | O | PRO | A | 72 | 42.036 | 34.489 | 94.577 | 1.00 | 110.34 | A | O |
| ATOM | 471 | N | ASP | A | 73 | 41.434 | 33.844 | 96.656 | 1.00 | 111.06 | A | N |
| ATOM | 472 | CA | ASP | A | 73 | 42.668 | 33.115 | 96.948 | 1.00 | 111.37 | A | C |
| ATOM | 473 | CB | ASP | A | 73 | 42.646 | 32.597 | 98.389 | 1.00 | 116.19 | A | C |
| ATOM | 474 | CG | ASP | A | 73 | 42.590 | 33.719 | 99.410 | 1.00 | 120.63 | A | C |
| ATOM | 475 | OD1 | ASP | A | 73 | 43.487 | 34.591 | 99.389 | 1.00 | 121.56 | A | O |
| ATOM | 476 | OD2 | ASP | A | 73 | 41.649 | 33.730 | 100.233 | 1.00 | 123.94 | A | O |
| ATOM | 477 | C | ASP | A | 73 | 42.996 | 31.965 | 96.004 | 1.00 | 108.65 | A | C |
| ATOM | 478 | O | ASP | A | 73 | 44.159 | 31.589 | 95.864 | 1.00 | 107.64 | A | O |
| ATOM | 479 | N | ARG | A | 74 | 41.981 | 31.395 | 95.366 | 1.00 | 104.80 | A | N |
| ATOM | 480 | CA | ARG | A | 74 | 42.230 | 30.303 | 94.437 | 1.00 | 100.79 | A | C |
| ATOM | 481 | CB | ARG | A | 74 | 40.934 | 29.544 | 94.144 | 1.00 | 99.37 | A | C |
| ATOM | 482 | CG | ARG | A | 74 | 39.667 | 30.361 | 94.281 | 1.00 | 96.34 | A | C |
| ATOM | 483 | CD | ARG | A | 74 | 39.503 | 31.366 | 93.165 | 1.00 | 94.13 | A | C |
| ATOM | 484 | NE | ARG | A | 74 | 38.203 | 32.019 | 93.253 | 1.00 | 92.33 | A | N |
| ATOM | 485 | CZ | ARG | A | 74 | 37.041 | 31.391 | 93.103 | 1.00 | 90.30 | A | C |
| ATOM | 486 | NH1 | ARG | A | 74 | 37.014 | 30.091 | 92.849 | 1.00 | 89.49 | A | N |
| ATOM | 487 | NH2 | ARG | A | 74 | 35.903 | 32.057 | 93.225 | 1.00 | 88.91 | A | N |
| ATOM | 488 | C | ARG | A | 74 | 42.840 | 30.847 | 93.152 | 1.00 | 99.21 | A | C |
| ATOM | 489 | O | ARG | A | 74 | 43.706 | 30.215 | 92.537 | 1.00 | 97.85 | A | O |
| ATOM | 490 | N | VAL | A | 75 | 42.394 | 32.034 | 92.755 | 1.00 | 97.19 | A | N |
| ATOM | 491 | CA | VAL | A | 75 | 42.915 | 32.661 | 91.554 | 1.00 | 94.38 | A | C |
| ATOM | 492 | CB | VAL | A | 75 | 42.096 | 33.903 | 91.158 | 1.00 | 91.44 | A | C |
| ATOM | 493 | CG1 | VAL | A | 75 | 42.492 | 34.348 | 89.766 | 1.00 | 90.68 | A | C |
| ATOM | 494 | CG2 | VAL | A | 75 | 40.610 | 33.593 | 91.209 | 1.00 | 88.63 | A | C |
| ATOM | 495 | C | VAL | A | 75 | 44.344 | 33.084 | 91.853 | 1.00 | 94.53 | A | C |
| ATOM | 496 | O | VAL | A | 75 | 45.193 | 33.104 | 90.970 | 1.00 | 95.13 | A | O |
| ATOM | 497 | N | LEU | A | 76 | 44.605 | 33.419 | 93.111 | 1.00 | 94.53 | A | N |
| ATOM | 498 | CA | LEU | A | 76 | 45.939 | 33.836 | 93.533 | 1.00 | 96.18 | A | C |
| ATOM | 499 | CB | LEU | A | 76 | 45.858 | 34.584 | 94.867 | 1.00 | 95.44 | A | C |
| ATOM | 500 | CG | LEU | A | 76 | 45.163 | 35.944 | 94.921 | 1.00 | 93.55 | A | C |
| ATOM | 501 | CD1 | LEU | A | 76 | 44.797 | 36.278 | 96.359 | 1.00 | 91.99 | A | C |
| ATOM | 502 | CD2 | LEU | A | 76 | 46.080 | 37.000 | 94.332 | 1.00 | 92.73 | A | C |
| ATOM | 503 | C | LEU | A | 76 | 46.870 | 32.633 | 93.695 | 1.00 | 97.94 | A | C |
| ATOM | 504 | O | LEU | A | 76 | 48.025 | 32.661 | 93.259 | 1.00 | 96.54 | A | O |
| ATOM | 505 | N | SER | A | 77 | 46.349 | 31.584 | 94.332 | 1.00 | 100.94 | A | N |
| ATOM | 506 | CA | SER | A | 77 | 47.105 | 30.359 | 94.594 | 1.00 | 103.08 | A | C |
| ATOM | 507 | CB | SER | A | 77 | 46.359 | 29.483 | 95.611 | 1.00 | 104.25 | A | C |
| ATOM | 508 | OG | SER | A | 77 | 46.271 | 30.117 | 96.879 | 1.00 | 106.36 | A | O |
| ATOM | 509 | C | SER | A | 77 | 47.408 | 29.540 | 93.343 | 1.00 | 102.94 | A | C |
| ATOM | 510 | O | SER | A | 77 | 48.260 | 28.648 | 93.379 | 1.00 | 103.12 | A | O |
| ATOM | 511 | N | THR | A | 78 | 46.711 | 29.834 | 92.245 | 1.00 | 102.50 | A | N |
| ATOM | 512 | CA | THR | A | 78 | 46.938 | 29.116 | 90.994 | 1.00 | 100.98 | A | C |
| ATOM | 513 | CB | THR | A | 78 | 45.992 | 29.586 | 89.879 | 1.00 | 100.58 | A | C |
| ATOM | 514 | OG1 | THR | A | 78 | 46.246 | 28.826 | 88.695 | 1.00 | 103.44 | A | O |
| ATOM | 515 | CG2 | THR | A | 78 | 46.215 | 31.039 | 89.569 | 1.00 | 100.77 | A | C |
| ATOM | 516 | C | THR | A | 78 | 48.377 | 29.345 | 90.544 | 1.00 | 99.27 | A | C |
| ATOM | 517 | O | THR | A | 78 | 48.946 | 30.414 | 90.761 | 1.00 | 99.88 | A | O |
| ATOM | 518 | N | PRO | A | 79 | 48.985 | 28.340 | 89.903 | 1.00 | 97.50 | A | N |
| ATOM | 519 | CD | PRO | A | 79 | 48.459 | 26.997 | 89.610 | 1.00 | 97.32 | A | C |
| ATOM | 520 | CA | PRO | A | 79 | 50.370 | 28.470 | 89.444 | 1.00 | 94.89 | A | C |
| ATOM | 521 | CB | PRO | A | 79 | 50.654 | 27.105 | 88.816 | 1.00 | 95.94 | A | C |
| ATOM | 522 | CG | PRO | A | 79 | 49.726 | 26.181 | 89.550 | 1.00 | 97.82 | A | C |
| ATOM | 523 | C | PRO | A | 79 | 50.604 | 29.616 | 88.461 | 1.00 | 91.82 | A | C |
| ATOM | 524 | O | PRO | A | 79 | 51.648 | 30.262 | 88.503 | 1.00 | 91.47 | A | O |
| ATOM | 525 | N | SER | A | 80 | 49.630 | 29.862 | 87.587 | 1.00 | 86.36 | A | N |
| ATOM | 526 | CA | SER | A | 80 | 49.739 | 30.907 | 86.575 | 1.00 | 79.92 | A | C |
| ATOM | 527 | CB | SER | A | 80 | 48.599 | 30.770 | 85.574 | 1.00 | 78.92 | A | C |
| ATOM | 528 | OG | SER | A | 80 | 47.363 | 30.839 | 86.246 | 1.00 | 83.21 | A | O |
| ATOM | 529 | C | SER | A | 80 | 49.747 | 32.319 | 87.151 | 1.00 | 74.72 | A | C |
| ATOM | 530 | O | SER | A | 80 | 50.746 | 33.029 | 87.057 | 1.00 | 71.56 | A | O |
| ATOM | 531 | N | VAL | A | 81 | 48.629 | 32.724 | 87.746 | 1.00 | 72.41 | A | N |
| ATOM | 532 | CA | VAL | A | 81 | 48.508 | 34.057 | 88.335 | 1.00 | 71.50 | A | C |
| ATOM | 533 | CB | VAL | A | 81 | 47.168 | 34.223 | 89.058 | 1.00 | 70.98 | A | C |
| ATOM | 534 | CG1 | VAL | A | 81 | 47.118 | 35.576 | 89.760 | 1.00 | 71.59 | A | C |
| ATOM | 535 | CG2 | VAL | A | 81 | 46.029 | 34.088 | 88.060 | 1.00 | 70.21 | A | C |
| ATOM | 536 | C | VAL | A | 81 | 49.627 | 34.354 | 89.316 | 1.00 | 71.01 | A | C |
| ATOM | 537 | O | VAL | A | 81 | 50.265 | 35.404 | 89.243 | 1.00 | 70.78 | A | O |
| ATOM | 538 | N | GLN | A | 82 | 49.867 | 33.425 | 90.237 | 1.00 | 73.12 | A | N |
| ATOM | 539 | CA | GLN | A | 82 | 50.929 | 33.601 | 91.224 | 1.00 | 71.87 | A | C |
| ATOM | 540 | CB | GLN | A | 82 | 50.946 | 32.433 | 92.218 | 1.00 | 79.04 | A | C |
| ATOM | 541 | CG | GLN | A | 82 | 51.206 | 32.845 | 93.670 | 1.00 | 87.04 | A | C |
| ATOM | 542 | CD | GLN | A | 82 | 51.619 | 31.672 | 94.549 | 1.00 | 92.89 | A | C |
| ATOM | 543 | OE1 | GLN | A | 82 | 52.758 | 31.204 | 94.485 | 1.00 | 94.42 | A | O |
| ATOM | 544 | NE2 | GLN | A | 82 | 50.689 | 31.186 | 95.367 | 1.00 | 94.61 | A | N |
| ATOM | 545 | C | GLN | A | 82 | 52.251 | 33.657 | 90.467 | 1.00 | 67.17 | A | C |
| ATOM | 546 | O | GLN | A | 82 | 53.180 | 34.370 | 90.853 | 1.00 | 66.67 | A | O |
| ATOM | 547 | N | LEU | A | 83 | 52.322 | 32.908 | 89.373 | 1.00 | 62.44 | A | N |
| ATOM | 548 | CA | LEU | A | 83 | 53.530 | 32.883 | 88.563 | 1.00 | 59.80 | A | C |
| ATOM | 549 | CB | LEU | A | 83 | 53.451 | 31.794 | 87.493 | 1.00 | 60.16 | A | C |
| ATOM | 550 | CG | LEU | A | 83 | 54.589 | 31.810 | 86.472 | 1.00 | 60.12 | A | C |
| ATOM | 551 | CD1 | LEU | A | 83 | 55.924 | 31.734 | 87.195 | 1.00 | 57.61 | A | C |
| ATOM | 552 | CD2 | LEU | A | 83 | 54.417 | 30.652 | 85.504 | 1.00 | 60.19 | A | C |
| ATOM | 553 | C | LEU | A | 83 | 53.793 | 34.221 | 87.890 | 1.00 | 59.12 | A | C |
| ATOM | 554 | O | LEU | A | 83 | 54.896 | 34.742 | 87.991 | 1.00 | 59.32 | A | O |
| ATOM | 555 | N | VAL | A | 84 | 52.805 | 34.777 | 87.186 | 1.00 | 58.16 | A | N |
| ATOM | 556 | CA | VAL | A | 84 | 53.036 | 36.068 | 86.533 | 1.00 | 56.82 | A | C |
| ATOM | 557 | CB | VAL | A | 84 | 51.881 | 36.487 | 85.561 | 1.00 | 56.83 | A | C |
| ATOM | 558 | CG1 | VAL | A | 84 | 51.750 | 35.468 | 84.443 | 1.00 | 55.09 | A | C |
| ATOM | 559 | CG2 | VAL | A | 84 | 50.571 | 36.647 | 86.311 | 1.00 | 55.62 | A | C |
| ATOM | 560 | C | VAL | A | 84 | 53.212 | 37.139 | 87.605 | 1.00 | 53.41 | A | C |
| ATOM | 561 | O | VAL | A | 84 | 54.003 | 38.064 | 87.437 | 1.00 | 51.68 | A | O |
| ATOM | 562 | N | GLN | A | 85 | 52.479 | 36.999 | 88.707 | 1.00 | 52.81 | A | N |
| ATOM | 563 | CA | GLN | A | 85 | 52.589 | 37.945 | 89.811 | 1.00 | 57.27 | A | C |
| ATOM | 564 | CB | GLN | A | 85 | 51.709 | 37.505 | 90.989 | 1.00 | 60.99 | A | C |
| ATOM | 565 | CG | GLN | A | 85 | 51.801 | 38.395 | 92.233 | 1.00 | 68.66 | A | C |
| ATOM | 566 | CD | GLN | A | 85 | 51.304 | 39.826 | 92.003 | 1.00 | 76.49 | A | C |
| ATOM | 567 | OE1 | GLN | A | 85 | 51.869 | 40.580 | 91.199 | 1.00 | 78.19 | A | O |
| ATOM | 568 | NE2 | GLN | A | 85 | 50.245 | 40.207 | 92.718 | 1.00 | 76.86 | A | N |
| ATOM | 569 | C | GLN | A | 85 | 54.048 | 38.007 | 90.251 | 1.00 | 57.92 | A | C |
| ATOM | 570 | O | GLN | A | 85 | 54.582 | 39.083 | 90.527 | 1.00 | 56.61 | A | O |
| ATOM | 571 | N | SER | A | 86 | 54.697 | 36.846 | 90.292 | 1.00 | 59.60 | A | N |
| ATOM | 572 | CA | SER | A | 86 | 56.093 | 36.785 | 90.702 | 1.00 | 62.47 | A | C |
| ATOM | 573 | CB | SER | A | 86 | 56.519 | 35.325 | 90.976 | 1.00 | 64.75 | A | C |
| ATOM | 574 | OG | SER | A | 86 | 56.451 | 34.499 | 89.824 | 1.00 | 61.84 | A | O |
| ATOM | 575 | C | SER | A | 86 | 57.003 | 37.435 | 89.665 | 1.00 | 62.14 | A | C |
| ATOM | 576 | O | SER | A | 86 | 58.093 | 37.887 | 89.991 | 1.00 | 62.77 | A | O |
| ATOM | 577 | N | TRP | A | 87 | 56.548 | 37.496 | 88.420 | 1.00 | 65.65 | A | N |
| ATOM | 578 | CA | TRP | A | 87 | 57.334 | 38.120 | 87.360 | 1.00 | 70.22 | A | C |
| ATOM | 579 | CB | TRP | A | 87 | 56.744 | 37.793 | 85.985 | 1.00 | 80.82 | A | C |
| ATOM | 580 | CG | TRP | A | 87 | 56.864 | 36.364 | 85.560 | 1.00 | 92.08 | A | C |
| ATOM | 581 | CD2 | TRP | A | 87 | 56.658 | 35.847 | 84.240 | 1.00 | 97.55 | A | C |
| ATOM | 582 | CE2 | TRP | A | 87 | 56.832 | 34.448 | 84.307 | 1.00 | 99.54 | A | C |
| ATOM | 583 | CE3 | TRP | A | 87 | 56.343 | 36.431 | 83.006 | 1.00 | 99.75 | A | C |
| ATOM | 584 | CD1 | TRP | A | 87 | 57.147 | 35.288 | 86.352 | 1.00 | 94.60 | A | C |
| ATOM | 585 | NE1 | TRP | A | 87 | 57.129 | 34.133 | 85.607 | 1.00 | 97.53 | A | N |
| ATOM | 586 | CZ2 | TRP | A | 87 | 56.700 | 33.623 | 83.186 | 1.00 | 102.14 | A | C |
| ATOM | 587 | CZ3 | TRP | A | 87 | 56.211 | 35.609 | 81.889 | 1.00 | 101.11 | A | C |
| ATOM | 588 | CH2 | TRP | A | 87 | 56.391 | 34.221 | 81.989 | 1.00 | 102.72 | A | C |
| ATOM | 589 | C | TRP | A | 87 | 57.293 | 39.628 | 87.561 | 1.00 | 67.60 | A | C |
| ATOM | 590 | O | TRP | A | 87 | 58.296 | 40.326 | 87.396 | 1.00 | 67.56 | A | O |
| ATOM | 591 | N | TYR | A | 88 | 56.113 | 40.126 | 87.907 | 1.00 | 63.40 | A | N |
| ATOM | 592 | CA | TYR | A | 88 | 55.933 | 41.551 | 88.129 | 1.00 | 61.01 | A | C |
| ATOM | 593 | CB | TYR | A | 88 | 54.446 | 41.879 | 88.289 | 1.00 | 59.69 | A | C |
| ATOM | 594 | CG | TYR | A | 88 | 53.730 | 42.055 | 86.964 | 1.00 | 59.93 | A | C |
| ATOM | 595 | CD1 | TYR | A | 88 | 53.267 | 43.307 | 86.560 | 1.00 | 57.53 | A | C |
| ATOM | 596 | CE1 | TYR | A | 88 | 52.650 | 43.481 | 85.326 | 1.00 | 59.27 | A | C |
| ATOM | 597 | CD2 | TYR | A | 88 | 53.550 | 40.976 | 86.097 | 1.00 | 58.67 | A | C |
| ATOM | 598 | CE2 | TYR | A | 88 | 52.932 | 41.140 | 84.859 | 1.00 | 58.97 | A | C |
| ATOM | 599 | CZ | TYR | A | 88 | 52.487 | 42.395 | 84.478 | 1.00 | 59.24 | A | C |
| ATOM | 600 | OH | TYR | A | 88 | 51.906 | 42.565 | 83.239 | 1.00 | 56.02 | A | O |
| ATOM | 601 | C | TYR | A | 88 | 56.711 | 42.005 | 89.352 | 1.00 | 59.04 | A | C |
| ATOM | 602 | O | TYR | A | 88 | 57.230 | 43.117 | 89.384 | 1.00 | 58.44 | A | O |
| ATOM | 603 | N | VAL | A | 89 | 56.793 | 41.143 | 90.361 | 1.00 | 60.91 | A | N |
| ATOM | 604 | CA | VAL | A | 89 | 57.535 | 41.485 | 91.571 | 1.00 | 58.90 | A | C |
| ATOM | 605 | CB | VAL | A | 89 | 57.440 | 40.373 | 92.649 | 1.00 | 59.68 | A | C |
| ATOM | 606 | CG1 | VAL | A | 89 | 58.405 | 40.682 | 93.788 | 1.00 | 59.90 | A | C |
| ATOM | 607 | CG2 | VAL | A | 89 | 56.004 | 40.278 | 93.200 | 1.00 | 53.89 | A | C |
| ATOM | 608 | C | VAL | A | 89 | 58.991 | 41.681 | 91.173 | 1.00 | 58.83 | A | C |
| ATOM | 609 | O | VAL | A | 89 | 59.626 | 42.658 | 91.560 | 1.00 | 58.45 | A | O |
| ATOM | 610 | N | GLN | A | 90 | 59.516 | 40.761 | 90.374 | 1.00 | 61.36 | A | N |
| ATOM | 611 | CA | GLN | A | 90 | 60.899 | 40.875 | 89.943 | 1.00 | 62.69 | A | C |
| ATOM | 612 | CB | GLN | A | 90 | 61.277 | 39.727 | 89.007 | 1.00 | 67.60 | A | C |
| ATOM | 613 | CG | GLN | A | 90 | 62.098 | 38.618 | 89.658 | 1.00 | 77.96 | A | C |
| ATOM | 614 | CD | GLN | A | 90 | 63.409 | 39.117 | 90.263 | 1.00 | 82.60 | A | C |
| ATOM | 615 | OE1 | GLN | A | 90 | 63.423 | 39.732 | 91.333 | 1.00 | 82.96 | A | O |
| ATOM | 616 | NE2 | GLN | A | 90 | 64.516 | 38.858 | 89.571 | 1.00 | 84.27 | A | N |
| ATOM | 617 | C | GLN | A | 90 | 61.118 | 42.198 | 89.234 | 1.00 | 60.54 | A | C |
| ATOM | 618 | O | GLN | A | 90 | 61.999 | 42.969 | 89.605 | 1.00 | 58.70 | A | O |
| ATOM | 619 | N | SER | A | 91 | 60.302 | 42.461 | 88.219 | 1.00 | 59.36 | A | N |
| ATOM | 620 | CA | SER | A | 91 | 60.419 | 43.693 | 87.443 | 1.00 | 55.79 | A | C |
| ATOM | 621 | CB | SER | A | 91 | 59.363 | 43.719 | 86.330 | 1.00 | 57.89 | A | C |
| ATOM | 622 | OG | SER | A | 91 | 59.709 | 42.835 | 85.273 | 1.00 | 57.63 | A | O |
| ATOM | 623 | C | SER | A | 91 | 60.326 | 44.970 | 88.279 | 1.00 | 52.12 | A | C |
| ATOM | 624 | O | SER | A | 91 | 61.040 | 45.938 | 88.017 | 1.00 | 44.76 | A | O |
| ATOM | 625 | N | LEU | A | 92 | 59.442 | 44.972 | 89.275 | 1.00 | 51.41 | A | N |
| ATOM | 626 | CA | LEU | A | 92 | 59.273 | 46.141 | 90.137 | 1.00 | 54.22 | A | C |
| ATOM | 627 | CB | LEU | A | 92 | 58.266 | 45.843 | 91.252 | 1.00 | 51.22 | A | C |
| ATOM | 628 | CG | LEU | A | 92 | 57.388 | 46.981 | 91.797 | 1.00 | 53.98 | A | C |
| ATOM | 629 | CD1 | LEU | A | 92 | 56.800 | 46.536 | 93.134 | 1.00 | 48.57 | A | C |
| ATOM | 630 | CD2 | LEU | A | 92 | 58.178 | 48.275 | 91.969 | 1.00 | 49.04 | A | C |
| ATOM | 631 | C | LEU | A | 92 | 60.638 | 46.454 | 90.755 | 1.00 | 57.76 | A | C |
| ATOM | 632 | O | LEU | A | 92 | 61.134 | 47.576 | 90.659 | 1.00 | 57.56 | A | O |
| ATOM | 633 | N | LEU | A | 93 | 61.240 | 45.439 | 91.375 | 1.00 | 61.82 | A | N |
| ATOM | 634 | CA | LEU | A | 93 | 62.552 | 45.563 | 92.013 | 1.00 | 65.80 | A | C |
| ATOM | 635 | CB | LEU | A | 93 | 62.974 | 44.219 | 92.616 | 1.00 | 64.08 | A | C |
| ATOM | 636 | CG | LEU | A | 93 | 61.951 | 43.549 | 93.542 | 1.00 | 66.73 | A | C |
| ATOM | 637 | CD1 | LEU | A | 93 | 62.486 | 42.213 | 94.016 | 1.00 | 67.89 | A | C |
| ATOM | 638 | CD2 | LEU | A | 93 | 61.645 | 44.450 | 94.731 | 1.00 | 66.85 | A | C |
| ATOM | 639 | C | LEU | A | 93 | 63.593 | 46.013 | 90.995 | 1.00 | 67.14 | A | C |
| ATOM | 640 | O | LEU | A | 93 | 64.351 | 46.953 | 91.243 | 1.00 | 70.10 | A | O |
| ATOM | 641 | N | ASP | A | 94 | 63.618 | 45.336 | 89.852 | 1.00 | 67.20 | A | N |
| ATOM | 642 | CA | ASP | A | 94 | 64.551 | 45.649 | 88.775 | 1.00 | 68.55 | A | C |
| ATOM | 643 | CB | ASP | A | 94 | 64.106 | 44.949 | 87.490 | 1.00 | 70.63 | A | C |
| ATOM | 644 | CG | ASP | A | 94 | 64.522 | 43.492 | 87.432 | 1.00 | 73.02 | A | C |
| ATOM | 645 | OD1 | ASP | A | 94 | 64.358 | 42.770 | 88.439 | 1.00 | 74.05 | A | O |
| ATOM | 646 | OD2 | ASP | A | 94 | 65.006 | 43.066 | 86.359 | 1.00 | 74.93 | A | O |
| ATOM | 647 | C | ASP | A | 94 | 64.666 | 47.150 | 88.504 | 1.00 | 69.75 | A | C |
| ATOM | 648 | O | ASP | A | 94 | 65.747 | 47.655 | 88.190 | 1.00 | 68.94 | A | O |
| ATOM | 649 | N | ILE | A | 95 | 63.549 | 47.860 | 88.625 | 1.00 | 71.44 | A | N |
| ATOM | 650 | CA | ILE | A | 95 | 63.530 | 49.293 | 88.358 | 1.00 | 74.91 | A | C |
| ATOM | 651 | CB | ILE | A | 95 | 62.168 | 49.712 | 87.752 | 1.00 | 77.02 | A | C |
| ATOM | 652 | CG2 | ILE | A | 95 | 62.161 | 51.206 | 87.449 | 1.00 | 77.70 | A | C |
| ATOM | 653 | CG1 | ILE | A | 95 | 61.920 | 48.919 | 86.464 | 1.00 | 76.57 | A | C |
| ATOM | 654 | CD1 | ILE | A | 95 | 60.646 | 49.286 | 85.751 | 1.00 | 78.74 | A | C |
| ATOM | 655 | C | ILE | A | 95 | 63.852 | 50.175 | 89.565 | 1.00 | 75.23 | A | C |
| ATOM | 656 | O | ILE | A | 95 | 64.471 | 51.228 | 89.410 | 1.00 | 73.73 | A | O |
| ATOM | 657 | N | MET | A | 96 | 63.436 | 49.751 | 90.757 | 1.00 | 77.30 | A | N |
| ATOM | 658 | CA | MET | A | 96 | 63.709 | 50.505 | 91.982 | 1.00 | 80.87 | A | C |
| ATOM | 659 | CB | MET | A | 96 | 63.434 | 49.633 | 93.208 | 1.00 | 77.75 | A | C |
| ATOM | 660 | CG | MET | A | 96 | 62.044 | 49.029 | 93.257 | 1.00 | 79.11 | A | C |
| ATOM | 661 | SD | MET | A | 96 | 60.775 | 50.114 | 93.956 | 1.00 | 76.62 | A | S |
| ATOM | 662 | CE | MET | A | 96 | 60.684 | 51.381 | 92.706 | 1.00 | 79.44 | A | C |
| ATOM | 663 | C | MET | A | 96 | 65.184 | 50.917 | 91.991 | 1.00 | 84.24 | A | C |
| ATOM | 664 | O | MET | A | 96 | 65.537 | 52.042 | 92.356 | 1.00 | 85.78 | A | O |
| ATOM | 665 | N | GLU | A | 97 | 66.028 | 49.976 | 91.576 | 1.00 | 86.49 | A | N |
| ATOM | 666 | CA | GLU | A | 97 | 67.478 | 50.138 | 91.509 | 1.00 | 87.76 | A | C |
| ATOM | 667 | CB | GLU | A | 97 | 68.066 | 48.999 | 90.673 | 1.00 | 91.86 | A | C |
| ATOM | 668 | CG | GLU | A | 97 | 69.582 | 48.917 | 90.683 | 1.00 | 97.49 | A | C |
| ATOM | 669 | CD | GLU | A | 97 | 70.091 | 47.759 | 91.521 | 1.00 | 100.77 | A | C |
| ATOM | 670 | OE1 | GLU | A | 97 | 71.326 | 47.650 | 91.692 | 1.00 | 102.24 | A | O |
| ATOM | 671 | OE2 | GLU | A | 97 | 69.256 | 46.959 | 92.002 | 1.00 | 100.50 | A | O |
| ATOM | 672 | C | GLU | A | 97 | 67.970 | 51.468 | 90.941 | 1.00 | 86.68 | A | C |
| ATOM | 673 | O | GLU | A | 97 | 69.120 | 51.850 | 91.162 | 1.00 | 88.00 | A | O |
| ATOM | 674 | N | PHE | A | 98 | 67.112 | 52.176 | 90.217 | 1.00 | 84.71 | A | N |
| ATOM | 675 | CA | PHE | A | 98 | 67.514 | 53.439 | 89.606 | 1.00 | 83.10 | A | C |
| ATOM | 676 | CB | PHE | A | 98 | 67.080 | 53.450 | 88.138 | 1.00 | 78.71 | A | C |
| ATOM | 677 | CG | PHE | A | 98 | 67.702 | 52.362 | 87.313 | 1.00 | 72.70 | A | C |
| ATOM | 678 | CD1 | PHE | A | 98 | 68.716 | 52.653 | 86.408 | 1.00 | 70.38 | A | C |
| ATOM | 679 | CD2 | PHE | A | 98 | 67.275 | 51.046 | 87.441 | 1.00 | 70.05 | A | C |
| ATOM | 680 | CE1 | PHE | A | 98 | 69.299 | 51.649 | 85.638 | 1.00 | 68.41 | A | C |
| ATOM | 681 | CE2 | PHE | A | 98 | 67.850 | 50.033 | 86.679 | 1.00 | 69.75 | A | C |
| ATOM | 682 | CZ | PHE | A | 98 | 68.864 | 50.334 | 85.774 | 1.00 | 68.09 | A | C |
| ATOM | 683 | C | PHE | A | 98 | 66.998 | 54.702 | 90.296 | 1.00 | 84.46 | A | C |
| ATOM | 684 | O | PHE | A | 98 | 67.400 | 55.812 | 89.942 | 1.00 | 82.34 | A | O |
| ATOM | 685 | N | LEU | A | 99 | 66.114 | 54.538 | 91.275 | 1.00 | 87.34 | A | N |
| ATOM | 686 | CA | LEU | A | 99 | 65.551 | 55.682 | 91.989 | 1.00 | 91.65 | A | C |
| ATOM | 687 | CB | LEU | A | 99 | 64.595 | 55.197 | 93.077 | 1.00 | 92.25 | A | C |
| ATOM | 688 | CG | LEU | A | 99 | 63.255 | 54.667 | 92.565 | 1.00 | 93.91 | A | C |
| ATOM | 689 | CD1 | LEU | A | 99 | 62.514 | 53.971 | 93.691 | 1.00 | 94.14 | A | C |
| ATOM | 690 | CD2 | LEU | A | 99 | 62.434 | 55.821 | 91.999 | 1.00 | 93.62 | A | C |
| ATOM | 691 | C | LEU | A | 99 | 66.603 | 56.604 | 92.603 | 1.00 | 94.50 | A | C |
| ATOM | 692 | O | LEU | A | 99 | 66.424 | 57.823 | 92.642 | 1.00 | 94.74 | A | O |
| ATOM | 693 | N | ASP | A | 100 | 67.696 | 56.017 | 93.083 | 1.00 | 96.93 | A | N |
| ATOM | 694 | CA | ASP | A | 100 | 68.778 | 56.780 | 93.695 | 1.00 | 98.09 | A | C |
| ATOM | 695 | CB | ASP | A | 100 | 69.118 | 56.199 | 95.068 | 1.00 | 100.60 | A | C |
| ATOM | 696 | CG | ASP | A | 100 | 68.113 | 56.597 | 96.131 | 1.00 | 102.73 | A | C |
| ATOM | 697 | OD1 | ASP | A | 100 | 66.896 | 56.431 | 95.900 | 1.00 | 103.65 | A | O |
| ATOM | 698 | OD2 | ASP | A | 100 | 68.541 | 57.076 | 97.203 | 1.00 | 105.14 | A | O |
| ATOM | 699 | C | ASP | A | 100 | 70.022 | 56.799 | 92.821 | 1.00 | 97.61 | A | C |
| ATOM | 700 | O | ASP | A | 100 | 71.145 | 56.737 | 93.318 | 1.00 | 97.20 | A | O |
| ATOM | 701 | N | LYS | A | 101 | 69.807 | 56.872 | 91.513 | 1.00 | 97.63 | A | N |
| ATOM | 702 | CA | LYS | A | 101 | 70.891 | 56.919 | 90.542 | 1.00 | 99.02 | A | C |
| ATOM | 703 | CB | LYS | A | 101 | 70.751 | 55.788 | 89.519 | 1.00 | 96.97 | A | C |
| ATOM | 704 | CG | LYS | A | 101 | 71.644 | 54.576 | 89.771 | 1.00 | 95.00 | A | C |
| ATOM | 705 | CD | LYS | A | 101 | 71.414 | 53.948 | 91.140 | 1.00 | 93.69 | A | C |
| ATOM | 706 | CE | LYS | A | 101 | 72.339 | 52.754 | 91.351 | 1.00 | 92.80 | A | C |
| ATOM | 707 | NZ | LYS | A | 101 | 72.234 | 52.179 | 92.722 | 1.00 | 92.49 | A | N |
| ATOM | 708 | C | LYS | A | 101 | 70.785 | 58.262 | 89.846 | 1.00 | 101.55 | A | C |
| ATOM | 709 | O | LYS | A | 101 | 69.722 | 58.876 | 89.844 | 1.00 | 101.22 | A | O |
| ATOM | 710 | N | ASP | A | 102 | 71.877 | 58.730 | 89.259 | 1.00 | 105.95 | A | N |
| ATOM | 711 | CA | ASP | A | 102 | 71.843 | 60.018 | 88.582 | 1.00 | 111.47 | A | C |
| ATOM | 712 | CB | ASP | A | 102 | 72.726 | 61.038 | 89.310 | 1.00 | 114.45 | A | C |
| ATOM | 713 | CG | ASP | A | 102 | 72.120 | 61.509 | 90.623 | 1.00 | 117.62 | A | C |
| ATOM | 714 | OD1 | ASP | A | 102 | 70.959 | 61.976 | 90.616 | 1.00 | 118.99 | A | O |
| ATOM | 715 | OD2 | ASP | A | 102 | 72.810 | 61.421 | 91.662 | 1.00 | 118.24 | A | O |
| ATOM | 716 | C | ASP | A | 102 | 72.268 | 59.947 | 87.129 | 1.00 | 113.75 | A | C |
| ATOM | 717 | O | ASP | A | 102 | 73.160 | 59.179 | 86.766 | 1.00 | 113.48 | A | O |
| ATOM | 718 | N | PRO | A | 103 | 71.615 | 60.743 | 86.270 | 1.00 | 116.29 | A | N |
| ATOM | 719 | CD | PRO | A | 103 | 70.374 | 61.498 | 86.515 | 1.00 | 115.79 | A | C |
| ATOM | 720 | CA | PRO | A | 103 | 71.954 | 60.757 | 84.848 | 1.00 | 118.91 | A | C |
| ATOM | 721 | CB | PRO | A | 103 | 70.950 | 61.745 | 84.268 | 1.00 | 117.52 | A | C |
| ATOM | 722 | CG | PRO | A | 103 | 69.753 | 61.536 | 85.141 | 1.00 | 116.39 | A | C |
| ATOM | 723 | C | PRO | A | 103 | 73.391 | 61.230 | 84.690 | 1.00 | 122.13 | A | C |
| ATOM | 724 | O | PRO | A | 103 | 74.239 | 60.950 | 85.539 | 1.00 | 121.64 | A | O |
| ATOM | 725 | N | GLU | A | 104 | 73.658 | 61.958 | 83.611 | 1.00 | 126.02 | A | N |
| ATOM | 726 | CA | GLU | A | 104 | 74.999 | 62.465 | 83.349 | 1.00 | 128.94 | A | C |
| ATOM | 727 | CB | GLU | A | 104 | 75.503 | 63.250 | 84.562 | 1.00 | 134.31 | A | C |
| ATOM | 728 | CG | GLU | A | 104 | 74.583 | 64.389 | 84.989 | 1.00 | 141.35 | A | C |
| ATOM | 729 | CD | GLU | A | 104 | 74.977 | 64.983 | 86.329 | 1.00 | 145.67 | A | C |
| ATOM | 730 | OE1 | GLU | A | 104 | 74.897 | 64.259 | 87.347 | 1.00 | 147.62 | A | O |
| ATOM | 731 | OE2 | GLU | A | 104 | 75.369 | 66.170 | 86.366 | 1.00 | 147.92 | A | O |
| ATOM | 732 | C | GLU | A | 104 | 75.933 | 61.294 | 83.048 | 1.00 | 127.92 | A | C |
| ATOM | 733 | O | GLU | A | 104 | 76.719 | 61.341 | 82.100 | 1.00 | 127.18 | A | O |
| ATOM | 734 | N | ASP | A | 105 | 75.840 | 60.244 | 83.860 | 1.00 | 126.92 | A | N |
| ATOM | 735 | CA | ASP | A | 105 | 76.658 | 59.052 | 83.674 | 1.00 | 126.09 | A | C |
| ATOM | 736 | CB | ASP | A | 105 | 76.611 | 58.171 | 84.929 | 1.00 | 126.57 | A | C |
| ATOM | 737 | CG | ASP | A | 105 | 77.567 | 56.986 | 84.857 | 1.00 | 126.95 | A | C |
| ATOM | 738 | OD1 | ASP | A | 105 | 78.767 | 57.196 | 84.574 | 1.00 | 126.65 | A | O |
| ATOM | 739 | OD2 | ASP | A | 105 | 77.121 | 55.844 | 85.094 | 1.00 | 127.59 | A | O |
| ATOM | 740 | C | ASP | A | 105 | 76.104 | 58.294 | 82.474 | 1.00 | 124.99 | A | C |
| ATOM | 741 | O | ASP | A | 105 | 75.093 | 57.598 | 82.577 | 1.00 | 124.30 | A | O |
| ATOM | 742 | N | HIS | A | 106 | 76.773 | 58.450 | 81.335 | 1.00 | 124.22 | A | N |
| ATOM | 743 | CA | HIS | A | 106 | 76.377 | 57.804 | 80.089 | 1.00 | 123.30 | A | C |
| ATOM | 744 | CB | HIS | A | 106 | 77.416 | 58.100 | 79.003 | 1.00 | 125.00 | A | C |
| ATOM | 745 | CG | HIS | A | 106 | 77.678 | 59.561 | 78.799 | 1.00 | 126.81 | A | C |
| ATOM | 746 | CD2 | HIS | A | 106 | 78.791 | 60.306 | 79.008 | 1.00 | 127.20 | A | C |
| ATOM | 747 | ND1 | HIS | A | 106 | 76.718 | 60.433 | 78.330 | 1.00 | 126.94 | A | N |
| ATOM | 748 | CE1 | HIS | A | 106 | 77.228 | 61.650 | 78.259 | 1.00 | 126.93 | A | C |
| ATOM | 749 | NE2 | HIS | A | 106 | 78.484 | 61.600 | 78.664 | 1.00 | 127.38 | A | N |
| ATOM | 750 | C | HIS | A | 106 | 76.232 | 56.297 | 80.270 | 1.00 | 122.25 | A | C |
| ATOM | 751 | O | HIS | A | 106 | 75.991 | 55.566 | 79.310 | 1.00 | 122.38 | A | O |
| ATOM | 752 | N | ARG | A | 107 | 76.382 | 55.843 | 81.509 | 1.00 | 120.57 | A | N |
| ATOM | 753 | CA | ARG | A | 107 | 76.271 | 54.430 | 81.841 | 1.00 | 118.80 | A | C |
| ATOM | 754 | CB | ARG | A | 107 | 77.441 | 54.011 | 82.731 | 1.00 | 123.23 | A | C |
| ATOM | 755 | CG | ARG | A | 107 | 78.802 | 54.214 | 82.088 | 1.00 | 128.92 | A | C |
| ATOM | 756 | CD | ARG | A | 107 | 79.924 | 54.070 | 83.102 | 1.00 | 133.98 | A | C |
| ATOM | 757 | NE | ARG | A | 107 | 81.231 | 54.285 | 82.490 | 1.00 | 138.85 | A | N |
| ATOM | 758 | CZ | ARG | A | 107 | 82.375 | 54.326 | 83.166 | 1.00 | 141.93 | A | C |
| ATOM | 759 | NH1 | ARG | A | 107 | 83.520 | 54.525 | 82.523 | 1.00 | 143.50 | A | N |
| ATOM | 760 | NH2 | ARG | A | 107 | 82.373 | 54.174 | 84.484 | 1.00 | 143.39 | A | N |
| ATOM | 761 | C | ARG | A | 107 | 74.957 | 54.165 | 82.562 | 1.00 | 114.58 | A | C |
| ATOM | 762 | O | ARG | A | 107 | 74.232 | 53.233 | 82.216 | 1.00 | 114.24 | A | O |
| ATOM | 763 | N | THR | A | 108 | 74.659 | 54.991 | 83.561 | 1.00 | 109.49 | A | N |
| ATOM | 764 | CA | THR | A | 108 | 73.430 | 54.854 | 84.337 | 1.00 | 104.95 | A | C |
| ATOM | 765 | CB | THR | A | 108 | 73.172 | 56.102 | 85.209 | 1.00 | 104.59 | A | C |
| ATOM | 766 | OG1 | THR | A | 108 | 74.290 | 56.320 | 86.075 | 1.00 | 105.86 | A | O |
| ATOM | 767 | CG2 | THR | A | 108 | 71.922 | 55.911 | 86.056 | 1.00 | 104.05 | A | C |
| ATOM | 768 | C | THR | A | 108 | 72.240 | 54.664 | 83.404 | 1.00 | 101.84 | A | C |
| ATOM | 769 | O | THR | A | 108 | 71.370 | 53.825 | 83.647 | 1.00 | 100.87 | A | O |
| ATOM | 770 | N | LEU | A | 109 | 72.214 | 55.450 | 82.332 | 1.00 | 98.19 | A | N |
| ATOM | 771 | CA | LEU | A | 109 | 71.140 | 55.379 | 81.352 | 1.00 | 94.96 | A | C |
| ATOM | 772 | CB | LEU | A | 109 | 71.231 | 56.554 | 80.374 | 1.00 | 92.17 | A | C |
| ATOM | 773 | CG | LEU | A | 109 | 71.085 | 57.968 | 80.944 | 1.00 | 89.99 | A | C |
| ATOM | 774 | CD1 | LEU | A | 109 | 71.168 | 58.985 | 79.815 | 1.00 | 87.08 | A | C |
| ATOM | 775 | CD2 | LEU | A | 109 | 69.759 | 58.095 | 81.677 | 1.00 | 88.59 | A | C |
| ATOM | 776 | C | LEU | A | 109 | 71.202 | 54.075 | 80.571 | 1.00 | 94.40 | A | C |
| ATOM | 777 | O | LEU | A | 109 | 70.204 | 53.368 | 80.442 | 1.00 | 94.09 | A | O |
| ATOM | 778 | N | SER | A | 110 | 72.384 | 53.762 | 80.054 | 1.00 | 93.92 | A | N |
| ATOM | 779 | CA | SER | A | 110 | 72.578 | 52.555 | 79.267 | 1.00 | 93.83 | A | C |
| ATOM | 780 | CB | SER | A | 110 | 74.055 | 52.403 | 78.904 | 1.00 | 93.25 | A | C |
| ATOM | 781 | OG | SER | A | 110 | 74.237 | 51.387 | 77.935 | 1.00 | 94.19 | A | O |
| ATOM | 782 | C | SER | A | 110 | 72.080 | 51.294 | 79.974 | 1.00 | 93.99 | A | C |
| ATOM | 783 | O | SER | A | 110 | 71.342 | 50.503 | 79.390 | 1.00 | 94.11 | A | O |
| ATOM | 784 | N | GLN | A | 111 | 72.480 | 51.103 | 81.227 | 1.00 | 94.50 | A | N |
| ATOM | 785 | CA | GLN | A | 111 | 72.056 | 49.923 | 81.979 | 1.00 | 94.75 | A | C |
| ATOM | 786 | CB | GLN | A | 111 | 72.872 | 49.799 | 83.274 | 1.00 | 97.27 | A | C |
| ATOM | 787 | CG | GLN | A | 111 | 73.263 | 51.125 | 83.909 | 1.00 | 99.67 | A | C |
| ATOM | 788 | CD | GLN | A | 111 | 74.307 | 50.967 | 85.001 | 1.00 | 99.00 | A | C |
| ATOM | 789 | OE1 | GLN | A | 111 | 74.023 | 50.447 | 86.082 | 1.00 | 98.52 | A | O |
| ATOM | 790 | NE2 | GLN | A | 111 | 75.527 | 51.410 | 84.718 | 1.00 | 97.58 | A | N |
| ATOM | 791 | C | GLN | A | 111 | 70.556 | 49.917 | 82.281 | 1.00 | 93.30 | A | C |
| ATOM | 792 | O | GLN | A | 111 | 69.994 | 48.893 | 82.684 | 1.00 | 91.32 | A | O |
| ATOM | 793 | N | PHE | A | 112 | 69.914 | 51.064 | 82.076 | 1.00 | 92.54 | A | N |
| ATOM | 794 | CA | PHE | A | 112 | 68.474 | 51.198 | 82.296 | 1.00 | 91.07 | A | C |
| ATOM | 795 | CB | PHE | A | 112 | 68.077 | 52.676 | 82.318 | 1.00 | 87.73 | A | C |
| ATOM | 796 | CG | PHE | A | 112 | 66.623 | 52.914 | 82.614 | 1.00 | 85.65 | A | C |
| ATOM | 797 | CD1 | PHE | A | 112 | 66.046 | 52.421 | 83.780 | 1.00 | 84.90 | A | C |
| ATOM | 798 | CD2 | PHE | A | 112 | 65.837 | 53.657 | 81.740 | 1.00 | 83.99 | A | C |
| ATOM | 799 | CE1 | PHE | A | 112 | 64.708 | 52.666 | 84.075 | 1.00 | 83.84 | A | C |
| ATOM | 800 | CE2 | PHE | A | 112 | 64.499 | 53.909 | 82.025 | 1.00 | 82.57 | A | C |
| ATOM | 801 | CZ | PHE | A | 112 | 63.933 | 53.412 | 83.196 | 1.00 | 83.03 | A | C |
| ATOM | 802 | C | PHE | A | 112 | 67.752 | 50.481 | 81.158 | 1.00 | 91.02 | A | C |
| ATOM | 803 | O | PHE | A | 112 | 66.862 | 49.663 | 81.393 | 1.00 | 90.03 | A | O |
| ATOM | 804 | N | THR | A | 113 | 68.148 | 50.793 | 79.925 | 1.00 | 90.91 | A | N |
| ATOM | 805 | CA | THR | A | 113 | 67.558 | 50.168 | 78.747 | 1.00 | 90.61 | A | C |
| ATOM | 806 | CB | THR | A | 113 | 68.214 | 50.675 | 77.445 | 1.00 | 90.47 | A | C |
| ATOM | 807 | OG1 | THR | A | 113 | 69.564 | 50.203 | 77.376 | 1.00 | 90.49 | A | O |
| ATOM | 808 | CG2 | THR | A | 113 | 68.216 | 52.197 | 77.402 | 1.00 | 89.68 | A | C |
| ATOM | 809 | C | THR | A | 113 | 67.783 | 48.663 | 78.851 | 1.00 | 90.69 | A | C |
| ATOM | 810 | O | THR | A | 113 | 66.853 | 47.873 | 78.684 | 1.00 | 90.70 | A | O |
| ATOM | 811 | N | ASP | A | 114 | 69.024 | 48.275 | 79.132 | 1.00 | 90.15 | A | N |
| ATOM | 812 | CA | ASP | A | 114 | 69.368 | 46.866 | 79.275 | 1.00 | 89.65 | A | C |
| ATOM | 813 | CB | ASP | A | 114 | 70.799 | 46.710 | 79.793 | 1.00 | 93.67 | A | C |
| ATOM | 814 | CG | ASP | A | 114 | 71.838 | 47.198 | 78.801 | 1.00 | 97.01 | A | C |
| ATOM | 815 | OD1 | ASP | A | 114 | 73.047 | 47.070 | 79.093 | 1.00 | 98.47 | A | O |
| ATOM | 816 | OD2 | ASP | A | 114 | 71.448 | 47.709 | 77.730 | 1.00 | 99.45 | A | O |
| ATOM | 817 | C | ASP | A | 114 | 68.402 | 46.231 | 80.259 | 1.00 | 86.86 | A | C |
| ATOM | 818 | O | ASP | A | 114 | 68.016 | 45.073 | 80.108 | 1.00 | 86.62 | A | O |
| ATOM | 819 | N | ALA | A | 115 | 68.016 | 47.002 | 81.270 | 1.00 | 82.96 | A | N |
| ATOM | 820 | CA | ALA | A | 115 | 67.087 | 46.524 | 82.283 | 1.00 | 79.61 | A | C |
| ATOM | 821 | CB | ALA | A | 115 | 67.107 | 47.454 | 83.486 | 1.00 | 78.62 | A | C |
| ATOM | 822 | C | ALA | A | 115 | 65.676 | 46.438 | 81.697 | 1.00 | 76.60 | A | C |
| ATOM | 823 | O | ALA | A | 115 | 64.931 | 45.499 | 81.985 | 1.00 | 76.01 | A | O |
| ATOM | 824 | N | LEU | A | 116 | 65.314 | 47.419 | 80.873 | 1.00 | 72.92 | A | N |
| ATOM | 825 | CA | LEU | A | 116 | 63.995 | 47.434 | 80.247 | 1.00 | 69.98 | A | C |
| ATOM | 826 | CB | LEU | A | 116 | 63.754 | 48.759 | 79.514 | 1.00 | 62.06 | A | C |
| ATOM | 827 | CG | LEU | A | 116 | 63.565 | 49.952 | 80.451 | 1.00 | 59.65 | A | C |
| ATOM | 828 | CD1 | LEU | A | 116 | 63.166 | 51.182 | 79.663 | 1.00 | 56.87 | A | C |
| ATOM | 829 | CD2 | LEU | A | 116 | 62.497 | 49.612 | 81.489 | 1.00 | 56.76 | A | C |
| ATOM | 830 | C | LEU | A | 116 | 63.884 | 46.273 | 79.276 | 1.00 | 70.37 | A | C |
| ATOM | 831 | O | LEU | A | 116 | 62.927 | 45.501 | 79.332 | 1.00 | 71.00 | A | O |
| ATOM | 832 | N | VAL | A | 117 | 64.869 | 46.147 | 78.393 | 1.00 | 71.35 | A | N |
| ATOM | 833 | CA | VAL | A | 117 | 64.872 | 45.060 | 77.428 | 1.00 | 73.09 | A | C |
| ATOM | 834 | CB | VAL | A | 117 | 66.165 | 45.038 | 76.598 | 1.00 | 73.32 | A | C |
| ATOM | 835 | CG1 | VAL | A | 117 | 66.174 | 43.819 | 75.685 | 1.00 | 72.75 | A | C |
| ATOM | 836 | CG2 | VAL | A | 117 | 66.272 | 46.306 | 75.773 | 1.00 | 73.26 | A | C |
| ATOM | 837 | C | VAL | A | 117 | 64.754 | 43.737 | 78.168 | 1.00 | 74.26 | A | C |
| ATOM | 838 | O | VAL | A | 117 | 64.026 | 42.840 | 77.742 | 1.00 | 76.06 | A | O |
| ATOM | 839 | N | THR | A | 118 | 65.465 | 43.619 | 79.283 | 1.00 | 74.49 | A | N |
| ATOM | 840 | CA | THR | A | 118 | 65.428 | 42.394 | 80.070 | 1.00 | 75.07 | A | C |
| ATOM | 841 | CB | THR | A | 118 | 66.443 | 42.438 | 81.236 | 1.00 | 76.52 | A | C |
| ATOM | 842 | OG1 | THR | A | 118 | 67.765 | 42.622 | 80.711 | 1.00 | 77.26 | A | O |
| ATOM | 843 | CG2 | THR | A | 118 | 66.397 | 41.138 | 82.035 | 1.00 | 73.99 | A | C |
| ATOM | 844 | C | THR | A | 118 | 64.031 | 42.160 | 80.636 | 1.00 | 73.95 | A | C |
| ATOM | 845 | O | THR | A | 118 | 63.540 | 41.029 | 80.646 | 1.00 | 73.88 | A | O |
| ATOM | 846 | N | ILE | A | 119 | 63.393 | 43.229 | 81.106 | 1.00 | 71.83 | A | N |
| ATOM | 847 | CA | ILE | A | 119 | 62.050 | 43.116 | 81.663 | 1.00 | 71.02 | A | C |
| ATOM | 848 | CB | ILE | A | 119 | 61.583 | 44.453 | 82.270 | 1.00 | 70.66 | A | C |
| ATOM | 849 | CG2 | ILE | A | 119 | 60.072 | 44.446 | 82.463 | 1.00 | 68.69 | A | C |
| ATOM | 850 | CG1 | ILE | A | 119 | 62.294 | 44.684 | 83.603 | 1.00 | 70.45 | A | C |
| ATOM | 851 | CD1 | ILE | A | 119 | 61.991 | 46.019 | 84.239 | 1.00 | 73.04 | A | C |
| ATOM | 852 | C | ILE | A | 119 | 61.047 | 42.679 | 80.601 | 1.00 | 70.44 | A | C |
| ATOM | 853 | O | ILE | A | 119 | 60.097 | 41.947 | 80.891 | 1.00 | 68.30 | A | O |
| ATOM | 854 | N | ARG | A | 120 | 61.274 | 43.124 | 79.370 | 1.00 | 70.83 | A | N |
| ATOM | 855 | CA | ARG | A | 120 | 60.389 | 42.796 | 78.261 | 1.00 | 72.67 | A | C |
| ATOM | 856 | CB | ARG | A | 120 | 60.810 | 43.579 | 77.013 | 1.00 | 73.12 | A | C |
| ATOM | 857 | CG | ARG | A | 120 | 59.922 | 43.346 | 75.803 | 1.00 | 77.77 | A | C |
| ATOM | 858 | CD | ARG | A | 120 | 60.273 | 44.296 | 74.671 | 1.00 | 80.92 | A | C |
| ATOM | 859 | NE | ARG | A | 120 | 61.681 | 44.206 | 74.294 | 1.00 | 85.39 | A | N |
| ATOM | 860 | CZ | ARG | A | 120 | 62.255 | 44.966 | 73.366 | 1.00 | 88.41 | A | C |
| ATOM | 861 | NH1 | ARG | A | 120 | 61.541 | 45.876 | 72.716 | 1.00 | 89.90 | A | N |
| ATOM | 862 | NH2 | ARG | A | 120 | 63.547 | 44.820 | 73.091 | 1.00 | 88.94 | A | N |
| ATOM | 863 | C | ARG | A | 120 | 60.353 | 41.301 | 77.954 | 1.00 | 72.68 | A | C |
| ATOM | 864 | O | ARG | A | 120 | 59.273 | 40.709 | 77.848 | 1.00 | 70.91 | A | O |
| ATOM | 865 | N | ASN | A | 121 | 61.530 | 40.693 | 77.822 | 1.00 | 72.34 | A | N |
| ATOM | 866 | CA | ASN | A | 121 | 61.628 | 39.269 | 77.517 | 1.00 | 72.09 | A | C |
| ATOM | 867 | CB | ASN | A | 121 | 63.086 | 38.872 | 77.307 | 1.00 | 74.64 | A | C |
| ATOM | 868 | CG | ASN | A | 121 | 63.864 | 39.913 | 76.543 | 1.00 | 76.84 | A | C |
| ATOM | 869 | OD1 | ASN | A | 121 | 63.398 | 40.438 | 75.528 | 1.00 | 78.86 | A | O |
| ATOM | 870 | ND2 | ASN | A | 121 | 65.063 | 40.218 | 77.023 | 1.00 | 78.22 | A | N |
| ATOM | 871 | C | ASN | A | 121 | 61.045 | 38.437 | 78.647 | 1.00 | 70.35 | A | C |
| ATOM | 872 | O | ASN | A | 121 | 60.454 | 37.380 | 78.426 | 1.00 | 72.03 | A | O |
| ATOM | 873 | N | ARG | A | 122 | 61.225 | 38.915 | 79.867 | 1.00 | 68.17 | A | N |
| ATOM | 874 | CA | ARG | A | 122 | 60.711 | 38.210 | 81.024 | 1.00 | 67.27 | A | C |
| ATOM | 875 | CB | ARG | A | 122 | 61.133 | 38.938 | 82.304 | 1.00 | 70.55 | A | C |
| ATOM | 876 | CG | ARG | A | 122 | 60.535 | 38.367 | 83.591 | 1.00 | 75.39 | A | C |
| ATOM | 877 | CD | ARG | A | 122 | 60.893 | 39.247 | 84.774 | 1.00 | 80.05 | A | C |
| ATOM | 878 | NE | ARG | A | 122 | 62.340 | 39.415 | 84.887 | 1.00 | 83.89 | A | N |
| ATOM | 879 | CZ | ARG | A | 122 | 62.937 | 40.241 | 85.741 | 1.00 | 85.41 | A | C |
| ATOM | 880 | NH1 | ARG | A | 122 | 62.217 | 40.986 | 86.566 | 1.00 | 85.60 | A | N |
| ATOM | 881 | NH2 | ARG | A | 122 | 64.261 | 40.321 | 85.766 | 1.00 | 86.40 | A | N |
| ATOM | 882 | C | ARG | A | 122 | 59.189 | 38.095 | 80.965 | 1.00 | 64.66 | A | C |
| ATOM | 883 | O | ARG | A | 122 | 58.621 | 37.121 | 81.453 | 1.00 | 63.34 | A | O |
| ATOM | 884 | N | HIS | A | 123 | 58.536 | 39.089 | 80.364 | 1.00 | 62.37 | A | N |
| ATOM | 885 | CA | HIS | A | 123 | 57.075 | 39.107 | 80.257 | 1.00 | 62.71 | A | C |
| ATOM | 886 | CB | HIS | A | 123 | 56.545 | 40.521 | 80.529 | 1.00 | 60.88 | A | C |
| ATOM | 887 | CG | HIS | A | 123 | 56.669 | 40.958 | 81.957 | 1.00 | 61.68 | A | C |
| ATOM | 888 | CD2 | HIS | A | 123 | 55.730 | 41.141 | 82.916 | 1.00 | 61.35 | A | C |
| ATOM | 889 | ND1 | HIS | A | 123 | 57.878 | 41.267 | 82.544 | 1.00 | 62.35 | A | N |
| ATOM | 890 | CE1 | HIS | A | 123 | 57.678 | 41.621 | 83.801 | 1.00 | 60.52 | A | C |
| ATOM | 891 | NE2 | HIS | A | 123 | 56.382 | 41.553 | 84.052 | 1.00 | 61.28 | A | N |
| ATOM | 892 | C | HIS | A | 123 | 56.510 | 38.624 | 78.910 | 1.00 | 62.75 | A | C |
| ATOM | 893 | O | HIS | A | 123 | 55.294 | 38.528 | 78.740 | 1.00 | 62.01 | A | O |
| ATOM | 894 | N | ASN | A | 124 | 57.391 | 38.319 | 77.963 | 1.00 | 62.70 | A | N |
| ATOM | 895 | CA | ASN | A | 124 | 56.984 | 37.867 | 76.636 | 1.00 | 63.00 | A | C |
| ATOM | 896 | CB | ASN | A | 124 | 58.207 | 37.388 | 75.848 | 1.00 | 65.89 | A | C |
| ATOM | 897 | CG | ASN | A | 124 | 58.582 | 38.335 | 74.722 | 1.00 | 71.28 | A | C |
| ATOM | 898 | OD1 | ASN | A | 124 | 57.771 | 38.618 | 73.838 | 1.00 | 73.55 | A | O |
| ATOM | 899 | ND2 | ASN | A | 124 | 59.818 | 38.830 | 74.747 | 1.00 | 72.93 | A | N |
| ATOM | 900 | C | ASN | A | 124 | 55.905 | 36.788 | 76.562 | 1.00 | 60.37 | A | C |
| ATOM | 901 | O | ASN | A | 124 | 55.287 | 36.619 | 75.520 | 1.00 | 60.43 | A | O |
| ATOM | 902 | N | ASP | A | 125 | 55.661 | 36.063 | 77.646 | 1.00 | 60.77 | A | N |
| ATOM | 903 | CA | ASP | A | 125 | 54.654 | 35.006 | 77.598 | 1.00 | 60.75 | A | C |
| ATOM | 904 | CB | ASP | A | 125 | 55.320 | 33.644 | 77.813 | 1.00 | 66.74 | A | C |
| ATOM | 905 | CG | ASP | A | 125 | 56.408 | 33.359 | 76.792 | 1.00 | 69.61 | A | C |
| ATOM | 906 | OD1 | ASP | A | 125 | 56.100 | 33.331 | 75.580 | 1.00 | 70.02 | A | O |
| ATOM | 907 | OD2 | ASP | A | 125 | 57.572 | 33.166 | 77.203 | 1.00 | 74.01 | A | O |
| ATOM | 908 | C | ASP | A | 125 | 53.524 | 35.172 | 78.601 | 1.00 | 59.06 | A | C |
| ATOM | 909 | O | ASP | A | 125 | 52.886 | 34.193 | 78.997 | 1.00 | 58.22 | A | O |
| ATOM | 910 | N | VAL | A | 126 | 53.264 | 36.410 | 79.006 | 1.00 | 55.61 | A | N |
| ATOM | 911 | CA | VAL | A | 126 | 52.207 | 36.661 | 79.969 | 1.00 | 50.82 | A | C |
| ATOM | 912 | CB | VAL | A | 126 | 52.173 | 38.149 | 80.376 | 1.00 | 52.75 | A | C |
| ATOM | 913 | CG1 | VAL | A | 126 | 51.003 | 38.405 | 81.310 | 1.00 | 52.87 | A | C |
| ATOM | 914 | CG2 | VAL | A | 126 | 53.476 | 38.529 | 81.062 | 1.00 | 51.21 | A | C |
| ATOM | 915 | C | VAL | A | 126 | 50.838 | 36.268 | 79.424 | 1.00 | 49.72 | A | C |
| ATOM | 916 | O | VAL | A | 126 | 50.005 | 35.708 | 80.147 | 1.00 | 48.10 | A | O |
| ATOM | 917 | N | VAL | A | 127 | 50.601 | 36.545 | 78.145 | 1.00 | 46.32 | A | N |
| ATOM | 918 | CA | VAL | A | 127 | 49.301 | 36.233 | 77.564 | 1.00 | 43.93 | A | C |
| ATOM | 919 | CB | VAL | A | 127 | 49.157 | 36.786 | 76.112 | 1.00 | 43.92 | A | C |
| ATOM | 920 | CG1 | VAL | A | 127 | 47.729 | 36.577 | 75.616 | 1.00 | 44.55 | A | C |
| ATOM | 921 | CG2 | VAL | A | 127 | 49.504 | 38.271 | 76.076 | 1.00 | 45.22 | A | C |
| ATOM | 922 | C | VAL | A | 127 | 49.031 | 34.736 | 77.569 | 1.00 | 41.30 | A | C |
| ATOM | 923 | O | VAL | A | 127 | 48.040 | 34.279 | 78.141 | 1.00 | 41.03 | A | O |
| ATOM | 924 | N | PRO | A | 128 | 49.904 | 33.945 | 76.930 | 1.00 | 41.66 | A | N |
| ATOM | 925 | CD | PRO | A | 128 | 51.137 | 34.270 | 76.194 | 1.00 | 38.03 | A | C |
| ATOM | 926 | CA | PRO | A | 128 | 49.652 | 32.501 | 76.935 | 1.00 | 42.26 | A | C |
| ATOM | 927 | CB | PRO | A | 128 | 50.895 | 31.931 | 76.257 | 1.00 | 44.21 | A | C |
| ATOM | 928 | CG | PRO | A | 128 | 51.328 | 33.041 | 75.355 | 1.00 | 45.87 | A | C |
| ATOM | 929 | C | PRO | A | 128 | 49.489 | 31.970 | 78.363 | 1.00 | 43.82 | A | C |
| ATOM | 930 | O | PRO | A | 128 | 48.591 | 31.166 | 78.639 | 1.00 | 44.46 | A | O |
| ATOM | 931 | N | THR | A | 129 | 50.352 | 32.433 | 79.271 | 1.00 | 44.10 | A | N |
| ATOM | 932 | CA | THR | A | 129 | 50.311 | 31.979 | 80.662 | 1.00 | 45.12 | A | C |
| ATOM | 933 | CB | THR | A | 129 | 51.487 | 32.544 | 81.496 | 1.00 | 45.90 | A | C |
| ATOM | 934 | OG1 | THR | A | 129 | 52.732 | 32.104 | 80.941 | 1.00 | 43.53 | A | O |
| ATOM | 935 | CG2 | THR | A | 129 | 51.393 | 32.058 | 82.939 | 1.00 | 45.92 | A | C |
| ATOM | 936 | C | THR | A | 129 | 49.013 | 32.354 | 81.354 | 1.00 | 46.25 | A | C |
| ATOM | 937 | O | THR | A | 129 | 48.339 | 31.493 | 81.932 | 1.00 | 44.62 | A | O |
| ATOM | 938 | N | MET | A | 130 | 48.658 | 33.635 | 81.311 | 1.00 | 46.71 | A | N |
| ATOM | 939 | CA | MET | A | 130 | 47.421 | 34.059 | 81.950 | 1.00 | 47.17 | A | C |
| ATOM | 940 | CB | MET | A | 130 | 47.213 | 35.563 | 81.772 | 1.00 | 54.07 | A | C |
| ATOM | 941 | CG | MET | A | 130 | 48.132 | 36.434 | 82.622 | 1.00 | 55.86 | A | C |
| ATOM | 942 | SD | MET | A | 130 | 47.842 | 36.271 | 84.400 | 1.00 | 59.69 | A | S |
| ATOM | 943 | CE | MET | A | 130 | 46.108 | 36.586 | 84.509 | 1.00 | 53.08 | A | C |
| ATOM | 944 | C | MET | A | 130 | 46.267 | 33.288 | 81.314 | 1.00 | 48.47 | A | C |
| ATOM | 945 | O | MET | A | 130 | 45.294 | 32.925 | 81.983 | 1.00 | 46.82 | A | O |
| ATOM | 946 | N | ALA | A | 131 | 46.384 | 33.033 | 80.013 | 1.00 | 47.16 | A | N |
| ATOM | 947 | CA | ALA | A | 131 | 45.351 | 32.294 | 79.299 | 1.00 | 48.41 | A | C |
| ATOM | 948 | CB | ALA | A | 131 | 45.678 | 32.229 | 77.803 | 1.00 | 48.01 | A | C |
| ATOM | 949 | C | ALA | A | 131 | 45.262 | 30.882 | 79.871 | 1.00 | 49.87 | A | C |
| ATOM | 950 | O | ALA | A | 131 | 44.198 | 30.257 | 79.850 | 1.00 | 47.85 | A | O |
| ATOM | 951 | N | GLN | A | 132 | 46.387 | 30.377 | 80.372 | 1.00 | 48.86 | A | N |
| ATOM | 952 | CA | GLN | A | 132 | 46.409 | 29.037 | 80.939 | 1.00 | 49.88 | A | C |
| ATOM | 953 | CB | GLN | A | 132 | 47.843 | 28.564 | 81.133 | 1.00 | 53.99 | A | C |
| ATOM | 954 | CG | GLN | A | 132 | 47.945 | 27.251 | 81.864 | 1.00 | 61.89 | A | C |
| ATOM | 955 | CD | GLN | A | 132 | 49.300 | 26.628 | 81.696 | 1.00 | 67.74 | A | C |
| ATOM | 956 | OE1 | GLN | A | 132 | 49.624 | 26.112 | 80.629 | 1.00 | 73.28 | A | O |
| ATOM | 957 | NE2 | GLN | A | 132 | 50.114 | 26.684 | 82.741 | 1.00 | 70.22 | A | N |
| ATOM | 958 | C | GLN | A | 132 | 45.654 | 28.982 | 82.259 | 1.00 | 46.84 | A | C |
| ATOM | 959 | O | GLN | A | 132 | 45.022 | 27.979 | 82.571 | 1.00 | 44.71 | A | O |
| ATOM | 960 | N | GLY | A | 133 | 45.708 | 30.067 | 83.024 | 1.00 | 46.99 | A | N |
| ATOM | 961 | CA | GLY | A | 133 | 44.993 | 30.107 | 84.285 | 1.00 | 44.36 | A | C |
| ATOM | 962 | C | GLY | A | 133 | 43.495 | 30.081 | 84.054 | 1.00 | 46.02 | A | C |
| ATOM | 963 | O | GLY | A | 133 | 42.732 | 29.529 | 84.863 | 1.00 | 45.02 | A | O |
| ATOM | 964 | N | VAL | A | 134 | 43.066 | 30.680 | 82.944 | 1.00 | 45.42 | A | N |
| ATOM | 965 | CA | VAL | A | 134 | 41.652 | 30.714 | 82.604 | 1.00 | 45.86 | A | C |
| ATOM | 966 | CB | VAL | A | 134 | 41.404 | 31.543 | 81.318 | 1.00 | 47.04 | A | C |
| ATOM | 967 | CG1 | VAL | A | 134 | 39.924 | 31.512 | 80.954 | 1.00 | 47.39 | A | C |
| ATOM | 968 | CG2 | VAL | A | 134 | 41.867 | 32.977 | 81.530 | 1.00 | 46.22 | A | C |
| ATOM | 969 | C | VAL | A | 134 | 41.128 | 29.286 | 82.401 | 1.00 | 45.32 | A | C |
| ATOM | 970 | O | VAL | A | 134 | 40.082 | 28.917 | 82.948 | 1.00 | 40.54 | A | O |
| ATOM | 971 | N | LEU | A | 135 | 41.850 | 28.484 | 81.619 | 1.00 | 45.48 | A | N |
| ATOM | 972 | CA | LEU | A | 135 | 41.428 | 27.102 | 81.396 | 1.00 | 48.70 | A | C |
| ATOM | 973 | CB | LEU | A | 135 | 42.344 | 26.395 | 80.393 | 1.00 | 49.75 | A | C |
| ATOM | 974 | CG | LEU | A | 135 | 41.970 | 24.919 | 80.179 | 1.00 | 56.33 | A | C |
| ATOM | 975 | CD1 | LEU | A | 135 | 40.455 | 24.789 | 79.893 | 1.00 | 55.02 | A | C |
| ATOM | 976 | CD2 | LEU | A | 135 | 42.804 | 24.329 | 79.036 | 1.00 | 55.30 | A | C |
| ATOM | 977 | C | LEU | A | 135 | 41.414 | 26.321 | 82.715 | 1.00 | 49.70 | A | C |
| ATOM | 978 | O | LEU | A | 135 | 40.491 | 25.543 | 82.977 | 1.00 | 48.12 | A | O |
| ATOM | 979 | N | GLU | A | 136 | 42.436 | 26.528 | 83.542 | 1.00 | 47.94 | A | N |
| ATOM | 980 | CA | GLU | A | 136 | 42.509 | 25.847 | 84.833 | 1.00 | 49.77 | A | C |
| ATOM | 981 | CB | GLU | A | 136 | 43.800 | 26.223 | 85.576 | 1.00 | 51.02 | A | C |
| ATOM | 982 | CG | GLU | A | 136 | 45.003 | 25.376 | 85.160 | 1.00 | 55.43 | A | C |
| ATOM | 983 | CD | GLU | A | 136 | 46.352 | 25.968 | 85.582 | 1.00 | 59.02 | A | C |
| ATOM | 984 | OE1 | GLU | A | 136 | 46.394 | 26.821 | 86.500 | 1.00 | 63.79 | A | O |
| ATOM | 985 | OE2 | GLU | A | 136 | 47.379 | 25.561 | 84.995 | 1.00 | 57.48 | A | O |
| ATOM | 986 | C | GLU | A | 136 | 41.298 | 26.222 | 85.674 | 1.00 | 49.61 | A | C |
| ATOM | 987 | O | GLU | A | 136 | 40.687 | 25.366 | 86.322 | 1.00 | 48.35 | A | O |
| ATOM | 988 | N | TYR | A | 137 | 40.941 | 27.502 | 85.650 | 1.00 | 47.77 | A | N |
| ATOM | 989 | CA | TYR | A | 137 | 39.804 | 27.961 | 86.425 | 1.00 | 45.88 | A | C |
| ATOM | 990 | CB | TYR | A | 137 | 39.664 | 29.481 | 86.311 | 1.00 | 48.67 | A | C |
| ATOM | 991 | CG | TYR | A | 137 | 38.539 | 30.039 | 87.151 | 1.00 | 46.25 | A | C |
| ATOM | 992 | CD1 | TYR | A | 137 | 37.204 | 29.815 | 86.806 | 1.00 | 44.03 | A | C |
| ATOM | 993 | CE1 | TYR | A | 137 | 36.165 | 30.273 | 87.615 | 1.00 | 47.21 | A | C |
| ATOM | 994 | CD2 | TYR | A | 137 | 38.810 | 30.741 | 88.324 | 1.00 | 45.51 | A | C |
| ATOM | 995 | CE2 | TYR | A | 137 | 37.782 | 31.202 | 89.138 | 1.00 | 47.43 | A | C |
| ATOM | 996 | CZ | TYR | A | 137 | 36.463 | 30.964 | 88.781 | 1.00 | 47.50 | A | C |
| ATOM | 997 | OH | TYR | A | 137 | 35.453 | 31.415 | 89.597 | 1.00 | 45.49 | A | O |
| ATOM | 998 | C | TYR | A | 137 | 38.525 | 27.275 | 85.951 | 1.00 | 45.40 | A | C |
| ATOM | 999 | O | TYR | A | 137 | 37.711 | 26.832 | 86.762 | 1.00 | 44.10 | A | O |
| ATOM | 1000 | N | LYS | A | 138 | 38.341 | 27.196 | 84.639 | 1.00 | 45.38 | A | N |
| ATOM | 1001 | CA | LYS | A | 138 | 37.157 | 26.550 | 84.084 | 1.00 | 50.23 | A | C |
| ATOM | 1002 | CB | LYS | A | 138 | 37.236 | 26.525 | 82.552 | 1.00 | 52.25 | A | C |
| ATOM | 1003 | CG | LYS | A | 138 | 36.949 | 27.868 | 81.880 | 1.00 | 59.30 | A | C |
| ATOM | 1004 | CD | LYS | A | 138 | 37.222 | 27.835 | 80.367 | 1.00 | 61.24 | A | C |
| ATOM | 1005 | CE | LYS | A | 138 | 36.316 | 26.857 | 79.610 | 1.00 | 64.11 | A | C |
| ATOM | 1006 | NZ | LYS | A | 138 | 34.880 | 27.266 | 79.573 | 1.00 | 63.73 | A | N |
| ATOM | 1007 | C | LYS | A | 138 | 37.017 | 25.119 | 84.616 | 1.00 | 50.95 | A | C |
| ATOM | 1008 | O | LYS | A | 138 | 35.939 | 24.716 | 85.063 | 1.00 | 49.28 | A | O |
| ATOM | 1009 | N | ASP | A | 139 | 38.114 | 24.363 | 84.581 | 1.00 | 50.53 | A | N |
| ATOM | 1010 | CA | ASP | A | 139 | 38.102 | 22.980 | 85.043 | 1.00 | 52.52 | A | C |
| ATOM | 1011 | CB | ASP | A | 139 | 39.346 | 22.232 | 84.542 | 1.00 | 52.18 | A | C |
| ATOM | 1012 | CG | ASP | A | 139 | 39.422 | 22.173 | 83.021 | 1.00 | 53.14 | A | C |
| ATOM | 1013 | OD1 | ASP | A | 139 | 38.352 | 22.072 | 82.371 | 1.00 | 52.02 | A | O |
| ATOM | 1014 | OD2 | ASP | A | 139 | 40.551 | 22.209 | 82.475 | 1.00 | 48.22 | A | O |
| ATOM | 1015 | C | ASP | A | 139 | 38.019 | 22.864 | 86.559 | 1.00 | 53.32 | A | C |
| ATOM | 1016 | O | ASP | A | 139 | 37.467 | 21.897 | 87.081 | 1.00 | 53.51 | A | O |
| ATOM | 1017 | N | THR | A | 140 | 38.553 | 23.857 | 87.262 | 1.00 | 55.09 | A | N |
| ATOM | 1018 | CA | THR | A | 140 | 38.544 | 23.841 | 88.719 | 1.00 | 57.15 | A | C |
| ATOM | 1019 | CB | THR | A | 140 | 39.745 | 24.623 | 89.285 | 1.00 | 57.75 | A | C |
| ATOM | 1020 | OG1 | THR | A | 140 | 40.962 | 24.052 | 88.788 | 1.00 | 60.07 | A | O |
| ATOM | 1021 | CG2 | THR | A | 140 | 39.754 | 24.551 | 90.806 | 1.00 | 58.87 | A | C |
| ATOM | 1022 | C | THR | A | 140 | 37.272 | 24.373 | 89.387 | 1.00 | 59.34 | A | C |
| ATOM | 1023 | O | THR | A | 140 | 36.827 | 23.814 | 90.389 | 1.00 | 58.72 | A | O |
| ATOM | 1024 | N | TYR | A | 141 | 36.685 | 25.440 | 88.842 | 1.00 | 60.58 | A | N |
| ATOM | 1025 | CA | TYR | A | 141 | 35.482 | 26.021 | 89.438 | 1.00 | 61.59 | A | C |
| ATOM | 1026 | CB | TYR | A | 141 | 35.807 | 27.402 | 90.006 | 1.00 | 64.10 | A | C |
| ATOM | 1027 | CG | TYR | A | 141 | 36.818 | 27.335 | 91.118 | 1.00 | 68.98 | A | C |
| ATOM | 1028 | CD1 | TYR | A | 141 | 38.154 | 27.664 | 90.897 | 1.00 | 71.17 | A | C |
| ATOM | 1029 | CE1 | TYR | A | 141 | 39.105 | 27.522 | 91.909 | 1.00 | 73.28 | A | C |
| ATOM | 1030 | CD2 | TYR | A | 141 | 36.453 | 26.866 | 92.379 | 1.00 | 71.51 | A | C |
| ATOM | 1031 | CE2 | TYR | A | 141 | 37.392 | 26.716 | 93.395 | 1.00 | 72.84 | A | C |
| ATOM | 1032 | CZ | TYR | A | 141 | 38.713 | 27.042 | 93.154 | 1.00 | 73.65 | A | C |
| ATOM | 1033 | OH | TYR | A | 141 | 39.639 | 26.861 | 94.155 | 1.00 | 77.19 | A | O |
| ATOM | 1034 | C | TYR | A | 141 | 34.231 | 26.113 | 88.566 | 1.00 | 62.05 | A | C |
| ATOM | 1035 | O | TYR | A | 141 | 33.134 | 26.316 | 89.082 | 1.00 | 60.85 | A | O |
| ATOM | 1036 | N | GLY | A | 142 | 34.381 | 25.971 | 87.254 | 1.00 | 62.16 | A | N |
| ATOM | 1037 | CA | GLY | A | 142 | 33.220 | 26.039 | 86.389 | 1.00 | 64.89 | A | C |
| ATOM | 1038 | C | GLY | A | 142 | 33.122 | 27.330 | 85.603 | 1.00 | 67.13 | A | C |
| ATOM | 1039 | O | GLY | A | 142 | 33.897 | 28.260 | 85.819 | 1.00 | 67.72 | A | O |
| ATOM | 1040 | N | ASP | A | 143 | 32.144 | 27.389 | 84.706 | 1.00 | 68.93 | A | N |
| ATOM | 1041 | CA | ASP | A | 143 | 31.942 | 28.546 | 83.842 | 1.00 | 72.08 | A | C |
| ATOM | 1042 | CB | ASP | A | 143 | 31.959 | 28.079 | 82.389 | 1.00 | 76.85 | A | C |
| ATOM | 1043 | CG | ASP | A | 143 | 31.204 | 26.774 | 82.197 | 1.00 | 82.37 | A | C |
| ATOM | 1044 | OD1 | ASP | A | 143 | 30.044 | 26.678 | 82.654 | 1.00 | 83.55 | A | O |
| ATOM | 1045 | OD2 | ASP | A | 143 | 31.772 | 25.839 | 81.590 | 1.00 | 86.68 | A | O |
| ATOM | 1046 | C | ASP | A | 143 | 30.664 | 29.348 | 84.091 | 1.00 | 71.80 | A | C |
| ATOM | 1047 | O | ASP | A | 143 | 29.932 | 29.644 | 83.150 | 1.00 | 73.42 | A | O |
| ATOM | 1048 | N | ASP | A | 144 | 30.399 | 29.712 | 85.342 | 1.00 | 72.50 | A | N |
| ATOM | 1049 | CA | ASP | A | 144 | 29.197 | 30.491 | 85.672 | 1.00 | 73.98 | A | C |
| ATOM | 1050 | CB | ASP | A | 144 | 28.903 | 30.407 | 87.172 | 1.00 | 74.17 | A | C |
| ATOM | 1051 | CG | ASP | A | 144 | 30.068 | 30.873 | 88.018 | 1.00 | 76.16 | A | C |
| ATOM | 1052 | OD1 | ASP | A | 144 | 31.112 | 30.181 | 88.022 | 1.00 | 77.72 | A | O |
| ATOM | 1053 | OD2 | ASP | A | 144 | 29.943 | 31.932 | 88.671 | 1.00 | 76.60 | A | O |
| ATOM | 1054 | C | ASP | A | 144 | 29.332 | 31.969 | 85.264 | 1.00 | 73.27 | A | C |
| ATOM | 1055 | O | ASP | A | 144 | 30.438 | 32.515 | 85.223 | 1.00 | 72.61 | A | O |
| ATOM | 1056 | N | PRO | A | 145 | 28.200 | 32.633 | 84.960 | 1.00 | 71.36 | A | N |
| ATOM | 1057 | CD | PRO | A | 145 | 26.838 | 32.073 | 84.898 | 1.00 | 70.88 | A | C |
| ATOM | 1058 | CA | PRO | A | 145 | 28.193 | 34.042 | 84.554 | 1.00 | 68.50 | A | C |
| ATOM | 1059 | CB | PRO | A | 145 | 26.705 | 34.376 | 84.526 | 1.00 | 69.14 | A | C |
| ATOM | 1060 | CG | PRO | A | 145 | 26.106 | 33.099 | 84.046 | 1.00 | 69.26 | A | C |
| ATOM | 1061 | C | PRO | A | 145 | 28.997 | 34.989 | 85.452 | 1.00 | 66.29 | A | C |
| ATOM | 1062 | O | PRO | A | 145 | 29.684 | 35.886 | 84.954 | 1.00 | 64.94 | A | O |
| ATOM | 1063 | N | VAL | A | 146 | 28.919 | 34.796 | 86.766 | 1.00 | 63.06 | A | N |
| ATOM | 1064 | CA | VAL | A | 146 | 29.655 | 35.658 | 87.686 | 1.00 | 61.21 | A | C |
| ATOM | 1065 | CB | VAL | A | 146 | 29.233 | 35.415 | 89.146 | 1.00 | 61.38 | A | C |
| ATOM | 1066 | CG1 | VAL | A | 146 | 30.168 | 36.144 | 90.105 | 1.00 | 61.66 | A | C |
| ATOM | 1067 | CG2 | VAL | A | 146 | 27.833 | 35.920 | 89.342 | 1.00 | 63.43 | A | C |
| ATOM | 1068 | C | VAL | A | 146 | 31.162 | 35.474 | 87.558 | 1.00 | 60.39 | A | C |
| ATOM | 1069 | O | VAL | A | 146 | 31.920 | 36.453 | 87.624 | 1.00 | 57.71 | A | O |
| ATOM | 1070 | N | SER | A | 147 | 31.602 | 34.228 | 87.390 | 1.00 | 56.50 | A | N |
| ATOM | 1071 | CA | SER | A | 147 | 33.027 | 33.963 | 87.219 | 1.00 | 56.86 | A | C |
| ATOM | 1072 | CB | SER | A | 147 | 33.294 | 32.454 | 87.137 | 1.00 | 56.75 | A | C |
| ATOM | 1073 | OG | SER | A | 147 | 33.210 | 31.842 | 88.413 | 1.00 | 55.85 | A | O |
| ATOM | 1074 | C | SER | A | 147 | 33.479 | 34.641 | 85.922 | 1.00 | 56.63 | A | C |
| ATOM | 1075 | O | SER | A | 147 | 34.531 | 35.281 | 85.873 | 1.00 | 53.46 | A | O |
| ATOM | 1076 | N | ASN | A | 148 | 32.667 | 34.492 | 84.877 | 1.00 | 56.49 | A | N |
| ATOM | 1077 | CA | ASN | A | 148 | 32.954 | 35.092 | 83.574 | 1.00 | 57.70 | A | C |
| ATOM | 1078 | CB | ASN | A | 148 | 31.774 | 34.845 | 82.625 | 1.00 | 60.75 | A | C |
| ATOM | 1079 | CG | ASN | A | 148 | 31.938 | 35.549 | 81.291 | 1.00 | 63.10 | A | C |
| ATOM | 1080 | OD1 | ASN | A | 148 | 32.872 | 35.268 | 80.538 | 1.00 | 62.86 | A | O |
| ATOM | 1081 | ND2 | ASN | A | 148 | 31.027 | 36.472 | 80.993 | 1.00 | 60.69 | A | N |
| ATOM | 1082 | C | ASN | A | 148 | 33.190 | 36.599 | 83.733 | 1.00 | 56.33 | A | C |
| ATOM | 1083 | O | ASN | A | 148 | 34.204 | 37.143 | 83.293 | 1.00 | 51.25 | A | O |
| ATOM | 1084 | N | GLN | A | 149 | 32.234 | 37.256 | 84.379 | 1.00 | 56.84 | A | N |
| ATOM | 1085 | CA | GLN | A | 149 | 32.286 | 38.688 | 84.625 | 1.00 | 59.29 | A | C |
| ATOM | 1086 | CB | GLN | A | 149 | 31.081 | 39.089 | 85.475 | 1.00 | 64.78 | A | C |
| ATOM | 1087 | CG | GLN | A | 149 | 30.866 | 40.581 | 85.621 | 1.00 | 73.20 | A | C |
| ATOM | 1088 | CD | GLN | A | 149 | 29.631 | 40.890 | 86.444 | 1.00 | 78.97 | A | C |
| ATOM | 1089 | OE1 | GLN | A | 149 | 28.523 | 40.446 | 86.118 | 1.00 | 79.47 | A | O |
| ATOM | 1090 | NE2 | GLN | A | 149 | 29.814 | 41.651 | 87.521 | 1.00 | 81.71 | A | N |
| ATOM | 1091 | C | GLN | A | 149 | 33.579 | 39.101 | 85.331 | 1.00 | 57.06 | A | C |
| ATOM | 1092 | O | GLN | A | 149 | 34.292 | 39.993 | 84.867 | 1.00 | 55.02 | A | O |
| ATOM | 1093 | N | ASN | A | 150 | 33.876 | 38.444 | 86.450 | 1.00 | 54.47 | A | N |
| ATOM | 1094 | CA | ASN | A | 150 | 35.075 | 38.743 | 87.231 | 1.00 | 54.41 | A | C |
| ATOM | 1095 | CB | ASN | A | 150 | 35.128 | 37.854 | 88.472 | 1.00 | 57.41 | A | C |
| ATOM | 1096 | CG | ASN | A | 150 | 34.044 | 38.191 | 89.466 | 1.00 | 59.49 | A | C |
| ATOM | 1097 | OD1 | ASN | A | 150 | 33.726 | 37.391 | 90.347 | 1.00 | 61.57 | A | O |
| ATOM | 1098 | ND2 | ASN | A | 150 | 33.472 | 39.386 | 89.337 | 1.00 | 57.67 | A | N |
| ATOM | 1099 | C | ASN | A | 150 | 36.358 | 38.573 | 86.441 | 1.00 | 50.81 | A | C |
| ATOM | 1100 | O | ASN | A | 150 | 37.276 | 39.389 | 86.543 | 1.00 | 50.75 | A | O |
| ATOM | 1101 | N | ILE | A | 151 | 36.424 | 37.501 | 85.663 | 1.00 | 48.56 | A | N |
| ATOM | 1102 | CA | ILE | A | 151 | 37.598 | 37.237 | 84.844 | 1.00 | 45.65 | A | C |
| ATOM | 1103 | CB | ILE | A | 151 | 37.534 | 35.812 | 84.230 | 1.00 | 47.36 | A | C |
| ATOM | 1104 | CG2 | ILE | A | 151 | 38.670 | 35.620 | 83.215 | 1.00 | 46.86 | A | C |
| ATOM | 1105 | CG1 | ILE | A | 151 | 37.630 | 34.764 | 85.349 | 1.00 | 49.12 | A | C |
| ATOM | 1106 | CD1 | ILE | A | 151 | 37.428 | 33.330 | 84.889 | 1.00 | 50.15 | A | C |
| ATOM | 1107 | C | ILE | A | 151 | 37.696 | 38.268 | 83.721 | 1.00 | 42.54 | A | C |
| ATOM | 1108 | O | ILE | A | 151 | 38.772 | 38.805 | 83.442 | 1.00 | 42.57 | A | O |
| ATOM | 1109 | N | GLN | A | 152 | 36.558 | 38.546 | 83.094 | 1.00 | 41.95 | A | N |
| ATOM | 1110 | CA | GLN | A | 152 | 36.485 | 39.494 | 81.982 | 1.00 | 43.24 | A | C |
| ATOM | 1111 | CB | GLN | A | 152 | 35.042 | 39.561 | 81.457 | 1.00 | 44.14 | A | C |
| ATOM | 1112 | CG | GLN | A | 152 | 34.844 | 40.260 | 80.101 | 1.00 | 50.50 | A | C |
| ATOM | 1113 | CD | GLN | A | 152 | 35.530 | 39.550 | 78.933 | 1.00 | 49.62 | A | C |
| ATOM | 1114 | OE1 | GLN | A | 152 | 35.736 | 38.331 | 78.953 | 1.00 | 49.55 | A | O |
| ATOM | 1115 | NE2 | GLN | A | 152 | 35.869 | 40.315 | 77.901 | 1.00 | 48.58 | A | N |
| ATOM | 1116 | C | GLN | A | 152 | 36.943 | 40.870 | 82.445 | 1.00 | 43.19 | A | C |
| ATOM | 1117 | O | GLN | A | 152 | 37.784 | 41.510 | 81.803 | 1.00 | 40.78 | A | O |
| ATOM | 1118 | N | TYR | A | 153 | 36.382 | 41.311 | 83.568 | 1.00 | 43.10 | A | N |
| ATOM | 1119 | CA | TYR | A | 153 | 36.716 | 42.606 | 84.137 | 1.00 | 41.94 | A | C |
| ATOM | 1120 | CB | TYR | A | 153 | 35.922 | 42.847 | 85.419 | 1.00 | 42.01 | A | C |
| ATOM | 1121 | CG | TYR | A | 153 | 36.231 | 44.181 | 86.055 | 1.00 | 48.52 | A | C |
| ATOM | 1122 | CD1 | TYR | A | 153 | 35.710 | 45.369 | 85.525 | 1.00 | 52.64 | A | C |
| ATOM | 1123 | CE1 | TYR | A | 153 | 36.040 | 46.611 | 86.074 | 1.00 | 52.59 | A | C |
| ATOM | 1124 | CD2 | TYR | A | 153 | 37.086 | 44.265 | 87.153 | 1.00 | 48.79 | A | C |
| ATOM | 1125 | CE2 | TYR | A | 153 | 37.423 | 45.495 | 87.713 | 1.00 | 51.77 | A | C |
| ATOM | 1126 | CZ | TYR | A | 153 | 36.899 | 46.664 | 87.171 | 1.00 | 55.61 | A | C |
| ATOM | 1127 | OH | TYR | A | 153 | 37.247 | 47.879 | 87.722 | 1.00 | 54.45 | A | O |
| ATOM | 1128 | C | TYR | A | 153 | 38.208 | 42.710 | 84.440 | 1.00 | 41.49 | A | C |
| ATOM | 1129 | O | TYR | A | 153 | 38.867 | 43.662 | 84.018 | 1.00 | 40.58 | A | O |
| ATOM | 1130 | N | PHE | A | 154 | 38.735 | 41.727 | 85.173 | 1.00 | 38.59 | A | N |
| ATOM | 1131 | CA | PHE | A | 154 | 40.147 | 41.716 | 85.533 | 1.00 | 34.58 | A | C |
| ATOM | 1132 | CB | PHE | A | 154 | 40.461 | 40.555 | 86.504 | 1.00 | 37.47 | A | C |
| ATOM | 1133 | CG | PHE | A | 154 | 41.943 | 40.281 | 86.655 | 1.00 | 40.74 | A | C |
| ATOM | 1134 | CD1 | PHE | A | 154 | 42.537 | 39.198 | 86.013 | 1.00 | 43.63 | A | C |
| ATOM | 1135 | CD2 | PHE | A | 154 | 42.753 | 41.145 | 87.388 | 1.00 | 40.81 | A | C |
| ATOM | 1136 | CE1 | PHE | A | 154 | 43.927 | 38.978 | 86.093 | 1.00 | 44.83 | A | C |
| ATOM | 1137 | CE2 | PHE | A | 154 | 44.134 | 40.938 | 87.477 | 1.00 | 41.40 | A | C |
| ATOM | 1138 | CZ | PHE | A | 154 | 44.725 | 39.850 | 86.824 | 1.00 | 42.65 | A | C |
| ATOM | 1139 | C | PHE | A | 154 | 41.100 | 41.621 | 84.342 | 1.00 | 31.66 | A | C |
| ATOM | 1140 | O | PHE | A | 154 | 42.048 | 42.405 | 84.230 | 1.00 | 31.83 | A | O |
| ATOM | 1141 | N | LEU | A | 155 | 40.871 | 40.648 | 83.466 | 1.00 | 33.40 | A | N |
| ATOM | 1142 | CA | LEU | A | 155 | 41.750 | 40.461 | 82.315 | 1.00 | 34.06 | A | C |
| ATOM | 1143 | CB | LEU | A | 155 | 41.387 | 39.163 | 81.569 | 1.00 | 34.17 | A | C |
| ATOM | 1144 | CG | LEU | A | 155 | 42.012 | 37.882 | 82.167 | 1.00 | 38.22 | A | C |
| ATOM | 1145 | CD1 | LEU | A | 155 | 41.640 | 36.685 | 81.308 | 1.00 | 33.39 | A | C |
| ATOM | 1146 | CD2 | LEU | A | 155 | 43.553 | 38.017 | 82.239 | 1.00 | 35.31 | A | C |
| ATOM | 1147 | C | LEU | A | 155 | 41.784 | 41.655 | 81.350 | 1.00 | 34.63 | A | C |
| ATOM | 1148 | O | LEU | A | 155 | 42.843 | 41.964 | 80.785 | 1.00 | 33.58 | A | O |
| ATOM | 1149 | N | ASP | A | 156 | 40.654 | 42.335 | 81.154 | 1.00 | 32.39 | A | N |
| ATOM | 1150 | CA | ASP | A | 156 | 40.675 | 43.500 | 80.263 | 1.00 | 34.49 | A | C |
| ATOM | 1151 | CB | ASP | A | 156 | 39.289 | 44.132 | 80.118 | 1.00 | 34.00 | A | C |
| ATOM | 1152 | CG | ASP | A | 156 | 38.427 | 43.432 | 79.086 | 1.00 | 36.89 | A | C |
| ATOM | 1153 | OD1 | ASP | A | 156 | 38.984 | 42.696 | 78.235 | 1.00 | 33.66 | A | O |
| ATOM | 1154 | OD2 | ASP | A | 156 | 37.190 | 43.638 | 79.122 | 1.00 | 38.06 | A | O |
| ATOM | 1155 | C | ASP | A | 156 | 41.627 | 44.543 | 80.851 | 1.00 | 31.65 | A | C |
| ATOM | 1156 | O | ASP | A | 156 | 42.458 | 45.116 | 80.152 | 1.00 | 30.62 | A | O |
| ATOM | 1157 | N | ARG | A | 157 | 41.496 | 44.790 | 82.149 | 1.00 | 34.06 | A | N |
| ATOM | 1158 | CA | ARG | A | 157 | 42.349 | 45.774 | 82.805 | 1.00 | 33.37 | A | C |
| ATOM | 1159 | CB | ARG | A | 157 | 41.819 | 46.033 | 84.215 | 1.00 | 33.33 | A | C |
| ATOM | 1160 | CG | ARG | A | 157 | 40.446 | 46.718 | 84.192 | 1.00 | 35.50 | A | C |
| ATOM | 1161 | CD | ARG | A | 157 | 39.713 | 46.621 | 85.528 | 1.00 | 37.87 | A | C |
| ATOM | 1162 | NE | ARG | A | 157 | 40.415 | 47.329 | 86.593 | 1.00 | 37.98 | A | N |
| ATOM | 1163 | CZ | ARG | A | 157 | 40.369 | 48.645 | 86.769 | 1.00 | 43.77 | A | C |
| ATOM | 1164 | NH1 | ARG | A | 157 | 39.641 | 49.404 | 85.950 | 1.00 | 39.56 | A | N |
| ATOM | 1165 | NH2 | ARG | A | 157 | 41.067 | 49.204 | 87.750 | 1.00 | 42.22 | A | N |
| ATOM | 1166 | C | ARG | A | 157 | 43.805 | 45.323 | 82.822 | 1.00 | 33.89 | A | C |
| ATOM | 1167 | O | ARG | A | 157 | 44.709 | 46.080 | 82.442 | 1.00 | 32.32 | A | O |
| ATOM | 1168 | N | PHE | A | 158 | 44.033 | 44.072 | 83.220 | 1.00 | 35.25 | A | N |
| ATOM | 1169 | CA | PHE | A | 158 | 45.388 | 43.546 | 83.281 | 1.00 | 34.37 | A | C |
| ATOM | 1170 | CB | PHE | A | 158 | 45.380 | 42.070 | 83.726 | 1.00 | 38.07 | A | C |
| ATOM | 1171 | CG | PHE | A | 158 | 46.744 | 41.437 | 83.714 | 1.00 | 40.20 | A | C |
| ATOM | 1172 | CD1 | PHE | A | 158 | 47.669 | 41.722 | 84.721 | 1.00 | 40.51 | A | C |
| ATOM | 1173 | CD2 | PHE | A | 158 | 47.139 | 40.627 | 82.650 | 1.00 | 40.49 | A | C |
| ATOM | 1174 | CE1 | PHE | A | 158 | 48.976 | 41.215 | 84.663 | 1.00 | 39.02 | A | C |
| ATOM | 1175 | CE2 | PHE | A | 158 | 48.444 | 40.113 | 82.581 | 1.00 | 42.02 | A | C |
| ATOM | 1176 | CZ | PHE | A | 158 | 49.364 | 40.410 | 83.591 | 1.00 | 40.16 | A | C |
| ATOM | 1177 | C | PHE | A | 158 | 46.089 | 43.660 | 81.934 | 1.00 | 35.57 | A | C |
| ATOM | 1178 | O | PHE | A | 158 | 47.219 | 44.170 | 81.846 | 1.00 | 38.01 | A | O |
| ATOM | 1179 | N | TYR | A | 159 | 45.423 | 43.181 | 80.881 | 1.00 | 34.66 | A | N |
| ATOM | 1180 | CA | TYR | A | 159 | 46.012 | 43.209 | 79.542 | 1.00 | 32.38 | A | C |
| ATOM | 1181 | CB | TYR | A | 159 | 45.165 | 42.374 | 78.566 | 1.00 | 32.69 | A | C |
| ATOM | 1182 | CG | TYR | A | 159 | 45.325 | 40.859 | 78.717 | 1.00 | 38.68 | A | C |
| ATOM | 1183 | CD1 | TYR | A | 159 | 46.584 | 40.276 | 78.932 | 1.00 | 41.10 | A | C |
| ATOM | 1184 | CE1 | TYR | A | 159 | 46.742 | 38.875 | 78.977 | 1.00 | 43.63 | A | C |
| ATOM | 1185 | CD2 | TYR | A | 159 | 44.230 | 40.007 | 78.559 | 1.00 | 38.91 | A | C |
| ATOM | 1186 | CE2 | TYR | A | 159 | 44.377 | 38.618 | 78.596 | 1.00 | 46.33 | A | C |
| ATOM | 1187 | CZ | TYR | A | 159 | 45.634 | 38.056 | 78.801 | 1.00 | 45.58 | A | C |
| ATOM | 1188 | OH | TYR | A | 159 | 45.772 | 36.685 | 78.785 | 1.00 | 50.34 | A | O |
| ATOM | 1189 | C | TYR | A | 159 | 46.196 | 44.632 | 79.007 | 1.00 | 31.36 | A | C |
| ATOM | 1190 | O | TYR | A | 159 | 47.180 | 44.919 | 78.309 | 1.00 | 25.64 | A | O |
| ATOM | 1191 | N | LEU | A | 160 | 45.248 | 45.517 | 79.315 | 1.00 | 31.90 | A | N |
| ATOM | 1192 | CA | LEU | A | 160 | 45.368 | 46.910 | 78.873 | 1.00 | 36.32 | A | C |
| ATOM | 1193 | CB | LEU | A | 160 | 44.105 | 47.706 | 79.208 | 1.00 | 33.53 | A | C |
| ATOM | 1194 | CG | LEU | A | 160 | 43.424 | 48.376 | 78.011 | 1.00 | 41.87 | A | C |
| ATOM | 1195 | CD1 | LEU | A | 160 | 42.431 | 49.392 | 78.542 | 1.00 | 44.40 | A | C |
| ATOM | 1196 | CD2 | LEU | A | 160 | 44.447 | 49.069 | 77.099 | 1.00 | 39.86 | A | C |
| ATOM | 1197 | C | LEU | A | 160 | 46.567 | 47.516 | 79.611 | 1.00 | 32.49 | A | C |
| ATOM | 1198 | O | LEU | A | 160 | 47.407 | 48.200 | 79.019 | 1.00 | 33.87 | A | O |
| ATOM | 1199 | N | SER | A | 161 | 46.644 | 47.256 | 80.911 | 1.00 | 35.59 | A | N |
| ATOM | 1200 | CA | SER | A | 161 | 47.772 | 47.758 | 81.708 | 1.00 | 36.10 | A | C |
| ATOM | 1201 | CB | SER | A | 161 | 47.721 | 47.172 | 83.124 | 1.00 | 39.75 | A | C |
| ATOM | 1202 | OG | SER | A | 161 | 48.978 | 47.287 | 83.785 | 1.00 | 44.05 | A | O |
| ATOM | 1203 | C | SER | A | 161 | 49.078 | 47.340 | 81.031 | 1.00 | 33.92 | A | C |
| ATOM | 1204 | O | SER | A | 161 | 49.993 | 48.150 | 80.858 | 1.00 | 33.03 | A | O |
| ATOM | 1205 | N | ARG | A | 162 | 49.142 | 46.073 | 80.624 | 1.00 | 31.11 | A | N |
| ATOM | 1206 | CA | ARG | A | 162 | 50.343 | 45.539 | 79.988 | 1.00 | 36.38 | A | C |
| ATOM | 1207 | CB | ARG | A | 162 | 50.236 | 44.018 | 79.773 | 1.00 | 41.85 | A | C |
| ATOM | 1208 | CG | ARG | A | 162 | 51.420 | 43.474 | 78.960 | 1.00 | 45.49 | A | C |
| ATOM | 1209 | CD | ARG | A | 162 | 51.467 | 41.969 | 78.900 | 1.00 | 47.28 | A | C |
| ATOM | 1210 | NE | ARG | A | 162 | 52.696 | 41.503 | 78.258 | 1.00 | 52.17 | A | N |
| ATOM | 1211 | CZ | ARG | A | 162 | 52.769 | 41.075 | 77.002 | 1.00 | 54.83 | A | C |
| ATOM | 1212 | NH1 | ARG | A | 162 | 51.681 | 41.051 | 76.243 | 1.00 | 55.55 | A | N |
| ATOM | 1213 | NH2 | ARG | A | 162 | 53.927 | 40.661 | 76.504 | 1.00 | 58.04 | A | N |
| ATOM | 1214 | C | ARG | A | 162 | 50.659 | 46.203 | 78.670 | 1.00 | 36.89 | A | C |
| ATOM | 1215 | O | ARG | A | 162 | 51.818 | 46.507 | 78.376 | 1.00 | 32.21 | A | O |
| ATOM | 1216 | N | ILE | A | 163 | 49.629 | 46.415 | 77.856 | 1.00 | 37.35 | A | N |
| ATOM | 1217 | CA | ILE | A | 163 | 49.854 | 47.062 | 76.581 | 1.00 | 31.77 | A | C |
| ATOM | 1218 | CB | ILE | A | 163 | 48.525 | 47.275 | 75.821 | 1.00 | 35.97 | A | C |
| ATOM | 1219 | CG2 | ILE | A | 163 | 48.749 | 48.194 | 74.627 | 1.00 | 34.82 | A | C |
| ATOM | 1220 | CG1 | ILE | A | 163 | 47.993 | 45.923 | 75.334 | 1.00 | 35.29 | A | C |
| ATOM | 1221 | CD1 | ILE | A | 163 | 46.545 | 45.941 | 74.926 | 1.00 | 41.39 | A | C |
| ATOM | 1222 | C | ILE | A | 163 | 50.546 | 48.408 | 76.805 | 1.00 | 32.09 | A | C |
| ATOM | 1223 | O | ILE | A | 163 | 51.509 | 48.739 | 76.112 | 1.00 | 32.71 | A | O |
| ATOM | 1224 | N | SER | A | 164 | 50.071 | 49.177 | 77.781 | 1.00 | 33.24 | A | N |
| ATOM | 1225 | CA | SER | A | 164 | 50.661 | 50.495 | 78.039 | 1.00 | 36.74 | A | C |
| ATOM | 1226 | CB | SER | A | 164 | 49.824 | 51.281 | 79.041 | 1.00 | 31.49 | A | C |
| ATOM | 1227 | OG | SER | A | 164 | 49.721 | 50.623 | 80.291 | 1.00 | 39.37 | A | O |
| ATOM | 1228 | C | SER | A | 164 | 52.100 | 50.424 | 78.537 | 1.00 | 41.03 | A | C |
| ATOM | 1229 | O | SER | A | 164 | 52.962 | 51.196 | 78.082 | 1.00 | 41.11 | A | O |
| ATOM | 1230 | N | ILE | A | 165 | 52.364 | 49.502 | 79.463 | 1.00 | 40.80 | A | N |
| ATOM | 1231 | CA | ILE | A | 165 | 53.718 | 49.357 | 79.984 | 1.00 | 41.50 | A | C |
| ATOM | 1232 | CB | ILE | A | 165 | 53.779 | 48.307 | 81.111 | 1.00 | 42.18 | A | C |
| ATOM | 1233 | CG2 | ILE | A | 165 | 55.237 | 48.027 | 81.482 | 1.00 | 42.29 | A | C |
| ATOM | 1234 | CG1 | ILE | A | 165 | 52.998 | 48.827 | 82.328 | 1.00 | 38.86 | A | C |
| ATOM | 1235 | CD1 | ILE | A | 165 | 52.832 | 47.833 | 83.453 | 1.00 | 37.37 | A | C |
| ATOM | 1236 | C | ILE | A | 165 | 54.655 | 48.981 | 78.848 | 1.00 | 42.52 | A | C |
| ATOM | 1237 | O | ILE | A | 165 | 55.759 | 49.522 | 78.744 | 1.00 | 42.14 | A | O |
| ATOM | 1238 | N | ARG | A | 166 | 54.217 | 48.069 | 77.981 | 1.00 | 46.07 | A | N |
| ATOM | 1239 | CA | ARG | A | 166 | 55.052 | 47.671 | 76.848 | 1.00 | 48.61 | A | C |
| ATOM | 1240 | CB | ARG | A | 166 | 54.448 | 46.464 | 76.114 | 1.00 | 55.51 | A | C |
| ATOM | 1241 | CG | ARG | A | 166 | 55.089 | 45.135 | 76.496 | 1.00 | 63.77 | A | C |
| ATOM | 1242 | CD | ARG | A | 166 | 54.313 | 43.937 | 75.971 | 1.00 | 71.57 | A | C |
| ATOM | 1243 | NE | ARG | A | 166 | 54.271 | 43.879 | 74.511 | 1.00 | 80.49 | A | N |
| ATOM | 1244 | CZ | ARG | A | 166 | 55.311 | 43.579 | 73.735 | 1.00 | 85.54 | A | C |
| ATOM | 1245 | NH1 | ARG | A | 166 | 56.492 | 43.306 | 74.279 | 1.00 | 86.96 | A | N |
| ATOM | 1246 | NH2 | ARG | A | 166 | 55.168 | 43.548 | 72.412 | 1.00 | 86.20 | A | N |
| ATOM | 1247 | C | ARG | A | 166 | 55.230 | 48.846 | 75.888 | 1.00 | 48.49 | A | C |
| ATOM | 1248 | O | ARG | A | 166 | 56.271 | 48.972 | 75.235 | 1.00 | 46.06 | A | O |
| ATOM | 1249 | N | MET | A | 167 | 54.215 | 49.708 | 75.813 | 1.00 | 48.47 | A | N |
| ATOM | 1250 | CA | MET | A | 167 | 54.270 | 50.890 | 74.952 | 1.00 | 47.20 | A | C |
| ATOM | 1251 | CB | MET | A | 167 | 52.966 | 51.700 | 75.048 | 1.00 | 48.87 | A | C |
| ATOM | 1252 | CG | MET | A | 167 | 52.242 | 51.955 | 73.729 | 1.00 | 54.71 | A | C |
| ATOM | 1253 | SD | MET | A | 167 | 53.210 | 52.652 | 72.365 | 1.00 | 49.10 | A | S |
| ATOM | 1254 | CE | MET | A | 167 | 52.510 | 54.294 | 72.210 | 1.00 | 55.94 | A | C |
| ATOM | 1255 | C | MET | A | 167 | 55.420 | 51.778 | 75.418 | 1.00 | 44.45 | A | C |
| ATOM | 1256 | O | MET | A | 167 | 56.312 | 52.120 | 74.646 | 1.00 | 44.18 | A | O |
| ATOM | 1257 | N | LEU | A | 168 | 55.385 | 52.71 | 76.687 | 1.00 | 45.67 | A | N |
| ATOM | 1258 | CA | LEU | A | 168 | 56.430 | 53.039 | 77.230 | 1.00 | 46.76 | A | C |
| ATOM | 1259 | CB | LEU | A | 168 | 56.214 | 53.253 | 78.735 | 1.00 | 42.03 | A | C |
| ATOM | 1260 | CG | LEU | A | 168 | 54.885 | 53.898 | 79.149 | 1.00 | 46.47 | A | C |
| ATOM | 1261 | CD1 | LEU | A | 168 | 54.806 | 54.013 | 80.660 | 1.00 | 43.10 | A | C |
| ATOM | 1262 | CD2 | LEU | A | 168 | 54.749 | 55.265 | 78.502 | 1.00 | 43.84 | A | C |
| ATOM | 1263 | C | LEU | A | 168 | 57.804 | 52.427 | 76.982 | 1.00 | 47.23 | A | C |
| ATOM | 1264 | O | LEU | A | 168 | 58.701 | 53.070 | 76.426 | 1.00 | 48.80 | A | O |
| ATOM | 1265 | N | ILE | A | 169 | 57.955 | 51.171 | 77.388 | 1.00 | 47.19 | A | N |
| ATOM | 1266 | CA | ILE | A | 169 | 59.212 | 50.459 | 77.222 | 1.00 | 49.91 | A | C |
| ATOM | 1267 | CB | ILE | A | 169 | 59.093 | 49.008 | 77.728 | 1.00 | 47.99 | A | C |
| ATOM | 1268 | CG2 | ILE | A | 169 | 60.305 | 48.213 | 77.309 | 1.00 | 50.36 | A | C |
| ATOM | 1269 | CG1 | ILE | A | 169 | 58.927 | 49.005 | 79.249 | 1.00 | 48.41 | A | C |
| ATOM | 1270 | CD1 | ILE | A | 169 | 58.771 | 47.621 | 79.853 | 1.00 | 48.36 | A | C |
| ATOM | 1271 | C | ILE | A | 169 | 59.688 | 50.440 | 75.774 | 1.00 | 50.77 | A | C |
| ATOM | 1272 | O | ILE | A | 169 | 60.873 | 50.626 | 75.503 | 1.00 | 51.84 | A | O |
| ATOM | 1273 | N | ASN | A | 170 | 58.768 | 50.212 | 74.843 | 1.00 | 54.27 | A | N |
| ATOM | 1274 | CA | ASN | A | 170 | 59.131 | 50.158 | 73.433 | 1.00 | 55.72 | A | C |
| ATOM | 1275 | CB | ASN | A | 170 | 57.965 | 49.644 | 72.589 | 1.00 | 60.61 | A | C |
| ATOM | 1276 | CG | ASN | A | 170 | 57.718 | 48.169 | 72.785 | 1.00 | 65.35 | A | C |
| ATOM | 1277 | OD1 | ASN | A | 170 | 58.656 | 47.370 | 72.799 | 1.00 | 69.13 | A | O |
| ATOM | 1278 | ND2 | ASN | A | 170 | 56.451 | 47.792 | 72.924 | 1.00 | 68.21 | A | N |
| ATOM | 1279 | C | ASN | A | 170 | 59.573 | 51.502 | 72.891 | 1.00 | 56.92 | A | C |
| ATOM | 1280 | O | ASN | A | 170 | 60.507 | 51.575 | 72.095 | 1.00 | 57.82 | A | O |
| ATOM | 1281 | N | GLN | A | 171 | 58.900 | 52.568 | 73.304 | 1.00 | 56.11 | A | N |
| ATOM | 1282 | CA | GLN | A | 171 | 59.277 | 53.885 | 72.820 | 1.00 | 59.79 | A | C |
| ATOM | 1283 | CB | GLN | A | 171 | 58.292 | 54.940 | 73.332 | 1.00 | 57.67 | A | C |
| ATOM | 1284 | CG | GLN | A | 171 | 56.987 | 54.971 | 72.538 | 1.00 | 57.77 | A | C |
| ATOM | 1285 | CD | GLN | A | 171 | 57.229 | 55.256 | 71.063 | 1.00 | 58.21 | A | C |
| ATOM | 1286 | OE1 | GLN | A | 171 | 57.583 | 56.377 | 70.683 | 1.00 | 59.83 | A | O |
| ATOM | 1287 | NE2 | GLN | A | 171 | 57.061 | 54.236 | 70.227 | 1.00 | 52.86 | A | N |
| ATOM | 1288 | C | GLN | A | 171 | 60.701 | 54.212 | 73.254 | 1.00 | 61.63 | A | C |
| ATOM | 1289 | O | GLN | A | 171 | 61.523 | 54.645 | 72.445 | 1.00 | 61.70 | A | O |
| ATOM | 1290 | N | HIS | A | 172 | 60.989 | 53.972 | 74.529 | 1.00 | 62.71 | A | N |
| ATOM | 1291 | CA | HIS | A | 172 | 62.307 | 54.239 | 75.096 | 1.00 | 64.81 | A | C |
| ATOM | 1292 | CB | HIS | A | 172 | 62.281 | 53.962 | 76.600 | 1.00 | 65.82 | A | C |
| ATOM | 1293 | CG | HIS | A | 172 | 63.472 | 54.489 | 77.337 | 1.00 | 71.14 | A | C |
| ATOM | 1294 | CD2 | HIS | A | 172 | 64.381 | 53.866 | 78.123 | 1.00 | 72.85 | A | C |
| ATOM | 1295 | ND1 | HIS | A | 172 | 63.816 | 55.824 | 77.336 | 1.00 | 72.17 | A | N |
| ATOM | 1296 | CE1 | HIS | A | 172 | 64.884 | 56.000 | 78.092 | 1.00 | 73.63 | A | C |
| ATOM | 1297 | NE2 | HIS | A | 172 | 65.248 | 54.829 | 78.582 | 1.00 | 72.71 | A | N |
| ATOM | 1298 | C | HIS | A | 172 | 63.423 | 53.419 | 74.437 | 1.00 | 65.03 | A | C |
| ATOM | 1299 | O | HIS | A | 172 | 64.339 | 53.979 | 73.826 | 1.00 | 64.94 | A | O |
| ATOM | 1300 | N | THR | A | 173 | 63.344 | 52.095 | 74.549 | 1.00 | 64.58 | A | N |
| ATOM | 1301 | CA | THR | A | 173 | 64.368 | 51.233 | 73.972 | 1.00 | 63.85 | A | C |
| ATOM | 1302 | CB | THR | A | 173 | 64.035 | 49.745 | 74.180 | 1.00 | 65.03 | A | C |
| ATOM | 1303 | OG1 | THR | A | 173 | 62.811 | 49.424 | 73.511 | 1.00 | 64.08 | A | O |
| ATOM | 1304 | CG2 | THR | A | 173 | 63.897 | 49.441 | 75.659 | 1.00 | 68.21 | A | C |
| ATOM | 1305 | C | THR | A | 173 | 64.585 | 51.478 | 72.486 | 1.00 | 63.59 | A | C |
| ATOM | 1306 | O | THR | A | 173 | 65.722 | 51.530 | 72.018 | 1.00 | 63.05 | A | O |
| ATOM | 1307 | N | LEU | A | 174 | 63.494 | 51.637 | 71.747 | 1.00 | 63.37 | A | N |
| ATOM | 1308 | CA | LEU | A | 174 | 63.562 | 51.871 | 70.306 | 1.00 | 64.00 | A | C |
| ATOM | 1309 | CB | LEU | A | 174 | 62.154 | 51.888 | 69.706 | 1.00 | 62.06 | A | C |
| ATOM | 1310 | CG | LEU | A | 174 | 61.466 | 50.563 | 69.390 | 1.00 | 63.02 | A | C |
| ATOM | 1311 | CD1 | LEU | A | 174 | 60.043 | 50.840 | 68.914 | 1.00 | 62.85 | A | C |
| ATOM | 1312 | CD2 | LEU | A | 174 | 62.250 | 49.820 | 68.316 | 1.00 | 60.98 | A | C |
| ATOM | 1313 | C | LEU | A | 174 | 64.264 | 53.165 | 69.904 | 1.00 | 65.06 | A | C |
| ATOM | 1314 | O | LEU | A | 174 | 65.075 | 53.177 | 68.980 | 1.00 | 64.23 | A | O |
| ATOM | 1315 | N | ILE | A | 175 | 63.939 | 54.253 | 70.593 | 1.00 | 67.46 | A | N |
| ATOM | 1316 | CA | ILE | A | 175 | 64.504 | 55.562 | 70.280 | 1.00 | 70.98 | A | C |
| ATOM | 1317 | CB | ILE | A | 175 | 63.633 | 56.698 | 70.881 | 1.00 | 69.78 | A | C |
| ATOM | 1318 | CG2 | ILE | A | 175 | 64.273 | 58.048 | 70.597 | 1.00 | 69.76 | A | C |
| ATOM | 1319 | CG1 | ILE | A | 175 | 62.221 | 56.652 | 70.288 | 1.00 | 69.44 | A | C |
| ATOM | 1320 | CD1 | ILE | A | 175 | 61.251 | 57.647 | 70.908 | 1.00 | 64.41 | A | C |
| ATOM | 1321 | C | ILE | A | 175 | 65.947 | 55.792 | 70.732 | 1.00 | 73.52 | A | C |
| ATOM | 1322 | O | ILE | A | 175 | 66.777 | 56.271 | 69.956 | 1.00 | 73.60 | A | O |
| ATOM | 1323 | N | PHE | A | 176 | 66.247 | 55.439 | 71.976 | 1.00 | 74.94 | A | N |
| ATOM | 1324 | CA | PHE | A | 176 | 67.574 | 55.672 | 72.527 | 1.00 | 77.45 | A | C |
| ATOM | 1325 | CB | PHE | A | 176 | 67.419 | 56.034 | 74.001 | 1.00 | 75.23 | A | C |
| ATOM | 1326 | CG | PHE | A | 176 | 66.503 | 57.199 | 74.222 | 1.00 | 73.30 | A | C |
| ATOM | 1327 | CD1 | PHE | A | 176 | 66.796 | 58.441 | 73.664 | 1.00 | 72.66 | A | C |
| ATOM | 1328 | CD2 | PHE | A | 176 | 65.322 | 57.049 | 74.938 | 1.00 | 74.30 | A | C |
| ATOM | 1329 | CE1 | PHE | A | 176 | 65.924 | 59.518 | 73.813 | 1.00 | 73.58 | A | C |
| ATOM | 1330 | CE2 | PHE | A | 176 | 64.438 | 58.122 | 75.096 | 1.00 | 74.30 | A | C |
| ATOM | 1331 | CZ | PHE | A | 176 | 64.741 | 59.358 | 74.530 | 1.00 | 73.34 | A | C |
| ATOM | 1332 | C | PHE | A | 176 | 68.633 | 54.590 | 72.342 | 1.00 | 79.54 | A | C |
| ATOM | 1333 | O | PHE | A | 176 | 68.600 | 53.547 | 72.991 | 1.00 | 78.86 | A | O |
| ATOM | 1334 | N | ASP | A | 177 | 69.578 | 54.872 | 71.446 | 1.00 | 83.89 | A | N |
| ATOM | 1335 | CA | ASP | A | 177 | 70.688 | 53.972 | 71.135 | 1.00 | 86.60 | A | C |
| ATOM | 1336 | CB | ASP | A | 177 | 70.509 | 53.366 | 69.747 | 1.00 | 88.05 | A | C |
| ATOM | 1337 | CG | ASP | A | 177 | 69.241 | 52.571 | 69.625 | 1.00 | 90.19 | A | C |
| ATOM | 1338 | OD1 | ASP | A | 177 | 68.972 | 52.065 | 68.515 | 1.00 | 91.64 | A | O |
| ATOM | 1339 | OD2 | ASP | A | 177 | 68.514 | 52.451 | 70.635 | 1.00 | 90.98 | A | O |
| ATOM | 1340 | C | ASP | A | 177 | 72.004 | 54.742 | 71.163 | 1.00 | 86.75 | A | C |
| ATOM | 1341 | O | ASP | A | 177 | 72.403 | 55.328 | 70.154 | 1.00 | 86.99 | A | O |
| ATOM | 1342 | N | LYS | A | 186 | 61.479 | 60.217 | 60.608 | 1.00 | 78.19 | A | N |
| ATOM | 1343 | CA | LYS | A | 186 | 60.276 | 59.704 | 59.960 | 1.00 | 78.33 | A | C |
| ATOM | 1344 | CB | LYS | A | 186 | 60.658 | 58.749 | 58.827 | 1.00 | 82.05 | A | C |
| ATOM | 1345 | CG | LYS | A | 186 | 61.558 | 59.376 | 57.774 | 1.00 | 88.09 | A | C |
| ATOM | 1346 | CD | LYS | A | 186 | 60.828 | 60.437 | 56.960 | 1.00 | 92.73 | A | C |
| ATOM | 1347 | CE | LYS | A | 186 | 59.808 | 59.812 | 56.015 | 1.00 | 95.03 | A | C |
| ATOM | 1348 | NZ | LYS | A | 186 | 60.445 | 58.850 | 55.069 | 1.00 | 96.59 | A | N |
| ATOM | 1349 | C | LYS | A | 186 | 59.390 | 58.983 | 60.974 | 1.00 | 74.70 | A | C |
| ATOM | 1350 | O | LYS | A | 186 | 58.222 | 59.320 | 61.139 | 1.00 | 74.25 | A | O |
| ATOM | 1351 | N | HIS | A | 187 | 59.952 | 57.987 | 61.649 | 1.00 | 70.89 | A | N |
| ATOM | 1352 | CA | HIS | A | 187 | 59.214 | 57.234 | 62.656 | 1.00 | 68.12 | A | C |
| ATOM | 1353 | CB | HIS | A | 187 | 59.378 | 55.722 | 62.440 | 1.00 | 69.56 | A | C |
| ATOM | 1354 | CG | HIS | A | 187 | 58.487 | 55.145 | 61.381 | 1.00 | 70.72 | A | C |
| ATOM | 1355 | CD2 | HIS | A | 187 | 57.504 | 54.214 | 61.457 | 1.00 | 70.29 | A | C |
| ATOM | 1356 | ND1 | HIS | A | 187 | 58.593 | 55.479 | 60.047 | 1.00 | 70.87 | A | N |
| ATOM | 1357 | CE1 | HIS | A | 187 | 57.719 | 54.777 | 59.347 | 1.00 | 68.95 | A | C |
| ATOM | 1358 | NE2 | HIS | A | 187 | 57.046 | 54.001 | 60.178 | 1.00 | 69.42 | A | N |
| ATOM | 1359 | C | HIS | A | 187 | 59.715 | 57.588 | 64.051 | 1.00 | 65.71 | A | C |
| ATOM | 1360 | O | HIS | A | 187 | 60.918 | 57.636 | 64.297 | 1.00 | 64.99 | A | O |
| ATOM | 1361 | N | ILE | A | 188 | 58.790 | 57.839 | 64.966 | 1.00 | 60.86 | A | N |
| ATOM | 1362 | CA | ILE | A | 188 | 59.162 | 58.164 | 66.329 | 1.00 | 58.32 | A | C |
| ATOM | 1363 | CB | ILE | A | 188 | 58.280 | 59.293 | 66.895 | 1.00 | 58.11 | A | C |
| ATOM | 1364 | CG2 | ILE | A | 188 | 58.776 | 59.701 | 68.271 | 1.00 | 55.03 | A | C |
| ATOM | 1365 | CG1 | ILE | A | 188 | 58.294 | 60.492 | 65.942 | 1.00 | 59.74 | A | C |
| ATOM | 1366 | CD1 | ILE | A | 188 | 59.672 | 61.029 | 65.642 | 1.00 | 59.45 | A | C |
| ATOM | 1367 | C | ILE | A | 188 | 58.971 | 56.901 | 67.157 | 1.00 | 58.21 | A | C |
| ATOM | 1368 | O | ILE | A | 188 | 58.039 | 56.796 | 67.961 | 1.00 | 58.39 | A | O |
| ATOM | 1369 | N | GLY | A | 189 | 59.863 | 55.938 | 66.947 | 1.00 | 57.19 | A | N |
| ATOM | 1370 | CA | GLY | A | 189 | 59.777 | 54.679 | 67.665 | 1.00 | 53.36 | A | C |
| ATOM | 1371 | C | GLY | A | 189 | 58.768 | 53.772 | 66.982 | 1.00 | 53.10 | A | C |
| ATOM | 1372 | O | GLY | A | 189 | 58.945 | 53.407 | 65.813 | 1.00 | 48.42 | A | O |
| ATOM | 1373 | N | SER | A | 190 | 57.705 | 53.418 | 67.705 | 1.00 | 51.12 | A | N |
| ATOM | 1374 | CA | SER | A | 190 | 56.665 | 52.551 | 67.159 | 1.00 | 50.46 | A | C |
| ATOM | 1375 | CB | SER | A | 190 | 56.072 | 51.656 | 68.260 | 1.00 | 52.62 | A | C |
| ATOM | 1376 | OG | SER | A | 190 | 55.211 | 52.377 | 69.138 | 1.00 | 51.65 | A | O |
| ATOM | 1377 | C | SER | A | 190 | 55.550 | 53.382 | 66.528 | 1.00 | 49.31 | A | C |
| ATOM | 1378 | O | SER | A | 190 | 54.623 | 52.839 | 65.929 | 1.00 | 48.73 | A | O |
| ATOM | 1379 | N | ILE | A | 191 | 55.655 | 54.699 | 66.666 | 1.00 | 47.59 | A | N |
| ATOM | 1380 | CA | ILE | A | 191 | 54.660 | 55.622 | 66.130 | 1.00 | 48.02 | A | C |
| ATOM | 1381 | CB | ILE | A | 191 | 54.466 | 56.819 | 67.069 | 1.00 | 47.92 | A | C |
| ATOM | 1382 | CG2 | ILE | A | 191 | 53.543 | 57.839 | 66.431 | 1.00 | 43.98 | A | C |
| ATOM | 1383 | CG1 | ILE | A | 191 | 53.915 | 56.332 | 68.409 | 1.00 | 51.85 | A | C |
| ATOM | 1384 | CD1 | ILE | A | 191 | 53.944 | 57.378 | 69.490 | 1.00 | 51.07 | A | C |
| ATOM | 1385 | C | ILE | A | 191 | 55.034 | 56.167 | 64.760 | 1.00 | 47.96 | A | C |
| ATOM | 1386 | O | ILE | A | 191 | 56.183 | 56.539 | 64.531 | 1.00 | 46.22 | A | O |
| ATOM | 1387 | N | ASP | A | 192 | 54.056 | 56.199 | 63.853 | 1.00 | 46.65 | A | N |
| ATOM | 1388 | CA | ASP | A | 192 | 54.254 | 56.727 | 62.501 | 1.00 | 45.38 | A | C |
| ATOM | 1389 | CB | ASP | A | 192 | 53.838 | 55.717 | 61.435 | 1.00 | 44.26 | A | C |
| ATOM | 1390 | CG | ASP | A | 192 | 54.091 | 56.230 | 60.023 | 1.00 | 47.17 | A | C |
| ATOM | 1391 | CD1 | ASP | A | 192 | 54.278 | 57.458 | 59.864 | 1.00 | 43.77 | A | O |
| ATOM | 1392 | OD2 | ASP | A | 192 | 54.100 | 55.414 | 59.071 | 1.00 | 45.27 | A | O |
| ATOM | 1393 | C | ASP | A | 192 | 53.365 | 57.953 | 62.367 | 1.00 | 46.29 | A | C |
| ATOM | 1394 | O | ASP | A | 192 | 52.146 | 57.830 | 62.274 | 1.00 | 43.86 | A | O |
| ATOM | 1395 | N | PRO | A | 193 | 53.962 | 59.153 | 62.363 | 1.00 | 47.69 | A | N |
| ATOM | 1396 | CD | PRO | A | 193 | 55.403 | 59.448 | 62.467 | 1.00 | 48.61 | A | C |
| ATOM | 1397 | CA | PRO | A | 193 | 53.183 | 60.386 | 62.242 | 1.00 | 46.86 | A | C |
| ATOM | 1398 | CB | PRO | A | 193 | 54.229 | 61.476 | 62.487 | 1.00 | 46.08 | A | C |
| ATOM | 1399 | CG | PRO | A | 193 | 55.478 | 60.855 | 61.929 | 1.00 | 48.47 | A | C |
| ATOM | 1400 | C | PRO | A | 193 | 52.469 | 60.525 | 60.899 | 1.00 | 44.24 | A | C |
| ATOM | 1401 | O | PRO | A | 193 | 51.629 | 61.400 | 60.733 | 1.00 | 44.26 | A | O |
| ATOM | 1402 | N | ASN | A | 194 | 52.801 | 59.662 | 59.945 | 1.00 | 44.57 | A | N |
| ATOM | 1403 | CA | ASN | A | 194 | 52.155 | 59.701 | 58.634 | 1.00 | 45.05 | A | C |
| ATOM | 1404 | CB | ASN | A | 194 | 53.096 | 60.289 | 57.585 | 1.00 | 51.28 | A | C |
| ATOM | 1405 | CG | ASN | A | 194 | 52.934 | 61.780 | 57.439 | 1.00 | 60.03 | A | C |
| ATOM | 1406 | OD1 | ASN | A | 194 | 53.401 | 62.554 | 58.276 | 1.00 | 61.90 | A | O |
| ATOM | 1407 | ND2 | ASN | A | 194 | 52.249 | 62.196 | 56.375 | 1.00 | 64.04 | A | N |
| ATOM | 1408 | C | ASN | A | 194 | 51.698 | 58.322 | 58.168 | 1.00 | 42.39 | A | C |
| ATOM | 1409 | O | ASN | A | 194 | 51.852 | 57.963 | 57.001 | 1.00 | 43.58 | A | O |
| ATOM | 1410 | N | CYS | A | 195 | 51.130 | 57.555 | 59.083 | 1.00 | 41.72 | A | N |
| ATOM | 1411 | CA | CYS | A | 195 | 50.671 | 56.214 | 58.764 | 1.00 | 40.12 | A | C |
| ATOM | 1412 | CB | CYS | A | 195 | 50.146 | 55.536 | 60.025 | 1.00 | 41.51 | A | C |
| ATOM | 1413 | SG | CYS | A | 195 | 49.478 | 53.894 | 59.716 | 1.00 | 36.56 | A | S |
| ATOM | 1414 | C | CYS | A | 195 | 49.573 | 56.228 | 57.706 | 1.00 | 38.95 | A | C |
| ATOM | 1415 | O | CYS | A | 195 | 48.556 | 56.896 | 57.877 | 1.00 | 36.14 | A | O |
| ATOM | 1416 | N | ASN | A | 196 | 49.803 | 55.515 | 56.605 | 1.00 | 36.80 | A | N |
| ATOM | 1417 | CA | ASN | A | 196 | 48.813 | 55.406 | 55.543 | 1.00 | 40.69 | A | C |
| ATOM | 1418 | CB | ASN | A | 196 | 49.488 | 55.254 | 54.180 | 1.00 | 42.07 | A | C |
| ATOM | 1419 | CG | ASN | A | 196 | 48.487 | 54.954 | 53.079 | 1.00 | 44.50 | A | C |
| ATOM | 1420 | OD1 | ASN | A | 196 | 47.905 | 53.868 | 53.029 | 1.00 | 43.39 | A | O |
| ATOM | 1421 | ND2 | ASN | A | 196 | 48.267 | 55.923 | 52.202 | 1.00 | 47.97 | A | N |
| ATOM | 1422 | C | ASN | A | 196 | 47.977 | 54.156 | 55.849 | 1.00 | 39.50 | A | C |
| ATOM | 1423 | O | ASN | A | 196 | 48.434 | 53.025 | 55.670 | 1.00 | 35.27 | A | O |
| ATOM | 1424 | N | VAL | A | 197 | 46.750 | 54.361 | 56.309 | 1.00 | 39.08 | A | N |
| ATOM | 1425 | CA | VAL | A | 197 | 45.895 | 53.239 | 56.685 | 1.00 | 33.72 | A | C |
| ATOM | 1426 | CB | VAL | A | 197 | 44.520 | 53.762 | 57.158 | 1.00 | 34.17 | A | C |
| ATOM | 1427 | CG1 | VAL | A | 197 | 43.637 | 52.608 | 57.612 | 1.00 | 28.18 | A | C |
| ATOM | 1428 | CG2 | VAL | A | 197 | 44.728 | 54.740 | 58.310 | 1.00 | 28.70 | A | C |
| ATOM | 1429 | C | VAL | A | 197 | 45.706 | 52.131 | 55.647 | 1.00 | 35.02 | A | C |
| ATOM | 1430 | O | VAL | A | 197 | 45.856 | 50.944 | 55.968 | 1.00 | 33.96 | A | O |
| ATOM | 1431 | N | SER | A | 198 | 45.395 | 52.497 | 54.409 | 1.00 | 37.29 | A | N |
| ATOM | 1432 | CA | SER | A | 198 | 45.167 | 51.490 | 53.377 | 1.00 | 40.39 | A | C |
| ATOM | 1433 | CB | SER | A | 198 | 44.851 | 52.154 | 52.034 | 1.00 | 43.19 | A | C |
| ATOM | 1434 | OG | SER | A | 198 | 43.558 | 52.738 | 52.045 | 1.00 | 46.92 | A | O |
| ATOM | 1435 | C | SER | A | 198 | 46.340 | 50.546 | 53.197 | 1.00 | 41.97 | A | C |
| ATOM | 1436 | O | SER | A | 198 | 46.158 | 49.364 | 52.879 | 1.00 | 39.82 | A | O |
| ATOM | 1437 | N | GLU | A | 199 | 47.542 | 51.077 | 53.404 | 1.00 | 44.03 | A | N |
| ATOM | 1438 | CA | GLU | A | 199 | 48.766 | 50.306 | 53.241 | 1.00 | 42.51 | A | C |
| ATOM | 1439 | CB | GLU | A | 199 | 49.986 | 51.235 | 53.303 | 1.00 | 48.45 | A | C |
| ATOM | 1440 | CG | GLU | A | 199 | 51.303 | 50.566 | 52.917 | 1.00 | 58.71 | A | C |
| ATOM | 1441 | CD | GLU | A | 199 | 51.228 | 49.832 | 51.576 | 1.00 | 66.38 | A | C |
| ATOM | 1442 | OE1 | GLU | A | 199 | 50.492 | 50.300 | 50.668 | 1.00 | 67.34 | A | O |
| ATOM | 1443 | OE2 | GLU | A | 199 | 51.920 | 48.793 | 51.430 | 1.00 | 68.47 | A | O |
| ATOM | 1444 | C | GLU | A | 199 | 48.870 | 49.229 | 54.304 | 1.00 | 37.61 | A | C |
| ATOM | 1445 | O | GLU | A | 199 | 49.242 | 48.091 | 54.007 | 1.00 | 34.49 | A | O |
| ATOM | 1446 | N | VAL | A | 200 | 48.545 | 49.582 | 55.543 | 1.00 | 33.72 | A | N |
| ATOM | 1447 | CA | VAL | A | 200 | 48.605 | 48.605 | 56.617 | 1.00 | 35.06 | A | C |
| ATOM | 1448 | CB | VAL | A | 200 | 48.371 | 49.240 | 57.991 | 1.00 | 34.22 | A | C |
| ATOM | 1449 | CG1 | VAL | A | 200 | 48.478 | 48.183 | 59.058 | 1.00 | 35.70 | A | C |
| ATOM | 1450 | CG2 | VAL | A | 200 | 49.381 | 50.351 | 58.249 | 1.00 | 39.53 | A | C |
| ATOM | 1451 | C | VAL | A | 200 | 47.512 | 47.569 | 56.372 | 1.00 | 36.01 | A | C |
| ATOM | 1452 | O | VAL | A | 200 | 47.673 | 46.397 | 56.720 | 1.00 | 36.14 | A | O |
| ATOM | 1453 | N | VAL | A | 201 | 46.408 | 47.997 | 55.755 | 1.00 | 35.08 | A | N |
| ATOM | 1454 | CA | VAL | A | 201 | 45.306 | 47.076 | 55.477 | 1.00 | 33.31 | A | C |
| ATOM | 1455 | CB | VAL | A | 201 | 44.061 | 47.817 | 54.873 | 1.00 | 32.16 | A | C |
| ATOM | 1456 | CG1 | VAL | A | 201 | 43.008 | 46.798 | 54.415 | 1.00 | 26.21 | A | C |
| ATOM | 1457 | CG2 | VAL | A | 201 | 43.433 | 48.743 | 55.926 | 1.00 | 28.09 | A | C |
| ATOM | 1458 | C | VAL | A | 201 | 45.797 | 46.021 | 54.486 | 1.00 | 34.35 | A | C |
| ATOM | 1459 | O | VAL | A | 201 | 45.544 | 44.822 | 54.655 | 1.00 | 33.45 | A | O |
| ATOM | 1460 | N | LYS | A | 202 | 46.515 | 46.473 | 53.462 | 1.00 | 35.76 | A | N |
| ATOM | 1461 | CA | LYS | A | 202 | 47.041 | 45.571 | 52.440 | 1.00 | 37.80 | A | C |
| ATOM | 1462 | CB | LYS | A | 202 | 47.700 | 46.381 | 51.324 | 1.00 | 40.99 | A | C |
| ATOM | 1463 | CG | LYS | A | 202 | 46.699 | 47.281 | 50.594 | 1.00 | 46.57 | A | C |
| ATOM | 1464 | CD | LYS | A | 202 | 47.348 | 48.200 | 49.553 | 1.00 | 51.98 | A | C |
| ATOM | 1465 | CE | LYS | A | 202 | 46.348 | 49.265 | 49.076 | 1.00 | 57.00 | A | C |
| ATOM | 1466 | NZ | LYS | A | 202 | 46.931 | 50.276 | 48.144 | 1.00 | 59.89 | A | N |
| ATOM | 1467 | C | LYS | A | 202 | 48.016 | 44.565 | 53.038 | 1.00 | 37.24 | A | C |
| ATOM | 1468 | O | LYS | A | 202 | 47.932 | 43.364 | 52.758 | 1.00 | 38.80 | A | O |
| ATOM | 1469 | N | ASP | A | 203 | 48.929 | 45.049 | 53.875 | 1.00 | 37.14 | A | N |
| ATOM | 1470 | CA | ASP | A | 203 | 49.888 | 44.164 | 54.520 | 1.00 | 37.30 | A | C |
| ATOM | 1471 | CB | ASP | A | 203 | 50.811 | 44.921 | 55.488 | 1.00 | 39.55 | A | C |
| ATOM | 1472 | CG | ASP | A | 203 | 51.688 | 45.963 | 54.799 | 1.00 | 42.50 | A | C |
| ATOM | 1473 | OD1 | ASP | A | 203 | 52.019 | 45.824 | 53.599 | 1.00 | 46.47 | A | O |
| ATOM | 1474 | OD2 | ASP | A | 203 | 52.067 | 46.929 | 55.488 | 1.00 | 46.32 | A | O |
| ATOM | 1475 | C | ASP | A | 203 | 49.160 | 43.085 | 55.317 | 1.00 | 36.63 | A | C |
| ATOM | 1476 | O | ASP | A | 203 | 49.424 | 41.900 | 55.129 | 1.00 | 35.29 | A | O |
| ATOM | 1477 | N | ALA | A | 204 | 48.253 | 43.484 | 56.214 | 1.00 | 35.66 | A | N |
| ATOM | 1478 | CA | ALA | A | 204 | 47.536 | 42.495 | 57.027 | 1.00 | 32.67 | A | C |
| ATOM | 1479 | CB | ALA | A | 204 | 46.561 | 43.182 | 57.973 | 1.00 | 31.12 | A | C |
| ATOM | 1480 | C | ALA | A | 204 | 46.805 | 41.473 | 56.151 | 1.00 | 32.34 | A | C |
| ATOM | 1481 | O | ALA | A | 204 | 46.798 | 40.271 | 56.462 | 1.00 | 30.00 | A | O |
| ATOM | 1482 | N | TYR | A | 205 | 46.201 | 41.951 | 55.062 | 1.00 | 33.19 | A | N |
| ATOM | 1483 | CA | TYR | A | 205 | 45.492 | 41.075 | 54.131 | 1.00 | 36.12 | A | C |
| ATOM | 1484 | CB | TYR | A | 205 | 44.845 | 41.894 | 53.012 | 1.00 | 35.38 | A | C |
| ATOM | 1485 | CG | TYR | A | 205 | 44.343 | 41.051 | 51.861 | 1.00 | 40.78 | A | C |
| ATOM | 1486 | CD1 | TYR | A | 205 | 43.160 | 40.315 | 51.969 | 1.00 | 40.01 | A | C |
| ATOM | 1487 | CE1 | TYR | A | 205 | 42.718 | 39.491 | 50.922 | 1.00 | 42.07 | A | C |
| ATOM | 1488 | CD2 | TYR | A | 205 | 45.078 | 40.948 | 50.672 | 1.00 | 42.93 | A | C |
| ATOM | 1489 | CE2 | TYR | A | 205 | 44.650 | 40.121 | 49.623 | 1.00 | 42.73 | A | C |
| ATOM | 1490 | CZ | TYR | A | 205 | 43.471 | 39.398 | 49.759 | 1.00 | 44.04 | A | C |
| ATOM | 1491 | OH | TYR | A | 205 | 43.061 | 38.571 | 48.746 | 1.00 | 39.93 | A | O |
| ATOM | 1492 | C | TYR | A | 205 | 46.478 | 40.077 | 53.507 | 1.00 | 38.37 | A | C |
| ATOM | 1493 | O | TYR | A | 205 | 46.224 | 38.868 | 53.490 | 1.00 | 37.00 | A | O |
| ATOM | 1494 | N | ASP | A | 206 | 47.601 | 40.584 | 52.997 | 1.00 | 37.76 | A | N |
| ATOM | 1495 | CA | ASP | A | 206 | 48.605 | 39.716 | 52.364 | 1.00 | 41.15 | A | C |
| ATOM | 1496 | CB | ASP | A | 206 | 49.847 | 40.520 | 51.946 | 1.00 | 42.52 | A | C |
| ATOM | 1497 | CG | ASP | A | 206 | 49.561 | 41.500 | 50.820 | 1.00 | 45.51 | A | C |
| ATOM | 1498 | OD1 | ASP | A | 206 | 48.682 | 41.216 | 49.976 | 1.00 | 51.20 | A | O |
| ATOM | 1499 | OD2 | ASP | A | 206 | 50.228 | 42.551 | 50.766 | 1.00 | 48.44 | A | O |
| ATOM | 1500 | C | ASP | A | 206 | 49.028 | 38.544 | 53.246 | 1.00 | 38.53 | A | C |
| ATOM | 1501 | O | ASP | A | 206 | 49.063 | 37.402 | 52.791 | 1.00 | 39.84 | A | O |
| ATOM | 1502 | N | MET | A | 207 | 49.344 | 38.821 | 54.504 | 1.00 | 38.04 | A | N |
| ATOM | 1503 | CA | MET | A | 207 | 49.748 | 37.762 | 55.426 | 1.00 | 40.49 | A | C |
| ATOM | 1504 | CB | MET | A | 207 | 50.103 | 38.332 | 56.800 | 1.00 | 41.12 | A | C |
| ATOM | 1505 | CG | MET | A | 207 | 51.576 | 38.299 | 57.162 | 1.00 | 51.28 | A | C |
| ATOM | 1506 | SD | MET | A | 207 | 52.401 | 36.714 | 56.868 | 1.00 | 61.15 | A | S |
| ATOM | 1507 | CE | MET | A | 207 | 51.614 | 35.629 | 58.061 | 1.00 | 55.29 | A | C |
| ATOM | 1508 | C | MET | A | 207 | 48.623 | 36.753 | 55.613 | 1.00 | 42.56 | A | C |
| ATOM | 1509 | O | MET | A | 207 | 48.851 | 35.542 | 55.572 | 1.00 | 42.41 | A | O |
| ATOM | 1510 | N | ALA | A | 208 | 47.411 | 37.257 | 55.850 | 1.00 | 40.73 | A | N |
| ATOM | 1511 | CA | ALA | A | 208 | 46.255 | 36.388 | 56.058 | 1.00 | 41.89 | A | C |
| ATOM | 1512 | CB | ALA | A | 208 | 45.017 | 37.223 | 56.385 | 1.00 | 41.31 | A | C |
| ATOM | 1513 | C | ALA | A | 208 | 45.998 | 35.520 | 54.828 | 1.00 | 38.83 | A | C |
| ATOM | 1514 | O | ALA | A | 208 | 45.720 | 34.326 | 54.953 | 1.00 | 36.61 | A | O |
| ATOM | 1515 | N | LYS | A | 209 | 46.087 | 36.127 | 53.647 | 1.00 | 39.24 | A | N |
| ATOM | 1516 | CA | LYS | A | 209 | 45.888 | 35.398 | 52.397 | 1.00 | 44.63 | A | C |
| ATOM | 1517 | CB | LYS | A | 209 | 46.055 | 36.333 | 51.194 | 1.00 | 41.70 | A | C |
| ATOM | 1518 | CG | LYS | A | 209 | 46.100 | 35.606 | 49.847 | 1.00 | 47.35 | A | C |
| ATOM | 1519 | CD | LYS | A | 209 | 45.914 | 36.577 | 48.677 | 1.00 | 47.99 | A | C |
| ATOM | 1520 | CE | LYS | A | 209 | 46.352 | 35.963 | 47.346 | 1.00 | 54.42 | A | C |
| ATOM | 1521 | NZ | LYS | A | 209 | 45.768 | 34.610 | 47.073 | 1.00 | 58.37 | A | N |
| ATOM | 1522 | C | LYS | A | 209 | 46.907 | 34.260 | 52.307 | 1.00 | 47.68 | A | C |
| ATOM | 1523 | O | LYS | A | 209 | 46.564 | 33.112 | 51.988 | 1.00 | 45.26 | A | O |
| ATOM | 1524 | N | LEU | A | 210 | 48.159 | 34.587 | 52.609 | 1.00 | 47.54 | A | N |
| ATOM | 1525 | CA | LEU | A | 210 | 49.224 | 33.600 | 52.566 | 1.00 | 51.28 | A | C |
| ATOM | 1526 | CB | LEU | A | 210 | 50.525 | 34.184 | 53.118 | 1.00 | 55.02 | A | C |
| ATOM | 1527 | CG | LEU | A | 210 | 51.785 | 33.446 | 52.658 | 1.00 | 57.14 | A | C |
| ATOM | 1528 | CD1 | LEU | A | 210 | 52.130 | 33.955 | 51.256 | 1.00 | 59.14 | A | C |
| ATOM | 1529 | CD2 | LEU | A | 210 | 52.954 | 33.679 | 53.627 | 1.00 | 56.45 | A | C |
| ATOM | 1530 | C | LEU | A | 210 | 48.851 | 32.377 | 53.384 | 1.00 | 50.87 | A | C |
| ATOM | 1531 | O | LEU | A | 210 | 48.906 | 31.249 | 52.887 | 1.00 | 51.74 | A | O |
| ATOM | 1532 | N | LEU | A | 211 | 48.464 | 32.601 | 54.637 | 1.00 | 48.57 | A | N |
| ATOM | 1533 | CA | LEU | A | 211 | 48.100 | 31.509 | 55.527 | 1.00 | 48.47 | A | C |
| ATOM | 1534 | CB | LEU | A | 211 | 47.898 | 32.027 | 56.946 | 1.00 | 49.29 | A | C |
| ATOM | 1535 | CG | LEU | A | 211 | 49.134 | 32.577 | 57.653 | 1.00 | 50.33 | A | C |
| ATOM | 1536 | CD1 | LEU | A | 211 | 48.723 | 33.168 | 58.991 | 1.00 | 48.14 | A | C |
| ATOM | 1537 | CD2 | LEU | A | 211 | 50.154 | 31.458 | 57.837 | 1.00 | 47.52 | A | C |
| ATOM | 1538 | C | LEU | A | 211 | 46.834 | 30.815 | 55.070 | 1.00 | 52.37 | A | C |
| ATOM | 1539 | O | LEU | A | 211 | 46.656 | 29.607 | 55.275 | 1.00 | 51.16 | A | O |
| ATOM | 1540 | N | CYS | A | 212 | 45.947 | 31.587 | 54.453 | 1.00 | 53.09 | A | N |
| ATOM | 1541 | CA | CYS | A | 212 | 44.689 | 31.042 | 53.987 | 1.00 | 52.43 | A | C |
| ATOM | 1542 | CB | CYS | A | 212 | 43.762 | 32.161 | 53.509 | 1.00 | 51.72 | A | C |
| ATOM | 1543 | SG | CYS | A | 212 | 42.071 | 31.586 | 53.297 | 1.00 | 53.90 | A | S |
| ATOM | 1544 | C | CYS | A | 212 | 44.934 | 30.050 | 52.855 | 1.00 | 52.76 | A | C |
| ATOM | 1545 | O | CYS | A | 212 | 44.353 | 28.958 | 52.835 | 1.00 | 46.65 | A | O |
| ATOM | 1546 | N | ASP | A | 213 | 45.797 | 30.434 | 51.918 | 1.00 | 52.96 | A | N |
| ATOM | 1547 | CA | ASP | A | 213 | 46.116 | 29.570 | 50.792 | 1.00 | 54.09 | A | C |
| ATOM | 1548 | CB | ASP | A | 213 | 46.958 | 30.330 | 49.763 | 1.00 | 47.05 | A | C |
| ATOM | 1549 | CG | ASP | A | 213 | 46.129 | 31.322 | 48.952 | 1.00 | 50.24 | A | C |
| ATOM | 1550 | OD1 | ASP | A | 213 | 44.921 | 31.055 | 48.750 | 1.00 | 50.56 | A | O |
| ATOM | 1551 | OD2 | ASP | A | 213 | 46.676 | 32.354 | 48.497 | 1.00 | 44.28 | A | O |
| ATOM | 1552 | C | ASP | A | 213 | 46.830 | 28.293 | 51.246 | 1.00 | 55.99 | A | C |
| ATOM | 1553 | O | ASP | A | 213 | 46.561 | 27.213 | 50.727 | 1.00 | 57.19 | A | O |
| ATOM | 1554 | N | LYS | A | 214 | 47.723 | 28.412 | 52.225 | 1.00 | 59.65 | A | N |
| ATOM | 1555 | CA | LYS | A | 214 | 48.452 | 27.255 | 52.740 | 1.00 | 63.73 | A | C |
| ATOM | 1556 | CB | LYS | A | 214 | 49.449 | 27.681 | 53.824 | 1.00 | 64.78 | A | C |
| ATOM | 1557 | CG | LYS | A | 214 | 50.778 | 28.198 | 53.300 | 1.00 | 69.40 | A | C |
| ATOM | 1558 | CD | LYS | A | 214 | 51.612 | 27.078 | 52.695 | 1.00 | 73.26 | A | C |
| ATOM | 1559 | CE | LYS | A | 214 | 52.897 | 27.612 | 52.076 | 1.00 | 73.50 | A | C |
| ATOM | 1560 | NZ | LYS | A | 214 | 53.727 | 26.523 | 51.496 | 1.00 | 75.88 | A | N |
| ATOM | 1561 | C | LYS | A | 214 | 47.524 | 26.197 | 53.326 | 1.00 | 65.50 | A | C |
| ATOM | 1562 | O | LYS | A | 214 | 47.663 | 25.010 | 53.044 | 1.00 | 66.80 | A | O |
| ATOM | 1563 | N | TYR | A | 215 | 46.571 | 26.630 | 54.139 | 1.00 | 65.79 | A | N |
| ATOM | 1564 | CA | TYR | A | 215 | 45.656 | 25.706 | 54.783 | 1.00 | 66.14 | A | C |
| ATOM | 1565 | CB | TYR | A | 215 | 45.212 | 26.312 | 56.119 | 1.00 | 71.64 | A | C |
| ATOM | 1566 | CG | TYR | A | 215 | 44.354 | 25.421 | 56.998 | 1.00 | 77.75 | A | C |
| ATOM | 1567 | CD1 | TYR | A | 215 | 42.986 | 25.271 | 56.759 | 1.00 | 79.14 | A | C |
| ATOM | 1568 | CE1 | TYR | A | 215 | 42.196 | 24.464 | 57.578 | 1.00 | 80.98 | A | C |
| ATOM | 1569 | CD2 | TYR | A | 215 | 44.911 | 24.738 | 58.082 | 1.00 | 80.05 | A | C |
| ATOM | 1570 | CE2 | TYR | A | 215 | 44.129 | 23.930 | 58.906 | 1.00 | 81.17 | A | C |
| ATOM | 1571 | CZ | TYR | A | 215 | 42.775 | 23.796 | 58.649 | 1.00 | 81.12 | A | C |
| ATOM | 1572 | OH | TYR | A | 215 | 42.007 | 22.987 | 59.456 | 1.00 | 79.10 | A | O |
| ATOM | 1573 | C | TYR | A | 215 | 44.444 | 25.318 | 53.933 | 1.00 | 65.07 | A | C |
| ATOM | 1574 | O | TYR | A | 215 | 44.074 | 24.144 | 53.885 | 1.00 | 63.28 | A | O |
| ATOM | 1575 | N | TYR | A | 216 | 43.840 | 26.290 | 53.251 | 1.00 | 64.46 | A | N |
| ATOM | 1576 | CA | TYR | A | 216 | 42.648 | 26.026 | 52.439 | 1.00 | 63.04 | A | C |
| ATOM | 1577 | CB | TYR | A | 216 | 41.573 | 27.101 | 52.681 | 1.00 | 60.40 | A | C |
| ATOM | 1578 | CG | TYR | A | 216 | 41.053 | 27.158 | 54.100 | 1.00 | 58.26 | A | C |
| ATOM | 1579 | CD1 | TYR | A | 216 | 41.547 | 28.092 | 55.007 | 1.00 | 54.88 | A | C |
| ATOM | 1580 | CE1 | TYR | A | 216 | 41.100 | 28.118 | 56.333 | 1.00 | 56.17 | A | C |
| ATOM | 1581 | CD2 | TYR | A | 216 | 40.093 | 26.246 | 54.546 | 1.00 | 58.25 | A | C |
| ATOM | 1582 | CE2 | TYR | A | 216 | 39.639 | 26.260 | 55.866 | 1.00 | 57.37 | A | C |
| ATOM | 1583 | CZ | TYR | A | 216 | 40.147 | 27.195 | 56.759 | 1.00 | 58.51 | A | C |
| ATOM | 1584 | OH | TYR | A | 216 | 39.712 | 27.190 | 58.073 | 1.00 | 53.47 | A | O |
| ATOM | 1585 | C | TYR | A | 216 | 42.861 | 25.903 | 50.941 | 1.00 | 63.74 | A | C |
| ATOM | 1586 | O | TYR | A | 216 | 41.968 | 25.450 | 50.229 | 1.00 | 63.77 | A | O |
| ATOM | 1587 | N | MET | A | 217 | 44.023 | 26.314 | 50.450 | 1.00 | 65.17 | A | N |
| ATOM | 1588 | CA | MET | A | 217 | 44.295 | 26.233 | 49.018 | 1.00 | 67.58 | A | C |
| ATOM | 1589 | CB | MET | A | 217 | 44.332 | 24.764 | 48.583 | 1.00 | 74.17 | A | C |
| ATOM | 1590 | CG | MET | A | 217 | 45.419 | 23.953 | 49.291 | 1.00 | 81.46 | A | C |
| ATOM | 1591 | SD | MET | A | 217 | 45.244 | 22.164 | 49.092 | 1.00 | 91.60 | A | S |
| ATOM | 1592 | CE | MET | A | 217 | 44.290 | 21.721 | 50.591 | 1.00 | 89.56 | A | C |
| ATOM | 1593 | C | MET | A | 217 | 43.223 | 27.009 | 48.242 | 1.00 | 65.88 | A | C |
| ATOM | 1594 | O | MET | A | 217 | 42.817 | 26.635 | 47.138 | 1.00 | 64.88 | A | O |
| ATOM | 1595 | N | ALA | A | 218 | 42.770 | 28.099 | 48.853 | 1.00 | 62.00 | A | N |
| ATOM | 1596 | CA | ALA | A | 218 | 41.763 | 28.985 | 48.276 | 1.00 | 55.73 | A | C |
| ATOM | 1597 | CB | ALA | A | 218 | 40.424 | 28.274 | 48.177 | 1.00 | 56.03 | A | C |
| ATOM | 1598 | C | ALA | A | 218 | 41.664 | 30.190 | 49.210 | 1.00 | 51.27 | A | C |
| ATOM | 1599 | O | ALA | A | 218 | 41.931 | 30.071 | 50.403 | 1.00 | 47.76 | A | O |
| ATOM | 1600 | N | SER | A | 219 | 41.313 | 31.350 | 48.669 | 1.00 | 47.46 | A | N |
| ATOM | 1601 | CA | SER | A | 219 | 41.199 | 32.546 | 49.489 | 1.00 | 46.43 | A | C |
| ATOM | 1602 | CB | SER | A | 219 | 42.587 | 33.043 | 49.915 | 1.00 | 47.45 | A | C |
| ATOM | 1603 | OG | SER | A | 219 | 43.298 | 33.581 | 48.808 | 1.00 | 48.64 | A | O |
| ATOM | 1604 | C | SER | A | 219 | 40.485 | 33.641 | 48.715 | 1.00 | 44.64 | A | C |
| ATOM | 1605 | O | SER | A | 219 | 40.688 | 33.796 | 47.515 | 1.00 | 40.30 | A | O |
| ATOM | 1606 | N | PRO | A | 220 | 39.642 | 34.430 | 49.403 | 1.00 | 43.59 | A | N |
| ATOM | 1607 | CD | PRO | A | 220 | 39.325 | 34.388 | 50.843 | 1.00 | 42.49 | A | C |
| ATOM | 1608 | CA | PRO | A | 220 | 38.912 | 35.510 | 48.739 | 1.00 | 40.80 | A | C |
| ATOM | 1609 | CB | PRO | A | 220 | 37.857 | 35.886 | 49.774 | 1.00 | 43.55 | A | C |
| ATOM | 1610 | CG | PRO | A | 220 | 38.610 | 35.711 | 51.055 | 1.00 | 43.33 | A | C |
| ATOM | 1611 | C | PRO | A | 220 | 39.831 | 36.676 | 48.412 | 1.00 | 37.83 | A | C |
| ATOM | 1612 | O | PRO | A | 220 | 40.878 | 36.829 | 49.025 | 1.00 | 36.04 | A | O |
| ATOM | 1613 | N | ASP | A | 221 | 39.436 | 37.502 | 47.456 | 1.00 | 36.08 | A | N |
| ATOM | 1614 | CA | ASP | A | 221 | 40.236 | 38.661 | 47.095 | 1.00 | 39.14 | A | C |
| ATOM | 1615 | CB | ASP | A | 221 | 39.989 | 39.031 | 45.633 | 1.00 | 42.14 | A | C |
| ATOM | 1616 | CG | ASP | A | 221 | 40.458 | 37.938 | 44.676 | 1.00 | 47.81 | A | C |
| ATOM | 1617 | OD1 | ASP | A | 221 | 41.566 | 37.386 | 44.900 | 1.00 | 44.73 | A | O |
| ATOM | 1618 | OD2 | ASP | A | 221 | 39.722 | 37.633 | 43.712 | 1.00 | 45.48 | A | O |
| ATOM | 1619 | C | ASP | A | 221 | 39.901 | 39.841 | 48.008 | 1.00 | 41.36 | A | C |
| ATOM | 1620 | O | ASP | A | 221 | 39.072 | 39.714 | 48.908 | 1.00 | 42.47 | A | O |
| ATOM | 1621 | N | LEU | A | 222 | 40.539 | 40.980 | 47.771 | 1.00 | 39.64 | A | N |
| ATOM | 1622 | CA | LEU | A | 222 | 40.317 | 42.175 | 48.576 | 1.00 | 40.75 | A | C |
| ATOM | 1623 | CB | LEU | A | 222 | 41.586 | 42.543 | 49.358 | 1.00 | 38.78 | A | C |
| ATOM | 1624 | CG | LEU | A | 222 | 41.673 | 43.958 | 49.961 | 1.00 | 36.09 | A | C |
| ATOM | 1625 | CD1 | LEU | A | 222 | 40.752 | 44.113 | 51.167 | 1.00 | 28.16 | A | C |
| ATOM | 1626 | CD2 | LEU | A | 222 | 43.110 | 44.221 | 50.384 | 1.00 | 36.59 | A | C |
| ATOM | 1627 | C | LEU | A | 222 | 39.938 | 43.372 | 47.741 | 1.00 | 39.51 | A | C |
| ATOM | 1628 | O | LEU | A | 222 | 40.465 | 43.567 | 46.652 | 1.00 | 42.24 | A | O |
| ATOM | 1629 | N | GLU | A | 223 | 39.019 | 44.172 | 48.263 | 1.00 | 35.59 | A | N |
| ATOM | 1630 | CA | GLU | A | 223 | 38.611 | 45.407 | 47.610 | 1.00 | 37.56 | A | C |
| ATOM | 1631 | CB | GLU | A | 223 | 37.169 | 45.330 | 47.113 | 1.00 | 37.08 | A | C |
| ATOM | 1632 | CG | GLU | A | 223 | 36.983 | 44.572 | 45.829 | 1.00 | 41.59 | A | C |
| ATOM | 1633 | CD | GLU | A | 223 | 35.512 | 44.471 | 45.435 | 1.00 | 46.61 | A | C |
| ATOM | 1634 | OE1 | GLU | A | 223 | 34.770 | 45.471 | 45.612 | 1.00 | 44.24 | A | O |
| ATOM | 1635 | OE2 | GLU | A | 223 | 35.111 | 43.392 | 44.941 | 1.00 | 43.76 | A | O |
| ATOM | 1636 | C | GLU | A | 223 | 38.707 | 46.476 | 48.696 | 1.00 | 37.79 | A | C |
| ATOM | 1637 | O | GLU | A | 223 | 38.215 | 46.278 | 49.816 | 1.00 | 37.16 | A | O |
| ATOM | 1638 | N | ILE | A | 224 | 39.343 | 47.596 | 48.379 | 1.00 | 34.37 | A | N |
| ATOM | 1639 | CA | ILE | A | 224 | 39.465 | 48.670 | 49.348 | 1.00 | 37.44 | A | C |
| ATOM | 1640 | CB | ILE | A | 224 | 40.940 | 48.954 | 49.733 | 1.00 | 39.11 | A | C |
| ATOM | 1641 | CG2 | ILE | A | 224 | 41.012 | 50.180 | 50.647 | 1.00 | 36.50 | A | C |
| ATOM | 1642 | CG1 | ILE | A | 224 | 41.554 | 47.725 | 50.410 | 1.00 | 39.28 | A | C |
| ATOM | 1643 | CD1 | ILE | A | 224 | 42.998 | 47.928 | 50.849 | 1.00 | 45.24 | A | C |
| ATOM | 1644 | C | ILE | A | 224 | 38.885 | 49.929 | 48.760 | 1.00 | 37.01 | A | C |
| ATOM | 1645 | O | ILE | A | 224 | 39.048 | 50.197 | 47.583 | 1.00 | 39.37 | A | O |
| ATOM | 1646 | N | GLN | A | 225 | 38.215 | 50.706 | 49.600 | 1.00 | 39.04 | A | N |
| ATOM | 1647 | CA | GLN | A | 225 | 37.609 | 51.957 | 49.187 | 1.00 | 38.38 | A | C |
| ATOM | 1648 | CB | GLN | A | 225 | 36.120 | 51.732 | 48.900 | 1.00 | 42.93 | A | C |
| ATOM | 1649 | CG | GLN | A | 225 | 35.364 | 52.968 | 48.447 | 1.00 | 52.99 | A | C |
| ATOM | 1650 | CD | GLN | A | 225 | 33.851 | 52.757 | 48.461 | 1.00 | 59.86 | A | C |
| ATOM | 1651 | OE1 | GLN | A | 225 | 33.347 | 51.725 | 48.002 | 1.00 | 64.66 | A | O |
| ATOM | 1652 | NE2 | GLN | A | 225 | 33.122 | 53.735 | 48.984 | 1.00 | 59.24 | A | N |
| ATOM | 1653 | C | GLN | A | 225 | 37.750 | 52.969 | 50.316 | 1.00 | 37.73 | A | C |
| ATOM | 1654 | O | GLN | A | 225 | 37.447 | 52.658 | 51.465 | 1.00 | 38.26 | A | O |
| ATOM | 1655 | N | GLU | A | 226 | 38.230 | 54.171 | 50.023 | 1.00 | 35.03 | A | N |
| ATOM | 1656 | CA | GLU | A | 226 | 38.297 | 55.144 | 51.087 | 1.00 | 39.21 | A | C |
| ATOM | 1657 | CB | GLU | A | 226 | 39.747 | 55.542 | 51.436 | 1.00 | 45.12 | A | C |
| ATOM | 1658 | CG | GLU | A | 226 | 40.735 | 55.831 | 50.344 | 1.00 | 46.44 | A | C |
| ATOM | 1659 | CD | GLU | A | 226 | 42.146 | 56.038 | 50.923 | 1.00 | 52.28 | A | C |
| ATOM | 1660 | OE1 | GLU | A | 226 | 42.779 | 55.047 | 51.345 | 1.00 | 51.72 | A | O |
| ATOM | 1661 | OE2 | GLU | A | 226 | 42.618 | 57.193 | 50.978 | 1.00 | 52.42 | A | O |
| ATOM | 1662 | C | GLU | A | 226 | 37.414 | 56.356 | 50.834 | 1.00 | 38.24 | A | C |
| ATOM | 1663 | O | GLU | A | 226 | 37.072 | 56.666 | 49.697 | 1.00 | 37.95 | A | O |
| ATOM | 1664 | N | ILE | A | 227 | 37.001 | 56.995 | 51.924 | 1.00 | 38.61 | A | N |
| ATOM | 1665 | CA | ILE | A | 227 | 36.130 | 58.161 | 51.879 | 1.00 | 36.59 | A | C |
| ATOM | 1666 | CB | ILE | A | 227 | 34.739 | 57.863 | 52.500 | 1.00 | 37.67 | A | C |
| ATOM | 1667 | CG2 | ILE | A | 227 | 33.803 | 59.028 | 52.256 | 1.00 | 37.22 | A | C |
| ATOM | 1668 | CG1 | ILE | A | 227 | 34.137 | 56.583 | 51.916 | 1.00 | 38.56 | A | C |
| ATOM | 1669 | CD1 | ILE | A | 227 | 33.653 | 56.706 | 50.484 | 1.00 | 43.68 | A | C |
| ATOM | 1670 | C | ILE | A | 227 | 36.776 | 59.247 | 52.720 | 1.00 | 39.64 | A | C |
| ATOM | 1671 | O | ILE | A | 227 | 36.691 | 59.227 | 53.948 | 1.00 | 39.62 | A | O |
| ATOM | 1672 | N | ASN | A | 228 | 37.438 | 60.185 | 52.061 | 1.00 | 40.24 | A | N |
| ATOM | 1673 | CA | ASN | A | 228 | 38.080 | 61.284 | 52.762 | 1.00 | 42.50 | A | C |
| ATOM | 1674 | CB | ASN | A | 228 | 39.369 | 61.691 | 52.045 | 1.00 | 37.65 | A | C |
| ATOM | 1675 | CG | ASN | A | 228 | 40.221 | 62.627 | 52.871 | 1.00 | 38.75 | A | C |
| ATOM | 1676 | OD1 | ASN | A | 228 | 39.705 | 63.475 | 53.601 | 1.00 | 39.51 | A | O |
| ATOM | 1677 | ND2 | ASN | A | 228 | 41.538 | 62.492 | 52.747 | 1.00 | 39.68 | A | N |
| ATOM | 1678 | C | ASN | A | 228 | 37.067 | 62.420 | 52.715 | 1.00 | 44.48 | A | C |
| ATOM | 1679 | O | ASN | A | 228 | 37.108 | 63.266 | 51.819 | 1.00 | 47.19 | A | O |
| ATOM | 1680 | N | ALA | A | 229 | 36.157 | 62.424 | 53.684 | 1.00 | 42.68 | A | N |
| ATOM | 1681 | CA | ALA | A | 229 | 35.092 | 63.413 | 53.762 | 1.00 | 46.11 | A | C |
| ATOM | 1682 | CB | ALA | A | 229 | 34.419 | 63.342 | 55.134 | 1.00 | 42.39 | A | C |
| ATOM | 1683 | C | ALA | A | 229 | 35.479 | 64.857 | 53.448 | 1.00 | 48.84 | A | C |
| ATOM | 1684 | O | ALA | A | 229 | 34.858 | 65.487 | 52.603 | 1.00 | 50.04 | A | O |
| ATOM | 1685 | N | ALA | A | 230 | 36.499 | 65.380 | 54.119 | 1.00 | 52.17 | A | N |
| ATOM | 1686 | CA | ALA | A | 230 | 36.911 | 66.765 | 53.909 | 1.00 | 53.82 | A | C |
| ATOM | 1687 | CB | ALA | A | 230 | 37.572 | 67.299 | 55.175 | 1.00 | 51.90 | A | C |
| ATOM | 1688 | C | ALA | A | 230 | 37.818 | 67.025 | 52.701 | 1.00 | 56.06 | A | C |
| ATOM | 1689 | O | ALA | A | 230 | 37.899 | 68.162 | 52.238 | 1.00 | 55.13 | A | O |
| ATOM | 1690 | N | ASN | A | 231 | 38.496 | 65.989 | 52.199 | 1.00 | 58.18 | A | N |
| ATOM | 1691 | CA | ASN | A | 231 | 39.396 | 66.133 | 51.048 | 1.00 | 58.33 | A | C |
| ATOM | 1692 | CB | ASN | A | 231 | 40.837 | 66.323 | 51.516 | 1.00 | 60.37 | A | C |
| ATOM | 1693 | CG | ASN | A | 231 | 41.035 | 67.617 | 52.278 | 1.00 | 64.12 | A | C |
| ATOM | 1694 | OD1 | ASN | A | 231 | 40.604 | 67.749 | 53.425 | 1.00 | 63.73 | A | O |
| ATOM | 1695 | ND2 | ASN | A | 231 | 41.681 | 68.587 | 51.638 | 1.00 | 64.62 | A | N |
| ATOM | 1696 | C | ASN | A | 231 | 39.324 | 64.945 | 50.090 | 1.00 | 59.54 | A | C |
| ATOM | 1697 | O | ASN | A | 231 | 40.250 | 64.140 | 49.991 | 1.00 | 59.08 | A | O |
| ATOM | 1698 | N | SER | A | 232 | 38.210 | 64.888 | 49.370 | 1.00 | 62.87 | A | N |
| ATOM | 1699 | CA | SER | A | 232 | 37.862 | 63.854 | 48.394 | 1.00 | 66.60 | A | C |
| ATOM | 1700 | CB | SER | A | 232 | 37.117 | 64.509 | 47.222 | 1.00 | 67.75 | A | C |
| ATOM | 1701 | OG | SER | A | 232 | 36.490 | 63.537 | 46.403 | 1.00 | 67.79 | A | O |
| ATOM | 1702 | C | SER | A | 232 | 38.891 | 62.868 | 47.827 | 1.00 | 69.07 | A | C |
| ATOM | 1703 | O | SER | A | 232 | 38.696 | 61.658 | 47.913 | 1.00 | 69.56 | A | O |
| ATOM | 1704 | N | LYS | A | 233 | 39.973 | 63.339 | 47.226 | 1.00 | 72.91 | A | N |
| ATOM | 1705 | CA | LYS | A | 233 | 40.901 | 62.366 | 46.660 | 1.00 | 77.41 | A | C |
| ATOM | 1706 | CB | LYS | A | 233 | 41.172 | 62.693 | 45.188 | 1.00 | 83.92 | A | C |
| ATOM | 1707 | CG | LYS | A | 233 | 39.957 | 62.487 | 44.280 | 1.00 | 91.09 | A | C |
| ATOM | 1708 | CD | LYS | A | 233 | 39.308 | 61.116 | 44.512 | 1.00 | 95.31 | A | C |
| ATOM | 1709 | CE | LYS | A | 233 | 38.105 | 60.880 | 43.602 | 1.00 | 96.61 | A | C |
| ATOM | 1710 | NZ | LYS | A | 233 | 38.497 | 60.714 | 42.173 | 1.00 | 99.24 | A | N |
| ATOM | 1711 | C | LYS | A | 233 | 42.212 | 62.154 | 47.394 | 1.00 | 75.82 | A | C |
| ATOM | 1712 | O | LYS | A | 233 | 42.986 | 61.262 | 47.039 | 1.00 | 76.57 | A | O |
| ATOM | 1713 | N | GLN | A | 234 | 42.459 | 62.955 | 48.422 | 1.00 | 72.92 | A | N |
| ATOM | 1714 | CA | GLN | A | 234 | 43.690 | 62.822 | 49.186 | 1.00 | 70.10 | A | C |
| ATOM | 1715 | CB | GLN | A | 234 | 43.840 | 64.003 | 50.148 | 1.00 | 76.23 | A | C |
| ATOM | 1716 | CG | GLN | A | 234 | 43.846 | 65.371 | 49.461 | 1.00 | 85.96 | A | C |
| ATOM | 1717 | CD | GLN | A | 234 | 44.808 | 65.444 | 48.277 | 1.00 | 90.84 | A | C |
| ATOM | 1718 | OE1 | GLN | A | 234 | 44.574 | 64.834 | 47.229 | 1.00 | 92.67 | A | O |
| ATOM | 1719 | NE2 | GLN | A | 234 | 45.899 | 66.188 | 48.444 | 1.00 | 92.39 | A | N |
| ATOM | 1720 | C | GLN | A | 234 | 43.734 | 61.508 | 49.965 | 1.00 | 65.04 | A | C |
| ATOM | 1721 | O | GLN | A | 234 | 42.775 | 61.138 | 50.643 | 1.00 | 60.32 | A | O |
| ATOM | 1722 | N | PRO | A | 235 | 44.849 | 60.770 | 49.858 | 1.00 | 61.48 | A | N |
| ATOM | 1723 | CD | PRO | A | 235 | 46.028 | 60.985 | 49.002 | 1.00 | 61.15 | A | C |
| ATOM | 1724 | CA | PRO | A | 235 | 44.958 | 59.502 | 50.584 | 1.00 | 56.17 | A | C |
| ATOM | 1725 | CB | PRO | A | 235 | 46.369 | 59.029 | 50.244 | 1.00 | 58.10 | A | C |
| ATOM | 1726 | CG | PRO | A | 235 | 46.577 | 59.583 | 48.869 | 1.00 | 59.85 | A | C |
| ATOM | 1727 | C | PRO | A | 235 | 44.779 | 59.766 | 52.075 | 1.00 | 50.69 | A | C |
| ATOM | 1728 | O | PRO | A | 235 | 45.092 | 60.848 | 52.564 | 1.00 | 47.81 | A | O |
| ATOM | 1729 | N | ILE | A | 236 | 44.270 | 58.775 | 52.792 | 1.00 | 43.64 | A | N |
| ATOM | 1730 | CA | ILE | A | 236 | 44.039 | 58.918 | 54.215 | 1.00 | 39.82 | A | C |
| ATOM | 1731 | CB | ILE | A | 236 | 42.821 | 58.047 | 54.620 | 1.00 | 37.76 | A | C |
| ATOM | 1732 | CG2 | ILE | A | 236 | 42.696 | 57.972 | 56.132 | 1.00 | 34.22 | A | C |
| ATOM | 1733 | CG1 | ILE | A | 236 | 41.562 | 58.622 | 53.949 | 1.00 | 37.63 | A | C |
| ATOM | 1734 | CD1 | ILE | A | 236 | 40.302 | 57.754 | 54.048 | 1.00 | 34.22 | A | C |
| ATOM | 1735 | C | ILE | A | 236 | 45.271 | 58.559 | 55.056 | 1.00 | 40.12 | A | C |
| ATOM | 1736 | O | ILE | A | 236 | 45.786 | 57.440 | 54.978 | 1.00 | 38.27 | A | O |
| ATOM | 1737 | N | HIS | A | 237 | 45.742 | 59.523 | 55.851 | 1.00 | 41.02 | A | N |
| ATOM | 1738 | CA | HIS | A | 237 | 46.894 | 59.328 | 56.743 | 1.00 | 41.94 | A | C |
| ATOM | 1739 | CB | HIS | A | 237 | 48.082 | 60.226 | 56.358 | 1.00 | 45.81 | A | C |
| ATOM | 1740 | CG | HIS | A | 237 | 48.667 | 59.914 | 55.020 | 1.00 | 58.60 | A | C |
| ATOM | 1741 | CD2 | HIS | A | 237 | 49.638 | 59.043 | 54.652 | 1.00 | 62.73 | A | C |
| ATOM | 1742 | ND1 | HIS | A | 237 | 48.208 | 60.490 | 53.854 | 1.00 | 65.44 | A | N |
| ATOM | 1743 | CE1 | HIS | A | 237 | 48.868 | 59.985 | 52.826 | 1.00 | 66.88 | A | C |
| ATOM | 1744 | NE2 | HIS | A | 237 | 49.742 | 59.103 | 53.283 | 1.00 | 66.01 | A | N |
| ATOM | 1745 | C | HIS | A | 237 | 46.486 | 59.687 | 58.156 | 1.00 | 39.08 | A | C |
| ATOM | 1746 | O | HIS | A | 237 | 45.509 | 60.397 | 58.358 | 1.00 | 37.18 | A | O |
| ATOM | 1747 | N | MET | A | 238 | 47.246 | 59.208 | 59.132 | 1.00 | 36.45 | A | N |
| ATOM | 1748 | CA | MET | A | 238 | 46.965 | 59.511 | 60.527 | 1.00 | 34.02 | A | C |
| ATOM | 1749 | CB | MET | A | 238 | 45.828 | 58.626 | 61.058 | 1.00 | 37.62 | A | C |
| ATOM | 1750 | CG | MET | A | 238 | 46.238 | 57.162 | 61.304 | 1.00 | 34.75 | A | C |
| ATOM | 1751 | SD | MET | A | 238 | 44.942 | 56.127 | 62.054 | 1.00 | 37.83 | A | S |
| ATOM | 1752 | CE | MET | A | 238 | 44.761 | 56.899 | 63.659 | 1.00 | 30.36 | A | C |
| ATOM | 1753 | C | MET | A | 238 | 48.215 | 59.249 | 61.360 | 1.00 | 38.11 | A | C |
| ATOM | 1754 | O | MET | A | 238 | 49.181 | 58.632 | 60.887 | 1.00 | 39.80 | A | O |
| ATOM | 1755 | N | VAL | A | 239 | 48.197 | 59.740 | 62.594 | 1.00 | 37.24 | A | N |
| ATOM | 1756 | CA | VAL | A | 239 | 49.290 | 59.510 | 63.527 | 1.00 | 37.03 | A | C |
| ATOM | 1757 | CB | VAL | A | 239 | 49.466 | 60.698 | 64.502 | 1.00 | 36.95 | A | C |
| ATOM | 1758 | CG1 | VAL | A | 239 | 50.533 | 60.368 | 65.542 | 1.00 | 41.02 | A | C |
| ATOM | 1759 | CG2 | VAL | A | 239 | 49.861 | 61.952 | 63.736 | 1.00 | 36.85 | A | C |
| ATOM | 1760 | C | VAL | A | 239 | 48.860 | 58.272 | 64.318 | 1.00 | 35.99 | A | C |
| ATOM | 1761 | O | VAL | A | 239 | 47.825 | 58.291 | 64.990 | 1.00 | 37.09 | A | O |
| ATOM | 1762 | N | TYR | A | 240 | 49.632 | 57.195 | 64.231 | 1.00 | 37.37 | A | N |
| ATOM | 1763 | CA | TYR | A | 240 | 49.274 | 55.975 | 64.949 | 1.00 | 37.28 | A | C |
| ATOM | 1764 | CB | TYR | A | 240 | 48.086 | 55.303 | 64.234 | 1.00 | 35.97 | A | C |
| ATOM | 1765 | CG | TYR | A | 240 | 47.233 | 54.439 | 65.141 | 1.00 | 37.66 | A | C |
| ATOM | 1766 | CD1 | TYR | A | 240 | 47.259 | 53.042 | 65.052 | 1.00 | 34.69 | A | C |
| ATOM | 1767 | CE1 | TYR | A | 240 | 46.524 | 52.253 | 65.932 | 1.00 | 33.35 | A | C |
| ATOM | 1768 | CD2 | TYR | A | 240 | 46.444 | 55.019 | 66.130 | 1.00 | 32.51 | A | C |
| ATOM | 1769 | CE2 | TYR | A | 240 | 45.714 | 54.243 | 67.009 | 1.00 | 31.99 | A | C |
| ATOM | 1770 | CZ | TYR | A | 240 | 45.761 | 52.864 | 66.907 | 1.00 | 31.13 | A | C |
| ATOM | 1771 | OH | TYR | A | 240 | 45.048 | 52.121 | 67.802 | 1.00 | 35.49 | A | O |
| ATOM | 1772 | C | TYR | A | 240 | 50.437 | 54.982 | 65.073 | 1.00 | 39.73 | A | C |
| ATOM | 1773 | O | TYR | A | 240 | 51.540 | 55.229 | 64.587 | 1.00 | 38.04 | A | O |
| ATOM | 1774 | N | VAL | A | 241 | 50.176 | 53.864 | 65.744 | 1.00 | 40.01 | A | N |
| ATOM | 1775 | CA | VAL | A | 241 | 51.167 | 52.807 | 65.911 | 1.00 | 35.99 | A | C |
| ATOM | 1776 | CB | VAL | A | 241 | 51.098 | 52.173 | 67.298 | 1.00 | 38.21 | A | C |
| ATOM | 1777 | CG1 | VAL | A | 241 | 52.214 | 51.134 | 67.422 | 1.00 | 34.45 | A | C |
| ATOM | 1778 | CG2 | VAL | A | 241 | 51.174 | 53.252 | 68.388 | 1.00 | 38.29 | A | C |
| ATOM | 1779 | C | VAL | A | 241 | 50.815 | 51.715 | 64.902 | 1.00 | 36.36 | A | C |
| ATOM | 1780 | O | VAL | A | 241 | 49.981 | 50.843 | 65.181 | 1.00 | 32.97 | A | O |
| ATOM | 1781 | N | PRO | A | 242 | 51.466 | 51.725 | 63.729 | 1.00 | 34.40 | A | N |
| ATOM | 1782 | CD | PRO | A | 242 | 52.515 | 52.646 | 63.261 | 1.00 | 35.87 | A | C |
| ATOM | 1783 | CA | PRO | A | 242 | 51.159 | 50.711 | 62.714 | 1.00 | 35.94 | A | C |
| ATOM | 1784 | CB | PRO | A | 242 | 52.160 | 51.023 | 61.588 | 1.00 | 35.32 | A | C |
| ATOM | 1785 | CG | PRO | A | 242 | 53.250 | 51.803 | 62.262 | 1.00 | 34.85 | A | C |
| ATOM | 1786 | C | PRO | A | 242 | 51.128 | 49.230 | 63.130 | 1.00 | 36.16 | A | C |
| ATOM | 1787 | O | PRO | A | 242 | 50.317 | 48.469 | 62.608 | 1.00 | 31.50 | A | O |
| ATOM | 1788 | N | SER | A | 243 | 51.975 | 48.812 | 64.065 | 1.00 | 35.45 | A | N |
| ATOM | 1789 | CA | SER | A | 243 | 51.953 | 47.406 | 64.469 | 1.00 | 37.51 | A | C |
| ATOM | 1790 | CB | SER | A | 243 | 53.196 | 47.049 | 65.310 | 1.00 | 37.41 | A | C |
| ATOM | 1791 | OG | SER | A | 243 | 53.174 | 47.657 | 66.586 | 1.00 | 41.02 | A | O |
| ATOM | 1792 | C | SER | A | 243 | 50.663 | 47.094 | 65.243 | 1.00 | 35.91 | A | C |
| ATOM | 1793 | O | SER | A | 243 | 50.138 | 45.986 | 65.171 | 1.00 | 33.67 | A | O |
| ATOM | 1794 | N | HIS | A | 244 | 50.159 | 48.078 | 65.980 | 1.00 | 35.09 | A | N |
| ATOM | 1795 | CA | HIS | A | 244 | 48.913 | 47.909 | 66.722 | 1.00 | 35.98 | A | C |
| ATOM | 1796 | CB | HIS | A | 244 | 48.609 | 49.160 | 67.535 | 1.00 | 36.90 | A | C |
| ATOM | 1797 | CG | HIS | A | 244 | 49.298 | 49.227 | 68.866 | 1.00 | 41.22 | A | C |
| ATOM | 1798 | CD2 | HIS | A | 244 | 48.932 | 49.842 | 70.016 | 1.00 | 37.46 | A | C |
| ATOM | 1799 | ND1 | HIS | A | 244 | 50.539 | 48.677 | 69.106 | 1.00 | 45.88 | A | N |
| ATOM | 1800 | CE1 | HIS | A | 244 | 50.907 | 48.948 | 70.349 | 1.00 | 38.08 | A | C |
| ATOM | 1801 | NE2 | HIS | A | 244 | 49.950 | 49.655 | 70.920 | 1.00 | 41.27 | A | N |
| ATOM | 1802 | C | HIS | A | 244 | 47.767 | 47.699 | 65.716 | 1.00 | 37.78 | A | C |
| ATOM | 1803 | O | HIS | A | 244 | 46.944 | 46.789 | 65.870 | 1.00 | 36.00 | A | O |
| ATOM | 1804 | N | LEU | A | 245 | 47.727 | 48.552 | 64.691 | 1.00 | 34.19 | A | N |
| ATOM | 1805 | CA | LEU | A | 245 | 46.685 | 48.498 | 63.671 | 1.00 | 36.57 | A | C |
| ATOM | 1806 | CB | LEU | A | 245 | 46.815 | 49.700 | 62.721 | 1.00 | 33.81 | A | C |
| ATOM | 1807 | CG | LEU | A | 245 | 45.835 | 49.749 | 61.537 | 1.00 | 36.57 | A | C |
| ATOM | 1808 | CD1 | LEU | A | 245 | 44.396 | 49.789 | 62.051 | 1.00 | 34.91 | A | C |
| ATOM | 1809 | CD2 | LEU | A | 245 | 46.115 | 50.972 | 60.678 | 1.00 | 33.45 | A | C |
| ATOM | 1810 | C | LEU | A | 245 | 46.779 | 47.192 | 62.886 | 1.00 | 37.13 | A | C |
| ATOM | 1811 | O | LEU | A | 245 | 45.766 | 46.590 | 62.524 | 1.00 | 35.79 | A | O |
| ATOM | 1812 | N | TYR | A | 246 | 48.010 | 46.758 | 62.637 | 1.00 | 38.57 | A | N |
| ATOM | 1813 | CA | TYR | A | 246 | 48.271 | 45.521 | 61.904 | 1.00 | 39.22 | A | C |
| ATOM | 1814 | CB | TYR | A | 246 | 49.782 | 45.309 | 61.771 | 1.00 | 39.31 | A | C |
| ATOM | 1815 | CG | TYR | A | 246 | 50.141 | 44.040 | 61.048 | 1.00 | 41.30 | A | C |
| ATOM | 1816 | CD1 | TYR | A | 246 | 50.345 | 44.034 | 59.667 | 1.00 | 36.46 | A | C |
| ATOM | 1817 | CE1 | TYR | A | 246 | 50.599 | 42.854 | 58.989 | 1.00 | 41.41 | A | C |
| ATOM | 1818 | CD2 | TYR | A | 246 | 50.206 | 42.830 | 61.733 | 1.00 | 38.34 | A | C |
| ATOM | 1819 | CE2 | TYR | A | 246 | 50.457 | 41.642 | 61.067 | 1.00 | 39.50 | A | C |
| ATOM | 1820 | CZ | TYR | A | 246 | 50.652 | 41.658 | 59.697 | 1.00 | 43.26 | A | C |
| ATOM | 1821 | OH | TYR | A | 246 | 50.886 | 40.476 | 59.036 | 1.00 | 38.55 | A | O |
| ATOM | 1822 | C | TYR | A | 246 | 47.655 | 44.328 | 62.642 | 1.00 | 40.03 | A | C |
| ATOM | 1823 | O | TYR | A | 246 | 46.928 | 43.516 | 62.057 | 1.00 | 40.70 | A | O |
| ATOM | 1824 | N | HIS | A | 247 | 47.954 | 44.239 | 63.933 | 1.00 | 37.04 | A | N |
| ATOM | 1825 | CA | HIS | A | 247 | 47.465 | 43.155 | 64.775 | 1.00 | 37.63 | A | C |
| ATOM | 1826 | CB | HIS | A | 247 | 47.934 | 43.393 | 66.221 | 1.00 | 43.69 | A | C |
| ATOM | 1827 | CG | HIS | A | 247 | 47.558 | 42.304 | 67.180 | 1.00 | 55.31 | A | C |
| ATOM | 1828 | CD2 | HIS | A | 247 | 46.377 | 41.673 | 67.405 | 1.00 | 60.10 | A | C |
| ATOM | 1829 | ND1 | HIS | A | 247 | 48.461 | 41.748 | 68.065 | 1.00 | 60.02 | A | N |
| ATOM | 1830 | CE1 | HIS | A | 247 | 47.855 | 40.821 | 68.788 | 1.00 | 61.57 | A | C |
| ATOM | 1831 | NE2 | HIS | A | 247 | 46.590 | 40.756 | 68.408 | 1.00 | 62.13 | A | N |
| ATOM | 1832 | C | HIS | A | 247 | 45.941 | 43.055 | 64.713 | 1.00 | 36.38 | A | C |
| ATOM | 1833 | O | HIS | A | 247 | 45.378 | 41.966 | 64.529 | 1.00 | 33.10 | A | O |
| ATOM | 1834 | N | MET | A | 248 | 45.272 | 44.190 | 64.875 | 1.00 | 30.30 | A | N |
| ATOM | 1835 | CA | MET | A | 248 | 43.815 | 44.198 | 64.844 | 1.00 | 32.36 | A | C |
| ATOM | 1836 | CB | MET | A | 248 | 43.283 | 45.612 | 65.120 | 1.00 | 33.09 | A | C |
| ATOM | 1837 | CG | MET | A | 248 | 43.563 | 46.141 | 66.519 | 1.00 | 32.34 | A | C |
| ATOM | 1838 | SD | MET | A | 248 | 42.692 | 47.718 | 66.827 | 1.00 | 36.22 | A | S |
| ATOM | 1839 | CE | MET | A | 248 | 43.776 | 48.859 | 66.060 | 1.00 | 22.20 | A | C |
| ATOM | 1840 | C | MET | A | 248 | 43.294 | 43.704 | 63.491 | 1.00 | 28.41 | A | C |
| ATOM | 1841 | O | MET | A | 248 | 42.460 | 42.807 | 63.431 | 1.00 | 33.50 | A | O |
| ATOM | 1842 | N | LEU | A | 249 | 43.799 | 44.282 | 62.408 | 1.00 | 30.13 | A | N |
| ATOM | 1843 | CA | LEU | A | 249 | 43.360 | 43.909 | 61.068 | 1.00 | 31.68 | A | C |
| ATOM | 1844 | CB | LEU | A | 249 | 43.976 | 44.859 | 60.036 | 1.00 | 28.37 | A | C |
| ATOM | 1845 | CG | LEU | A | 249 | 43.354 | 46.264 | 59.963 | 1.00 | 34.82 | A | C |
| ATOM | 1846 | CD1 | LEU | A | 249 | 44.218 | 47.187 | 59.105 | 1.00 | 26.23 | A | C |
| ATOM | 1847 | CD2 | LEU | A | 249 | 41.941 | 46.155 | 59.380 | 1.00 | 28.70 | A | C |
| ATOM | 1848 | C | LEU | A | 249 | 43.669 | 42.464 | 60.674 | 1.00 | 35.31 | A | C |
| ATOM | 1849 | O | LEU | A | 249 | 42.889 | 41.830 | 59.953 | 1.00 | 36.34 | A | O |
| ATOM | 1850 | N | PHE | A | 250 | 44.802 | 41.943 | 61.132 | 1.00 | 34.58 | A | N |
| ATOM | 1851 | CA | PHE | A | 250 | 45.186 | 40.570 | 60.797 | 1.00 | 34.44 | A | C |
| ATOM | 1852 | CB | PHE | A | 250 | 46.622 | 40.289 | 61.286 | 1.00 | 37.88 | A | C |
| ATOM | 1853 | CG | PHE | A | 250 | 47.098 | 38.884 | 61.013 | 1.00 | 42.29 | A | C |
| ATOM | 1854 | CD1 | PHE | A | 250 | 47.253 | 38.425 | 59.704 | 1.00 | 42.28 | A | C |
| ATOM | 1855 | CD2 | PHE | A | 250 | 47.372 | 38.016 | 62.066 | 1.00 | 44.62 | A | C |
| ATOM | 1856 | CE1 | PHE | A | 250 | 47.672 | 37.117 | 59.444 | 1.00 | 44.14 | A | C |
| ATOM | 1857 | CE2 | PHE | A | 250 | 47.797 | 36.696 | 61.822 | 1.00 | 48.65 | A | C |
| ATOM | 1858 | CZ | PHE | A | 250 | 47.945 | 36.249 | 60.508 | 1.00 | 46.83 | A | C |
| ATOM | 1859 | C | PHE | A | 250 | 44.197 | 39.601 | 61.452 | 1.00 | 30.78 | A | C |
| ATOM | 1860 | O | PHE | A | 250 | 43.765 | 38.620 | 60.842 | 1.00 | 30.64 | A | O |
| ATOM | 1861 | N | GLU | A | 251 | 43.853 | 39.876 | 62.703 | 1.00 | 30.59 | A | N |
| ATOM | 1862 | CA | GLU | A | 251 | 42.910 | 39.033 | 63.417 | 1.00 | 30.94 | A | C |
| ATOM | 1863 | CB | GLU | A | 251 | 42.669 | 39.576 | 64.823 | 1.00 | 36.58 | A | C |
| ATOM | 1864 | CG | GLU | A | 251 | 41.560 | 38.844 | 65.552 | 1.00 | 44.41 | A | C |
| ATOM | 1865 | CD | GLU | A | 251 | 41.895 | 37.376 | 65.776 | 1.00 | 51.44 | A | C |
| ATOM | 1866 | OE1 | GLU | A | 251 | 40.960 | 36.567 | 65.988 | 1.00 | 50.81 | A | O |
| ATOM | 1867 | OE2 | GLU | A | 251 | 43.101 | 37.038 | 65.751 | 1.00 | 51.21 | A | O |
| ATOM | 1868 | C | GLU | A | 251 | 41.581 | 39.002 | 62.643 | 1.00 | 32.20 | A | C |
| ATOM | 1869 | O | GLU | A | 251 | 41.037 | 37.926 | 62.391 | 1.00 | 30.16 | A | O |
| ATOM | 1870 | N | LEU | A | 252 | 41.084 | 40.181 | 62.258 | 1.00 | 26.74 | A | N |
| ATOM | 1871 | CA | LEU | A | 252 | 39.819 | 40.290 | 61.535 | 1.00 | 29.03 | A | C |
| ATOM | 1872 | CB | LEU | A | 252 | 39.412 | 41.773 | 61.395 | 1.00 | 22.72 | A | C |
| ATOM | 1873 | CG | LEU | A | 252 | 39.074 | 42.459 | 62.729 | 1.00 | 23.59 | A | C |
| ATOM | 1874 | CD1 | LEU | A | 252 | 38.701 | 43.916 | 62.500 | 1.00 | 23.18 | A | C |
| ATOM | 1875 | CD2 | LEU | A | 252 | 37.911 | 41.739 | 63.383 | 1.00 | 19.96 | A | C |
| ATOM | 1876 | C | LEU | A | 252 | 39.865 | 39.598 | 60.163 | 1.00 | 30.06 | A | C |
| ATOM | 1877 | O | LEU | A | 252 | 38.951 | 38.857 | 59.823 | 1.00 | 27.02 | A | O |
| ATOM | 1878 | N | PHE | A | 253 | 40.917 | 39.833 | 59.374 | 1.00 | 28.29 | A | N |
| ATOM | 1879 | CA | PHE | A | 253 | 41.024 | 39.173 | 58.077 | 1.00 | 30.28 | A | C |
| ATOM | 1880 | CB | PHE | A | 253 | 42.312 | 39.583 | 57.351 | 1.00 | 32.01 | A | C |
| ATOM | 1881 | CG | PHE | A | 253 | 42.172 | 40.829 | 56.531 | 1.00 | 29.45 | A | C |
| ATOM | 1882 | CD1 | PHE | A | 253 | 41.191 | 40.922 | 55.549 | 1.00 | 28.25 | A | C |
| ATOM | 1883 | CD2 | PHE | A | 253 | 43.029 | 41.906 | 56.731 | 1.00 | 27.98 | A | C |
| ATOM | 1884 | CE1 | PHE | A | 253 | 41.065 | 42.074 | 54.774 | 1.00 | 30.18 | A | C |
| ATOM | 1885 | CE2 | PHE | A | 253 | 42.912 | 43.066 | 55.963 | 1.00 | 25.37 | A | C |
| ATOM | 1886 | CZ | PHE | A | 253 | 41.928 | 43.150 | 54.981 | 1.00 | 28.04 | A | C |
| ATOM | 1887 | C | PHE | A | 253 | 40.989 | 37.647 | 58.203 | 1.00 | 33.33 | A | C |
| ATOM | 1888 | O | PHE | A | 253 | 40.286 | 36.975 | 57.437 | 1.00 | 35.35 | A | O |
| ATOM | 1889 | N | LYS | A | 254 | 41.734 | 37.097 | 59.163 | 1.00 | 34.15 | A | N |
| ATOM | 1890 | CA | LYS | A | 254 | 41.766 | 35.646 | 59.358 | 1.00 | 32.06 | A | C |
| ATOM | 1891 | CB | LYS | A | 254 | 42.673 | 35.243 | 60.523 | 1.00 | 37.16 | A | C |
| ATOM | 1892 | CG | LYS | A | 254 | 44.163 | 35.355 | 60.215 | 1.00 | 44.24 | A | C |
| ATOM | 1893 | CD | LYS | A | 254 | 45.045 | 34.709 | 61.313 | 1.00 | 51.30 | A | C |
| ATOM | 1894 | CE | LYS | A | 254 | 44.885 | 35.379 | 62.691 | 1.00 | 51.55 | A | C |
| ATOM | 1895 | NZ | LYS | A | 254 | 45.666 | 34.695 | 63.779 | 1.00 | 51.24 | A | N |
| ATOM | 1896 | C | LYS | A | 254 | 40.384 | 35.087 | 59.629 | 1.00 | 36.38 | A | C |
| ATOM | 1897 | O | LYS | A | 254 | 40.027 | 34.024 | 59.103 | 1.00 | 32.63 | A | O |
| ATOM | 1898 | N | ASN | A | 255 | 39.607 | 35.777 | 60.465 | 1.00 | 32.89 | A | N |
| ATOM | 1899 | CA | ASN | A | 255 | 38.268 | 35.293 | 60.774 | 1.00 | 31.40 | A | C |
| ATOM | 1900 | CB | ASN | A | 255 | 37.650 | 36.087 | 61.939 | 1.00 | 36.22 | A | C |
| ATOM | 1901 | CG | ASN | A | 255 | 38.409 | 35.891 | 63.245 | 1.00 | 39.27 | A | C |
| ATOM | 1902 | OD1 | ASN | A | 255 | 38.894 | 34.798 | 63.529 | 1.00 | 39.98 | A | O |
| ATOM | 1903 | ND2 | ASN | A | 255 | 38.502 | 36.944 | 64.049 | 1.00 | 34.30 | A | N |
| ATOM | 1904 | C | ASN | A | 255 | 37.379 | 35.415 | 59.541 | 1.00 | 29.42 | A | C |
| ATOM | 1905 | O | ASN | A | 255 | 36.634 | 34.494 | 59.206 | 1.00 | 30.20 | A | O |
| ATOM | 1906 | N | ALA | A | 256 | 37.464 | 36.557 | 58.864 | 1.00 | 25.84 | A | N |
| ATOM | 1907 | CA | ALA | A | 256 | 36.655 | 36.788 | 57.677 | 1.00 | 29.06 | A | C |
| ATOM | 1908 | CB | ALA | A | 256 | 36.838 | 38.220 | 57.210 | 1.00 | 28.09 | A | C |
| ATOM | 1909 | C | ALA | A | 256 | 37.020 | 35.804 | 56.537 | 1.00 | 34.62 | A | C |
| ATOM | 1910 | O | ALA | A | 256 | 36.153 | 35.398 | 55.751 | 1.00 | 33.46 | A | O |
| ATOM | 1911 | N | MET | A | 257 | 38.290 | 35.411 | 56.445 | 1.00 | 31.51 | A | N |
| ATOM | 1912 | CA | MET | A | 257 | 38.680 | 34.488 | 55.376 | 1.00 | 33.49 | A | C |
| ATOM | 1913 | CB | MET | A | 257 | 40.202 | 34.451 | 55.200 | 1.00 | 32.29 | A | C |
| ATOM | 1914 | CG | MET | A | 257 | 40.785 | 35.778 | 54.742 | 1.00 | 40.04 | A | C |
| ATOM | 1915 | SD | MET | A | 257 | 42.531 | 35.712 | 54.223 | 1.00 | 45.10 | A | S |
| ATOM | 1916 | CE | MET | A | 257 | 42.625 | 37.281 | 53.329 | 1.00 | 37.38 | A | C |
| ATOM | 1917 | C | MET | A | 257 | 38.161 | 33.089 | 55.664 | 1.00 | 32.34 | A | C |
| ATOM | 1918 | O | MET | A | 257 | 37.529 | 32.475 | 54.814 | 1.00 | 36.96 | A | O |
| ATOM | 1919 | N | ARG | A | 258 | 38.411 | 32.606 | 56.873 | 1.00 | 31.51 | A | N |
| ATOM | 1920 | CA | ARG | A | 258 | 37.975 | 31.281 | 57.283 | 1.00 | 34.89 | A | C |
| ATOM | 1921 | CB | ARG | A | 258 | 38.385 | 31.058 | 58.744 | 1.00 | 35.82 | A | C |
| ATOM | 1922 | CG | ARG | A | 258 | 37.693 | 29.922 | 59.480 | 1.00 | 49.83 | A | C |
| ATOM | 1923 | CD | ARG | A | 258 | 38.258 | 29.821 | 60.902 | 1.00 | 60.74 | A | C |
| ATOM | 1924 | NE | ARG | A | 258 | 37.337 | 29.219 | 61.870 | 1.00 | 72.39 | A | N |
| ATOM | 1925 | CZ | ARG | A | 258 | 37.061 | 27.918 | 61.957 | 1.00 | 78.52 | A | C |
| ATOM | 1926 | NH1 | ARG | A | 258 | 37.636 | 27.056 | 61.126 | 1.00 | 82.00 | A | N |
| ATOM | 1927 | NH2 | ARG | A | 258 | 36.208 | 27.478 | 62.879 | 1.00 | 78.73 | A | N |
| ATOM | 1928 | C | ARG | A | 258 | 36.461 | 31.110 | 57.106 | 1.00 | 39.04 | A | C |
| ATOM | 1929 | O | ARG | A | 258 | 36.002 | 30.095 | 56.580 | 1.00 | 38.56 | A | O |
| ATOM | 1930 | N | ALA | A | 259 | 35.687 | 32.101 | 57.541 | 1.00 | 35.38 | A | N |
| ATOM | 1931 | CA | ALA | A | 259 | 34.238 | 32.021 | 57.422 | 1.00 | 33.86 | A | C |
| ATOM | 1932 | CB | ALA | A | 259 | 33.572 | 33.199 | 58.147 | 1.00 | 32.94 | A | C |
| ATOM | 1933 | C | ALA | A | 259 | 33.800 | 31.999 | 55.970 | 1.00 | 33.26 | A | C |
| ATOM | 1934 | O | ALA | A | 259 | 32.904 | 31.243 | 55.608 | 1.00 | 31.51 | A | O |
| ATOM | 1935 | N | THR | A | 260 | 34.418 | 32.837 | 55.142 | 1.00 | 32.33 | A | N |
| ATOM | 1936 | CA | THR | A | 260 | 34.054 | 32.895 | 53.733 | 1.00 | 36.96 | A | C |
| ATOM | 1937 | CB | THR | A | 260 | 34.797 | 34.033 | 53.006 | 1.00 | 35.95 | A | C |
| ATOM | 1938 | OG1 | THR | A | 260 | 34.509 | 35.277 | 53.652 | 1.00 | 35.42 | A | O |
| ATOM | 1939 | CG2 | THR | A | 260 | 34.364 | 34.106 | 51.546 | 1.00 | 31.10 | A | C |
| ATOM | 1940 | C | THR | A | 260 | 34.388 | 31.565 | 53.041 | 1.00 | 42.84 | A | C |
| ATOM | 1941 | O | THR | A | 260 | 33.572 | 31.028 | 52.278 | 1.00 | 40.66 | A | O |
| ATOM | 1942 | N | VAL | A | 261 | 35.580 | 31.038 | 53.326 | 1.00 | 42.75 | A | N |
| ATOM | 1943 | CA | VAL | A | 261 | 36.035 | 29.776 | 52.743 | 1.00 | 44.77 | A | C |
| ATOM | 1944 | CB | VAL | A | 261 | 37.522 | 29.478 | 53.138 | 1.00 | 46.75 | A | C |
| ATOM | 1945 | CG1 | VAL | A | 261 | 37.862 | 28.016 | 52.872 | 1.00 | 50.09 | A | C |
| ATOM | 1946 | CG2 | VAL | A | 261 | 38.464 | 30.368 | 52.330 | 1.00 | 46.81 | A | C |
| ATOM | 1947 | C | VAL | A | 261 | 35.149 | 28.602 | 53.165 | 1.00 | 41.39 | A | C |
| ATOM | 1948 | O | VAL | A | 261 | 34.543 | 27.932 | 52.327 | 1.00 | 42.46 | A | O |
| ATOM | 1949 | N | GLU | A | 262 | 35.067 | 28.358 | 54.463 | 1.00 | 40.94 | A | N |
| ATOM | 1950 | CA | GLU | A | 262 | 34.277 | 27.251 | 54.968 | 1.00 | 41.08 | A | C |
| ATOM | 1951 | CB | GLU | A | 262 | 34.475 | 27.134 | 56.475 | 1.00 | 43.13 | A | C |
| ATOM | 1952 | CG | GLU | A | 262 | 35.931 | 26.874 | 56.871 | 1.00 | 45.24 | A | C |
| ATOM | 1953 | CD | GLU | A | 262 | 36.100 | 26.581 | 58.352 | 1.00 | 48.22 | A | C |
| ATOM | 1954 | OE1 | GLU | A | 262 | 37.211 | 26.167 | 58.761 | 1.00 | 51.02 | A | O |
| ATOM | 1955 | OE2 | GLU | A | 262 | 35.123 | 26.766 | 59.112 | 1.00 | 50.71 | A | O |
| ATOM | 1956 | C | GLU | A | 262 | 32.786 | 27.312 | 54.619 | 1.00 | 44.62 | A | C |
| ATOM | 1957 | O | GLU | A | 262 | 32.152 | 26.269 | 54.430 | 1.00 | 43.47 | A | O |
| ATOM | 1958 | N | SER | A | 263 | 32.224 | 28.514 | 54.506 | 1.00 | 41.98 | A | N |
| ATOM | 1959 | CA | SER | A | 263 | 30.803 | 28.636 | 54.177 | 1.00 | 41.66 | A | C |
| ATOM | 1960 | CB | SER | A | 263 | 30.253 | 29.992 | 54.651 | 1.00 | 41.56 | A | C |
| ATOM | 1961 | OG | SER | A | 263 | 30.671 | 31.051 | 53.800 | 1.00 | 40.47 | A | O |
| ATOM | 1962 | C | SER | A | 263 | 30.523 | 28.473 | 52.680 | 1.00 | 43.48 | A | C |
| ATOM | 1963 | O | SER | A | 263 | 29.368 | 28.459 | 52.262 | 1.00 | 41.76 | A | O |
| ATOM | 1964 | N | HIS | A | 264 | 31.568 | 28.379 | 51.864 | 1.00 | 44.78 | A | N |
| ATOM | 1965 | CA | HIS | A | 264 | 31.372 | 28.202 | 50.424 | 1.00 | 48.63 | A | C |
| ATOM | 1966 | CB | HIS | A | 264 | 31.875 | 29.417 | 49.650 | 1.00 | 43.82 | A | C |
| ATOM | 1967 | CG | HIS | A | 264 | 31.032 | 30.635 | 49.825 | 1.00 | 46.10 | A | C |
| ATOM | 1968 | CD2 | HIS | A | 264 | 30.225 | 31.291 | 48.957 | 1.00 | 45.13 | A | C |
| ATOM | 1969 | ND1 | HIS | A | 264 | 30.978 | 31.342 | 51.008 | 1.00 | 47.74 | A | N |
| ATOM | 1970 | CE1 | HIS | A | 264 | 30.178 | 32.383 | 50.859 | 1.00 | 44.30 | A | C |
| ATOM | 1971 | NE2 | HIS | A | 264 | 29.708 | 32.375 | 49.624 | 1.00 | 47.32 | A | N |
| ATOM | 1972 | C | HIS | A | 264 | 32.073 | 26.957 | 49.879 | 1.00 | 52.53 | A | C |
| ATOM | 1973 | O | HIS | A | 264 | 32.404 | 26.890 | 48.695 | 1.00 | 53.80 | A | O |
| ATOM | 1974 | N | GLU | A | 265 | 32.300 | 25.971 | 50.735 | 1.00 | 55.39 | A | N |
| ATOM | 1975 | CA | GLU | A | 265 | 32.970 | 24.763 | 50.293 | 1.00 | 63.83 | A | C |
| ATOM | 1976 | CB | GLU | A | 265 | 33.246 | 23.856 | 51.483 | 1.00 | 67.50 | A | C |
| ATOM | 1977 | CG | GLU | A | 265 | 34.097 | 24.533 | 52.527 | 1.00 | 74.88 | A | C |
| ATOM | 1978 | CD | GLU | A | 265 | 34.726 | 23.555 | 53.482 | 1.00 | 79.49 | A | C |
| ATOM | 1979 | OE1 | GLU | A | 265 | 33.994 | 22.674 | 53.983 | 1.00 | 82.65 | A | O |
| ATOM | 1980 | OE2 | GLU | A | 265 | 35.948 | 23.673 | 53.733 | 1.00 | 81.31 | A | O |
| ATOM | 1981 | C | GLU | A | 265 | 32.177 | 24.012 | 49.236 | 1.00 | 66.13 | A | C |
| ATOM | 1982 | O | GLU | A | 265 | 32.483 | 22.862 | 48.929 | 1.00 | 66.97 | A | O |
| ATOM | 1983 | N | SER | A | 266 | 31.167 | 24.674 | 48.678 | 1.00 | 67.20 | A | N |
| ATOM | 1984 | CA | SER | A | 266 | 30.331 | 24.081 | 47.650 | 1.00 | 69.31 | A | C |
| ATOM | 1985 | CB | SER | A | 266 | 29.075 | 23.469 | 48.277 | 1.00 | 69.06 | A | C |
| ATOM | 1986 | OG | SER | A | 266 | 29.404 | 22.423 | 49.179 | 1.00 | 70.09 | A | O |
| ATOM | 1987 | C | SER | A | 266 | 29.925 | 25.107 | 46.599 | 1.00 | 72.00 | A | C |
| ATOM | 1988 | O | SER | A | 266 | 28.777 | 25.109 | 46.161 | 1.00 | 73.49 | A | O |
| ATOM | 1989 | N | SER | A | 267 | 30.855 | 25.969 | 46.186 | 1.00 | 73.84 | A | N |
| ATOM | 1990 | CA | SER | A | 267 | 30.537 | 26.980 | 45.174 | 1.00 | 77.14 | A | C |
| ATOM | 1991 | CB | SER | A | 267 | 29.379 | 27.855 | 45.673 | 1.00 | 76.49 | A | C |
| ATOM | 1992 | OG | SER | A | 267 | 29.578 | 28.242 | 47.018 | 1.00 | 74.73 | A | O |
| ATOM | 1993 | C | SER | A | 267 | 31.714 | 27.866 | 44.740 | 1.00 | 79.49 | A | C |
| ATOM | 1994 | O | SER | A | 267 | 32.825 | 27.377 | 44.541 | 1.00 | 81.43 | A | O |
| ATOM | 1995 | N | LEU | A | 268 | 31.453 | 29.164 | 44.573 | 1.00 | 82.57 | A | N |
| ATOM | 1996 | CA | LEU | A | 268 | 32.478 | 30.138 | 44.179 | 1.00 | 82.37 | A | C |
| ATOM | 1997 | CB | LEU | A | 268 | 31.847 | 31.281 | 43.373 | 1.00 | 84.32 | A | C |
| ATOM | 1998 | CG | LEU | A | 268 | 30.867 | 32.229 | 44.074 | 1.00 | 86.56 | A | C |
| ATOM | 1999 | CD1 | LEU | A | 268 | 30.477 | 33.338 | 43.114 | 1.00 | 87.80 | A | C |
| ATOM | 2000 | CD2 | LEU | A | 268 | 29.637 | 31.472 | 44.550 | 1.00 | 88.71 | A | C |
| ATOM | 2001 | C | LEU | A | 268 | 33.202 | 30.701 | 45.422 | 1.00 | 80.27 | A | C |
| ATOM | 2002 | O | LEU | A | 268 | 33.721 | 29.923 | 46.230 | 1.00 | 83.08 | A | O |
| ATOM | 2003 | N | ILE | A | 269 | 33.240 | 32.029 | 45.584 | 1.00 | 72.50 | A | N |
| ATOM | 2004 | CA | ILE | A | 269 | 33.925 | 32.640 | 46.737 | 1.00 | 64.06 | A | C |
| ATOM | 2005 | CB | ILE | A | 269 | 35.360 | 32.052 | 46.863 | 1.00 | 62.66 | A | C |
| ATOM | 2006 | CG2 | ILE | A | 269 | 36.144 | 32.312 | 45.585 | 1.00 | 67.71 | A | C |
| ATOM | 2007 | CG1 | ILE | A | 269 | 36.076 | 32.613 | 48.084 | 1.00 | 61.74 | A | C |
| ATOM | 2008 | CD1 | ILE | A | 269 | 37.379 | 31.884 | 48.387 | 1.00 | 56.02 | A | C |
| ATOM | 2009 | C | ILE | A | 269 | 33.975 | 34.181 | 46.642 | 1.00 | 57.46 | A | C |
| ATOM | 2010 | O | ILE | A | 269 | 34.800 | 34.751 | 45.930 | 1.00 | 57.22 | A | O |
| ATOM | 2011 | N | PRO | A | 271 | 33.082 | 34.875 | 47.370 | 1.00 | 51.25 | A | N |
| ATOM | 2012 | CD | PRO | A | 271 | 32.031 | 34.345 | 48.257 | 1.00 | 49.93 | A | C |
| ATOM | 2013 | CA | PRO | A | 271 | 33.049 | 36.342 | 47.337 | 1.00 | 45.68 | A | C |
| ATOM | 2014 | CB | PRO | A | 271 | 31.718 | 36.669 | 48.000 | 1.00 | 45.49 | A | C |
| ATOM | 2015 | CG | PRO | A | 271 | 31.579 | 35.579 | 48.997 | 1.00 | 47.15 | A | C |
| ATOM | 2016 | C | PRO | A | 271 | 34.229 | 37.017 | 48.029 | 1.00 | 40.42 | A | C |
| ATOM | 2017 | O | PRO | A | 271 | 34.895 | 36.424 | 48.877 | 1.00 | 40.38 | A | O |
| ATOM | 2018 | N | PRO | A | 272 | 34.493 | 38.275 | 47.678 | 1.00 | 35.31 | A | N |
| ATOM | 2019 | CD | PRO | A | 272 | 33.810 | 39.034 | 46.612 | 1.00 | 37.13 | A | C |
| ATOM | 2020 | CA | PRO | A | 272 | 35.592 | 39.051 | 48.243 | 1.00 | 35.22 | A | C |
| ATOM | 2021 | CB | PRO | A | 272 | 35.882 | 40.057 | 47.139 | 1.00 | 38.43 | A | C |
| ATOM | 2022 | CG | PRO | A | 272 | 34.493 | 40.404 | 46.676 | 1.00 | 38.02 | A | C |
| ATOM | 2023 | C | PRO | A | 272 | 35.293 | 39.753 | 49.565 | 1.00 | 36.95 | A | C |
| ATOM | 2024 | O | PRO | A | 272 | 34.132 | 39.986 | 49.919 | 1.00 | 37.84 | A | O |
| ATOM | 2025 | N | ILE | A | 273 | 36.359 | 40.092 | 50.282 | 1.00 | 33.55 | A | N |
| ATOM | 2026 | CA | ILE | A | 273 | 36.252 | 40.820 | 51.541 | 1.00 | 30.96 | A | C |
| ATOM | 2027 | CB | ILE | A | 273 | 37.372 | 40.400 | 52.497 | 1.00 | 27.84 | A | C |
| ATOM | 2028 | CG2 | ILE | A | 273 | 37.381 | 41.283 | 53.739 | 1.00 | 25.35 | A | C |
| ATOM | 2029 | CG1 | ILE | A | 273 | 37.135 | 38.945 | 52.911 | 1.00 | 27.30 | A | C |
| ATOM | 2030 | CD1 | ILE | A | 273 | 38.200 | 38.353 | 53.784 | 1.00 | 30.38 | A | C |
| ATOM | 2031 | C | ILE | A | 273 | 36.389 | 42.286 | 51.155 | 1.00 | 31.33 | A | C |
| ATOM | 2032 | O | ILE | A | 273 | 37.343 | 42.668 | 50.475 | 1.00 | 34.38 | A | O |
| ATOM | 2033 | N | LYS | A | 274 | 35.427 | 43.108 | 51.554 | 1.00 | 30.90 | A | N |
| ATOM | 2034 | CA | LYS | A | 274 | 35.474 | 44.528 | 51.214 | 1.00 | 30.71 | A | C |
| ATOM | 2035 | CB | LYS | A | 274 | 34.146 | 44.972 | 50.603 | 1.00 | 31.37 | A | C |
| ATOM | 2036 | CG | LYS | A | 274 | 33.606 | 43.980 | 49.579 | 1.00 | 36.66 | A | C |
| ATOM | 2037 | CD | LYS | A | 274 | 32.429 | 44.550 | 48.797 | 1.00 | 44.61 | A | C |
| ATOM | 2038 | CE | LYS | A | 274 | 31.968 | 43.555 | 47.744 | 1.00 | 50.39 | A | C |
| ATOM | 2039 | NZ | LYS | A | 274 | 31.221 | 44.236 | 46.650 | 1.00 | 57.48 | A | N |
| ATOM | 2040 | C | LYS | A | 274 | 35.786 | 45.379 | 52.440 | 1.00 | 35.18 | A | C |
| ATOM | 2041 | O | LYS | A | 274 | 35.212 | 45.189 | 53.522 | 1.00 | 36.29 | A | O |
| ATOM | 2042 | N | VAL | A | 275 | 36.695 | 46.326 | 52.266 | 1.00 | 29.59 | A | N |
| ATOM | 2043 | CA | VAL | A | 275 | 37.093 | 47.191 | 53.362 | 1.00 | 29.52 | A | C |
| ATOM | 2044 | CB | VAL | A | 275 | 38.594 | 47.020 | 53.695 | 1.00 | 27.06 | A | C |
| ATOM | 2045 | CG1 | VAL | A | 275 | 39.033 | 48.071 | 54.694 | 1.00 | 20.93 | A | C |
| ATOM | 2046 | CG2 | VAL | A | 275 | 38.843 | 45.640 | 54.249 | 1.00 | 26.08 | A | C |
| ATOM | 2047 | C | VAL | A | 275 | 36.869 | 48.642 | 53.006 | 1.00 | 30.85 | A | C |
| ATOM | 2048 | O | VAL | A | 275 | 37.159 | 49.075 | 51.888 | 1.00 | 32.33 | A | O |
| ATOM | 2049 | N | MET | A | 276 | 36.342 | 49.400 | 53.951 | 1.00 | 29.12 | A | N |
| ATOM | 2050 | CA | MET | A | 276 | 36.160 | 50.815 | 53.709 | 1.00 | 32.11 | A | C |
| ATOM | 2051 | CB | MET | A | 276 | 34.685 | 51.216 | 53.746 | 1.00 | 32.01 | A | C |
| ATOM | 2052 | CG | MET | A | 276 | 34.479 | 52.704 | 53.433 | 1.00 | 40.50 | A | C |
| ATOM | 2053 | SD | MET | A | 276 | 32.750 | 53.305 | 53.515 | 1.00 | 46.21 | A | S |
| ATOM | 2054 | CE | MET | A | 276 | 32.942 | 54.618 | 54.621 | 1.00 | 51.12 | A | C |
| ATOM | 2055 | C | MET | A | 276 | 36.898 | 51.572 | 54.787 | 1.00 | 30.47 | A | C |
| ATOM | 2056 | O | MET | A | 276 | 36.772 | 51.261 | 55.976 | 1.00 | 29.79 | A | O |
| ATOM | 2057 | N | VAL | A | 277 | 37.681 | 52.554 | 54.364 | 1.00 | 28.89 | A | N |
| ATOM | 2058 | CA | VAL | A | 277 | 38.408 | 53.394 | 55.287 | 1.00 | 27.83 | A | C |
| ATOM | 2059 | CB | VAL | A | 277 | 39.905 | 53.460 | 54.940 | 1.00 | 31.92 | A | C |
| ATOM | 2060 | CG1 | VAL | A | 277 | 40.614 | 54.399 | 55.896 | 1.00 | 27.69 | A | C |
| ATOM | 2061 | CG2 | VAL | A | 277 | 40.513 | 52.079 | 54.989 | 1.00 | 28.29 | A | C |
| ATOM | 2062 | C | VAL | A | 277 | 37.799 | 54.784 | 55.142 | 1.00 | 31.61 | A | C |
| ATOM | 2063 | O | VAL | A | 277 | 37.875 | 55.398 | 54.066 | 1.00 | 30.21 | A | O |
| ATOM | 2064 | N | ALA | A | 278 | 37.197 | 55.273 | 56.224 | 1.00 | 27.91 | A | N |
| ATOM | 2065 | CA | ALA | A | 278 | 36.557 | 56.573 | 56.216 | 1.00 | 31.01 | A | C |
| ATOM | 2066 | CB | ALA | A | 278 | 35.086 | 56.412 | 56.548 | 1.00 | 26.81 | A | C |
| ATOM | 2067 | C | ALA | A | 278 | 37.206 | 57.543 | 57.200 | 1.00 | 30.79 | A | C |
| ATOM | 2068 | O | ALA | A | 278 | 37.517 | 57.183 | 58.342 | 1.00 | 30.61 | A | O |
| ATOM | 2069 | N | LEU | A | 279 | 37.416 | 58.772 | 56.749 | 1.00 | 30.58 | A | N |
| ATOM | 2070 | CA | LEU | A | 279 | 37.996 | 59.796 | 57.604 | 1.00 | 31.94 | A | C |
| ATOM | 2071 | CB | LEU | A | 279 | 39.305 | 60.311 | 57.001 | 1.00 | 31.79 | A | C |
| ATOM | 2072 | CG | LEU | A | 279 | 39.950 | 61.452 | 57.785 | 1.00 | 34.78 | A | C |
| ATOM | 2073 | CD1 | LEU | A | 279 | 40.358 | 60.932 | 59.153 | 1.00 | 35.88 | A | C |
| ATOM | 2074 | CD2 | LEU | A | 279 | 41.170 | 61.993 | 57.035 | 1.00 | 33.69 | A | C |
| ATOM | 2075 | C | LEU | A | 279 | 37.005 | 60.943 | 57.788 | 1.00 | 30.91 | A | C |
| ATOM | 2076 | O | LEU | A | 279 | 36.735 | 61.700 | 56.849 | 1.00 | 34.28 | A | O |
| ATOM | 2077 | N | GLY | A | 280 | 36.456 | 61.062 | 58.997 | 1.00 | 31.34 | A | N |
| ATOM | 2078 | CA | GLY | A | 280 | 35.505 | 62.124 | 59.291 | 1.00 | 30.02 | A | C |
| ATOM | 2079 | C | GLY | A | 280 | 36.131 | 63.223 | 60.138 | 1.00 | 34.17 | A | C |
| ATOM | 2080 | O | GLY | A | 280 | 37.349 | 63.267 | 60.259 | 1.00 | 35.36 | A | O |
| ATOM | 2081 | N | GLU | A | 281 | 35.326 | 64.103 | 60.736 | 1.00 | 34.42 | A | N |
| ATOM | 2082 | CA | GLU | A | 281 | 35.891 | 65.197 | 61.550 | 1.00 | 39.01 | A | C |
| ATOM | 2083 | CB | GLU | A | 281 | 34.807 | 66.205 | 61.963 | 1.00 | 44.02 | A | C |
| ATOM | 2084 | CG | GLU | A | 281 | 34.296 | 67.088 | 60.817 | 1.00 | 48.95 | A | C |
| ATOM | 2085 | CD | GLU | A | 281 | 35.412 | 67.905 | 60.169 | 1.00 | 51.75 | A | C |
| ATOM | 2086 | OE1 | GLU | A | 281 | 35.734 | 67.664 | 58.981 | 1.00 | 49.15 | A | O |
| ATOM | 2087 | OE2 | GLU | A | 281 | 35.975 | 68.786 | 60.856 | 1.00 | 55.36 | A | O |
| ATOM | 2088 | C | GLU | A | 281 | 36.630 | 64.723 | 62.796 | 1.00 | 39.59 | A | C |
| ATOM | 2089 | O | GLU | A | 281 | 37.613 | 65.337 | 63.205 | 1.00 | 38.91 | A | O |
| ATOM | 2090 | N | GLU | A | 282 | 36.154 | 63.654 | 63.429 | 1.00 | 36.94 | A | N |
| ATOM | 2091 | CA | GLU | A | 282 | 36.870 | 63.167 | 64.589 | 1.00 | 36.46 | A | C |
| ATOM | 2092 | CB | GLU | A | 282 | 36.169 | 63.560 | 65.885 | 1.00 | 40.96 | A | C |
| ATOM | 2093 | CG | GLU | A | 282 | 34.805 | 62.989 | 66.104 | 1.00 | 45.06 | A | C |
| ATOM | 2094 | CD | GLU | A | 282 | 34.269 | 63.400 | 67.471 | 1.00 | 52.93 | A | C |
| ATOM | 2095 | OE1 | GLU | A | 282 | 34.665 | 62.774 | 68.485 | 1.00 | 48.53 | A | O |
| ATOM | 2096 | OE2 | GLU | A | 282 | 33.473 | 64.364 | 67.532 | 1.00 | 52.28 | A | O |
| ATOM | 2097 | C | GLU | A | 282 | 37.156 | 61.673 | 64.585 | 1.00 | 35.57 | A | C |
| ATOM | 2098 | O | GLU | A | 282 | 37.994 | 61.214 | 65.365 | 1.00 | 33.25 | A | O |
| ATOM | 2099 | N | ASP | A | 283 | 36.479 | 60.917 | 63.719 | 1.00 | 29.85 | A | N |
| ATOM | 2100 | CA | ASP | A | 283 | 36.718 | 59.475 | 63.642 | 1.00 | 30.34 | A | C |
| ATOM | 2101 | CB | ASP | A | 283 | 35.409 | 58.657 | 63.706 | 1.00 | 27.87 | A | C |
| ATOM | 2102 | CG | ASP | A | 283 | 34.686 | 58.768 | 65.040 | 1.00 | 26.04 | A | C |
| ATOM | 2103 | OD1 | ASP | A | 283 | 35.243 | 59.336 | 65.990 | 1.00 | 35.66 | A | O |
| ATOM | 2104 | OD2 | ASP | A | 283 | 33.551 | 58.261 | 65.147 | 1.00 | 29.89 | A | O |
| ATOM | 2105 | C | ASP | A | 283 | 37.405 | 59.053 | 62.345 | 1.00 | 30.81 | A | C |
| ATOM | 2106 | O | ASP | A | 283 | 37.184 | 59.643 | 61.278 | 1.00 | 29.62 | A | O |
| ATOM | 2107 | N | LEU | A | 284 | 38.232 | 58.018 | 62.455 | 1.00 | 27.08 | A | N |
| ATOM | 2108 | CA | LEU | A | 284 | 38.868 | 57.394 | 61.309 | 1.00 | 25.96 | A | C |
| ATOM | 2109 | CB | LEU | A | 284 | 40.383 | 57.445 | 61.418 | 1.00 | 28.33 | A | C |
| ATOM | 2110 | CG | LEU | A | 284 | 41.094 | 57.195 | 60.081 | 1.00 | 37.50 | A | C |
| ATOM | 2111 | CD1 | LEU | A | 284 | 42.603 | 57.381 | 60.262 | 1.00 | 41.04 | A | C |
| ATOM | 2112 | CD2 | LEU | A | 284 | 40.797 | 55.793 | 59.568 | 1.00 | 37.16 | A | C |
| ATOM | 2113 | C | LEU | A | 284 | 38.327 | 55.956 | 61.481 | 1.00 | 29.12 | A | C |
| ATOM | 2114 | O | LEU | A | 284 | 38.655 | 55.249 | 62.454 | 1.00 | 25.28 | A | O |
| ATOM | 2115 | N | SER | A | 285 | 37.460 | 55.533 | 60.566 | 1.00 | 23.31 | A | N |
| ATOM | 2116 | CA | SER | A | 285 | 36.835 | 54.219 | 60.692 | 1.00 | 23.15 | A | C |
| ATOM | 2117 | CB | SER | A | 285 | 35.320 | 54.396 | 60.733 | 1.00 | 25.57 | A | C |
| ATOM | 2118 | OG | SER | A | 285 | 34.999 | 55.449 | 61.630 | 1.00 | 22.77 | A | O |
| ATOM | 2119 | C | SER | A | 285 | 37.198 | 53.236 | 59.600 | 1.00 | 25.42 | A | C |
| ATOM | 2120 | O | SER | A | 285 | 37.289 | 53.601 | 58.432 | 1.00 | 26.19 | A | O |
| ATOM | 2121 | N | ILE | A | 286 | 37.370 | 51.973 | 59.988 | 1.00 | 24.91 | A | N |
| ATOM | 2122 | CA | ILE | A | 286 | 37.730 | 50.927 | 59.047 | 1.00 | 25.14 | A | C |
| ATOM | 2123 | CB | ILE | A | 286 | 39.136 | 50.356 | 59.366 | 1.00 | 25.19 | A | C |
| ATOM | 2124 | CG2 | ILE | A | 286 | 39.576 | 49.372 | 58.281 | 1.00 | 20.77 | A | C |
| ATOM | 2125 | CG1 | ILE | A | 286 | 40.137 | 51.514 | 59.490 | 1.00 | 29.27 | A | C |
| ATOM | 2126 | CD1 | ILE | A | 286 | 41.465 | 51.136 | 60.119 | 1.00 | 34.60 | A | C |
| ATOM | 2127 | C | ILE | A | 286 | 36.700 | 49.816 | 59.168 | 1.00 | 28.56 | A | C |
| ATOM | 2128 | O | ILE | A | 286 | 36.606 | 49.150 | 60.205 | 1.00 | 26.15 | A | O |
| ATOM | 2129 | N | LYS | A | 287 | 35.911 | 49.616 | 58.123 | 1.00 | 25.39 | A | N |
| ATOM | 2130 | CA | LYS | A | 287 | 34.939 | 48.545 | 58.175 | 1.00 | 27.25 | A | C |
| ATOM | 2131 | CB | LYS | A | 287 | 33.541 | 49.064 | 57.808 | 1.00 | 26.60 | A | C |
| ATOM | 2132 | CG | LYS | A | 287 | 32.626 | 47.949 | 57.340 | 1.00 | 29.04 | A | C |
| ATOM | 2133 | CD | LYS | A | 287 | 31.217 | 48.067 | 57.845 | 1.00 | 36.55 | A | C |
| ATOM | 2134 | CE | LYS | A | 287 | 30.501 | 49.289 | 57.333 | 1.00 | 42.82 | A | C |
| ATOM | 2135 | NZ | LYS | A | 287 | 29.052 | 49.179 | 57.695 | 1.00 | 42.30 | A | N |
| ATOM | 2136 | C | LYS | A | 287 | 35.345 | 47.389 | 57.247 | 1.00 | 27.30 | A | C |
| ATOM | 2137 | O | LYS | A | 287 | 35.698 | 47.603 | 56.093 | 1.00 | 26.80 | A | O |
| ATOM | 2138 | N | MET | A | 288 | 35.312 | 46.173 | 57.778 | 1.00 | 24.82 | A | N |
| ATOM | 2139 | CA | MET | A | 288 | 35.623 | 44.983 | 57.005 | 1.00 | 26.63 | A | C |
| ATOM | 2140 | CB | MET | A | 288 | 36.724 | 44.169 | 57.693 | 1.00 | 29.33 | A | C |
| ATOM | 2141 | CG | MET | A | 288 | 37.175 | 42.939 | 56.897 | 1.00 | 28.74 | A | C |
| ATOM | 2142 | SD | MET | A | 288 | 38.574 | 42.096 | 57.687 | 1.00 | 36.66 | A | S |
| ATOM | 2143 | CE | MET | A | 288 | 37.719 | 41.041 | 58.705 | 1.00 | 40.41 | A | C |
| ATOM | 2144 | C | MET | A | 288 | 34.347 | 44.151 | 56.879 | 1.00 | 28.40 | A | C |
| ATOM | 2145 | O | MET | A | 288 | 33.835 | 43.644 | 57.874 | 1.00 | 27.69 | A | O |
| ATOM | 2146 | N | SER | A | 289 | 33.855 | 44.012 | 55.648 | 1.00 | 25.75 | A | N |
| ATOM | 2147 | CA | SER | A | 289 | 32.631 | 43.273 | 55.339 | 1.00 | 29.44 | A | C |
| ATOM | 2148 | CB | SER | A | 289 | 31.701 | 44.121 | 54.458 | 1.00 | 29.61 | A | C |
| ATOM | 2149 | OG | SER | A | 289 | 31.457 | 45.382 | 55.041 | 1.00 | 32.18 | A | O |
| ATOM | 2150 | C | SER | A | 289 | 32.915 | 41.997 | 54.587 | 1.00 | 29.40 | A | C |
| ATOM | 2151 | O | SER | A | 289 | 33.585 | 42.036 | 53.554 | 1.00 | 29.47 | A | O |
| ATOM | 2152 | N | ASP | A | 290 | 32.400 | 40.874 | 55.089 | 1.00 | 29.55 | A | N |
| ATOM | 2153 | CA | ASP | A | 290 | 32.578 | 39.581 | 54.422 | 1.00 | 29.68 | A | C |
| ATOM | 2154 | CB | ASP | A | 290 | 33.414 | 38.624 | 55.274 | 1.00 | 28.84 | A | C |
| ATOM | 2155 | CG | ASP | A | 290 | 32.692 | 38.180 | 56.553 | 1.00 | 36.25 | A | C |
| ATOM | 2156 | OD1 | ASP | A | 290 | 31.595 | 37.572 | 56.461 | 1.00 | 32.48 | A | O |
| ATOM | 2157 | OD2 | ASP | A | 290 | 33.226 | 38.440 | 57.655 | 1.00 | 34.03 | A | O |
| ATOM | 2158 | C | ASP | A | 290 | 31.205 | 38.961 | 54.214 | 1.00 | 31.70 | A | C |
| ATOM | 2159 | O | ASP | A | 290 | 30.204 | 39.466 | 54.742 | 1.00 | 29.71 | A | O |
| ATOM | 2160 | N | ARG | A | 291 | 31.161 | 37.868 | 53.455 | 1.00 | 27.75 | A | N |
| ATOM | 2161 | CA | ARG | A | 291 | 29.926 | 37.152 | 53.201 | 1.00 | 31.22 | A | C |
| ATOM | 2162 | CB | ARG | A | 291 | 29.555 | 37.215 | 51.724 | 1.00 | 37.06 | A | C |
| ATOM | 2163 | CG | ARG | A | 291 | 29.145 | 38.605 | 51.305 | 1.00 | 47.22 | A | C |
| ATOM | 2164 | CD | ARG | A | 291 | 28.745 | 38.699 | 49.851 | 1.00 | 58.33 | A | C |
| ATOM | 2165 | NE | ARG | A | 291 | 28.435 | 40.083 | 49.490 | 1.00 | 69.58 | A | N |
| ATOM | 2166 | CZ | ARG | A | 291 | 29.289 | 41.104 | 49.596 | 1.00 | 77.30 | A | C |
| ATOM | 2167 | NH1 | ARG | A | 291 | 30.524 | 40.904 | 50.053 | 1.00 | 81.47 | A | N |
| ATOM | 2168 | NH2 | ARG | A | 291 | 28.905 | 42.334 | 49.253 | 1.00 | 76.73 | A | N |
| ATOM | 2169 | C | ARG | A | 291 | 30.175 | 35.727 | 53.630 | 1.00 | 34.74 | A | C |
| ATOM | 2170 | O | ARG | A | 291 | 29.821 | 34.781 | 52.930 | 1.00 | 38.75 | A | O |
| ATOM | 2171 | N | GLY | A | 292 | 30.775 | 35.592 | 54.811 | 1.00 | 33.61 | A | N |
| ATOM | 2172 | CA | GLY | A | 292 | 31.123 | 34.292 | 55.358 | 1.00 | 32.51 | A | C |
| ATOM | 2173 | C | GLY | A | 292 | 30.026 | 33.505 | 56.042 | 1.00 | 35.14 | A | C |
| ATOM | 2174 | O | GLY | A | 292 | 30.313 | 32.593 | 56.817 | 1.00 | 33.07 | A | O |
| ATOM | 2175 | N | GLY | A | 293 | 28.771 | 33.863 | 55.782 | 1.00 | 37.30 | A | N |
| ATOM | 2176 | CA | GLY | A | 293 | 27.667 | 33.134 | 56.383 | 1.00 | 36.77 | A | C |
| ATOM | 2177 | C | GLY | A | 293 | 27.043 | 33.750 | 57.622 | 1.00 | 36.42 | A | C |
| ATOM | 2178 | O | GLY | A | 293 | 25.881 | 33.478 | 57.924 | 1.00 | 35.16 | A | O |
| ATOM | 2179 | N | GLY | A | 294 | 27.807 | 34.560 | 58.349 | 1.00 | 30.75 | A | N |
| ATOM | 2180 | CA | GLY | A | 294 | 27.276 | 35.194 | 59.542 | 1.00 | 31.42 | A | C |
| ATOM | 2181 | C | GLY | A | 294 | 27.212 | 34.323 | 60.787 | 1.00 | 34.68 | A | C |
| ATOM | 2182 | O | GLY | A | 294 | 27.474 | 33.113 | 60.746 | 1.00 | 33.89 | A | O |
| ATOM | 2183 | N | VAL | A | 295 | 26.839 | 34.952 | 61.897 | 1.00 | 32.09 | A | N |
| ATOM | 2184 | CA | VAL | A | 295 | 26.736 | 34.293 | 63.198 | 1.00 | 31.09 | A | C |
| ATOM | 2185 | CB | VAL | A | 295 | 27.984 | 34.608 | 64.036 | 1.00 | 35.08 | A | C |
| ATOM | 2186 | CG1 | VAL | A | 295 | 27.794 | 34.179 | 65.480 | 1.00 | 32.46 | A | C |
| ATOM | 2187 | CG2 | VAL | A | 295 | 29.185 | 33.908 | 63.426 | 1.00 | 36.94 | A | C |
| ATOM | 2188 | C | VAL | A | 295 | 25.498 | 34.806 | 63.940 | 1.00 | 32.51 | A | C |
| ATOM | 2189 | O | VAL | A | 295 | 25.190 | 35.991 | 63.870 | 1.00 | 28.44 | A | O |
| ATOM | 2190 | N | PRO | A | 296 | 24.763 | 33.920 | 64.645 | 1.00 | 29.32 | A | N |
| ATOM | 2191 | CD | PRO | A | 296 | 25.140 | 32.566 | 65.075 | 1.00 | 27.66 | A | C |
| ATOM | 2192 | CA | PRO | A | 296 | 23.574 | 34.396 | 65.370 | 1.00 | 30.44 | A | C |
| ATOM | 2193 | CB | PRO | A | 296 | 23.050 | 33.151 | 66.082 | 1.00 | 27.08 | A | C |
| ATOM | 2194 | CG | PRO | A | 296 | 23.842 | 31.999 | 65.496 | 1.00 | 31.53 | A | C |
| ATOM | 2195 | C | PRO | A | 296 | 24.014 | 35.443 | 66.401 | 1.00 | 31.99 | A | C |
| ATOM | 2196 | O | PRO | A | 296 | 25.152 | 35.389 | 66.899 | 1.00 | 33.73 | A | O |
| ATOM | 2197 | N | LEU | A | 297 | 23.109 | 36.352 | 66.753 | 1.00 | 29.33 | A | N |
| ATOM | 2198 | CA | LEU | A | 297 | 23.427 | 37.399 | 67.718 | 1.00 | 32.96 | A | C |
| ATOM | 2199 | CB | LEU | A | 297 | 22.198 | 38.278 | 67.990 | 1.00 | 30.72 | A | C |
| ATOM | 2200 | CG | LEU | A | 297 | 22.457 | 39.508 | 68.885 | 1.00 | 38.24 | A | C |
| ATOM | 2201 | CD1 | LEU | A | 297 | 23.582 | 40.351 | 68.287 | 1.00 | 33.09 | A | C |
| ATOM | 2202 | CD2 | LEU | A | 297 | 21.181 | 40.352 | 69.011 | 1.00 | 34.80 | A | C |
| ATOM | 2203 | C | LEU | A | 297 | 23.948 | 36.866 | 69.039 | 1.00 | 32.90 | A | C |
| ATOM | 2204 | O | LEU | A | 297 | 24.911 | 37.414 | 69.594 | 1.00 | 31.14 | A | O |
| ATOM | 2205 | N | ARG | A | 298 | 23.322 | 35.806 | 69.548 | 1.00 | 33.34 | A | N |
| ATOM | 2206 | CA | ARG | A | 298 | 23.738 | 35.254 | 70.832 | 1.00 | 36.53 | A | C |
| ATOM | 2207 | CB | ARG | A | 298 | 22.791 | 34.140 | 71.305 | 1.00 | 43.86 | A | C |
| ATOM | 2208 | CG | ARG | A | 298 | 22.381 | 33.128 | 70.267 | 1.00 | 54.21 | A | C |
| ATOM | 2209 | CD | ARG | A | 298 | 21.596 | 31.988 | 70.925 | 1.00 | 57.26 | A | C |
| ATOM | 2210 | NE | ARG | A | 298 | 22.488 | 30.998 | 71.515 | 1.00 | 58.95 | A | N |
| ATOM | 2211 | CZ | ARG | A | 298 | 23.091 | 30.030 | 70.827 | 1.00 | 59.44 | A | C |
| ATOM | 2212 | NH1 | ARG | A | 298 | 22.896 | 29.903 | 69.518 | 1.00 | 57.48 | A | N |
| ATOM | 2213 | NH2 | ARG | A | 298 | 23.910 | 29.196 | 71.447 | 1.00 | 63.08 | A | N |
| ATOM | 2214 | C | ARG | A | 298 | 25.168 | 34.770 | 70.887 | 1.00 | 36.09 | A | C |
| ATOM | 2215 | O | ARG | A | 298 | 25.795 | 34.847 | 71.931 | 1.00 | 38.53 | A | O |
| ATOM | 2216 | N | LYS | A | 299 | 25.707 | 34.284 | 69.776 | 1.00 | 39.21 | A | N |
| ATOM | 2217 | CA | LYS | A | 299 | 27.094 | 33.820 | 69.806 | 1.00 | 39.89 | A | C |
| ATOM | 2218 | CB | LYS | A | 299 | 27.316 | 32.750 | 68.731 | 1.00 | 38.47 | A | C |
| ATOM | 2219 | CG | LYS | A | 299 | 26.427 | 31.532 | 68.979 | 1.00 | 47.71 | A | C |
| ATOM | 2220 | CD | LYS | A | 299 | 26.718 | 30.321 | 68.098 | 1.00 | 53.75 | A | C |
| ATOM | 2221 | CE | LYS | A | 299 | 25.732 | 29.189 | 68.473 | 1.00 | 61.23 | A | C |
| ATOM | 2222 | NZ | LYS | A | 299 | 25.797 | 27.966 | 67.606 | 1.00 | 63.40 | A | N |
| ATOM | 2223 | C | LYS | A | 299 | 28.092 | 34.972 | 69.656 | 1.00 | 42.20 | A | C |
| ATOM | 2224 | O | LYS | A | 299 | 29.264 | 34.836 | 70.005 | 1.00 | 41.85 | A | O |
| ATOM | 2225 | N | ILE | A | 300 | 27.629 | 36.115 | 69.153 | 1.00 | 45.50 | A | N |
| ATOM | 2226 | CA | ILE | A | 300 | 28.515 | 37.270 | 68.996 | 1.00 | 46.85 | A | C |
| ATOM | 2227 | CB | ILE | A | 300 | 27.783 | 38.466 | 68.344 | 1.00 | 49.29 | A | C |
| ATOM | 2228 | CG2 | ILE | A | 300 | 28.592 | 39.740 | 68.514 | 1.00 | 48.90 | A | C |
| ATOM | 2229 | CG1 | ILE | A | 300 | 27.583 | 38.195 | 66.858 | 1.00 | 45.55 | A | C |
| ATOM | 2230 | CD1 | ILE | A | 300 | 28.883 | 38.015 | 66.104 | 1.00 | 50.69 | A | C |
| ATOM | 2231 | C | ILE | A | 300 | 29.040 | 37.708 | 70.353 | 1.00 | 48.43 | A | C |
| ATOM | 2232 | O | ILE | A | 300 | 30.235 | 37.917 | 70.537 | 1.00 | 43.91 | A | O |
| ATOM | 2233 | N | GLU | A | 301 | 28.123 | 37.814 | 71.305 | 1.00 | 54.76 | A | N |
| ATOM | 2234 | CA | GLU | A | 301 | 28.434 | 38.246 | 72.662 | 1.00 | 59.49 | A | C |
| ATOM | 2235 | CB | GLU | A | 301 | 27.171 | 38.142 | 73.528 | 1.00 | 69.60 | A | C |
| ATOM | 2236 | CG | GLU | A | 301 | 25.834 | 38.290 | 72.762 | 1.00 | 78.13 | A | C |
| ATOM | 2237 | CD | GLU | A | 301 | 25.443 | 39.738 | 72.455 | 1.00 | 85.45 | A | C |
| ATOM | 2238 | OE1 | GLU | A | 301 | 26.252 | 40.468 | 71.846 | 1.00 | 90.67 | A | O |
| ATOM | 2239 | OE2 | GLU | A | 301 | 24.315 | 40.146 | 72.815 | 1.00 | 88.41 | A | O |
| ATOM | 2240 | C | GLU | A | 301 | 29.557 | 37.437 | 73.310 | 1.00 | 60.54 | A | C |
| ATOM | 2241 | O | GLU | A | 301 | 30.379 | 37.982 | 74.044 | 1.00 | 61.20 | A | O |
| ATOM | 2242 | N | ARG | A | 302 | 29.589 | 36.137 | 73.027 | 1.00 | 58.19 | A | N |
| ATOM | 2243 | CA | ARG | A | 302 | 30.586 | 35.230 | 73.597 | 1.00 | 55.84 | A | C |
| ATOM | 2244 | CB | ARG | A | 302 | 30.062 | 33.796 | 73.525 | 1.00 | 59.25 | A | C |
| ATOM | 2245 | CG | ARG | A | 302 | 28.630 | 33.659 | 74.011 | 1.00 | 66.79 | A | C |
| ATOM | 2246 | CD | ARG | A | 302 | 28.096 | 32.254 | 73.778 | 1.00 | 71.42 | A | C |
| ATOM | 2247 | NE | ARG | A | 302 | 28.805 | 31.269 | 74.586 | 1.00 | 77.31 | A | N |
| ATOM | 2248 | CZ | ARG | A | 302 | 28.753 | 31.215 | 75.915 | 1.00 | 79.41 | A | C |
| ATOM | 2249 | NH1 | ARG | A | 302 | 28.020 | 32.092 | 76.598 | 1.00 | 78.03 | A | N |
| ATOM | 2250 | NH2 | ARG | A | 302 | 29.440 | 30.282 | 76.562 | 1.00 | 79.26 | A | N |
| ATOM | 2251 | C | ARG | A | 302 | 31.950 | 35.311 | 72.915 | 1.00 | 51.42 | A | C |
| ATOM | 2252 | O | ARG | A | 302 | 32.972 | 34.924 | 73.486 | 1.00 | 48.03 | A | O |
| ATOM | 2253 | N | LEU | A | 303 | 31.963 | 35.811 | 71.687 | 1.00 | 47.82 | A | N |
| ATOM | 2254 | CA | LEU | A | 303 | 33.207 | 35.934 | 70.951 | 1.00 | 42.33 | A | C |
| ATOM | 2255 | CB | LEU | A | 303 | 32.936 | 36.442 | 69.528 | 1.00 | 41.92 | A | C |
| ATOM | 2256 | CG | LEU | A | 303 | 32.330 | 35.430 | 68.547 | 1.00 | 40.66 | A | C |
| ATOM | 2257 | CD1 | LEU | A | 303 | 32.121 | 36.085 | 67.175 | 1.00 | 44.63 | A | C |
| ATOM | 2258 | CD2 | LEU | A | 303 | 33.266 | 34.230 | 68.415 | 1.00 | 39.74 | A | C |
| ATOM | 2259 | C | LEU | A | 303 | 34.204 | 36.846 | 71.653 | 1.00 | 41.58 | A | C |
| ATOM | 2260 | O | LEU | A | 303 | 35.409 | 36.725 | 71.430 | 1.00 | 40.82 | A | O |
| ATOM | 2261 | N | PHE | A | 304 | 33.717 | 37.756 | 72.500 | 1.00 | 39.29 | A | N |
| ATOM | 2262 | CA | PHE | A | 304 | 34.626 | 38.654 | 73.213 | 1.00 | 39.70 | A | C |
| ATOM | 2263 | CB | PHE | A | 304 | 34.082 | 40.086 | 73.236 | 1.00 | 38.06 | A | C |
| ATOM | 2264 | CG | PHE | A | 304 | 34.190 | 40.793 | 71.908 | 1.00 | 35.88 | A | C |
| ATOM | 2265 | CD1 | PHE | A | 304 | 33.113 | 40.829 | 71.033 | 1.00 | 36.04 | A | C |
| ATOM | 2266 | CD2 | PHE | A | 304 | 35.384 | 41.384 | 71.523 | 1.00 | 34.49 | A | C |
| ATOM | 2267 | CE1 | PHE | A | 304 | 33.223 | 41.445 | 69.794 | 1.00 | 36.94 | A | C |
| ATOM | 2268 | CE2 | PHE | A | 304 | 35.508 | 42.004 | 70.282 | 1.00 | 35.38 | A | C |
| ATOM | 2269 | CZ | PHE | A | 304 | 34.421 | 42.033 | 69.417 | 1.00 | 33.47 | A | C |
| ATOM | 2270 | C | PHE | A | 304 | 34.950 | 38.218 | 74.638 | 1.00 | 44.73 | A | C |
| ATOM | 2271 | O | PHE | A | 304 | 35.742 | 38.870 | 75.330 | 1.00 | 43.13 | A | O |
| ATOM | 2272 | N | SER | A | 305 | 34.347 | 37.113 | 75.072 | 1.00 | 45.29 | A | N |
| ATOM | 2273 | CA | SER | A | 305 | 34.588 | 36.606 | 76.416 | 1.00 | 47.19 | A | C |
| ATOM | 2274 | CB | SER | A | 305 | 33.361 | 35.858 | 76.923 | 1.00 | 49.11 | A | C |
| ATOM | 2275 | OG | SER | A | 305 | 33.705 | 35.071 | 78.046 | 1.00 | 49.50 | A | O |
| ATOM | 2276 | C | SER | A | 305 | 35.796 | 35.677 | 76.465 | 1.00 | 48.06 | A | C |
| ATOM | 2277 | O | SER | A | 305 | 35.893 | 34.741 | 75.678 | 1.00 | 45.89 | A | O |
| ATOM | 2278 | N | TYR | A | 306 | 36.716 | 35.946 | 77.386 | 1.00 | 47.13 | A | N |
| ATOM | 2279 | CA | TYR | A | 306 | 37.902 | 35.115 | 77.541 | 1.00 | 49.94 | A | C |
| ATOM | 2280 | CB | TYR | A | 306 | 38.830 | 35.678 | 78.620 | 1.00 | 46.12 | A | C |
| ATOM | 2281 | CG | TYR | A | 306 | 39.352 | 37.054 | 78.326 | 1.00 | 43.07 | A | C |
| ATOM | 2282 | CD1 | TYR | A | 306 | 38.755 | 38.185 | 78.893 | 1.00 | 42.03 | A | C |
| ATOM | 2283 | CE1 | TYR | A | 306 | 39.249 | 39.477 | 78.622 | 1.00 | 39.67 | A | C |
| ATOM | 2284 | CD2 | TYR | A | 306 | 40.447 | 37.232 | 77.478 | 1.00 | 40.09 | A | C |
| ATOM | 2285 | CE2 | TYR | A | 306 | 40.946 | 38.509 | 77.199 | 1.00 | 39.61 | A | C |
| ATOM | 2286 | CZ | TYR | A | 306 | 40.346 | 39.624 | 77.773 | 1.00 | 38.99 | A | C |
| ATOM | 2287 | OH | TYR | A | 306 | 40.854 | 40.876 | 77.508 | 1.00 | 41.07 | A | O |
| ATOM | 2288 | C | TYR | A | 306 | 37.509 | 33.706 | 77.949 | 1.00 | 53.51 | A | C |
| ATOM | 2289 | O | TYR | A | 306 | 38.025 | 32.726 | 77.422 | 1.00 | 53.74 | A | O |
| ATOM | 2290 | N | MET | A | 307 | 36.592 | 33.617 | 78.904 | 1.00 | 57.63 | A | N |
| ATOM | 2291 | CA | MET | A | 307 | 36.145 | 32.336 | 79.406 | 1.00 | 63.54 | A | C |
| ATOM | 2292 | CB | MET | A | 307 | 35.206 | 32.547 | 80.591 | 1.00 | 66.41 | A | C |
| ATOM | 2293 | CG | MET | A | 307 | 34.966 | 31.293 | 81.410 | 1.00 | 73.10 | A | C |
| ATOM | 2294 | SD | MET | A | 307 | 34.264 | 31.692 | 83.016 | 1.00 | 80.43 | A | S |
| ATOM | 2295 | CE | MET | A | 307 | 32.505 | 31.709 | 82.625 | 1.00 | 78.32 | A | C |
| ATOM | 2296 | C | MET | A | 307 | 35.452 | 31.512 | 78.329 | 1.00 | 66.99 | A | C |
| ATOM | 2297 | O | MET | A | 307 | 35.904 | 30.415 | 77.990 | 1.00 | 65.60 | A | O |
| ATOM | 2298 | N | TYR | A | 308 | 34.364 | 32.055 | 77.789 | 1.00 | 69.30 | A | N |
| ATOM | 2299 | CA | TYR | A | 308 | 33.579 | 31.384 | 76.757 | 1.00 | 73.01 | A | C |
| ATOM | 2300 | CB | TYR | A | 308 | 32.324 | 32.203 | 76.452 | 1.00 | 74.38 | A | C |
| ATOM | 2301 | CG | TYR | A | 308 | 31.420 | 32.418 | 77.650 | 1.00 | 77.49 | A | C |
| ATOM | 2302 | CD1 | TYR | A | 308 | 30.501 | 33.470 | 77.672 | 1.00 | 79.33 | A | C |
| ATOM | 2303 | CE1 | TYR | A | 308 | 29.656 | 33.677 | 78.765 | 1.00 | 79.92 | A | C |
| ATOM | 2304 | CD2 | TYR | A | 308 | 31.472 | 31.569 | 78.758 | 1.00 | 78.05 | A | C |
| ATOM | 2305 | CE2 | TYR | A | 308 | 30.632 | 31.764 | 79.857 | 1.00 | 79.48 | A | C |
| ATOM | 2306 | CZ | TYR | A | 308 | 29.727 | 32.822 | 79.855 | 1.00 | 81.72 | A | C |
| ATOM | 2307 | OH | TYR | A | 308 | 28.906 | 33.032 | 80.946 | 1.00 | 82.39 | A | O |
| ATOM | 2308 | C | TYR | A | 308 | 34.335 | 31.103 | 75.457 | 1.00 | 74.19 | A | C |
| ATOM | 2309 | O | TYR | A | 308 | 33.820 | 30.423 | 74.574 | 1.00 | 75.07 | A | O |
| ATOM | 2310 | N | SER | A | 309 | 35.551 | 31.621 | 75.334 | 1.00 | 75.87 | A | N |
| ATOM | 2311 | CA | SER | A | 309 | 36.339 | 31.387 | 74.129 | 1.00 | 80.83 | A | C |
| ATOM | 2312 | CB | SER | A | 309 | 36.720 | 32.720 | 73.475 | 1.00 | 81.64 | A | C |
| ATOM | 2313 | OG | SER | A | 309 | 37.648 | 33.445 | 74.267 | 1.00 | 81.86 | A | O |
| ATOM | 2314 | C | SER | A | 309 | 37.601 | 30.597 | 74.473 | 1.00 | 83.93 | A | C |
| ATOM | 2315 | O | SER | A | 309 | 38.641 | 30.751 | 73.830 | 1.00 | 82.46 | A | O |
| ATOM | 2316 | N | THR | A | 310 | 37.495 | 29.748 | 75.492 | 1.00 | 89.67 | A | N |
| ATOM | 2317 | CA | THR | A | 310 | 38.619 | 28.932 | 75.945 | 1.00 | 96.41 | A | C |
| ATOM | 2318 | CB | THR | A | 310 | 38.980 | 29.255 | 77.421 | 1.00 | 96.62 | A | C |
| ATOM | 2319 | OG1 | THR | A | 310 | 39.410 | 30.618 | 77.520 | 1.00 | 96.59 | A | O |
| ATOM | 2320 | CG2 | THR | A | 310 | 40.098 | 28.346 | 77.918 | 1.00 | 96.87 | A | C |
| ATOM | 2321 | C | THR | A | 310 | 38.344 | 27.433 | 75.823 | 1.00 | 100.67 | A | C |
| ATOM | 2322 | O | THR | A | 310 | 37.207 | 26.977 | 75.970 | 1.00 | 101.71 | A | O |
| ATOM | 2323 | N | ALA | A | 311 | 39.404 | 26.676 | 75.559 | 1.00 | 105.91 | A | N |
| ATOM | 2324 | CA | ALA | A | 311 | 39.314 | 25.228 | 75.411 | 1.00 | 110.52 | A | C |
| ATOM | 2325 | CB | ALA | A | 311 | 38.568 | 24.891 | 74.118 | 1.00 | 110.51 | A | C |
| ATOM | 2326 | C | ALA | A | 311 | 40.731 | 24.639 | 75.387 | 1.00 | 113.35 | A | C |
| ATOM | 2327 | O | ALA | A | 311 | 41.714 | 25.378 | 75.454 | 1.00 | 113.59 | A | O |
| ATOM | 2328 | N | PRO | A | 312 | 40.857 | 23.300 | 75.302 | 1.00 | 115.84 | A | N |
| ATOM | 2329 | CD | PRO | A | 312 | 42.163 | 22.697 | 74.983 | 1.00 | 116.52 | A | C |
| ATOM | 2330 | CA | PRO | A | 312 | 39.796 | 22.285 | 75.229 | 1.00 | 116.95 | A | C |
| ATOM | 2331 | CB | PRO | A | 312 | 40.569 | 21.000 | 74.930 | 1.00 | 117.45 | A | C |
| ATOM | 2332 | CG | PRO | A | 312 | 41.761 | 21.495 | 74.166 | 1.00 | 117.38 | A | C |
| ATOM | 2333 | C | PRO | A | 312 | 38.983 | 22.182 | 76.519 | 1.00 | 117.01 | A | C |
| ATOM | 2334 | O | PRO | A | 312 | 37.781 | 21.917 | 76.487 | 1.00 | 116.78 | A | O |
| ATOM | 2335 | N | GLY | A | 327 | 40.952 | 31.495 | 70.265 | 1.00 | 83.86 | A | N |
| ATOM | 2336 | CA | GLY | A | 327 | 41.886 | 32.220 | 71.107 | 1.00 | 83.28 | A | C |
| ATOM | 2337 | C | GLY | A | 327 | 41.338 | 33.562 | 71.559 | 1.00 | 82.39 | A | C |
| ATOM | 2338 | O | GLY | A | 327 | 40.136 | 33.815 | 71.440 | 1.00 | 83.19 | A | O |
| ATOM | 2339 | N | TYR | A | 328 | 42.214 | 34.422 | 72.078 | 1.00 | 79.65 | A | N |
| ATOM | 2340 | CA | TYR | A | 328 | 41.804 | 35.747 | 72.546 | 1.00 | 76.39 | A | C |
| ATOM | 2341 | CB | TYR | A | 328 | 42.513 | 36.099 | 73.854 | 1.00 | 80.33 | A | C |
| ATOM | 2342 | CG | TYR | A | 328 | 42.232 | 35.150 | 74.992 | 1.00 | 85.63 | A | C |
| ATOM | 2343 | CD1 | TYR | A | 328 | 41.015 | 34.468 | 75.077 | 1.00 | 88.11 | A | C |
| ATOM | 2344 | CE1 | TYR | A | 328 | 40.734 | 33.623 | 76.151 | 1.00 | 90.15 | A | C |
| ATOM | 2345 | CD2 | TYR | A | 328 | 43.167 | 34.964 | 76.011 | 1.00 | 87.62 | A | C |
| ATOM | 2346 | CE2 | TYR | A | 328 | 42.897 | 34.126 | 77.091 | 1.00 | 89.60 | A | C |
| ATOM | 2347 | CZ | TYR | A | 328 | 41.680 | 33.458 | 77.154 | 1.00 | 90.39 | A | C |
| ATOM | 2348 | OH | TYR | A | 328 | 41.411 | 32.627 | 78.217 | 1.00 | 91.85 | A | O |
| ATOM | 2349 | C | TYR | A | 328 | 42.078 | 36.851 | 71.523 | 1.00 | 70.83 | A | C |
| ATOM | 2350 | O | TYR | A | 328 | 42.151 | 38.033 | 71.880 | 1.00 | 67.85 | A | O |
| ATOM | 2351 | N | GLY | A | 329 | 42.230 | 36.452 | 70.258 | 1.00 | 63.33 | A | N |
| ATOM | 2352 | CA | GLY | A | 329 | 42.489 | 37.402 | 69.191 | 1.00 | 53.28 | A | C |
| ATOM | 2353 | C | GLY | A | 329 | 41.519 | 38.572 | 69.237 | 1.00 | 47.43 | A | C |
| ATOM | 2354 | O | GLY | A | 329 | 41.936 | 39.725 | 69.267 | 1.00 | 44.83 | A | O |
| ATOM | 2355 | N | LEU | A | 330 | 40.225 | 38.278 | 69.271 | 1.00 | 41.96 | A | N |
| ATOM | 2356 | CA | LEU | A | 330 | 39.226 | 39.328 | 69.306 | 1.00 | 40.57 | A | C |
| ATOM | 2357 | CB | LEU | A | 330 | 37.825 | 38.757 | 69.069 | 1.00 | 38.86 | A | C |
| ATOM | 2358 | CG | LEU | A | 330 | 37.411 | 38.632 | 67.599 | 1.00 | 44.08 | A | C |
| ATOM | 2359 | CD1 | LEU | A | 330 | 36.033 | 37.996 | 67.508 | 1.00 | 39.13 | A | C |
| ATOM | 2360 | CD2 | LEU | A | 330 | 37.410 | 40.010 | 66.947 | 1.00 | 39.50 | A | C |
| ATOM | 2361 | C | LEU | A | 330 | 39.218 | 40.200 | 70.561 | 1.00 | 38.33 | A | C |
| ATOM | 2362 | O | LEU | A | 330 | 39.349 | 41.414 | 70.450 | 1.00 | 35.70 | A | O |
| ATOM | 2363 | N | PRO | A | 331 | 39.077 | 39.605 | 71.764 | 1.00 | 37.84 | A | N |
| ATOM | 2364 | CD | PRO | A | 331 | 38.808 | 38.205 | 72.140 | 1.00 | 37.51 | A | C |
| ATOM | 2365 | CA | PRO | A | 331 | 39.067 | 40.483 | 72.941 | 1.00 | 39.14 | A | C |
| ATOM | 2366 | CB | PRO | A | 331 | 38.766 | 39.521 | 74.106 | 1.00 | 40.72 | A | C |
| ATOM | 2367 | CG | PRO | A | 331 | 39.193 | 38.179 | 73.600 | 1.00 | 36.96 | A | C |
| ATOM | 2368 | C | PRO | A | 331 | 40.341 | 41.305 | 73.122 | 1.00 | 40.41 | A | C |
| ATOM | 2369 | O | PRO | A | 331 | 40.289 | 42.469 | 73.544 | 1.00 | 39.22 | A | O |
| ATOM | 2370 | N | ILE | A | 332 | 41.487 | 40.725 | 72.788 | 1.00 | 37.69 | A | N |
| ATOM | 2371 | CA | ILE | A | 332 | 42.712 | 41.489 | 72.918 | 1.00 | 34.68 | A | C |
| ATOM | 2372 | CB | ILE | A | 332 | 43.964 | 40.592 | 72.880 | 1.00 | 36.31 | A | C |
| ATOM | 2373 | CG2 | ILE | A | 332 | 45.227 | 41.439 | 72.650 | 1.00 | 28.75 | A | C |
| ATOM | 2374 | CG1 | ILE | A | 332 | 44.070 | 39.822 | 74.201 | 1.00 | 36.80 | A | C |
| ATOM | 2375 | CD1 | ILE | A | 332 | 45.317 | 38.951 | 74.303 | 1.00 | 39.29 | A | C |
| ATOM | 2376 | C | ILE | A | 332 | 42.781 | 42.522 | 71.801 | 1.00 | 36.50 | A | C |
| ATOM | 2377 | O | ILE | A | 332 | 43.227 | 43.635 | 72.028 | 1.00 | 36.38 | A | O |
| ATOM | 2378 | N | SER | A | 333 | 42.345 | 42.170 | 70.596 | 1.00 | 33.66 | A | N |
| ATOM | 2379 | CA | SER | A | 333 | 42.371 | 43.146 | 69.512 | 1.00 | 32.37 | A | C |
| ATOM | 2380 | CB | SER | A | 333 | 41.844 | 42.530 | 68.208 | 1.00 | 35.03 | A | C |
| ATOM | 2381 | OG | SER | A | 333 | 42.827 | 41.695 | 67.608 | 1.00 | 34.93 | A | O |
| ATOM | 2382 | C | SER | A | 333 | 41.531 | 44.372 | 69.925 | 1.00 | 31.48 | A | C |
| ATOM | 2383 | O | SER | A | 333 | 41.930 | 45.507 | 69.688 | 1.00 | 30.90 | A | O |
| ATOM | 2384 | N | ARG | A | 334 | 40.392 | 44.133 | 70.577 | 1.00 | 28.24 | A | N |
| ATOM | 2385 | CA | ARG | A | 334 | 39.533 | 45.213 | 71.030 | 1.00 | 29.94 | A | C |
| ATOM | 2386 | CB | ARG | A | 334 | 38.276 | 44.660 | 71.682 | 1.00 | 32.37 | A | C |
| ATOM | 2387 | CG | ARG | A | 334 | 37.248 | 45.724 | 71.993 | 1.00 | 33.60 | A | C |
| ATOM | 2388 | CD | ARG | A | 334 | 36.433 | 45.269 | 73.168 | 1.00 | 40.04 | A | C |
| ATOM | 2389 | NE | ARG | A | 334 | 35.082 | 44.892 | 72.798 | 1.00 | 41.34 | A | N |
| ATOM | 2390 | CZ | ARG | A | 334 | 34.287 | 44.133 | 73.549 | 1.00 | 43.94 | A | C |
| ATOM | 2391 | NH1 | ARG | A | 334 | 34.709 | 43.649 | 74.721 | 1.00 | 39.19 | A | N |
| ATOM | 2392 | NH2 | ARG | A | 334 | 33.051 | 43.881 | 73.142 | 1.00 | 38.60 | A | N |
| ATOM | 2393 | C | ARG | A | 334 | 40.260 | 46.108 | 72.030 | 1.00 | 30.41 | A | C |
| ATOM | 2394 | O | ARG | A | 334 | 40.079 | 47.326 | 72.035 | 1.00 | 29.07 | A | O |
| ATOM | 2395 | N | LEU | A | 335 | 41.077 | 45.506 | 72.888 | 1.00 | 32.30 | A | N |
| ATOM | 2396 | CA | LEU | A | 335 | 41.830 | 46.283 | 73.863 | 1.00 | 29.75 | A | C |
| ATOM | 2397 | CB | LEU | A | 335 | 42.589 | 45.366 | 74.830 | 1.00 | 31.55 | A | C |
| ATOM | 2398 | CG | LEU | A | 335 | 41.715 | 44.495 | 75.740 | 1.00 | 35.84 | A | C |
| ATOM | 2399 | CD1 | LEU | A | 335 | 42.618 | 43.682 | 76.655 | 1.00 | 33.54 | A | C |
| ATOM | 2400 | CD2 | LEU | A | 335 | 40.759 | 45.356 | 76.564 | 1.00 | 31.98 | A | C |
| ATOM | 2401 | C | LEU | A | 335 | 42.805 | 47.193 | 73.131 | 1.00 | 27.16 | A | C |
| ATOM | 2402 | O | LEU | A | 335 | 43.004 | 48.323 | 73.543 | 1.00 | 29.95 | A | O |
| ATOM | 2403 | N | TYR | A | 336 | 43.430 | 46.717 | 72.055 | 1.00 | 27.01 | A | N |
| ATOM | 2404 | CA | TYR | A | 336 | 44.342 | 47.596 | 71.323 | 1.00 | 28.98 | A | C |
| ATOM | 2405 | CB | TYR | A | 336 | 45.040 | 46.875 | 70.169 | 1.00 | 35.07 | A | C |
| ATOM | 2406 | CG | TYR | A | 336 | 46.279 | 46.131 | 70.583 | 1.00 | 43.71 | A | C |
| ATOM | 2407 | CD1 | TYR | A | 336 | 46.221 | 44.778 | 70.902 | 1.00 | 42.86 | A | C |
| ATOM | 2408 | CE1 | TYR | A | 336 | 47.343 | 44.099 | 71.352 | 1.00 | 44.19 | A | C |
| ATOM | 2409 | CD2 | TYR | A | 336 | 47.502 | 46.795 | 70.721 | 1.00 | 42.63 | A | C |
| ATOM | 2410 | CE2 | TYR | A | 336 | 48.634 | 46.126 | 71.175 | 1.00 | 43.11 | A | C |
| ATOM | 2411 | CZ | TYR | A | 336 | 48.542 | 44.775 | 71.491 | 1.00 | 46.03 | A | C |
| ATOM | 2412 | OH | TYR | A | 336 | 49.631 | 44.102 | 71.988 | 1.00 | 45.39 | A | O |
| ATOM | 2413 | C | TYR | A | 336 | 43.577 | 48.788 | 70.740 | 1.00 | 30.89 | A | C |
| ATOM | 2414 | O | TYR | A | 336 | 44.118 | 49.891 | 70.623 | 1.00 | 31.76 | A | O |
| ATOM | 2415 | N | ALA | A | 337 | 42.312 | 48.573 | 70.379 | 1.00 | 31.35 | A | N |
| ATOM | 2416 | CA | ALA | A | 337 | 41.532 | 49.665 | 69.808 | 1.00 | 29.12 | A | C |
| ATOM | 2417 | CB | ALA | A | 337 | 40.246 | 49.132 | 69.182 | 1.00 | 27.21 | A | C |
| ATOM | 2418 | C | ALA | A | 337 | 41.210 | 50.667 | 70.909 | 1.00 | 28.39 | A | C |
| ATOM | 2419 | O | ALA | A | 337 | 41.383 | 51.873 | 70.734 | 1.00 | 27.28 | A | O |
| ATOM | 2420 | N | LYS | A | 338 | 40.767 | 50.149 | 72.053 | 1.00 | 29.44 | A | N |
| ATOM | 2421 | CA | LYS | A | 338 | 40.415 | 50.986 | 73.198 | 1.00 | 32.70 | A | C |
| ATOM | 2422 | CB | LYS | A | 338 | 39.761 | 50.140 | 74.292 | 1.00 | 32.31 | A | C |
| ATOM | 2423 | CG | LYS | A | 338 | 38.318 | 49.706 | 74.019 | 1.00 | 33.89 | A | C |
| ATOM | 2424 | CD | LYS | A | 338 | 37.820 | 48.830 | 75.167 | 1.00 | 32.87 | A | C |
| ATOM | 2425 | CE | LYS | A | 338 | 36.409 | 48.301 | 74.924 | 1.00 | 39.11 | A | C |
| ATOM | 2426 | NZ | LYS | A | 338 | 35.469 | 49.419 | 74.657 | 1.00 | 45.00 | A | N |
| ATOM | 2427 | C | LYS | A | 338 | 41.601 | 51.742 | 73.816 | 1.00 | 33.82 | A | C |
| ATOM | 2428 | O | LYS | A | 338 | 41.415 | 52.800 | 74.408 | 1.00 | 31.93 | A | O |
| ATOM | 2429 | N | TYR | A | 339 | 42.808 | 51.207 | 73.669 | 1.00 | 35.24 | A | N |
| ATOM | 2430 | CA | TYR | A | 339 | 44.006 | 51.817 | 74.259 | 1.00 | 36.24 | A | C |
| ATOM | 2431 | CB | TYR | A | 339 | 45.259 | 51.051 | 73.802 | 1.00 | 32.43 | A | C |
| ATOM | 2432 | CG | TYR | A | 339 | 46.574 | 51.589 | 74.335 | 1.00 | 35.61 | A | C |
| ATOM | 2433 | CD1 | TYR | A | 339 | 46.712 | 51.978 | 75.673 | 1.00 | 35.17 | A | C |
| ATOM | 2434 | CE1 | TYR | A | 339 | 47.936 | 52.482 | 76.159 | 1.00 | 36.86 | A | C |
| ATOM | 2435 | CD2 | TYR | A | 339 | 47.684 | 51.708 | 73.495 | 1.00 | 35.12 | A | C |
| ATOM | 2436 | CE2 | TYR | A | 339 | 48.907 | 52.205 | 73.966 | 1.00 | 39.36 | A | C |
| ATOM | 2437 | CZ | TYR | A | 339 | 49.025 | 52.591 | 75.297 | 1.00 | 40.33 | A | C |
| ATOM | 2438 | OH | TYR | A | 339 | 50.235 | 53.079 | 75.739 | 1.00 | 40.50 | A | O |
| ATOM | 2439 | C | TYR | A | 339 | 44.148 | 53.314 | 73.982 | 1.00 | 36.09 | A | C |
| ATOM | 2440 | O | TYR | A | 339 | 44.510 | 54.079 | 74.875 | 1.00 | 37.74 | A | O |
| ATOM | 2441 | N | PHE | A | 340 | 43.870 | 53.735 | 72.754 | 1.00 | 35.99 | A | N |
| ATOM | 2442 | CA | PHE | A | 340 | 43.940 | 55.155 | 72.417 | 1.00 | 34.02 | A | C |
| ATOM | 2443 | CB | PHE | A | 340 | 44.833 | 55.374 | 71.189 | 1.00 | 36.03 | A | C |
| ATOM | 2444 | CG | PHE | A | 340 | 46.307 | 55.381 | 71.511 | 1.00 | 41.39 | A | C |
| ATOM | 2445 | CD1 | PHE | A | 340 | 46.846 | 56.378 | 72.327 | 1.00 | 46.31 | A | C |
| ATOM | 2446 | CD2 | PHE | A | 340 | 47.151 | 54.382 | 71.031 | 1.00 | 43.18 | A | C |
| ATOM | 2447 | CE1 | PHE | A | 340 | 48.213 | 56.379 | 72.665 | 1.00 | 45.15 | A | C |
| ATOM | 2448 | CE2 | PHE | A | 340 | 48.520 | 54.372 | 71.360 | 1.00 | 45.86 | A | C |
| ATOM | 2449 | CZ | PHE | A | 340 | 49.047 | 55.374 | 72.180 | 1.00 | 47.04 | A | C |
| ATOM | 2450 | C | PHE | A | 340 | 42.546 | 55.763 | 72.200 | 1.00 | 33.16 | A | C |
| ATOM | 2451 | O | PHE | A | 340 | 42.351 | 56.608 | 71.327 | 1.00 | 33.20 | A | O |
| ATOM | 2452 | N | GLN | A | 341 | 41.582 | 55.319 | 73.006 | 1.00 | 31.73 | A | N |
| ATOM | 2453 | CA | GLN | A | 341 | 40.211 | 55.828 | 72.937 | 1.00 | 35.07 | A | C |
| ATOM | 2454 | CB | GLN | A | 341 | 40.211 | 57.371 | 72.959 | 1.00 | 35.28 | A | C |
| ATOM | 2455 | CG | GLN | A | 341 | 41.039 | 58.035 | 74.079 | 1.00 | 39.52 | A | C |
| ATOM | 2456 | CD | GLN | A | 341 | 40.583 | 57.663 | 75.486 | 1.00 | 44.10 | A | C |
| ATOM | 2457 | OE1 | GLN | A | 341 | 39.388 | 57.720 | 75.809 | 1.00 | 45.90 | A | O |
| ATOM | 2458 | NE2 | GLN | A | 341 | 41.541 | 57.294 | 76.342 | 1.00 | 45.81 | A | N |
| ATOM | 2459 | C | GLN | A | 341 | 39.417 | 55.352 | 71.707 | 1.00 | 33.34 | A | C |
| ATOM | 2460 | O | GLN | A | 341 | 38.501 | 56.034 | 71.260 | 1.00 | 31.04 | A | O |
| ATOM | 2461 | N | GLY | A | 342 | 39.764 | 54.195 | 71.160 | 1.00 | 32.14 | A | N |
| ATOM | 2462 | CA | GLY | A | 342 | 39.048 | 53.684 | 70.011 | 1.00 | 26.16 | A | C |
| ATOM | 2463 | C | GLY | A | 342 | 38.139 | 52.554 | 70.440 | 1.00 | 28.52 | A | C |
| ATOM | 2464 | O | GLY | A | 342 | 37.841 | 52.422 | 71.621 | 1.00 | 29.16 | A | O |
| ATOM | 2465 | N | ASP | A | 343 | 37.675 | 51.744 | 69.493 | 1.00 | 29.69 | A | N |
| ATOM | 2466 | CA | ASP | A | 343 | 36.815 | 50.615 | 69.834 | 1.00 | 26.08 | A | C |
| ATOM | 2467 | CB | ASP | A | 343 | 35.377 | 51.075 | 70.108 | 1.00 | 25.48 | A | C |
| ATOM | 2468 | CG | ASP | A | 343 | 34.576 | 50.067 | 70.944 | 1.00 | 26.97 | A | C |
| ATOM | 2469 | OD1 | ASP | A | 343 | 35.120 | 49.015 | 71.331 | 1.00 | 31.55 | A | O |
| ATOM | 2470 | OD2 | ASP | A | 343 | 33.386 | 50.315 | 71.208 | 1.00 | 30.66 | A | O |
| ATOM | 2471 | C | ASP | A | 343 | 36.846 | 49.628 | 68.679 | 1.00 | 25.37 | A | C |
| ATOM | 2472 | O | ASP | A | 343 | 37.364 | 49.941 | 67.607 | 1.00 | 23.98 | A | O |
| ATOM | 2473 | N | LEU | A | 344 | 36.367 | 48.410 | 68.920 | 1.00 | 26.44 | A | N |
| ATOM | 2474 | CA | LEU | A | 344 | 36.309 | 47.404 | 67.863 | 1.00 | 23.06 | A | C |
| ATOM | 2475 | CB | LEU | A | 344 | 37.455 | 46.407 | 67.989 | 1.00 | 30.58 | A | C |
| ATOM | 2476 | CG | LEU | A | 344 | 37.689 | 45.505 | 66.768 | 1.00 | 32.33 | A | C |
| ATOM | 2477 | CD1 | LEU | A | 344 | 39.108 | 44.983 | 66.781 | 1.00 | 31.07 | A | C |
| ATOM | 2478 | CD2 | LEU | A | 344 | 36.674 | 44.368 | 66.762 | 1.00 | 34.11 | A | C |
| ATOM | 2479 | C | LEU | A | 344 | 34.969 | 46.720 | 68.060 | 1.00 | 26.61 | A | C |
| ATOM | 2480 | O | LEU | A | 344 | 34.694 | 46.212 | 69.145 | 1.00 | 25.46 | A | O |
| ATOM | 2481 | N | GLN | A | 345 | 34.117 | 46.730 | 67.035 | 1.00 | 26.30 | A | N |
| ATOM | 2482 | CA | GLN | A | 345 | 32.795 | 46.126 | 67.172 | 1.00 | 24.20 | A | C |
| ATOM | 2483 | CB | GLN | A | 345 | 31.746 | 47.235 | 67.281 | 1.00 | 25.78 | A | C |
| ATOM | 2484 | CG | GLN | A | 345 | 32.062 | 48.294 | 68.346 | 1.00 | 26.23 | A | C |
| ATOM | 2485 | CD | GLN | A | 345 | 31.047 | 49.447 | 68.364 | 1.00 | 33.29 | A | C |
| ATOM | 2486 | OE1 | GLN | A | 345 | 30.189 | 49.572 | 67.475 | 1.00 | 27.53 | A | O |
| ATOM | 2487 | NE2 | GLN | A | 345 | 31.153 | 50.297 | 69.373 | 1.00 | 29.71 | A | N |
| ATOM | 2488 | C | GLN | A | 345 | 32.414 | 45.179 | 66.021 | 1.00 | 27.67 | A | C |
| ATOM | 2489 | O | GLN | A | 345 | 32.895 | 45.319 | 64.898 | 1.00 | 27.98 | A | O |
| ATOM | 2490 | N | LEU | A | 346 | 31.519 | 44.237 | 66.307 | 1.00 | 23.71 | A | N |
| ATOM | 2491 | CA | LEU | A | 346 | 31.062 | 43.273 | 65.311 | 1.00 | 27.24 | A | C |
| ATOM | 2492 | CB | LEU | A | 346 | 31.473 | 41.844 | 65.688 | 1.00 | 26.47 | A | C |
| ATOM | 2493 | CG | LEU | A | 346 | 32.949 | 41.522 | 65.871 | 1.00 | 35.62 | A | C |
| ATOM | 2494 | CD1 | LEU | A | 346 | 33.126 | 40.033 | 66.213 | 1.00 | 33.04 | A | C |
| ATOM | 2495 | CD2 | LEU | A | 346 | 33.665 | 41.874 | 64.593 | 1.00 | 28.35 | A | C |
| ATOM | 2496 | C | LEU | A | 346 | 29.560 | 43.239 | 65.222 | 1.00 | 28.01 | A | C |
| ATOM | 2497 | O | LEU | A | 346 | 28.887 | 43.379 | 66.229 | 1.00 | 26.83 | A | O |
| ATOM | 2498 | N | PHE | A | 347 | 29.047 | 43.045 | 64.013 | 1.00 | 23.53 | A | N |
| ATOM | 2499 | CA | PHE | A | 347 | 27.620 | 42.861 | 63.812 | 1.00 | 27.89 | A | C |
| ATOM | 2500 | CB | PHE | A | 347 | 26.876 | 44.193 | 63.795 | 1.00 | 37.17 | A | C |
| ATOM | 2501 | CG | PHE | A | 347 | 27.660 | 45.291 | 63.225 | 1.00 | 40.78 | A | C |
| ATOM | 2502 | CD1 | PHE | A | 347 | 27.694 | 45.489 | 61.855 | 1.00 | 50.69 | A | C |
| ATOM | 2503 | CD2 | PHE | A | 347 | 28.426 | 46.103 | 64.061 | 1.00 | 44.97 | A | C |
| ATOM | 2504 | CE1 | PHE | A | 347 | 28.488 | 46.491 | 61.306 | 1.00 | 55.37 | A | C |
| ATOM | 2505 | CE2 | PHE | A | 347 | 29.221 | 47.093 | 63.543 | 1.00 | 48.75 | A | C |
| ATOM | 2506 | CZ | PHE | A | 347 | 29.259 | 47.294 | 62.148 | 1.00 | 53.07 | A | C |
| ATOM | 2507 | C | PHE | A | 347 | 27.396 | 42.085 | 62.526 | 1.00 | 23.21 | A | C |
| ATOM | 2508 | O | PHE | A | 347 | 27.795 | 42.506 | 61.427 | 1.00 | 22.33 | A | O |
| ATOM | 2509 | N | SER | A | 348 | 26.821 | 40.903 | 62.659 | 1.00 | 22.86 | A | N |
| ATOM | 2510 | CA | SER | A | 348 | 26.571 | 40.137 | 61.465 | 1.00 | 28.79 | A | C |
| ATOM | 2511 | CB | SER | A | 348 | 27.278 | 38.764 | 61.481 | 1.00 | 34.45 | A | C |
| ATOM | 2512 | OG | SER | A | 348 | 27.038 | 38.033 | 62.649 | 1.00 | 32.29 | A | O |
| ATOM | 2513 | C | SER | A | 348 | 25.097 | 39.968 | 61.236 | 1.00 | 31.25 | A | C |
| ATOM | 2514 | O | SER | A | 348 | 24.242 | 40.441 | 62.012 | 1.00 | 26.68 | A | O |
| ATOM | 2515 | N | MET | A | 349 | 24.828 | 39.313 | 60.121 | 1.00 | 31.86 | A | N |
| ATOM | 2516 | CA | MET | A | 349 | 23.494 | 39.005 | 59.643 | 1.00 | 30.46 | A | C |
| ATOM | 2517 | CB | MET | A | 349 | 23.278 | 39.694 | 58.304 | 1.00 | 30.84 | A | C |
| ATOM | 2518 | CG | MET | A | 349 | 23.498 | 41.187 | 58.329 | 1.00 | 31.40 | A | C |
| ATOM | 2519 | SD | MET | A | 349 | 22.060 | 41.923 | 59.072 | 1.00 | 42.85 | A | S |
| ATOM | 2520 | CE | MET | A | 349 | 20.993 | 41.941 | 57.608 | 1.00 | 37.57 | A | C |
| ATOM | 2521 | C | MET | A | 349 | 23.583 | 37.500 | 59.434 | 1.00 | 31.62 | A | C |
| ATOM | 2522 | O | MET | A | 349 | 24.173 | 37.039 | 58.455 | 1.00 | 26.48 | A | O |
| ATOM | 2523 | N | GLU | A | 350 | 23.017 | 36.737 | 60.356 | 1.00 | 28.77 | A | N |
| ATOM | 2524 | CA | GLU | A | 350 | 23.085 | 35.301 | 60.234 | 1.00 | 29.36 | A | C |
| ATOM | 2525 | CB | GLU | A | 350 | 22.377 | 34.619 | 61.399 | 1.00 | 23.24 | A | C |
| ATOM | 2526 | CG | GLU | A | 350 | 22.731 | 33.124 | 61.489 | 1.00 | 29.89 | A | C |
| ATOM | 2527 | CD | GLU | A | 350 | 21.764 | 32.337 | 62.353 | 1.00 | 32.05 | A | C |
| ATOM | 2528 | OE1 | GLU | A | 350 | 22.151 | 31.242 | 62.813 | 1.00 | 39.90 | A | O |
| ATOM | 2529 | OE2 | GLU | A | 350 | 20.614 | 32.798 | 62.557 | 1.00 | 33.09 | A | O |
| ATOM | 2530 | C | GLU | A | 350 | 22.483 | 34.845 | 58.909 | 1.00 | 28.52 | A | C |
| ATOM | 2531 | O | GLU | A | 350 | 21.365 | 35.223 | 58.550 | 1.00 | 27.52 | A | O |
| ATOM | 2532 | N | GLY | A | 351 | 23.238 | 34.022 | 58.192 | 1.00 | 31.50 | A | N |
| ATOM | 2533 | CA | GLY | A | 351 | 22.789 | 33.535 | 56.900 | 1.00 | 31.29 | A | C |
| ATOM | 2534 | C | GLY | A | 351 | 23.352 | 34.367 | 55.758 | 1.00 | 32.73 | A | C |
| ATOM | 2535 | O | GLY | A | 351 | 23.113 | 34.067 | 54.590 | 1.00 | 34.51 | A | O |
| ATOM | 2536 | N | PHE | A | 352 | 24.106 | 35.416 | 56.072 | 1.00 | 31.29 | A | N |
| ATOM | 2537 | CA | PHE | A | 352 | 24.674 | 36.253 | 55.012 | 1.00 | 29.78 | A | C |
| ATOM | 2538 | CB | PHE | A | 352 | 23.872 | 37.545 | 54.837 | 1.00 | 30.44 | A | C |
| ATOM | 2539 | CG | PHE | A | 352 | 24.471 | 38.504 | 53.816 | 1.00 | 33.46 | A | C |
| ATOM | 2540 | CD1 | PHE | A | 352 | 24.418 | 38.220 | 52.458 | 1.00 | 37.37 | A | C |
| ATOM | 2541 | CD2 | PHE | A | 352 | 25.104 | 39.670 | 54.222 | 1.00 | 37.83 | A | C |
| ATOM | 2542 | CE1 | PHE | A | 352 | 24.987 | 39.072 | 51.516 | 1.00 | 36.94 | A | C |
| ATOM | 2543 | CE2 | PHE | A | 352 | 25.681 | 40.542 | 53.286 | 1.00 | 41.60 | A | C |
| ATOM | 2544 | CZ | PHE | A | 352 | 25.621 | 40.238 | 51.928 | 1.00 | 42.83 | A | C |
| ATOM | 2545 | C | PHE | A | 352 | 26.141 | 36.622 | 55.210 | 1.00 | 29.20 | A | C |
| ATOM | 2546 | O | PHE | A | 352 | 26.995 | 36.219 | 54.419 | 1.00 | 30.80 | A | O |
| ATOM | 2547 | N | GLY | A | 353 | 26.439 | 37.405 | 56.241 | 1.00 | 26.99 | A | N |
| ATOM | 2548 | CA | GLY | A | 353 | 27.823 | 37.813 | 56.461 | 1.00 | 30.50 | A | C |
| ATOM | 2549 | C | GLY | A | 353 | 28.046 | 38.646 | 57.714 | 1.00 | 30.44 | A | C |
| ATOM | 2550 | O | GLY | A | 353 | 27.143 | 38.786 | 58.541 | 1.00 | 28.88 | A | O |
| ATOM | 2551 | N | THR | A | 354 | 29.238 | 39.222 | 57.845 | 1.00 | 29.03 | A | N |
| ATOM | 2552 | CA | THR | A | 354 | 29.591 | 39.989 | 59.039 | 1.00 | 26.98 | A | C |
| ATOM | 2553 | CB | THR | A | 354 | 30.433 | 39.098 | 60.002 | 1.00 | 27.76 | A | C |
| ATOM | 2554 | OG1 | THR | A | 354 | 29.666 | 37.949 | 60.370 | 1.00 | 26.69 | A | O |
| ATOM | 2555 | CG2 | THR | A | 354 | 30.845 | 39.860 | 61.291 | 1.00 | 22.84 | A | C |
| ATOM | 2556 | C | THR | A | 354 | 30.380 | 41.259 | 58.731 | 1.00 | 28.07 | A | C |
| ATOM | 2557 | O | THR | A | 354 | 31.205 | 41.280 | 57.810 | 1.00 | 27.22 | A | O |
| ATOM | 2558 | N | ASP | A | 355 | 30.079 | 42.327 | 59.465 | 1.00 | 25.34 | A | N |
| ATOM | 2559 | CA | ASP | A | 355 | 30.811 | 43.590 | 59.340 | 1.00 | 28.15 | A | C |
| ATOM | 2560 | CB | ASP | A | 355 | 29.870 | 44.792 | 59.181 | 1.00 | 29.30 | A | C |
| ATOM | 2561 | CG | ASP | A | 355 | 29.279 | 44.911 | 57.781 | 1.00 | 33.13 | A | C |
| ATOM | 2562 | OD1 | ASP | A | 355 | 29.435 | 43.975 | 56.985 | 1.00 | 38.40 | A | O |
| ATOM | 2563 | OD2 | ASP | A | 355 | 28.655 | 45.953 | 57.472 | 1.00 | 37.41 | A | O |
| ATOM | 2564 | C | ASP | A | 355 | 31.614 | 43.765 | 60.645 | 1.00 | 28.99 | A | C |
| ATOM | 2565 | O | ASP | A | 355 | 31.091 | 43.605 | 61.745 | 1.00 | 32.01 | A | O |
| ATOM | 2566 | N | ALA | A | 356 | 32.899 | 44.047 | 60.516 | 1.00 | 28.36 | A | N |
| ATOM | 2567 | CA | ALA | A | 356 | 33.738 | 44.273 | 61.676 | 1.00 | 29.01 | A | C |
| ATOM | 2568 | CB | ALA | A | 356 | 34.925 | 43.307 | 61.673 | 1.00 | 27.32 | A | C |
| ATOM | 2569 | C | ALA | A | 356 | 34.202 | 45.708 | 61.472 | 1.00 | 27.85 | A | C |
| ATOM | 2570 | O | ALA | A | 356 | 34.597 | 46.078 | 60.376 | 1.00 | 29.76 | A | O |
| ATOM | 2571 | N | VAL | A | 357 | 34.130 | 46.522 | 62.513 | 1.00 | 22.20 | A | N |
| ATOM | 2572 | CA | VAL | A | 357 | 34.537 | 47.905 | 62.389 | 1.00 | 24.32 | A | C |
| ATOM | 2573 | CB | VAL | A | 357 | 33.309 | 48.860 | 62.495 | 1.00 | 24.47 | A | C |
| ATOM | 2574 | CG1 | VAL | A | 357 | 33.748 | 50.273 | 62.294 | 1.00 | 26.98 | A | C |
| ATOM | 2575 | CG2 | VAL | A | 357 | 32.249 | 48.486 | 61.456 | 1.00 | 30.11 | A | C |
| ATOM | 2576 | C | VAL | A | 357 | 35.538 | 48.319 | 63.470 | 1.00 | 23.30 | A | C |
| ATOM | 2577 | O | VAL | A | 357 | 35.335 | 48.050 | 64.646 | 1.00 | 24.67 | A | O |
| ATOM | 2578 | N | ILE | A | 358 | 36.605 | 48.994 | 63.062 | 1.00 | 23.18 | A | N |
| ATOM | 2579 | CA | ILE | A | 358 | 37.601 | 49.499 | 63.997 | 1.00 | 21.35 | A | C |
| ATOM | 2580 | CB | ILE | A | 358 | 39.041 | 49.223 | 63.519 | 1.00 | 24.19 | A | C |
| ATOM | 2581 | CG2 | ILE | A | 358 | 40.065 | 49.918 | 64.465 | 1.00 | 20.65 | A | C |
| ATOM | 2582 | CG1 | ILE | A | 358 | 39.305 | 47.716 | 63.467 | 1.00 | 22.94 | A | C |
| ATOM | 2583 | CD1 | ILE | A | 358 | 40.655 | 47.387 | 62.897 | 1.00 | 24.15 | A | C |
| ATOM | 2584 | C | ILE | A | 358 | 37.422 | 51.026 | 64.022 | 1.00 | 26.29 | A | C |
| ATOM | 2585 | O | ILE | A | 358 | 37.521 | 51.673 | 62.978 | 1.00 | 27.54 | A | O |
| ATOM | 2586 | N | TYR | A | 359 | 37.144 | 51.583 | 65.196 | 1.00 | 22.73 | A | N |
| ATOM | 2587 | CA | TYR | A | 359 | 36.993 | 53.032 | 65.359 | 1.00 | 22.10 | A | C |
| ATOM | 2588 | CB | TYR | A | 359 | 35.773 | 53.357 | 66.239 | 1.00 | 19.40 | A | C |
| ATOM | 2589 | CG | TYR | A | 359 | 34.424 | 53.011 | 65.625 | 1.00 | 20.03 | A | C |
| ATOM | 2590 | CD1 | TYR | A | 359 | 33.713 | 51.881 | 66.037 | 1.00 | 20.13 | A | C |
| ATOM | 2591 | CE1 | TYR | A | 359 | 32.493 | 51.534 | 65.443 | 1.00 | 23.75 | A | C |
| ATOM | 2592 | CD2 | TYR | A | 359 | 33.877 | 53.802 | 64.603 | 1.00 | 23.92 | A | C |
| ATOM | 2593 | CE2 | TYR | A | 359 | 32.659 | 53.475 | 64.008 | 1.00 | 23.92 | A | C |
| ATOM | 2594 | CZ | TYR | A | 359 | 31.978 | 52.338 | 64.426 | 1.00 | 28.57 | A | C |
| ATOM | 2595 | OH | TYR | A | 359 | 30.812 | 51.977 | 63.799 | 1.00 | 32.08 | A | O |
| ATOM | 2596 | C | TYR | A | 359 | 38.256 | 53.582 | 66.054 | 1.00 | 21.13 | A | C |
| ATOM | 2597 | O | TYR | A | 359 | 38.575 | 53.137 | 67.143 | 1.00 | 20.16 | A | O |
| ATOM | 2598 | N | LEU | A | 360 | 38.957 | 54.525 | 65.409 | 1.00 | 18.81 | A | N |
| ATOM | 2599 | CA | LEU | A | 360 | 40.160 | 55.176 | 65.934 | 1.00 | 21.57 | A | C |
| ATOM | 2600 | CB | LEU | A | 360 | 41.354 | 54.928 | 65.004 | 1.00 | 25.09 | A | C |
| ATOM | 2601 | CG | LEU | A | 360 | 41.716 | 53.474 | 64.675 | 1.00 | 25.94 | A | C |
| ATOM | 2602 | CD1 | LEU | A | 360 | 42.823 | 53.444 | 63.634 | 1.00 | 27.37 | A | C |
| ATOM | 2603 | CD2 | LEU | A | 360 | 42.149 | 52.749 | 65.940 | 1.00 | 23.97 | A | C |
| ATOM | 2604 | C | LEU | A | 360 | 39.912 | 56.693 | 65.978 | 1.00 | 27.02 | A | C |
| ATOM | 2605 | O | LEU | A | 360 | 39.044 | 57.209 | 65.251 | 1.00 | 27.07 | A | O |
| ATOM | 2606 | N | LYS | A | 361 | 40.663 | 57.415 | 66.811 | 1.00 | 27.24 | A | N |
| ATOM | 2607 | CA | LYS | A | 361 | 40.496 | 58.865 | 66.866 | 1.00 | 30.44 | A | C |
| ATOM | 2608 | CB | LYS | A | 361 | 41.040 | 59.447 | 68.166 | 1.00 | 29.78 | A | C |
| ATOM | 2609 | CG | LYS | A | 361 | 40.264 | 59.094 | 69.423 | 1.00 | 33.37 | A | C |
| ATOM | 2610 | CD | LYS | A | 361 | 38.819 | 59.599 | 69.394 | 1.00 | 34.06 | A | C |
| ATOM | 2611 | CE | LYS | A | 361 | 38.209 | 59.477 | 70.788 | 1.00 | 31.65 | A | C |
| ATOM | 2612 | NZ | LYS | A | 361 | 36.797 | 59.908 | 70.839 | 1.00 | 35.69 | A | N |
| ATOM | 2613 | C | LYS | A | 361 | 41.285 | 59.433 | 65.696 | 1.00 | 30.83 | A | C |
| ATOM | 2614 | O | LYS | A | 361 | 42.452 | 59.086 | 65.499 | 1.00 | 30.34 | A | O |
| ATOM | 2615 | N | ALA | A | 362 | 40.649 | 60.294 | 64.915 | 1.00 | 29.84 | A | N |
| ATOM | 2616 | CA | ALA | A | 362 | 41.302 | 60.903 | 63.759 | 1.00 | 32.17 | A | C |
| ATOM | 2617 | CB | ALA | A | 362 | 40.260 | 61.647 | 62.914 | 1.00 | 35.62 | A | C |
| ATOM | 2618 | C | ALA | A | 362 | 42.438 | 61.866 | 64.132 | 1.00 | 36.55 | A | C |
| ATOM | 2619 | O | ALA | A | 362 | 43.458 | 61.938 | 63.442 | 1.00 | 39.16 | A | O |
| ATOM | 2620 | N | LEU | A | 363 | 42.257 | 62.597 | 65.227 | 1.00 | 38.29 | A | N |
| ATOM | 2621 | CA | LEU | A | 363 | 43.232 | 63.593 | 65.664 | 1.00 | 40.28 | A | C |
| ATOM | 2622 | CB | LEU | A | 363 | 42.485 | 64.859 | 66.106 | 1.00 | 41.11 | A | C |
| ATOM | 2623 | CG | LEU | A | 363 | 41.366 | 65.304 | 65.150 | 1.00 | 48.81 | A | C |
| ATOM | 2624 | CD1 | LEU | A | 363 | 40.690 | 66.557 | 65.697 | 1.00 | 48.38 | A | C |
| ATOM | 2625 | CD2 | LEU | A | 363 | 41.937 | 65.558 | 63.757 | 1.00 | 46.93 | A | C |
| ATOM | 2626 | C | LEU | A | 363 | 44.186 | 63.155 | 66.777 | 1.00 | 41.02 | A | C |
| ATOM | 2627 | O | LEU | A | 363 | 43.775 | 62.564 | 67.776 | 1.00 | 38.36 | A | O |
| ATOM | 2628 | N | SER | A | 364 | 45.466 | 63.468 | 66.597 | 1.00 | 40.56 | A | N |
| ATOM | 2629 | CA | SER | A | 364 | 46.497 | 63.136 | 67.582 | 1.00 | 45.74 | A | C |
| ATOM | 2630 | CB | SER | A | 364 | 47.857 | 63.673 | 67.120 | 1.00 | 43.32 | A | C |
| ATOM | 2631 | OG | SER | A | 364 | 47.762 | 65.050 | 66.800 | 1.00 | 48.12 | A | O |
| ATOM | 2632 | C | SER | A | 364 | 46.137 | 63.734 | 68.948 | 1.00 | 46.39 | A | C |
| ATOM | 2633 | O | SER | A | 364 | 46.578 | 63.253 | 69.994 | 1.00 | 49.04 | A | O |
| ATOM | 2634 | N | THR | A | 365 | 45.319 | 64.776 | 68.920 | 1.00 | 47.62 | A | N |
| ATOM | 2635 | CA | THR | A | 365 | 44.864 | 65.459 | 70.122 | 1.00 | 49.41 | A | C |
| ATOM | 2636 | CB | THR | A | 365 | 44.051 | 66.695 | 69.741 | 1.00 | 52.09 | A | C |
| ATOM | 2637 | OG1 | THR | A | 365 | 44.840 | 67.538 | 68.890 | 1.00 | 55.48 | A | O |
| ATOM | 2638 | CG2 | THR | A | 365 | 43.619 | 67.447 | 70.982 | 1.00 | 56.19 | A | C |
| ATOM | 2639 | C | THR | A | 365 | 43.975 | 64.588 | 71.019 | 1.00 | 49.16 | A | C |
| ATOM | 2640 | O | THR | A | 365 | 44.127 | 64.582 | 72.243 | 1.00 | 47.41 | A | O |
| ATOM | 2641 | N | ASP | A | 366 | 43.037 | 63.868 | 70.407 | 1.00 | 45.26 | A | N |
| ATOM | 2642 | CA | ASP | A | 366 | 42.110 | 63.028 | 71.164 | 1.00 | 45.11 | A | C |
| ATOM | 2643 | CB | ASP | A | 366 | 40.783 | 62.898 | 70.410 | 1.00 | 48.38 | A | C |
| ATOM | 2644 | CG | ASP | A | 366 | 40.207 | 64.245 | 70.001 | 1.00 | 52.42 | A | C |
| ATOM | 2645 | OD1 | ASP | A | 366 | 40.096 | 65.139 | 70.866 | 1.00 | 56.11 | A | O |
| ATOM | 2646 | OD2 | ASP | A | 366 | 39.861 | 64.408 | 68.814 | 1.00 | 57.08 | A | O |
| ATOM | 2647 | C | ASP | A | 366 | 42.652 | 61.629 | 71.450 | 1.00 | 42.48 | A | C |
| ATOM | 2648 | O | ASP | A | 366 | 42.000 | 60.826 | 72.120 | 1.00 | 36.90 | A | O |
| ATOM | 2649 | N | SER | A | 367 | 43.837 | 61.342 | 70.932 | 1.00 | 38.30 | A | N |
| ATOM | 2650 | CA | SER | A | 367 | 44.455 | 60.042 | 71.123 | 1.00 | 44.45 | A | C |
| ATOM | 2651 | CB | SER | A | 367 | 45.368 | 59.721 | 69.936 | 1.00 | 44.24 | A | C |
| ATOM | 2652 | OG | SER | A | 367 | 44.633 | 59.734 | 68.718 | 1.00 | 49.01 | A | O |
| ATOM | 2653 | C | SER | A | 367 | 45.255 | 60.029 | 72.424 | 1.00 | 46.76 | A | C |
| ATOM | 2654 | O | SER | A | 367 | 46.434 | 60.402 | 72.446 | 1.00 | 47.21 | A | O |
| ATOM | 2655 | N | VAL | A | 368 | 44.605 | 59.587 | 73.499 | 1.00 | 45.19 | A | N |
| ATOM | 2656 | CA | VAL | A | 368 | 45.238 | 59.539 | 74.805 | 1.00 | 44.95 | A | C |
| ATOM | 2657 | CB | VAL | A | 368 | 44.636 | 60.608 | 75.753 | 1.00 | 45.86 | A | C |
| ATOM | 2658 | CG1 | VAL | A | 368 | 45.425 | 60.656 | 77.037 | 1.00 | 45.94 | A | C |
| ATOM | 2659 | CG2 | VAL | A | 368 | 44.650 | 61.977 | 75.082 | 1.00 | 46.38 | A | C |
| ATOM | 2660 | C | VAL | A | 368 | 45.079 | 58.170 | 75.454 | 1.00 | 44.38 | A | C |
| ATOM | 2661 | O | VAL | A | 368 | 44.013 | 57.552 | 75.387 | 1.00 | 44.85 | A | O |
| ATOM | 2662 | N | GLU | A | 369 | 46.147 | 57.703 | 76.086 | 1.00 | 42.62 | A | N |
| ATOM | 2663 | CA | GLU | A | 369 | 46.137 | 56.411 | 76.761 | 1.00 | 41.22 | A | C |
| ATOM | 2664 | CB | GLU | A | 369 | 47.376 | 56.260 | 77.646 | 1.00 | 38.30 | A | C |
| ATOM | 2665 | CG | GLU | A | 369 | 48.684 | 56.095 | 76.908 | 1.00 | 36.74 | A | C |
| ATOM | 2666 | CD | GLU | A | 369 | 49.795 | 55.691 | 77.857 | 1.00 | 39.15 | A | C |
| ATOM | 2667 | OE1 | GLU | A | 369 | 50.207 | 56.540 | 78.680 | 1.00 | 40.85 | A | O |
| ATOM | 2668 | OE2 | GLU | A | 369 | 50.236 | 54.520 | 77.797 | 1.00 | 32.84 | A | O |
| ATOM | 2669 | C | GLU | A | 369 | 44.900 | 56.194 | 77.626 | 1.00 | 40.44 | A | C |
| ATOM | 2670 | O | GLU | A | 369 | 44.453 | 57.088 | 78.337 | 1.00 | 43.03 | A | O |
| ATOM | 2671 | N | ARG | A | 370 | 44.365 | 54.986 | 77.556 | 1.00 | 39.21 | A | N |
| ATOM | 2672 | CA | ARG | A | 370 | 43.200 | 54.574 | 78.329 | 1.00 | 44.84 | A | C |
| ATOM | 2673 | CB | ARG | A | 370 | 42.226 | 53.865 | 77.386 | 1.00 | 48.30 | A | C |
| ATOM | 2674 | CG | ARG | A | 370 | 41.159 | 53.017 | 78.026 | 1.00 | 52.36 | A | C |
| ATOM | 2675 | CD | ARG | A | 370 | 40.042 | 53.837 | 78.606 | 1.00 | 59.26 | A | C |
| ATOM | 2676 | NE | ARG | A | 370 | 38.938 | 52.968 | 79.001 | 1.00 | 68.33 | A | N |
| ATOM | 2677 | CZ | ARG | A | 370 | 37.863 | 53.375 | 79.669 | 1.00 | 72.24 | A | C |
| ATOM | 2678 | NH1 | ARG | A | 370 | 37.743 | 54.651 | 80.023 | 1.00 | 71.95 | A | N |
| ATOM | 2679 | NH2 | ARG | A | 370 | 36.909 | 52.504 | 79.982 | 1.00 | 73.00 | A | N |
| ATOM | 2680 | C | ARG | A | 370 | 43.810 | 53.605 | 79.347 | 1.00 | 45.61 | A | C |
| ATOM | 2681 | O | ARG | A | 370 | 44.171 | 52.482 | 78.985 | 1.00 | 46.48 | A | O |
| ATOM | 2682 | N | LEU | A | 371 | 43.933 | 54.042 | 80.607 | 1.00 | 44.11 | A | N |
| ATOM | 2683 | CA | LEU | A | 371 | 44.581 | 53.243 | 81.653 | 1.00 | 40.67 | A | C |
| ATOM | 2684 | CB | LEU | A | 371 | 45.838 | 53.953 | 82.134 | 1.00 | 38.48 | A | C |
| ATOM | 2685 | CG | LEU | A | 371 | 46.872 | 54.393 | 81.113 | 1.00 | 38.25 | A | C |
| ATOM | 2686 | CD1 | LEU | A | 371 | 47.889 | 55.269 | 81.816 | 1.00 | 41.63 | A | C |
| ATOM | 2687 | CD2 | LEU | A | 371 | 47.552 | 53.184 | 80.483 | 1.00 | 40.68 | A | C |
| ATOM | 2688 | C | LEU | A | 371 | 43.764 | 52.931 | 82.879 | 1.00 | 40.68 | A | C |
| ATOM | 2689 | O | LEU | A | 371 | 43.141 | 53.805 | 83.454 | 1.00 | 41.61 | A | O |
| ATOM | 2690 | N | PRO | A | 372 | 43.790 | 51.670 | 83.328 | 1.00 | 43.33 | A | N |
| ATOM | 2691 | CD | PRO | A | 372 | 44.544 | 50.518 | 82.799 | 1.00 | 44.81 | A | C |
| ATOM | 2692 | CA | PRO | A | 372 | 43.026 | 51.297 | 84.519 | 1.00 | 45.00 | A | C |
| ATOM | 2693 | CB | PRO | A | 372 | 43.012 | 49.776 | 84.444 | 1.00 | 45.51 | A | C |
| ATOM | 2694 | CG | PRO | A | 372 | 44.383 | 49.479 | 83.903 | 1.00 | 44.19 | A | C |
| ATOM | 2695 | C | PRO | A | 372 | 43.741 | 51.812 | 85.776 | 1.00 | 46.80 | A | C |
| ATOM | 2696 | O | PRO | A | 372 | 44.954 | 52.006 | 85.766 | 1.00 | 41.86 | A | O |
| ATOM | 2697 | N | VAL | A | 373 | 42.984 | 52.041 | 86.845 | 1.00 | 54.20 | A | N |
| ATOM | 2698 | CA | VAL | A | 373 | 43.544 | 52.518 | 88.109 | 1.00 | 62.33 | A | C |
| ATOM | 2699 | CB | VAL | A | 373 | 43.318 | 54.026 | 88.316 | 1.00 | 64.06 | A | C |
| ATOM | 2700 | CG1 | VAL | A | 373 | 43.833 | 54.456 | 89.693 | 1.00 | 61.43 | A | C |
| ATOM | 2701 | CG2 | VAL | A | 373 | 44.030 | 54.792 | 87.242 | 1.00 | 65.34 | A | C |
| ATOM | 2702 | C | VAL | A | 373 | 42.870 | 51.806 | 89.258 | 1.00 | 66.84 | A | C |
| ATOM | 2703 | O | VAL | A | 373 | 41.654 | 51.610 | 89.246 | 1.00 | 64.96 | A | O |
| ATOM | 2704 | N | TYR | A | 374 | 43.660 | 51.424 | 90.255 | 1.00 | 73.80 | A | N |
| ATOM | 2705 | CA | TYR | A | 374 | 43.112 | 50.735 | 91.412 | 1.00 | 81.20 | A | C |
| ATOM | 2706 | CB | TYR | A | 374 | 44.202 | 49.932 | 92.131 | 1.00 | 87.30 | A | C |
| ATOM | 2707 | CG | TYR | A | 374 | 43.703 | 49.355 | 93.429 | 1.00 | 94.36 | A | C |
| ATOM | 2708 | CD1 | TYR | A | 374 | 42.624 | 48.465 | 93.446 | 1.00 | 97.84 | A | C |
| ATOM | 2709 | CE1 | TYR | A | 374 | 42.072 | 48.019 | 94.648 | 1.00 | 100.79 | A | C |
| ATOM | 2710 | CD2 | TYR | A | 374 | 44.230 | 49.778 | 94.647 | 1.00 | 96.95 | A | C |
| ATOM | 2711 | CE2 | TYR | A | 374 | 43.688 | 49.340 | 95.856 | 1.00 | 101.22 | A | C |
| ATOM | 2712 | CZ | TYR | A | 374 | 42.605 | 48.464 | 95.849 | 1.00 | 102.25 | A | C |
| ATOM | 2713 | OH | TYR | A | 374 | 42.033 | 48.065 | 97.039 | 1.00 | 103.94 | A | O |
| ATOM | 2714 | C | TYR | A | 374 | 42.445 | 51.701 | 92.397 | 1.00 | 81.47 | A | C |
| ATOM | 2715 | O | TYR | A | 374 | 43.114 | 52.510 | 93.037 | 1.00 | 78.35 | A | O |
| ATOM | 2716 | N | ASN | A | 375 | 41.121 | 51.602 | 92.505 | 1.00 | 84.50 | A | N |
| ATOM | 2717 | CA | ASN | A | 375 | 40.334 | 52.447 | 93.405 | 1.00 | 87.85 | A | C |
| ATOM | 2718 | CB | ASN | A | 375 | 39.519 | 53.487 | 92.626 | 1.00 | 88.01 | A | C |
| ATOM | 2719 | CG | ASN | A | 375 | 40.375 | 54.497 | 91.919 | 1.00 | 88.40 | A | C |
| ATOM | 2720 | OD1 | ASN | A | 375 | 41.352 | 54.995 | 92.470 | 1.00 | 90.49 | A | O |
| ATOM | 2721 | ND2 | ASN | A | 375 | 40.004 | 54.825 | 90.694 | 1.00 | 88.34 | A | N |
| ATOM | 2722 | C | ASN | A | 375 | 39.337 | 51.625 | 94.204 | 1.00 | 89.94 | A | C |
| ATOM | 2723 | O | ASN | A | 375 | 39.296 | 50.395 | 94.112 | 1.00 | 89.88 | A | O |
| ATOM | 2724 | N | LYS | A | 376 | 38.529 | 52.334 | 94.989 | 1.00 | 92.48 | A | N |
| ATOM | 2725 | CA | LYS | A | 376 | 37.467 | 51.723 | 95.774 | 1.00 | 94.92 | A | C |
| ATOM | 2726 | CB | LYS | A | 376 | 36.946 | 52.719 | 96.817 | 1.00 | 94.48 | A | C |
| ATOM | 2727 | CG | LYS | A | 376 | 35.469 | 52.567 | 97.175 | 1.00 | 95.16 | A | C |
| ATOM | 2728 | CD | LYS | A | 376 | 35.152 | 51.211 | 97.788 | 1.00 | 96.88 | A | C |
| ATOM | 2729 | CE | LYS | A | 376 | 33.647 | 51.021 | 97.948 | 1.00 | 98.17 | A | C |
| ATOM | 2730 | NZ | LYS | A | 376 | 33.030 | 52.078 | 98.802 | 1.00 | 99.91 | A | N |
| ATOM | 2731 | C | LYS | A | 376 | 36.395 | 51.430 | 94.722 | 1.00 | 96.82 | A | C |
| ATOM | 2732 | O | LYS | A | 376 | 35.533 | 50.560 | 94.896 | 1.00 | 97.01 | A | O |
| ATOM | 2733 | N | SER | A | 377 | 36.483 | 52.176 | 93.622 | 1.00 | 98.02 | A | N |
| ATOM | 2734 | CA | SER | A | 377 | 35.574 | 52.049 | 92.489 | 1.00 | 99.12 | A | C |
| ATOM | 2735 | CB | SER | A | 377 | 35.859 | 53.162 | 91.474 | 1.00 | 96.70 | A | C |
| ATOM | 2736 | OG | SER | A | 377 | 35.108 | 52.985 | 90.287 | 1.00 | 92.69 | A | O |
| ATOM | 2737 | C | SER | A | 377 | 35.772 | 50.688 | 91.829 | 1.00 | 101.51 | A | C |
| ATOM | 2738 | O | SER | A | 377 | 34.811 | 49.970 | 91.543 | 1.00 | 100.65 | A | O |
| ATOM | 2739 | N | ALA | A | 378 | 37.034 | 50.344 | 91.593 | 1.00 | 104.41 | A | N |
| ATOM | 2740 | CA | ALA | A | 378 | 37.383 | 49.077 | 90.973 | 1.00 | 108.08 | A | C |
| ATOM | 2741 | CB | ALA | A | 378 | 38.863 | 49.073 | 90.608 | 1.00 | 106.32 | A | C |
| ATOM | 2742 | C | ALA | A | 378 | 37.069 | 47.903 | 91.898 | 1.00 | 111.08 | A | C |
| ATOM | 2743 | O | ALA | A | 378 | 36.621 | 46.845 | 91.448 | 1.00 | 111.10 | A | O |
| ATOM | 2744 | N | TRP | A | 379 | 37.300 | 48.100 | 93.194 | 1.00 | 114.71 | A | N |
| ATOM | 2745 | CA | TRP | A | 379 | 37.064 | 47.059 | 94.187 | 1.00 | 117.87 | A | C |
| ATOM | 2746 | CB | TRP | A | 379 | 37.302 | 47.601 | 95.599 | 1.00 | 124.80 | A | C |
| ATOM | 2747 | CG | TRP | A | 379 | 37.197 | 46.529 | 96.644 | 1.00 | 134.10 | A | C |
| ATOM | 2748 | CD2 | TRP | A | 379 | 36.052 | 46.228 | 97.456 | 1.00 | 137.89 | A | C |
| ATOM | 2749 | CE2 | TRP | A | 379 | 36.377 | 45.090 | 98.233 | 1.00 | 139.24 | A | C |
| ATOM | 2750 | CE3 | TRP | A | 379 | 34.782 | 46.805 | 97.601 | 1.00 | 139.57 | A | C |
| ATOM | 2751 | CD1 | TRP | A | 379 | 38.143 | 45.593 | 96.956 | 1.00 | 137.00 | A | C |
| ATOM | 2752 | NE1 | TRP | A | 379 | 37.658 | 44.725 | 97.908 | 1.00 | 138.93 | A | N |
| ATOM | 2753 | CZ2 | TRP | A | 379 | 35.479 | 44.518 | 99.141 | 1.00 | 140.42 | A | C |
| ATOM | 2754 | CZ3 | TRP | A | 379 | 33.887 | 46.235 | 98.505 | 1.00 | 140.63 | A | C |
| ATOM | 2755 | CH2 | TRP | A | 379 | 34.243 | 45.102 | 99.262 | 1.00 | 141.17 | A | C |
| ATOM | 2756 | C | TRP | A | 379 | 35.672 | 46.421 | 94.136 | 1.00 | 116.17 | A | C |
| ATOM | 2757 | O | TRP | A | 379 | 35.552 | 45.194 | 94.189 | 1.00 | 116.27 | A | O |
| ATOM | 2758 | N | ARG | A | 380 | 34.626 | 47.240 | 94.032 | 1.00 | 112.98 | A | N |
| ATOM | 2759 | CA | ARG | A | 380 | 33.262 | 46.714 | 94.014 | 1.00 | 109.21 | A | C |
| ATOM | 2760 | CB | ARG | A | 380 | 32.259 | 47.810 | 94.402 | 1.00 | 109.97 | A | C |
| ATOM | 2761 | CG | ARG | A | 380 | 32.465 | 49.166 | 93.760 | 1.00 | 109.96 | A | C |
| ATOM | 2762 | CD | ARG | A | 380 | 31.720 | 50.218 | 94.576 | 1.00 | 111.04 | A | C |
| ATOM | 2763 | NE | ARG | A | 380 | 31.880 | 51.574 | 94.057 | 1.00 | 112.39 | A | N |
| ATOM | 2764 | CZ | ARG | A | 380 | 31.466 | 52.670 | 94.689 | 1.00 | 112.89 | A | C |
| ATOM | 2765 | NH1 | ARG | A | 380 | 30.868 | 52.571 | 95.870 | 1.00 | 111.77 | A | N |
| ATOM | 2766 | NH2 | ARG | A | 380 | 31.645 | 53.865 | 94.138 | 1.00 | 112.93 | A | N |
| ATOM | 2767 | C | ARG | A | 380 | 32.792 | 45.993 | 92.753 | 1.00 | 106.08 | A | C |
| ATOM | 2768 | O | ARG | A | 380 | 31.680 | 45.461 | 92.720 | 1.00 | 104.51 | A | O |
| ATOM | 2769 | N | HIS | A | 381 | 33.625 | 45.967 | 91.718 | 1.00 | 103.49 | A | N |
| ATOM | 2770 | CA | HIS | A | 381 | 33.262 | 45.251 | 90.497 | 1.00 | 100.91 | A | C |
| ATOM | 2771 | CB | HIS | A | 381 | 34.123 | 45.706 | 89.314 | 1.00 | 102.58 | A | C |
| ATOM | 2772 | CG | HIS | A | 381 | 33.666 | 46.986 | 88.685 | 1.00 | 105.00 | A | C |
| ATOM | 2773 | CD2 | HIS | A | 381 | 33.140 | 47.239 | 87.462 | 1.00 | 105.75 | A | C |
| ATOM | 2774 | ND1 | HIS | A | 381 | 33.720 | 48.200 | 89.336 | 1.00 | 106.42 | A | N |
| ATOM | 2775 | CE1 | HIS | A | 381 | 33.249 | 49.146 | 88.542 | 1.00 | 106.69 | A | C |
| ATOM | 2776 | NE2 | HIS | A | 381 | 32.890 | 48.589 | 87.399 | 1.00 | 106.21 | A | N |
| ATOM | 2777 | C | HIS | A | 381 | 33.486 | 43.756 | 90.750 | 1.00 | 98.09 | A | C |
| ATOM | 2778 | O | HIS | A | 381 | 33.059 | 42.907 | 89.967 | 1.00 | 96.45 | A | O |
| ATOM | 2779 | N | TYR | A | 382 | 34.154 | 43.457 | 91.865 | 1.00 | 95.62 | A | N |
| ATOM | 2780 | CA | TYR | A | 382 | 34.477 | 42.089 | 92.288 | 1.00 | 93.35 | A | C |
| ATOM | 2781 | CB | TYR | A | 382 | 35.934 | 42.000 | 92.728 | 1.00 | 88.35 | A | C |
| ATOM | 2782 | CG | TYR | A | 382 | 36.951 | 42.340 | 91.671 | 1.00 | 84.24 | A | C |
| ATOM | 2783 | CD1 | TYR | A | 382 | 37.135 | 41.515 | 90.557 | 1.00 | 82.99 | A | C |
| ATOM | 2784 | CE1 | TYR | A | 382 | 38.109 | 41.810 | 89.589 | 1.00 | 82.16 | A | C |
| ATOM | 2785 | CD2 | TYR | A | 382 | 37.758 | 43.474 | 91.797 | 1.00 | 82.80 | A | C |
| ATOM | 2786 | CE2 | TYR | A | 382 | 38.730 | 43.779 | 90.840 | 1.00 | 81.61 | A | C |
| ATOM | 2787 | CZ | TYR | A | 382 | 38.900 | 42.944 | 89.742 | 1.00 | 82.20 | A | C |
| ATOM | 2788 | OH | TYR | A | 382 | 39.860 | 43.248 | 88.805 | 1.00 | 80.26 | A | O |
| ATOM | 2789 | C | TYR | A | 382 | 33.619 | 41.668 | 93.473 | 1.00 | 94.56 | A | C |
| ATOM | 2790 | O | TYR | A | 382 | 33.917 | 40.680 | 94.159 | 1.00 | 93.28 | A | O |
| ATOM | 2791 | N | GLN | A | 383 | 32.572 | 42.437 | 93.739 | 1.00 | 95.32 | A | N |
| ATOM | 2792 | CA | GLN | A | 383 | 31.696 | 42.124 | 94.857 | 1.00 | 96.55 | A | C |
| ATOM | 2793 | CB | GLN | A | 383 | 31.398 | 43.392 | 95.655 | 1.00 | 96.45 | A | C |
| ATOM | 2794 | CG | GLN | A | 383 | 32.662 | 44.047 | 96.185 | 1.00 | 97.22 | A | C |
| ATOM | 2795 | CD | GLN | A | 383 | 33.617 | 43.018 | 96.803 | 1.00 | 96.75 | A | C |
| ATOM | 2796 | OE1 | GLN | A | 383 | 34.712 | 42.756 | 96.277 | 1.00 | 95.42 | A | O |
| ATOM | 2797 | NE2 | GLN | A | 383 | 33.191 | 42.415 | 97.915 | 1.00 | 95.23 | A | N |
| ATOM | 2798 | C | GLN | A | 383 | 30.408 | 41.473 | 94.418 | 1.00 | 97.19 | A | C |
| ATOM | 2799 | O | GLN | A | 383 | 29.482 | 41.307 | 95.212 | 1.00 | 97.27 | A | O |
| ATOM | 2800 | N | THR | A | 384 | 30.357 | 41.117 | 93.141 | 1.00 | 97.97 | A | N |
| ATOM | 2801 | CA | THR | A | 384 | 29.187 | 40.472 | 92.587 | 1.00 | 98.00 | A | C |
| ATOM | 2802 | CB | THR | A | 384 | 29.411 | 40.056 | 91.112 | 1.00 | 98.59 | A | C |
| ATOM | 2803 | OG1 | THR | A | 384 | 30.232 | 41.033 | 90.456 | 1.00 | 99.82 | A | O |
| ATOM | 2804 | CG2 | THR | A | 384 | 28.077 | 39.954 | 90.367 | 1.00 | 97.11 | A | C |
| ATOM | 2805 | C | THR | A | 384 | 28.935 | 39.216 | 93.411 | 1.00 | 98.72 | A | C |
| ATOM | 2806 | O | THR | A | 384 | 29.828 | 38.408 | 93.656 | 1.00 | 98.24 | A | O |
| ATOM | 2807 | N | ILE | A | 385 | 27.695 | 39.095 | 93.854 | 1.00 | 98.97 | A | N |
| ATOM | 2808 | CA | ILE | A | 385 | 27.188 | 37.974 | 94.638 | 1.00 | 98.73 | A | C |
| ATOM | 2809 | CB | ILE | A | 385 | 26.843 | 38.432 | 96.071 | 1.00 | 98.16 | A | C |
| ATOM | 2810 | CG2 | ILE | A | 385 | 26.480 | 37.238 | 96.954 | 1.00 | 97.80 | A | C |
| ATOM | 2811 | CG1 | ILE | A | 385 | 28.054 | 39.143 | 96.681 | 1.00 | 96.75 | A | C |
| ATOM | 2812 | CD1 | ILE | A | 385 | 27.650 | 40.213 | 97.660 | 1.00 | 94.58 | A | C |
| ATOM | 2813 | C | ILE | A | 385 | 25.906 | 37.614 | 93.902 | 1.00 | 99.57 | A | C |
| ATOM | 2814 | O | ILE | A | 385 | 24.826 | 37.583 | 94.484 | 1.00 | 99.94 | A | O |
| ATOM | 2815 | N | ALA | A | 386 | 26.032 | 37.361 | 92.605 | 1.00 | 99.08 | A | N |
| ATOM | 2816 | CA | ALA | A | 386 | 24.865 | 37.033 | 91.800 | 1.00 | 98.16 | A | C |
| ATOM | 2817 | CB | ALA | A | 386 | 25.254 | 36.848 | 90.334 | 1.00 | 98.90 | A | C |
| ATOM | 2818 | C | ALA | A | 386 | 24.111 | 35.809 | 92.284 | 1.00 | 97.12 | A | C |
| ATOM | 2819 | O | ALA | A | 386 | 24.707 | 34.848 | 92.772 | 1.00 | 96.26 | A | O |
| ATOM | 2820 | N | ALA | A | 387 | 22.788 | 35.875 | 92.135 | 1.00 | 95.21 | A | N |
| ATOM | 2821 | CA | ALA | A | 387 | 21.867 | 34.810 | 92.522 | 1.00 | 93.12 | A | C |
| ATOM | 2822 | CB | ALA | A | 387 | 22.167 | 34.352 | 93.954 | 1.00 | 93.31 | A | C |
| ATOM | 2823 | C | ALA | A | 387 | 20.424 | 35.332 | 92.419 | 1.00 | 92.44 | A | C |
| ATOM | 2824 | O | ALA | A | 387 | 19.832 | 35.696 | 93.435 | 1.00 | 92.34 | A | O |
| ATOM | 2825 | N | ALA | A | 388 | 19.898 | 35.393 | 91.187 | 1.00 | 90.92 | A | N |
| ATOM | 2826 | CA | ALA | A | 388 | 18.530 | 35.847 | 90.869 | 1.00 | 88.64 | A | C |
| ATOM | 2827 | CB | ALA | A | 388 | 17.636 | 35.740 | 92.102 | 1.00 | 86.54 | A | C |
| ATOM | 2828 | C | ALA | A | 388 | 18.455 | 37.267 | 90.286 | 1.00 | 87.54 | A | C |
| ATOM | 2829 | O | ALA | A | 388 | 18.470 | 38.245 | 91.037 | 1.00 | 87.86 | A | O |
| ATOM | 2830 | N | GLY | A | 389 | 18.329 | 37.361 | 88.958 | 1.00 | 86.36 | A | N |
| ATOM | 2831 | CA | GLY | A | 389 | 18.281 | 38.643 | 88.272 | 1.00 | 86.21 | A | C |
| ATOM | 2832 | C | GLY | A | 389 | 17.070 | 39.550 | 88.398 | 1.00 | 85.10 | A | C |
| ATOM | 2833 | O | GLY | A | 389 | 16.058 | 39.223 | 88.997 | 1.00 | 85.15 | A | O |
| ATOM | 2834 | N | ASP | A | 390 | 17.200 | 40.724 | 87.795 | 1.00 | 83.42 | A | N |
| ATOM | 2835 | CA | ASP | A | 390 | 16.165 | 41.757 | 87.795 | 1.00 | 79.95 | A | C |
| ATOM | 2836 | CB | ASP | A | 390 | 16.853 | 43.124 | 87.789 | 1.00 | 78.45 | A | C |
| ATOM | 2837 | CG | ASP | A | 390 | 18.034 | 43.191 | 86.826 | 1.00 | 79.45 | A | C |
| ATOM | 2838 | OD1 | ASP | A | 390 | 18.543 | 42.124 | 86.405 | 1.00 | 79.08 | A | O |
| ATOM | 2839 | OD2 | ASP | A | 390 | 18.468 | 44.327 | 86.488 | 1.00 | 74.89 | A | O |
| ATOM | 2840 | C | ASP | A | 390 | 15.167 | 41.616 | 86.638 | 1.00 | 78.46 | A | C |
| ATOM | 2841 | O | ASP | A | 390 | 14.210 | 42.369 | 86.513 | 1.00 | 75.98 | A | O |
| ATOM | 2842 | N | TRP | A | 391 | 15.410 | 40.627 | 85.793 | 1.00 | 78.42 | A | N |
| ATOM | 2843 | CA | TRP | A | 391 | 14.524 | 40.335 | 84.681 | 1.00 | 80.37 | A | C |
| ATOM | 2844 | CB | TRP | A | 391 | 15.258 | 40.531 | 83.347 | 1.00 | 77.76 | A | C |
| ATOM | 2845 | CG | TRP | A | 391 | 15.924 | 41.892 | 83.223 | 1.00 | 76.45 | A | C |
| ATOM | 2846 | CD2 | TRP | A | 391 | 15.364 | 43.066 | 82.623 | 1.00 | 74.85 | A | C |
| ATOM | 2847 | CE2 | TRP | A | 391 | 16.309 | 44.112 | 82.782 | 1.00 | 74.79 | A | C |
| ATOM | 2848 | CE3 | TRP | A | 391 | 14.153 | 43.338 | 81.961 | 1.00 | 73.30 | A | C |
| ATOM | 2849 | CD1 | TRP | A | 391 | 17.162 | 42.266 | 83.711 | 1.00 | 75.17 | A | C |
| ATOM | 2850 | NE1 | TRP | A | 391 | 17.395 | 43.597 | 83.446 | 1.00 | 73.76 | A | N |
| ATOM | 2851 | CZ2 | TRP | A | 391 | 16.074 | 45.411 | 82.310 | 1.00 | 73.41 | A | C |
| ATOM | 2852 | CZ3 | TRP | A | 391 | 13.922 | 44.633 | 81.491 | 1.00 | 71.95 | A | C |
| ATOM | 2853 | CH2 | TRP | A | 391 | 14.881 | 45.650 | 81.668 | 1.00 | 72.87 | A | C |
| ATOM | 2854 | C | TRP | A | 391 | 14.100 | 38.884 | 84.864 | 1.00 | 82.85 | A | C |
| ATOM | 2855 | O | TRP | A | 391 | 14.819 | 38.100 | 85.473 | 1.00 | 82.52 | A | O |
| ATOM | 2856 | N | CYS | A | 392 | 12.926 | 38.533 | 84.354 | 1.00 | 86.54 | A | N |
| ATOM | 2857 | CA | CYS | A | 392 | 12.427 | 37.159 | 84.473 | 1.00 | 91.18 | A | C |
| ATOM | 2858 | CB | CYS | A | 392 | 11.300 | 37.097 | 85.506 | 1.00 | 91.08 | A | C |
| ATOM | 2859 | SG | CYS | A | 392 | 9.862 | 38.121 | 85.047 | 1.00 | 92.03 | A | S |
| ATOM | 2860 | C | CYS | A | 392 | 11.937 | 36.587 | 83.143 | 1.00 | 93.71 | A | C |
| ATOM | 2861 | O | CYS | A | 392 | 12.744 | 36.539 | 82.186 | 1.00 | 95.60 | A | O |
| ATOM | 2862 | OXT | CYS | A | 392 | 10.754 | 36.190 | 83.079 | 1.00 | 98.21 | A | O |
| TER | 2863 | | CYS | A | 392 | | | | | | A | |
| END | | | | | | | | | | | | |

TABLE 4
Atomic coordinates for PDHK-2* with Compound 1

REMARK coordinates from restrained individual B-factor refinement
REMARK refinement resolution: 30 - 2.35 A

| | | |
|---|---|---|
| REMARK starting | r = 0.2374 | free_r = 0.2559 |
| REMARK final | r = 0.2371 | free_r = 0.2554 |

| | | |
|---|---|---|
| REMARK B rmsd for bonded mainchain atoms = | 2.562 | target = 1.5 |
| REMARK B rmsd for bonded sidechain atoms = | 3.784 | target = 2.0 |
| REMARK B rmsd for angle mainchain atoms = | 4.397 | target = 2.0 |
| REMARK B rmsd for angle sidechain atoms = | 6.020 | target = 2.5 |

REMARK wa = 2.10182
REMARK rweight = 6.273956E−02
REMARK target = mlf steps = 10
REMARK sg = P6(4) a = 108.074 b = 108.074 c = 84.385 alpha = 90 beta = 90 gamma = 120
REMARK parameter file 1: MSI_CNX_TOPPAR: protein_rep.param
REMARK parameter file 2: ../toppar/het.par
REMARK parameter file 3: MSI_CNX_TOPPAR: water_rep.param
REMARK molecular structure file: ../generate/generate.psf
REMARK input coordinates: minimize.pdb
REMARK reflection file = ../pdhk17.cv
REMARK ncs = none
REMARK B-correction resolution: 6.0 - 2.35
REMARK initial B-factor correction applied to fobs:

| | | | | | | |
|---|---|---|---|---|---|---|
| REMARK | B11 = | −4.378 | B22 = | −4.378 | B33 = | 8.757 |
| REMARK | B12 = | −5.201 | B13 = | 0.000 | B23 = | 0.000 |

REMARK B-factor correction applied to coordinate array B: -0.009
REMARK bulk solvent: (Mask) density level = 0.383445 e/A\hat{ }3, B-factor = 68.4975 A\hat{ }2
REMARK reflections with |Fobs|/sigma_F < 0.0 rejected
REMARK reflections with |Fobs| > 10000 * rms(Fobs) rejected

| | | |
|---|---|---|
| REMARK theoretical total number of refl. in resol. range: | 23429 | (100.0%) |
| REMARK number of unobserved reflections (no entry or \|F\| = 0): | 560 | (2.4%) |
| REMARK number of reflections rejected: | 0 | (0.0%) |
| REMARK total number of reflections used: | 22869 | (97.6%) |
| REMARK number of reflections in working set: | 21762 | (92.9%) |
| REMARK number of reflections in test set: | 1107 | (4.7%) |
| REMARK FILENAME = “bindividual.pdb” | | |
| REMARK DATE: Apr. 03, 2001 | | |
| 17:54:01 created by user: knoechel | | |
| REMARK Written by CNX VERSION: 2000 | | |

| | | | | | | | | | | | | |
|---|---|---|---|---|---|---|---|---|---|---|---|---|
| ATOM | 1 | C | GLY | A | 14 | 60.607 | 42.192 | 101.416 | 1.00 | 61.87 | A | C |
| ATOM | 2 | O | GLY | A | 14 | 59.849 | 42.296 | 102.380 | 1.00 | 59.30 | A | O |
| ATOM | 3 | N | GLY | A | 14 | 62.772 | 41.286 | 100.679 | 1.00 | 60.72 | A | N |
| ATOM | 4 | CA | GLY | A | 14 | 61.439 | 40.943 | 101.223 | 1.00 | 59.81 | A | C |
| ATOM | 5 | N | SER | A | 15 | 60.737 | 43.137 | 100.489 | 1.00 | 64.54 | A | N |
| ATOM | 6 | CA | SER | A | 15 | 59.996 | 44.396 | 100.573 | 1.00 | 68.33 | A | C |
| ATOM | 7 | CB | SER | A | 15 | 60.982 | 45.568 | 100.691 | 1.00 | 71.35 | A | C |
| ATOM | 8 | OG | SER | A | 15 | 61.947 | 45.537 | 99.650 | 1.00 | 77.56 | A | O |
| ATOM | 9 | C | SER | A | 15 | 59.018 | 44.652 | 99.419 | 1.00 | 67.19 | A | C |
| ATOM | 10 | O | SER | A | 15 | 58.491 | 45.757 | 99.276 | 1.00 | 67.80 | A | O |
| ATOM | 11 | N | ALA | A | 16 | 58.767 | 43.633 | 98.606 | 1.00 | 66.05 | A | N |
| ATOM | 12 | CA | ALA | A | 16 | 57.846 | 43.776 | 97.481 | 1.00 | 64.32 | A | C |
| ATOM | 13 | CB | ALA | A | 16 | 57.659 | 42.436 | 96.780 | 1.00 | 64.58 | A | C |
| ATOM | 14 | C | ALA | A | 16 | 56.487 | 44.331 | 97.904 | 1.00 | 63.22 | A | C |
| ATOM | 15 | O | ALA | A | 16 | 55.989 | 45.280 | 97.301 | 1.00 | 61.59 | A | O |
| ATOM | 16 | N | PRO | A | 17 | 55.868 | 43.747 | 98.944 | 1.00 | 62.31 | A | N |
| ATOM | 17 | CD | PRO | A | 17 | 56.315 | 42.590 | 99.738 | 1.00 | 62.46 | A | C |
| ATOM | 18 | CA | PRO | A | 17 | 54.561 | 44.221 | 99.409 | 1.00 | 62.27 | A | C |
| ATOM | 19 | CB | PRO | A | 17 | 54.291 | 43.355 | 100.634 | 1.00 | 62.00 | A | C |
| ATOM | 20 | CG | PRO | A | 17 | 55.019 | 42.083 | 100.314 | 1.00 | 62.48 | A | C |
| ATOM | 21 | C | PRO | A | 17 | 54.580 | 45.700 | 99.759 | 1.00 | 65.52 | A | C |
| ATOM | 22 | O | PRO | A | 17 | 53.568 | 46.394 | 99.629 | 1.00 | 65.51 | A | O |
| ATOM | 23 | N | LYS | A | 18 | 55.740 | 46.172 | 100.212 | 1.00 | 68.91 | A | N |
| ATOM | 24 | CA | LYS | A | 18 | 55.913 | 47.569 | 100.592 | 1.00 | 69.88 | A | C |
| ATOM | 25 | CB | LYS | A | 18 | 57.228 | 47.743 | 101.362 | 1.00 | 71.87 | A | C |
| ATOM | 26 | CG | LYS | A | 18 | 57.507 | 49.173 | 101.807 | 1.00 | 74.97 | A | C |
| ATOM | 27 | CD | LYS | A | 18 | 58.938 | 49.334 | 102.300 | 1.00 | 79.79 | A | C |
| ATOM | 28 | CE | LYS | A | 18 | 59.267 | 50.796 | 102.562 | 1.00 | 81.38 | A | C |
| ATOM | 29 | NZ | LYS | A | 18 | 60.712 | 50.999 | 102.834 | 1.00 | 83.60 | A | N |
| ATOM | 30 | C | LYS | A | 18 | 55.917 | 48.465 | 99.354 | 1.00 | 69.54 | A | C |
| ATOM | 31 | O | LYS | A | 18 | 55.147 | 49.421 | 99.272 | 1.00 | 67.90 | A | O |
| ATOM | 32 | N | TYR | A | 19 | 56.789 | 48.154 | 98.398 | 1.00 | 69.97 | A | N |
| ATOM | 33 | CA | TYR | A | 19 | 56.885 | 48.936 | 97.171 | 1.00 | 73.31 | A | C |
| ATOM | 34 | CB | TYR | A | 19 | 57.859 | 48.290 | 96.193 | 1.00 | 76.92 | A | C |
| ATOM | 35 | CG | TYR | A | 19 | 59.290 | 48.249 | 96.659 | 1.00 | 82.12 | A | C |
| ATOM | 36 | CD1 | TYR | A | 19 | 59.829 | 49.285 | 97.422 | 1.00 | 82.99 | A | C |
| ATOM | 37 | CE1 | TYR | A | 19 | 61.169 | 49.280 | 97.794 | 1.00 | 86.57 | A | C |
| ATOM | 38 | CD2 | TYR | A | 19 | 60.129 | 47.204 | 96.281 | 1.00 | 83.77 | A | C |
| ATOM | 39 | CE2 | TYR | A | 19 | 61.471 | 47.190 | 96.644 | 1.00 | 86.68 | A | C |
| ATOM | 40 | CZ | TYR | A | 19 | 61.986 | 48.230 | 97.399 | 1.00 | 87.28 | A | C |
| ATOM | 41 | OH | TYR | A | 19 | 63.321 | 48.218 | 97.748 | 1.00 | 89.12 | A | O |
| ATOM | 42 | C | TYR | A | 19 | 55.527 | 49.042 | 96.496 | 1.00 | 72.78 | A | C |
| ATOM | 43 | O | TYR | A | 19 | 55.114 | 50.114 | 96.052 | 1.00 | 73.64 | A | O |
| ATOM | 44 | N | ILE | A | 20 | 54.848 | 47.909 | 96.411 | 1.00 | 71.05 | A | N |
| ATOM | 45 | CA | ILE | A | 20 | 53.539 | 47.845 | 95.800 | 1.00 | 70.67 | A | C |
| ATOM | 46 | CB | ILE | A | 20 | 53.044 | 46.391 | 95.746 | 1.00 | 67.63 | A | C |
| ATOM | 47 | CG2 | ILE | A | 20 | 51.646 | 46.328 | 95.176 | 1.00 | 65.25 | A | C |
| ATOM | 48 | CG1 | ILE | A | 20 | 54.002 | 45.569 | 94.885 | 1.00 | 66.62 | A | C |
| ATOM | 49 | CD1 | ILE | A | 20 | 53.742 | 44.084 | 94.940 | 1.00 | 63.63 | A | C |
| ATOM | 50 | C | ILE | A | 20 | 52.559 | 48.707 | 96.578 | 1.00 | 72.05 | A | C |
| ATOM | 51 | O | ILE | A | 20 | 51.715 | 49.365 | 95.988 | 1.00 | 72.70 | A | O |
| ATOM | 52 | N | GLU | A | 21 | 52.671 | 48.708 | 97.903 | 1.00 | 74.32 | A | N |
| ATOM | 53 | CA | GLU | A | 21 | 51.784 | 49.522 | 98.728 | 1.00 | 74.91 | A | C |
| ATOM | 54 | CB | GLU | A | 21 | 52.012 | 49.232 | 100.214 | 1.00 | 81.77 | A | C |
| ATOM | 55 | CG | GLU | A | 21 | 51.223 | 50.137 | 101.158 | 1.00 | 90.75 | A | C |
| ATOM | 56 | CD | GLU | A | 21 | 49.744 | 50.185 | 100.822 | 1.00 | 95.63 | A | C |
| ATOM | 57 | OE1 | GLU | A | 21 | 49.121 | 49.107 | 100.714 | 1.00 | 98.31 | A | O |
| ATOM | 58 | OE2 | GLU | A | 21 | 49.205 | 51.303 | 100.668 | 1.00 | 98.51 | A | O |
| ATOM | 59 | C | GLU | A | 21 | 52.054 | 50.994 | 98.449 | 1.00 | 72.01 | A | C |
| ATOM | 60 | O | GLU | A | 21 | 51.141 | 51.815 | 98.455 | 1.00 | 72.91 | A | O |
| ATOM | 61 | N | HIS | A | 22 | 53.318 | 51.318 | 98.201 | 1.00 | 67.42 | A | N |
| ATOM | 62 | CA | HIS | A | 22 | 53.720 | 52.687 | 97.921 | 1.00 | 64.39 | A | C |
| ATOM | 63 | CB | HIS | A | 22 | 55.249 | 52.802 | 97.941 | 1.00 | 61.65 | A | C |
| ATOM | 64 | CG | HIS | A | 22 | 55.763 | 54.151 | 97.534 | 1.00 | 61.47 | A | C |
| ATOM | 65 | CD2 | HIS | A | 22 | 56.597 | 54.523 | 96.534 | 1.00 | 61.49 | A | C |
| ATOM | 66 | ND1 | HIS | A | 22 | 55.397 | 55.312 | 98.182 | 1.00 | 60.13 | A | N |
| ATOM | 67 | CE1 | HIS | A | 22 | 55.983 | 56.342 | 97.594 | 1.00 | 62.43 | A | C |
| ATOM | 68 | NE2 | HIS | A | 22 | 56.716 | 55.893 | 96.592 | 1.00 | 60.08 | A | N |
| ATOM | 69 | C | HIS | A | 22 | 53.183 | 53.156 | 96.572 | 1.00 | 62.60 | A | C |
| ATOM | 70 | O | HIS | A | 22 | 52.183 | 53.862 | 96.514 | 1.00 | 63.14 | A | O |
| ATOM | 71 | N | PHE | A | 23 | 53.854 | 52.751 | 95.495 | 1.00 | 61.56 | A | N |
| ATOM | 72 | CA | PHE | A | 23 | 53.472 | 53.134 | 94.137 | 1.00 | 60.99 | A | C |
| ATOM | 73 | CB | PHE | A | 23 | 54.343 | 52.400 | 93.121 | 1.00 | 57.63 | A | C |
| ATOM | 74 | CG | PHE | A | 23 | 55.797 | 52.736 | 93.224 | 1.00 | 55.11 | A | C |
| ATOM | 75 | CD1 | PHE | A | 23 | 56.675 | 51.898 | 93.911 | 1.00 | 55.59 | A | C |
| ATOM | 76 | CD2 | PHE | A | 23 | 56.292 | 53.904 | 92.650 | 1.00 | 52.60 | A | C |
| ATOM | 77 | CE1 | PHE | A | 23 | 58.026 | 52.223 | 94.023 | 1.00 | 52.29 | A | C |
| ATOM | 78 | CE2 | PHE | A | 23 | 57.636 | 54.237 | 92.757 | 1.00 | 51.93 | A | C |
| ATOM | 79 | CZ | PHE | A | 23 | 58.506 | 53.393 | 93.446 | 1.00 | 51.33 | A | C |
| ATOM | 80 | C | PHE | A | 23 | 51.999 | 52.919 | 93.797 | 1.00 | 60.66 | A | C |
| ATOM | 81 | O | PHE | A | 23 | 51.476 | 53.550 | 92.888 | 1.00 | 61.40 | A | O |
| ATOM | 82 | N | SER | A | 24 | 51.340 | 52.031 | 94.532 | 1.00 | 63.73 | A | N |
| ATOM | 83 | CA | SER | A | 24 | 49.926 | 51.735 | 94.328 | 1.00 | 66.74 | A | C |
| ATOM | 84 | CB | SER | A | 24 | 49.522 | 50.525 | 95.172 | 1.00 | 65.96 | A | C |
| ATOM | 85 | OG | SER | A | 24 | 48.127 | 50.494 | 95.421 | 1.00 | 69.56 | A | O |
| ATOM | 86 | C | SER | A | 24 | 49.061 | 52.927 | 94.721 | 1.00 | 70.40 | A | C |
| ATOM | 87 | O | SER | A | 24 | 47.879 | 52.987 | 94.378 | 1.00 | 69.67 | A | O |
| ATOM | 88 | N | LYS | A | 25 | 49.654 | 53.865 | 95.453 | 1.00 | 74.05 | A | N |
| ATOM | 89 | CA | LYS | A | 25 | 48.940 | 55.053 | 95.901 | 1.00 | 78.10 | A | C |
| ATOM | 90 | CB | LYS | A | 25 | 49.642 | 55.653 | 97.129 | 1.00 | 85.95 | A | C |
| ATOM | 91 | CG | LYS | A | 25 | 49.570 | 54.776 | 98.382 | 1.00 | 95.95 | A | C |
| ATOM | 92 | CD | LYS | A | 25 | 50.410 | 55.343 | 99.520 | 1.00 | 104.25 | A | C |
| ATOM | 93 | CE | LYS | A | 25 | 50.391 | 54.425 | 100.738 | 1.00 | 109.74 | A | C |
| ATOM | 94 | NZ | LYS | A | 25 | 51.376 | 54.853 | 101.775 | 1.00 | 113.26 | A | N |
| ATOM | 95 | C | LYS | A | 25 | 48.849 | 56.086 | 94.782 | 1.00 | 74.61 | A | C |
| ATOM | 96 | O | LYS | A | 25 | 47.995 | 56.965 | 94.810 | 1.00 | 74.47 | A | O |
| ATOM | 97 | N | PHE | A | 26 | 49.734 | 55.967 | 93.799 | 1.00 | 71.94 | A | N |
| ATOM | 98 | CA | PHE | A | 26 | 49.755 | 56.879 | 92.666 | 1.00 | 69.46 | A | C |
| ATOM | 99 | CB | PHE | A | 26 | 51.168 | 57.002 | 92.095 | 1.00 | 72.12 | A | C |
| ATOM | 100 | CG | PHE | A | 26 | 52.159 | 57.634 | 93.029 | 1.00 | 78.86 | A | C |
| ATOM | 101 | CD1 | PHE | A | 26 | 52.516 | 57.010 | 94.223 | 1.00 | 80.23 | A | C |
| ATOM | 102 | CD2 | PHE | A | 26 | 52.760 | 58.845 | 92.698 | 1.00 | 80.40 | A | C |
| ATOM | 103 | CE1 | PHE | A | 26 | 53.460 | 57.584 | 95.073 | 1.00 | 81.83 | A | C |
| ATOM | 104 | CE2 | PHE | A | 26 | 53.706 | 59.429 | 93.542 | 1.00 | 82.21 | A | C |
| ATOM | 105 | CZ | PHE | A | 26 | 54.057 | 58.796 | 94.731 | 1.00 | 82.94 | A | C |
| ATOM | 106 | C | PHE | A | 26 | 48.841 | 56.372 | 91.558 | 1.00 | 67.80 | A | C |
| ATOM | 107 | O | PHE | A | 26 | 48.589 | 55.171 | 91.446 | 1.00 | 67.17 | A | O |
| ATOM | 108 | N | SER | A | 27 | 48.344 | 57.295 | 90.742 | 1.00 | 63.25 | A | N |
| ATOM | 109 | CA | SER | A | 27 | 47.499 | 56.928 | 89.616 | 1.00 | 59.46 | A | C |
| ATOM | 110 | CB | SER | A | 27 | 46.348 | 57.921 | 89.447 | 1.00 | 59.22 | A | C |
| ATOM | 111 | OG | SER | A | 27 | 45.302 | 57.657 | 90.364 | 1.00 | 63.15 | A | O |
| ATOM | 112 | C | SER | A | 27 | 48.384 | 56.965 | 88.382 | 1.00 | 55.87 | A | C |
| ATOM | 113 | O | SER | A | 27 | 49.282 | 57.803 | 88.287 | 1.00 | 57.31 | A | O |
| ATOM | 114 | N | PRO | A | 28 | 48.181 | 56.031 | 87.435 | 1.00 | 52.12 | A | N |
| ATOM | 115 | CD | PRO | A | 28 | 47.435 | 54.761 | 87.492 | 1.00 | 47.55 | A | C |
| ATOM | 116 | CA | PRO | A | 28 | 49.040 | 56.093 | 86.246 | 1.00 | 51.08 | A | C |
| ATOM | 117 | CB | PRO | A | 28 | 48.752 | 54.767 | 85.531 | 1.00 | 50.54 | A | C |
| ATOM | 118 | CG | PRO | A | 28 | 47.414 | 54.349 | 86.050 | 1.00 | 48.49 | A | C |
| ATOM | 119 | C | PRO | A | 28 | 48.711 | 57.338 | 85.402 | 1.00 | 51.70 | A | C |
| ATOM | 120 | O | PRO | A | 28 | 47.591 | 57.847 | 85.451 | 1.00 | 48.48 | A | O |
| ATOM | 121 | N | SER | A | 29 | 49.693 | 57.828 | 84.649 | 1.00 | 53.29 | A | N |
| ATOM | 122 | CA | SER | A | 29 | 49.522 | 59.025 | 83.832 | 1.00 | 52.83 | A | C |
| ATOM | 123 | CB | SER | A | 29 | 50.781 | 59.898 | 83.900 | 1.00 | 55.52 | A | C |
| ATOM | 124 | OG | SER | A | 29 | 50.879 | 60.567 | 85.146 | 1.00 | 63.86 | A | O |
| ATOM | 125 | C | SER | A | 29 | 49.208 | 58.730 | 82.377 | 1.00 | 51.66 | A | C |
| ATOM | 126 | O | SER | A | 29 | 50.109 | 58.464 | 81.582 | 1.00 | 51.36 | A | O |
| ATOM | 127 | N | PRO | A | 30 | 47.925 | 58.792 | 82.003 | 1.00 | 49.94 | A | N |
| ATOM | 128 | CD | PRO | A | 30 | 46.733 | 59.052 | 82.829 | 1.00 | 50.02 | A | C |
| ATOM | 129 | CA | PRO | A | 30 | 47.562 | 58.519 | 80.609 | 1.00 | 49.28 | A | C |
| ATOM | 130 | CB | PRO | A | 30 | 46.026 | 58.540 | 80.628 | 1.00 | 47.59 | A | C |
| ATOM | 131 | CG | PRO | A | 30 | 45.693 | 59.415 | 81.786 | 1.00 | 51.13 | A | C |
| ATOM | 132 | C | PRO | A | 30 | 48.172 | 59.550 | 79.670 | 1.00 | 49.70 | A | C |
| ATOM | 133 | O | PRO | A | 30 | 47.996 | 60.746 | 79.859 | 1.00 | 52.23 | A | O |
| ATOM | 134 | N | LEU | A | 31 | 48.908 | 59.068 | 78.669 | 1.00 | 49.19 | A | N |
| ATOM | 135 | CA | LEU | A | 31 | 49.573 | 59.918 | 77.692 | 1.00 | 47.39 | A | C |
| ATOM | 136 | CB | LEU | A | 31 | 50.972 | 59.375 | 77.400 | 1.00 | 44.62 | A | C |
| ATOM | 137 | CG | LEU | A | 31 | 51.896 | 59.064 | 78.588 | 1.00 | 48.44 | A | C |
| ATOM | 138 | CD1 | LEU | A | 31 | 53.110 | 58.270 | 78.108 | 1.00 | 49.36 | A | C |
| ATOM | 139 | CD2 | LEU | A | 31 | 52.337 | 60.354 | 79.254 | 1.00 | 45.40 | A | C |
| ATOM | 140 | C | LEU | A | 31 | 48.778 | 59.974 | 76.389 | 1.00 | 51.19 | A | C |
| ATOM | 141 | O | LEU | A | 31 | 48.101 | 59.014 | 76.018 | 1.00 | 52.48 | A | O |
| ATOM | 142 | N | SER | A | 32 | 48.874 | 61.099 | 75.688 | 1.00 | 53.11 | A | N |
| ATOM | 143 | CA | SER | A | 32 | 48.167 | 61.269 | 74.425 | 1.00 | 52.19 | A | C |
| ATOM | 144 | CB | SER | A | 32 | 47.739 | 62.718 | 74.237 | 1.00 | 49.93 | A | C |
| ATOM | 145 | OG | SER | A | 32 | 48.781 | 63.439 | 73.610 | 1.00 | 54.22 | A | O |
| ATOM | 146 | C | SER | A | 32 | 49.134 | 60.893 | 73.318 | 1.00 | 53.12 | A | C |
| ATOM | 147 | O | SER | A | 32 | 50.340 | 60.825 | 73.536 | 1.00 | 55.03 | A | O |
| ATOM | 148 | N | MET | A | 33 | 48.614 | 60.669 | 72.121 | 1.00 | 54.56 | A | N |
| ATOM | 149 | CA | MET | A | 33 | 49.457 | 60.286 | 71.009 | 1.00 | 55.95 | A | C |
| ATOM | 150 | CB | MET | A | 33 | 48.600 | 59.700 | 69.889 | 1.00 | 58.08 | A | C |
| ATOM | 151 | CG | MET | A | 33 | 49.387 | 58.988 | 68.811 | 1.00 | 57.99 | A | C |
| ATOM | 152 | SD | MET | A | 33 | 48.710 | 57.344 | 68.490 | 1.00 | 62.97 | A | S |
| ATOM | 153 | CE | MET | A | 33 | 50.191 | 56.442 | 68.201 | 1.00 | 63.20 | A | C |
| ATOM | 154 | C | MET | A | 33 | 50.299 | 61.441 | 70.479 | 1.00 | 58.01 | A | C |
| ATOM | 155 | O | MET | A | 33 | 51.279 | 61.216 | 69.771 | 1.00 | 57.05 | A | O |
| ATOM | 156 | N | LYS | A | 34 | 49.933 | 62.675 | 70.811 | 1.00 | 59.78 | A | N |
| ATOM | 157 | CA | LYS | A | 34 | 50.730 | 63.799 | 70.334 | 1.00 | 63.30 | A | C |
| ATOM | 158 | CB | LYS | A | 34 | 49.903 | 65.092 | 70.243 | 1.00 | 65.66 | A | C |
| ATOM | 159 | CG | LYS | A | 34 | 50.354 | 65.983 | 69.080 | 1.00 | 68.02 | A | C |
| ATOM | 160 | CD | LYS | A | 34 | 49.421 | 67.150 | 68.806 | 1.00 | 73.03 | A | C |
| ATOM | 161 | CE | LYS | A | 34 | 49.653 | 68.296 | 69.778 | 1.00 | 76.83 | A | C |
| ATOM | 162 | NZ | LYS | A | 34 | 48.504 | 68.476 | 70.706 | 1.00 | 81.88 | A | N |
| ATOM | 163 | C | LYS | A | 34 | 51.937 | 64.007 | 71.240 | 1.00 | 61.89 | A | C |
| ATOM | 164 | O | LYS | A | 34 | 52.988 | 64.446 | 70.781 | 1.00 | 62.83 | A | O |
| ATOM | 165 | N | GLN | A | 35 | 51.790 | 63.692 | 72.524 | 1.00 | 62.46 | A | N |
| ATOM | 166 | CA | GLN | A | 35 | 52.905 | 63.832 | 73.457 | 1.00 | 62.74 | A | C |
| ATOM | 167 | CB | GLN | A | 35 | 52.450 | 63.558 | 74.880 | 1.00 | 59.46 | A | C |
| ATOM | 168 | CG | GLN | A | 35 | 51.045 | 64.018 | 75.161 | 1.00 | 61.92 | A | C |
| ATOM | 169 | CD | GLN | A | 35 | 50.705 | 63.967 | 76.631 | 1.00 | 60.96 | A | C |
| ATOM | 170 | OE1 | GLN | A | 35 | 49.685 | 63.405 | 77.030 | 1.00 | 62.27 | A | O |
| ATOM | 171 | NE2 | GLN | A | 35 | 51.559 | 64.563 | 77.450 | 1.00 | 60.57 | A | N |
| ATOM | 172 | C | GLN | A | 35 | 53.974 | 62.814 | 73.063 | 1.00 | 64.47 | A | C |
| ATOM | 173 | O | GLN | A | 35 | 55.165 | 63.013 | 73.311 | 1.00 | 67.07 | A | O |
| ATOM | 174 | N | PHE | A | 36 | 53.528 | 61.719 | 72.450 | 1.00 | 63.72 | A | N |
| ATOM | 175 | CA | PHE | A | 36 | 54.409 | 60.643 | 71.987 | 1.00 | 64.08 | A | C |
| ATOM | 176 | CB | PHE | A | 36 | 53.584 | 59.408 | 71.630 | 1.00 | 59.39 | A | C |
| ATOM | 177 | CG | PHE | A | 36 | 53.243 | 58.516 | 72.795 | 1.00 | 57.20 | A | C |
| ATOM | 178 | CD1 | PHE | A | 36 | 51.918 | 58.154 | 73.048 | 1.00 | 54.15 | A | C |
| ATOM | 179 | CD2 | PHE | A | 36 | 54.243 | 57.990 | 73.602 | 1.00 | 54.31 | A | C |
| ATOM | 180 | CE1 | PHE | A | 36 | 51.599 | 57.279 | 74.090 | 1.00 | 52.36 | A | C |
| ATOM | 181 | CE2 | PHE | A | 36 | 53.935 | 57.113 | 74.648 | 1.00 | 54.10 | A | C |
| ATOM | 182 | CZ | PHE | A | 36 | 52.612 | 56.757 | 74.892 | 1.00 | 53.43 | A | C |
| ATOM | 183 | C | PHE | A | 36 | 55.181 | 61.089 | 70.742 | 1.00 | 68.46 | A | C |
| ATOM | 184 | O | PHE | A | 36 | 56.307 | 60.641 | 70.506 | 1.00 | 67.45 | A | O |
| ATOM | 185 | N | LEU | A | 37 | 54.538 | 61.945 | 69.948 | 1.00 | 75.98 | A | N |
| ATOM | 186 | CA | LEU | A | 37 | 55.105 | 62.503 | 68.731 | 1.00 | 81.86 | A | C |
| ATOM | 187 | CB | LEU | A | 37 | 54.009 | 63.176 | 67.909 | 1.00 | 82.74 | A | C |
| ATOM | 188 | CG | LEU | A | 37 | 53.182 | 62.301 | 67.007 | 1.00 | 83.56 | A | C |
| ATOM | 189 | CD1 | LEU | A | 37 | 52.096 | 63.176 | 66.418 | 1.00 | 85.01 | A | C |
| ATOM | 190 | CD2 | LEU | A | 37 | 54.079 | 61.722 | 65.940 | 1.00 | 82.78 | A | C |
| ATOM | 191 | C | LEU | A | 37 | 56.156 | 63.511 | 68.935 | 1.00 | 85.01 | A | C |
| ATOM | 192 | O | LEU | A | 37 | 56.877 | 63.882 | 68.035 | 1.00 | 86.66 | A | O |
| ATOM | 193 | N | ASP | A | 38 | 56.114 | 64.096 | 70.098 | 1.00 | 89.60 | A | N |
| ATOM | 194 | CA | ASP | A | 38 | 57.071 | 65.119 | 70.339 | 1.00 | 92.95 | A | C |
| ATOM | 195 | CB | ASP | A | 38 | 56.580 | 65.988 | 71.478 | 1.00 | 99.12 | A | C |
| ATOM | 196 | CG | ASP | A | 38 | 55.282 | 66.676 | 71.106 | 1.00 | 104.85 | A | C |
| ATOM | 197 | OD1 | ASP | A | 38 | 55.088 | 66.869 | 69.890 | 1.00 | 106.77 | A | O |
| ATOM | 198 | OD2 | ASP | A | 38 | 54.475 | 67.004 | 71.994 | 1.00 | 109.04 | A | O |
| ATOM | 199 | C | ASP | A | 38 | 58.422 | 64.520 | 70.503 | 1.00 | 92.08 | A | C |
| ATOM | 200 | O | ASP | A | 38 | 59.380 | 64.995 | 69.929 | 1.00 | 93.01 | A | O |
| ATOM | 201 | N | PHE | A | 39 | 58.510 | 63.415 | 71.208 | 1.00 | 89.97 | A | N |
| ATOM | 202 | CA | PHE | A | 39 | 59.812 | 62.793 | 71.274 | 1.00 | 88.81 | A | C |
| ATOM | 203 | CB | PHE | A | 39 | 59.774 | 61.580 | 72.187 | 1.00 | 88.83 | A | C |
| ATOM | 204 | CG | PHE | A | 39 | 60.103 | 61.914 | 73.588 | 1.00 | 87.43 | A | C |
| ATOM | 205 | CD1 | PHE | A | 39 | 59.706 | 63.138 | 74.120 | 1.00 | 86.89 | A | C |
| ATOM | 206 | CD2 | PHE | A | 39 | 60.847 | 61.045 | 74.366 | 1.00 | 86.48 | A | C |
| ATOM | 207 | CE1 | PHE | A | 39 | 60.054 | 63.491 | 75.402 | 1.00 | 86.64 | A | C |
| ATOM | 208 | CE2 | PHE | A | 39 | 61.202 | 61.388 | 75.655 | 1.00 | 85.77 | A | C |
| ATOM | 209 | CZ | PHE | A | 39 | 60.807 | 62.617 | 76.178 | 1.00 | 86.83 | A | C |
| ATOM | 210 | C | PHE | A | 39 | 60.219 | 62.391 | 69.859 | 1.00 | 87.73 | A | C |
| ATOM | 211 | O | PHE | A | 39 | 59.430 | 62.540 | 68.927 | 1.00 | 87.96 | A | O |
| ATOM | 212 | N | SER | A | 42 | 66.023 | 61.267 | 67.822 | 1.00 | 105.11 | A | N |
| ATOM | 213 | CA | SER | A | 42 | 66.846 | 60.525 | 68.766 | 1.00 | 105.38 | A | C |
| ATOM | 214 | CB | SER | A | 42 | 67.835 | 59.618 | 68.025 | 1.00 | 104.79 | A | C |
| ATOM | 215 | OG | SER | A | 42 | 68.668 | 58.920 | 68.941 | 1.00 | 101.59 | A | O |
| ATOM | 216 | C | SER | A | 42 | 67.615 | 61.458 | 69.691 | 1.00 | 106.30 | A | C |
| ATOM | 217 | O | SER | A | 42 | 68.592 | 62.092 | 69.286 | 1.00 | 106.94 | A | O |
| ATOM | 218 | N | ASN | A | 43 | 67.143 | 61.531 | 70.930 | 1.00 | 105.94 | A | N |
| ATOM | 219 | CA | ASN | A | 43 | 67.727 | 62.339 | 71.994 | 1.00 | 105.46 | A | C |
| ATOM | 220 | CB | ASN | A | 43 | 69.193 | 62.691 | 71.720 | 1.00 | 107.78 | A | C |
| ATOM | 221 | CG | ASN | A | 43 | 69.908 | 63.200 | 72.964 | 1.00 | 109.38 | A | C |
| ATOM | 222 | OD1 | ASN | A | 43 | 69.334 | 63.229 | 74.055 | 1.00 | 109.82 | A | O |
| ATOM | 223 | ND2 | ASN | A | 43 | 71.165 | 63.598 | 72.805 | 1.00 | 110.45 | A | N |
| ATOM | 224 | C | ASN | A | 43 | 66.965 | 63.605 | 72.350 | 1.00 | 103.96 | A | C |
| ATOM | 225 | O | ASN | A | 43 | 67.545 | 64.692 | 72.449 | 1.00 | 103.44 | A | O |
| ATOM | 226 | N | ALA | A | 44 | 65.654 | 63.458 | 72.520 | 1.00 | 101.37 | A | N |
| ATOM | 227 | CA | ALA | A | 44 | 64.821 | 64.557 | 72.978 | 1.00 | 97.67 | A | C |
| ATOM | 228 | CB | ALA | A | 44 | 63.384 | 64.368 | 72.538 | 1.00 | 99.15 | A | C |
| ATOM | 229 | C | ALA | A | 44 | 64.990 | 64.212 | 74.451 | 1.00 | 95.78 | A | C |
| ATOM | 230 | O | ALA | A | 44 | 64.058 | 64.250 | 75.252 | 1.00 | 95.28 | A | O |
| ATOM | 231 | N | CYS | A | 45 | 66.229 | 63.820 | 74.748 | 1.00 | 93.20 | A | N |
| ATOM | 232 | CA | CYS | A | 45 | 66.703 | 63.403 | 76.052 | 1.00 | 90.05 | A | C |
| ATOM | 233 | CB | CYS | A | 45 | 66.606 | 64.546 | 77.057 | 1.00 | 90.72 | A | C |
| ATOM | 234 | SG | CYS | A | 45 | 68.150 | 65.453 | 77.151 | 1.00 | 95.49 | A | S |
| ATOM | 235 | C | CYS | A | 45 | 66.048 | 62.163 | 76.619 | 1.00 | 86.48 | A | C |
| ATOM | 236 | O | CYS | A | 45 | 64.886 | 62.182 | 77.020 | 1.00 | 85.53 | A | O |
| ATOM | 237 | N | GLU | A | 46 | 66.803 | 61.070 | 76.631 | 1.00 | 82.69 | A | N |
| ATOM | 238 | CA | GLU | A | 46 | 66.294 | 59.840 | 77.202 | 1.00 | 80.90 | A | C |
| ATOM | 239 | CB | GLU | A | 46 | 67.074 | 58.627 | 76.712 | 1.00 | 78.81 | A | C |
| ATOM | 240 | CG | GLU | A | 46 | 68.552 | 58.816 | 76.608 | 1.00 | 76.52 | A | C |
| ATOM | 241 | CD | GLU | A | 46 | 69.222 | 57.584 | 76.039 | 1.00 | 74.74 | A | C |
| ATOM | 242 | OE1 | GLU | A | 46 | 69.081 | 56.508 | 76.659 | 1.00 | 70.81 | A | O |
| ATOM | 243 | OE2 | GLU | A | 46 | 69.878 | 57.690 | 74.978 | 1.00 | 72.72 | A | O |
| ATOM | 244 | C | GLU | A | 46 | 66.453 | 60.000 | 78.691 | 1.00 | 80.63 | A | C |
| ATOM | 245 | O | GLU | A | 46 | 65.968 | 59.187 | 79.474 | 1.00 | 80.47 | A | O |
| ATOM | 246 | N | LYS | A | 47 | 67.155 | 61.060 | 79.075 | 1.00 | 81.69 | A | N |
| ATOM | 247 | CA | LYS | A | 47 | 67.333 | 61.361 | 80.485 | 1.00 | 82.23 | A | C |
| ATOM | 248 | CB | LYS | A | 47 | 68.237 | 62.585 | 80.678 | 1.00 | 88.67 | A | C |
| ATOM | 249 | CG | LYS | A | 47 | 69.635 | 62.455 | 80.087 | 1.00 | 97.17 | A | C |
| ATOM | 250 | CD | LYS | A | 47 | 70.484 | 63.682 | 80.410 | 1.00 | 104.57 | A | C |
| ATOM | 251 | CE | LYS | A | 47 | 69.843 | 64.959 | 79.884 | 1.00 | 109.34 | A | C |
| ATOM | 252 | NZ | LYS | A | 47 | 70.650 | 66.168 | 80.199 | 1.00 | 112.65 | A | N |
| ATOM | 253 | C | LYS | A | 47 | 65.911 | 61.699 | 80.909 | 1.00 | 77.81 | A | C |
| ATOM | 254 | O | LYS | A | 47 | 65.385 | 61.152 | 81.880 | 1.00 | 76.04 | A | O |
| ATOM | 255 | N | THR | A | 48 | 65.292 | 62.591 | 80.137 | 1.00 | 73.98 | A | N |
| ATOM | 256 | CA | THR | A | 48 | 63.924 | 63.030 | 80.377 | 1.00 | 70.79 | A | C |
| ATOM | 257 | CB | THR | A | 48 | 63.442 | 63.979 | 79.266 | 1.00 | 71.14 | A | C |
| ATOM | 258 | OG1 | THR | A | 48 | 64.303 | 65.125 | 79.207 | 1.00 | 71.33 | A | O |
| ATOM | 259 | CG2 | THR | A | 48 | 62.014 | 64.427 | 79.534 | 1.00 | 68.85 | A | C |
| ATOM | 260 | C | THR | A | 48 | 62.968 | 61.840 | 80.449 | 1.00 | 68.92 | A | C |
| ATOM | 261 | O | THR | A | 48 | 62.007 | 61.861 | 81.214 | 1.00 | 68.06 | A | O |
| ATOM | 262 | N | SER | A | 49 | 63.226 | 60.810 | 79.647 | 1.00 | 67.20 | A | N |
| ATOM | 263 | CA | SER | A | 49 | 62.384 | 59.619 | 79.659 | 1.00 | 66.51 | A | C |
| ATOM | 264 | CB | SER | A | 49 | 62.577 | 58.803 | 78.382 | 1.00 | 66.58 | A | C |
| ATOM | 265 | OG | SER | A | 49 | 61.846 | 57.589 | 78.459 | 1.00 | 63.83 | A | O |
| ATOM | 266 | C | SER | A | 49 | 62.716 | 58.742 | 80.859 | 1.00 | 67.56 | A | C |
| ATOM | 267 | O | SER | A | 49 | 61.822 | 58.348 | 81.613 | 1.00 | 69.59 | A | O |
| ATOM | 268 | N | PHE | A | 50 | 64.004 | 58.434 | 81.020 | 1.00 | 66.23 | A | N |
| ATOM | 269 | CA | PHE | A | 50 | 64.483 | 57.608 | 82.124 | 1.00 | 63.38 | A | C |
| ATOM | 270 | CB | PHE | A | 50 | 66.005 | 57.719 | 82.240 | 1.00 | 66.30 | A | C |
| ATOM | 271 | CG | PHE | A | 50 | 66.572 | 57.051 | 83.464 | 1.00 | 68.76 | A | C |
| ATOM | 272 | CD1 | PHE | A | 50 | 66.468 | 55.671 | 83.631 | 1.00 | 69.53 | A | C |
| ATOM | 273 | CD2 | PHE | A | 50 | 67.204 | 57.799 | 84.454 | 1.00 | 68.40 | A | C |
| ATOM | 274 | CE1 | PHE | A | 50 | 66.985 | 55.041 | 84.764 | 1.00 | 69.81 | A | C |
| ATOM | 275 | CE2 | PHE | A | 50 | 67.726 | 57.179 | 85.594 | 1.00 | 70.98 | A | C |
| ATOM | 276 | CZ | PHE | A | 50 | 67.616 | 55.796 | 85.749 | 1.00 | 70.00 | A | C |
| ATOM | 277 | C | PHE | A | 50 | 63.845 | 58.025 | 83.441 | 1.00 | 61.86 | A | C |
| ATOM | 278 | O | PHE | A | 50 | 63.262 | 57.202 | 84.152 | 1.00 | 61.19 | A | O |
| ATOM | 279 | N | THR | A | 51 | 63.953 | 59.307 | 83.765 | 1.00 | 61.20 | A | N |
| ATOM | 280 | CA | THR | A | 51 | 63.385 | 59.810 | 85.005 | 1.00 | 63.11 | A | C |
| ATOM | 281 | CB | THR | A | 51 | 63.719 | 61.302 | 85.203 | 1.00 | 63.09 | A | C |
| ATOM | 282 | OG1 | THR | A | 51 | 63.485 | 62.015 | 83.984 | 1.00 | 66.43 | A | O |
| ATOM | 283 | CG2 | THR | A | 51 | 65.171 | 61.469 | 85.586 | 1.00 | 65.28 | A | C |
| ATOM | 284 | C | THR | A | 51 | 61.875 | 59.604 | 85.041 | 1.00 | 63.12 | A | C |
| ATOM | 285 | O | THR | A | 51 | 61.317 | 59.287 | 86.093 | 1.00 | 63.20 | A | O |
| ATOM | 286 | N | PHE | A | 52 | 61.212 | 59.775 | 83.897 | 1.00 | 63.37 | A | N |
| ATOM | 287 | CA | PHE | A | 52 | 59.763 | 59.584 | 83.837 | 1.00 | 61.62 | A | C |
| ATOM | 288 | CB | PHE | A | 52 | 59.187 | 60.054 | 82.501 | 1.00 | 63.66 | A | C |
| ATOM | 289 | CG | PHE | A | 52 | 57.741 | 59.662 | 82.304 | 1.00 | 66.60 | A | C |
| ATOM | 290 | CD1 | PHE | A | 52 | 56.742 | 60.227 | 83.089 | 1.00 | 65.72 | A | C |
| ATOM | 291 | CD2 | PHE | A | 52 | 57.385 | 58.686 | 81.372 | 1.00 | 66.38 | A | C |
| ATOM | 292 | CE1 | PHE | A | 52 | 55.415 | 59.828 | 82.954 | 1.00 | 66.87 | A | C |
| ATOM | 293 | CE2 | PHE | A | 52 | 56.057 | 58.281 | 81.229 | 1.00 | 66.21 | A | C |
| ATOM | 294 | CZ | PHE | A | 52 | 55.073 | 58.853 | 82.022 | 1.00 | 67.31 | A | C |
| ATOM | 295 | C | PHE | A | 52 | 59.383 | 58.122 | 84.021 | 1.00 | 60.43 | A | C |
| ATOM | 296 | O | PHE | A | 52 | 58.546 | 57.792 | 84.864 | 1.00 | 60.33 | A | O |
| ATOM | 297 | N | LEU | A | 53 | 59.993 | 57.253 | 83.215 | 1.00 | 59.42 | A | N |
| ATOM | 298 | CA | LEU | A | 53 | 59.710 | 55.820 | 83.268 | 1.00 | 61.82 | A | C |
| ATOM | 299 | CB | LEU | A | 53 | 60.443 | 55.100 | 82.129 | 1.00 | 58.95 | A | C |
| ATOM | 300 | CG | LEU | A | 53 | 59.888 | 55.433 | 80.739 | 1.00 | 58.99 | A | C |
| ATOM | 301 | CD1 | LEU | A | 53 | 60.698 | 54.739 | 79.652 | 1.00 | 55.89 | A | C |
| ATOM | 302 | CD2 | LEU | A | 53 | 58.420 | 55.011 | 80.680 | 1.00 | 56.38 | A | C |
| ATOM | 303 | C | LEU | A | 53 | 60.058 | 55.183 | 84.605 | 1.00 | 64.60 | A | C |
| ATOM | 304 | O | LEU | A | 53 | 59.515 | 54.138 | 84.958 | 1.00 | 65.32 | A | O |
| ATOM | 305 | N | ARG | A | 54 | 60.969 | 55.816 | 85.341 | 1.00 | 70.36 | A | N |
| ATOM | 306 | CA | ARG | A | 54 | 61.392 | 55.331 | 86.655 | 1.00 | 73.56 | A | C |
| ATOM | 307 | CB | ARG | A | 54 | 62.496 | 56.222 | 87.220 | 1.00 | 80.56 | A | C |
| ATOM | 308 | CG | ARG | A | 54 | 63.889 | 55.895 | 86.749 | 1.00 | 87.94 | A | C |
| ATOM | 309 | CD | ARG | A | 54 | 64.843 | 57.028 | 87.102 | 1.00 | 95.18 | A | C |
| ATOM | 310 | NE | ARG | A | 54 | 64.666 | 57.525 | 88.465 | 1.00 | 99.94 | A | N |
| ATOM | 311 | CZ | ARG | A | 54 | 65.461 | 58.426 | 89.038 | 1.00 | 102.65 | A | C |
| ATOM | 312 | NH1 | ARG | A | 54 | 66.491 | 58.925 | 88.365 | 1.00 | 103.15 | A | N |
| ATOM | 313 | NH2 | ARG | A | 54 | 65.223 | 58.836 | 90.277 | 1.00 | 103.34 | A | N |
| ATOM | 314 | C | ARG | A | 54 | 60.228 | 55.321 | 87.638 | 1.00 | 72.46 | A | C |
| ATOM | 315 | O | ARG | A | 54 | 60.135 | 54.438 | 88.488 | 1.00 | 71.82 | A | O |
| ATOM | 316 | N | GLN | A | 55 | 59.349 | 56.311 | 87.527 | 1.00 | 69.73 | A | N |
| ATOM | 317 | CA | GLN | A | 55 | 58.196 | 56.401 | 88.417 | 1.00 | 68.47 | A | C |
| ATOM | 318 | CB | GLN | A | 55 | 57.935 | 57.873 | 88.789 | 1.00 | 70.08 | A | C |
| ATOM | 319 | CG | GLN | A | 55 | 56.571 | 58.145 | 89.422 | 1.00 | 71.11 | A | C |
| ATOM | 320 | CD | GLN | A | 55 | 56.509 | 57.802 | 90.896 | 1.00 | 72.89 | A | C |
| ATOM | 321 | OE1 | GLN | A | 55 | 57.275 | 56.976 | 91.387 | 1.00 | 75.10 | A | O |
| ATOM | 322 | NE2 | GLN | A | 55 | 55.577 | 58.427 | 91.609 | 1.00 | 73.82 | A | N |
| ATOM | 323 | C | GLN | A | 55 | 56.940 | 55.790 | 87.789 | 1.00 | 65.27 | A | C |
| ATOM | 324 | O | GLN | A | 55 | 56.180 | 55.092 | 88.460 | 1.00 | 64.28 | A | O |
| ATOM | 325 | N | GLU | A | 56 | 56.741 | 56.035 | 86.497 | 1.00 | 63.28 | A | N |
| ATOM | 326 | CA | GLU | A | 56 | 55.559 | 55.544 | 85.791 | 1.00 | 60.51 | A | C |
| ATOM | 327 | CB | GLU | A | 56 | 55.533 | 56.121 | 84.370 | 1.00 | 61.02 | A | C |
| ATOM | 328 | CG | GLU | A | 56 | 54.151 | 56.131 | 83.734 | 1.00 | 60.73 | A | C |
| ATOM | 329 | CD | GLU | A | 56 | 53.074 | 56.595 | 84.701 | 1.00 | 60.87 | A | C |
| ATOM | 330 | OE1 | GLU | A | 56 | 53.363 | 57.480 | 85.534 | 1.00 | 63.57 | A | O |
| ATOM | 331 | OE2 | GLU | A | 56 | 51.937 | 56.081 | 84.626 | 1.00 | 58.69 | A | O |
| ATOM | 332 | C | GLU | A | 56 | 55.430 | 54.020 | 85.739 | 1.00 | 57.54 | A | C |
| ATOM | 333 | O | GLU | A | 56 | 54.395 | 53.464 | 86.116 | 1.00 | 55.43 | A | O |
| ATOM | 334 | N | LEU | A | 57 | 56.469 | 53.340 | 85.268 | 1.00 | 53.76 | A | N |
| ATOM | 335 | CA | LEU | A | 57 | 56.417 | 51.889 | 85.195 | 1.00 | 52.79 | A | C |
| ATOM | 336 | CB | LEU | A | 57 | 57.737 | 51.348 | 84.653 | 1.00 | 54.81 | A | C |
| ATOM | 337 | CG | LEU | A | 57 | 57.852 | 51.553 | 83.141 | 1.00 | 54.66 | A | C |
| ATOM | 338 | CD1 | LEU | A | 57 | 59.265 | 51.263 | 82.663 | 1.00 | 53.71 | A | C |
| ATOM | 339 | CD2 | LEU | A | 57 | 56.836 | 50.642 | 82.447 | 1.00 | 55.97 | A | C |
| ATOM | 340 | C | LEU | A | 57 | 56.072 | 51.265 | 86.548 | 1.00 | 51.40 | A | C |
| ATOM | 341 | O | LEU | A | 57 | 55.200 | 50.400 | 86.630 | 1.00 | 54.19 | A | O |
| ATOM | 342 | N | PRO | A | 58 | 56.750 | 51.691 | 87.630 | 1.00 | 50.88 | A | N |
| ATOM | 343 | CD | PRO | A | 58 | 57.998 | 52.478 | 87.636 | 1.00 | 51.30 | A | C |
| ATOM | 344 | CA | PRO | A | 58 | 56.477 | 51.150 | 88.969 | 1.00 | 49.09 | A | C |
| ATOM | 345 | CB | PRO | A | 58 | 57.484 | 51.886 | 89.843 | 1.00 | 49.23 | A | C |
| ATOM | 346 | CG | PRO | A | 58 | 58.649 | 52.031 | 88.927 | 1.00 | 52.26 | A | C |
| ATOM | 347 | C | PRO | A | 58 | 55.034 | 51.417 | 89.402 | 1.00 | 49.77 | A | C |
| ATOM | 348 | O | PRO | A | 58 | 54.391 | 50.573 | 90.042 | 1.00 | 48.08 | A | O |
| ATOM | 349 | N | VAL | A | 59 | 54.537 | 52.604 | 89.060 | 1.00 | 49.94 | A | N |
| ATOM | 350 | CA | VAL | A | 59 | 53.169 | 52.987 | 89.387 | 1.00 | 50.30 | A | C |
| ATOM | 351 | CB | VAL | A | 59 | 52.901 | 54.469 | 89.018 | 1.00 | 51.67 | A | C |
| ATOM | 352 | CG1 | VAL | A | 59 | 51.430 | 54.800 | 89.227 | 1.00 | 51.34 | A | C |
| ATOM | 353 | CG2 | VAL | A | 59 | 53.773 | 55.390 | 89.883 | 1.00 | 51.51 | A | C |
| ATOM | 354 | C | VAL | A | 59 | 52.176 | 52.095 | 88.633 | 1.00 | 49.76 | A | C |
| ATOM | 355 | O | VAL | A | 59 | 51.173 | 51.636 | 89.195 | 1.00 | 48.70 | A | O |
| ATOM | 356 | N | ARG | A | 60 | 52.457 | 51.839 | 87.360 | 1.00 | 47.25 | A | N |
| ATOM | 357 | CA | ARG | A | 60 | 51.561 | 50.998 | 86.563 | 1.00 | 49.97 | A | C |
| ATOM | 358 | CB | ARG | A | 60 | 51.862 | 51.150 | 85.066 | 1.00 | 45.90 | A | C |
| ATOM | 359 | CG | ARG | A | 60 | 51.502 | 52.527 | 84.549 | 1.00 | 45.21 | A | C |
| ATOM | 360 | CD | ARG | A | 60 | 51.516 | 52.603 | 83.041 | 1.00 | 44.24 | A | C |
| ATOM | 361 | NE | ARG | A | 60 | 51.336 | 53.980 | 82.598 | 1.00 | 45.51 | A | N |
| ATOM | 362 | CZ | ARG | A | 60 | 51.167 | 54.347 | 81.334 | 1.00 | 45.62 | A | C |
| ATOM | 363 | NH1 | ARG | A | 60 | 51.147 | 53.438 | 80.371 | 1.00 | 46.43 | A | N |
| ATOM | 364 | NH2 | ARG | A | 60 | 51.029 | 55.627 | 81.033 | 1.00 | 47.73 | A | N |
| ATOM | 365 | C | ARG | A | 60 | 51.663 | 49.540 | 86.979 | 1.00 | 49.54 | A | C |
| ATOM | 366 | O | ARG | A | 60 | 50.652 | 48.852 | 87.110 | 1.00 | 49.28 | A | O |
| ATOM | 367 | N | LEU | A | 61 | 52.887 | 49.069 | 87.198 | 1.00 | 50.62 | A | N |
| ATOM | 368 | CA | LEU | A | 61 | 53.093 | 47.689 | 87.629 | 1.00 | 50.96 | A | C |
| ATOM | 369 | CB | LEU | A | 61 | 54.589 | 47.387 | 87.733 | 1.00 | 53.11 | A | C |
| ATOM | 370 | CG | LEU | A | 61 | 55.371 | 47.345 | 86.420 | 1.00 | 51.62 | A | C |
| ATOM | 371 | CD1 | LEU | A | 61 | 56.857 | 47.302 | 86.717 | 1.00 | 56.31 | A | C |
| ATOM | 372 | CD2 | LEU | A | 61 | 54.954 | 46.132 | 85.616 | 1.00 | 52.75 | A | C |
| ATOM | 373 | C | LEU | A | 61 | 52.423 | 47.443 | 88.981 | 1.00 | 50.11 | A | C |
| ATOM | 374 | O | LEU | A | 61 | 51.762 | 46.420 | 89.177 | 1.00 | 50.09 | A | O |
| ATOM | 375 | N | ALA | A | 62 | 52.570 | 48.398 | 89.898 | 1.00 | 47.96 | A | N |
| ATOM | 376 | CA | ALA | A | 62 | 52.001 | 48.280 | 91.235 | 1.00 | 46.58 | A | C |
| ATOM | 377 | CB | ALA | A | 62 | 52.552 | 49.383 | 92.133 | 1.00 | 48.13 | A | C |
| ATOM | 378 | C | ALA | A | 62 | 50.485 | 48.303 | 91.266 | 1.00 | 47.66 | A | C |
| ATOM | 379 | O | ALA | A | 62 | 49.869 | 47.576 | 92.048 | 1.00 | 49.44 | A | O |
| ATOM | 380 | N | ASN | A | 63 | 49.871 | 49.149 | 90.441 | 1.00 | 48.34 | A | N |
| ATOM | 381 | CA | ASN | A | 63 | 48.412 | 49.218 | 90.415 | 1.00 | 47.94 | A | C |
| ATOM | 382 | CB | ASN | A | 63 | 47.932 | 50.370 | 89.518 | 1.00 | 50.59 | A | C |
| ATOM | 383 | CG | ASN | A | 63 | 47.876 | 51.706 | 90.257 | 1.00 | 48.80 | A | C |
| ATOM | 384 | OD1 | ASN | A | 63 | 48.898 | 52.345 | 90.492 | 1.00 | 51.48 | A | O |
| ATOM | 385 | ND2 | ASN | A | 63 | 46.677 | 52.118 | 90.636 | 1.00 | 48.05 | A | N |
| ATOM | 386 | C | ASN | A | 63 | 47.824 | 47.889 | 89.938 | 1.00 | 47.10 | A | C |
| ATOM | 387 | O | ASN | A | 63 | 46.882 | 47.371 | 90.536 | 1.00 | 46.88 | A | O |
| ATOM | 388 | N | ILE | A | 64 | 48.371 | 47.324 | 88.866 | 1.00 | 46.63 | A | N |
| ATOM | 389 | CA | ILE | A | 64 | 47.845 | 46.044 | 88.408 | 1.00 | 50.16 | A | C |
| ATOM | 390 | CB | ILE | A | 64 | 48.371 | 45.672 | 86.986 | 1.00 | 49.62 | A | C |
| ATOM | 391 | CG2 | ILE | A | 64 | 49.843 | 45.326 | 87.023 | 1.00 | 49.13 | A | C |
| ATOM | 392 | CG1 | ILE | A | 64 | 47.589 | 44.476 | 86.445 | 1.00 | 51.25 | A | C |
| ATOM | 393 | CD1 | ILE | A | 64 | 46.076 | 44.672 | 86.448 | 1.00 | 49.58 | A | C |
| ATOM | 394 | C | ILE | A | 64 | 48.212 | 44.944 | 89.430 | 1.00 | 51.56 | A | C |
| ATOM | 395 | O | ILE | A | 64 | 47.408 | 44.048 | 89.704 | 1.00 | 51.70 | A | O |
| ATOM | 396 | N | MET | A | 65 | 49.408 | 45.020 | 90.013 | 1.00 | 52.30 | A | N |
| ATOM | 397 | CA | MET | A | 65 | 49.809 | 44.019 | 91.006 | 1.00 | 55.16 | A | C |
| ATOM | 398 | CB | MET | A | 65 | 51.266 | 44.205 | 91.410 | 1.00 | 51.46 | A | C |
| ATOM | 399 | CG | MET | A | 65 | 52.240 | 43.757 | 90.359 | 1.00 | 52.66 | A | C |
| ATOM | 400 | SD | MET | A | 65 | 53.894 | 44.296 | 90.757 | 1.00 | 62.73 | A | S |
| ATOM | 401 | CE | MET | A | 65 | 54.360 | 43.001 | 91.965 | 1.00 | 55.60 | A | C |
| ATOM | 402 | C | MET | A | 65 | 48.940 | 44.103 | 92.244 | 1.00 | 57.46 | A | C |
| ATOM | 403 | O | MET | A | 65 | 48.666 | 43.094 | 92.894 | 1.00 | 57.75 | A | O |
| ATOM | 404 | N | LYS | A | 66 | 48.503 | 45.317 | 92.561 | 1.00 | 63.15 | A | N |
| ATOM | 405 | CA | LYS | A | 66 | 47.665 | 45.560 | 93.728 | 1.00 | 67.86 | A | C |
| ATOM | 406 | CB | LYS | A | 66 | 47.572 | 47.071 | 93.983 | 1.00 | 69.75 | A | C |
| ATOM | 407 | CG | LYS | A | 66 | 47.589 | 47.492 | 95.454 | 1.00 | 71.71 | A | C |
| ATOM | 408 | CD | LYS | A | 66 | 46.325 | 47.086 | 96.189 | 1.00 | 73.10 | A | C |
| ATOM | 409 | CE | LYS | A | 66 | 46.403 | 47.472 | 97.659 | 1.00 | 74.14 | A | C |
| ATOM | 410 | NZ | LYS | A | 66 | 45.201 | 47.030 | 98.417 | 1.00 | 76.41 | A | N |
| ATOM | 411 | C | LYS | A | 66 | 46.270 | 44.968 | 93.520 | 1.00 | 70.86 | A | C |
| ATOM | 412 | O | LYS | A | 66 | 45.654 | 44.477 | 94.462 | 1.00 | 71.66 | A | O |
| ATOM | 413 | N | GLU | A | 67 | 45.780 | 45.005 | 92.284 | 1.00 | 74.68 | A | N |
| ATOM | 414 | CA | GLU | A | 67 | 44.452 | 44.481 | 91.977 | 1.00 | 77.68 | A | C |
| ATOM | 415 | CB | GLU | A | 67 | 43.965 | 45.023 | 90.632 | 1.00 | 79.77 | A | C |
| ATOM | 416 | CG | GLU | A | 67 | 42.517 | 44.674 | 90.327 | 1.00 | 83.09 | A | C |
| ATOM | 417 | CD | GLU | A | 67 | 41.939 | 45.493 | 89.189 | 1.00 | 84.71 | A | C |
| ATOM | 418 | OE1 | GLU | A | 67 | 41.877 | 46.737 | 89.312 | 1.00 | 84.24 | A | O |
| ATOM | 419 | OE2 | GLU | A | 67 | 41.542 | 44.890 | 88.173 | 1.00 | 86.19 | A | O |
| ATOM | 420 | C | GLU | A | 67 | 44.423 | 42.954 | 91.964 | 1.00 | 79.00 | A | C |
| ATOM | 421 | O | GLU | A | 67 | 43.428 | 42.330 | 92.349 | 1.00 | 78.07 | A | O |
| ATOM | 422 | N | ILE | A | 68 | 45.515 | 42.352 | 91.511 | 1.00 | 79.08 | A | N |
| ATOM | 423 | CA | ILE | A | 68 | 45.608 | 40.900 | 91.482 | 1.00 | 80.43 | A | C |
| ATOM | 424 | CB | ILE | A | 68 | 46.944 | 40.437 | 90.855 | 1.00 | 77.15 | A | C |
| ATOM | 425 | CG2 | ILE | A | 68 | 47.090 | 38.932 | 90.995 | 1.00 | 75.22 | A | C |
| ATOM | 426 | CG1 | ILE | A | 68 | 47.000 | 40.855 | 89.384 | 1.00 | 71.53 | A | C |
| ATOM | 427 | CD1 | ILE | A | 68 | 48.320 | 40.551 | 88.716 | 1.00 | 68.72 | A | C |
| ATOM | 428 | C | ILE | A | 68 | 45.522 | 40.395 | 92.924 | 1.00 | 84.04 | A | C |
| ATOM | 429 | O | ILE | A | 68 | 44.633 | 39.614 | 93.261 | 1.00 | 84.11 | A | O |
| ATOM | 430 | N | ASN | A | 69 | 46.437 | 40.866 | 93.773 | 1.00 | 87.62 | A | N |
| ATOM | 431 | CA | ASN | A | 69 | 46.473 | 40.471 | 95.179 | 1.00 | 91.41 | A | C |
| ATOM | 432 | CB | ASN | A | 69 | 47.404 | 41.384 | 95.985 | 1.00 | 95.33 | A | C |
| ATOM | 433 | CG | ASN | A | 69 | 48.865 | 41.169 | 95.662 | 1.00 | 99.38 | A | C |
| ATOM | 434 | OD1 | ASN | A | 69 | 49.273 | 40.073 | 95.280 | 1.00 | 101.30 | A | O |
| ATOM | 435 | ND2 | ASN | A | 69 | 49.669 | 42.213 | 95.835 | 1.00 | 102.16 | A | N |
| ATOM | 436 | C | ASN | A | 69 | 45.112 | 40.475 | 95.857 | 1.00 | 92.01 | A | C |
| ATOM | 437 | O | ASN | A | 69 | 44.947 | 39.870 | 96.917 | 1.00 | 92.31 | A | O |
| ATOM | 438 | N | LEU | A | 70 | 44.141 | 41.156 | 95.259 | 1.00 | 93.56 | A | N |
| ATOM | 439 | CA | LEU | A | 70 | 42.815 | 41.229 | 95.854 | 1.00 | 95.98 | A | C |
| ATOM | 440 | CB | LEU | A | 70 | 42.394 | 42.694 | 96.013 | 1.00 | 102.76 | A | C |
| ATOM | 441 | CG | LEU | A | 70 | 43.276 | 43.505 | 96.974 | 1.00 | 108.76 | A | C |
| ATOM | 442 | CD1 | LEU | A | 70 | 42.709 | 44.903 | 97.124 | 1.00 | 111.83 | A | C |
| ATOM | 443 | CD2 | LEU | A | 70 | 43.348 | 42.815 | 98.335 | 1.00 | 112.00 | A | C |
| ATOM | 444 | C | LEU | A | 70 | 41.718 | 40.437 | 95.146 | 1.00 | 94.54 | A | C |
| ATOM | 445 | O | LEU | A | 70 | 40.548 | 40.824 | 95.168 | 1.00 | 94.05 | A | O |
| ATOM | 446 | N | LEU | A | 71 | 42.100 | 39.332 | 94.513 | 1.00 | 92.63 | A | N |
| ATOM | 447 | CA | LEU | A | 71 | 41.138 | 38.456 | 93.856 | 1.00 | 90.61 | A | C |
| ATOM | 448 | CB | LEU | A | 71 | 41.469 | 38.291 | 92.363 | 1.00 | 87.81 | A | C |
| ATOM | 449 | CG | LEU | A | 71 | 42.883 | 38.005 | 91.850 | 1.00 | 85.51 | A | C |
| ATOM | 450 | CD1 | LEU | A | 71 | 43.282 | 36.577 | 92.159 | 1.00 | 86.16 | A | C |
| ATOM | 451 | CD2 | LEU | A | 71 | 42.924 | 38.234 | 90.352 | 1.00 | 82.54 | A | C |
| ATOM | 452 | C | LEU | A | 71 | 41.197 | 37.120 | 94.609 | 1.00 | 90.72 | A | C |
| ATOM | 453 | O | LEU | A | 71 | 42.230 | 36.777 | 95.191 | 1.00 | 90.53 | A | O |
| ATOM | 454 | N | PRO | A | 72 | 40.089 | 36.359 | 94.619 | 1.00 | 90.25 | A | N |
| ATOM | 455 | CD | PRO | A | 72 | 38.925 | 36.526 | 93.728 | 1.00 | 90.05 | A | C |
| ATOM | 456 | CA | PRO | A | 72 | 40.024 | 35.068 | 95.313 | 1.00 | 90.48 | A | C |
| ATOM | 457 | CB | PRO | A | 72 | 38.970 | 34.313 | 94.513 | 1.00 | 90.67 | A | C |
| ATOM | 458 | CG | PRO | A | 72 | 38.014 | 35.396 | 94.161 | 1.00 | 89.59 | A | C |
| ATOM | 459 | C | PRO | A | 72 | 41.353 | 34.312 | 95.402 | 1.00 | 91.54 | A | C |
| ATOM | 460 | O | PRO | A | 72 | 42.102 | 34.233 | 94.427 | 1.00 | 91.56 | A | O |
| ATOM | 461 | N | ASP | A | 73 | 41.645 | 33.767 | 96.581 | 1.00 | 92.42 | A | N |
| ATOM | 462 | CA | ASP | A | 73 | 42.879 | 33.012 | 96.787 | 1.00 | 92.35 | A | C |
| ATOM | 463 | CB | ASP | A | 73 | 43.061 | 32.672 | 98.268 | 1.00 | 97.34 | A | C |
| ATOM | 464 | CG | ASP | A | 73 | 44.347 | 31.912 | 98.538 | 1.00 | 101.68 | A | C |
| ATOM | 465 | OD1 | ASP | A | 73 | 45.434 | 32.434 | 98.202 | 1.00 | 103.05 | A | O |
| ATOM | 466 | OD2 | ASP | A | 73 | 44.270 | 30.790 | 99.087 | 1.00 | 103.68 | A | O |
| ATOM | 467 | C | ASP | A | 73 | 42.824 | 31.732 | 95.964 | 1.00 | 89.66 | A | C |
| ATOM | 468 | O | ASP | A | 73 | 43.826 | 31.040 | 95.789 | 1.00 | 88.79 | A | O |
| ATOM | 469 | N | ARG | A | 74 | 41.633 | 31.426 | 95.464 | 1.00 | 86.88 | A | N |
| ATOM | 470 | CA | ARG | A | 74 | 41.423 | 30.249 | 94.635 | 1.00 | 84.71 | A | C |
| ATOM | 471 | CB | ARG | A | 74 | 39.931 | 30.056 | 94.385 | 1.00 | 85.07 | A | C |
| ATOM | 472 | CG | ARG | A | 74 | 39.059 | 30.356 | 95.588 | 1.00 | 85.46 | A | C |
| ATOM | 473 | CD | ARG | A | 74 | 37.873 | 31.195 | 95.160 | 1.00 | 84.89 | A | C |
| ATOM | 474 | NE | ARG | A | 74 | 37.336 | 30.721 | 93.889 | 1.00 | 81.69 | A | N |
| ATOM | 475 | CZ | ARG | A | 74 | 36.249 | 31.213 | 93.306 | 1.00 | 80.41 | A | C |
| ATOM | 476 | NH1 | ARG | A | 74 | 35.575 | 32.200 | 93.881 | 1.00 | 78.63 | A | N |
| ATOM | 477 | NH2 | ARG | A | 74 | 35.841 | 30.719 | 92.147 | 1.00 | 79.32 | A | N |
| ATOM | 478 | C | ARG | A | 74 | 42.123 | 30.528 | 93.308 | 1.00 | 82.77 | A | C |
| ATOM | 479 | O | ARG | A | 74 | 42.720 | 29.643 | 92.692 | 1.00 | 81.99 | A | O |
| ATOM | 480 | N | VAL | A | 75 | 42.035 | 31.783 | 92.880 | 1.00 | 79.65 | A | N |
| ATOM | 481 | CA | VAL | A | 75 | 42.650 | 32.216 | 91.639 | 1.00 | 75.43 | A | C |
| ATOM | 482 | CB | VAL | A | 75 | 41.956 | 33.480 | 91.104 | 1.00 | 68.82 | A | C |
| ATOM | 483 | CG1 | VAL | A | 75 | 42.414 | 33.766 | 89.688 | 1.00 | 66.17 | A | C |
| ATOM | 484 | CG2 | VAL | A | 75 | 40.444 | 33.295 | 91.150 | 1.00 | 65.24 | A | C |
| ATOM | 485 | C | VAL | A | 75 | 44.120 | 32.507 | 91.907 | 1.00 | 77.99 | A | C |
| ATOM | 486 | O | VAL | A | 75 | 44.990 | 32.140 | 91.124 | 1.00 | 79.04 | A | O |
| ATOM | 487 | N | LEU | A | 76 | 44.392 | 33.147 | 93.035 | 1.00 | 81.91 | A | N |
| ATOM | 488 | CA | LEU | A | 76 | 45.757 | 33.491 | 93.414 | 1.00 | 88.22 | A | C |
| ATOM | 489 | CB | LEU | A | 76 | 45.753 | 34.214 | 94.765 | 1.00 | 87.92 | A | C |
| ATOM | 490 | CG | LEU | A | 76 | 45.188 | 35.632 | 94.812 | 1.00 | 86.73 | A | C |
| ATOM | 491 | CD1 | LEU | A | 76 | 45.057 | 36.096 | 96.256 | 1.00 | 87.06 | A | C |
| ATOM | 492 | CD2 | LEU | A | 76 | 46.109 | 36.548 | 94.033 | 1.00 | 86.87 | A | C |
| ATOM | 493 | C | LEU | A | 76 | 46.706 | 32.295 | 93.499 | 1.00 | 92.26 | A | C |
| ATOM | 494 | O | LEU | A | 76 | 47.811 | 32.327 | 92.953 | 1.00 | 93.13 | A | O |
| ATOM | 495 | N | SER | A | 77 | 46.266 | 31.243 | 94.185 | 1.00 | 96.33 | A | N |
| ATOM | 496 | CA | SER | A | 77 | 47.078 | 30.046 | 94.386 | 1.00 | 99.20 | A | C |
| ATOM | 497 | CB | SER | A | 77 | 46.408 | 29.132 | 95.416 | 1.00 | 101.75 | A | C |
| ATOM | 498 | OG | SER | A | 77 | 46.428 | 29.724 | 96.704 | 1.00 | 105.37 | A | O |
| ATOM | 499 | C | SER | A | 77 | 47.462 | 29.219 | 93.161 | 1.00 | 98.06 | A | C |
| ATOM | 500 | O | SER | A | 77 | 48.334 | 28.354 | 93.258 | 1.00 | 100.58 | A | O |
| ATOM | 501 | N | THR | A | 78 | 46.830 | 29.465 | 92.017 | 1.00 | 94.33 | A | N |
| ATOM | 502 | CA | THR | A | 78 | 47.165 | 28.704 | 90.813 | 1.00 | 89.52 | A | C |
| ATOM | 503 | CB | THR | A | 78 | 46.164 | 28.967 | 89.675 | 1.00 | 88.15 | A | C |
| ATOM | 504 | OG1 | THR | A | 78 | 45.933 | 30.375 | 89.552 | 1.00 | 85.35 | A | O |
| ATOM | 505 | CG2 | THR | A | 78 | 44.854 | 28.254 | 89.947 | 1.00 | 86.42 | A | C |
| ATOM | 506 | C | THR | A | 78 | 48.567 | 29.040 | 90.315 | 1.00 | 87.04 | A | C |
| ATOM | 507 | O | THR | A | 78 | 49.005 | 30.187 | 90.386 | 1.00 | 87.17 | A | O |
| ATOM | 508 | N | PRO | A | 79 | 49.294 | 28.032 | 89.811 | 1.00 | 84.02 | A | N |
| ATOM | 509 | CD | PRO | A | 79 | 48.915 | 26.612 | 89.700 | 1.00 | 82.69 | A | C |
| ATOM | 510 | CA | PRO | A | 79 | 50.652 | 28.248 | 89.307 | 1.00 | 80.57 | A | C |
| ATOM | 511 | CB | PRO | A | 79 | 51.004 | 26.893 | 88.697 | 1.00 | 80.64 | A | C |
| ATOM | 512 | CG | PRO | A | 79 | 50.258 | 25.933 | 89.565 | 1.00 | 82.40 | A | C |
| ATOM | 513 | C | PRO | A | 79 | 50.722 | 29.382 | 88.286 | 1.00 | 77.20 | A | C |
| ATOM | 514 | O | PRO | A | 79 | 51.698 | 30.131 | 88.237 | 1.00 | 76.92 | A | O |
| ATOM | 515 | N | SER | A | 80 | 49.671 | 29.502 | 87.479 | 1.00 | 71.26 | A | N |
| ATOM | 516 | CA | SER | A | 80 | 49.599 | 30.525 | 86.440 | 1.00 | 66.54 | A | C |
| ATOM | 517 | CB | SER | A | 80 | 48.304 | 30.369 | 85.629 | 1.00 | 65.24 | A | C |
| ATOM | 518 | OG | SER | A | 80 | 47.173 | 30.637 | 86.458 | 1.00 | 65.36 | A | O |
| ATOM | 519 | C | SER | A | 80 | 49.650 | 31.933 | 87.020 | 1.00 | 62.00 | A | C |
| ATOM | 520 | O | SER | A | 80 | 50.587 | 32.688 | 86.768 | 1.00 | 59.19 | A | O |
| ATOM | 521 | N | VAL | A | 81 | 48.633 | 32.275 | 87.799 | 1.00 | 59.61 | A | N |
| ATOM | 522 | CA | VAL | A | 81 | 48.562 | 33.586 | 88.414 | 1.00 | 60.67 | A | C |
| ATOM | 523 | CB | VAL | A | 81 | 47.315 | 33.717 | 89.250 | 1.00 | 55.58 | A | C |
| ATOM | 524 | CG1 | VAL | A | 81 | 47.281 | 35.079 | 89.904 | 1.00 | 56.23 | A | C |
| ATOM | 525 | CG2 | VAL | A | 81 | 46.103 | 33.510 | 88.372 | 1.00 | 55.41 | A | C |
| ATOM | 526 | C | VAL | A | 81 | 49.772 | 33.901 | 89.283 | 1.00 | 64.64 | A | C |
| ATOM | 527 | O | VAL | A | 81 | 50.228 | 35.046 | 89.312 | 1.00 | 65.86 | A | O |
| ATOM | 528 | N | GLN | A | 82 | 50.291 | 32.903 | 89.993 | 1.00 | 66.36 | A | N |
| ATOM | 529 | CA | GLN | A | 82 | 51.457 | 33.132 | 90.832 | 1.00 | 70.37 | A | C |
| ATOM | 530 | CB | GLN | A | 82 | 51.610 | 32.026 | 91.882 | 1.00 | 80.81 | A | C |
| ATOM | 531 | CG | GLN | A | 82 | 50.772 | 32.241 | 93.135 | 1.00 | 95.58 | A | C |
| ATOM | 532 | CD | GLN | A | 82 | 51.486 | 31.786 | 94.398 | 1.00 | 104.06 | A | C |
| ATOM | 533 | OE1 | GLN | A | 82 | 52.565 | 32.282 | 94.724 | 1.00 | 108.88 | A | O |
| ATOM | 534 | NE2 | GLN | A | 82 | 50.886 | 30.839 | 95.116 | 1.00 | 108.75 | A | N |
| ATOM | 535 | C | GLN | A | 82 | 52.716 | 33.207 | 89.982 | 1.00 | 67.66 | A | C |
| ATOM | 536 | O | GLN | A | 82 | 53.683 | 33.877 | 90.346 | 1.00 | 66.89 | A | O |
| ATOM | 537 | N | LEU | A | 83 | 52.713 | 32.521 | 88.847 | 1.00 | 63.98 | A | N |
| ATOM | 538 | CA | LEU | A | 83 | 53.878 | 32.550 | 87.979 | 1.00 | 63.36 | A | C |
| ATOM | 539 | CB | LEU | A | 83 | 53.723 | 31.553 | 86.834 | 1.00 | 61.39 | A | C |
| ATOM | 540 | CG | LEU | A | 83 | 54.989 | 31.105 | 86.101 | 1.00 | 61.99 | A | C |
| ATOM | 541 | CD1 | LEU | A | 83 | 54.574 | 30.509 | 84.758 | 1.00 | 61.64 | A | C |
| ATOM | 542 | CD2 | LEU | A | 83 | 55.945 | 32.262 | 85.879 | 1.00 | 61.07 | A | C |
| ATOM | 543 | C | LEU | A | 83 | 54.048 | 33.955 | 87.404 | 1.00 | 65.10 | A | C |
| ATOM | 544 | O | LEU | A | 83 | 55.151 | 34.510 | 87.427 | 1.00 | 64.34 | A | O |
| ATOM | 545 | N | VAL | A | 84 | 52.960 | 34.531 | 86.885 | 1.00 | 65.17 | A | N |
| ATOM | 546 | CA | VAL | A | 84 | 53.038 | 35.872 | 86.308 | 1.00 | 63.92 | A | C |
| ATOM | 547 | CB | VAL | A | 84 | 51.827 | 36.193 | 85.367 | 1.00 | 59.13 | A | C |
| ATOM | 548 | CG1 | VAL | A | 84 | 51.415 | 34.954 | 84.596 | 1.00 | 48.20 | A | C |
| ATOM | 549 | CG2 | VAL | A | 84 | 50.673 | 36.760 | 86.156 | 1.00 | 58.17 | A | C |
| ATOM | 550 | C | VAL | A | 84 | 53.119 | 36.909 | 87.425 | 1.00 | 66.71 | A | C |
| ATOM | 551 | O | VAL | A | 84 | 53.768 | 37.938 | 87.268 | 1.00 | 65.06 | A | O |
| ATOM | 552 | N | GLN | A | 85 | 52.470 | 36.624 | 88.553 | 1.00 | 71.90 | A | N |
| ATOM | 553 | CA | GLN | A | 85 | 52.498 | 37.524 | 89.706 | 1.00 | 77.58 | A | C |
| ATOM | 554 | CB | GLN | A | 85 | 51.605 | 36.989 | 90.833 | 1.00 | 85.38 | A | C |
| ATOM | 555 | CG | GLN | A | 85 | 51.665 | 37.773 | 92.143 | 1.00 | 99.00 | A | C |
| ATOM | 556 | CD | GLN | A | 85 | 51.090 | 39.179 | 92.034 | 1.00 | 108.19 | A | C |
| ATOM | 557 | OE1 | GLN | A | 85 | 51.634 | 40.038 | 91.336 | 1.00 | 113.25 | A | O |
| ATOM | 558 | NE2 | GLN | A | 85 | 49.981 | 39.419 | 92.730 | 1.00 | 112.19 | A | N |
| ATOM | 559 | C | GLN | A | 85 | 53.933 | 37.645 | 90.210 | 1.00 | 76.32 | A | C |
| ATOM | 560 | O | GLN | A | 85 | 54.305 | 38.656 | 90.800 | 1.00 | 77.54 | A | O |
| ATOM | 561 | N | SER | A | 86 | 54.739 | 36.613 | 89.973 | 1.00 | 74.47 | A | N |
| ATOM | 562 | CA | SER | A | 86 | 56.125 | 36.642 | 90.412 | 1.00 | 74.57 | A | C |
| ATOM | 563 | CB | SER | A | 86 | 56.644 | 35.223 | 90.697 | 1.00 | 76.18 | A | C |
| ATOM | 564 | OG | SER | A | 86 | 56.785 | 34.454 | 89.515 | 1.00 | 76.95 | A | O |
| ATOM | 565 | C | SER | A | 86 | 56.974 | 37.315 | 89.343 | 1.00 | 74.12 | A | C |
| ATOM | 566 | O | SER | A | 86 | 58.084 | 37.776 | 89.617 | 1.00 | 74.38 | A | O |
| ATOM | 567 | N | TRP | A | 87 | 56.454 | 37.369 | 88.120 | 1.00 | 74.46 | A | N |
| ATOM | 568 | CA | TRP | A | 87 | 57.171 | 38.024 | 87.030 | 1.00 | 74.35 | A | C |
| ATOM | 569 | CB | TRP | A | 87 | 56.552 | 37.686 | 85.671 | 1.00 | 83.47 | A | C |
| ATOM | 570 | CG | TRP | A | 87 | 56.971 | 36.380 | 85.096 | 1.00 | 93.77 | A | C |
| ATOM | 571 | CD2 | TRP | A | 87 | 56.294 | 35.644 | 84.073 | 1.00 | 99.60 | A | C |
| ATOM | 572 | CE2 | TRP | A | 87 | 57.073 | 34.500 | 83.796 | 1.00 | 101.96 | A | C |
| ATOM | 573 | CE3 | TRP | A | 87 | 55.097 | 35.832 | 83.368 | 1.00 | 102.41 | A | C |
| ATOM | 574 | CD1 | TRP | A | 87 | 58.104 | 35.681 | 85.387 | 1.00 | 97.23 | A | C |
| ATOM | 575 | NE1 | TRP | A | 87 | 58.175 | 34.550 | 84.609 | 1.00 | 100.64 | A | N |
| ATOM | 576 | CZ2 | TRP | A | 87 | 56.703 | 33.555 | 82.832 | 1.00 | 103.72 | A | C |
| ATOM | 577 | CZ3 | TRP | A | 87 | 54.729 | 34.891 | 82.409 | 1.00 | 104.31 | A | C |
| ATOM | 578 | CH2 | TRP | A | 87 | 55.528 | 33.764 | 82.157 | 1.00 | 104.72 | A | C |
| ATOM | 579 | C | TRP | A | 87 | 57.110 | 39.538 | 87.227 | 1.00 | 69.25 | A | C |
| ATOM | 580 | O | TRP | A | 87 | 58.093 | 40.246 | 87.006 | 1.00 | 66.05 | A | O |
| ATOM | 581 | N | TYR | A | 88 | 55.942 | 40.031 | 87.628 | 1.00 | 64.09 | A | N |
| ATOM | 582 | CA | TYR | A | 88 | 55.766 | 41.458 | 87.847 | 1.00 | 62.99 | A | C |
| ATOM | 583 | CB | TYR | A | 88 | 54.280 | 41.795 | 88.009 | 1.00 | 63.98 | A | C |
| ATOM | 584 | CG | TYR | A | 88 | 53.528 | 41.864 | 86.688 | 1.00 | 67.29 | A | C |
| ATOM | 585 | CD1 | TYR | A | 88 | 53.427 | 40.750 | 85.857 | 1.00 | 66.38 | A | C |
| ATOM | 586 | CE1 | TYR | A | 88 | 52.764 | 40.819 | 84.637 | 1.00 | 68.91 | A | C |
| ATOM | 587 | CD2 | TYR | A | 88 | 52.939 | 43.054 | 86.261 | 1.00 | 68.79 | A | C |
| ATOM | 588 | CE2 | TYR | A | 88 | 52.271 | 43.131 | 85.040 | 1.00 | 70.75 | A | C |
| ATOM | 589 | CZ | TYR | A | 88 | 52.189 | 42.012 | 84.236 | 1.00 | 69.56 | A | C |
| ATOM | 590 | OH | TYR | A | 88 | 51.532 | 42.093 | 83.034 | 1.00 | 72.43 | A | O |
| ATOM | 591 | C | TYR | A | 88 | 56.565 | 41.895 | 89.071 | 1.00 | 61.11 | A | C |
| ATOM | 592 | O | TYR | A | 88 | 57.190 | 42.960 | 89.067 | 1.00 | 57.71 | A | O |
| ATOM | 593 | N | VAL | A | 89 | 56.563 | 41.058 | 90.109 | 1.00 | 60.69 | A | N |
| ATOM | 594 | CA | VAL | A | 89 | 57.311 | 41.363 | 91.323 | 1.00 | 57.85 | A | C |
| ATOM | 595 | CB | VAL | A | 89 | 57.197 | 40.221 | 92.352 | 1.00 | 58.40 | A | C |
| ATOM | 596 | CG1 | VAL | A | 89 | 58.184 | 40.448 | 93.489 | 1.00 | 57.87 | A | C |
| ATOM | 597 | CG2 | VAL | A | 89 | 55.763 | 40.141 | 92.895 | 1.00 | 54.35 | A | C |
| ATOM | 598 | C | VAL | A | 89 | 58.779 | 41.572 | 90.956 | 1.00 | 58.59 | A | C |
| ATOM | 599 | O | VAL | A | 89 | 59.412 | 42.518 | 91.421 | 1.00 | 57.28 | A | O |
| ATOM | 600 | N | GLN | A | 90 | 59.311 | 40.701 | 90.104 | 1.00 | 60.00 | A | N |
| ATOM | 601 | CA | GLN | A | 90 | 60.703 | 40.809 | 89.683 | 1.00 | 63.15 | A | C |
| ATOM | 602 | CB | GLN | A | 90 | 61.159 | 39.523 | 88.988 | 1.00 | 67.13 | A | C |
| ATOM | 603 | CG | GLN | A | 90 | 61.696 | 38.459 | 89.942 | 1.00 | 76.91 | A | C |
| ATOM | 604 | CD | GLN | A | 90 | 62.891 | 38.948 | 90.756 | 1.00 | 80.23 | A | C |
| ATOM | 605 | OE1 | GLN | A | 90 | 62.798 | 39.122 | 91.974 | 1.00 | 81.64 | A | O |
| ATOM | 606 | NE2 | GLN | A | 90 | 64.018 | 39.178 | 90.082 | 1.00 | 82.12 | A | N |
| ATOM | 607 | C | GLN | A | 90 | 60.962 | 41.999 | 88.770 | 1.00 | 64.44 | A | C |
| ATOM | 608 | O | GLN | A | 90 | 62.040 | 42.604 | 88.821 | 1.00 | 62.97 | A | O |
| ATOM | 609 | N | SER | A | 91 | 59.990 | 42.330 | 87.921 | 1.00 | 65.21 | A | N |
| ATOM | 610 | CA | SER | A | 91 | 60.145 | 43.471 | 87.023 | 1.00 | 64.06 | A | C |
| ATOM | 611 | CB | SER | A | 91 | 59.042 | 43.489 | 85.957 | 1.00 | 66.14 | A | C |
| ATOM | 612 | OG | SER | A | 91 | 59.302 | 42.541 | 84.941 | 1.00 | 65.66 | A | O |
| ATOM | 613 | C | SER | A | 91 | 60.095 | 44.758 | 87.833 | 1.00 | 60.77 | A | C |
| ATOM | 614 | O | SER | A | 91 | 60.806 | 45.711 | 87.532 | 1.00 | 59.37 | A | O |
| ATOM | 615 | N | LEU | A | 92 | 59.251 | 44.778 | 88.860 | 1.00 | 59.78 | A | N |
| ATOM | 616 | CA | LEU | A | 92 | 59.122 | 45.948 | 89.723 | 1.00 | 62.67 | A | C |
| ATOM | 617 | CB | LEU | A | 92 | 58.013 | 45.715 | 90.757 | 1.00 | 58.61 | A | C |
| ATOM | 618 | CG | LEU | A | 92 | 57.671 | 46.816 | 91.770 | 1.00 | 59.06 | A | C |
| ATOM | 619 | CD1 | LEU | A | 92 | 57.248 | 48.103 | 91.060 | 1.00 | 56.54 | A | C |
| ATOM | 620 | CD2 | LEU | A | 92 | 56.553 | 46.318 | 92.667 | 1.00 | 53.94 | A | C |
| ATOM | 621 | C | LEU | A | 92 | 60.466 | 46.198 | 90.415 | 1.00 | 66.14 | A | C |
| ATOM | 622 | O | LEU | A | 92 | 60.973 | 47.319 | 90.408 | 1.00 | 68.20 | A | O |
| ATOM | 623 | N | LEU | A | 93 | 61.046 | 45.145 | 90.992 | 1.00 | 69.83 | A | N |
| ATOM | 624 | CA | LEU | A | 93 | 62.338 | 45.243 | 91.673 | 1.00 | 71.79 | A | C |
| ATOM | 625 | CB | LEU | A | 93 | 62.679 | 43.913 | 92.348 | 1.00 | 71.95 | A | C |
| ATOM | 626 | CG | LEU | A | 93 | 61.644 | 43.372 | 93.339 | 1.00 | 73.48 | A | C |
| ATOM | 627 | CD1 | LEU | A | 93 | 62.037 | 41.973 | 93.774 | 1.00 | 72.36 | A | C |
| ATOM | 628 | CD2 | LEU | A | 93 | 61.534 | 44.303 | 94.540 | 1.00 | 73.18 | A | C |
| ATOM | 629 | C | LEU | A | 93 | 63.412 | 45.582 | 90.643 | 1.00 | 73.78 | A | C |
| ATOM | 630 | O | LEU | A | 93 | 64.331 | 46.361 | 90.910 | 1.00 | 74.36 | A | O |
| ATOM | 631 | N | ASP | A | 94 | 63.280 | 44.983 | 89.465 | 1.00 | 74.88 | A | N |
| ATOM | 632 | CA | ASP | A | 94 | 64.205 | 45.198 | 88.356 | 1.00 | 76.98 | A | C |
| ATOM | 633 | CB | ASP | A | 94 | 63.703 | 44.457 | 87.113 | 1.00 | 81.41 | A | C |
| ATOM | 634 | CG | ASP | A | 94 | 64.416 | 43.141 | 86.880 | 1.00 | 83.75 | A | C |
| ATOM | 635 | OD1 | ASP | A | 94 | 64.484 | 42.324 | 87.824 | 1.00 | 86.15 | A | O |
| ATOM | 636 | OD2 | ASP | A | 94 | 64.899 | 42.928 | 85.745 | 1.00 | 84.01 | A | O |
| ATOM | 637 | C | ASP | A | 94 | 64.377 | 46.674 | 87.999 | 1.00 | 77.23 | A | C |
| ATOM | 638 | O | ASP | A | 94 | 65.410 | 47.075 | 87.457 | 1.00 | 77.38 | A | O |
| ATOM | 639 | N | ILE | A | 95 | 63.364 | 47.483 | 88.282 | 1.00 | 76.54 | A | N |
| ATOM | 640 | CA | ILE | A | 95 | 63.447 | 48.897 | 87.946 | 1.00 | 76.67 | A | C |
| ATOM | 641 | CB | ILE | A | 95 | 62.177 | 49.367 | 87.200 | 1.00 | 76.87 | A | C |
| ATOM | 642 | CG2 | ILE | A | 95 | 62.425 | 50.726 | 86.551 | 1.00 | 76.88 | A | C |
| ATOM | 643 | CG1 | ILE | A | 95 | 61.823 | 48.359 | 86.104 | 1.00 | 77.04 | A | C |
| ATOM | 644 | CD1 | ILE | A | 95 | 60.484 | 48.608 | 85.443 | 1.00 | 77.14 | A | C |
| ATOM | 645 | C | ILE | A | 95 | 63.679 | 49.787 | 89.162 | 1.00 | 75.78 | A | C |
| ATOM | 646 | O | ILE | A | 95 | 64.009 | 50.963 | 89.018 | 1.00 | 75.37 | A | O |
| ATOM | 647 | N | MET | A | 96 | 63.503 | 49.233 | 90.357 | 1.00 | 75.68 | A | N |
| ATOM | 648 | CA | MET | A | 96 | 63.727 | 50.000 | 91.578 | 1.00 | 77.30 | A | C |
| ATOM | 649 | CB | MET | A | 96 | 63.493 | 49.117 | 92.811 | 1.00 | 76.89 | A | C |
| ATOM | 650 | CG | MET | A | 96 | 62.059 | 48.662 | 92.982 | 1.00 | 74.69 | A | C |
| ATOM | 651 | SD | MET | A | 96 | 60.961 | 50.079 | 93.055 | 1.00 | 75.09 | A | S |
| ATOM | 652 | CE | MET | A | 96 | 59.510 | 49.348 | 93.686 | 1.00 | 73.78 | A | C |
| ATOM | 653 | C | MET | A | 96 | 65.173 | 50.508 | 91.578 | 1.00 | 78.60 | A | C |
| ATOM | 654 | O | MET | A | 96 | 65.446 | 51.677 | 91.869 | 1.00 | 76.81 | A | O |
| ATOM | 655 | N | GLU | A | 97 | 66.086 | 49.608 | 91.232 | 1.00 | 80.99 | A | N |
| ATOM | 656 | CA | GLU | A | 97 | 67.513 | 49.893 | 91.184 | 1.00 | 85.09 | A | C |
| ATOM | 657 | CB | GLU | A | 97 | 68.202 | 48.917 | 90.225 | 1.00 | 89.23 | A | C |
| ATOM | 658 | CG | GLU | A | 97 | 67.847 | 47.451 | 90.447 | 1.00 | 97.98 | A | C |
| ATOM | 659 | CD | GLU | A | 97 | 68.381 | 46.898 | 91.756 | 1.00 | 101.96 | A | C |
| ATOM | 660 | OE1 | GLU | A | 97 | 69.615 | 46.754 | 91.886 | 1.00 | 104.04 | A | O |
| ATOM | 661 | OE2 | GLU | A | 97 | 67.564 | 46.606 | 92.655 | 1.00 | 105.34 | A | O |
| ATOM | 662 | C | GLU | A | 97 | 67.857 | 51.315 | 90.762 | 1.00 | 84.91 | A | C |
| ATOM | 663 | O | GLU | A | 97 | 68.746 | 51.938 | 91.338 | 1.00 | 87.20 | A | O |
| ATOM | 664 | N | PHE | A | 98 | 67.141 | 51.827 | 89.767 | 1.00 | 82.76 | A | N |
| ATOM | 665 | CA | PHE | A | 98 | 67.404 | 53.156 | 89.224 | 1.00 | 80.82 | A | C |
| ATOM | 666 | CB | PHE | A | 98 | 66.956 | 53.193 | 87.757 | 1.00 | 75.45 | A | C |
| ATOM | 667 | CG | PHE | A | 98 | 67.498 | 52.060 | 86.925 | 1.00 | 66.71 | A | C |
| ATOM | 668 | CD1 | PHE | A | 98 | 68.531 | 52.274 | 86.017 | 1.00 | 65.81 | A | C |
| ATOM | 669 | CD2 | PHE | A | 98 | 66.983 | 50.776 | 87.059 | 1.00 | 63.34 | A | C |
| ATOM | 670 | CE1 | PHE | A | 98 | 69.041 | 51.220 | 85.253 | 1.00 | 61.30 | A | C |
| ATOM | 671 | CE2 | PHE | A | 98 | 67.485 | 49.719 | 86.303 | 1.00 | 60.65 | A | C |
| ATOM | 672 | CZ | PHE | A | 98 | 68.514 | 49.941 | 85.400 | 1.00 | 60.29 | A | C |
| ATOM | 673 | C | PHE | A | 98 | 66.794 | 54.349 | 89.965 | 1.00 | 83.63 | A | C |
| ATOM | 674 | O | PHE | A | 98 | 67.026 | 55.492 | 89.577 | 1.00 | 83.82 | A | O |
| ATOM | 675 | N | LEU | A | 99 | 66.029 | 54.100 | 91.023 | 1.00 | 87.77 | A | N |
| ATOM | 676 | CA | LEU | A | 99 | 65.402 | 55.194 | 91.765 | 1.00 | 93.30 | A | C |
| ATOM | 677 | CB | LEU | A | 99 | 64.464 | 54.641 | 92.842 | 1.00 | 92.34 | A | C |
| ATOM | 678 | CG | LEU | A | 99 | 63.110 | 54.129 | 92.340 | 1.00 | 93.38 | A | C |
| ATOM | 679 | CD1 | LEU | A | 99 | 62.320 | 53.552 | 93.505 | 1.00 | 93.35 | A | C |
| ATOM | 680 | CD2 | LEU | A | 99 | 62.340 | 55.269 | 91.678 | 1.00 | 92.81 | A | C |
| ATOM | 681 | C | LEU | A | 99 | 66.373 | 56.192 | 92.396 | 1.00 | 97.57 | A | C |
| ATOM | 682 | O | LEU | A | 99 | 66.070 | 57.384 | 92.479 | 1.00 | 99.15 | A | O |
| ATOM | 683 | N | ASP | A | 100 | 67.532 | 55.714 | 92.841 | 1.00 | 101.79 | A | N |
| ATOM | 684 | CA | ASP | A | 100 | 68.521 | 56.596 | 93.456 | 1.00 | 105.80 | A | C |
| ATOM | 685 | CB | ASP | A | 100 | 69.108 | 55.959 | 94.721 | 1.00 | 109.41 | A | C |
| ATOM | 686 | CG | ASP | A | 100 | 69.637 | 54.557 | 94.483 | 1.00 | 112.46 | A | C |
| ATOM | 687 | OD1 | ASP | A | 100 | 70.119 | 54.277 | 93.367 | 1.00 | 113.43 | A | O |
| ATOM | 688 | OD2 | ASP | A | 100 | 69.586 | 53.736 | 95.423 | 1.00 | 115.56 | A | O |
| ATOM | 689 | C | ASP | A | 100 | 69.650 | 56.947 | 92.493 | 1.00 | 106.14 | A | C |
| ATOM | 690 | O | ASP | A | 100 | 70.512 | 57.769 | 92.808 | 1.00 | 106.85 | A | O |
| ATOM | 691 | N | LYS | A | 101 | 69.643 | 56.319 | 91.321 | 1.00 | 105.84 | A | N |
| ATOM | 692 | CA | LYS | A | 101 | 70.663 | 56.579 | 90.316 | 1.00 | 106.57 | A | C |
| ATOM | 693 | CB | LYS | A | 101 | 70.544 | 55.582 | 89.161 | 1.00 | 103.05 | A | C |
| ATOM | 694 | CG | LYS | A | 101 | 71.316 | 54.287 | 89.362 | 1.00 | 98.77 | A | C |
| ATOM | 695 | CD | LYS | A | 101 | 70.982 | 53.640 | 90.694 | 1.00 | 96.28 | A | C |
| ATOM | 696 | CE | LYS | A | 101 | 71.848 | 52.418 | 90.962 | 1.00 | 95.04 | A | C |
| ATOM | 697 | NZ | LYS | A | 101 | 71.615 | 51.870 | 92.332 | 1.00 | 94.90 | A | N |
| ATOM | 698 | C | LYS | A | 101 | 70.523 | 57.995 | 89.782 | 1.00 | 110.27 | A | C |
| ATOM | 699 | O | LYS | A | 101 | 69.475 | 58.626 | 89.931 | 1.00 | 110.66 | A | O |
| ATOM | 700 | N | ASP | A | 102 | 71.591 | 58.492 | 89.170 | 1.00 | 114.74 | A | N |
| ATOM | 701 | CA | ASP | A | 102 | 71.603 | 59.831 | 88.593 | 1.00 | 119.20 | A | C |
| ATOM | 702 | CB | ASP | A | 102 | 72.698 | 60.699 | 89.238 | 1.00 | 125.41 | A | C |
| ATOM | 703 | CG | ASP | A | 102 | 72.234 | 61.402 | 90.506 | 1.00 | 130.97 | A | C |
| ATOM | 704 | OD1 | ASP | A | 102 | 71.284 | 62.212 | 90.428 | 1.00 | 134.27 | A | O |
| ATOM | 705 | OD2 | ASP | A | 102 | 72.826 | 61.155 | 91.579 | 1.00 | 133.95 | A | O |
| ATOM | 706 | C | ASP | A | 102 | 71.895 | 59.706 | 87.105 | 1.00 | 118.07 | A | C |
| ATOM | 707 | O | ASP | A | 102 | 72.759 | 58.926 | 86.704 | 1.00 | 118.67 | A | O |
| ATOM | 708 | N | PRO | A | 103 | 71.155 | 60.440 | 86.259 | 1.00 | 116.37 | A | N |
| ATOM | 709 | CD | PRO | A | 103 | 69.854 | 61.106 | 86.453 | 1.00 | 115.49 | A | C |
| ATOM | 710 | CA | PRO | A | 103 | 71.467 | 60.316 | 84.835 | 1.00 | 114.67 | A | C |
| ATOM | 711 | CB | PRO | A | 103 | 70.394 | 61.171 | 84.175 | 1.00 | 114.34 | A | C |
| ATOM | 712 | CG | PRO | A | 103 | 69.222 | 60.970 | 85.084 | 1.00 | 114.59 | A | C |
| ATOM | 713 | C | PRO | A | 103 | 72.872 | 60.877 | 84.636 | 1.00 | 114.07 | A | C |
| ATOM | 714 | O | PRO | A | 103 | 73.779 | 60.573 | 85.415 | 1.00 | 114.12 | A | O |
| ATOM | 715 | N | GLU | A | 104 | 73.056 | 61.713 | 83.620 | 1.00 | 112.40 | A | N |
| ATOM | 716 | CA | GLU | A | 104 | 74.372 | 62.287 | 83.361 | 1.00 | 110.43 | A | C |
| ATOM | 717 | CB | GLU | A | 104 | 74.899 | 62.997 | 84.614 | 1.00 | 112.97 | A | C |
| ATOM | 718 | CG | GLU | A | 104 | 74.016 | 64.126 | 85.123 | 1.00 | 116.06 | A | C |
| ATOM | 719 | CD | GLU | A | 104 | 73.950 | 65.290 | 84.157 | 1.00 | 117.89 | A | C |
| ATOM | 720 | OE1 | GLU | A | 104 | 75.020 | 65.839 | 83.815 | 1.00 | 118.91 | A | O |
| ATOM | 721 | OE2 | GLU | A | 104 | 72.831 | 65.660 | 83.744 | 1.00 | 119.33 | A | O |
| ATOM | 722 | C | GLU | A | 104 | 75.333 | 61.167 | 82.967 | 1.00 | 108.40 | A | C |
| ATOM | 723 | O | GLU | A | 104 | 76.084 | 61.294 | 82.002 | 1.00 | 107.02 | A | O |
| ATOM | 724 | N | ASP | A | 105 | 75.299 | 60.073 | 83.724 | 1.00 | 107.23 | A | N |
| ATOM | 725 | CA | ASP | A | 105 | 76.147 | 58.916 | 83.469 | 1.00 | 107.30 | A | C |
| ATOM | 726 | CB | ASP | A | 105 | 76.144 | 57.984 | 84.684 | 1.00 | 109.22 | A | C |
| ATOM | 727 | CG | ASP | A | 105 | 77.125 | 56.834 | 84.543 | 1.00 | 111.28 | A | C |
| ATOM | 728 | OD1 | ASP | A | 105 | 78.341 | 57.094 | 84.451 | 1.00 | 112.89 | A | O |
| ATOM | 729 | OD2 | ASP | A | 105 | 76.679 | 55.668 | 84.527 | 1.00 | 114.36 | A | O |
| ATOM | 730 | C | ASP | A | 105 | 75.611 | 58.177 | 82.250 | 1.00 | 106.80 | A | C |
| ATOM | 731 | O | ASP | A | 105 | 74.624 | 57.445 | 82.338 | 1.00 | 105.05 | A | O |
| ATOM | 732 | N | HIS | A | 106 | 76.268 | 58.380 | 81.112 | 1.00 | 107.91 | A | N |
| ATOM | 733 | CA | HIS | A | 106 | 75.871 | 57.754 | 79.855 | 1.00 | 108.56 | A | C |
| ATOM | 734 | CB | HIS | A | 106 | 76.845 | 58.155 | 78.744 | 1.00 | 112.86 | A | C |
| ATOM | 735 | CG | HIS | A | 106 | 76.542 | 59.486 | 78.129 | 1.00 | 117.58 | A | C |
| ATOM | 736 | CD2 | HIS | A | 106 | 76.774 | 59.967 | 76.883 | 1.00 | 119.95 | A | C |
| ATOM | 737 | ND1 | HIS | A | 106 | 75.935 | 60.509 | 78.825 | 1.00 | 119.22 | A | N |
| ATOM | 738 | CE1 | HIS | A | 106 | 75.804 | 61.561 | 78.036 | 1.00 | 121.02 | A | C |
| ATOM | 739 | NE2 | HIS | A | 106 | 76.306 | 61.257 | 76.852 | 1.00 | 121.44 | A | N |
| ATOM | 740 | C | HIS | A | 106 | 75.768 | 56.237 | 79.930 | 1.00 | 106.88 | A | C |
| ATOM | 741 | O | HIS | A | 106 | 75.428 | 55.587 | 78.943 | 1.00 | 107.38 | A | O |
| ATOM | 742 | N | ARG | A | 107 | 76.063 | 55.671 | 81.097 | 1.00 | 104.63 | A | N |
| ATOM | 743 | CA | ARG | A | 107 | 75.970 | 54.230 | 81.273 | 1.00 | 102.34 | A | C |
| ATOM | 744 | CB | ARG | A | 107 | 77.314 | 53.646 | 81.726 | 1.00 | 106.14 | A | C |
| ATOM | 745 | CG | ARG | A | 107 | 77.224 | 52.164 | 82.024 | 1.00 | 111.71 | A | C |
| ATOM | 746 | CD | ARG | A | 107 | 78.445 | 51.382 | 81.596 | 1.00 | 116.38 | A | C |
| ATOM | 747 | NE | ARG | A | 107 | 78.127 | 49.956 | 81.576 | 1.00 | 121.58 | A | N |
| ATOM | 748 | CZ | ARG | A | 107 | 79.026 | 48.979 | 81.597 | 1.00 | 123.80 | A | C |
| ATOM | 749 | NH1 | ARG | A | 107 | 80.320 | 49.265 | 81.640 | 1.00 | 125.31 | A | N |
| ATOM | 750 | NH2 | ARG | A | 107 | 78.630 | 47.714 | 81.577 | 1.00 | 124.97 | A | N |
| ATOM | 751 | C | ARG | A | 107 | 74.876 | 53.877 | 82.279 | 1.00 | 98.64 | A | C |
| ATOM | 752 | O | ARG | A | 107 | 74.463 | 52.724 | 82.384 | 1.00 | 97.32 | A | O |
| ATOM | 753 | N | THR | A | 108 | 74.408 | 54.877 | 83.016 | 1.00 | 95.54 | A | N |
| ATOM | 754 | CA | THR | A | 108 | 73.349 | 54.672 | 83.998 | 1.00 | 93.92 | A | C |
| ATOM | 755 | CB | THR | A | 108 | 73.243 | 55.872 | 84.969 | 1.00 | 93.67 | A | C |
| ATOM | 756 | OG1 | THR | A | 108 | 74.331 | 55.828 | 85.902 | 1.00 | 94.70 | A | O |
| ATOM | 757 | CG2 | THR | A | 108 | 71.915 | 55.844 | 85.727 | 1.00 | 92.03 | A | C |
| ATOM | 758 | C | THR | A | 108 | 72.012 | 54.504 | 83.285 | 1.00 | 93.05 | A | C |
| ATOM | 759 | O | THR | A | 108 | 71.098 | 53.854 | 83.794 | 1.00 | 92.22 | A | O |
| ATOM | 760 | N | LEU | A | 109 | 71.908 | 55.089 | 82.097 | 1.00 | 91.96 | A | N |
| ATOM | 761 | CA | LEU | A | 109 | 70.677 | 55.014 | 81.330 | 1.00 | 91.57 | A | C |
| ATOM | 762 | CB | LEU | A | 109 | 70.369 | 56.381 | 80.705 | 1.00 | 87.45 | A | C |
| ATOM | 763 | CG | LEU | A | 109 | 71.056 | 56.908 | 79.445 | 1.00 | 84.98 | A | C |
| ATOM | 764 | CD1 | LEU | A | 109 | 70.897 | 58.426 | 79.432 | 1.00 | 81.29 | A | C |
| ATOM | 765 | CD2 | LEU | A | 109 | 72.528 | 56.537 | 79.407 | 1.00 | 85.30 | A | C |
| ATOM | 766 | C | LEU | A | 109 | 70.686 | 53.911 | 80.272 | 1.00 | 93.78 | A | C |
| ATOM | 767 | O | LEU | A | 109 | 69.667 | 53.648 | 79.637 | 1.00 | 95.08 | A | O |
| ATOM | 768 | N | SER | A | 110 | 71.832 | 53.267 | 80.075 | 1.00 | 95.96 | A | N |
| ATOM | 769 | CA | SER | A | 110 | 71.910 | 52.165 | 79.124 | 1.00 | 97.39 | A | C |
| ATOM | 770 | CB | SER | A | 110 | 73.271 | 52.137 | 78.421 | 1.00 | 97.57 | A | C |
| ATOM | 771 | OG | SER | A | 110 | 74.334 | 52.282 | 79.344 | 1.00 | 100.18 | A | O |
| ATOM | 772 | C | SER | A | 110 | 71.693 | 50.905 | 79.956 | 1.00 | 97.98 | A | C |
| ATOM | 773 | O | SER | A | 110 | 71.364 | 49.843 | 79.430 | 1.00 | 98.62 | A | O |
| ATOM | 774 | N | GLN | A | 111 | 71.883 | 51.047 | 81.266 | 1.00 | 99.17 | A | N |
| ATOM | 775 | CA | GLN | A | 111 | 71.671 | 49.959 | 82.217 | 1.00 | 98.88 | A | C |
| ATOM | 776 | CB | GLN | A | 111 | 72.177 | 50.359 | 83.608 | 1.00 | 105.84 | A | C |
| ATOM | 777 | CG | GLN | A | 111 | 73.685 | 50.401 | 83.776 | 1.00 | 114.37 | A | C |
| ATOM | 778 | CD | GLN | A | 111 | 74.277 | 49.034 | 84.044 | 1.00 | 120.32 | A | C |
| ATOM | 779 | OE1 | GLN | A | 111 | 75.493 | 48.888 | 84.174 | 1.00 | 123.84 | A | O |
| ATOM | 780 | NE2 | GLN | A | 111 | 73.418 | 48.022 | 84.133 | 1.00 | 123.58 | A | N |
| ATOM | 781 | C | GLN | A | 111 | 70.157 | 49.803 | 82.270 | 1.00 | 94.37 | A | C |
| ATOM | 782 | O | GLN | A | 111 | 69.627 | 48.749 | 82.620 | 1.00 | 93.82 | A | O |
| ATOM | 783 | N | PHE | A | 112 | 69.478 | 50.888 | 81.912 | 1.00 | 88.06 | A | N |
| ATOM | 784 | CA | PHE | A | 112 | 68.025 | 50.951 | 81.915 | 1.00 | 81.52 | A | C |
| ATOM | 785 | CB | PHE | A | 112 | 67.590 | 52.406 | 82.074 | 1.00 | 74.20 | A | C |
| ATOM | 786 | CG | PHE | A | 112 | 66.156 | 52.572 | 82.461 | 1.00 | 66.88 | A | C |
| ATOM | 787 | CD1 | PHE | A | 112 | 65.688 | 52.065 | 83.665 | 1.00 | 64.47 | A | C |
| ATOM | 788 | CD2 | PHE | A | 112 | 65.275 | 53.253 | 81.632 | 1.00 | 61.95 | A | C |
| ATOM | 789 | CE1 | PHE | A | 112 | 64.363 | 52.236 | 84.037 | 1.00 | 62.91 | A | C |
| ATOM | 790 | CE2 | PHE | A | 112 | 63.954 | 53.429 | 81.994 | 1.00 | 58.08 | A | C |
| ATOM | 791 | CZ | PHE | A | 112 | 63.495 | 52.920 | 83.198 | 1.00 | 59.48 | A | C |
| ATOM | 792 | C | PHE | A | 112 | 67.420 | 50.357 | 80.640 | 1.00 | 80.92 | A | C |
| ATOM | 793 | O | PHE | A | 112 | 66.542 | 49.492 | 80.712 | 1.00 | 80.85 | A | O |
| ATOM | 794 | N | THR | A | 113 | 67.885 | 50.817 | 79.480 | 1.00 | 77.69 | A | N |
| ATOM | 795 | CA | THR | A | 113 | 67.381 | 50.312 | 78.206 | 1.00 | 75.82 | A | C |
| ATOM | 796 | CB | THR | A | 113 | 68.151 | 50.906 | 77.005 | 1.00 | 74.26 | A | C |
| ATOM | 797 | OG1 | THR | A | 113 | 67.990 | 52.330 | 76.990 | 1.00 | 73.08 | A | O |
| ATOM | 798 | CG2 | THR | A | 113 | 67.624 | 50.341 | 75.697 | 1.00 | 71.35 | A | C |
| ATOM | 799 | C | THR | A | 113 | 67.507 | 48.797 | 78.172 | 1.00 | 77.44 | A | C |
| ATOM | 800 | O | THR | A | 113 | 66.657 | 48.109 | 77.618 | 1.00 | 76.76 | A | O |
| ATOM | 801 | N | ASP | A | 114 | 68.574 | 48.278 | 78.769 | 1.00 | 79.88 | A | N |
| ATOM | 802 | CA | ASP | A | 114 | 68.767 | 46.839 | 78.813 | 1.00 | 82.34 | A | C |
| ATOM | 803 | CB | ASP | A | 114 | 70.216 | 46.489 | 79.168 | 1.00 | 86.99 | A | C |
| ATOM | 804 | CG | ASP | A | 114 | 71.154 | 46.601 | 77.974 | 1.00 | 90.48 | A | C |
| ATOM | 805 | OD1 | ASP | A | 114 | 72.312 | 46.142 | 78.075 | 1.00 | 93.43 | A | O |
| ATOM | 806 | OD2 | ASP | A | 114 | 70.731 | 47.148 | 76.933 | 1.00 | 92.23 | A | O |
| ATOM | 807 | C | ASP | A | 114 | 67.820 | 46.252 | 79.845 | 1.00 | 81.45 | A | C |
| ATOM | 808 | O | ASP | A | 114 | 67.272 | 45.172 | 79.646 | 1.00 | 82.06 | A | O |
| ATOM | 809 | N | ALA | A | 115 | 67.627 | 46.974 | 80.946 | 1.00 | 81.21 | A | N |
| ATOM | 810 | CA | ALA | A | 115 | 66.733 | 46.533 | 82.012 | 1.00 | 80.80 | A | C |
| ATOM | 811 | CB | ALA | A | 115 | 66.811 | 47.497 | 83.191 | 1.00 | 81.09 | A | C |
| ATOM | 812 | C | ALA | A | 115 | 65.294 | 46.440 | 81.497 | 1.00 | 80.52 | A | C |
| ATOM | 813 | O | ALA | A | 115 | 64.490 | 45.664 | 82.020 | 1.00 | 81.38 | A | O |
| ATOM | 814 | N | LEU | A | 116 | 64.978 | 47.236 | 80.473 | 1.00 | 77.38 | A | N |
| ATOM | 815 | CA | LEU | A | 116 | 63.640 | 47.237 | 79.871 | 1.00 | 74.89 | A | C |
| ATOM | 816 | CB | LEU | A | 116 | 63.376 | 48.547 | 79.119 | 1.00 | 69.76 | A | C |
| ATOM | 817 | CG | LEU | A | 116 | 63.267 | 49.828 | 79.944 | 1.00 | 67.77 | A | C |
| ATOM | 818 | CD1 | LEU | A | 116 | 62.838 | 50.978 | 79.046 | 1.00 | 67.80 | A | C |
| ATOM | 819 | CD2 | LEU | A | 116 | 62.257 | 49.635 | 81.057 | 1.00 | 65.71 | A | C |
| ATOM | 820 | C | LEU | A | 116 | 63.482 | 46.066 | 78.903 | 1.00 | 74.10 | A | C |
| ATOM | 821 | O | LEU | A | 116 | 62.498 | 45.330 | 78.963 | 1.00 | 72.95 | A | O |
| ATOM | 822 | N | VAL | A | 117 | 64.454 | 45.900 | 78.010 | 1.00 | 72.80 | A | N |
| ATOM | 823 | CA | VAL | A | 117 | 64.423 | 44.808 | 77.042 | 1.00 | 71.76 | A | C |
| ATOM | 824 | CB | VAL | A | 117 | 65.675 | 44.822 | 76.138 | 1.00 | 72.05 | A | C |
| ATOM | 825 | CG1 | VAL | A | 117 | 65.655 | 43.625 | 75.200 | 1.00 | 72.06 | A | C |
| ATOM | 826 | CG2 | VAL | A | 117 | 65.724 | 46.114 | 75.337 | 1.00 | 71.71 | A | C |
| ATOM | 827 | C | VAL | A | 117 | 64.355 | 43.467 | 77.767 | 1.00 | 70.95 | A | C |
| ATOM | 828 | O | VAL | A | 117 | 63.737 | 42.516 | 77.281 | 1.00 | 70.55 | A | O |
| ATOM | 829 | N | THR | A | 118 | 64.989 | 43.402 | 78.934 | 1.00 | 69.50 | A | N |
| ATOM | 830 | CA | THR | A | 118 | 65.008 | 42.186 | 79.741 | 1.00 | 67.98 | A | C |
| ATOM | 831 | CB | THR | A | 118 | 66.125 | 42.249 | 80.810 | 1.00 | 66.33 | A | C |
| ATOM | 832 | OG1 | THR | A | 118 | 67.405 | 42.222 | 80.164 | 1.00 | 67.79 | A | O |
| ATOM | 833 | CG2 | THR | A | 118 | 66.014 | 41.076 | 81.770 | 1.00 | 62.48 | A | C |
| ATOM | 834 | C | THR | A | 118 | 63.661 | 41.936 | 80.429 | 1.00 | 66.94 | A | C |
| ATOM | 835 | O | THR | A | 118 | 63.251 | 40.791 | 80.601 | 1.00 | 67.35 | A | O |
| ATOM | 836 | N | ILE | A | 119 | 62.976 | 43.004 | 80.825 | 1.00 | 64.97 | A | N |
| ATOM | 837 | CA | ILE | A | 119 | 61.675 | 42.869 | 81.474 | 1.00 | 62.13 | A | C |
| ATOM | 838 | CB | ILE | A | 119 | 61.250 | 44.167 | 82.169 | 1.00 | 59.49 | A | C |
| ATOM | 839 | CG2 | ILE | A | 119 | 59.791 | 44.066 | 82.607 | 1.00 | 57.38 | A | C |
| ATOM | 840 | CG1 | ILE | A | 119 | 62.168 | 44.439 | 83.354 | 1.00 | 55.48 | A | C |
| ATOM | 841 | CD1 | ILE | A | 119 | 61.868 | 45.729 | 84.050 | 1.00 | 53.18 | A | C |
| ATOM | 842 | C | ILE | A | 119 | 60.599 | 42.518 | 80.454 | 1.00 | 61.90 | A | C |
| ATOM | 843 | O | ILE | A | 119 | 59.689 | 41.735 | 80.734 | 1.00 | 60.68 | A | O |
| ATOM | 844 | N | ARG | A | 120 | 60.710 | 43.106 | 79.269 | 1.00 | 61.68 | A | N |
| ATOM | 845 | CA | ARG | A | 120 | 59.743 | 42.857 | 78.211 | 1.00 | 63.70 | A | C |
| ATOM | 846 | CB | ARG | A | 120 | 60.109 | 43.664 | 76.968 | 1.00 | 63.45 | A | C |
| ATOM | 847 | CG | ARG | A | 120 | 59.214 | 43.422 | 75.779 | 1.00 | 65.20 | A | C |
| ATOM | 848 | CD | ARG | A | 120 | 59.617 | 44.328 | 74.639 | 1.00 | 68.76 | A | C |
| ATOM | 849 | NE | ARG | A | 120 | 61.029 | 44.173 | 74.301 | 1.00 | 71.60 | A | N |
| ATOM | 850 | CZ | ARG | A | 120 | 61.685 | 44.974 | 73.466 | 1.00 | 75.15 | A | C |
| ATOM | 851 | NH1 | ARG | A | 120 | 61.058 | 45.991 | 72.884 | 1.00 | 73.86 | A | N |
| ATOM | 852 | NH2 | ARG | A | 120 | 62.970 | 44.761 | 73.209 | 1.00 | 76.12 | A | N |
| ATOM | 853 | C | ARG | A | 120 | 59.692 | 41.377 | 77.865 | 1.00 | 64.15 | A | C |
| ATOM | 854 | O | ARG | A | 120 | 58.620 | 40.777 | 77.833 | 1.00 | 63.25 | A | O |
| ATOM | 855 | N | ASN | A | 121 | 60.858 | 40.792 | 77.624 | 1.00 | 65.88 | A | N |
| ATOM | 856 | CA | ASN | A | 121 | 60.940 | 39.387 | 77.265 | 1.00 | 66.99 | A | C |
| ATOM | 857 | CB | ASN | A | 121 | 62.351 | 39.068 | 76.772 | 1.00 | 71.46 | A | C |
| ATOM | 858 | CG | ASN | A | 121 | 62.783 | 39.979 | 75.630 | 1.00 | 75.03 | A | C |
| ATOM | 859 | OD1 | ASN | A | 121 | 61.966 | 40.376 | 74.791 | 1.00 | 75.01 | A | O |
| ATOM | 860 | ND2 | ASN | A | 121 | 64.071 | 40.305 | 75.586 | 1.00 | 77.63 | A | N |
| ATOM | 861 | C | ASN | A | 121 | 60.537 | 38.464 | 78.407 | 1.00 | 65.30 | A | C |
| ATOM | 862 | O | ASN | A | 121 | 60.071 | 37.352 | 78.182 | 1.00 | 65.99 | A | O |
| ATOM | 863 | N | ARG | A | 122 | 60.701 | 38.933 | 79.635 | 1.00 | 64.73 | A | N |
| ATOM | 864 | CA | ARG | A | 122 | 60.331 | 38.149 | 80.813 | 1.00 | 63.28 | A | C |
| ATOM | 865 | CB | ARG | A | 122 | 60.796 | 38.881 | 82.085 | 1.00 | 63.81 | A | C |
| ATOM | 866 | CG | ARG | A | 122 | 60.359 | 38.265 | 83.419 | 1.00 | 63.29 | A | C |
| ATOM | 867 | CD | ARG | A | 122 | 60.865 | 39.111 | 84.598 | 1.00 | 64.58 | A | C |
| ATOM | 868 | NE | ARG | A | 122 | 62.325 | 39.159 | 84.635 | 1.00 | 68.53 | A | N |
| ATOM | 869 | CZ | ARG | A | 122 | 63.037 | 40.229 | 84.980 | 1.00 | 71.36 | A | C |
| ATOM | 870 | NH1 | ARG | A | 122 | 62.426 | 41.356 | 85.325 | 1.00 | 72.27 | A | N |
| ATOM | 871 | NH2 | ARG | A | 122 | 64.365 | 40.180 | 84.961 | 1.00 | 71.14 | A | N |
| ATOM | 872 | C | ARG | A | 122 | 58.814 | 37.922 | 80.857 | 1.00 | 61.42 | A | C |
| ATOM | 873 | O | ARG | A | 122 | 58.341 | 36.933 | 81.417 | 1.00 | 58.85 | A | O |
| ATOM | 874 | N | HIS | A | 123 | 58.062 | 38.841 | 80.250 | 1.00 | 61.55 | A | N |
| ATOM | 875 | CA | HIS | A | 123 | 56.599 | 38.779 | 80.222 | 1.00 | 60.05 | A | C |
| ATOM | 876 | CB | HIS | A | 123 | 56.012 | 40.145 | 80.590 | 1.00 | 57.57 | A | C |
| ATOM | 877 | CG | HIS | A | 123 | 56.366 | 40.615 | 81.964 | 1.00 | 56.01 | A | C |
| ATOM | 878 | CD2 | HIS | A | 123 | 57.537 | 41.046 | 82.485 | 1.00 | 55.91 | A | C |
| ATOM | 879 | ND1 | HIS | A | 123 | 55.443 | 40.690 | 82.985 | 1.00 | 56.75 | A | N |
| ATOM | 880 | CE1 | HIS | A | 123 | 56.032 | 41.148 | 84.076 | 1.00 | 54.64 | A | C |
| ATOM | 881 | NE2 | HIS | A | 123 | 57.302 | 41.372 | 83.799 | 1.00 | 56.93 | A | N |
| ATOM | 882 | C | HIS | A | 123 | 56.014 | 38.368 | 78.865 | 1.00 | 60.77 | A | C |
| ATOM | 883 | O | HIS | A | 123 | 54.796 | 38.366 | 78.700 | 1.00 | 60.70 | A | O |
| ATOM | 884 | N | ASN | A | 124 | 56.860 | 38.012 | 77.905 | 1.00 | 62.52 | A | N |
| ATOM | 885 | CA | ASN | A | 124 | 56.375 | 37.648 | 76.569 | 1.00 | 66.62 | A | C |
| ATOM | 886 | CB | ASN | A | 124 | 57.544 | 37.265 | 75.659 | 1.00 | 68.98 | A | C |
| ATOM | 887 | CG | ASN | A | 124 | 57.923 | 38.377 | 74.710 | 1.00 | 73.50 | A | C |
| ATOM | 888 | OD1 | ASN | A | 124 | 57.056 | 39.005 | 74.100 | 1.00 | 77.04 | A | O |
| ATOM | 889 | ND2 | ASN | A | 124 | 59.219 | 38.625 | 74.570 | 1.00 | 77.26 | A | N |
| ATOM | 890 | C | ASN | A | 124 | 55.304 | 36.572 | 76.445 | 1.00 | 65.91 | A | C |
| ATOM | 891 | O | ASN | A | 124 | 54.545 | 36.562 | 75.476 | 1.00 | 65.99 | A | O |
| ATOM | 892 | N | ASP | A | 125 | 55.226 | 35.674 | 77.414 | 1.00 | 67.06 | A | N |
| ATOM | 893 | CA | ASP | A | 125 | 54.250 | 34.596 | 77.336 | 1.00 | 67.53 | A | C |
| ATOM | 894 | CB | ASP | A | 125 | 54.977 | 33.252 | 77.473 | 1.00 | 72.87 | A | C |
| ATOM | 895 | CG | ASP | A | 125 | 56.110 | 33.091 | 76.456 | 1.00 | 76.79 | A | C |
| ATOM | 896 | OD1 | ASP | A | 125 | 55.826 | 32.910 | 75.251 | 1.00 | 75.81 | A | O |
| ATOM | 897 | OD2 | ASP | A | 125 | 57.291 | 33.160 | 76.864 | 1.00 | 81.86 | A | O |
| ATOM | 898 | C | ASP | A | 125 | 53.141 | 34.705 | 78.380 | 1.00 | 65.57 | A | C |
| ATOM | 899 | O | ASP | A | 125 | 52.554 | 33.695 | 78.771 | 1.00 | 65.24 | A | O |
| ATOM | 900 | N | VAL | A | 126 | 52.842 | 35.927 | 78.815 | 1.00 | 61.71 | A | N |
| ATOM | 901 | CA | VAL | A | 126 | 51.811 | 36.136 | 79.827 | 1.00 | 57.75 | A | C |
| ATOM | 902 | CB | VAL | A | 126 | 51.807 | 37.595 | 80.347 | 1.00 | 56.40 | A | C |
| ATOM | 903 | CG1 | VAL | A | 126 | 50.574 | 37.843 | 81.201 | 1.00 | 54.73 | A | C |
| ATOM | 904 | CG2 | VAL | A | 126 | 53.057 | 37.855 | 81.172 | 1.00 | 56.57 | A | C |
| ATOM | 905 | C | VAL | A | 126 | 50.419 | 35.803 | 79.324 | 1.00 | 57.60 | A | C |
| ATOM | 906 | O | VAL | A | 126 | 49.566 | 35.367 | 80.100 | 1.00 | 56.96 | A | O |
| ATOM | 907 | N | VAL | A | 127 | 50.178 | 36.008 | 78.032 | 1.00 | 57.00 | A | N |
| ATOM | 908 | CA | VAL | A | 127 | 48.861 | 35.718 | 77.490 | 1.00 | 56.89 | A | C |
| ATOM | 909 | CB | VAL | A | 127 | 48.694 | 36.261 | 76.038 | 1.00 | 60.18 | A | C |
| ATOM | 910 | CG1 | VAL | A | 127 | 47.277 | 35.987 | 75.552 | 1.00 | 59.48 | A | C |
| ATOM | 911 | CG2 | VAL | A | 127 | 48.974 | 37.765 | 75.993 | 1.00 | 58.91 | A | C |
| ATOM | 912 | C | VAL | A | 127 | 48.559 | 34.213 | 77.526 | 1.00 | 54.77 | A | C |
| ATOM | 913 | O | VAL | A | 127 | 47.556 | 33.797 | 78.099 | 1.00 | 55.93 | A | O |
| ATOM | 914 | N | PRO | A | 128 | 49.414 | 33.377 | 76.916 | 1.00 | 52.18 | A | N |
| ATOM | 915 | CD | PRO | A | 128 | 50.633 | 33.667 | 76.144 | 1.00 | 51.65 | A | C |
| ATOM | 916 | CA | PRO | A | 128 | 49.144 | 31.934 | 76.944 | 1.00 | 51.85 | A | C |
| ATOM | 917 | CB | PRO | A | 128 | 50.340 | 31.343 | 76.206 | 1.00 | 50.53 | A | C |
| ATOM | 918 | CG | PRO | A | 128 | 50.763 | 32.431 | 75.299 | 1.00 | 51.48 | A | C |
| ATOM | 919 | C | PRO | A | 128 | 49.081 | 31.437 | 78.393 | 1.00 | 53.40 | A | C |
| ATOM | 920 | O | PRO | A | 128 | 48.226 | 30.619 | 78.761 | 1.00 | 53.14 | A | O |
| ATOM | 921 | N | THR | A | 129 | 50.004 | 31.941 | 79.205 | 1.00 | 52.46 | A | N |
| ATOM | 922 | CA | THR | A | 129 | 50.089 | 31.574 | 80.611 | 1.00 | 51.31 | A | C |
| ATOM | 923 | CB | THR | A | 129 | 51.219 | 32.332 | 81.300 | 1.00 | 49.33 | A | C |
| ATOM | 924 | OG1 | THR | A | 129 | 52.472 | 31.943 | 80.723 | 1.00 | 49.67 | A | O |
| ATOM | 925 | CG2 | THR | A | 129 | 51.217 | 32.056 | 82.792 | 1.00 | 44.39 | A | C |
| ATOM | 926 | C | THR | A | 129 | 48.791 | 31.883 | 81.335 | 1.00 | 52.66 | A | C |
| ATOM | 927 | O | THR | A | 129 | 48.271 | 31.049 | 82.073 | 1.00 | 52.56 | A | O |
| ATOM | 928 | N | MET | A | 130 | 48.268 | 33.084 | 81.122 | 1.00 | 54.38 | A | N |
| ATOM | 929 | CA | MET | A | 130 | 47.024 | 33.492 | 81.768 | 1.00 | 55.08 | A | C |
| ATOM | 930 | CB | MET | A | 130 | 46.815 | 34.998 | 81.595 | 1.00 | 57.20 | A | C |
| ATOM | 931 | CG | MET | A | 130 | 47.606 | 35.845 | 82.593 | 1.00 | 61.20 | A | C |
| ATOM | 932 | SD | MET | A | 130 | 46.917 | 35.763 | 84.255 | 1.00 | 60.79 | A | S |
| ATOM | 933 | CE | MET | A | 130 | 47.707 | 37.116 | 85.031 | 1.00 | 62.64 | A | C |
| ATOM | 934 | C | MET | A | 130 | 45.815 | 32.732 | 81.233 | 1.00 | 54.33 | A | C |
| ATOM | 935 | O | MET | A | 130 | 44.847 | 32.507 | 81.956 | 1.00 | 53.83 | A | O |
| ATOM | 936 | N | ALA | A | 131 | 45.867 | 32.340 | 79.967 | 1.00 | 54.22 | A | N |
| ATOM | 937 | CA | ALA | A | 131 | 44.762 | 31.600 | 79.353 | 1.00 | 56.13 | A | C |
| ATOM | 938 | CB | ALA | A | 131 | 45.007 | 31.448 | 77.865 | 1.00 | 49.75 | A | C |
| ATOM | 939 | C | ALA | A | 131 | 44.655 | 30.224 | 80.007 | 1.00 | 58.11 | A | C |
| ATOM | 940 | O | ALA | A | 131 | 43.574 | 29.635 | 80.128 | 1.00 | 57.21 | A | O |
| ATOM | 941 | N | GLN | A | 132 | 45.808 | 29.719 | 80.416 | 1.00 | 60.51 | A | N |
| ATOM | 942 | CA | GLN | A | 132 | 45.901 | 28.435 | 81.073 | 1.00 | 63.13 | A | C |
| ATOM | 943 | CB | GLN | A | 132 | 47.367 | 28.125 | 81.317 | 1.00 | 72.62 | A | C |
| ATOM | 944 | CG | GLN | A | 132 | 47.663 | 26.733 | 81.747 | 1.00 | 88.67 | A | C |
| ATOM | 945 | CD | GLN | A | 132 | 49.112 | 26.417 | 81.522 | 1.00 | 98.33 | A | C |
| ATOM | 946 | OE1 | GLN | A | 132 | 49.551 | 26.260 | 80.382 | 1.00 | 104.97 | A | O |
| ATOM | 947 | NE2 | GLN | A | 132 | 49.877 | 26.348 | 82.600 | 1.00 | 103.29 | A | N |
| ATOM | 948 | C | GLN | A | 132 | 45.138 | 28.514 | 82.394 | 1.00 | 59.80 | A | C |
| ATOM | 949 | O | GLN | A | 132 | 44.453 | 27.567 | 82.780 | 1.00 | 58.36 | A | O |
| ATOM | 950 | N | GLY | A | 133 | 45.254 | 29.653 | 83.079 | 1.00 | 54.35 | A | N |
| ATOM | 951 | CA | GLY | A | 133 | 44.553 | 29.838 | 84.335 | 1.00 | 48.18 | A | C |
| ATOM | 952 | C | GLY | A | 133 | 43.052 | 29.818 | 84.131 | 1.00 | 48.64 | A | C |
| ATOM | 953 | O | GLY | A | 133 | 42.303 | 29.304 | 84.960 | 1.00 | 47.77 | A | O |
| ATOM | 954 | N | VAL | A | 134 | 42.605 | 30.377 | 83.015 | 1.00 | 51.40 | A | N |
| ATOM | 955 | CA | VAL | A | 134 | 41.183 | 30.407 | 82.704 | 1.00 | 50.85 | A | C |
| ATOM | 956 | CB | VAL | A | 134 | 40.923 | 31.134 | 81.379 | 1.00 | 49.34 | A | C |
| ATOM | 957 | CG1 | VAL | A | 134 | 39.445 | 31.041 | 81.009 | 1.00 | 50.17 | A | C |
| ATOM | 958 | CG2 | VAL | A | 134 | 41.356 | 32.587 | 81.502 | 1.00 | 47.15 | A | C |
| ATOM | 959 | C | VAL | A | 134 | 40.661 | 28.985 | 82.589 | 1.00 | 52.23 | A | C |
| ATOM | 960 | O | VAL | A | 134 | 39.605 | 28.656 | 83.128 | 1.00 | 51.86 | A | O |
| ATOM | 961 | N | LEU | A | 135 | 41.406 | 28.145 | 81.877 | 1.00 | 51.81 | A | N |
| ATOM | 962 | CA | LEU | A | 135 | 41.016 | 26.754 | 81.707 | 1.00 | 54.06 | A | C |
| ATOM | 963 | CB | LEU | A | 135 | 41.964 | 26.068 | 80.719 | 1.00 | 54.12 | A | C |
| ATOM | 964 | CG | LEU | A | 135 | 41.533 | 24.701 | 80.184 | 1.00 | 56.06 | A | C |
| ATOM | 965 | CD1 | LEU | A | 135 | 40.146 | 24.784 | 79.565 | 1.00 | 55.68 | A | C |
| ATOM | 966 | CD2 | LEU | A | 135 | 42.548 | 24.225 | 79.164 | 1.00 | 53.51 | A | C |
| ATOM | 967 | C | LEU | A | 135 | 41.044 | 26.046 | 83.074 | 1.00 | 54.50 | A | C |
| ATOM | 968 | O | LEU | A | 135 | 40.120 | 25.313 | 83.425 | 1.00 | 53.89 | A | O |
| ATOM | 969 | N | GLU | A | 136 | 42.102 | 26.275 | 83.844 | 1.00 | 54.93 | A | N |
| ATOM | 970 | CA | GLU | A | 136 | 42.219 | 25.682 | 85.166 | 1.00 | 55.78 | A | C |
| ATOM | 971 | CB | GLU | A | 136 | 43.430 | 26.261 | 85.910 | 1.00 | 60.84 | A | C |
| ATOM | 972 | CG | GLU | A | 136 | 44.654 | 25.353 | 85.953 | 1.00 | 66.05 | A | C |
| ATOM | 973 | CD | GLU | A | 136 | 45.948 | 26.098 | 86.257 | 1.00 | 68.51 | A | C |
| ATOM | 974 | OE1 | GLU | A | 136 | 45.941 | 26.998 | 87.125 | 1.00 | 70.19 | A | O |
| ATOM | 975 | OE2 | GLU | A | 136 | 46.979 | 25.770 | 85.626 | 1.00 | 69.32 | A | O |
| ATOM | 976 | C | GLU | A | 136 | 40.943 | 26.006 | 85.936 | 1.00 | 56.76 | A | C |
| ATOM | 977 | O | GLU | A | 136 | 40.352 | 25.131 | 86.579 | 1.00 | 56.67 | A | O |
| ATOM | 978 | N | TYR | A | 137 | 40.506 | 27.261 | 85.852 | 1.00 | 55.82 | A | N |
| ATOM | 979 | CA | TYR | A | 137 | 39.305 | 27.698 | 86.556 | 1.00 | 53.12 | A | C |
| ATOM | 980 | CB | TYR | A | 137 | 39.171 | 29.228 | 86.470 | 1.00 | 54.12 | A | C |
| ATOM | 981 | CG | TYR | A | 137 | 38.048 | 29.779 | 87.319 | 1.00 | 50.60 | A | C |
| ATOM | 982 | CD1 | TYR | A | 137 | 36.714 | 29.511 | 87.006 | 1.00 | 49.39 | A | C |
| ATOM | 983 | CE1 | TYR | A | 137 | 35.681 | 29.931 | 87.833 | 1.00 | 50.72 | A | C |
| ATOM | 984 | CD2 | TYR | A | 137 | 38.320 | 30.495 | 88.484 | 1.00 | 48.83 | A | C |
| ATOM | 985 | CE2 | TYR | A | 137 | 37.292 | 30.921 | 89.322 | 1.00 | 51.47 | A | C |
| ATOM | 986 | CZ | TYR | A | 137 | 35.976 | 30.632 | 88.990 | 1.00 | 51.82 | A | C |
| ATOM | 987 | OH | TYR | A | 137 | 34.952 | 31.031 | 89.811 | 1.00 | 52.91 | A | O |
| ATOM | 988 | C | TYR | A | 137 | 38.023 | 27.035 | 86.039 | 1.00 | 53.62 | A | C |
| ATOM | 989 | O | TYR | A | 137 | 37.189 | 26.597 | 86.830 | 1.00 | 53.10 | A | O |
| ATOM | 990 | N | LYS | A | 138 | 37.858 | 26.983 | 84.720 | 1.00 | 54.25 | A | N |
| ATOM | 991 | CA | LYS | A | 138 | 36.672 | 26.374 | 84.116 | 1.00 | 55.95 | A | C |
| ATOM | 992 | CB | LYS | A | 138 | 36.794 | 26.349 | 82.582 | 1.00 | 57.09 | A | C |
| ATOM | 993 | CG | LYS | A | 138 | 36.664 | 27.705 | 81.877 | 1.00 | 62.28 | A | C |
| ATOM | 994 | CD | LYS | A | 138 | 37.092 | 27.647 | 80.397 | 1.00 | 61.05 | A | C |
| ATOM | 995 | CE | LYS | A | 138 | 36.259 | 26.667 | 79.574 | 1.00 | 60.20 | A | C |
| ATOM | 996 | NZ | LYS | A | 138 | 34.806 | 26.992 | 79.608 | 1.00 | 62.26 | A | N |
| ATOM | 997 | C | LYS | A | 138 | 36.469 | 24.946 | 84.614 | 1.00 | 57.74 | A | C |
| ATOM | 998 | O | LYS | A | 138 | 35.359 | 24.550 | 84.971 | 1.00 | 58.01 | A | O |
| ATOM | 999 | N | ASP | A | 139 | 37.554 | 24.179 | 84.635 | 1.00 | 57.15 | A | N |
| ATOM | 1000 | CA | ASP | A | 139 | 37.501 | 22.785 | 85.056 | 1.00 | 57.60 | A | C |
| ATOM | 1001 | CB | ASP | A | 139 | 38.671 | 22.013 | 84.432 | 1.00 | 54.59 | A | C |
| ATOM | 1002 | CG | ASP | A | 139 | 38.686 | 22.100 | 82.917 | 1.00 | 54.69 | A | C |
| ATOM | 1003 | OD1 | ASP | A | 139 | 37.601 | 22.008 | 82.307 | 1.00 | 56.76 | A | O |
| ATOM | 1004 | OD2 | ASP | A | 139 | 39.782 | 22.247 | 82.335 | 1.00 | 53.84 | A | O |
| ATOM | 1005 | C | ASP | A | 139 | 37.513 | 22.595 | 86.572 | 1.00 | 58.34 | A | C |
| ATOM | 1006 | O | ASP | A | 139 | 37.023 | 21.589 | 87.089 | 1.00 | 57.98 | A | O |
| ATOM | 1007 | N | THR | A | 140 | 38.066 | 23.566 | 87.285 | 1.00 | 58.07 | A | N |
| ATOM | 1008 | CA | THR | A | 140 | 38.141 | 23.479 | 88.730 | 1.00 | 60.09 | A | C |
| ATOM | 1009 | CB | THR | A | 140 | 39.393 | 24.204 | 89.236 | 1.00 | 61.08 | A | C |
| ATOM | 1010 | OG1 | THR | A | 140 | 40.559 | 23.490 | 88.804 | 1.00 | 66.48 | A | O |
| ATOM | 1011 | CG2 | THR | A | 140 | 39.387 | 24.292 | 90.748 | 1.00 | 64.00 | A | C |
| ATOM | 1012 | C | THR | A | 140 | 36.907 | 24.026 | 89.454 | 1.00 | 61.63 | A | C |
| ATOM | 1013 | O | THR | A | 140 | 36.490 | 23.465 | 90.473 | 1.00 | 59.18 | A | O |
| ATOM | 1014 | N | TYR | A | 141 | 36.322 | 25.108 | 88.930 | 1.00 | 63.81 | A | N |
| ATOM | 1015 | CA | TYR | A | 141 | 35.150 | 25.724 | 89.557 | 1.00 | 65.02 | A | C |
| ATOM | 1016 | CB | TYR | A | 141 | 35.526 | 27.090 | 90.146 | 1.00 | 64.06 | A | C |
| ATOM | 1017 | CG | TYR | A | 141 | 36.492 | 26.980 | 91.302 | 1.00 | 65.48 | A | C |
| ATOM | 1018 | CD1 | TYR | A | 141 | 37.821 | 27.383 | 91.171 | 1.00 | 66.20 | A | C |
| ATOM | 1019 | CE1 | TYR | A | 141 | 38.738 | 27.191 | 92.209 | 1.00 | 69.81 | A | C |
| ATOM | 1020 | CD2 | TYR | A | 141 | 36.097 | 26.390 | 92.505 | 1.00 | 66.48 | A | C |
| ATOM | 1021 | CE2 | TYR | A | 141 | 37.004 | 26.192 | 93.548 | 1.00 | 68.58 | A | C |
| ATOM | 1022 | CZ | TYR | A | 141 | 38.322 | 26.592 | 93.393 | 1.00 | 70.26 | A | C |
| ATOM | 1023 | OH | TYR | A | 141 | 39.227 | 26.371 | 94.410 | 1.00 | 74.74 | A | O |
| ATOM | 1024 | C | TYR | A | 141 | 33.909 | 25.868 | 88.679 | 1.00 | 67.61 | A | C |
| ATOM | 1025 | O | TYR | A | 141 | 32.838 | 26.225 | 89.170 | 1.00 | 66.01 | A | O |
| ATOM | 1026 | N | GLY | A | 142 | 34.039 | 25.580 | 87.388 | 1.00 | 71.50 | A | N |
| ATOM | 1027 | CA | GLY | A | 142 | 32.891 | 25.684 | 86.505 | 1.00 | 76.72 | A | C |
| ATOM | 1028 | C | GLY | A | 142 | 32.828 | 26.992 | 85.744 | 1.00 | 81.47 | A | C |
| ATOM | 1029 | O | GLY | A | 142 | 33.486 | 27.965 | 86.114 | 1.00 | 80.46 | A | O |
| ATOM | 1030 | N | ASP | A | 143 | 32.024 | 27.015 | 84.683 | 1.00 | 87.08 | A | N |
| ATOM | 1031 | CA | ASP | A | 143 | 31.878 | 28.206 | 83.853 | 1.00 | 92.99 | A | C |
| ATOM | 1032 | CB | ASP | A | 143 | 32.241 | 27.874 | 82.399 | 1.00 | 97.44 | A | C |
| ATOM | 1033 | CG | ASP | A | 143 | 31.339 | 26.817 | 81.793 | 1.00 | 102.06 | A | C |
| ATOM | 1034 | OD1 | ASP | A | 143 | 30.713 | 26.049 | 82.554 | 1.00 | 104.54 | A | O |
| ATOM | 1035 | OD2 | ASP | A | 143 | 31.268 | 26.747 | 80.548 | 1.00 | 105.39 | A | O |
| ATOM | 1036 | C | ASP | A | 143 | 30.488 | 28.837 | 83.922 | 1.00 | 94.37 | A | C |
| ATOM | 1037 | O | ASP | A | 143 | 29.657 | 28.654 | 83.034 | 1.00 | 94.66 | A | O |
| ATOM | 1038 | N | ASP | A | 144 | 30.252 | 29.591 | 84.990 | 1.00 | 96.15 | A | N |
| ATOM | 1039 | CA | ASP | A | 144 | 28.984 | 30.275 | 85.202 | 1.00 | 97.56 | A | C |
| ATOM | 1040 | CB | ASP | A | 144 | 28.503 | 30.042 | 86.635 | 1.00 | 95.03 | A | C |
| ATOM | 1041 | CG | ASP | A | 144 | 29.606 | 30.234 | 87.651 | 1.00 | 92.80 | A | C |
| ATOM | 1042 | OD1 | ASP | A | 144 | 30.541 | 29.406 | 87.673 | 1.00 | 91.86 | A | O |
| ATOM | 1043 | OD2 | ASP | A | 144 | 29.545 | 31.215 | 88.421 | 1.00 | 91.02 | A | O |
| ATOM | 1044 | C | ASP | A | 144 | 29.152 | 31.775 | 84.942 | 1.00 | 98.68 | A | C |
| ATOM | 1045 | O | ASP | A | 144 | 30.270 | 32.294 | 84.928 | 1.00 | 100.22 | A | O |
| ATOM | 1046 | N | PRO | A | 145 | 28.039 | 32.491 | 84.729 | 1.00 | 98.12 | A | N |
| ATOM | 1047 | CD | PRO | A | 145 | 26.673 | 31.970 | 84.546 | 1.00 | 98.93 | A | C |
| ATOM | 1048 | CA | PRO | A | 145 | 28.076 | 33.931 | 84.469 | 1.00 | 96.08 | A | C |
| ATOM | 1049 | CB | PRO | A | 145 | 26.599 | 34.303 | 84.462 | 1.00 | 98.47 | A | C |
| ATOM | 1050 | CG | PRO | A | 145 | 25.986 | 33.105 | 83.815 | 1.00 | 98.74 | A | C |
| ATOM | 1051 | C | PRO | A | 145 | 28.904 | 34.794 | 85.430 | 1.00 | 92.96 | A | C |
| ATOM | 1052 | O | PRO | A | 145 | 29.665 | 35.651 | 84.976 | 1.00 | 93.13 | A | O |
| ATOM | 1053 | N | VAL | A | 146 | 28.769 | 34.588 | 86.739 | 1.00 | 87.46 | A | N |
| ATOM | 1054 | CA | VAL | A | 146 | 29.538 | 35.398 | 87.684 | 1.00 | 82.15 | A | C |
| ATOM | 1055 | CB | VAL | A | 146 | 29.118 | 35.137 | 89.147 | 1.00 | 78.22 | A | C |
| ATOM | 1056 | CG1 | VAL | A | 146 | 29.919 | 36.024 | 90.091 | 1.00 | 72.88 | A | C |
| ATOM | 1057 | CG2 | VAL | A | 146 | 27.641 | 35.421 | 89.311 | 1.00 | 74.48 | A | C |
| ATOM | 1058 | C | VAL | A | 146 | 31.041 | 35.154 | 87.534 | 1.00 | 82.19 | A | C |
| ATOM | 1059 | O | VAL | A | 146 | 31.842 | 36.083 | 87.666 | 1.00 | 81.66 | A | O |
| ATOM | 1060 | N | SER | A | 147 | 31.427 | 33.912 | 87.256 | 1.00 | 81.31 | A | N |
| ATOM | 1061 | CA | SER | A | 147 | 32.840 | 33.595 | 87.069 | 1.00 | 81.58 | A | C |
| ATOM | 1062 | CB | SER | A | 147 | 33.041 | 32.086 | 86.905 | 1.00 | 82.89 | A | C |
| ATOM | 1063 | OG | SER | A | 147 | 32.564 | 31.376 | 88.033 | 1.00 | 85.23 | A | O |
| ATOM | 1064 | C | SER | A | 147 | 33.293 | 34.311 | 85.799 | 1.00 | 79.45 | A | C |
| ATOM | 1065 | O | SER | A | 147 | 34.319 | 34.993 | 85.783 | 1.00 | 79.23 | A | O |
| ATOM | 1066 | N | ASN | A | 148 | 32.506 | 34.145 | 84.739 | 1.00 | 77.09 | A | N |
| ATOM | 1067 | CA | ASN | A | 148 | 32.771 | 34.763 | 83.440 | 1.00 | 75.65 | A | C |
| ATOM | 1068 | CB | ASN | A | 148 | 31.610 | 34.477 | 82.491 | 1.00 | 72.84 | A | C |
| ATOM | 1069 | CG | ASN | A | 148 | 31.746 | 35.209 | 81.175 | 1.00 | 72.14 | A | C |
| ATOM | 1070 | OD1 | ASN | A | 148 | 32.559 | 34.840 | 80.329 | 1.00 | 69.60 | A | O |
| ATOM | 1071 | ND2 | ASN | A | 148 | 30.955 | 36.265 | 81.000 | 1.00 | 69.94 | A | N |
| ATOM | 1072 | C | ASN | A | 148 | 32.937 | 36.276 | 83.566 | 1.00 | 74.60 | A | C |
| ATOM | 1073 | O | ASN | A | 148 | 33.855 | 36.868 | 83.001 | 1.00 | 72.54 | A | O |
| ATOM | 1074 | N | GLN | A | 149 | 32.020 | 36.882 | 84.310 | 1.00 | 74.74 | A | N |
| ATOM | 1075 | CA | GLN | A | 149 | 31.993 | 38.315 | 84.556 | 1.00 | 76.98 | A | C |
| ATOM | 1076 | CB | GLN | A | 149 | 30.766 | 38.625 | 85.409 | 1.00 | 85.98 | A | C |
| ATOM | 1077 | CG | GLN | A | 149 | 30.342 | 40.071 | 85.479 | 1.00 | 100.82 | A | C |
| ATOM | 1078 | CD | GLN | A | 149 | 28.996 | 40.218 | 86.171 | 1.00 | 109.20 | A | C |
| ATOM | 1079 | OE1 | GLN | A | 149 | 27.988 | 39.673 | 85.713 | 1.00 | 113.58 | A | O |
| ATOM | 1080 | NE2 | GLN | A | 149 | 28.974 | 40.946 | 87.283 | 1.00 | 114.30 | A | N |
| ATOM | 1081 | C | GLN | A | 149 | 33.268 | 38.794 | 85.257 | 1.00 | 73.18 | A | C |
| ATOM | 1082 | O | GLN | A | 149 | 33.941 | 39.708 | 84.777 | 1.00 | 72.50 | A | O |
| ATOM | 1083 | N | ASN | A | 150 | 33.604 | 38.169 | 86.385 | 1.00 | 69.20 | A | N |
| ATOM | 1084 | CA | ASN | A | 150 | 34.791 | 38.544 | 87.148 | 1.00 | 64.92 | A | C |
| ATOM | 1085 | CB | ASN | A | 150 | 34.860 | 37.743 | 88.450 | 1.00 | 69.59 | A | C |
| ATOM | 1086 | CG | ASN | A | 150 | 33.573 | 37.825 | 89.252 | 1.00 | 73.40 | A | C |
| ATOM | 1087 | OD1 | ASN | A | 150 | 32.950 | 38.884 | 89.341 | 1.00 | 75.68 | A | O |
| ATOM | 1088 | ND2 | ASN | A | 150 | 33.173 | 36.708 | 89.849 | 1.00 | 75.87 | A | N |
| ATOM | 1089 | C | ASN | A | 150 | 36.074 | 38.344 | 86.348 | 1.00 | 60.74 | A | C |
| ATOM | 1090 | O | ASN | A | 150 | 36.983 | 39.169 | 86.413 | 1.00 | 59.43 | A | O |
| ATOM | 1091 | N | ILE | A | 151 | 36.148 | 37.247 | 85.599 | 1.00 | 56.15 | A | N |
| ATOM | 1092 | CA | ILE | A | 151 | 37.319 | 36.961 | 84.771 | 1.00 | 52.51 | A | C |
| ATOM | 1093 | CB | ILE | A | 151 | 37.227 | 35.536 | 84.141 | 1.00 | 50.15 | A | C |
| ATOM | 1094 | CG2 | ILE | A | 151 | 38.281 | 35.364 | 83.038 | 1.00 | 47.12 | A | C |
| ATOM | 1095 | CG1 | ILE | A | 151 | 37.408 | 34.480 | 85.239 | 1.00 | 54.52 | A | C |
| ATOM | 1096 | CD1 | ILE | A | 151 | 37.276 | 33.035 | 84.761 | 1.00 | 55.21 | A | C |
| ATOM | 1097 | C | ILE | A | 151 | 37.401 | 38.008 | 83.654 | 1.00 | 51.17 | A | C |
| ATOM | 1098 | O | ILE | A | 151 | 38.471 | 38.560 | 83.372 | 1.00 | 49.87 | A | O |
| ATOM | 1099 | N | GLN | A | 152 | 36.254 | 38.271 | 83.032 | 1.00 | 49.53 | A | N |
| ATOM | 1100 | CA | GLN | A | 152 | 36.140 | 39.236 | 81.949 | 1.00 | 51.68 | A | C |
| ATOM | 1101 | CB | GLN | A | 152 | 34.682 | 39.322 | 81.491 | 1.00 | 53.90 | A | C |
| ATOM | 1102 | CG | GLN | A | 152 | 34.482 | 39.936 | 80.114 | 1.00 | 57.44 | A | C |
| ATOM | 1103 | CD | GLN | A | 152 | 35.200 | 39.156 | 79.029 | 1.00 | 60.90 | A | C |
| ATOM | 1104 | OE1 | GLN | A | 152 | 35.269 | 37.926 | 79.076 | 1.00 | 60.91 | A | O |
| ATOM | 1105 | NE2 | GLN | A | 152 | 35.727 | 39.867 | 78.038 | 1.00 | 60.64 | A | N |
| ATOM | 1106 | C | GLN | A | 152 | 36.607 | 40.598 | 82.442 | 1.00 | 49.90 | A | C |
| ATOM | 1107 | O | GLN | A | 152 | 37.451 | 41.242 | 81.827 | 1.00 | 49.30 | A | O |
| ATOM | 1108 | N | TYR | A | 153 | 36.055 | 41.020 | 83.570 | 1.00 | 51.04 | A | N |
| ATOM | 1109 | CA | TYR | A | 153 | 36.406 | 42.299 | 84.172 | 1.00 | 53.28 | A | C |
| ATOM | 1110 | CB | TYR | A | 153 | 35.671 | 42.467 | 85.499 | 1.00 | 57.70 | A | C |
| ATOM | 1111 | CG | TYR | A | 153 | 35.936 | 43.797 | 86.154 | 1.00 | 66.41 | A | C |
| ATOM | 1112 | CD1 | TYR | A | 153 | 35.397 | 44.970 | 85.626 | 1.00 | 71.01 | A | C |
| ATOM | 1113 | CE1 | TYR | A | 153 | 35.668 | 46.209 | 86.197 | 1.00 | 73.47 | A | C |
| ATOM | 1114 | CD2 | TYR | A | 153 | 36.756 | 43.895 | 87.279 | 1.00 | 69.31 | A | C |
| ATOM | 1115 | CE2 | TYR | A | 153 | 37.034 | 45.132 | 87.861 | 1.00 | 73.33 | A | C |
| ATOM | 1116 | CZ | TYR | A | 153 | 36.486 | 46.283 | 87.312 | 1.00 | 74.80 | A | C |
| ATOM | 1117 | OH | TYR | A | 153 | 36.761 | 47.507 | 87.871 | 1.00 | 76.91 | A | O |
| ATOM | 1118 | C | TYR | A | 153 | 37.905 | 42.419 | 84.425 | 1.00 | 51.46 | A | C |
| ATOM | 1119 | O | TYR | A | 153 | 38.531 | 43.405 | 84.034 | 1.00 | 49.78 | A | O |
| ATOM | 1120 | N | PHE | A | 154 | 38.467 | 41.406 | 85.085 | 1.00 | 48.02 | A | N |
| ATOM | 1121 | CA | PHE | A | 154 | 39.879 | 41.387 | 85.426 | 1.00 | 42.92 | A | C |
| ATOM | 1122 | CB | PHE | A | 154 | 40.167 | 40.294 | 86.469 | 1.00 | 45.04 | A | C |
| ATOM | 1123 | CG | PHE | A | 154 | 41.637 | 40.016 | 86.657 | 1.00 | 45.78 | A | C |
| ATOM | 1124 | CD1 | PHE | A | 154 | 42.250 | 38.963 | 85.987 | 1.00 | 45.81 | A | C |
| ATOM | 1125 | CD2 | PHE | A | 154 | 42.425 | 40.862 | 87.432 | 1.00 | 43.95 | A | C |
| ATOM | 1126 | CE1 | PHE | A | 154 | 43.631 | 38.759 | 86.079 | 1.00 | 47.37 | A | C |
| ATOM | 1127 | CE2 | PHE | A | 154 | 43.797 | 40.668 | 87.529 | 1.00 | 45.22 | A | C |
| ATOM | 1128 | CZ | PHE | A | 154 | 44.404 | 39.615 | 86.851 | 1.00 | 46.95 | A | C |
| ATOM | 1129 | C | PHE | A | 154 | 40.829 | 41.213 | 84.252 | 1.00 | 41.31 | A | C |
| ATOM | 1130 | O | PHE | A | 154 | 41.837 | 41.912 | 84.166 | 1.00 | 43.01 | A | O |
| ATOM | 1131 | N | LEU | A | 155 | 40.543 | 40.274 | 83.357 | 1.00 | 41.42 | A | N |
| ATOM | 1132 | CA | LEU | A | 155 | 41.443 | 40.070 | 82.227 | 1.00 | 40.20 | A | C |
| ATOM | 1133 | CB | LEU | A | 155 | 41.060 | 38.800 | 81.449 | 1.00 | 43.39 | A | C |
| ATOM | 1134 | CG | LEU | A | 155 | 41.403 | 37.486 | 82.183 | 1.00 | 48.86 | A | C |
| ATOM | 1135 | CD1 | LEU | A | 155 | 41.128 | 36.293 | 81.284 | 1.00 | 46.81 | A | C |
| ATOM | 1136 | CD2 | LEU | A | 155 | 42.875 | 37.488 | 82.585 | 1.00 | 44.49 | A | C |
| ATOM | 1137 | C | LEU | A | 155 | 41.449 | 41.303 | 81.322 | 1.00 | 36.46 | A | C |
| ATOM | 1138 | O | LEU | A | 155 | 42.496 | 41.684 | 80.794 | 1.00 | 36.38 | A | O |
| ATOM | 1139 | N | ASP | A | 156 | 40.298 | 41.943 | 81.154 | 1.00 | 33.55 | A | N |
| ATOM | 1140 | CA | ASP | A | 156 | 40.266 | 43.152 | 80.329 | 1.00 | 39.32 | A | C |
| ATOM | 1141 | CB | ASP | A | 156 | 38.862 | 43.754 | 80.264 | 1.00 | 39.47 | A | C |
| ATOM | 1142 | CG | ASP | A | 156 | 37.981 | 43.084 | 79.222 | 1.00 | 43.30 | A | C |
| ATOM | 1143 | OD1 | ASP | A | 156 | 38.524 | 42.483 | 78.263 | 1.00 | 43.74 | A | O |
| ATOM | 1144 | OD2 | ASP | A | 156 | 36.739 | 43.180 | 79.354 | 1.00 | 47.76 | A | O |
| ATOM | 1145 | C | ASP | A | 156 | 41.219 | 44.188 | 80.909 | 1.00 | 36.91 | A | C |
| ATOM | 1146 | O | ASP | A | 156 | 42.049 | 44.748 | 80.191 | 1.00 | 37.27 | A | O |
| ATOM | 1147 | N | ARG | A | 157 | 41.115 | 44.437 | 82.215 | 1.00 | 37.31 | A | N |
| ATOM | 1148 | CA | ARG | A | 157 | 41.984 | 45.421 | 82.856 | 1.00 | 37.94 | A | C |
| ATOM | 1149 | CB | ARG | A | 157 | 41.513 | 45.682 | 84.284 | 1.00 | 38.96 | A | C |
| ATOM | 1150 | CG | ARG | A | 157 | 40.113 | 46.301 | 84.343 | 1.00 | 43.60 | A | C |
| ATOM | 1151 | CD | ARG | A | 157 | 39.492 | 46.159 | 85.719 | 1.00 | 45.91 | A | C |
| ATOM | 1152 | NE | ARG | A | 157 | 40.195 | 46.962 | 86.712 | 1.00 | 52.27 | A | N |
| ATOM | 1153 | CZ | ARG | A | 157 | 40.112 | 48.287 | 86.793 | 1.00 | 52.30 | A | C |
| ATOM | 1154 | NH1 | ARG | A | 157 | 39.348 | 48.956 | 85.934 | 1.00 | 52.66 | A | N |
| ATOM | 1155 | NH2 | ARG | A | 157 | 40.796 | 48.939 | 87.724 | 1.00 | 50.01 | A | N |
| ATOM | 1156 | C | ARG | A | 157 | 43.446 | 44.994 | 82.844 | 1.00 | 36.97 | A | C |
| ATOM | 1157 | O | ARG | A | 157 | 44.324 | 45.781 | 82.487 | 1.00 | 36.03 | A | O |
| ATOM | 1158 | N | PHE | A | 158 | 43.700 | 43.737 | 83.201 | 1.00 | 40.09 | A | N |
| ATOM | 1159 | CA | PHE | A | 158 | 45.054 | 43.200 | 83.246 | 1.00 | 37.31 | A | C |
| ATOM | 1160 | CB | PHE | A | 158 | 45.029 | 41.712 | 83.624 | 1.00 | 41.69 | A | C |
| ATOM | 1161 | CG | PHE | A | 158 | 46.402 | 41.087 | 83.714 | 1.00 | 43.21 | A | C |
| ATOM | 1162 | CD1 | PHE | A | 158 | 47.266 | 41.406 | 84.766 | 1.00 | 43.92 | A | C |
| ATOM | 1163 | CD2 | PHE | A | 158 | 46.843 | 40.210 | 82.736 | 1.00 | 42.55 | A | C |
| ATOM | 1164 | CE1 | PHE | A | 158 | 48.549 | 40.858 | 84.836 | 1.00 | 45.03 | A | C |
| ATOM | 1165 | CE2 | PHE | A | 158 | 48.129 | 39.655 | 82.796 | 1.00 | 47.16 | A | C |
| ATOM | 1166 | CZ | PHE | A | 158 | 48.982 | 39.981 | 83.849 | 1.00 | 44.31 | A | C |
| ATOM | 1167 | C | PHE | A | 158 | 45.718 | 43.363 | 81.895 | 1.00 | 39.31 | A | C |
| ATOM | 1168 | O | PHE | A | 158 | 46.838 | 43.887 | 81.790 | 1.00 | 39.47 | A | O |
| ATOM | 1169 | N | TYR | A | 159 | 45.030 | 42.910 | 80.853 | 1.00 | 40.09 | A | N |
| ATOM | 1170 | CA | TYR | A | 159 | 45.587 | 43.001 | 79.518 | 1.00 | 42.39 | A | C |
| ATOM | 1171 | CB | TYR | A | 159 | 44.743 | 42.184 | 78.534 | 1.00 | 48.19 | A | C |
| ATOM | 1172 | CG | TYR | A | 159 | 44.923 | 40.683 | 78.681 | 1.00 | 51.17 | A | C |
| ATOM | 1173 | CD1 | TYR | A | 159 | 46.199 | 40.115 | 78.699 | 1.00 | 54.25 | A | C |
| ATOM | 1174 | CE1 | TYR | A | 159 | 46.378 | 38.736 | 78.767 | 1.00 | 56.46 | A | C |
| ATOM | 1175 | CD2 | TYR | A | 159 | 43.825 | 39.827 | 78.741 | 1.00 | 53.23 | A | C |
| ATOM | 1176 | CE2 | TYR | A | 159 | 43.995 | 38.443 | 78.804 | 1.00 | 56.93 | A | C |
| ATOM | 1177 | CZ | TYR | A | 159 | 45.273 | 37.906 | 78.815 | 1.00 | 55.71 | A | C |
| ATOM | 1178 | OH | TYR | A | 159 | 45.446 | 36.543 | 78.848 | 1.00 | 56.14 | A | O |
| ATOM | 1179 | C | TYR | A | 159 | 45.768 | 44.443 | 79.037 | 1.00 | 42.34 | A | C |
| ATOM | 1180 | O | TYR | A | 159 | 46.797 | 44.747 | 78.421 | 1.00 | 40.50 | A | O |
| ATOM | 1181 | N | LEU | A | 160 | 44.809 | 45.337 | 79.311 | 1.00 | 41.01 | A | N |
| ATOM | 1182 | CA | LEU | A | 160 | 44.996 | 46.738 | 78.883 | 1.00 | 42.68 | A | C |
| ATOM | 1183 | CB | LEU | A | 160 | 43.771 | 47.610 | 79.184 | 1.00 | 45.64 | A | C |
| ATOM | 1184 | CG | LEU | A | 160 | 43.064 | 48.246 | 77.974 | 1.00 | 50.96 | A | C |
| ATOM | 1185 | CD1 | LEU | A | 160 | 41.980 | 49.177 | 78.475 | 1.00 | 51.93 | A | C |
| ATOM | 1186 | CD2 | LEU | A | 160 | 44.043 | 49.016 | 77.096 | 1.00 | 47.54 | A | C |
| ATOM | 1187 | C | LEU | A | 160 | 46.201 | 47.279 | 79.648 | 1.00 | 41.32 | A | C |
| ATOM | 1188 | O | LEU | A | 160 | 47.054 | 47.970 | 79.088 | 1.00 | 44.02 | A | O |
| ATOM | 1189 | N | SER | A | 161 | 46.276 | 46.956 | 80.933 | 1.00 | 41.26 | A | N |
| ATOM | 1190 | CA | SER | A | 161 | 47.410 | 47.385 | 81.741 | 1.00 | 42.78 | A | C |
| ATOM | 1191 | CB | SER | A | 161 | 47.337 | 46.760 | 83.141 | 1.00 | 46.07 | A | C |
| ATOM | 1192 | OG | SER | A | 161 | 48.558 | 46.926 | 83.845 | 1.00 | 51.63 | A | O |
| ATOM | 1193 | C | SER | A | 161 | 48.688 | 46.937 | 81.033 | 1.00 | 42.08 | A | C |
| ATOM | 1194 | O | SER | A | 161 | 49.596 | 47.744 | 80.829 | 1.00 | 41.63 | A | O |
| ATOM | 1195 | N | ARG | A | 162 | 48.751 | 45.661 | 80.636 | 1.00 | 39.74 | A | N |
| ATOM | 1196 | CA | ARG | A | 162 | 49.940 | 45.133 | 79.954 | 1.00 | 37.18 | A | C |
| ATOM | 1197 | CB | ARG | A | 162 | 49.770 | 43.638 | 79.652 | 1.00 | 39.67 | A | C |
| ATOM | 1198 | CG | ARG | A | 162 | 50.919 | 43.030 | 78.849 | 1.00 | 41.21 | A | C |
| ATOM | 1199 | CD | ARG | A | 162 | 50.662 | 41.552 | 78.509 | 1.00 | 45.15 | A | C |
| ATOM | 1200 | NE | ARG | A | 162 | 51.885 | 40.849 | 78.101 | 1.00 | 47.46 | A | N |
| ATOM | 1201 | CZ | ARG | A | 162 | 52.197 | 40.531 | 76.852 | 1.00 | 48.96 | A | C |
| ATOM | 1202 | NH1 | ARG | A | 162 | 51.378 | 40.850 | 75.858 | 1.00 | 54.94 | A | N |
| ATOM | 1203 | NH2 | ARG | A | 162 | 53.322 | 39.881 | 76.594 | 1.00 | 49.98 | A | N |
| ATOM | 1204 | C | ARG | A | 162 | 50.238 | 45.879 | 78.652 | 1.00 | 38.85 | A | C |
| ATOM | 1205 | O | ARG | A | 162 | 51.381 | 46.268 | 78.389 | 1.00 | 37.55 | A | O |
| ATOM | 1206 | N | ILE | A | 163 | 49.212 | 46.063 | 77.824 | 1.00 | 38.37 | A | N |
| ATOM | 1207 | CA | ILE | A | 163 | 49.386 | 46.775 | 76.568 | 1.00 | 36.97 | A | C |
| ATOM | 1208 | CB | ILE | A | 163 | 48.020 | 46.958 | 75.851 | 1.00 | 39.25 | A | C |
| ATOM | 1209 | CG2 | ILE | A | 163 | 48.157 | 47.926 | 74.667 | 1.00 | 37.37 | A | C |
| ATOM | 1210 | CG1 | ILE | A | 163 | 47.513 | 45.590 | 75.379 | 1.00 | 36.04 | A | C |
| ATOM | 1211 | CD1 | ILE | A | 163 | 46.105 | 45.599 | 74.899 | 1.00 | 37.07 | A | C |
| ATOM | 1212 | C | ILE | A | 163 | 50.023 | 48.149 | 76.802 | 1.00 | 35.10 | A | C |
| ATOM | 1213 | O | ILE | A | 163 | 50.962 | 48.521 | 76.100 | 1.00 | 36.35 | A | O |
| ATOM | 1214 | N | SER | A | 164 | 49.514 | 48.900 | 77.780 | 1.00 | 34.26 | A | N |
| ATOM | 1215 | CA | SER | A | 164 | 50.043 | 50.241 | 78.071 | 1.00 | 41.42 | A | C |
| ATOM | 1216 | CB | SER | A | 164 | 49.199 | 50.936 | 79.129 | 1.00 | 40.74 | A | C |
| ATOM | 1217 | OG | SER | A | 164 | 49.533 | 50.459 | 80.416 | 1.00 | 47.91 | A | O |
| ATOM | 1218 | C | SER | A | 164 | 51.491 | 50.211 | 78.554 | 1.00 | 42.49 | A | C |
| ATOM | 1219 | O | SER | A | 164 | 52.273 | 51.113 | 78.238 | 1.00 | 45.03 | A | O |
| ATOM | 1220 | N | ILE | A | 165 | 51.850 | 49.185 | 79.324 | 1.00 | 41.69 | A | N |
| ATOM | 1221 | CA | ILE | A | 165 | 53.225 | 49.075 | 79.802 | 1.00 | 42.57 | A | C |
| ATOM | 1222 | CB | ILE | A | 165 | 53.358 | 48.002 | 80.902 | 1.00 | 45.16 | A | C |
| ATOM | 1223 | CG2 | ILE | A | 165 | 54.818 | 47.770 | 81.230 | 1.00 | 44.80 | A | C |
| ATOM | 1224 | CG1 | ILE | A | 165 | 52.614 | 48.461 | 82.160 | 1.00 | 40.07 | A | C |
| ATOM | 1225 | CD1 | ILE | A | 165 | 52.423 | 47.381 | 83.183 | 1.00 | 40.81 | A | C |
| ATOM | 1226 | C | ILE | A | 165 | 54.155 | 48.743 | 78.642 | 1.00 | 42.35 | A | C |
| ATOM | 1227 | O | ILE | A | 165 | 55.234 | 49.328 | 78.516 | 1.00 | 42.34 | A | O |
| ATOM | 1228 | N | ARG | A | 166 | 53.743 | 47.820 | 77.775 | 1.00 | 43.40 | A | N |
| ATOM | 1229 | CA | ARG | A | 166 | 54.589 | 47.473 | 76.641 | 1.00 | 42.98 | A | C |
| ATOM | 1230 | CB | ARG | A | 166 | 54.030 | 46.244 | 75.922 | 1.00 | 48.42 | A | C |
| ATOM | 1231 | CG | ARG | A | 166 | 54.407 | 44.933 | 76.625 | 1.00 | 51.02 | A | C |
| ATOM | 1232 | CD | ARG | A | 166 | 53.579 | 43.753 | 76.158 | 1.00 | 55.53 | A | C |
| ATOM | 1233 | NE | ARG | A | 166 | 53.703 | 43.544 | 74.725 | 1.00 | 64.40 | A | N |
| ATOM | 1234 | CZ | ARG | A | 166 | 54.811 | 43.115 | 74.128 | 1.00 | 70.59 | A | C |
| ATOM | 1235 | NH1 | ARG | A | 166 | 55.891 | 42.842 | 74.850 | 1.00 | 72.08 | A | N |
| ATOM | 1236 | NH2 | ARG | A | 166 | 54.843 | 42.971 | 72.807 | 1.00 | 70.92 | A | N |
| ATOM | 1237 | C | ARG | A | 166 | 54.754 | 48.650 | 75.684 | 1.00 | 42.84 | A | C |
| ATOM | 1238 | O | ARG | A | 166 | 55.809 | 48.810 | 75.078 | 1.00 | 41.55 | A | O |
| ATOM | 1239 | N | MET | A | 167 | 53.722 | 49.483 | 75.568 | 1.00 | 43.51 | A | N |
| ATOM | 1240 | CA | MET | A | 167 | 53.773 | 50.658 | 74.698 | 1.00 | 46.25 | A | C |
| ATOM | 1241 | CB | MET | A | 167 | 52.472 | 51.468 | 74.794 | 1.00 | 49.61 | A | C |
| ATOM | 1242 | CG | MET | A | 167 | 51.756 | 51.744 | 73.465 | 1.00 | 53.09 | A | C |
| ATOM | 1243 | SD | MET | A | 167 | 52.684 | 52.604 | 72.183 | 1.00 | 52.37 | A | S |
| ATOM | 1244 | CE | MET | A | 167 | 51.984 | 54.215 | 72.225 | 1.00 | 55.86 | A | C |
| ATOM | 1245 | C | MET | A | 167 | 54.927 | 51.544 | 75.147 | 1.00 | 45.64 | A | C |
| ATOM | 1246 | O | MET | A | 167 | 55.788 | 51.907 | 74.354 | 1.00 | 47.98 | A | O |
| ATOM | 1247 | N | LEU | A | 168 | 54.934 | 51.904 | 76.425 | 1.00 | 45.36 | A | N |
| ATOM | 1248 | CA | LEU | A | 168 | 55.995 | 52.752 | 76.959 | 1.00 | 47.55 | A | C |
| ATOM | 1249 | CB | LEU | A | 168 | 55.798 | 52.960 | 78.462 | 1.00 | 47.27 | A | C |
| ATOM | 1250 | CG | LEU | A | 168 | 54.468 | 53.602 | 78.853 | 1.00 | 48.54 | A | C |
| ATOM | 1251 | CD1 | LEU | A | 168 | 54.553 | 54.118 | 80.277 | 1.00 | 47.01 | A | C |
| ATOM | 1252 | CD2 | LEU | A | 168 | 54.162 | 54.749 | 77.912 | 1.00 | 47.43 | A | C |
| ATOM | 1253 | C | LEU | A | 168 | 57.367 | 52.140 | 76.699 | 1.00 | 49.18 | A | C |
| ATOM | 1254 | O | LEU | A | 168 | 58.259 | 52.789 | 76.140 | 1.00 | 48.41 | A | O |
| ATOM | 1255 | N | ILE | A | 169 | 57.527 | 50.883 | 77.101 | 1.00 | 48.63 | A | N |
| ATOM | 1256 | CA | ILE | A | 169 | 58.786 | 50.179 | 76.913 | 1.00 | 49.20 | A | C |
| ATOM | 1257 | CB | ILE | A | 169 | 58.682 | 48.734 | 77.391 | 1.00 | 48.58 | A | C |
| ATOM | 1258 | CG2 | ILE | A | 169 | 59.800 | 47.903 | 76.778 | 1.00 | 47.89 | A | C |
| ATOM | 1259 | CG1 | ILE | A | 169 | 58.694 | 48.713 | 78.918 | 1.00 | 50.23 | A | C |
| ATOM | 1260 | CD1 | ILE | A | 169 | 58.509 | 47.354 | 79.517 | 1.00 | 50.84 | A | C |
| ATOM | 1261 | C | ILE | A | 169 | 59.248 | 50.168 | 75.467 | 1.00 | 50.77 | A | C |
| ATOM | 1262 | O | ILE | A | 169 | 60.420 | 50.410 | 75.187 | 1.00 | 50.93 | A | O |
| ATOM | 1263 | N | ASN | A | 170 | 58.336 | 49.878 | 74.548 | 1.00 | 51.86 | A | N |
| ATOM | 1264 | CA | ASN | A | 170 | 58.709 | 49.847 | 73.146 | 1.00 | 54.93 | A | C |
| ATOM | 1265 | CB | ASN | A | 170 | 57.572 | 49.277 | 72.301 | 1.00 | 58.33 | A | C |
| ATOM | 1266 | CG | ASN | A | 170 | 57.277 | 47.826 | 72.629 | 1.00 | 60.70 | A | C |
| ATOM | 1267 | OD1 | ASN | A | 170 | 58.190 | 47.042 | 72.884 | 1.00 | 62.99 | A | O |
| ATOM | 1268 | ND2 | ASN | A | 170 | 56.001 | 47.457 | 72.613 | 1.00 | 61.04 | A | N |
| ATOM | 1269 | C | ASN | A | 170 | 59.090 | 51.236 | 72.648 | 1.00 | 56.11 | A | C |
| ATOM | 1270 | O | ASN | A | 170 | 59.999 | 51.373 | 71.824 | 1.00 | 57.12 | A | O |
| ATOM | 1271 | N | GLN | A | 171 | 58.407 | 52.266 | 73.144 | 1.00 | 54.93 | A | N |
| ATOM | 1272 | CA | GLN | A | 171 | 58.716 | 53.629 | 72.722 | 1.00 | 58.97 | A | C |
| ATOM | 1273 | CB | GLN | A | 171 | 57.707 | 54.622 | 73.303 | 1.00 | 57.93 | A | C |
| ATOM | 1274 | CG | GLN | A | 171 | 56.453 | 54.830 | 72.450 | 1.00 | 57.55 | A | C |
| ATOM | 1275 | CD | GLN | A | 171 | 56.777 | 55.172 | 71.001 | 1.00 | 59.27 | A | C |
| ATOM | 1276 | OE1 | GLN | A | 171 | 56.864 | 54.288 | 70.144 | 1.00 | 58.41 | A | O |
| ATOM | 1277 | NE2 | GLN | A | 171 | 56.970 | 56.458 | 70.725 | 1.00 | 57.34 | A | N |
| ATOM | 1278 | C | GLN | A | 171 | 60.132 | 54.028 | 73.136 | 1.00 | 60.13 | A | C |
| ATOM | 1279 | O | GLN | A | 171 | 60.848 | 54.682 | 72.377 | 1.00 | 60.20 | A | O |
| ATOM | 1280 | N | HIS | A | 172 | 60.530 | 53.626 | 74.339 | 1.00 | 61.67 | A | N |
| ATOM | 1281 | CA | HIS | A | 172 | 61.860 | 53.935 | 74.848 | 1.00 | 63.74 | A | C |
| ATOM | 1282 | CB | HIS | A | 172 | 61.934 | 53.636 | 76.342 | 1.00 | 65.67 | A | C |
| ATOM | 1283 | CG | HIS | A | 172 | 63.157 | 54.189 | 77.005 | 1.00 | 71.21 | A | C |
| ATOM | 1284 | CD2 | HIS | A | 172 | 64.324 | 53.601 | 77.358 | 1.00 | 71.90 | A | C |
| ATOM | 1285 | ND1 | HIS | A | 172 | 63.275 | 55.517 | 77.356 | 1.00 | 72.59 | A | N |
| ATOM | 1286 | CE1 | HIS | A | 172 | 64.461 | 55.722 | 77.899 | 1.00 | 72.39 | A | C |
| ATOM | 1287 | NE2 | HIS | A | 172 | 65.118 | 54.577 | 77.911 | 1.00 | 72.90 | A | N |
| ATOM | 1288 | C | HIS | A | 172 | 62.940 | 53.123 | 74.129 | 1.00 | 64.46 | A | C |
| ATOM | 1289 | O | HIS | A | 172 | 63.862 | 53.682 | 73.534 | 1.00 | 64.88 | A | O |
| ATOM | 1290 | N | THR | A | 173 | 62.824 | 51.802 | 74.190 | 1.00 | 62.57 | A | N |
| ATOM | 1291 | CA | THR | A | 173 | 63.802 | 50.933 | 73.556 | 1.00 | 63.61 | A | C |
| ATOM | 1292 | CB | THR | A | 173 | 63.420 | 49.458 | 73.748 | 1.00 | 64.75 | A | C |
| ATOM | 1293 | OG1 | THR | A | 173 | 62.120 | 49.229 | 73.194 | 1.00 | 66.65 | A | O |
| ATOM | 1294 | CG2 | THR | A | 173 | 63.402 | 49.102 | 75.229 | 1.00 | 60.96 | A | C |
| ATOM | 1295 | C | THR | A | 173 | 63.992 | 51.219 | 72.063 | 1.00 | 64.02 | A | C |
| ATOM | 1296 | O | THR | A | 173 | 65.118 | 51.207 | 71.568 | 1.00 | 65.01 | A | O |
| ATOM | 1297 | N | LEU | A | 174 | 62.902 | 51.481 | 71.346 | 1.00 | 63.87 | A | N |
| ATOM | 1298 | CA | LEU | A | 174 | 62.991 | 51.766 | 69.916 | 1.00 | 62.91 | A | C |
| ATOM | 1299 | CB | LEU | A | 174 | 61.606 | 51.671 | 69.260 | 1.00 | 62.81 | A | C |
| ATOM | 1300 | CG | LEU | A | 174 | 61.103 | 50.316 | 68.744 | 1.00 | 61.21 | A | C |
| ATOM | 1301 | CD1 | LEU | A | 174 | 60.924 | 49.346 | 69.889 | 1.00 | 62.32 | A | C |
| ATOM | 1302 | CD2 | LEU | A | 174 | 59.781 | 50.515 | 68.023 | 1.00 | 61.07 | A | C |
| ATOM | 1303 | C | LEU | A | 174 | 63.604 | 53.132 | 69.605 | 1.00 | 64.31 | A | C |
| ATOM | 1304 | O | LEU | A | 174 | 64.463 | 53.244 | 68.733 | 1.00 | 63.87 | A | O |
| ATOM | 1305 | N | ILE | A | 175 | 63.161 | 54.168 | 70.315 | 1.00 | 66.47 | A | N |
| ATOM | 1306 | CA | ILE | A | 175 | 63.666 | 55.526 | 70.099 | 1.00 | 68.14 | A | C |
| ATOM | 1307 | CB | ILE | A | 175 | 62.832 | 56.570 | 70.876 | 1.00 | 66.49 | A | C |
| ATOM | 1308 | CG2 | ILE | A | 175 | 63.517 | 57.940 | 70.817 | 1.00 | 66.55 | A | C |
| ATOM | 1309 | CG1 | ILE | A | 175 | 61.418 | 56.651 | 70.298 | 1.00 | 63.85 | A | C |
| ATOM | 1310 | CD1 | ILE | A | 175 | 60.533 | 57.664 | 71.000 | 1.00 | 58.66 | A | C |
| ATOM | 1311 | C | ILE | A | 175 | 65.129 | 55.736 | 70.490 | 1.00 | 71.42 | A | C |
| ATOM | 1312 | O | ILE | A | 175 | 65.797 | 56.612 | 69.936 | 1.00 | 72.02 | A | O |
| ATOM | 1313 | N | PHE | A | 176 | 65.629 | 54.945 | 71.436 | 1.00 | 73.16 | A | N |
| ATOM | 1314 | CA | PHE | A | 176 | 67.009 | 55.107 | 71.878 | 1.00 | 77.20 | A | C |
| ATOM | 1315 | CB | PHE | A | 176 | 67.043 | 55.346 | 73.391 | 1.00 | 70.66 | A | C |
| ATOM | 1316 | CG | PHE | A | 176 | 66.259 | 56.552 | 73.820 | 1.00 | 63.87 | A | C |
| ATOM | 1317 | CD1 | PHE | A | 176 | 66.551 | 57.810 | 73.294 | 1.00 | 61.47 | A | C |
| ATOM | 1318 | CD2 | PHE | A | 176 | 65.192 | 56.425 | 74.698 | 1.00 | 61.14 | A | C |
| ATOM | 1319 | CE1 | PHE | A | 176 | 65.784 | 58.924 | 73.630 | 1.00 | 58.99 | A | C |
| ATOM | 1320 | CE2 | PHE | A | 176 | 64.416 | 57.532 | 75.043 | 1.00 | 58.84 | A | C |
| ATOM | 1321 | CZ | PHE | A | 176 | 64.712 | 58.784 | 74.506 | 1.00 | 59.41 | A | C |
| ATOM | 1322 | C | PHE | A | 176 | 67.949 | 53.969 | 71.505 | 1.00 | 82.28 | A | C |
| ATOM | 1323 | O | PHE | A | 176 | 69.087 | 53.932 | 71.965 | 1.00 | 85.12 | A | O |
| ATOM | 1324 | N | ASP | A | 177 | 67.473 | 53.058 | 70.662 | 1.00 | 88.50 | A | N |
| ATOM | 1325 | CA | ASP | A | 177 | 68.259 | 51.914 | 70.196 | 1.00 | 93.67 | A | C |
| ATOM | 1326 | CB | ASP | A | 177 | 68.165 | 51.805 | 68.668 | 1.00 | 97.87 | A | C |
| ATOM | 1327 | CG | ASP | A | 177 | 68.509 | 53.107 | 67.963 | 1.00 | 102.55 | A | C |
| ATOM | 1328 | OD1 | ASP | A | 177 | 69.621 | 53.638 | 68.181 | 1.00 | 105.55 | A | O |
| ATOM | 1329 | OD2 | ASP | A | 177 | 67.664 | 53.599 | 67.183 | 1.00 | 104.02 | A | O |
| ATOM | 1330 | C | ASP | A | 177 | 69.729 | 51.956 | 70.612 | 1.00 | 94.83 | A | C |
| ATOM | 1331 | O | ASP | A | 177 | 70.612 | 52.236 | 69.800 | 1.00 | 95.25 | A | O |
| ATOM | 1332 | N | LYS | A | 186 | 61.113 | 59.454 | 60.571 | 1.00 | 82.46 | A | N |
| ATOM | 1333 | CA | LYS | A | 186 | 59.802 | 59.148 | 60.000 | 1.00 | 82.99 | A | C |
| ATOM | 1334 | CB | LYS | A | 186 | 59.937 | 58.163 | 58.837 | 1.00 | 88.50 | A | C |
| ATOM | 1335 | CG | LYS | A | 186 | 60.946 | 58.573 | 57.779 | 1.00 | 98.45 | A | C |
| ATOM | 1336 | CD | LYS | A | 186 | 62.364 | 58.145 | 58.148 | 1.00 | 104.76 | A | C |
| ATOM | 1337 | CE | LYS | A | 186 | 63.363 | 58.623 | 57.104 | 1.00 | 109.23 | A | C |
| ATOM | 1338 | NZ | LYS | A | 186 | 62.934 | 58.267 | 55.720 | 1.00 | 111.77 | A | N |
| ATOM | 1339 | C | LYS | A | 186 | 58.877 | 58.553 | 61.057 | 1.00 | 77.87 | A | C |
| ATOM | 1340 | O | LYS | A | 186 | 57.754 | 59.006 | 61.240 | 1.00 | 75.91 | A | O |
| ATOM | 1341 | N | HIS | A | 187 | 59.355 | 57.523 | 61.741 | 1.00 | 73.25 | A | N |
| ATOM | 1342 | CA | HIS | A | 187 | 58.576 | 56.882 | 62.784 | 1.00 | 70.02 | A | C |
| ATOM | 1343 | CB | HIS | A | 187 | 58.688 | 55.360 | 62.685 | 1.00 | 69.84 | A | C |
| ATOM | 1344 | CG | HIS | A | 187 | 57.845 | 54.745 | 61.608 | 1.00 | 69.57 | A | C |
| ATOM | 1345 | CD2 | HIS | A | 187 | 56.905 | 53.777 | 61.667 | 1.00 | 68.14 | A | C |
| ATOM | 1346 | ND1 | HIS | A | 187 | 57.971 | 55.078 | 60.274 | 1.00 | 69.33 | A | N |
| ATOM | 1347 | CE1 | HIS | A | 187 | 57.144 | 54.334 | 59.560 | 1.00 | 67.44 | A | C |
| ATOM | 1348 | NE2 | HIS | A | 187 | 56.486 | 53.535 | 60.379 | 1.00 | 68.53 | A | N |
| ATOM | 1349 | C | HIS | A | 187 | 59.115 | 57.317 | 64.136 | 1.00 | 68.57 | A | C |
| ATOM | 1350 | O | HIS | A | 187 | 60.326 | 57.427 | 64.318 | 1.00 | 69.38 | A | O |
| ATOM | 1351 | N | ILE | A | 188 | 58.219 | 57.576 | 65.080 | 1.00 | 65.29 | A | N |
| ATOM | 1352 | CA | ILE | A | 188 | 58.631 | 57.955 | 66.421 | 1.00 | 62.55 | A | C |
| ATOM | 1353 | CB | ILE | A | 188 | 57.846 | 59.176 | 66.936 | 1.00 | 62.54 | A | C |
| ATOM | 1354 | CG2 | ILE | A | 188 | 58.139 | 59.402 | 68.408 | 1.00 | 58.31 | A | C |
| ATOM | 1355 | CG1 | ILE | A | 188 | 58.228 | 60.410 | 66.115 | 1.00 | 64.86 | A | C |
| ATOM | 1356 | CD1 | ILE | A | 188 | 57.510 | 61.681 | 66.531 | 1.00 | 68.21 | A | C |
| ATOM | 1357 | C | ILE | A | 188 | 58.354 | 56.747 | 67.302 | 1.00 | 61.49 | A | C |
| ATOM | 1358 | O | ILE | A | 188 | 57.283 | 56.624 | 67.897 | 1.00 | 61.71 | A | O |
| ATOM | 1359 | N | GLY | A | 189 | 59.331 | 55.849 | 67.365 | 1.00 | 59.64 | A | N |
| ATOM | 1360 | CA | GLY | A | 189 | 59.181 | 54.644 | 68.154 | 1.00 | 57.34 | A | C |
| ATOM | 1361 | C | GLY | A | 189 | 58.330 | 53.661 | 67.371 | 1.00 | 56.49 | A | C |
| ATOM | 1362 | O | GLY | A | 189 | 58.727 | 53.202 | 66.299 | 1.00 | 55.99 | A | O |
| ATOM | 1363 | N | SER | A | 190 | 57.155 | 53.334 | 67.897 | 1.00 | 53.21 | A | N |
| ATOM | 1364 | CA | SER | A | 190 | 56.261 | 52.412 | 67.214 | 1.00 | 52.36 | A | C |
| ATOM | 1365 | CB | SER | A | 190 | 55.710 | 51.387 | 68.192 | 1.00 | 50.40 | A | C |
| ATOM | 1366 | OG | SER | A | 190 | 54.743 | 51.998 | 69.028 | 1.00 | 56.62 | A | O |
| ATOM | 1367 | C | SER | A | 190 | 55.097 | 53.205 | 66.626 | 1.00 | 51.99 | A | C |
| ATOM | 1368 | O | SER | A | 190 | 54.088 | 52.630 | 66.232 | 1.00 | 51.16 | A | O |
| ATOM | 1369 | N | ILE | A | 191 | 55.256 | 54.524 | 66.567 | 1.00 | 50.31 | A | N |
| ATOM | 1370 | CA | ILE | A | 191 | 54.225 | 55.414 | 66.056 | 1.00 | 50.66 | A | C |
| ATOM | 1371 | CB | ILE | A | 191 | 54.025 | 56.613 | 67.008 | 1.00 | 50.09 | A | C |
| ATOM | 1372 | CG2 | ILE | A | 191 | 53.035 | 57.602 | 66.415 | 1.00 | 46.85 | A | C |
| ATOM | 1373 | CG1 | ILE | A | 191 | 53.536 | 56.104 | 68.364 | 1.00 | 47.87 | A | C |
| ATOM | 1374 | CD1 | ILE | A | 191 | 53.335 | 57.174 | 69.365 | 1.00 | 46.65 | A | C |
| ATOM | 1375 | C | ILE | A | 191 | 54.509 | 55.956 | 64.657 | 1.00 | 51.75 | A | C |
| ATOM | 1376 | O | ILE | A | 191 | 55.596 | 56.469 | 64.383 | 1.00 | 53.31 | A | O |
| ATOM | 1377 | N | ASP | A | 192 | 53.524 | 55.832 | 63.772 | 1.00 | 48.86 | A | N |
| ATOM | 1378 | CA | ASP | A | 192 | 53.654 | 56.337 | 62.416 | 1.00 | 48.33 | A | C |
| ATOM | 1379 | CB | ASP | A | 192 | 53.125 | 55.336 | 61.399 | 1.00 | 47.88 | A | C |
| ATOM | 1380 | CG | ASP | A | 192 | 53.500 | 55.704 | 59.986 | 1.00 | 46.52 | A | C |
| ATOM | 1381 | OD1 | ASP | A | 192 | 53.611 | 56.914 | 59.696 | 1.00 | 48.02 | A | O |
| ATOM | 1382 | OD2 | ASP | A | 192 | 53.679 | 54.788 | 59.162 | 1.00 | 49.64 | A | O |
| ATOM | 1383 | C | ASP | A | 192 | 52.792 | 57.579 | 62.371 | 1.00 | 49.71 | A | C |
| ATOM | 1384 | O | ASP | A | 192 | 51.580 | 57.502 | 62.552 | 1.00 | 49.23 | A | O |
| ATOM | 1385 | N | PRO | A | 193 | 53.404 | 58.746 | 62.144 | 1.00 | 52.72 | A | N |
| ATOM | 1386 | CD | PRO | A | 193 | 54.849 | 59.025 | 62.058 | 1.00 | 53.06 | A | C |
| ATOM | 1387 | CA | PRO | A | 193 | 52.624 | 59.984 | 62.092 | 1.00 | 53.84 | A | C |
| ATOM | 1388 | CB | PRO | A | 193 | 53.679 | 61.056 | 62.330 | 1.00 | 53.18 | A | C |
| ATOM | 1389 | CG | PRO | A | 193 | 54.878 | 60.475 | 61.636 | 1.00 | 53.91 | A | C |
| ATOM | 1390 | C | PRO | A | 193 | 51.873 | 60.155 | 60.771 | 1.00 | 53.52 | A | C |
| ATOM | 1391 | O | PRO | A | 193 | 51.033 | 61.043 | 60.646 | 1.00 | 51.77 | A | O |
| ATOM | 1392 | N | ASN | A | 194 | 52.178 | 59.293 | 59.800 | 1.00 | 54.47 | A | N |
| ATOM | 1393 | CA | ASN | A | 194 | 51.531 | 59.314 | 58.485 | 1.00 | 57.98 | A | C |
| ATOM | 1394 | CB | ASN | A | 194 | 52.483 | 59.858 | 57.421 | 1.00 | 67.41 | A | C |
| ATOM | 1395 | CG | ASN | A | 194 | 52.562 | 61.367 | 57.429 | 1.00 | 77.02 | A | C |
| ATOM | 1396 | OD1 | ASN | A | 194 | 52.871 | 61.979 | 58.450 | 1.00 | 83.25 | A | O |
| ATOM | 1397 | ND2 | ASN | A | 194 | 52.286 | 61.978 | 56.284 | 1.00 | 84.27 | A | N |
| ATOM | 1398 | C | ASN | A | 194 | 51.089 | 57.912 | 58.077 | 1.00 | 55.21 | A | C |
| ATOM | 1399 | O | ASN | A | 194 | 51.279 | 57.501 | 56.931 | 1.00 | 52.83 | A | O |
| ATOM | 1400 | N | CYS | A | 195 | 50.499 | 57.187 | 59.023 | 1.00 | 51.31 | A | N |
| ATOM | 1401 | CA | CYS | A | 195 | 50.035 | 55.826 | 58.789 | 1.00 | 48.60 | A | C |
| ATOM | 1402 | CB | CYS | A | 195 | 49.490 | 55.242 | 60.099 | 1.00 | 46.18 | A | C |
| ATOM | 1403 | SG | CYS | A | 195 | 48.932 | 53.522 | 60.000 | 1.00 | 44.54 | A | S |
| ATOM | 1404 | C | CYS | A | 195 | 48.956 | 55.766 | 57.708 | 1.00 | 46.55 | A | C |
| ATOM | 1405 | O | CYS | A | 195 | 47.895 | 56.370 | 57.860 | 1.00 | 46.24 | A | O |
| ATOM | 1406 | N | ASN | A | 196 | 49.240 | 55.059 | 56.612 | 1.00 | 45.87 | A | N |
| ATOM | 1407 | CA | ASN | A | 196 | 48.279 | 54.901 | 55.517 | 1.00 | 45.59 | A | C |
| ATOM | 1408 | CB | ASN | A | 196 | 48.989 | 54.737 | 54.164 | 1.00 | 46.22 | A | C |
| ATOM | 1409 | CG | ASN | A | 196 | 48.007 | 54.581 | 53.004 | 1.00 | 50.10 | A | C |
| ATOM | 1410 | OD1 | ASN | A | 196 | 47.322 | 53.567 | 52.887 | 1.00 | 53.98 | A | O |
| ATOM | 1411 | ND2 | ASN | A | 196 | 47.928 | 55.593 | 52.151 | 1.00 | 53.47 | A | N |
| ATOM | 1412 | C | ASN | A | 196 | 47.460 | 53.653 | 55.831 | 1.00 | 44.74 | A | C |
| ATOM | 1413 | O | ASN | A | 196 | 47.881 | 52.522 | 55.559 | 1.00 | 45.79 | A | O |
| ATOM | 1414 | N | VAL | A | 197 | 46.285 | 53.869 | 56.409 | 1.00 | 42.63 | A | N |
| ATOM | 1415 | CA | VAL | A | 197 | 45.418 | 52.773 | 56.809 | 1.00 | 40.65 | A | C |
| ATOM | 1416 | CB | VAL | A | 197 | 44.062 | 53.316 | 57.296 | 1.00 | 35.09 | A | C |
| ATOM | 1417 | CG1 | VAL | A | 197 | 43.149 | 52.169 | 57.701 | 1.00 | 35.56 | A | C |
| ATOM | 1418 | CG2 | VAL | A | 197 | 44.290 | 54.229 | 58.486 | 1.00 | 35.47 | A | C |
| ATOM | 1419 | C | VAL | A | 197 | 45.193 | 51.695 | 55.752 | 1.00 | 40.69 | A | C |
| ATOM | 1420 | O | VAL | A | 197 | 45.269 | 50.507 | 56.063 | 1.00 | 40.97 | A | O |
| ATOM | 1421 | N | SER | A | 198 | 44.925 | 52.101 | 54.514 | 1.00 | 40.47 | A | N |
| ATOM | 1422 | CA | SER | A | 198 | 44.681 | 51.144 | 53.439 | 1.00 | 45.20 | A | C |
| ATOM | 1423 | CB | SER | A | 198 | 44.294 | 51.864 | 52.144 | 1.00 | 47.67 | A | C |
| ATOM | 1424 | OG | SER | A | 198 | 42.889 | 52.016 | 52.052 | 1.00 | 52.45 | A | O |
| ATOM | 1425 | C | SER | A | 198 | 45.865 | 50.246 | 53.159 | 1.00 | 46.16 | A | C |
| ATOM | 1426 | O | SER | A | 198 | 45.686 | 49.126 | 52.685 | 1.00 | 48.20 | A | O |
| ATOM | 1427 | N | GLU | A | 199 | 47.073 | 50.731 | 53.435 | 1.00 | 47.84 | A | N |
| ATOM | 1428 | CA | GLU | A | 199 | 48.268 | 49.924 | 53.189 | 1.00 | 48.90 | A | C |
| ATOM | 1429 | CB | GLU | A | 199 | 49.528 | 50.802 | 53.199 | 1.00 | 53.49 | A | C |
| ATOM | 1430 | CG | GLU | A | 199 | 50.841 | 50.060 | 52.912 | 1.00 | 62.37 | A | C |
| ATOM | 1431 | CD | GLU | A | 199 | 50.780 | 49.174 | 51.669 | 1.00 | 66.87 | A | C |
| ATOM | 1432 | OE1 | GLU | A | 199 | 50.203 | 49.604 | 50.644 | 1.00 | 70.19 | A | O |
| ATOM | 1433 | OE2 | GLU | A | 199 | 51.320 | 48.046 | 51.717 | 1.00 | 68.95 | A | O |
| ATOM | 1434 | C | GLU | A | 199 | 48.353 | 48.859 | 54.271 | 1.00 | 44.87 | A | C |
| ATOM | 1435 | O | GLU | A | 199 | 48.699 | 47.702 | 54.001 | 1.00 | 41.81 | A | O |
| ATOM | 1436 | N | VAL | A | 200 | 48.031 | 49.251 | 55.501 | 1.00 | 41.77 | A | N |
| ATOM | 1437 | CA | VAL | A | 200 | 48.063 | 48.306 | 56.610 | 1.00 | 40.68 | A | C |
| ATOM | 1438 | CB | VAL | A | 200 | 47.844 | 48.983 | 57.960 | 1.00 | 41.44 | A | C |
| ATOM | 1439 | CG1 | VAL | A | 200 | 47.894 | 47.937 | 59.059 | 1.00 | 43.48 | A | C |
| ATOM | 1440 | CG2 | VAL | A | 200 | 48.904 | 50.034 | 58.195 | 1.00 | 40.08 | A | C |
| ATOM | 1441 | C | VAL | A | 200 | 46.959 | 47.278 | 56.417 | 1.00 | 42.59 | A | C |
| ATOM | 1442 | O | VAL | A | 200 | 47.081 | 46.137 | 56.868 | 1.00 | 44.06 | A | O |
| ATOM | 1443 | N | VAL | A | 201 | 45.874 | 47.684 | 55.756 | 1.00 | 41.56 | A | N |
| ATOM | 1444 | CA | VAL | A | 201 | 44.775 | 46.766 | 55.493 | 1.00 | 39.33 | A | C |
| ATOM | 1445 | CB | VAL | A | 201 | 43.552 | 47.491 | 54.849 | 1.00 | 39.87 | A | C |
| ATOM | 1446 | CG1 | VAL | A | 201 | 42.610 | 46.472 | 54.222 | 1.00 | 33.70 | A | C |
| ATOM | 1447 | CG2 | VAL | A | 201 | 42.806 | 48.309 | 55.909 | 1.00 | 33.85 | A | C |
| ATOM | 1448 | C | VAL | A | 201 | 45.290 | 45.696 | 54.543 | 1.00 | 39.19 | A | C |
| ATOM | 1449 | O | VAL | A | 201 | 45.143 | 44.505 | 54.810 | 1.00 | 36.64 | A | O |
| ATOM | 1450 | N | LYS | A | 202 | 45.901 | 46.129 | 53.442 | 1.00 | 41.10 | A | N |
| ATOM | 1451 | CA | LYS | A | 202 | 46.440 | 45.200 | 52.446 | 1.00 | 46.06 | A | C |
| ATOM | 1452 | CB | LYS | A | 202 | 47.115 | 45.970 | 51.303 | 1.00 | 48.01 | A | C |
| ATOM | 1453 | CG | LYS | A | 202 | 46.168 | 46.790 | 50.460 | 1.00 | 54.96 | A | C |
| ATOM | 1454 | CD | LYS | A | 202 | 46.911 | 47.624 | 49.429 | 1.00 | 62.00 | A | C |
| ATOM | 1455 | CE | LYS | A | 202 | 45.975 | 48.636 | 48.773 | 1.00 | 67.83 | A | C |
| ATOM | 1456 | NZ | LYS | A | 202 | 46.703 | 49.792 | 48.175 | 1.00 | 71.41 | A | N |
| ATOM | 1457 | C | LYS | A | 202 | 47.441 | 44.209 | 53.043 | 1.00 | 46.18 | A | C |
| ATOM | 1458 | O | LYS | A | 202 | 47.429 | 43.020 | 52.695 | 1.00 | 47.12 | A | O |
| ATOM | 1459 | N | ASP | A | 203 | 48.300 | 44.694 | 53.939 | 1.00 | 45.56 | A | N |
| ATOM | 1460 | CA | ASP | A | 203 | 49.311 | 43.839 | 54.565 | 1.00 | 46.87 | A | C |
| ATOM | 1461 | CB | ASP | A | 203 | 50.268 | 44.657 | 55.439 | 1.00 | 47.53 | A | C |
| ATOM | 1462 | CG | ASP | A | 203 | 51.092 | 45.652 | 54.641 | 1.00 | 52.48 | A | C |
| ATOM | 1463 | OD1 | ASP | A | 203 | 51.198 | 45.506 | 53.400 | 1.00 | 54.33 | A | O |
| ATOM | 1464 | OD2 | ASP | A | 203 | 51.649 | 46.582 | 55.262 | 1.00 | 54.43 | A | O |
| ATOM | 1465 | C | ASP | A | 203 | 48.704 | 42.728 | 55.406 | 1.00 | 46.31 | A | C |
| ATOM | 1466 | O | ASP | A | 203 | 49.098 | 41.569 | 55.267 | 1.00 | 47.60 | A | O |
| ATOM | 1467 | N | ALA | A | 204 | 47.762 | 43.081 | 56.285 | 1.00 | 45.44 | A | N |
| ATOM | 1468 | CA | ALA | A | 204 | 47.095 | 42.095 | 57.145 | 1.00 | 42.57 | A | C |
| ATOM | 1469 | CB | ALA | A | 204 | 46.089 | 42.781 | 58.046 | 1.00 | 42.82 | A | C |
| ATOM | 1470 | C | ALA | A | 204 | 46.384 | 41.076 | 56.263 | 1.00 | 43.41 | A | C |
| ATOM | 1471 | O | ALA | A | 204 | 46.327 | 39.883 | 56.579 | 1.00 | 39.53 | A | O |
| ATOM | 1472 | N | TYR | A | 205 | 45.836 | 41.571 | 55.156 | 1.00 | 45.11 | A | N |
| ATOM | 1473 | CA | TYR | A | 205 | 45.136 | 40.733 | 54.200 | 1.00 | 47.57 | A | C |
| ATOM | 1474 | CB | TYR | A | 205 | 44.519 | 41.580 | 53.087 | 1.00 | 46.40 | A | C |
| ATOM | 1475 | CG | TYR | A | 205 | 43.919 | 40.725 | 52.007 | 1.00 | 48.17 | A | C |
| ATOM | 1476 | CD1 | TYR | A | 205 | 42.755 | 39.999 | 52.242 | 1.00 | 50.37 | A | C |
| ATOM | 1477 | CE1 | TYR | A | 205 | 42.234 | 39.133 | 51.287 | 1.00 | 48.40 | A | C |
| ATOM | 1478 | CD2 | TYR | A | 205 | 44.553 | 40.572 | 50.777 | 1.00 | 50.35 | A | C |
| ATOM | 1479 | CE2 | TYR | A | 205 | 44.048 | 39.705 | 49.812 | 1.00 | 49.64 | A | C |
| ATOM | 1480 | CZ | TYR | A | 205 | 42.887 | 38.984 | 50.079 | 1.00 | 51.15 | A | C |
| ATOM | 1481 | OH | TYR | A | 205 | 42.399 | 38.087 | 49.161 | 1.00 | 50.00 | A | O |
| ATOM | 1482 | C | TYR | A | 205 | 46.087 | 39.719 | 53.574 | 1.00 | 48.72 | A | C |
| ATOM | 1483 | O | TYR | A | 205 | 45.842 | 38.516 | 53.648 | 1.00 | 48.49 | A | O |
| ATOM | 1484 | N | ASP | A | 206 | 47.167 | 40.210 | 52.962 | 1.00 | 49.75 | A | N |
| ATOM | 1485 | CA | ASP | A | 206 | 48.145 | 39.333 | 52.311 | 1.00 | 48.01 | A | C |
| ATOM | 1486 | CB | ASP | A | 206 | 49.380 | 40.127 | 51.857 | 1.00 | 47.35 | A | C |
| ATOM | 1487 | CG | ASP | A | 206 | 49.056 | 41.186 | 50.822 | 1.00 | 50.43 | A | C |
| ATOM | 1488 | OD1 | ASP | A | 206 | 48.218 | 40.927 | 49.935 | 1.00 | 51.12 | A | O |
| ATOM | 1489 | OD2 | ASP | A | 206 | 49.655 | 42.282 | 50.883 | 1.00 | 54.15 | A | O |
| ATOM | 1490 | C | ASP | A | 206 | 48.587 | 38.200 | 53.237 | 1.00 | 45.61 | A | C |
| ATOM | 1491 | O | ASP | A | 206 | 48.673 | 37.048 | 52.822 | 1.00 | 45.44 | A | O |
| ATOM | 1492 | N | MET | A | 207 | 48.853 | 38.530 | 54.495 | 1.00 | 43.87 | A | N |
| ATOM | 1493 | CA | MET | A | 207 | 49.290 | 37.530 | 55.461 | 1.00 | 46.51 | A | C |
| ATOM | 1494 | CB | MET | A | 207 | 49.690 | 38.189 | 56.778 | 1.00 | 45.12 | A | C |
| ATOM | 1495 | CG | MET | A | 207 | 50.379 | 37.243 | 57.746 | 1.00 | 54.40 | A | C |
| ATOM | 1496 | SD | MET | A | 207 | 51.980 | 36.609 | 57.112 | 1.00 | 62.89 | A | S |
| ATOM | 1497 | CE | MET | A | 207 | 51.696 | 34.864 | 57.180 | 1.00 | 56.93 | A | C |
| ATOM | 1498 | C | MET | A | 207 | 48.221 | 36.489 | 55.736 | 1.00 | 48.58 | A | C |
| ATOM | 1499 | O | MET | A | 207 | 48.508 | 35.296 | 55.760 | 1.00 | 50.39 | A | O |
| ATOM | 1500 | N | ALA | A | 208 | 46.993 | 36.946 | 55.972 | 1.00 | 48.49 | A | N |
| ATOM | 1501 | CA | ALA | A | 208 | 45.897 | 36.033 | 56.238 | 1.00 | 47.38 | A | C |
| ATOM | 1502 | CB | ALA | A | 208 | 44.626 | 36.811 | 56.567 | 1.00 | 46.13 | A | C |
| ATOM | 1503 | C | ALA | A | 208 | 45.687 | 35.165 | 54.998 | 1.00 | 45.19 | A | C |
| ATOM | 1504 | O | ALA | A | 208 | 45.524 | 33.959 | 55.119 | 1.00 | 44.33 | A | O |
| ATOM | 1505 | N | LYS | A | 209 | 45.705 | 35.785 | 53.819 | 1.00 | 45.90 | A | N |
| ATOM | 1506 | CA | LYS | A | 209 | 45.524 | 35.081 | 52.541 | 1.00 | 52.96 | A | C |
| ATOM | 1507 | CB | LYS | A | 209 | 45.719 | 36.051 | 51.375 | 1.00 | 53.11 | A | C |
| ATOM | 1508 | CG | LYS | A | 209 | 45.509 | 35.434 | 49.997 | 1.00 | 54.69 | A | C |
| ATOM | 1509 | CD | LYS | A | 209 | 45.733 | 36.470 | 48.893 | 1.00 | 55.07 | A | C |
| ATOM | 1510 | CE | LYS | A | 209 | 45.352 | 35.925 | 47.521 | 1.00 | 57.02 | A | C |
| ATOM | 1511 | NZ | LYS | A | 209 | 46.049 | 34.639 | 47.242 | 1.00 | 60.76 | A | N |
| ATOM | 1512 | C | LYS | A | 209 | 46.511 | 33.922 | 52.387 | 1.00 | 56.68 | A | C |
| ATOM | 1513 | O | LYS | A | 209 | 46.135 | 32.806 | 52.023 | 1.00 | 55.80 | A | O |
| ATOM | 1514 | N | LEU | A | 210 | 47.778 | 34.209 | 52.664 | 1.00 | 61.39 | A | N |
| ATOM | 1515 | CA | LEU | A | 210 | 48.842 | 33.222 | 52.577 | 1.00 | 63.75 | A | C |
| ATOM | 1516 | CB | LEU | A | 210 | 50.171 | 33.870 | 52.970 | 1.00 | 68.80 | A | C |
| ATOM | 1517 | CG | LEU | A | 210 | 51.475 | 33.104 | 52.752 | 1.00 | 73.97 | A | C |
| ATOM | 1518 | CD1 | LEU | A | 210 | 52.642 | 34.061 | 52.951 | 1.00 | 78.93 | A | C |
| ATOM | 1519 | CD2 | LEU | A | 210 | 51.575 | 31.932 | 53.711 | 1.00 | 75.97 | A | C |
| ATOM | 1520 | C | LEU | A | 210 | 48.543 | 32.026 | 53.476 | 1.00 | 61.87 | A | C |
| ATOM | 1521 | O | LEU | A | 210 | 48.735 | 30.883 | 53.067 | 1.00 | 62.42 | A | O |
| ATOM | 1522 | N | LEU | A | 211 | 48.064 | 32.282 | 54.692 | 1.00 | 58.32 | A | N |
| ATOM | 1523 | CA | LEU | A | 211 | 47.747 | 31.193 | 55.621 | 1.00 | 56.55 | A | C |
| ATOM | 1524 | CB | LEU | A | 211 | 47.503 | 31.728 | 57.026 | 1.00 | 51.96 | A | C |
| ATOM | 1525 | CG | LEU | A | 211 | 48.626 | 32.435 | 57.759 | 1.00 | 48.45 | A | C |
| ATOM | 1526 | CD1 | LEU | A | 211 | 48.079 | 32.876 | 59.104 | 1.00 | 40.60 | A | C |
| ATOM | 1527 | CD2 | LEU | A | 211 | 49.828 | 31.501 | 57.927 | 1.00 | 48.57 | A | C |
| ATOM | 1528 | C | LEU | A | 211 | 46.499 | 30.446 | 55.189 | 1.00 | 57.28 | A | C |
| ATOM | 1529 | O | LEU | A | 211 | 46.383 | 29.231 | 55.362 | 1.00 | 57.94 | A | O |
| ATOM | 1530 | N | CYS | A | 212 | 45.550 | 31.199 | 54.654 | 1.00 | 58.73 | A | N |
| ATOM | 1531 | CA | CYS | A | 212 | 44.290 | 30.644 | 54.203 | 1.00 | 58.61 | A | C |
| ATOM | 1532 | CB | CYS | A | 212 | 43.341 | 31.783 | 53.820 | 1.00 | 56.11 | A | C |
| ATOM | 1533 | SG | CYS | A | 212 | 41.688 | 31.273 | 53.366 | 1.00 | 57.09 | A | S |
| ATOM | 1534 | C | CYS | A | 212 | 44.561 | 29.742 | 53.008 | 1.00 | 58.04 | A | C |
| ATOM | 1535 | O | CYS | A | 212 | 43.979 | 28.666 | 52.886 | 1.00 | 55.17 | A | O |
| ATOM | 1536 | N | ASP | A | 213 | 45.456 | 30.188 | 52.131 | 1.00 | 59.86 | A | N |
| ATOM | 1537 | CA | ASP | A | 213 | 45.808 | 29.413 | 50.950 | 1.00 | 59.51 | A | C |
| ATOM | 1538 | CB | ASP | A | 213 | 46.718 | 30.225 | 50.031 | 1.00 | 55.80 | A | C |
| ATOM | 1539 | CG | ASP | A | 213 | 45.944 | 31.203 | 49.160 | 1.00 | 58.91 | A | C |
| ATOM | 1540 | OD1 | ASP | A | 213 | 44.737 | 30.965 | 48.931 | 1.00 | 60.96 | A | O |
| ATOM | 1541 | OD2 | ASP | A | 213 | 46.536 | 32.200 | 48.683 | 1.00 | 58.55 | A | O |
| ATOM | 1542 | C | ASP | A | 213 | 46.475 | 28.092 | 51.325 | 1.00 | 61.01 | A | C |
| ATOM | 1543 | O | ASP | A | 213 | 46.158 | 27.062 | 50.753 | 1.00 | 60.88 | A | O |
| ATOM | 1544 | N | LYS | A | 214 | 47.390 | 28.113 | 52.289 | 1.00 | 65.17 | A | N |
| ATOM | 1545 | CA | LYS | A | 214 | 48.061 | 26.885 | 52.704 | 1.00 | 69.73 | A | C |
| ATOM | 1546 | CB | LYS | A | 214 | 49.149 | 27.166 | 53.748 | 1.00 | 73.76 | A | C |
| ATOM | 1547 | CG | LYS | A | 214 | 50.549 | 27.370 | 53.192 | 1.00 | 80.78 | A | C |
| ATOM | 1548 | CD | LYS | A | 214 | 51.593 | 27.059 | 54.257 | 1.00 | 87.75 | A | C |
| ATOM | 1549 | CE | LYS | A | 214 | 51.760 | 25.552 | 54.453 | 1.00 | 92.98 | A | C |
| ATOM | 1550 | NZ | LYS | A | 214 | 52.539 | 25.192 | 55.674 | 1.00 | 93.75 | A | N |
| ATOM | 1551 | C | LYS | A | 214 | 47.093 | 25.875 | 53.303 | 1.00 | 70.42 | A | C |
| ATOM | 1552 | O | LYS | A | 214 | 47.087 | 24.709 | 52.926 | 1.00 | 71.37 | A | O |
| ATOM | 1553 | N | TYR | A | 215 | 46.274 | 26.337 | 54.238 | 1.00 | 71.78 | A | N |
| ATOM | 1554 | CA | TYR | A | 215 | 45.336 | 25.475 | 54.937 | 1.00 | 71.94 | A | C |
| ATOM | 1555 | CB | TYR | A | 215 | 44.945 | 26.146 | 56.258 | 1.00 | 78.26 | A | C |
| ATOM | 1556 | CG | TYR | A | 215 | 44.167 | 25.270 | 57.211 | 1.00 | 85.38 | A | C |
| ATOM | 1557 | CD1 | TYR | A | 215 | 42.801 | 25.049 | 57.028 | 1.00 | 88.50 | A | C |
| ATOM | 1558 | CE1 | TYR | A | 215 | 42.081 | 24.228 | 57.887 | 1.00 | 90.88 | A | C |
| ATOM | 1559 | CD2 | TYR | A | 215 | 44.797 | 24.646 | 58.287 | 1.00 | 88.11 | A | C |
| ATOM | 1560 | CE2 | TYR | A | 215 | 44.086 | 23.820 | 59.155 | 1.00 | 91.46 | A | C |
| ATOM | 1561 | CZ | TYR | A | 215 | 42.727 | 23.615 | 58.947 | 1.00 | 92.33 | A | C |
| ATOM | 1562 | OH | TYR | A | 215 | 42.007 | 22.790 | 59.780 | 1.00 | 92.08 | A | O |
| ATOM | 1563 | C | TYR | A | 215 | 44.092 | 25.059 | 54.153 | 1.00 | 70.47 | A | C |
| ATOM | 1564 | O | TYR | A | 215 | 43.633 | 23.928 | 54.281 | 1.00 | 69.61 | A | O |
| ATOM | 1565 | N | TYR | A | 216 | 43.545 | 25.954 | 53.338 | 1.00 | 70.09 | A | N |
| ATOM | 1566 | CA | TYR | A | 216 | 42.349 | 25.619 | 52.571 | 1.00 | 69.02 | A | C |
| ATOM | 1567 | CB | TYR | A | 216 | 41.230 | 26.627 | 52.844 | 1.00 | 66.23 | A | C |
| ATOM | 1568 | CG | TYR | A | 216 | 40.759 | 26.683 | 54.280 | 1.00 | 64.01 | A | C |
| ATOM | 1569 | CD1 | TYR | A | 216 | 41.216 | 27.675 | 55.145 | 1.00 | 61.32 | A | C |
| ATOM | 1570 | CE1 | TYR | A | 216 | 40.756 | 27.753 | 56.461 | 1.00 | 61.88 | A | C |
| ATOM | 1571 | CD2 | TYR | A | 216 | 39.833 | 25.761 | 54.769 | 1.00 | 62.28 | A | C |
| ATOM | 1572 | CE2 | TYR | A | 216 | 39.370 | 25.828 | 56.083 | 1.00 | 61.82 | A | C |
| ATOM | 1573 | CZ | TYR | A | 216 | 39.832 | 26.827 | 56.923 | 1.00 | 62.52 | A | C |
| ATOM | 1574 | OH | TYR | A | 216 | 39.372 | 26.910 | 58.221 | 1.00 | 62.08 | A | O |
| ATOM | 1575 | C | TYR | A | 216 | 42.580 | 25.544 | 51.069 | 1.00 | 72.54 | A | C |
| ATOM | 1576 | O | TYR | A | 216 | 41.742 | 25.021 | 50.340 | 1.00 | 73.12 | A | O |
| ATOM | 1577 | N | MET | A | 217 | 43.699 | 26.082 | 50.600 | 1.00 | 76.33 | A | N |
| ATOM | 1578 | CA | MET | A | 217 | 44.015 | 26.048 | 49.175 | 1.00 | 82.55 | A | C |
| ATOM | 1579 | CB | MET | A | 217 | 44.217 | 24.590 | 48.748 | 1.00 | 90.55 | A | C |
| ATOM | 1580 | CG | MET | A | 217 | 45.331 | 23.914 | 49.554 | 1.00 | 100.88 | A | C |
| ATOM | 1581 | SD | MET | A | 217 | 45.312 | 22.114 | 49.562 | 1.00 | 109.24 | A | S |
| ATOM | 1582 | CE | MET | A | 217 | 44.184 | 21.787 | 50.922 | 1.00 | 112.86 | A | C |
| ATOM | 1583 | C | MET | A | 217 | 42.945 | 26.743 | 48.327 | 1.00 | 81.23 | A | C |
| ATOM | 1584 | O | MET | A | 217 | 42.622 | 26.329 | 47.208 | 1.00 | 82.72 | A | O |
| ATOM | 1585 | N | ALA | A | 218 | 42.411 | 27.818 | 48.895 | 1.00 | 77.25 | A | N |
| ATOM | 1586 | CA | ALA | A | 218 | 41.395 | 28.655 | 48.271 | 1.00 | 70.53 | A | C |
| ATOM | 1587 | CB | ALA | A | 218 | 40.094 | 27.887 | 48.097 | 1.00 | 71.10 | A | C |
| ATOM | 1588 | C | ALA | A | 218 | 41.218 | 29.816 | 49.245 | 1.00 | 65.10 | A | C |
| ATOM | 1589 | O | ALA | A | 218 | 41.317 | 29.628 | 50.458 | 1.00 | 63.41 | A | O |
| ATOM | 1590 | N | SER | A | 219 | 40.986 | 31.015 | 48.724 | 1.00 | 60.39 | A | N |
| ATOM | 1591 | CA | SER | A | 219 | 40.836 | 32.180 | 49.583 | 1.00 | 55.27 | A | C |
| ATOM | 1592 | CB | SER | A | 219 | 42.208 | 32.713 | 49.997 | 1.00 | 55.43 | A | C |
| ATOM | 1593 | OG | SER | A | 219 | 42.803 | 33.434 | 48.926 | 1.00 | 55.80 | A | O |
| ATOM | 1594 | C | SER | A | 219 | 40.090 | 33.277 | 48.850 | 1.00 | 54.32 | A | C |
| ATOM | 1595 | O | SER | A | 219 | 40.230 | 33.429 | 47.634 | 1.00 | 53.74 | A | O |
| ATOM | 1596 | N | PRO | A | 220 | 39.285 | 34.064 | 49.584 | 1.00 | 52.77 | A | N |
| ATOM | 1597 | CD | PRO | A | 220 | 38.937 | 33.901 | 51.007 | 1.00 | 48.99 | A | C |
| ATOM | 1598 | CA | PRO | A | 220 | 38.521 | 35.157 | 48.973 | 1.00 | 49.92 | A | C |
| ATOM | 1599 | CB | PRO | A | 220 | 37.484 | 35.486 | 50.045 | 1.00 | 50.39 | A | C |
| ATOM | 1600 | CG | PRO | A | 220 | 38.220 | 35.196 | 51.313 | 1.00 | 50.59 | A | C |
| ATOM | 1601 | C | PRO | A | 220 | 39.426 | 36.338 | 48.655 | 1.00 | 49.50 | A | C |
| ATOM | 1602 | O | PRO | A | 220 | 40.479 | 36.485 | 49.255 | 1.00 | 48.82 | A | O |
| ATOM | 1603 | N | ASP | A | 221 | 39.014 | 37.175 | 47.712 | 1.00 | 48.94 | A | N |
| ATOM | 1604 | CA | ASP | A | 221 | 39.796 | 38.342 | 47.344 | 1.00 | 49.87 | A | C |
| ATOM | 1605 | CB | ASP | A | 221 | 39.522 | 38.710 | 45.888 | 1.00 | 52.82 | A | C |
| ATOM | 1606 | CG | ASP | A | 221 | 39.829 | 37.568 | 44.938 | 1.00 | 57.28 | A | C |
| ATOM | 1607 | OD1 | ASP | A | 221 | 40.909 | 36.945 | 45.077 | 1.00 | 57.98 | A | O |
| ATOM | 1608 | OD2 | ASP | A | 221 | 38.992 | 37.292 | 44.054 | 1.00 | 60.25 | A | O |
| ATOM | 1609 | C | ASP | A | 221 | 39.482 | 39.534 | 48.252 | 1.00 | 51.44 | A | C |
| ATOM | 1610 | O | ASP | A | 221 | 38.715 | 39.419 | 49.208 | 1.00 | 49.18 | A | O |
| ATOM | 1611 | N | LEU | A | 222 | 40.065 | 40.683 | 47.938 | 1.00 | 48.48 | A | N |
| ATOM | 1612 | CA | LEU | A | 222 | 39.847 | 41.867 | 48.740 | 1.00 | 49.51 | A | C |
| ATOM | 1613 | CB | LEU | A | 222 | 41.101 | 42.198 | 49.564 | 1.00 | 47.83 | A | C |
| ATOM | 1614 | CG | LEU | A | 222 | 41.116 | 43.575 | 50.251 | 1.00 | 46.71 | A | C |
| ATOM | 1615 | CD1 | LEU | A | 222 | 40.221 | 43.544 | 51.485 | 1.00 | 44.44 | A | C |
| ATOM | 1616 | CD2 | LEU | A | 222 | 42.542 | 43.953 | 50.637 | 1.00 | 46.17 | A | C |
| ATOM | 1617 | C | LEU | A | 222 | 39.477 | 43.090 | 47.924 | 1.00 | 49.54 | A | C |
| ATOM | 1618 | O | LEU | A | 222 | 40.063 | 43.366 | 46.876 | 1.00 | 54.05 | A | O |
| ATOM | 1619 | N | GLU | A | 223 | 38.492 | 43.819 | 48.421 | 1.00 | 46.46 | A | N |
| ATOM | 1620 | CA | GLU | A | 223 | 38.077 | 45.054 | 47.801 | 1.00 | 44.96 | A | C |
| ATOM | 1621 | CB | GLU | A | 223 | 36.635 | 44.960 | 47.313 | 1.00 | 46.96 | A | C |
| ATOM | 1622 | CG | GLU | A | 223 | 36.494 | 44.196 | 46.004 | 1.00 | 53.60 | A | C |
| ATOM | 1623 | CD | GLU | A | 223 | 35.047 | 44.004 | 45.579 | 1.00 | 58.10 | A | C |
| ATOM | 1624 | OE1 | GLU | A | 223 | 34.252 | 44.972 | 45.670 | 1.00 | 58.61 | A | O |
| ATOM | 1625 | OE2 | GLU | A | 223 | 34.713 | 42.879 | 45.146 | 1.00 | 60.96 | A | O |
| ATOM | 1626 | C | GLU | A | 223 | 38.199 | 46.061 | 48.924 | 1.00 | 43.00 | A | C |
| ATOM | 1627 | O | GLU | A | 223 | 37.851 | 45.761 | 50.065 | 1.00 | 44.80 | A | O |
| ATOM | 1628 | N | ILE | A | 224 | 38.737 | 47.231 | 48.618 | 1.00 | 40.14 | A | N |
| ATOM | 1629 | CA | ILE | A | 224 | 38.873 | 48.273 | 49.612 | 1.00 | 40.03 | A | C |
| ATOM | 1630 | CB | ILE | A | 224 | 40.346 | 48.601 | 49.939 | 1.00 | 39.41 | A | C |
| ATOM | 1631 | CG2 | ILE | A | 224 | 40.419 | 49.802 | 50.898 | 1.00 | 37.27 | A | C |
| ATOM | 1632 | CG1 | ILE | A | 224 | 41.021 | 47.388 | 50.570 | 1.00 | 41.70 | A | C |
| ATOM | 1633 | CD1 | ILE | A | 224 | 42.468 | 47.627 | 50.939 | 1.00 | 45.81 | A | C |
| ATOM | 1634 | C | ILE | A | 224 | 38.250 | 49.493 | 49.000 | 1.00 | 41.46 | A | C |
| ATOM | 1635 | O | ILE | A | 224 | 38.237 | 49.638 | 47.789 | 1.00 | 44.53 | A | O |
| ATOM | 1636 | N | GLN | A | 225 | 37.729 | 50.371 | 49.843 | 1.00 | 44.57 | A | N |
| ATOM | 1637 | CA | GLN | A | 225 | 37.107 | 51.591 | 49.379 | 1.00 | 44.30 | A | C |
| ATOM | 1638 | CB | GLN | A | 225 | 35.610 | 51.376 | 49.156 | 1.00 | 45.78 | A | C |
| ATOM | 1639 | CG | GLN | A | 225 | 34.928 | 52.549 | 48.467 | 1.00 | 60.36 | A | C |
| ATOM | 1640 | CD | GLN | A | 225 | 33.419 | 52.411 | 48.423 | 1.00 | 65.93 | A | C |
| ATOM | 1641 | OE1 | GLN | A | 225 | 32.886 | 51.330 | 48.147 | 1.00 | 69.14 | A | O |
| ATOM | 1642 | NE2 | GLN | A | 225 | 32.718 | 53.510 | 48.683 | 1.00 | 69.13 | A | N |
| ATOM | 1643 | C | GLN | A | 225 | 37.318 | 52.609 | 50.474 | 1.00 | 41.39 | A | C |
| ATOM | 1644 | O | GLN | A | 225 | 37.135 | 52.305 | 51.645 | 1.00 | 42.72 | A | O |
| ATOM | 1645 | N | GLU | A | 226 | 37.701 | 53.814 | 50.089 | 1.00 | 41.74 | A | N |
| ATOM | 1646 | CA | GLU | A | 226 | 37.929 | 54.872 | 51.048 | 1.00 | 45.37 | A | C |
| ATOM | 1647 | CB | GLU | A | 226 | 39.312 | 55.474 | 50.835 | 1.00 | 45.72 | A | C |
| ATOM | 1648 | CG | GLU | A | 226 | 40.401 | 54.448 | 50.863 | 1.00 | 49.41 | A | C |
| ATOM | 1649 | CD | GLU | A | 226 | 41.729 | 55.027 | 51.295 | 1.00 | 56.74 | A | C |
| ATOM | 1650 | OE1 | GLU | A | 226 | 42.577 | 54.236 | 51.754 | 1.00 | 60.13 | A | O |
| ATOM | 1651 | OE2 | GLU | A | 226 | 41.938 | 56.258 | 51.173 | 1.00 | 54.11 | A | O |
| ATOM | 1652 | C | GLU | A | 226 | 36.879 | 55.960 | 50.912 | 1.00 | 44.76 | A | C |
| ATOM | 1653 | O | GLU | A | 226 | 36.367 | 56.198 | 49.830 | 1.00 | 44.21 | A | O |
| ATOM | 1654 | N | ILE | A | 227 | 36.559 | 56.603 | 52.033 | 1.00 | 46.65 | A | N |
| ATOM | 1655 | CA | ILE | A | 227 | 35.598 | 57.693 | 52.074 | 1.00 | 45.36 | A | C |
| ATOM | 1656 | CB | ILE | A | 227 | 34.268 | 57.295 | 52.763 | 1.00 | 46.31 | A | C |
| ATOM | 1657 | CG2 | ILE | A | 227 | 33.297 | 58.475 | 52.719 | 1.00 | 45.12 | A | C |
| ATOM | 1658 | CG1 | ILE | A | 227 | 33.666 | 56.054 | 52.099 | 1.00 | 46.37 | A | C |
| ATOM | 1659 | CD1 | ILE | A | 227 | 33.301 | 56.237 | 50.650 | 1.00 | 46.45 | A | C |
| ATOM | 1660 | C | ILE | A | 227 | 36.242 | 58.777 | 52.922 | 1.00 | 45.68 | A | C |
| ATOM | 1661 | O | ILE | A | 227 | 36.091 | 58.786 | 54.145 | 1.00 | 45.07 | A | O |
| ATOM | 1662 | N | ASN | A | 228 | 36.981 | 59.674 | 52.287 | 1.00 | 46.35 | A | N |
| ATOM | 1663 | CA | ASN | A | 228 | 37.616 | 60.758 | 53.019 | 1.00 | 47.93 | A | C |
| ATOM | 1664 | CB | ASN | A | 228 | 38.934 | 61.143 | 52.354 | 1.00 | 42.76 | A | C |
| ATOM | 1665 | CG | ASN | A | 228 | 39.729 | 62.121 | 53.176 | 1.00 | 43.96 | A | C |
| ATOM | 1666 | OD1 | ASN | A | 228 | 39.162 | 62.967 | 53.871 | 1.00 | 46.08 | A | O |
| ATOM | 1667 | ND2 | ASN | A | 228 | 41.050 | 62.029 | 53.094 | 1.00 | 42.61 | A | N |
| ATOM | 1668 | C | ASN | A | 228 | 36.632 | 61.921 | 52.965 | 1.00 | 50.88 | A | C |
| ATOM | 1669 | O | ASN | A | 228 | 36.711 | 62.772 | 52.087 | 1.00 | 53.66 | A | O |
| ATOM | 1670 | N | ALA | A | 229 | 35.701 | 61.941 | 53.912 | 1.00 | 52.59 | A | N |
| ATOM | 1671 | CA | ALA | A | 229 | 34.663 | 62.964 | 53.978 | 1.00 | 56.15 | A | C |
| ATOM | 1672 | CB | ALA | A | 229 | 34.006 | 62.937 | 55.358 | 1.00 | 54.38 | A | C |
| ATOM | 1673 | C | ALA | A | 229 | 35.070 | 64.402 | 53.625 | 1.00 | 59.02 | A | C |
| ATOM | 1674 | O | ALA | A | 229 | 34.517 | 64.993 | 52.698 | 1.00 | 59.28 | A | O |
| ATOM | 1675 | N | ALA | A | 230 | 36.024 | 64.959 | 54.365 | 1.00 | 62.53 | A | N |
| ATOM | 1676 | CA | ALA | A | 230 | 36.469 | 66.333 | 54.149 | 1.00 | 64.57 | A | C |
| ATOM | 1677 | CB | ALA | A | 230 | 37.243 | 66.819 | 55.374 | 1.00 | 62.98 | A | C |
| ATOM | 1678 | C | ALA | A | 230 | 37.291 | 66.576 | 52.884 | 1.00 | 66.31 | A | C |
| ATOM | 1679 | O | ALA | A | 230 | 37.190 | 67.644 | 52.286 | 1.00 | 67.76 | A | O |
| ATOM | 1680 | N | ASN | A | 231 | 38.107 | 65.603 | 52.484 | 1.00 | 68.55 | A | N |
| ATOM | 1681 | CA | ASN | A | 231 | 38.939 | 65.735 | 51.285 | 1.00 | 67.87 | A | C |
| ATOM | 1682 | CB | ASN | A | 231 | 40.413 | 65.862 | 51.672 | 1.00 | 72.22 | A | C |
| ATOM | 1683 | CG | ASN | A | 231 | 40.689 | 67.087 | 52.524 | 1.00 | 77.34 | A | C |
| ATOM | 1684 | OD1 | ASN | A | 231 | 40.235 | 67.180 | 53.666 | 1.00 | 79.16 | A | O |
| ATOM | 1685 | ND2 | ASN | A | 231 | 41.434 | 68.039 | 51.969 | 1.00 | 80.29 | A | N |
| ATOM | 1686 | C | ASN | A | 231 | 38.742 | 64.539 | 50.362 | 1.00 | 68.26 | A | C |
| ATOM | 1687 | O | ASN | A | 231 | 39.542 | 63.607 | 50.341 | 1.00 | 66.75 | A | O |
| ATOM | 1688 | N | SER | A | 232 | 37.664 | 64.600 | 49.591 | 1.00 | 71.02 | A | N |
| ATOM | 1689 | CA | SER | A | 232 | 37.254 | 63.561 | 48.652 | 1.00 | 75.13 | A | C |
| ATOM | 1690 | CB | SER | A | 232 | 36.489 | 64.212 | 47.497 | 1.00 | 76.44 | A | C |
| ATOM | 1691 | OG | SER | A | 232 | 35.902 | 63.228 | 46.662 | 1.00 | 77.22 | A | O |
| ATOM | 1692 | C | SER | A | 232 | 38.272 | 62.567 | 48.076 | 1.00 | 78.74 | A | C |
| ATOM | 1693 | O | SER | A | 232 | 37.968 | 61.381 | 47.968 | 1.00 | 78.13 | A | O |
| ATOM | 1694 | N | LYS | A | 233 | 39.463 | 63.008 | 47.689 | 1.00 | 84.59 | A | N |
| ATOM | 1695 | CA | LYS | A | 233 | 40.401 | 62.042 | 47.121 | 1.00 | 90.73 | A | C |
| ATOM | 1696 | CB | LYS | A | 233 | 40.612 | 62.318 | 45.631 | 1.00 | 98.55 | A | C |
| ATOM | 1697 | CG | LYS | A | 233 | 39.405 | 61.960 | 44.774 | 1.00 | 108.83 | A | C |
| ATOM | 1698 | CD | LYS | A | 233 | 38.864 | 60.582 | 45.148 | 1.00 | 116.61 | A | C |
| ATOM | 1699 | CE | LYS | A | 233 | 37.703 | 60.170 | 44.260 | 1.00 | 121.01 | A | C |
| ATOM | 1700 | NZ | LYS | A | 233 | 38.150 | 59.940 | 42.861 | 1.00 | 124.99 | A | N |
| ATOM | 1701 | C | LYS | A | 233 | 41.751 | 61.870 | 47.791 | 1.00 | 89.93 | A | C |
| ATOM | 1702 | O | LYS | A | 233 | 42.505 | 60.964 | 47.434 | 1.00 | 89.66 | A | O |
| ATOM | 1703 | N | GLN | A | 234 | 42.072 | 62.725 | 48.752 | 1.00 | 89.58 | A | N |
| ATOM | 1704 | CA | GLN | A | 234 | 43.348 | 62.582 | 49.429 | 1.00 | 89.21 | A | C |
| ATOM | 1705 | CB | GLN | A | 234 | 43.560 | 63.731 | 50.421 | 1.00 | 100.85 | A | C |
| ATOM | 1706 | CG | GLN | A | 234 | 43.556 | 65.122 | 49.780 | 1.00 | 119.19 | A | C |
| ATOM | 1707 | CD | GLN | A | 234 | 44.521 | 65.244 | 48.608 | 1.00 | 129.15 | A | C |
| ATOM | 1708 | OE1 | GLN | A | 234 | 44.327 | 64.623 | 47.562 | 1.00 | 134.81 | A | O |
| ATOM | 1709 | NE2 | GLN | A | 234 | 45.568 | 66.044 | 48.781 | 1.00 | 135.91 | A | N |
| ATOM | 1710 | C | GLN | A | 234 | 43.342 | 61.233 | 50.153 | 1.00 | 79.91 | A | C |
| ATOM | 1711 | O | GLN | A | 234 | 42.369 | 60.883 | 50.823 | 1.00 | 77.42 | A | O |
| ATOM | 1712 | N | PRO | A | 235 | 44.414 | 60.437 | 49.991 | 1.00 | 72.41 | A | N |
| ATOM | 1713 | CD | PRO | A | 235 | 45.595 | 60.650 | 49.136 | 1.00 | 69.70 | A | C |
| ATOM | 1714 | CA | PRO | A | 235 | 44.480 | 59.132 | 50.657 | 1.00 | 64.97 | A | C |
| ATOM | 1715 | CB | PRO | A | 235 | 45.882 | 58.646 | 50.318 | 1.00 | 65.24 | A | C |
| ATOM | 1716 | CG | PRO | A | 235 | 46.116 | 59.241 | 48.964 | 1.00 | 66.26 | A | C |
| ATOM | 1717 | C | PRO | A | 235 | 44.269 | 59.300 | 52.161 | 1.00 | 59.70 | A | C |
| ATOM | 1718 | O | PRO | A | 235 | 44.577 | 60.347 | 52.733 | 1.00 | 56.88 | A | O |
| ATOM | 1719 | N | ILE | A | 236 | 43.736 | 58.272 | 52.804 | 1.00 | 55.64 | A | N |
| ATOM | 1720 | CA | ILE | A | 236 | 43.483 | 58.359 | 54.228 | 1.00 | 51.18 | A | C |
| ATOM | 1721 | CB | ILE | A | 236 | 42.291 | 57.466 | 54.636 | 1.00 | 49.00 | A | C |
| ATOM | 1722 | CG2 | ILE | A | 236 | 42.125 | 57.474 | 56.154 | 1.00 | 42.93 | A | C |
| ATOM | 1723 | CG1 | ILE | A | 236 | 41.014 | 57.975 | 53.948 | 1.00 | 45.76 | A | C |
| ATOM | 1724 | CD1 | ILE | A | 236 | 39.782 | 57.108 | 54.159 | 1.00 | 42.22 | A | C |
| ATOM | 1725 | C | ILE | A | 236 | 44.707 | 57.993 | 55.052 | 1.00 | 49.88 | A | C |
| ATOM | 1726 | O | ILE | A | 236 | 45.180 | 56.862 | 55.011 | 1.00 | 47.00 | A | O |
| ATOM | 1727 | N | HIS | A | 237 | 45.205 | 58.979 | 55.794 | 1.00 | 52.18 | A | N |
| ATOM | 1728 | CA | HIS | A | 237 | 46.367 | 58.830 | 56.666 | 1.00 | 54.36 | A | C |
| ATOM | 1729 | CB | HIS | A | 237 | 47.506 | 59.773 | 56.236 | 1.00 | 62.14 | A | C |
| ATOM | 1730 | CG | HIS | A | 237 | 48.233 | 59.336 | 55.001 | 1.00 | 72.17 | A | C |
| ATOM | 1731 | CD2 | HIS | A | 237 | 49.476 | 58.827 | 54.837 | 1.00 | 77.38 | A | C |
| ATOM | 1732 | ND1 | HIS | A | 237 | 47.674 | 59.392 | 53.742 | 1.00 | 77.51 | A | N |
| ATOM | 1733 | CE1 | HIS | A | 237 | 48.541 | 58.935 | 52.856 | 1.00 | 78.58 | A | C |
| ATOM | 1734 | NE2 | HIS | A | 237 | 49.642 | 58.585 | 53.494 | 1.00 | 79.31 | A | N |
| ATOM | 1735 | C | HIS | A | 237 | 45.943 | 59.202 | 58.079 | 1.00 | 50.50 | A | C |
| ATOM | 1736 | O | HIS | A | 237 | 44.981 | 59.945 | 58.274 | 1.00 | 49.94 | A | O |
| ATOM | 1737 | N | MET | A | 238 | 46.670 | 58.695 | 59.064 | 1.00 | 46.61 | A | N |
| ATOM | 1738 | CA | MET | A | 238 | 46.378 | 58.999 | 60.455 | 1.00 | 42.81 | A | C |
| ATOM | 1739 | CB | MET | A | 238 | 45.293 | 58.066 | 61.008 | 1.00 | 43.11 | A | C |
| ATOM | 1740 | CG | MET | A | 238 | 45.771 | 56.637 | 61.348 | 1.00 | 37.78 | A | C |
| ATOM | 1741 | SD | MET | A | 238 | 44.479 | 55.613 | 62.133 | 1.00 | 42.83 | A | S |
| ATOM | 1742 | CE | MET | A | 238 | 44.063 | 56.635 | 63.596 | 1.00 | 36.72 | A | C |
| ATOM | 1743 | C | MET | A | 238 | 47.650 | 58.780 | 61.244 | 1.00 | 43.88 | A | C |
| ATOM | 1744 | O | MET | A | 238 | 48.609 | 58.167 | 60.751 | 1.00 | 43.29 | A | O |
| ATOM | 1745 | N | VAL | A | 239 | 47.656 | 59.284 | 62.469 | 1.00 | 43.14 | A | N |
| ATOM | 1746 | CA | VAL | A | 239 | 48.789 | 59.108 | 63.354 | 1.00 | 43.62 | A | C |
| ATOM | 1747 | CB | VAL | A | 239 | 48.996 | 60.350 | 64.262 | 1.00 | 44.61 | A | C |
| ATOM | 1748 | CG1 | VAL | A | 239 | 50.131 | 60.097 | 65.259 | 1.00 | 41.77 | A | C |
| ATOM | 1749 | CG2 | VAL | A | 239 | 49.288 | 61.577 | 63.410 | 1.00 | 42.14 | A | C |
| ATOM | 1750 | C | VAL | A | 239 | 48.390 | 57.917 | 64.224 | 1.00 | 43.90 | A | C |
| ATOM | 1751 | O | VAL | A | 239 | 47.429 | 58.005 | 65.004 | 1.00 | 42.67 | A | O |
| ATOM | 1752 | N | TYR | A | 240 | 49.100 | 56.801 | 64.097 | 1.00 | 39.99 | A | N |
| ATOM | 1753 | CA | TYR | A | 240 | 48.749 | 55.644 | 64.916 | 1.00 | 41.54 | A | C |
| ATOM | 1754 | CB | TYR | A | 240 | 47.552 | 54.912 | 64.283 | 1.00 | 38.04 | A | C |
| ATOM | 1755 | CG | TYR | A | 240 | 46.756 | 54.065 | 65.260 | 1.00 | 37.85 | A | C |
| ATOM | 1756 | CD1 | TYR | A | 240 | 46.790 | 52.661 | 65.201 | 1.00 | 32.68 | A | C |
| ATOM | 1757 | CE1 | TYR | A | 240 | 46.061 | 51.883 | 66.099 | 1.00 | 30.10 | A | C |
| ATOM | 1758 | CD2 | TYR | A | 240 | 45.973 | 54.664 | 66.248 | 1.00 | 30.15 | A | C |
| ATOM | 1759 | CE2 | TYR | A | 240 | 45.245 | 53.894 | 67.155 | 1.00 | 33.46 | A | C |
| ATOM | 1760 | CZ | TYR | A | 240 | 45.294 | 52.509 | 67.076 | 1.00 | 33.93 | A | C |
| ATOM | 1761 | OH | TYR | A | 240 | 44.578 | 51.767 | 67.983 | 1.00 | 32.67 | A | O |
| ATOM | 1762 | C | TYR | A | 240 | 49.922 | 54.688 | 65.072 | 1.00 | 42.33 | A | C |
| ATOM | 1763 | O | TYR | A | 240 | 51.021 | 54.959 | 64.573 | 1.00 | 44.60 | A | O |
| ATOM | 1764 | N | VAL | A | 241 | 49.686 | 53.586 | 65.786 | 1.00 | 41.59 | A | N |
| ATOM | 1765 | CA | VAL | A | 241 | 50.696 | 52.543 | 65.992 | 1.00 | 38.88 | A | C |
| ATOM | 1766 | CB | VAL | A | 241 | 50.617 | 51.961 | 67.411 | 1.00 | 38.96 | A | C |
| ATOM | 1767 | CG1 | VAL | A | 241 | 51.810 | 51.046 | 67.652 | 1.00 | 38.72 | A | C |
| ATOM | 1768 | CG2 | VAL | A | 241 | 50.563 | 53.084 | 68.445 | 1.00 | 39.22 | A | C |
| ATOM | 1769 | C | VAL | A | 241 | 50.319 | 51.435 | 64.989 | 1.00 | 37.77 | A | C |
| ATOM | 1770 | O | VAL | A | 241 | 49.446 | 50.596 | 65.273 | 1.00 | 36.01 | A | O |
| ATOM | 1771 | N | PRO | A | 242 | 50.970 | 51.412 | 63.812 | 1.00 | 36.25 | A | N |
| ATOM | 1772 | CD | PRO | A | 242 | 52.048 | 52.303 | 63.347 | 1.00 | 36.56 | A | C |
| ATOM | 1773 | CA | PRO | A | 242 | 50.653 | 50.397 | 62.799 | 1.00 | 38.50 | A | C |
| ATOM | 1774 | CB | PRO | A | 242 | 51.692 | 50.656 | 61.695 | 1.00 | 38.04 | A | C |
| ATOM | 1775 | CG | PRO | A | 242 | 52.795 | 51.410 | 62.391 | 1.00 | 36.62 | A | C |
| ATOM | 1776 | C | PRO | A | 242 | 50.586 | 48.939 | 63.246 | 1.00 | 38.13 | A | C |
| ATOM | 1777 | O | PRO | A | 242 | 49.653 | 48.223 | 62.885 | 1.00 | 36.76 | A | O |
| ATOM | 1778 | N | SER | A | 243 | 51.543 | 48.499 | 64.050 | 1.00 | 39.74 | A | N |
| ATOM | 1779 | CA | SER | A | 243 | 51.526 | 47.115 | 64.496 | 1.00 | 42.76 | A | C |
| ATOM | 1780 | CB | SER | A | 243 | 52.773 | 46.810 | 65.348 | 1.00 | 42.49 | A | C |
| ATOM | 1781 | OG | SER | A | 243 | 52.606 | 47.210 | 66.695 | 1.00 | 42.80 | A | O |
| ATOM | 1782 | C | SER | A | 243 | 50.242 | 46.793 | 65.283 | 1.00 | 42.50 | A | C |
| ATOM | 1783 | O | SER | A | 243 | 49.745 | 45.664 | 65.230 | 1.00 | 42.14 | A | O |
| ATOM | 1784 | N | HIS | A | 244 | 49.709 | 47.771 | 66.015 | 1.00 | 39.14 | A | N |
| ATOM | 1785 | CA | HIS | A | 244 | 48.475 | 47.552 | 66.779 | 1.00 | 40.52 | A | C |
| ATOM | 1786 | CB | HIS | A | 244 | 48.163 | 48.749 | 67.667 | 1.00 | 39.05 | A | C |
| ATOM | 1787 | CG | HIS | A | 244 | 48.962 | 48.802 | 68.925 | 1.00 | 45.05 | A | C |
| ATOM | 1788 | CD2 | HIS | A | 244 | 48.719 | 49.424 | 70.105 | 1.00 | 43.88 | A | C |
| ATOM | 1789 | ND1 | HIS | A | 244 | 50.206 | 48.220 | 69.048 | 1.00 | 48.43 | A | N |
| ATOM | 1790 | CE1 | HIS | A | 244 | 50.694 | 48.481 | 70.248 | 1.00 | 45.95 | A | C |
| ATOM | 1791 | NE2 | HIS | A | 244 | 49.811 | 49.211 | 70.908 | 1.00 | 45.33 | A | N |
| ATOM | 1792 | C | HIS | A | 244 | 47.302 | 47.352 | 65.832 | 1.00 | 39.79 | A | C |
| ATOM | 1793 | O | HIS | A | 244 | 46.458 | 46.474 | 66.040 | 1.00 | 38.99 | A | O |
| ATOM | 1794 | N | LEU | A | 245 | 47.262 | 48.182 | 64.792 | 1.00 | 38.80 | A | N |
| ATOM | 1795 | CA | LEU | A | 245 | 46.201 | 48.143 | 63.797 | 1.00 | 40.22 | A | C |
| ATOM | 1796 | CB | LEU | A | 245 | 46.287 | 49.391 | 62.902 | 1.00 | 38.36 | A | C |
| ATOM | 1797 | CG | LEU | A | 245 | 45.257 | 49.435 | 61.774 | 1.00 | 40.08 | A | C |
| ATOM | 1798 | CD1 | LEU | A | 245 | 43.866 | 49.381 | 62.381 | 1.00 | 36.82 | A | C |
| ATOM | 1799 | CD2 | LEU | A | 245 | 45.444 | 50.682 | 60.924 | 1.00 | 35.01 | A | C |
| ATOM | 1800 | C | LEU | A | 245 | 46.299 | 46.866 | 62.959 | 1.00 | 41.56 | A | C |
| ATOM | 1801 | O | LEU | A | 245 | 45.280 | 46.264 | 62.607 | 1.00 | 38.78 | A | O |
| ATOM | 1802 | N | TYR | A | 246 | 47.528 | 46.459 | 62.649 | 1.00 | 43.93 | A | N |
| ATOM | 1803 | CA | TYR | A | 246 | 47.779 | 45.237 | 61.878 | 1.00 | 46.13 | A | C |
| ATOM | 1804 | CB | TYR | A | 246 | 49.286 | 45.039 | 61.647 | 1.00 | 47.36 | A | C |
| ATOM | 1805 | CG | TYR | A | 246 | 49.642 | 43.758 | 60.909 | 1.00 | 48.18 | A | C |
| ATOM | 1806 | CD1 | TYR | A | 246 | 49.908 | 43.761 | 59.536 | 1.00 | 46.55 | A | C |
| ATOM | 1807 | CE1 | TYR | A | 246 | 50.230 | 42.576 | 58.861 | 1.00 | 48.92 | A | C |
| ATOM | 1808 | CD2 | TYR | A | 246 | 49.706 | 42.540 | 61.587 | 1.00 | 48.70 | A | C |
| ATOM | 1809 | CE2 | TYR | A | 246 | 50.022 | 41.355 | 60.925 | 1.00 | 47.11 | A | C |
| ATOM | 1810 | CZ | TYR | A | 246 | 50.283 | 41.375 | 59.572 | 1.00 | 49.54 | A | C |
| ATOM | 1811 | OH | TYR | A | 246 | 50.581 | 40.185 | 58.946 | 1.00 | 51.53 | A | O |
| ATOM | 1812 | C | TYR | A | 246 | 47.243 | 44.047 | 62.666 | 1.00 | 46.39 | A | C |
| ATOM | 1813 | O | TYR | A | 246 | 46.620 | 43.151 | 62.110 | 1.00 | 45.89 | A | O |
| ATOM | 1814 | N | HIS | A | 247 | 47.497 | 44.041 | 63.969 | 1.00 | 48.04 | A | N |
| ATOM | 1815 | CA | HIS | A | 247 | 47.029 | 42.955 | 64.821 | 1.00 | 50.73 | A | C |
| ATOM | 1816 | CB | HIS | A | 247 | 47.418 | 43.212 | 66.285 | 1.00 | 57.15 | A | C |
| ATOM | 1817 | CG | HIS | A | 247 | 47.059 | 42.090 | 67.216 | 1.00 | 65.14 | A | C |
| ATOM | 1818 | CD2 | HIS | A | 247 | 45.913 | 41.810 | 67.874 | 1.00 | 67.92 | A | C |
| ATOM | 1819 | ND1 | HIS | A | 247 | 47.937 | 41.068 | 67.521 | 1.00 | 70.91 | A | N |
| ATOM | 1820 | CE1 | HIS | A | 247 | 47.340 | 40.208 | 68.328 | 1.00 | 69.69 | A | C |
| ATOM | 1821 | NE2 | HIS | A | 247 | 46.112 | 40.630 | 68.560 | 1.00 | 67.02 | A | N |
| ATOM | 1822 | C | HIS | A | 247 | 45.510 | 42.815 | 64.713 | 1.00 | 48.21 | A | C |
| ATOM | 1823 | O | HIS | A | 247 | 45.001 | 41.728 | 64.439 | 1.00 | 46.41 | A | O |
| ATOM | 1824 | N | MET | A | 248 | 44.790 | 43.914 | 64.924 | 1.00 | 45.66 | A | N |
| ATOM | 1825 | CA | MET | A | 248 | 43.329 | 43.869 | 64.864 | 1.00 | 42.22 | A | C |
| ATOM | 1826 | CB | MET | A | 248 | 42.732 | 45.246 | 65.154 | 1.00 | 36.87 | A | C |
| ATOM | 1827 | CG | MET | A | 248 | 43.096 | 45.775 | 66.520 | 1.00 | 37.76 | A | C |
| ATOM | 1828 | SD | MET | A | 248 | 42.373 | 47.390 | 66.847 | 1.00 | 45.63 | A | S |
| ATOM | 1829 | CE | MET | A | 248 | 43.473 | 48.458 | 66.027 | 1.00 | 32.36 | A | C |
| ATOM | 1830 | C | MET | A | 248 | 42.844 | 43.375 | 63.513 | 1.00 | 37.88 | A | C |
| ATOM | 1831 | O | MET | A | 248 | 42.047 | 42.447 | 63.447 | 1.00 | 36.98 | A | O |
| ATOM | 1832 | N | LEU | A | 249 | 43.330 | 43.994 | 62.443 | 1.00 | 40.04 | A | N |
| ATOM | 1833 | CA | LEU | A | 249 | 42.936 | 43.621 | 61.082 | 1.00 | 40.03 | A | C |
| ATOM | 1834 | CB | LEU | A | 249 | 43.591 | 44.572 | 60.076 | 1.00 | 36.91 | A | C |
| ATOM | 1835 | CG | LEU | A | 249 | 42.983 | 45.980 | 59.994 | 1.00 | 36.38 | A | C |
| ATOM | 1836 | CD1 | LEU | A | 249 | 43.842 | 46.869 | 59.102 | 1.00 | 35.85 | A | C |
| ATOM | 1837 | CD2 | LEU | A | 249 | 41.558 | 45.889 | 59.423 | 1.00 | 33.93 | A | C |
| ATOM | 1838 | C | LEU | A | 249 | 43.259 | 42.172 | 60.720 | 1.00 | 40.31 | A | C |
| ATOM | 1839 | O | LEU | A | 249 | 42.443 | 41.477 | 60.112 | 1.00 | 41.79 | A | O |
| ATOM | 1840 | N | PHE | A | 250 | 44.444 | 41.709 | 61.098 | 1.00 | 42.83 | A | N |
| ATOM | 1841 | CA | PHE | A | 250 | 44.847 | 40.340 | 60.803 | 1.00 | 42.94 | A | C |
| ATOM | 1842 | CB | PHE | A | 250 | 46.282 | 40.089 | 61.282 | 1.00 | 45.13 | A | C |
| ATOM | 1843 | CG | PHE | A | 250 | 46.775 | 38.695 | 61.011 | 1.00 | 45.35 | A | C |
| ATOM | 1844 | CD1 | PHE | A | 250 | 46.909 | 38.233 | 59.702 | 1.00 | 45.66 | A | C |
| ATOM | 1845 | CD2 | PHE | A | 250 | 47.092 | 37.835 | 62.064 | 1.00 | 48.91 | A | C |
| ATOM | 1846 | CE1 | PHE | A | 250 | 47.351 | 36.936 | 59.439 | 1.00 | 45.62 | A | C |
| ATOM | 1847 | CE2 | PHE | A | 250 | 47.540 | 36.524 | 61.810 | 1.00 | 48.16 | A | C |
| ATOM | 1848 | CZ | PHE | A | 250 | 47.668 | 36.080 | 60.491 | 1.00 | 46.18 | A | C |
| ATOM | 1849 | C | PHE | A | 250 | 43.901 | 39.352 | 61.477 | 1.00 | 41.33 | A | C |
| ATOM | 1850 | O | PHE | A | 250 | 43.496 | 38.342 | 60.881 | 1.00 | 41.88 | A | O |
| ATOM | 1851 | N | GLU | A | 251 | 43.553 | 39.636 | 62.726 | 1.00 | 41.55 | A | N |
| ATOM | 1852 | CA | GLU | A | 251 | 42.653 | 38.762 | 63.461 | 1.00 | 41.49 | A | C |
| ATOM | 1853 | CB | GLU | A | 251 | 42.447 | 39.265 | 64.876 | 1.00 | 45.54 | A | C |
| ATOM | 1854 | CG | GLU | A | 251 | 41.665 | 38.284 | 65.717 | 1.00 | 59.11 | A | C |
| ATOM | 1855 | CD | GLU | A | 251 | 42.264 | 36.879 | 65.650 | 1.00 | 65.40 | A | C |
| ATOM | 1856 | OE1 | GLU | A | 251 | 43.493 | 36.739 | 65.879 | 1.00 | 62.44 | A | O |
| ATOM | 1857 | OE2 | GLU | A | 251 | 41.505 | 35.921 | 65.366 | 1.00 | 67.91 | A | O |
| ATOM | 1858 | C | GLU | A | 251 | 41.299 | 38.669 | 62.775 | 1.00 | 41.60 | A | C |
| ATOM | 1859 | O | GLU | A | 251 | 40.723 | 37.585 | 62.668 | 1.00 | 41.21 | A | O |
| ATOM | 1860 | N | LEU | A | 252 | 40.799 | 39.807 | 62.298 | 1.00 | 37.86 | A | N |
| ATOM | 1861 | CA | LEU | A | 252 | 39.498 | 39.848 | 61.646 | 1.00 | 38.04 | A | C |
| ATOM | 1862 | CB | LEU | A | 252 | 39.002 | 41.299 | 61.526 | 1.00 | 33.60 | A | C |
| ATOM | 1863 | CG | LEU | A | 252 | 38.716 | 42.000 | 62.858 | 1.00 | 34.61 | A | C |
| ATOM | 1864 | CD1 | LEU | A | 252 | 38.375 | 43.481 | 62.620 | 1.00 | 30.96 | A | C |
| ATOM | 1865 | CD2 | LEU | A | 252 | 37.569 | 41.289 | 63.566 | 1.00 | 29.33 | A | C |
| ATOM | 1866 | C | LEU | A | 252 | 39.538 | 39.192 | 60.279 | 1.00 | 37.19 | A | C |
| ATOM | 1867 | O | LEU | A | 252 | 38.606 | 38.491 | 59.909 | 1.00 | 37.53 | A | O |
| ATOM | 1868 | N | PHE | A | 253 | 40.608 | 39.416 | 59.523 | 1.00 | 36.43 | A | N |
| ATOM | 1869 | CA | PHE | A | 253 | 40.707 | 38.801 | 58.209 | 1.00 | 38.16 | A | C |
| ATOM | 1870 | CB | PHE | A | 253 | 41.960 | 39.269 | 57.482 | 1.00 | 37.47 | A | C |
| ATOM | 1871 | CG | PHE | A | 253 | 41.780 | 40.560 | 56.731 | 1.00 | 33.62 | A | C |
| ATOM | 1872 | CD1 | PHE | A | 253 | 40.818 | 40.670 | 55.731 | 1.00 | 30.18 | A | C |
| ATOM | 1873 | CD2 | PHE | A | 253 | 42.602 | 41.645 | 56.989 | 1.00 | 32.02 | A | C |
| ATOM | 1874 | CE1 | PHE | A | 253 | 40.687 | 41.847 | 54.995 | 1.00 | 32.94 | A | C |
| ATOM | 1875 | CE2 | PHE | A | 253 | 42.482 | 42.830 | 56.261 | 1.00 | 27.25 | A | C |
| ATOM | 1876 | CZ | PHE | A | 253 | 41.526 | 42.931 | 55.264 | 1.00 | 32.00 | A | C |
| ATOM | 1877 | C | PHE | A | 253 | 40.715 | 37.286 | 58.338 | 1.00 | 40.82 | A | C |
| ATOM | 1878 | O | PHE | A | 253 | 40.065 | 36.583 | 57.560 | 1.00 | 43.06 | A | O |
| ATOM | 1879 | N | LYS | A | 254 | 41.431 | 36.773 | 59.327 | 1.00 | 42.26 | A | N |
| ATOM | 1880 | CA | LYS | A | 254 | 41.461 | 35.330 | 59.526 | 1.00 | 43.38 | A | C |
| ATOM | 1881 | CB | LYS | A | 254 | 42.354 | 34.971 | 60.702 | 1.00 | 45.09 | A | C |
| ATOM | 1882 | CG | LYS | A | 254 | 43.788 | 34.729 | 60.291 | 1.00 | 53.86 | A | C |
| ATOM | 1883 | CD | LYS | A | 254 | 44.762 | 35.047 | 61.416 | 1.00 | 61.32 | A | C |
| ATOM | 1884 | CE | LYS | A | 254 | 44.494 | 34.258 | 62.684 | 1.00 | 62.84 | A | C |
| ATOM | 1885 | NZ | LYS | A | 254 | 45.534 | 34.566 | 63.704 | 1.00 | 65.53 | A | N |
| ATOM | 1886 | C | LYS | A | 254 | 40.069 | 34.756 | 59.746 | 1.00 | 43.15 | A | C |
| ATOM | 1887 | O | LYS | A | 254 | 39.681 | 33.787 | 59.083 | 1.00 | 44.07 | A | O |
| ATOM | 1888 | N | ASN | A | 255 | 39.315 | 35.348 | 60.671 | 1.00 | 41.30 | A | N |
| ATOM | 1889 | CA | ASN | A | 255 | 37.962 | 34.870 | 60.954 | 1.00 | 36.16 | A | C |
| ATOM | 1890 | CB | ASN | A | 255 | 37.328 | 35.653 | 62.100 | 1.00 | 38.55 | A | C |
| ATOM | 1891 | CG | ASN | A | 255 | 38.031 | 35.420 | 63.428 | 1.00 | 42.41 | A | C |
| ATOM | 1892 | OD1 | ASN | A | 255 | 38.474 | 34.311 | 63.713 | 1.00 | 43.76 | A | O |
| ATOM | 1893 | ND2 | ASN | A | 255 | 38.118 | 36.466 | 64.253 | 1.00 | 35.83 | A | N |
| ATOM | 1894 | C | ASN | A | 255 | 37.067 | 34.997 | 59.730 | 1.00 | 36.31 | A | C |
| ATOM | 1895 | O | ASN | A | 255 | 36.321 | 34.075 | 59.400 | 1.00 | 38.24 | A | O |
| ATOM | 1896 | N | ALA | A | 256 | 37.129 | 36.142 | 59.062 | 1.00 | 30.92 | A | N |
| ATOM | 1897 | CA | ALA | A | 256 | 36.304 | 36.365 | 57.891 | 1.00 | 33.89 | A | C |
| ATOM | 1898 | CB | ALA | A | 256 | 36.443 | 37.815 | 57.425 | 1.00 | 28.62 | A | C |
| ATOM | 1899 | C | ALA | A | 256 | 36.691 | 35.402 | 56.761 | 1.00 | 38.06 | A | C |
| ATOM | 1900 | O | ALA | A | 256 | 35.851 | 35.023 | 55.946 | 1.00 | 38.54 | A | O |
| ATOM | 1901 | N | MET | A | 257 | 37.962 | 35.019 | 56.700 | 1.00 | 38.18 | A | N |
| ATOM | 1902 | CA | MET | A | 257 | 38.403 | 34.092 | 55.662 | 1.00 | 41.50 | A | C |
| ATOM | 1903 | CB | MET | A | 257 | 39.919 | 34.138 | 55.508 | 1.00 | 42.90 | A | C |
| ATOM | 1904 | CG | MET | A | 257 | 40.388 | 35.382 | 54.787 | 1.00 | 48.13 | A | C |
| ATOM | 1905 | SD | MET | A | 257 | 42.148 | 35.383 | 54.496 | 1.00 | 51.04 | A | S |
| ATOM | 1906 | CE | MET | A | 257 | 42.297 | 36.884 | 53.429 | 1.00 | 38.17 | A | C |
| ATOM | 1907 | C | MET | A | 257 | 37.970 | 32.678 | 55.990 | 1.00 | 40.54 | A | C |
| ATOM | 1908 | O | MET | A | 257 | 37.429 | 31.984 | 55.148 | 1.00 | 39.61 | A | O |
| ATOM | 1909 | N | ARG | A | 258 | 38.200 | 32.257 | 57.225 | 1.00 | 43.13 | A | N |
| ATOM | 1910 | CA | ARG | A | 258 | 37.818 | 30.919 | 57.646 | 1.00 | 44.85 | A | C |
| ATOM | 1911 | CB | ARG | A | 258 | 38.139 | 30.750 | 59.120 | 1.00 | 49.32 | A | C |
| ATOM | 1912 | CG | ARG | A | 258 | 37.529 | 29.541 | 59.781 | 1.00 | 62.78 | A | C |
| ATOM | 1913 | CD | ARG | A | 258 | 38.083 | 29.437 | 61.193 | 1.00 | 73.00 | A | C |
| ATOM | 1914 | NE | ARG | A | 258 | 37.159 | 28.819 | 62.136 | 1.00 | 84.59 | A | N |
| ATOM | 1915 | CZ | ARG | A | 258 | 36.829 | 27.531 | 62.136 | 1.00 | 90.25 | A | C |
| ATOM | 1916 | NH1 | ARG | A | 258 | 37.353 | 26.708 | 61.236 | 1.00 | 92.60 | A | N |
| ATOM | 1917 | NH2 | ARG | A | 258 | 35.975 | 27.066 | 63.041 | 1.00 | 92.77 | A | N |
| ATOM | 1918 | C | ARG | A | 258 | 36.331 | 30.684 | 57.393 | 1.00 | 46.53 | A | C |
| ATOM | 1919 | O | ARG | A | 258 | 35.942 | 29.646 | 56.855 | 1.00 | 44.38 | A | O |
| ATOM | 1920 | N | ALA | A | 259 | 35.506 | 31.661 | 57.760 | 1.00 | 42.76 | A | N |
| ATOM | 1921 | CA | ALA | A | 259 | 34.061 | 31.544 | 57.585 | 1.00 | 42.47 | A | C |
| ATOM | 1922 | CB | ALA | A | 259 | 33.331 | 32.600 | 58.443 | 1.00 | 35.92 | A | C |
| ATOM | 1923 | C | ALA | A | 259 | 33.577 | 31.625 | 56.138 | 1.00 | 40.87 | A | C |
| ATOM | 1924 | O | ALA | A | 259 | 32.620 | 30.943 | 55.785 | 1.00 | 44.44 | A | O |
| ATOM | 1925 | N | THR | A | 260 | 34.200 | 32.464 | 55.308 | 1.00 | 40.97 | A | N |
| ATOM | 1926 | CA | THR | A | 260 | 33.775 | 32.578 | 53.910 | 1.00 | 43.70 | A | C |
| ATOM | 1927 | CB | THR | A | 260 | 34.498 | 33.717 | 53.166 | 1.00 | 44.30 | A | C |
| ATOM | 1928 | OG1 | THR | A | 260 | 34.265 | 34.964 | 53.830 | 1.00 | 43.02 | A | O |
| ATOM | 1929 | CG2 | THR | A | 260 | 33.969 | 33.826 | 51.737 | 1.00 | 43.95 | A | C |
| ATOM | 1930 | C | THR | A | 260 | 34.078 | 31.279 | 53.158 | 1.00 | 46.65 | A | C |
| ATOM | 1931 | O | THR | A | 260 | 33.278 | 30.812 | 52.345 | 1.00 | 45.26 | A | O |
| ATOM | 1932 | N | VAL | A | 261 | 35.247 | 30.715 | 53.446 | 1.00 | 48.37 | A | N |
| ATOM | 1933 | CA | VAL | A | 261 | 35.707 | 29.480 | 52.833 | 1.00 | 49.41 | A | C |
| ATOM | 1934 | CB | VAL | A | 261 | 37.198 | 29.203 | 53.209 | 1.00 | 50.34 | A | C |
| ATOM | 1935 | CG1 | VAL | A | 261 | 37.568 | 27.755 | 52.902 | 1.00 | 51.57 | A | C |
| ATOM | 1936 | CG2 | VAL | A | 261 | 38.117 | 30.152 | 52.437 | 1.00 | 48.30 | A | C |
| ATOM | 1937 | C | VAL | A | 261 | 34.852 | 28.303 | 53.286 | 1.00 | 50.79 | A | C |
| ATOM | 1938 | O | VAL | A | 261 | 34.299 | 27.568 | 52.466 | 1.00 | 52.47 | A | O |
| ATOM | 1939 | N | GLU | A | 262 | 34.734 | 28.135 | 54.594 | 1.00 | 50.57 | A | N |
| ATOM | 1940 | CA | GLU | A | 262 | 33.974 | 27.028 | 55.141 | 1.00 | 52.80 | A | C |
| ATOM | 1941 | CB | GLU | A | 262 | 34.189 | 26.952 | 56.651 | 1.00 | 55.23 | A | C |
| ATOM | 1942 | CG | GLU | A | 262 | 35.633 | 26.706 | 57.042 | 1.00 | 58.04 | A | C |
| ATOM | 1943 | CD | GLU | A | 262 | 35.777 | 26.285 | 58.484 | 1.00 | 63.11 | A | C |
| ATOM | 1944 | OE1 | GLU | A | 262 | 36.913 | 25.972 | 58.898 | 1.00 | 66.77 | A | O |
| ATOM | 1945 | OE2 | GLU | A | 262 | 34.756 | 26.267 | 59.205 | 1.00 | 65.64 | A | O |
| ATOM | 1946 | C | GLU | A | 262 | 32.483 | 27.037 | 54.830 | 1.00 | 54.78 | A | C |
| ATOM | 1947 | O | GLU | A | 262 | 31.881 | 25.975 | 54.666 | 1.00 | 56.27 | A | O |
| ATOM | 1948 | N | SER | A | 263 | 31.883 | 28.222 | 54.745 | 1.00 | 54.59 | A | N |
| ATOM | 1949 | CA | SER | A | 263 | 30.455 | 28.317 | 54.456 | 1.00 | 53.40 | A | C |
| ATOM | 1950 | CB | SER | A | 263 | 29.897 | 29.667 | 54.922 | 1.00 | 53.73 | A | C |
| ATOM | 1951 | OG | SER | A | 263 | 30.405 | 30.741 | 54.144 | 1.00 | 56.59 | A | O |
| ATOM | 1952 | C | SER | A | 263 | 30.165 | 28.135 | 52.967 | 1.00 | 54.65 | A | C |
| ATOM | 1953 | O | SER | A | 263 | 29.015 | 27.949 | 52.575 | 1.00 | 55.17 | A | O |
| ATOM | 1954 | N | HIS | A | 264 | 31.202 | 28.201 | 52.137 | 1.00 | 54.81 | A | N |
| ATOM | 1955 | CA | HIS | A | 264 | 31.024 | 28.028 | 50.699 | 1.00 | 55.70 | A | C |
| ATOM | 1956 | CB | HIS | A | 264 | 31.452 | 29.279 | 49.939 | 1.00 | 51.70 | A | C |
| ATOM | 1957 | CG | HIS | A | 264 | 30.571 | 30.461 | 50.179 | 1.00 | 52.63 | A | C |
| ATOM | 1958 | CD2 | HIS | A | 264 | 29.684 | 31.090 | 49.374 | 1.00 | 51.08 | A | C |
| ATOM | 1959 | ND1 | HIS | A | 264 | 30.548 | 31.137 | 51.381 | 1.00 | 48.93 | A | N |
| ATOM | 1960 | CE1 | HIS | A | 264 | 29.685 | 32.133 | 51.303 | 1.00 | 47.89 | A | C |
| ATOM | 1961 | NE2 | HIS | A | 264 | 29.146 | 32.128 | 50.098 | 1.00 | 50.78 | A | N |
| ATOM | 1962 | C | HIS | A | 264 | 31.792 | 26.843 | 50.141 | 1.00 | 57.88 | A | C |
| ATOM | 1963 | O | HIS | A | 264 | 32.249 | 26.886 | 49.004 | 1.00 | 57.14 | A | O |
| ATOM | 1964 | N | GLU | A | 265 | 31.941 | 25.786 | 50.926 | 1.00 | 62.19 | A | N |
| ATOM | 1965 | CA | GLU | A | 265 | 32.662 | 24.622 | 50.442 | 1.00 | 68.50 | A | C |
| ATOM | 1966 | CB | GLU | A | 265 | 32.987 | 23.672 | 51.589 | 1.00 | 71.52 | A | C |
| ATOM | 1967 | CG | GLU | A | 265 | 33.960 | 24.258 | 52.584 | 1.00 | 80.08 | A | C |
| ATOM | 1968 | CD | GLU | A | 265 | 34.473 | 23.227 | 53.558 | 1.00 | 83.57 | A | C |
| ATOM | 1969 | OE1 | GLU | A | 265 | 33.639 | 22.544 | 54.187 | 1.00 | 85.88 | A | O |
| ATOM | 1970 | OE2 | GLU | A | 265 | 35.707 | 23.100 | 53.694 | 1.00 | 86.87 | A | O |
| ATOM | 1971 | C | GLU | A | 265 | 31.832 | 23.906 | 49.392 | 1.00 | 71.33 | A | C |
| ATOM | 1972 | O | GLU | A | 265 | 31.950 | 22.694 | 49.217 | 1.00 | 71.63 | A | O |
| ATOM | 1973 | N | SER | A | 266 | 30.995 | 24.674 | 48.695 | 1.00 | 72.71 | A | N |
| ATOM | 1974 | CA | SER | A | 266 | 30.130 | 24.146 | 47.653 | 1.00 | 73.19 | A | C |
| ATOM | 1975 | CB | SER | A | 266 | 28.953 | 23.389 | 48.272 | 1.00 | 70.78 | A | C |
| ATOM | 1976 | OG | SER | A | 266 | 29.396 | 22.368 | 49.147 | 1.00 | 71.51 | A | O |
| ATOM | 1977 | C | SER | A | 266 | 29.585 | 25.254 | 46.758 | 1.00 | 76.31 | A | C |
| ATOM | 1978 | O | SER | A | 266 | 28.377 | 25.468 | 46.716 | 1.00 | 77.40 | A | O |
| ATOM | 1979 | N | SER | A | 267 | 30.462 | 25.964 | 46.048 | 1.00 | 79.29 | A | N |
| ATOM | 1980 | CA | SER | A | 267 | 30.012 | 27.023 | 45.144 | 1.00 | 83.76 | A | C |
| ATOM | 1981 | CB | SER | A | 267 | 29.056 | 27.976 | 45.875 | 1.00 | 82.83 | A | C |
| ATOM | 1982 | OG | SER | A | 267 | 29.400 | 28.113 | 47.239 | 1.00 | 80.59 | A | O |
| ATOM | 1983 | C | SER | A | 267 | 31.124 | 27.817 | 44.454 | 1.00 | 87.35 | A | C |
| ATOM | 1984 | O | SER | A | 267 | 31.849 | 27.281 | 43.617 | 1.00 | 88.98 | A | O |
| ATOM | 1985 | N | LEU | A | 268 | 31.243 | 29.101 | 44.780 | 1.00 | 91.61 | A | N |
| ATOM | 1986 | CA | LEU | A | 268 | 32.272 | 29.934 | 44.173 | 1.00 | 92.99 | A | C |
| ATOM | 1987 | CB | LEU | A | 268 | 31.624 | 31.046 | 43.338 | 1.00 | 97.69 | A | C |
| ATOM | 1988 | CG | LEU | A | 268 | 30.857 | 32.203 | 43.978 | 1.00 | 100.98 | A | C |
| ATOM | 1989 | CD1 | LEU | A | 268 | 30.225 | 33.024 | 42.868 | 1.00 | 103.39 | A | C |
| ATOM | 1990 | CD2 | LEU | A | 268 | 29.797 | 31.695 | 44.936 | 1.00 | 102.92 | A | C |
| ATOM | 1991 | C | LEU | A | 268 | 33.218 | 30.505 | 45.238 | 1.00 | 91.03 | A | C |
| ATOM | 1992 | O | LEU | A | 268 | 34.268 | 29.906 | 45.484 | 1.00 | 93.15 | A | O |
| ATOM | 1993 | N | ILE | A | 269 | 32.852 | 31.642 | 45.852 | 1.00 | 83.46 | A | N |
| ATOM | 1994 | CA | ILE | A | 269 | 33.640 | 32.303 | 46.917 | 1.00 | 74.36 | A | C |
| ATOM | 1995 | CB | ILE | A | 269 | 35.111 | 31.799 | 46.943 | 1.00 | 74.47 | A | C |
| ATOM | 1996 | CG2 | ILE | A | 269 | 35.863 | 32.259 | 45.694 | 1.00 | 78.33 | A | C |
| ATOM | 1997 | CG1 | ILE | A | 269 | 35.808 | 32.291 | 48.205 | 1.00 | 74.19 | A | C |
| ATOM | 1998 | CD1 | ILE | A | 269 | 37.191 | 31.719 | 48.390 | 1.00 | 70.97 | A | C |
| ATOM | 1999 | C | ILE | A | 269 | 33.646 | 33.851 | 46.891 | 1.00 | 66.30 | A | C |
| ATOM | 2000 | O | ILE | A | 269 | 34.492 | 34.478 | 46.252 | 1.00 | 65.06 | A | O |
| ATOM | 2001 | N | PRO | A | 271 | 32.708 | 34.486 | 47.616 | 1.00 | 59.45 | A | N |
| ATOM | 2002 | CD | PRO | A | 271 | 31.713 | 33.880 | 48.517 | 1.00 | 55.69 | A | C |
| ATOM | 2003 | CA | PRO | A | 271 | 32.617 | 35.952 | 47.661 | 1.00 | 54.20 | A | C |
| ATOM | 2004 | CB | PRO | A | 271 | 31.317 | 36.190 | 48.421 | 1.00 | 53.23 | A | C |
| ATOM | 2005 | CG | PRO | A | 271 | 31.298 | 35.052 | 49.371 | 1.00 | 55.22 | A | C |
| ATOM | 2006 | C | PRO | A | 271 | 33.810 | 36.638 | 48.330 | 1.00 | 49.09 | A | C |
| ATOM | 2007 | O | PRO | A | 271 | 34.460 | 36.073 | 49.218 | 1.00 | 46.62 | A | O |
| ATOM | 2008 | N | PRO | A | 272 | 34.104 | 37.874 | 47.911 | 1.00 | 45.20 | A | N |
| ATOM | 2009 | CD | PRO | A | 272 | 33.443 | 38.629 | 46.831 | 1.00 | 41.66 | A | C |
| ATOM | 2010 | CA | PRO | A | 272 | 35.223 | 38.633 | 48.473 | 1.00 | 45.22 | A | C |
| ATOM | 2011 | CB | PRO | A | 272 | 35.499 | 39.677 | 47.399 | 1.00 | 43.87 | A | C |
| ATOM | 2012 | CG | PRO | A | 272 | 34.111 | 39.998 | 46.919 | 1.00 | 42.79 | A | C |
| ATOM | 2013 | C | PRO | A | 272 | 34.929 | 39.288 | 49.817 | 1.00 | 45.34 | A | C |
| ATOM | 2014 | O | PRO | A | 272 | 33.772 | 39.455 | 50.219 | 1.00 | 44.11 | A | O |
| ATOM | 2015 | N | ILE | A | 273 | 36.004 | 39.657 | 50.502 | 1.00 | 45.58 | A | N |
| ATOM | 2016 | CA | ILE | A | 273 | 35.911 | 40.343 | 51.770 | 1.00 | 41.46 | A | C |
| ATOM | 2017 | CB | ILE | A | 273 | 37.044 | 39.934 | 52.681 | 1.00 | 37.41 | A | C |
| ATOM | 2018 | CG2 | ILE | A | 273 | 37.068 | 40.822 | 53.907 | 1.00 | 39.62 | A | C |
| ATOM | 2019 | CG1 | ILE | A | 273 | 36.857 | 38.474 | 53.067 | 1.00 | 37.34 | A | C |
| ATOM | 2020 | CD1 | ILE | A | 273 | 37.945 | 37.918 | 53.929 | 1.00 | 40.04 | A | C |
| ATOM | 2021 | C | ILE | A | 273 | 36.034 | 41.810 | 51.415 | 1.00 | 42.68 | A | C |
| ATOM | 2022 | O | ILE | A | 273 | 36.994 | 42.208 | 50.759 | 1.00 | 46.14 | A | O |
| ATOM | 2023 | N | LYS | A | 274 | 35.051 | 42.610 | 51.813 | 1.00 | 41.56 | A | N |
| ATOM | 2024 | CA | LYS | A | 274 | 35.076 | 44.034 | 51.499 | 1.00 | 41.88 | A | C |
| ATOM | 2025 | CB | LYS | A | 274 | 33.728 | 44.475 | 50.940 | 1.00 | 43.31 | A | C |
| ATOM | 2026 | CG | LYS | A | 274 | 33.264 | 43.569 | 49.834 | 1.00 | 50.97 | A | C |
| ATOM | 2027 | CD | LYS | A | 274 | 32.070 | 44.110 | 49.086 | 1.00 | 56.97 | A | C |
| ATOM | 2028 | CE | LYS | A | 274 | 31.674 | 43.116 | 48.003 | 1.00 | 60.48 | A | C |
| ATOM | 2029 | NZ | LYS | A | 274 | 30.869 | 43.745 | 46.928 | 1.00 | 67.90 | A | N |
| ATOM | 2030 | C | LYS | A | 274 | 35.420 | 44.874 | 52.709 | 1.00 | 41.23 | A | C |
| ATOM | 2031 | O | LYS | A | 274 | 34.922 | 44.648 | 53.805 | 1.00 | 45.19 | A | O |
| ATOM | 2032 | N | VAL | A | 275 | 36.286 | 45.846 | 52.503 | 1.00 | 37.02 | A | N |
| ATOM | 2033 | CA | VAL | A | 275 | 36.686 | 46.703 | 53.573 | 1.00 | 36.75 | A | C |
| ATOM | 2034 | CB | VAL | A | 275 | 38.191 | 46.537 | 53.916 | 1.00 | 34.64 | A | C |
| ATOM | 2035 | CG1 | VAL | A | 275 | 38.576 | 47.515 | 55.013 | 1.00 | 34.72 | A | C |
| ATOM | 2036 | CG2 | VAL | A | 275 | 38.471 | 45.128 | 54.380 | 1.00 | 36.08 | A | C |
| ATOM | 2037 | C | VAL | A | 275 | 36.462 | 48.137 | 53.184 | 1.00 | 38.67 | A | C |
| ATOM | 2038 | O | VAL | A | 275 | 36.773 | 48.543 | 52.064 | 1.00 | 40.91 | A | O |
| ATOM | 2039 | N | MET | A | 276 | 35.908 | 48.907 | 54.111 | 1.00 | 40.28 | A | N |
| ATOM | 2040 | CA | MET | A | 276 | 35.726 | 50.317 | 53.870 | 1.00 | 40.94 | A | C |
| ATOM | 2041 | CB | MET | A | 276 | 34.265 | 50.730 | 53.889 | 1.00 | 46.92 | A | C |
| ATOM | 2042 | CG | MET | A | 276 | 34.119 | 52.179 | 53.455 | 1.00 | 58.99 | A | C |
| ATOM | 2043 | SD | MET | A | 276 | 32.453 | 52.810 | 53.474 | 1.00 | 69.65 | A | S |
| ATOM | 2044 | CE | MET | A | 276 | 32.596 | 54.001 | 54.764 | 1.00 | 73.42 | A | C |
| ATOM | 2045 | C | MET | A | 276 | 36.451 | 51.094 | 54.954 | 1.00 | 39.88 | A | C |
| ATOM | 2046 | O | MET | A | 276 | 36.335 | 50.794 | 56.151 | 1.00 | 39.25 | A | O |
| ATOM | 2047 | N | VAL | A | 277 | 37.220 | 52.082 | 54.523 | 1.00 | 36.23 | A | N |
| ATOM | 2048 | CA | VAL | A | 277 | 37.922 | 52.934 | 55.442 | 1.00 | 36.00 | A | C |
| ATOM | 2049 | CB | VAL | A | 277 | 39.424 | 53.017 | 55.118 | 1.00 | 33.76 | A | C |
| ATOM | 2050 | CG1 | VAL | A | 277 | 40.099 | 53.931 | 56.121 | 1.00 | 30.31 | A | C |
| ATOM | 2051 | CG2 | VAL | A | 277 | 40.053 | 51.635 | 55.161 | 1.00 | 30.07 | A | C |
| ATOM | 2052 | C | VAL | A | 277 | 37.282 | 54.295 | 55.262 | 1.00 | 36.61 | A | C |
| ATOM | 2053 | O | VAL | A | 277 | 37.342 | 54.875 | 54.165 | 1.00 | 40.28 | A | O |
| ATOM | 2054 | N | ALA | A | 278 | 36.661 | 54.790 | 56.333 | 1.00 | 35.60 | A | N |
| ATOM | 2055 | CA | ALA | A | 278 | 35.986 | 56.083 | 56.325 | 1.00 | 33.91 | A | C |
| ATOM | 2056 | CB | ALA | A | 278 | 34.499 | 55.904 | 56.626 | 1.00 | 27.01 | A | C |
| ATOM | 2057 | C | ALA | A | 278 | 36.609 | 57.015 | 57.350 | 1.00 | 33.52 | A | C |
| ATOM | 2058 | O | ALA | A | 278 | 36.832 | 56.632 | 58.486 | 1.00 | 37.97 | A | O |
| ATOM | 2059 | N | LEU | A | 279 | 36.883 | 58.243 | 56.940 | 1.00 | 34.96 | A | N |
| ATOM | 2060 | CA | LEU | A | 279 | 37.470 | 59.239 | 57.810 | 1.00 | 36.36 | A | C |
| ATOM | 2061 | CB | LEU | A | 279 | 38.809 | 59.713 | 57.235 | 1.00 | 37.45 | A | C |
| ATOM | 2062 | CG | LEU | A | 279 | 39.386 | 60.983 | 57.859 | 1.00 | 39.10 | A | C |
| ATOM | 2063 | CD1 | LEU | A | 279 | 39.576 | 60.759 | 59.343 | 1.00 | 39.48 | A | C |
| ATOM | 2064 | CD2 | LEU | A | 279 | 40.711 | 61.356 | 57.185 | 1.00 | 39.22 | A | C |
| ATOM | 2065 | C | LEU | A | 279 | 36.526 | 60.419 | 57.972 | 1.00 | 40.17 | A | C |
| ATOM | 2066 | O | LEU | A | 279 | 36.239 | 61.130 | 57.002 | 1.00 | 43.00 | A | O |
| ATOM | 2067 | N | GLY | A | 280 | 36.042 | 60.611 | 59.200 | 1.00 | 40.85 | A | N |
| ATOM | 2068 | CA | GLY | A | 280 | 35.140 | 61.706 | 59.513 | 1.00 | 45.08 | A | C |
| ATOM | 2069 | C | GLY | A | 280 | 35.848 | 62.762 | 60.348 | 1.00 | 48.74 | A | C |
| ATOM | 2070 | O | GLY | A | 280 | 37.060 | 62.684 | 60.524 | 1.00 | 47.56 | A | O |
| ATOM | 2071 | N | GLU | A | 281 | 35.109 | 63.739 | 60.873 | 1.00 | 54.14 | A | N |
| ATOM | 2072 | CA | GLU | A | 281 | 35.720 | 64.810 | 61.669 | 1.00 | 60.36 | A | C |
| ATOM | 2073 | CB | GLU | A | 281 | 34.666 | 65.807 | 62.161 | 1.00 | 74.08 | A | C |
| ATOM | 2074 | CG | GLU | A | 281 | 33.844 | 66.474 | 61.069 | 1.00 | 96.21 | A | C |
| ATOM | 2075 | CD | GLU | A | 281 | 33.023 | 67.642 | 61.593 | 1.00 | 107.54 | A | C |
| ATOM | 2076 | OE1 | GLU | A | 281 | 32.586 | 67.582 | 62.763 | 1.00 | 114.01 | A | O |
| ATOM | 2077 | OE2 | GLU | A | 281 | 32.803 | 68.611 | 60.834 | 1.00 | 115.31 | A | O |
| ATOM | 2078 | C | GLU | A | 281 | 36.512 | 64.304 | 62.873 | 1.00 | 56.38 | A | C |
| ATOM | 2079 | O | GLU | A | 281 | 37.611 | 64.778 | 63.135 | 1.00 | 56.01 | A | O |
| ATOM | 2080 | N | GLU | A | 282 | 35.948 | 63.363 | 63.622 | 1.00 | 52.09 | A | N |
| ATOM | 2081 | CA | GLU | A | 282 | 36.649 | 62.826 | 64.776 | 1.00 | 48.96 | A | C |
| ATOM | 2082 | CB | GLU | A | 282 | 35.913 | 63.179 | 66.062 | 1.00 | 49.55 | A | C |
| ATOM | 2083 | CG | GLU | A | 282 | 34.488 | 62.760 | 66.100 | 1.00 | 51.87 | A | C |
| ATOM | 2084 | CD | GLU | A | 282 | 33.893 | 62.970 | 67.469 | 1.00 | 56.14 | A | C |
| ATOM | 2085 | OE1 | GLU | A | 282 | 34.275 | 62.226 | 68.402 | 1.00 | 52.37 | A | O |
| ATOM | 2086 | OE2 | GLU | A | 282 | 33.053 | 63.885 | 67.608 | 1.00 | 55.76 | A | O |
| ATOM | 2087 | C | GLU | A | 282 | 36.898 | 61.318 | 64.732 | 1.00 | 44.93 | A | C |
| ATOM | 2088 | O | GLU | A | 282 | 37.783 | 60.827 | 65.425 | 1.00 | 43.71 | A | O |
| ATOM | 2089 | N | ASP | A | 283 | 36.133 | 60.589 | 63.921 | 1.00 | 39.48 | A | N |
| ATOM | 2090 | CA | ASP | A | 283 | 36.310 | 59.140 | 63.808 | 1.00 | 38.99 | A | C |
| ATOM | 2091 | CB | ASP | A | 283 | 34.968 | 58.399 | 63.880 | 1.00 | 38.41 | A | C |
| ATOM | 2092 | CG | ASP | A | 283 | 34.393 | 58.311 | 65.276 | 1.00 | 39.52 | A | C |
| ATOM | 2093 | OD1 | ASP | A | 283 | 35.045 | 58.738 | 66.260 | 1.00 | 46.17 | A | O |
| ATOM | 2094 | OD2 | ASP | A | 283 | 33.261 | 57.792 | 65.379 | 1.00 | 40.94 | A | O |
| ATOM | 2095 | C | ASP | A | 283 | 36.970 | 58.701 | 62.501 | 1.00 | 38.54 | A | C |
| ATOM | 2096 | O | ASP | A | 283 | 36.811 | 59.336 | 61.450 | 1.00 | 37.28 | A | O |
| ATOM | 2097 | N | LEU | A | 284 | 37.710 | 57.599 | 62.589 | 1.00 | 36.76 | A | N |
| ATOM | 2098 | CA | LEU | A | 284 | 38.344 | 56.965 | 61.440 | 1.00 | 34.65 | A | C |
| ATOM | 2099 | CB | LEU | A | 284 | 39.866 | 57.034 | 61.542 | 1.00 | 36.39 | A | C |
| ATOM | 2100 | CG | LEU | A | 284 | 40.607 | 56.747 | 60.222 | 1.00 | 43.51 | A | C |
| ATOM | 2101 | CD1 | LEU | A | 284 | 42.082 | 57.081 | 60.363 | 1.00 | 44.77 | A | C |
| ATOM | 2102 | CD2 | LEU | A | 284 | 40.443 | 55.293 | 59.831 | 1.00 | 42.18 | A | C |
| ATOM | 2103 | C | LEU | A | 284 | 37.830 | 55.530 | 61.611 | 1.00 | 35.16 | A | C |
| ATOM | 2104 | O | LEU | A | 284 | 38.220 | 54.831 | 62.551 | 1.00 | 37.93 | A | O |
| ATOM | 2105 | N | SER | A | 285 | 36.928 | 55.101 | 60.734 | 1.00 | 34.01 | A | N |
| ATOM | 2106 | CA | SER | A | 285 | 36.332 | 53.775 | 60.854 | 1.00 | 31.74 | A | C |
| ATOM | 2107 | CB | SER | A | 285 | 34.823 | 53.911 | 60.917 | 1.00 | 29.44 | A | C |
| ATOM | 2108 | OG | SER | A | 285 | 34.453 | 54.971 | 61.768 | 1.00 | 32.91 | A | O |
| ATOM | 2109 | C | SER | A | 285 | 36.686 | 52.797 | 59.748 | 1.00 | 33.15 | A | C |
| ATOM | 2110 | O | SER | A | 285 | 36.765 | 53.168 | 58.576 | 1.00 | 35.63 | A | O |
| ATOM | 2111 | N | ILE | A | 286 | 36.864 | 51.538 | 60.134 | 1.00 | 33.62 | A | N |
| ATOM | 2112 | CA | ILE | A | 286 | 37.222 | 50.470 | 59.208 | 1.00 | 33.56 | A | C |
| ATOM | 2113 | CB | ILE | A | 286 | 38.609 | 49.914 | 59.513 | 1.00 | 32.07 | A | C |
| ATOM | 2114 | CG2 | ILE | A | 286 | 39.069 | 49.024 | 58.384 | 1.00 | 30.54 | A | C |
| ATOM | 2115 | CG1 | ILE | A | 286 | 39.597 | 51.072 | 59.717 | 1.00 | 37.90 | A | C |
| ATOM | 2116 | CD1 | ILE | A | 286 | 40.981 | 50.635 | 60.110 | 1.00 | 37.15 | A | C |
| ATOM | 2117 | C | ILE | A | 286 | 36.226 | 49.338 | 59.352 | 1.00 | 34.65 | A | C |
| ATOM | 2118 | O | ILE | A | 286 | 36.125 | 48.709 | 60.409 | 1.00 | 36.47 | A | O |
| ATOM | 2119 | N | LYS | A | 287 | 35.465 | 49.093 | 58.294 | 1.00 | 34.98 | A | N |
| ATOM | 2120 | CA | LYS | A | 287 | 34.502 | 48.016 | 58.332 | 1.00 | 36.04 | A | C |
| ATOM | 2121 | CB | LYS | A | 287 | 33.110 | 48.497 | 57.925 | 1.00 | 32.46 | A | C |
| ATOM | 2122 | CG | LYS | A | 287 | 32.145 | 47.334 | 57.745 | 1.00 | 35.61 | A | C |
| ATOM | 2123 | CD | LYS | A | 287 | 30.716 | 47.696 | 58.101 | 1.00 | 39.24 | A | C |
| ATOM | 2124 | CE | LYS | A | 287 | 30.095 | 48.611 | 57.090 | 1.00 | 48.30 | A | C |
| ATOM | 2125 | NZ | LYS | A | 287 | 28.702 | 48.949 | 57.510 | 1.00 | 57.62 | A | N |
| ATOM | 2126 | C | LYS | A | 287 | 34.938 | 46.898 | 57.407 | 1.00 | 36.08 | A | C |
| ATOM | 2127 | O | LYS | A | 287 | 35.339 | 47.136 | 56.276 | 1.00 | 36.81 | A | O |
| ATOM | 2128 | N | MET | A | 288 | 34.864 | 45.676 | 57.906 | 1.00 | 34.66 | A | N |
| ATOM | 2129 | CA | MET | A | 288 | 35.223 | 44.512 | 57.131 | 1.00 | 34.48 | A | C |
| ATOM | 2130 | CB | MET | A | 288 | 36.348 | 43.750 | 57.819 | 1.00 | 35.86 | A | C |
| ATOM | 2131 | CG | MET | A | 288 | 36.820 | 42.518 | 57.050 | 1.00 | 30.48 | A | C |
| ATOM | 2132 | SD | MET | A | 288 | 38.192 | 41.821 | 57.916 | 1.00 | 45.79 | A | S |
| ATOM | 2133 | CE | MET | A | 288 | 37.397 | 40.543 | 58.690 | 1.00 | 38.47 | A | C |
| ATOM | 2134 | C | MET | A | 288 | 33.981 | 43.638 | 57.035 | 1.00 | 36.92 | A | C |
| ATOM | 2135 | O | MET | A | 288 | 33.507 | 43.094 | 58.035 | 1.00 | 40.07 | A | O |
| ATOM | 2136 | N | SER | A | 289 | 33.466 | 43.497 | 55.823 | 1.00 | 35.47 | A | N |
| ATOM | 2137 | CA | SER | A | 289 | 32.262 | 42.712 | 55.580 | 1.00 | 39.28 | A | C |
| ATOM | 2138 | CB | SER | A | 289 | 31.267 | 43.533 | 54.759 | 1.00 | 36.23 | A | C |
| ATOM | 2139 | OG | SER | A | 289 | 31.037 | 44.786 | 55.372 | 1.00 | 38.21 | A | O |
| ATOM | 2140 | C | SER | A | 289 | 32.554 | 41.435 | 54.827 | 1.00 | 39.10 | A | C |
| ATOM | 2141 | O | SER | A | 289 | 33.179 | 41.475 | 53.767 | 1.00 | 42.94 | A | O |
| ATOM | 2142 | N | ASP | A | 290 | 32.101 | 40.310 | 55.377 | 1.00 | 39.19 | A | N |
| ATOM | 2143 | CA | ASP | A | 290 | 32.269 | 39.009 | 54.735 | 1.00 | 42.27 | A | C |
| ATOM | 2144 | CB | ASP | A | 290 | 33.159 | 38.089 | 55.571 | 1.00 | 43.27 | A | C |
| ATOM | 2145 | CG | ASP | A | 290 | 32.531 | 37.704 | 56.897 | 1.00 | 46.18 | A | C |
| ATOM | 2146 | OD1 | ASP | A | 290 | 31.475 | 37.034 | 56.915 | 1.00 | 40.46 | A | O |
| ATOM | 2147 | OD2 | ASP | A | 290 | 33.112 | 38.071 | 57.940 | 1.00 | 53.27 | A | O |
| ATOM | 2148 | C | ASP | A | 290 | 30.899 | 38.361 | 54.576 | 1.00 | 44.11 | A | C |
| ATOM | 2149 | O | ASP | A | 290 | 29.934 | 38.759 | 55.239 | 1.00 | 44.08 | A | O |
| ATOM | 2150 | N | ARG | A | 291 | 30.817 | 37.362 | 53.702 | 1.00 | 43.68 | A | N |
| ATOM | 2151 | CA | ARG | A | 291 | 29.572 | 36.647 | 53.464 | 1.00 | 44.07 | A | C |
| ATOM | 2152 | CB | ARG | A | 291 | 29.181 | 36.733 | 51.989 | 1.00 | 48.17 | A | C |
| ATOM | 2153 | CG | ARG | A | 291 | 28.874 | 38.146 | 51.566 | 1.00 | 53.80 | A | C |
| ATOM | 2154 | CD | ARG | A | 291 | 28.448 | 38.262 | 50.125 | 1.00 | 63.58 | A | C |
| ATOM | 2155 | NE | ARG | A | 291 | 28.267 | 39.670 | 49.781 | 1.00 | 73.84 | A | N |
| ATOM | 2156 | CZ | ARG | A | 291 | 29.236 | 40.585 | 49.826 | 1.00 | 80.35 | A | C |
| ATOM | 2157 | NH1 | ARG | A | 291 | 30.472 | 40.242 | 50.194 | 1.00 | 80.81 | A | N |
| ATOM | 2158 | NH2 | ARG | A | 291 | 28.967 | 41.853 | 49.523 | 1.00 | 82.59 | A | N |
| ATOM | 2159 | C | ARG | A | 291 | 29.798 | 35.207 | 53.868 | 1.00 | 44.80 | A | C |
| ATOM | 2160 | O | ARG | A | 291 | 29.418 | 34.282 | 53.153 | 1.00 | 46.69 | A | O |
| ATOM | 2161 | N | GLY | A | 292 | 30.409 | 35.036 | 55.039 | 1.00 | 40.06 | A | N |
| ATOM | 2162 | CA | GLY | A | 292 | 30.733 | 33.719 | 55.539 | 1.00 | 37.59 | A | C |
| ATOM | 2163 | C | GLY | A | 292 | 29.682 | 32.989 | 56.339 | 1.00 | 37.64 | A | C |
| ATOM | 2164 | O | GLY | A | 292 | 30.022 | 32.199 | 57.216 | 1.00 | 37.03 | A | O |
| ATOM | 2165 | N | GLY | A | 293 | 28.410 | 33.231 | 56.049 | 1.00 | 38.44 | A | N |
| ATOM | 2166 | CA | GLY | A | 293 | 27.370 | 32.528 | 56.777 | 1.00 | 38.57 | A | C |
| ATOM | 2167 | C | GLY | A | 293 | 26.769 | 33.285 | 57.944 | 1.00 | 41.05 | A | C |
| ATOM | 2168 | O | GLY | A | 293 | 25.587 | 33.122 | 58.238 | 1.00 | 43.55 | A | O |
| ATOM | 2169 | N | GLY | A | 294 | 27.563 | 34.112 | 58.611 | 1.00 | 38.00 | A | N |
| ATOM | 2170 | CA | GLY | A | 294 | 27.036 | 34.856 | 59.735 | 1.00 | 42.29 | A | C |
| ATOM | 2171 | C | GLY | A | 294 | 26.984 | 33.999 | 60.979 | 1.00 | 41.99 | A | C |
| ATOM | 2172 | O | GLY | A | 294 | 27.227 | 32.796 | 60.919 | 1.00 | 44.04 | A | O |
| ATOM | 2173 | N | VAL | A | 295 | 26.670 | 34.622 | 62.109 | 1.00 | 42.51 | A | N |
| ATOM | 2174 | CA | VAL | A | 295 | 26.592 | 33.925 | 63.388 | 1.00 | 42.55 | A | C |
| ATOM | 2175 | CB | VAL | A | 295 | 27.882 | 34.191 | 64.209 | 1.00 | 45.95 | A | C |
| ATOM | 2176 | CG1 | VAL | A | 295 | 27.701 | 33.774 | 65.649 | 1.00 | 48.53 | A | C |
| ATOM | 2177 | CG2 | VAL | A | 295 | 29.048 | 33.432 | 63.593 | 1.00 | 45.14 | A | C |
| ATOM | 2178 | C | VAL | A | 295 | 25.362 | 34.410 | 64.161 | 1.00 | 41.95 | A | C |
| ATOM | 2179 | O | VAL | A | 295 | 24.988 | 35.573 | 64.057 | 1.00 | 38.06 | A | O |
| ATOM | 2180 | N | PRO | A | 296 | 24.706 | 33.518 | 64.933 | 1.00 | 41.45 | A | N |
| ATOM | 2181 | CD | PRO | A | 296 | 25.048 | 32.107 | 65.152 | 1.00 | 40.11 | A | C |
| ATOM | 2182 | CA | PRO | A | 296 | 23.517 | 33.882 | 65.716 | 1.00 | 43.15 | A | C |
| ATOM | 2183 | CB | PRO | A | 296 | 23.151 | 32.578 | 66.420 | 1.00 | 43.33 | A | C |
| ATOM | 2184 | CG | PRO | A | 296 | 23.736 | 31.531 | 65.552 | 1.00 | 43.32 | A | C |
| ATOM | 2185 | C | PRO | A | 296 | 23.879 | 34.963 | 66.723 | 1.00 | 43.45 | A | C |
| ATOM | 2186 | O | PRO | A | 296 | 24.968 | 34.914 | 67.309 | 1.00 | 43.14 | A | O |
| ATOM | 2187 | N | LEU | A | 297 | 22.978 | 35.920 | 66.932 | 1.00 | 41.86 | A | N |
| ATOM | 2188 | CA | LEU | A | 297 | 23.234 | 36.997 | 67.889 | 1.00 | 44.44 | A | C |
| ATOM | 2189 | CB | LEU | A | 297 | 21.967 | 37.823 | 68.162 | 1.00 | 41.08 | A | C |
| ATOM | 2190 | CG | LEU | A | 297 | 22.190 | 39.015 | 69.121 | 1.00 | 42.04 | A | C |
| ATOM | 2191 | CD1 | LEU | A | 297 | 23.325 | 39.907 | 68.607 | 1.00 | 42.14 | A | C |
| ATOM | 2192 | CD2 | LEU | A | 297 | 20.915 | 39.827 | 69.269 | 1.00 | 40.25 | A | C |
| ATOM | 2193 | C | LEU | A | 297 | 23.745 | 36.454 | 69.218 | 1.00 | 46.23 | A | C |
| ATOM | 2194 | O | LEU | A | 297 | 24.687 | 36.989 | 69.784 | 1.00 | 46.97 | A | O |
| ATOM | 2195 | N | ARG | A | 298 | 23.132 | 35.382 | 69.709 | 1.00 | 49.93 | A | N |
| ATOM | 2196 | CA | ARG | A | 298 | 23.539 | 34.814 | 70.990 | 1.00 | 53.29 | A | C |
| ATOM | 2197 | CB | ARG | A | 298 | 22.611 | 33.664 | 71.392 | 1.00 | 60.45 | A | C |
| ATOM | 2198 | CG | ARG | A | 298 | 22.171 | 32.762 | 70.261 | 1.00 | 71.21 | A | C |
| ATOM | 2199 | CD | ARG | A | 298 | 21.368 | 31.598 | 70.818 | 1.00 | 78.12 | A | C |
| ATOM | 2200 | NE | ARG | A | 298 | 22.231 | 30.688 | 71.558 | 1.00 | 81.60 | A | N |
| ATOM | 2201 | CZ | ARG | A | 298 | 22.997 | 29.763 | 70.990 | 1.00 | 83.34 | A | C |
| ATOM | 2202 | NH1 | ARG | A | 298 | 23.001 | 29.611 | 69.671 | 1.00 | 82.00 | A | N |
| ATOM | 2203 | NH2 | ARG | A | 298 | 23.784 | 29.005 | 71.739 | 1.00 | 85.98 | A | N |
| ATOM | 2204 | C | ARG | A | 298 | 24.983 | 34.353 | 71.099 | 1.00 | 51.10 | A | C |
| ATOM | 2205 | O | ARG | A | 298 | 25.548 | 34.378 | 72.185 | 1.00 | 50.68 | A | O |
| ATOM | 2205 | N | LYS | A | 299 | 25.587 | 33.935 | 69.994 | 1.00 | 49.62 | A | N |
| ATOM | 2207 | CA | LYS | A | 299 | 26.971 | 33.486 | 70.049 | 1.00 | 51.91 | A | C |
| ATOM | 2208 | CB | LYS | A | 299 | 27.251 | 32.463 | 68.942 | 1.00 | 54.43 | A | C |
| ATOM | 2209 | CG | LYS | A | 299 | 26.423 | 31.194 | 69.092 | 1.00 | 61.02 | A | C |
| ATOM | 2210 | CD | LYS | A | 299 | 26.726 | 30.160 | 68.020 | 1.00 | 64.96 | A | C |
| ATOM | 2211 | CE | LYS | A | 299 | 25.799 | 28.954 | 68.160 | 1.00 | 68.70 | A | C |
| ATOM | 2212 | NZ | LYS | A | 299 | 25.937 | 27.989 | 67.029 | 1.00 | 68.49 | A | N |
| ATOM | 2213 | C | LYS | A | 299 | 27.944 | 34.651 | 69.936 | 1.00 | 53.04 | A | C |
| ATOM | 2214 | O | LYS | A | 299 | 29.061 | 34.579 | 70.448 | 1.00 | 50.52 | A | O |
| ATOM | 2215 | N | ILE | A | 300 | 27.527 | 35.722 | 69.261 | 1.00 | 53.95 | A | N |
| ATOM | 2216 | CA | ILE | A | 300 | 28.387 | 36.889 | 69.106 | 1.00 | 55.74 | A | C |
| ATOM | 2217 | CB | ILE | A | 300 | 27.651 | 38.050 | 68.369 | 1.00 | 54.49 | A | C |
| ATOM | 2218 | CG2 | ILE | A | 300 | 28.405 | 39.360 | 68.558 | 1.00 | 52.05 | A | C |
| ATOM | 2219 | CG1 | ILE | A | 300 | 27.538 | 37.737 | 66.877 | 1.00 | 53.33 | A | C |
| ATOM | 2220 | CD1 | ILE | A | 300 | 28.890 | 37.530 | 66.189 | 1.00 | 53.01 | A | C |
| ATOM | 2221 | C | ILE | A | 300 | 28.830 | 37.369 | 70.485 | 1.00 | 57.50 | A | C |
| ATOM | 2222 | O | ILE | A | 300 | 30.018 | 37.553 | 70.741 | 1.00 | 50.62 | A | O |
| ATOM | 2223 | N | GLU | A | 301 | 27.855 | 37.548 | 71.366 | 1.00 | 64.53 | A | N |
| ATOM | 2224 | CA | GLU | A | 301 | 28.095 | 38.009 | 72.728 | 1.00 | 73.27 | A | C |
| ATOM | 2225 | CB | GLU | A | 301 | 26.797 | 37.915 | 73.537 | 1.00 | 81.03 | A | C |
| ATOM | 2226 | CG | GLU | A | 301 | 25.502 | 38.090 | 72.722 | 1.00 | 90.68 | A | C |
| ATOM | 2227 | CD | GLU | A | 301 | 25.152 | 39.542 | 72.441 | 1.00 | 96.60 | A | C |
| ATOM | 2228 | OE1 | GLU | A | 301 | 25.963 | 40.242 | 71.800 | 1.00 | 101.10 | A | O |
| ATOM | 2229 | OE2 | GLU | A | 301 | 24.058 | 39.984 | 72.860 | 1.00 | 99.21 | A | O |
| ATOM | 2230 | C | GLU | A | 301 | 29.181 | 37.176 | 73.418 | 1.00 | 74.23 | A | C |
| ATOM | 2231 | O | GLU | A | 301 | 29.897 | 37.670 | 74.292 | 1.00 | 76.08 | A | O |
| ATOM | 2232 | N | ARG | A | 302 | 29.303 | 35.914 | 73.016 | 1.00 | 72.61 | A | N |
| ATOM | 2233 | CA | ARG | A | 302 | 30.280 | 35.005 | 73.609 | 1.00 | 70.95 | A | C |
| ATOM | 2234 | CB | ARG | A | 302 | 29.708 | 33.584 | 73.635 | 1.00 | 75.63 | A | C |
| ATOM | 2235 | CG | ARG | A | 302 | 28.320 | 33.500 | 74.265 | 1.00 | 83.68 | A | C |
| ATOM | 2236 | CD | ARG | A | 302 | 27.661 | 32.147 | 74.020 | 1.00 | 88.27 | A | C |
| ATOM | 2237 | NE | ARG | A | 302 | 28.309 | 31.063 | 74.754 | 1.00 | 92.68 | A | N |
| ATOM | 2238 | CZ | ARG | A | 302 | 28.262 | 30.920 | 76.076 | 1.00 | 94.55 | A | C |
| ATOM | 2239 | NH1 | ARG | A | 302 | 27.594 | 31.793 | 76.824 | 1.00 | 95.12 | A | N |
| ATOM | 2240 | NH2 | ARG | A | 302 | 28.880 | 29.898 | 76.651 | 1.00 | 94.82 | A | N |
| ATOM | 2241 | C | ARG | A | 302 | 31.629 | 35.001 | 72.895 | 1.00 | 67.30 | A | C |
| ATOM | 2242 | O | ARG | A | 302 | 32.591 | 34.420 | 73.396 | 1.00 | 65.42 | A | O |
| ATOM | 2243 | N | LEU | A | 303 | 31.703 | 35.648 | 71.733 | 1.00 | 61.69 | A | N |
| ATOM | 2244 | CA | LEU | A | 303 | 32.943 | 35.696 | 70.968 | 1.00 | 54.94 | A | C |
| ATOM | 2245 | CB | LEU | A | 303 | 32.682 | 36.197 | 69.535 | 1.00 | 53.68 | A | C |
| ATOM | 2246 | CG | LEU | A | 303 | 31.948 | 35.291 | 68.532 | 1.00 | 50.74 | A | C |
| ATOM | 2247 | CD1 | LEU | A | 303 | 31.855 | 35.998 | 67.201 | 1.00 | 48.99 | A | C |
| ATOM | 2248 | CD2 | LEU | A | 303 | 32.686 | 33.973 | 68.353 | 1.00 | 48.89 | A | C |
| ATOM | 2249 | C | LEU | A | 303 | 34.021 | 36.560 | 71.619 | 1.00 | 53.31 | A | C |
| ATOM | 2250 | O | LEU | A | 303 | 35.207 | 36.352 | 71.367 | 1.00 | 52.90 | A | O |
| ATOM | 2251 | N | PHE | A | 304 | 33.624 | 37.528 | 72.444 | 1.00 | 50.18 | A | N |
| ATOM | 2252 | CA | PHE | A | 304 | 34.605 | 38.396 | 73.106 | 1.00 | 50.20 | A | C |
| ATOM | 2253 | CB | PHE | A | 304 | 34.166 | 39.871 | 73.075 | 1.00 | 46.24 | A | C |
| ATOM | 2254 | CG | PHE | A | 304 | 34.238 | 40.515 | 71.709 | 1.00 | 42.35 | A | C |
| ATOM | 2255 | CD1 | PHE | A | 304 | 33.108 | 40.590 | 70.901 | 1.00 | 39.81 | A | C |
| ATOM | 2256 | CD2 | PHE | A | 304 | 35.423 | 41.082 | 71.252 | 1.00 | 40.35 | A | C |
| ATOM | 2257 | CE1 | PHE | A | 304 | 33.156 | 41.230 | 69.661 | 1.00 | 39.80 | A | C |
| ATOM | 2258 | CE2 | PHE | A | 304 | 35.476 | 41.721 | 70.011 | 1.00 | 43.77 | A | C |
| ATOM | 2259 | CZ | PHE | A | 304 | 34.336 | 41.795 | 69.220 | 1.00 | 37.55 | A | C |
| ATOM | 2260 | C | PHE | A | 304 | 34.867 | 38.007 | 74.560 | 1.00 | 51.12 | A | C |
| ATOM | 2261 | O | PHE | A | 304 | 35.511 | 38.752 | 75.305 | 1.00 | 53.90 | A | O |
| ATOM | 2262 | N | SER | A | 305 | 34.372 | 36.843 | 74.964 | 1.00 | 52.11 | A | N |
| ATOM | 2263 | CA | SER | A | 305 | 34.548 | 36.363 | 76.334 | 1.00 | 52.66 | A | C |
| ATOM | 2264 | CB | SER | A | 305 | 33.271 | 35.656 | 76.793 | 1.00 | 53.67 | A | C |
| ATOM | 2265 | OG | SER | A | 305 | 33.494 | 34.922 | 77.979 | 1.00 | 55.46 | A | O |
| ATOM | 2266 | C | SER | A | 305 | 35.743 | 35.413 | 76.455 | 1.00 | 50.85 | A | C |
| ATOM | 2267 | O | SER | A | 305 | 35.819 | 34.413 | 75.752 | 1.00 | 49.17 | A | O |
| ATOM | 2268 | N | TYR | A | 306 | 36.674 | 35.738 | 77.344 | 1.00 | 51.46 | A | N |
| ATOM | 2269 | CA | TYR | A | 306 | 37.859 | 34.911 | 77.553 | 1.00 | 53.32 | A | C |
| ATOM | 2270 | CB | TYR | A | 306 | 38.778 | 35.535 | 78.612 | 1.00 | 49.09 | A | C |
| ATOM | 2271 | CG | TYR | A | 306 | 39.274 | 36.917 | 78.266 | 1.00 | 46.60 | A | C |
| ATOM | 2272 | CD1 | TYR | A | 306 | 38.687 | 38.053 | 78.828 | 1.00 | 42.18 | A | C |
| ATOM | 2273 | CE1 | TYR | A | 306 | 39.132 | 39.333 | 78.483 | 1.00 | 40.97 | A | C |
| ATOM | 2274 | CD2 | TYR | A | 306 | 40.314 | 37.092 | 77.353 | 1.00 | 40.58 | A | C |
| ATOM | 2275 | CE2 | TYR | A | 306 | 40.757 | 38.356 | 77.006 | 1.00 | 42.16 | A | C |
| ATOM | 2276 | CZ | TYR | A | 306 | 40.168 | 39.472 | 77.572 | 1.00 | 39.05 | A | C |
| ATOM | 2277 | OH | TYR | A | 306 | 40.637 | 40.716 | 77.226 | 1.00 | 42.88 | A | O |
| ATOM | 2278 | C | TYR | A | 306 | 37.479 | 33.514 | 78.022 | 1.00 | 58.16 | A | C |
| ATOM | 2279 | O | TYR | A | 306 | 38.038 | 32.525 | 77.565 | 1.00 | 59.65 | A | O |
| ATOM | 2280 | N | MET | A | 307 | 36.521 | 33.435 | 78.937 | 1.00 | 61.71 | A | N |
| ATOM | 2281 | CA | MET | A | 307 | 36.122 | 32.148 | 79.473 | 1.00 | 67.19 | A | C |
| ATOM | 2282 | CB | MET | A | 307 | 35.131 | 32.334 | 80.616 | 1.00 | 68.20 | A | C |
| ATOM | 2283 | CG | MET | A | 307 | 34.829 | 31.032 | 81.323 | 1.00 | 74.50 | A | C |
| ATOM | 2284 | SD | MET | A | 307 | 34.129 | 31.278 | 82.948 | 1.00 | 80.19 | A | S |
| ATOM | 2285 | CE | MET | A | 307 | 32.397 | 31.353 | 82.532 | 1.00 | 81.04 | A | C |
| ATOM | 2286 | C | MET | A | 307 | 35.535 | 31.183 | 78.456 | 1.00 | 69.41 | A | C |
| ATOM | 2287 | O | MET | A | 307 | 35.998 | 30.050 | 78.316 | 1.00 | 68.66 | A | O |
| ATOM | 2288 | N | TYR | A | 308 | 34.510 | 31.635 | 77.749 | 1.00 | 71.08 | A | N |
| ATOM | 2289 | CA | TYR | A | 308 | 33.843 | 30.803 | 76.765 | 1.00 | 72.71 | A | C |
| ATOM | 2290 | CB | TYR | A | 308 | 32.504 | 31.431 | 76.409 | 1.00 | 74.23 | A | C |
| ATOM | 2291 | CG | TYR | A | 308 | 31.624 | 31.668 | 77.614 | 1.00 | 75.51 | A | C |
| ATOM | 2292 | CD1 | TYR | A | 308 | 31.079 | 32.925 | 77.856 | 1.00 | 76.60 | A | C |
| ATOM | 2293 | CE1 | TYR | A | 308 | 30.241 | 33.153 | 78.939 | 1.00 | 77.80 | A | C |
| ATOM | 2294 | CD2 | TYR | A | 308 | 31.309 | 30.631 | 78.495 | 1.00 | 75.67 | A | C |
| ATOM | 2295 | CE2 | TYR | A | 308 | 30.468 | 30.847 | 79.586 | 1.00 | 77.43 | A | C |
| ATOM | 2296 | CZ | TYR | A | 308 | 29.937 | 32.112 | 79.799 | 1.00 | 78.27 | A | C |
| ATOM | 2297 | OH | TYR | A | 308 | 29.097 | 32.345 | 80.863 | 1.00 | 79.54 | A | O |
| ATOM | 2298 | C | TYR | A | 308 | 34.650 | 30.544 | 75.498 | 1.00 | 73.16 | A | C |
| ATOM | 2299 | O | TYR | A | 308 | 34.390 | 29.577 | 74.786 | 1.00 | 73.23 | A | O |
| ATOM | 2300 | N | SER | A | 309 | 35.629 | 31.393 | 75.213 | 1.00 | 74.29 | A | N |
| ATOM | 2301 | CA | SER | A | 309 | 36.443 | 31.205 | 74.020 | 1.00 | 77.11 | A | C |
| ATOM | 2302 | CB | SER | A | 309 | 36.806 | 32.560 | 73.401 | 1.00 | 75.95 | A | C |
| ATOM | 2303 | OG | SER | A | 309 | 37.726 | 33.272 | 74.206 | 1.00 | 75.46 | A | O |
| ATOM | 2304 | C | SER | A | 309 | 37.711 | 30.427 | 74.351 | 1.00 | 80.79 | A | C |
| ATOM | 2305 | O | SER | A | 309 | 38.678 | 30.442 | 73.588 | 1.00 | 80.68 | A | O |
| ATOM | 2306 | N | THR | A | 310 | 37.699 | 29.744 | 75.492 | 1.00 | 86.62 | A | N |
| ATOM | 2307 | CA | THR | A | 310 | 38.846 | 28.956 | 75.933 | 1.00 | 93.07 | A | C |
| ATOM | 2308 | CB | THR | A | 310 | 39.275 | 29.354 | 77.370 | 1.00 | 93.59 | A | C |
| ATOM | 2309 | OG1 | THR | A | 310 | 39.696 | 30.722 | 77.377 | 1.00 | 94.34 | A | O |
| ATOM | 2310 | CG2 | THR | A | 310 | 40.428 | 28.488 | 77.856 | 1.00 | 94.77 | A | C |
| ATOM | 2311 | C | THR | A | 310 | 38.539 | 27.457 | 75.890 | 1.00 | 97.35 | A | C |
| ATOM | 2312 | O | THR | A | 310 | 37.422 | 27.029 | 76.182 | 1.00 | 96.75 | A | O |
| ATOM | 2313 | N | ALA | A | 311 | 39.546 | 26.672 | 75.517 | 1.00 | 104.45 | A | N |
| ATOM | 2314 | CA | ALA | A | 311 | 39.419 | 25.221 | 75.417 | 1.00 | 111.60 | A | C |
| ATOM | 2315 | CB | ALA | A | 311 | 38.652 | 24.859 | 74.140 | 1.00 | 111.14 | A | C |
| ATOM | 2316 | C | ALA | A | 311 | 40.818 | 24.590 | 75.399 | 1.00 | 116.93 | A | C |
| ATOM | 2317 | O | ALA | A | 311 | 41.824 | 25.300 | 75.392 | 1.00 | 116.84 | A | O |
| ATOM | 2318 | N | PRO | A | 312 | 40.903 | 23.247 | 75.396 | 1.00 | 122.15 | A | N |
| ATOM | 2319 | CD | PRO | A | 312 | 42.178 | 22.572 | 75.104 | 1.00 | 124.38 | A | C |
| ATOM | 2320 | CA | PRO | A | 312 | 39.804 | 22.274 | 75.414 | 1.00 | 125.34 | A | C |
| ATOM | 2321 | CB | PRO | A | 312 | 40.519 | 20.944 | 75.179 | 1.00 | 126.59 | A | C |
| ATOM | 2322 | CG | PRO | A | 312 | 41.717 | 21.346 | 74.364 | 1.00 | 126.58 | A | C |
| ATOM | 2323 | C | PRO | A | 312 | 39.025 | 22.286 | 76.728 | 1.00 | 127.33 | A | C |
| ATOM | 2324 | O | PRO | A | 312 | 37.793 | 22.272 | 76.733 | 1.00 | 129.13 | A | O |
| ATOM | 2325 | N | GLY | A | 327 | 41.342 | 31.135 | 70.052 | 1.00 | 106.17 | A | N |
| ATOM | 2326 | CA | GLY | A | 327 | 42.267 | 31.927 | 70.842 | 1.00 | 103.54 | A | C |
| ATOM | 2327 | C | GLY | A | 327 | 41.621 | 33.197 | 71.360 | 1.00 | 101.42 | A | C |
| ATOM | 2328 | O | GLY | A | 327 | 40.394 | 33.323 | 71.349 | 1.00 | 102.61 | A | O |
| ATOM | 2329 | N | TYR | A | 328 | 42.444 | 34.138 | 71.818 | 1.00 | 96.98 | A | N |
| ATOM | 2330 | CA | TYR | A | 328 | 41.942 | 35.410 | 72.342 | 1.00 | 90.83 | A | C |
| ATOM | 2331 | CB | TYR | A | 328 | 42.643 | 35.763 | 73.664 | 1.00 | 95.45 | A | C |
| ATOM | 2332 | CG | TYR | A | 328 | 42.353 | 34.819 | 74.817 | 1.00 | 99.48 | A | C |
| ATOM | 2333 | CD1 | TYR | A | 328 | 41.102 | 34.209 | 74.951 | 1.00 | 101.51 | A | C |
| ATOM | 2334 | CE1 | TYR | A | 328 | 40.817 | 33.375 | 76.033 | 1.00 | 102.66 | A | C |
| ATOM | 2335 | CD2 | TYR | A | 328 | 43.314 | 34.570 | 75.800 | 1.00 | 100.91 | A | C |
| ATOM | 2336 | CE2 | TYR | A | 328 | 43.036 | 33.743 | 76.886 | 1.00 | 101.80 | A | C |
| ATOM | 2337 | CZ | TYR | A | 328 | 41.785 | 33.148 | 76.997 | 1.00 | 102.59 | A | C |
| ATOM | 2338 | OH | TYR | A | 328 | 41.501 | 32.331 | 78.068 | 1.00 | 102.55 | A | O |
| ATOM | 2339 | C | TYR | A | 328 | 42.123 | 36.564 | 71.349 | 1.00 | 82.67 | A | C |
| ATOM | 2340 | O | TYR | A | 328 | 42.253 | 37.723 | 71.749 | 1.00 | 80.41 | A | O |
| ATOM | 2341 | N | GLY | A | 329 | 42.124 | 36.237 | 70.059 | 1.00 | 73.37 | A | N |
| ATOM | 2342 | CA | GLY | A | 329 | 42.294 | 37.243 | 69.028 | 1.00 | 61.96 | A | C |
| ATOM | 2343 | C | GLY | A | 329 | 41.287 | 38.375 | 69.114 | 1.00 | 55.65 | A | C |
| ATOM | 2344 | O | GLY | A | 329 | 41.663 | 39.544 | 69.094 | 1.00 | 51.75 | A | O |
| ATOM | 2345 | N | LEU | A | 330 | 40.009 | 38.034 | 69.224 | 1.00 | 50.46 | A | N |
| ATOM | 2346 | CA | LEU | A | 330 | 38.969 | 39.043 | 69.301 | 1.00 | 48.69 | A | C |
| ATOM | 2347 | CB | LEU | A | 330 | 37.593 | 38.407 | 69.071 | 1.00 | 45.00 | A | C |
| ATOM | 2348 | CG | LEU | A | 330 | 37.192 | 38.243 | 67.602 | 1.00 | 46.10 | A | C |
| ATOM | 2349 | CD1 | LEU | A | 330 | 35.828 | 37.544 | 67.497 | 1.00 | 41.90 | A | C |
| ATOM | 2350 | CD2 | LEU | A | 330 | 37.147 | 39.626 | 66.943 | 1.00 | 43.24 | A | C |
| ATOM | 2351 | C | LEU | A | 330 | 38.972 | 39.864 | 70.596 | 1.00 | 47.92 | A | C |
| ATOM | 2352 | O | LEU | A | 330 | 39.081 | 41.090 | 70.547 | 1.00 | 44.92 | A | O |
| ATOM | 2353 | N | PRO | A | 331 | 38.860 | 39.212 | 71.770 | 1.00 | 48.35 | A | N |
| ATOM | 2354 | CD | PRO | A | 331 | 38.585 | 37.799 | 72.076 | 1.00 | 48.10 | A | C |
| ATOM | 2355 | CA | PRO | A | 331 | 38.863 | 40.023 | 72.998 | 1.00 | 47.75 | A | C |
| ATOM | 2356 | CB | PRO | A | 331 | 38.778 | 38.976 | 74.120 | 1.00 | 49.33 | A | C |
| ATOM | 2357 | CG | PRO | A | 331 | 39.154 | 37.666 | 73.456 | 1.00 | 46.84 | A | C |
| ATOM | 2358 | C | PRO | A | 331 | 40.058 | 40.965 | 73.142 | 1.00 | 44.42 | A | C |
| ATOM | 2359 | O | PRO | A | 331 | 39.909 | 42.095 | 73.592 | 1.00 | 46.63 | A | O |
| ATOM | 2360 | N | ILE | A | 332 | 41.232 | 40.503 | 72.737 | 1.00 | 42.24 | A | N |
| ATOM | 2361 | CA | ILE | A | 332 | 42.439 | 41.304 | 72.819 | 1.00 | 40.73 | A | C |
| ATOM | 2362 | CB | ILE | A | 332 | 43.693 | 40.408 | 72.801 | 1.00 | 39.10 | A | C |
| ATOM | 2363 | CG2 | ILE | A | 332 | 44.924 | 41.236 | 72.514 | 1.00 | 33.90 | A | C |
| ATOM | 2364 | CG1 | ILE | A | 332 | 43.814 | 39.678 | 74.147 | 1.00 | 40.43 | A | C |
| ATOM | 2365 | CD1 | ILE | A | 332 | 44.946 | 38.668 | 74.215 | 1.00 | 45.33 | A | C |
| ATOM | 2366 | C | ILE | A | 332 | 42.532 | 42.344 | 71.705 | 1.00 | 41.49 | A | C |
| ATOM | 2367 | O | ILE | A | 332 | 43.034 | 43.446 | 71.929 | 1.00 | 43.32 | A | O |
| ATOM | 2368 | N | SER | A | 333 | 42.060 | 42.010 | 70.507 | 1.00 | 39.35 | A | N |
| ATOM | 2369 | CA | SER | A | 333 | 42.095 | 42.976 | 69.414 | 1.00 | 37.24 | A | C |
| ATOM | 2370 | CB | SER | A | 333 | 41.497 | 42.378 | 68.144 | 1.00 | 37.08 | A | C |
| ATOM | 2371 | OG | SER | A | 333 | 42.362 | 41.397 | 67.598 | 1.00 | 39.56 | A | O |
| ATOM | 2372 | C | SER | A | 333 | 41.270 | 44.190 | 69.847 | 1.00 | 38.25 | A | C |
| ATOM | 2373 | O | SER | A | 333 | 41.616 | 45.328 | 69.558 | 1.00 | 39.28 | A | O |
| ATOM | 2374 | N | ARG | A | 334 | 40.184 | 43.933 | 70.569 | 1.00 | 35.15 | A | N |
| ATOM | 2375 | CA | ARG | A | 334 | 39.320 | 44.994 | 71.042 | 1.00 | 37.10 | A | C |
| ATOM | 2376 | CB | ARG | A | 334 | 38.051 | 44.395 | 71.605 | 1.00 | 35.23 | A | C |
| ATOM | 2377 | CG | ARG | A | 334 | 37.023 | 45.429 | 71.967 | 1.00 | 39.22 | A | C |
| ATOM | 2378 | CD | ARG | A | 334 | 36.222 | 44.924 | 73.126 | 1.00 | 40.68 | A | C |
| ATOM | 2379 | NE | ARG | A | 334 | 34.854 | 44.596 | 72.781 | 1.00 | 44.82 | A | N |
| ATOM | 2380 | CZ | ARG | A | 334 | 34.080 | 43.817 | 73.530 | 1.00 | 44.06 | A | C |
| ATOM | 2381 | NH1 | ARG | A | 334 | 34.558 | 43.286 | 74.648 | 1.00 | 42.13 | A | N |
| ATOM | 2382 | NH2 | ARG | A | 334 | 32.822 | 43.599 | 73.183 | 1.00 | 42.23 | A | N |
| ATOM | 2383 | C | ARG | A | 334 | 39.983 | 45.892 | 72.100 | 1.00 | 39.10 | A | C |
| ATOM | 2384 | O | ARG | A | 334 | 39.708 | 47.092 | 72.165 | 1.00 | 38.31 | A | O |
| ATOM | 2385 | N | LEU | A | 335 | 40.841 | 45.311 | 72.937 | 1.00 | 39.33 | A | N |
| ATOM | 2386 | CA | LEU | A | 335 | 41.534 | 46.092 | 73.945 | 1.00 | 31.47 | A | C |
| ATOM | 2387 | CB | LEU | A | 335 | 42.296 | 45.173 | 74.901 | 1.00 | 32.46 | A | C |
| ATOM | 2388 | CG | LEU | A | 335 | 41.366 | 44.334 | 75.787 | 1.00 | 32.96 | A | C |
| ATOM | 2389 | CD1 | LEU | A | 335 | 42.162 | 43.424 | 76.686 | 1.00 | 33.31 | A | C |
| ATOM | 2390 | CD2 | LEU | A | 335 | 40.496 | 45.262 | 76.609 | 1.00 | 33.94 | A | C |
| ATOM | 2391 | C | LEU | A | 335 | 42.487 | 47.012 | 73.196 | 1.00 | 31.90 | A | C |
| ATOM | 2392 | O | LEU | A | 335 | 42.685 | 48.151 | 73.594 | 1.00 | 33.49 | A | O |
| ATOM | 2393 | N | TYR | A | 336 | 43.070 | 46.535 | 72.096 | 1.00 | 31.40 | A | N |
| ATOM | 2394 | CA | TYR | A | 336 | 43.970 | 47.391 | 71.323 | 1.00 | 37.23 | A | C |
| ATOM | 2395 | CB | TYR | A | 336 | 44.619 | 46.626 | 70.169 | 1.00 | 41.28 | A | C |
| ATOM | 2396 | CG | TYR | A | 336 | 45.872 | 45.897 | 70.549 | 1.00 | 48.65 | A | C |
| ATOM | 2397 | CD1 | TYR | A | 336 | 45.863 | 44.520 | 70.738 | 1.00 | 51.74 | A | C |
| ATOM | 2398 | CE1 | TYR | A | 336 | 47.015 | 43.836 | 71.107 | 1.00 | 53.88 | A | C |
| ATOM | 2399 | CD2 | TYR | A | 336 | 47.068 | 46.583 | 70.739 | 1.00 | 50.43 | A | C |
| ATOM | 2400 | CE2 | TYR | A | 336 | 48.229 | 45.910 | 71.113 | 1.00 | 53.05 | A | C |
| ATOM | 2401 | CZ | TYR | A | 336 | 48.191 | 44.532 | 71.294 | 1.00 | 52.52 | A | C |
| ATOM | 2402 | OH | TYR | A | 336 | 49.319 | 43.844 | 71.668 | 1.00 | 54.37 | A | O |
| ATOM | 2403 | C | TYR | A | 336 | 43.220 | 48.586 | 70.733 | 1.00 | 36.91 | A | C |
| ATOM | 2404 | O | TYR | A | 336 | 43.788 | 49.669 | 70.578 | 1.00 | 36.84 | A | O |
| ATOM | 2405 | N | ALA | A | 337 | 41.953 | 48.370 | 70.382 | 1.00 | 35.92 | A | N |
| ATOM | 2406 | CA | ALA | A | 337 | 41.122 | 49.425 | 69.820 | 1.00 | 38.06 | A | C |
| ATOM | 2407 | CB | ALA | A | 337 | 39.826 | 48.834 | 69.273 | 1.00 | 35.78 | A | C |
| ATOM | 2408 | C | ALA | A | 337 | 40.814 | 50.441 | 70.919 | 1.00 | 37.61 | A | C |
| ATOM | 2409 | O | ALA | A | 337 | 40.956 | 51.653 | 70.712 | 1.00 | 32.49 | A | O |
| ATOM | 2410 | N | LYS | A | 338 | 40.417 | 49.930 | 72.088 | 1.00 | 35.75 | A | N |
| ATOM | 2411 | CA | LYS | A | 338 | 40.064 | 50.759 | 73.250 | 1.00 | 37.01 | A | C |
| ATOM | 2412 | CB | LYS | A | 338 | 39.413 | 49.910 | 74.351 | 1.00 | 37.48 | A | C |
| ATOM | 2413 | CG | LYS | A | 338 | 38.022 | 49.383 | 74.060 | 1.00 | 37.11 | A | C |
| ATOM | 2414 | CD | LYS | A | 338 | 37.503 | 48.642 | 75.274 | 1.00 | 41.23 | A | C |
| ATOM | 2415 | CE | LYS | A | 338 | 36.233 | 47.857 | 74.973 | 1.00 | 42.13 | A | C |
| ATOM | 2416 | NZ | LYS | A | 338 | 35.015 | 48.696 | 74.913 | 1.00 | 48.87 | A | N |
| ATOM | 2417 | C | LYS | A | 338 | 41.252 | 51.479 | 73.879 | 1.00 | 37.80 | A | C |
| ATOM | 2418 | O | LYS | A | 338 | 41.101 | 52.552 | 74.455 | 1.00 | 34.04 | A | O |
| ATOM | 2419 | N | TYR | A | 339 | 42.432 | 50.883 | 73.779 | 1.00 | 37.01 | A | N |
| ATOM | 2420 | CA | TYR | A | 339 | 43.618 | 51.473 | 74.387 | 1.00 | 39.39 | A | C |
| ATOM | 2421 | CB | TYR | A | 339 | 44.855 | 50.676 | 73.951 | 1.00 | 36.06 | A | C |
| ATOM | 2422 | CG | TYR | A | 339 | 46.161 | 51.247 | 74.414 | 1.00 | 31.24 | A | C |
| ATOM | 2423 | CD1 | TYR | A | 339 | 46.328 | 51.709 | 75.724 | 1.00 | 34.51 | A | C |
| ATOM | 2424 | CE1 | TYR | A | 339 | 47.547 | 52.241 | 76.149 | 1.00 | 27.10 | A | C |
| ATOM | 2425 | CD2 | TYR | A | 339 | 47.245 | 51.321 | 73.545 | 1.00 | 34.06 | A | C |
| ATOM | 2426 | CE2 | TYR | A | 339 | 48.468 | 51.842 | 73.965 | 1.00 | 34.69 | A | C |
| ATOM | 2427 | CZ | TYR | A | 339 | 48.608 | 52.300 | 75.267 | 1.00 | 36.22 | A | C |
| ATOM | 2428 | OH | TYR | A | 339 | 49.821 | 52.814 | 75.680 | 1.00 | 45.28 | A | O |
| ATOM | 2429 | C | TYR | A | 339 | 43.759 | 52.972 | 74.085 | 1.00 | 39.82 | A | C |
| ATOM | 2430 | O | TYR | A | 339 | 44.124 | 53.758 | 74.965 | 1.00 | 38.38 | A | O |
| ATOM | 2431 | N | PHE | A | 340 | 43.452 | 53.366 | 72.852 | 1.00 | 37.29 | A | N |
| ATOM | 2432 | CA | PHE | A | 340 | 43.517 | 54.772 | 72.451 | 1.00 | 36.16 | A | C |
| ATOM | 2433 | CB | PHE | A | 340 | 44.408 | 54.938 | 71.216 | 1.00 | 38.39 | A | C |
| ATOM | 2434 | CG | PHE | A | 340 | 45.875 | 54.992 | 71.534 | 1.00 | 42.43 | A | C |
| ATOM | 2435 | CD1 | PHE | A | 340 | 46.398 | 56.048 | 72.281 | 1.00 | 43.08 | A | C |
| ATOM | 2436 | CD2 | PHE | A | 340 | 46.729 | 53.983 | 71.113 | 1.00 | 42.23 | A | C |
| ATOM | 2437 | CE1 | PHE | A | 340 | 47.754 | 56.094 | 72.604 | 1.00 | 43.28 | A | C |
| ATOM | 2438 | CE2 | PHE | A | 340 | 48.081 | 54.018 | 71.427 | 1.00 | 44.09 | A | C |
| ATOM | 2439 | CZ | PHE | A | 340 | 48.596 | 55.078 | 72.176 | 1.00 | 44.81 | A | C |
| ATOM | 2440 | C | PHE | A | 340 | 42.129 | 55.360 | 72.178 | 1.00 | 36.13 | A | C |
| ATOM | 2441 | O | PHE | A | 340 | 41.927 | 56.100 | 71.211 | 1.00 | 35.60 | A | O |
| ATOM | 2442 | N | GLN | A | 341 | 41.175 | 55.010 | 73.037 | 1.00 | 35.88 | A | N |
| ATOM | 2443 | CA | GLN | A | 341 | 39.801 | 55.501 | 72.952 | 1.00 | 36.74 | A | C |
| ATOM | 2444 | CB | GLN | A | 341 | 39.789 | 57.044 | 72.959 | 1.00 | 35.49 | A | C |
| ATOM | 2445 | CG | GLN | A | 341 | 40.567 | 57.715 | 74.085 | 1.00 | 34.44 | A | C |
| ATOM | 2446 | CD | GLN | A | 341 | 40.063 | 57.354 | 75.464 | 1.00 | 39.98 | A | C |
| ATOM | 2447 | OE1 | GLN | A | 341 | 38.861 | 57.402 | 75.731 | 1.00 | 42.68 | A | O |
| ATOM | 2448 | NE2 | GLN | A | 341 | 40.986 | 56.994 | 76.360 | 1.00 | 39.62 | A | N |
| ATOM | 2449 | C | GLN | A | 341 | 38.978 | 55.001 | 71.750 | 1.00 | 36.54 | A | C |
| ATOM | 2450 | O | GLN | A | 341 | 37.965 | 55.599 | 71.404 | 1.00 | 39.15 | A | O |
| ATOM | 2451 | N | GLY | A | 342 | 39.402 | 53.919 | 71.111 | 1.00 | 37.28 | A | N |
| ATOM | 2452 | CA | GLY | A | 342 | 38.638 | 53.395 | 69.994 | 1.00 | 29.69 | A | C |
| ATOM | 2453 | C | GLY | A | 342 | 37.768 | 52.251 | 70.468 | 1.00 | 32.87 | A | C |
| ATOM | 2454 | O | GLY | A | 342 | 37.496 | 52.144 | 71.665 | 1.00 | 37.62 | A | O |
| ATOM | 2455 | N | ASP | A | 343 | 37.326 | 51.394 | 69.546 | 1.00 | 35.14 | A | N |
| ATOM | 2456 | CA | ASP | A | 343 | 36.498 | 50.235 | 69.893 | 1.00 | 30.31 | A | C |
| ATOM | 2457 | CB | ASP | A | 343 | 35.047 | 50.679 | 70.175 | 1.00 | 26.44 | A | C |
| ATOM | 2458 | CG | ASP | A | 343 | 34.241 | 49.642 | 70.993 | 1.00 | 33.63 | A | C |
| ATOM | 2459 | OD1 | ASP | A | 343 | 34.761 | 48.559 | 71.333 | 1.00 | 35.88 | A | O |
| ATOM | 2460 | OD2 | ASP | A | 343 | 33.065 | 49.903 | 71.299 | 1.00 | 35.63 | A | O |
| ATOM | 2461 | C | ASP | A | 343 | 36.538 | 49.242 | 68.726 | 1.00 | 31.38 | A | C |
| ATOM | 2462 | O | ASP | A | 343 | 37.081 | 49.546 | 67.656 | 1.00 | 32.71 | A | O |
| ATOM | 2463 | N | LEU | A | 344 | 36.011 | 48.041 | 68.943 | 1.00 | 26.64 | A | N |
| ATOM | 2464 | CA | LEU | A | 344 | 35.953 | 47.044 | 67.881 | 1.00 | 29.81 | A | C |
| ATOM | 2465 | CB | LEU | A | 344 | 37.103 | 46.056 | 67.990 | 1.00 | 31.21 | A | C |
| ATOM | 2466 | CG | LEU | A | 344 | 37.267 | 45.045 | 66.845 | 1.00 | 32.81 | A | C |
| ATOM | 2467 | CD1 | LEU | A | 344 | 38.687 | 44.512 | 66.860 | 1.00 | 32.78 | A | C |
| ATOM | 2468 | CD2 | LEU | A | 344 | 36.254 | 43.914 | 66.987 | 1.00 | 31.03 | A | C |
| ATOM | 2469 | C | LEU | A | 344 | 34.627 | 46.342 | 68.086 | 1.00 | 30.67 | A | C |
| ATOM | 2470 | O | LEU | A | 344 | 34.388 | 45.780 | 69.142 | 1.00 | 31.64 | A | O |
| ATOM | 2471 | N | GLN | A | 345 | 33.746 | 46.390 | 67.091 | 1.00 | 30.88 | A | N |
| ATOM | 2472 | CA | GLN | A | 345 | 32.447 | 45.762 | 67.277 | 1.00 | 31.33 | A | C |
| ATOM | 2473 | CB | GLN | A | 345 | 31.374 | 46.842 | 67.369 | 1.00 | 27.91 | A | C |
| ATOM | 2474 | CG | GLN | A | 345 | 31.732 | 47.921 | 68.381 | 1.00 | 36.19 | A | C |
| ATOM | 2475 | CD | GLN | A | 345 | 30.709 | 49.045 | 68.468 | 1.00 | 39.76 | A | C |
| ATOM | 2476 | OE1 | GLN | A | 345 | 29.936 | 49.275 | 67.548 | 1.00 | 39.01 | A | O |
| ATOM | 2477 | NE2 | GLN | A | 345 | 30.731 | 49.769 | 69.575 | 1.00 | 43.86 | A | N |
| ATOM | 2478 | C | GLN | A | 345 | 32.092 | 44.784 | 66.178 | 1.00 | 29.45 | A | C |
| ATOM | 2479 | O | GLN | A | 345 | 32.609 | 44.860 | 65.068 | 1.00 | 28.51 | A | O |
| ATOM | 2480 | N | LEU | A | 346 | 31.203 | 43.858 | 66.499 | 1.00 | 30.41 | A | N |
| ATOM | 2481 | CA | LEU | A | 346 | 30.767 | 42.875 | 65.534 | 1.00 | 32.42 | A | C |
| ATOM | 2482 | CB | LEU | A | 346 | 31.228 | 41.464 | 65.920 | 1.00 | 33.17 | A | C |
| ATOM | 2483 | CG | LEU | A | 346 | 32.719 | 41.126 | 65.963 | 1.00 | 42.37 | A | C |
| ATOM | 2484 | CD1 | LEU | A | 346 | 32.860 | 39.635 | 66.270 | 1.00 | 42.20 | A | C |
| ATOM | 2485 | CD2 | LEU | A | 346 | 33.383 | 41.445 | 64.635 | 1.00 | 39.76 | A | C |
| ATOM | 2486 | C | LEU | A | 346 | 29.258 | 42.844 | 65.460 | 1.00 | 32.00 | A | C |
| ATOM | 2487 | O | LEU | A | 346 | 28.563 | 43.048 | 66.455 | 1.00 | 30.45 | A | O |
| ATOM | 2488 | N | PHE | A | 347 | 28.770 | 42.563 | 64.262 | 1.00 | 32.06 | A | N |
| ATOM | 2489 | CA | PHE | A | 347 | 27.350 | 42.402 | 64.006 | 1.00 | 38.23 | A | C |
| ATOM | 2490 | CB | PHE | A | 347 | 26.642 | 43.753 | 63.899 | 1.00 | 42.56 | A | C |
| ATOM | 2491 | CG | PHE | A | 347 | 27.495 | 44.842 | 63.363 | 1.00 | 48.88 | A | C |
| ATOM | 2492 | CD1 | PHE | A | 347 | 27.546 | 45.093 | 61.998 | 1.00 | 56.82 | A | C |
| ATOM | 2493 | CD2 | PHE | A | 347 | 28.255 | 45.624 | 64.226 | 1.00 | 52.47 | A | C |
| ATOM | 2494 | CE1 | PHE | A | 347 | 28.349 | 46.119 | 61.494 | 1.00 | 61.97 | A | C |
| ATOM | 2495 | CE2 | PHE | A | 347 | 29.059 | 46.640 | 63.748 | 1.00 | 56.14 | A | C |
| ATOM | 2496 | CZ | PHE | A | 347 | 29.110 | 46.894 | 62.372 | 1.00 | 61.46 | A | C |
| ATOM | 2497 | C | PHE | A | 347 | 27.179 | 41.591 | 62.728 | 1.00 | 36.98 | A | C |
| ATOM | 2498 | O | PHE | A | 347 | 27.647 | 41.975 | 61.660 | 1.00 | 37.08 | A | O |
| ATOM | 2499 | N | SER | A | 348 | 26.549 | 40.435 | 62.835 | 1.00 | 35.77 | A | N |
| ATOM | 2500 | CA | SER | A | 348 | 26.356 | 39.649 | 61.630 | 1.00 | 39.28 | A | C |
| ATOM | 2501 | CB | SER | A | 348 | 27.121 | 38.329 | 61.709 | 1.00 | 39.29 | A | C |
| ATOM | 2502 | OG | SER | A | 348 | 26.626 | 37.526 | 62.742 | 1.00 | 35.92 | A | O |
| ATOM | 2503 | C | SER | A | 348 | 24.886 | 39.375 | 61.418 | 1.00 | 36.97 | A | C |
| ATOM | 2504 | O | SER | A | 348 | 24.044 | 39.791 | 62.219 | 1.00 | 36.96 | A | O |
| ATOM | 2505 | N | MET | A | 349 | 24.602 | 38.680 | 60.325 | 1.00 | 37.28 | A | N |
| ATOM | 2506 | CA | MET | A | 349 | 23.252 | 38.306 | 59.928 | 1.00 | 38.46 | A | C |
| ATOM | 2507 | CB | MET | A | 349 | 22.907 | 38.899 | 58.565 | 1.00 | 42.20 | A | C |
| ATOM | 2508 | CG | MET | A | 349 | 23.445 | 40.274 | 58.313 | 1.00 | 44.70 | A | C |
| ATOM | 2509 | SD | MET | A | 349 | 22.278 | 41.495 | 58.772 | 1.00 | 54.61 | A | S |
| ATOM | 2510 | CE | MET | A | 349 | 21.080 | 41.373 | 57.467 | 1.00 | 43.61 | A | C |
| ATOM | 2511 | C | MET | A | 349 | 23.336 | 36.815 | 59.750 | 1.00 | 37.71 | A | C |
| ATOM | 2512 | O | MET | A | 349 | 23.970 | 36.350 | 58.795 | 1.00 | 37.40 | A | O |
| ATOM | 2513 | N | GLU | A | 350 | 22.704 | 36.052 | 60.629 | 1.00 | 36.08 | A | N |
| ATOM | 2514 | CA | GLU | A | 350 | 22.793 | 34.612 | 60.474 | 1.00 | 37.07 | A | C |
| ATOM | 2515 | CB | GLU | A | 350 | 22.015 | 33.888 | 61.558 | 1.00 | 39.11 | A | C |
| ATOM | 2516 | CG | GLU | A | 350 | 22.517 | 32.477 | 61.776 | 1.00 | 41.65 | A | C |
| ATOM | 2517 | CD | GLU | A | 350 | 21.581 | 31.656 | 62.621 | 1.00 | 43.67 | A | C |
| ATOM | 2518 | OE1 | GLU | A | 350 | 22.002 | 30.577 | 63.085 | 1.00 | 47.32 | A | O |
| ATOM | 2519 | OE2 | GLU | A | 350 | 20.422 | 32.087 | 62.815 | 1.00 | 43.16 | A | O |
| ATOM | 2520 | C | GLU | A | 350 | 22.236 | 34.232 | 59.116 | 1.00 | 34.86 | A | C |
| ATOM | 2521 | O | GLU | A | 350 | 21.142 | 34.664 | 58.745 | 1.00 | 32.20 | A | O |
| ATOM | 2522 | N | GLY | A | 351 | 23.001 | 33.427 | 58.382 | 1.00 | 35.78 | A | N |
| ATOM | 2523 | CA | GLY | A | 351 | 22.585 | 32.983 | 57.065 | 1.00 | 37.01 | A | C |
| ATOM | 2524 | C | GLY | A | 351 | 23.125 | 33.852 | 55.946 | 1.00 | 38.03 | A | C |
| ATOM | 2525 | O | GLY | A | 351 | 22.803 | 33.631 | 54.776 | 1.00 | 38.56 | A | O |
| ATOM | 2526 | N | PHE | A | 352 | 23.961 | 34.829 | 56.290 | 1.00 | 36.96 | A | N |
| ATOM | 2527 | CA | PHE | A | 352 | 24.508 | 35.729 | 55.281 | 1.00 | 37.10 | A | C |
| ATOM | 2528 | CB | PHE | A | 352 | 23.684 | 37.016 | 55.201 | 1.00 | 37.36 | A | C |
| ATOM | 2529 | CG | PHE | A | 352 | 24.129 | 37.947 | 54.106 | 1.00 | 41.28 | A | C |
| ATOM | 2530 | CD1 | PHE | A | 352 | 23.981 | 37.584 | 52.770 | 1.00 | 44.83 | A | C |
| ATOM | 2531 | CD2 | PHE | A | 352 | 24.750 | 39.156 | 54.402 | 1.00 | 44.89 | A | C |
| ATOM | 2532 | CE1 | PHE | A | 352 | 24.450 | 38.404 | 51.742 | 1.00 | 42.37 | A | C |
| ATOM | 2533 | CE2 | PHE | A | 352 | 25.224 | 39.985 | 53.375 | 1.00 | 43.98 | A | C |
| ATOM | 2534 | CZ | PHE | A | 352 | 25.071 | 39.599 | 52.044 | 1.00 | 42.73 | A | C |
| ATOM | 2535 | C | PHE | A | 352 | 25.971 | 36.103 | 55.481 | 1.00 | 39.28 | A | C |
| ATOM | 2536 | O | PHE | A | 352 | 26.826 | 35.703 | 54.688 | 1.00 | 42.12 | A | O |
| ATOM | 2537 | N | GLY | A | 353 | 26.267 | 36.878 | 56.521 | 1.00 | 37.82 | A | N |
| ATOM | 2538 | CA | GLY | A | 353 | 27.643 | 37.291 | 56.737 | 1.00 | 37.45 | A | C |
| ATOM | 2539 | C | GLY | A | 353 | 27.857 | 38.097 | 57.998 | 1.00 | 37.43 | A | C |
| ATOM | 2540 | O | GLY | A | 353 | 26.975 | 38.199 | 58.830 | 1.00 | 43.48 | A | O |
| ATOM | 2541 | N | THR | A | 354 | 29.031 | 38.690 | 58.136 | 1.00 | 37.03 | A | N |
| ATOM | 2542 | CA | THR | A | 354 | 29.353 | 39.456 | 59.327 | 1.00 | 35.17 | A | C |
| ATOM | 2543 | CB | THR | A | 354 | 30.225 | 38.618 | 60.279 | 1.00 | 29.92 | A | C |
| ATOM | 2544 | OG1 | THR | A | 354 | 29.557 | 37.388 | 60.563 | 1.00 | 28.96 | A | O |
| ATOM | 2545 | CG2 | THR | A | 354 | 30.492 | 39.370 | 61.599 | 1.00 | 25.53 | A | C |
| ATOM | 2546 | C | THR | A | 354 | 30.114 | 40.734 | 59.032 | 1.00 | 37.44 | A | C |
| ATOM | 2547 | O | THR | A | 354 | 30.966 | 40.769 | 58.139 | 1.00 | 38.08 | A | O |
| ATOM | 2548 | N | ASP | A | 355 | 29.795 | 41.782 | 59.782 | 1.00 | 34.01 | A | N |
| ATOM | 2549 | CA | ASP | A | 355 | 30.507 | 43.049 | 59.656 | 1.00 | 36.83 | A | C |
| ATOM | 2550 | CB | ASP | A | 355 | 29.548 | 44.221 | 59.484 | 1.00 | 36.18 | A | C |
| ATOM | 2551 | CG | ASP | A | 355 | 28.977 | 44.308 | 58.070 | 1.00 | 44.34 | A | C |
| ATOM | 2552 | OD1 | ASP | A | 355 | 29.171 | 43.352 | 57.294 | 1.00 | 44.47 | A | O |
| ATOM | 2553 | OD2 | ASP | A | 355 | 28.330 | 45.327 | 57.735 | 1.00 | 45.17 | A | O |
| ATOM | 2554 | C | ASP | A | 355 | 31.288 | 43.219 | 60.945 | 1.00 | 35.09 | A | C |
| ATOM | 2555 | O | ASP | A | 355 | 30.781 | 42.941 | 62.025 | 1.00 | 36.87 | A | O |
| ATOM | 2556 | N | ALA | A | 356 | 32.540 | 43.624 | 60.818 | 1.00 | 31.11 | A | N |
| ATOM | 2557 | CA | ALA | A | 356 | 33.384 | 43.850 | 61.968 | 1.00 | 30.16 | A | C |
| ATOM | 2558 | CB | ALA | A | 356 | 34.565 | 42.905 | 61.960 | 1.00 | 30.14 | A | C |
| ATOM | 2559 | C | ALA | A | 356 | 33.839 | 45.270 | 61.744 | 1.00 | 30.07 | A | C |
| ATOM | 2560 | O | ALA | A | 356 | 34.208 | 45.630 | 60.634 | 1.00 | 31.21 | A | O |
| ATOM | 2561 | N | VAL | A | 357 | 33.783 | 46.090 | 62.782 | 1.00 | 29.07 | A | N |
| ATOM | 2562 | CA | VAL | A | 357 | 34.180 | 47.466 | 62.632 | 1.00 | 32.24 | A | C |
| ATOM | 2563 | CB | VAL | A | 357 | 32.942 | 48.394 | 62.625 | 1.00 | 33.55 | A | C |
| ATOM | 2564 | CG1 | VAL | A | 357 | 33.363 | 49.827 | 62.379 | 1.00 | 35.33 | A | C |
| ATOM | 2565 | CG2 | VAL | A | 357 | 31.966 | 47.959 | 61.541 | 1.00 | 38.31 | A | C |
| ATOM | 2566 | C | VAL | A | 357 | 35.126 | 47.916 | 63.726 | 1.00 | 31.22 | A | C |
| ATOM | 2567 | O | VAL | A | 357 | 34.887 | 47.679 | 64.912 | 1.00 | 33.89 | A | O |
| ATOM | 2568 | N | ILE | A | 358 | 36.204 | 48.563 | 63.302 | 1.00 | 28.76 | A | N |
| ATOM | 2569 | CA | ILE | A | 358 | 37.199 | 49.121 | 64.195 | 1.00 | 31.35 | A | C |
| ATOM | 2570 | CB | ILE | A | 358 | 38.631 | 48.828 | 63.704 | 1.00 | 30.05 | A | C |
| ATOM | 2571 | CG2 | ILE | A | 358 | 39.642 | 49.479 | 64.649 | 1.00 | 29.50 | A | C |
| ATOM | 2572 | CG1 | ILE | A | 358 | 38.871 | 47.315 | 63.632 | 1.00 | 27.15 | A | C |
| ATOM | 2573 | CD1 | ILE | A | 358 | 40.155 | 46.943 | 62.894 | 1.00 | 28.38 | A | C |
| ATOM | 2574 | C | ILE | A | 358 | 37.032 | 50.643 | 64.199 | 1.00 | 33.48 | A | C |
| ATOM | 2575 | O | ILE | A | 358 | 37.120 | 51.281 | 63.156 | 1.00 | 33.58 | A | O |
| ATOM | 2576 | N | TYR | A | 359 | 36.781 | 51.210 | 65.373 | 1.00 | 34.55 | A | N |
| ATOM | 2577 | CA | TYR | A | 359 | 36.634 | 52.652 | 65.548 | 1.00 | 33.20 | A | C |
| ATOM | 2578 | CB | TYR | A | 359 | 35.455 | 52.984 | 66.454 | 1.00 | 32.88 | A | C |
| ATOM | 2579 | CG | TYR | A | 359 | 34.131 | 52.583 | 65.881 | 1.00 | 32.98 | A | C |
| ATOM | 2580 | CD1 | TYR | A | 359 | 33.466 | 51.452 | 66.350 | 1.00 | 23.84 | A | C |
| ATOM | 2581 | CE1 | TYR | A | 359 | 32.257 | 51.053 | 65.795 | 1.00 | 31.40 | A | C |
| ATOM | 2582 | CD2 | TYR | A | 359 | 33.556 | 53.317 | 64.832 | 1.00 | 29.40 | A | C |
| ATOM | 2583 | CE2 | TYR | A | 359 | 32.346 | 52.925 | 64.261 | 1.00 | 31.43 | A | C |
| ATOM | 2584 | CZ | TYR | A | 359 | 31.702 | 51.798 | 64.749 | 1.00 | 32.82 | A | C |
| ATOM | 2585 | OH | TYR | A | 359 | 30.491 | 51.438 | 64.218 | 1.00 | 35.01 | A | O |
| ATOM | 2586 | C | TYR | A | 359 | 37.877 | 53.192 | 66.219 | 1.00 | 34.85 | A | C |
| ATOM | 2587 | O | TYR | A | 359 | 38.201 | 52.779 | 67.325 | 1.00 | 38.03 | A | O |
| ATOM | 2588 | N | LEU | A | 360 | 38.565 | 54.106 | 65.542 | 1.00 | 36.15 | A | N |
| ATOM | 2589 | CA | LEU | A | 360 | 39.770 | 54.747 | 66.065 | 1.00 | 35.85 | A | C |
| ATOM | 2590 | CB | LEU | A | 360 | 40.965 | 54.480 | 65.157 | 1.00 | 27.65 | A | C |
| ATOM | 2591 | CG | LEU | A | 360 | 41.289 | 53.060 | 64.706 | 1.00 | 26.77 | A | C |
| ATOM | 2592 | CD1 | LEU | A | 360 | 42.408 | 53.163 | 63.663 | 1.00 | 22.95 | A | C |
| ATOM | 2593 | CD2 | LEU | A | 360 | 41.720 | 52.182 | 65.921 | 1.00 | 23.02 | A | C |
| ATOM | 2594 | C | LEU | A | 360 | 39.501 | 56.250 | 66.044 | 1.00 | 37.12 | A | C |
| ATOM | 2595 | O | LEU | A | 360 | 38.628 | 56.716 | 65.289 | 1.00 | 34.88 | A | O |
| ATOM | 2596 | N | LYS | A | 361 | 40.236 | 57.005 | 66.858 | 1.00 | 34.27 | A | N |
| ATOM | 2597 | CA | LYS | A | 361 | 40.066 | 58.449 | 66.869 | 1.00 | 35.74 | A | C |
| ATOM | 2598 | CB | LYS | A | 361 | 40.641 | 59.065 | 68.136 | 1.00 | 35.79 | A | C |
| ATOM | 2599 | CG | LYS | A | 361 | 39.909 | 58.701 | 69.388 | 1.00 | 37.90 | A | C |
| ATOM | 2600 | CD | LYS | A | 361 | 38.478 | 59.216 | 69.386 | 1.00 | 41.63 | A | C |
| ATOM | 2601 | CE | LYS | A | 361 | 37.924 | 59.173 | 70.812 | 1.00 | 40.19 | A | C |
| ATOM | 2602 | NZ | LYS | A | 361 | 36.481 | 59.494 | 70.900 | 1.00 | 41.92 | A | N |
| ATOM | 2603 | C | LYS | A | 361 | 40.844 | 58.973 | 65.676 | 1.00 | 38.66 | A | C |
| ATOM | 2604 | O | LYS | A | 361 | 41.957 | 58.510 | 65.415 | 1.00 | 37.90 | A | O |
| ATOM | 2605 | N | ALA | A | 362 | 40.267 | 59.933 | 64.954 | 1.00 | 39.94 | A | N |
| ATOM | 2606 | CA | ALA | A | 362 | 40.933 | 60.517 | 63.792 | 1.00 | 43.21 | A | C |
| ATOM | 2607 | CB | ALA | A | 362 | 39.897 | 61.229 | 62.885 | 1.00 | 42.70 | A | C |
| ATOM | 2608 | C | ALA | A | 362 | 42.040 | 61.503 | 64.199 | 1.00 | 44.40 | A | C |
| ATOM | 2609 | O | ALA | A | 362 | 43.065 | 61.594 | 63.527 | 1.00 | 46.69 | A | O |
| ATOM | 2610 | N | LEU | A | 363 | 41.835 | 62.228 | 65.297 | 1.00 | 44.81 | A | N |
| ATOM | 2611 | CA | LEU | A | 363 | 42.810 | 63.220 | 65.765 | 1.00 | 48.01 | A | C |
| ATOM | 2612 | CB | LEU | A | 363 | 42.076 | 64.457 | 66.289 | 1.00 | 48.66 | A | C |
| ATOM | 2613 | CG | LEU | A | 363 | 41.022 | 65.100 | 65.374 | 1.00 | 53.96 | A | C |
| ATOM | 2614 | CD1 | LEU | A | 363 | 40.279 | 66.175 | 66.147 | 1.00 | 51.64 | A | C |
| ATOM | 2615 | CD2 | LEU | A | 363 | 41.671 | 65.686 | 64.135 | 1.00 | 52.57 | A | C |
| ATOM | 2616 | C | LEU | A | 363 | 43.763 | 62.701 | 66.855 | 1.00 | 48.63 | A | C |
| ATOM | 2617 | O | LEU | A | 363 | 43.338 | 62.071 | 67.830 | 1.00 | 48.05 | A | O |
| ATOM | 2618 | N | SER | A | 364 | 45.051 | 62.979 | 66.692 | 1.00 | 48.16 | A | N |
| ATOM | 2619 | CA | SER | A | 364 | 46.042 | 62.537 | 67.666 | 1.00 | 52.56 | A | C |
| ATOM | 2620 | CB | SER | A | 364 | 47.452 | 62.907 | 67.199 | 1.00 | 48.97 | A | C |
| ATOM | 2621 | OG | SER | A | 364 | 47.551 | 64.296 | 66.970 | 1.00 | 55.11 | A | O |
| ATOM | 2622 | C | SER | A | 364 | 45.739 | 63.190 | 69.004 | 1.00 | 54.15 | A | C |
| ATOM | 2623 | O | SER | A | 364 | 46.156 | 62.715 | 70.056 | 1.00 | 56.29 | A | O |
| ATOM | 2624 | N | THR | A | 365 | 44.995 | 64.282 | 68.958 | 1.00 | 56.50 | A | N |
| ATOM | 2625 | CA | THR | A | 365 | 44.619 | 64.991 | 70.167 | 1.00 | 59.01 | A | C |
| ATOM | 2626 | CB | THR | A | 365 | 43.841 | 66.265 | 69.830 | 1.00 | 62.19 | A | C |
| ATOM | 2627 | OG1 | THR | A | 365 | 44.534 | 66.995 | 68.812 | 1.00 | 66.29 | A | O |
| ATOM | 2628 | CG2 | THR | A | 365 | 43.686 | 67.128 | 71.071 | 1.00 | 65.89 | A | C |
| ATOM | 2629 | C | THR | A | 365 | 43.702 | 64.123 | 71.024 | 1.00 | 58.25 | A | C |
| ATOM | 2630 | O | THR | A | 365 | 43.807 | 64.106 | 72.251 | 1.00 | 59.08 | A | O |
| ATOM | 2631 | N | ASP | A | 366 | 42.793 | 63.415 | 70.358 | 1.00 | 57.53 | A | N |
| ATOM | 2632 | CA | ASP | A | 366 | 41.812 | 62.563 | 71.024 | 1.00 | 54.61 | A | C |
| ATOM | 2633 | CB | ASP | A | 366 | 40.550 | 62.439 | 70.151 | 1.00 | 57.16 | A | C |
| ATOM | 2634 | CG | ASP | A | 366 | 39.912 | 63.784 | 69.831 | 1.00 | 59.06 | A | C |
| ATOM | 2635 | OD1 | ASP | A | 366 | 39.668 | 64.560 | 70.772 | 1.00 | 61.70 | A | O |
| ATOM | 2636 | OD2 | ASP | A | 366 | 39.640 | 64.065 | 68.641 | 1.00 | 60.29 | A | O |
| ATOM | 2637 | C | ASP | A | 366 | 42.324 | 61.154 | 71.352 | 1.00 | 51.89 | A | C |
| ATOM | 2638 | O | ASP | A | 366 | 41.653 | 60.400 | 72.064 | 1.00 | 48.46 | A | O |
| ATOM | 2639 | N | SER | A | 367 | 43.495 | 60.801 | 70.828 | 1.00 | 46.75 | A | N |
| ATOM | 2640 | CA | SER | A | 367 | 44.074 | 59.475 | 71.049 | 1.00 | 47.56 | A | C |
| ATOM | 2641 | CB | SER | A | 367 | 44.986 | 59.082 | 69.880 | 1.00 | 43.11 | A | C |
| ATOM | 2642 | OG | SER | A | 367 | 44.266 | 59.045 | 68.661 | 1.00 | 50.10 | A | O |
| ATOM | 2643 | C | SER | A | 367 | 44.870 | 59.437 | 72.344 | 1.00 | 49.63 | A | C |
| ATOM | 2644 | O | SER | A | 367 | 46.103 | 59.503 | 72.340 | 1.00 | 50.36 | A | O |
| ATOM | 2645 | N | VAL | A | 368 | 44.150 | 59.316 | 73.450 | 1.00 | 47.69 | A | N |
| ATOM | 2646 | CA | VAL | A | 368 | 44.763 | 59.287 | 74.765 | 1.00 | 48.39 | A | C |
| ATOM | 2647 | CB | VAL | A | 368 | 44.088 | 60.326 | 75.690 | 1.00 | 50.10 | A | C |
| ATOM | 2648 | CG1 | VAL | A | 368 | 44.793 | 60.365 | 77.035 | 1.00 | 47.89 | A | C |
| ATOM | 2649 | CG2 | VAL | A | 368 | 44.093 | 61.695 | 75.023 | 1.00 | 48.34 | A | C |
| ATOM | 2650 | C | VAL | A | 368 | 44.608 | 57.901 | 75.387 | 1.00 | 47.27 | A | C |
| ATOM | 2651 | O | VAL | A | 368 | 43.540 | 57.286 | 75.311 | 1.00 | 49.18 | A | O |
| ATOM | 2652 | N | GLU | A | 369 | 45.672 | 57.417 | 76.007 | 1.00 | 44.71 | A | N |
| ATOM | 2653 | CA | GLU | A | 369 | 45.655 | 56.110 | 76.645 | 1.00 | 44.66 | A | C |
| ATOM | 2654 | CB | GLU | A | 369 | 46.904 | 55.922 | 77.504 | 1.00 | 45.13 | A | C |
| ATOM | 2655 | CG | GLU | A | 369 | 48.202 | 55.867 | 76.756 | 1.00 | 42.79 | A | C |
| ATOM | 2656 | CD | GLU | A | 369 | 49.345 | 55.490 | 77.677 | 1.00 | 51.65 | A | C |
| ATOM | 2657 | OE1 | GLU | A | 369 | 49.676 | 56.303 | 78.574 | 1.00 | 50.70 | A | O |
| ATOM | 2658 | OE2 | GLU | A | 369 | 49.899 | 54.376 | 77.517 | 1.00 | 48.85 | A | O |
| ATOM | 2659 | C | GLU | A | 369 | 44.436 | 55.873 | 77.529 | 1.00 | 44.26 | A | C |
| ATOM | 2660 | O | GLU | A | 369 | 44.042 | 56.734 | 78.314 | 1.00 | 42.52 | A | O |
| ATOM | 2661 | N | ARG | A | 370 | 43.864 | 54.683 | 77.394 | 1.00 | 43.60 | A | N |
| ATOM | 2662 | CA | ARG | A | 370 | 42.712 | 54.240 | 78.163 | 1.00 | 47.16 | A | C |
| ATOM | 2663 | CB | ARG | A | 370 | 41.761 | 53.489 | 77.230 | 1.00 | 52.77 | A | C |
| ATOM | 2664 | CG | ARG | A | 370 | 40.701 | 52.643 | 77.893 | 1.00 | 59.69 | A | C |
| ATOM | 2665 | CD | ARG | A | 370 | 39.663 | 53.490 | 78.565 | 1.00 | 69.65 | A | C |
| ATOM | 2666 | NE | ARG | A | 370 | 38.441 | 52.734 | 78.815 | 1.00 | 79.66 | A | N |
| ATOM | 2667 | CZ | ARG | A | 370 | 37.450 | 53.159 | 79.594 | 1.00 | 85.53 | A | C |
| ATOM | 2668 | NH1 | ARG | A | 370 | 37.540 | 54.337 | 80.206 | 1.00 | 85.97 | A | N |
| ATOM | 2669 | NH2 | ARG | A | 370 | 36.368 | 52.406 | 79.760 | 1.00 | 86.59 | A | N |
| ATOM | 2670 | C | ARG | A | 370 | 43.335 | 53.293 | 79.197 | 1.00 | 49.24 | A | C |
| ATOM | 2671 | O | ARG | A | 370 | 43.651 | 52.144 | 78.882 | 1.00 | 46.55 | A | O |
| ATOM | 2672 | N | LEU | A | 371 | 43.517 | 53.786 | 80.422 | 1.00 | 51.20 | A | N |
| ATOM | 2673 | CA | LEU | A | 371 | 44.152 | 53.016 | 81.491 | 1.00 | 51.92 | A | C |
| ATOM | 2674 | CB | LEU | A | 371 | 45.327 | 53.805 | 82.063 | 1.00 | 45.34 | A | C |
| ATOM | 2675 | CG | LEU | A | 371 | 46.462 | 54.225 | 81.149 | 1.00 | 44.05 | A | C |
| ATOM | 2676 | CD1 | LEU | A | 371 | 47.469 | 55.038 | 81.941 | 1.00 | 44.55 | A | C |
| ATOM | 2677 | CD2 | LEU | A | 371 | 47.112 | 52.984 | 80.559 | 1.00 | 46.11 | A | C |
| ATOM | 2678 | C | LEU | A | 371 | 43.271 | 52.625 | 82.666 | 1.00 | 56.07 | A | C |
| ATOM | 2679 | O | LEU | A | 371 | 42.470 | 53.416 | 83.140 | 1.00 | 59.39 | A | O |
| ATOM | 2680 | N | PRO | A | 372 | 43.428 | 51.394 | 83.169 | 1.00 | 59.81 | A | N |
| ATOM | 2681 | CD | PRO | A | 372 | 44.206 | 50.267 | 82.625 | 1.00 | 58.00 | A | C |
| ATOM | 2682 | CA | PRO | A | 372 | 42.621 | 50.963 | 84.312 | 1.00 | 64.28 | A | C |
| ATOM | 2683 | CB | PRO | A | 372 | 42.603 | 49.452 | 84.158 | 1.00 | 60.72 | A | C |
| ATOM | 2684 | CG | PRO | A | 372 | 43.989 | 49.181 | 83.681 | 1.00 | 59.74 | A | C |
| ATOM | 2685 | C | PRO | A | 372 | 43.358 | 51.400 | 85.582 | 1.00 | 71.45 | A | C |
| ATOM | 2686 | O | PRO | A | 372 | 44.588 | 51.320 | 85.649 | 1.00 | 72.69 | A | O |
| ATOM | 2687 | N | VAL | A | 373 | 42.619 | 51.858 | 86.586 | 1.00 | 79.52 | A | N |
| ATOM | 2688 | CA | VAL | A | 373 | 43.233 | 52.304 | 87.834 | 1.00 | 87.31 | A | C |
| ATOM | 2689 | CB | VAL | A | 373 | 43.130 | 53.845 | 87.982 | 1.00 | 84.91 | A | C |
| ATOM | 2690 | CG1 | VAL | A | 373 | 43.572 | 54.281 | 89.375 | 1.00 | 83.39 | A | C |
| ATOM | 2691 | CG2 | VAL | A | 373 | 43.997 | 54.512 | 86.940 | 1.00 | 82.31 | A | C |
| ATOM | 2692 | C | VAL | A | 373 | 42.610 | 51.651 | 89.059 | 1.00 | 95.37 | A | C |
| ATOM | 2693 | O | VAL | A | 373 | 41.426 | 51.317 | 89.063 | 1.00 | 94.12 | A | O |
| ATOM | 2694 | N | TYR | A | 374 | 43.420 | 51.470 | 90.099 | 1.00 | 106.83 | A | N |
| ATOM | 2695 | CA | TYR | A | 374 | 42.947 | 50.868 | 91.339 | 1.00 | 119.05 | A | C |
| ATOM | 2696 | CB | TYR | A | 374 | 44.089 | 50.130 | 92.049 | 1.00 | 123.49 | A | C |
| ATOM | 2697 | CG | TYR | A | 374 | 43.693 | 49.597 | 93.408 | 1.00 | 129.33 | A | C |
| ATOM | 2698 | CD1 | TYR | A | 374 | 42.641 | 48.691 | 93.541 | 1.00 | 131.91 | A | C |
| ATOM | 2699 | CE1 | TYR | A | 374 | 42.241 | 48.226 | 94.791 | 1.00 | 134.37 | A | C |
| ATOM | 2700 | CD2 | TYR | A | 374 | 44.342 | 50.026 | 94.566 | 1.00 | 131.68 | A | C |
| ATOM | 2701 | CE2 | TYR | A | 374 | 43.950 | 49.568 | 95.824 | 1.00 | 134.23 | A | C |
| ATOM | 2702 | CZ | TYR | A | 374 | 42.897 | 48.669 | 95.928 | 1.00 | 135.04 | A | C |
| ATOM | 2703 | OH | TYR | A | 374 | 42.496 | 48.213 | 97.164 | 1.00 | 136.56 | A | O |
| ATOM | 2704 | C | TYR | A | 374 | 42.355 | 51.915 | 92.283 | 1.00 | 124.34 | A | C |
| ATOM | 2705 | O | TYR | A | 374 | 43.060 | 52.806 | 92.761 | 1.00 | 125.36 | A | O |
| ATOM | 2706 | N | ASN | A | 375 | 41.056 | 51.797 | 92.542 | 1.00 | 130.22 | A | N |
| ATOM | 2707 | CA | ASN | A | 375 | 40.346 | 52.710 | 93.437 | 1.00 | 135.75 | A | C |
| ATOM | 2708 | CB | ASN | A | 375 | 39.625 | 53.802 | 92.642 | 1.00 | 135.02 | A | C |
| ATOM | 2709 | CG | ASN | A | 375 | 40.571 | 54.662 | 91.839 | 1.00 | 135.13 | A | C |
| ATOM | 2710 | OD1 | ASN | A | 375 | 41.504 | 55.252 | 92.383 | 1.00 | 135.35 | A | O |
| ATOM | 2711 | ND2 | ASN | A | 375 | 40.333 | 54.746 | 90.535 | 1.00 | 134.74 | A | N |
| ATOM | 2712 | C | ASN | A | 375 | 39.310 | 51.914 | 94.218 | 1.00 | 139.47 | A | C |
| ATOM | 2713 | O | ASN | A | 375 | 39.227 | 50.692 | 94.085 | 1.00 | 141.24 | A | O |
| ATOM | 2714 | N | LYS | A | 376 | 38.526 | 52.606 | 95.039 | 1.00 | 142.47 | A | N |
| ATOM | 2715 | CA | LYS | A | 376 | 37.471 | 51.950 | 95.799 | 1.00 | 144.26 | A | C |
| ATOM | 2716 | CB | LYS | A | 376 | 36.900 | 52.903 | 96.857 | 1.00 | 141.45 | A | C |
| ATOM | 2717 | CG | LYS | A | 376 | 35.493 | 52.555 | 97.332 | 1.00 | 136.03 | A | C |
| ATOM | 2718 | CD | LYS | A | 376 | 35.417 | 51.160 | 97.931 | 1.00 | 130.81 | A | C |
| ATOM | 2719 | CE | LYS | A | 376 | 33.970 | 50.739 | 98.139 | 1.00 | 127.43 | A | C |
| ATOM | 2720 | NZ | LYS | A | 376 | 33.234 | 51.699 | 99.006 | 1.00 | 124.44 | A | N |
| ATOM | 2721 | C | LYS | A | 376 | 36.395 | 51.579 | 94.783 | 1.00 | 146.77 | A | C |
| ATOM | 2722 | O | LYS | A | 376 | 35.545 | 50.724 | 95.033 | 1.00 | 148.91 | A | O |
| ATOM | 2723 | N | SER | A | 377 | 36.457 | 52.235 | 93.626 | 1.00 | 147.73 | A | N |
| ATOM | 2724 | CA | SER | A | 377 | 35.516 | 52.004 | 92.534 | 1.00 | 147.25 | A | C |
| ATOM | 2725 | CB | SER | A | 377 | 35.683 | 53.082 | 91.463 | 1.00 | 147.55 | A | C |
| ATOM | 2726 | OG | SER | A | 377 | 34.889 | 52.802 | 90.324 | 1.00 | 146.68 | A | O |
| ATOM | 2727 | C | SER | A | 377 | 35.733 | 50.632 | 91.904 | 1.00 | 146.79 | A | C |
| ATOM | 2728 | O | SER | A | 377 | 34.790 | 49.859 | 91.733 | 1.00 | 147.01 | A | O |
| ATOM | 2729 | N | ALA | A | 378 | 36.983 | 50.340 | 91.556 | 1.00 | 145.47 | A | N |
| ATOM | 2730 | CA | ALA | A | 378 | 37.338 | 49.065 | 90.943 | 1.00 | 144.56 | A | C |
| ATOM | 2731 | CB | ALA | A | 378 | 38.794 | 49.099 | 90.487 | 1.00 | 141.88 | A | C |
| ATOM | 2732 | C | ALA | A | 378 | 37.120 | 47.896 | 91.902 | 1.00 | 145.38 | A | C |
| ATOM | 2733 | O | ALA | A | 378 | 36.839 | 46.776 | 91.472 | 1.00 | 144.44 | A | O |
| ATOM | 2734 | N | TRP | A | 379 | 37.243 | 48.165 | 93.199 | 1.00 | 147.92 | A | N |
| ATOM | 2735 | CA | TRP | A | 379 | 37.079 | 47.142 | 94.229 | 1.00 | 151.25 | A | C |
| ATOM | 2736 | CB | TRP | A | 379 | 37.317 | 47.750 | 95.616 | 1.00 | 156.85 | A | C |
| ATOM | 2737 | CG | TRP | A | 379 | 37.276 | 46.736 | 96.723 | 1.00 | 164.02 | A | C |
| ATOM | 2738 | CD2 | TRP | A | 379 | 36.137 | 46.383 | 97.521 | 1.00 | 167.57 | A | C |
| ATOM | 2739 | CE2 | TRP | A | 379 | 36.544 | 45.346 | 98.392 | 1.00 | 168.95 | A | C |
| ATOM | 2740 | CE3 | TRP | A | 379 | 34.814 | 46.844 | 97.586 | 1.00 | 169.11 | A | C |
| ATOM | 2741 | CD1 | TRP | A | 379 | 38.295 | 45.922 | 97.130 | 1.00 | 166.08 | A | C |
| ATOM | 2742 | NE1 | TRP | A | 379 | 37.862 | 45.084 | 98.132 | 1.00 | 168.40 | A | N |
| ATOM | 2743 | CZ2 | TRP | A | 379 | 35.670 | 44.758 | 99.315 | 1.00 | 170.17 | A | C |
| ATOM | 2744 | CZ3 | TRP | A | 379 | 33.946 | 46.258 | 98.504 | 1.00 | 170.25 | A | C |
| ATOM | 2745 | CH2 | TRP | A | 379 | 34.381 | 45.227 | 99.357 | 1.00 | 170.63 | A | C |
| ATOM | 2746 | C | TRP | A | 379 | 35.717 | 46.444 | 94.224 | 1.00 | 150.61 | A | C |
| ATOM | 2747 | O | TRP | A | 379 | 35.644 | 45.215 | 94.257 | 1.00 | 150.24 | A | O |
| ATOM | 2748 | N | ARG | A | 380 | 34.641 | 47.226 | 94.186 | 1.00 | 150.21 | A | N |
| ATOM | 2749 | CA | ARG | A | 380 | 33.291 | 46.670 | 94.212 | 1.00 | 150.17 | A | C |
| ATOM | 2750 | CB | ARG | A | 380 | 32.299 | 47.755 | 94.642 | 1.00 | 152.87 | A | C |
| ATOM | 2751 | CG | ARG | A | 380 | 32.505 | 49.105 | 93.980 | 1.00 | 157.37 | A | C |
| ATOM | 2752 | CD | ARG | A | 380 | 31.800 | 50.198 | 94.768 | 1.00 | 161.45 | A | C |
| ATOM | 2753 | NE | ARG | A | 380 | 32.018 | 51.525 | 94.198 | 1.00 | 165.00 | A | N |
| ATOM | 2754 | CZ | ARG | A | 380 | 31.662 | 52.660 | 94.791 | 1.00 | 166.90 | A | C |
| ATOM | 2755 | NH1 | ARG | A | 380 | 31.071 | 52.635 | 95.978 | 1.00 | 167.97 | A | N |
| ATOM | 2756 | NH2 | ARG | A | 380 | 31.898 | 53.824 | 94.199 | 1.00 | 167.99 | A | N |
| ATOM | 2757 | C | ARG | A | 380 | 32.793 | 45.970 | 92.946 | 1.00 | 148.71 | A | C |
| ATOM | 2758 | O | ARG | A | 380 | 31.665 | 45.478 | 92.914 | 1.00 | 147.97 | A | O |
| ATOM | 2759 | N | HIS | A | 381 | 33.621 | 45.919 | 91.905 | 1.00 | 147.67 | A | N |
| ATOM | 2760 | CA | HIS | A | 381 | 33.230 | 45.233 | 90.674 | 1.00 | 146.93 | A | C |
| ATOM | 2761 | CB | HIS | A | 381 | 34.038 | 45.742 | 89.476 | 1.00 | 145.78 | A | C |
| ATOM | 2762 | CG | HIS | A | 381 | 33.605 | 47.085 | 88.975 | 1.00 | 144.42 | A | C |
| ATOM | 2763 | CD2 | HIS | A | 381 | 33.050 | 47.458 | 87.797 | 1.00 | 144.04 | A | C |
| ATOM | 2764 | ND1 | HIS | A | 381 | 33.731 | 48.236 | 89.720 | 1.00 | 143.71 | A | N |
| ATOM | 2765 | CE1 | HIS | A | 381 | 33.274 | 49.262 | 89.024 | 1.00 | 143.37 | A | C |
| ATOM | 2766 | NE2 | HIS | A | 381 | 32.855 | 48.816 | 87.853 | 1.00 | 143.45 | A | N |
| ATOM | 2767 | C | HIS | A | 381 | 33.492 | 43.741 | 90.867 | 1.00 | 146.98 | A | C |
| ATOM | 2768 | O | HIS | A | 381 | 33.092 | 42.910 | 90.047 | 1.00 | 147.14 | A | O |
| ATOM | 2769 | N | TYR | A | 382 | 34.171 | 43.421 | 91.964 | 1.00 | 146.42 | A | N |
| ATOM | 2770 | CA | TYR | A | 382 | 34.511 | 42.046 | 92.310 | 1.00 | 145.88 | A | C |
| ATOM | 2771 | CB | TYR | A | 382 | 35.975 | 41.963 | 92.738 | 1.00 | 138.57 | A | C |
| ATOM | 2772 | CG | TYR | A | 382 | 36.959 | 42.298 | 91.646 | 1.00 | 130.90 | A | C |
| ATOM | 2773 | CD1 | TYR | A | 382 | 37.156 | 41.434 | 90.568 | 1.00 | 127.20 | A | C |
| ATOM | 2774 | CE1 | TYR | A | 382 | 38.076 | 41.732 | 89.566 | 1.00 | 123.80 | A | C |
| ATOM | 2775 | CD2 | TYR | A | 382 | 37.705 | 43.475 | 91.693 | 1.00 | 127.43 | A | C |
| ATOM | 2776 | CE2 | TYR | A | 382 | 38.627 | 43.783 | 90.697 | 1.00 | 123.96 | A | C |
| ATOM | 2777 | CZ | TYR | A | 382 | 38.807 | 42.909 | 89.639 | 1.00 | 122.89 | A | C |
| ATOM | 2778 | OH | TYR | A | 382 | 39.718 | 43.212 | 88.655 | 1.00 | 120.13 | A | O |
| ATOM | 2779 | C | TYR | A | 382 | 33.629 | 41.541 | 93.444 | 1.00 | 149.34 | A | C |
| ATOM | 2780 | O | TYR | A | 382 | 33.855 | 40.456 | 93.981 | 1.00 | 150.45 | A | O |
| ATOM | 2781 | N | GLN | A | 383 | 32.631 | 42.337 | 93.813 | 1.00 | 152.89 | A | N |
| ATOM | 2782 | CA | GLN | A | 383 | 31.722 | 41.959 | 94.886 | 1.00 | 155.46 | A | C |
| ATOM | 2783 | CB | GLN | A | 383 | 31.433 | 43.161 | 95.792 | 1.00 | 156.69 | A | C |
| ATOM | 2784 | CG | GLN | A | 383 | 32.672 | 43.866 | 96.345 | 1.00 | 157.01 | A | C |
| ATOM | 2785 | CD | GLN | A | 383 | 33.708 | 42.905 | 96.905 | 1.00 | 156.74 | A | C |
| ATOM | 2786 | OE1 | GLN | A | 383 | 34.681 | 42.562 | 96.232 | 1.00 | 156.34 | A | O |
| ATOM | 2787 | NE2 | GLN | A | 383 | 33.497 | 42.459 | 98.139 | 1.00 | 156.08 | A | N |
| ATOM | 2788 | C | GLN | A | 383 | 30.415 | 41.418 | 94.314 | 1.00 | 155.88 | A | C |
| ATOM | 2789 | O | GLN | A | 383 | 29.376 | 41.455 | 94.976 | 1.00 | 157.44 | A | O |
| ATOM | 2790 | N | THR | A | 384 | 30.474 | 40.919 | 93.081 | 1.00 | 154.83 | A | N |
| ATOM | 2791 | CA | THR | A | 384 | 29.300 | 40.363 | 92.411 | 1.00 | 152.44 | A | C |
| ATOM | 2792 | CB | THR | A | 384 | 29.618 | 39.978 | 90.942 | 1.00 | 150.39 | A | C |
| ATOM | 2793 | OG1 | THR | A | 384 | 30.111 | 41.126 | 90.239 | 1.00 | 147.26 | A | O |
| ATOM | 2794 | CG2 | THR | A | 384 | 28.367 | 39.466 | 90.241 | 1.00 | 147.32 | A | C |
| ATOM | 2795 | C | THR | A | 384 | 28.807 | 39.121 | 93.155 | 1.00 | 152.76 | A | C |
| ATOM | 2796 | O | THR | A | 384 | 29.463 | 38.078 | 93.147 | 1.00 | 153.24 | A | O |
| ATOM | 2797 | N | ILE | A | 385 | 27.648 | 39.248 | 93.797 | 1.00 | 151.88 | A | N |
| ATOM | 2798 | CA | ILE | A | 385 | 27.047 | 38.156 | 94.563 | 1.00 | 150.44 | A | C |
| ATOM | 2799 | CB | ILE | A | 385 | 26.704 | 38.626 | 96.001 | 1.00 | 146.21 | A | C |
| ATOM | 2800 | CG2 | ILE | A | 385 | 26.328 | 37.432 | 96.870 | 1.00 | 144.46 | A | C |
| ATOM | 2801 | CG1 | ILE | A | 385 | 27.907 | 39.349 | 96.613 | 1.00 | 142.13 | A | C |
| ATOM | 2802 | CD1 | ILE | A | 385 | 27.613 | 40.031 | 97.934 | 1.00 | 136.32 | A | C |
| ATOM | 2803 | C | ILE | A | 385 | 25.762 | 37.695 | 93.867 | 1.00 | 152.77 | A | C |
| ATOM | 2804 | O | ILE | A | 385 | 24.778 | 37.342 | 94.520 | 1.00 | 153.45 | A | O |
| ATOM | 2805 | N | ALA | A | 386 | 25.789 | 37.694 | 92.538 | 1.00 | 154.68 | A | N |
| ATOM | 2806 | CA | ALA | A | 386 | 24.637 | 37.306 | 91.729 | 1.00 | 156.79 | A | C |
| ATOM | 2807 | CB | ALA | A | 386 | 25.017 | 37.325 | 90.252 | 1.00 | 156.03 | A | C |
| ATOM | 2808 | C | ALA | A | 386 | 24.001 | 35.961 | 92.085 | 1.00 | 158.47 | A | C |
| ATOM | 2809 | O | ALA | A | 386 | 24.687 | 34.959 | 92.300 | 1.00 | 158.74 | A | O |
| ATOM | 2810 | N | ALA | A | 387 | 22.672 | 35.966 | 92.131 | 1.00 | 160.41 | A | N |
| ATOM | 2811 | CA | ALA | A | 387 | 21.858 | 34.794 | 92.442 | 1.00 | 161.58 | A | C |
| ATOM | 2812 | CB | ALA | A | 387 | 22.128 | 34.319 | 93.867 | 1.00 | 162.31 | A | C |
| ATOM | 2813 | C | ALA | A | 387 | 20.405 | 35.242 | 92.296 | 1.00 | 161.68 | A | C |
| ATOM | 2814 | O | ALA | A | 387 | 19.845 | 35.858 | 93.206 | 1.00 | 162.85 | A | O |
| ATOM | 2815 | N | ALA | A | 388 | 19.811 | 34.934 | 91.142 | 1.00 | 160.20 | A | N |
| ATOM | 2816 | CA | ALA | A | 388 | 18.436 | 35.319 | 90.820 | 1.00 | 157.50 | A | C |
| ATOM | 2817 | CB | ALA | A | 388 | 17.522 | 35.144 | 92.035 | 1.00 | 157.89 | A | C |
| ATOM | 2818 | C | ALA | A | 388 | 18.462 | 36.780 | 90.374 | 1.00 | 154.87 | A | C |
| ATOM | 2819 | O | ALA | A | 388 | 18.608 | 37.686 | 91.195 | 1.00 | 155.84 | A | O |
| ATOM | 2820 | N | GLY | A | 389 | 18.324 | 36.999 | 89.070 | 1.00 | 150.57 | A | N |
| ATOM | 2821 | CA | GLY | A | 389 | 18.366 | 38.347 | 88.533 | 1.00 | 144.15 | A | C |
| ATOM | 2822 | C | GLY | A | 389 | 17.110 | 39.189 | 88.665 | 1.00 | 139.15 | A | C |
| ATOM | 2823 | O | GLY | A | 389 | 16.177 | 38.850 | 89.393 | 1.00 | 139.50 | A | O |
| ATOM | 2824 | N | ASP | A | 390 | 17.104 | 40.305 | 87.943 | 1.00 | 133.13 | A | N |
| ATOM | 2825 | CA | ASP | A | 390 | 15.989 | 41.242 | 87.941 | 1.00 | 125.99 | A | C |
| ATOM | 2826 | CB | ASP | A | 390 | 16.515 | 42.680 | 87.820 | 1.00 | 127.73 | A | C |
| ATOM | 2827 | CG | ASP | A | 390 | 17.833 | 42.769 | 87.053 | 1.00 | 128.94 | A | C |
| ATOM | 2828 | OD1 | ASP | A | 390 | 18.209 | 41.794 | 86.371 | 1.00 | 129.88 | A | O |
| ATOM | 2829 | OD2 | ASP | A | 390 | 18.496 | 43.826 | 87.127 | 1.00 | 130.22 | A | O |
| ATOM | 2830 | C | ASP | A | 390 | 14.983 | 40.960 | 86.825 | 1.00 | 120.80 | A | C |
| ATOM | 2831 | O | ASP | A | 390 | 13.938 | 41.603 | 86.750 | 1.00 | 118.50 | A | O |
| ATOM | 2832 | N | TRP | A | 391 | 15.300 | 40.001 | 85.960 | 1.00 | 115.88 | A | N |
| ATOM | 2833 | CA | TRP | A | 391 | 14.410 | 39.646 | 84.857 | 1.00 | 112.78 | A | C |
| ATOM | 2834 | CB | TRP | A | 391 | 15.105 | 39.853 | 83.501 | 1.00 | 110.00 | A | C |
| ATOM | 2835 | CG | TRP | A | 391 | 15.752 | 41.204 | 83.350 | 1.00 | 107.92 | A | C |
| ATOM | 2836 | CD2 | TRP | A | 391 | 15.238 | 42.331 | 82.620 | 1.00 | 106.74 | A | C |
| ATOM | 2837 | CE2 | TRP | A | 391 | 16.149 | 43.397 | 82.817 | 1.00 | 106.59 | A | C |
| ATOM | 2838 | CE3 | TRP | A | 391 | 14.104 | 42.539 | 81.828 | 1.00 | 106.08 | A | C |
| ATOM | 2839 | CD1 | TRP | A | 391 | 16.913 | 41.622 | 83.930 | 1.00 | 107.17 | A | C |
| ATOM | 2840 | NE1 | TRP | A | 391 | 17.156 | 42.941 | 83.615 | 1.00 | 107.08 | A | N |
| ATOM | 2841 | CZ2 | TRP | A | 391 | 15.949 | 44.660 | 82.243 | 1.00 | 105.57 | A | C |
| ATOM | 2842 | CZ3 | TRP | A | 391 | 13.910 | 43.798 | 81.259 | 1.00 | 105.12 | A | C |
| ATOM | 2843 | CH2 | TRP | A | 391 | 14.831 | 44.841 | 81.471 | 1.00 | 104.94 | A | C |
| ATOM | 2844 | C | TRP | A | 391 | 13.976 | 38.192 | 84.990 | 1.00 | 112.66 | A | C |
| ATOM | 2845 | O | TRP | A | 391 | 14.693 | 37.374 | 85.565 | 1.00 | 112.24 | A | O |
| ATOM | 2846 | N | CYS | A | 392 | 12.796 | 37.878 | 84.461 | 1.00 | 113.88 | A | N |
| ATOM | 2847 | CA | CYS | A | 392 | 12.268 | 36.519 | 84.512 | 1.00 | 115.43 | A | C |
| ATOM | 2848 | CB | CYS | A | 392 | 10.990 | 36.463 | 85.356 | 1.00 | 114.72 | A | C |
| ATOM | 2849 | SG | CYS | A | 392 | 9.530 | 37.189 | 84.569 | 1.00 | 112.03 | A | S |
| ATOM | 2850 | C | CYS | A | 392 | 11.966 | 36.023 | 83.102 | 1.00 | 116.83 | A | C |
| ATOM | 2851 | O | CYS | A | 392 | 12.438 | 36.665 | 82.137 | 1.00 | 117.60 | A | O |
| ATOM | 2852 | OXT | CYS | A | 392 | 11.268 | 34.994 | 82.980 | 1.00 | 118.89 | A | O |
| TER | 2853 | | CYS | A | 392 | | | | | | A | |
| ATOM | 2854 | O | HOH | | 1 | 46.045 | 50.531 | 69.665 | 1.00 | 38.41 | | O |
| ATOM | 2855 | O | HOH | | 2 | 39.667 | 62.290 | 66.990 | 1.00 | 41.90 | | O |
| ATOM | 2856 | O | HOH | | 3 | 43.631 | 57.024 | 67.267 | 1.00 | 33.28 | | O |
| ATOM | 2857 | O | HOH | | 4 | 42.793 | 52.925 | 69.457 | 1.00 | 36.48 | | O |
| ATOM | 2858 | O | HOH | | 5 | 52.619 | 40.936 | 81.111 | 1.00 | 39.98 | | O |
| ATOM | 2859 | O | HOH | | 6 | 28.013 | 51.288 | 67.478 | 1.00 | 35.15 | | O |
| ATOM | 2860 | O | HOH | | 7 | 34.290 | 40.429 | 58.791 | 1.00 | 34.09 | | O |
| ATOM | 2861 | O | HOH | | 8 | 30.023 | 43.753 | 69.389 | 1.00 | 44.58 | | O |
| ATOM | 2862 | O | HOH | | 9 | 28.623 | 53.425 | 65.781 | 1.00 | 35.67 | | O |
| ATOM | 2863 | O | HOH | | 10 | 35.782 | 39.386 | 60.658 | 1.00 | 34.01 | | O |
| ATOM | 2864 | O | HOH | | 12 | 30.205 | 55.579 | 64.721 | 1.00 | 35.76 | | O |
| ATOM | 2865 | O | HOH | | 13 | 32.371 | 56.301 | 63.279 | 1.00 | 32.32 | | O |
| ATOM | 2866 | O | HOH | | 14 | 33.307 | 36.935 | 52.264 | 1.00 | 43.43 | | O |
| ATOM | 2867 | O | HOH | | 15 | 32.248 | 63.710 | 59.086 | 1.00 | 45.23 | | O |
| ATOM | 2868 | O | HOH | | 16 | 34.528 | 57.426 | 60.436 | 1.00 | 43.68 | | O |
| ATOM | 2869 | O | HOH | | 17 | 43.112 | 31.178 | 58.460 | 1.00 | 58.78 | | O |
| ATOM | 2870 | O | HOH | | 18 | 36.856 | 45.296 | 76.171 | 1.00 | 42.15 | | O |
| ATOM | 2871 | O | HOH | | 19 | 51.362 | 44.879 | 73.271 | 1.00 | 37.61 | | O |
| ATOM | 2872 | O | HOH | | 20 | 26.263 | 34.072 | 52.135 | 1.00 | 45.36 | | O |
| ATOM | 2873 | O | HOH | | 21 | 43.079 | 30.477 | 88.444 | 1.00 | 52.81 | | O |
| ATOM | 2874 | O | HOH | | 22 | 48.710 | 49.126 | 85.117 | 1.00 | 35.64 | | O |
| ATOM | 2875 | O | HOH | | 23 | 33.386 | 59.298 | 68.214 | 1.00 | 49.54 | | O |
| ATOM | 2876 | O | HOH | | 24 | 30.205 | 35.437 | 58.638 | 1.00 | 40.11 | | O |
| ATOM | 2877 | O | HOH | | 25 | 48.590 | 41.521 | 74.546 | 1.00 | 42.95 | | O |
| ATOM | 2878 | O | HOH | | 26 | 42.279 | 33.349 | 85.939 | 1.00 | 60.78 | | O |
| ATOM | 2879 | O | HOH | | 27 | 32.470 | 44.881 | 70.956 | 1.00 | 40.55 | | O |
| ATOM | 2880 | O | HOH | | 28 | 66.606 | 43.653 | 102.619 | 1.00 | 51.18 | | O |
| ATOM | 2881 | O | HOH | | 29 | 33.211 | 35.962 | 63.289 | 1.00 | 60.57 | | O |
| ATOM | 2882 | O | HOH | | 30 | 35.533 | 38.250 | 63.282 | 1.00 | 37.16 | | O |
| ATOM | 2883 | O | HOH | | 31 | 60.490 | 53.175 | 97.819 | 1.00 | 53.32 | | O |
| ATOM | 2884 | O | HOH | | 32 | 45.692 | 59.725 | 66.009 | 1.00 | 44.33 | | O |
| ATOM | 2885 | O | HOH | | 33 | 39.248 | 64.194 | 60.567 | 1.00 | 45.91 | | O |
| ATOM | 2886 | O | HOH | | 34 | 46.378 | 63.946 | 63.263 | 1.00 | 50.03 | | O |
| ATOM | 2887 | O | HOH | | 35 | 45.377 | 54.205 | 92.502 | 1.00 | 51.46 | | O |
| ATOM | 2888 | O | HOH | | 36 | 35.461 | 35.643 | 80.450 | 1.00 | 50.42 | | O |
| ATOM | 2889 | O | HOH | | 37 | 42.072 | 55.649 | 68.551 | 1.00 | 35.42 | | O |
| ATOM | 2890 | O | HOH | | 38 | 34.376 | 54.775 | 73.446 | 1.00 | 53.60 | | O |
| ATOM | 2891 | O | HOH | | 39 | 37.381 | 24.651 | 53.797 | 1.00 | 61.29 | | O |
| ATOM | 2892 | O | HOH | | 40 | 34.459 | 22.233 | 91.929 | 1.00 | 55.59 | | O |
| ATOM | 2893 | O | HOH | | 41 | 50.399 | 42.761 | 66.463 | 1.00 | 53.14 | | O |
| ATOM | 2894 | O | HOH | | 42 | 68.058 | 55.045 | 78.357 | 1.00 | 57.42 | | O |
| ATOM | 2895 | O | HOH | | 43 | 53.498 | 26.768 | 87.548 | 1.00 | 59.67 | | O |
| ATOM | 2896 | O | HOH | | 44 | 36.376 | 56.699 | 67.030 | 1.00 | 46.23 | | O |
| ATOM | 2897 | O | HOH | | 45 | 47.823 | 32.704 | 62.600 | 1.00 | 47.08 | | O |
| ATOM | 2898 | O | HOH | | 46 | 45.846 | 60.702 | 63.494 | 1.00 | 50.79 | | O |
| ATOM | 2899 | O | HOH | | 47 | 26.637 | 30.535 | 54.011 | 1.00 | 49.60 | | O |
| ATOM | 2900 | O | HOH | | 48 | 42.846 | 63.825 | 54.356 | 1.00 | 53.17 | | O |
| ATOM | 2901 | O | HOH | | 49 | 47.462 | 27.216 | 57.653 | 1.00 | 54.92 | | O |
| ATOM | 2902 | O | HOH | | 50 | 43.766 | 21.750 | 78.844 | 1.00 | 64.57 | | O |
| ATOM | 2903 | O | HOH | | 51 | 39.389 | 31.266 | 62.534 | 1.00 | 52.98 | | O |
| ATOM | 2904 | O | HOH | | 52 | 36.592 | 49.901 | 44.826 | 1.00 | 58.18 | | O |
| ATOM | 2905 | O | HOH | | 53 | 31.902 | 41.659 | 75.938 | 1.00 | 45.45 | | O |
| ATOM | 2906 | O | HOH | | 54 | 48.458 | 61.551 | 59.682 | 1.00 | 48.87 | | O |
| ATOM | 2907 | O | HOH | | 55 | 42.691 | 57.087 | 47.735 | 1.00 | 55.70 | | O |
| ATOM | 2908 | O | HOH | | 56 | 32.458 | 31.403 | 65.724 | 1.00 | 57.07 | | O |
| ATOM | 2909 | O | HOH | | 57 | 39.167 | 35.127 | 69.239 | 1.00 | 52.27 | | O |
| ATOM | 2910 | O | HOH | | 58 | 36.439 | 18.886 | 86.489 | 1.00 | 46.29 | | O |
| ATOM | 2911 | O | HOH | | 59 | 46.027 | 49.320 | 87.043 | 1.00 | 53.78 | | O |
| ATOM | 2912 | O | HOH | | 60 | 43.042 | 51.957 | 48.671 | 1.00 | 55.38 | | O |
| ATOM | 2913 | O | HOH | | 61 | 47.502 | 70.119 | 72.515 | 1.00 | 57.94 | | O |
| ATOM | 2914 | O | HOH | | 62 | 27.213 | 29.531 | 65.058 | 1.00 | 52.78 | | O |
| ATOM | 2915 | O | HOH | | 63 | 38.749 | 65.009 | 57.810 | 1.00 | 39.08 | | O |
| ATOM | 2916 | O | HOH | | 64 | 32.347 | 47.069 | 75.177 | 1.00 | 49.61 | | O |
| ATOM | 2917 | O | HOH | | 65 | 42.474 | 40.872 | 45.945 | 1.00 | 46.33 | | O |
| ATOM | 2918 | O | HOH | | 66 | 63.856 | 36.423 | 85.366 | 1.00 | 59.18 | | O |
| ATOM | 2919 | O | HOH | | 67 | 32.306 | 65.788 | 48.359 | 1.00 | 59.32 | | O |
| ATOM | 2920 | O | HOH | | 68 | 45.389 | 37.929 | 69.321 | 1.00 | 57.36 | | O |
| ATOM | 2921 | O | HOH | | 69 | 59.727 | 38.378 | 102.834 | 1.00 | 64.04 | | O |
| ATOM | 2922 | O | HOH | | 70 | 68.473 | 41.131 | 85.121 | 1.00 | 59.92 | | O |
| ATOM | 2923 | O | HOH | | 71 | 32.043 | 64.609 | 65.283 | 1.00 | 56.04 | | O |
| ATOM | 2924 | O | HOH | | 72 | 44.041 | 54.805 | 53.898 | 1.00 | 49.81 | | O |
| ATOM | 2925 | O | HOH | | 73 | 27.299 | 33.776 | 48.783 | 1.00 | 50.14 | | O |
| ATOM | 2926 | O | HOH | | 74 | 49.495 | 60.478 | 90.786 | 1.00 | 58.24 | | O |
| ATOM | 2927 | O | HOH | | 75 | 40.107 | 64.850 | 55.562 | 1.00 | 53.62 | | O |
| ATOM | 2928 | O | HOH | | 76 | 39.352 | 23.199 | 96.625 | 1.00 | 55.38 | | O |
| ATOM | 2929 | O | HOH | | 77 | 38.884 | 43.024 | 43.914 | 1.00 | 58.17 | | O |
| ATOM | 2930 | O | HOH | | 78 | 44.105 | 62.146 | 55.658 | 1.00 | 46.57 | | O |
| ATOM | 2931 | O | HOH | | 79 | 42.483 | 46.088 | 46.267 | 1.00 | 62.24 | | O |
| ATOM | 2932 | O | HOH | | 80 | 62.665 | 42.966 | 98.431 | 1.00 | 59.23 | | O |
| ATOM | 2933 | O | HOH | | 82 | 41.989 | 19.587 | 58.696 | 1.00 | 63.23 | | O |
| ATOM | 2934 | O | HOH | | 83 | 26.171 | 29.870 | 48.439 | 1.00 | 54.59 | | O |
| ATOM | 2935 | O | HOH | | 84 | 52.109 | 44.628 | 69.353 | 1.00 | 62.40 | | O |
| ATOM | 2936 | O | HOH | | 85 | 31.677 | 38.958 | 76.043 | 1.00 | 48.97 | | O |
| ATOM | 2937 | O | HOH | | 86 | 31.152 | 34.283 | 60.805 | 1.00 | 49.92 | | O |
| ATOM | 2938 | O | HOH | | 87 | 38.130 | 22.051 | 94.195 | 1.00 | 53.98 | | O |
| ATOM | 2939 | O | HOH | | 88 | 36.718 | 63.806 | 56.487 | 1.00 | 53.98 | | O |
| ATOM | 2940 | O | HOH | | 89 | 51.186 | 63.672 | 60.929 | 1.00 | 59.60 | | O |
| ATOM | 2941 | O | HOH | | 90 | 61.973 | 50.097 | 83.756 | 1.00 | 60.05 | | O |
| ATOM | 2942 | O | HOH | | 91 | 36.000 | 33.109 | 65.377 | 1.00 | 60.08 | | O |
| ATOM | 2943 | O | HOH | | 93 | 33.836 | 29.963 | 61.031 | 1.00 | 54.41 | | O |
| ATOM | 2944 | O | HOH | | 94 | 42.594 | 20.269 | 52.868 | 1.00 | 62.14 | | O |
| ATOM | 2945 | O | HOH | | 95 | 36.178 | 40.290 | 43.939 | 1.00 | 60.02 | | O |
| ATOM | 2946 | O | HOH | | 96 | 63.643 | 38.842 | 102.924 | 1.00 | 56.36 | | O |
| ATOM | 2947 | O | HOH | | 97 | 55.080 | 43.689 | 79.755 | 1.00 | 48.62 | | O |
| ATOM | 2948 | O | HOH | | 98 | 35.619 | 67.420 | 59.401 | 1.00 | 52.00 | | O |
| ATOM | 2949 | O | HOH | | 99 | 36.047 | 53.218 | 73.720 | 1.00 | 52.47 | | O |
| ATOM | 2950 | O | HOH | | 100 | 34.197 | 36.988 | 61.153 | 1.00 | 54.58 | | O |
| ATOM | 2951 | O | HOH | | 101 | 27.352 | 32.230 | 52.792 | 1.00 | 48.46 | | O |
| ATOM | 2952 | O | HOH | | 102 | 32.423 | 47.751 | 72.521 | 1.00 | 52.08 | | O |
| ATOM | 2953 | O | HOH | | 103 | 50.322 | 42.976 | 74.328 | 1.00 | 54.13 | | O |
| ATOM | 2954 | O | HOH | | 104 | 49.280 | 44.253 | 83.652 | 1.00 | 52.19 | | O |
| ATOM | 2955 | O | HOH | | 105 | 33.927 | 65.106 | 57.780 | 1.00 | 54.72 | | O |
| ATOM | 2956 | O | HOH | | 106 | 28.690 | 51.763 | 69.879 | 1.00 | 51.32 | | O |
| ATOM | 2957 | O | HOH | | 107 | 32.623 | 66.772 | 56.099 | 1.00 | 50.04 | | O |
| ATOM | 2958 | O | HOH | | 108 | 57.922 | 40.046 | 102.281 | 1.00 | 62.01 | | O |
| ATOM | 2959 | O | HOH | | 109 | 37.635 | 42.878 | 75.373 | 1.00 | 50.97 | | O |
| ATOM | 2960 | O | HOH | | 110 | 64.234 | 43.294 | 101.680 | 1.00 | 58.09 | | O |
| ATOM | 2961 | O | HOH | | 111 | 51.523 | 47.534 | 73.535 | 1.00 | 50.86 | | O |
| TER | 2962 | | HOH | | 111 | | | | | | | |
| ATOM | 2963 | N1 | HET | | 1 | 57.056 | 60.199 | 76.835 | 1.00 | 65.84 | | N |
| ATOM | 2964 | C1 | HET | | 1 | 57.675 | 59.299 | 75.915 | 1.00 | 67.95 | | C |
| ATOM | 2965 | C2 | HET | | 1 | 56.396 | 61.342 | 76.145 | 1.00 | 64.07 | | C |
| ATOM | 2966 | C3 | HET | | 1 | 56.953 | 60.118 | 78.330 | 1.00 | 65.40 | | C |
| ATOM | 2967 | C4 | HET | | 1 | 58.408 | 57.990 | 76.452 | 1.00 | 72.24 | | C |
| ATOM | 2968 | O1 | HET | | 1 | 57.642 | 59.525 | 74.706 | 1.00 | 67.36 | | O |
| ATOM | 2969 | C5 | HET | | 1 | 56.831 | 62.639 | 76.844 | 1.00 | 63.87 | | C |
| ATOM | 2970 | C6 | HET | | 1 | 54.858 | 61.116 | 76.131 | 1.00 | 60.36 | | C |
| ATOM | 2971 | C7 | HET | | 1 | 57.280 | 61.446 | 79.047 | 1.00 | 66.14 | | C |
| ATOM | 2972 | C8 | HET | | 1 | 57.433 | 56.836 | 76.971 | 1.00 | 72.70 | | C |
| ATOM | 2973 | C9 | HET | | 1 | 59.139 | 57.418 | 75.205 | 1.00 | 73.21 | | C |
| ATOM | 2974 | O2 | HET | | 1 | 59.387 | 58.292 | 77.328 | 1.00 | 71.92 | | O |
| ATOM | 2975 | N2 | HET | | 1 | 56.561 | 62.528 | 78.308 | 1.00 | 66.48 | | N |
| ATOM | 2976 | C10 | HET | | 1 | 58.803 | 61.753 | 79.122 | 1.00 | 64.53 | | C |
| ATOM | 2977 | F1 | HET | | 1 | 56.740 | 56.339 | 75.981 | 1.00 | 74.89 | | F |
| ATOM | 2978 | F2 | HET | | 1 | 58.127 | 55.828 | 77.465 | 1.00 | 73.26 | | F |
| ATOM | 2979 | F3 | HET | | 1 | 56.570 | 57.238 | 77.895 | 1.00 | 73.75 | | F |
| ATOM | 2980 | C11 | HET | | 1 | 55.681 | 63.433 | 78.898 | 1.00 | 67.33 | | C |
| ATOM | 2981 | C12 | HET | | 1 | 55.374 | 63.394 | 80.337 | 1.00 | 67.77 | | C |
| ATOM | 2982 | O3 | HET | | 1 | 55.149 | 64.288 | 78.198 | 1.00 | 69.37 | | O |
| ATOM | 2983 | C13 | HET | | 1 | 56.336 | 63.896 | 81.293 | 1.00 | 69.28 | | C |
| ATOM | 2984 | C14 | HET | | 1 | 54.100 | 62.904 | 80.748 | 1.00 | 68.55 | | C |
| ATOM | 2985 | C15 | HET | | 1 | 55.998 | 63.906 | 82.691 | 1.00 | 69.62 | | C |
| ATOM | 2986 | C16 | HET | | 1 | 53.762 | 62.913 | 82.126 | 1.00 | 72.23 | | C |
| ATOM | 2987 | C17 | HET | | 1 | 54.711 | 63.409 | 83.085 | 1.00 | 73.27 | | C |
| ATOM | 2988 | C18 | HET | | 1 | 54.357 | 63.395 | 84.469 | 1.00 | 76.77 | | C |
| ATOM | 2989 | N3 | HET | | 1 | 54.062 | 63.369 | 85.588 | 1.00 | 83.47 | | N |
| TER | 2990 | | HET | | 1 | | | | | | | |
| END | | | | | | | | | | | | |

TABLE 5
Atomic coordinates of PDHK-2* with Compound 1 and ATP

REMARK coordinates from restrained individual B-factor refinement
REMARK refinement resolution: 30 - 2.4 A

| | | |
|---|---|---|
| REMARK starting | r = 0.2381 | free_r = 0.2465 |
| REMARK final | r = 0.2380 | free_r = 0.2465 |

| | | |
|---|---|---|
| REMARK B rmsd for bonded mainchain atoms = | 1.220 | target = 1.5 |
| REMARK B rmsd for bonded sidechain atoms = | 1.393 | target = 2.0 |
| REMARK B rmsd for angle mainchain atoms = | 2.209 | target = 2.0 |
| REMARK B rmsd for angle sidechain atoms = | 2.217 | target = 2.5 |

REMARK wa = 1.92809
REMARK rweight = 0.194546
REMARK target = mlf steps = 10
REMARK sg = P6(4) a = 108.703 b = 108.703 c = 83.855 alpha = 90 beta = 90 gamma = 120
REMARK parameter file 1 : MSI_CNX_TOPPAR:protein_rep.param
REMARK parameter file 2 : . . . /toppar/het.par
REMARK parameter file 3 : MSI_CNX_TOPPAR:water_rep.param
REMARK parameter file 4 : MSI_CNX_TOPPAR:ion.param
REMARK parameter file 5 : . . . /toppar/atp.par
REMARK molecular structure file: . . . /generate/generate.psf
REMARK input coordinates: . . . /generate/generate.pdb
REMARK reflection file = . . . /pdhk65.cv
REMARK ncs = none
REMARK B-correction resolution: 6.0 − 2.4
REMARK initial B-factor correction applied to fobs :

| | | | | | | |
|---|---|---|---|---|---|---|
| REMARK | B11 = | −4.976 | B22 = | −4.976 | B33 = | 9.953 |
| REMARK | B12 = | −5.980 | B13 = | 0.000 | B23 = | 0.000 |

REMARK B-factor correction applied to coordinate array B: 0.104
REMARK bulk solvent: (Mask) density level = 0.345542 e/A\hat{3}, B-factor = 46.9776 A\hat{2}
REMARK reflections with |Fobs|/sigma_F < 0.0 rejected
REMARK reflections with |Fobs| > 10000 * rms(Fobs) rejected

| | | |
|---|---|---|
| REMARK theoretical total number of ref1. in resol. range: | 22116 | (100.0%) |
| REMARK number of unobserved reflections (no entry or \|F\| = 0) : | 228 | (1.0%) |
| REMARK number of reflections rejected: | 0 | (0.0%) |
| REMARK total number of reflections used: | 21888 | (99.0%) |
| REMARK number of reflections in working set: | 20821 | (94.1%) |
| REMARK number of reflections in test set: | 1067 | (4.8%) |
| REMARK FILENAME = “bindividual.pdb” | | |
| REMARK DATE:Apr. 03, 2001 16:38:01 created by user: knoechel | | |
| REMARK Written by CNX VERSION:2000 | | |

| | | | | | | | | | | | |
|---|---|---|---|---|---|---|---|---|---|---|---|
| ATOM | 1 | C | GLY | A | 14 | 61.288 | 42.301 | 101.224 | 1.00 | 62.32 | C |
| ATOM | 2 | O | GLY | A | 14 | 60.553 | 42.392 | 102.207 | 1.00 | 62.01 | O |
| ATOM | 3 | N | GLY | A | 14 | 63.377 | 41.411 | 100.264 | 1.00 | 62.74 | N |
| ATOM | 4 | CA | GLY | A | 14 | 62.161 | 41.079 | 101.032 | 1.00 | 61.98 | C |
| ATOM | 5 | N | SER | A | 15 | 61.353 | 43.239 | 100.283 | 1.00 | 62.80 | N |
| ATOM | 6 | CA | SER | A | 15 | 60.551 | 44.461 | 100.381 | 1.00 | 64.07 | C |
| ATOM | 7 | CB | SER | A | 15 | 61.469 | 45.662 | 100.674 | 1.00 | 64.75 | C |
| ATOM | 8 | OG | SER | A | 15 | 62.479 | 45.816 | 99.685 | 1.00 | 67.36 | O |
| ATOM | 9 | C | SER | A | 15 | 59.641 | 44.776 | 99.180 | 1.00 | 63.50 | C |
| ATOM | 10 | O | SER | A | 15 | 59.251 | 45.926 | 98.979 | 1.00 | 63.30 | O |
| ATOM | 11 | N | ALA | A | 16 | 59.295 | 43.765 | 98.389 | 1.00 | 63.56 | N |
| ATOM | 12 | CA | ALA | A | 16 | 58.417 | 43.988 | 97.241 | 1.00 | 63.48 | C |
| ATOM | 13 | CB | ALA | A | 16 | 58.256 | 42.702 | 96.436 | 1.00 | 63.69 | C |
| ATOM | 14 | C | ALA | A | 16 | 57.049 | 44.493 | 97.703 | 1.00 | 63.73 | C |
| ATOM | 15 | O | ALA | A | 16 | 56.491 | 45.414 | 97.113 | 1.00 | 63.41 | O |
| ATOM | 16 | N | PRO | A | 17 | 56.489 | 43.891 | 98.767 | 1.00 | 63.72 | N |
| ATOM | 17 | CD | PRO | A | 17 | 56.937 | 42.685 | 99.484 | 1.00 | 63.39 | C |
| ATOM | 18 | CA | PRO | A | 17 | 55.180 | 44.328 | 99.263 | 1.00 | 64.13 | C |
| ATOM | 19 | CB | PRO | A | 17 | 54.926 | 43.388 | 100.439 | 1.00 | 63.56 | C |
| ATOM | 20 | CG | PRO | A | 17 | 55.642 | 42.149 | 100.037 | 1.00 | 62.97 | C |
| ATOM | 21 | C | PRO | A | 17 | 55.171 | 45.794 | 99.694 | 1.00 | 64.83 | C |
| ATOM | 22 | O | PRO | A | 17 | 54.119 | 46.441 | 99.712 | 1.00 | 64.94 | O |
| ATOM | 23 | N | LYS | A | 18 | 56.346 | 46.306 | 100.051 | 1.00 | 65.65 | N |
| ATOM | 24 | CA | LYS | A | 18 | 56.475 | 47.692 | 100.484 | 1.00 | 65.89 | C |
| ATOM | 25 | CB | LYS | A | 18 | 57.777 | 47.885 | 101.260 | 1.00 | 66.68 | C |
| ATOM | 26 | CG | LYS | A | 18 | 58.020 | 49.319 | 101.712 | 1.00 | 68.08 | C |
| ATOM | 27 | CD | LYS | A | 18 | 59.423 | 49.502 | 102.263 | 1.00 | 69.34 | C |
| ATOM | 28 | CE | LYS | A | 18 | 59.732 | 50.976 | 102.482 | 1.00 | 70.28 | C |
| ATOM | 29 | NZ | LYS | A | 18 | 61.191 | 51.214 | 102.649 | 1.00 | 70.11 | N |
| ATOM | 30 | C | LYS | A | 18 | 56.464 | 48.604 | 99.262 | 1.00 | 65.51 | C |
| ATOM | 31 | O | LYS | A | 18 | 55.720 | 49.581 | 99.207 | 1.00 | 64.93 | O |
| ATOM | 32 | N | TYR | A | 19 | 57.302 | 48.275 | 98.288 | 1.00 | 65.48 | N |
| ATOM | 33 | CA | TYR | A | 19 | 57.389 | 49.043 | 97.057 | 1.00 | 65.58 | C |
| ATOM | 34 | CB | TYR | A | 19 | 58.325 | 48.355 | 96.092 | 1.00 | 67.12 | C |
| ATOM | 35 | CG | TYR | A | 19 | 59.747 | 48.256 | 96.555 | 1.00 | 69.35 | C |
| ATOM | 36 | CD1 | TYR | A | 19 | 60.404 | 49.357 | 97.110 | 1.00 | 69.97 | C |
| ATOM | 37 | CE1 | TYR | A | 19 | 61.772 | 49.324 | 97.346 | 1.00 | 71.15 | C |
| ATOM | 38 | CD2 | TYR | A | 19 | 60.495 | 47.114 | 96.275 | 1.00 | 70.44 | C |
| ATOM | 39 | CE2 | TYR | A | 19 | 61.856 | 47.071 | 96.510 | 1.00 | 71.26 | C |
| ATOM | 40 | CZ | TYR | A | 19 | 62.490 | 48.176 | 97.032 | 1.00 | 71.50 | C |
| ATOM | 41 | OH | TYR | A | 19 | 63.859 | 48.133 | 97.159 | 1.00 | 72.36 | O |
| ATOM | 42 | C | TYR | A | 19 | 56.023 | 49.150 | 96.399 | 1.00 | 64.97 | C |
| ATOM | 43 | O | TYR | A | 19 | 55.569 | 50.231 | 96.026 | 1.00 | 64.86 | O |
| ATOM | 44 | N | ILE | A | 20 | 55.383 | 48.000 | 96.244 | 1.00 | 64.36 | N |
| ATOM | 45 | CA | ILE | A | 20 | 54.069 | 47.932 | 95.638 | 1.00 | 64.10 | C |
| ATOM | 46 | CB | ILE | A | 20 | 53.521 | 46.500 | 95.694 | 1.00 | 62.75 | C |
| ATOM | 47 | CG2 | ILE | A | 20 | 52.119 | 46.459 | 95.136 | 1.00 | 61.94 | C |
| ATOM | 48 | CG1 | ILE | A | 20 | 54.442 | 45.567 | 94.899 | 1.00 | 62.06 | C |
| ATOM | 49 | CD1 | ILE | A | 20 | 54.147 | 44.095 | 95.093 | 1.00 | 59.61 | C |
| ATOM | 50 | C | ILE | A | 20 | 53.118 | 48.862 | 96.368 | 1.00 | 64.66 | C |
| ATOM | 51 | O | ILE | A | 20 | 52.395 | 49.621 | 95.741 | 1.00 | 65.70 | O |
| ATOM | 52 | N | GLU | A | 21 | 53.136 | 48.806 | 97.694 | 1.00 | 65.14 | N |
| ATOM | 53 | CA | GLU | A | 21 | 52.275 | 49.641 | 98.524 | 1.00 | 65.65 | C |
| ATOM | 54 | CB | GLU | A | 21 | 52.511 | 49.307 | 100.000 | 1.00 | 67.52 | C |
| ATOM | 55 | CG | GLU | A | 21 | 51.775 | 50.210 | 100.981 | 1.00 | 69.57 | C |
| ATOM | 56 | CD | GLU | A | 21 | 50.288 | 50.272 | 100.696 | 1.00 | 71.31 | C |
| ATOM | 57 | OE1 | GLU | A | 21 | 49.643 | 49.201 | 100.640 | 1.00 | 71.13 | O |
| ATOM | 58 | OE2 | GLU | A | 21 | 49.769 | 51.396 | 100.528 | 1.00 | 72.55 | O |
| ATOM | 59 | C | GLU | A | 21 | 52.523 | 51.128 | 98.295 | 1.00 | 65.18 | C |
| ATOM | 60 | O | GLU | A | 21 | 51.615 | 51.950 | 98.414 | 1.00 | 65.35 | O |
| ATOM | 61 | N | HIS | A | 22 | 53.764 | 51.461 | 97.966 | 1.00 | 64.15 | N |
| ATOM | 62 | CA | HIS | A | 22 | 54.164 | 52.838 | 97.743 | 1.00 | 63.48 | C |
| ATOM | 63 | CB | HIS | A | 22 | 55.680 | 52.956 | 97.853 | 1.00 | 62.91 | C |
| ATOM | 64 | CG | HIS | A | 22 | 56.194 | 54.331 | 97.573 | 1.00 | 62.69 | C |
| ATOM | 65 | CD2 | HIS | A | 22 | 57.030 | 54.794 | 96.615 | 1.00 | 62.65 | C |
| ATOM | 66 | ND1 | HIS | A | 22 | 55.828 | 55.427 | 98.326 | 1.00 | 62.37 | N |
| ATOM | 67 | CE1 | HIS | A | 22 | 56.417 | 56.505 | 97.842 | 1.00 | 62.72 | C |
| ATOM | 68 | NE2 | HIS | A | 22 | 57.152 | 56.148 | 96.803 | 1.00 | 62.91 | N |
| ATOM | 69 | C | HIS | A | 22 | 53.717 | 53.398 | 96.405 | 1.00 | 63.30 | C |
| ATOM | 70 | O | HIS | A | 22 | 52.930 | 54.338 | 96.354 | 1.00 | 62.89 | O |
| ATOM | 71 | N | PHE | A | 23 | 54.242 | 52.832 | 95.323 | 1.00 | 63.71 | N |
| ATOM | 72 | CA | PHE | A | 23 | 53.891 | 53.292 | 93.987 | 1.00 | 63.91 | C |
| ATOM | 73 | CB | PHE | A | 23 | 54.783 | 52.617 | 92.947 | 1.00 | 63.16 | C |
| ATOM | 74 | CG | PHE | A | 23 | 56.236 | 52.949 | 93.101 | 1.00 | 63.15 | C |
| ATOM | 75 | CD1 | PHE | A | 23 | 57.090 | 52.103 | 93.809 | 1.00 | 63.26 | C |
| ATOM | 76 | CD2 | PHE | A | 23 | 56.749 | 54.130 | 92.570 | 1.00 | 62.79 | C |
| ATOM | 77 | CE1 | PHE | A | 23 | 58.440 | 52.430 | 93.987 | 1.00 | 62.91 | C |
| ATOM | 78 | CE2 | PHE | A | 23 | 58.093 | 54.468 | 92.742 | 1.00 | 62.82 | C |
| ATOM | 79 | CZ | PHE | A | 23 | 58.940 | 53.614 | 93.453 | 1.00 | 62.52 | C |
| ATOM | 80 | C | PHE | A | 23 | 52.416 | 53.039 | 93.683 | 1.00 | 64.20 | C |
| ATOM | 81 | O | PHE | A | 23 | 51.855 | 53.629 | 92.765 | 1.00 | 63.83 | O |
| ATOM | 82 | N | SER | A | 24 | 51.794 | 52.171 | 94.475 | 1.00 | 65.24 | N |
| ATOM | 83 | CA | SER | A | 24 | 50.383 | 51.840 | 94.316 | 1.00 | 65.77 | C |
| ATOM | 84 | CB | SER | A | 24 | 50.035 | 50.602 | 95.145 | 1.00 | 66.11 | C |
| ATOM | 85 | OG | SER | A | 24 | 48.636 | 50.436 | 95.269 | 1.00 | 67.63 | O |
| ATOM | 86 | C | SER | A | 24 | 49.528 | 53.004 | 94.782 | 1.00 | 65.99 | C |
| ATOM | 87 | O | SER | A | 24 | 48.305 | 52.983 | 94.658 | 1.00 | 65.59 | O |
| ATOM | 88 | N | LYS | A | 25 | 50.179 | 54.017 | 95.335 | 1.00 | 66.47 | N |
| ATOM | 89 | CA | LYS | A | 25 | 49.463 | 55.184 | 95.830 | 1.00 | 67.44 | C |
| ATOM | 90 | CB | LYS | A | 25 | 50.232 | 55.810 | 96.998 | 1.00 | 68.99 | C |
| ATOM | 91 | CG | LYS | A | 25 | 50.634 | 54.806 | 98.080 | 1.00 | 71.13 | C |
| ATOM | 92 | CD | LYS | A | 25 | 49.954 | 55.116 | 99.409 | 1.00 | 73.16 | C |
| ATOM | 93 | CE | LYS | A | 25 | 49.167 | 53.918 | 99.936 | 1.00 | 73.82 | C |
| ATOM | 94 | NZ | LYS | A | 25 | 48.520 | 53.134 | 98.834 | 1.00 | 75.51 | N |
| ATOM | 95 | C | LYS | A | 25 | 49.310 | 56.176 | 94.685 | 1.00 | 66.70 | C |
| ATOM | 96 | O | LYS | A | 25 | 48.327 | 56.908 | 94.611 | 1.00 | 66.94 | O |
| ATOM | 97 | N | PHE | A | 26 | 50.290 | 56.186 | 93.788 | 1.00 | 65.63 | N |
| ATOM | 98 | CA | PHE | A | 26 | 50.253 | 57.071 | 92.634 | 1.00 | 64.25 | C |
| ATOM | 99 | CB | PHE | A | 26 | 51.645 | 57.201 | 92.013 | 1.00 | 65.96 | C |
| ATOM | 100 | CG | PHE | A | 26 | 52.676 | 57.785 | 92.938 | 1.00 | 69.07 | C |
| ATOM | 101 | CD1 | PHE | A | 26 | 53.129 | 57.070 | 94.049 | 1.00 | 69.43 | C |
| ATOM | 102 | CD2 | PHE | A | 26 | 53.201 | 59.058 | 92.700 | 1.00 | 69.67 | C |
| ATOM | 103 | CE1 | PHE | A | 26 | 54.084 | 57.614 | 94.908 | 1.00 | 70.24 | C |
| ATOM | 104 | CE2 | PHE | A | 26 | 54.158 | 59.611 | 93.555 | 1.00 | 70.17 | C |
| ATOM | 105 | CZ | PHE | A | 26 | 54.600 | 58.888 | 94.660 | 1.00 | 70.20 | C |
| ATOM | 106 | C | PHE | A | 26 | 49.298 | 56.500 | 91.584 | 1.00 | 62.51 | C |
| ATOM | 107 | O | PHE | A | 26 | 48.956 | 55.317 | 91.617 | 1.00 | 61.70 | O |
| ATOM | 108 | N | SER | A | 27 | 48.855 | 57.353 | 90.666 | 1.00 | 60.28 | N |
| ATOM | 109 | CA | SER | A | 27 | 47.984 | 56.925 | 89.581 | 1.00 | 57.03 | C |
| ATOM | 110 | CB | SER | A | 27 | 46.766 | 57.838 | 89.457 | 1.00 | 57.46 | C |
| ATOM | 111 | OG | SER | A | 27 | 45.774 | 57.511 | 90.412 | 1.00 | 58.60 | O |
| ATOM | 112 | C | SER | A | 27 | 48.825 | 57.019 | 88.319 | 1.00 | 54.97 | C |
| ATOM | 113 | O | SER | A | 27 | 49.658 | 57.909 | 88.193 | 1.00 | 54.19 | O |
| ATOM | 114 | N | PRO | A | 28 | 48.651 | 56.078 | 87.382 | 1.00 | 53.39 | N |
| ATOM | 115 | CD | PRO | A | 28 | 47.870 | 54.828 | 87.434 | 1.00 | 52.63 | C |
| ATOM | 116 | CA | PRO | A | 28 | 49.454 | 56.158 | 86.158 | 1.00 | 51.94 | C |
| ATOM | 117 | CB | PRO | A | 28 | 49.125 | 54.846 | 85.440 | 1.00 | 52.88 | C |
| ATOM | 118 | CG | PRO | A | 28 | 47.764 | 54.471 | 85.988 | 1.00 | 53.00 | C |
| ATOM | 119 | C | PRO | A | 28 | 49.085 | 57.404 | 85.356 | 1.00 | 51.25 | C |
| ATOM | 120 | O | PRO | A | 28 | 47.945 | 57.861 | 85.413 | 1.00 | 50.50 | O |
| ATOM | 121 | N | SER | A | 29 | 50.051 | 57.952 | 84.621 | 1.00 | 50.11 | N |
| ATOM | 122 | CA | SER | A | 29 | 49.833 | 59.162 | 83.826 | 1.00 | 48.66 | C |
| ATOM | 123 | CB | SER | A | 29 | 51.047 | 60.083 | 83.936 | 1.00 | 49.04 | C |
| ATOM | 124 | OG | SER | A | 29 | 51.165 | 60.594 | 85.247 | 1.00 | 52.11 | O |
| ATOM | 125 | C | SER | A | 29 | 49.540 | 58.907 | 82.356 | 1.00 | 47.26 | C |
| ATOM | 126 | O | SER | A | 29 | 50.437 | 58.627 | 81.570 | 1.00 | 45.95 | O |
| ATOM | 127 | N | PRO | A | 30 | 48.274 | 59.028 | 81.960 | 1.00 | 46.37 | N |
| ATOM | 128 | CD | PRO | A | 30 | 47.057 | 59.272 | 82.753 | 1.00 | 46.65 | C |
| ATOM | 129 | CA | PRO | A | 30 | 47.961 | 58.788 | 80.550 | 1.00 | 47.05 | C |
| ATOM | 130 | CB | PRO | A | 30 | 46.429 | 58.870 | 80.508 | 1.00 | 46.28 | C |
| ATOM | 131 | CG | PRO | A | 30 | 46.080 | 59.713 | 81.702 | 1.00 | 46.98 | C |
| ATOM | 132 | C | PRO | A | 30 | 48.649 | 59.799 | 79.641 | 1.00 | 47.44 | C |
| ATOM | 133 | O | PRO | A | 30 | 48.597 | 60.999 | 79.891 | 1.00 | 48.20 | O |
| ATOM | 134 | N | LEU | A | 31 | 49.314 | 59.298 | 78.604 | 1.00 | 47.08 | N |
| ATOM | 135 | CA | LEU | A | 31 | 50.025 | 60.126 | 77.640 | 1.00 | 47.11 | C |
| ATOM | 136 | CB | LEU | A | 31 | 51.411 | 59.542 | 77.359 | 1.00 | 46.19 | C |
| ATOM | 137 | CG | LEU | A | 31 | 52.299 | 59.182 | 78.555 | 1.00 | 47.20 | C |
| ATOM | 138 | CD1 | LEU | A | 31 | 53.520 | 58.400 | 78.087 | 1.00 | 46.84 | C |
| ATOM | 139 | CD2 | LEU | A | 31 | 52.721 | 60.454 | 79.266 | 1.00 | 47.92 | C |
| ATOM | 140 | C | LEU | A | 31 | 49.236 | 60.183 | 76.331 | 1.00 | 48.65 | C |
| ATOM | 141 | O | LEU | A | 31 | 48.551 | 59.226 | 75.964 | 1.00 | 48.47 | O |
| ATOM | 142 | N | SER | A | 32 | 49.339 | 61.303 | 75.621 | 1.00 | 49.53 | N |
| ATOM | 143 | CA | SER | A | 32 | 48.629 | 61.457 | 74.358 | 1.00 | 51.10 | C |
| ATOM | 144 | CB | SER | A | 32 | 48.103 | 62.876 | 74.211 | 1.00 | 50.68 | C |
| ATOM | 145 | OG | SER | A | 32 | 49.169 | 63.751 | 73.895 | 1.00 | 50.86 | O |
| ATOM | 146 | C | SER | A | 32 | 49.606 | 61.179 | 73.235 | 1.00 | 52.53 | C |
| ATOM | 147 | O | SER | A | 32 | 50.817 | 61.339 | 73.400 | 1.00 | 52.71 | O |
| ATOM | 148 | N | MET | A | 33 | 49.087 | 60.771 | 72.088 | 1.00 | 54.66 | N |
| ATOM | 149 | CA | MET | A | 33 | 49.958 | 60.473 | 70.972 | 1.00 | 57.48 | C |
| ATOM | 150 | CB | MET | A | 33 | 49.168 | 59.878 | 69.816 | 1.00 | 58.52 | C |
| ATOM | 151 | CG | MET | A | 33 | 50.045 | 59.241 | 68.770 | 1.00 | 60.00 | C |
| ATOM | 152 | SD | MET | A | 33 | 49.649 | 57.524 | 68.604 | 1.00 | 63.43 | S |
| ATOM | 153 | CE | MET | A | 33 | 47.900 | 57.598 | 68.396 | 1.00 | 62.79 | C |
| ATOM | 154 | C | MET | A | 33 | 50.730 | 61.693 | 70.492 | 1.00 | 58.79 | C |
| ATOM | 155 | O | MET | A | 33 | 51.767 | 61.550 | 69.852 | 1.00 | 59.14 | O |
| ATOM | 156 | N | LYS | A | 34 | 50.242 | 62.896 | 70.778 | 1.00 | 60.41 | N |
| ATOM | 157 | CA | LYS | A | 34 | 51.006 | 64.056 | 70.348 | 1.00 | 61.71 | C |
| ATOM | 158 | CB | LYS | A | 34 | 50.166 | 65.343 | 70.328 | 1.00 | 63.58 | C |
| ATOM | 159 | CG | LYS | A | 34 | 50.529 | 66.222 | 69.117 | 1.00 | 65.13 | C |
| ATOM | 160 | CD | LYS | A | 34 | 49.635 | 67.442 | 68.928 | 1.00 | 67.35 | C |
| ATOM | 161 | CE | LYS | A | 34 | 50.051 | 68.594 | 69.833 | 1.00 | 68.51 | C |
| ATOM | 162 | NZ | LYS | A | 34 | 48.993 | 68.941 | 70.823 | 1.00 | 70.29 | N |
| ATOM | 163 | C | LYS | A | 34 | 52.202 | 64.203 | 71.280 | 1.00 | 61.51 | C |
| ATOM | 164 | O | LYS | A | 34 | 53.283 | 64.577 | 70.843 | 1.00 | 62.24 | O |
| ATOM | 165 | N | GLN | A | 35 | 52.014 | 63.890 | 72.560 | 1.00 | 61.56 | N |
| ATOM | 166 | CA | GLN | A | 35 | 53.114 | 63.958 | 73.521 | 1.00 | 61.69 | C |
| ATOM | 167 | CB | GLN | A | 35 | 52.626 | 63.609 | 74.921 | 1.00 | 61.57 | C |
| ATOM | 168 | CG | GLN | A | 35 | 51.511 | 64.486 | 75.420 | 1.00 | 62.12 | C |
| ATOM | 169 | CD | GLN | A | 35 | 51.136 | 64.166 | 76.851 | 1.00 | 62.64 | C |
| ATOM | 170 | OE1 | GLN | A | 35 | 50.179 | 63.431 | 77.111 | 1.00 | 63.28 | O |
| ATOM | 171 | NE2 | GLN | A | 35 | 51.902 | 64.706 | 77.793 | 1.00 | 61.52 | N |
| ATOM | 172 | C | GLN | A | 35 | 54.206 | 62.961 | 73.113 | 1.00 | 61.99 | C |
| ATOM | 173 | O | GLN | A | 35 | 55.391 | 63.168 | 73.394 | 1.00 | 62.09 | O |
| ATOM | 174 | N | PHE | A | 36 | 53.792 | 61.875 | 72.463 | 1.00 | 62.22 | N |
| ATOM | 175 | CA | PHE | A | 36 | 54.715 | 60.851 | 71.996 | 1.00 | 62.77 | C |
| ATOM | 176 | CB | PHE | A | 36 | 53.960 | 59.568 | 71.614 | 1.00 | 60.68 | C |
| ATOM | 177 | CG | PHE | A | 36 | 53.594 | 58.679 | 72.780 | 1.00 | 58.71 | C |
| ATOM | 178 | CD1 | PHE | A | 36 | 52.257 | 58.458 | 73.115 | 1.00 | 57.72 | C |
| ATOM | 179 | CD2 | PHE | A | 36 | 54.580 | 58.018 | 73.506 | 1.00 | 57.37 | C |
| ATOM | 180 | CE1 | PHE | A | 36 | 51.909 | 57.591 | 74.151 | 1.00 | 56.50 | C |
| ATOM | 181 | CE2 | PHE | A | 36 | 54.238 | 57.148 | 74.543 | 1.00 | 57.11 | C |
| ATOM | 182 | CZ | PHE | A | 36 | 52.904 | 56.935 | 74.865 | 1.00 | 56.70 | C |
| ATOM | 183 | C | PHE | A | 36 | 55.450 | 61.372 | 70.768 | 1.00 | 64.55 | C |
| ATOM | 184 | O | PHE | A | 36 | 56.647 | 61.152 | 70.615 | 1.00 | 64.51 | O |
| ATOM | 185 | N | LEU | A | 37 | 54.712 | 62.057 | 69.897 | 1.00 | 67.44 | N |
| ATOM | 186 | CA | LEU | A | 37 | 55.249 | 62.624 | 68.659 | 1.00 | 70.20 | C |
| ATOM | 187 | CB | LEU | A | 37 | 54.108 | 63.017 | 67.709 | 1.00 | 70.08 | C |
| ATOM | 188 | CG | LEU | A | 37 | 53.865 | 62.203 | 66.435 | 1.00 | 70.45 | C |
| ATOM | 189 | CD1 | LEU | A | 37 | 52.829 | 62.922 | 65.578 | 1.00 | 70.89 | C |
| ATOM | 190 | CD2 | LEU | A | 37 | 55.149 | 62.049 | 65.651 | 1.00 | 70.62 | C |
| ATOM | 191 | C | LEU | A | 37 | 56.124 | 63.852 | 68.896 | 1.00 | 72.01 | C |
| ATOM | 192 | O | LEU | A | 37 | 57.218 | 63.958 | 68.335 | 1.00 | 72.61 | O |
| ATOM | 193 | N | ASP | A | 38 | 55.629 | 64.785 | 69.709 | 1.00 | 73.97 | N |
| ATOM | 194 | CA | ASP | A | 38 | 56.361 | 66.015 | 70.024 | 1.00 | 75.77 | C |
| ATOM | 195 | CB | ASP | A | 38 | 55.464 | 67.016 | 70.766 | 1.00 | 76.31 | C |
| ATOM | 196 | CG | ASP | A | 38 | 54.237 | 67.413 | 69.966 | 1.00 | 77.29 | C |
| ATOM | 197 | OD1 | ASP | A | 38 | 54.188 | 67.121 | 68.750 | 1.00 | 78.24 | O |
| ATOM | 198 | OD2 | ASP | A | 38 | 53.321 | 68.027 | 70.554 | 1.00 | 77.97 | O |
| ATOM | 199 | C | ASP | A | 38 | 57.543 | 65.664 | 70.913 | 1.00 | 76.42 | C |
| ATOM | 200 | O | ASP | A | 38 | 57.849 | 66.378 | 71.874 | 1.00 | 76.84 | O |
| ATOM | 201 | N | PHE | A | 39 | 58.210 | 64.565 | 70.579 | 1.00 | 76.70 | N |
| ATOM | 202 | CA | PHE | A | 39 | 59.339 | 64.097 | 71.361 | 1.00 | 76.70 | C |
| ATOM | 203 | CB | PHE | A | 39 | 58.871 | 62.954 | 72.254 | 1.00 | 76.32 | C |
| ATOM | 204 | CG | PHE | A | 39 | 59.851 | 62.575 | 73.304 | 1.00 | 75.36 | C |
| ATOM | 205 | CD1 | PHE | A | 39 | 59.665 | 62.975 | 74.616 | 1.00 | 75.24 | C |
| ATOM | 206 | CD2 | PHE | A | 39 | 60.978 | 61.839 | 72.980 | 1.00 | 75.05 | C |
| ATOM | 207 | CE1 | PHE | A | 39 | 60.591 | 62.650 | 75.594 | 1.00 | 75.12 | C |
| ATOM | 208 | CE2 | PHE | A | 39 | 61.910 | 61.509 | 73.950 | 1.00 | 74.94 | C |
| ATOM | 209 | CZ | PHE | A | 39 | 61.717 | 61.915 | 75.260 | 1.00 | 74.47 | C |
| ATOM | 210 | C | PHE | A | 39 | 60.508 | 63.635 | 70.495 | 1.00 | 77.15 | C |
| ATOM | 211 | O | PHE | A | 39 | 60.472 | 63.720 | 69.269 | 1.00 | 77.69 | O |
| ATOM | 212 | N | SER | A | 42 | 66.323 | 61.416 | 67.582 | 1.00 | 99.35 | N |
| ATOM | 213 | CA | SER | A | 42 | 67.146 | 60.697 | 68.547 | 1.00 | 99.43 | C |
| ATOM | 214 | CB | SER | A | 42 | 68.173 | 59.818 | 67.824 | 1.00 | 99.22 | C |
| ATOM | 215 | OG | SER | A | 42 | 69.066 | 59.220 | 68.748 | 1.00 | 98.72 | O |
| ATOM | 216 | C | SER | A | 42 | 67.874 | 61.641 | 69.496 | 1.00 | 99.51 | C |
| ATOM | 217 | O | SER | A | 42 | 68.816 | 62.331 | 69.097 | 1.00 | 99.93 | O |
| ATOM | 218 | N | ASN | A | 43 | 67.417 | 61.660 | 70.746 | 1.00 | 98.96 | N |
| ATOM | 219 | CA | ASN | A | 43 | 67.980 | 62.474 | 71.823 | 1.00 | 98.40 | C |
| ATOM | 220 | CB | ASN | A | 43 | 69.451 | 62.840 | 71.550 | 1.00 | 98.91 | C |
| ATOM | 221 | CG | ASN | A | 43 | 70.167 | 63.374 | 72.792 | 1.00 | 99.29 | C |
| ATOM | 222 | OD1 | ASN | A | 43 | 69.603 | 63.402 | 73.890 | 1.00 | 98.95 | O |
| ATOM | 223 | ND2 | ASN | A | 43 | 71.418 | 63.793 | 72.618 | 1.00 | 99.52 | N |
| ATOM | 224 | C | ASN | A | 43 | 67.202 | 63.738 | 72.181 | 1.00 | 97.80 | C |
| ATOM | 225 | O | ASN | A | 43 | 67.773 | 64.829 | 72.281 | 1.00 | 97.76 | O |
| ATOM | 226 | N | ALA | A | 44 | 65.890 | 63.589 | 72.366 | 1.00 | 96.48 | N |
| ATOM | 227 | CA | ALA | A | 44 | 65.058 | 64.702 | 72.817 | 1.00 | 94.90 | C |
| ATOM | 228 | CB | ALA | A | 44 | 63.603 | 64.466 | 72.468 | 1.00 | 95.10 | C |
| ATOM | 229 | C | ALA | A | 44 | 65.293 | 64.472 | 74.301 | 1.00 | 93.86 | C |
| ATOM | 230 | O | ALA | A | 44 | 64.439 | 64.727 | 75.153 | 1.00 | 93.73 | O |
| ATOM | 231 | N | CYS | A | 45 | 66.496 | 63.957 | 74.554 | 1.00 | 92.36 | N |
| ATOM | 232 | CA | CYS | A | 45 | 67.000 | 63.582 | 75.858 | 1.00 | 90.49 | C |
| ATOM | 233 | CB | CYS | A | 45 | 66.813 | 64.703 | 76.882 | 1.00 | 90.84 | C |
| ATOM | 234 | SG | CYS | A | 45 | 68.145 | 65.917 | 76.853 | 1.00 | 92.15 | S |
| ATOM | 235 | C | CYS | A | 45 | 66.282 | 62.335 | 76.313 | 1.00 | 88.65 | C |
| ATOM | 236 | O | CYS | A | 45 | 65.054 | 62.281 | 76.327 | 1.00 | 88.36 | O |
| ATOM | 237 | N | GLU | A | 46 | 67.056 | 61.309 | 76.629 | 1.00 | 86.27 | N |
| ATOM | 238 | CA | GLU | A | 46 | 66.472 | 60.089 | 77.126 | 1.00 | 84.14 | C |
| ATOM | 239 | CB | GLU | A | 46 | 67.176 | 58.860 | 76.564 | 1.00 | 84.32 | C |
| ATOM | 240 | CG | GLU | A | 46 | 68.667 | 58.947 | 76.456 | 1.00 | 83.92 | C |
| ATOM | 241 | CD | GLU | A | 46 | 69.270 | 57.580 | 76.219 | 1.00 | 84.15 | C |
| ATOM | 242 | OE1 | GLU | A | 46 | 68.935 | 56.660 | 76.996 | 1.00 | 84.17 | O |
| ATOM | 243 | OE2 | GLU | A | 46 | 70.066 | 57.418 | 75.269 | 1.00 | 84.11 | O |
| ATOM | 244 | C | GLU | A | 46 | 66.653 | 60.194 | 78.618 | 1.00 | 82.57 | C |
| ATOM | 245 | O | GLU | A | 46 | 66.161 | 59.374 | 79.385 | 1.00 | 82.00 | O |
| ATOM | 246 | N | LYS | A | 47 | 67.381 | 61.230 | 79.017 | 1.00 | 80.90 | N |
| ATOM | 247 | CA | LYS | A | 47 | 67.593 | 61.499 | 80.424 | 1.00 | 79.18 | C |
| ATOM | 248 | CB | LYS | A | 47 | 68.539 | 62.694 | 80.608 | 1.00 | 80.36 | C |
| ATOM | 249 | CG | LYS | A | 47 | 69.884 | 62.569 | 79.887 | 1.00 | 81.19 | C |
| ATOM | 250 | CD | LYS | A | 47 | 70.813 | 63.750 | 80.201 | 1.00 | 82.42 | C |
| ATOM | 251 | CE | LYS | A | 47 | 70.213 | 65.088 | 79.764 | 1.00 | 82.76 | C |
| ATOM | 252 | NZ | LYS | A | 47 | 71.107 | 66.240 | 80.087 | 1.00 | 82.77 | N |
| ATOM | 253 | C | LYS | A | 47 | 66.182 | 61.871 | 80.867 | 1.00 | 77.21 | C |
| ATOM | 254 | O | LYS | A | 47 | 65.695 | 61.421 | 81.905 | 1.00 | 76.52 | O |
| ATOM | 255 | N | THR | A | 48 | 65.527 | 62.682 | 80.038 | 1.00 | 75.12 | N |
| ATOM | 256 | CA | THR | A | 48 | 64.164 | 63.137 | 80.290 | 1.00 | 73.39 | C |
| ATOM | 257 | CB | THR | A | 48 | 63.685 | 64.108 | 79.196 | 1.00 | 73.80 | C |
| ATOM | 258 | OG1 | THR | A | 48 | 64.562 | 65.239 | 79.135 | 1.00 | 74.10 | O |
| ATOM | 259 | CG2 | THR | A | 48 | 62.274 | 64.583 | 79.492 | 1.00 | 73.39 | C |
| ATOM | 260 | C | THR | A | 48 | 63.212 | 61.949 | 80.322 | 1.00 | 71.83 | C |
| ATOM | 261 | O | THR | A | 48 | 62.256 | 61.929 | 81.091 | 1.00 | 71.67 | O |
| ATOM | 262 | N | SER | A | 49 | 63.478 | 60.963 | 79.476 | 1.00 | 70.01 | N |
| ATOM | 263 | CA | SER | A | 49 | 62.655 | 59.760 | 79.423 | 1.00 | 68.53 | C |
| ATOM | 264 | CB | SER | A | 49 | 62.884 | 59.021 | 78.102 | 1.00 | 68.59 | C |
| ATOM | 265 | OG | SER | A | 49 | 62.203 | 57.781 | 78.089 | 1.00 | 67.16 | O |
| ATOM | 266 | C | SER | A | 49 | 63.019 | 58.850 | 80.589 | 1.00 | 67.55 | C |
| ATOM | 267 | O | SER | A | 49 | 62.145 | 58.285 | 81.248 | 1.00 | 67.87 | O |
| ATOM | 268 | N | PHE | A | 50 | 64.320 | 58.717 | 80.833 | 1.00 | 66.00 | N |
| ATOM | 269 | CA | PHE | A | 50 | 64.829 | 57.891 | 81.920 | 1.00 | 63.98 | C |
| ATOM | 270 | CB | PHE | A | 50 | 66.352 | 58.017 | 82.013 | 1.00 | 64.09 | C |
| ATOM | 271 | CG | PHE | A | 50 | 66.927 | 57.446 | 83.276 | 1.00 | 64.15 | C |
| ATOM | 272 | CD1 | PHE | A | 50 | 67.006 | 56.070 | 83.460 | 1.00 | 63.61 | C |
| ATOM | 273 | CD2 | PHE | A | 50 | 67.357 | 58.288 | 84.300 | 1.00 | 63.72 | C |
| ATOM | 274 | CE1 | PHE | A | 50 | 67.502 | 55.534 | 84.647 | 1.00 | 63.66 | C |
| ATOM | 275 | CE2 | PHE | A | 50 | 67.854 | 57.764 | 85.487 | 1.00 | 64.56 | C |
| ATOM | 276 | CZ | PHE | A | 50 | 67.927 | 56.382 | 85.663 | 1.00 | 64.23 | C |
| ATOM | 277 | C | PHE | A | 50 | 64.210 | 58.299 | 83.250 | 1.00 | 62.66 | C |
| ATOM | 278 | O | PHE | A | 50 | 63.697 | 57.458 | 83.982 | 1.00 | 62.53 | O |
| ATOM | 279 | N | THR | A | 51 | 64.266 | 59.591 | 83.558 | 1.00 | 61.36 | N |
| ATOM | 280 | CA | THR | A | 51 | 63.719 | 60.100 | 84.810 | 1.00 | 60.70 | C |
| ATOM | 281 | CB | THR | A | 51 | 64.091 | 61.584 | 85.033 | 1.00 | 61.10 | C |
| ATOM | 282 | OG1 | THR | A | 51 | 63.906 | 62.314 | 83.816 | 1.00 | 62.60 | O |
| ATOM | 283 | CG2 | THR | A | 51 | 65.542 | 61.710 | 85.463 | 1.00 | 61.75 | C |
| ATOM | 284 | C | THR | A | 51 | 62.208 | 59.951 | 84.863 | 1.00 | 59.36 | C |
| ATOM | 285 | O | THR | A | 51 | 61.631 | 59.812 | 85.941 | 1.00 | 58.79 | O |
| ATOM | 286 | N | PHE | A | 52 | 61.563 | 59.986 | 83.701 | 1.00 | 58.39 | N |
| ATOM | 287 | CA | PHE | A | 52 | 60.120 | 59.825 | 83.659 | 1.00 | 57.35 | C |
| ATOM | 288 | CB | PHE | A | 52 | 59.539 | 60.322 | 82.339 | 1.00 | 57.63 | C |
| ATOM | 289 | CG | PHE | A | 52 | 58.086 | 59.950 | 82.152 | 1.00 | 57.77 | C |
| ATOM | 290 | CD1 | PHE | A | 52 | 57.079 | 60.632 | 82.833 | 1.00 | 57.69 | C |
| ATOM | 291 | CD2 | PHE | A | 52 | 57.731 | 58.879 | 81.337 | 1.00 | 57.52 | C |
| ATOM | 292 | CE1 | PHE | A | 52 | 55.739 | 60.254 | 82.705 | 1.00 | 58.51 | C |
| ATOM | 293 | CE2 | PHE | A | 52 | 56.393 | 58.491 | 81.201 | 1.00 | 58.57 | C |
| ATOM | 294 | CZ | PHE | A | 52 | 55.395 | 59.179 | 81.885 | 1.00 | 58.47 | C |
| ATOM | 295 | C | PHE | A | 52 | 59.728 | 58.363 | 83.831 | 1.00 | 56.84 | C |
| ATOM | 296 | O | PHE | A | 52 | 58.812 | 58.046 | 84.588 | 1.00 | 56.23 | O |
| ATOM | 297 | N | LEU | A | 53 | 60.413 | 57.472 | 83.121 | 1.00 | 57.18 | N |
| ATOM | 298 | CA | LEU | A | 53 | 60.099 | 56.046 | 83.195 | 1.00 | 58.75 | C |
| ATOM | 299 | CB | LEU | A | 53 | 60.778 | 55.291 | 82.041 | 1.00 | 57.67 | C |
| ATOM | 300 | CG | LEU | A | 53 | 60.238 | 55.641 | 80.650 | 1.00 | 57.83 | C |
| ATOM | 301 | CD1 | LEU | A | 53 | 60.976 | 54.850 | 79.578 | 1.00 | 57.63 | C |
| ATOM | 302 | CD2 | LEU | A | 53 | 58.740 | 55.351 | 80.599 | 1.00 | 56.72 | C |
| ATOM | 303 | C | LEU | A | 53 | 60.447 | 55.390 | 84.526 | 1.00 | 59.85 | C |
| ATOM | 304 | O | LEU | A | 53 | 59.836 | 54.383 | 84.896 | 1.00 | 60.63 | O |
| ATOM | 305 | N | ARG | A | 54 | 61.417 | 55.961 | 85.243 | 1.00 | 61.34 | N |
| ATOM | 306 | CA | ARG | A | 54 | 61.842 | 55.446 | 86.553 | 1.00 | 61.84 | C |
| ATOM | 307 | CB | ARG | A | 54 | 62.963 | 56.295 | 87.130 | 1.00 | 64.80 | C |
| ATOM | 308 | CG | ARG | A | 54 | 64.297 | 56.160 | 86.472 | 1.00 | 68.88 | C |
| ATOM | 309 | CD | ARG | A | 54 | 65.156 | 57.349 | 86.870 | 1.00 | 72.21 | C |
| ATOM | 310 | NE | ARG | A | 54 | 65.017 | 57.704 | 88.280 | 1.00 | 74.67 | N |
| ATOM | 311 | CZ | ARG | A | 54 | 65.822 | 58.551 | 88.917 | 1.00 | 76.48 | C |
| ATOM | 312 | NH1 | ARG | A | 54 | 66.830 | 59.130 | 88.271 | 1.00 | 76.94 | N |
| ATOM | 313 | NH2 | ARG | A | 54 | 65.618 | 58.820 | 90.203 | 1.00 | 77.01 | N |
| ATOM | 314 | C | ARG | A | 54 | 60.699 | 55.510 | 87.548 | 1.00 | 61.04 | C |
| ATOM | 315 | O | ARG | A | 54 | 60.618 | 54.697 | 88.467 | 1.00 | 60.48 | O |
| ATOM | 316 | N | GLN | A | 55 | 59.839 | 56.510 | 87.375 | 1.00 | 59.12 | N |
| ATOM | 317 | CA | GLN | A | 55 | 58.699 | 56.709 | 88.257 | 1.00 | 58.51 | C |
| ATOM | 318 | CB | GLN | A | 55 | 58.464 | 58.208 | 88.465 | 1.00 | 60.57 | C |
| ATOM | 319 | CG | GLN | A | 55 | 59.667 | 58.946 | 89.003 | 1.00 | 64.55 | C |
| ATOM | 320 | CD | GLN | A | 55 | 59.828 | 58.752 | 90.493 | 1.00 | 66.62 | C |
| ATOM | 321 | OE1 | GLN | A | 55 | 60.901 | 59.001 | 91.051 | 1.00 | 67.90 | O |
| ATOM | 322 | NE2 | GLN | A | 55 | 58.751 | 58.314 | 91.154 | 1.00 | 66.75 | N |
| ATOM | 323 | C | GLN | A | 55 | 57.425 | 56.088 | 87.689 | 1.00 | 56.35 | C |
| ATOM | 324 | O | GLN | A | 55 | 56.667 | 55.433 | 88.405 | 1.00 | 55.70 | O |
| ATOM | 325 | N | GLU | A | 56 | 57.200 | 56.300 | 86.395 | 1.00 | 54.05 | N |
| ATOM | 326 | CA | GLU | A | 56 | 55.996 | 55.810 | 85.736 | 1.00 | 51.46 | C |
| ATOM | 327 | CB | GLU | A | 56 | 55.878 | 56.455 | 84.349 | 1.00 | 51.02 | C |
| ATOM | 328 | CG | GLU | A | 56 | 54.495 | 56.342 | 83.722 | 1.00 | 50.90 | C |
| ATOM | 329 | CD | GLU | A | 56 | 53.399 | 56.816 | 84.656 | 1.00 | 51.07 | C |
| ATOM | 330 | OE1 | GLU | A | 56 | 53.647 | 57.754 | 85.445 | 1.00 | 52.45 | O |
| ATOM | 331 | OE2 | GLU | A | 56 | 52.284 | 56.262 | 84.598 | 1.00 | 50.91 | O |
| ATOM | 332 | C | GLU | A | 56 | 55.871 | 54.283 | 85.635 | 1.00 | 49.14 | C |
| ATOM | 333 | O | GLU | A | 56 | 54.819 | 53.731 | 85.957 | 1.00 | 46.92 | O |
| ATOM | 334 | N | LEU | A | 57 | 56.926 | 53.599 | 85.197 | 1.00 | 48.13 | N |
| ATOM | 335 | CA | LEU | A | 57 | 56.863 | 52.141 | 85.086 | 1.00 | 47.59 | C |
| ATOM | 336 | CB | LEU | A | 57 | 58.177 | 51.587 | 84.522 | 1.00 | 47.09 | C |
| ATOM | 337 | CG | LEU | A | 57 | 58.324 | 51.862 | 83.022 | 1.00 | 47.45 | C |
| ATOM | 338 | CD1 | LEU | A | 57 | 59.740 | 51.571 | 82.559 | 1.00 | 47.67 | C |
| ATOM | 339 | CD2 | LEU | A | 57 | 57.308 | 51.008 | 82.251 | 1.00 | 47.92 | C |
| ATOM | 340 | C | LEU | A | 57 | 56.537 | 51.526 | 86.446 | 1.00 | 46.84 | C |
| ATOM | 341 | O | LEU | A | 57 | 55.664 | 50.671 | 86.550 | 1.00 | 47.38 | O |
| ATOM | 342 | N | PRO | A | 58 | 57.232 | 51.960 | 87.509 | 1.00 | 47.38 | N |
| ATOM | 343 | CD | PRO | A | 58 | 58.492 | 52.731 | 87.488 | 1.00 | 47.39 | C |
| ATOM | 344 | CA | PRO | A | 58 | 56.975 | 51.432 | 88.852 | 1.00 | 47.32 | C |
| ATOM | 345 | CB | PRO | A | 58 | 57.993 | 52.181 | 89.707 | 1.00 | 47.78 | C |
| ATOM | 346 | CG | PRO | A | 58 | 59.162 | 52.296 | 88.780 | 1.00 | 47.75 | C |
| ATOM | 347 | C | PRO | A | 58 | 55.530 | 51.688 | 89.297 | 1.00 | 48.13 | C |
| ATOM | 348 | O | PRO | A | 58 | 54.930 | 50.877 | 90.020 | 1.00 | 48.44 | O |
| ATOM | 349 | N | VAL | A | 59 | 54.971 | 52.819 | 88.873 | 1.00 | 47.47 | N |
| ATOM | 350 | CA | VAL | A | 59 | 53.590 | 53.142 | 89.224 | 1.00 | 47.01 | C |
| ATOM | 351 | CB | VAL | A | 59 | 53.235 | 54.613 | 88.869 | 1.00 | 47.82 | C |
| ATOM | 352 | CG1 | VAL | A | 59 | 51.748 | 54.874 | 89.145 | 1.00 | 47.50 | C |
| ATOM | 353 | CG2 | VAL | A | 59 | 54.105 | 55.570 | 89.682 | 1.00 | 47.75 | C |
| ATOM | 354 | C | VAL | A | 59 | 52.627 | 52.211 | 88.482 | 1.00 | 46.54 | C |
| ATOM | 355 | O | VAL | A | 59 | 51.642 | 51.734 | 89.048 | 1.00 | 46.60 | O |
| ATOM | 356 | N | ARG | A | 60 | 52.915 | 51.948 | 87.213 | 1.00 | 45.48 | N |
| ATOM | 357 | CA | ARG | A | 60 | 52.043 | 51.079 | 86.434 | 1.00 | 45.85 | C |
| ATOM | 358 | CB | ARG | A | 60 | 52.326 | 51.241 | 84.943 | 1.00 | 43.66 | C |
| ATOM | 359 | CG | ARG | A | 60 | 52.022 | 52.649 | 84.452 | 1.00 | 42.89 | C |
| ATOM | 360 | CD | ARG | A | 60 | 51.940 | 52.730 | 82.947 | 1.00 | 41.21 | C |
| ATOM | 361 | NE | ARG | A | 60 | 51.781 | 54.107 | 82.517 | 1.00 | 39.94 | N |
| ATOM | 362 | CZ | ARG | A | 60 | 51.530 | 54.479 | 81.268 | 1.00 | 39.82 | C |
| ATOM | 363 | NH1 | ARG | A | 60 | 51.396 | 53.567 | 80.314 | 1.00 | 38.90 | N |
| ATOM | 364 | NH2 | ARG | A | 60 | 51.439 | 55.767 | 80.972 | 1.00 | 38.96 | N |
| ATOM | 365 | C | ARG | A | 60 | 52.178 | 49.623 | 86.860 | 1.00 | 46.19 | C |
| ATOM | 366 | O | ARG | A | 60 | 51.173 | 48.924 | 87.006 | 1.00 | 45.54 | O |
| ATOM | 367 | N | LEU | A | 61 | 53.414 | 49.176 | 87.082 | 1.00 | 46.29 | N |
| ATOM | 368 | CA | LEU | A | 61 | 53.656 | 47.804 | 87.523 | 1.00 | 46.70 | C |
| ATOM | 369 | CB | LEU | A | 61 | 55.153 | 47.572 | 87.705 | 1.00 | 46.71 | C |
| ATOM | 370 | CG | LEU | A | 61 | 55.924 | 47.571 | 86.393 | 1.00 | 46.26 | C |
| ATOM | 371 | CD1 | LEU | A | 61 | 57.413 | 47.563 | 86.691 | 1.00 | 45.71 | C |
| ATOM | 372 | CD2 | LEU | A | 61 | 55.502 | 46.359 | 85.559 | 1.00 | 46.47 | C |
| ATOM | 373 | C | LEU | A | 61 | 52.939 | 47.547 | 88.845 | 1.00 | 46.71 | C |
| ATOM | 374 | O | LEU | A | 61 | 52.199 | 46.572 | 88.990 | 1.00 | 46.19 | O |
| ATOM | 375 | N | ALA | A | 62 | 53.152 | 48.450 | 89.795 | 1.00 | 47.78 | N |
| ATOM | 376 | CA | ALA | A | 62 | 52.552 | 48.348 | 91.118 | 1.00 | 48.88 | C |
| ATOM | 377 | CB | ALA | A | 62 | 53.069 | 49.480 | 92.014 | 1.00 | 49.15 | C |
| ATOM | 378 | C | ALA | A | 62 | 51.027 | 48.354 | 91.097 | 1.00 | 49.40 | C |
| ATOM | 379 | O | ALA | A | 62 | 50.396 | 47.624 | 91.869 | 1.00 | 49.35 | O |
| ATOM | 380 | N | ASN | A | 63 | 50.427 | 49.174 | 90.234 | 1.00 | 50.61 | N |
| ATOM | 381 | CA | ASN | A | 63 | 48.968 | 49.228 | 90.172 | 1.00 | 51.33 | C |
| ATOM | 382 | CB | ASN | A | 63 | 48.478 | 50.397 | 89.294 | 1.00 | 52.55 | C |
| ATOM | 383 | CG | ASN | A | 63 | 48.306 | 51.694 | 90.092 | 1.00 | 54.37 | C |
| ATOM | 384 | OD1 | ASN | A | 63 | 49.264 | 52.433 | 90.324 | 1.00 | 54.72 | O |
| ATOM | 385 | ND2 | ASN | A | 63 | 47.083 | 51.957 | 90.535 | 1.00 | 55.17 | N |
| ATOM | 386 | C | ASN | A | 63 | 48.374 | 47.916 | 89.684 | 1.00 | 51.13 | C |
| ATOM | 387 | O | ASN | A | 63 | 47.389 | 47.431 | 90.238 | 1.00 | 50.42 | O |
| ATOM | 388 | N | ILE | A | 64 | 48.964 | 47.319 | 88.657 | 1.00 | 52.06 | N |
| ATOM | 389 | CA | ILE | A | 64 | 48.403 | 46.064 | 88.198 | 1.00 | 53.22 | C |
| ATOM | 390 | CB | ILE | A | 64 | 48.842 | 45.717 | 86.761 | 1.00 | 52.01 | C |
| ATOM | 391 | CG2 | ILE | A | 64 | 50.272 | 45.231 | 86.725 | 1.00 | 52.66 | C |
| ATOM | 392 | CG1 | ILE | A | 64 | 47.906 | 44.638 | 86.224 | 1.00 | 52.78 | C |
| ATOM | 393 | CD1 | ILE | A | 64 | 46.428 | 44.982 | 86.381 | 1.00 | 50.71 | C |
| ATOM | 394 | C | ILE | A | 64 | 48.745 | 44.926 | 89.169 | 1.00 | 54.64 | C |
| ATOM | 395 | O | ILE | A | 64 | 47.998 | 43.951 | 89.290 | 1.00 | 54.16 | O |
| ATOM | 396 | N | MET | A | 65 | 49.858 | 45.064 | 89.881 | 1.00 | 55.40 | N |
| ATOM | 397 | CA | MET | A | 65 | 50.250 | 44.046 | 90.846 | 1.00 | 57.56 | C |
| ATOM | 398 | CB | MET | A | 65 | 51.705 | 44.218 | 91.253 | 1.00 | 56.49 | C |
| ATOM | 399 | CG | MET | A | 65 | 52.680 | 43.839 | 90.184 | 1.00 | 55.64 | C |
| ATOM | 400 | SD | MET | A | 65 | 54.278 | 44.437 | 90.629 | 1.00 | 55.60 | S |
| ATOM | 401 | CE | MET | A | 65 | 54.727 | 43.219 | 91.894 | 1.00 | 54.74 | C |
| ATOM | 402 | C | MET | A | 65 | 49.391 | 44.135 | 92.092 | 1.00 | 59.46 | C |
| ATOM | 403 | O | MET | A | 65 | 49.119 | 43.125 | 92.747 | 1.00 | 59.20 | O |
| ATOM | 404 | N | LYS | A | 66 | 48.979 | 45.355 | 92.421 | 1.00 | 61.72 | N |
| ATOM | 405 | CA | LYS | A | 66 | 48.152 | 45.594 | 93.591 | 1.00 | 64.08 | C |
| ATOM | 406 | CB | LYS | A | 66 | 47.967 | 47.102 | 93.801 | 1.00 | 64.54 | C |
| ATOM | 407 | CG | LYS | A | 66 | 48.016 | 47.560 | 95.251 | 1.00 | 66.03 | C |
| ATOM | 408 | CD | LYS | A | 66 | 46.835 | 47.071 | 96.065 | 1.00 | 66.84 | C |
| ATOM | 409 | CE | LYS | A | 66 | 46.929 | 47.570 | 97.509 | 1.00 | 68.36 | C |
| ATOM | 410 | NZ | LYS | A | 66 | 45.770 | 47.123 | 98.343 | 1.00 | 69.45 | N |
| ATOM | 411 | C | LYS | A | 66 | 46.805 | 44.928 | 93.344 | 1.00 | 65.46 | C |
| ATOM | 412 | O | LYS | A | 66 | 46.235 | 44.291 | 94.225 | 1.00 | 66.00 | O |
| ATOM | 413 | N | GLU | A | 67 | 46.309 | 45.076 | 92.125 | 1.00 | 67.15 | N |
| ATOM | 414 | CA | GLU | A | 67 | 45.029 | 44.509 | 91.730 | 1.00 | 69.40 | C |
| ATOM | 415 | CB | GLU | A | 67 | 44.713 | 44.936 | 90.302 | 1.00 | 71.12 | C |
| ATOM | 416 | CG | GLU | A | 67 | 43.355 | 44.528 | 89.816 | 1.00 | 73.59 | C |
| ATOM | 417 | CD | GLU | A | 67 | 42.849 | 45.467 | 88.755 | 1.00 | 75.63 | C |
| ATOM | 418 | OE1 | GLU | A | 67 | 43.600 | 46.395 | 88.378 | 1.00 | 77.06 | O |
| ATOM | 419 | OE2 | GLU | A | 67 | 41.703 | 45.283 | 88.298 | 1.00 | 76.88 | O |
| ATOM | 420 | C | GLU | A | 67 | 45.057 | 42.988 | 91.821 | 1.00 | 70.00 | C |
| ATOM | 421 | O | GLU | A | 67 | 44.139 | 42.356 | 92.359 | 1.00 | 70.43 | O |
| ATOM | 422 | N | ILE | A | 68 | 46.122 | 42.414 | 91.276 | 1.00 | 69.94 | N |
| ATOM | 423 | CA | ILE | A | 68 | 46.335 | 40.977 | 91.283 | 1.00 | 69.76 | C |
| ATOM | 424 | CB | ILE | A | 68 | 47.708 | 40.649 | 90.644 | 1.00 | 68.84 | C |
| ATOM | 425 | CG2 | ILE | A | 68 | 48.184 | 39.283 | 91.063 | 1.00 | 68.28 | C |
| ATOM | 426 | CG1 | ILE | A | 68 | 47.604 | 40.731 | 89.124 | 1.00 | 67.94 | C |
| ATOM | 427 | CD1 | ILE | A | 68 | 48.918 | 40.537 | 88.420 | 1.00 | 67.17 | C |
| ATOM | 428 | C | ILE | A | 68 | 46.268 | 40.408 | 92.704 | 1.00 | 70.76 | C |
| ATOM | 429 | O | ILE | A | 68 | 45.537 | 39.448 | 92.960 | 1.00 | 70.55 | O |
| ATOM | 430 | N | ASN | A | 69 | 47.021 | 41.013 | 93.621 | 1.00 | 71.70 | N |
| ATOM | 431 | CA | ASN | A | 69 | 47.066 | 40.573 | 95.015 | 1.00 | 73.07 | C |
| ATOM | 432 | CB | ASN | A | 69 | 48.016 | 41.452 | 95.833 | 1.00 | 73.93 | C |
| ATOM | 433 | CG | ASN | A | 69 | 49.469 | 41.206 | 95.501 | 1.00 | 74.84 | C |
| ATOM | 434 | OD1 | ASN | A | 69 | 49.873 | 40.072 | 95.262 | 1.00 | 75.71 | O |
| ATOM | 435 | ND2 | ASN | A | 69 | 50.269 | 42.267 | 95.503 | 1.00 | 75.35 | N |
| ATOM | 436 | C | ASN | A | 69 | 45.732 | 40.530 | 95.740 | 1.00 | 73.47 | C |
| ATOM | 437 | O | ASN | A | 69 | 45.613 | 39.872 | 96.773 | 1.00 | 74.25 | O |
| ATOM | 438 | N | LEU | A | 70 | 44.733 | 41.231 | 95.222 | 1.00 | 73.50 | N |
| ATOM | 439 | CA | LEU | A | 70 | 43.434 | 41.245 | 95.876 | 1.00 | 74.08 | C |
| ATOM | 440 | CB | LEU | A | 70 | 42.884 | 42.677 | 95.893 | 1.00 | 75.52 | C |
| ATOM | 441 | CG | LEU | A | 70 | 43.764 | 43.609 | 96.749 | 1.00 | 76.39 | C |
| ATOM | 442 | CD1 | LEU | A | 70 | 43.188 | 45.019 | 96.775 | 1.00 | 76.43 | C |
| ATOM | 443 | CD2 | LEU | A | 70 | 43.871 | 43.043 | 98.169 | 1.00 | 75.94 | C |
| ATOM | 444 | C | LEU | A | 70 | 42.457 | 40.260 | 95.237 | 1.00 | 74.01 | C |
| ATOM | 445 | O | LEU | A | 70 | 41.253 | 40.288 | 95.488 | 1.00 | 73.71 | O |
| ATOM | 446 | N | LEU | A | 71 | 43.015 | 39.370 | 94.421 | 1.00 | 73.87 | N |
| ATOM | 447 | CA | LEU | A | 71 | 42.268 | 38.320 | 93.728 | 1.00 | 73.39 | C |
| ATOM | 448 | CB | LEU | A | 71 | 43.108 | 37.772 | 92.579 | 1.00 | 72.85 | C |
| ATOM | 449 | CG | LEU | A | 71 | 43.059 | 38.373 | 91.182 | 1.00 | 72.47 | C |
| ATOM | 450 | CD1 | LEU | A | 71 | 44.328 | 38.007 | 90.424 | 1.00 | 71.74 | C |
| ATOM | 451 | CD2 | LEU | A | 71 | 41.825 | 37.850 | 90.467 | 1.00 | 72.06 | C |
| ATOM | 452 | C | LEU | A | 71 | 41.981 | 37.161 | 94.679 | 1.00 | 73.25 | C |
| ATOM | 453 | O | LEU | A | 71 | 42.764 | 36.907 | 95.595 | 1.00 | 72.85 | O |
| ATOM | 454 | N | PRO | A | 72 | 40.866 | 36.438 | 94.476 | 1.00 | 73.36 | N |
| ATOM | 455 | CD | PRO | A | 72 | 39.763 | 36.587 | 93.507 | 1.00 | 73.15 | C |
| ATOM | 456 | CA | PRO | A | 72 | 40.613 | 35.322 | 95.392 | 1.00 | 73.87 | C |
| ATOM | 457 | CB | PRO | A | 72 | 39.464 | 34.584 | 94.720 | 1.00 | 73.94 | C |
| ATOM | 458 | CG | PRO | A | 72 | 38.682 | 35.697 | 94.104 | 1.00 | 73.50 | C |
| ATOM | 459 | C | PRO | A | 72 | 41.884 | 34.472 | 95.461 | 1.00 | 74.37 | C |
| ATOM | 460 | O | PRO | A | 72 | 42.601 | 34.346 | 94.467 | 1.00 | 74.21 | O |
| ATOM | 461 | N | ASP | A | 73 | 42.172 | 33.907 | 96.629 | 1.00 | 75.26 | N |
| ATOM | 462 | CA | ASP | A | 73 | 43.366 | 33.086 | 96.791 | 1.00 | 75.83 | C |
| ATOM | 463 | CB | ASP | A | 73 | 43.549 | 32.682 | 98.259 | 1.00 | 77.55 | C |
| ATOM | 464 | CG | ASP | A | 73 | 44.849 | 31.922 | 98.498 | 1.00 | 79.04 | C |
| ATOM | 465 | OD1 | ASP | A | 73 | 45.935 | 32.486 | 98.224 | 1.00 | 79.99 | O |
| ATOM | 466 | OD2 | ASP | A | 73 | 44.785 | 30.762 | 98.963 | 1.00 | 79.22 | O |
| ATOM | 467 | C | ASP | A | 73 | 43.271 | 31.842 | 95.918 | 1.00 | 75.17 | C |
| ATOM | 468 | O | ASP | A | 73 | 44.286 | 31.222 | 95.597 | 1.00 | 75.28 | O |
| ATOM | 469 | N | ARG | A | 74 | 42.052 | 31.484 | 95.523 | 1.00 | 74.54 | N |
| ATOM | 470 | CA | ARG | A | 74 | 41.860 | 30.311 | 94.680 | 1.00 | 74.31 | C |
| ATOM | 471 | CB | ARG | A | 74 | 40.390 | 29.857 | 94.705 | 1.00 | 74.45 | C |
| ATOM | 472 | CG | ARG | A | 74 | 39.461 | 30.541 | 93.725 | 1.00 | 75.11 | C |
| ATOM | 473 | CD | ARG | A | 74 | 38.021 | 30.135 | 93.990 | 1.00 | 75.90 | C |
| ATOM | 474 | NE | ARG | A | 74 | 37.070 | 30.943 | 93.230 | 1.00 | 77.07 | N |
| ATOM | 475 | CZ | ARG | A | 74 | 36.309 | 31.899 | 93.755 | 1.00 | 77.48 | C |
| ATOM | 476 | NH1 | ARG | A | 74 | 36.382 | 32.170 | 95.052 | 1.00 | 77.89 | N |
| ATOM | 477 | NH2 | ARG | A | 74 | 35.474 | 32.582 | 92.984 | 1.00 | 77.07 | N |
| ATOM | 478 | C | ARG | A | 74 | 42.331 | 30.592 | 93.244 | 1.00 | 73.74 | C |
| ATOM | 479 | O | ARG | A | 74 | 42.482 | 29.674 | 92.438 | 1.00 | 73.39 | O |
| ATOM | 480 | N | VAL | A | 75 | 42.562 | 31.865 | 92.932 | 1.00 | 72.97 | N |
| ATOM | 481 | CA | VAL | A | 75 | 43.060 | 32.261 | 91.618 | 1.00 | 71.77 | C |
| ATOM | 482 | CB | VAL | A | 75 | 42.407 | 33.563 | 91.114 | 1.00 | 71.76 | C |
| ATOM | 483 | CG1 | VAL | A | 75 | 42.996 | 33.952 | 89.763 | 1.00 | 71.65 | C |
| ATOM | 484 | CG2 | VAL | A | 75 | 40.911 | 33.380 | 90.998 | 1.00 | 71.59 | C |
| ATOM | 485 | C | VAL | A | 75 | 44.536 | 32.536 | 91.840 | 1.00 | 71.34 | C |
| ATOM | 486 | O | VAL | A | 75 | 45.394 | 32.142 | 91.052 | 1.00 | 71.33 | O |
| ATOM | 487 | N | LEU | A | 76 | 44.803 | 33.218 | 92.945 | 1.00 | 70.68 | N |
| ATOM | 488 | CA | LEU | A | 76 | 46.145 | 33.593 | 93.353 | 1.00 | 70.69 | C |
| ATOM | 489 | CB | LEU | A | 76 | 46.077 | 34.322 | 94.688 | 1.00 | 70.66 | C |
| ATOM | 490 | CG | LEU | A | 76 | 46.377 | 35.811 | 94.668 | 1.00 | 70.61 | C |
| ATOM | 491 | CD1 | LEU | A | 76 | 46.118 | 36.382 | 96.047 | 1.00 | 70.86 | C |
| ATOM | 492 | CD2 | LEU | A | 76 | 47.822 | 36.038 | 94.258 | 1.00 | 70.47 | C |
| ATOM | 493 | C | LEU | A | 76 | 47.127 | 32.432 | 93.478 | 1.00 | 70.21 | C |
| ATOM | 494 | O | LEU | A | 76 | 48.257 | 32.523 | 93.003 | 1.00 | 70.48 | O |
| ATOM | 495 | N | SER | A | 77 | 46.691 | 31.347 | 94.113 | 1.00 | 69.53 | N |
| ATOM | 496 | CA | SER | A | 77 | 47.540 | 30.181 | 94.337 | 1.00 | 68.95 | C |
| ATOM | 497 | CB | SER | A | 77 | 46.882 | 29.251 | 95.357 | 1.00 | 69.34 | C |
| ATOM | 498 | OG | SER | A | 77 | 46.923 | 29.828 | 96.652 | 1.00 | 70.22 | O |
| ATOM | 499 | C | SER | A | 77 | 47.955 | 29.361 | 93.122 | 1.00 | 68.33 | C |
| ATOM | 500 | O | SER | A | 77 | 48.883 | 28.547 | 93.225 | 1.00 | 68.43 | O |
| ATOM | 501 | N | THR | A | 78 | 47.288 | 29.558 | 91.986 | 1.00 | 66.64 | N |
| ATOM | 502 | CA | THR | A | 78 | 47.624 | 28.797 | 90.792 | 1.00 | 65.36 | C |
| ATOM | 503 | CB | THR | A | 78 | 46.633 | 29.087 | 89.632 | 1.00 | 65.26 | C |
| ATOM | 504 | OG1 | THR | A | 78 | 46.773 | 30.448 | 89.208 | 1.00 | 64.48 | O |
| ATOM | 505 | CG2 | THR | A | 78 | 45.204 | 28.832 | 90.084 | 1.00 | 63.80 | C |
| ATOM | 506 | C | THR | A | 78 | 49.029 | 29.129 | 90.301 | 1.00 | 64.59 | C |
| ATOM | 507 | O | THR | A | 78 | 49.457 | 30.279 | 90.381 | 1.00 | 64.73 | O |
| ATOM | 508 | N | PRO | A | 79 | 49.771 | 28.120 | 89.816 | 1.00 | 63.77 | N |
| ATOM | 509 | CD | PRO | A | 79 | 49.365 | 26.717 | 89.628 | 1.00 | 62.80 | C |
| ATOM | 510 | CA | PRO | A | 79 | 51.131 | 28.343 | 89.314 | 1.00 | 62.93 | C |
| ATOM | 511 | CB | PRO | A | 79 | 51.498 | 26.975 | 88.714 | 1.00 | 62.65 | C |
| ATOM | 512 | CG | PRO | A | 79 | 50.691 | 26.012 | 89.541 | 1.00 | 62.28 | C |
| ATOM | 513 | C | PRO | A | 79 | 51.077 | 29.454 | 88.276 | 1.00 | 61.95 | C |
| ATOM | 514 | O | PRO | A | 79 | 52.040 | 30.170 | 88.039 | 1.00 | 61.78 | O |
| ATOM | 515 | N | SER | A | 80 | 49.899 | 29.566 | 87.687 | 1.00 | 61.00 | N |
| ATOM | 516 | CA | SER | A | 80 | 49.579 | 30.540 | 86.637 | 1.00 | 60.19 | C |
| ATOM | 517 | CB | SER | A | 80 | 48.079 | 30.455 | 86.347 | 1.00 | 60.60 | C |
| ATOM | 518 | OG | SER | A | 80 | 47.704 | 29.129 | 86.294 | 1.00 | 65.23 | O |
| ATOM | 519 | C | SER | A | 80 | 49.884 | 31.942 | 87.080 | 1.00 | 58.50 | C |
| ATOM | 520 | O | SER | A | 80 | 50.918 | 32.542 | 86.753 | 1.00 | 56.48 | O |
| ATOM | 521 | N | VAL | A | 81 | 48.949 | 32.467 | 87.858 | 1.00 | 58.17 | N |
| ATOM | 522 | CA | VAL | A | 81 | 49.067 | 33.812 | 88.396 | 1.00 | 58.07 | C |
| ATOM | 523 | CB | VAL | A | 81 | 47.807 | 34.015 | 89.161 | 1.00 | 57.88 | C |
| ATOM | 524 | CG1 | VAL | A | 81 | 47.879 | 35.403 | 89.816 | 1.00 | 57.90 | C |
| ATOM | 525 | CG2 | VAL | A | 81 | 46.617 | 33.853 | 88.271 | 1.00 | 56.20 | C |
| ATOM | 526 | C | VAL | A | 81 | 50.355 | 34.006 | 89.167 | 1.00 | 57.93 | C |
| ATOM | 527 | O | VAL | A | 81 | 50.928 | 35.096 | 89.125 | 1.00 | 57.54 | O |
| ATOM | 528 | N | GLN | A | 82 | 50.767 | 32.987 | 89.913 | 1.00 | 58.00 | N |
| ATOM | 529 | CA | GLN | A | 82 | 51.961 | 33.155 | 90.736 | 1.00 | 57.95 | C |
| ATOM | 530 | CB | GLN | A | 82 | 52.120 | 31.969 | 91.696 | 1.00 | 60.38 | C |
| ATOM | 531 | CG | GLN | A | 82 | 52.710 | 32.356 | 93.066 | 1.00 | 64.50 | C |
| ATOM | 532 | CD | GLN | A | 82 | 51.745 | 33.192 | 93.902 | 1.00 | 67.14 | C |
| ATOM | 533 | OE1 | GLN | A | 82 | 50.533 | 33.157 | 93.686 | 1.00 | 68.16 | O |
| ATOM | 534 | NE2 | GLN | A | 82 | 52.278 | 33.932 | 94.873 | 1.00 | 68.81 | N |
| ATOM | 535 | C | GLN | A | 82 | 53.239 | 33.323 | 89.919 | 1.00 | 56.00 | C |
| ATOM | 536 | O | GLN | A | 82 | 54.152 | 34.040 | 90.322 | 1.00 | 55.45 | O |
| ATOM | 537 | N | LEU | A | 83 | 53.316 | 32.655 | 88.775 | 1.00 | 54.52 | N |
| ATOM | 538 | CA | LEU | A | 83 | 54.491 | 32.760 | 87.922 | 1.00 | 54.32 | C |
| ATOM | 539 | CB | LEU | A | 83 | 54.420 | 31.697 | 86.828 | 1.00 | 53.95 | C |
| ATOM | 540 | CG | LEU | A | 83 | 55.471 | 31.776 | 85.727 | 1.00 | 55.28 | C |
| ATOM | 541 | CD1 | LEU | A | 83 | 56.854 | 32.000 | 86.310 | 1.00 | 55.35 | C |
| ATOM | 542 | CD2 | LEU | A | 83 | 55.427 | 30.492 | 84.934 | 1.00 | 57.06 | C |
| ATOM | 543 | C | LEU | A | 83 | 54.577 | 34.157 | 87.300 | 1.00 | 54.22 | C |
| ATOM | 544 | O | LEU | A | 83 | 55.643 | 34.783 | 87.279 | 1.00 | 53.59 | O |
| ATOM | 545 | N | VAL | A | 84 | 53.439 | 34.626 | 86.792 | 1.00 | 53.88 | N |
| ATOM | 546 | CA | VAL | A | 84 | 53.336 | 35.938 | 86.171 | 1.00 | 54.07 | C |
| ATOM | 547 | CB | VAL | A | 84 | 51.935 | 36.147 | 85.575 | 1.00 | 53.65 | C |
| ATOM | 548 | CG1 | VAL | A | 84 | 51.714 | 37.620 | 85.258 | 1.00 | 52.59 | C |
| ATOM | 549 | CG2 | VAL | A | 84 | 51.782 | 35.295 | 84.326 | 1.00 | 52.13 | C |
| ATOM | 550 | C | VAL | A | 84 | 53.583 | 36.977 | 87.248 | 1.00 | 54.38 | C |
| ATOM | 551 | O | VAL | A | 84 | 54.327 | 37.936 | 87.056 | 1.00 | 53.57 | O |
| ATOM | 552 | N | GLN | A | 85 | 52.950 | 36.763 | 88.390 | 1.00 | 55.68 | N |
| ATOM | 553 | CA | GLN | A | 85 | 53.098 | 37.661 | 89.514 | 1.00 | 57.90 | C |
| ATOM | 554 | CB | GLN | A | 85 | 52.189 | 37.210 | 90.654 | 1.00 | 59.63 | C |
| ATOM | 555 | CG | GLN | A | 85 | 52.437 | 37.926 | 91.957 | 1.00 | 63.68 | C |
| ATOM | 556 | CD | GLN | A | 85 | 51.278 | 37.766 | 92.921 | 1.00 | 66.48 | C |
| ATOM | 557 | OE1 | GLN | A | 85 | 50.912 | 36.649 | 93.293 | 1.00 | 69.19 | O |
| ATOM | 558 | NE2 | GLN | A | 85 | 50.690 | 38.884 | 93.326 | 1.00 | 67.26 | N |
| ATOM | 559 | C | GLN | A | 85 | 54.543 | 37.724 | 89.992 | 1.00 | 57.79 | C |
| ATOM | 560 | O | GLN | A | 85 | 54.941 | 38.690 | 90.638 | 1.00 | 58.63 | O |
| ATOM | 561 | N | SER | A | 86 | 55.328 | 36.697 | 89.679 | 1.00 | 57.31 | N |
| ATOM | 562 | CA | SER | A | 86 | 56.720 | 36.685 | 90.098 | 1.00 | 57.06 | C |
| ATOM | 563 | CB | SER | A | 86 | 57.250 | 35.251 | 90.244 | 1.00 | 56.50 | C |
| ATOM | 564 | OG | SER | A | 86 | 57.554 | 34.667 | 88.989 | 1.00 | 55.82 | O |
| ATOM | 565 | C | SER | A | 86 | 57.546 | 37.453 | 89.075 | 1.00 | 57.52 | C |
| ATOM | 566 | O | SER | A | 86 | 58.536 | 38.092 | 89.430 | 1.00 | 57.62 | O |
| ATOM | 567 | N | TRP | A | 87 | 57.154 | 37.391 | 87.803 | 1.00 | 58.13 | N |
| ATOM | 568 | CA | TRP | A | 87 | 57.890 | 38.138 | 86.790 | 1.00 | 58.94 | C |
| ATOM | 569 | CB | TRP | A | 87 | 57.347 | 37.914 | 85.373 | 1.00 | 61.36 | C |
| ATOM | 570 | CG | TRP | A | 87 | 57.391 | 36.532 | 84.779 | 1.00 | 64.27 | C |
| ATOM | 571 | CD2 | TRP | A | 87 | 56.540 | 36.054 | 83.733 | 1.00 | 65.98 | C |
| ATOM | 572 | CE2 | TRP | A | 87 | 56.961 | 34.743 | 83.410 | 1.00 | 66.52 | C |
| ATOM | 573 | CE3 | TRP | A | 87 | 55.442 | 36.597 | 83.058 | 1.00 | 67.07 | C |
| ATOM | 574 | CD1 | TRP | A | 87 | 58.286 | 35.530 | 85.033 | 1.00 | 64.71 | C |
| ATOM | 575 | NE1 | TRP | A | 87 | 58.034 | 34.450 | 84.208 | 1.00 | 65.93 | N |
| ATOM | 576 | CZ2 | TRP | A | 87 | 56.339 | 33.984 | 82.406 | 1.00 | 66.80 | C |
| ATOM | 577 | CZ3 | TRP | A | 87 | 54.824 | 35.839 | 82.064 | 1.00 | 67.78 | C |
| ATOM | 578 | CH2 | TRP | A | 87 | 55.266 | 34.544 | 81.759 | 1.00 | 67.27 | C |
| ATOM | 579 | C | TRP | A | 87 | 57.733 | 39.632 | 87.111 | 1.00 | 57.99 | C |
| ATOM | 580 | O | TRP | A | 87 | 58.699 | 40.394 | 87.069 | 1.00 | 57.12 | O |
| ATOM | 581 | N | TYR | A | 88 | 56.510 | 40.053 | 87.421 | 1.00 | 57.40 | N |
| ATOM | 582 | CA | TYR | A | 88 | 56.273 | 41.458 | 87.735 | 1.00 | 58.23 | C |
| ATOM | 583 | CB | TYR | A | 88 | 54.778 | 41.745 | 87.939 | 1.00 | 57.92 | C |
| ATOM | 584 | CG | TYR | A | 88 | 54.014 | 41.875 | 86.643 | 1.00 | 58.95 | C |
| ATOM | 585 | CD1 | TYR | A | 88 | 53.773 | 40.760 | 85.843 | 1.00 | 58.43 | C |
| ATOM | 586 | CE1 | TYR | A | 88 | 53.141 | 40.881 | 84.610 | 1.00 | 59.04 | C |
| ATOM | 587 | CD2 | TYR | A | 88 | 53.593 | 43.124 | 86.177 | 1.00 | 59.59 | C |
| ATOM | 588 | CE2 | TYR | A | 88 | 52.957 | 43.254 | 84.935 | 1.00 | 59.40 | C |
| ATOM | 589 | CZ | TYR | A | 88 | 52.740 | 42.130 | 84.163 | 1.00 | 59.22 | C |
| ATOM | 590 | OH | TYR | A | 88 | 52.134 | 42.261 | 82.941 | 1.00 | 60.94 | O |
| ATOM | 591 | C | TYR | A | 88 | 57.052 | 41.902 | 88.959 | 1.00 | 58.50 | C |
| ATOM | 592 | O | TYR | A | 88 | 57.548 | 43.026 | 88.999 | 1.00 | 58.32 | O |
| ATOM | 593 | N | VAL | A | 89 | 57.165 | 41.030 | 89.958 | 1.00 | 58.60 | N |
| ATOM | 594 | CA | VAL | A | 89 | 57.912 | 41.385 | 91.158 | 1.00 | 58.34 | C |
| ATOM | 595 | CB | VAL | A | 89 | 57.886 | 40.258 | 92.216 | 1.00 | 58.42 | C |
| ATOM | 596 | CG1 | VAL | A | 89 | 58.926 | 40.542 | 93.295 | 1.00 | 58.03 | C |
| ATOM | 597 | CG2 | VAL | A | 89 | 56.492 | 40.152 | 92.851 | 1.00 | 57.82 | C |
| ATOM | 598 | C | VAL | A | 89 | 59.361 | 41.660 | 90.775 | 1.00 | 58.99 | C |
| ATOM | 599 | O | VAL | A | 89 | 59.960 | 42.636 | 91.225 | 1.00 | 58.62 | O |
| ATOM | 600 | N | GLN | A | 90 | 59.917 | 40.807 | 89.922 | 1.00 | 59.47 | N |
| ATOM | 601 | CA | GLN | A | 90 | 61.298 | 40.976 | 89.505 | 1.00 | 60.54 | C |
| ATOM | 602 | CB | GLN | A | 90 | 61.816 | 39.694 | 88.850 | 1.00 | 61.77 | C |
| ATOM | 603 | CG | GLN | A | 90 | 63.197 | 39.283 | 89.360 | 1.00 | 64.86 | C |
| ATOM | 604 | CD | GLN | A | 90 | 63.297 | 39.345 | 90.883 | 1.00 | 66.45 | C |
| ATOM | 605 | OE1 | GLN | A | 90 | 62.484 | 38.755 | 91.608 | 1.00 | 66.73 | O |
| ATOM | 606 | NE2 | GLN | A | 90 | 64.296 | 40.070 | 91.372 | 1.00 | 67.27 | N |
| ATOM | 607 | C | GLN | A | 90 | 61.502 | 42.162 | 88.571 | 1.00 | 60.49 | C |
| ATOM | 608 | O | GLN | A | 90 | 62.575 | 42.770 | 88.554 | 1.00 | 59.59 | O |
| ATOM | 609 | N | SER | A | 91 | 60.484 | 42.496 | 87.784 | 1.00 | 60.37 | N |
| ATOM | 610 | CA | SER | A | 91 | 60.619 | 43.632 | 86.881 | 1.00 | 59.90 | C |
| ATOM | 611 | CB | SER | A | 91 | 59.519 | 43.614 | 85.821 | 1.00 | 60.58 | C |
| ATOM | 612 | OG | SER | A | 91 | 59.788 | 42.608 | 84.858 | 1.00 | 60.53 | O |
| ATOM | 613 | C | SER | A | 91 | 60.576 | 44.915 | 87.706 | 1.00 | 59.38 | C |
| ATOM | 614 | O | SER | A | 91 | 61.319 | 45.855 | 87.440 | 1.00 | 57.93 | O |
| ATOM | 615 | N | LEU | A | 92 | 59.717 | 44.941 | 88.719 | 1.00 | 59.31 | N |
| ATOM | 616 | CA | LEU | A | 92 | 59.630 | 46.102 | 89.590 | 1.00 | 59.46 | C |
| ATOM | 617 | CB | LEU | A | 92 | 58.571 | 45.882 | 90.677 | 1.00 | 58.45 | C |
| ATOM | 618 | CG | LEU | A | 92 | 58.353 | 47.030 | 91.671 | 1.00 | 58.82 | C |
| ATOM | 619 | CD1 | LEU | A | 92 | 58.029 | 48.328 | 90.929 | 1.00 | 57.81 | C |
| ATOM | 620 | CD2 | LEU | A | 92 | 57.223 | 46.669 | 92.619 | 1.00 | 57.97 | C |
| ATOM | 621 | C | LEU | A | 92 | 61.008 | 46.323 | 90.227 | 1.00 | 60.58 | C |
| ATOM | 622 | O | LEU | A | 92 | 61.559 | 47.421 | 90.154 | 1.00 | 60.91 | O |
| ATOM | 623 | N | LEU | A | 93 | 61.570 | 45.273 | 90.831 | 1.00 | 61.20 | N |
| ATOM | 624 | CA | LEU | A | 93 | 62.886 | 45.356 | 91.473 | 1.00 | 62.13 | C |
| ATOM | 625 | CB | LEU | A | 93 | 63.225 | 44.029 | 92.158 | 1.00 | 61.54 | C |
| ATOM | 626 | CG | LEU | A | 93 | 62.209 | 43.537 | 93.193 | 1.00 | 61.41 | C |
| ATOM | 627 | CD1 | LEU | A | 93 | 62.597 | 42.161 | 93.693 | 1.00 | 60.57 | C |
| ATOM | 628 | CD2 | LEU | A | 93 | 62.127 | 44.524 | 94.343 | 1.00 | 61.70 | C |
| ATOM | 629 | C | LEU | A | 93 | 63.952 | 45.686 | 90.441 | 1.00 | 63.31 | C |
| ATOM | 630 | O | LEU | A | 93 | 64.890 | 46.434 | 90.710 | 1.00 | 63.09 | O |
| ATOM | 631 | N | ASP | A | 94 | 63.800 | 45.116 | 89.254 | 1.00 | 65.01 | N |
| ATOM | 632 | CA | ASP | A | 94 | 64.727 | 45.350 | 88.156 | 1.00 | 67.28 | C |
| ATOM | 633 | CB | ASP | A | 94 | 64.242 | 44.628 | 86.904 | 1.00 | 68.53 | C |
| ATOM | 634 | CG | ASP | A | 94 | 64.860 | 43.271 | 86.738 | 1.00 | 68.92 | C |
| ATOM | 635 | OD1 | ASP | A | 94 | 64.977 | 42.544 | 87.747 | 1.00 | 70.09 | O |
| ATOM | 636 | OD2 | ASP | A | 94 | 65.216 | 42.933 | 85.587 | 1.00 | 68.34 | O |
| ATOM | 637 | C | ASP | A | 94 | 64.868 | 46.830 | 87.822 | 1.00 | 68.72 | C |
| ATOM | 638 | O | ASP | A | 94 | 65.964 | 47.302 | 87.519 | 1.00 | 69.28 | O |
| ATOM | 639 | N | ILE | A | 95 | 63.760 | 47.563 | 87.870 | 1.00 | 70.00 | N |
| ATOM | 640 | CA | ILE | A | 95 | 63.792 | 48.977 | 87.517 | 1.00 | 71.37 | C |
| ATOM | 641 | CB | ILE | A | 95 | 62.471 | 49.425 | 86.830 | 1.00 | 71.80 | C |
| ATOM | 642 | CG2 | ILE | A | 95 | 62.628 | 50.825 | 86.246 | 1.00 | 72.01 | C |
| ATOM | 643 | CG1 | ILE | A | 95 | 62.136 | 48.488 | 85.677 | 1.00 | 71.97 | C |
| ATOM | 644 | CD1 | ILE | A | 95 | 60.885 | 48.869 | 84.938 | 1.00 | 73.38 | C |
| ATOM | 645 | C | ILE | A | 95 | 64.069 | 49.936 | 88.660 | 1.00 | 72.05 | C |
| ATOM | 646 | O | ILE | A | 95 | 64.296 | 51.115 | 88.434 | 1.00 | 72.15 | O |
| ATOM | 647 | N | MET | A | 96 | 64.053 | 49.456 | 89.890 | 1.00 | 73.09 | N |
| ATOM | 648 | CA | MET | A | 96 | 64.310 | 50.358 | 91.001 | 1.00 | 74.53 | C |
| ATOM | 649 | CB | MET | A | 96 | 63.846 | 49.708 | 92.291 | 1.00 | 75.16 | C |
| ATOM | 650 | CG | MET | A | 96 | 62.395 | 49.338 | 92.220 | 1.00 | 75.88 | C |
| ATOM | 651 | SD | MET | A | 96 | 61.332 | 50.507 | 93.026 | 1.00 | 76.81 | S |
| ATOM | 652 | CE | MET | A | 96 | 60.497 | 49.415 | 93.990 | 1.00 | 76.11 | C |
| ATOM | 653 | C | MET | A | 96 | 65.782 | 50.741 | 91.088 | 1.00 | 75.15 | C |
| ATOM | 654 | O | MET | A | 96 | 66.116 | 51.893 | 91.359 | 1.00 | 75.15 | O |
| ATOM | 655 | N | GLU | A | 97 | 66.653 | 49.772 | 90.838 | 1.00 | 76.00 | N |
| ATOM | 656 | CA | GLU | A | 97 | 68.096 | 49.981 | 90.879 | 1.00 | 77.27 | C |
| ATOM | 657 | CB | GLU | A | 97 | 68.798 | 48.944 | 89.994 | 1.00 | 78.32 | C |
| ATOM | 658 | CG | GLU | A | 97 | 68.389 | 47.492 | 90.246 | 1.00 | 79.95 | C |
| ATOM | 659 | CD | GLU | A | 97 | 68.991 | 46.913 | 91.512 | 1.00 | 80.54 | C |
| ATOM | 660 | OE1 | GLU | A | 97 | 70.223 | 46.702 | 91.545 | 1.00 | 81.33 | O |
| ATOM | 661 | OE2 | GLU | A | 97 | 68.234 | 46.669 | 92.476 | 1.00 | 81.18 | O |
| ATOM | 662 | C | GLU | A | 97 | 68.491 | 51.373 | 90.403 | 1.00 | 77.69 | C |
| ATOM | 663 | O | GLU | A | 97 | 69.536 | 51.888 | 90.788 | 1.00 | 78.08 | O |
| ATOM | 664 | N | PHE | A | 98 | 67.650 | 51.977 | 89.569 | 1.00 | 78.00 | N |
| ATOM | 665 | CA | PHE | A | 98 | 67.917 | 53.296 | 88.999 | 1.00 | 78.59 | C |
| ATOM | 666 | CB | PHE | A | 98 | 67.445 | 53.315 | 87.546 | 1.00 | 76.65 | C |
| ATOM | 667 | CG | PHE | A | 98 | 67.985 | 52.191 | 86.714 | 1.00 | 74.03 | C |
| ATOM | 668 | CD1 | PHE | A | 98 | 69.005 | 52.414 | 85.799 | 1.00 | 73.20 | C |
| ATOM | 669 | CD2 | PHE | A | 98 | 67.465 | 50.908 | 86.837 | 1.00 | 72.83 | C |
| ATOM | 670 | CE1 | PHE | A | 98 | 69.499 | 51.377 | 85.017 | 1.00 | 71.78 | C |
| ATOM | 671 | CE2 | PHE | A | 98 | 67.951 | 49.867 | 86.060 | 1.00 | 71.63 | C |
| ATOM | 672 | CZ | PHE | A | 98 | 68.969 | 50.102 | 85.148 | 1.00 | 71.61 | C |
| ATOM | 673 | C | PHE | A | 98 | 67.294 | 54.495 | 89.720 | 1.00 | 80.45 | C |
| ATOM | 674 | O | PHE | A | 98 | 67.488 | 55.636 | 89.296 | 1.00 | 80.12 | O |
| ATOM | 675 | N | LEU | A | 99 | 66.547 | 54.252 | 90.794 | 1.00 | 82.94 | N |
| ATOM | 676 | CA | LEU | A | 99 | 65.908 | 55.349 | 91.518 | 1.00 | 85.85 | C |
| ATOM | 677 | CB | LEU | A | 99 | 64.990 | 54.804 | 92.617 | 1.00 | 85.77 | C |
| ATOM | 678 | CG | LEU | A | 99 | 63.603 | 54.338 | 92.159 | 1.00 | 86.49 | C |
| ATOM | 679 | CD1 | LEU | A | 99 | 62.841 | 53.753 | 93.337 | 1.00 | 86.55 | C |
| ATOM | 680 | CD2 | LEU | A | 99 | 62.833 | 55.511 | 91.563 | 1.00 | 86.29 | C |
| ATOM | 681 | C | LEU | A | 99 | 66.873 | 56.371 | 92.117 | 1.00 | 87.64 | C |
| ATOM | 682 | O | LEU | A | 99 | 66.555 | 57.558 | 92.177 | 1.00 | 88.11 | O |
| ATOM | 683 | N | ASP | A | 100 | 68.044 | 55.919 | 92.557 | 1.00 | 89.48 | N |
| ATOM | 684 | CA | ASP | A | 100 | 69.023 | 56.831 | 93.141 | 1.00 | 91.37 | C |
| ATOM | 685 | CB | ASP | A | 100 | 69.628 | 56.240 | 94.420 | 1.00 | 91.59 | C |
| ATOM | 686 | CG | ASP | A | 100 | 70.273 | 54.880 | 94.193 | 1.00 | 92.32 | C |
| ATOM | 687 | OD1 | ASP | A | 100 | 70.905 | 54.678 | 93.136 | 1.00 | 92.21 | O |
| ATOM | 688 | OD2 | ASP | A | 100 | 70.158 | 54.016 | 95.085 | 1.00 | 92.66 | O |
| ATOM | 689 | C | ASP | A | 100 | 70.145 | 57.177 | 92.170 | 1.00 | 92.23 | C |
| ATOM | 690 | O | ASP | A | 100 | 70.978 | 58.038 | 92.459 | 1.00 | 92.45 | O |
| ATOM | 691 | N | LYS | A | 101 | 70.172 | 56.507 | 91.024 | 1.00 | 93.21 | N |
| ATOM | 692 | CA | LYS | A | 101 | 71.207 | 56.781 | 90.042 | 1.00 | 94.33 | C |
| ATOM | 693 | CB | LYS | A | 101 | 71.070 | 55.860 | 88.827 | 1.00 | 94.65 | C |
| ATOM | 694 | CG | LYS | A | 101 | 71.851 | 54.562 | 88.961 | 1.00 | 95.33 | C |
| ATOM | 695 | CD | LYS | A | 101 | 71.590 | 53.917 | 90.314 | 1.00 | 96.26 | C |
| ATOM | 696 | CE | LYS | A | 101 | 72.515 | 52.737 | 90.579 | 1.00 | 96.74 | C |
| ATOM | 697 | NZ | LYS | A | 101 | 72.358 | 52.233 | 91.977 | 1.00 | 97.26 | N |
| ATOM | 698 | C | LYS | A | 101 | 71.138 | 58.229 | 89.607 | 1.00 | 94.96 | C |
| ATOM | 699 | O | LYS | A | 101 | 70.336 | 59.013 | 90.117 | 1.00 | 95.42 | O |
| ATOM | 700 | N | ASP | A | 102 | 71.989 | 58.578 | 88.659 | 1.00 | 95.54 | N |
| ATOM | 701 | CA | ASP | A | 102 | 72.053 | 59.934 | 88.161 | 1.00 | 96.17 | C |
| ATOM | 702 | CB | ASP | A | 102 | 73.191 | 60.673 | 88.879 | 1.00 | 96.15 | C |
| ATOM | 703 | CG | ASP | A | 102 | 73.832 | 61.744 | 88.024 | 1.00 | 96.18 | C |
| ATOM | 704 | OD1 | ASP | A | 102 | 73.252 | 62.844 | 87.904 | 1.00 | 96.06 | O |
| ATOM | 705 | OD2 | ASP | A | 102 | 74.918 | 61.480 | 87.462 | 1.00 | 96.27 | O |
| ATOM | 706 | C | ASP | A | 102 | 72.319 | 59.868 | 86.672 | 1.00 | 96.58 | C |
| ATOM | 707 | O | ASP | A | 102 | 73.199 | 59.125 | 86.229 | 1.00 | 96.33 | O |
| ATOM | 708 | N | PRO | A | 103 | 71.535 | 60.605 | 85.869 | 1.00 | 97.05 | N |
| ATOM | 709 | CD | PRO | A | 103 | 70.280 | 61.321 | 86.155 | 1.00 | 97.04 | C |
| ATOM | 710 | CA | PRO | A | 103 | 71.791 | 60.559 | 84.429 | 1.00 | 97.48 | C |
| ATOM | 711 | CB | PRO | A | 103 | 70.708 | 61.466 | 83.856 | 1.00 | 97.32 | C |
| ATOM | 712 | CG | PRO | A | 103 | 69.578 | 61.275 | 84.817 | 1.00 | 97.20 | C |
| ATOM | 713 | C | PRO | A | 103 | 73.192 | 61.111 | 84.209 | 1.00 | 98.19 | C |
| ATOM | 714 | O | PRO | A | 103 | 74.100 | 60.823 | 84.988 | 1.00 | 98.54 | O |
| ATOM | 715 | N | GLU | A | 104 | 73.370 | 61.932 | 83.180 | 1.00 | 98.54 | N |
| ATOM | 716 | CA | GLU | A | 104 | 74.689 | 62.483 | 82.892 | 1.00 | 98.81 | C |
| ATOM | 717 | CB | GLU | A | 104 | 75.215 | 63.304 | 84.076 | 1.00 | 98.99 | C |
| ATOM | 718 | CG | GLU | A | 104 | 74.795 | 64.773 | 84.089 | 1.00 | 99.40 | C |
| ATOM | 719 | CD | GLU | A | 104 | 73.354 | 64.980 | 84.499 | 1.00 | 99.73 | C |
| ATOM | 720 | OE1 | GLU | A | 104 | 72.954 | 64.435 | 85.550 | 1.00 | 100.50 | O |
| ATOM | 721 | OE2 | GLU | A | 104 | 72.627 | 65.699 | 83.782 | 1.00 | 99.52 | O |
| ATOM | 722 | C | GLU | A | 104 | 75.657 | 61.340 | 82.580 | 1.00 | 98.91 | C |
| ATOM | 723 | O | GLU | A | 104 | 76.423 | 61.420 | 81.622 | 1.00 | 99.11 | O |
| ATOM | 724 | N | ASP | A | 105 | 75.624 | 60.283 | 83.390 | 1.00 | 99.02 | N |
| ATOM | 725 | CA | ASP | A | 105 | 76.483 | 59.125 | 83.172 | 1.00 | 99.20 | C |
| ATOM | 726 | CB | ASP | A | 105 | 76.501 | 58.215 | 84.407 | 1.00 | 99.56 | C |
| ATOM | 727 | CG | ASP | A | 105 | 77.511 | 57.078 | 84.282 | 1.00 | 100.10 | C |
| ATOM | 728 | OD1 | ASP | A | 105 | 78.726 | 57.359 | 84.222 | 1.00 | 100.65 | O |
| ATOM | 729 | OD2 | ASP | A | 105 | 77.093 | 55.903 | 84.240 | 1.00 | 100.66 | O |
| ATOM | 730 | C | ASP | A | 105 | 75.910 | 58.366 | 81.979 | 1.00 | 99.07 | C |
| ATOM | 731 | O | ASP | A | 105 | 74.932 | 57.629 | 82.109 | 1.00 | 98.80 | O |
| ATOM | 732 | N | HIS | A | 106 | 76.519 | 58.573 | 80.814 | 1.00 | 99.00 | N |
| ATOM | 733 | CA | HIS | A | 106 | 76.097 | 57.935 | 79.570 | 1.00 | 98.68 | C |
| ATOM | 734 | CB | HIS | A | 106 | 77.052 | 58.329 | 78.440 | 1.00 | 99.56 | C |
| ATOM | 735 | CG | HIS | A | 106 | 76.801 | 59.696 | 77.881 | 1.00 | 100.57 | C |
| ATOM | 736 | CD2 | HIS | A | 106 | 76.961 | 60.191 | 76.631 | 1.00 | 100.78 | C |
| ATOM | 737 | ND1 | HIS | A | 106 | 76.336 | 60.743 | 78.650 | 1.00 | 100.82 | N |
| ATOM | 738 | CE1 | HIS | A | 106 | 76.218 | 61.821 | 77.896 | 1.00 | 101.05 | C |
| ATOM | 739 | NE2 | HIS | A | 106 | 76.591 | 61.513 | 76.666 | 1.00 | 101.12 | N |
| ATOM | 740 | C | HIS | A | 106 | 76.044 | 56.417 | 79.690 | 1.00 | 98.09 | C |
| ATOM | 741 | O | HIS | A | 106 | 75.697 | 55.721 | 78.735 | 1.00 | 98.26 | O |
| ATOM | 742 | N | ARG | A | 107 | 76.379 | 55.915 | 80.874 | 1.00 | 97.00 | N |
| ATOM | 743 | CA | ARG | A | 107 | 76.391 | 54.483 | 81.137 | 1.00 | 95.72 | C |
| ATOM | 744 | CB | ARG | A | 107 | 77.745 | 54.108 | 81.746 | 1.00 | 96.33 | C |
| ATOM | 745 | CG | ARG | A | 107 | 78.174 | 52.668 | 81.554 | 1.00 | 97.18 | C |
| ATOM | 746 | CD | ARG | A | 107 | 79.671 | 52.539 | 81.813 | 1.00 | 98.24 | C |
| ATOM | 747 | NE | ARG | A | 107 | 80.122 | 51.150 | 81.827 | 1.00 | 98.94 | N |
| ATOM | 748 | CZ | ARG | A | 107 | 80.292 | 50.428 | 82.932 | 1.00 | 99.25 | C |
| ATOM | 749 | NH1 | ARG | A | 107 | 80.052 | 50.962 | 84.124 | 1.00 | 99.32 | N |
| ATOM | 750 | NH2 | ARG | A | 107 | 80.704 | 49.170 | 82.847 | 1.00 | 99.04 | N |
| ATOM | 751 | C | ARG | A | 107 | 75.242 | 54.103 | 82.081 | 1.00 | 94.34 | C |
| ATOM | 752 | O | ARG | A | 107 | 74.776 | 52.962 | 82.083 | 1.00 | 94.25 | O |
| ATOM | 753 | N | THR | A | 108 | 74.786 | 55.069 | 82.875 | 1.00 | 92.64 | N |
| ATOM | 754 | CA | THR | A | 108 | 73.687 | 54.848 | 83.810 | 1.00 | 90.80 | C |
| ATOM | 755 | CB | THR | A | 108 | 73.548 | 56.027 | 84.813 | 1.00 | 91.04 | C |
| ATOM | 756 | OG1 | THR | A | 108 | 74.611 | 55.974 | 85.774 | 1.00 | 91.68 | O |
| ATOM | 757 | CG2 | THR | A | 108 | 72.211 | 55.963 | 85.543 | 1.00 | 90.62 | C |
| ATOM | 758 | C | THR | A | 108 | 72.366 | 54.690 | 83.065 | 1.00 | 89.47 | C |
| ATOM | 759 | O | THR | A | 108 | 71.459 | 54.007 | 83.535 | 1.00 | 89.54 | O |
| ATOM | 760 | N | LEU | A | 109 | 72.261 | 55.315 | 81.897 | 1.00 | 87.60 | N |
| ATOM | 761 | CA | LEU | A | 109 | 71.033 | 55.243 | 81.120 | 1.00 | 85.70 | C |
| ATOM | 762 | CB | LEU | A | 109 | 70.690 | 56.627 | 80.557 | 1.00 | 85.32 | C |
| ATOM | 763 | CG | LEU | A | 109 | 71.374 | 57.137 | 79.291 | 1.00 | 85.00 | C |
| ATOM | 764 | CD1 | LEU | A | 109 | 71.118 | 58.631 | 79.182 | 1.00 | 84.45 | C |
| ATOM | 765 | CD2 | LEU | A | 109 | 72.865 | 56.848 | 79.328 | 1.00 | 85.85 | C |
| ATOM | 766 | C | LEU | A | 109 | 71.084 | 54.204 | 80.000 | 1.00 | 84.50 | C |
| ATOM | 767 | O | LEU | A | 109 | 70.083 | 53.962 | 79.325 | 1.00 | 84.61 | O |
| ATOM | 768 | N | SER | A | 110 | 72.245 | 53.589 | 79.800 | 1.00 | 82.70 | N |
| ATOM | 769 | CA | SER | A | 110 | 72.375 | 52.553 | 78.780 | 1.00 | 81.11 | C |
| ATOM | 770 | CB | SER | A | 110 | 73.765 | 52.581 | 78.143 | 1.00 | 81.07 | C |
| ATOM | 771 | OG | SER | A | 110 | 74.778 | 52.346 | 79.104 | 1.00 | 81.38 | O |
| ATOM | 772 | C | SER | A | 110 | 72.139 | 51.212 | 79.468 | 1.00 | 79.83 | C |
| ATOM | 773 | O | SER | A | 110 | 72.009 | 50.178 | 78.821 | 1.00 | 79.48 | O |
| ATOM | 774 | N | GLN | A | 111 | 72.097 | 51.248 | 80.794 | 1.00 | 78.70 | N |
| ATOM | 775 | CA | GLN | A | 111 | 71.855 | 50.059 | 81.591 | 1.00 | 77.33 | C |
| ATOM | 776 | CB | GLN | A | 111 | 72.597 | 50.146 | 82.917 | 1.00 | 78.07 | C |
| ATOM | 777 | CG | GLN | A | 111 | 74.066 | 49.844 | 82.790 | 1.00 | 79.28 | C |
| ATOM | 778 | CD | GLN | A | 111 | 74.879 | 50.409 | 83.936 | 1.00 | 80.49 | C |
| ATOM | 779 | OE1 | GLN | A | 111 | 76.079 | 50.151 | 84.039 | 1.00 | 80.99 | O |
| ATOM | 780 | NE2 | GLN | A | 111 | 74.235 | 51.192 | 84.798 | 1.00 | 80.58 | N |
| ATOM | 781 | C | GLN | A | 111 | 70.367 | 50.001 | 81.836 | 1.00 | 75.81 | C |
| ATOM | 782 | O | GLN | A | 111 | 69.834 | 48.968 | 82.239 | 1.00 | 75.86 | O |
| ATOM | 783 | N | PHE | A | 112 | 69.704 | 51.130 | 81.598 | 1.00 | 73.96 | N |
| ATOM | 784 | CA | PHE | A | 112 | 68.260 | 51.216 | 81.762 | 1.00 | 72.17 | C |
| ATOM | 785 | CB | PHE | A | 112 | 67.816 | 52.681 | 81.887 | 1.00 | 70.26 | C |
| ATOM | 786 | CG | PHE | A | 112 | 66.372 | 52.850 | 82.276 | 1.00 | 68.32 | C |
| ATOM | 787 | CD1 | PHE | A | 112 | 65.904 | 52.357 | 83.485 | 1.00 | 67.51 | C |
| ATOM | 788 | CD2 | PHE | A | 112 | 65.485 | 53.521 | 81.438 | 1.00 | 67.51 | C |
| ATOM | 789 | CE1 | PHE | A | 112 | 64.573 | 52.526 | 83.859 | 1.00 | 66.90 | C |
| ATOM | 790 | CE2 | PHE | A | 112 | 64.152 | 53.696 | 81.803 | 1.00 | 66.62 | C |
| ATOM | 791 | CZ | PHE | A | 112 | 63.697 | 53.198 | 83.016 | 1.00 | 66.65 | C |
| ATOM | 792 | C | PHE | A | 112 | 67.670 | 50.585 | 80.506 | 1.00 | 71.40 | C |
| ATOM | 793 | O | PHE | A | 112 | 66.799 | 49.718 | 80.588 | 1.00 | 71.52 | O |
| ATOM | 794 | N | THR | A | 113 | 68.171 | 51.010 | 79.347 | 1.00 | 70.05 | N |
| ATOM | 795 | CA | THR | A | 113 | 67.697 | 50.478 | 78.077 | 1.00 | 69.49 | C |
| ATOM | 796 | CB | THR | A | 113 | 68.490 | 51.045 | 76.892 | 1.00 | 69.44 | C |
| ATOM | 797 | OG1 | THR | A | 113 | 68.444 | 52.476 | 76.932 | 1.00 | 70.09 | O |
| ATOM | 798 | CG2 | THR | A | 113 | 67.892 | 50.563 | 75.581 | 1.00 | 68.79 | C |
| ATOM | 799 | C | THR | A | 113 | 67.825 | 48.959 | 78.066 | 1.00 | 68.93 | C |
| ATOM | 800 | O | THR | A | 113 | 66.909 | 48.258 | 77.638 | 1.00 | 68.85 | O |
| ATOM | 801 | N | ASP | A | 114 | 68.961 | 48.452 | 78.533 | 1.00 | 68.06 | N |
| ATOM | 802 | CA | ASP | A | 114 | 69.164 | 47.013 | 78.580 | 1.00 | 67.24 | C |
| ATOM | 803 | CB | ASP | A | 114 | 70.616 | 46.681 | 78.919 | 1.00 | 68.81 | C |
| ATOM | 804 | CG | ASP | A | 114 | 71.548 | 46.923 | 77.752 | 1.00 | 70.48 | C |
| ATOM | 805 | OD1 | ASP | A | 114 | 72.729 | 46.532 | 77.837 | 1.00 | 71.88 | O |
| ATOM | 806 | OD2 | ASP | A | 114 | 71.096 | 47.509 | 76.744 | 1.00 | 71.89 | O |
| ATOM | 807 | C | ASP | A | 114 | 68.235 | 46.408 | 79.616 | 1.00 | 65.62 | C |
| ATOM | 808 | O | ASP | A | 114 | 67.710 | 45.309 | 79.429 | 1.00 | 65.63 | O |
| ATOM | 809 | N | ALA | A | 115 | 68.029 | 47.137 | 80.706 | 1.00 | 63.46 | N |
| ATOM | 810 | CA | ALA | A | 115 | 67.148 | 46.685 | 81.769 | 1.00 | 62.35 | C |
| ATOM | 811 | CB | ALA | A | 115 | 67.209 | 47.655 | 82.936 | 1.00 | 62.36 | C |
| ATOM | 812 | C | ALA | A | 115 | 65.702 | 46.564 | 81.251 | 1.00 | 62.12 | C |
| ATOM | 813 | O | ALA | A | 115 | 64.928 | 45.723 | 81.723 | 1.00 | 61.60 | O |
| ATOM | 814 | N | LEU | A | 116 | 65.342 | 47.400 | 80.279 | 1.00 | 61.02 | N |
| ATOM | 815 | CA | LEU | A | 116 | 63.994 | 47.376 | 79.716 | 1.00 | 60.83 | C |
| ATOM | 816 | CB | LEU | A | 116 | 63.674 | 48.704 | 79.015 | 1.00 | 60.16 | C |
| ATOM | 817 | CG | LEU | A | 116 | 63.561 | 49.925 | 79.926 | 1.00 | 59.49 | C |
| ATOM | 818 | CD1 | LEU | A | 116 | 63.072 | 51.122 | 79.130 | 1.00 | 59.60 | C |
| ATOM | 819 | CD2 | LEU | A | 116 | 62.598 | 49.616 | 81.060 | 1.00 | 59.39 | C |
| ATOM | 820 | C | LEU | A | 116 | 63.813 | 46.221 | 78.740 | 1.00 | 60.43 | C |
| ATOM | 821 | O | LEU | A | 116 | 62.807 | 45.514 | 78.787 | 1.00 | 59.79 | O |
| ATOM | 822 | N | VAL | A | 117 | 64.789 | 46.031 | 77.859 | 1.00 | 59.81 | N |
| ATOM | 823 | CA | VAL | A | 117 | 64.727 | 44.949 | 76.883 | 1.00 | 59.25 | C |
| ATOM | 824 | CB | VAL | A | 117 | 65.985 | 44.917 | 75.986 | 1.00 | 58.74 | C |
| ATOM | 825 | CG1 | VAL | A | 117 | 65.927 | 43.719 | 75.060 | 1.00 | 58.52 | C |
| ATOM | 826 | CG2 | VAL | A | 117 | 66.087 | 46.204 | 75.174 | 1.00 | 58.60 | C |
| ATOM | 827 | C | VAL | A | 117 | 64.627 | 43.617 | 77.611 | 1.00 | 59.22 | C |
| ATOM | 828 | O | VAL | A | 117 | 63.943 | 42.699 | 77.157 | 1.00 | 59.10 | O |
| ATOM | 829 | N | THR | A | 118 | 65.313 | 43.524 | 78.746 | 1.00 | 58.87 | N |
| ATOM | 830 | CA | THR | A | 118 | 65.321 | 42.303 | 79.541 | 1.00 | 58.65 | C |
| ATOM | 831 | CB | THR | A | 118 | 66.450 | 42.343 | 80.606 | 1.00 | 58.45 | C |
| ATOM | 832 | OG1 | THR | A | 118 | 67.723 | 42.361 | 79.950 | 1.00 | 58.92 | O |
| ATOM | 833 | CG2 | THR | A | 118 | 66.378 | 41.130 | 81.521 | 1.00 | 57.28 | C |
| ATOM | 834 | C | THR | A | 118 | 63.971 | 42.060 | 80.225 | 1.00 | 59.06 | C |
| ATOM | 835 | O | THR | A | 118 | 63.530 | 40.915 | 80.346 | 1.00 | 58.98 | O |
| ATOM | 836 | N | ILE | A | 119 | 63.320 | 43.135 | 80.666 | 1.00 | 58.59 | N |
| ATOM | 837 | CA | ILE | A | 119 | 62.020 | 43.032 | 81.324 | 1.00 | 58.02 | C |
| ATOM | 838 | CB | ILE | A | 119 | 61.660 | 44.350 | 82.030 | 1.00 | 57.47 | C |
| ATOM | 839 | CG2 | ILE | A | 119 | 60.188 | 44.359 | 82.424 | 1.00 | 56.72 | C |
| ATOM | 840 | CG1 | ILE | A | 119 | 62.556 | 44.532 | 83.250 | 1.00 | 56.32 | C |
| ATOM | 841 | CD1 | ILE | A | 119 | 62.393 | 45.856 | 83.928 | 1.00 | 55.30 | C |
| ATOM | 842 | C | ILE | A | 119 | 60.926 | 42.697 | 80.309 | 1.00 | 58.48 | C |
| ATOM | 843 | O | ILE | A | 119 | 60.034 | 41.889 | 80.573 | 1.00 | 58.40 | O |
| ATOM | 844 | N | ARG | A | 120 | 61.010 | 43.320 | 79.142 | 1.00 | 59.02 | N |
| ATOM | 845 | CA | ARG | A | 120 | 60.036 | 43.100 | 78.088 | 1.00 | 60.10 | C |
| ATOM | 846 | CB | ARG | A | 120 | 60.390 | 43.957 | 76.875 | 1.00 | 59.93 | C |
| ATOM | 847 | CG | ARG | A | 120 | 59.435 | 43.825 | 75.711 | 1.00 | 60.37 | C |
| ATOM | 848 | CD | ARG | A | 120 | 59.885 | 44.702 | 74.557 | 1.00 | 61.88 | C |
| ATOM | 849 | NE | ARG | A | 120 | 61.121 | 44.236 | 73.934 | 1.00 | 63.57 | N |
| ATOM | 850 | CZ | ARG | A | 120 | 61.927 | 45.008 | 73.207 | 1.00 | 63.99 | C |
| ATOM | 851 | NH1 | ARG | A | 120 | 61.626 | 46.284 | 73.020 | 1.00 | 63.83 | N |
| ATOM | 852 | NH2 | ARG | A | 120 | 63.030 | 44.506 | 72.660 | 1.00 | 64.14 | N |
| ATOM | 853 | C | ARG | A | 120 | 60.006 | 41.631 | 77.692 | 1.00 | 60.26 | C |
| ATOM | 854 | O | ARG | A | 120 | 58.943 | 41.019 | 77.591 | 1.00 | 59.74 | O |
| ATOM | 855 | N | ASN | A | 121 | 61.189 | 41.072 | 77.473 | 1.00 | 61.17 | N |
| ATOM | 856 | CA | ASN | A | 121 | 61.307 | 39.679 | 77.083 | 1.00 | 61.50 | C |
| ATOM | 857 | CB | ASN | A | 121 | 62.718 | 39.393 | 76.577 | 1.00 | 62.76 | C |
| ATOM | 858 | CG | ASN | A | 121 | 63.041 | 40.170 | 75.309 | 1.00 | 64.18 | C |
| ATOM | 859 | OD1 | ASN | A | 121 | 62.142 | 40.673 | 74.626 | 1.00 | 64.22 | O |
| ATOM | 860 | ND2 | ASN | A | 121 | 64.322 | 40.260 | 74.980 | 1.00 | 65.15 | N |
| ATOM | 861 | C | ASN | A | 121 | 60.938 | 38.738 | 78.218 | 1.00 | 60.78 | C |
| ATOM | 862 | O | ASN | A | 121 | 60.480 | 37.625 | 77.979 | 1.00 | 61.19 | O |
| ATOM | 863 | N | ARG | A | 122 | 61.114 | 39.191 | 79.451 | 1.00 | 60.01 | N |
| ATOM | 864 | CA | ARG | A | 122 | 60.764 | 38.372 | 80.602 | 1.00 | 59.70 | C |
| ATOM | 865 | CB | ARG | A | 122 | 61.227 | 39.055 | 81.893 | 1.00 | 59.67 | C |
| ATOM | 866 | CG | ARG | A | 122 | 60.809 | 38.350 | 83.180 | 1.00 | 59.88 | C |
| ATOM | 867 | CD | ARG | A | 122 | 61.272 | 39.146 | 84.387 | 1.00 | 61.18 | C |
| ATOM | 868 | NE | ARG | A | 122 | 62.725 | 39.277 | 84.394 | 1.00 | 62.51 | N |
| ATOM | 869 | CZ | ARG | A | 122 | 63.379 | 40.366 | 84.778 | 1.00 | 63.48 | C |
| ATOM | 870 | NH1 | ARG | A | 122 | 62.709 | 41.436 | 85.191 | 1.00 | 63.85 | N |
| ATOM | 871 | NH2 | ARG | A | 122 | 64.704 | 40.383 | 84.738 | 1.00 | 62.95 | N |
| ATOM | 872 | C | ARG | A | 122 | 59.253 | 38.118 | 80.666 | 1.00 | 59.05 | C |
| ATOM | 873 | O | ARG | A | 122 | 58.811 | 37.111 | 81.218 | 1.00 | 59.35 | O |
| ATOM | 874 | N | HIS | A | 123 | 58.466 | 39.028 | 80.095 | 1.00 | 58.34 | N |
| ATOM | 875 | CA | HIS | A | 123 | 57.005 | 38.905 | 80.103 | 1.00 | 57.16 | C |
| ATOM | 876 | CB | HIS | A | 123 | 56.368 | 40.239 | 80.499 | 1.00 | 55.79 | C |
| ATOM | 877 | CG | HIS | A | 123 | 56.742 | 40.712 | 81.867 | 1.00 | 54.11 | C |
| ATOM | 878 | CD2 | HIS | A | 123 | 57.925 | 41.137 | 82.371 | 1.00 | 53.76 | C |
| ATOM | 879 | ND1 | HIS | A | 123 | 55.839 | 40.775 | 82.904 | 1.00 | 53.77 | N |
| ATOM | 880 | CE1 | HIS | A | 123 | 56.449 | 41.217 | 83.989 | 1.00 | 52.85 | C |
| ATOM | 881 | NE2 | HIS | A | 123 | 57.715 | 41.444 | 83.693 | 1.00 | 53.50 | N |
| ATOM | 882 | C | HIS | A | 123 | 56.417 | 38.482 | 78.760 | 1.00 | 57.39 | C |
| ATOM | 883 | O | HIS | A | 123 | 55.201 | 38.439 | 78.601 | 1.00 | 57.05 | O |
| ATOM | 884 | N | ASN | A | 124 | 57.276 | 38.171 | 77.799 | 1.00 | 58.14 | N |
| ATOM | 885 | CA | ASN | A | 124 | 56.810 | 37.800 | 76.472 | 1.00 | 59.74 | C |
| ATOM | 886 | CB | ASN | A | 124 | 57.992 | 37.427 | 75.581 | 1.00 | 61.01 | C |
| ATOM | 887 | CG | ASN | A | 124 | 58.366 | 38.539 | 74.630 | 1.00 | 63.01 | C |
| ATOM | 888 | OD1 | ASN | A | 124 | 57.513 | 39.071 | 73.919 | 1.00 | 64.73 | O |
| ATOM | 889 | ND2 | ASN | A | 124 | 59.645 | 38.898 | 74.604 | 1.00 | 64.47 | N |
| ATOM | 890 | C | ASN | A | 124 | 55.757 | 36.712 | 76.360 | 1.00 | 59.67 | C |
| ATOM | 891 | O | ASN | A | 124 | 54.956 | 36.736 | 75.432 | 1.00 | 60.01 | O |
| ATOM | 892 | N | ASP | A | 125 | 55.733 | 35.761 | 77.285 | 1.00 | 60.02 | N |
| ATOM | 893 | CA | ASP | A | 125 | 54.753 | 34.688 | 77.179 | 1.00 | 60.01 | C |
| ATOM | 894 | CB | ASP | A | 125 | 55.449 | 33.323 | 77.263 | 1.00 | 61.07 | C |
| ATOM | 895 | CG | ASP | A | 125 | 56.274 | 33.012 | 76.027 | 1.00 | 61.90 | C |
| ATOM | 896 | OD1 | ASP | A | 125 | 55.763 | 33.202 | 74.900 | 1.00 | 62.54 | O |
| ATOM | 897 | OD2 | ASP | A | 125 | 57.432 | 32.572 | 76.179 | 1.00 | 62.24 | O |
| ATOM | 898 | C | ASP | A | 125 | 53.621 | 34.734 | 78.187 | 1.00 | 59.81 | C |
| ATOM | 899 | O | ASP | A | 125 | 53.126 | 33.685 | 78.605 | 1.00 | 59.44 | O |
| ATOM | 900 | N | VAL | A | 126 | 53.197 | 35.938 | 78.562 | 1.00 | 59.08 | N |
| ATOM | 901 | CA | VAL | A | 126 | 52.115 | 36.087 | 79.528 | 1.00 | 58.68 | C |
| ATOM | 902 | CB | VAL | A | 126 | 51.907 | 37.567 | 79.945 | 1.00 | 59.76 | C |
| ATOM | 903 | CG1 | VAL | A | 126 | 50.652 | 37.699 | 80.804 | 1.00 | 59.96 | C |
| ATOM | 904 | CG2 | VAL | A | 126 | 53.098 | 38.053 | 80.728 | 1.00 | 60.17 | C |
| ATOM | 905 | C | VAL | A | 126 | 50.775 | 35.565 | 79.036 | 1.00 | 57.68 | C |
| ATOM | 906 | O | VAL | A | 126 | 50.084 | 34.837 | 79.747 | 1.00 | 57.74 | O |
| ATOM | 907 | N | VAL | A | 127 | 50.398 | 35.949 | 77.824 | 1.00 | 56.87 | N |
| ATOM | 908 | CA | VAL | A | 127 | 49.109 | 35.538 | 77.288 | 1.00 | 56.05 | C |
| ATOM | 909 | CB | VAL | A | 127 | 48.929 | 36.019 | 75.828 | 1.00 | 55.86 | C |
| ATOM | 910 | CG1 | VAL | A | 127 | 47.580 | 35.573 | 75.296 | 1.00 | 55.75 | C |
| ATOM | 911 | CG2 | VAL | A | 127 | 49.024 | 37.543 | 75.774 | 1.00 | 55.76 | C |
| ATOM | 912 | C | VAL | A | 127 | 48.851 | 34.033 | 77.386 | 1.00 | 55.37 | C |
| ATOM | 913 | O | VAL | A | 127 | 47.815 | 33.617 | 77.895 | 1.00 | 55.95 | O |
| ATOM | 914 | N | PRO | A | 128 | 49.783 | 33.197 | 76.908 | 1.00 | 54.49 | N |
| ATOM | 915 | CD | PRO | A | 128 | 51.029 | 33.509 | 76.194 | 1.00 | 54.62 | C |
| ATOM | 916 | CA | PRO | A | 128 | 49.571 | 31.747 | 76.987 | 1.00 | 53.59 | C |
| ATOM | 917 | CB | PRO | A | 128 | 50.743 | 31.183 | 76.192 | 1.00 | 54.14 | C |
| ATOM | 918 | CG | PRO | A | 128 | 51.152 | 32.324 | 75.293 | 1.00 | 54.37 | C |
| ATOM | 919 | C | PRO | A | 128 | 49.575 | 31.255 | 78.440 | 1.00 | 53.55 | C |
| ATOM | 920 | O | PRO | A | 128 | 48.865 | 30.307 | 78.791 | 1.00 | 53.05 | O |
| ATOM | 921 | N | THR | A | 129 | 50.393 | 31.902 | 79.269 | 1.00 | 53.19 | N |
| ATOM | 922 | CA | THR | A | 129 | 50.509 | 31.565 | 80.682 | 1.00 | 53.19 | C |
| ATOM | 923 | CB | THR | A | 129 | 51.604 | 32.404 | 81.361 | 1.00 | 53.34 | C |
| ATOM | 924 | OG1 | THR | A | 129 | 52.864 | 32.148 | 80.731 | 1.00 | 54.02 | O |
| ATOM | 925 | CG2 | THR | A | 129 | 51.703 | 32.070 | 82.835 | 1.00 | 52.83 | C |
| ATOM | 926 | C | THR | A | 129 | 49.177 | 31.832 | 81.375 | 1.00 | 53.82 | C |
| ATOM | 927 | O | THR | A | 129 | 48.644 | 30.965 | 82.067 | 1.00 | 53.80 | O |
| ATOM | 928 | N | MET | A | 130 | 48.638 | 33.034 | 81.182 | 1.00 | 54.08 | N |
| ATOM | 929 | CA | MET | A | 130 | 47.358 | 33.399 | 81.783 | 1.00 | 54.75 | C |
| ATOM | 930 | CB | MET | A | 130 | 47.050 | 34.881 | 81.522 | 1.00 | 54.90 | C |
| ATOM | 931 | CG | MET | A | 130 | 47.888 | 35.833 | 82.351 | 1.00 | 55.57 | C |
| ATOM | 932 | SD | MET | A | 130 | 47.413 | 35.775 | 84.080 | 1.00 | 56.31 | S |
| ATOM | 933 | CE | MET | A | 130 | 48.055 | 37.302 | 84.660 | 1.00 | 56.95 | C |
| ATOM | 934 | C | MET | A | 130 | 46.225 | 32.526 | 81.243 | 1.00 | 54.85 | C |
| ATOM | 935 | O | MET | A | 130 | 45.311 | 32.147 | 81.978 | 1.00 | 54.72 | O |
| ATOM | 936 | N | ALA | A | 131 | 46.285 | 32.212 | 79.956 | 1.00 | 54.60 | N |
| ATOM | 937 | CA | ALA | A | 131 | 45.269 | 31.374 | 79.321 | 1.00 | 54.78 | C |
| ATOM | 938 | CB | ALA | A | 131 | 45.617 | 31.181 | 77.863 | 1.00 | 54.66 | C |
| ATOM | 939 | C | ALA | A | 131 | 45.207 | 30.012 | 80.011 | 1.00 | 54.62 | C |
| ATOM | 940 | O | ALA | A | 131 | 44.155 | 29.366 | 80.065 | 1.00 | 53.86 | O |
| ATOM | 941 | N | GLN | A | 132 | 46.352 | 29.578 | 80.526 | 1.00 | 54.84 | N |
| ATOM | 942 | CA | GLN | A | 132 | 46.445 | 28.299 | 81.212 | 1.00 | 54.08 | C |
| ATOM | 943 | CB | GLN | A | 132 | 47.911 | 27.907 | 81.372 | 1.00 | 54.69 | C |
| ATOM | 944 | CG | GLN | A | 132 | 48.138 | 26.413 | 81.557 | 1.00 | 57.87 | C |
| ATOM | 945 | CD | GLN | A | 132 | 47.755 | 25.596 | 80.330 | 1.00 | 58.14 | C |
| ATOM | 946 | OE1 | GLN | A | 132 | 48.159 | 25.910 | 79.208 | 1.00 | 59.48 | O |
| ATOM | 947 | NE2 | GLN | A | 132 | 46.986 | 24.536 | 80.541 | 1.00 | 58.57 | N |
| ATOM | 948 | C | GLN | A | 132 | 45.759 | 28.368 | 82.584 | 1.00 | 53.45 | C |
| ATOM | 949 | O | GLN | A | 132 | 45.258 | 27.359 | 83.093 | 1.00 | 52.47 | O |
| ATOM | 950 | N | GLY | A | 133 | 45.731 | 29.560 | 83.175 | 1.00 | 51.79 | N |
| ATOM | 951 | CA | GLY | A | 133 | 45.083 | 29.724 | 84.460 | 1.00 | 50.92 | C |
| ATOM | 952 | C | GLY | A | 133 | 43.584 | 29.766 | 84.243 | 1.00 | 50.92 | C |
| ATOM | 953 | O | GLY | A | 133 | 42.800 | 29.335 | 85.089 | 1.00 | 51.41 | O |
| ATOM | 954 | N | VAL | A | 134 | 43.178 | 30.297 | 83.096 | 1.00 | 51.63 | N |
| ATOM | 955 | CA | VAL | A | 134 | 41.761 | 30.375 | 82.761 | 1.00 | 51.83 | C |
| ATOM | 956 | CB | VAL | A | 134 | 41.540 | 31.106 | 81.427 | 1.00 | 51.15 | C |
| ATOM | 957 | CG1 | VAL | A | 134 | 40.074 | 31.013 | 81.022 | 1.00 | 51.26 | C |
| ATOM | 958 | CG2 | VAL | A | 134 | 41.976 | 32.559 | 81.558 | 1.00 | 51.03 | C |
| ATOM | 959 | C | VAL | A | 134 | 41.240 | 28.953 | 82.635 | 1.00 | 51.93 | C |
| ATOM | 960 | O | VAL | A | 134 | 40.187 | 28.608 | 83.166 | 1.00 | 51.99 | O |
| ATOM | 961 | N | LEU | A | 135 | 42.000 | 28.135 | 81.923 | 1.00 | 52.52 | N |
| ATOM | 962 | CA | LEU | A | 135 | 41.648 | 26.743 | 81.716 | 1.00 | 52.95 | C |
| ATOM | 963 | CB | LEU | A | 135 | 42.680 | 26.094 | 80.799 | 1.00 | 53.59 | C |
| ATOM | 964 | CG | LEU | A | 135 | 42.160 | 24.951 | 79.928 | 1.00 | 54.46 | C |
| ATOM | 965 | CD1 | LEU | A | 135 | 40.712 | 25.200 | 79.503 | 1.00 | 53.75 | C |
| ATOM | 966 | CD2 | LEU | A | 135 | 43.063 | 24.841 | 78.723 | 1.00 | 55.09 | C |
| ATOM | 967 | C | LEU | A | 135 | 41.596 | 26.016 | 83.059 | 1.00 | 52.69 | C |
| ATOM | 968 | O | LEU | A | 135 | 40.646 | 25.289 | 83.350 | 1.00 | 51.74 | O |
| ATOM | 969 | N | GLU | A | 136 | 42.616 | 26.222 | 83.882 | 1.00 | 52.77 | N |
| ATOM | 970 | CA | GLU | A | 136 | 42.636 | 25.584 | 85.186 | 1.00 | 53.25 | C |
| ATOM | 971 | CB | GLU | A | 136 | 43.844 | 26.033 | 86.012 | 1.00 | 53.58 | C |
| ATOM | 972 | CG | GLU | A | 136 | 43.851 | 25.420 | 87.402 | 1.00 | 55.67 | C |
| ATOM | 973 | CD | GLU | A | 136 | 45.057 | 25.811 | 88.210 | 1.00 | 57.14 | C |
| ATOM | 974 | OE1 | GLU | A | 136 | 45.983 | 26.381 | 87.621 | 1.00 | 58.17 | O |
| ATOM | 975 | OE2 | GLU | A | 136 | 45.084 | 25.545 | 89.430 | 1.00 | 58.43 | O |
| ATOM | 976 | C | GLU | A | 136 | 41.355 | 25.947 | 85.925 | 1.00 | 53.73 | C |
| ATOM | 977 | O | GLU | A | 136 | 40.696 | 25.076 | 86.501 | 1.00 | 53.01 | O |
| ATOM | 978 | N | TYR | A | 137 | 40.999 | 27.231 | 85.890 | 1.00 | 53.71 | N |
| ATOM | 979 | CA | TYR | A | 137 | 39.799 | 27.707 | 86.561 | 1.00 | 53.72 | C |
| ATOM | 980 | CB | TYR | A | 137 | 39.692 | 29.235 | 86.453 | 1.00 | 53.54 | C |
| ATOM | 981 | CG | TYR | A | 137 | 38.578 | 29.806 | 87.298 | 1.00 | 52.05 | C |
| ATOM | 982 | CD1 | TYR | A | 137 | 37.247 | 29.612 | 86.948 | 1.00 | 51.85 | C |
| ATOM | 983 | CE1 | TYR | A | 137 | 36.213 | 30.028 | 87.777 | 1.00 | 51.68 | C |
| ATOM | 984 | CD2 | TYR | A | 137 | 38.852 | 30.449 | 88.500 | 1.00 | 52.00 | C |
| ATOM | 985 | CE2 | TYR | A | 137 | 37.825 | 30.873 | 89.338 | 1.00 | 52.25 | C |
| ATOM | 986 | CZ | TYR | A | 137 | 36.507 | 30.653 | 88.972 | 1.00 | 52.07 | C |
| ATOM | 987 | OH | TYR | A | 137 | 35.486 | 31.017 | 89.819 | 1.00 | 52.82 | O |
| ATOM | 988 | C | TYR | A | 137 | 38.534 | 27.054 | 86.002 | 1.00 | 54.84 | C |
| ATOM | 989 | O | TYR | A | 137 | 37.656 | 26.648 | 86.761 | 1.00 | 54.28 | O |
| ATOM | 990 | N | LYS | A | 138 | 38.445 | 26.966 | 84.678 | 1.00 | 56.56 | N |
| ATOM | 991 | CA | LYS | A | 138 | 37.300 | 26.357 | 84.005 | 1.00 | 58.63 | C |
| ATOM | 992 | CB | LYS | A | 138 | 37.531 | 26.314 | 82.489 | 1.00 | 59.06 | C |
| ATOM | 993 | CG | LYS | A | 138 | 36.950 | 27.479 | 81.706 | 1.00 | 60.57 | C |
| ATOM | 994 | CD | LYS | A | 138 | 36.053 | 26.964 | 80.582 | 1.00 | 61.30 | C |
| ATOM | 995 | CE | LYS | A | 138 | 36.675 | 27.137 | 79.206 | 1.00 | 61.35 | C |
| ATOM | 996 | NZ | LYS | A | 138 | 35.823 | 26.505 | 78.156 | 1.00 | 61.44 | N |
| ATOM | 997 | C | LYS | A | 138 | 37.041 | 24.926 | 84.487 | 1.00 | 60.21 | C |
| ATOM | 998 | O | LYS | A | 138 | 35.897 | 24.513 | 84.685 | 1.00 | 60.11 | O |
| ATOM | 999 | N | ASP | A | 139 | 38.115 | 24.170 | 84.666 | 1.00 | 61.53 | N |
| ATOM | 1000 | CA | ASP | A | 139 | 37.997 | 22.788 | 85.093 | 1.00 | 63.03 | C |
| ATOM | 1001 | CB | ASP | A | 139 | 39.087 | 21.959 | 84.410 | 1.00 | 63.16 | C |
| ATOM | 1002 | CG | ASP | A | 139 | 38.926 | 21.940 | 82.899 | 1.00 | 63.89 | C |
| ATOM | 1003 | OD1 | ASP | A | 139 | 37.764 | 21.926 | 82.436 | 1.00 | 63.94 | O |
| ATOM | 1004 | OD2 | ASP | A | 139 | 39.943 | 21.932 | 82.176 | 1.00 | 64.57 | O |
| ATOM | 1005 | C | ASP | A | 139 | 38.025 | 22.600 | 86.606 | 1.00 | 63.61 | C |
| ATOM | 1006 | O | ASP | A | 139 | 37.518 | 21.609 | 87.129 | 1.00 | 63.78 | O |
| ATOM | 1007 | N | THR | A | 140 | 38.594 | 23.566 | 87.312 | 1.00 | 64.47 | N |
| ATOM | 1008 | CA | THR | A | 140 | 38.667 | 23.486 | 88.756 | 1.00 | 65.77 | C |
| ATOM | 1009 | CB | THR | A | 140 | 39.907 | 24.234 | 89.271 | 1.00 | 65.96 | C |
| ATOM | 1010 | OG1 | THR | A | 140 | 41.087 | 23.546 | 88.836 | 1.00 | 67.64 | O |
| ATOM | 1011 | CG2 | THR | A | 140 | 39.909 | 24.289 | 90.780 | 1.00 | 67.49 | C |
| ATOM | 1012 | C | THR | A | 140 | 37.413 | 24.017 | 89.460 | 1.00 | 66.20 | C |
| ATOM | 1013 | O | THR | A | 140 | 37.004 | 23.468 | 90.478 | 1.00 | 65.71 | O |
| ATOM | 1014 | N | TYR | A | 141 | 36.799 | 25.071 | 88.921 | 1.00 | 67.28 | N |
| ATOM | 1015 | CA | TYR | A | 141 | 35.603 | 25.649 | 89.545 | 1.00 | 67.99 | C |
| ATOM | 1016 | CB | TYR | A | 141 | 35.934 | 27.013 | 90.166 | 1.00 | 67.62 | C |
| ATOM | 1017 | CG | TYR | A | 141 | 36.934 | 26.904 | 91.288 | 1.00 | 68.60 | C |
| ATOM | 1018 | CD1 | TYR | A | 141 | 38.271 | 27.254 | 91.097 | 1.00 | 68.59 | C |
| ATOM | 1019 | CE1 | TYR | A | 141 | 39.224 | 27.041 | 92.098 | 1.00 | 69.53 | C |
| ATOM | 1020 | CD2 | TYR | A | 141 | 36.566 | 26.346 | 92.514 | 1.00 | 68.95 | C |
| ATOM | 1021 | CE2 | TYR | A | 141 | 37.508 | 26.125 | 93.520 | 1.00 | 69.65 | C |
| ATOM | 1022 | CZ | TYR | A | 141 | 38.834 | 26.470 | 93.305 | 1.00 | 69.74 | C |
| ATOM | 1023 | OH | TYR | A | 141 | 39.771 | 26.210 | 94.278 | 1.00 | 70.68 | O |
| ATOM | 1024 | C | TYR | A | 141 | 34.386 | 25.787 | 88.643 | 1.00 | 68.53 | C |
| ATOM | 1025 | O | TYR | A | 141 | 33.287 | 26.065 | 89.120 | 1.00 | 68.23 | O |
| ATOM | 1026 | N | GLY | A | 142 | 34.570 | 25.592 | 87.344 | 1.00 | 69.55 | N |
| ATOM | 1027 | CA | GLY | A | 142 | 33.444 | 25.702 | 86.434 | 1.00 | 71.09 | C |
| ATOM | 1028 | C | GLY | A | 142 | 33.394 | 27.017 | 85.683 | 1.00 | 72.27 | C |
| ATOM | 1029 | O | GLY | A | 142 | 34.123 | 27.956 | 86.004 | 1.00 | 72.09 | O |
| ATOM | 1030 | N | ASP | A | 143 | 32.522 | 27.084 | 84.684 | 1.00 | 73.53 | N |
| ATOM | 1031 | CA | ASP | A | 143 | 32.383 | 28.281 | 83.864 | 1.00 | 75.10 | C |
| ATOM | 1032 | CB | ASP | A | 143 | 32.742 | 27.954 | 82.408 | 1.00 | 76.30 | C |
| ATOM | 1033 | CG | ASP | A | 143 | 31.841 | 26.889 | 81.805 | 1.00 | 78.14 | C |
| ATOM | 1034 | OD1 | ASP | A | 143 | 31.344 | 26.021 | 82.560 | 1.00 | 78.42 | O |
| ATOM | 1035 | OD2 | ASP | A | 143 | 31.643 | 26.912 | 80.570 | 1.00 | 79.88 | O |
| ATOM | 1036 | C | ASP | A | 143 | 30.994 | 28.906 | 83.942 | 1.00 | 75.47 | C |
| ATOM | 1037 | O | ASP | A | 143 | 30.168 | 28.740 | 83.047 | 1.00 | 75.87 | O |
| ATOM | 1038 | N | ASP | A | 144 | 30.750 | 29.635 | 85.024 | 1.00 | 75.67 | N |
| ATOM | 1039 | CA | ASP | A | 144 | 29.474 | 30.306 | 85.236 | 1.00 | 76.56 | C |
| ATOM | 1040 | CB | ASP | A | 144 | 29.011 | 30.083 | 86.679 | 1.00 | 76.82 | C |
| ATOM | 1041 | CG | ASP | A | 144 | 30.100 | 30.378 | 87.683 | 1.00 | 77.63 | C |
| ATOM | 1042 | OD1 | ASP | A | 144 | 31.126 | 29.662 | 87.672 | 1.00 | 78.87 | O |
| ATOM | 1043 | OD2 | ASP | A | 144 | 29.937 | 31.330 | 88.476 | 1.00 | 77.82 | O |
| ATOM | 1044 | C | ASP | A | 144 | 29.597 | 31.810 | 84.944 | 1.00 | 76.17 | C |
| ATOM | 1045 | O | ASP | A | 144 | 30.680 | 32.392 | 85.051 | 1.00 | 76.70 | O |
| ATOM | 1046 | N | PRO | A | 145 | 28.482 | 32.455 | 84.569 | 1.00 | 75.55 | N |
| ATOM | 1047 | CD | PRO | A | 145 | 27.167 | 31.834 | 84.329 | 1.00 | 75.47 | C |
| ATOM | 1048 | CA | PRO | A | 145 | 28.434 | 33.883 | 84.256 | 1.00 | 74.77 | C |
| ATOM | 1049 | CB | PRO | A | 145 | 26.943 | 34.172 | 84.242 | 1.00 | 74.69 | C |
| ATOM | 1050 | CG | PRO | A | 145 | 26.412 | 32.941 | 83.600 | 1.00 | 75.31 | C |
| ATOM | 1051 | C | PRO | A | 145 | 29.198 | 34.803 | 85.202 | 1.00 | 73.96 | C |
| ATOM | 1052 | O | PRO | A | 145 | 29.904 | 35.707 | 84.750 | 1.00 | 74.21 | O |
| ATOM | 1053 | N | VAL | A | 146 | 29.066 | 34.589 | 86.506 | 1.00 | 72.86 | N |
| ATOM | 1054 | CA | VAL | A | 146 | 29.777 | 35.450 | 87.432 | 1.00 | 71.74 | C |
| ATOM | 1055 | CB | VAL | A | 146 | 29.334 | 35.213 | 88.884 | 1.00 | 71.59 | C |
| ATOM | 1056 | CG1 | VAL | A | 146 | 30.152 | 36.087 | 89.837 | 1.00 | 71.13 | C |
| ATOM | 1057 | CG2 | VAL | A | 146 | 27.861 | 35.546 | 89.016 | 1.00 | 71.10 | C |
| ATOM | 1058 | C | VAL | A | 146 | 31.288 | 35.279 | 87.312 | 1.00 | 71.10 | C |
| ATOM | 1059 | O | VAL | A | 146 | 32.006 | 36.259 | 87.101 | 1.00 | 70.79 | O |
| ATOM | 1060 | N | SER | A | 147 | 31.779 | 34.049 | 87.427 | 1.00 | 70.12 | N |
| ATOM | 1061 | CA | SER | A | 147 | 33.218 | 33.846 | 87.321 | 1.00 | 69.71 | C |
| ATOM | 1062 | CB | SER | A | 147 | 33.597 | 32.371 | 87.566 | 1.00 | 69.24 | C |
| ATOM | 1063 | OG | SER | A | 147 | 33.009 | 31.497 | 86.626 | 1.00 | 68.44 | O |
| ATOM | 1064 | C | SER | A | 147 | 33.728 | 34.337 | 85.961 | 1.00 | 69.18 | C |
| ATOM | 1065 | O | SER | A | 147 | 34.867 | 34.787 | 85.853 | 1.00 | 68.89 | O |
| ATOM | 1066 | N | ASN | A | 148 | 32.890 | 34.270 | 84.928 | 1.00 | 68.35 | N |
| ATOM | 1067 | CA | ASN | A | 148 | 33.303 | 34.755 | 83.611 | 1.00 | 68.05 | C |
| ATOM | 1068 | CB | ASN | A | 148 | 32.287 | 34.404 | 82.525 | 1.00 | 68.31 | C |
| ATOM | 1069 | CG | ASN | A | 148 | 32.602 | 35.092 | 81.197 | 1.00 | 68.92 | C |
| ATOM | 1070 | OD1 | ASN | A | 148 | 33.496 | 34.673 | 80.460 | 1.00 | 68.88 | O |
| ATOM | 1071 | ND2 | ASN | A | 148 | 31.880 | 36.168 | 80.902 | 1.00 | 68.71 | N |
| ATOM | 1072 | C | ASN | A | 148 | 33.441 | 36.272 | 83.626 | 1.00 | 67.09 | C |
| ATOM | 1073 | O | ASN | A | 148 | 34.392 | 36.822 | 83.076 | 1.00 | 66.96 | O |
| ATOM | 1074 | N | GLN | A | 149 | 32.471 | 36.940 | 84.240 | 1.00 | 66.56 | N |
| ATOM | 1075 | CA | GLN | A | 149 | 32.469 | 38.392 | 84.318 | 1.00 | 65.91 | C |
| ATOM | 1076 | CB | GLN | A | 149 | 31.199 | 38.875 | 85.016 | 1.00 | 67.91 | C |
| ATOM | 1077 | CG | GLN | A | 149 | 30.591 | 40.112 | 84.389 | 1.00 | 70.80 | C |
| ATOM | 1078 | CD | GLN | A | 149 | 30.252 | 39.886 | 82.929 | 1.00 | 72.94 | C |
| ATOM | 1079 | OE1 | GLN | A | 149 | 30.917 | 40.416 | 82.029 | 1.00 | 74.27 | O |
| ATOM | 1080 | NE2 | GLN | A | 149 | 29.221 | 39.082 | 82.682 | 1.00 | 73.67 | N |
| ATOM | 1081 | C | GLN | A | 149 | 33.686 | 38.884 | 85.086 | 1.00 | 64.41 | C |
| ATOM | 1082 | O | GLN | A | 149 | 34.343 | 39.847 | 84.686 | 1.00 | 64.39 | O |
| ATOM | 1083 | N | ASN | A | 150 | 33.979 | 38.220 | 86.197 | 1.00 | 62.37 | N |
| ATOM | 1084 | CA | ASN | A | 150 | 35.115 | 38.591 | 87.025 | 1.00 | 60.49 | C |
| ATOM | 1085 | CB | ASN | A | 150 | 35.098 | 37.778 | 88.315 | 1.00 | 61.87 | C |
| ATOM | 1086 | CG | ASN | A | 150 | 33.939 | 38.154 | 89.206 | 1.00 | 63.75 | C |
| ATOM | 1087 | OD1 | ASN | A | 150 | 33.831 | 39.301 | 89.639 | 1.00 | 64.84 | O |
| ATOM | 1088 | ND2 | ASN | A | 150 | 33.053 | 37.196 | 89.477 | 1.00 | 64.65 | N |
| ATOM | 1089 | C | ASN | A | 150 | 36.427 | 38.399 | 86.281 | 1.00 | 58.15 | C |
| ATOM | 1090 | O | ASN | A | 150 | 37.299 | 39.263 | 86.324 | 1.00 | 57.30 | O |
| ATOM | 1091 | N | ILE | A | 151 | 36.556 | 37.263 | 85.603 | 1.00 | 55.52 | N |
| ATOM | 1092 | CA | ILE | A | 151 | 37.748 | 36.948 | 84.820 | 1.00 | 53.49 | C |
| ATOM | 1093 | CB | ILE | A | 151 | 37.668 | 35.499 | 84.266 | 1.00 | 52.68 | C |
| ATOM | 1094 | CG2 | ILE | A | 151 | 38.719 | 35.284 | 83.178 | 1.00 | 52.20 | C |
| ATOM | 1095 | CG1 | ILE | A | 151 | 37.845 | 34.498 | 85.414 | 1.00 | 53.35 | C |
| ATOM | 1096 | CD1 | ILE | A | 151 | 37.577 | 33.031 | 85.040 | 1.00 | 52.31 | C |
| ATOM | 1097 | C | ILE | A | 151 | 37.884 | 37.940 | 83.657 | 1.00 | 52.94 | C |
| ATOM | 1098 | O | ILE | A | 151 | 38.983 | 38.416 | 83.355 | 1.00 | 51.23 | O |
| ATOM | 1099 | N | GLN | A | 152 | 36.755 | 38.247 | 83.022 | 1.00 | 52.07 | N |
| ATOM | 1100 | CA | GLN | A | 152 | 36.705 | 39.182 | 81.904 | 1.00 | 52.21 | C |
| ATOM | 1101 | CB | GLN | A | 152 | 35.272 | 39.290 | 81.378 | 1.00 | 53.65 | C |
| ATOM | 1102 | CG | GLN | A | 152 | 35.146 | 39.887 | 79.991 | 1.00 | 54.79 | C |
| ATOM | 1103 | CD | GLN | A | 152 | 35.776 | 39.006 | 78.937 | 1.00 | 55.64 | C |
| ATOM | 1104 | OE1 | GLN | A | 152 | 35.785 | 37.786 | 79.067 | 1.00 | 55.56 | O |
| ATOM | 1105 | NE2 | GLN | A | 152 | 36.292 | 39.617 | 77.877 | 1.00 | 56.95 | N |
| ATOM | 1106 | C | GLN | A | 152 | 37.164 | 40.549 | 82.387 | 1.00 | 51.54 | C |
| ATOM | 1107 | O | GLN | A | 152 | 38.028 | 41.182 | 81.780 | 1.00 | 51.60 | O |
| ATOM | 1108 | N | TYR | A | 153 | 36.573 | 40.992 | 83.490 | 1.00 | 50.63 | N |
| ATOM | 1109 | CA | TYR | A | 153 | 36.905 | 42.278 | 84.079 | 1.00 | 49.85 | C |
| ATOM | 1110 | CB | TYR | A | 153 | 36.106 | 42.489 | 85.369 | 1.00 | 51.25 | C |
| ATOM | 1111 | CG | TYR | A | 153 | 36.374 | 43.804 | 86.061 | 1.00 | 53.21 | C |
| ATOM | 1112 | CD1 | TYR | A | 153 | 35.716 | 44.974 | 85.667 | 1.00 | 54.95 | C |
| ATOM | 1113 | CE1 | TYR | A | 153 | 35.966 | 46.195 | 86.304 | 1.00 | 55.65 | C |
| ATOM | 1114 | CD2 | TYR | A | 153 | 37.292 | 43.884 | 87.108 | 1.00 | 54.51 | C |
| ATOM | 1115 | CE2 | TYR | A | 153 | 37.553 | 45.098 | 87.752 | 1.00 | 55.93 | C |
| ATOM | 1116 | CZ | TYR | A | 153 | 36.888 | 46.246 | 87.345 | 1.00 | 56.55 | C |
| ATOM | 1117 | OH | TYR | A | 153 | 37.156 | 47.439 | 87.982 | 1.00 | 59.20 | O |
| ATOM | 1118 | C | TYR | A | 153 | 38.398 | 42.343 | 84.385 | 1.00 | 48.23 | C |
| ATOM | 1119 | O | TYR | A | 153 | 39.065 | 43.317 | 84.039 | 1.00 | 48.51 | O |
| ATOM | 1120 | N | PHE | A | 154 | 38.924 | 41.296 | 85.020 | 1.00 | 45.96 | N |
| ATOM | 1121 | CA | PHE | A | 154 | 40.334 | 41.265 | 85.385 | 1.00 | 43.25 | C |
| ATOM | 1122 | CB | PHE | A | 154 | 40.618 | 40.119 | 86.365 | 1.00 | 43.37 | C |
| ATOM | 1123 | CG | PHE | A | 154 | 42.078 | 39.888 | 86.594 | 1.00 | 42.80 | C |
| ATOM | 1124 | CD1 | PHE | A | 154 | 42.745 | 38.867 | 85.928 | 1.00 | 42.98 | C |
| ATOM | 1125 | CD2 | PHE | A | 154 | 42.814 | 40.769 | 87.376 | 1.00 | 42.91 | C |
| ATOM | 1126 | CE1 | PHE | A | 154 | 44.128 | 38.735 | 86.030 | 1.00 | 43.07 | C |
| ATOM | 1127 | CE2 | PHE | A | 154 | 44.195 | 40.647 | 87.485 | 1.00 | 42.81 | C |
| ATOM | 1128 | CZ | PHE | A | 154 | 44.855 | 39.630 | 86.808 | 1.00 | 42.95 | C |
| ATOM | 1129 | C | PHE | A | 154 | 41.308 | 41.178 | 84.209 | 1.00 | 41.88 | C |
| ATOM | 1130 | O | PHE | A | 154 | 42.265 | 41.941 | 84.138 | 1.00 | 40.70 | O |
| ATOM | 1131 | N | LEU | A | 155 | 41.077 | 40.245 | 83.296 | 1.00 | 40.78 | N |
| ATOM | 1132 | CA | LEU | A | 155 | 41.959 | 40.087 | 82.143 | 1.00 | 40.15 | C |
| ATOM | 1133 | CB | LEU | A | 155 | 41.606 | 38.809 | 81.373 | 1.00 | 40.59 | C |
| ATOM | 1134 | CG | LEU | A | 155 | 41.918 | 37.531 | 82.156 | 1.00 | 41.43 | C |
| ATOM | 1135 | CD1 | LEU | A | 155 | 41.531 | 36.311 | 81.346 | 1.00 | 40.07 | C |
| ATOM | 1136 | CD2 | LEU | A | 155 | 43.410 | 37.495 | 82.491 | 1.00 | 41.61 | C |
| ATOM | 1137 | C | LEU | A | 155 | 41.932 | 41.295 | 81.211 | 1.00 | 39.13 | C |
| ATOM | 1138 | O | LEU | A | 155 | 42.949 | 41.648 | 80.622 | 1.00 | 37.96 | O |
| ATOM | 1139 | N | ASP | A | 156 | 40.773 | 41.925 | 81.068 | 1.00 | 38.36 | N |
| ATOM | 1140 | CA | ASP | A | 156 | 40.702 | 43.113 | 80.228 | 1.00 | 38.37 | C |
| ATOM | 1141 | CB | ASP | A | 156 | 39.270 | 43.664 | 80.158 | 1.00 | 38.24 | C |
| ATOM | 1142 | CG | ASP | A | 156 | 38.461 | 43.065 | 79.018 | 1.00 | 38.13 | C |
| ATOM | 1143 | OD1 | ASP | A | 156 | 39.062 | 42.478 | 78.109 | 1.00 | 37.77 | O |
| ATOM | 1144 | OD2 | ASP | A | 156 | 37.224 | 43.202 | 79.022 | 1.00 | 42.71 | O |
| ATOM | 1145 | C | ASP | A | 156 | 41.625 | 44.176 | 80.817 | 1.00 | 37.61 | C |
| ATOM | 1146 | O | ASP | A | 156 | 42.429 | 44.767 | 80.104 | 1.00 | 37.17 | O |
| ATOM | 1147 | N | ARG | A | 157 | 41.524 | 44.407 | 82.124 | 1.00 | 37.69 | N |
| ATOM | 1148 | CA | ARG | A | 157 | 42.361 | 45.416 | 82.764 | 1.00 | 38.51 | C |
| ATOM | 1149 | CB | ARG | A | 157 | 41.866 | 45.698 | 84.188 | 1.00 | 40.05 | C |
| ATOM | 1150 | CG | ARG | A | 157 | 40.522 | 46.446 | 84.222 | 1.00 | 42.10 | C |
| ATOM | 1151 | CD | ARG | A | 157 | 39.797 | 46.285 | 85.558 | 1.00 | 45.26 | C |
| ATOM | 1152 | NE | ARG | A | 157 | 40.491 | 46.938 | 86.669 | 1.00 | 48.72 | N |
| ATOM | 1153 | CZ | ARG | A | 157 | 40.497 | 48.253 | 86.884 | 1.00 | 49.84 | C |
| ATOM | 1154 | NH1 | ARG | A | 157 | 39.839 | 49.065 | 86.063 | 1.00 | 49.97 | N |
| ATOM | 1155 | NH2 | ARG | A | 157 | 41.166 | 48.754 | 87.918 | 1.00 | 50.47 | N |
| ATOM | 1156 | C | ARG | A | 157 | 43.824 | 45.013 | 82.767 | 1.00 | 38.66 | C |
| ATOM | 1157 | O | ARG | A | 157 | 44.696 | 45.822 | 82.427 | 1.00 | 38.23 | O |
| ATOM | 1158 | N | PHE | A | 158 | 44.092 | 43.755 | 83.123 | 1.00 | 39.26 | N |
| ATOM | 1159 | CA | PHE | A | 158 | 45.455 | 43.245 | 83.167 | 1.00 | 38.27 | C |
| ATOM | 1160 | CB | PHE | A | 158 | 45.482 | 41.765 | 83.560 | 1.00 | 40.33 | C |
| ATOM | 1161 | CG | PHE | A | 158 | 46.868 | 41.176 | 83.561 | 1.00 | 41.26 | C |
| ATOM | 1162 | CD1 | PHE | A | 158 | 47.748 | 41.444 | 84.605 | 1.00 | 41.35 | C |
| ATOM | 1163 | CD2 | PHE | A | 158 | 47.317 | 40.425 | 82.483 | 1.00 | 41.92 | C |
| ATOM | 1164 | CE1 | PHE | A | 158 | 49.061 | 40.980 | 84.578 | 1.00 | 42.09 | C |
| ATOM | 1165 | CE2 | PHE | A | 158 | 48.636 | 39.953 | 82.443 | 1.00 | 43.88 | C |
| ATOM | 1166 | CZ | PHE | A | 158 | 49.511 | 40.236 | 83.499 | 1.00 | 42.71 | C |
| ATOM | 1167 | C | PHE | A | 158 | 46.144 | 43.399 | 81.824 | 1.00 | 37.83 | C |
| ATOM | 1168 | O | PHE | A | 158 | 47.274 | 43.891 | 81.750 | 1.00 | 37.89 | O |
| ATOM | 1169 | N | TYR | A | 159 | 45.473 | 42.968 | 80.757 | 1.00 | 37.88 | N |
| ATOM | 1170 | CA | TYR | A | 159 | 46.048 | 43.081 | 79.421 | 1.00 | 38.64 | C |
| ATOM | 1171 | CB | TYR | A | 159 | 45.233 | 42.263 | 78.422 | 1.00 | 41.16 | C |
| ATOM | 1172 | CG | TYR | A | 159 | 45.451 | 40.775 | 78.549 | 1.00 | 42.40 | C |
| ATOM | 1173 | CD1 | TYR | A | 159 | 46.741 | 40.253 | 78.637 | 1.00 | 43.16 | C |
| ATOM | 1174 | CE1 | TYR | A | 159 | 46.955 | 38.888 | 78.749 | 1.00 | 44.93 | C |
| ATOM | 1175 | CD2 | TYR | A | 159 | 44.372 | 39.883 | 78.573 | 1.00 | 42.77 | C |
| ATOM | 1176 | CE2 | TYR | A | 159 | 44.574 | 38.504 | 78.681 | 1.00 | 42.78 | C |
| ATOM | 1177 | CZ | TYR | A | 159 | 45.871 | 38.017 | 78.769 | 1.00 | 44.13 | C |
| ATOM | 1178 | OH | TYR | A | 159 | 46.108 | 36.667 | 78.859 | 1.00 | 45.24 | O |
| ATOM | 1179 | C | TYR | A | 159 | 46.183 | 44.529 | 78.935 | 1.00 | 38.80 | C |
| ATOM | 1180 | O | TYR | A | 159 | 47.175 | 44.874 | 78.286 | 1.00 | 38.95 | O |
| ATOM | 1181 | N | LEU | A | 160 | 45.203 | 45.377 | 79.241 | 1.00 | 38.49 | N |
| ATOM | 1182 | CA | LEU | A | 160 | 45.305 | 46.774 | 78.833 | 1.00 | 39.74 | C |
| ATOM | 1183 | CB | LEU | A | 160 | 44.079 | 47.573 | 79.274 | 1.00 | 39.74 | C |
| ATOM | 1184 | CG | LEU | A | 160 | 43.396 | 48.399 | 78.177 | 1.00 | 41.67 | C |
| ATOM | 1185 | CD1 | LEU | A | 160 | 42.488 | 49.435 | 78.836 | 1.00 | 42.05 | C |
| ATOM | 1186 | CD2 | LEU | A | 160 | 44.426 | 49.086 | 77.288 | 1.00 | 39.95 | C |
| ATOM | 1187 | C | LEU | A | 160 | 46.554 | 47.346 | 79.508 | 1.00 | 39.07 | C |
| ATOM | 1188 | O | LEU | A | 160 | 47.406 | 47.971 | 78.864 | 1.00 | 38.65 | O |
| ATOM | 1189 | N | SER | A | 161 | 46.661 | 47.106 | 80.811 | 1.00 | 39.49 | N |
| ATOM | 1190 | CA | SER | A | 161 | 47.811 | 47.561 | 81.576 | 1.00 | 39.94 | C |
| ATOM | 1191 | CB | SER | A | 161 | 47.753 | 46.991 | 82.999 | 1.00 | 41.19 | C |
| ATOM | 1192 | OG | SER | A | 161 | 49.020 | 47.070 | 83.643 | 1.00 | 44.11 | O |
| ATOM | 1193 | C | SER | A | 161 | 49.103 | 47.123 | 80.883 | 1.00 | 39.66 | C |
| ATOM | 1194 | O | SER | A | 161 | 49.994 | 47.939 | 80.655 | 1.00 | 38.58 | O |
| ATOM | 1195 | N | ARG | A | 162 | 49.191 | 45.841 | 80.522 | 1.00 | 39.42 | N |
| ATOM | 1196 | CA | ARG | A | 162 | 50.402 | 45.311 | 79.872 | 1.00 | 39.26 | C |
| ATOM | 1197 | CB | ARG | A | 162 | 50.263 | 43.803 | 79.619 | 1.00 | 39.95 | C |
| ATOM | 1198 | CG | ARG | A | 162 | 51.426 | 43.180 | 78.873 | 1.00 | 39.67 | C |
| ATOM | 1199 | CD | ARG | A | 162 | 51.167 | 41.696 | 78.616 | 1.00 | 42.82 | C |
| ATOM | 1200 | NE | ARG | A | 162 | 52.369 | 41.019 | 78.121 | 1.00 | 44.71 | N |
| ATOM | 1201 | CZ | ARG | A | 162 | 52.598 | 40.738 | 76.846 | 1.00 | 45.55 | C |
| ATOM | 1202 | NH1 | ARG | A | 162 | 51.702 | 41.066 | 75.923 | 1.00 | 47.22 | N |
| ATOM | 1203 | NH2 | ARG | A | 162 | 53.728 | 40.140 | 76.494 | 1.00 | 46.97 | N |
| ATOM | 1204 | C | ARG | A | 162 | 50.733 | 46.022 | 78.559 | 1.00 | 38.36 | C |
| ATOM | 1205 | O | ARG | A | 162 | 51.898 | 46.344 | 78.280 | 1.00 | 37.39 | O |
| ATOM | 1206 | N | ILE | A | 163 | 49.703 | 46.249 | 77.750 | 1.00 | 37.45 | N |
| ATOM | 1207 | CA | ILE | A | 163 | 49.861 | 46.939 | 76.477 | 1.00 | 35.75 | C |
| ATOM | 1208 | CB | ILE | A | 163 | 48.481 | 47.089 | 75.753 | 1.00 | 35.63 | C |
| ATOM | 1209 | CG2 | ILE | A | 163 | 48.547 | 48.164 | 74.662 | 1.00 | 33.84 | C |
| ATOM | 1210 | CG1 | ILE | A | 163 | 48.044 | 45.751 | 75.167 | 1.00 | 33.57 | C |
| ATOM | 1211 | CD1 | ILE | A | 163 | 46.599 | 45.750 | 74.685 | 1.00 | 33.13 | C |
| ATOM | 1212 | C | ILE | A | 163 | 50.462 | 48.336 | 76.702 | 1.00 | 35.50 | C |
| ATOM | 1213 | O | ILE | A | 163 | 51.354 | 48.753 | 75.970 | 1.00 | 36.19 | O |
| ATOM | 1214 | N | SER | A | 164 | 49.971 | 49.042 | 77.720 | 1.00 | 35.83 | N |
| ATOM | 1215 | CA | SER | A | 164 | 50.436 | 50.393 | 78.017 | 1.00 | 37.08 | C |
| ATOM | 1216 | CB | SER | A | 164 | 49.547 | 51.035 | 79.077 | 1.00 | 36.65 | C |
| ATOM | 1217 | OG | SER | A | 164 | 49.937 | 50.627 | 80.377 | 1.00 | 39.34 | O |
| ATOM | 1218 | C | SER | A | 164 | 51.892 | 50.419 | 78.488 | 1.00 | 38.71 | C |
| ATOM | 1219 | O | SER | A | 164 | 52.638 | 51.358 | 78.178 | 1.00 | 37.76 | O |
| ATOM | 1220 | N | ILE | A | 165 | 52.295 | 49.393 | 79.242 | 1.00 | 38.73 | N |
| ATOM | 1221 | CA | ILE | A | 165 | 53.667 | 49.317 | 79.725 | 1.00 | 38.60 | C |
| ATOM | 1222 | CB | ILE | A | 165 | 53.821 | 48.231 | 80.806 | 1.00 | 39.24 | C |
| ATOM | 1223 | CG2 | ILE | A | 165 | 55.295 | 48.029 | 81.150 | 1.00 | 38.68 | C |
| ATOM | 1224 | CG1 | ILE | A | 165 | 53.039 | 48.631 | 82.056 | 1.00 | 37.78 | C |
| ATOM | 1225 | CD1 | ILE | A | 165 | 53.052 | 47.566 | 83.139 | 1.00 | 38.24 | C |
| ATOM | 1226 | C | ILE | A | 165 | 54.579 | 49.002 | 78.539 | 1.00 | 39.87 | C |
| ATOM | 1227 | O | ILE | A | 165 | 55.635 | 49.614 | 78.367 | 1.00 | 39.97 | O |
| ATOM | 1228 | N | ARG | A | 166 | 54.152 | 48.067 | 77.697 | 1.00 | 41.98 | N |
| ATOM | 1229 | CA | ARG | A | 166 | 54.943 | 47.696 | 76.536 | 1.00 | 42.54 | C |
| ATOM | 1230 | CB | ARG | A | 166 | 54.344 | 46.444 | 75.876 | 1.00 | 44.21 | C |
| ATOM | 1231 | CG | ARG | A | 166 | 54.673 | 45.148 | 76.639 | 1.00 | 46.41 | C |
| ATOM | 1232 | CD | ARG | A | 166 | 53.851 | 43.963 | 76.178 | 1.00 | 49.11 | C |
| ATOM | 1233 | NE | ARG | A | 166 | 54.020 | 43.725 | 74.753 | 1.00 | 53.57 | N |
| ATOM | 1234 | CZ | ARG | A | 166 | 55.107 | 43.191 | 74.205 | 1.00 | 56.71 | C |
| ATOM | 1235 | NH1 | ARG | A | 166 | 56.137 | 42.820 | 74.972 | 1.00 | 56.69 | N |
| ATOM | 1236 | NH2 | ARG | A | 166 | 55.171 | 43.050 | 72.884 | 1.00 | 57.60 | N |
| ATOM | 1237 | C | ARG | A | 166 | 55.072 | 48.849 | 75.538 | 1.00 | 42.27 | C |
| ATOM | 1238 | O | ARG | A | 166 | 56.049 | 48.930 | 74.801 | 1.00 | 42.30 | O |
| ATOM | 1239 | N | MET | A | 167 | 54.094 | 49.747 | 75.518 | 1.00 | 42.25 | N |
| ATOM | 1240 | CA | MET | A | 167 | 54.151 | 50.892 | 74.616 | 1.00 | 42.49 | C |
| ATOM | 1241 | CB | MET | A | 167 | 52.822 | 51.655 | 74.638 | 1.00 | 43.63 | C |
| ATOM | 1242 | CG | MET | A | 167 | 52.754 | 52.880 | 73.720 | 1.00 | 44.39 | C |
| ATOM | 1243 | SD | MET | A | 167 | 52.866 | 52.468 | 71.986 | 1.00 | 45.85 | S |
| ATOM | 1244 | CE | MET | A | 167 | 53.217 | 54.046 | 71.247 | 1.00 | 45.14 | C |
| ATOM | 1245 | C | MET | A | 167 | 55.286 | 51.802 | 75.090 | 1.00 | 41.97 | C |
| ATOM | 1246 | O | MET | A | 167 | 56.140 | 52.212 | 74.303 | 1.00 | 41.69 | O |
| ATOM | 1247 | N | LEU | A | 168 | 55.296 | 52.105 | 76.385 | 1.00 | 42.02 | N |
| ATOM | 1248 | CA | LEU | A | 168 | 56.328 | 52.960 | 76.951 | 1.00 | 43.56 | C |
| ATOM | 1249 | CB | LEU | A | 168 | 56.116 | 53.133 | 78.460 | 1.00 | 42.10 | C |
| ATOM | 1250 | CG | LEU | A | 168 | 54.801 | 53.830 | 78.824 | 1.00 | 42.01 | C |
| ATOM | 1251 | CD1 | LEU | A | 168 | 54.850 | 54.385 | 80.234 | 1.00 | 40.37 | C |
| ATOM | 1252 | CD2 | LEU | A | 168 | 54.554 | 54.948 | 77.847 | 1.00 | 40.70 | C |
| ATOM | 1253 | C | LEU | A | 168 | 57.712 | 52.391 | 76.679 | 1.00 | 45.04 | C |
| ATOM | 1254 | O | LEU | A | 168 | 58.580 | 53.078 | 76.130 | 1.00 | 44.97 | O |
| ATOM | 1255 | N | ILE | A | 169 | 57.902 | 51.129 | 77.050 | 1.00 | 46.14 | N |
| ATOM | 1256 | CA | ILE | A | 169 | 59.178 | 50.461 | 76.853 | 1.00 | 47.74 | C |
| ATOM | 1257 | CB | ILE | A | 169 | 59.120 | 49.019 | 77.388 | 1.00 | 47.36 | C |
| ATOM | 1258 | CG2 | ILE | A | 169 | 60.278 | 48.194 | 76.837 | 1.00 | 45.27 | C |
| ATOM | 1259 | CG1 | ILE | A | 169 | 59.119 | 49.057 | 78.911 | 1.00 | 47.00 | C |
| ATOM | 1260 | CD1 | ILE | A | 169 | 59.051 | 47.706 | 79.552 | 1.00 | 47.60 | C |
| ATOM | 1261 | C | ILE | A | 169 | 59.634 | 50.444 | 75.397 | 1.00 | 49.37 | C |
| ATOM | 1262 | O | ILE | A | 169 | 60.791 | 50.749 | 75.098 | 1.00 | 48.89 | O |
| ATOM | 1263 | N | ASN | A | 170 | 58.736 | 50.088 | 74.488 | 1.00 | 50.68 | N |
| ATOM | 1264 | CA | ASN | A | 170 | 59.124 | 50.051 | 73.093 | 1.00 | 53.34 | C |
| ATOM | 1265 | CB | ASN | A | 170 | 58.029 | 49.408 | 72.233 | 1.00 | 54.68 | C |
| ATOM | 1266 | CG | ASN | A | 170 | 57.978 | 47.906 | 72.396 | 1.00 | 56.45 | C |
| ATOM | 1267 | OD1 | ASN | A | 170 | 59.019 | 47.258 | 72.544 | 1.00 | 57.55 | O |
| ATOM | 1268 | ND2 | ASN | A | 170 | 56.773 | 47.339 | 72.362 | 1.00 | 56.38 | N |
| ATOM | 1269 | C | ASN | A | 170 | 59.452 | 51.442 | 72.585 | 1.00 | 53.82 | C |
| ATOM | 1270 | O | ASN | A | 170 | 60.317 | 51.596 | 71.726 | 1.00 | 54.21 | O |
| ATOM | 1271 | N | GLN | A | 171 | 58.770 | 52.456 | 73.108 | 1.00 | 54.43 | N |
| ATOM | 1272 | CA | GLN | A | 171 | 59.048 | 53.819 | 72.672 | 1.00 | 56.01 | C |
| ATOM | 1273 | CB | GLN | A | 171 | 58.061 | 54.812 | 73.285 | 1.00 | 55.68 | C |
| ATOM | 1274 | CG | GLN | A | 171 | 56.797 | 55.017 | 72.464 | 1.00 | 56.18 | C |
| ATOM | 1275 | CD | GLN | A | 171 | 57.096 | 55.342 | 71.007 | 1.00 | 56.22 | C |
| ATOM | 1276 | OE1 | GLN | A | 171 | 57.174 | 54.450 | 70.156 | 1.00 | 56.11 | O |
| ATOM | 1277 | NE2 | GLN | A | 171 | 57.285 | 56.620 | 70.719 | 1.00 | 55.52 | N |
| ATOM | 1278 | C | GLN | A | 171 | 60.461 | 54.198 | 73.071 | 1.00 | 56.48 | C |
| ATOM | 1279 | O | GLN | A | 171 | 61.177 | 54.828 | 72.305 | 1.00 | 56.50 | O |
| ATOM | 1280 | N | HIS | A | 172 | 60.862 | 53.797 | 74.271 | 1.00 | 57.87 | N |
| ATOM | 1281 | CA | HIS | A | 172 | 62.197 | 54.105 | 74.754 | 1.00 | 59.21 | C |
| ATOM | 1282 | CB | HIS | A | 172 | 62.307 | 53.817 | 76.251 | 1.00 | 59.50 | C |
| ATOM | 1283 | CG | HIS | A | 172 | 63.543 | 54.384 | 76.876 | 1.00 | 61.79 | C |
| ATOM | 1284 | CD2 | HIS | A | 172 | 64.789 | 53.868 | 77.014 | 1.00 | 61.47 | C |
| ATOM | 1285 | ND1 | HIS | A | 172 | 63.606 | 55.671 | 77.367 | 1.00 | 61.58 | N |
| ATOM | 1286 | CE1 | HIS | A | 172 | 64.836 | 55.924 | 77.777 | 1.00 | 61.30 | C |
| ATOM | 1287 | NE2 | HIS | A | 172 | 65.573 | 54.847 | 77.572 | 1.00 | 62.10 | N |
| ATOM | 1288 | C | HIS | A | 172 | 63.277 | 53.319 | 74.004 | 1.00 | 59.58 | C |
| ATOM | 1289 | O | HIS | A | 172 | 64.190 | 53.911 | 73.423 | 1.00 | 59.56 | O |
| ATOM | 1290 | N | THR | A | 173 | 63.171 | 51.993 | 74.002 | 1.00 | 59.69 | N |
| ATOM | 1291 | CA | THR | A | 173 | 64.173 | 51.170 | 73.331 | 1.00 | 60.30 | C |
| ATOM | 1292 | CB | THR | A | 173 | 63.878 | 49.664 | 73.498 | 1.00 | 60.13 | C |
| ATOM | 1293 | OG1 | THR | A | 173 | 62.655 | 49.330 | 72.830 | 1.00 | 59.96 | O |
| ATOM | 1294 | CG2 | THR | A | 173 | 63.772 | 49.313 | 74.969 | 1.00 | 59.69 | C |
| ATOM | 1295 | C | THR | A | 173 | 64.347 | 51.469 | 71.843 | 1.00 | 60.88 | C |
| ATOM | 1296 | O | THR | A | 173 | 65.471 | 51.476 | 71.347 | 1.00 | 61.39 | O |
| ATOM | 1297 | N | LEU | A | 174 | 63.249 | 51.715 | 71.133 | 1.00 | 61.44 | N |
| ATOM | 1298 | CA | LEU | A | 174 | 63.328 | 52.007 | 69.703 | 1.00 | 62.34 | C |
| ATOM | 1299 | CB | LEU | A | 174 | 61.941 | 51.907 | 69.055 | 1.00 | 62.14 | C |
| ATOM | 1300 | CG | LEU | A | 174 | 61.257 | 50.537 | 68.989 | 1.00 | 62.00 | C |
| ATOM | 1301 | CD1 | LEU | A | 174 | 59.869 | 50.694 | 68.398 | 1.00 | 62.19 | C |
| ATOM | 1302 | CD2 | LEU | A | 174 | 62.074 | 49.580 | 68.139 | 1.00 | 61.24 | C |
| ATOM | 1303 | C | LEU | A | 174 | 63.924 | 53.386 | 69.410 | 1.00 | 63.33 | C |
| ATOM | 1304 | O | LEU | A | 174 | 64.753 | 53.533 | 68.510 | 1.00 | 63.36 | O |
| ATOM | 1305 | N | ILE | A | 175 | 63.497 | 54.397 | 70.161 | 1.00 | 64.61 | N |
| ATOM | 1306 | CA | ILE | A | 175 | 63.994 | 55.757 | 69.966 | 1.00 | 66.12 | C |
| ATOM | 1307 | CB | ILE | A | 175 | 63.177 | 56.777 | 70.801 | 1.00 | 65.69 | C |
| ATOM | 1308 | CG2 | ILE | A | 175 | 63.853 | 58.140 | 70.774 | 1.00 | 65.20 | C |
| ATOM | 1309 | CG1 | ILE | A | 175 | 61.754 | 56.885 | 70.250 | 1.00 | 65.86 | C |
| ATOM | 1310 | CD1 | ILE | A | 175 | 60.844 | 57.787 | 71.078 | 1.00 | 65.06 | C |
| ATOM | 1311 | C | ILE | A | 175 | 65.472 | 55.898 | 70.339 | 1.00 | 67.38 | C |
| ATOM | 1312 | O | ILE | A | 175 | 66.203 | 56.664 | 69.711 | 1.00 | 67.36 | O |
| ATOM | 1313 | N | PHE | A | 176 | 65.909 | 55.143 | 71.345 | 1.00 | 68.60 | N |
| ATOM | 1314 | CA | PHE | A | 176 | 67.292 | 55.215 | 71.813 | 1.00 | 70.15 | C |
| ATOM | 1315 | CB | PHE | A | 176 | 67.298 | 55.481 | 73.322 | 1.00 | 68.95 | C |
| ATOM | 1316 | CG | PHE | A | 176 | 66.451 | 56.655 | 73.726 | 1.00 | 68.38 | C |
| ATOM | 1317 | CD1 | PHE | A | 176 | 66.724 | 57.930 | 73.231 | 1.00 | 67.97 | C |
| ATOM | 1318 | CD2 | PHE | A | 176 | 65.353 | 56.483 | 74.561 | 1.00 | 68.47 | C |
| ATOM | 1319 | CE1 | PHE | A | 176 | 65.911 | 59.016 | 73.562 | 1.00 | 67.42 | C |
| ATOM | 1320 | CE2 | PHE | A | 176 | 64.532 | 57.562 | 74.899 | 1.00 | 67.75 | C |
| ATOM | 1321 | CZ | PHE | A | 176 | 64.812 | 58.830 | 74.395 | 1.00 | 67.47 | C |
| ATOM | 1322 | C | PHE | A | 176 | 68.150 | 53.989 | 71.495 | 1.00 | 71.80 | C |
| ATOM | 1323 | O | PHE | A | 176 | 69.139 | 53.728 | 72.180 | 1.00 | 71.95 | O |
| ATOM | 1324 | N | ASP | A | 177 | 67.772 | 53.258 | 70.450 | 1.00 | 73.85 | N |
| ATOM | 1325 | CA | ASP | A | 177 | 68.491 | 52.060 | 70.009 | 1.00 | 75.15 | C |
| ATOM | 1326 | CB | ASP | A | 177 | 68.407 | 51.941 | 68.478 | 1.00 | 76.36 | C |
| ATOM | 1327 | CG | ASP | A | 177 | 68.760 | 53.239 | 67.768 | 1.00 | 78.11 | C |
| ATOM | 1328 | OD1 | ASP | A | 177 | 69.918 | 53.697 | 67.896 | 1.00 | 79.02 | O |
| ATOM | 1329 | OD2 | ASP | A | 177 | 67.878 | 53.806 | 67.083 | 1.00 | 78.59 | O |
| ATOM | 1330 | C | ASP | A | 177 | 69.953 | 52.027 | 70.453 | 1.00 | 75.22 | C |
| ATOM | 1331 | O | ASP | A | 177 | 70.836 | 52.545 | 69.771 | 1.00 | 75.35 | O |
| ATOM | 1332 | N | PRO | A | 185 | 64.064 | 61.184 | 61.146 | 1.00 | 80.11 | N |
| ATOM | 1333 | CD | PRO | A | 185 | 65.266 | 61.644 | 60.423 | 1.00 | 80.43 | C |
| ATOM | 1334 | CA | PRO | A | 185 | 62.977 | 60.972 | 60.195 | 1.00 | 79.63 | C |
| ATOM | 1335 | CB | PRO | A | 185 | 63.721 | 60.426 | 58.987 | 1.00 | 80.23 | C |
| ATOM | 1336 | CG | PRO | A | 185 | 64.887 | 61.413 | 58.932 | 1.00 | 80.60 | C |
| ATOM | 1337 | C | PRO | A | 185 | 61.715 | 60.171 | 60.553 | 1.00 | 78.60 | C |
| ATOM | 1338 | O | PRO | A | 185 | 61.489 | 59.777 | 61.696 | 1.00 | 78.69 | O |
| ATOM | 1339 | N | LYS | A | 186 | 60.903 | 59.978 | 59.517 | 1.00 | 77.15 | N |
| ATOM | 1340 | CA | LYS | A | 186 | 59.603 | 59.310 | 59.513 | 1.00 | 75.35 | C |
| ATOM | 1341 | CB | LYS | A | 186 | 59.575 | 58.345 | 58.325 | 1.00 | 76.14 | C |
| ATOM | 1342 | CG | LYS | A | 186 | 59.913 | 59.111 | 57.051 | 1.00 | 77.53 | C |
| ATOM | 1343 | CD | LYS | A | 186 | 59.675 | 58.372 | 55.749 | 1.00 | 79.43 | C |
| ATOM | 1344 | CE | LYS | A | 186 | 60.154 | 59.252 | 54.586 | 1.00 | 80.43 | C |
| ATOM | 1345 | NZ | LYS | A | 186 | 59.928 | 58.670 | 53.231 | 1.00 | 81.12 | N |
| ATOM | 1346 | C | LYS | A | 186 | 58.944 | 58.685 | 60.752 | 1.00 | 73.56 | C |
| ATOM | 1347 | O | LYS | A | 186 | 57.866 | 59.125 | 61.132 | 1.00 | 73.57 | O |
| ATOM | 1348 | N | HIS | A | 187 | 59.541 | 57.689 | 61.395 | 1.00 | 71.25 | N |
| ATOM | 1349 | CA | HIS | A | 187 | 58.882 | 57.097 | 62.562 | 1.00 | 68.67 | C |
| ATOM | 1350 | CB | HIS | A | 187 | 58.957 | 55.572 | 62.494 | 1.00 | 68.40 | C |
| ATOM | 1351 | CG | HIS | A | 187 | 58.077 | 54.966 | 61.447 | 1.00 | 68.31 | C |
| ATOM | 1352 | CD2 | HIS | A | 187 | 57.200 | 53.939 | 61.510 | 1.00 | 68.03 | C |
| ATOM | 1353 | ND1 | HIS | A | 187 | 58.078 | 55.386 | 60.136 | 1.00 | 68.27 | N |
| ATOM | 1354 | CE1 | HIS | A | 187 | 57.240 | 54.639 | 59.437 | 1.00 | 67.91 | C |
| ATOM | 1355 | NE2 | HIS | A | 187 | 56.695 | 53.753 | 60.248 | 1.00 | 67.75 | N |
| ATOM | 1356 | C | HIS | A | 187 | 59.419 | 57.544 | 63.912 | 1.00 | 67.02 | C |
| ATOM | 1357 | O | HIS | A | 187 | 60.627 | 57.688 | 64.093 | 1.00 | 68.15 | O |
| ATOM | 1358 | N | ILE | A | 188 | 58.521 | 57.762 | 64.865 | 1.00 | 63.96 | N |
| ATOM | 1359 | CA | ILE | A | 188 | 58.946 | 58.142 | 66.199 | 1.00 | 61.19 | C |
| ATOM | 1360 | CB | ILE | A | 188 | 58.214 | 59.408 | 66.708 | 1.00 | 60.70 | C |
| ATOM | 1361 | CG2 | ILE | A | 188 | 58.604 | 59.699 | 68.155 | 1.00 | 57.89 | C |
| ATOM | 1362 | CG1 | ILE | A | 188 | 58.575 | 60.596 | 65.806 | 1.00 | 60.65 | C |
| ATOM | 1363 | CD1 | ILE | A | 188 | 58.144 | 61.955 | 66.341 | 1.00 | 62.30 | C |
| ATOM | 1364 | C | ILE | A | 188 | 58.680 | 56.954 | 67.110 | 1.00 | 59.86 | C |
| ATOM | 1365 | O | ILE | A | 188 | 57.687 | 56.904 | 67.839 | 1.00 | 59.47 | O |
| ATOM | 1366 | N | GLY | A | 189 | 59.593 | 55.991 | 67.046 | 1.00 | 58.20 | N |
| ATOM | 1367 | CA | GLY | A | 189 | 59.475 | 54.782 | 67.840 | 1.00 | 56.72 | C |
| ATOM | 1368 | C | GLY | A | 189 | 58.551 | 53.833 | 67.102 | 1.00 | 55.26 | C |
| ATOM | 1369 | O | GLY | A | 189 | 58.835 | 53.428 | 65.972 | 1.00 | 54.86 | O |
| ATOM | 1370 | N | SER | A | 190 | 57.439 | 53.486 | 67.735 | 1.00 | 52.94 | N |
| ATOM | 1371 | CA | SER | A | 190 | 56.468 | 52.603 | 67.113 | 1.00 | 51.69 | C |
| ATOM | 1372 | CB | SER | A | 190 | 55.866 | 51.667 | 68.155 | 1.00 | 51.14 | C |
| ATOM | 1373 | OG | SER | A | 190 | 55.053 | 52.393 | 69.063 | 1.00 | 51.58 | O |
| ATOM | 1374 | C | SER | A | 190 | 55.357 | 53.450 | 66.489 | 1.00 | 50.72 | C |
| ATOM | 1375 | O | SER | A | 190 | 54.373 | 52.923 | 65.977 | 1.00 | 49.23 | O |
| ATOM | 1376 | N | ILE | A | 191 | 55.520 | 54.769 | 66.530 | 1.00 | 49.60 | N |
| ATOM | 1377 | CA | ILE | A | 191 | 54.505 | 55.663 | 65.984 | 1.00 | 48.38 | C |
| ATOM | 1378 | CB | ILE | A | 191 | 54.293 | 56.876 | 66.896 | 1.00 | 48.86 | C |
| ATOM | 1379 | CG2 | ILE | A | 191 | 53.250 | 57.809 | 66.292 | 1.00 | 47.93 | C |
| ATOM | 1380 | CG1 | ILE | A | 191 | 53.863 | 56.397 | 68.278 | 1.00 | 49.06 | C |
| ATOM | 1381 | CD1 | ILE | A | 191 | 53.637 | 57.506 | 69.254 | 1.00 | 50.18 | C |
| ATOM | 1382 | C | ILE | A | 191 | 54.785 | 56.180 | 64.586 | 1.00 | 47.29 | C |
| ATOM | 1383 | O | ILE | A | 191 | 55.873 | 56.668 | 64.293 | 1.00 | 47.42 | O |
| ATOM | 1384 | N | ASP | A | 192 | 53.791 | 56.049 | 63.720 | 1.00 | 45.89 | N |
| ATOM | 1385 | CA | ASP | A | 192 | 53.898 | 56.552 | 62.365 | 1.00 | 45.79 | C |
| ATOM | 1386 | CB | ASP | A | 192 | 53.385 | 55.539 | 61.350 | 1.00 | 45.24 | C |
| ATOM | 1387 | CG | ASP | A | 192 | 53.685 | 55.950 | 59.926 | 1.00 | 43.99 | C |
| ATOM | 1388 | OD1 | ASP | A | 192 | 53.873 | 57.159 | 59.685 | 1.00 | 43.91 | O |
| ATOM | 1389 | OD2 | ASP | A | 192 | 53.721 | 55.074 | 59.048 | 1.00 | 43.31 | O |
| ATOM | 1390 | C | ASP | A | 192 | 53.007 | 57.787 | 62.340 | 1.00 | 46.75 | C |
| ATOM | 1391 | O | ASP | A | 192 | 51.790 | 57.692 | 62.519 | 1.00 | 44.95 | O |
| ATOM | 1392 | N | PRO | A | 193 | 53.608 | 58.970 | 62.153 | 1.00 | 47.99 | N |
| ATOM | 1393 | CD | PRO | A | 193 | 55.053 | 59.231 | 62.031 | 1.00 | 47.99 | C |
| ATOM | 1394 | CA | PRO | A | 193 | 52.845 | 60.222 | 62.112 | 1.00 | 48.63 | C |
| ATOM | 1395 | CB | PRO | A | 193 | 53.927 | 61.285 | 62.268 | 1.00 | 49.00 | C |
| ATOM | 1396 | CG | PRO | A | 193 | 55.083 | 60.671 | 61.544 | 1.00 | 48.48 | C |
| ATOM | 1397 | C | PRO | A | 193 | 52.071 | 60.356 | 60.808 | 1.00 | 48.61 | C |
| ATOM | 1398 | O | PRO | A | 193 | 51.200 | 61.202 | 60.691 | 1.00 | 49.13 | O |
| ATOM | 1399 | N | ASN | A | 194 | 52.404 | 59.509 | 59.838 | 1.00 | 49.20 | N |
| ATOM | 1400 | CA | ASN | A | 194 | 51.728 | 59.503 | 58.542 | 1.00 | 50.54 | C |
| ATOM | 1401 | CB | ASN | A | 194 | 52.585 | 60.178 | 57.472 | 1.00 | 53.66 | C |
| ATOM | 1402 | CG | ASN | A | 194 | 52.365 | 61.674 | 57.421 | 1.00 | 57.34 | C |
| ATOM | 1403 | OD1 | ASN | A | 194 | 52.882 | 62.426 | 58.258 | 1.00 | 58.28 | O |
| ATOM | 1404 | ND2 | ASN | A | 194 | 51.573 | 62.116 | 56.446 | 1.00 | 59.48 | N |
| ATOM | 1405 | C | ASN | A | 194 | 51.359 | 58.101 | 58.070 | 1.00 | 48.82 | C |
| ATOM | 1406 | O | ASN | A | 194 | 51.648 | 57.723 | 56.932 | 1.00 | 48.30 | O |
| ATOM | 1407 | N | CYS | A | 195 | 50.705 | 57.346 | 58.949 | 1.00 | 47.26 | N |
| ATOM | 1408 | CA | CYS | A | 195 | 50.282 | 55.988 | 58.652 | 1.00 | 45.66 | C |
| ATOM | 1409 | CB | CYS | A | 195 | 49.771 | 55.325 | 59.929 | 1.00 | 45.60 | C |
| ATOM | 1410 | SG | CYS | A | 195 | 49.082 | 53.691 | 59.681 | 1.00 | 43.70 | S |
| ATOM | 1411 | C | CYS | A | 195 | 49.188 | 55.957 | 57.584 | 1.00 | 45.12 | C |
| ATOM | 1412 | O | CYS | A | 195 | 48.129 | 56.572 | 57.744 | 1.00 | 44.77 | O |
| ATOM | 1413 | N | ASN | A | 196 | 49.456 | 55.244 | 56.495 | 1.00 | 45.08 | N |
| ATOM | 1414 | CA | ASN | A | 196 | 48.508 | 55.100 | 55.396 | 1.00 | 45.31 | C |
| ATOM | 1415 | CB | ASN | A | 196 | 49.272 | 54.922 | 54.078 | 1.00 | 46.87 | C |
| ATOM | 1416 | CG | ASN | A | 196 | 48.353 | 54.755 | 52.869 | 1.00 | 48.10 | C |
| ATOM | 1417 | OD1 | ASN | A | 196 | 47.140 | 54.885 | 52.969 | 1.00 | 50.88 | O |
| ATOM | 1418 | ND2 | ASN | A | 196 | 48.942 | 54.473 | 51.719 | 1.00 | 48.57 | N |
| ATOM | 1419 | C | ASN | A | 196 | 47.666 | 53.862 | 55.706 | 1.00 | 44.61 | C |
| ATOM | 1420 | O | ASN | A | 196 | 48.054 | 52.736 | 55.375 | 1.00 | 44.30 | O |
| ATOM | 1421 | N | VAL | A | 197 | 46.521 | 54.088 | 56.349 | 1.00 | 43.88 | N |
| ATOM | 1422 | CA | VAL | A | 197 | 45.623 | 53.017 | 56.761 | 1.00 | 43.27 | C |
| ATOM | 1423 | CB | VAL | A | 197 | 44.252 | 53.586 | 57.220 | 1.00 | 41.89 | C |
| ATOM | 1424 | CG1 | VAL | A | 197 | 43.332 | 52.463 | 57.677 | 1.00 | 40.81 | C |
| ATOM | 1425 | CG2 | VAL | A | 197 | 44.468 | 54.549 | 58.371 | 1.00 | 40.98 | C |
| ATOM | 1426 | C | VAL | A | 197 | 45.418 | 51.944 | 55.701 | 1.00 | 43.54 | C |
| ATOM | 1427 | O | VAL | A | 197 | 45.614 | 50.760 | 55.978 | 1.00 | 44.44 | O |
| ATOM | 1428 | N | SER | A | 198 | 45.039 | 52.352 | 54.495 | 1.00 | 44.25 | N |
| ATOM | 1429 | CA | SER | A | 198 | 44.819 | 51.404 | 53.411 | 1.00 | 45.08 | C |
| ATOM | 1430 | CB | SER | A | 198 | 44.478 | 52.144 | 52.116 | 1.00 | 45.81 | C |
| ATOM | 1431 | OG | SER | A | 198 | 43.073 | 52.325 | 51.998 | 1.00 | 48.95 | O |
| ATOM | 1432 | C | SER | A | 198 | 46.002 | 50.477 | 53.168 | 1.00 | 44.52 | C |
| ATOM | 1433 | O | SER | A | 198 | 45.829 | 49.300 | 52.838 | 1.00 | 44.83 | O |
| ATOM | 1434 | N | GLU | A | 199 | 47.211 | 50.998 | 53.326 | 1.00 | 45.37 | N |
| ATOM | 1435 | CA | GLU | A | 199 | 48.392 | 50.176 | 53.105 | 1.00 | 45.78 | C |
| ATOM | 1436 | CB | GLU | A | 199 | 49.659 | 51.045 | 53.086 | 1.00 | 48.79 | C |
| ATOM | 1437 | CG | GLU | A | 199 | 50.935 | 50.286 | 52.698 | 1.00 | 53.46 | C |
| ATOM | 1438 | CD | GLU | A | 199 | 50.793 | 49.537 | 51.380 | 1.00 | 56.53 | C |
| ATOM | 1439 | OE1 | GLU | A | 199 | 50.379 | 50.171 | 50.378 | 1.00 | 59.09 | O |
| ATOM | 1440 | OE2 | GLU | A | 199 | 51.094 | 48.318 | 51.347 | 1.00 | 58.88 | O |
| ATOM | 1441 | C | GLU | A | 199 | 48.483 | 49.103 | 54.196 | 1.00 | 44.04 | C |
| ATOM | 1442 | O | GLU | A | 199 | 48.777 | 47.935 | 53.909 | 1.00 | 42.77 | O |
| ATOM | 1443 | N | VAL | A | 200 | 48.221 | 49.488 | 55.443 | 1.00 | 41.54 | N |
| ATOM | 1444 | CA | VAL | A | 200 | 48.262 | 48.511 | 56.518 | 1.00 | 40.70 | C |
| ATOM | 1445 | CB | VAL | A | 200 | 48.036 | 49.162 | 57.880 | 1.00 | 40.42 | C |
| ATOM | 1446 | CG1 | VAL | A | 200 | 48.237 | 48.135 | 58.975 | 1.00 | 39.69 | C |
| ATOM | 1447 | CG2 | VAL | A | 200 | 49.000 | 50.307 | 58.059 | 1.00 | 40.79 | C |
| ATOM | 1448 | C | VAL | A | 200 | 47.199 | 47.442 | 56.269 | 1.00 | 40.19 | C |
| ATOM | 1449 | O | VAL | A | 200 | 47.453 | 46.253 | 56.490 | 1.00 | 39.74 | O |
| ATOM | 1450 | N | VAL | A | 201 | 46.025 | 47.864 | 55.790 | 1.00 | 38.69 | N |
| ATOM | 1451 | CA | VAL | A | 201 | 44.942 | 46.936 | 55.478 | 1.00 | 38.31 | C |
| ATOM | 1452 | CB | VAL | A | 201 | 43.711 | 47.669 | 54.835 | 1.00 | 38.33 | C |
| ATOM | 1453 | CG1 | VAL | A | 201 | 42.691 | 46.647 | 54.330 | 1.00 | 35.62 | C |
| ATOM | 1454 | CG2 | VAL | A | 201 | 43.059 | 48.593 | 55.840 | 1.00 | 36.95 | C |
| ATOM | 1455 | C | VAL | A | 201 | 45.447 | 45.892 | 54.477 | 1.00 | 38.54 | C |
| ATOM | 1456 | O | VAL | A | 201 | 45.308 | 44.683 | 54.693 | 1.00 | 37.16 | O |
| ATOM | 1457 | N | LYS | A | 202 | 46.023 | 46.384 | 53.379 | 1.00 | 40.29 | N |
| ATOM | 1458 | CA | LYS | A | 202 | 46.577 | 45.547 | 52.304 | 1.00 | 41.92 | C |
| ATOM | 1459 | CB | LYS | A | 202 | 47.366 | 46.400 | 51.310 | 1.00 | 44.01 | C |
| ATOM | 1460 | CG | LYS | A | 202 | 46.828 | 46.459 | 49.910 | 1.00 | 46.31 | C |
| ATOM | 1461 | CD | LYS | A | 202 | 46.149 | 47.802 | 49.695 | 1.00 | 50.54 | C |
| ATOM | 1462 | CE | LYS | A | 202 | 46.829 | 48.621 | 48.604 | 1.00 | 52.18 | C |
| ATOM | 1463 | NZ | LYS | A | 202 | 46.538 | 50.087 | 48.766 | 1.00 | 54.41 | N |
| ATOM | 1464 | C | LYS | A | 202 | 47.539 | 44.495 | 52.844 | 1.00 | 41.44 | C |
| ATOM | 1465 | O | LYS | A | 202 | 47.462 | 43.320 | 52.461 | 1.00 | 41.20 | O |
| ATOM | 1466 | N | ASP | A | 203 | 48.454 | 44.938 | 53.711 | 1.00 | 40.64 | N |
| ATOM | 1467 | CA | ASP | A | 203 | 49.459 | 44.061 | 54.303 | 1.00 | 41.71 | C |
| ATOM | 1468 | CB | ASP | A | 203 | 50.483 | 44.858 | 55.129 | 1.00 | 44.04 | C |
| ATOM | 1469 | CG | ASP | A | 203 | 51.300 | 45.849 | 54.296 | 1.00 | 46.35 | C |
| ATOM | 1470 | OD1 | ASP | A | 203 | 51.340 | 45.742 | 53.047 | 1.00 | 47.13 | O |
| ATOM | 1471 | OD2 | ASP | A | 203 | 51.926 | 46.742 | 54.915 | 1.00 | 47.79 | O |
| ATOM | 1472 | C | ASP | A | 203 | 48.858 | 42.981 | 55.198 | 1.00 | 41.15 | C |
| ATOM | 1473 | O | ASP | A | 203 | 49.186 | 41.802 | 55.049 | 1.00 | 41.47 | O |
| ATOM | 1474 | N | ALA | A | 204 | 48.008 | 43.383 | 56.145 | 1.00 | 40.70 | N |
| ATOM | 1475 | CA | ALA | A | 204 | 47.361 | 42.426 | 57.045 | 1.00 | 39.98 | C |
| ATOM | 1476 | CB | ALA | A | 204 | 46.393 | 43.144 | 57.965 | 1.00 | 40.74 | C |
| ATOM | 1477 | C | ALA | A | 204 | 46.615 | 41.420 | 56.175 | 1.00 | 39.69 | C |
| ATOM | 1478 | O | ALA | A | 204 | 46.607 | 40.226 | 56.441 | 1.00 | 37.86 | O |
| ATOM | 1479 | N | TYR | A | 205 | 46.001 | 41.924 | 55.113 | 1.00 | 40.28 | N |
| ATOM | 1480 | CA | TYR | A | 205 | 45.272 | 41.064 | 54.202 | 1.00 | 41.96 | C |
| ATOM | 1481 | CB | TYR | A | 205 | 44.564 | 41.889 | 53.132 | 1.00 | 42.01 | C |
| ATOM | 1482 | CG | TYR | A | 205 | 44.002 | 41.017 | 52.041 | 1.00 | 43.13 | C |
| ATOM | 1483 | CD1 | TYR | A | 205 | 42.895 | 40.201 | 52.285 | 1.00 | 43.69 | C |
| ATOM | 1484 | CE1 | TYR | A | 205 | 42.412 | 39.333 | 51.317 | 1.00 | 43.44 | C |
| ATOM | 1485 | CD2 | TYR | A | 205 | 44.618 | 40.948 | 50.787 | 1.00 | 43.39 | C |
| ATOM | 1486 | CE2 | TYR | A | 205 | 44.147 | 40.080 | 49.806 | 1.00 | 44.21 | C |
| ATOM | 1487 | CZ | TYR | A | 205 | 43.039 | 39.273 | 50.083 | 1.00 | 44.69 | C |
| ATOM | 1488 | OH | TYR | A | 205 | 42.555 | 38.409 | 49.136 | 1.00 | 44.26 | O |
| ATOM | 1489 | C | TYR | A | 205 | 46.194 | 40.052 | 53.519 | 1.00 | 42.37 | C |
| ATOM | 1490 | O | TYR | A | 205 | 45.930 | 38.849 | 53.557 | 1.00 | 42.28 | O |
| ATOM | 1491 | N | ASP | A | 206 | 47.264 | 40.544 | 52.889 | 1.00 | 42.41 | N |
| ATOM | 1492 | CA | ASP | A | 206 | 48.214 | 39.673 | 52.194 | 1.00 | 42.86 | C |
| ATOM | 1493 | CB | ASP | A | 206 | 49.402 | 40.482 | 51.638 | 1.00 | 43.51 | C |
| ATOM | 1494 | CG | ASP | A | 206 | 49.033 | 41.346 | 50.429 | 1.00 | 43.92 | C |
| ATOM | 1495 | OD1 | ASP | A | 206 | 48.130 | 40.956 | 49.658 | 1.00 | 44.03 | O |
| ATOM | 1496 | OD2 | ASP | A | 206 | 49.667 | 42.407 | 50.237 | 1.00 | 43.01 | O |
| ATOM | 1497 | C | ASP | A | 206 | 48.740 | 38.571 | 53.124 | 1.00 | 43.08 | C |
| ATOM | 1498 | O | ASP | A | 206 | 48.835 | 37.400 | 52.743 | 1.00 | 42.00 | O |
| ATOM | 1499 | N | MET | A | 207 | 49.073 | 38.944 | 54.351 | 1.00 | 43.08 | N |
| ATOM | 1500 | CA | MET | A | 207 | 49.581 | 37.969 | 55.298 | 1.00 | 43.72 | C |
| ATOM | 1501 | CB | MET | A | 207 | 50.013 | 38.648 | 56.592 | 1.00 | 43.58 | C |
| ATOM | 1502 | CG | MET | A | 207 | 50.748 | 37.718 | 57.537 | 1.00 | 46.49 | C |
| ATOM | 1503 | SD | MET | A | 207 | 52.226 | 36.951 | 56.749 | 1.00 | 49.00 | S |
| ATOM | 1504 | CE | MET | A | 207 | 51.946 | 35.214 | 57.113 | 1.00 | 47.13 | C |
| ATOM | 1505 | C | MET | A | 207 | 48.513 | 36.939 | 55.616 | 1.00 | 44.02 | C |
| ATOM | 1506 | O | MET | A | 207 | 48.806 | 35.746 | 55.718 | 1.00 | 44.20 | O |
| ATOM | 1507 | N | ALA | A | 208 | 47.276 | 37.400 | 55.793 | 1.00 | 44.02 | N |
| ATOM | 1508 | CA | ALA | A | 208 | 46.191 | 36.490 | 56.112 | 1.00 | 43.76 | C |
| ATOM | 1509 | CB | ALA | A | 208 | 44.919 | 37.269 | 56.449 | 1.00 | 43.78 | C |
| ATOM | 1510 | C | ALA | A | 208 | 45.944 | 35.549 | 54.938 | 1.00 | 43.77 | C |
| ATOM | 1511 | O | ALA | A | 208 | 45.783 | 34.351 | 55.131 | 1.00 | 42.78 | O |
| ATOM | 1512 | N | LYS | A | 209 | 45.922 | 36.103 | 53.727 | 1.00 | 45.19 | N |
| ATOM | 1513 | CA | LYS | A | 209 | 45.704 | 35.323 | 52.505 | 1.00 | 48.50 | C |
| ATOM | 1514 | CB | LYS | A | 209 | 45.844 | 36.226 | 51.280 | 1.00 | 48.69 | C |
| ATOM | 1515 | CG | LYS | A | 209 | 45.695 | 35.514 | 49.943 | 1.00 | 50.05 | C |
| ATOM | 1516 | CD | LYS | A | 209 | 45.786 | 36.512 | 48.802 | 1.00 | 51.15 | C |
| ATOM | 1517 | CE | LYS | A | 209 | 45.464 | 35.864 | 47.473 | 1.00 | 53.41 | C |
| ATOM | 1518 | NZ | LYS | A | 209 | 45.340 | 36.889 | 46.391 | 1.00 | 55.73 | N |
| ATOM | 1519 | C | LYS | A | 209 | 46.690 | 34.159 | 52.388 | 1.00 | 50.01 | C |
| ATOM | 1520 | O | LYS | A | 209 | 46.309 | 33.020 | 52.099 | 1.00 | 49.71 | O |
| ATOM | 1521 | N | LEU | A | 210 | 47.959 | 34.465 | 52.624 | 1.00 | 52.01 | N |
| ATOM | 1522 | CA | LEU | A | 210 | 49.022 | 33.480 | 52.555 | 1.00 | 53.37 | C |
| ATOM | 1523 | CB | LEU | A | 210 | 50.356 | 34.142 | 52.932 | 1.00 | 55.17 | C |
| ATOM | 1524 | CG | LEU | A | 210 | 51.650 | 33.392 | 52.614 | 1.00 | 57.21 | C |
| ATOM | 1525 | CD1 | LEU | A | 210 | 52.834 | 34.343 | 52.763 | 1.00 | 58.45 | C |
| ATOM | 1526 | CD2 | LEU | A | 210 | 51.802 | 32.196 | 53.544 | 1.00 | 57.83 | C |
| ATOM | 1527 | C | LEU | A | 210 | 48.712 | 32.300 | 53.472 | 1.00 | 53.51 | C |
| ATOM | 1528 | O | LEU | A | 210 | 48.808 | 31.145 | 53.052 | 1.00 | 53.95 | O |
| ATOM | 1529 | N | LEU | A | 211 | 48.330 | 32.587 | 54.714 | 1.00 | 52.95 | N |
| ATOM | 1530 | CA | LEU | A | 211 | 48.006 | 31.528 | 55.669 | 1.00 | 52.51 | C |
| ATOM | 1531 | CB | LEU | A | 211 | 47.764 | 32.098 | 57.063 | 1.00 | 51.27 | C |
| ATOM | 1532 | CG | LEU | A | 211 | 48.918 | 32.816 | 57.754 | 1.00 | 50.18 | C |
| ATOM | 1533 | CD1 | LEU | A | 211 | 48.486 | 33.263 | 59.132 | 1.00 | 48.59 | C |
| ATOM | 1534 | CD2 | LEU | A | 211 | 50.095 | 31.881 | 57.854 | 1.00 | 50.02 | C |
| ATOM | 1535 | C | LEU | A | 211 | 46.757 | 30.770 | 55.245 | 1.00 | 53.78 | C |
| ATOM | 1536 | O | LEU | A | 211 | 46.681 | 29.538 | 55.356 | 1.00 | 53.54 | O |
| ATOM | 1537 | N | CYS | A | 212 | 45.769 | 31.518 | 54.771 | 1.00 | 54.77 | N |
| ATOM | 1538 | CA | CYS | A | 212 | 44.516 | 30.927 | 54.331 | 1.00 | 55.44 | C |
| ATOM | 1539 | CB | CYS | A | 212 | 43.511 | 32.030 | 53.971 | 1.00 | 54.09 | C |
| ATOM | 1540 | SG | CYS | A | 212 | 41.801 | 31.462 | 53.770 | 1.00 | 53.76 | S |
| ATOM | 1541 | C | CYS | A | 212 | 44.760 | 30.001 | 53.131 | 1.00 | 55.72 | C |
| ATOM | 1542 | O | CYS | A | 212 | 44.225 | 28.892 | 53.080 | 1.00 | 53.78 | O |
| ATOM | 1543 | N | ASP | A | 213 | 45.575 | 30.455 | 52.178 | 1.00 | 57.12 | N |
| ATOM | 1544 | CA | ASP | A | 213 | 45.882 | 29.653 | 50.996 | 1.00 | 58.37 | C |
| ATOM | 1545 | CB | ASP | A | 213 | 46.754 | 30.442 | 50.010 | 1.00 | 56.64 | C |
| ATOM | 1546 | CG | ASP | A | 213 | 45.950 | 31.436 | 49.180 | 1.00 | 56.82 | C |
| ATOM | 1547 | CD1 | ASP | A | 213 | 44.746 | 31.189 | 48.944 | 1.00 | 55.30 | O |
| ATOM | 1548 | OD2 | ASP | A | 213 | 46.524 | 32.456 | 48.738 | 1.00 | 57.15 | O |
| ATOM | 1549 | C | ASP | A | 213 | 46.574 | 28.333 | 51.353 | 1.00 | 59.83 | C |
| ATOM | 1550 | O | ASP | A | 213 | 46.248 | 27.287 | 50.804 | 1.00 | 60.14 | O |
| ATOM | 1551 | N | LYS | A | 214 | 47.520 | 28.380 | 52.280 | 1.00 | 61.72 | N |
| ATOM | 1552 | CA | LYS | A | 214 | 48.234 | 27.177 | 52.679 | 1.00 | 64.62 | C |
| ATOM | 1553 | CB | LYS | A | 214 | 49.379 | 27.546 | 53.629 | 1.00 | 65.58 | C |
| ATOM | 1554 | CG | LYS | A | 214 | 49.735 | 26.454 | 54.619 | 1.00 | 67.72 | C |
| ATOM | 1555 | CD | LYS | A | 214 | 50.949 | 26.804 | 55.479 | 1.00 | 68.97 | C |
| ATOM | 1556 | CE | LYS | A | 214 | 51.593 | 25.517 | 56.000 | 1.00 | 69.47 | C |
| ATOM | 1557 | NZ | LYS | A | 214 | 52.512 | 25.695 | 57.159 | 1.00 | 69.27 | N |
| ATOM | 1558 | C | LYS | A | 214 | 47.327 | 26.140 | 53.341 | 1.00 | 66.07 | C |
| ATOM | 1559 | O | LYS | A | 214 | 47.418 | 24.944 | 53.061 | 1.00 | 66.66 | O |
| ATOM | 1560 | N | TYR | A | 215 | 46.446 | 26.604 | 54.213 | 1.00 | 67.80 | N |
| ATOM | 1561 | CA | TYR | A | 215 | 45.557 | 25.716 | 54.938 | 1.00 | 68.64 | C |
| ATOM | 1562 | CB | TYR | A | 215 | 45.154 | 26.379 | 56.251 | 1.00 | 71.58 | C |
| ATOM | 1563 | CG | TYR | A | 215 | 44.696 | 25.402 | 57.301 | 1.00 | 74.97 | C |
| ATOM | 1564 | CD1 | TYR | A | 215 | 45.621 | 24.709 | 58.081 | 1.00 | 76.37 | C |
| ATOM | 1565 | CE1 | TYR | A | 215 | 45.207 | 23.782 | 59.035 | 1.00 | 77.82 | C |
| ATOM | 1566 | CD2 | TYR | A | 215 | 43.334 | 25.146 | 57.499 | 1.00 | 76.48 | C |
| ATOM | 1567 | CE2 | TYR | A | 215 | 42.906 | 24.220 | 58.446 | 1.00 | 77.95 | C |
| ATOM | 1568 | CZ | TYR | A | 215 | 43.846 | 23.542 | 59.210 | 1.00 | 78.67 | C |
| ATOM | 1569 | OH | TYR | A | 215 | 43.421 | 22.624 | 60.143 | 1.00 | 79.58 | O |
| ATOM | 1570 | C | TYR | A | 215 | 44.298 | 25.285 | 54.181 | 1.00 | 68.35 | C |
| ATOM | 1571 | O | TYR | A | 215 | 43.851 | 24.149 | 54.323 | 1.00 | 68.30 | O |
| ATOM | 1572 | N | TYR | A | 216 | 43.723 | 26.183 | 53.385 | 1.00 | 67.84 | N |
| ATOM | 1573 | CA | TYR | A | 216 | 42.503 | 25.869 | 52.636 | 1.00 | 67.61 | C |
| ATOM | 1574 | CB | TYR | A | 216 | 41.384 | 26.868 | 52.988 | 1.00 | 66.87 | C |
| ATOM | 1575 | CG | TYR | A | 216 | 40.983 | 26.918 | 54.451 | 1.00 | 65.95 | C |
| ATOM | 1576 | CD1 | TYR | A | 216 | 41.381 | 27.977 | 55.270 | 1.00 | 65.50 | C |
| ATOM | 1577 | CE1 | TYR | A | 216 | 40.986 | 28.042 | 56.614 | 1.00 | 65.62 | C |
| ATOM | 1578 | CD2 | TYR | A | 216 | 40.183 | 25.919 | 55.012 | 1.00 | 65.97 | C |
| ATOM | 1579 | CE2 | TYR | A | 216 | 39.783 | 25.971 | 56.355 | 1.00 | 65.47 | C |
| ATOM | 1580 | CZ | TYR | A | 216 | 40.186 | 27.034 | 57.150 | 1.00 | 66.02 | C |
| ATOM | 1581 | OH | TYR | A | 216 | 39.785 | 27.090 | 58.474 | 1.00 | 65.03 | O |
| ATOM | 1582 | C | TYR | A | 216 | 42.681 | 25.847 | 51.113 | 1.00 | 68.49 | C |
| ATOM | 1583 | O | TYR | A | 216 | 41.781 | 25.438 | 50.382 | 1.00 | 68.54 | O |
| ATOM | 1584 | N | MET | A | 217 | 43.833 | 26.299 | 50.633 | 1.00 | 69.71 | N |
| ATOM | 1585 | CA | MET | A | 217 | 44.113 | 26.327 | 49.198 | 1.00 | 71.53 | C |
| ATOM | 1586 | CB | MET | A | 217 | 44.389 | 24.908 | 48.694 | 1.00 | 74.55 | C |
| ATOM | 1587 | CG | MET | A | 217 | 45.692 | 24.351 | 49.247 | 1.00 | 78.72 | C |
| ATOM | 1588 | SD | MET | A | 217 | 46.092 | 22.693 | 48.689 | 1.00 | 84.07 | S |
| ATOM | 1589 | CE | MET | A | 217 | 47.804 | 22.549 | 49.287 | 1.00 | 82.99 | C |
| ATOM | 1590 | C | MET | A | 217 | 43.000 | 26.991 | 48.390 | 1.00 | 70.47 | C |
| ATOM | 1591 | O | MET | A | 217 | 42.597 | 26.531 | 47.317 | 1.00 | 70.80 | O |
| ATOM | 1592 | N | ALA | A | 218 | 42.525 | 28.097 | 48.942 | 1.00 | 68.25 | N |
| ATOM | 1593 | CA | ALA | A | 218 | 41.480 | 28.913 | 48.351 | 1.00 | 65.39 | C |
| ATOM | 1594 | CB | ALA | A | 218 | 40.175 | 28.135 | 48.259 | 1.00 | 65.89 | C |
| ATOM | 1595 | C | ALA | A | 218 | 41.352 | 30.069 | 49.331 | 1.00 | 63.02 | C |
| ATOM | 1596 | O | ALA | A | 218 | 41.432 | 29.876 | 50.547 | 1.00 | 62.95 | O |
| ATOM | 1597 | N | SER | A | 219 | 41.182 | 31.272 | 48.810 | 1.00 | 59.97 | N |
| ATOM | 1598 | CA | SER | A | 219 | 41.077 | 32.421 | 49.675 | 1.00 | 56.79 | C |
| ATOM | 1599 | CB | SER | A | 219 | 42.464 | 32.990 | 49.988 | 1.00 | 57.43 | C |
| ATOM | 1600 | OG | SER | A | 219 | 43.048 | 33.588 | 48.840 | 1.00 | 57.10 | O |
| ATOM | 1601 | C | SER | A | 219 | 40.236 | 33.495 | 49.031 | 1.00 | 55.44 | C |
| ATOM | 1602 | O | SER | A | 219 | 40.212 | 33.655 | 47.814 | 1.00 | 55.02 | O |
| ATOM | 1603 | N | PRO | A | 220 | 39.514 | 34.249 | 49.856 | 1.00 | 54.22 | N |
| ATOM | 1604 | CD | PRO | A | 220 | 39.318 | 34.038 | 51.302 | 1.00 | 52.97 | C |
| ATOM | 1605 | CA | PRO | A | 220 | 38.665 | 35.325 | 49.350 | 1.00 | 52.28 | C |
| ATOM | 1606 | CB | PRO | A | 220 | 37.751 | 35.609 | 50.541 | 1.00 | 52.64 | C |
| ATOM | 1607 | CG | PRO | A | 220 | 38.639 | 35.309 | 51.716 | 1.00 | 53.22 | C |
| ATOM | 1608 | C | PRO | A | 220 | 39.517 | 36.534 | 48.943 | 1.00 | 50.77 | C |
| ATOM | 1609 | O | PRO | A | 220 | 40.591 | 36.744 | 49.489 | 1.00 | 49.59 | O |
| ATOM | 1610 | N | ASP | A | 221 | 39.040 | 37.316 | 47.982 | 1.00 | 49.87 | N |
| ATOM | 1611 | CA | ASP | A | 221 | 39.761 | 38.501 | 47.530 | 1.00 | 49.97 | C |
| ATOM | 1612 | CB | ASP | A | 221 | 39.344 | 38.886 | 46.102 | 1.00 | 50.93 | C |
| ATOM | 1613 | CG | ASP | A | 221 | 39.815 | 37.884 | 45.055 | 1.00 | 51.86 | C |
| ATOM | 1614 | OD1 | ASP | A | 221 | 40.826 | 37.194 | 45.294 | 1.00 | 51.97 | O |
| ATOM | 1615 | OD2 | ASP | A | 221 | 39.179 | 37.801 | 43.984 | 1.00 | 52.79 | O |
| ATOM | 1616 | C | ASP | A | 221 | 39.521 | 39.701 | 48.447 | 1.00 | 50.50 | C |
| ATOM | 1617 | O | ASP | A | 221 | 38.752 | 39.620 | 49.405 | 1.00 | 50.41 | O |
| ATOM | 1618 | N | LEU | A | 222 | 40.175 | 40.818 | 48.137 | 1.00 | 49.45 | N |
| ATOM | 1619 | CA | LEU | A | 222 | 40.032 | 42.025 | 48.929 | 1.00 | 48.89 | C |
| ATOM | 1620 | CB | LEU | A | 222 | 41.358 | 42.411 | 49.601 | 1.00 | 47.67 | C |
| ATOM | 1621 | CG | LEU | A | 222 | 41.360 | 43.812 | 50.247 | 1.00 | 46.60 | C |
| ATOM | 1622 | CD1 | LEU | A | 222 | 40.408 | 43.819 | 51.431 | 1.00 | 45.23 | C |
| ATOM | 1623 | CD2 | LEU | A | 222 | 42.755 | 44.207 | 50.704 | 1.00 | 46.47 | C |
| ATOM | 1624 | C | LEU | A | 222 | 39.574 | 43.205 | 48.092 | 1.00 | 49.00 | C |
| ATOM | 1625 | O | LEU | A | 222 | 40.015 | 43.388 | 46.954 | 1.00 | 49.64 | O |
| ATOM | 1626 | N | GLU | A | 223 | 38.676 | 43.997 | 48.662 | 1.00 | 48.21 | N |
| ATOM | 1627 | CA | GLU | A | 223 | 38.188 | 45.202 | 48.020 | 1.00 | 48.95 | C |
| ATOM | 1628 | CB | GLU | A | 223 | 36.721 | 45.066 | 47.609 | 1.00 | 49.50 | C |
| ATOM | 1629 | CG | GLU | A | 223 | 36.506 | 44.446 | 46.243 | 1.00 | 51.80 | C |
| ATOM | 1630 | CD | GLU | A | 223 | 35.033 | 44.406 | 45.837 | 1.00 | 53.35 | C |
| ATOM | 1631 | OE1 | GLU | A | 223 | 34.344 | 45.445 | 45.968 | 1.00 | 53.24 | O |
| ATOM | 1632 | OE2 | GLU | A | 223 | 34.572 | 43.335 | 45.380 | 1.00 | 54.33 | O |
| ATOM | 1633 | C | GLU | A | 223 | 38.327 | 46.268 | 49.090 | 1.00 | 48.51 | C |
| ATOM | 1634 | O | GLU | A | 223 | 37.996 | 46.023 | 50.248 | 1.00 | 48.49 | O |
| ATOM | 1635 | N | ILE | A | 224 | 38.838 | 47.434 | 48.715 | 1.00 | 47.60 | N |
| ATOM | 1636 | CA | ILE | A | 224 | 38.997 | 48.518 | 49.667 | 1.00 | 48.23 | C |
| ATOM | 1637 | CB | ILE | A | 224 | 40.485 | 48.848 | 49.971 | 1.00 | 48.39 | C |
| ATOM | 1638 | CG2 | ILE | A | 224 | 40.567 | 50.044 | 50.930 | 1.00 | 45.55 | C |
| ATOM | 1639 | CG1 | ILE | A | 224 | 41.190 | 47.639 | 50.578 | 1.00 | 47.81 | C |
| ATOM | 1640 | CD1 | ILE | A | 224 | 42.608 | 47.938 | 51.022 | 1.00 | 49.30 | C |
| ATOM | 1641 | C | ILE | A | 224 | 38.380 | 49.777 | 49.112 | 1.00 | 49.09 | C |
| ATOM | 1642 | O | ILE | A | 224 | 38.547 | 50.094 | 47.939 | 1.00 | 49.19 | O |
| ATOM | 1643 | N | GLN | A | 225 | 37.672 | 50.498 | 49.969 | 1.00 | 50.08 | N |
| ATOM | 1644 | CA | GLN | A | 225 | 37.065 | 51.748 | 49.576 | 1.00 | 50.49 | C |
| ATOM | 1645 | CB | GLN | A | 225 | 35.558 | 51.589 | 49.381 | 1.00 | 52.64 | C |
| ATOM | 1646 | CG | GLN | A | 225 | 34.926 | 52.767 | 48.650 | 1.00 | 58.03 | C |
| ATOM | 1647 | CD | GLN | A | 225 | 33.414 | 52.644 | 48.521 | 1.00 | 61.65 | C |
| ATOM | 1648 | OE1 | GLN | A | 225 | 32.882 | 51.547 | 48.301 | 1.00 | 63.50 | O |
| ATOM | 1649 | NE2 | GLN | A | 225 | 32.713 | 53.773 | 48.640 | 1.00 | 62.19 | N |
| ATOM | 1650 | C | GLN | A | 225 | 37.335 | 52.767 | 50.667 | 1.00 | 49.31 | C |
| ATOM | 1651 | O | GLN | A | 225 | 37.206 | 52.467 | 51.849 | 1.00 | 48.85 | O |
| ATOM | 1652 | N | GLU | A | 226 | 37.716 | 53.970 | 50.261 | 1.00 | 49.02 | N |
| ATOM | 1653 | CA | GLU | A | 226 | 37.986 | 55.037 | 51.202 | 1.00 | 49.37 | C |
| ATOM | 1654 | CB | GLU | A | 226 | 39.371 | 55.639 | 50.948 | 1.00 | 49.89 | C |
| ATOM | 1655 | CG | GLU | A | 226 | 40.510 | 54.760 | 51.421 | 1.00 | 52.38 | C |
| ATOM | 1656 | CD | GLU | A | 226 | 41.835 | 55.487 | 51.477 | 1.00 | 53.73 | C |
| ATOM | 1657 | OE1 | GLU | A | 226 | 41.848 | 56.739 | 51.447 | 1.00 | 53.46 | O |
| ATOM | 1658 | OE2 | GLU | A | 226 | 42.867 | 54.795 | 51.564 | 1.00 | 56.58 | O |
| ATOM | 1659 | C | GLU | A | 226 | 36.924 | 56.124 | 51.092 | 1.00 | 49.24 | C |
| ATOM | 1660 | O | GLU | A | 226 | 36.378 | 56.364 | 50.018 | 1.00 | 48.07 | O |
| ATOM | 1661 | N | ILE | A | 227 | 36.623 | 56.760 | 52.221 | 1.00 | 49.24 | N |
| ATOM | 1662 | CA | ILE | A | 227 | 35.657 | 57.842 | 52.263 | 1.00 | 48.85 | C |
| ATOM | 1663 | CB | ILE | A | 227 | 34.319 | 57.415 | 52.920 | 1.00 | 50.12 | C |
| ATOM | 1664 | CG2 | ILE | A | 227 | 33.318 | 58.569 | 52.852 | 1.00 | 49.91 | C |
| ATOM | 1665 | CG1 | ILE | A | 227 | 33.743 | 56.187 | 52.215 | 1.00 | 50.47 | C |
| ATOM | 1666 | CD1 | ILE | A | 227 | 33.350 | 56.434 | 50.772 | 1.00 | 51.78 | C |
| ATOM | 1667 | C | ILE | A | 227 | 36.269 | 58.951 | 53.100 | 1.00 | 48.73 | C |
| ATOM | 1668 | O | ILE | A | 227 | 36.109 | 58.984 | 54.316 | 1.00 | 48.07 | O |
| ATOM | 1669 | N | ASN | A | 228 | 37.004 | 59.843 | 52.455 | 1.00 | 48.66 | N |
| ATOM | 1670 | CA | ASN | A | 228 | 37.607 | 60.946 | 53.178 | 1.00 | 48.65 | C |
| ATOM | 1671 | CB | ASN | A | 228 | 38.877 | 61.416 | 52.472 | 1.00 | 46.19 | C |
| ATOM | 1672 | CG | ASN | A | 228 | 39.741 | 62.285 | 53.348 | 1.00 | 44.45 | C |
| ATOM | 1673 | OD1 | ASN | A | 228 | 39.241 | 63.129 | 54.093 | 1.00 | 44.21 | O |
| ATOM | 1674 | ND2 | ASN | A | 228 | 41.051 | 62.099 | 53.256 | 1.00 | 43.34 | N |
| ATOM | 1675 | C | ASN | A | 228 | 36.528 | 62.013 | 53.117 | 1.00 | 50.32 | C |
| ATOM | 1676 | O | ASN | A | 228 | 36.316 | 62.633 | 52.078 | 1.00 | 50.62 | O |
| ATOM | 1677 | N | ALA | A | 229 | 35.832 | 62.210 | 54.229 | 1.00 | 51.78 | N |
| ATOM | 1678 | CA | ALA | A | 229 | 34.747 | 63.177 | 54.276 | 1.00 | 53.39 | C |
| ATOM | 1679 | CB | ALA | A | 229 | 34.142 | 63.210 | 55.673 | 1.00 | 53.58 | C |
| ATOM | 1680 | C | ALA | A | 229 | 35.162 | 64.582 | 53.840 | 1.00 | 55.09 | C |
| ATOM | 1681 | O | ALA | A | 229 | 34.707 | 65.066 | 52.804 | 1.00 | 55.48 | O |
| ATOM | 1682 | N | ALA | A | 230 | 36.023 | 65.223 | 54.630 | 1.00 | 56.84 | N |
| ATOM | 1683 | CA | ALA | A | 230 | 36.499 | 66.580 | 54.364 | 1.00 | 58.29 | C |
| ATOM | 1684 | CB | ALA | A | 230 | 37.320 | 67.086 | 55.557 | 1.00 | 57.34 | C |
| ATOM | 1685 | C | ALA | A | 230 | 37.302 | 66.757 | 53.073 | 1.00 | 59.59 | C |
| ATOM | 1686 | O | ALA | A | 230 | 37.190 | 67.790 | 52.415 | 1.00 | 60.23 | O |
| ATOM | 1687 | N | ASN | A | 231 | 38.111 | 65.765 | 52.711 | 1.00 | 60.75 | N |
| ATOM | 1688 | CA | ASN | A | 231 | 38.920 | 65.865 | 51.503 | 1.00 | 61.67 | C |
| ATOM | 1689 | CB | ASN | A | 231 | 40.382 | 66.056 | 51.882 | 1.00 | 61.91 | C |
| ATOM | 1690 | CG | ASN | A | 231 | 40.586 | 67.284 | 52.741 | 1.00 | 62.84 | C |
| ATOM | 1691 | OD1 | ASN | A | 231 | 40.150 | 67.327 | 53.896 | 1.00 | 62.73 | O |
| ATOM | 1692 | ND2 | ASN | A | 231 | 41.226 | 68.304 | 52.176 | 1.00 | 63.87 | N |
| ATOM | 1693 | C | ASN | A | 231 | 38.742 | 64.660 | 50.597 | 1.00 | 62.80 | C |
| ATOM | 1694 | O | ASN | A | 231 | 39.599 | 63.790 | 50.484 | 1.00 | 62.64 | O |
| ATOM | 1695 | N | SER | A | 232 | 37.595 | 64.662 | 49.936 | 1.00 | 64.42 | N |
| ATOM | 1696 | CA | SER | A | 232 | 37.142 | 63.621 | 49.017 | 1.00 | 66.00 | C |
| ATOM | 1697 | CB | SER | A | 232 | 36.270 | 64.247 | 47.921 | 1.00 | 65.58 | C |
| ATOM | 1698 | OG | SER | A | 232 | 35.654 | 63.273 | 47.118 | 1.00 | 64.34 | O |
| ATOM | 1699 | C | SER | A | 232 | 38.133 | 62.660 | 48.356 | 1.00 | 67.30 | C |
| ATOM | 1700 | O | SER | A | 232 | 37.844 | 61.478 | 48.241 | 1.00 | 68.34 | O |
| ATOM | 1701 | N | LYS | A | 233 | 39.286 | 63.123 | 47.912 | 1.00 | 68.58 | N |
| ATOM | 1702 | CA | LYS | A | 233 | 40.190 | 62.197 | 47.261 | 1.00 | 69.94 | C |
| ATOM | 1703 | CB | LYS | A | 233 | 40.400 | 62.638 | 45.803 | 1.00 | 71.58 | C |
| ATOM | 1704 | CG | LYS | A | 233 | 39.176 | 62.479 | 44.974 | 1.00 | 73.75 | C |
| ATOM | 1705 | CD | LYS | A | 233 | 39.427 | 62.892 | 43.550 | 1.00 | 75.60 | C |
| ATOM | 1706 | CE | LYS | A | 233 | 38.191 | 62.670 | 42.696 | 1.00 | 75.86 | C |
| ATOM | 1707 | NZ | LYS | A | 233 | 38.459 | 62.985 | 41.265 | 1.00 | 76.61 | N |
| ATOM | 1708 | C | LYS | A | 233 | 41.509 | 61.944 | 47.886 | 1.00 | 69.17 | C |
| ATOM | 1709 | O | LYS | A | 233 | 42.227 | 61.032 | 47.508 | 1.00 | 69.35 | O |
| ATOM | 1710 | N | GLN | A | 234 | 41.851 | 62.763 | 48.842 | 1.00 | 67.96 | N |
| ATOM | 1711 | CA | GLN | A | 234 | 43.142 | 62.551 | 49.466 | 1.00 | 66.83 | C |
| ATOM | 1712 | CB | GLN | A | 234 | 43.403 | 63.627 | 50.528 | 1.00 | 68.97 | C |
| ATOM | 1713 | CG | GLN | A | 234 | 43.559 | 64.992 | 49.966 | 1.00 | 72.57 | C |
| ATOM | 1714 | CD | GLN | A | 234 | 43.873 | 65.967 | 51.073 | 1.00 | 75.05 | C |
| ATOM | 1715 | OE1 | GLN | A | 234 | 44.057 | 67.154 | 50.835 | 1.00 | 76.57 | O |
| ATOM | 1716 | NE2 | GLN | A | 234 | 43.939 | 65.457 | 52.315 | 1.00 | 75.94 | N |
| ATOM | 1717 | C | GLN | A | 234 | 43.182 | 61.204 | 50.174 | 1.00 | 64.24 | C |
| ATOM | 1718 | O | GLN | A | 234 | 42.202 | 60.756 | 50.754 | 1.00 | 63.20 | O |
| ATOM | 1719 | N | PRO | A | 235 | 44.313 | 60.523 | 50.054 | 1.00 | 62.23 | N |
| ATOM | 1720 | CD | PRO | A | 235 | 45.458 | 60.745 | 49.148 | 1.00 | 62.03 | C |
| ATOM | 1721 | CA | PRO | A | 235 | 44.439 | 59.249 | 50.740 | 1.00 | 59.89 | C |
| ATOM | 1722 | CB | PRO | A | 235 | 45.843 | 58.763 | 50.351 | 1.00 | 60.54 | C |
| ATOM | 1723 | CG | PRO | A | 235 | 46.002 | 59.339 | 48.966 | 1.00 | 61.10 | C |
| ATOM | 1724 | C | PRO | A | 235 | 44.291 | 59.465 | 52.236 | 1.00 | 57.74 | C |
| ATOM | 1725 | O | PRO | A | 235 | 44.637 | 60.534 | 52.742 | 1.00 | 56.92 | O |
| ATOM | 1726 | N | ILE | A | 236 | 43.761 | 58.468 | 52.939 | 1.00 | 54.66 | N |
| ATOM | 1727 | CA | ILE | A | 236 | 43.560 | 58.587 | 54.375 | 1.00 | 51.85 | C |
| ATOM | 1728 | CB | ILE | A | 236 | 42.378 | 57.706 | 54.817 | 1.00 | 51.57 | C |
| ATOM | 1729 | CG2 | ILE | A | 236 | 42.255 | 57.690 | 56.343 | 1.00 | 49.53 | C |
| ATOM | 1730 | CG1 | ILE | A | 236 | 41.101 | 58.230 | 54.160 | 1.00 | 49.90 | C |
| ATOM | 1731 | CD1 | ILE | A | 236 | 39.884 | 57.360 | 54.374 | 1.00 | 49.65 | C |
| ATOM | 1732 | C | ILE | A | 236 | 44.805 | 58.217 | 55.174 | 1.00 | 50.45 | C |
| ATOM | 1733 | O | ILE | A | 236 | 45.302 | 57.087 | 55.106 | 1.00 | 49.58 | O |
| ATOM | 1734 | N | HIS | A | 237 | 45.293 | 59.188 | 55.934 | 1.00 | 49.43 | N |
| ATOM | 1735 | CA | HIS | A | 237 | 46.473 | 59.026 | 56.769 | 1.00 | 49.30 | C |
| ATOM | 1736 | CB | HIS | A | 237 | 47.578 | 59.985 | 56.319 | 1.00 | 52.49 | C |
| ATOM | 1737 | CG | HIS | A | 237 | 48.178 | 59.640 | 54.993 | 1.00 | 56.44 | C |
| ATOM | 1738 | CD2 | HIS | A | 237 | 47.823 | 59.990 | 53.733 | 1.00 | 58.66 | C |
| ATOM | 1739 | ND1 | HIS | A | 237 | 49.288 | 58.831 | 54.866 | 1.00 | 58.34 | N |
| ATOM | 1740 | CE1 | HIS | A | 237 | 49.592 | 58.699 | 53.586 | 1.00 | 58.47 | C |
| ATOM | 1741 | NE2 | HIS | A | 237 | 48.719 | 59.392 | 52.877 | 1.00 | 58.99 | N |
| ATOM | 1742 | C | HIS | A | 237 | 46.084 | 59.369 | 58.190 | 1.00 | 46.72 | C |
| ATOM | 1743 | O | HIS | A | 237 | 45.089 | 60.042 | 58.425 | 1.00 | 46.57 | O |
| ATOM | 1744 | N | MET | A | 238 | 46.876 | 58.909 | 59.143 | 1.00 | 44.39 | N |
| ATOM | 1745 | CA | MET | A | 238 | 46.615 | 59.210 | 60.542 | 1.00 | 41.94 | C |
| ATOM | 1746 | CB | MET | A | 238 | 45.530 | 58.285 | 61.118 | 1.00 | 41.23 | C |
| ATOM | 1747 | CG | MET | A | 238 | 45.977 | 56.832 | 61.363 | 1.00 | 40.02 | C |
| ATOM | 1748 | SD | MET | A | 238 | 44.696 | 55.811 | 62.166 | 1.00 | 41.70 | S |
| ATOM | 1749 | CE | MET | A | 238 | 44.354 | 56.771 | 63.642 | 1.00 | 37.46 | C |
| ATOM | 1750 | C | MET | A | 238 | 47.904 | 59.001 | 61.319 | 1.00 | 41.00 | C |
| ATOM | 1751 | O | MET | A | 238 | 48.871 | 58.419 | 60.810 | 1.00 | 40.98 | O |
| ATOM | 1752 | N | VAL | A | 239 | 47.912 | 59.501 | 62.544 | 1.00 | 39.69 | N |
| ATOM | 1753 | CA | VAL | A | 239 | 49.031 | 59.328 | 63.441 | 1.00 | 38.85 | C |
| ATOM | 1754 | CB | VAL | A | 239 | 49.186 | 60.539 | 64.381 | 1.00 | 38.92 | C |
| ATOM | 1755 | CG1 | VAL | A | 239 | 50.327 | 60.292 | 65.371 | 1.00 | 36.40 | C |
| ATOM | 1756 | CG2 | VAL | A | 239 | 49.422 | 61.807 | 63.572 | 1.00 | 38.55 | C |
| ATOM | 1757 | C | VAL | A | 239 | 48.602 | 58.110 | 64.269 | 1.00 | 39.35 | C |
| ATOM | 1758 | O | VAL | A | 239 | 47.556 | 58.146 | 64.933 | 1.00 | 38.62 | O |
| ATOM | 1759 | N | TYR | A | 240 | 49.380 | 57.032 | 64.223 | 1.00 | 38.67 | N |
| ATOM | 1760 | CA | TYR | A | 240 | 49.019 | 55.841 | 64.983 | 1.00 | 38.84 | C |
| ATOM | 1761 | CB | TYR | A | 240 | 47.830 | 55.131 | 64.301 | 1.00 | 37.73 | C |
| ATOM | 1762 | CG | TYR | A | 240 | 47.000 | 54.286 | 65.250 | 1.00 | 37.03 | C |
| ATOM | 1763 | CD1 | TYR | A | 240 | 46.995 | 52.882 | 65.161 | 1.00 | 37.33 | C |
| ATOM | 1764 | CE1 | TYR | A | 240 | 46.284 | 52.091 | 66.088 | 1.00 | 35.55 | C |
| ATOM | 1765 | CD2 | TYR | A | 240 | 46.268 | 54.880 | 66.282 | 1.00 | 35.16 | C |
| ATOM | 1766 | CE2 | TYR | A | 240 | 45.556 | 54.101 | 67.209 | 1.00 | 35.73 | C |
| ATOM | 1767 | CZ | TYR | A | 240 | 45.571 | 52.711 | 67.107 | 1.00 | 35.56 | C |
| ATOM | 1768 | OH | TYR | A | 240 | 44.886 | 51.951 | 68.024 | 1.00 | 33.84 | O |
| ATOM | 1769 | C | TYR | A | 240 | 50.190 | 54.875 | 65.134 | 1.00 | 38.92 | C |
| ATOM | 1770 | O | TYR | A | 240 | 51.279 | 55.117 | 64.605 | 1.00 | 37.92 | O |
| ATOM | 1771 | N | VAL | A | 241 | 49.963 | 53.799 | 65.892 | 1.00 | 39.16 | N |
| ATOM | 1772 | CA | VAL | A | 241 | 50.974 | 52.760 | 66.096 | 1.00 | 38.86 | C |
| ATOM | 1773 | CB | VAL | A | 241 | 50.919 | 52.199 | 67.531 | 1.00 | 38.84 | C |
| ATOM | 1774 | CG1 | VAL | A | 241 | 52.077 | 51.217 | 67.755 | 1.00 | 38.86 | C |
| ATOM | 1775 | CG2 | VAL | A | 241 | 50.997 | 53.350 | 68.548 | 1.00 | 38.26 | C |
| ATOM | 1776 | C | VAL | A | 241 | 50.599 | 51.667 | 65.091 | 1.00 | 39.24 | C |
| ATOM | 1777 | O | VAL | A | 241 | 49.757 | 50.814 | 65.373 | 1.00 | 38.79 | O |
| ATOM | 1778 | N | PRO | A | 242 | 51.218 | 51.688 | 63.897 | 1.00 | 38.54 | N |
| ATOM | 1779 | CD | PRO | A | 242 | 52.282 | 52.598 | 63.432 | 1.00 | 38.32 | C |
| ATOM | 1780 | CA | PRO | A | 242 | 50.903 | 50.688 | 62.871 | 1.00 | 38.58 | C |
| ATOM | 1781 | CB | PRO | A | 242 | 51.844 | 51.055 | 61.710 | 1.00 | 39.00 | C |
| ATOM | 1782 | CG | PRO | A | 242 | 52.972 | 51.768 | 62.377 | 1.00 | 38.88 | C |
| ATOM | 1783 | C | PRO | A | 242 | 50.957 | 49.212 | 63.237 | 1.00 | 38.80 | C |
| ATOM | 1784 | O | PRO | A | 242 | 50.156 | 48.428 | 62.726 | 1.00 | 38.31 | O |
| ATOM | 1785 | N | SER | A | 243 | 51.883 | 48.815 | 64.102 | 1.00 | 38.41 | N |
| ATOM | 1786 | CA | SER | A | 243 | 51.938 | 47.405 | 64.472 | 1.00 | 37.91 | C |
| ATOM | 1787 | CB | SER | A | 243 | 53.125 | 47.123 | 65.405 | 1.00 | 37.61 | C |
| ATOM | 1788 | OG | SER | A | 243 | 52.943 | 47.755 | 66.655 | 1.00 | 41.28 | O |
| ATOM | 1789 | C | SER | A | 243 | 50.612 | 47.040 | 65.155 | 1.00 | 37.17 | C |
| ATOM | 1790 | O | SER | A | 243 | 50.091 | 45.942 | 64.954 | 1.00 | 36.61 | O |
| ATOM | 1791 | N | HIS | A | 244 | 50.069 | 47.968 | 65.944 | 1.00 | 37.26 | N |
| ATOM | 1792 | CA | HIS | A | 244 | 48.794 | 47.742 | 66.627 | 1.00 | 38.09 | C |
| ATOM | 1793 | CB | HIS | A | 244 | 48.445 | 48.911 | 67.552 | 1.00 | 38.96 | C |
| ATOM | 1794 | CG | HIS | A | 244 | 49.245 | 48.968 | 68.812 | 1.00 | 41.77 | C |
| ATOM | 1795 | CD2 | HIS | A | 244 | 50.437 | 48.421 | 69.150 | 1.00 | 43.37 | C |
| ATOM | 1796 | ND1 | HIS | A | 244 | 48.841 | 49.707 | 69.906 | 1.00 | 43.61 | N |
| ATOM | 1797 | CE1 | HIS | A | 244 | 49.747 | 49.612 | 70.862 | 1.00 | 42.99 | C |
| ATOM | 1798 | NE2 | HIS | A | 244 | 50.727 | 48.838 | 70.429 | 1.00 | 43.88 | N |
| ATOM | 1799 | C | HIS | A | 244 | 47.641 | 47.586 | 65.626 | 1.00 | 38.04 | C |
| ATOM | 1800 | O | HIS | A | 244 | 46.785 | 46.706 | 65.772 | 1.00 | 37.57 | O |
| ATOM | 1801 | N | LEU | A | 245 | 47.618 | 48.458 | 64.622 | 1.00 | 37.39 | N |
| ATOM | 1802 | CA | LEU | A | 245 | 46.570 | 48.444 | 63.612 | 1.00 | 37.95 | C |
| ATOM | 1803 | CB | LEU | A | 245 | 46.707 | 49.667 | 62.697 | 1.00 | 37.21 | C |
| ATOM | 1804 | CG | LEU | A | 245 | 45.615 | 49.806 | 61.638 | 1.00 | 36.70 | C |
| ATOM | 1805 | CD1 | LEU | A | 245 | 44.253 | 49.763 | 62.302 | 1.00 | 34.82 | C |
| ATOM | 1806 | CD2 | LEU | A | 245 | 45.800 | 51.096 | 60.875 | 1.00 | 34.56 | C |
| ATOM | 1807 | C | LEU | A | 245 | 46.645 | 47.160 | 62.794 | 1.00 | 38.15 | C |
| ATOM | 1808 | O | LEU | A | 245 | 45.629 | 46.536 | 62.481 | 1.00 | 36.75 | O |
| ATOM | 1809 | N | TYR | A | 246 | 47.872 | 46.776 | 62.461 | 1.00 | 39.36 | N |
| ATOM | 1810 | CA | TYR | A | 246 | 48.150 | 45.562 | 61.701 | 1.00 | 39.15 | C |
| ATOM | 1811 | CB | TYR | A | 246 | 49.670 | 45.409 | 61.528 | 1.00 | 40.30 | C |
| ATOM | 1812 | CG | TYR | A | 246 | 50.085 | 44.257 | 60.640 | 1.00 | 41.33 | C |
| ATOM | 1813 | CD1 | TYR | A | 246 | 50.188 | 42.957 | 61.145 | 1.00 | 41.30 | C |
| ATOM | 1814 | CE1 | TYR | A | 246 | 50.523 | 41.893 | 60.316 | 1.00 | 42.24 | C |
| ATOM | 1815 | CD2 | TYR | A | 246 | 50.332 | 44.461 | 59.283 | 1.00 | 41.32 | C |
| ATOM | 1816 | CE2 | TYR | A | 246 | 50.668 | 43.407 | 58.446 | 1.00 | 42.01 | C |
| ATOM | 1817 | CZ | TYR | A | 246 | 50.761 | 42.126 | 58.971 | 1.00 | 42.97 | C |
| ATOM | 1818 | OH | TYR | A | 246 | 51.102 | 41.077 | 58.153 | 1.00 | 45.16 | O |
| ATOM | 1819 | C | TYR | A | 246 | 47.586 | 44.370 | 62.478 | 1.00 | 39.49 | C |
| ATOM | 1820 | O | TYR | A | 246 | 46.832 | 43.558 | 61.946 | 1.00 | 38.52 | O |
| ATOM | 1821 | N | HIS | A | 247 | 47.957 | 44.278 | 63.749 | 1.00 | 39.23 | N |
| ATOM | 1822 | CA | HIS | A | 247 | 47.479 | 43.200 | 64.594 | 1.00 | 39.95 | C |
| ATOM | 1823 | CB | HIS | A | 247 | 47.866 | 43.466 | 66.050 | 1.00 | 41.41 | C |
| ATOM | 1824 | CG | HIS | A | 247 | 47.498 | 42.353 | 66.979 | 1.00 | 44.58 | C |
| ATOM | 1825 | CD2 | HIS | A | 247 | 46.340 | 42.078 | 67.630 | 1.00 | 46.03 | C |
| ATOM | 1826 | ND1 | HIS | A | 247 | 48.368 | 41.335 | 67.300 | 1.00 | 46.97 | N |
| ATOM | 1827 | CE1 | HIS | A | 247 | 47.764 | 40.481 | 68.110 | 1.00 | 47.08 | C |
| ATOM | 1828 | NE2 | HIS | A | 247 | 46.533 | 40.909 | 68.326 | 1.00 | 46.17 | N |
| ATOM | 1829 | C | HIS | A | 247 | 45.952 | 43.050 | 64.486 | 1.00 | 38.96 | C |
| ATOM | 1830 | O | HIS | A | 247 | 45.446 | 41.966 | 64.166 | 1.00 | 38.68 | O |
| ATOM | 1831 | N | MET | A | 248 | 45.228 | 44.137 | 64.745 | 1.00 | 37.25 | N |
| ATOM | 1832 | CA | MET | A | 248 | 43.766 | 44.110 | 64.691 | 1.00 | 37.09 | C |
| ATOM | 1833 | CB | MET | A | 248 | 43.170 | 45.482 | 65.062 | 1.00 | 35.49 | C |
| ATOM | 1834 | CG | MET | A | 248 | 43.369 | 45.864 | 66.521 | 1.00 | 35.15 | C |
| ATOM | 1835 | SD | MET | A | 248 | 42.761 | 47.509 | 66.954 | 1.00 | 35.90 | S |
| ATOM | 1836 | CE | MET | A | 248 | 43.949 | 48.517 | 66.081 | 1.00 | 35.31 | C |
| ATOM | 1837 | C | MET | A | 248 | 43.237 | 43.664 | 63.334 | 1.00 | 36.22 | C |
| ATOM | 1838 | O | MET | A | 248 | 42.370 | 42.790 | 63.263 | 1.00 | 35.68 | O |
| ATOM | 1839 | N | LEU | A | 249 | 43.768 | 44.250 | 62.263 | 1.00 | 35.96 | N |
| ATOM | 1840 | CA | LEU | A | 249 | 43.334 | 43.909 | 60.912 | 1.00 | 35.57 | C |
| ATOM | 1841 | CB | LEU | A | 249 | 43.961 | 44.871 | 59.904 | 1.00 | 34.49 | C |
| ATOM | 1842 | CG | LEU | A | 249 | 43.323 | 46.265 | 59.901 | 1.00 | 34.55 | C |
| ATOM | 1843 | CD1 | LEU | A | 249 | 44.151 | 47.220 | 59.023 | 1.00 | 35.09 | C |
| ATOM | 1844 | CD2 | LEU | A | 249 | 41.888 | 46.163 | 59.392 | 1.00 | 32.39 | C |
| ATOM | 1845 | C | LEU | A | 249 | 43.660 | 42.472 | 60.551 | 1.00 | 35.35 | C |
| ATOM | 1846 | O | LEU | A | 249 | 42.833 | 41.766 | 59.973 | 1.00 | 36.69 | O |
| ATOM | 1847 | N | PHE | A | 250 | 44.861 | 42.038 | 60.903 | 1.00 | 35.77 | N |
| ATOM | 1848 | CA | PHE | A | 250 | 45.291 | 40.677 | 60.629 | 1.00 | 36.72 | C |
| ATOM | 1849 | CB | PHE | A | 250 | 46.751 | 40.484 | 61.096 | 1.00 | 36.49 | C |
| ATOM | 1850 | CG | PHE | A | 250 | 47.261 | 39.081 | 60.923 | 1.00 | 36.37 | C |
| ATOM | 1851 | CD1 | PHE | A | 250 | 47.279 | 38.492 | 59.668 | 1.00 | 35.65 | C |
| ATOM | 1852 | CD2 | PHE | A | 250 | 47.691 | 38.334 | 62.021 | 1.00 | 38.12 | C |
| ATOM | 1853 | CE1 | PHE | A | 250 | 47.713 | 37.178 | 59.502 | 1.00 | 34.80 | C |
| ATOM | 1854 | CE2 | PHE | A | 250 | 48.132 | 37.007 | 61.863 | 1.00 | 36.08 | C |
| ATOM | 1855 | CZ | PHE | A | 250 | 48.137 | 36.439 | 60.598 | 1.00 | 35.31 | C |
| ATOM | 1856 | C | PHE | A | 250 | 44.372 | 39.646 | 61.311 | 1.00 | 36.30 | C |
| ATOM | 1857 | O | PHE | A | 250 | 43.951 | 38.659 | 60.691 | 1.00 | 36.09 | O |
| ATOM | 1858 | N | GLU | A | 251 | 44.061 | 39.872 | 62.584 | 1.00 | 36.36 | N |
| ATOM | 1859 | CA | GLU | A | 251 | 43.190 | 38.954 | 63.313 | 1.00 | 37.22 | C |
| ATOM | 1860 | CB | GLU | A | 251 | 43.136 | 39.310 | 64.810 | 1.00 | 38.83 | C |
| ATOM | 1861 | CG | GLU | A | 251 | 44.437 | 39.070 | 65.599 | 1.00 | 41.99 | C |
| ATOM | 1862 | CD | GLU | A | 251 | 44.900 | 37.609 | 65.584 | 1.00 | 45.12 | C |
| ATOM | 1863 | OE1 | GLU | A | 251 | 44.072 | 36.707 | 65.859 | 1.00 | 44.37 | O |
| ATOM | 1864 | OE2 | GLU | A | 251 | 46.100 | 37.367 | 65.308 | 1.00 | 46.98 | O |
| ATOM | 1865 | C | GLU | A | 251 | 41.777 | 38.960 | 62.728 | 1.00 | 36.71 | C |
| ATOM | 1866 | O | GLU | A | 251 | 41.142 | 37.908 | 62.603 | 1.00 | 36.62 | O |
| ATOM | 1867 | N | LEU | A | 252 | 41.285 | 40.137 | 62.354 | 1.00 | 35.66 | N |
| ATOM | 1868 | CA | LEU | A | 252 | 39.949 | 40.209 | 61.786 | 1.00 | 34.64 | C |
| ATOM | 1869 | CB | LEU | A | 252 | 39.469 | 41.662 | 61.701 | 1.00 | 34.37 | C |
| ATOM | 1870 | CG | LEU | A | 252 | 39.203 | 42.291 | 63.069 | 1.00 | 33.85 | C |
| ATOM | 1871 | CD1 | LEU | A | 252 | 38.665 | 43.688 | 62.910 | 1.00 | 32.68 | C |
| ATOM | 1872 | CD2 | LEU | A | 252 | 38.204 | 41.446 | 63.828 | 1.00 | 32.86 | C |
| ATOM | 1873 | C | LEU | A | 252 | 39.930 | 39.550 | 60.411 | 1.00 | 34.40 | C |
| ATOM | 1874 | O | LEU | A | 252 | 38.949 | 38.901 | 60.044 | 1.00 | 33.09 | O |
| ATOM | 1875 | N | PHE | A | 253 | 41.016 | 39.699 | 59.650 | 1.00 | 34.79 | N |
| ATOM | 1876 | CA | PHE | A | 253 | 41.072 | 39.064 | 58.335 | 1.00 | 35.13 | C |
| ATOM | 1877 | CB | PHE | A | 253 | 42.307 | 39.525 | 57.547 | 1.00 | 33.32 | C |
| ATOM | 1878 | CG | PHE | A | 253 | 42.080 | 40.768 | 56.724 | 1.00 | 30.96 | C |
| ATOM | 1879 | CD1 | PHE | A | 253 | 41.080 | 40.801 | 55.751 | 1.00 | 29.73 | C |
| ATOM | 1880 | CD2 | PHE | A | 253 | 42.893 | 41.887 | 56.891 | 1.00 | 29.16 | C |
| ATOM | 1881 | CE1 | PHE | A | 253 | 40.897 | 41.934 | 54.945 | 1.00 | 29.00 | C |
| ATOM | 1882 | CE2 | PHE | A | 253 | 42.722 | 43.031 | 56.096 | 1.00 | 26.92 | C |
| ATOM | 1883 | CZ | PHE | A | 253 | 41.723 | 43.056 | 55.118 | 1.00 | 29.03 | C |
| ATOM | 1884 | C | PHE | A | 253 | 41.058 | 37.531 | 58.443 | 1.00 | 35.15 | C |
| ATOM | 1885 | O | PHE | A | 253 | 40.404 | 36.853 | 57.644 | 1.00 | 35.44 | O |
| ATOM | 1886 | N | LYS | A | 254 | 41.764 | 36.988 | 59.431 | 1.00 | 36.70 | N |
| ATOM | 1887 | CA | LYS | A | 254 | 41.803 | 35.537 | 59.621 | 1.00 | 36.86 | C |
| ATOM | 1888 | CB | LYS | A | 254 | 42.738 | 35.178 | 60.780 | 1.00 | 37.43 | C |
| ATOM | 1889 | CG | LYS | A | 254 | 44.219 | 35.224 | 60.401 | 1.00 | 39.02 | C |
| ATOM | 1890 | CD | LYS | A | 254 | 45.136 | 35.491 | 61.601 | 1.00 | 39.71 | C |
| ATOM | 1891 | CE | LYS | A | 254 | 45.024 | 34.462 | 62.700 | 1.00 | 39.31 | C |
| ATOM | 1892 | NZ | LYS | A | 254 | 45.811 | 34.906 | 63.889 | 1.00 | 38.90 | N |
| ATOM | 1893 | C | LYS | A | 254 | 40.414 | 34.977 | 59.882 | 1.00 | 37.80 | C |
| ATOM | 1894 | O | LYS | A | 254 | 40.045 | 33.923 | 59.349 | 1.00 | 37.03 | O |
| ATOM | 1895 | N | ASN | A | 255 | 39.647 | 35.692 | 60.705 | 1.00 | 37.78 | N |
| ATOM | 1896 | CA | ASN | A | 255 | 38.288 | 35.283 | 61.043 | 1.00 | 36.87 | C |
| ATOM | 1897 | CB | ASN | A | 255 | 37.697 | 36.203 | 62.112 | 1.00 | 36.84 | C |
| ATOM | 1898 | CG | ASN | A | 255 | 38.407 | 36.067 | 63.444 | 1.00 | 37.83 | C |
| ATOM | 1899 | OD1 | ASN | A | 255 | 39.091 | 35.067 | 63.683 | 1.00 | 36.91 | O |
| ATOM | 1900 | ND2 | ASN | A | 255 | 38.246 | 37.063 | 64.324 | 1.00 | 33.94 | N |
| ATOM | 1901 | C | ASN | A | 255 | 37.404 | 35.298 | 59.806 | 1.00 | 37.58 | C |
| ATOM | 1902 | O | ASN | A | 255 | 36.797 | 34.289 | 59.455 | 1.00 | 37.53 | O |
| ATOM | 1903 | N | ALA | A | 256 | 37.349 | 36.445 | 59.144 | 1.00 | 38.14 | N |
| ATOM | 1904 | CA | ALA | A | 256 | 36.543 | 36.597 | 57.947 | 1.00 | 40.00 | C |
| ATOM | 1905 | CB | ALA | A | 256 | 36.708 | 38.024 | 57.381 | 1.00 | 38.93 | C |
| ATOM | 1906 | C | ALA | A | 256 | 36.899 | 35.547 | 56.883 | 1.00 | 41.37 | C |
| ATOM | 1907 | O | ALA | A | 256 | 36.009 | 34.985 | 56.241 | 1.00 | 42.61 | O |
| ATOM | 1908 | N | MET | A | 257 | 38.190 | 35.282 | 56.693 | 1.00 | 41.93 | N |
| ATOM | 1909 | CA | MET | A | 257 | 38.612 | 34.288 | 55.707 | 1.00 | 43.38 | C |
| ATOM | 1910 | CB | MET | A | 257 | 40.140 | 34.281 | 55.577 | 1.00 | 42.96 | C |
| ATOM | 1911 | CG | MET | A | 257 | 40.651 | 35.362 | 54.652 | 1.00 | 43.17 | C |
| ATOM | 1912 | SD | MET | A | 257 | 42.426 | 35.666 | 54.760 | 1.00 | 45.62 | S |
| ATOM | 1913 | CE | MET | A | 257 | 42.540 | 37.091 | 53.681 | 1.00 | 39.13 | C |
| ATOM | 1914 | C | MET | A | 257 | 38.112 | 32.893 | 56.065 | 1.00 | 43.65 | C |
| ATOM | 1915 | O | MET | A | 257 | 37.571 | 32.188 | 55.226 | 1.00 | 43.23 | O |
| ATOM | 1916 | N | ARG | A | 258 | 38.283 | 32.514 | 57.324 | 1.00 | 45.11 | N |
| ATOM | 1917 | CA | ARG | A | 258 | 37.854 | 31.211 | 57.798 | 1.00 | 46.75 | C |
| ATOM | 1918 | CB | ARG | A | 258 | 38.130 | 31.101 | 59.293 | 1.00 | 48.72 | C |
| ATOM | 1919 | CG | ARG | A | 258 | 38.486 | 29.708 | 59.733 | 1.00 | 52.04 | C |
| ATOM | 1920 | CD | ARG | A | 258 | 37.445 | 29.084 | 60.628 | 1.00 | 53.99 | C |
| ATOM | 1921 | NE | ARG | A | 258 | 37.697 | 29.348 | 62.042 | 1.00 | 57.79 | N |
| ATOM | 1922 | CZ | ARG | A | 258 | 37.036 | 28.762 | 63.040 | 1.00 | 59.30 | C |
| ATOM | 1923 | NH1 | ARG | A | 258 | 36.085 | 27.873 | 62.782 | 1.00 | 59.87 | N |
| ATOM | 1924 | NH2 | ARG | A | 258 | 37.316 | 29.078 | 64.299 | 1.00 | 59.44 | N |
| ATOM | 1925 | C | ARG | A | 258 | 36.364 | 31.023 | 57.544 | 1.00 | 47.62 | C |
| ATOM | 1926 | O | ARG | A | 258 | 35.938 | 30.060 | 56.906 | 1.00 | 47.07 | O |
| ATOM | 1927 | N | ALA | A | 259 | 35.580 | 31.969 | 58.051 | 1.00 | 46.84 | N |
| ATOM | 1928 | CA | ALA | A | 259 | 34.135 | 31.923 | 57.921 | 1.00 | 46.54 | C |
| ATOM | 1929 | CB | ALA | A | 259 | 33.499 | 33.111 | 58.676 | 1.00 | 44.83 | C |
| ATOM | 1930 | C | ALA | A | 259 | 33.675 | 31.908 | 56.468 | 1.00 | 45.67 | C |
| ATOM | 1931 | O | ALA | A | 259 | 32.744 | 31.185 | 56.123 | 1.00 | 46.23 | O |
| ATOM | 1932 | N | THR | A | 260 | 34.318 | 32.700 | 55.619 | 1.00 | 45.57 | N |
| ATOM | 1933 | CA | THR | A | 260 | 33.923 | 32.756 | 54.220 | 1.00 | 47.43 | C |
| ATOM | 1934 | CB | THR | A | 260 | 34.642 | 33.884 | 53.468 | 1.00 | 47.19 | C |
| ATOM | 1935 | OG1 | THR | A | 260 | 34.368 | 35.137 | 54.107 | 1.00 | 46.19 | O |
| ATOM | 1936 | CG2 | THR | A | 260 | 34.161 | 33.938 | 52.014 | 1.00 | 46.97 | C |
| ATOM | 1937 | C | THR | A | 260 | 34.214 | 31.452 | 53.499 | 1.00 | 48.18 | C |
| ATOM | 1938 | O | THR | A | 260 | 33.370 | 30.921 | 52.775 | 1.00 | 48.31 | O |
| ATOM | 1939 | N | VAL | A | 261 | 35.417 | 30.938 | 53.712 | 1.00 | 49.44 | N |
| ATOM | 1940 | CA | VAL | A | 261 | 35.850 | 29.703 | 53.086 | 1.00 | 49.97 | C |
| ATOM | 1941 | CB | VAL | A | 261 | 37.347 | 29.449 | 53.367 | 1.00 | 48.80 | C |
| ATOM | 1942 | CG1 | VAL | A | 261 | 37.713 | 28.031 | 52.987 | 1.00 | 49.51 | C |
| ATOM | 1943 | CG2 | VAL | A | 261 | 38.200 | 30.437 | 52.582 | 1.00 | 47.82 | C |
| ATOM | 1944 | C | VAL | A | 261 | 35.048 | 28.499 | 53.564 | 1.00 | 51.09 | C |
| ATOM | 1945 | O | VAL | A | 261 | 34.590 | 27.688 | 52.768 | 1.00 | 51.68 | O |
| ATOM | 1946 | N | GLU | A | 262 | 34.869 | 28.389 | 54.867 | 1.00 | 52.25 | N |
| ATOM | 1947 | CA | GLU | A | 262 | 34.156 | 27.256 | 55.409 | 1.00 | 54.80 | C |
| ATOM | 1948 | CB | GLU | A | 262 | 34.401 | 27.160 | 56.911 | 1.00 | 54.80 | C |
| ATOM | 1949 | CG | GLU | A | 262 | 35.850 | 26.920 | 57.266 | 1.00 | 55.53 | C |
| ATOM | 1950 | CD | GLU | A | 262 | 36.032 | 26.575 | 58.724 | 1.00 | 57.20 | C |
| ATOM | 1951 | OE1 | GLU | A | 262 | 37.189 | 26.367 | 59.152 | 1.00 | 58.04 | O |
| ATOM | 1952 | OE2 | GLU | A | 262 | 35.017 | 26.505 | 59.449 | 1.00 | 57.62 | O |
| ATOM | 1953 | C | GLU | A | 262 | 32.666 | 27.233 | 55.127 | 1.00 | 56.37 | C |
| ATOM | 1954 | O | GLU | A | 262 | 32.052 | 26.162 | 55.080 | 1.00 | 57.47 | O |
| ATOM | 1955 | N | SER | A | 263 | 32.076 | 28.405 | 54.941 | 1.00 | 57.46 | N |
| ATOM | 1956 | CA | SER | A | 263 | 30.645 | 28.476 | 54.675 | 1.00 | 58.16 | C |
| ATOM | 1957 | CB | SER | A | 263 | 30.066 | 29.778 | 55.239 | 1.00 | 57.77 | C |
| ATOM | 1958 | OG | SER | A | 263 | 30.516 | 30.900 | 54.501 | 1.00 | 57.80 | O |
| ATOM | 1959 | C | SER | A | 263 | 30.359 | 28.388 | 53.181 | 1.00 | 58.64 | C |
| ATOM | 1960 | O | SER | A | 263 | 29.209 | 28.484 | 52.764 | 1.00 | 58.54 | O |
| ATOM | 1961 | N | HIS | A | 264 | 31.405 | 28.213 | 52.377 | 1.00 | 60.09 | N |
| ATOM | 1962 | CA | HIS | A | 264 | 31.239 | 28.112 | 50.928 | 1.00 | 61.82 | C |
| ATOM | 1963 | CB | HIS | A | 264 | 31.712 | 29.381 | 50.232 | 1.00 | 61.09 | C |
| ATOM | 1964 | CG | HIS | A | 264 | 30.823 | 30.557 | 50.455 | 1.00 | 61.36 | C |
| ATOM | 1965 | CD2 | HIS | A | 264 | 29.992 | 31.217 | 49.615 | 1.00 | 61.06 | C |
| ATOM | 1966 | ND1 | HIS | A | 264 | 30.728 | 31.195 | 51.672 | 1.00 | 61.12 | N |
| ATOM | 1967 | CE1 | HIS | A | 264 | 29.878 | 32.201 | 51.571 | 1.00 | 61.35 | C |
| ATOM | 1968 | NE2 | HIS | A | 264 | 29.418 | 32.236 | 50.333 | 1.00 | 60.83 | N |
| ATOM | 1969 | C | HIS | A | 264 | 31.965 | 26.947 | 50.292 | 1.00 | 63.04 | C |
| ATOM | 1970 | O | HIS | A | 264 | 32.336 | 27.019 | 49.123 | 1.00 | 63.58 | O |
| ATOM | 1971 | N | GLU | A | 265 | 32.180 | 25.878 | 51.040 | 1.00 | 64.88 | N |
| ATOM | 1972 | CA | GLU | A | 265 | 32.877 | 24.741 | 50.472 | 1.00 | 67.19 | C |
| ATOM | 1973 | CB | GLU | A | 265 | 33.285 | 23.774 | 51.588 | 1.00 | 67.76 | C |
| ATOM | 1974 | CG | GLU | A | 265 | 34.169 | 24.442 | 52.632 | 1.00 | 69.25 | C |
| ATOM | 1975 | CD | GLU | A | 265 | 34.672 | 23.498 | 53.704 | 1.00 | 70.43 | C |
| ATOM | 1976 | OE1 | GLU | A | 265 | 33.832 | 22.864 | 54.379 | 1.00 | 70.70 | O |
| ATOM | 1977 | OE2 | GLU | A | 265 | 35.910 | 23.401 | 53.875 | 1.00 | 71.10 | O |
| ATOM | 1978 | C | GLU | A | 265 | 31.993 | 24.050 | 49.433 | 1.00 | 67.91 | C |
| ATOM | 1979 | O | GLU | A | 265 | 32.082 | 22.835 | 49.253 | 1.00 | 68.68 | O |
| ATOM | 1980 | N | SER | A | 266 | 31.155 | 24.829 | 48.741 | 1.00 | 68.10 | N |
| ATOM | 1981 | CA | SER | A | 266 | 30.248 | 24.278 | 47.732 | 1.00 | 68.25 | C |
| ATOM | 1982 | CB | SER | A | 266 | 29.142 | 23.460 | 48.397 | 1.00 | 67.50 | C |
| ATOM | 1983 | OG | SER | A | 266 | 29.612 | 22.702 | 49.494 | 1.00 | 67.44 | O |
| ATOM | 1984 | C | SER | A | 266 | 29.548 | 25.319 | 46.880 | 1.00 | 69.01 | C |
| ATOM | 1985 | O | SER | A | 266 | 28.327 | 25.311 | 46.817 | 1.00 | 70.19 | O |
| ATOM | 1986 | N | SER | A | 267 | 30.273 | 26.195 | 46.203 | 1.00 | 69.63 | N |
| ATOM | 1987 | CA | SER | A | 267 | 29.607 | 27.227 | 45.395 | 1.00 | 70.38 | C |
| ATOM | 1988 | CB | SER | A | 267 | 28.565 | 27.955 | 46.251 | 1.00 | 70.35 | C |
| ATOM | 1989 | OG | SER | A | 267 | 28.846 | 27.783 | 47.633 | 1.00 | 69.87 | O |
| ATOM | 1990 | C | SER | A | 267 | 30.759 | 28.133 | 45.111 | 1.00 | 70.71 | C |
| ATOM | 1991 | O | SER | A | 267 | 31.514 | 27.907 | 44.159 | 1.00 | 70.98 | O |
| ATOM | 1992 | N | LEU | A | 268 | 30.836 | 29.196 | 45.908 | 1.00 | 71.00 | N |
| ATOM | 1993 | CA | LEU | A | 268 | 31.989 | 30.053 | 45.880 | 1.00 | 69.79 | C |
| ATOM | 1994 | CB | LEU | A | 268 | 32.921 | 29.601 | 44.764 | 1.00 | 71.11 | C |
| ATOM | 1995 | CG | LEU | A | 268 | 34.216 | 30.382 | 44.686 | 1.00 | 72.28 | C |
| ATOM | 1996 | CD1 | LEU | A | 268 | 35.432 | 29.485 | 44.686 | 1.00 | 72.82 | C |
| ATOM | 1997 | CD2 | LEU | A | 268 | 34.097 | 31.224 | 43.465 | 1.00 | 72.96 | C |
| ATOM | 1998 | C | LEU | A | 268 | 32.174 | 31.541 | 46.031 | 1.00 | 67.90 | C |
| ATOM | 1999 | O | LEU | A | 268 | 31.613 | 32.446 | 45.387 | 1.00 | 68.12 | O |
| ATOM | 2000 | N | ILE | A | 269 | 33.160 | 31.626 | 46.904 | 1.00 | 65.14 | N |
| ATOM | 2001 | CA | ILE | A | 269 | 33.905 | 32.684 | 47.510 | 1.00 | 62.30 | C |
| ATOM | 2002 | CB | ILE | A | 269 | 35.372 | 32.205 | 47.423 | 1.00 | 62.14 | C |
| ATOM | 2003 | CG2 | ILE | A | 269 | 36.042 | 32.724 | 46.167 | 1.00 | 64.34 | C |
| ATOM | 2004 | CG1 | ILE | A | 269 | 36.095 | 32.492 | 48.722 | 1.00 | 62.28 | C |
| ATOM | 2005 | CD1 | ILE | A | 269 | 37.219 | 31.510 | 48.978 | 1.00 | 61.76 | C |
| ATOM | 2006 | C | ILE | A | 269 | 33.761 | 34.171 | 47.231 | 1.00 | 58.98 | C |
| ATOM | 2007 | O | ILE | A | 269 | 34.381 | 34.740 | 46.333 | 1.00 | 59.58 | O |
| ATOM | 2008 | N | PRO | A | 271 | 32.905 | 34.820 | 48.024 | 1.00 | 56.08 | N |
| ATOM | 2009 | CD | PRO | A | 271 | 31.908 | 34.177 | 48.896 | 1.00 | 54.31 | C |
| ATOM | 2010 | CA | PRO | A | 271 | 32.657 | 36.256 | 47.917 | 1.00 | 53.61 | C |
| ATOM | 2011 | CB | PRO | A | 271 | 31.358 | 36.444 | 48.706 | 1.00 | 53.00 | C |
| ATOM | 2012 | CG | PRO | A | 271 | 31.402 | 35.337 | 49.696 | 1.00 | 53.98 | C |
| ATOM | 2013 | C | PRO | A | 271 | 33.865 | 36.953 | 48.564 | 1.00 | 51.22 | C |
| ATOM | 2014 | O | PRO | A | 271 | 34.521 | 36.404 | 49.461 | 1.00 | 49.64 | O |
| ATOM | 2015 | N | PRO | A | 272 | 34.197 | 38.154 | 48.089 | 1.00 | 49.01 | N |
| ATOM | 2016 | CD | PRO | A | 272 | 33.624 | 38.841 | 46.918 | 1.00 | 48.50 | C |
| ATOM | 2017 | CA | PRO | A | 272 | 35.327 | 38.908 | 48.626 | 1.00 | 48.56 | C |
| ATOM | 2018 | CB | PRO | A | 272 | 35.575 | 39.951 | 47.548 | 1.00 | 48.27 | C |
| ATOM | 2019 | CG | PRO | A | 272 | 34.177 | 40.238 | 47.073 | 1.00 | 48.86 | C |
| ATOM | 2020 | C | PRO | A | 272 | 35.069 | 39.562 | 49.971 | 1.00 | 47.43 | C |
| ATOM | 2021 | O | PRO | A | 272 | 33.929 | 39.800 | 50.368 | 1.00 | 47.78 | O |
| ATOM | 2022 | N | ILE | A | 273 | 36.154 | 39.850 | 50.669 | 1.00 | 46.16 | N |
| ATOM | 2023 | CA | ILE | A | 273 | 36.071 | 40.535 | 51.935 | 1.00 | 44.63 | C |
| ATOM | 2024 | CB | ILE | A | 273 | 37.192 | 40.090 | 52.865 | 1.00 | 42.34 | C |
| ATOM | 2025 | CG2 | ILE | A | 273 | 37.263 | 41.002 | 54.073 | 1.00 | 41.76 | C |
| ATOM | 2026 | CG1 | ILE | A | 273 | 36.948 | 38.637 | 53.271 | 1.00 | 40.67 | C |
| ATOM | 2027 | CD1 | ILE | A | 273 | 37.952 | 38.093 | 54.230 | 1.00 | 41.33 | C |
| ATOM | 2028 | C | ILE | A | 273 | 36.214 | 42.011 | 51.567 | 1.00 | 44.85 | C |
| ATOM | 2029 | O | ILE | A | 273 | 37.194 | 42.400 | 50.937 | 1.00 | 44.66 | O |
| ATOM | 2030 | N | LYS | A | 274 | 35.213 | 42.817 | 51.921 | 1.00 | 44.55 | N |
| ATOM | 2031 | CA | LYS | A | 274 | 35.231 | 44.239 | 51.604 | 1.00 | 44.89 | C |
| ATOM | 2032 | CB | LYS | A | 274 | 33.866 | 44.692 | 51.080 | 1.00 | 45.93 | C |
| ATOM | 2033 | CG | LYS | A | 274 | 33.387 | 43.896 | 49.887 | 1.00 | 48.51 | C |
| ATOM | 2034 | CD | LYS | A | 274 | 32.078 | 44.428 | 49.324 | 1.00 | 50.90 | C |
| ATOM | 2035 | CE | LYS | A | 274 | 31.538 | 43.477 | 48.261 | 1.00 | 53.31 | C |
| ATOM | 2036 | NZ | LYS | A | 274 | 30.507 | 44.112 | 47.391 | 1.00 | 56.12 | N |
| ATOM | 2037 | C | LYS | A | 274 | 35.602 | 45.068 | 52.816 | 1.00 | 43.66 | C |
| ATOM | 2038 | O | LYS | A | 274 | 35.115 | 44.836 | 53.915 | 1.00 | 45.19 | O |
| ATOM | 2039 | N | VAL | A | 275 | 36.478 | 46.036 | 52.615 | 1.00 | 42.16 | N |
| ATOM | 2040 | CA | VAL | A | 275 | 36.893 | 46.879 | 53.708 | 1.00 | 40.93 | C |
| ATOM | 2041 | CB | VAL | A | 275 | 38.399 | 46.725 | 54.054 | 1.00 | 38.66 | C |
| ATOM | 2042 | CG1 | VAL | A | 275 | 38.836 | 47.865 | 54.968 | 1.00 | 37.61 | C |
| ATOM | 2043 | CG2 | VAL | A | 275 | 38.649 | 45.400 | 54.745 | 1.00 | 38.50 | C |
| ATOM | 2044 | C | VAL | A | 275 | 36.659 | 48.313 | 53.333 | 1.00 | 40.94 | C |
| ATOM | 2045 | O | VAL | A | 275 | 36.982 | 48.738 | 52.224 | 1.00 | 41.43 | O |
| ATOM | 2046 | N | MET | A | 276 | 36.085 | 49.061 | 54.259 | 1.00 | 41.12 | N |
| ATOM | 2047 | CA | MET | A | 276 | 35.877 | 50.456 | 54.014 | 1.00 | 41.62 | C |
| ATOM | 2048 | CB | MET | A | 276 | 34.407 | 50.810 | 54.006 | 1.00 | 44.65 | C |
| ATOM | 2049 | CG | MET | A | 276 | 34.203 | 52.188 | 53.408 | 1.00 | 50.82 | C |
| ATOM | 2050 | SD | MET | A | 276 | 32.526 | 52.783 | 53.556 | 1.00 | 57.91 | S |
| ATOM | 2051 | CE | MET | A | 276 | 32.709 | 53.783 | 54.936 | 1.00 | 54.36 | C |
| ATOM | 2052 | C | MET | A | 276 | 36.576 | 51.268 | 55.083 | 1.00 | 39.45 | C |
| ATOM | 2053 | O | MET | A | 276 | 36.506 | 50.955 | 56.271 | 1.00 | 38.98 | O |
| ATOM | 2054 | N | VAL | A | 277 | 37.264 | 52.312 | 54.646 | 1.00 | 37.82 | N |
| ATOM | 2055 | CA | VAL | A | 277 | 37.960 | 53.192 | 55.557 | 1.00 | 35.68 | C |
| ATOM | 2056 | CB | VAL | A | 277 | 39.477 | 53.295 | 55.226 | 1.00 | 34.74 | C |
| ATOM | 2057 | CG1 | VAL | A | 277 | 40.164 | 54.210 | 56.241 | 1.00 | 33.86 | C |
| ATOM | 2058 | CG2 | VAL | A | 277 | 40.117 | 51.916 | 55.266 | 1.00 | 32.41 | C |
| ATOM | 2059 | C | VAL | A | 277 | 37.299 | 54.545 | 55.411 | 1.00 | 35.32 | C |
| ATOM | 2060 | O | VAL | A | 277 | 37.304 | 55.145 | 54.332 | 1.00 | 35.25 | O |
| ATOM | 2061 | N | ALA | A | 278 | 36.709 | 55.005 | 56.506 | 1.00 | 35.59 | N |
| ATOM | 2062 | CA | ALA | A | 278 | 36.012 | 56.280 | 56.535 | 1.00 | 35.12 | C |
| ATOM | 2063 | CB | ALA | A | 278 | 34.564 | 56.066 | 56.934 | 1.00 | 32.10 | C |
| ATOM | 2064 | C | ALA | A | 278 | 36.689 | 57.212 | 57.517 | 1.00 | 35.37 | C |
| ATOM | 2065 | O | ALA | A | 278 | 36.995 | 56.814 | 58.629 | 1.00 | 35.85 | O |
| ATOM | 2066 | N | LEU | A | 279 | 36.928 | 58.448 | 57.090 | 1.00 | 36.49 | N |
| ATOM | 2067 | CA | LEU | A | 279 | 37.542 | 59.454 | 57.939 | 1.00 | 36.77 | C |
| ATOM | 2068 | CB | LEU | A | 279 | 38.787 | 60.043 | 57.277 | 1.00 | 36.76 | C |
| ATOM | 2069 | CG | LEU | A | 279 | 39.363 | 61.248 | 58.019 | 1.00 | 37.09 | C |
| ATOM | 2070 | CD1 | LEU | A | 279 | 39.613 | 60.882 | 59.480 | 1.00 | 37.45 | C |
| ATOM | 2071 | CD2 | LEU | A | 279 | 40.652 | 61.698 | 57.345 | 1.00 | 36.96 | C |
| ATOM | 2072 | C | LEU | A | 279 | 36.535 | 60.558 | 58.198 | 1.00 | 38.18 | C |
| ATOM | 2073 | O | LEU | A | 279 | 36.125 | 61.257 | 57.281 | 1.00 | 39.93 | O |
| ATOM | 2074 | N | GLY | A | 280 | 36.123 | 60.696 | 59.449 | 1.00 | 39.13 | N |
| ATOM | 2075 | CA | GLY | A | 280 | 35.167 | 61.722 | 59.812 | 1.00 | 41.37 | C |
| ATOM | 2076 | C | GLY | A | 280 | 35.890 | 62.801 | 60.589 | 1.00 | 43.18 | C |
| ATOM | 2077 | O | GLY | A | 280 | 37.117 | 62.761 | 60.689 | 1.00 | 42.71 | O |
| ATOM | 2078 | N | GLU | A | 281 | 35.156 | 63.763 | 61.144 | 1.00 | 45.40 | N |
| ATOM | 2079 | CA | GLU | A | 281 | 35.798 | 64.835 | 61.894 | 1.00 | 47.81 | C |
| ATOM | 2080 | CB | GLU | A | 281 | 34.787 | 65.930 | 62.253 | 1.00 | 50.66 | C |
| ATOM | 2081 | CG | GLU | A | 281 | 34.359 | 66.817 | 61.064 | 1.00 | 55.85 | C |
| ATOM | 2082 | CD | GLU | A | 281 | 35.540 | 67.416 | 60.290 | 1.00 | 57.87 | C |
| ATOM | 2083 | OE1 | GLU | A | 281 | 36.461 | 67.974 | 60.929 | 1.00 | 60.74 | O |
| ATOM | 2084 | OE2 | GLU | A | 281 | 35.544 | 67.338 | 59.043 | 1.00 | 58.63 | O |
| ATOM | 2085 | C | GLU | A | 281 | 36.527 | 64.357 | 63.147 | 1.00 | 47.68 | C |
| ATOM | 2086 | O | GLU | A | 281 | 37.523 | 64.954 | 63.543 | 1.00 | 48.52 | O |
| ATOM | 2087 | N | GLU | A | 282 | 36.040 | 63.297 | 63.786 | 1.00 | 47.14 | N |
| ATOM | 2088 | CA | GLU | A | 282 | 36.740 | 62.791 | 64.959 | 1.00 | 46.03 | C |
| ATOM | 2089 | CB | GLU | A | 282 | 36.024 | 63.187 | 66.252 | 1.00 | 46.80 | C |
| ATOM | 2090 | CG | GLU | A | 282 | 34.615 | 62.731 | 66.379 | 1.00 | 48.14 | C |
| ATOM | 2091 | CD | GLU | A | 282 | 34.040 | 63.106 | 67.730 | 1.00 | 49.14 | C |
| ATOM | 2092 | OE1 | GLU | A | 282 | 34.433 | 62.477 | 68.736 | 1.00 | 49.18 | O |
| ATOM | 2093 | OE2 | GLU | A | 282 | 33.205 | 64.035 | 67.792 | 1.00 | 49.39 | O |
| ATOM | 2094 | C | GLU | A | 282 | 37.004 | 61.289 | 64.953 | 1.00 | 44.47 | C |
| ATOM | 2095 | O | GLU | A | 282 | 37.866 | 60.808 | 65.692 | 1.00 | 44.09 | O |
| ATOM | 2096 | N | ASP | A | 283 | 36.275 | 60.541 | 64.133 | 1.00 | 41.60 | N |
| ATOM | 2097 | CA | ASP | A | 283 | 36.513 | 59.103 | 64.064 | 1.00 | 39.06 | C |
| ATOM | 2098 | CB | ASP | A | 283 | 35.219 | 58.288 | 64.109 | 1.00 | 37.77 | C |
| ATOM | 2099 | CG | ASP | A | 283 | 34.473 | 58.396 | 65.416 | 1.00 | 36.52 | C |
| ATOM | 2100 | OD1 | ASP | A | 283 | 35.036 | 58.872 | 66.424 | 1.00 | 35.74 | O |
| ATOM | 2101 | OD2 | ASP | A | 283 | 33.298 | 57.961 | 65.411 | 1.00 | 36.21 | O |
| ATOM | 2102 | C | ASP | A | 283 | 37.184 | 58.721 | 62.760 | 1.00 | 38.92 | C |
| ATOM | 2103 | O | ASP | A | 283 | 37.006 | 59.374 | 61.725 | 1.00 | 38.09 | O |
| ATOM | 2104 | N | LEU | A | 284 | 37.966 | 57.652 | 62.827 | 1.00 | 37.62 | N |
| ATOM | 2105 | CA | LEU | A | 284 | 38.596 | 57.072 | 61.652 | 1.00 | 36.11 | C |
| ATOM | 2106 | CB | LEU | A | 284 | 40.112 | 57.144 | 61.738 | 1.00 | 37.03 | C |
| ATOM | 2107 | CG | LEU | A | 284 | 40.775 | 56.919 | 60.375 | 1.00 | 38.44 | C |
| ATOM | 2108 | CD1 | LEU | A | 284 | 42.251 | 57.333 | 60.445 | 1.00 | 38.90 | C |
| ATOM | 2109 | CD2 | LEU | A | 284 | 40.637 | 55.456 | 59.969 | 1.00 | 38.70 | C |
| ATOM | 2110 | C | LEU | A | 284 | 38.079 | 55.632 | 61.777 | 1.00 | 34.72 | C |
| ATOM | 2111 | O | LEU | A | 284 | 38.499 | 54.875 | 62.652 | 1.00 | 33.63 | O |
| ATOM | 2112 | N | SER | A | 285 | 37.131 | 55.275 | 60.925 | 1.00 | 33.69 | N |
| ATOM | 2113 | CA | SER | A | 285 | 36.514 | 53.967 | 61.013 | 1.00 | 34.32 | C |
| ATOM | 2114 | CB | SER | A | 285 | 35.002 | 54.137 | 61.056 | 1.00 | 32.85 | C |
| ATOM | 2115 | OG | SER | A | 285 | 34.653 | 55.112 | 62.023 | 1.00 | 34.64 | O |
| ATOM | 2116 | C | SER | A | 285 | 36.892 | 52.999 | 59.918 | 1.00 | 34.01 | C |
| ATOM | 2117 | O | SER | A | 285 | 36.986 | 53.367 | 58.748 | 1.00 | 33.67 | O |
| ATOM | 2118 | N | ILE | A | 286 | 37.103 | 51.746 | 60.312 | 1.00 | 33.96 | N |
| ATOM | 2119 | CA | ILE | A | 286 | 37.466 | 50.701 | 59.366 | 1.00 | 35.20 | C |
| ATOM | 2120 | CB | ILE | A | 286 | 38.892 | 50.168 | 59.631 | 1.00 | 35.06 | C |
| ATOM | 2121 | CG2 | ILE | A | 286 | 39.292 | 49.199 | 58.547 | 1.00 | 34.26 | C |
| ATOM | 2122 | CG1 | ILE | A | 286 | 39.885 | 51.332 | 59.662 | 1.00 | 36.94 | C |
| ATOM | 2123 | CD1 | ILE | A | 286 | 41.260 | 50.964 | 60.181 | 1.00 | 36.59 | C |
| ATOM | 2124 | C | ILE | A | 286 | 36.489 | 49.549 | 59.494 | 1.00 | 35.03 | C |
| ATOM | 2125 | O | ILE | A | 286 | 36.459 | 48.864 | 60.515 | 1.00 | 34.85 | O |
| ATOM | 2126 | N | LYS | A | 287 | 35.687 | 49.342 | 58.455 | 1.00 | 35.73 | N |
| ATOM | 2127 | CA | LYS | A | 287 | 34.727 | 48.250 | 58.466 | 1.00 | 36.64 | C |
| ATOM | 2128 | CB | LYS | A | 287 | 33.341 | 48.731 | 57.992 | 1.00 | 36.83 | C |
| ATOM | 2129 | CG | LYS | A | 287 | 32.238 | 47.674 | 58.147 | 1.00 | 39.09 | C |
| ATOM | 2130 | CD | LYS | A | 287 | 30.848 | 48.302 | 58.277 | 1.00 | 42.04 | C |
| ATOM | 2131 | CE | LYS | A | 287 | 30.253 | 48.597 | 56.912 | 1.00 | 46.63 | C |
| ATOM | 2132 | NZ | LYS | A | 287 | 29.252 | 49.718 | 56.920 | 1.00 | 49.07 | N |
| ATOM | 2133 | C | LYS | A | 287 | 35.214 | 47.107 | 57.579 | 1.00 | 35.89 | C |
| ATOM | 2134 | O | LYS | A | 287 | 35.682 | 47.324 | 56.471 | 1.00 | 36.89 | O |
| ATOM | 2135 | N | MET | A | 288 | 35.128 | 45.889 | 58.086 | 1.00 | 36.29 | N |
| ATOM | 2136 | CA | MET | A | 288 | 35.529 | 44.723 | 57.315 | 1.00 | 36.06 | C |
| ATOM | 2137 | CB | MET | A | 288 | 36.631 | 43.943 | 58.021 | 1.00 | 35.41 | C |
| ATOM | 2138 | CG | MET | A | 288 | 37.133 | 42.753 | 57.217 | 1.00 | 34.77 | C |
| ATOM | 2139 | SD | MET | A | 288 | 38.277 | 41.740 | 58.141 | 1.00 | 36.38 | S |
| ATOM | 2140 | CE | MET | A | 288 | 39.704 | 42.912 | 58.320 | 1.00 | 32.85 | C |
| ATOM | 2141 | C | MET | A | 288 | 34.291 | 43.847 | 57.192 | 1.00 | 36.94 | C |
| ATOM | 2142 | O | MET | A | 288 | 33.821 | 43.297 | 58.189 | 1.00 | 36.54 | O |
| ATOM | 2143 | N | SER | A | 289 | 33.778 | 43.721 | 55.969 | 1.00 | 37.24 | N |
| ATOM | 2144 | CA | SER | A | 289 | 32.571 | 42.945 | 55.703 | 1.00 | 38.61 | C |
| ATOM | 2145 | CB | SER | A | 289 | 31.607 | 43.785 | 54.860 | 1.00 | 38.11 | C |
| ATOM | 2146 | OG | SER | A | 289 | 31.226 | 44.948 | 55.569 | 1.00 | 39.06 | O |
| ATOM | 2147 | C | SER | A | 289 | 32.806 | 41.617 | 55.007 | 1.00 | 38.93 | C |
| ATOM | 2148 | O | SER | A | 289 | 33.514 | 41.559 | 54.009 | 1.00 | 39.37 | O |
| ATOM | 2149 | N | ASP | A | 290 | 32.196 | 40.560 | 55.535 | 1.00 | 38.97 | N |
| ATOM | 2150 | CA | ASP | A | 290 | 32.307 | 39.245 | 54.945 | 1.00 | 40.26 | C |
| ATOM | 2151 | CB | ASP | A | 290 | 33.202 | 38.344 | 55.801 | 1.00 | 40.37 | C |
| ATOM | 2152 | CG | ASP | A | 290 | 32.582 | 37.987 | 57.147 | 1.00 | 40.75 | C |
| ATOM | 2153 | OD1 | ASP | A | 290 | 31.458 | 37.427 | 57.178 | 1.00 | 38.83 | O |
| ATOM | 2154 | OD2 | ASP | A | 290 | 33.240 | 38.257 | 58.176 | 1.00 | 41.70 | O |
| ATOM | 2155 | C | ASP | A | 290 | 30.941 | 38.582 | 54.783 | 1.00 | 41.90 | C |
| ATOM | 2156 | O | ASP | A | 290 | 29.936 | 39.049 | 55.326 | 1.00 | 41.33 | O |
| ATOM | 2157 | N | ARG | A | 291 | 30.926 | 37.488 | 54.027 | 1.00 | 42.75 | N |
| ATOM | 2158 | CA | ARG | A | 291 | 29.726 | 36.705 | 53.794 | 1.00 | 44.02 | C |
| ATOM | 2159 | CB | ARG | A | 291 | 29.331 | 36.747 | 52.323 | 1.00 | 46.86 | C |
| ATOM | 2160 | CG | ARG | A | 291 | 28.960 | 38.130 | 51.878 | 1.00 | 50.34 | C |
| ATOM | 2161 | CD | ARG | A | 291 | 28.514 | 38.184 | 50.438 | 1.00 | 56.07 | C |
| ATOM | 2162 | NE | ARG | A | 291 | 28.035 | 39.531 | 50.133 | 1.00 | 61.19 | N |
| ATOM | 2163 | CZ | ARG | A | 291 | 28.778 | 40.633 | 50.222 | 1.00 | 63.26 | C |
| ATOM | 2164 | NH1 | ARG | A | 291 | 30.055 | 40.562 | 50.601 | 1.00 | 64.36 | N |
| ATOM | 2165 | NH2 | ARG | A | 291 | 28.234 | 41.816 | 49.953 | 1.00 | 65.22 | N |
| ATOM | 2166 | C | ARG | A | 291 | 30.057 | 35.286 | 54.210 | 1.00 | 42.53 | C |
| ATOM | 2167 | O | ARG | A | 291 | 29.877 | 34.341 | 53.444 | 1.00 | 42.69 | O |
| ATOM | 2168 | N | GLY | A | 292 | 30.556 | 35.165 | 55.436 | 1.00 | 41.43 | N |
| ATOM | 2169 | CA | GLY | A | 292 | 30.933 | 33.876 | 55.981 | 1.00 | 40.59 | C |
| ATOM | 2170 | C | GLY | A | 292 | 29.823 | 33.218 | 56.767 | 1.00 | 40.13 | C |
| ATOM | 2171 | O | GLY | A | 292 | 30.092 | 32.444 | 57.687 | 1.00 | 39.97 | O |
| ATOM | 2172 | N | GLY | A | 293 | 28.576 | 33.522 | 56.410 | 1.00 | 40.41 | N |
| ATOM | 2173 | CA | GLY | A | 293 | 27.444 | 32.921 | 57.099 | 1.00 | 41.38 | C |
| ATOM | 2174 | C | GLY | A | 293 | 26.964 | 33.653 | 58.340 | 1.00 | 41.62 | C |
| ATOM | 2175 | O | GLY | A | 293 | 25.845 | 33.431 | 58.792 | 1.00 | 42.98 | O |
| ATOM | 2176 | N | GLY | A | 294 | 27.797 | 34.512 | 58.908 | 1.00 | 40.63 | N |
| ATOM | 2177 | CA | GLY | A | 294 | 27.365 | 35.242 | 60.080 | 1.00 | 41.89 | C |
| ATOM | 2178 | C | GLY | A | 294 | 27.343 | 34.410 | 61.343 | 1.00 | 43.13 | C |
| ATOM | 2179 | O | GLY | A | 294 | 27.704 | 33.229 | 61.336 | 1.00 | 45.03 | O |
| ATOM | 2180 | N | VAL | A | 295 | 26.900 | 35.034 | 62.429 | 1.00 | 42.25 | N |
| ATOM | 2181 | CA | VAL | A | 295 | 26.839 | 34.411 | 63.746 | 1.00 | 40.95 | C |
| ATOM | 2182 | CB | VAL | A | 295 | 28.044 | 34.870 | 64.593 | 1.00 | 41.89 | C |
| ATOM | 2183 | CG1 | VAL | A | 295 | 27.929 | 34.356 | 66.017 | 1.00 | 40.89 | C |
| ATOM | 2184 | CG2 | VAL | A | 295 | 29.326 | 34.404 | 63.943 | 1.00 | 42.58 | C |
| ATOM | 2185 | C | VAL | A | 295 | 25.553 | 34.838 | 64.464 | 1.00 | 41.64 | C |
| ATOM | 2186 | O | VAL | A | 295 | 25.136 | 35.980 | 64.354 | 1.00 | 41.25 | O |
| ATOM | 2187 | N | PRO | A | 296 | 24.908 | 33.917 | 65.204 | 1.00 | 42.14 | N |
| ATOM | 2188 | CD | PRO | A | 296 | 25.320 | 32.515 | 65.387 | 1.00 | 40.62 | C |
| ATOM | 2189 | CA | PRO | A | 296 | 23.671 | 34.200 | 65.946 | 1.00 | 40.95 | C |
| ATOM | 2190 | CB | PRO | A | 296 | 23.364 | 32.863 | 66.629 | 1.00 | 41.53 | C |
| ATOM | 2191 | CG | PRO | A | 296 | 24.046 | 31.866 | 65.775 | 1.00 | 41.49 | C |
| ATOM | 2192 | C | PRO | A | 296 | 23.949 | 35.298 | 66.983 | 1.00 | 41.76 | C |
| ATOM | 2193 | O | PRO | A | 296 | 24.916 | 35.189 | 67.751 | 1.00 | 41.34 | O |
| ATOM | 2194 | N | LEU | A | 297 | 23.108 | 36.331 | 67.026 | 1.00 | 41.50 | N |
| ATOM | 2195 | CA | LEU | A | 297 | 23.306 | 37.432 | 67.973 | 1.00 | 41.81 | C |
| ATOM | 2196 | CB | LEU | A | 297 | 22.049 | 38.297 | 68.087 | 1.00 | 40.31 | C |
| ATOM | 2197 | CG | LEU | A | 297 | 22.209 | 39.435 | 69.116 | 1.00 | 39.20 | C |
| ATOM | 2198 | CD1 | LEU | A | 297 | 23.323 | 40.391 | 68.673 | 1.00 | 36.12 | C |
| ATOM | 2199 | CD2 | LEU | A | 297 | 20.881 | 40.170 | 69.292 | 1.00 | 38.63 | C |
| ATOM | 2200 | C | LEU | A | 297 | 23.692 | 36.975 | 69.375 | 1.00 | 42.65 | C |
| ATOM | 2201 | O | LEU | A | 297 | 24.500 | 37.597 | 70.053 | 1.00 | 41.79 | O |
| ATOM | 2202 | N | ARG | A | 298 | 23.098 | 35.879 | 69.809 | 1.00 | 45.74 | N |
| ATOM | 2203 | CA | ARG | A | 298 | 23.364 | 35.329 | 71.130 | 1.00 | 47.01 | C |
| ATOM | 2204 | CB | ARG | A | 298 | 22.351 | 34.224 | 71.390 | 1.00 | 49.32 | C |
| ATOM | 2205 | CG | ARG | A | 298 | 22.742 | 33.234 | 72.440 | 1.00 | 52.35 | C |
| ATOM | 2206 | CD | ARG | A | 298 | 21.814 | 32.059 | 72.366 | 1.00 | 54.85 | C |
| ATOM | 2207 | NE | ARG | A | 298 | 22.563 | 30.815 | 72.295 | 1.00 | 58.47 | N |
| ATOM | 2208 | CZ | ARG | A | 298 | 22.476 | 29.954 | 71.291 | 1.00 | 59.82 | C |
| ATOM | 2209 | NH1 | ARG | A | 298 | 21.669 | 30.205 | 70.272 | 1.00 | 60.60 | N |
| ATOM | 2210 | NH2 | ARG | A | 298 | 23.191 | 28.838 | 71.313 | 1.00 | 62.15 | N |
| ATOM | 2211 | C | ARG | A | 298 | 24.792 | 34.809 | 71.363 | 1.00 | 46.74 | C |
| ATOM | 2212 | O | ARG | A | 298 | 25.265 | 34.813 | 72.495 | 1.00 | 46.55 | O |
| ATOM | 2213 | N | LYS | A | 299 | 25.466 | 34.354 | 70.312 | 1.00 | 48.55 | N |
| ATOM | 2214 | CA | LYS | A | 299 | 26.834 | 33.855 | 70.452 | 1.00 | 50.97 | C |
| ATOM | 2215 | CB | LYS | A | 299 | 27.116 | 32.735 | 69.437 | 1.00 | 51.63 | C |
| ATOM | 2216 | CG | LYS | A | 299 | 26.390 | 31.415 | 69.701 | 1.00 | 54.50 | C |
| ATOM | 2217 | CD | LYS | A | 299 | 26.701 | 30.370 | 68.613 | 1.00 | 56.33 | C |
| ATOM | 2218 | CE | LYS | A | 299 | 25.846 | 29.113 | 68.791 | 1.00 | 58.10 | C |
| ATOM | 2219 | NZ | LYS | A | 299 | 25.790 | 28.244 | 67.566 | 1.00 | 59.46 | N |
| ATOM | 2220 | C | LYS | A | 299 | 27.853 | 34.983 | 70.254 | 1.00 | 51.90 | C |
| ATOM | 2221 | O | LYS | A | 299 | 29.011 | 34.868 | 70.658 | 1.00 | 51.49 | O |
| ATOM | 2222 | N | ILE | A | 300 | 27.416 | 36.070 | 69.621 | 1.00 | 54.19 | N |
| ATOM | 2223 | CA | ILE | A | 300 | 28.284 | 37.217 | 69.355 | 1.00 | 56.34 | C |
| ATOM | 2224 | CB | ILE | A | 300 | 27.500 | 38.347 | 68.632 | 1.00 | 56.02 | C |
| ATOM | 2225 | CG2 | ILE | A | 300 | 28.208 | 39.671 | 68.805 | 1.00 | 55.50 | C |
| ATOM | 2226 | CG1 | ILE | A | 300 | 27.358 | 38.015 | 67.145 | 1.00 | 55.93 | C |
| ATOM | 2227 | CD1 | ILE | A | 300 | 28.694 | 37.849 | 66.414 | 1.00 | 55.49 | C |
| ATOM | 2228 | C | ILE | A | 300 | 28.886 | 37.760 | 70.645 | 1.00 | 57.58 | C |
| ATOM | 2229 | O | ILE | A | 300 | 30.064 | 38.118 | 70.703 | 1.00 | 56.60 | O |
| ATOM | 2230 | N | GLU | A | 301 | 28.055 | 37.810 | 71.674 | 1.00 | 60.07 | N |
| ATOM | 2231 | CA | GLU | A | 301 | 28.446 | 38.287 | 72.993 | 1.00 | 62.73 | C |
| ATOM | 2232 | CB | GLU | A | 301 | 27.235 | 38.185 | 73.921 | 1.00 | 66.58 | C |
| ATOM | 2233 | CG | GLU | A | 301 | 27.399 | 38.825 | 75.290 | 1.00 | 71.08 | C |
| ATOM | 2234 | CD | GLU | A | 301 | 27.324 | 40.347 | 75.248 | 1.00 | 73.52 | C |
| ATOM | 2235 | OE1 | GLU | A | 301 | 26.978 | 40.913 | 74.181 | 1.00 | 74.00 | O |
| ATOM | 2236 | OE2 | GLU | A | 301 | 27.606 | 40.974 | 76.293 | 1.00 | 75.20 | O |
| ATOM | 2237 | C | GLU | A | 301 | 29.607 | 37.470 | 73.574 | 1.00 | 62.65 | C |
| ATOM | 2238 | O | GLU | A | 301 | 30.523 | 38.017 | 74.193 | 1.00 | 62.99 | O |
| ATOM | 2239 | N | ARG | A | 302 | 29.566 | 36.159 | 73.362 | 1.00 | 61.68 | N |
| ATOM | 2240 | CA | ARG | A | 302 | 30.591 | 35.262 | 73.886 | 1.00 | 61.14 | C |
| ATOM | 2241 | CB | ARG | A | 302 | 30.051 | 33.829 | 73.904 | 1.00 | 63.88 | C |
| ATOM | 2242 | CG | ARG | A | 302 | 28.683 | 33.726 | 74.551 | 1.00 | 68.42 | C |
| ATOM | 2243 | CD | ARG | A | 302 | 28.181 | 32.302 | 74.688 | 1.00 | 71.42 | C |
| ATOM | 2244 | NE | ARG | A | 302 | 27.551 | 32.123 | 75.994 | 1.00 | 74.63 | N |
| ATOM | 2245 | CZ | ARG | A | 302 | 28.032 | 31.339 | 76.954 | 1.00 | 75.82 | C |
| ATOM | 2246 | NH1 | ARG | A | 302 | 29.148 | 30.651 | 76.748 | 1.00 | 75.79 | N |
| ATOM | 2247 | NH2 | ARG | A | 302 | 27.412 | 31.262 | 78.130 | 1.00 | 76.46 | N |
| ATOM | 2248 | C | ARG | A | 302 | 31.910 | 35.298 | 73.126 | 1.00 | 59.13 | C |
| ATOM | 2249 | O | ARG | A | 302 | 32.931 | 34.840 | 73.635 | 1.00 | 58.52 | O |
| ATOM | 2250 | N | LEU | A | 303 | 31.891 | 35.851 | 71.918 | 1.00 | 57.00 | N |
| ATOM | 2251 | CA | LEU | A | 303 | 33.091 | 35.913 | 71.094 | 1.00 | 54.37 | C |
| ATOM | 2252 | CB | LEU | A | 303 | 32.761 | 36.458 | 69.699 | 1.00 | 54.07 | C |
| ATOM | 2253 | CG | LEU | A | 303 | 31.776 | 35.632 | 68.870 | 1.00 | 52.51 | C |
| ATOM | 2254 | CD1 | LEU | A | 303 | 31.752 | 36.154 | 67.453 | 1.00 | 52.04 | C |
| ATOM | 2255 | CD2 | LEU | A | 303 | 32.191 | 34.177 | 68.894 | 1.00 | 52.43 | C |
| ATOM | 2256 | C | LEU | A | 303 | 34.221 | 36.726 | 71.695 | 1.00 | 52.67 | C |
| ATOM | 2257 | O | LEU | A | 303 | 35.383 | 36.481 | 71.386 | 1.00 | 53.26 | O |
| ATOM | 2258 | N | PHE | A | 304 | 33.899 | 37.697 | 72.542 | 1.00 | 51.08 | N |
| ATOM | 2259 | CA | PHE | A | 304 | 34.945 | 38.509 | 73.157 | 1.00 | 50.58 | C |
| ATOM | 2260 | CB | PHE | A | 304 | 34.528 | 39.986 | 73.211 | 1.00 | 46.75 | C |
| ATOM | 2261 | CG | PHE | A | 304 | 34.466 | 40.661 | 71.858 | 1.00 | 44.57 | C |
| ATOM | 2262 | CD1 | PHE | A | 304 | 33.313 | 40.603 | 71.081 | 1.00 | 42.21 | C |
| ATOM | 2263 | CD2 | PHE | A | 304 | 35.556 | 41.379 | 71.375 | 1.00 | 42.80 | C |
| ATOM | 2264 | CE1 | PHE | A | 304 | 33.248 | 41.249 | 69.855 | 1.00 | 42.14 | C |
| ATOM | 2265 | CE2 | PHE | A | 304 | 35.497 | 42.030 | 70.144 | 1.00 | 41.90 | C |
| ATOM | 2266 | CZ | PHE | A | 304 | 34.341 | 41.968 | 69.384 | 1.00 | 40.81 | C |
| ATOM | 2267 | C | PHE | A | 304 | 35.294 | 38.027 | 74.569 | 1.00 | 51.49 | C |
| ATOM | 2268 | O | PHE | A | 304 | 36.013 | 38.701 | 75.306 | 1.00 | 51.13 | O |
| ATOM | 2269 | N | SER | A | 305 | 34.789 | 36.853 | 74.935 | 1.00 | 52.86 | N |
| ATOM | 2270 | CA | SER | A | 305 | 35.035 | 36.292 | 76.260 | 1.00 | 55.26 | C |
| ATOM | 2271 | CB | SER | A | 305 | 33.842 | 35.450 | 76.708 | 1.00 | 55.58 | C |
| ATOM | 2272 | OG | SER | A | 305 | 34.159 | 34.742 | 77.893 | 1.00 | 57.49 | O |
| ATOM | 2273 | C | SER | A | 305 | 36.288 | 35.437 | 76.319 | 1.00 | 56.01 | C |
| ATOM | 2274 | O | SER | A | 305 | 36.477 | 34.548 | 75.503 | 1.00 | 55.44 | O |
| ATOM | 2275 | N | TYR | A | 306 | 37.149 | 35.711 | 77.288 | 1.00 | 57.91 | N |
| ATOM | 2276 | CA | TYR | A | 306 | 38.374 | 34.939 | 77.433 | 1.00 | 61.22 | C |
| ATOM | 2277 | CB | TYR | A | 306 | 39.279 | 35.574 | 78.485 | 1.00 | 59.00 | C |
| ATOM | 2278 | CG | TYR | A | 306 | 39.881 | 36.890 | 78.069 | 1.00 | 57.14 | C |
| ATOM | 2279 | CD1 | TYR | A | 306 | 40.942 | 36.936 | 77.167 | 1.00 | 55.23 | C |
| ATOM | 2280 | CE1 | TYR | A | 306 | 41.497 | 38.146 | 76.783 | 1.00 | 55.64 | C |
| ATOM | 2281 | CD2 | TYR | A | 306 | 39.387 | 38.094 | 78.579 | 1.00 | 56.00 | C |
| ATOM | 2282 | CE2 | TYR | A | 306 | 39.935 | 39.309 | 78.204 | 1.00 | 56.02 | C |
| ATOM | 2283 | CZ | TYR | A | 306 | 40.986 | 39.332 | 77.308 | 1.00 | 56.17 | C |
| ATOM | 2284 | OH | TYR | A | 306 | 41.506 | 40.552 | 76.955 | 1.00 | 56.57 | O |
| ATOM | 2285 | C | TYR | A | 306 | 38.012 | 33.527 | 77.880 | 1.00 | 64.47 | C |
| ATOM | 2286 | O | TYR | A | 306 | 38.521 | 32.539 | 77.353 | 1.00 | 63.92 | O |
| ATOM | 2287 | N | MET | A | 307 | 37.114 | 33.447 | 78.856 | 1.00 | 69.01 | N |
| ATOM | 2288 | CA | MET | A | 307 | 36.696 | 32.168 | 79.404 | 1.00 | 73.45 | C |
| ATOM | 2289 | CB | MET | A | 307 | 35.769 | 32.386 | 80.596 | 1.00 | 75.03 | C |
| ATOM | 2290 | CG | MET | A | 307 | 35.483 | 31.112 | 81.364 | 1.00 | 77.78 | C |
| ATOM | 2291 | SD | MET | A | 307 | 34.655 | 31.463 | 82.914 | 1.00 | 81.39 | S |
| ATOM | 2292 | CE | MET | A | 307 | 32.948 | 31.372 | 82.405 | 1.00 | 79.34 | C |
| ATOM | 2293 | C | MET | A | 307 | 36.015 | 31.260 | 78.390 | 1.00 | 75.39 | C |
| ATOM | 2294 | O | MET | A | 307 | 36.433 | 30.115 | 78.192 | 1.00 | 75.64 | O |
| ATOM | 2295 | N | TYR | A | 308 | 34.968 | 31.765 | 77.746 | 1.00 | 77.45 | N |
| ATOM | 2296 | CA | TYR | A | 308 | 34.244 | 30.966 | 76.769 | 1.00 | 79.30 | C |
| ATOM | 2297 | CB | TYR | A | 308 | 32.910 | 31.639 | 76.428 | 1.00 | 79.88 | C |
| ATOM | 2298 | CG | TYR | A | 308 | 31.996 | 31.825 | 77.626 | 1.00 | 80.36 | C |
| ATOM | 2299 | CD1 | TYR | A | 308 | 31.431 | 33.071 | 77.906 | 1.00 | 80.88 | C |
| ATOM | 2300 | CE1 | TYR | A | 308 | 30.602 | 33.263 | 79.008 | 1.00 | 80.99 | C |
| ATOM | 2301 | CD2 | TYR | A | 308 | 31.702 | 30.763 | 78.484 | 1.00 | 80.80 | C |
| ATOM | 2302 | CE2 | TYR | A | 308 | 30.867 | 30.943 | 79.592 | 1.00 | 81.61 | C |
| ATOM | 2303 | CZ | TYR | A | 308 | 30.322 | 32.199 | 79.847 | 1.00 | 81.53 | C |
| ATOM | 2304 | OH | TYR | A | 308 | 29.506 | 32.398 | 80.941 | 1.00 | 82.05 | O |
| ATOM | 2305 | C | TYR | A | 308 | 35.059 | 30.714 | 75.499 | 1.00 | 80.21 | C |
| ATOM | 2306 | O | TYR | A | 308 | 34.816 | 29.738 | 74.789 | 1.00 | 80.57 | O |
| ATOM | 2307 | N | SER | A | 309 | 36.037 | 31.571 | 75.217 | 1.00 | 81.22 | N |
| ATOM | 2308 | CA | SER | A | 309 | 36.849 | 31.385 | 74.019 | 1.00 | 82.52 | C |
| ATOM | 2309 | CB | SER | A | 309 | 37.215 | 32.739 | 73.392 | 1.00 | 83.02 | C |
| ATOM | 2310 | OG | SER | A | 309 | 38.162 | 33.451 | 74.171 | 1.00 | 83.95 | O |
| ATOM | 2311 | C | SER | A | 309 | 38.122 | 30.576 | 74.278 | 1.00 | 83.15 | C |
| ATOM | 2312 | O | SER | A | 309 | 39.050 | 30.601 | 73.471 | 1.00 | 83.36 | O |
| ATOM | 2313 | N | THR | A | 310 | 38.165 | 29.848 | 75.393 | 1.00 | 83.92 | N |
| ATOM | 2314 | CA | THR | A | 310 | 39.337 | 29.033 | 75.723 | 1.00 | 84.39 | C |
| ATOM | 2315 | CB | THR | A | 310 | 39.984 | 29.467 | 77.071 | 1.00 | 84.75 | C |
| ATOM | 2316 | OG1 | THR | A | 310 | 40.360 | 30.849 | 77.007 | 1.00 | 85.50 | O |
| ATOM | 2317 | CG2 | THR | A | 310 | 41.234 | 28.638 | 77.358 | 1.00 | 84.97 | C |
| ATOM | 2318 | C | THR | A | 310 | 38.975 | 27.551 | 75.813 | 1.00 | 84.17 | C |
| ATOM | 2319 | O | THR | A | 310 | 37.943 | 27.185 | 76.375 | 1.00 | 84.14 | O |
| ATOM | 2320 | N | LEU | A | 323 | 44.333 | 28.618 | 61.136 | 1.00 | 86.34 | N |
| ATOM | 2321 | CA | LEU | A | 323 | 44.370 | 30.052 | 61.390 | 1.00 | 86.52 | C |
| ATOM | 2322 | CB | LEU | A | 323 | 43.367 | 30.787 | 60.490 | 1.00 | 86.02 | C |
| ATOM | 2323 | CG | LEU | A | 323 | 43.394 | 30.635 | 58.974 | 1.00 | 85.96 | C |
| ATOM | 2324 | CD1 | LEU | A | 323 | 41.983 | 30.764 | 58.428 | 1.00 | 85.55 | C |
| ATOM | 2325 | CD2 | LEU | A | 323 | 44.308 | 31.678 | 58.360 | 1.00 | 86.00 | C |
| ATOM | 2326 | C | LEU | A | 323 | 44.009 | 30.380 | 62.842 | 1.00 | 86.38 | C |
| ATOM | 2327 | O | LEU | A | 323 | 44.623 | 31.253 | 63.471 | 1.00 | 86.39 | O |
| ATOM | 2328 | N | ALA | A | 324 | 43.014 | 29.652 | 63.352 | 1.00 | 86.44 | N |
| ATOM | 2329 | CA | ALA | A | 324 | 42.432 | 29.830 | 64.689 | 1.00 | 86.57 | C |
| ATOM | 2330 | CB | ALA | A | 324 | 40.910 | 29.804 | 64.542 | 1.00 | 86.19 | C |
| ATOM | 2331 | C | ALA | A | 324 | 42.818 | 28.900 | 65.856 | 1.00 | 86.64 | C |
| ATOM | 2332 | O | ALA | A | 324 | 42.981 | 27.699 | 65.671 | 1.00 | 86.60 | O |
| ATOM | 2333 | N | GLY | A | 325 | 42.919 | 29.475 | 67.055 | 1.00 | 86.41 | N |
| ATOM | 2334 | CA | GLY | A | 325 | 43.225 | 28.670 | 68.213 | 1.00 | 86.30 | C |
| ATOM | 2335 | C | GLY | A | 325 | 44.112 | 29.217 | 69.314 | 1.00 | 86.08 | C |
| ATOM | 2336 | O | GLY | A | 325 | 43.895 | 28.944 | 70.492 | 1.00 | 86.61 | O |
| ATOM | 2337 | N | PHE | A | 326 | 45.110 | 30.007 | 68.972 | 1.00 | 85.67 | N |
| ATOM | 2338 | CA | PHE | A | 326 | 45.978 | 30.563 | 69.986 | 1.00 | 84.76 | C |
| ATOM | 2339 | CB | PHE | A | 326 | 47.390 | 30.770 | 69.412 | 1.00 | 85.63 | C |
| ATOM | 2340 | CG | PHE | A | 326 | 47.427 | 31.281 | 68.007 | 1.00 | 86.77 | C |
| ATOM | 2341 | CD1 | PHE | A | 326 | 47.631 | 32.631 | 67.748 | 1.00 | 87.21 | C |
| ATOM | 2342 | CD2 | PHE | A | 326 | 47.231 | 30.408 | 66.937 | 1.00 | 87.27 | C |
| ATOM | 2343 | CE1 | PHE | A | 326 | 47.629 | 33.110 | 66.441 | 1.00 | 87.91 | C |
| ATOM | 2344 | CE2 | PHE | A | 326 | 47.226 | 30.875 | 65.627 | 1.00 | 87.35 | C |
| ATOM | 2345 | CZ | PHE | A | 326 | 47.423 | 32.229 | 65.385 | 1.00 | 87.91 | C |
| ATOM | 2346 | C | PHE | A | 326 | 45.390 | 31.857 | 70.519 | 1.00 | 83.23 | C |
| ATOM | 2347 | O | PHE | A | 326 | 44.765 | 31.898 | 71.585 | 1.00 | 83.76 | O |
| ATOM | 2348 | N | GLY | A | 327 | 45.574 | 32.905 | 69.748 | 1.00 | 81.07 | N |
| ATOM | 2349 | CA | GLY | A | 327 | 45.070 | 34.217 | 70.071 | 1.00 | 77.67 | C |
| ATOM | 2350 | C | GLY | A | 327 | 44.504 | 34.920 | 71.270 | 1.00 | 75.53 | C |
| ATOM | 2351 | O | GLY | A | 327 | 45.244 | 35.409 | 72.144 | 1.00 | 75.24 | O |
| ATOM | 2352 | N | TYR | A | 328 | 43.179 | 34.922 | 71.313 | 1.00 | 72.88 | N |
| ATOM | 2353 | CA | TYR | A | 328 | 42.420 | 35.677 | 72.290 | 1.00 | 69.47 | C |
| ATOM | 2354 | CB | TYR | A | 328 | 43.220 | 36.081 | 73.540 | 1.00 | 72.43 | C |
| ATOM | 2355 | CG | TYR | A | 328 | 43.270 | 34.922 | 74.505 | 1.00 | 75.02 | C |
| ATOM | 2356 | CD1 | TYR | A | 328 | 42.118 | 34.186 | 74.762 | 1.00 | 76.53 | C |
| ATOM | 2357 | CE1 | TYR | A | 328 | 42.143 | 33.069 | 75.554 | 1.00 | 78.23 | C |
| ATOM | 2358 | CD2 | TYR | A | 328 | 44.464 | 34.503 | 75.089 | 1.00 | 76.51 | C |
| ATOM | 2359 | CE2 | TYR | A | 328 | 44.509 | 33.378 | 75.894 | 1.00 | 77.99 | C |
| ATOM | 2360 | CZ | TYR | A | 328 | 43.330 | 32.661 | 76.117 | 1.00 | 78.74 | C |
| ATOM | 2361 | OH | TYR | A | 328 | 43.318 | 31.517 | 76.877 | 1.00 | 79.88 | O |
| ATOM | 2362 | C | TYR | A | 328 | 42.405 | 36.867 | 71.284 | 1.00 | 64.95 | C |
| ATOM | 2363 | O | TYR | A | 328 | 42.604 | 38.027 | 71.606 | 1.00 | 63.98 | O |
| ATOM | 2364 | N | GLY | A | 329 | 42.206 | 36.516 | 70.013 | 1.00 | 59.91 | N |
| ATOM | 2365 | CA | GLY | A | 329 | 42.180 | 37.416 | 68.861 | 1.00 | 54.13 | C |
| ATOM | 2366 | C | GLY | A | 329 | 41.253 | 38.600 | 69.025 | 1.00 | 50.06 | C |
| ATOM | 2367 | O | GLY | A | 329 | 41.686 | 39.757 | 68.995 | 1.00 | 49.83 | O |
| ATOM | 2368 | N | LEU | A | 330 | 39.956 | 38.347 | 69.129 | 1.00 | 46.27 | N |
| ATOM | 2369 | CA | LEU | A | 330 | 39.012 | 39.441 | 69.290 | 1.00 | 43.62 | C |
| ATOM | 2370 | CB | LEU | A | 330 | 37.581 | 38.925 | 69.127 | 1.00 | 42.23 | C |
| ATOM | 2371 | CG | LEU | A | 330 | 37.156 | 38.701 | 67.665 | 1.00 | 41.14 | C |
| ATOM | 2372 | CD1 | LEU | A | 330 | 35.873 | 37.874 | 67.631 | 1.00 | 38.93 | C |
| ATOM | 2373 | CD2 | LEU | A | 330 | 36.973 | 40.057 | 66.963 | 1.00 | 37.69 | C |
| ATOM | 2374 | C | LEU | A | 330 | 39.194 | 40.170 | 70.624 | 1.00 | 42.24 | C |
| ATOM | 2375 | O | LEU | A | 330 | 39.491 | 41.363 | 70.624 | 1.00 | 40.41 | O |
| ATOM | 2376 | N | PRO | A | 331 | 39.089 | 39.464 | 71.769 | 1.00 | 41.85 | N |
| ATOM | 2377 | CD | PRO | A | 331 | 39.010 | 38.020 | 72.075 | 1.00 | 39.94 | C |
| ATOM | 2378 | CA | PRO | A | 331 | 39.271 | 40.268 | 72.989 | 1.00 | 40.46 | C |
| ATOM | 2379 | CB | PRO | A | 331 | 39.178 | 39.234 | 74.121 | 1.00 | 40.60 | C |
| ATOM | 2380 | CG | PRO | A | 331 | 39.590 | 37.943 | 73.464 | 1.00 | 39.84 | C |
| ATOM | 2381 | C | PRO | A | 331 | 40.526 | 41.137 | 73.073 | 1.00 | 41.21 | C |
| ATOM | 2382 | O | PRO | A | 331 | 40.485 | 42.259 | 73.574 | 1.00 | 41.38 | O |
| ATOM | 2383 | N | ILE | A | 332 | 41.639 | 40.634 | 72.568 | 1.00 | 40.17 | N |
| ATOM | 2384 | CA | ILE | A | 332 | 42.852 | 41.413 | 72.626 | 1.00 | 39.04 | C |
| ATOM | 2385 | CB | ILE | A | 332 | 44.088 | 40.499 | 72.644 | 1.00 | 39.42 | C |
| ATOM | 2386 | CG2 | ILE | A | 332 | 45.358 | 41.304 | 72.373 | 1.00 | 38.07 | C |
| ATOM | 2387 | CG1 | ILE | A | 332 | 44.145 | 39.799 | 74.008 | 1.00 | 40.66 | C |
| ATOM | 2388 | CD1 | ILE | A | 332 | 45.446 | 39.082 | 74.285 | 1.00 | 40.56 | C |
| ATOM | 2389 | C | ILE | A | 332 | 42.941 | 42.454 | 71.514 | 1.00 | 38.68 | C |
| ATOM | 2390 | O | ILE | A | 332 | 43.499 | 43.530 | 71.715 | 1.00 | 38.26 | O |
| ATOM | 2391 | N | SER | A | 333 | 42.389 | 42.154 | 70.346 | 1.00 | 37.19 | N |
| ATOM | 2392 | CA | SER | A | 333 | 42.413 | 43.150 | 69.290 | 1.00 | 36.66 | C |
| ATOM | 2393 | CB | SER | A | 333 | 41.795 | 42.613 | 68.000 | 1.00 | 36.85 | C |
| ATOM | 2394 | OG | SER | A | 333 | 42.675 | 41.706 | 67.361 | 1.00 | 36.58 | O |
| ATOM | 2395 | C | SER | A | 333 | 41.606 | 44.336 | 69.808 | 1.00 | 35.99 | C |
| ATOM | 2396 | O | SER | A | 333 | 41.981 | 45.487 | 69.604 | 1.00 | 35.09 | O |
| ATOM | 2397 | N | ARG | A | 334 | 40.517 | 44.048 | 70.518 | 1.00 | 36.17 | N |
| ATOM | 2398 | CA | ARG | A | 334 | 39.678 | 45.102 | 71.056 | 1.00 | 35.16 | C |
| ATOM | 2399 | CB | ARG | A | 334 | 38.430 | 44.516 | 71.718 | 1.00 | 34.97 | C |
| ATOM | 2400 | CG | ARG | A | 334 | 37.437 | 45.584 | 72.146 | 1.00 | 36.07 | C |
| ATOM | 2401 | CD | ARG | A | 334 | 36.043 | 45.028 | 72.406 | 1.00 | 35.95 | C |
| ATOM | 2402 | NE | ARG | A | 334 | 35.991 | 44.256 | 73.643 | 1.00 | 38.14 | N |
| ATOM | 2403 | CZ | ARG | A | 334 | 34.900 | 43.661 | 74.123 | 1.00 | 38.01 | C |
| ATOM | 2404 | NH1 | ARG | A | 334 | 33.750 | 43.753 | 73.467 | 1.00 | 36.18 | N |
| ATOM | 2405 | NH2 | ARG | A | 334 | 34.968 | 42.959 | 75.255 | 1.00 | 37.95 | N |
| ATOM | 2406 | C | ARG | A | 334 | 40.425 | 45.989 | 72.050 | 1.00 | 35.30 | C |
| ATOM | 2407 | O | ARG | A | 334 | 40.289 | 47.216 | 72.035 | 1.00 | 33.89 | O |
| ATOM | 2408 | N | LEU | A | 335 | 41.210 | 45.366 | 72.922 | 1.00 | 35.05 | N |
| ATOM | 2409 | CA | LEU | A | 335 | 41.969 | 46.125 | 73.898 | 1.00 | 33.80 | C |
| ATOM | 2410 | CB | LEU | A | 335 | 42.764 | 45.183 | 74.810 | 1.00 | 34.47 | C |
| ATOM | 2411 | CG | LEU | A | 335 | 41.867 | 44.371 | 75.768 | 1.00 | 35.00 | C |
| ATOM | 2412 | CD1 | LEU | A | 335 | 42.729 | 43.531 | 76.692 | 1.00 | 33.39 | C |
| ATOM | 2413 | CD2 | LEU | A | 335 | 40.985 | 45.302 | 76.584 | 1.00 | 34.15 | C |
| ATOM | 2414 | C | LEU | A | 335 | 42.884 | 47.110 | 73.169 | 1.00 | 33.57 | C |
| ATOM | 2415 | O | LEU | A | 335 | 43.014 | 48.260 | 73.586 | 1.00 | 33.72 | O |
| ATOM | 2416 | N | TYR | A | 336 | 43.495 | 46.676 | 72.068 | 1.00 | 33.76 | N |
| ATOM | 2417 | CA | TYR | A | 336 | 44.362 | 47.566 | 71.302 | 1.00 | 35.02 | C |
| ATOM | 2418 | CB | TYR | A | 336 | 45.018 | 46.832 | 70.132 | 1.00 | 36.66 | C |
| ATOM | 2419 | CG | TYR | A | 336 | 46.258 | 46.076 | 70.500 | 1.00 | 39.90 | C |
| ATOM | 2420 | CD1 | TYR | A | 336 | 46.302 | 44.685 | 70.394 | 1.00 | 40.39 | C |
| ATOM | 2421 | CE1 | TYR | A | 336 | 47.449 | 43.975 | 70.727 | 1.00 | 39.75 | C |
| ATOM | 2422 | CD2 | TYR | A | 336 | 47.401 | 46.745 | 70.951 | 1.00 | 40.35 | C |
| ATOM | 2423 | CE2 | TYR | A | 336 | 48.554 | 46.043 | 71.288 | 1.00 | 40.18 | C |
| ATOM | 2424 | CZ | TYR | A | 336 | 48.565 | 44.654 | 71.173 | 1.00 | 40.65 | C |
| ATOM | 2425 | OH | TYR | A | 336 | 49.690 | 43.939 | 71.507 | 1.00 | 41.60 | O |
| ATOM | 2426 | C | TYR | A | 336 | 43.593 | 48.760 | 70.752 | 1.00 | 34.00 | C |
| ATOM | 2427 | O | TYR | A | 336 | 44.138 | 49.858 | 70.648 | 1.00 | 34.60 | O |
| ATOM | 2428 | N | ALA | A | 337 | 42.337 | 48.537 | 70.384 | 1.00 | 33.33 | N |
| ATOM | 2429 | CA | ALA | A | 337 | 41.504 | 49.606 | 69.857 | 1.00 | 34.14 | C |
| ATOM | 2430 | CB | ALA | A | 337 | 40.203 | 49.028 | 69.264 | 1.00 | 32.01 | C |
| ATOM | 2431 | C | ALA | A | 337 | 41.189 | 50.582 | 70.990 | 1.00 | 34.28 | C |
| ATOM | 2432 | O | ALA | A | 337 | 41.342 | 51.798 | 70.843 | 1.00 | 33.15 | O |
| ATOM | 2433 | N | LYS | A | 338 | 40.777 | 50.034 | 72.128 | 1.00 | 35.28 | N |
| ATOM | 2434 | CA | LYS | A | 338 | 40.429 | 50.846 | 73.283 | 1.00 | 36.94 | C |
| ATOM | 2435 | CB | LYS | A | 338 | 39.819 | 49.974 | 74.387 | 1.00 | 37.18 | C |
| ATOM | 2436 | CG | LYS | A | 338 | 38.350 | 49.698 | 74.189 | 1.00 | 38.84 | C |
| ATOM | 2437 | CD | LYS | A | 338 | 37.811 | 48.649 | 75.134 | 1.00 | 41.23 | C |
| ATOM | 2438 | CE | LYS | A | 338 | 36.286 | 48.643 | 75.052 | 1.00 | 43.51 | C |
| ATOM | 2439 | NZ | LYS | A | 338 | 35.697 | 47.321 | 75.415 | 1.00 | 48.35 | N |
| ATOM | 2440 | C | LYS | A | 338 | 41.607 | 51.613 | 73.848 | 1.00 | 37.40 | C |
| ATOM | 2441 | O | LYS | A | 338 | 41.445 | 52.729 | 74.339 | 1.00 | 37.91 | O |
| ATOM | 2442 | N | TYR | A | 339 | 42.795 | 51.024 | 73.751 | 1.00 | 37.18 | N |
| ATOM | 2443 | CA | TYR | A | 339 | 44.001 | 51.633 | 74.303 | 1.00 | 37.08 | C |
| ATOM | 2444 | CB | TYR | A | 339 | 45.237 | 50.843 | 73.856 | 1.00 | 35.80 | C |
| ATOM | 2445 | CG | TYR | A | 339 | 46.538 | 51.417 | 74.354 | 1.00 | 33.88 | C |
| ATOM | 2446 | CD1 | TYR | A | 339 | 46.697 | 51.775 | 75.693 | 1.00 | 35.66 | C |
| ATOM | 2447 | CE1 | TYR | A | 339 | 47.901 | 52.303 | 76.172 | 1.00 | 33.13 | C |
| ATOM | 2448 | CD2 | TYR | A | 339 | 47.613 | 51.591 | 73.499 | 1.00 | 34.22 | C |
| ATOM | 2449 | CE2 | TYR | A | 339 | 48.827 | 52.112 | 73.963 | 1.00 | 35.76 | C |
| ATOM | 2450 | CZ | TYR | A | 339 | 48.963 | 52.469 | 75.300 | 1.00 | 36.11 | C |
| ATOM | 2451 | OH | TYR | A | 339 | 50.153 | 53.016 | 75.751 | 1.00 | 37.07 | O |
| ATOM | 2452 | C | TYR | A | 339 | 44.167 | 53.125 | 73.998 | 1.00 | 36.88 | C |
| ATOM | 2453 | O | TYR | A | 339 | 44.625 | 53.888 | 74.855 | 1.00 | 36.77 | O |
| ATOM | 2454 | N | PHE | A | 340 | 43.799 | 53.536 | 72.787 | 1.00 | 36.47 | N |
| ATOM | 2455 | CA | PHE | A | 340 | 43.879 | 54.948 | 72.396 | 1.00 | 36.81 | C |
| ATOM | 2456 | CB | PHE | A | 340 | 44.753 | 55.118 | 71.148 | 1.00 | 37.92 | C |
| ATOM | 2457 | CG | PHE | A | 340 | 46.225 | 55.198 | 71.451 | 1.00 | 39.27 | C |
| ATOM | 2458 | CD1 | PHE | A | 340 | 46.739 | 56.281 | 72.169 | 1.00 | 39.72 | C |
| ATOM | 2459 | CD2 | PHE | A | 340 | 47.091 | 54.182 | 71.054 | 1.00 | 38.81 | C |
| ATOM | 2460 | CE1 | PHE | A | 340 | 48.098 | 56.352 | 72.494 | 1.00 | 40.52 | C |
| ATOM | 2461 | CE2 | PHE | A | 340 | 48.445 | 54.236 | 71.368 | 1.00 | 39.53 | C |
| ATOM | 2462 | CZ | PHE | A | 340 | 48.955 | 55.324 | 72.092 | 1.00 | 40.46 | C |
| ATOM | 2463 | C | PHE | A | 340 | 42.491 | 55.564 | 72.163 | 1.00 | 36.25 | C |
| ATOM | 2464 | O | PHE | A | 340 | 42.286 | 56.339 | 71.219 | 1.00 | 35.38 | O |
| ATOM | 2465 | N | GLN | A | 341 | 41.548 | 55.204 | 73.036 | 1.00 | 35.94 | N |
| ATOM | 2466 | CA | GLN | A | 341 | 40.176 | 55.707 | 72.988 | 1.00 | 36.30 | C |
| ATOM | 2467 | CB | GLN | A | 341 | 40.146 | 57.251 | 73.020 | 1.00 | 36.28 | C |
| ATOM | 2468 | CG | GLN | A | 341 | 40.814 | 57.912 | 74.218 | 1.00 | 36.10 | C |
| ATOM | 2469 | CD | GLN | A | 341 | 40.259 | 57.440 | 75.556 | 1.00 | 38.49 | C |
| ATOM | 2470 | OE1 | GLN | A | 341 | 39.045 | 57.324 | 75.742 | 1.00 | 41.04 | O |
| ATOM | 2471 | NE2 | GLN | A | 341 | 41.154 | 57.182 | 76.501 | 1.00 | 36.74 | N |
| ATOM | 2472 | C | GLN | A | 341 | 39.391 | 55.226 | 71.777 | 1.00 | 35.81 | C |
| ATOM | 2473 | O | GLN | A | 341 | 38.442 | 55.874 | 71.358 | 1.00 | 37.00 | O |
| ATOM | 2474 | N | GLY | A | 342 | 39.787 | 54.101 | 71.211 | 1.00 | 35.36 | N |
| ATOM | 2475 | CA | GLY | A | 342 | 39.067 | 53.568 | 70.073 | 1.00 | 35.07 | C |
| ATOM | 2476 | C | GLY | A | 342 | 38.218 | 52.413 | 70.563 | 1.00 | 35.37 | C |
| ATOM | 2477 | O | GLY | A | 342 | 38.045 | 52.253 | 71.768 | 1.00 | 36.41 | O |
| ATOM | 2478 | N | ASP | A | 343 | 37.691 | 51.609 | 69.646 | 1.00 | 34.07 | N |
| ATOM | 2479 | CA | ASP | A | 343 | 36.875 | 50.456 | 70.011 | 1.00 | 32.74 | C |
| ATOM | 2480 | CB | ASP | A | 343 | 35.422 | 50.878 | 70.311 | 1.00 | 30.26 | C |
| ATOM | 2481 | CG | ASP | A | 343 | 34.660 | 49.851 | 71.162 | 1.00 | 30.47 | C |
| ATOM | 2482 | OD1 | ASP | A | 343 | 35.187 | 48.765 | 71.474 | 1.00 | 31.15 | O |
| ATOM | 2483 | OD2 | ASP | A | 343 | 33.505 | 50.119 | 71.533 | 1.00 | 33.78 | O |
| ATOM | 2484 | C | ASP | A | 343 | 36.904 | 49.473 | 68.839 | 1.00 | 31.67 | C |
| ATOM | 2485 | O | ASP | A | 343 | 37.436 | 49.774 | 67.779 | 1.00 | 31.52 | O |
| ATOM | 2486 | N | LEU | A | 344 | 36.371 | 48.279 | 69.055 | 1.00 | 31.28 | N |
| ATOM | 2487 | CA | LEU | A | 344 | 36.302 | 47.264 | 68.021 | 1.00 | 31.18 | C |
| ATOM | 2488 | CB | LEU | A | 344 | 37.483 | 46.300 | 68.131 | 1.00 | 31.96 | C |
| ATOM | 2489 | CG | LEU | A | 344 | 37.718 | 45.368 | 66.937 | 1.00 | 33.18 | C |
| ATOM | 2490 | CD1 | LEU | A | 344 | 39.127 | 44.784 | 67.028 | 1.00 | 32.85 | C |
| ATOM | 2491 | CD2 | LEU | A | 344 | 36.664 | 44.267 | 66.903 | 1.00 | 31.89 | C |
| ATOM | 2492 | C | LEU | A | 344 | 34.983 | 46.537 | 68.277 | 1.00 | 32.30 | C |
| ATOM | 2493 | O | LEU | A | 344 | 34.818 | 45.876 | 69.296 | 1.00 | 31.99 | O |
| ATOM | 2494 | N | GLN | A | 345 | 34.042 | 46.685 | 67.353 | 1.00 | 32.24 | N |
| ATOM | 2495 | CA | GLN | A | 345 | 32.730 | 46.076 | 67.489 | 1.00 | 31.88 | C |
| ATOM | 2496 | CB | GLN | A | 345 | 31.654 | 47.158 | 67.513 | 1.00 | 31.11 | C |
| ATOM | 2497 | CG | GLN | A | 345 | 31.768 | 48.074 | 68.708 | 1.00 | 31.99 | C |
| ATOM | 2498 | CD | GLN | A | 345 | 30.769 | 49.206 | 68.687 | 1.00 | 32.28 | C |
| ATOM | 2499 | OE1 | GLN | A | 345 | 30.054 | 49.407 | 67.721 | 1.00 | 32.89 | O |
| ATOM | 2500 | NE2 | GLN | A | 345 | 30.738 | 49.969 | 69.758 | 1.00 | 35.15 | N |
| ATOM | 2501 | C | GLN | A | 345 | 32.416 | 45.106 | 66.385 | 1.00 | 33.03 | C |
| ATOM | 2502 | O | GLN | A | 345 | 32.928 | 45.219 | 65.269 | 1.00 | 31.93 | O |
| ATOM | 2503 | N | LEU | A | 346 | 31.552 | 44.152 | 66.710 | 1.00 | 33.16 | N |
| ATOM | 2504 | CA | LEU | A | 346 | 31.125 | 43.149 | 65.754 | 1.00 | 34.36 | C |
| ATOM | 2505 | CB | LEU | A | 346 | 31.634 | 41.773 | 66.176 | 1.00 | 35.30 | C |
| ATOM | 2506 | CG | LEU | A | 346 | 32.543 | 40.995 | 65.224 | 1.00 | 38.96 | C |
| ATOM | 2507 | CD1 | LEU | A | 346 | 33.890 | 41.680 | 65.084 | 1.00 | 39.34 | C |
| ATOM | 2508 | CD2 | LEU | A | 346 | 32.751 | 39.599 | 65.776 | 1.00 | 41.67 | C |
| ATOM | 2509 | C | LEU | A | 346 | 29.604 | 43.083 | 65.657 | 1.00 | 33.70 | C |
| ATOM | 2510 | O | LEU | A | 346 | 28.923 | 43.148 | 66.671 | 1.00 | 34.44 | O |
| ATOM | 2511 | N | PHE | A | 347 | 29.080 | 42.988 | 64.440 | 1.00 | 32.76 | N |
| ATOM | 2512 | CA | PHE | A | 347 | 27.643 | 42.806 | 64.223 | 1.00 | 35.46 | C |
| ATOM | 2513 | CB | PHE | A | 347 | 26.880 | 44.114 | 63.977 | 1.00 | 38.37 | C |
| ATOM | 2514 | CG | PHE | A | 347 | 27.750 | 45.285 | 63.839 | 1.00 | 41.28 | C |
| ATOM | 2515 | CD1 | PHE | A | 347 | 28.148 | 45.714 | 62.594 | 1.00 | 44.84 | C |
| ATOM | 2516 | CD2 | PHE | A | 347 | 28.245 | 45.917 | 64.963 | 1.00 | 44.22 | C |
| ATOM | 2517 | CE1 | PHE | A | 347 | 29.045 | 46.759 | 62.470 | 1.00 | 47.20 | C |
| ATOM | 2518 | CE2 | PHE | A | 347 | 29.137 | 46.956 | 64.855 | 1.00 | 45.26 | C |
| ATOM | 2519 | CZ | PHE | A | 347 | 29.541 | 47.378 | 63.609 | 1.00 | 46.89 | C |
| ATOM | 2520 | C | PHE | A | 347 | 27.470 | 41.872 | 63.042 | 1.00 | 35.05 | C |
| ATOM | 2521 | O | PHE | A | 347 | 28.001 | 42.093 | 61.949 | 1.00 | 35.30 | O |
| ATOM | 2522 | N | SER | A | 348 | 26.708 | 40.820 | 63.278 | 1.00 | 35.91 | N |
| ATOM | 2523 | CA | SER | A | 348 | 26.497 | 39.804 | 62.273 | 1.00 | 36.50 | C |
| ATOM | 2524 | CB | SER | A | 348 | 26.901 | 38.454 | 62.881 | 1.00 | 33.80 | C |
| ATOM | 2525 | OG | SER | A | 348 | 27.324 | 37.566 | 61.889 | 1.00 | 37.53 | O |
| ATOM | 2526 | C | SER | A | 348 | 25.048 | 39.769 | 61.808 | 1.00 | 36.57 | C |
| ATOM | 2527 | O | SER | A | 348 | 24.177 | 40.410 | 62.383 | 1.00 | 35.85 | O |
| ATOM | 2528 | N | MET | A | 349 | 24.819 | 39.008 | 60.748 | 1.00 | 38.95 | N |
| ATOM | 2529 | CA | MET | A | 349 | 23.497 | 38.790 | 60.171 | 1.00 | 39.91 | C |
| ATOM | 2530 | CB | MET | A | 349 | 23.363 | 39.466 | 58.803 | 1.00 | 42.93 | C |
| ATOM | 2531 | CG | MET | A | 349 | 23.632 | 40.957 | 58.748 | 1.00 | 45.51 | C |
| ATOM | 2532 | SD | MET | A | 349 | 22.233 | 41.898 | 59.274 | 1.00 | 51.88 | S |
| ATOM | 2533 | CE | MET | A | 349 | 21.193 | 41.898 | 57.782 | 1.00 | 47.22 | C |
| ATOM | 2534 | C | MET | A | 349 | 23.520 | 37.286 | 59.952 | 1.00 | 39.87 | C |
| ATOM | 2535 | O | MET | A | 349 | 23.974 | 36.838 | 58.905 | 1.00 | 40.76 | O |
| ATOM | 2536 | N | GLU | A | 350 | 23.059 | 36.503 | 60.919 | 1.00 | 39.85 | N |
| ATOM | 2537 | CA | GLU | A | 350 | 23.097 | 35.059 | 60.743 | 1.00 | 41.06 | C |
| ATOM | 2538 | CB | GLU | A | 350 | 22.351 | 34.339 | 61.868 | 1.00 | 42.33 | C |
| ATOM | 2539 | CG | GLU | A | 350 | 22.756 | 32.879 | 61.997 | 1.00 | 43.63 | C |
| ATOM | 2540 | CD | GLU | A | 350 | 21.772 | 32.062 | 62.813 | 1.00 | 44.31 | C |
| ATOM | 2541 | OE1 | GLU | A | 350 | 22.120 | 30.924 | 63.201 | 1.00 | 45.54 | O |
| ATOM | 2542 | OE2 | GLU | A | 350 | 20.650 | 32.549 | 63.065 | 1.00 | 43.54 | O |
| ATOM | 2543 | C | GLU | A | 350 | 22.509 | 34.651 | 59.401 | 1.00 | 39.95 | C |
| ATOM | 2544 | O | GLU | A | 350 | 21.426 | 35.092 | 59.031 | 1.00 | 38.83 | O |
| ATOM | 2545 | N | GLY | A | 351 | 23.251 | 33.813 | 58.681 | 1.00 | 40.39 | N |
| ATOM | 2546 | CA | GLY | A | 351 | 22.813 | 33.336 | 57.380 | 1.00 | 40.58 | C |
| ATOM | 2547 | C | GLY | A | 351 | 23.359 | 34.166 | 56.242 | 1.00 | 40.62 | C |
| ATOM | 2548 | O | GLY | A | 351 | 23.063 | 33.918 | 55.075 | 1.00 | 41.70 | O |
| ATOM | 2549 | N | PHE | A | 352 | 24.183 | 35.148 | 56.564 | 1.00 | 40.74 | N |
| ATOM | 2550 | CA | PHE | A | 352 | 24.711 | 36.001 | 55.522 | 1.00 | 41.79 | C |
| ATOM | 2551 | CB | PHE | A | 352 | 23.856 | 37.260 | 55.405 | 1.00 | 43.28 | C |
| ATOM | 2552 | CG | PHE | A | 352 | 24.320 | 38.189 | 54.334 | 1.00 | 45.36 | C |
| ATOM | 2553 | CD1 | PHE | A | 352 | 24.278 | 37.798 | 52.999 | 1.00 | 47.16 | C |
| ATOM | 2554 | CD2 | PHE | A | 352 | 24.856 | 39.433 | 54.652 | 1.00 | 47.51 | C |
| ATOM | 2555 | CE1 | PHE | A | 352 | 24.768 | 38.631 | 51.995 | 1.00 | 47.56 | C |
| ATOM | 2556 | CE2 | PHE | A | 352 | 25.350 | 40.274 | 53.653 | 1.00 | 47.38 | C |
| ATOM | 2557 | CZ | PHE | A | 352 | 25.306 | 39.870 | 52.324 | 1.00 | 47.45 | C |
| ATOM | 2558 | C | PHE | A | 352 | 26.173 | 36.400 | 55.700 | 1.00 | 41.79 | C |
| ATOM | 2559 | O | PHE | A | 352 | 27.025 | 36.017 | 54.902 | 1.00 | 42.23 | O |
| ATOM | 2560 | N | GLY | A | 353 | 26.457 | 37.183 | 56.736 | 1.00 | 41.03 | N |
| ATOM | 2561 | CA | GLY | A | 353 | 27.820 | 37.610 | 56.968 | 1.00 | 39.65 | C |
| ATOM | 2562 | C | GLY | A | 353 | 28.015 | 38.418 | 58.234 | 1.00 | 38.25 | C |
| ATOM | 2563 | O | GLY | A | 353 | 27.101 | 38.584 | 59.031 | 1.00 | 39.62 | O |
| ATOM | 2564 | N | THR | A | 354 | 29.219 | 38.947 | 58.401 | 1.00 | 36.62 | N |
| ATOM | 2565 | CA | THR | A | 354 | 29.557 | 39.715 | 59.579 | 1.00 | 33.22 | C |
| ATOM | 2566 | CB | THR | A | 354 | 30.436 | 38.886 | 60.539 | 1.00 | 30.98 | C |
| ATOM | 2567 | OG1 | THR | A | 354 | 29.765 | 37.672 | 60.882 | 1.00 | 27.07 | O |
| ATOM | 2568 | CG2 | THR | A | 354 | 30.728 | 39.674 | 61.812 | 1.00 | 28.32 | C |
| ATOM | 2569 | C | THR | A | 354 | 30.325 | 40.989 | 59.262 | 1.00 | 33.93 | C |
| ATOM | 2570 | O | THR | A | 354 | 31.121 | 41.034 | 58.324 | 1.00 | 33.44 | O |
| ATOM | 2571 | N | ASP | A | 355 | 30.075 | 42.025 | 60.055 | 1.00 | 33.79 | N |
| ATOM | 2572 | CA | ASP | A | 355 | 30.797 | 43.283 | 59.913 | 1.00 | 34.83 | C |
| ATOM | 2573 | CB | ASP | A | 355 | 29.843 | 44.469 | 59.745 | 1.00 | 35.66 | C |
| ATOM | 2574 | CG | ASP | A | 355 | 29.261 | 44.550 | 58.344 | 1.00 | 37.57 | C |
| ATOM | 2575 | OD1 | ASP | A | 355 | 29.349 | 43.554 | 57.599 | 1.00 | 38.39 | O |
| ATOM | 2576 | OD2 | ASP | A | 355 | 28.704 | 45.605 | 57.982 | 1.00 | 38.64 | O |
| ATOM | 2577 | C | ASP | A | 355 | 31.617 | 43.466 | 61.179 | 1.00 | 34.21 | C |
| ATOM | 2578 | O | ASP | A | 355 | 31.107 | 43.385 | 62.288 | 1.00 | 34.36 | O |
| ATOM | 2579 | N | ALA | A | 356 | 32.911 | 43.656 | 61.009 | 1.00 | 33.93 | N |
| ATOM | 2580 | CA | ALA | A | 356 | 33.779 | 43.879 | 62.143 | 1.00 | 32.92 | C |
| ATOM | 2581 | CB | ALA | A | 356 | 34.942 | 42.907 | 62.113 | 1.00 | 33.09 | C |
| ATOM | 2582 | C | ALA | A | 356 | 34.250 | 45.313 | 61.934 | 1.00 | 32.41 | C |
| ATOM | 2583 | O | ALA | A | 356 | 34.725 | 45.667 | 60.858 | 1.00 | 32.95 | O |
| ATOM | 2584 | N | VAL | A | 357 | 34.082 | 46.153 | 62.944 | 1.00 | 32.03 | N |
| ATOM | 2585 | CA | VAL | A | 357 | 34.480 | 47.543 | 62.811 | 1.00 | 31.80 | C |
| ATOM | 2586 | CB | VAL | A | 357 | 33.272 | 48.484 | 62.946 | 1.00 | 32.01 | C |
| ATOM | 2587 | CG1 | VAL | A | 357 | 33.722 | 49.932 | 62.715 | 1.00 | 30.93 | C |
| ATOM | 2588 | CG2 | VAL | A | 357 | 32.191 | 48.082 | 61.964 | 1.00 | 32.89 | C |
| ATOM | 2589 | C | VAL | A | 357 | 35.504 | 48.010 | 63.830 | 1.00 | 30.28 | C |
| ATOM | 2590 | O | VAL | A | 357 | 35.329 | 47.811 | 65.027 | 1.00 | 29.66 | O |
| ATOM | 2591 | N | ILE | A | 358 | 36.555 | 48.658 | 63.342 | 1.00 | 29.94 | N |
| ATOM | 2592 | CA | ILE | A | 358 | 37.574 | 49.227 | 64.202 | 1.00 | 31.53 | C |
| ATOM | 2593 | CB | ILE | A | 358 | 38.999 | 48.992 | 63.667 | 1.00 | 32.51 | C |
| ATOM | 2594 | CG2 | ILE | A | 358 | 40.014 | 49.684 | 64.596 | 1.00 | 31.59 | C |
| ATOM | 2595 | CG1 | ILE | A | 358 | 39.290 | 47.490 | 63.578 | 1.00 | 31.66 | C |
| ATOM | 2596 | CD1 | ILE | A | 358 | 40.580 | 47.181 | 62.855 | 1.00 | 33.14 | C |
| ATOM | 2597 | C | ILE | A | 358 | 37.343 | 50.727 | 64.223 | 1.00 | 31.14 | C |
| ATOM | 2598 | O | ILE | A | 358 | 37.347 | 51.366 | 63.177 | 1.00 | 31.44 | O |
| ATOM | 2599 | N | TYR | A | 359 | 37.132 | 51.275 | 65.414 | 1.00 | 32.16 | N |
| ATOM | 2600 | CA | TYR | A | 359 | 36.914 | 52.704 | 65.612 | 1.00 | 31.86 | C |
| ATOM | 2601 | CB | TYR | A | 359 | 35.737 | 52.943 | 66.553 | 1.00 | 32.63 | C |
| ATOM | 2602 | CG | TYR | A | 359 | 34.408 | 52.568 | 65.957 | 1.00 | 33.91 | C |
| ATOM | 2603 | CD1 | TYR | A | 359 | 33.759 | 51.383 | 66.328 | 1.00 | 32.70 | C |
| ATOM | 2604 | CE1 | TYR | A | 359 | 32.535 | 51.026 | 65.754 | 1.00 | 33.88 | C |
| ATOM | 2605 | CD2 | TYR | A | 359 | 33.802 | 53.388 | 64.995 | 1.00 | 33.26 | C |
| ATOM | 2606 | CE2 | TYR | A | 359 | 32.583 | 53.035 | 64.413 | 1.00 | 34.76 | C |
| ATOM | 2607 | CZ | TYR | A | 359 | 31.956 | 51.856 | 64.801 | 1.00 | 34.15 | C |
| ATOM | 2608 | OH | TYR | A | 359 | 30.747 | 51.528 | 64.247 | 1.00 | 36.87 | O |
| ATOM | 2609 | C | TYR | A | 359 | 38.152 | 53.313 | 66.247 | 1.00 | 32.20 | C |
| ATOM | 2610 | O | TYR | A | 359 | 38.513 | 52.952 | 67.361 | 1.00 | 33.46 | O |
| ATOM | 2611 | N | LEU | A | 360 | 38.797 | 54.239 | 65.546 | 1.00 | 33.25 | N |
| ATOM | 2612 | CA | LEU | A | 360 | 39.991 | 54.910 | 66.059 | 1.00 | 33.90 | C |
| ATOM | 2613 | CB | LEU | A | 360 | 41.189 | 54.658 | 65.151 | 1.00 | 31.89 | C |
| ATOM | 2614 | CG | LEU | A | 360 | 41.544 | 53.217 | 64.816 | 1.00 | 32.47 | C |
| ATOM | 2615 | CD1 | LEU | A | 360 | 42.648 | 53.207 | 63.758 | 1.00 | 30.23 | C |
| ATOM | 2616 | CD2 | LEU | A | 360 | 41.994 | 52.481 | 66.102 | 1.00 | 30.87 | C |
| ATOM | 2617 | C | LEU | A | 360 | 39.724 | 56.400 | 66.069 | 1.00 | 33.71 | C |
| ATOM | 2618 | O | LEU | A | 360 | 38.841 | 56.872 | 65.350 | 1.00 | 32.69 | O |
| ATOM | 2619 | N | LYS | A | 361 | 40.486 | 57.137 | 66.878 | 1.00 | 35.04 | N |
| ATOM | 2620 | CA | LYS | A | 361 | 40.345 | 58.591 | 66.945 | 1.00 | 35.26 | C |
| ATOM | 2621 | CB | LYS | A | 361 | 40.943 | 59.124 | 68.240 | 1.00 | 34.79 | C |
| ATOM | 2622 | CG | LYS | A | 361 | 40.155 | 58.783 | 69.487 | 1.00 | 34.60 | C |
| ATOM | 2623 | CD | LYS | A | 361 | 38.750 | 59.378 | 69.462 | 1.00 | 34.41 | C |
| ATOM | 2624 | CE | LYS | A | 361 | 38.123 | 59.306 | 70.863 | 1.00 | 34.82 | C |
| ATOM | 2625 | NZ | LYS | A | 361 | 36.669 | 59.660 | 70.900 | 1.00 | 35.78 | N |
| ATOM | 2626 | C | LYS | A | 361 | 41.101 | 59.165 | 65.765 | 1.00 | 36.76 | C |
| ATOM | 2627 | O | LYS | A | 361 | 42.247 | 58.785 | 65.519 | 1.00 | 37.35 | O |
| ATOM | 2628 | N | ALA | A | 362 | 40.471 | 60.069 | 65.023 | 1.00 | 38.35 | N |
| ATOM | 2629 | CA | ALA | A | 362 | 41.141 | 60.660 | 63.862 | 1.00 | 40.49 | C |
| ATOM | 2630 | CB | ALA | A | 362 | 40.105 | 61.307 | 62.919 | 1.00 | 39.56 | C |
| ATOM | 2631 | C | ALA | A | 362 | 42.225 | 61.688 | 64.233 | 1.00 | 40.92 | C |
| ATOM | 2632 | O | ALA | A | 362 | 43.226 | 61.807 | 63.534 | 1.00 | 41.43 | O |
| ATOM | 2633 | N | LEU | A | 363 | 42.023 | 62.415 | 65.329 | 1.00 | 42.53 | N |
| ATOM | 2634 | CA | LEU | A | 363 | 42.966 | 63.447 | 65.772 | 1.00 | 44.52 | C |
| ATOM | 2635 | CB | LEU | A | 363 | 42.190 | 64.655 | 66.313 | 1.00 | 44.80 | C |
| ATOM | 2636 | CG | LEU | A | 363 | 41.073 | 65.217 | 65.415 | 1.00 | 47.08 | C |
| ATOM | 2637 | CD1 | LEU | A | 363 | 40.310 | 66.330 | 66.138 | 1.00 | 46.26 | C |
| ATOM | 2638 | CD2 | LEU | A | 363 | 41.674 | 65.733 | 64.125 | 1.00 | 47.09 | C |
| ATOM | 2639 | C | LEU | A | 363 | 43.951 | 62.958 | 66.840 | 1.00 | 45.29 | C |
| ATOM | 2640 | O | LEU | A | 363 | 43.559 | 62.306 | 67.809 | 1.00 | 44.60 | O |
| ATOM | 2641 | N | SER | A | 364 | 45.227 | 63.290 | 66.670 | 1.00 | 46.66 | N |
| ATOM | 2642 | CA | SER | A | 364 | 46.243 | 62.873 | 67.632 | 1.00 | 48.43 | C |
| ATOM | 2643 | CB | SER | A | 364 | 47.630 | 63.295 | 67.154 | 1.00 | 48.67 | C |
| ATOM | 2644 | OG | SER | A | 364 | 47.674 | 64.690 | 66.934 | 1.00 | 51.17 | O |
| ATOM | 2645 | C | SER | A | 364 | 45.952 | 63.484 | 69.000 | 1.00 | 48.83 | C |
| ATOM | 2646 | O | SER | A | 364 | 46.425 | 63.005 | 70.026 | 1.00 | 48.53 | O |
| ATOM | 2647 | N | THR | A | 365 | 45.154 | 64.541 | 69.013 | 1.00 | 49.83 | N |
| ATOM | 2648 | CA | THR | A | 365 | 44.795 | 65.187 | 70.262 | 1.00 | 50.70 | C |
| ATOM | 2649 | CB | THR | A | 365 | 44.015 | 66.484 | 70.012 | 1.00 | 52.09 | C |
| ATOM | 2650 | OG1 | THR | A | 365 | 44.665 | 67.244 | 68.992 | 1.00 | 53.61 | O |
| ATOM | 2651 | CG2 | THR | A | 365 | 43.949 | 67.313 | 71.284 | 1.00 | 52.67 | C |
| ATOM | 2652 | C | THR | A | 365 | 43.898 | 64.282 | 71.097 | 1.00 | 50.91 | C |
| ATOM | 2653 | O | THR | A | 365 | 43.971 | 64.274 | 72.327 | 1.00 | 50.99 | O |
| ATOM | 2654 | N | ASP | A | 366 | 43.040 | 63.525 | 70.418 | 1.00 | 50.56 | N |
| ATOM | 2655 | CA | ASP | A | 366 | 42.097 | 62.642 | 71.098 | 1.00 | 50.46 | C |
| ATOM | 2656 | CB | ASP | A | 366 | 40.789 | 62.537 | 70.293 | 1.00 | 50.03 | C |
| ATOM | 2657 | CG | ASP | A | 366 | 40.124 | 63.891 | 70.055 | 1.00 | 49.95 | C |
| ATOM | 2658 | OD1 | ASP | A | 366 | 39.980 | 64.661 | 71.016 | 1.00 | 50.97 | O |
| ATOM | 2659 | OD2 | ASP | A | 366 | 39.728 | 64.187 | 68.908 | 1.00 | 50.92 | O |
| ATOM | 2660 | C | ASP | A | 366 | 42.648 | 61.240 | 71.350 | 1.00 | 49.95 | C |
| ATOM | 2661 | O | ASP | A | 366 | 42.010 | 60.432 | 72.022 | 1.00 | 49.46 | O |
| ATOM | 2662 | N | SER | A | 367 | 43.826 | 60.954 | 70.808 | 1.00 | 49.33 | N |
| ATOM | 2663 | CA | SER | A | 367 | 44.438 | 59.642 | 70.979 | 1.00 | 49.67 | C |
| ATOM | 2664 | CB | SER | A | 367 | 45.329 | 59.310 | 69.782 | 1.00 | 48.95 | C |
| ATOM | 2665 | OG | SER | A | 367 | 44.566 | 59.308 | 68.596 | 1.00 | 49.56 | O |
| ATOM | 2666 | C | SER | A | 367 | 45.252 | 59.618 | 72.258 | 1.00 | 49.43 | C |
| ATOM | 2667 | O | SER | A | 367 | 46.472 | 59.735 | 72.234 | 1.00 | 50.18 | O |
| ATOM | 2668 | N | VAL | A | 368 | 44.554 | 59.472 | 73.375 | 1.00 | 48.91 | N |
| ATOM | 2669 | CA | VAL | A | 368 | 45.188 | 59.436 | 74.678 | 1.00 | 48.75 | C |
| ATOM | 2670 | CB | VAL | A | 368 | 44.547 | 60.458 | 75.633 | 1.00 | 48.61 | C |
| ATOM | 2671 | CG1 | VAL | A | 368 | 45.301 | 60.485 | 76.944 | 1.00 | 48.38 | C |
| ATOM | 2672 | CG2 | VAL | A | 368 | 44.539 | 61.824 | 74.990 | 1.00 | 48.90 | C |
| ATOM | 2673 | C | VAL | A | 368 | 45.032 | 58.052 | 75.284 | 1.00 | 48.76 | C |
| ATOM | 2674 | O | VAL | A | 368 | 43.967 | 57.432 | 75.188 | 1.00 | 49.04 | O |
| ATOM | 2675 | N | GLU | A | 369 | 46.096 | 57.581 | 75.916 | 1.00 | 48.16 | N |
| ATOM | 2676 | CA | GLU | A | 369 | 46.100 | 56.273 | 76.547 | 1.00 | 48.29 | C |
| ATOM | 2677 | CB | GLU | A | 369 | 47.336 | 56.100 | 77.422 | 1.00 | 46.83 | C |
| ATOM | 2678 | CG | GLU | A | 369 | 48.648 | 55.963 | 76.703 | 1.00 | 44.80 | C |
| ATOM | 2679 | CD | GLU | A | 369 | 49.754 | 55.661 | 77.687 | 1.00 | 45.27 | C |
| ATOM | 2680 | OE1 | GLU | A | 369 | 50.070 | 56.558 | 78.513 | 1.00 | 43.82 | O |
| ATOM | 2681 | OE2 | GLU | A | 369 | 50.283 | 54.520 | 77.647 | 1.00 | 42.25 | O |
| ATOM | 2682 | C | GLU | A | 369 | 44.894 | 56.050 | 77.435 | 1.00 | 49.39 | C |
| ATOM | 2683 | O | GLU | A | 369 | 44.493 | 56.936 | 78.186 | 1.00 | 48.50 | O |
| ATOM | 2684 | N | ARG | A | 370 | 44.318 | 54.861 | 77.343 | 1.00 | 50.83 | N |
| ATOM | 2685 | CA | ARG | A | 370 | 43.211 | 54.517 | 78.203 | 1.00 | 54.37 | C |
| ATOM | 2686 | CB | ARG | A | 370 | 42.073 | 53.883 | 77.415 | 1.00 | 54.48 | C |
| ATOM | 2687 | CG | ARG | A | 370 | 40.957 | 53.414 | 78.306 | 1.00 | 56.52 | C |
| ATOM | 2688 | CD | ARG | A | 370 | 39.670 | 53.330 | 77.549 | 1.00 | 59.26 | C |
| ATOM | 2689 | NE | ARG | A | 370 | 38.616 | 52.747 | 78.365 | 1.00 | 62.03 | N |
| ATOM | 2690 | CZ | ARG | A | 370 | 37.490 | 52.252 | 77.867 | 1.00 | 63.68 | C |
| ATOM | 2691 | NH1 | ARG | A | 370 | 37.284 | 52.280 | 76.555 | 1.00 | 63.84 | N |
| ATOM | 2692 | NH2 | ARG | A | 370 | 36.582 | 51.713 | 78.674 | 1.00 | 63.82 | N |
| ATOM | 2693 | C | ARG | A | 370 | 43.833 | 53.516 | 79.175 | 1.00 | 56.76 | C |
| ATOM | 2694 | O | ARG | A | 370 | 44.236 | 52.424 | 78.783 | 1.00 | 57.09 | O |
| ATOM | 2695 | N | LEU | A | 371 | 43.931 | 53.910 | 80.437 | 1.00 | 60.13 | N |
| ATOM | 2696 | CA | LEU | A | 371 | 44.552 | 53.082 | 81.453 | 1.00 | 64.02 | C |
| ATOM | 2697 | CB | LEU | A | 371 | 45.755 | 53.815 | 82.051 | 1.00 | 61.60 | C |
| ATOM | 2698 | CG | LEU | A | 371 | 46.796 | 54.399 | 81.102 | 1.00 | 61.53 | C |
| ATOM | 2699 | CD1 | LEU | A | 371 | 47.826 | 55.208 | 81.889 | 1.00 | 61.76 | C |
| ATOM | 2700 | CD2 | LEU | A | 371 | 47.463 | 53.281 | 80.334 | 1.00 | 59.93 | C |
| ATOM | 2701 | C | LEU | A | 371 | 43.620 | 52.705 | 82.589 | 1.00 | 68.35 | C |
| ATOM | 2702 | O | LEU | A | 371 | 42.781 | 53.490 | 83.001 | 1.00 | 68.98 | O |
| ATOM | 2703 | N | PRO | A | 372 | 43.751 | 51.481 | 83.106 | 1.00 | 73.57 | N |
| ATOM | 2704 | CD | PRO | A | 372 | 44.482 | 50.346 | 82.510 | 1.00 | 74.44 | C |
| ATOM | 2705 | CA | PRO | A | 372 | 42.908 | 51.036 | 84.218 | 1.00 | 78.32 | C |
| ATOM | 2706 | CB | PRO | A | 372 | 42.866 | 49.528 | 84.019 | 1.00 | 77.28 | C |
| ATOM | 2707 | CG | PRO | A | 372 | 44.256 | 49.240 | 83.522 | 1.00 | 75.81 | C |
| ATOM | 2708 | C | PRO | A | 372 | 43.627 | 51.433 | 85.513 | 1.00 | 83.11 | C |
| ATOM | 2709 | O | PRO | A | 372 | 44.844 | 51.267 | 85.614 | 1.00 | 83.43 | O |
| ATOM | 2710 | N | VAL | A | 373 | 42.900 | 51.959 | 86.496 | 1.00 | 89.16 | N |
| ATOM | 2711 | CA | VAL | A | 373 | 43.535 | 52.368 | 87.754 | 1.00 | 95.31 | C |
| ATOM | 2712 | CB | VAL | A | 373 | 43.476 | 53.906 | 87.939 | 1.00 | 95.44 | C |
| ATOM | 2713 | CG1 | VAL | A | 373 | 43.963 | 54.295 | 89.334 | 1.00 | 96.02 | C |
| ATOM | 2714 | CG2 | VAL | A | 373 | 44.339 | 54.580 | 86.892 | 1.00 | 95.91 | C |
| ATOM | 2715 | C | VAL | A | 373 | 42.944 | 51.722 | 88.999 | 1.00 | 99.56 | C |
| ATOM | 2716 | O | VAL | A | 373 | 41.747 | 51.431 | 89.050 | 1.00 | 99.54 | O |
| ATOM | 2717 | N | TYR | A | 374 | 43.795 | 51.506 | 90.002 | 1.00 | 105.05 | N |
| ATOM | 2718 | CA | TYR | A | 374 | 43.366 | 50.911 | 91.266 | 1.00 | 110.53 | C |
| ATOM | 2719 | CB | TYR | A | 374 | 44.535 | 50.199 | 91.968 | 1.00 | 111.51 | C |
| ATOM | 2720 | CG | TYR | A | 374 | 44.173 | 49.674 | 93.345 | 1.00 | 112.99 | C |
| ATOM | 2721 | CD1 | TYR | A | 374 | 43.119 | 48.773 | 93.511 | 1.00 | 113.56 | C |
| ATOM | 2722 | CE1 | TYR | A | 374 | 42.749 | 48.313 | 94.779 | 1.00 | 114.22 | C |
| ATOM | 2723 | CD2 | TYR | A | 374 | 44.856 | 50.105 | 94.486 | 1.00 | 113.45 | C |
| ATOM | 2724 | CE2 | TYR | A | 374 | 44.494 | 49.650 | 95.761 | 1.00 | 114.22 | C |
| ATOM | 2725 | CZ | TYR | A | 374 | 43.440 | 48.755 | 95.898 | 1.00 | 114.47 | C |
| ATOM | 2726 | OH | TYR | A | 374 | 43.078 | 48.296 | 97.148 | 1.00 | 114.77 | O |
| ATOM | 2727 | C | TYR | A | 374 | 42.783 | 51.965 | 92.211 | 1.00 | 113.68 | C |
| ATOM | 2728 | O | TYR | A | 374 | 43.475 | 52.895 | 92.632 | 1.00 | 113.86 | O |
| ATOM | 2729 | N | ASN | A | 375 | 41.505 | 51.808 | 92.538 | 1.00 | 117.71 | N |
| ATOM | 2730 | CA | ASN | A | 375 | 40.821 | 52.725 | 93.443 | 1.00 | 121.83 | C |
| ATOM | 2731 | CB | ASN | A | 375 | 40.111 | 53.835 | 92.655 | 1.00 | 121.76 | C |
| ATOM | 2732 | CG | ASN | A | 375 | 41.069 | 54.686 | 91.847 | 1.00 | 122.19 | C |
| ATOM | 2733 | OD1 | ASN | A | 375 | 41.982 | 55.306 | 92.394 | 1.00 | 122.58 | O |
| ATOM | 2734 | ND2 | ASN | A | 375 | 40.861 | 54.726 | 90.536 | 1.00 | 122.10 | N |
| ATOM | 2735 | C | ASN | A | 375 | 39.787 | 51.938 | 94.236 | 1.00 | 124.69 | C |
| ATOM | 2736 | O | ASN | A | 375 | 39.672 | 50.718 | 94.081 | 1.00 | 125.00 | O |
| ATOM | 2737 | N | LYS | A | 376 | 39.049 | 52.635 | 95.096 | 1.00 | 128.02 | N |
| ATOM | 2738 | CA | LYS | A | 376 | 37.994 | 52.002 | 95.880 | 1.00 | 131.27 | C |
| ATOM | 2739 | CB | LYS | A | 376 | 37.425 | 52.987 | 96.913 | 1.00 | 131.35 | C |
| ATOM | 2740 | CG | LYS | A | 376 | 36.006 | 52.666 | 97.389 | 1.00 | 131.73 | C |
| ATOM | 2741 | CD | LYS | A | 376 | 35.902 | 51.269 | 97.990 | 1.00 | 132.07 | C |
| ATOM | 2742 | CE | LYS | A | 376 | 34.450 | 50.867 | 98.232 | 1.00 | 132.16 | C |
| ATOM | 2743 | NZ | LYS | A | 376 | 33.763 | 51.769 | 99.198 | 1.00 | 132.19 | N |
| ATOM | 2744 | C | LYS | A | 376 | 36.914 | 51.610 | 94.877 | 1.00 | 133.42 | C |
| ATOM | 2745 | O | LYS | A | 376 | 36.093 | 50.726 | 95.129 | 1.00 | 133.79 | O |
| ATOM | 2746 | N | SER | A | 377 | 36.940 | 52.282 | 93.730 | 1.00 | 135.94 | N |
| ATOM | 2747 | CA | SER | A | 377 | 35.990 | 52.041 | 92.650 | 1.00 | 138.44 | C |
| ATOM | 2748 | CB | SER | A | 377 | 36.100 | 53.157 | 91.606 | 1.00 | 138.44 | C |
| ATOM | 2749 | OG | SER | A | 377 | 35.268 | 52.900 | 90.488 | 1.00 | 138.61 | O |
| ATOM | 2750 | C | SER | A | 377 | 36.238 | 50.686 | 91.985 | 1.00 | 140.08 | C |
| ATOM | 2751 | O | SER | A | 377 | 35.305 | 49.907 | 91.782 | 1.00 | 140.35 | O |
| ATOM | 2752 | N | ALA | A | 378 | 37.499 | 50.413 | 91.651 | 1.00 | 141.87 | N |
| ATOM | 2753 | CA | ALA | A | 378 | 37.876 | 49.156 | 91.011 | 1.00 | 143.64 | C |
| ATOM | 2754 | CB | ALA | A | 378 | 39.310 | 49.247 | 90.497 | 1.00 | 143.37 | C |
| ATOM | 2755 | C | ALA | A | 378 | 37.740 | 47.975 | 91.971 | 1.00 | 144.99 | C |
| ATOM | 2756 | O | ALA | A | 378 | 37.620 | 46.827 | 91.543 | 1.00 | 145.12 | O |
| ATOM | 2757 | N | TRP | A | 379 | 37.755 | 48.265 | 93.269 | 1.00 | 146.63 | N |
| ATOM | 2758 | CA | TRP | A | 379 | 37.640 | 47.233 | 94.296 | 1.00 | 148.29 | C |
| ATOM | 2759 | CB | TRP | A | 379 | 37.881 | 47.840 | 95.683 | 1.00 | 148.93 | C |
| ATOM | 2760 | CG | TRP | A | 379 | 37.844 | 46.828 | 96.795 | 1.00 | 149.95 | C |
| ATOM | 2761 | CD2 | TRP | A | 379 | 36.712 | 46.485 | 97.611 | 1.00 | 150.51 | C |
| ATOM | 2762 | CE2 | TRP | A | 379 | 37.128 | 45.459 | 98.491 | 1.00 | 150.72 | C |
| ATOM | 2763 | CE3 | TRP | A | 379 | 35.387 | 46.944 | 97.684 | 1.00 | 150.69 | C |
| ATOM | 2764 | CD1 | TRP | A | 379 | 38.868 | 46.021 | 97.204 | 1.00 | 150.22 | C |
| ATOM | 2765 | NE1 | TRP | A | 379 | 38.445 | 45.197 | 98.221 | 1.00 | 150.58 | N |
| ATOM | 2766 | CZ2 | TRP | A | 379 | 36.264 | 44.882 | 99.435 | 1.00 | 150.91 | C |
| ATOM | 2767 | CZ3 | TRP | A | 379 | 34.528 | 46.370 | 98.623 | 1.00 | 150.83 | C |
| ATOM | 2768 | CH2 | TRP | A | 379 | 34.972 | 45.349 | 99.484 | 1.00 | 150.93 | C |
| ATOM | 2769 | C | TRP | A | 379 | 36.283 | 46.521 | 94.298 | 1.00 | 149.13 | C |
| ATOM | 2770 | O | TRP | A | 379 | 36.224 | 45.291 | 94.272 | 1.00 | 149.23 | O |
| ATOM | 2771 | N | ARG | A | 380 | 35.200 | 47.298 | 94.325 | 1.00 | 150.09 | N |
| ATOM | 2772 | CA | ARG | A | 380 | 33.844 | 46.750 | 94.365 | 1.00 | 150.98 | C |
| ATOM | 2773 | CB | ARG | A | 380 | 32.859 | 47.836 | 94.829 | 1.00 | 150.82 | C |
| ATOM | 2774 | CG | ARG | A | 380 | 32.988 | 49.165 | 94.102 | 1.00 | 150.73 | C |
| ATOM | 2775 | CD | ARG | A | 380 | 32.288 | 50.277 | 94.870 | 1.00 | 150.60 | C |
| ATOM | 2776 | NE | ARG | A | 380 | 32.530 | 51.592 | 94.280 | 1.00 | 150.46 | N |
| ATOM | 2777 | CZ | ARG | A | 380 | 32.224 | 52.745 | 94.868 | 1.00 | 150.32 | C |
| ATOM | 2778 | NH1 | ARG | A | 380 | 31.659 | 52.750 | 96.068 | 1.00 | 150.27 | N |
| ATOM | 2779 | NH2 | ARG | A | 380 | 32.484 | 53.893 | 94.258 | 1.00 | 150.17 | N |
| ATOM | 2780 | C | ARG | A | 380 | 33.310 | 46.067 | 93.098 | 1.00 | 151.61 | C |
| ATOM | 2781 | O | ARG | A | 380 | 32.157 | 45.629 | 93.071 | 1.00 | 151.70 | O |
| ATOM | 2782 | N | HIS | A | 381 | 34.134 | 45.972 | 92.056 | 1.00 | 152.31 | N |
| ATOM | 2783 | CA | HIS | A | 381 | 33.714 | 45.303 | 90.823 | 1.00 | 153.00 | C |
| ATOM | 2784 | CB | HIS | A | 381 | 34.480 | 45.841 | 89.609 | 1.00 | 152.95 | C |
| ATOM | 2785 | CG | HIS | A | 381 | 33.997 | 47.174 | 89.125 | 1.00 | 152.97 | C |
| ATOM | 2786 | CD2 | HIS | A | 381 | 33.396 | 47.539 | 87.968 | 1.00 | 152.92 | C |
| ATOM | 2787 | ND1 | HIS | A | 381 | 34.115 | 48.325 | 89.874 | 1.00 | 152.99 | N |
| ATOM | 2788 | CE1 | HIS | A | 381 | 33.608 | 49.341 | 89.198 | 1.00 | 152.97 | C |
| ATOM | 2789 | NE2 | HIS | A | 381 | 33.165 | 48.891 | 88.038 | 1.00 | 152.92 | N |
| ATOM | 2790 | C | HIS | A | 381 | 33.986 | 43.810 | 90.969 | 1.00 | 153.49 | C |
| ATOM | 2791 | O | HIS | A | 381 | 33.579 | 43.002 | 90.129 | 1.00 | 153.70 | O |
| ATOM | 2792 | N | TYR | A | 382 | 34.682 | 43.457 | 92.046 | 1.00 | 153.92 | N |
| ATOM | 2793 | CA | TYR | A | 382 | 35.027 | 42.073 | 92.341 | 1.00 | 154.28 | C |
| ATOM | 2794 | CB | TYR | A | 382 | 36.499 | 41.976 | 92.748 | 1.00 | 154.21 | C |
| ATOM | 2795 | CG | TYR | A | 382 | 37.475 | 42.337 | 91.650 | 1.00 | 154.19 | C |
| ATOM | 2796 | CD1 | TYR | A | 382 | 37.620 | 41.527 | 90.523 | 1.00 | 154.13 | C |
| ATOM | 2797 | CE1 | TYR | A | 382 | 38.541 | 41.841 | 89.523 | 1.00 | 153.96 | C |
| ATOM | 2798 | CD2 | TYR | A | 382 | 38.273 | 43.477 | 91.750 | 1.00 | 154.15 | C |
| ATOM | 2799 | CE2 | TYR | A | 382 | 39.196 | 43.801 | 90.755 | 1.00 | 154.09 | C |
| ATOM | 2800 | CZ | TYR | A | 382 | 39.325 | 42.977 | 89.647 | 1.00 | 153.98 | C |
| ATOM | 2801 | OH | TYR | A | 382 | 40.240 | 43.284 | 88.666 | 1.00 | 153.66 | O |
| ATOM | 2802 | C | TYR | A | 382 | 34.154 | 41.542 | 93.473 | 1.00 | 154.60 | C |
| ATOM | 2803 | O | TYR | A | 382 | 34.346 | 40.420 | 93.945 | 1.00 | 154.62 | O |
| ATOM | 2804 | N | GLN | A | 383 | 33.199 | 42.359 | 93.909 | 1.00 | 154.99 | N |
| ATOM | 2805 | CA | GLN | A | 383 | 32.290 | 41.972 | 94.984 | 1.00 | 155.32 | C |
| ATOM | 2806 | CB | GLN | A | 383 | 31.969 | 43.174 | 95.882 | 1.00 | 155.17 | C |
| ATOM | 2807 | CG | GLN | A | 383 | 33.180 | 43.868 | 96.491 | 1.00 | 155.07 | C |
| ATOM | 2808 | CD | GLN | A | 383 | 34.200 | 42.893 | 97.046 | 1.00 | 155.12 | C |
| ATOM | 2809 | OE1 | GLN | A | 383 | 35.090 | 42.434 | 96.328 | 1.00 | 155.16 | O |
| ATOM | 2810 | NE2 | GLN | A | 383 | 34.069 | 42.562 | 98.325 | 1.00 | 155.00 | N |
| ATOM | 2811 | C | GLN | A | 383 | 30.992 | 41.420 | 94.403 | 1.00 | 155.58 | C |
| ATOM | 2812 | O | GLN | A | 383 | 29.944 | 41.471 | 95.049 | 1.00 | 155.67 | O |
| ATOM | 2813 | N | THR | A | 384 | 31.066 | 40.895 | 93.182 | 1.00 | 155.78 | N |
| ATOM | 2814 | CA | THR | A | 384 | 29.891 | 40.337 | 92.519 | 1.00 | 155.95 | C |
| ATOM | 2815 | CB | THR | A | 384 | 30.213 | 39.926 | 91.058 | 1.00 | 155.75 | C |
| ATOM | 2816 | OG1 | THR | A | 384 | 30.726 | 41.059 | 90.343 | 1.00 | 155.46 | O |
| ATOM | 2817 | CG2 | THR | A | 384 | 28.957 | 39.433 | 90.353 | 1.00 | 155.60 | C |
| ATOM | 2818 | C | THR | A | 384 | 29.378 | 39.123 | 93.297 | 1.00 | 156.26 | C |
| ATOM | 2819 | O | THR | A | 384 | 30.029 | 38.077 | 93.346 | 1.00 | 156.22 | O |
| ATOM | 2820 | N | ILE | A | 385 | 28.207 | 39.282 | 93.911 | 1.00 | 156.61 | N |
| ATOM | 2821 | CA | ILE | A | 385 | 27.580 | 38.229 | 94.707 | 1.00 | 156.89 | C |
| ATOM | 2822 | CB | ILE | A | 385 | 27.232 | 38.759 | 96.127 | 1.00 | 156.74 | C |
| ATOM | 2823 | CG2 | ILE | A | 385 | 26.726 | 37.626 | 97.010 | 1.00 | 156.77 | C |
| ATOM | 2824 | CG1 | ILE | A | 385 | 28.475 | 39.388 | 96.767 | 1.00 | 156.53 | C |
| ATOM | 2825 | CD1 | ILE | A | 385 | 28.223 | 40.027 | 98.122 | 1.00 | 156.18 | C |
| ATOM | 2826 | C | ILE | A | 385 | 26.304 | 37.743 | 94.011 | 1.00 | 157.23 | C |
| ATOM | 2827 | O | ILE | A | 385 | 25.345 | 37.320 | 94.659 | 1.00 | 157.21 | O |
| ATOM | 2828 | N | ALA | A | 386 | 26.309 | 37.802 | 92.683 | 1.00 | 157.65 | N |
| ATOM | 2829 | CA | ALA | A | 386 | 25.163 | 37.390 | 91.880 | 1.00 | 158.17 | C |
| ATOM | 2830 | CB | ALA | A | 386 | 25.539 | 37.395 | 90.401 | 1.00 | 158.10 | C |
| ATOM | 2831 | C | ALA | A | 386 | 24.568 | 36.035 | 92.265 | 1.00 | 158.54 | C |
| ATOM | 2832 | O | ALA | A | 386 | 25.283 | 35.067 | 92.532 | 1.00 | 158.47 | O |
| ATOM | 2833 | N | ALA | A | 387 | 23.240 | 35.996 | 92.282 | 1.00 | 159.03 | N |
| ATOM | 2834 | CA | ALA | A | 387 | 22.456 | 34.813 | 92.616 | 1.00 | 159.52 | C |
| ATOM | 2835 | CB | ALA | A | 387 | 22.751 | 34.364 | 94.047 | 1.00 | 159.48 | C |
| ATOM | 2836 | C | ALA | A | 387 | 21.000 | 35.257 | 92.482 | 1.00 | 159.86 | C |
| ATOM | 2837 | O | ALA | A | 387 | 20.444 | 35.864 | 93.400 | 1.00 | 159.91 | O |
| ATOM | 2838 | N | ALA | A | 388 | 20.402 | 34.964 | 91.326 | 1.00 | 160.25 | N |
| ATOM | 2839 | CA | ALA | A | 388 | 19.022 | 35.345 | 91.007 | 1.00 | 160.55 | C |
| ATOM | 2840 | CB | ALA | A | 388 | 18.103 | 35.124 | 92.213 | 1.00 | 160.52 | C |
| ATOM | 2841 | C | ALA | A | 388 | 19.006 | 36.817 | 90.584 | 1.00 | 160.79 | C |
| ATOM | 2842 | O | ALA | A | 388 | 19.134 | 37.715 | 91.419 | 1.00 | 160.82 | O |
| ATOM | 2843 | N | GLY | A | 389 | 18.844 | 37.053 | 89.284 | 1.00 | 161.07 | N |
| ATOM | 2844 | CA | GLY | A | 389 | 18.849 | 38.410 | 88.757 | 1.00 | 161.40 | C |
| ATOM | 2845 | C | GLY | A | 389 | 17.595 | 39.260 | 88.895 | 1.00 | 161.56 | C |
| ATOM | 2846 | O | GLY | A | 389 | 16.693 | 38.949 | 89.674 | 1.00 | 161.66 | O |
| ATOM | 2847 | N | ASP | A | 390 | 17.556 | 40.348 | 88.126 | 1.00 | 161.64 | N |
| ATOM | 2848 | CA | ASP | A | 390 | 16.437 | 41.290 | 88.127 | 1.00 | 161.61 | C |
| ATOM | 2849 | CB | ASP | A | 390 | 16.969 | 42.735 | 88.056 | 1.00 | 161.63 | C |
| ATOM | 2850 | CG | ASP | A | 390 | 18.225 | 42.872 | 87.191 | 1.00 | 161.62 | C |
| ATOM | 2851 | OD1 | ASP | A | 390 | 18.663 | 41.871 | 86.585 | 1.00 | 161.57 | O |
| ATOM | 2852 | OD2 | ASP | A | 390 | 18.780 | 43.991 | 87.119 | 1.00 | 161.48 | O |
| ATOM | 2853 | C | ASP | A | 390 | 15.419 | 41.048 | 87.003 | 1.00 | 161.51 | C |
| ATOM | 2854 | O | ASP | A | 390 | 14.391 | 41.726 | 86.926 | 1.00 | 161.30 | O |
| ATOM | 2855 | N | TRP | A | 391 | 15.708 | 40.074 | 86.144 | 1.00 | 161.45 | N |
| ATOM | 2856 | CA | TRP | A | 391 | 14.817 | 39.739 | 85.035 | 1.00 | 161.40 | C |
| ATOM | 2857 | CB | TRP | A | 391 | 15.516 | 39.985 | 83.688 | 1.00 | 161.31 | C |
| ATOM | 2858 | CG | TRP | A | 391 | 16.156 | 41.349 | 83.549 | 1.00 | 161.17 | C |
| ATOM | 2859 | CD2 | TRP | A | 391 | 15.662 | 42.463 | 82.787 | 1.00 | 160.97 | C |
| ATOM | 2860 | CE2 | TRP | A | 391 | 16.575 | 43.528 | 82.963 | 1.00 | 160.85 | C |
| ATOM | 2861 | CE3 | TRP | A | 391 | 14.536 | 42.666 | 81.978 | 1.00 | 160.78 | C |
| ATOM | 2862 | CD1 | TRP | A | 391 | 17.314 | 41.773 | 84.133 | 1.00 | 161.12 | C |
| ATOM | 2863 | NE1 | TRP | A | 391 | 17.574 | 43.079 | 83.786 | 1.00 | 160.98 | N |
| ATOM | 2864 | CZ2 | TRP | A | 391 | 16.399 | 44.777 | 82.357 | 1.00 | 160.63 | C |
| ATOM | 2865 | CZ3 | TRP | A | 391 | 14.361 | 43.910 | 81.374 | 1.00 | 160.64 | C |
| ATOM | 2866 | CH2 | TRP | A | 391 | 15.289 | 44.949 | 81.570 | 1.00 | 160.51 | C |
| ATOM | 2867 | C | TRP | A | 391 | 14.378 | 38.274 | 85.124 | 1.00 | 161.41 | C |
| ATOM | 2868 | O | TRP | A | 391 | 15.147 | 37.409 | 85.551 | 1.00 | 161.43 | O |
| ATOM | 2869 | N | CYS | A | 392 | 13.138 | 38.004 | 84.720 | 1.00 | 161.42 | N |
| ATOM | 2870 | CA | CYS | A | 392 | 12.585 | 36.652 | 84.747 | 1.00 | 161.36 | C |
| ATOM | 2871 | CB | CYS | A | 392 | 11.291 | 36.616 | 85.566 | 1.00 | 161.18 | C |
| ATOM | 2872 | SG | CYS | A | 392 | 9.864 | 37.353 | 84.729 | 1.00 | 160.67 | S |
| ATOM | 2873 | C | CYS | A | 392 | 12.285 | 36.161 | 83.332 | 1.00 | 161.39 | C |
| ATOM | 2874 | O | CYS | A | 392 | 12.589 | 36.898 | 82.371 | 1.00 | 161.36 | O |
| ATOM | 2875 | OXT | CYS | A | 392 | 11.743 | 35.042 | 83.205 | 1.00 | 161.49 | O |
| TER | 2876 | | CYS | A | 392 | | | | | | |
| ATOM | 2877 | O | HOH | W | 1 | 36.010 | 38.832 | 63.526 | 1.00 | 26.33 | O |
| ATOM | 2878 | O | HOH | W | 2 | 36.178 | 39.582 | 60.694 | 1.00 | 31.48 | O |
| ATOM | 2879 | O | HOH | W | 3 | 34.452 | 40.564 | 58.900 | 1.00 | 33.75 | O |
| ATOM | 2880 | O | HOH | W | 4 | 30.551 | 35.935 | 58.879 | 1.00 | 34.80 | O |
| ATOM | 2881 | O | HOH | W | 5 | 31.362 | 30.898 | 64.093 | 1.00 | 58.66 | O |
| ATOM | 2882 | O | HOH | W | 6 | 34.030 | 28.070 | 65.576 | 1.00 | 57.91 | O |
| ATOM | 2883 | O | HOH | W | 7 | 38.813 | 31.799 | 63.159 | 1.00 | 48.90 | O |
| ATOM | 2884 | O | HOH | W | 8 | 41.751 | 33.799 | 63.783 | 1.00 | 47.77 | O |
| ATOM | 2885 | O | HOH | W | 9 | 41.724 | 36.389 | 64.938 | 1.00 | 45.80 | O |
| ATOM | 2886 | O | HOH | W | 10 | 52.999 | 40.557 | 81.494 | 1.00 | 33.02 | O |
| ATOM | 2887 | O | HOH | W | 11 | 46.487 | 50.798 | 69.765 | 1.00 | 32.61 | O |
| ATOM | 2888 | O | HOH | W | 12 | 30.241 | 55.688 | 65.048 | 1.00 | 27.06 | O |
| ATOM | 2889 | O | HOH | W | 13 | 32.499 | 56.271 | 63.349 | 1.00 | 32.90 | O |
| ATOM | 2890 | O | HOH | W | 14 | 39.862 | 62.158 | 66.823 | 1.00 | 42.01 | O |
| ATOM | 2891 | O | HOH | W | 15 | 29.030 | 53.294 | 65.824 | 1.00 | 34.06 | O |
| ATOM | 2892 | O | HOH | W | 16 | 44.034 | 57.195 | 67.497 | 1.00 | 34.72 | O |
| ATOM | 2893 | O | HOH | W | 17 | 36.621 | 56.887 | 67.778 | 1.00 | 49.01 | O |
| ATOM | 2894 | O | HOH | W | 18 | 43.360 | 52.947 | 69.416 | 1.00 | 37.30 | O |
| ATOM | 2895 | O | HOH | W | 19 | 37.976 | 21.808 | 93.272 | 1.00 | 40.93 | O |
| ATOM | 2896 | O | HOH | W | 20 | 55.620 | 43.832 | 80.583 | 1.00 | 58.46 | O |
| ATOM | 2897 | O | HOH | W | 21 | 33.925 | 59.305 | 68.549 | 1.00 | 36.16 | O |
| ATOM | 2898 | O | HOH | W | 22 | 34.637 | 57.601 | 60.668 | 1.00 | 35.42 | O |
| ATOM | 2899 | O | HOH | W | 23 | 43.037 | 33.510 | 85.438 | 1.00 | 51.54 | O |
| ATOM | 2900 | O | HOH | W | 24 | 30.432 | 44.000 | 69.541 | 1.00 | 50.10 | O |
| ATOM | 2901 | O | HOH | W | 25 | 32.380 | 63.758 | 59.652 | 1.00 | 43.62 | O |
| ATOM | 2902 | O | HOH | W | 26 | 36.352 | 33.342 | 61.658 | 1.00 | 55.49 | O |
| ATOM | 2903 | O | HOH | W | 27 | 44.405 | 55.158 | 53.621 | 1.00 | 49.17 | O |
| ATOM | 2904 | O | HOH | W | 28 | 44.272 | 61.824 | 55.284 | 1.00 | 46.21 | O |
| ATOM | 2905 | O | HOH | W | 29 | 42.281 | 55.531 | 68.580 | 1.00 | 38.82 | O |
| ATOM | 2906 | O | HOH | W | 30 | 27.948 | 51.240 | 68.122 | 1.00 | 39.28 | O |
| ATOM | 2907 | O | HOH | W | 31 | 35.938 | 35.788 | 80.339 | 1.00 | 48.38 | O |
| ATOM | 2908 | O | HOH | W | 32 | 48.957 | 41.809 | 74.328 | 1.00 | 56.30 | O |
| ATOM | 2909 | O | HOH | W | 33 | 49.001 | 49.292 | 84.945 | 1.00 | 28.43 | O |
| ATOM | 2910 | O | HOH | W | 34 | 51.578 | 44.648 | 73.407 | 1.00 | 43.49 | O |
| ATOM | 2911 | O | HOH | W | 35 | 33.603 | 37.124 | 52.671 | 1.00 | 46.84 | O |
| ATOM | 2912 | O | HOH | W | 36 | 26.133 | 52.272 | 56.094 | 1.00 | 63.86 | O |
| ATOM | 2913 | O | HOH | W | 37 | 48.315 | 33.115 | 63.155 | 1.00 | 48.91 | O |
| ATOM | 2914 | O | HOH | W | 38 | 34.719 | 22.759 | 91.276 | 1.00 | 70.68 | O |
| ATOM | 2915 | O | HOH | W | 39 | 34.964 | 54.812 | 73.585 | 1.00 | 57.30 | O |
| ATOM | 2916 | O | HOH | W | 40 | 38.476 | 43.002 | 75.125 | 1.00 | 53.51 | O |
| ATOM | 2917 | O | HOH | W | 41 | 50.014 | 44.374 | 83.448 | 1.00 | 55.34 | O |
| ATOM | 2918 | O | HOH | W | 42 | 45.616 | 34.739 | 67.535 | 1.00 | 59.44 | O |
| ATOM | 2919 | O | HOH | W | 43 | 33.040 | 45.363 | 71.206 | 1.00 | 41.59 | O |
| ATOM | 2920 | O | HOH | W | 44 | 39.679 | 64.344 | 60.369 | 1.00 | 50.48 | O |
| ATOM | 2921 | O | HOH | W | 45 | 29.944 | 24.531 | 54.502 | 1.00 | 57.22 | O |
| ATOM | 2922 | O | HOH | W | 46 | 35.850 | 66.577 | 49.385 | 1.00 | 61.89 | O |
| ATOM | 2923 | O | HOH | W | 47 | 34.537 | 65.545 | 58.102 | 1.00 | 50.53 | O |
| ATOM | 2924 | O | HOH | W | 48 | 57.881 | 58.562 | 72.296 | 1.00 | 58.71 | O |
| ATOM | 2925 | O | HOH | W | 49 | 45.938 | 60.009 | 66.364 | 1.00 | 45.52 | O |
| ATOM | 2926 | O | HOH | W | 50 | 35.642 | 28.396 | 67.982 | 1.00 | 60.96 | O |
| ATOM | 2927 | O | HOH | W | 51 | 45.880 | 61.279 | 63.309 | 1.00 | 46.98 | O |
| ATOM | 2928 | O | HOH | W | 52 | 40.263 | 56.865 | 79.264 | 1.00 | 50.80 | O |
| ATOM | 2929 | O | HOH | W | 53 | 43.203 | 64.012 | 54.700 | 1.00 | 51.55 | O |
| ATOM | 2930 | O | HOH | W | 54 | 36.501 | 17.930 | 84.182 | 1.00 | 48.48 | O |
| ATOM | 2931 | O | HOH | W | 55 | 34.272 | 30.746 | 61.368 | 1.00 | 50.76 | O |
| ATOM | 2932 | O | HOH | W | 56 | 32.064 | 64.858 | 65.309 | 1.00 | 50.96 | O |
| ATOM | 2933 | O | HOH | W | 57 | 36.444 | 63.696 | 56.868 | 1.00 | 52.31 | O |
| ATOM | 2934 | O | HOH | W | 58 | 39.031 | 23.728 | 96.274 | 1.00 | 57.76 | O |
| ATOM | 2935 | O | HOH | W | 59 | 50.965 | 43.327 | 67.448 | 1.00 | 72.13 | O |
| ATOM | 2936 | O | HOH | W | 60 | 36.811 | 36.321 | 46.565 | 1.00 | 54.98 | O |
| ATOM | 2937 | O | HOH | W | 61 | 39.591 | 61.315 | 73.814 | 1.00 | 56.98 | O |
| ATOM | 2938 | O | HOH | W | 62 | 30.578 | 30.711 | 67.380 | 1.00 | 58.90 | O |
| ATOM | 2939 | O | HOH | W | 63 | 37.096 | 36.149 | 90.746 | 1.00 | 63.94 | O |
| ATOM | 2940 | O | HOH | W | 64 | 53.731 | 49.039 | 71.196 | 1.00 | 46.01 | O |
| ATOM | 2941 | O | HOH | W | 65 | 64.801 | 43.240 | 70.753 | 1.00 | 70.18 | O |
| ATOM | 2942 | O | HOH | W | 66 | 54.222 | 34.578 | 97.033 | 1.00 | 69.42 | O |
| ATOM | 2943 | O | HOH | W | 67 | 51.251 | 39.244 | 74.318 | 1.00 | 55.92 | O |
| ATOM | 2944 | O | HOH | W | 68 | 46.366 | 64.258 | 63.026 | 1.00 | 52.52 | O |
| ATOM | 2945 | O | HOH | W | 69 | 34.187 | 69.148 | 63.834 | 1.00 | 48.04 | O |
| ATOM | 2946 | O | HOH | W | 70 | 29.490 | 63.480 | 55.106 | 1.00 | 57.42 | O |
| ATOM | 2947 | O | HOH | W | 71 | 37.218 | 65.999 | 68.774 | 1.00 | 60.44 | O |
| ATOM | 2948 | O | HOH | W | 72 | 36.303 | 19.025 | 86.398 | 1.00 | 56.77 | O |
| ATOM | 2949 | O | HOH | W | 73 | 60.229 | 40.564 | 98.379 | 1.00 | 52.92 | O |
| ATOM | 2950 | O | HOH | W | 74 | 27.287 | 31.349 | 53.803 | 1.00 | 53.68 | O |
| ATOM | 2951 | O | HOH | W | 75 | 48.560 | 61.940 | 59.263 | 1.00 | 63.59 | O |
| ATOM | 2952 | O | HOH | W | 76 | 42.661 | 62.719 | 60.739 | 1.00 | 55.32 | O |
| ATOM | 2953 | O | HOH | W | 77 | 29.748 | 31.557 | 60.035 | 1.00 | 56.44 | O |
| ATOM | 2954 | O | HOH | W | 78 | 39.144 | 42.750 | 44.092 | 1.00 | 54.03 | O |
| ATOM | 2955 | O | HOH | W | 79 | 40.413 | 68.093 | 49.234 | 1.00 | 60.74 | O |
| ATOM | 2956 | O | HOH | W | 80 | 40.589 | 56.359 | 95.037 | 1.00 | 63.31 | O |
| ATOM | 2957 | O | HOH | W | 81 | 40.269 | 65.093 | 55.850 | 1.00 | 56.73 | O |
| ATOM | 2958 | O | HOH | W | 82 | 52.490 | 44.985 | 69.058 | 1.00 | 70.44 | O |
| ATOM | 2959 | O | HOH | W | 83 | 38.847 | 65.219 | 58.098 | 1.00 | 54.59 | O |
| ATOM | 2960 | O | HOH | W | 84 | 43.081 | 52.100 | 48.469 | 1.00 | 79.31 | O |
| ATOM | 2961 | O | HOH | W | 85 | 59.102 | 39.678 | 100.868 | 1.00 | 59.25 | O |
| ATOM | 2962 | O | HOH | W | 86 | 37.308 | 45.216 | 76.345 | 1.00 | 71.39 | O |
| ATOM | 2963 | O | HOH | W | 87 | 48.816 | 23.504 | 92.322 | 1.00 | 61.02 | O |
| ATOM | 2964 | O | HOH | W | 88 | 32.880 | 47.007 | 54.298 | 1.00 | 39.86 | O |
| ATOM | 2965 | O | HOH | W | 89 | 26.328 | 48.899 | 55.228 | 1.00 | 59.06 | O |
| ATOM | 2966 | O | HOH | W | 90 | 47.438 | 27.437 | 86.816 | 1.00 | 59.33 | O |
| ATOM | 2967 | O | HOH | W | 91 | 48.181 | 68.466 | 65.851 | 1.00 | 62.61 | O |
| TER | 2968 | | HOH | W | 91 | | | | | | |
| ATOM | 2969 | P1 | ATP | C | 500 | 42.297 | 33.003 | 67.278 | 1.00 | 53.87 | P |
| ATOM | 2970 | O1 | ATP | C | 500 | 42.868 | 33.131 | 68.532 | 1.00 | 52.20 | O |
| ATOM | 2971 | O2 | ATP | C | 500 | 41.712 | 34.235 | 66.930 | 1.00 | 54.96 | O |
| ATOM | 2972 | O3 | ATP | C | 500 | 43.317 | 32.720 | 66.328 | 1.00 | 56.54 | O |
| ATOM | 2973 | P2 | ATP | C | 500 | 39.786 | 32.294 | 66.943 | 1.00 | 50.19 | P |
| ATOM | 2974 | O4 | ATP | C | 500 | 38.895 | 31.136 | 67.177 | 1.00 | 51.43 | O |
| ATOM | 2975 | O5 | ATP | C | 500 | 39.689 | 32.775 | 65.551 | 1.00 | 49.15 | O |
| ATOM | 2976 | O6 | ATP | C | 500 | 41.333 | 31.893 | 67.304 | 1.00 | 52.36 | O |
| ATOM | 2977 | P3 | ATP | C | 500 | 38.810 | 34.943 | 67.958 | 1.00 | 46.38 | P |
| ATOM | 2978 | O7 | ATP | C | 500 | 39.242 | 35.598 | 66.753 | 1.00 | 48.29 | O |
| ATOM | 2979 | O8 | ATP | C | 500 | 38.956 | 35.620 | 69.264 | 1.00 | 47.57 | O |
| ATOM | 2980 | O9 | ATP | C | 500 | 39.418 | 33.453 | 67.985 | 1.00 | 50.66 | O |
| ATOM | 2981 | O10 | ATP | C | 500 | 37.223 | 34.807 | 67.964 | 1.00 | 49.83 | O |
| ATOM | 2982 | C1 | ATP | C | 500 | 36.772 | 33.713 | 67.247 | 1.00 | 49.63 | C |
| ATOM | 2983 | C3 | ATP | C | 500 | 35.450 | 33.118 | 67.275 | 1.00 | 49.36 | C |
| ATOM | 2984 | O11 | ATP | C | 500 | 34.326 | 33.918 | 66.818 | 1.00 | 48.32 | O |
| ATOM | 2985 | C7 | ATP | C | 500 | 35.716 | 32.086 | 66.165 | 1.00 | 49.11 | C |
| ATOM | 2986 | O12 | ATP | C | 500 | 36.264 | 30.884 | 66.753 | 1.00 | 48.76 | O |
| ATOM | 2987 | C9 | ATP | C | 500 | 34.438 | 31.989 | 65.349 | 1.00 | 48.19 | C |
| ATOM | 2988 | O13 | ATP | C | 500 | 33.638 | 30.963 | 65.889 | 1.00 | 51.17 | O |
| ATOM | 2989 | C10 | ATP | C | 500 | 33.815 | 33.392 | 65.604 | 1.00 | 47.37 | C |
| ATOM | 2990 | N2 | ATP | C | 500 | 34.062 | 34.466 | 64.589 | 1.00 | 44.46 | N |
| ATOM | 2991 | C8 | ATP | C | 500 | 34.953 | 35.532 | 64.583 | 1.00 | 43.87 | C |
| ATOM | 2992 | N4 | ATP | C | 500 | 34.925 | 36.295 | 63.546 | 1.00 | 42.86 | N |
| ATOM | 2993 | C5 | ATP | C | 500 | 33.944 | 35.710 | 62.772 | 1.00 | 43.23 | C |
| ATOM | 2994 | C6 | ATP | C | 500 | 33.354 | 36.010 | 61.477 | 1.00 | 43.03 | C |
| ATOM | 2995 | N5 | ATP | C | 500 | 33.772 | 37.051 | 60.758 | 1.00 | 43.20 | N |
| ATOM | 2996 | N1 | ATP | C | 500 | 32.339 | 35.192 | 60.963 | 1.00 | 43.20 | N |
| ATOM | 2997 | C2 | ATP | C | 500 | 31.899 | 34.113 | 61.687 | 1.00 | 41.52 | C |
| ATOM | 2998 | N3 | ATP | C | 500 | 32.394 | 33.761 | 62.912 | 1.00 | 41.36 | N |
| ATOM | 2999 | C4 | ATP | C | 500 | 33.382 | 34.579 | 63.398 | 1.00 | 42.66 | C |
| TER | 3000 | | ATP | C | 500 | | | | | | |
| ATOM | 3001 | N1 | HET | D | 501 | 57.456 | 60.218 | 76.806 | 1.00 | 80.82 | N |
| ATOM | 3002 | C1 | HET | D | 501 | 58.047 | 59.280 | 75.918 | 1.00 | 80.59 | C |
| ATOM | 3003 | C2 | HET | D | 501 | 56.648 | 61.257 | 76.089 | 1.00 | 80.97 | C |
| ATOM | 3004 | C3 | HET | D | 501 | 57.323 | 60.183 | 78.295 | 1.00 | 80.90 | C |
| ATOM | 3005 | C4 | HET | D | 501 | 58.914 | 58.065 | 76.459 | 1.00 | 80.42 | C |
| ATOM | 3006 | O1 | HET | D | 501 | 57.874 | 59.382 | 74.700 | 1.00 | 81.08 | O |
| ATOM | 3007 | C5 | HET | D | 501 | 56.919 | 62.631 | 76.733 | 1.00 | 81.25 | C |
| ATOM | 3008 | C6 | HET | D | 501 | 55.141 | 60.865 | 76.091 | 1.00 | 80.54 | C |
| ATOM | 3009 | C7 | HET | D | 501 | 57.642 | 61.552 | 78.938 | 1.00 | 81.27 | C |
| ATOM | 3010 | C8 | HET | D | 501 | 58.006 | 56.853 | 76.918 | 1.00 | 80.33 | C |
| ATOM | 3011 | C9 | HET | D | 501 | 59.818 | 57.577 | 75.281 | 1.00 | 79.58 | C |
| ATOM | 3012 | O2 | HET | D | 501 | 59.753 | 58.463 | 77.422 | 1.00 | 80.00 | O |
| ATOM | 3013 | N2 | HET | D | 501 | 56.800 | 62.570 | 78.229 | 1.00 | 81.62 | N |
| ATOM | 3014 | C10 | HET | D | 501 | 59.138 | 61.934 | 78.846 | 1.00 | 81.10 | C |
| ATOM | 3015 | F1 | HET | D | 501 | 57.225 | 56.438 | 75.931 | 1.00 | 80.38 | F |
| ATOM | 3016 | F2 | HET | D | 501 | 58.720 | 55.827 | 77.344 | 1.00 | 81.26 | F |
| ATOM | 3017 | F3 | HET | D | 501 | 57.219 | 57.190 | 77.923 | 1.00 | 80.53 | F |
| ATOM | 3018 | C11 | HET | D | 501 | 55.921 | 63.496 | 78.865 | 1.00 | 81.57 | C |
| ATOM | 3019 | C12 | HET | D | 501 | 55.733 | 63.569 | 80.344 | 1.00 | 81.59 | C |
| ATOM | 3020 | O3 | HET | D | 501 | 55.302 | 64.278 | 78.157 | 1.00 | 81.86 | O |
| ATOM | 3021 | C13 | HET | D | 501 | 56.865 | 63.963 | 81.144 | 1.00 | 81.86 | C |
| ATOM | 3022 | C14 | HET | D | 501 | 54.433 | 63.366 | 80.947 | 1.00 | 81.43 | C |
| ATOM | 3023 | C15 | HET | D | 501 | 56.712 | 64.182 | 82.542 | 1.00 | 82.13 | C |
| ATOM | 3024 | C16 | HET | D | 501 | 54.277 | 63.591 | 82.370 | 1.00 | 81.90 | C |
| ATOM | 3025 | C17 | HET | D | 501 | 55.433 | 63.999 | 83.148 | 1.00 | 82.45 | C |
| ATOM | 3026 | C18 | HET | D | 501 | 55.342 | 64.215 | 84.568 | 1.00 | 82.83 | C |
| ATOM | 3027 | N3 | HET | D | 501 | 55.301 | 64.379 | 85.719 | 1.00 | 83.53 | N |
| TER | 3028 | | HET | D | 501 | | | | | | |
| ATOM | 3029 | MG + 2 | MG2 | E | 502 | 40.725 | 34.707 | 65.249 | 1.00 | 42.97 | MG |
| TER | 3030 | | MG2 | E | 502 | | | | | | |
| END | | | | | | | | | | | |

TABLE 6
Atomic coordinates of PDHK-2* with Compound 2

REMARK coordinates from restrained individual B-factor refinement
REMARK refinement resolution: 30 - 2.4 A

| | | |
|---|---|---|
| REMARK starting | r = 0.2476 | free_r = 0.2612 |
| REMARK final | r = 0.2473 | free_r = 0.2614 |

| | | |
|---|---|---|
| REMARK B rmsd for bonded mainchain atoms = | 2.183 | target = 1.5 |
| REMARK B rmsd for bonded sidechain atoms = | 2.932 | target = 2.0 |
| REMARK B rmsd for angle mainchain atoms = | 3.759 | target = 2.0 |
| REMARK B rmsd for angle sidechain atoms = | 4.557 | target = 2.5 |

REMARK wa = 2.02904
REMARK rweight = 8.483175E-02
REMARK target = mlf steps = 15
REMARK sg = P6(4) a = 108.873 b = 108.873 c = 84.202 alpha = 90 beta = 90 gamma = 120
REMARK parameter file 1: MSI_CNX_TOPPAR:protein_rep.param
REMARK parameter file 2: . . . /toppar/het.par
REMARK parameter file 3: MSI_CNX_TOPPAR:water_rep.param
REMARK molecular structure file: . . . /generate/generate.psf
REMARK input coordinates: minimize.pdb
REMARK reflection file = . . . /pdhk69.cv
REMARK ncs = none
REMARK B-correction resolution: 6.0 − 2.4
REMARK initial B-factor correction applied to fobs:

| | | | | | | |
|---|---|---|---|---|---|---|
| REMARK | B11 = | −3.423 | B22 = | −3.423 | B33 = | 6.845 |
| REMARK | B12 = | −5.148 | B13 = | 0.000 | B23 = | 0.000 |

REMARK B-factor correction applied to coordinate array B: −0.041
REMARK bulk solvent: (Mask) density level = 0.358118 e/A\hat{3}, B-factor = 53.3451 A\hat{2}
REMARK reflections with |Fobs|/sigma_F < 0.0 rejected
REMARK reflections with |Fobs| > 10000 * rms(Fobs) rejected

| | | |
|---|---|---|
| REMARK theoretical total number of refl.in resol.range: | 22285 | (100.0%) |
| REMARK number of unobserved reflections (no entry or \|F\| = 0): | 671 | (3.0%) |
| REMARK number of reflections rejected: | 0 | (0.0%) |
| REMARK total number of reflections used: | 21614 | (97.0%) |
| REMARK number of reflections in working set: | 20560 | (92.3%) |
| REMARK number of reflections in test set: | 1054 | (4.7%) |
| REMARK FILENAME = “bindividual.pdb” | | |
| REMARK DATE:Apr. 03, 2001 18:54:52 created by user: knoechel | | |
| REMARK Written by CNX VERSION: 2000 | | |

| | | | | | | | | | | | | |
|---|---|---|---|---|---|---|---|---|---|---|---|---|
| ATOM | 1 | C | GLY | A | 14 | 61.100 | 42.467 | 101.382 | 1.00 | 58.43 | A | C |
| ATOM | 2 | O | GLY | A | 14 | 60.442 | 42.538 | 102.419 | 1.00 | 58.34 | A | O |
| ATOM | 3 | N | GLY | A | 14 | 63.270 | 41.711 | 100.525 | 1.00 | 57.12 | A | N |
| ATOM | 4 | CA | GLY | A | 14 | 61.989 | 41.273 | 101.120 | 1.00 | 56.09 | A | C |
| ATOM | 5 | N | SER | A | 15 | 61.073 | 43.401 | 100.434 | 1.00 | 60.61 | A | N |
| ATOM | 6 | CA | SER | A | 15 | 60.263 | 44.611 | 100.565 | 1.00 | 64.13 | A | C |
| ATOM | 7 | CB | SER | A | 15 | 61.181 | 45.834 | 100.634 | 1.00 | 65.51 | A | C |
| ATOM | 8 | OG | SER | A | 15 | 62.111 | 45.820 | 99.563 | 1.00 | 69.50 | A | O |
| ATOM | 9 | C | SER | A | 15 | 59.228 | 44.812 | 99.449 | 1.00 | 64.10 | A | C |
| ATOM | 10 | O | SER | A | 15 | 58.529 | 45.830 | 99.425 | 1.00 | 65.29 | A | O |
| ATOM | 11 | N | ALA | A | 16 | 59.128 | 43.848 | 98.537 | 1.00 | 63.69 | A | N |
| ATOM | 12 | CA | ALA | A | 16 | 58.177 | 43.928 | 97.431 | 1.00 | 62.75 | A | C |
| ATOM | 13 | CB | ALA | A | 16 | 58.029 | 42.562 | 96.768 | 1.00 | 63.17 | A | C |
| ATOM | 14 | C | ALA | A | 16 | 56.803 | 44.452 | 97.851 | 1.00 | 61.47 | A | C |
| ATOM | 15 | O | ALA | A | 16 | 56.261 | 45.342 | 97.203 | 1.00 | 59.81 | A | O |
| ATOM | 16 | N | PRO | A | 17 | 56.222 | 43.904 | 98.935 | 1.00 | 61.32 | A | N |
| ATOM | 17 | CD | PRO | A | 17 | 56.693 | 42.760 | 99.737 | 1.00 | 60.32 | A | C |
| ATOM | 18 | CA | PRO | A | 17 | 54.902 | 44.358 | 99.397 | 1.00 | 62.89 | A | C |
| ATOM | 19 | CB | PRO | A | 17 | 54.634 | 43.479 | 100.620 | 1.00 | 61.76 | A | C |
| ATOM | 20 | CG | PRO | A | 17 | 55.400 | 42.218 | 100.314 | 1.00 | 60.07 | A | C |
| ATOM | 21 | C | PRO | A | 17 | 54.871 | 45.844 | 99.755 | 1.00 | 64.92 | A | C |
| ATOM | 22 | O | PRO | A | 17 | 53.832 | 46.501 | 99.642 | 1.00 | 66.07 | A | O |
| ATOM | 23 | N | LYS | A | 18 | 56.018 | 46.360 | 100.191 | 1.00 | 67.71 | A | N |
| ATOM | 24 | CA | LYS | A | 18 | 56.159 | 47.760 | 100.588 | 1.00 | 69.26 | A | C |
| ATOM | 25 | CB | LYS | A | 18 | 57.456 | 47.951 | 101.380 | 1.00 | 71.71 | A | C |
| ATOM | 26 | CG | LYS | A | 18 | 57.739 | 49.389 | 101.787 | 1.00 | 73.76 | A | C |
| ATOM | 27 | CD | LYS | A | 18 | 59.160 | 49.539 | 102.316 | 1.00 | 76.77 | A | C |
| ATOM | 28 | CE | LYS | A | 18 | 59.489 | 50.994 | 102.626 | 1.00 | 77.29 | A | C |
| ATOM | 29 | NZ | LYS | A | 18 | 60.926 | 51.178 | 102.964 | 1.00 | 77.23 | A | N |
| ATOM | 30 | C | LYS | A | 18 | 56.175 | 48.671 | 99.372 | 1.00 | 69.75 | A | C |
| ATOM | 31 | O | LYS | A | 18 | 55.481 | 49.690 | 99.336 | 1.00 | 68.18 | A | O |
| ATOM | 32 | N | TYR | A | 19 | 56.994 | 48.307 | 98.392 | 1.00 | 70.44 | A | N |
| ATOM | 33 | CA | TYR | A | 19 | 57.107 | 49.072 | 97.158 | 1.00 | 73.23 | A | C |
| ATOM | 34 | CB | TYR | A | 19 | 58.064 | 48.375 | 96.196 | 1.00 | 76.76 | A | C |
| ATOM | 35 | CG | TYR | A | 19 | 59.496 | 48.353 | 96.667 | 1.00 | 81.78 | A | C |
| ATOM | 36 | CD1 | TYR | A | 19 | 59.977 | 49.333 | 97.534 | 1.00 | 83.03 | A | C |
| ATOM | 37 | CE1 | TYR | A | 19 | 61.309 | 49.365 | 97.918 | 1.00 | 84.94 | A | C |
| ATOM | 38 | CD2 | TYR | A | 19 | 60.391 | 47.394 | 96.195 | 1.00 | 83.24 | A | C |
| ATOM | 39 | CE2 | TYR | A | 19 | 61.730 | 47.417 | 96.569 | 1.00 | 85.80 | A | C |
| ATOM | 40 | CZ | TYR | A | 19 | 62.180 | 48.405 | 97.429 | 1.00 | 86.09 | A | C |
| ATOM | 41 | OH | TYR | A | 19 | 63.507 | 48.435 | 97.786 | 1.00 | 88.94 | A | O |
| ATOM | 42 | C | TYR | A | 19 | 55.743 | 49.198 | 96.497 | 1.00 | 72.70 | A | C |
| ATOM | 43 | O | TYR | A | 19 | 55.302 | 50.288 | 96.126 | 1.00 | 72.41 | A | O |
| ATOM | 44 | N | ILE | A | 20 | 55.079 | 48.060 | 96.356 | 1.00 | 71.77 | A | N |
| ATOM | 45 | CA | ILE | A | 20 | 53.767 | 48.015 | 95.750 | 1.00 | 71.86 | A | C |
| ATOM | 46 | CB | ILE | A | 20 | 53.231 | 46.572 | 95.759 | 1.00 | 69.51 | A | C |
| ATOM | 47 | CG2 | ILE | A | 20 | 51.845 | 46.516 | 95.146 | 1.00 | 69.54 | A | C |
| ATOM | 48 | CG1 | ILE | A | 20 | 54.191 | 45.681 | 94.970 | 1.00 | 69.43 | A | C |
| ATOM | 49 | CD1 | ILE | A | 20 | 53.903 | 44.204 | 95.081 | 1.00 | 67.70 | A | C |
| ATOM | 50 | C | ILE | A | 20 | 52.814 | 48.951 | 96.488 | 1.00 | 72.05 | A | C |
| ATOM | 51 | O | ILE | A | 20 | 52.081 | 49.705 | 95.856 | 1.00 | 72.16 | A | O |
| ATOM | 52 | N | GLU | A | 21 | 52.835 | 48.919 | 97.818 | 1.00 | 73.02 | A | N |
| ATOM | 53 | CA | GLU | A | 21 | 51.961 | 49.786 | 98.607 | 1.00 | 74.21 | A | C |
| ATOM | 54 | CB | GLU | A | 21 | 52.082 | 49.469 | 100.100 | 1.00 | 77.26 | A | C |
| ATOM | 55 | CG | GLU | A | 21 | 51.313 | 50.433 | 101.006 | 1.00 | 83.31 | A | C |
| ATOM | 56 | CD | GLU | A | 21 | 49.853 | 50.579 | 100.604 | 1.00 | 87.34 | A | C |
| ATOM | 57 | OE1 | GLU | A | 21 | 49.152 | 49.546 | 100.531 | 1.00 | 89.27 | A | O |
| ATOM | 58 | OE2 | GLU | A | 21 | 49.408 | 51.726 | 100.361 | 1.00 | 88.30 | A | O |
| ATOM | 59 | C | GLU | A | 21 | 52.311 | 51.252 | 98.369 | 1.00 | 73.07 | A | C |
| ATOM | 60 | O | GLU | A | 21 | 51.434 | 52.115 | 98.364 | 1.00 | 73.62 | A | O |
| ATOM | 61 | N | HIS | A | 22 | 53.594 | 51.529 | 98.167 | 1.00 | 70.79 | A | N |
| ATOM | 62 | CA | HIS | A | 22 | 54.038 | 52.890 | 97.930 | 1.00 | 69.32 | A | C |
| ATOM | 63 | CB | HIS | A | 22 | 55.567 | 52.957 | 97.936 | 1.00 | 67.60 | A | C |
| ATOM | 64 | CG | HIS | A | 22 | 56.113 | 54.291 | 97.536 | 1.00 | 69.05 | A | C |
| ATOM | 65 | CD2 | HIS | A | 22 | 57.002 | 54.641 | 96.575 | 1.00 | 68.84 | A | C |
| ATOM | 66 | ND1 | HIS | A | 22 | 55.730 | 55.466 | 98.149 | 1.00 | 69.20 | A | N |
| ATOM | 67 | CE1 | HIS | A | 22 | 56.357 | 56.481 | 97.581 | 1.00 | 69.83 | A | C |
| ATOM | 68 | NE2 | HIS | A | 22 | 57.135 | 56.007 | 96.622 | 1.00 | 68.43 | A | N |
| ATOM | 69 | C | HIS | A | 22 | 53.508 | 53.400 | 96.597 | 1.00 | 68.42 | A | C |
| ATOM | 70 | O | HIS | A | 22 | 52.618 | 54.251 | 96.554 | 1.00 | 68.63 | A | O |
| ATOM | 71 | N | PHE | A | 23 | 54.064 | 52.869 | 95.511 | 1.00 | 67.00 | A | N |
| ATOM | 72 | CA | PHE | A | 23 | 53.677 | 53.278 | 94.168 | 1.00 | 64.70 | A | C |
| ATOM | 73 | CB | PHE | A | 23 | 54.536 | 52.555 | 93.134 | 1.00 | 61.18 | A | C |
| ATOM | 74 | CG | PHE | A | 23 | 55.987 | 52.904 | 93.213 | 1.00 | 58.02 | A | C |
| ATOM | 75 | CD1 | PHE | A | 23 | 56.881 | 52.071 | 93.876 | 1.00 | 58.38 | A | C |
| ATOM | 76 | CD2 | PHE | A | 23 | 56.459 | 54.088 | 92.655 | 1.00 | 56.75 | A | C |
| ATOM | 77 | CE1 | PHE | A | 23 | 58.233 | 52.414 | 93.980 | 1.00 | 57.70 | A | C |
| ATOM | 78 | CE2 | PHE | A | 23 | 57.803 | 54.441 | 92.755 | 1.00 | 55.33 | A | C |
| ATOM | 79 | CZ | PHE | A | 23 | 58.692 | 53.604 | 93.418 | 1.00 | 55.87 | A | C |
| ATOM | 80 | C | PHE | A | 23 | 52.201 | 53.084 | 93.846 | 1.00 | 64.21 | A | C |
| ATOM | 81 | O | PHE | A | 23 | 51.677 | 53.733 | 92.947 | 1.00 | 63.44 | A | O |
| ATOM | 82 | N | SER | A | 24 | 51.535 | 52.203 | 94.584 | 1.00 | 65.63 | A | N |
| ATOM | 83 | CA | SER | A | 24 | 50.120 | 51.938 | 94.368 | 1.00 | 67.71 | A | C |
| ATOM | 84 | CB | SER | A | 24 | 49.703 | 50.653 | 95.091 | 1.00 | 68.51 | A | C |
| ATOM | 85 | OG | SER | A | 24 | 48.591 | 50.864 | 95.947 | 1.00 | 69.14 | A | O |
| ATOM | 86 | C | SER | A | 24 | 49.277 | 53.103 | 94.866 | 1.00 | 69.39 | A | C |
| ATOM | 87 | O | SER | A | 24 | 48.048 | 53.085 | 94.758 | 1.00 | 69.16 | A | O |
| ATOM | 88 | N | LYS | A | 25 | 49.940 | 54.112 | 95.421 | 1.00 | 71.87 | A | N |
| ATOM | 89 | CA | LYS | A | 25 | 49.243 | 55.282 | 95.932 | 1.00 | 75.18 | A | C |
| ATOM | 90 | CB | LYS | A | 25 | 49.987 | 55.852 | 97.143 | 1.00 | 80.42 | A | C |
| ATOM | 91 | CG | LYS | A | 25 | 49.895 | 54.969 | 98.379 | 1.00 | 86.33 | A | C |
| ATOM | 92 | CD | LYS | A | 25 | 50.737 | 55.506 | 99.518 | 1.00 | 92.50 | A | C |
| ATOM | 93 | CE | LYS | A | 25 | 50.651 | 54.594 | 100.734 | 1.00 | 96.70 | A | C |
| ATOM | 94 | NZ | LYS | A | 25 | 51.585 | 55.019 | 101.816 | 1.00 | 100.47 | A | N |
| ATOM | 95 | C | LYS | A | 25 | 49.128 | 56.327 | 94.834 | 1.00 | 73.51 | A | C |
| ATOM | 96 | O | LYS | A | 25 | 48.266 | 57.198 | 94.883 | 1.00 | 73.28 | A | O |
| ATOM | 97 | N | PHE | A | 26 | 50.005 | 56.222 | 93.842 | 1.00 | 72.50 | A | N |
| ATOM | 98 | CA | PHE | A | 26 | 50.010 | 57.131 | 92.705 | 1.00 | 71.66 | A | C |
| ATOM | 99 | CB | PHE | A | 26 | 51.418 | 57.248 | 92.118 | 1.00 | 75.10 | A | C |
| ATOM | 100 | CG | PHE | A | 26 | 52.425 | 57.847 | 93.056 | 1.00 | 81.43 | A | C |
| ATOM | 101 | CD1 | PHE | A | 26 | 52.742 | 57.219 | 94.260 | 1.00 | 82.88 | A | C |
| ATOM | 102 | CD2 | PHE | A | 26 | 53.064 | 59.042 | 92.732 | 1.00 | 82.57 | A | C |
| ATOM | 103 | CE1 | PHE | A | 26 | 53.684 | 57.770 | 95.125 | 1.00 | 84.28 | A | C |
| ATOM | 104 | CE2 | PHE | A | 26 | 54.006 | 59.602 | 93.589 | 1.00 | 83.74 | A | C |
| ATOM | 105 | CZ | PHE | A | 26 | 54.317 | 58.967 | 94.788 | 1.00 | 84.66 | A | C |
| ATOM | 106 | C | PHE | A | 26 | 49.086 | 56.587 | 91.618 | 1.00 | 69.35 | A | C |
| ATOM | 107 | O | PHE | A | 26 | 48.800 | 55.388 | 91.575 | 1.00 | 68.38 | A | O |
| ATOM | 108 | N | SER | A | 27 | 48.619 | 57.469 | 90.743 | 1.00 | 64.66 | A | N |
| ATOM | 109 | CA | SER | A | 27 | 47.772 | 57.047 | 89.639 | 1.00 | 59.39 | A | C |
| ATOM | 110 | CB | SER | A | 27 | 46.562 | 57.960 | 89.497 | 1.00 | 59.11 | A | C |
| ATOM | 111 | OG | SER | A | 27 | 45.570 | 57.614 | 90.447 | 1.00 | 61.46 | A | O |
| ATOM | 112 | C | SER | A | 27 | 48.616 | 57.113 | 88.383 | 1.00 | 55.83 | A | C |
| ATOM | 113 | O | SER | A | 27 | 49.452 | 57.999 | 88.251 | 1.00 | 53.97 | A | O |
| ATOM | 114 | N | PRO | A | 28 | 48.445 | 56.149 | 87.461 | 1.00 | 53.49 | A | N |
| ATOM | 115 | CD | PRO | A | 28 | 47.615 | 54.933 | 87.484 | 1.00 | 50.55 | A | C |
| ATOM | 116 | CA | PRO | A | 28 | 49.255 | 56.213 | 86.240 | 1.00 | 51.80 | A | C |
| ATOM | 117 | CB | PRO | A | 28 | 48.879 | 54.926 | 85.504 | 1.00 | 51.90 | A | C |
| ATOM | 118 | CG | PRO | A | 28 | 47.514 | 54.606 | 86.024 | 1.00 | 52.23 | A | C |
| ATOM | 119 | C | PRO | A | 28 | 48.908 | 57.485 | 85.463 | 1.00 | 50.11 | A | C |
| ATOM | 120 | O | PRO | A | 28 | 47.776 | 57.957 | 85.512 | 1.00 | 49.26 | A | O |
| ATOM | 121 | N | SER | A | 29 | 49.888 | 58.041 | 84.762 | 1.00 | 48.84 | A | N |
| ATOM | 122 | CA | SER | A | 29 | 49.686 | 59.272 | 84.015 | 1.00 | 49.08 | A | C |
| ATOM | 123 | CB | SER | A | 29 | 50.915 | 60.166 | 84.158 | 1.00 | 48.60 | A | C |
| ATOM | 124 | OG | SER | A | 29 | 51.097 | 60.534 | 85.512 | 1.00 | 53.56 | A | O |
| ATOM | 125 | C | SER | A | 29 | 49.411 | 59.016 | 82.548 | 1.00 | 47.26 | A | C |
| ATOM | 126 | O | SER | A | 29 | 50.314 | 58.660 | 81.793 | 1.00 | 45.82 | A | O |
| ATOM | 127 | N | PRO | A | 30 | 48.158 | 59.225 | 82.117 | 1.00 | 45.16 | A | N |
| ATOM | 128 | CD | PRO | A | 30 | 46.975 | 59.583 | 82.913 | 1.00 | 45.05 | A | C |
| ATOM | 129 | CA | PRO | A | 30 | 47.800 | 58.998 | 80.715 | 1.00 | 45.33 | A | C |
| ATOM | 130 | CB | PRO | A | 30 | 46.283 | 59.200 | 80.695 | 1.00 | 44.68 | A | C |
| ATOM | 131 | CG | PRO | A | 30 | 45.862 | 58.938 | 82.116 | 1.00 | 47.41 | A | C |
| ATOM | 132 | C | PRO | A | 30 | 48.505 | 59.956 | 79.773 | 1.00 | 45.33 | A | C |
| ATOM | 133 | O | PRO | A | 30 | 48.493 | 61.154 | 79.986 | 1.00 | 46.31 | A | O |
| ATOM | 134 | N | LEU | A | 31 | 49.121 | 59.416 | 78.731 | 1.00 | 44.58 | A | N |
| ATOM | 135 | CA | LEU | A | 31 | 49.800 | 60.223 | 77.739 | 1.00 | 46.04 | A | C |
| ATOM | 136 | CB | LEU | A | 31 | 51.198 | 59.689 | 77.484 | 1.00 | 46.42 | A | C |
| ATOM | 137 | CG | LEU | A | 31 | 52.285 | 60.383 | 78.295 | 1.00 | 52.41 | A | C |
| ATOM | 138 | CD1 | LEU | A | 31 | 53.131 | 59.369 | 79.029 | 1.00 | 53.05 | A | C |
| ATOM | 139 | CD2 | LEU | A | 31 | 53.120 | 61.209 | 77.348 | 1.00 | 55.71 | A | C |
| ATOM | 140 | C | LEU | A | 31 | 49.001 | 60.200 | 76.454 | 1.00 | 48.44 | A | C |
| ATOM | 141 | O | LEU | A | 31 | 48.322 | 59.214 | 76.152 | 1.00 | 50.61 | A | O |
| ATOM | 142 | N | SER | A | 32 | 49.093 | 61.284 | 75.693 | 1.00 | 47.36 | A | N |
| ATOM | 143 | CA | SER | A | 32 | 48.361 | 61.397 | 74.442 | 1.00 | 45.93 | A | C |
| ATOM | 144 | CB | SER | A | 32 | 47.885 | 62.827 | 74.218 | 1.00 | 42.68 | A | C |
| ATOM | 145 | OG | SER | A | 32 | 48.911 | 63.571 | 73.579 | 1.00 | 43.74 | A | O |
| ATOM | 146 | C | SER | A | 32 | 49.290 | 61.039 | 73.304 | 1.00 | 46.32 | A | C |
| ATOM | 147 | O | SER | A | 32 | 50.507 | 61.137 | 73.424 | 1.00 | 45.17 | A | O |
| ATOM | 148 | N | MET | A | 33 | 48.705 | 60.665 | 72.180 | 1.00 | 47.77 | A | N |
| ATOM | 149 | CA | MET | A | 33 | 49.500 | 60.304 | 71.036 | 1.00 | 49.57 | A | C |
| ATOM | 150 | CB | MET | A | 33 | 48.585 | 59.832 | 69.910 | 1.00 | 52.07 | A | C |
| ATOM | 151 | CG | MET | A | 33 | 49.315 | 59.154 | 68.779 | 1.00 | 56.59 | A | C |
| ATOM | 152 | SD | MET | A | 33 | 48.718 | 57.465 | 68.590 | 1.00 | 64.67 | A | S |
| ATOM | 153 | CE | MET | A | 33 | 50.219 | 56.618 | 68.387 | 1.00 | 62.37 | A | C |
| ATOM | 154 | C | MET | A | 33 | 50.370 | 61.472 | 70.568 | 1.00 | 50.80 | A | C |
| ATOM | 155 | O | MET | A | 33 | 51.377 | 61.257 | 69.878 | 1.00 | 50.03 | A | O |
| ATOM | 156 | N | LYS | A | 34 | 49.961 | 62.694 | 70.922 | 1.00 | 52.93 | A | N |
| ATOM | 157 | CA | LYS | A | 34 | 50.786 | 63.820 | 70.496 | 1.00 | 55.80 | A | C |
| ATOM | 158 | CB | LYS | A | 34 | 49.995 | 65.128 | 70.396 | 1.00 | 58.14 | A | C |
| ATOM | 159 | CG | LYS | A | 34 | 50.547 | 66.035 | 69.285 | 1.00 | 60.20 | A | C |
| ATOM | 160 | CD | LYS | A | 34 | 49.715 | 67.276 | 69.076 | 1.00 | 63.46 | A | C |
| ATOM | 161 | CE | LYS | A | 34 | 50.174 | 68.399 | 69.979 | 1.00 | 66.10 | A | C |
| ATOM | 162 | NZ | LYS | A | 34 | 49.068 | 69.375 | 70.192 | 1.00 | 68.35 | A | N |
| ATOM | 163 | C | LYS | A | 34 | 51.987 | 64.017 | 71.390 | 1.00 | 54.76 | A | C |
| ATOM | 164 | O | LYS | A | 34 | 53.079 | 64.282 | 70.889 | 1.00 | 52.26 | A | O |
| ATOM | 165 | N | GLN | A | 35 | 51.801 | 63.901 | 72.707 | 1.00 | 55.63 | A | N |
| ATOM | 166 | CA | GLN | A | 35 | 52.951 | 64.024 | 73.608 | 1.00 | 57.14 | A | C |
| ATOM | 167 | CB | GLN | A | 35 | 52.541 | 63.855 | 75.071 | 1.00 | 55.12 | A | C |
| ATOM | 168 | CG | GLN | A | 35 | 51.088 | 63.500 | 75.292 | 1.00 | 59.90 | A | C |
| ATOM | 169 | CD | GLN | A | 35 | 50.496 | 64.154 | 76.522 | 1.00 | 60.00 | A | C |
| ATOM | 170 | OE1 | GLN | A | 35 | 49.431 | 63.752 | 77.010 | 1.00 | 60.31 | A | O |
| ATOM | 171 | NE2 | GLN | A | 35 | 51.176 | 65.174 | 77.028 | 1.00 | 61.28 | A | N |
| ATOM | 172 | C | GLN | A | 35 | 53.988 | 62.967 | 73.212 | 1.00 | 58.61 | A | C |
| ATOM | 173 | O | GLN | A | 35 | 55.178 | 63.236 | 73.225 | 1.00 | 61.44 | A | O |
| ATOM | 174 | N | PHE | A | 36 | 53.556 | 61.778 | 72.800 | 1.00 | 58.67 | A | N |
| ATOM | 175 | CA | PHE | A | 36 | 54.463 | 60.703 | 72.376 | 1.00 | 61.74 | A | C |
| ATOM | 176 | CB | PHE | A | 36 | 53.689 | 59.418 | 72.034 | 1.00 | 58.21 | A | C |
| ATOM | 177 | CG | PHE | A | 36 | 53.298 | 58.593 | 73.224 | 1.00 | 56.43 | A | C |
| ATOM | 178 | CD1 | PHE | A | 36 | 51.959 | 58.287 | 73.446 | 1.00 | 52.02 | A | C |
| ATOM | 179 | CD2 | PHE | A | 36 | 54.256 | 58.078 | 74.097 | 1.00 | 54.54 | A | C |
| ATOM | 180 | CE1 | PHE | A | 36 | 51.574 | 57.484 | 74.501 | 1.00 | 51.14 | A | C |
| ATOM | 181 | CE2 | PHE | A | 36 | 53.873 | 57.263 | 75.169 | 1.00 | 51.70 | A | C |
| ATOM | 182 | CZ | PHE | A | 36 | 52.536 | 56.967 | 75.370 | 1.00 | 52.76 | A | C |
| ATOM | 183 | C | PHE | A | 36 | 55.221 | 61.118 | 71.126 | 1.00 | 65.46 | A | C |
| ATOM | 184 | O | PHE | A | 36 | 56.387 | 60.744 | 70.915 | 1.00 | 64.56 | A | O |
| ATOM | 185 | N | LEU | A | 37 | 54.545 | 61.900 | 70.294 | 1.00 | 71.44 | A | N |
| ATOM | 186 | CA | LEU | A | 37 | 55.139 | 62.359 | 69.053 | 1.00 | 77.64 | A | C |
| ATOM | 187 | CB | LEU | A | 37 | 54.048 | 62.808 | 68.086 | 1.00 | 75.76 | A | C |
| ATOM | 188 | CG | LEU | A | 37 | 54.344 | 62.600 | 66.598 | 1.00 | 73.71 | A | C |
| ATOM | 189 | CD1 | LEU | A | 37 | 54.987 | 61.238 | 66.384 | 1.00 | 72.59 | A | C |
| ATOM | 190 | CD2 | LEU | A | 37 | 53.058 | 62.728 | 65.802 | 1.00 | 74.31 | A | C |
| ATOM | 191 | C | LEU | A | 37 | 56.173 | 63.467 | 69.228 | 1.00 | 81.48 | A | C |
| ATOM | 192 | O | LEU | A | 37 | 57.159 | 63.511 | 68.496 | 1.00 | 82.03 | A | O |
| ATOM | 193 | N | ASP | A | 38 | 55.949 | 64.356 | 70.188 | 1.00 | 86.73 | A | N |
| ATOM | 194 | CA | ASP | A | 38 | 56.881 | 65.434 | 70.449 | 1.00 | 91.57 | A | C |
| ATOM | 195 | CB | ASP | A | 38 | 56.163 | 66.606 | 71.122 | 1.00 | 94.12 | A | C |
| ATOM | 196 | CG | ASP | A | 38 | 54.962 | 67.099 | 70.310 | 1.00 | 96.20 | A | C |
| ATOM | 197 | OD1 | ASP | A | 38 | 55.030 | 67.120 | 69.061 | 1.00 | 96.83 | A | O |
| ATOM | 198 | OD2 | ASP | A | 38 | 53.951 | 67.491 | 70.922 | 1.00 | 96.56 | A | O |
| ATOM | 199 | C | ASP | A | 38 | 57.940 | 64.843 | 71.347 | 1.00 | 93.24 | A | C |
| ATOM | 200 | O | ASP | A | 38 | 57.931 | 65.034 | 72.570 | 1.00 | 93.98 | A | O |
| ATOM | 201 | N | PHE | A | 39 | 58.805 | 64.058 | 70.710 | 1.00 | 94.59 | A | N |
| ATOM | 202 | CA | PHE | A | 39 | 59.913 | 63.367 | 71.363 | 1.00 | 94.79 | A | C |
| ATOM | 203 | CB | PHE | A | 39 | 59.457 | 62.182 | 72.218 | 1.00 | 94.84 | A | C |
| ATOM | 204 | CG | PHE | A | 39 | 59.864 | 62.278 | 73.640 | 1.00 | 93.53 | A | C |
| ATOM | 205 | CD1 | PHE | A | 39 | 59.103 | 63.014 | 74.538 | 1.00 | 92.70 | A | C |
| ATOM | 206 | CD2 | PHE | A | 39 | 61.066 | 61.715 | 74.067 | 1.00 | 93.16 | A | C |
| ATOM | 207 | CE1 | PHE | A | 39 | 59.531 | 63.184 | 75.848 | 1.00 | 92.99 | A | C |
| ATOM | 208 | CE2 | PHE | A | 39 | 61.500 | 61.884 | 75.394 | 1.00 | 92.60 | A | C |
| ATOM | 209 | CZ | PHE | A | 39 | 60.730 | 62.632 | 76.274 | 1.00 | 92.31 | A | C |
| ATOM | 210 | C | PHE | A | 39 | 60.763 | 62.852 | 70.220 | 1.00 | 95.48 | A | C |
| ATOM | 211 | O | PHE | A | 39 | 60.364 | 62.967 | 69.056 | 1.00 | 95.82 | A | O |
| ATOM | 212 | N | ALA | A | 44 | 65.623 | 63.799 | 72.087 | 1.00 | 89.24 | A | N |
| ATOM | 213 | CA | ALA | A | 44 | 64.949 | 64.735 | 72.981 | 1.00 | 88.53 | A | C |
| ATOM | 214 | CB | ALA | A | 44 | 63.443 | 64.717 | 72.727 | 1.00 | 89.17 | A | C |
| ATOM | 215 | C | ALA | A | 44 | 65.243 | 64.363 | 74.426 | 1.00 | 88.08 | A | C |
| ATOM | 216 | O | ALA | A | 44 | 64.368 | 64.433 | 75.289 | 1.00 | 87.89 | A | O |
| ATOM | 217 | N | CYS | A | 45 | 66.488 | 63.961 | 74.670 | 1.00 | 87.62 | A | N |
| ATOM | 218 | CA | CYS | A | 45 | 66.954 | 63.572 | 75.997 | 1.00 | 86.02 | A | C |
| ATOM | 219 | CB | CYS | A | 45 | 66.803 | 64.736 | 76.986 | 1.00 | 87.04 | A | C |
| ATOM | 220 | SG | CYS | A | 45 | 68.279 | 65.759 | 77.168 | 1.00 | 89.32 | A | S |
| ATOM | 221 | C | CYS | A | 45 | 66.288 | 62.341 | 76.589 | 1.00 | 83.74 | A | C |
| ATOM | 222 | O | CYS | A | 45 | 65.136 | 62.386 | 77.016 | 1.00 | 82.85 | A | O |
| ATOM | 223 | N | GLU | A | 46 | 67.022 | 61.237 | 76.614 | 1.00 | 81.58 | A | N |
| ATOM | 224 | CA | GLU | A | 46 | 66.495 | 60.025 | 77.207 | 1.00 | 81.02 | A | C |
| ATOM | 225 | CB | GLU | A | 46 | 67.274 | 58.803 | 76.738 | 1.00 | 81.09 | A | C |
| ATOM | 226 | CG | GLU | A | 46 | 68.753 | 59.002 | 76.637 | 1.00 | 81.79 | A | C |
| ATOM | 227 | CD | GLU | A | 46 | 69.439 | 57.773 | 76.095 | 1.00 | 81.83 | A | C |
| ATOM | 228 | OE1 | GLU | A | 46 | 69.335 | 56.708 | 76.743 | 1.00 | 79.93 | A | O |
| ATOM | 229 | OE2 | GLU | A | 46 | 70.072 | 57.873 | 75.022 | 1.00 | 82.60 | A | O |
| ATOM | 230 | C | GLU | A | 46 | 66.623 | 60.189 | 78.706 | 1.00 | 80.66 | A | C |
| ATOM | 231 | O | GLU | A | 46 | 66.098 | 59.391 | 79.482 | 1.00 | 80.65 | A | O |
| ATOM | 232 | N | LYS | A | 47 | 67.340 | 61.236 | 79.105 | 1.00 | 80.29 | A | N |
| ATOM | 233 | CA | LYS | A | 47 | 67.512 | 61.539 | 80.516 | 1.00 | 79.26 | A | C |
| ATOM | 234 | CB | LYS | A | 47 | 68.430 | 62.755 | 80.711 | 1.00 | 81.15 | A | C |
| ATOM | 235 | CG | LYS | A | 47 | 69.797 | 62.643 | 80.044 | 1.00 | 81.97 | A | C |
| ATOM | 236 | CD | LYS | A | 47 | 70.673 | 63.848 | 80.374 | 1.00 | 83.88 | A | C |
| ATOM | 237 | CE | LYS | A | 47 | 70.016 | 65.157 | 79.955 | 1.00 | 84.43 | A | C |
| ATOM | 238 | NZ | LYS | A | 47 | 70.857 | 66.338 | 80.299 | 1.00 | 85.05 | A | N |
| ATOM | 239 | C | LYS | A | 47 | 66.102 | 61.883 | 80.970 | 1.00 | 77.16 | A | C |
| ATOM | 240 | O | LYS | A | 47 | 65.612 | 61.367 | 81.975 | 1.00 | 75.65 | A | O |
| ATOM | 241 | N | THR | A | 48 | 65.454 | 62.749 | 80.191 | 1.00 | 75.36 | A | N |
| ATOM | 242 | CA | THR | A | 48 | 64.093 | 63.189 | 80.469 | 1.00 | 74.01 | A | C |
| ATOM | 243 | CB | THR | A | 48 | 63.575 | 64.124 | 79.364 | 1.00 | 75.82 | A | C |
| ATOM | 244 | OG1 | THR | A | 48 | 64.461 | 65.244 | 79.225 | 1.00 | 77.57 | A | O |
| ATOM | 245 | CG2 | THR | A | 48 | 62.180 | 64.618 | 79.706 | 1.00 | 75.18 | A | C |
| ATOM | 246 | C | THR | A | 48 | 63.168 | 61.985 | 80.553 | 1.00 | 72.22 | A | C |
| ATOM | 247 | O | THR | A | 48 | 62.281 | 61.932 | 81.406 | 1.00 | 71.47 | A | O |
| ATOM | 248 | N | SER | A | 49 | 63.383 | 61.017 | 79.665 | 1.00 | 70.10 | A | N |
| ATOM | 249 | CA | SER | A | 49 | 62.569 | 59.808 | 79.653 | 1.00 | 68.66 | A | C |
| ATOM | 250 | CB | SER | A | 49 | 62.800 | 59.015 | 78.368 | 1.00 | 67.16 | A | C |
| ATOM | 251 | OG | SER | A | 49 | 62.048 | 57.818 | 78.393 | 1.00 | 63.87 | A | O |
| ATOM | 252 | C | SER | A | 49 | 62.899 | 58.927 | 80.849 | 1.00 | 68.86 | A | C |
| ATOM | 253 | O | SER | A | 49 | 61.996 | 58.469 | 81.558 | 1.00 | 68.81 | A | O |
| ATOM | 254 | N | PHE | A | 50 | 64.197 | 58.695 | 81.060 | 1.00 | 68.47 | A | N |
| ATOM | 255 | CA | PHE | A | 50 | 64.687 | 57.867 | 82.162 | 1.00 | 67.05 | A | C |
| ATOM | 256 | CB | PHE | A | 50 | 66.214 | 57.966 | 82.268 | 1.00 | 70.16 | A | C |
| ATOM | 257 | CG | PHE | A | 50 | 66.784 | 57.286 | 83.486 | 1.00 | 73.82 | A | C |
| ATOM | 258 | CD1 | PHE | A | 50 | 66.641 | 55.907 | 83.666 | 1.00 | 74.09 | A | C |
| ATOM | 259 | CD2 | PHE | A | 50 | 67.440 | 58.025 | 84.468 | 1.00 | 72.64 | A | C |
| ATOM | 260 | CE1 | PHE | A | 50 | 67.139 | 55.275 | 84.803 | 1.00 | 73.65 | A | C |
| ATOM | 261 | CE2 | PHE | A | 50 | 67.943 | 57.401 | 85.612 | 1.00 | 75.29 | A | C |
| ATOM | 262 | CZ | PHE | A | 50 | 67.791 | 56.021 | 85.779 | 1.00 | 74.53 | A | C |
| ATOM | 263 | C | PHE | A | 50 | 64.062 | 58.272 | 83.485 | 1.00 | 65.18 | A | C |
| ATOM | 264 | O | PHE | A | 50 | 63.471 | 57.447 | 84.183 | 1.00 | 64.31 | A | O |
| ATOM | 265 | N | THR | A | 51 | 64.196 | 59.546 | 83.830 | 1.00 | 63.73 | A | N |
| ATOM | 266 | CA | THR | A | 51 | 63.638 | 60.047 | 85.078 | 1.00 | 63.84 | A | C |
| ATOM | 267 | CB | THR | A | 51 | 63.963 | 61.538 | 85.276 | 1.00 | 64.18 | A | C |
| ATOM | 268 | OG1 | THR | A | 51 | 63.719 | 62.252 | 84.057 | 1.00 | 67.53 | A | O |
| ATOM | 269 | CG2 | THR | A | 51 | 65.416 | 61.710 | 85.665 | 1.00 | 66.23 | A | C |
| ATOM | 270 | C | THR | A | 51 | 62.128 | 59.853 | 85.118 | 1.00 | 62.20 | A | C |
| ATOM | 271 | O | THR | A | 51 | 61.560 | 59.588 | 86.179 | 1.00 | 62.21 | A | O |
| ATOM | 272 | N | PHE | A | 52 | 61.473 | 59.981 | 83.964 | 1.00 | 60.58 | A | N |
| ATOM | 273 | CA | PHE | A | 52 | 60.023 | 59.802 | 83.910 | 1.00 | 58.33 | A | C |
| ATOM | 274 | CB | PHE | A | 52 | 59.470 | 60.305 | 82.578 | 1.00 | 58.41 | A | C |
| ATOM | 275 | CG | PHE | A | 52 | 58.015 | 59.957 | 82.350 | 1.00 | 57.27 | A | C |
| ATOM | 276 | CD1 | PHE | A | 52 | 57.016 | 60.539 | 83.122 | 1.00 | 55.83 | A | C |
| ATOM | 277 | CD2 | PHE | A | 52 | 57.652 | 59.046 | 81.360 | 1.00 | 56.26 | A | C |
| ATOM | 278 | CE1 | PHE | A | 52 | 55.673 | 60.226 | 82.907 | 1.00 | 59.16 | A | C |
| ATOM | 279 | CE2 | PHE | A | 52 | 56.317 | 58.725 | 81.136 | 1.00 | 56.95 | A | C |
| ATOM | 280 | CZ | PHE | A | 52 | 55.323 | 59.315 | 81.911 | 1.00 | 59.15 | A | C |
| ATOM | 281 | C | PHE | A | 52 | 59.633 | 58.334 | 84.078 | 1.00 | 57.15 | A | C |
| ATOM | 282 | O | PHE | A | 52 | 58.811 | 57.995 | 84.934 | 1.00 | 55.91 | A | O |
| ATOM | 283 | N | LEU | A | 53 | 60.220 | 57.467 | 83.257 | 1.00 | 56.37 | A | N |
| ATOM | 284 | CA | LEU | A | 53 | 59.904 | 56.044 | 83.320 | 1.00 | 60.16 | A | C |
| ATOM | 285 | CB | LEU | A | 53 | 60.599 | 55.311 | 82.169 | 1.00 | 58.53 | A | C |
| ATOM | 286 | CG | LEU | A | 53 | 60.018 | 55.657 | 80.793 | 1.00 | 58.48 | A | C |
| ATOM | 287 | CD1 | LEU | A | 53 | 60.758 | 54.900 | 79.692 | 1.00 | 56.61 | A | C |
| ATOM | 288 | CD2 | LEU | A | 53 | 58.526 | 55.320 | 80.788 | 1.00 | 55.18 | A | C |
| ATOM | 289 | C | LEU | A | 53 | 60.234 | 55.390 | 84.662 | 1.00 | 61.69 | A | C |
| ATOM | 290 | O | LEU | A | 53 | 59.602 | 54.404 | 85.044 | 1.00 | 63.12 | A | O |
| ATOM | 291 | N | ARG | A | 54 | 61.215 | 55.948 | 85.371 | 1.00 | 63.92 | A | N |
| ATOM | 292 | CA | ARG | A | 54 | 61.625 | 55.449 | 86.688 | 1.00 | 64.85 | A | C |
| ATOM | 293 | CB | ARG | A | 54 | 62.727 | 56.327 | 87.278 | 1.00 | 68.49 | A | C |
| ATOM | 294 | CG | ARG | A | 54 | 64.111 | 56.081 | 86.741 | 1.00 | 73.68 | A | C |
| ATOM | 295 | CD | ARG | A | 54 | 65.039 | 57.227 | 87.122 | 1.00 | 79.20 | A | C |
| ATOM | 296 | NE | ARG | A | 54 | 64.865 | 57.676 | 88.504 | 1.00 | 81.99 | A | N |
| ATOM | 297 | CZ | ARG | A | 54 | 65.655 | 58.563 | 89.104 | 1.00 | 84.88 | A | C |
| ATOM | 298 | NH1 | ARG | A | 54 | 66.679 | 59.094 | 88.448 | 1.00 | 85.61 | A | N |
| ATOM | 299 | NH2 | ARG | A | 54 | 65.417 | 58.927 | 90.358 | 1.00 | 85.94 | A | N |
| ATOM | 300 | C | ARG | A | 54 | 60.454 | 55.467 | 87.657 | 1.00 | 63.34 | A | C |
| ATOM | 301 | O | ARG | A | 54 | 60.337 | 54.600 | 88.515 | 1.00 | 62.51 | A | O |
| ATOM | 302 | N | GLN | A | 55 | 59.599 | 56.475 | 87.529 | 1.00 | 62.03 | A | N |
| ATOM | 303 | CA | GLN | A | 55 | 58.444 | 56.601 | 88.409 | 1.00 | 61.80 | A | C |
| ATOM | 304 | CB | GLN | A | 55 | 58.230 | 58.079 | 88.782 | 1.00 | 62.22 | A | C |
| ATOM | 305 | CG | GLN | A | 55 | 56.890 | 58.366 | 89.456 | 1.00 | 65.68 | A | C |
| ATOM | 306 | CD | GLN | A | 55 | 56.870 | 58.038 | 90.944 | 1.00 | 67.83 | A | C |
| ATOM | 307 | OE1 | GLN | A | 55 | 57.637 | 57.202 | 91.421 | 1.00 | 68.56 | A | O |
| ATOM | 308 | NE2 | GLN | A | 55 | 55.973 | 58.689 | 91.680 | 1.00 | 68.54 | A | N |
| ATOM | 309 | C | GLN | A | 55 | 57.161 | 56.034 | 87.796 | 1.00 | 59.53 | A | C |
| ATOM | 310 | O | GLN | A | 55 | 56.353 | 55.420 | 88.495 | 1.00 | 60.68 | A | O |
| ATOM | 311 | N | GLU | A | 56 | 56.980 | 56.233 | 86.492 | 1.00 | 56.91 | A | N |
| ATOM | 312 | CA | GLU | A | 56 | 55.772 | 55.770 | 85.808 | 1.00 | 54.27 | A | C |
| ATOM | 313 | CB | GLU | A | 56 | 55.704 | 56.378 | 84.395 | 1.00 | 53.86 | A | C |
| ATOM | 314 | CG | GLU | A | 56 | 54.308 | 56.390 | 83.788 | 1.00 | 51.37 | A | C |
| ATOM | 315 | CD | GLU | A | 56 | 53.250 | 56.845 | 84.780 | 1.00 | 51.59 | A | C |
| ATOM | 316 | OE1 | GLU | A | 56 | 53.529 | 57.774 | 85.571 | 1.00 | 54.79 | A | O |
| ATOM | 317 | OE2 | GLU | A | 56 | 52.135 | 56.286 | 84.770 | 1.00 | 50.25 | A | O |
| ATOM | 318 | C | GLU | A | 56 | 55.653 | 54.245 | 85.733 | 1.00 | 50.59 | A | C |
| ATOM | 319 | O | GLU | A | 56 | 54.627 | 53.683 | 86.130 | 1.00 | 47.63 | A | O |
| ATOM | 320 | N | LEU | A | 57 | 56.691 | 53.575 | 85.237 | 1.00 | 47.55 | A | N |
| ATOM | 321 | CA | LEU | A | 57 | 56.654 | 52.118 | 85.143 | 1.00 | 46.68 | A | C |
| ATOM | 322 | CB | LEU | A | 57 | 57.969 | 51.593 | 84.575 | 1.00 | 45.64 | A | C |
| ATOM | 323 | CG | LEU | A | 57 | 58.047 | 51.794 | 83.061 | 1.00 | 46.96 | A | C |
| ATOM | 324 | CD1 | LEU | A | 57 | 59.461 | 51.549 | 82.553 | 1.00 | 46.04 | A | C |
| ATOM | 325 | CD2 | LEU | A | 57 | 57.036 | 50.860 | 82.387 | 1.00 | 47.95 | A | C |
| ATOM | 326 | C | LEU | A | 57 | 56.338 | 51.479 | 86.490 | 1.00 | 46.88 | A | C |
| ATOM | 327 | O | LEU | A | 57 | 55.502 | 50.572 | 86.574 | 1.00 | 48.97 | A | O |
| ATOM | 328 | N | PRO | A | 58 | 57.003 | 51.934 | 87.569 | 1.00 | 47.97 | A | N |
| ATOM | 329 | CD | PRO | A | 58 | 58.269 | 52.690 | 87.584 | 1.00 | 48.07 | A | C |
| ATOM | 330 | CA | PRO | A | 58 | 56.737 | 51.370 | 88.896 | 1.00 | 47.76 | A | C |
| ATOM | 331 | CB | PRO | A | 58 | 57.738 | 52.101 | 89.779 | 1.00 | 47.65 | A | C |
| ATOM | 332 | CG | PRO | A | 58 | 58.909 | 52.216 | 88.876 | 1.00 | 48.78 | A | C |
| ATOM | 333 | C | PRO | A | 58 | 55.293 | 51.607 | 89.323 | 1.00 | 48.43 | A | C |
| ATOM | 334 | O | PRO | A | 58 | 54.657 | 50.718 | 89.912 | 1.00 | 46.87 | A | O |
| ATOM | 335 | N | VAL | A | 59 | 54.776 | 52.801 | 89.024 | 1.00 | 49.09 | A | N |
| ATOM | 336 | CA | VAL | A | 59 | 53.393 | 53.134 | 89.366 | 1.00 | 48.62 | A | C |
| ATOM | 337 | CB | VAL | A | 59 | 53.052 | 54.604 | 89.006 | 1.00 | 51.14 | A | C |
| ATOM | 338 | CG1 | VAL | A | 59 | 51.567 | 54.885 | 89.306 | 1.00 | 50.09 | A | C |
| ATOM | 339 | CG2 | VAL | A | 59 | 53.942 | 55.566 | 89.813 | 1.00 | 51.98 | A | C |
| ATOM | 340 | C | VAL | A | 59 | 52.419 | 52.215 | 88.624 | 1.00 | 48.64 | A | C |
| ATOM | 341 | O | VAL | A | 59 | 51.450 | 51.709 | 89.212 | 1.00 | 50.19 | A | O |
| ATOM | 342 | N | ARG | A | 60 | 52.677 | 51.985 | 87.339 | 1.00 | 46.71 | A | N |
| ATOM | 343 | CA | ARG | A | 60 | 51.798 | 51.128 | 86.533 | 1.00 | 48.80 | A | C |
| ATOM | 344 | CB | ARG | A | 60 | 52.113 | 51.301 | 85.040 | 1.00 | 49.09 | A | C |
| ATOM | 345 | CG | ARG | A | 60 | 51.784 | 52.704 | 84.531 | 1.00 | 49.31 | A | C |
| ATOM | 346 | CD | ARG | A | 60 | 51.825 | 52.804 | 83.019 | 1.00 | 49.09 | A | C |
| ATOM | 347 | NE | ARG | A | 60 | 51.584 | 54.177 | 82.599 | 1.00 | 51.81 | A | N |
| ATOM | 348 | CZ | ARG | A | 60 | 51.448 | 54.582 | 81.339 | 1.00 | 53.00 | A | C |
| ATOM | 349 | NH1 | ARG | A | 60 | 51.516 | 53.720 | 80.330 | 1.00 | 51.51 | A | N |
| ATOM | 350 | NH2 | ARG | A | 60 | 51.273 | 55.872 | 81.090 | 1.00 | 53.60 | A | N |
| ATOM | 351 | C | ARG | A | 60 | 51.905 | 49.660 | 86.937 | 1.00 | 48.33 | A | C |
| ATOM | 352 | O | ARG | A | 60 | 50.896 | 48.954 | 87.055 | 1.00 | 47.50 | A | O |
| ATOM | 353 | N | LEU | A | 61 | 53.129 | 49.195 | 87.162 | 1.00 | 49.41 | A | N |
| ATOM | 354 | CA | LEU | A | 61 | 53.323 | 47.817 | 87.599 | 1.00 | 48.47 | A | C |
| ATOM | 355 | CB | LEU | A | 61 | 54.805 | 47.552 | 87.786 | 1.00 | 47.20 | A | C |
| ATOM | 356 | CG | LEU | A | 61 | 55.624 | 47.551 | 86.504 | 1.00 | 47.38 | A | C |
| ATOM | 357 | CD1 | LEU | A | 61 | 57.100 | 47.545 | 86.854 | 1.00 | 46.38 | A | C |
| ATOM | 358 | CD2 | LEU | A | 61 | 55.246 | 46.328 | 85.662 | 1.00 | 45.57 | A | C |
| ATOM | 359 | C | LEU | A | 61 | 52.597 | 47.599 | 88.931 | 1.00 | 49.62 | A | C |
| ATOM | 360 | O | LEU | A | 61 | 51.876 | 46.602 | 89.122 | 1.00 | 48.67 | A | O |
| ATOM | 361 | N | ALA | A | 62 | 52.775 | 48.555 | 89.839 | 1.00 | 50.34 | A | N |
| ATOM | 362 | CA | ALA | A | 62 | 52.175 | 48.486 | 91.164 | 1.00 | 53.14 | A | C |
| ATOM | 363 | CB | ALA | A | 62 | 52.673 | 49.665 | 92.026 | 1.00 | 52.02 | A | C |
| ATOM | 364 | C | ALA | A | 62 | 50.653 | 48.455 | 91.141 | 1.00 | 54.21 | A | C |
| ATOM | 365 | O | ALA | A | 62 | 50.032 | 47.677 | 91.869 | 1.00 | 54.29 | A | O |
| ATOM | 366 | N | ASN | A | 63 | 50.046 | 49.304 | 90.315 | 1.00 | 56.28 | A | N |
| ATOM | 367 | CA | ASN | A | 63 | 48.589 | 49.342 | 90.252 | 1.00 | 56.77 | A | C |
| ATOM | 368 | CB | ASN | A | 63 | 48.107 | 50.505 | 89.368 | 1.00 | 61.74 | A | C |
| ATOM | 369 | CG | ASN | A | 63 | 48.075 | 51.848 | 90.113 | 1.00 | 64.09 | A | C |
| ATOM | 370 | OD1 | ASN | A | 63 | 49.117 | 52.421 | 90.441 | 1.00 | 65.15 | A | O |
| ATOM | 371 | ND2 | ASN | A | 63 | 46.870 | 52.345 | 90.384 | 1.00 | 64.96 | A | N |
| ATOM | 372 | C | ASN | A | 63 | 48.026 | 48.017 | 89.749 | 1.00 | 54.96 | A | C |
| ATOM | 373 | O | ASN | A | 63 | 47.060 | 47.496 | 90.308 | 1.00 | 54.63 | A | O |
| ATOM | 374 | N | ILE | A | 64 | 48.620 | 47.458 | 88.700 | 1.00 | 53.93 | A | N |
| ATOM | 375 | CA | ILE | A | 64 | 48.124 | 46.179 | 88.211 | 1.00 | 57.40 | A | C |
| ATOM | 376 | CB | ILE | A | 64 | 48.727 | 45.809 | 86.824 | 1.00 | 57.13 | A | C |
| ATOM | 377 | CG2 | ILE | A | 64 | 50.189 | 45.445 | 86.943 | 1.00 | 53.77 | A | C |
| ATOM | 378 | CG1 | ILE | A | 64 | 47.954 | 44.629 | 86.239 | 1.00 | 58.20 | A | C |
| ATOM | 379 | CD1 | ILE | A | 64 | 46.439 | 44.825 | 86.237 | 1.00 | 58.25 | A | C |
| ATOM | 380 | C | ILE | A | 64 | 48.415 | 45.062 | 89.236 | 1.00 | 59.85 | A | C |
| ATOM | 381 | O | ILE | A | 64 | 47.582 | 44.171 | 89.451 | 1.00 | 58.38 | A | O |
| ATOM | 382 | N | MET | A | 65 | 49.582 | 45.124 | 89.880 | 1.00 | 62.55 | A | N |
| ATOM | 383 | CA | MET | A | 65 | 49.949 | 44.130 | 90.890 | 1.00 | 66.24 | A | C |
| ATOM | 384 | CB | MET | A | 65 | 51.405 | 44.311 | 91.324 | 1.00 | 63.68 | A | C |
| ATOM | 385 | CG | MET | A | 65 | 52.408 | 43.820 | 90.316 | 1.00 | 63.75 | A | C |
| ATOM | 386 | SD | MET | A | 65 | 54.061 | 44.343 | 90.743 | 1.00 | 67.20 | A | S |
| ATOM | 387 | CE | MET | A | 65 | 54.491 | 43.040 | 91.910 | 1.00 | 63.16 | A | C |
| ATOM | 388 | C | MET | A | 65 | 49.053 | 44.239 | 92.115 | 1.00 | 70.05 | A | C |
| ATOM | 389 | O | MET | A | 65 | 48.684 | 43.228 | 92.715 | 1.00 | 69.49 | A | O |
| ATOM | 390 | N | LYS | A | 66 | 48.709 | 45.473 | 92.480 | 1.00 | 74.83 | A | N |
| ATOM | 391 | CA | LYS | A | 66 | 47.872 | 45.728 | 93.647 | 1.00 | 80.23 | A | C |
| ATOM | 392 | CB | LYS | A | 66 | 47.814 | 47.236 | 93.921 | 1.00 | 80.04 | A | C |
| ATOM | 393 | CG | LYS | A | 66 | 47.881 | 47.635 | 95.398 | 1.00 | 80.04 | A | C |
| ATOM | 394 | CD | LYS | A | 66 | 46.630 | 47.229 | 96.157 | 1.00 | 81.12 | A | C |
| ATOM | 395 | CE | LYS | A | 66 | 46.683 | 47.707 | 97.599 | 1.00 | 81.22 | A | C |
| ATOM | 396 | NZ | LYS | A | 66 | 45.454 | 47.334 | 98.358 | 1.00 | 82.72 | A | N |
| ATOM | 397 | C | LYS | A | 66 | 46.461 | 45.174 | 93.447 | 1.00 | 83.78 | A | C |
| ATOM | 398 | O | LYS | A | 66 | 45.815 | 44.740 | 94.403 | 1.00 | 84.69 | A | O |
| ATOM | 399 | N | GLU | A | 67 | 45.987 | 45.185 | 92.205 | 1.00 | 87.85 | A | N |
| ATOM | 400 | CA | GLU | A | 67 | 44.650 | 44.690 | 91.906 | 1.00 | 92.52 | A | C |
| ATOM | 401 | CB | GLU | A | 67 | 44.180 | 45.226 | 90.558 | 1.00 | 95.72 | A | C |
| ATOM | 402 | CG | GLU | A | 67 | 42.741 | 44.873 | 90.231 | 1.00 | 101.58 | A | C |
| ATOM | 403 | CD | GLU | A | 67 | 42.178 | 45.710 | 89.098 | 1.00 | 103.89 | A | C |
| ATOM | 404 | OE1 | GLU | A | 67 | 42.087 | 46.946 | 89.260 | 1.00 | 105.19 | A | O |
| ATOM | 405 | OE2 | GLU | A | 67 | 41.827 | 45.133 | 88.049 | 1.00 | 105.86 | A | O |
| ATOM | 406 | C | GLU | A | 67 | 44.638 | 43.165 | 91.890 | 1.00 | 94.00 | A | C |
| ATOM | 407 | O | GLU | A | 67 | 43.663 | 42.528 | 92.311 | 1.00 | 94.55 | A | O |
| ATOM | 408 | N | ILE | A | 68 | 45.726 | 42.582 | 91.401 | 1.00 | 93.86 | A | N |
| ATOM | 409 | CA | ILE | A | 68 | 45.846 | 41.136 | 91.353 | 1.00 | 93.61 | A | C |
| ATOM | 410 | CB | ILE | A | 68 | 47.178 | 40.710 | 90.688 | 1.00 | 91.17 | A | C |
| ATOM | 411 | CG2 | ILE | A | 68 | 47.341 | 39.206 | 90.764 | 1.00 | 89.83 | A | C |
| ATOM | 412 | CG1 | ILE | A | 68 | 47.203 | 41.173 | 89.229 | 1.00 | 88.01 | A | C |
| ATOM | 413 | CD1 | ILE | A | 68 | 48.463 | 40.794 | 88.487 | 1.00 | 83.32 | A | C |
| ATOM | 414 | C | ILE | A | 68 | 45.783 | 40.590 | 92.780 | 1.00 | 95.62 | A | C |
| ATOM | 415 | O | ILE | A | 68 | 44.953 | 39.740 | 93.088 | 1.00 | 96.13 | A | O |
| ATOM | 416 | N | ASN | A | 69 | 46.653 | 41.096 | 93.651 | 1.00 | 97.21 | A | N |
| ATOM | 417 | CA | ASN | A | 69 | 46.692 | 40.651 | 95.042 | 1.00 | 99.00 | A | C |
| ATOM | 418 | CB | ASN | A | 69 | 47.637 | 41.524 | 95.880 | 1.00 | 99.22 | A | C |
| ATOM | 419 | CG | ASN | A | 69 | 49.099 | 41.316 | 95.533 | 1.00 | 99.37 | A | C |
| ATOM | 420 | OD1 | ASN | A | 69 | 49.520 | 40.211 | 95.193 | 1.00 | 98.65 | A | O |
| ATOM | 421 | ND2 | ASN | A | 69 | 49.887 | 42.381 | 95.639 | 1.00 | 99.85 | A | N |
| ATOM | 422 | C | ASN | A | 69 | 45.327 | 40.655 | 95.716 | 1.00 | 100.06 | A | C |
| ATOM | 423 | O | ASN | A | 69 | 45.144 | 40.016 | 96.752 | 1.00 | 100.53 | A | O |
| ATOM | 424 | N | LEU | A | 70 | 44.366 | 41.369 | 95.138 | 1.00 | 101.35 | A | N |
| ATOM | 425 | CA | LEU | A | 70 | 43.044 | 41.446 | 95.742 | 1.00 | 102.96 | A | C |
| ATOM | 426 | CB | LEU | A | 70 | 42.622 | 42.913 | 95.884 | 1.00 | 106.14 | A | C |
| ATOM | 427 | CG | LEU | A | 70 | 43.472 | 43.739 | 96.859 | 1.00 | 108.62 | A | C |
| ATOM | 428 | CD1 | LEU | A | 70 | 42.896 | 45.140 | 96.967 | 1.00 | 109.58 | A | C |
| ATOM | 429 | CD2 | LEU | A | 70 | 43.497 | 43.070 | 98.234 | 1.00 | 109.35 | A | C |
| ATOM | 430 | C | LEU | A | 70 | 41.933 | 40.643 | 95.069 | 1.00 | 102.00 | A | C |
| ATOM | 431 | O | LEU | A | 70 | 40.764 | 41.024 | 95.121 | 1.00 | 102.53 | A | O |
| ATOM | 432 | N | LEU | A | 71 | 42.297 | 39.534 | 94.436 | 1.00 | 100.25 | A | N |
| ATOM | 433 | CA | LEU | A | 71 | 41.309 | 38.665 | 93.809 | 1.00 | 98.06 | A | C |
| ATOM | 434 | CB | LEU | A | 71 | 41.638 | 38.448 | 92.321 | 1.00 | 97.35 | A | C |
| ATOM | 435 | CG | LEU | A | 71 | 43.067 | 38.182 | 91.842 | 1.00 | 96.14 | A | C |
| ATOM | 436 | CD1 | LEU | A | 71 | 43.501 | 36.785 | 92.235 | 1.00 | 96.35 | A | C |
| ATOM | 437 | CD2 | LEU | A | 71 | 43.129 | 38.342 | 90.332 | 1.00 | 94.73 | A | C |
| ATOM | 438 | C | LEU | A | 71 | 41.329 | 37.353 | 94.601 | 1.00 | 96.54 | A | C |
| ATOM | 439 | O | LEU | A | 71 | 42.293 | 37.077 | 95.318 | 1.00 | 96.07 | A | O |
| ATOM | 440 | N | PRO | A | 72 | 40.263 | 36.537 | 94.500 | 1.00 | 95.04 | A | N |
| ATOM | 441 | CD | PRO | A | 72 | 39.158 | 36.629 | 93.528 | 1.00 | 93.95 | A | C |
| ATOM | 442 | CA | PRO | A | 72 | 40.198 | 35.266 | 95.234 | 1.00 | 94.67 | A | C |
| ATOM | 443 | CB | PRO | A | 72 | 39.148 | 34.479 | 94.455 | 1.00 | 93.93 | A | C |
| ATOM | 444 | CG | PRO | A | 72 | 38.215 | 35.548 | 94.008 | 1.00 | 93.60 | A | C |
| ATOM | 445 | C | PRO | A | 72 | 41.532 | 34.518 | 95.339 | 1.00 | 94.99 | A | C |
| ATOM | 446 | O | PRO | A | 72 | 42.289 | 34.449 | 94.369 | 1.00 | 94.40 | A | O |
| ATOM | 447 | N | ASP | A | 73 | 41.819 | 33.970 | 96.520 | 1.00 | 95.44 | A | N |
| ATOM | 448 | CA | ASP | A | 73 | 43.062 | 33.228 | 96.730 | 1.00 | 95.90 | A | C |
| ATOM | 449 | CB | ASP | A | 73 | 43.226 | 32.846 | 98.204 | 1.00 | 97.89 | A | C |
| ATOM | 450 | CG | ASP | A | 73 | 44.525 | 32.098 | 98.474 | 1.00 | 99.42 | A | C |
| ATOM | 451 | OD1 | ASP | A | 73 | 45.611 | 32.655 | 98.189 | 1.00 | 99.28 | A | O |
| ATOM | 452 | OD2 | ASP | A | 73 | 44.458 | 30.953 | 98.974 | 1.00 | 99.41 | A | O |
| ATOM | 453 | C | ASP | A | 73 | 43.029 | 31.970 | 95.874 | 1.00 | 94.78 | A | C |
| ATOM | 454 | O | ASP | A | 73 | 44.054 | 31.331 | 95.630 | 1.00 | 94.08 | A | O |
| ATOM | 455 | N | ARG | A | 74 | 41.829 | 31.626 | 95.424 | 1.00 | 93.58 | A | N |
| ATOM | 456 | CA | ARG | A | 74 | 41.626 | 30.461 | 94.579 | 1.00 | 92.05 | A | C |
| ATOM | 457 | CB | ARG | A | 74 | 40.137 | 30.276 | 94.322 | 1.00 | 91.83 | A | C |
| ATOM | 458 | CG | ARG | A | 74 | 39.275 | 30.529 | 95.544 | 1.00 | 92.12 | A | C |
| ATOM | 459 | CD | ARG | A | 74 | 38.087 | 31.362 | 95.142 | 1.00 | 91.27 | A | C |
| ATOM | 460 | NE | ARG | A | 74 | 37.615 | 30.957 | 93.823 | 1.00 | 88.83 | A | N |
| ATOM | 461 | CZ | ARG | A | 74 | 36.550 | 31.468 | 93.219 | 1.00 | 88.43 | A | C |
| ATOM | 462 | NH1 | ARG | A | 74 | 35.832 | 32.411 | 93.814 | 1.00 | 87.60 | A | N |
| ATOM | 463 | NH2 | ARG | A | 74 | 36.206 | 31.033 | 92.017 | 1.00 | 87.62 | A | N |
| ATOM | 464 | C | ARG | A | 74 | 42.347 | 30.725 | 93.261 | 1.00 | 90.81 | A | C |
| ATOM | 465 | O | ARG | A | 74 | 42.973 | 29.835 | 92.684 | 1.00 | 91.35 | A | O |
| ATOM | 466 | N | VAL | A | 75 | 42.254 | 31.964 | 92.792 | 1.00 | 88.53 | A | N |
| ATOM | 467 | CA | VAL | A | 75 | 42.901 | 32.362 | 91.552 | 1.00 | 86.11 | A | C |
| ATOM | 468 | CB | VAL | A | 75 | 42.166 | 33.555 | 90.906 | 1.00 | 83.66 | A | C |
| ATOM | 469 | CG1 | VAL | A | 75 | 42.923 | 34.032 | 89.683 | 1.00 | 82.62 | A | C |
| ATOM | 470 | CG2 | VAL | A | 75 | 40.755 | 33.139 | 90.522 | 1.00 | 81.64 | A | C |
| ATOM | 471 | C | VAL | A | 75 | 44.360 | 32.734 | 91.806 | 1.00 | 85.73 | A | C |
| ATOM | 472 | O | VAL | A | 75 | 45.224 | 32.494 | 90.968 | 1.00 | 85.98 | A | O |
| ATOM | 473 | N | LEU | A | 76 | 44.630 | 33.309 | 92.971 | 1.00 | 85.73 | A | N |
| ATOM | 474 | CA | LEU | A | 76 | 45.989 | 33.705 | 93.332 | 1.00 | 87.32 | A | C |
| ATOM | 475 | CB | LEU | A | 76 | 45.983 | 34.448 | 94.672 | 1.00 | 87.55 | A | C |
| ATOM | 476 | CG | LEU | A | 76 | 45.442 | 35.871 | 94.740 | 1.00 | 86.72 | A | C |
| ATOM | 477 | CD1 | LEU | A | 76 | 45.338 | 36.307 | 96.190 | 1.00 | 87.04 | A | C |
| ATOM | 478 | CD2 | LEU | A | 76 | 46.360 | 36.794 | 93.966 | 1.00 | 86.77 | A | C |
| ATOM | 479 | C | LEU | A | 76 | 46.944 | 32.516 | 93.444 | 1.00 | 87.71 | A | C |
| ATOM | 480 | O | LEU | A | 76 | 48.090 | 32.582 | 92.991 | 1.00 | 86.76 | A | O |
| ATOM | 481 | N | SER | A | 77 | 46.459 | 31.436 | 94.054 | 1.00 | 88.19 | A | N |
| ATOM | 482 | CA | SER | A | 77 | 47.257 | 30.232 | 94.280 | 1.00 | 88.86 | A | C |
| ATOM | 483 | CB | SER | A | 77 | 46.560 | 29.344 | 95.313 | 1.00 | 90.01 | A | C |
| ATOM | 484 | OG | SER | A | 77 | 46.576 | 29.959 | 96.591 | 1.00 | 91.93 | A | O |
| ATOM | 485 | C | SER | A | 77 | 47.637 | 29.383 | 93.068 | 1.00 | 87.88 | A | C |
| ATOM | 486 | O | SER | A | 77 | 48.493 | 28.504 | 93.182 | 1.00 | 88.39 | A | O |
| ATOM | 487 | N | THR | A | 78 | 47.012 | 29.631 | 91.919 | 1.00 | 86.13 | A | N |
| ATOM | 488 | CA | THR | A | 78 | 47.325 | 28.868 | 90.711 | 1.00 | 82.82 | A | C |
| ATOM | 489 | CB | THR | A | 78 | 46.304 | 29.131 | 89.593 | 1.00 | 82.51 | A | C |
| ATOM | 490 | OG1 | THR | A | 78 | 46.208 | 30.539 | 89.351 | 1.00 | 83.68 | A | O |
| ATOM | 491 | CG2 | THR | A | 78 | 44.944 | 28.604 | 89.987 | 1.00 | 82.48 | A | C |
| ATOM | 492 | C | THR | A | 78 | 48.714 | 29.231 | 90.198 | 1.00 | 80.18 | A | C |
| ATOM | 493 | O | THR | A | 78 | 49.101 | 30.399 | 90.196 | 1.00 | 80.10 | A | O |
| ATOM | 494 | N | PRO | A | 79 | 49.485 | 28.225 | 89.760 | 1.00 | 77.67 | A | N |
| ATOM | 495 | CD | PRO | A | 79 | 49.092 | 26.809 | 89.643 | 1.00 | 76.28 | A | C |
| ATOM | 496 | CA | PRO | A | 79 | 50.843 | 28.429 | 89.243 | 1.00 | 74.44 | A | C |
| ATOM | 497 | CB | PRO | A | 79 | 51.178 | 27.074 | 88.619 | 1.00 | 74.46 | A | C |
| ATOM | 498 | CG | PRO | A | 79 | 50.432 | 26.119 | 89.490 | 1.00 | 75.08 | A | C |
| ATOM | 499 | C | PRO | A | 79 | 50.914 | 29.565 | 88.228 | 1.00 | 71.45 | A | C |
| ATOM | 500 | O | PRO | A | 79 | 51.874 | 30.333 | 88.202 | 1.00 | 70.95 | A | O |
| ATOM | 501 | N | SER | A | 80 | 49.886 | 29.655 | 87.390 | 1.00 | 67.66 | A | N |
| ATOM | 502 | CA | SER | A | 80 | 49.804 | 30.682 | 86.349 | 1.00 | 63.75 | A | C |
| ATOM | 503 | CB | SER | A | 80 | 48.480 | 30.534 | 85.588 | 1.00 | 62.90 | A | C |
| ATOM | 504 | OG | SER | A | 80 | 47.378 | 30.786 | 86.460 | 1.00 | 65.81 | A | O |
| ATOM | 505 | C | SER | A | 80 | 49.889 | 32.088 | 86.940 | 1.00 | 60.15 | A | C |
| ATOM | 506 | O | SER | A | 80 | 50.879 | 32.804 | 86.756 | 1.00 | 57.11 | A | O |
| ATOM | 507 | N | VAL | A | 81 | 48.835 | 32.471 | 87.651 | 1.00 | 59.18 | A | N |
| ATOM | 508 | CA | VAL | A | 81 | 48.763 | 33.781 | 88.269 | 1.00 | 60.09 | A | C |
| ATOM | 509 | CB | VAL | A | 81 | 47.537 | 33.881 | 89.176 | 1.00 | 59.62 | A | C |
| ATOM | 510 | CG1 | VAL | A | 81 | 47.533 | 35.221 | 89.904 | 1.00 | 61.65 | A | C |
| ATOM | 511 | CG2 | VAL | A | 81 | 46.268 | 33.719 | 88.335 | 1.00 | 58.12 | A | C |
| ATOM | 512 | C | VAL | A | 81 | 50.022 | 34.083 | 89.069 | 1.00 | 61.37 | A | C |
| ATOM | 513 | O | VAL | A | 81 | 50.651 | 35.127 | 88.863 | 1.00 | 61.61 | A | O |
| ATOM | 514 | N | GLN | A | 82 | 50.400 | 33.176 | 89.967 | 1.00 | 61.34 | A | N |
| ATOM | 515 | CA | GLN | A | 82 | 51.597 | 33.392 | 90.764 | 1.00 | 63.00 | A | C |
| ATOM | 516 | CB | GLN | A | 82 | 51.798 | 32.260 | 91.783 | 1.00 | 69.00 | A | C |
| ATOM | 517 | CG | GLN | A | 82 | 50.979 | 32.424 | 93.069 | 1.00 | 75.84 | A | C |
| ATOM | 518 | CD | GLN | A | 82 | 51.735 | 31.970 | 94.315 | 1.00 | 80.17 | A | C |
| ATOM | 519 | OE1 | GLN | A | 82 | 52.787 | 32.520 | 94.654 | 1.00 | 81.81 | A | O |
| ATOM | 520 | NE2 | GLN | A | 82 | 51.198 | 30.966 | 95.003 | 1.00 | 82.17 | A | N |
| ATOM | 521 | C | GLN | A | 82 | 52.834 | 33.523 | 89.888 | 1.00 | 61.34 | A | C |
| ATOM | 522 | O | GLN | A | 82 | 53.713 | 34.336 | 90.172 | 1.00 | 62.49 | A | O |
| ATOM | 523 | N | LEU | A | 83 | 52.906 | 32.735 | 88.821 | 1.00 | 59.54 | A | N |
| ATOM | 524 | CA | LEU | A | 83 | 54.063 | 32.789 | 87.929 | 1.00 | 59.20 | A | C |
| ATOM | 525 | CB | LEU | A | 83 | 53.907 | 31.789 | 86.780 | 1.00 | 58.06 | A | C |
| ATOM | 526 | CG | LEU | A | 83 | 55.165 | 31.275 | 86.065 | 1.00 | 58.37 | A | C |
| ATOM | 527 | CD1 | LEU | A | 83 | 54.752 | 30.716 | 84.711 | 1.00 | 58.09 | A | C |
| ATOM | 528 | CD2 | LEU | A | 83 | 56.193 | 32.376 | 85.863 | 1.00 | 60.62 | A | C |
| ATOM | 529 | C | LEU | A | 83 | 54.248 | 34.191 | 87.340 | 1.00 | 61.00 | A | C |
| ATOM | 530 | O | LEU | A | 83 | 55.360 | 34.733 | 87.357 | 1.00 | 60.51 | A | O |
| ATOM | 531 | N | VAL | A | 84 | 53.171 | 34.772 | 86.806 | 1.00 | 61.95 | A | N |
| ATOM | 532 | CA | VAL | A | 84 | 53.271 | 36.107 | 86.210 | 1.00 | 62.46 | A | C |
| ATOM | 533 | CB | VAL | A | 84 | 52.061 | 36.426 | 85.283 | 1.00 | 59.78 | A | C |
| ATOM | 534 | CG1 | VAL | A | 84 | 51.784 | 35.236 | 84.382 | 1.00 | 57.15 | A | C |
| ATOM | 535 | CG2 | VAL | A | 84 | 50.832 | 36.795 | 86.098 | 1.00 | 60.08 | A | C |
| ATOM | 536 | C | VAL | A | 84 | 53.365 | 37.136 | 87.324 | 1.00 | 62.45 | A | C |
| ATOM | 537 | O | VAL | A | 84 | 54.070 | 38.134 | 87.196 | 1.00 | 61.27 | A | O |
| ATOM | 538 | N | GLN | A | 85 | 52.660 | 36.870 | 88.419 | 1.00 | 64.89 | A | N |
| ATOM | 539 | CA | GLN | A | 85 | 52.677 | 37.749 | 89.580 | 1.00 | 67.84 | A | C |
| ATOM | 540 | CB | GLN | A | 85 | 51.815 | 37.152 | 90.703 | 1.00 | 71.62 | A | C |
| ATOM | 541 | CG | GLN | A | 85 | 51.840 | 37.911 | 92.028 | 1.00 | 80.18 | A | C |
| ATOM | 542 | CD | GLN | A | 85 | 51.233 | 39.300 | 91.930 | 1.00 | 87.00 | A | C |
| ATOM | 543 | OE1 | GLN | A | 85 | 51.755 | 40.174 | 91.230 | 1.00 | 89.23 | A | O |
| ATOM | 544 | NE2 | GLN | A | 85 | 50.121 | 39.510 | 92.633 | 1.00 | 88.68 | A | N |
| ATOM | 545 | C | GLN | A | 85 | 54.125 | 37.897 | 90.044 | 1.00 | 67.21 | A | C |
| ATOM | 546 | O | GLN | A | 85 | 54.513 | 38.948 | 90.559 | 1.00 | 68.55 | A | O |
| ATOM | 547 | N | SER | A | 86 | 54.925 | 36.849 | 89.841 | 1.00 | 64.83 | A | N |
| ATOM | 548 | CA | SER | A | 86 | 56.326 | 36.880 | 90.251 | 1.00 | 65.15 | A | C |
| ATOM | 549 | CB | SER | A | 86 | 56.862 | 35.456 | 90.500 | 1.00 | 65.27 | A | C |
| ATOM | 550 | OG | SER | A | 86 | 57.345 | 34.845 | 89.314 | 1.00 | 63.78 | A | O |
| ATOM | 551 | C | SER | A | 86 | 57.176 | 37.581 | 89.202 | 1.00 | 65.35 | A | C |
| ATOM | 552 | O | SER | A | 86 | 58.269 | 38.070 | 89.493 | 1.00 | 65.05 | A | O |
| ATOM | 553 | N | TRP | A | 87 | 56.681 | 37.622 | 87.973 | 1.00 | 67.66 | A | N |
| ATOM | 554 | CA | TRP | A | 87 | 57.410 | 38.299 | 86.912 | 1.00 | 69.17 | A | C |
| ATOM | 555 | CB | TRP | A | 87 | 56.804 | 37.989 | 85.544 | 1.00 | 76.07 | A | C |
| ATOM | 556 | CG | TRP | A | 87 | 57.180 | 36.668 | 84.991 | 1.00 | 81.36 | A | C |
| ATOM | 557 | CD2 | TRP | A | 87 | 56.477 | 35.929 | 83.982 | 1.00 | 85.80 | A | C |
| ATOM | 558 | CE2 | TRP | A | 87 | 57.228 | 34.762 | 83.723 | 1.00 | 86.80 | A | C |
| ATOM | 559 | CE3 | TRP | A | 87 | 55.288 | 36.142 | 83.268 | 1.00 | 86.75 | A | C |
| ATOM | 560 | CD1 | TRP | A | 87 | 58.290 | 35.942 | 85.294 | 1.00 | 83.80 | A | C |
| ATOM | 561 | NE1 | TRP | A | 87 | 58.329 | 34.794 | 84.539 | 1.00 | 86.21 | A | N |
| ATOM | 562 | CZ2 | TRP | A | 87 | 56.829 | 33.804 | 82.781 | 1.00 | 87.19 | A | C |
| ATOM | 563 | CZ3 | TRP | A | 87 | 54.890 | 35.189 | 82.329 | 1.00 | 87.60 | A | C |
| ATOM | 564 | CH2 | TRP | A | 87 | 55.661 | 34.036 | 82.095 | 1.00 | 88.05 | A | C |
| ATOM | 565 | C | TRP | A | 87 | 57.334 | 39.800 | 87.151 | 1.00 | 65.70 | A | C |
| ATOM | 566 | O | TRP | A | 87 | 58.322 | 40.518 | 87.008 | 1.00 | 62.45 | A | O |
| ATOM | 567 | N | TYR | A | 88 | 56.143 | 40.266 | 87.500 | 1.00 | 63.41 | A | N |
| ATOM | 568 | CA | TYR | A | 88 | 55.945 | 41.678 | 87.760 | 1.00 | 63.04 | A | C |
| ATOM | 569 | CB | TYR | A | 88 | 54.460 | 41.977 | 87.987 | 1.00 | 64.63 | A | C |
| ATOM | 570 | CG | TYR | A | 88 | 53.691 | 42.027 | 86.688 | 1.00 | 68.73 | A | C |
| ATOM | 571 | CD1 | TYR | A | 88 | 53.617 | 40.907 | 85.861 | 1.00 | 69.82 | A | C |
| ATOM | 572 | CE1 | TYR | A | 88 | 53.002 | 40.968 | 84.615 | 1.00 | 71.81 | A | C |
| ATOM | 573 | CD2 | TYR | A | 88 | 53.116 | 43.216 | 86.238 | 1.00 | 69.35 | A | C |
| ATOM | 574 | CE2 | TYR | A | 88 | 52.499 | 43.286 | 84.990 | 1.00 | 71.77 | A | C |
| ATOM | 575 | CZ | TYR | A | 88 | 52.450 | 42.159 | 84.183 | 1.00 | 71.19 | A | C |
| ATOM | 576 | OH | TYR | A | 88 | 51.886 | 42.223 | 82.932 | 1.00 | 72.63 | A | O |
| ATOM | 577 | C | TYR | A | 88 | 56.773 | 42.097 | 88.959 | 1.00 | 61.97 | A | C |
| ATOM | 578 | O | TYR | A | 88 | 57.438 | 43.136 | 88.917 | 1.00 | 60.45 | A | O |
| ATOM | 579 | N | VAL | A | 89 | 56.757 | 41.274 | 90.011 | 1.00 | 60.64 | A | N |
| ATOM | 580 | CA | VAL | A | 89 | 57.522 | 41.559 | 91.224 | 1.00 | 57.25 | A | C |
| ATOM | 581 | CB | VAL | A | 89 | 57.388 | 40.403 | 92.256 | 1.00 | 58.03 | A | C |
| ATOM | 582 | CG1 | VAL | A | 89 | 58.350 | 40.623 | 93.426 | 1.00 | 57.54 | A | C |
| ATOM | 583 | CG2 | VAL | A | 89 | 55.938 | 40.322 | 92.778 | 1.00 | 53.58 | A | C |
| ATOM | 584 | C | VAL | A | 89 | 58.985 | 41.758 | 90.841 | 1.00 | 57.64 | A | C |
| ATOM | 585 | O | VAL | A | 89 | 59.635 | 42.713 | 91.276 | 1.00 | 55.60 | A | O |
| ATOM | 586 | N | GLN | A | 90 | 59.496 | 40.868 | 90.000 | 1.00 | 58.53 | A | N |
| ATOM | 587 | CA | GLN | A | 90 | 60.880 | 40.969 | 89.566 | 1.00 | 61.08 | A | C |
| ATOM | 588 | CB | GLN | A | 90 | 61.305 | 39.707 | 88.810 | 1.00 | 64.26 | A | C |
| ATOM | 589 | CG | GLN | A | 90 | 61.868 | 38.631 | 89.718 | 1.00 | 72.24 | A | C |
| ATOM | 590 | CD | GLN | A | 90 | 63.007 | 39.155 | 90.569 | 1.00 | 74.11 | A | C |
| ATOM | 591 | OE1 | GLN | A | 90 | 62.880 | 39.273 | 91.788 | 1.00 | 77.03 | A | O |
| ATOM | 592 | NE2 | GLN | A | 90 | 64.123 | 39.489 | 89.925 | 1.00 | 74.48 | A | N |
| ATOM | 593 | C | GLN | A | 90 | 61.122 | 42.187 | 88.695 | 1.00 | 61.47 | A | C |
| ATOM | 594 | O | GLN | A | 90 | 62.183 | 42.822 | 88.782 | 1.00 | 60.85 | A | O |
| ATOM | 595 | N | SER | A | 91 | 60.153 | 42.511 | 87.840 | 1.00 | 61.78 | A | N |
| ATOM | 596 | CA | SER | A | 91 | 60.300 | 43.671 | 86.969 | 1.00 | 61.11 | A | C |
| ATOM | 597 | CB | SER | A | 91 | 59.209 | 43.681 | 85.890 | 1.00 | 62.90 | A | C |
| ATOM | 598 | OG | SER | A | 91 | 59.555 | 42.812 | 84.825 | 1.00 | 63.59 | A | O |
| ATOM | 599 | C | SER | A | 91 | 60.259 | 44.955 | 87.790 | 1.00 | 58.77 | A | C |
| ATOM | 600 | O | SER | A | 91 | 60.999 | 45.893 | 87.510 | 1.00 | 57.23 | A | O |
| ATOM | 601 | N | LEU | A | 92 | 59.401 | 44.987 | 88.805 | 1.00 | 58.86 | A | N |
| ATOM | 602 | CA | LEU | A | 92 | 59.291 | 46.153 | 89.677 | 1.00 | 60.91 | A | C |
| ATOM | 603 | CB | LEU | A | 92 | 58.192 | 45.934 | 90.731 | 1.00 | 58.63 | A | C |
| ATOM | 604 | CG | LEU | A | 92 | 57.972 | 47.030 | 91.788 | 1.00 | 59.92 | A | C |
| ATOM | 605 | CD1 | LEU | A | 92 | 57.666 | 48.349 | 91.101 | 1.00 | 59.80 | A | C |
| ATOM | 606 | CD2 | LEU | A | 92 | 56.831 | 46.648 | 92.723 | 1.00 | 56.83 | A | C |
| ATOM | 607 | C | LEU | A | 92 | 60.644 | 46.370 | 90.359 | 1.00 | 63.59 | A | C |
| ATOM | 608 | O | LEU | A | 92 | 61.171 | 47.483 | 90.365 | 1.00 | 64.64 | A | O |
| ATOM | 609 | N | LEU | A | 93 | 61.215 | 45.296 | 90.908 | 1.00 | 66.48 | A | N |
| ATOM | 610 | CA | LEU | A | 93 | 62.505 | 45.367 | 91.594 | 1.00 | 67.70 | A | C |
| ATOM | 611 | CB | LEU | A | 93 | 62.813 | 44.034 | 92.271 | 1.00 | 64.74 | A | C |
| ATOM | 612 | CG | LEU | A | 93 | 61.746 | 43.462 | 93.209 | 1.00 | 64.88 | A | C |
| ATOM | 613 | CD1 | LEU | A | 93 | 62.205 | 42.095 | 93.701 | 1.00 | 62.21 | A | C |
| ATOM | 614 | CD2 | LEU | A | 93 | 61.493 | 44.407 | 94.378 | 1.00 | 62.90 | A | C |
| ATOM | 615 | C | LEU | A | 93 | 63.617 | 45.705 | 90.606 | 1.00 | 70.03 | A | C |
| ATOM | 616 | O | LEU | A | 93 | 64.567 | 46.420 | 90.935 | 1.00 | 71.92 | A | O |
| ATOM | 617 | N | ASP | A | 94 | 63.488 | 45.180 | 89.393 | 1.00 | 71.96 | A | N |
| ATOM | 618 | CA | ASP | A | 94 | 64.454 | 45.413 | 88.327 | 1.00 | 74.63 | A | C |
| ATOM | 619 | CB | ASP | A | 94 | 63.998 | 44.703 | 87.054 | 1.00 | 78.80 | A | C |
| ATOM | 620 | CG | ASP | A | 94 | 64.612 | 43.337 | 86.892 | 1.00 | 81.24 | A | C |
| ATOM | 621 | OD1 | ASP | A | 94 | 64.567 | 42.543 | 87.856 | 1.00 | 82.33 | A | O |
| ATOM | 622 | OD2 | ASP | A | 94 | 65.131 | 43.060 | 85.787 | 1.00 | 82.64 | A | O |
| ATOM | 623 | C | ASP | A | 94 | 64.635 | 46.894 | 88.007 | 1.00 | 75.67 | A | C |
| ATOM | 624 | O | ASP | A | 94 | 65.698 | 47.312 | 87.536 | 1.00 | 75.87 | A | O |
| ATOM | 625 | N | ILE | A | 95 | 63.594 | 47.685 | 88.242 | 1.00 | 75.93 | A | N |
| ATOM | 626 | CA | ILE | A | 95 | 63.662 | 49.105 | 87.938 | 1.00 | 77.48 | A | C |
| ATOM | 627 | CB | ILE | A | 95 | 62.353 | 49.593 | 87.262 | 1.00 | 78.40 | A | C |
| ATOM | 628 | CG2 | ILE | A | 95 | 62.546 | 51.002 | 86.702 | 1.00 | 78.68 | A | C |
| ATOM | 629 | CG1 | ILE | A | 95 | 61.987 | 48.656 | 86.105 | 1.00 | 78.14 | A | C |
| ATOM | 630 | CD1 | ILE | A | 95 | 60.719 | 49.035 | 85.377 | 1.00 | 77.99 | A | C |
| ATOM | 631 | C | ILE | A | 95 | 63.961 | 49.962 | 89.164 | 1.00 | 77.29 | A | C |
| ATOM | 632 | O | ILE | A | 95 | 64.448 | 51.085 | 89.035 | 1.00 | 76.29 | A | O |
| ATOM | 633 | N | MET | A | 96 | 63.674 | 49.433 | 90.349 | 1.00 | 78.40 | A | N |
| ATOM | 634 | CA | MET | A | 96 | 63.939 | 50.162 | 91.586 | 1.00 | 80.06 | A | C |
| ATOM | 635 | CB | MET | A | 96 | 63.707 | 49.247 | 92.793 | 1.00 | 78.96 | A | C |
| ATOM | 636 | CG | MET | A | 96 | 62.266 | 48.816 | 92.964 | 1.00 | 75.85 | A | C |
| ATOM | 637 | SD | MET | A | 96 | 61.190 | 50.236 | 93.166 | 1.00 | 75.13 | A | S |
| ATOM | 638 | CE | MET | A | 96 | 59.767 | 49.490 | 93.789 | 1.00 | 72.88 | A | C |
| ATOM | 639 | C | MET | A | 96 | 65.386 | 50.669 | 91.594 | 1.00 | 81.20 | A | C |
| ATOM | 640 | O | MET | A | 96 | 65.650 | 51.845 | 91.855 | 1.00 | 81.93 | A | O |
| ATOM | 641 | N | GLU | A | 97 | 66.311 | 49.764 | 91.291 | 1.00 | 81.61 | A | N |
| ATOM | 642 | CA | GLU | A | 97 | 67.737 | 50.061 | 91.254 | 1.00 | 83.03 | A | C |
| ATOM | 643 | CB | GLU | A | 97 | 68.435 | 49.124 | 90.267 | 1.00 | 85.05 | A | C |
| ATOM | 644 | CG | GLU | A | 97 | 68.050 | 47.657 | 90.408 | 1.00 | 88.64 | A | C |
| ATOM | 645 | CD | GLU | A | 97 | 68.541 | 47.040 | 91.702 | 1.00 | 90.31 | A | C |
| ATOM | 646 | OE1 | GLU | A | 97 | 69.773 | 46.905 | 91.867 | 1.00 | 91.11 | A | O |
| ATOM | 647 | OE2 | GLU | A | 97 | 67.694 | 46.692 | 92.554 | 1.00 | 91.36 | A | O |
| ATOM | 648 | C | GLU | A | 97 | 68.063 | 51.496 | 90.862 | 1.00 | 83.10 | A | C |
| ATOM | 649 | O | GLU | A | 97 | 68.871 | 52.160 | 91.512 | 1.00 | 84.43 | A | O |
| ATOM | 650 | N | PHE | A | 98 | 67.419 | 51.967 | 89.801 | 1.00 | 82.43 | A | N |
| ATOM | 651 | CA | PHE | A | 98 | 67.658 | 53.302 | 89.259 | 1.00 | 81.92 | A | C |
| ATOM | 652 | CB | PHE | A | 98 | 67.199 | 53.333 | 87.800 | 1.00 | 75.24 | A | C |
| ATOM | 653 | CG | PHE | A | 98 | 67.728 | 52.195 | 86.969 | 1.00 | 65.60 | A | C |
| ATOM | 654 | CD1 | PHE | A | 98 | 68.786 | 52.395 | 86.084 | 1.00 | 63.11 | A | C |
| ATOM | 655 | CD2 | PHE | A | 98 | 67.154 | 50.929 | 87.055 | 1.00 | 61.51 | A | C |
| ATOM | 656 | CE1 | PHE | A | 98 | 69.267 | 51.349 | 85.291 | 1.00 | 59.97 | A | C |
| ATOM | 657 | CE2 | PHE | A | 98 | 67.623 | 49.874 | 86.272 | 1.00 | 59.82 | A | C |
| ATOM | 658 | CZ | PHE | A | 98 | 68.683 | 50.084 | 85.384 | 1.00 | 59.58 | A | C |
| ATOM | 659 | C | PHE | A | 98 | 67.035 | 54.492 | 89.992 | 1.00 | 85.70 | A | C |
| ATOM | 660 | O | PHE | A | 98 | 67.262 | 55.639 | 89.605 | 1.00 | 85.48 | A | O |
| ATOM | 661 | N | LEU | A | 99 | 66.257 | 54.238 | 91.039 | 1.00 | 90.29 | A | N |
| ATOM | 662 | CA | LEU | A | 99 | 65.620 | 55.330 | 91.770 | 1.00 | 96.07 | A | C |
| ATOM | 663 | CB | LEU | A | 99 | 64.703 | 54.778 | 92.860 | 1.00 | 96.16 | A | C |
| ATOM | 664 | CG | LEU | A | 99 | 63.356 | 54.251 | 92.365 | 1.00 | 97.80 | A | C |
| ATOM | 665 | CD1 | LEU | A | 99 | 62.592 | 53.650 | 93.532 | 1.00 | 98.01 | A | C |
| ATOM | 666 | CD2 | LEU | A | 99 | 62.558 | 55.384 | 91.723 | 1.00 | 98.11 | A | C |
| ATOM | 667 | C | LEU | A | 99 | 66.590 | 56.337 | 92.378 | 1.00 | 100.01 | A | C |
| ATOM | 668 | O | LEU | A | 99 | 66.306 | 57.536 | 92.403 | 1.00 | 101.41 | A | O |
| ATOM | 669 | N | ASP | A | 100 | 67.728 | 55.858 | 92.869 | 1.00 | 103.89 | A | N |
| ATOM | 670 | CA | ASP | A | 100 | 68.718 | 56.745 | 93.471 | 1.00 | 107.27 | A | C |
| ATOM | 671 | CB | ASP | A | 100 | 69.322 | 56.105 | 94.725 | 1.00 | 109.94 | A | C |
| ATOM | 672 | CG | ASP | A | 100 | 70.081 | 54.827 | 94.422 | 1.00 | 113.18 | A | C |
| ATOM | 673 | OD1 | ASP | A | 100 | 71.045 | 54.874 | 93.627 | 1.00 | 114.16 | A | O |
| ATOM | 674 | OD2 | ASP | A | 100 | 69.714 | 53.772 | 94.982 | 1.00 | 115.02 | A | O |
| ATOM | 675 | C | ASP | A | 100 | 69.826 | 57.074 | 92.477 | 1.00 | 107.63 | A | C |
| ATOM | 676 | O | ASP | A | 100 | 70.653 | 57.949 | 92.726 | 1.00 | 108.32 | A | O |
| ATOM | 677 | N | LYS | A | 101 | 69.839 | 56.369 | 91.350 | 1.00 | 107.91 | A | N |
| ATOM | 678 | CA | LYS | A | 101 | 70.845 | 56.598 | 90.322 | 1.00 | 108.57 | A | C |
| ATOM | 679 | CB | LYS | A | 101 | 70.698 | 55.579 | 89.192 | 1.00 | 107.69 | A | C |
| ATOM | 680 | CG | LYS | A | 101 | 71.499 | 54.309 | 89.397 | 1.00 | 107.02 | A | C |
| ATOM | 681 | CD | LYS | A | 101 | 71.207 | 53.672 | 90.744 | 1.00 | 106.75 | A | C |
| ATOM | 682 | CE | LYS | A | 101 | 72.083 | 52.451 | 90.979 | 1.00 | 106.04 | A | C |
| ATOM | 683 | NZ | LYS | A | 101 | 71.857 | 51.859 | 92.326 | 1.00 | 105.84 | A | N |
| ATOM | 684 | C | LYS | A | 101 | 70.750 | 58.000 | 89.751 | 1.00 | 110.02 | A | C |
| ATOM | 685 | O | LYS | A | 101 | 69.710 | 58.652 | 89.834 | 1.00 | 110.70 | A | O |
| ATOM | 686 | N | ASP | A | 102 | 71.850 | 58.460 | 89.168 | 1.00 | 111.52 | A | N |
| ATOM | 687 | CA | ASP | A | 102 | 71.895 | 59.788 | 88.575 | 1.00 | 112.95 | A | C |
| ATOM | 688 | CB | ASP | A | 102 | 72.976 | 60.646 | 89.245 | 1.00 | 114.48 | A | C |
| ATOM | 689 | CG | ASP | A | 102 | 72.565 | 61.144 | 90.618 | 1.00 | 116.60 | A | C |
| ATOM | 690 | OD1 | ASP | A | 102 | 71.579 | 61.908 | 90.701 | 1.00 | 117.53 | A | O |
| ATOM | 691 | OD2 | ASP | A | 102 | 73.227 | 60.776 | 91.614 | 1.00 | 117.76 | A | O |
| ATOM | 692 | C | ASP | A | 102 | 72.191 | 59.702 | 87.088 | 1.00 | 112.70 | A | C |
| ATOM | 693 | O | ASP | A | 102 | 73.063 | 58.943 | 86.663 | 1.00 | 112.56 | A | O |
| ATOM | 694 | N | PRO | A | 103 | 71.436 | 60.450 | 86.268 | 1.00 | 112.52 | A | N |
| ATOM | 695 | CD | PRO | A | 103 | 70.139 | 61.089 | 86.552 | 1.00 | 111.88 | A | C |
| ATOM | 696 | CA | PRO | A | 103 | 71.692 | 60.414 | 84.829 | 1.00 | 112.49 | A | C |
| ATOM | 697 | CB | PRO | A | 103 | 70.585 | 61.292 | 84.265 | 1.00 | 111.66 | A | C |
| ATOM | 698 | CG | PRO | A | 103 | 69.452 | 61.022 | 85.211 | 1.00 | 111.47 | A | C |
| ATOM | 699 | C | PRO | A | 103 | 73.077 | 61.012 | 84.626 | 1.00 | 113.50 | A | C |
| ATOM | 700 | O | PRO | A | 103 | 73.990 | 60.723 | 85.395 | 1.00 | 113.56 | A | O |
| ATOM | 701 | N | GLU | A | 104 | 73.233 | 61.866 | 83.621 | 1.00 | 114.91 | A | N |
| ATOM | 702 | CA | GLU | A | 104 | 74.532 | 62.475 | 83.358 | 1.00 | 115.98 | A | C |
| ATOM | 703 | CB | GLU | A | 104 | 75.043 | 63.194 | 84.612 | 1.00 | 117.75 | A | C |
| ATOM | 704 | CG | GLU | A | 104 | 74.146 | 64.323 | 85.103 | 1.00 | 120.27 | A | C |
| ATOM | 705 | CD | GLU | A | 104 | 74.078 | 65.477 | 84.122 | 1.00 | 121.54 | A | C |
| ATOM | 706 | OE1 | GLU | A | 104 | 75.142 | 66.048 | 83.801 | 1.00 | 122.83 | A | O |
| ATOM | 707 | OE2 | GLU | A | 104 | 72.963 | 65.813 | 83.672 | 1.00 | 121.86 | A | O |
| ATOM | 708 | C | GLU | A | 104 | 75.516 | 61.378 | 82.952 | 1.00 | 115.88 | A | C |
| ATOM | 709 | O | GLU | A | 104 | 76.247 | 61.514 | 81.973 | 1.00 | 115.44 | A | O |
| ATOM | 710 | N | ASP | A | 105 | 75.521 | 60.290 | 83.718 | 1.00 | 116.28 | A | N |
| ATOM | 711 | CA | ASP | A | 105 | 76.383 | 59.139 | 83.467 | 1.00 | 116.62 | A | C |
| ATOM | 712 | CB | ASP | A | 105 | 76.395 | 58.217 | 84.693 | 1.00 | 117.83 | A | C |
| ATOM | 713 | CG | ASP | A | 105 | 77.360 | 57.054 | 84.547 | 1.00 | 118.86 | A | C |
| ATOM | 714 | OD1 | ASP | A | 105 | 78.574 | 57.300 | 84.396 | 1.00 | 119.63 | A | O |
| ATOM | 715 | OD2 | ASP | A | 105 | 76.905 | 55.891 | 84.588 | 1.00 | 120.08 | A | O |
| ATOM | 716 | C | ASP | A | 105 | 75.843 | 58.387 | 82.253 | 1.00 | 116.19 | A | C |
| ATOM | 717 | O | ASP | A | 105 | 74.873 | 57.636 | 82.356 | 1.00 | 115.81 | A | O |
| ATOM | 718 | N | HIS | A | 106 | 76.479 | 58.605 | 81.106 | 1.00 | 115.81 | A | N |
| ATOM | 719 | CA | HIS | A | 106 | 76.083 | 57.977 | 79.849 | 1.00 | 114.76 | A | C |
| ATOM | 720 | CB | HIS | A | 106 | 77.049 | 58.395 | 78.738 | 1.00 | 116.51 | A | C |
| ATOM | 721 | CG | HIS | A | 106 | 76.760 | 59.748 | 78.164 | 1.00 | 118.38 | A | C |
| ATOM | 722 | CD2 | HIS | A | 106 | 76.887 | 60.229 | 76.906 | 1.00 | 119.00 | A | C |
| ATOM | 723 | ND1 | HIS | A | 106 | 76.286 | 60.796 | 78.927 | 1.00 | 118.84 | A | N |
| ATOM | 724 | CE1 | HIS | A | 106 | 76.130 | 61.860 | 78.161 | 1.00 | 119.01 | A | C |
| ATOM | 725 | NE2 | HIS | A | 106 | 76.488 | 61.544 | 76.929 | 1.00 | 119.35 | A | N |
| ATOM | 726 | C | HIS | A | 106 | 75.983 | 56.459 | 79.909 | 1.00 | 113.37 | A | C |
| ATOM | 727 | O | HIS | A | 106 | 75.701 | 55.812 | 78.902 | 1.00 | 113.13 | A | O |
| ATOM | 728 | N | ARG | A | 107 | 76.220 | 55.891 | 81.086 | 1.00 | 112.03 | A | N |
| ATOM | 729 | CA | ARG | A | 107 | 76.120 | 54.451 | 81.260 | 1.00 | 110.40 | A | C |
| ATOM | 730 | CB | ARG | A | 107 | 77.459 | 53.849 | 81.704 | 1.00 | 114.44 | A | C |
| ATOM | 731 | CG | ARG | A | 107 | 77.363 | 52.352 | 81.933 | 1.00 | 120.63 | A | C |
| ATOM | 732 | CD | ARG | A | 107 | 78.682 | 51.615 | 81.802 | 1.00 | 126.12 | A | C |
| ATOM | 733 | NE | ARG | A | 107 | 78.429 | 50.180 | 81.679 | 1.00 | 131.75 | A | N |
| ATOM | 734 | CZ | ARG | A | 107 | 79.368 | 49.242 | 81.609 | 1.00 | 134.13 | A | C |
| ATOM | 735 | NH1 | ARG | A | 107 | 80.650 | 49.574 | 81.650 | 1.00 | 135.54 | A | N |
| ATOM | 736 | NH2 | ARG | A | 107 | 79.020 | 47.967 | 81.489 | 1.00 | 135.80 | A | N |
| ATOM | 737 | C | ARG | A | 107 | 75.033 | 54.122 | 82.278 | 1.00 | 106.72 | A | C |
| ATOM | 738 | O | ARG | A | 107 | 74.593 | 52.980 | 82.379 | 1.00 | 105.82 | A | O |
| ATOM | 739 | N | THR | A | 108 | 74.602 | 55.129 | 83.029 | 1.00 | 102.99 | A | N |
| ATOM | 740 | CA | THR | A | 108 | 73.552 | 54.936 | 84.022 | 1.00 | 100.07 | A | C |
| ATOM | 741 | CB | THR | A | 108 | 73.437 | 56.151 | 84.985 | 1.00 | 99.86 | A | C |
| ATOM | 742 | OG1 | THR | A | 108 | 74.527 | 56.136 | 85.917 | 1.00 | 100.12 | A | O |
| ATOM | 743 | CG2 | THR | A | 108 | 72.128 | 56.107 | 85.757 | 1.00 | 98.84 | A | C |
| ATOM | 744 | C | THR | A | 108 | 72.216 | 54.750 | 83.311 | 1.00 | 98.67 | A | C |
| ATOM | 745 | O | THR | A | 108 | 71.329 | 54.056 | 83.808 | 1.00 | 98.70 | A | O |
| ATOM | 746 | N | LEU | A | 109 | 72.083 | 55.359 | 82.135 | 1.00 | 96.80 | A | N |
| ATOM | 747 | CA | LEU | A | 109 | 70.840 | 55.269 | 81.380 | 1.00 | 95.30 | A | C |
| ATOM | 748 | CB | LEU | A | 109 | 70.516 | 56.622 | 80.728 | 1.00 | 93.27 | A | C |
| ATOM | 749 | CG | LEU | A | 109 | 71.245 | 57.131 | 79.484 | 1.00 | 91.75 | A | C |
| ATOM | 750 | CD1 | LEU | A | 109 | 71.077 | 58.646 | 79.428 | 1.00 | 90.93 | A | C |
| ATOM | 751 | CD2 | LEU | A | 109 | 72.718 | 56.769 | 79.523 | 1.00 | 92.65 | A | C |
| ATOM | 752 | C | LEU | A | 109 | 70.804 | 54.152 | 80.341 | 1.00 | 95.40 | A | C |
| ATOM | 753 | O | LEU | A | 109 | 69.742 | 53.833 | 79.816 | 1.00 | 95.96 | A | O |
| ATOM | 754 | N | SER | A | 110 | 71.953 | 53.560 | 80.034 | 1.00 | 95.37 | A | N |
| ATOM | 755 | CA | SER | A | 110 | 71.974 | 52.452 | 79.085 | 1.00 | 95.99 | A | C |
| ATOM | 756 | CB | SER | A | 110 | 73.307 | 52.389 | 78.340 | 1.00 | 95.45 | A | C |
| ATOM | 757 | OG | SER | A | 110 | 74.398 | 52.367 | 79.239 | 1.00 | 97.01 | A | O |
| ATOM | 758 | C | SER | A | 110 | 71.756 | 51.187 | 79.908 | 1.00 | 96.93 | A | C |
| ATOM | 759 | O | SER | A | 110 | 71.349 | 50.149 | 79.388 | 1.00 | 96.97 | A | O |
| ATOM | 760 | N | GLN | A | 111 | 72.036 | 51.294 | 81.205 | 1.00 | 98.58 | A | N |
| ATOM | 761 | CA | GLN | A | 111 | 71.837 | 50.192 | 82.145 | 1.00 | 99.12 | A | C |
| ATOM | 762 | CB | GLN | A | 111 | 72.332 | 50.582 | 83.544 | 1.00 | 103.22 | A | C |
| ATOM | 763 | CG | GLN | A | 111 | 73.837 | 50.649 | 83.718 | 1.00 | 108.50 | A | C |
| ATOM | 764 | CD | GLN | A | 111 | 74.450 | 49.298 | 84.001 | 1.00 | 111.84 | A | C |
| ATOM | 765 | OE1 | GLN | A | 111 | 75.668 | 49.172 | 84.115 | 1.00 | 114.72 | A | O |
| ATOM | 766 | NE2 | GLN | A | 111 | 73.608 | 48.275 | 84.121 | 1.00 | 113.51 | A | N |
| ATOM | 767 | C | GLN | A | 111 | 70.328 | 50.006 | 82.194 | 1.00 | 96.49 | A | C |
| ATOM | 768 | O | GLN | A | 111 | 69.815 | 48.922 | 82.473 | 1.00 | 96.05 | A | O |
| ATOM | 769 | N | PHE | A | 112 | 69.634 | 51.104 | 81.912 | 1.00 | 92.96 | A | N |
| ATOM | 770 | CA | PHE | A | 112 | 68.181 | 51.157 | 81.921 | 1.00 | 88.91 | A | C |
| ATOM | 771 | CB | PHE | A | 112 | 67.737 | 52.612 | 82.069 | 1.00 | 85.20 | A | C |
| ATOM | 772 | CG | PHE | A | 112 | 66.296 | 52.777 | 82.444 | 1.00 | 82.00 | A | C |
| ATOM | 773 | CD1 | PHE | A | 112 | 65.812 | 52.256 | 83.637 | 1.00 | 80.06 | A | C |
| ATOM | 774 | CD2 | PHE | A | 112 | 65.426 | 53.473 | 81.613 | 1.00 | 79.30 | A | C |
| ATOM | 775 | CE1 | PHE | A | 112 | 64.483 | 52.430 | 84.002 | 1.00 | 79.48 | A | C |
| ATOM | 776 | CE2 | PHE | A | 112 | 64.097 | 53.653 | 81.969 | 1.00 | 77.70 | A | C |
| ATOM | 777 | CZ | PHE | A | 112 | 63.623 | 53.129 | 83.165 | 1.00 | 77.87 | A | C |
| ATOM | 778 | C | PHE | A | 112 | 67.585 | 50.551 | 80.651 | 1.00 | 88.40 | A | C |
| ATOM | 779 | O | PHE | A | 112 | 66.724 | 49.672 | 80.722 | 1.00 | 87.50 | A | O |
| ATOM | 780 | N | THR | A | 113 | 68.043 | 51.020 | 79.492 | 1.00 | 86.81 | A | N |
| ATOM | 781 | CA | THR | A | 113 | 67.539 | 50.507 | 78.220 | 1.00 | 85.42 | A | C |
| ATOM | 782 | CB | THR | A | 113 | 68.319 | 51.083 | 77.018 | 1.00 | 85.03 | A | C |
| ATOM | 783 | OG1 | THR | A | 113 | 68.144 | 52.504 | 76.975 | 1.00 | 85.10 | A | O |
| ATOM | 784 | CG2 | THR | A | 113 | 67.814 | 50.484 | 75.715 | 1.00 | 83.52 | A | C |
| ATOM | 785 | C | THR | A | 113 | 67.643 | 48.986 | 78.193 | 1.00 | 84.87 | A | C |
| ATOM | 786 | O | THR | A | 113 | 66.745 | 48.305 | 77.704 | 1.00 | 84.63 | A | O |
| ATOM | 787 | N | ASP | A | 114 | 68.742 | 48.458 | 78.722 | 1.00 | 83.67 | A | N |
| ATOM | 788 | CA | ASP | A | 114 | 68.940 | 47.018 | 78.767 | 1.00 | 81.99 | A | C |
| ATOM | 789 | CB | ASP | A | 114 | 70.387 | 46.684 | 79.141 | 1.00 | 83.34 | A | C |
| ATOM | 790 | CG | ASP | A | 114 | 71.360 | 46.964 | 78.009 | 1.00 | 84.91 | A | C |
| ATOM | 791 | OD1 | ASP | A | 114 | 72.566 | 46.667 | 78.164 | 1.00 | 84.37 | A | O |
| ATOM | 792 | OD2 | ASP | A | 114 | 70.916 | 47.481 | 76.960 | 1.00 | 86.11 | A | O |
| ATOM | 793 | C | ASP | A | 114 | 67.989 | 46.425 | 79.792 | 1.00 | 80.33 | A | C |
| ATOM | 794 | O | ASP | A | 114 | 67.458 | 45.331 | 79.601 | 1.00 | 78.88 | A | O |
| ATOM | 795 | N | ALA | A | 115 | 67.780 | 47.161 | 80.879 | 1.00 | 78.95 | A | N |
| ATOM | 796 | CA | ALA | A | 115 | 66.891 | 46.725 | 81.946 | 1.00 | 77.50 | A | C |
| ATOM | 797 | CB | ALA | A | 115 | 66.941 | 47.717 | 83.104 | 1.00 | 77.04 | A | C |
| ATOM | 798 | C | ALA | A | 115 | 65.458 | 46.587 | 81.433 | 1.00 | 76.87 | A | C |
| ATOM | 799 | O | ALA | A | 115 | 64.695 | 45.751 | 81.922 | 1.00 | 76.41 | A | O |
| ATOM | 800 | N | LEU | A | 116 | 65.100 | 47.411 | 80.448 | 1.00 | 74.94 | A | N |
| ATOM | 801 | CA | LEU | A | 116 | 63.753 | 47.388 | 79.868 | 1.00 | 72.87 | A | C |
| ATOM | 802 | CB | LEU | A | 116 | 63.433 | 48.726 | 79.178 | 1.00 | 70.09 | A | C |
| ATOM | 803 | CG | LEU | A | 116 | 63.338 | 49.987 | 80.047 | 1.00 | 67.72 | A | C |
| ATOM | 804 | CD1 | LEU | A | 116 | 62.899 | 51.167 | 79.190 | 1.00 | 67.19 | A | C |
| ATOM | 805 | CD2 | LEU | A | 116 | 62.349 | 49.764 | 81.184 | 1.00 | 66.13 | A | C |
| ATOM | 806 | C | LEU | A | 116 | 63.617 | 46.253 | 78.859 | 1.00 | 71.59 | A | C |
| ATOM | 807 | O | LEU | A | 116 | 62.658 | 45.486 | 78.905 | 1.00 | 70.69 | A | O |
| ATOM | 808 | N | VAL | A | 117 | 64.582 | 46.155 | 77.950 | 1.00 | 70.38 | A | N |
| ATOM | 809 | CA | VAL | A | 117 | 64.572 | 45.107 | 76.938 | 1.00 | 69.25 | A | C |
| ATOM | 810 | CB | VAL | A | 117 | 65.832 | 45.174 | 76.035 | 1.00 | 70.05 | A | C |
| ATOM | 811 | CG1 | VAL | A | 117 | 65.869 | 43.970 | 75.100 | 1.00 | 69.44 | A | C |
| ATOM | 812 | CG2 | VAL | A | 117 | 65.827 | 46.462 | 75.225 | 1.00 | 68.98 | A | C |
| ATOM | 813 | C | VAL | A | 117 | 64.513 | 43.735 | 77.596 | 1.00 | 68.29 | A | C |
| ATOM | 814 | O | VAL | A | 117 | 64.000 | 42.786 | 77.008 | 1.00 | 68.46 | A | O |
| ATOM | 815 | N | THR | A | 118 | 65.045 | 43.634 | 78.812 | 1.00 | 66.94 | A | N |
| ATOM | 816 | CA | THR | A | 118 | 65.041 | 42.372 | 79.551 | 1.00 | 65.95 | A | C |
| ATOM | 817 | CB | THR | A | 118 | 66.172 | 42.339 | 80.625 | 1.00 | 65.12 | A | C |
| ATOM | 818 | OG1 | THR | A | 118 | 67.449 | 42.401 | 79.975 | 1.00 | 66.03 | A | O |
| ATOM | 819 | CG2 | THR | A | 118 | 66.098 | 41.058 | 81.452 | 1.00 | 62.95 | A | C |
| ATOM | 820 | C | THR | A | 118 | 63.683 | 42.152 | 80.224 | 1.00 | 64.39 | A | C |
| ATOM | 821 | O | THR | A | 118 | 63.178 | 41.031 | 80.267 | 1.00 | 64.72 | A | O |
| ATOM | 822 | N | ILE | A | 119 | 63.091 | 43.224 | 80.740 | 1.00 | 62.72 | A | N |
| ATOM | 823 | CA | ILE | A | 119 | 61.787 | 43.126 | 81.388 | 1.00 | 62.57 | A | C |
| ATOM | 824 | CB | ILE | A | 119 | 61.378 | 44.456 | 82.039 | 1.00 | 59.21 | A | C |
| ATOM | 825 | CG2 | ILE | A | 119 | 59.941 | 44.373 | 82.532 | 1.00 | 58.41 | A | C |
| ATOM | 826 | CG1 | ILE | A | 119 | 62.317 | 44.779 | 83.194 | 1.00 | 56.25 | A | C |
| ATOM | 827 | CD1 | ILE | A | 119 | 61.955 | 46.057 | 83.906 | 1.00 | 55.40 | A | C |
| ATOM | 828 | C | ILE | A | 119 | 60.720 | 42.750 | 80.359 | 1.00 | 64.44 | A | C |
| ATOM | 829 | O | ILE | A | 119 | 59.844 | 41.918 | 80.616 | 1.00 | 63.80 | A | O |
| ATOM | 830 | N | ARG | A | 120 | 60.806 | 43.367 | 79.186 | 1.00 | 66.12 | A | N |
| ATOM | 831 | CA | ARG | A | 120 | 59.855 | 43.106 | 78.112 | 1.00 | 67.75 | A | C |
| ATOM | 832 | CB | ARG | A | 120 | 60.217 | 43.938 | 76.882 | 1.00 | 68.59 | A | C |
| ATOM | 833 | CG | ARG | A | 120 | 59.345 | 43.671 | 75.672 | 1.00 | 70.06 | A | C |
| ATOM | 834 | CD | ARG | A | 120 | 59.747 | 44.560 | 74.511 | 1.00 | 72.45 | A | C |
| ATOM | 835 | NE | ARG | A | 120 | 61.136 | 44.355 | 74.106 | 1.00 | 77.09 | A | N |
| ATOM | 836 | CZ | ARG | A | 120 | 61.807 | 45.172 | 73.297 | 1.00 | 80.07 | A | C |
| ATOM | 837 | NH1 | ARG | A | 120 | 61.217 | 46.256 | 72.808 | 1.00 | 79.95 | A | N |
| ATOM | 838 | NH2 | ARG | A | 120 | 63.066 | 44.905 | 72.970 | 1.00 | 80.35 | A | N |
| ATOM | 839 | C | ARG | A | 120 | 59.822 | 41.631 | 77.737 | 1.00 | 67.58 | A | C |
| ATOM | 840 | O | ARG | A | 120 | 58.751 | 41.033 | 77.622 | 1.00 | 67.09 | A | O |
| ATOM | 841 | N | ASN | A | 121 | 61.001 | 41.050 | 77.547 | 1.00 | 67.97 | A | N |
| ATOM | 842 | CA | ASN | A | 121 | 61.096 | 39.649 | 77.172 | 1.00 | 67.97 | A | C |
| ATOM | 843 | CB | ASN | A | 121 | 62.514 | 39.317 | 76.717 | 1.00 | 71.57 | A | C |
| ATOM | 844 | CG | ASN | A | 121 | 62.972 | 40.194 | 75.565 | 1.00 | 74.09 | A | C |
| ATOM | 845 | OD1 | ASN | A | 121 | 62.178 | 40.566 | 74.698 | 1.00 | 73.69 | A | O |
| ATOM | 846 | ND2 | ASN | A | 121 | 64.259 | 40.521 | 75.544 | 1.00 | 76.02 | A | N |
| ATOM | 847 | C | ASN | A | 121 | 60.678 | 38.730 | 78.305 | 1.00 | 65.90 | A | C |
| ATOM | 848 | O | ASN | A | 121 | 60.164 | 37.645 | 78.069 | 1.00 | 65.64 | A | O |
| ATOM | 849 | N | ARG | A | 122 | 60.892 | 39.167 | 79.537 | 1.00 | 64.68 | A | N |
| ATOM | 850 | CA | ARG | A | 122 | 60.504 | 38.367 | 80.693 | 1.00 | 62.79 | A | C |
| ATOM | 851 | CB | ARG | A | 122 | 60.956 | 39.074 | 81.978 | 1.00 | 63.72 | A | C |
| ATOM | 852 | CG | ARG | A | 122 | 60.526 | 38.426 | 83.295 | 1.00 | 64.18 | A | C |
| ATOM | 853 | CD | ARG | A | 122 | 61.018 | 39.271 | 84.470 | 1.00 | 66.34 | A | C |
| ATOM | 854 | NE | ARG | A | 122 | 62.469 | 39.416 | 84.425 | 1.00 | 68.16 | A | N |
| ATOM | 855 | CZ | ARG | A | 122 | 63.136 | 40.490 | 84.830 | 1.00 | 71.52 | A | C |
| ATOM | 856 | NH1 | ARG | A | 122 | 62.487 | 41.538 | 85.322 | 1.00 | 71.80 | A | N |
| ATOM | 857 | NH2 | ARG | A | 122 | 64.458 | 40.521 | 84.726 | 1.00 | 72.25 | A | N |
| ATOM | 858 | C | ARG | A | 122 | 58.982 | 38.184 | 80.687 | 1.00 | 60.78 | A | C |
| ATOM | 859 | O | ARG | A | 122 | 58.458 | 37.193 | 81.206 | 1.00 | 60.26 | A | O |
| ATOM | 860 | N | HIS | A | 123 | 58.282 | 39.138 | 80.076 | 1.00 | 57.04 | A | N |
| ATOM | 861 | CA | HIS | A | 123 | 56.822 | 39.111 | 80.021 | 1.00 | 53.93 | A | C |
| ATOM | 862 | CB | HIS | A | 123 | 56.275 | 40.483 | 80.421 | 1.00 | 50.94 | A | C |
| ATOM | 863 | CG | HIS | A | 123 | 56.592 | 40.874 | 81.828 | 1.00 | 49.08 | A | C |
| ATOM | 864 | CD2 | HIS | A | 123 | 57.749 | 41.285 | 82.398 | 1.00 | 47.29 | A | C |
| ATOM | 865 | ND1 | HIS | A | 123 | 55.654 | 40.840 | 82.840 | 1.00 | 48.94 | A | N |
| ATOM | 866 | CE1 | HIS | A | 123 | 56.223 | 41.214 | 83.974 | 1.00 | 49.20 | A | C |
| ATOM | 867 | NE2 | HIS | A | 123 | 57.493 | 41.490 | 83.732 | 1.00 | 50.33 | A | N |
| ATOM | 868 | C | HIS | A | 123 | 56.220 | 38.717 | 78.668 | 1.00 | 52.72 | A | C |
| ATOM | 869 | O | HIS | A | 123 | 55.010 | 38.783 | 78.487 | 1.00 | 53.00 | A | O |
| ATOM | 870 | N | ASN | A | 124 | 57.042 | 38.291 | 77.722 | 1.00 | 53.05 | A | N |
| ATOM | 871 | CA | ASN | A | 124 | 56.521 | 37.928 | 76.407 | 1.00 | 53.54 | A | C |
| ATOM | 872 | CB | ASN | A | 124 | 57.655 | 37.467 | 75.494 | 1.00 | 53.26 | A | C |
| ATOM | 873 | CG | ASN | A | 124 | 58.205 | 38.591 | 74.656 | 1.00 | 59.21 | A | C |
| ATOM | 874 | OD1 | ASN | A | 124 | 57.449 | 39.321 | 74.015 | 1.00 | 61.37 | A | O |
| ATOM | 875 | ND2 | ASN | A | 124 | 59.525 | 38.738 | 74.646 | 1.00 | 62.95 | A | N |
| ATOM | 876 | C | ASN | A | 124 | 55.403 | 36.898 | 76.351 | 1.00 | 52.85 | A | C |
| ATOM | 877 | O | ASN | A | 124 | 54.549 | 36.960 | 75.471 | 1.00 | 54.61 | A | O |
| ATOM | 878 | N | ASP | A | 125 | 55.386 | 35.955 | 77.281 | 1.00 | 53.53 | A | N |
| ATOM | 879 | CA | ASP | A | 125 | 54.366 | 34.913 | 77.256 | 1.00 | 53.22 | A | C |
| ATOM | 880 | CB | ASP | A | 125 | 55.039 | 33.541 | 77.411 | 1.00 | 56.53 | A | C |
| ATOM | 881 | CG | ASP | A | 125 | 55.953 | 33.203 | 76.234 | 1.00 | 61.28 | A | C |
| ATOM | 882 | OD1 | ASP | A | 125 | 55.438 | 33.052 | 75.101 | 1.00 | 61.87 | A | O |
| ATOM | 883 | OD2 | ASP | A | 125 | 57.183 | 33.097 | 76.433 | 1.00 | 62.33 | A | O |
| ATOM | 884 | C | ASP | A | 125 | 53.263 | 35.077 | 78.293 | 1.00 | 51.87 | A | C |
| ATOM | 885 | O | ASP | A | 125 | 52.608 | 34.103 | 78.673 | 1.00 | 53.55 | A | O |
| ATOM | 886 | N | VAL | A | 126 | 53.040 | 36.310 | 78.733 | 1.00 | 48.65 | A | N |
| ATOM | 887 | CA | VAL | A | 126 | 52.011 | 36.576 | 79.730 | 1.00 | 48.33 | A | C |
| ATOM | 888 | CB | VAL | A | 126 | 51.991 | 38.076 | 80.141 | 1.00 | 49.15 | A | C |
| ATOM | 889 | CG1 | VAL | A | 126 | 50.820 | 38.340 | 81.092 | 1.00 | 47.99 | A | C |
| ATOM | 890 | CG2 | VAL | A | 126 | 53.300 | 38.455 | 80.814 | 1.00 | 48.96 | A | C |
| ATOM | 891 | C | VAL | A | 126 | 50.609 | 36.186 | 79.262 | 1.00 | 48.57 | A | C |
| ATOM | 892 | O | VAL | A | 126 | 49.805 | 35.688 | 80.063 | 1.00 | 48.54 | A | O |
| ATOM | 893 | N | VAL | A | 127 | 50.299 | 36.415 | 77.984 | 1.00 | 47.55 | A | N |
| ATOM | 894 | CA | VAL | A | 127 | 48.969 | 36.075 | 77.488 | 1.00 | 46.60 | A | C |
| ATOM | 895 | CB | VAL | A | 127 | 48.736 | 36.553 | 76.026 | 1.00 | 49.20 | A | C |
| ATOM | 896 | CG1 | VAL | A | 127 | 47.292 | 36.227 | 75.605 | 1.00 | 45.02 | A | C |
| ATOM | 897 | CG2 | VAL | A | 127 | 48.993 | 38.058 | 75.903 | 1.00 | 50.51 | A | C |
| ATOM | 898 | C | VAL | A | 127 | 48.681 | 34.577 | 77.568 | 1.00 | 46.02 | A | C |
| ATOM | 899 | O | VAL | A | 127 | 47.728 | 34.169 | 78.230 | 1.00 | 46.37 | A | O |
| ATOM | 900 | N | PRO | A | 128 | 49.489 | 33.737 | 76.896 | 1.00 | 46.41 | A | N |
| ATOM | 901 | CD | PRO | A | 128 | 50.698 | 34.033 | 76.102 | 1.00 | 43.95 | A | C |
| ATOM | 902 | CA | PRO | A | 128 | 49.233 | 32.288 | 76.959 | 1.00 | 45.69 | A | C |
| ATOM | 903 | CB | PRO | A | 128 | 50.408 | 31.690 | 76.185 | 1.00 | 43.74 | A | C |
| ATOM | 904 | CG | PRO | A | 128 | 50.825 | 32.803 | 75.248 | 1.00 | 46.46 | A | C |
| ATOM | 905 | C | PRO | A | 128 | 49.203 | 31.784 | 78.415 | 1.00 | 47.10 | A | C |
| ATOM | 906 | O | PRO | A | 128 | 48.340 | 30.979 | 78.800 | 1.00 | 46.04 | A | O |
| ATOM | 907 | N | THR | A | 129 | 50.152 | 32.267 | 79.220 | 1.00 | 47.77 | A | N |
| ATOM | 908 | CA | THR | A | 129 | 50.252 | 31.855 | 80.619 | 1.00 | 48.14 | A | C |
| ATOM | 909 | CB | THR | A | 129 | 51.423 | 32.534 | 81.328 | 1.00 | 48.42 | A | C |
| ATOM | 910 | OG1 | THR | A | 129 | 52.659 | 32.130 | 80.722 | 1.00 | 50.09 | A | O |
| ATOM | 911 | CG2 | THR | A | 129 | 51.426 | 32.159 | 82.806 | 1.00 | 47.05 | A | C |
| ATOM | 912 | C | THR | A | 129 | 48.986 | 32.169 | 81.393 | 1.00 | 49.86 | A | C |
| ATOM | 913 | O | THR | A | 129 | 48.496 | 31.339 | 82.162 | 1.00 | 49.48 | A | O |
| ATOM | 914 | N | MET | A | 130 | 48.459 | 33.372 | 81.200 | 1.00 | 49.90 | A | N |
| ATOM | 915 | CA | MET | A | 130 | 47.231 | 33.759 | 81.883 | 1.00 | 50.91 | A | C |
| ATOM | 916 | CB | MET | A | 130 | 46.978 | 35.256 | 81.696 | 1.00 | 51.92 | A | C |
| ATOM | 917 | CG | MET | A | 130 | 47.931 | 36.159 | 82.471 | 1.00 | 53.13 | A | C |
| ATOM | 918 | SD | MET | A | 130 | 47.790 | 35.984 | 84.279 | 1.00 | 54.48 | A | S |
| ATOM | 919 | CE | MET | A | 130 | 46.135 | 36.467 | 84.581 | 1.00 | 48.51 | A | C |
| ATOM | 920 | C | MET | A | 130 | 46.046 | 32.949 | 81.337 | 1.00 | 51.36 | A | C |
| ATOM | 921 | O | MET | A | 130 | 45.137 | 32.575 | 82.088 | 1.00 | 49.05 | A | O |
| ATOM | 922 | N | ALA | A | 131 | 46.057 | 32.692 | 80.028 | 1.00 | 50.89 | A | N |
| ATOM | 923 | CA | ALA | A | 131 | 44.999 | 31.911 | 79.382 | 1.00 | 51.65 | A | C |
| ATOM | 924 | CB | ALA | A | 131 | 45.280 | 31.792 | 77.878 | 1.00 | 49.47 | A | C |
| ATOM | 925 | C | ALA | A | 131 | 44.930 | 30.512 | 80.016 | 1.00 | 51.17 | A | C |
| ATOM | 926 | O | ALA | A | 131 | 43.858 | 29.915 | 80.149 | 1.00 | 48.30 | A | O |
| ATOM | 927 | N | GLN | A | 132 | 46.092 | 30.000 | 80.403 | 1.00 | 53.29 | A | N |
| ATOM | 928 | CA | GLN | A | 132 | 46.188 | 28.686 | 81.033 | 1.00 | 54.90 | A | C |
| ATOM | 929 | CB | GLN | A | 132 | 47.651 | 28.345 | 81.269 | 1.00 | 59.70 | A | C |
| ATOM | 930 | CG | GLN | A | 132 | 47.895 | 26.953 | 81.772 | 1.00 | 69.20 | A | C |
| ATOM | 931 | CD | GLN | A | 132 | 49.317 | 26.536 | 81.520 | 1.00 | 76.60 | A | C |
| ATOM | 932 | OE1 | GLN | A | 132 | 49.725 | 26.356 | 80.373 | 1.00 | 82.72 | A | O |
| ATOM | 933 | NE2 | GLN | A | 132 | 50.093 | 26.399 | 82.583 | 1.00 | 79.85 | A | N |
| ATOM | 934 | C | GLN | A | 132 | 45.433 | 28.684 | 82.359 | 1.00 | 52.97 | A | C |
| ATOM | 935 | O | GLN | A | 132 | 44.787 | 27.694 | 82.719 | 1.00 | 50.01 | A | O |
| ATOM | 936 | N | GLY | A | 133 | 45.520 | 29.804 | 83.075 | 1.00 | 50.73 | A | N |
| ATOM | 937 | CA | GLY | A | 133 | 44.825 | 29.934 | 84.339 | 1.00 | 50.47 | A | C |
| ATOM | 938 | C | GLY | A | 133 | 43.329 | 29.985 | 84.116 | 1.00 | 52.72 | A | C |
| ATOM | 939 | O | GLY | A | 133 | 42.551 | 29.574 | 84.981 | 1.00 | 52.74 | A | O |
| ATOM | 940 | N | VAL | A | 134 | 42.914 | 30.501 | 82.962 | 1.00 | 55.57 | A | N |
| ATOM | 941 | CA | VAL | A | 134 | 41.492 | 30.585 | 82.651 | 1.00 | 57.75 | A | C |
| ATOM | 942 | CB | VAL | A | 134 | 41.238 | 31.372 | 81.347 | 1.00 | 57.21 | A | C |
| ATOM | 943 | CG1 | VAL | A | 134 | 39.754 | 31.324 | 80.983 | 1.00 | 57.86 | A | C |
| ATOM | 944 | CG2 | VAL | A | 134 | 41.673 | 32.815 | 81.525 | 1.00 | 58.18 | A | C |
| ATOM | 945 | C | VAL | A | 134 | 40.958 | 29.170 | 82.492 | 1.00 | 58.47 | A | C |
| ATOM | 946 | O | VAL | A | 134 | 39.905 | 28.821 | 83.037 | 1.00 | 57.13 | A | O |
| ATOM | 947 | N | LEU | A | 135 | 41.695 | 28.355 | 81.747 | 1.00 | 59.09 | A | N |
| ATOM | 948 | CA | LEU | A | 135 | 41.301 | 26.971 | 81.534 | 1.00 | 61.51 | A | C |
| ATOM | 949 | CB | LEU | A | 135 | 42.235 | 26.300 | 80.527 | 1.00 | 62.15 | A | C |
| ATOM | 950 | CG | LEU | A | 135 | 41.777 | 24.903 | 80.103 | 1.00 | 66.24 | A | C |
| ATOM | 951 | CD1 | LEU | A | 135 | 40.344 | 24.967 | 79.557 | 1.00 | 65.40 | A | C |
| ATOM | 952 | CD2 | LEU | A | 135 | 42.736 | 24.348 | 79.060 | 1.00 | 65.39 | A | C |
| ATOM | 953 | C | LEU | A | 135 | 41.323 | 26.208 | 82.862 | 1.00 | 61.01 | A | C |
| ATOM | 954 | O | LEU | A | 135 | 40.460 | 25.365 | 83.115 | 1.00 | 61.85 | A | O |
| ATOM | 955 | N | GLU | A | 136 | 42.309 | 26.506 | 83.704 | 1.00 | 59.51 | A | N |
| ATOM | 956 | CA | GLU | A | 136 | 42.407 | 25.861 | 85.004 | 1.00 | 59.39 | A | C |
| ATOM | 957 | CB | GLU | A | 136 | 43.619 | 26.384 | 85.781 | 1.00 | 59.74 | A | C |
| ATOM | 958 | CG | GLU | A | 136 | 44.805 | 25.439 | 85.767 | 1.00 | 61.77 | A | C |
| ATOM | 959 | CD | GLU | A | 136 | 46.102 | 26.117 | 86.139 | 1.00 | 62.71 | A | C |
| ATOM | 960 | OE1 | GLU | A | 136 | 46.121 | 26.875 | 87.137 | 1.00 | 64.40 | A | O |
| ATOM | 961 | OE2 | GLU | A | 136 | 47.104 | 25.880 | 85.432 | 1.00 | 62.39 | A | O |
| ATOM | 962 | C | GLU | A | 136 | 41.137 | 26.175 | 85.769 | 1.00 | 59.21 | A | C |
| ATOM | 963 | O | GLU | A | 136 | 40.508 | 25.283 | 86.348 | 1.00 | 60.50 | A | O |
| ATOM | 964 | N | TYR | A | 137 | 40.749 | 27.446 | 85.752 | 1.00 | 57.53 | A | N |
| ATOM | 965 | CA | TYR | A | 137 | 39.555 | 27.877 | 86.457 | 1.00 | 56.80 | A | C |
| ATOM | 966 | CB | TYR | A | 137 | 39.377 | 29.390 | 86.309 | 1.00 | 58.08 | A | C |
| ATOM | 967 | CG | TYR | A | 137 | 38.263 | 29.935 | 87.160 | 1.00 | 57.32 | A | C |
| ATOM | 968 | CD1 | TYR | A | 137 | 36.931 | 29.700 | 86.825 | 1.00 | 56.81 | A | C |
| ATOM | 969 | CE1 | TYR | A | 137 | 35.899 | 30.116 | 87.655 | 1.00 | 59.15 | A | C |
| ATOM | 970 | CD2 | TYR | A | 137 | 38.540 | 30.613 | 88.348 | 1.00 | 57.28 | A | C |
| ATOM | 971 | CE2 | TYR | A | 137 | 37.513 | 31.034 | 89.187 | 1.00 | 59.30 | A | C |
| ATOM | 972 | CZ | TYR | A | 137 | 36.195 | 30.780 | 88.833 | 1.00 | 58.82 | A | C |
| ATOM | 973 | OH | TYR | A | 137 | 35.170 | 31.174 | 89.656 | 1.00 | 60.08 | A | O |
| ATOM | 974 | C | TYR | A | 137 | 38.313 | 27.149 | 85.950 | 1.00 | 57.96 | A | C |
| ATOM | 975 | O | TYR | A | 137 | 37.544 | 26.608 | 86.740 | 1.00 | 56.94 | A | O |
| ATOM | 976 | N | LYS | A | 138 | 38.118 | 27.141 | 84.635 | 1.00 | 60.28 | A | N |
| ATOM | 977 | CA | LYS | A | 138 | 36.968 | 26.477 | 84.017 | 1.00 | 63.12 | A | C |
| ATOM | 978 | CB | LYS | A | 138 | 37.133 | 26.454 | 82.487 | 1.00 | 64.27 | A | C |
| ATOM | 979 | CG | LYS | A | 138 | 36.936 | 27.810 | 81.801 | 1.00 | 69.33 | A | C |
| ATOM | 980 | CD | LYS | A | 138 | 37.319 | 27.774 | 80.319 | 1.00 | 71.28 | A | C |
| ATOM | 981 | CE | LYS | A | 138 | 36.499 | 26.762 | 79.527 | 1.00 | 72.57 | A | C |
| ATOM | 982 | NZ | LYS | A | 138 | 35.042 | 27.059 | 79.559 | 1.00 | 74.24 | A | N |
| ATOM | 983 | C | LYS | A | 138 | 36.763 | 25.047 | 84.529 | 1.00 | 64.11 | A | C |
| ATOM | 984 | O | LYS | A | 138 | 35.655 | 24.669 | 84.918 | 1.00 | 62.65 | A | O |
| ATOM | 985 | N | ASP | A | 139 | 37.836 | 24.259 | 84.532 | 1.00 | 64.72 | A | N |
| ATOM | 986 | CA | ASP | A | 139 | 37.765 | 22.872 | 84.981 | 1.00 | 65.08 | A | C |
| ATOM | 987 | CB | ASP | A | 139 | 38.913 | 22.059 | 84.369 | 1.00 | 63.99 | A | C |
| ATOM | 988 | CG | ASP | A | 139 | 38.914 | 22.098 | 82.856 | 1.00 | 64.65 | A | C |
| ATOM | 989 | OD1 | ASP | A | 139 | 37.823 | 22.006 | 82.258 | 1.00 | 66.05 | A | O |
| ATOM | 990 | OD2 | ASP | A | 139 | 40.008 | 22.207 | 82.262 | 1.00 | 64.73 | A | O |
| ATOM | 991 | C | ASP | A | 139 | 37.784 | 22.705 | 86.505 | 1.00 | 64.51 | A | C |
| ATOM | 992 | O | ASP | A | 139 | 37.302 | 21.702 | 87.036 | 1.00 | 64.50 | A | O |
| ATOM | 993 | N | THR | A | 140 | 38.333 | 23.686 | 87.206 | 1.00 | 63.73 | A | N |
| ATOM | 994 | CA | THR | A | 140 | 38.410 | 23.615 | 88.657 | 1.00 | 64.79 | A | C |
| ATOM | 995 | CB | THR | A | 140 | 39.638 | 24.387 | 89.175 | 1.00 | 64.43 | A | C |
| ATOM | 996 | OG1 | THR | A | 140 | 40.833 | 23.779 | 88.671 | 1.00 | 63.78 | A | O |
| ATOM | 997 | CG2 | THR | A | 140 | 39.668 | 24.378 | 90.700 | 1.00 | 64.16 | A | C |
| ATOM | 998 | C | THR | A | 140 | 37.170 | 24.136 | 89.393 | 1.00 | 66.24 | A | C |
| ATOM | 999 | O | THR | A | 140 | 36.801 | 23.595 | 90.439 | 1.00 | 63.56 | A | O |
| ATOM | 1000 | N | TYR | A | 141 | 36.534 | 25.175 | 88.847 | 1.00 | 68.97 | A | N |
| ATOM | 1001 | CA | TYR | A | 141 | 35.360 | 25.778 | 89.479 | 1.00 | 71.51 | A | C |
| ATOM | 1002 | CB | TYR | A | 141 | 35.743 | 27.135 | 90.079 | 1.00 | 72.04 | A | C |
| ATOM | 1003 | CG | TYR | A | 141 | 36.731 | 27.020 | 91.216 | 1.00 | 74.21 | A | C |
| ATOM | 1004 | CD1 | TYR | A | 141 | 38.060 | 27.422 | 91.061 | 1.00 | 74.55 | A | C |
| ATOM | 1005 | CE1 | TYR | A | 141 | 38.982 | 27.267 | 92.095 | 1.00 | 76.70 | A | C |
| ATOM | 1006 | CD2 | TYR | A | 141 | 36.347 | 26.462 | 92.437 | 1.00 | 75.03 | A | C |
| ATOM | 1007 | CE2 | TYR | A | 141 | 37.258 | 26.301 | 93.475 | 1.00 | 76.73 | A | C |
| ATOM | 1008 | CZ | TYR | A | 141 | 38.573 | 26.703 | 93.300 | 1.00 | 77.99 | A | C |
| ATOM | 1009 | OH | TYR | A | 141 | 39.478 | 26.525 | 94.325 | 1.00 | 80.44 | A | O |
| ATOM | 1010 | C | TYR | A | 141 | 34.133 | 25.943 | 88.582 | 1.00 | 72.94 | A | C |
| ATOM | 1011 | O | TYR | A | 141 | 33.050 | 26.283 | 89.063 | 1.00 | 72.00 | A | O |
| ATOM | 1012 | N | GLY | A | 142 | 34.295 | 25.708 | 87.284 | 1.00 | 75.47 | A | N |
| ATOM | 1013 | CA | GLY | A | 142 | 33.165 | 25.829 | 86.377 | 1.00 | 78.56 | A | C |
| ATOM | 1014 | C | GLY | A | 142 | 33.080 | 27.150 | 85.641 | 1.00 | 80.26 | A | C |
| ATOM | 1015 | O | GLY | A | 142 | 33.733 | 28.119 | 86.022 | 1.00 | 79.99 | A | O |
| ATOM | 1016 | N | ASP | A | 143 | 32.261 | 27.190 | 84.593 | 1.00 | 83.18 | A | N |
| ATOM | 1017 | CA | ASP | A | 143 | 32.102 | 28.397 | 83.789 | 1.00 | 87.05 | A | C |
| ATOM | 1018 | CB | ASP | A | 143 | 32.461 | 28.090 | 82.327 | 1.00 | 89.64 | A | C |
| ATOM | 1019 | CG | ASP | A | 143 | 31.575 | 27.017 | 81.713 | 1.00 | 92.88 | A | C |
| ATOM | 1020 | OD1 | ASP | A | 143 | 30.981 | 26.220 | 82.472 | 1.00 | 93.77 | A | O |
| ATOM | 1021 | OD2 | ASP | A | 143 | 31.486 | 26.962 | 80.466 | 1.00 | 94.08 | A | O |
| ATOM | 1022 | C | ASP | A | 143 | 30.712 | 29.036 | 83.870 | 1.00 | 88.17 | A | C |
| ATOM | 1023 | O | ASP | A | 143 | 29.873 | 28.850 | 82.991 | 1.00 | 89.46 | A | O |
| ATOM | 1024 | N | ASP | A | 144 | 30.485 | 29.803 | 84.932 | 1.00 | 89.77 | A | N |
| ATOM | 1025 | CA | ASP | A | 144 | 29.212 | 30.491 | 85.150 | 1.00 | 91.39 | A | C |
| ATOM | 1026 | CB | ASP | A | 144 | 28.743 | 30.265 | 86.588 | 1.00 | 91.27 | A | C |
| ATOM | 1027 | CG | ASP | A | 144 | 29.860 | 30.465 | 87.599 | 1.00 | 91.33 | A | C |
| ATOM | 1028 | OD1 | ASP | A | 144 | 30.795 | 29.638 | 87.611 | 1.00 | 92.36 | A | O |
| ATOM | 1029 | OD2 | ASP | A | 144 | 29.812 | 31.448 | 88.372 | 1.00 | 90.89 | A | O |
| ATOM | 1030 | C | ASP | A | 144 | 29.353 | 31.995 | 84.888 | 1.00 | 91.77 | A | C |
| ATOM | 1031 | O | ASP | A | 144 | 30.459 | 32.540 | 84.925 | 1.00 | 92.16 | A | O |
| ATOM | 1032 | N | PRO | A | 145 | 28.229 | 32.688 | 84.631 | 1.00 | 91.18 | A | N |
| ATOM | 1033 | CD | PRO | A | 145 | 26.862 | 32.146 | 84.505 | 1.00 | 90.78 | A | C |
| ATOM | 1034 | CA | PRO | A | 145 | 28.233 | 34.130 | 84.363 | 1.00 | 90.06 | A | C |
| ATOM | 1035 | CB | PRO | A | 145 | 26.752 | 34.482 | 84.412 | 1.00 | 90.16 | A | C |
| ATOM | 1036 | CG | PRO | A | 145 | 26.127 | 33.276 | 83.807 | 1.00 | 90.14 | A | C |
| ATOM | 1037 | C | PRO | A | 145 | 29.074 | 34.996 | 85.307 | 1.00 | 89.20 | A | C |
| ATOM | 1038 | O | PRO | A | 145 | 29.829 | 35.854 | 84.851 | 1.00 | 88.90 | A | O |
| ATOM | 1039 | N | VAL | A | 146 | 28.946 | 34.785 | 86.614 | 1.00 | 87.71 | A | N |
| ATOM | 1040 | CA | VAL | A | 146 | 29.713 | 35.583 | 87.563 | 1.00 | 86.87 | A | C |
| ATOM | 1041 | CB | VAL | A | 146 | 29.276 | 35.312 | 89.015 | 1.00 | 86.35 | A | C |
| ATOM | 1042 | CG1 | VAL | A | 146 | 30.070 | 36.195 | 89.973 | 1.00 | 86.20 | A | C |
| ATOM | 1043 | CG2 | VAL | A | 146 | 27.793 | 35.587 | 89.160 | 1.00 | 86.25 | A | C |
| ATOM | 1044 | C | VAL | A | 146 | 31.215 | 35.337 | 87.427 | 1.00 | 86.61 | A | C |
| ATOM | 1045 | O | VAL | A | 146 | 32.012 | 36.268 | 87.564 | 1.00 | 86.93 | A | O |
| ATOM | 1046 | N | SER | A | 147 | 31.603 | 34.092 | 87.157 | 1.00 | 85.90 | A | N |
| ATOM | 1047 | CA | SER | A | 147 | 33.020 | 33.766 | 86.985 | 1.00 | 85.84 | A | C |
| ATOM | 1048 | CB | SER | A | 147 | 33.223 | 32.251 | 86.837 | 1.00 | 85.75 | A | C |
| ATOM | 1049 | OG | SER | A | 147 | 32.895 | 31.562 | 88.031 | 1.00 | 85.29 | A | O |
| ATOM | 1050 | C | SER | A | 147 | 33.503 | 34.468 | 85.723 | 1.00 | 85.75 | A | C |
| ATOM | 1051 | O | SER | A | 147 | 34.560 | 35.105 | 85.706 | 1.00 | 85.74 | A | O |
| ATOM | 1052 | N | ASN | A | 148 | 32.706 | 34.340 | 84.668 | 1.00 | 84.81 | A | N |
| ATOM | 1053 | CA | ASN | A | 148 | 32.995 | 34.957 | 83.380 | 1.00 | 84.61 | A | C |
| ATOM | 1054 | CB | ASN | A | 148 | 31.854 | 34.655 | 82.411 | 1.00 | 83.75 | A | C |
| ATOM | 1055 | CG | ASN | A | 148 | 31.994 | 35.393 | 81.102 | 1.00 | 84.62 | A | C |
| ATOM | 1056 | OD1 | ASN | A | 148 | 32.851 | 35.067 | 80.283 | 1.00 | 84.01 | A | O |
| ATOM | 1057 | ND2 | ASN | A | 148 | 31.154 | 36.404 | 80.899 | 1.00 | 84.52 | A | N |
| ATOM | 1058 | C | ASN | A | 148 | 33.142 | 36.473 | 83.541 | 1.00 | 84.47 | A | C |
| ATOM | 1059 | O | ASN | A | 148 | 34.085 | 37.082 | 83.036 | 1.00 | 83.68 | A | O |
| ATOM | 1060 | N | GLN | A | 149 | 32.193 | 37.064 | 84.258 | 1.00 | 84.48 | A | N |
| ATOM | 1061 | CA | GLN | A | 149 | 32.158 | 38.496 | 84.511 | 1.00 | 84.65 | A | C |
| ATOM | 1062 | CB | GLN | A | 149 | 30.930 | 38.813 | 85.360 | 1.00 | 89.50 | A | C |
| ATOM | 1063 | CG | GLN | A | 149 | 30.552 | 40.268 | 85.440 | 1.00 | 96.51 | A | C |
| ATOM | 1064 | CD | GLN | A | 149 | 29.214 | 40.454 | 86.130 | 1.00 | 101.15 | A | C |
| ATOM | 1065 | OE1 | GLN | A | 149 | 28.192 | 39.928 | 85.680 | 1.00 | 102.38 | A | O |
| ATOM | 1066 | NE2 | GLN | A | 149 | 29.213 | 41.197 | 87.234 | 1.00 | 103.87 | A | N |
| ATOM | 1067 | C | GLN | A | 149 | 33.422 | 38.994 | 85.210 | 1.00 | 82.00 | A | C |
| ATOM | 1068 | O | GLN | A | 149 | 34.060 | 39.937 | 84.743 | 1.00 | 81.81 | A | O |
| ATOM | 1069 | N | ASN | A | 150 | 33.786 | 38.360 | 86.321 | 1.00 | 78.61 | A | N |
| ATOM | 1070 | CA | ASN | A | 150 | 34.965 | 38.770 | 87.071 | 1.00 | 75.25 | A | C |
| ATOM | 1071 | CB | ASN | A | 150 | 35.061 | 37.987 | 88.376 | 1.00 | 78.17 | A | C |
| ATOM | 1072 | CG | ASN | A | 150 | 33.778 | 38.040 | 89.170 | 1.00 | 80.25 | A | C |
| ATOM | 1073 | OD1 | ASN | A | 150 | 33.123 | 39.084 | 89.243 | 1.00 | 79.83 | A | O |
| ATOM | 1074 | ND2 | ASN | A | 150 | 33.409 | 36.916 | 89.776 | 1.00 | 81.60 | A | N |
| ATOM | 1075 | C | ASN | A | 150 | 36.237 | 38.592 | 86.262 | 1.00 | 71.45 | A | C |
| ATOM | 1076 | O | ASN | A | 150 | 37.103 | 39.470 | 86.260 | 1.00 | 70.30 | A | O |
| ATOM | 1077 | N | ILE | A | 151 | 36.351 | 37.458 | 85.576 | 1.00 | 67.56 | A | N |
| ATOM | 1078 | CA | ILE | A | 151 | 37.526 | 37.190 | 84.751 | 1.00 | 64.24 | A | C |
| ATOM | 1079 | CB | ILE | A | 151 | 37.465 | 35.775 | 84.117 | 1.00 | 63.97 | A | C |
| ATOM | 1080 | CG2 | ILE | A | 151 | 38.592 | 35.606 | 83.093 | 1.00 | 62.40 | A | C |
| ATOM | 1081 | CG1 | ILE | A | 151 | 37.564 | 34.710 | 85.216 | 1.00 | 65.13 | A | C |
| ATOM | 1082 | CD1 | ILE | A | 151 | 37.411 | 33.275 | 84.720 | 1.00 | 65.64 | A | C |
| ATOM | 1083 | C | ILE | A | 151 | 37.605 | 38.233 | 83.636 | 1.00 | 61.82 | A | C |
| ATOM | 1084 | O | ILE | A | 151 | 38.665 | 38.813 | 83.379 | 1.00 | 60.64 | A | O |
| ATOM | 1085 | N | GLN | A | 152 | 36.466 | 38.459 | 82.986 | 1.00 | 58.06 | A | N |
| ATOM | 1086 | CA | GLN | A | 152 | 36.359 | 39.420 | 81.897 | 1.00 | 57.80 | A | C |
| ATOM | 1087 | CB | GLN | A | 152 | 34.903 | 39.494 | 81.423 | 1.00 | 57.35 | A | C |
| ATOM | 1088 | CG | GLN | A | 152 | 34.700 | 40.101 | 80.044 | 1.00 | 59.56 | A | C |
| ATOM | 1089 | CD | GLN | A | 152 | 35.440 | 39.344 | 78.942 | 1.00 | 62.30 | A | C |
| ATOM | 1090 | OE1 | GLN | A | 152 | 35.525 | 38.112 | 78.957 | 1.00 | 58.90 | A | O |
| ATOM | 1091 | NE2 | GLN | A | 152 | 35.968 | 40.085 | 77.973 | 1.00 | 62.37 | A | N |
| ATOM | 1092 | C | GLN | A | 152 | 36.818 | 40.784 | 82.416 | 1.00 | 55.43 | A | C |
| ATOM | 1093 | O | GLN | A | 152 | 37.710 | 41.418 | 81.847 | 1.00 | 52.28 | A | O |
| ATOM | 1094 | N | TYR | A | 153 | 36.210 | 41.208 | 83.518 | 1.00 | 55.54 | A | N |
| ATOM | 1095 | CA | TYR | A | 153 | 36.542 | 42.480 | 84.138 | 1.00 | 56.89 | A | C |
| ATOM | 1096 | CB | TYR | A | 153 | 35.776 | 42.644 | 85.449 | 1.00 | 60.13 | A | C |
| ATOM | 1097 | CG | TYR | A | 153 | 36.038 | 43.971 | 86.106 | 1.00 | 66.55 | A | C |
| ATOM | 1098 | CD1 | TYR | A | 153 | 35.470 | 45.143 | 85.601 | 1.00 | 69.22 | A | C |
| ATOM | 1099 | CE1 | TYR | A | 153 | 35.769 | 46.383 | 86.158 | 1.00 | 71.31 | A | C |
| ATOM | 1100 | CD2 | TYR | A | 153 | 36.907 | 44.070 | 87.190 | 1.00 | 68.51 | A | C |
| ATOM | 1101 | CE2 | TYR | A | 153 | 37.209 | 45.300 | 87.754 | 1.00 | 72.16 | A | C |
| ATOM | 1102 | CZ | TYR | A | 153 | 36.639 | 46.452 | 87.232 | 1.00 | 72.60 | A | C |
| ATOM | 1103 | OH | TYR | A | 153 | 36.956 | 47.673 | 87.775 | 1.00 | 75.63 | A | O |
| ATOM | 1104 | C | TYR | A | 153 | 38.038 | 42.596 | 84.415 | 1.00 | 54.74 | A | C |
| ATOM | 1105 | O | TYR | A | 153 | 38.664 | 43.587 | 84.028 | 1.00 | 55.79 | A | O |
| ATOM | 1106 | N | PHE | A | 154 | 38.605 | 41.579 | 85.066 | 1.00 | 50.96 | A | N |
| ATOM | 1107 | CA | PHE | A | 154 | 40.025 | 41.569 | 85.420 | 1.00 | 48.23 | A | C |
| ATOM | 1108 | CB | PHE | A | 154 | 40.331 | 40.425 | 86.407 | 1.00 | 50.09 | A | C |
| ATOM | 1109 | CG | PHE | A | 154 | 41.810 | 40.159 | 86.596 | 1.00 | 50.17 | A | C |
| ATOM | 1110 | CD1 | PHE | A | 154 | 42.422 | 39.077 | 85.974 | 1.00 | 50.56 | A | C |
| ATOM | 1111 | CD2 | PHE | A | 154 | 42.598 | 41.027 | 87.355 | 1.00 | 48.57 | A | C |
| ATOM | 1112 | CE1 | PHE | A | 154 | 43.803 | 38.864 | 86.097 | 1.00 | 51.73 | A | C |
| ATOM | 1113 | CE2 | PHE | A | 154 | 43.970 | 40.827 | 87.485 | 1.00 | 49.11 | A | C |
| ATOM | 1114 | CZ | PHE | A | 154 | 44.578 | 39.743 | 86.853 | 1.00 | 50.55 | A | C |
| ATOM | 1115 | C | PHE | A | 154 | 40.976 | 41.468 | 84.237 | 1.00 | 46.76 | A | C |
| ATOM | 1116 | O | PHE | A | 154 | 41.929 | 42.249 | 84.127 | 1.00 | 45.78 | A | O |
| ATOM | 1117 | N | LEU | A | 155 | 40.735 | 40.497 | 83.360 | 1.00 | 45.02 | A | N |
| ATOM | 1118 | CA | LEU | A | 155 | 41.599 | 40.313 | 82.205 | 1.00 | 42.62 | A | C |
| ATOM | 1119 | CB | LEU | A | 155 | 41.193 | 39.043 | 81.448 | 1.00 | 44.29 | A | C |
| ATOM | 1120 | CG | LEU | A | 155 | 41.592 | 37.727 | 82.153 | 1.00 | 46.47 | A | C |
| ATOM | 1121 | CD1 | LEU | A | 155 | 41.286 | 36.555 | 81.247 | 1.00 | 44.05 | A | C |
| ATOM | 1122 | CD2 | LEU | A | 155 | 43.090 | 37.735 | 82.496 | 1.00 | 43.43 | A | C |
| ATOM | 1123 | C | LEU | A | 155 | 41.609 | 41.542 | 81.287 | 1.00 | 40.09 | A | C |
| ATOM | 1124 | O | LEU | A | 155 | 42.668 | 41.932 | 80.780 | 1.00 | 40.00 | A | O |
| ATOM | 1125 | N | ASP | A | 156 | 40.454 | 42.168 | 81.080 | 1.00 | 38.07 | A | N |
| ATOM | 1126 | CA | ASP | A | 156 | 40.424 | 43.364 | 80.238 | 1.00 | 41.46 | A | C |
| ATOM | 1127 | CB | ASP | A | 156 | 39.009 | 43.960 | 80.154 | 1.00 | 41.43 | A | C |
| ATOM | 1128 | CG | ASP | A | 156 | 38.127 | 43.265 | 79.122 | 1.00 | 42.27 | A | C |
| ATOM | 1129 | OD1 | ASP | A | 156 | 38.663 | 42.679 | 78.156 | 1.00 | 42.97 | A | O |
| ATOM | 1130 | OD2 | ASP | A | 156 | 36.884 | 43.327 | 79.264 | 1.00 | 46.66 | A | O |
| ATOM | 1131 | C | ASP | A | 156 | 41.379 | 44.410 | 80.832 | 1.00 | 40.47 | A | C |
| ATOM | 1132 | O | ASP | A | 156 | 42.242 | 44.945 | 80.128 | 1.00 | 39.78 | A | O |
| ATOM | 1133 | N | ARG | A | 157 | 41.240 | 44.685 | 82.129 | 1.00 | 40.90 | A | N |
| ATOM | 1134 | CA | ARG | A | 157 | 42.103 | 45.668 | 82.789 | 1.00 | 42.27 | A | C |
| ATOM | 1135 | CB | ARG | A | 157 | 41.621 | 45.912 | 84.219 | 1.00 | 44.37 | A | C |
| ATOM | 1136 | CG | ARG | A | 157 | 40.192 | 46.461 | 84.301 | 1.00 | 47.93 | A | C |
| ATOM | 1137 | CD | ARG | A | 157 | 39.622 | 46.345 | 85.713 | 1.00 | 53.64 | A | C |
| ATOM | 1138 | NE | ARG | A | 157 | 40.372 | 47.145 | 86.674 | 1.00 | 56.20 | A | N |
| ATOM | 1139 | CZ | ARG | A | 157 | 40.265 | 48.465 | 86.780 | 1.00 | 59.70 | A | C |
| ATOM | 1140 | NH1 | ARG | A | 157 | 39.429 | 49.128 | 85.983 | 1.00 | 59.22 | A | N |
| ATOM | 1141 | NH2 | ARG | A | 157 | 41.004 | 49.122 | 87.667 | 1.00 | 59.98 | A | N |
| ATOM | 1142 | C | ARG | A | 157 | 43.564 | 45.242 | 82.800 | 1.00 | 42.74 | A | C |
| ATOM | 1143 | O | ARG | A | 157 | 44.445 | 46.033 | 82.442 | 1.00 | 43.34 | A | O |
| ATOM | 1144 | N | PHE | A | 158 | 43.818 | 43.991 | 83.190 | 1.00 | 44.28 | A | N |
| ATOM | 1145 | CA | PHE | A | 158 | 45.175 | 43.463 | 83.270 | 1.00 | 42.46 | A | C |
| ATOM | 1146 | CB | PHE | A | 158 | 45.153 | 41.974 | 83.655 | 1.00 | 45.03 | A | C |
| ATOM | 1147 | CG | PHE | A | 158 | 46.525 | 41.340 | 83.729 | 1.00 | 48.63 | A | C |
| ATOM | 1148 | CD1 | PHE | A | 158 | 47.409 | 41.667 | 84.758 | 1.00 | 51.42 | A | C |
| ATOM | 1149 | CD2 | PHE | A | 158 | 46.944 | 40.439 | 82.757 | 1.00 | 49.12 | A | C |
| ATOM | 1150 | CE1 | PHE | A | 158 | 48.692 | 41.109 | 84.813 | 1.00 | 49.44 | A | C |
| ATOM | 1151 | CE2 | PHE | A | 158 | 48.221 | 39.879 | 82.803 | 1.00 | 50.35 | A | C |
| ATOM | 1152 | CZ | PHE | A | 158 | 49.097 | 40.216 | 83.836 | 1.00 | 50.91 | A | C |
| ATOM | 1153 | C | PHE | A | 158 | 45.880 | 43.624 | 81.942 | 1.00 | 43.26 | A | C |
| ATOM | 1154 | O | PHE | A | 158 | 46.976 | 44.202 | 81.865 | 1.00 | 41.93 | A | O |
| ATOM | 1155 | N | TYR | A | 159 | 45.237 | 43.120 | 80.890 | 1.00 | 43.08 | A | N |
| ATOM | 1156 | CA | TYR | A | 159 | 45.806 | 43.183 | 79.554 | 1.00 | 41.84 | A | C |
| ATOM | 1157 | CB | TYR | A | 159 | 44.942 | 42.392 | 78.573 | 1.00 | 46.23 | A | C |
| ATOM | 1158 | CG | TYR | A | 159 | 45.083 | 40.899 | 78.724 | 1.00 | 49.37 | A | C |
| ATOM | 1159 | CD1 | TYR | A | 159 | 46.345 | 40.310 | 78.835 | 1.00 | 53.51 | A | C |
| ATOM | 1160 | CE1 | TYR | A | 159 | 46.488 | 38.932 | 78.945 | 1.00 | 55.53 | A | C |
| ATOM | 1161 | CD2 | TYR | A | 159 | 43.964 | 40.066 | 78.730 | 1.00 | 52.10 | A | C |
| ATOM | 1162 | CE2 | TYR | A | 159 | 44.099 | 38.685 | 78.839 | 1.00 | 53.20 | A | C |
| ATOM | 1163 | CZ | TYR | A | 159 | 45.362 | 38.127 | 78.944 | 1.00 | 54.15 | A | C |
| ATOM | 1164 | OH | TYR | A | 159 | 45.498 | 36.761 | 79.029 | 1.00 | 56.83 | A | O |
| ATOM | 1165 | C | TYR | A | 159 | 45.999 | 44.607 | 79.065 | 1.00 | 40.26 | A | C |
| ATOM | 1166 | O | TYR | A | 159 | 47.072 | 44.927 | 78.536 | 1.00 | 39.51 | A | O |
| ATOM | 1167 | N | LEU | A | 160 | 44.989 | 45.466 | 79.242 | 1.00 | 40.65 | A | N |
| ATOM | 1168 | CA | LEU | A | 160 | 45.125 | 46.871 | 78.808 | 1.00 | 42.91 | A | C |
| ATOM | 1169 | CB | LEU | A | 160 | 43.892 | 47.688 | 79.184 | 1.00 | 43.33 | A | C |
| ATOM | 1170 | CG | LEU | A | 160 | 43.250 | 48.523 | 78.063 | 1.00 | 46.57 | A | C |
| ATOM | 1171 | CD1 | LEU | A | 160 | 42.347 | 49.564 | 78.696 | 1.00 | 41.64 | A | C |
| ATOM | 1172 | CD2 | LEU | A | 160 | 44.310 | 49.189 | 77.181 | 1.00 | 43.52 | A | C |
| ATOM | 1173 | C | LEU | A | 160 | 46.349 | 47.451 | 79.518 | 1.00 | 42.08 | A | C |
| ATOM | 1174 | O | LEU | A | 160 | 47.232 | 48.049 | 78.887 | 1.00 | 43.55 | A | O |
| ATOM | 1175 | N | SER | A | 161 | 46.402 | 47.248 | 80.832 | 1.00 | 43.26 | A | N |
| ATOM | 1176 | CA | SER | A | 161 | 47.525 | 47.710 | 81.638 | 1.00 | 44.72 | A | C |
| ATOM | 1177 | CB | SER | A | 161 | 47.425 | 47.123 | 83.049 | 1.00 | 49.37 | A | C |
| ATOM | 1178 | OG | SER | A | 161 | 48.644 | 47.273 | 83.764 | 1.00 | 58.76 | A | O |
| ATOM | 1179 | C | SER | A | 161 | 48.815 | 47.249 | 80.964 | 1.00 | 42.91 | A | C |
| ATOM | 1180 | O | SER | A | 161 | 49.716 | 48.064 | 80.721 | 1.00 | 40.16 | A | O |
| ATOM | 1181 | N | ARG | A | 162 | 48.899 | 45.954 | 80.637 | 1.00 | 39.39 | A | N |
| ATOM | 1182 | CA | ARG | A | 162 | 50.116 | 45.432 | 80.002 | 1.00 | 38.06 | A | C |
| ATOM | 1183 | CB | ARG | A | 162 | 50.003 | 43.915 | 79.739 | 1.00 | 37.13 | A | C |
| ATOM | 1184 | CG | ARG | A | 162 | 51.210 | 43.319 | 78.996 | 1.00 | 34.59 | A | C |
| ATOM | 1185 | CD | ARG | A | 162 | 51.110 | 41.787 | 78.909 | 1.00 | 40.82 | A | C |
| ATOM | 1186 | NE | ARG | A | 162 | 52.304 | 41.199 | 78.297 | 1.00 | 43.75 | A | N |
| ATOM | 1187 | CZ | ARG | A | 162 | 52.365 | 40.783 | 77.038 | 1.00 | 44.19 | A | C |
| ATOM | 1188 | NH1 | ARG | A | 162 | 51.294 | 40.893 | 76.270 | 1.00 | 46.45 | A | N |
| ATOM | 1189 | NH2 | ARG | A | 162 | 53.486 | 40.264 | 76.548 | 1.00 | 46.05 | A | N |
| ATOM | 1190 | C | ARG | A | 162 | 50.434 | 46.159 | 78.702 | 1.00 | 36.50 | A | C |
| ATOM | 1191 | O | ARG | A | 162 | 51.585 | 46.537 | 78.452 | 1.00 | 34.91 | A | O |
| ATOM | 1192 | N | ILE | A | 163 | 49.418 | 46.339 | 77.862 | 1.00 | 37.08 | A | N |
| ATOM | 1193 | CA | ILE | A | 163 | 49.605 | 47.043 | 76.597 | 1.00 | 37.47 | A | C |
| ATOM | 1194 | CB | ILE | A | 163 | 48.260 | 47.238 | 75.869 | 1.00 | 38.60 | A | C |
| ATOM | 1195 | CG2 | ILE | A | 163 | 48.429 | 48.211 | 74.700 | 1.00 | 37.67 | A | C |
| ATOM | 1196 | CG1 | ILE | A | 163 | 47.750 | 45.886 | 75.372 | 1.00 | 40.21 | A | C |
| ATOM | 1197 | CD1 | ILE | A | 163 | 46.351 | 45.929 | 74.819 | 1.00 | 43.34 | A | C |
| ATOM | 1198 | C | ILE | A | 163 | 50.235 | 48.417 | 76.834 | 1.00 | 36.58 | A | C |
| ATOM | 1199 | O | ILE | A | 163 | 51.195 | 48.782 | 76.158 | 1.00 | 38.82 | A | O |
| ATOM | 1200 | N | SER | A | 164 | 49.700 | 49.181 | 77.784 | 1.00 | 36.49 | A | N |
| ATOM | 1201 | CA | SER | A | 164 | 50.232 | 50.523 | 78.077 | 1.00 | 40.71 | A | C |
| ATOM | 1202 | CB | SER | A | 164 | 49.391 | 51.208 | 79.143 | 1.00 | 39.36 | A | C |
| ATOM | 1203 | OG | SER | A | 164 | 49.642 | 50.634 | 80.408 | 1.00 | 47.01 | A | O |
| ATOM | 1204 | C | SER | A | 164 | 51.686 | 50.486 | 78.553 | 1.00 | 42.00 | A | C |
| ATOM | 1205 | O | SER | A | 164 | 52.490 | 51.376 | 78.206 | 1.00 | 40.70 | A | O |
| ATOM | 1206 | N | ILE | A | 165 | 52.033 | 49.460 | 79.338 | 1.00 | 40.82 | A | N |
| ATOM | 1207 | CA | ILE | A | 165 | 53.403 | 49.341 | 79.834 | 1.00 | 42.12 | A | C |
| ATOM | 1208 | CB | ILE | A | 165 | 53.528 | 48.250 | 80.904 | 1.00 | 43.75 | A | C |
| ATOM | 1209 | CG2 | ILE | A | 165 | 54.995 | 47.960 | 81.198 | 1.00 | 44.35 | A | C |
| ATOM | 1210 | CG1 | ILE | A | 165 | 52.812 | 48.705 | 82.173 | 1.00 | 41.63 | A | C |
| ATOM | 1211 | CD1 | ILE | A | 165 | 52.674 | 47.639 | 83.215 | 1.00 | 45.20 | A | C |
| ATOM | 1212 | C | ILE | A | 165 | 54.324 | 49.025 | 78.684 | 1.00 | 42.18 | A | C |
| ATOM | 1213 | O | ILE | A | 165 | 55.386 | 49.635 | 78.553 | 1.00 | 41.54 | A | O |
| ATOM | 1214 | N | ARG | A | 166 | 53.915 | 48.082 | 77.840 | 1.00 | 44.00 | A | N |
| ATOM | 1215 | CA | ARG | A | 166 | 54.728 | 47.709 | 76.690 | 1.00 | 45.50 | A | C |
| ATOM | 1216 | CB | ARG | A | 166 | 54.107 | 46.496 | 75.980 | 1.00 | 48.41 | A | C |
| ATOM | 1217 | CG | ARG | A | 166 | 54.474 | 45.169 | 76.671 | 1.00 | 55.80 | A | C |
| ATOM | 1218 | CD | ARG | A | 166 | 53.660 | 43.988 | 76.186 | 1.00 | 57.95 | A | C |
| ATOM | 1219 | NE | ARG | A | 166 | 53.835 | 43.765 | 74.759 | 1.00 | 65.33 | A | N |
| ATOM | 1220 | CZ | ARG | A | 166 | 54.940 | 43.281 | 74.206 | 1.00 | 69.48 | A | C |
| ATOM | 1221 | NH1 | ARG | A | 166 | 55.983 | 42.957 | 74.969 | 1.00 | 68.83 | A | N |
| ATOM | 1222 | NH2 | ARG | A | 166 | 54.998 | 43.130 | 72.885 | 1.00 | 70.43 | A | N |
| ATOM | 1223 | C | ARG | A | 166 | 54.910 | 48.885 | 75.734 | 1.00 | 45.25 | A | C |
| ATOM | 1224 | O | ARG | A | 166 | 55.964 | 49.025 | 75.106 | 1.00 | 44.44 | A | O |
| ATOM | 1225 | N | MET | A | 167 | 53.892 | 49.739 | 75.640 | 1.00 | 45.20 | A | N |
| ATOM | 1226 | CA | MET | A | 167 | 53.962 | 50.916 | 74.773 | 1.00 | 47.15 | A | C |
| ATOM | 1227 | CB | MET | A | 167 | 52.653 | 51.723 | 74.857 | 1.00 | 50.76 | A | C |
| ATOM | 1228 | CG | MET | A | 167 | 51.941 | 52.001 | 73.522 | 1.00 | 52.22 | A | C |
| ATOM | 1229 | SD | MET | A | 167 | 52.893 | 52.834 | 72.230 | 1.00 | 50.12 | A | S |
| ATOM | 1230 | CE | MET | A | 167 | 52.241 | 54.472 | 72.289 | 1.00 | 53.09 | A | C |
| ATOM | 1231 | C | MET | A | 167 | 55.126 | 51.807 | 75.228 | 1.00 | 45.76 | A | C |
| ATOM | 1232 | O | MET | A | 167 | 56.006 | 52.164 | 74.439 | 1.00 | 47.00 | A | O |
| ATOM | 1233 | N | LEU | A | 168 | 55.127 | 52.172 | 76.502 | 1.00 | 44.99 | A | N |
| ATOM | 1234 | CA | LEU | A | 168 | 56.198 | 53.019 | 77.017 | 1.00 | 47.25 | A | C |
| ATOM | 1235 | CB | LEU | A | 168 | 56.026 | 53.235 | 78.523 | 1.00 | 45.77 | A | C |
| ATOM | 1236 | CG | LEU | A | 168 | 54.677 | 53.832 | 78.936 | 1.00 | 47.27 | A | C |
| ATOM | 1237 | CD1 | LEU | A | 168 | 54.743 | 54.337 | 80.380 | 1.00 | 43.07 | A | C |
| ATOM | 1238 | CD2 | LEU | A | 168 | 54.337 | 54.974 | 77.985 | 1.00 | 44.59 | A | C |
| ATOM | 1239 | C | LEU | A | 168 | 57.549 | 52.380 | 76.731 | 1.00 | 47.95 | A | C |
| ATOM | 1240 | O | LEU | A | 168 | 58.440 | 53.007 | 76.158 | 1.00 | 50.29 | A | O |
| ATOM | 1241 | N | ILE | A | 169 | 57.700 | 51.122 | 77.118 | 1.00 | 46.99 | A | N |
| ATOM | 1242 | CA | ILE | A | 169 | 58.955 | 50.422 | 76.893 | 1.00 | 48.47 | A | C |
| ATOM | 1243 | CB | ILE | A | 169 | 58.874 | 48.970 | 77.388 | 1.00 | 48.49 | A | C |
| ATOM | 1244 | CG2 | ILE | A | 169 | 60.099 | 48.193 | 76.927 | 1.00 | 46.62 | A | C |
| ATOM | 1245 | CG1 | ILE | A | 169 | 58.736 | 48.956 | 78.911 | 1.00 | 51.25 | A | C |
| ATOM | 1246 | CD1 | ILE | A | 169 | 58.595 | 47.570 | 79.515 | 1.00 | 51.53 | A | C |
| ATOM | 1247 | C | ILE | A | 169 | 59.403 | 50.400 | 75.432 | 1.00 | 48.95 | A | C |
| ATOM | 1248 | O | ILE | A | 169 | 60.581 | 50.598 | 75.138 | 1.00 | 47.83 | A | O |
| ATOM | 1249 | N | ASN | A | 170 | 58.479 | 50.143 | 74.515 | 1.00 | 50.53 | A | N |
| ATOM | 1250 | CA | ASN | A | 170 | 58.849 | 50.094 | 73.108 | 1.00 | 53.15 | A | C |
| ATOM | 1251 | CB | ASN | A | 170 | 57.707 | 49.538 | 72.261 | 1.00 | 56.42 | A | C |
| ATOM | 1252 | CG | ASN | A | 170 | 57.449 | 48.074 | 72.526 | 1.00 | 58.97 | A | C |
| ATOM | 1253 | OD1 | ASN | A | 170 | 58.384 | 47.276 | 72.631 | 1.00 | 62.38 | A | O |
| ATOM | 1254 | ND2 | ASN | A | 170 | 56.177 | 47.706 | 72.620 | 1.00 | 60.16 | A | N |
| ATOM | 1255 | C | ASN | A | 170 | 59.216 | 51.471 | 72.600 | 1.00 | 53.92 | A | C |
| ATOM | 1256 | O | ASN | A | 170 | 60.063 | 51.612 | 71.716 | 1.00 | 53.31 | A | O |
| ATOM | 1257 | N | GLN | A | 171 | 58.558 | 52.485 | 73.150 | 1.00 | 53.20 | A | N |
| ATOM | 1258 | CA | GLN | A | 171 | 58.830 | 53.849 | 72.747 | 1.00 | 56.25 | A | C |
| ATOM | 1259 | CB | GLN | A | 171 | 57.796 | 54.798 | 73.365 | 1.00 | 55.86 | A | C |
| ATOM | 1260 | CG | GLN | A | 171 | 56.534 | 54.975 | 72.521 | 1.00 | 55.36 | A | C |
| ATOM | 1261 | CD | GLN | A | 171 | 56.851 | 55.404 | 71.098 | 1.00 | 56.58 | A | C |
| ATOM | 1262 | OE1 | GLN | A | 171 | 56.966 | 54.570 | 70.189 | 1.00 | 54.87 | A | O |
| ATOM | 1263 | NE2 | GLN | A | 171 | 57.017 | 56.711 | 70.898 | 1.00 | 53.97 | A | N |
| ATOM | 1264 | C | GLN | A | 171 | 60.245 | 54.241 | 73.164 | 1.00 | 58.37 | A | C |
| ATOM | 1265 | O | GLN | A | 171 | 60.984 | 54.842 | 72.387 | 1.00 | 57.72 | A | O |
| ATOM | 1266 | N | HIS | A | 172 | 60.632 | 53.873 | 74.380 | 1.00 | 60.86 | A | N |
| ATOM | 1267 | CA | HIS | A | 172 | 61.962 | 54.196 | 74.883 | 1.00 | 64.11 | A | C |
| ATOM | 1268 | CB | HIS | A | 172 | 62.046 | 53.883 | 76.375 | 1.00 | 67.56 | A | C |
| ATOM | 1269 | CG | HIS | A | 172 | 63.264 | 54.441 | 77.036 | 1.00 | 73.79 | A | C |
| ATOM | 1270 | CD2 | HIS | A | 172 | 64.431 | 53.855 | 77.395 | 1.00 | 75.91 | A | C |
| ATOM | 1271 | ND1 | HIS | A | 172 | 63.389 | 55.775 | 77.361 | 1.00 | 76.41 | A | N |
| ATOM | 1272 | CE1 | HIS | A | 172 | 64.580 | 55.986 | 77.891 | 1.00 | 77.16 | A | C |
| ATOM | 1273 | NE2 | HIS | A | 172 | 65.232 | 54.838 | 77.922 | 1.00 | 78.06 | A | N |
| ATOM | 1274 | C | HIS | A | 172 | 63.055 | 53.424 | 74.142 | 1.00 | 64.68 | A | C |
| ATOM | 1275 | O | HIS | A | 172 | 63.945 | 54.016 | 73.522 | 1.00 | 64.90 | A | O |
| ATOM | 1276 | N | THR | A | 173 | 62.977 | 52.099 | 74.207 | 1.00 | 63.60 | A | N |
| ATOM | 1277 | CA | THR | A | 173 | 63.960 | 51.230 | 73.572 | 1.00 | 64.66 | A | C |
| ATOM | 1278 | CB | THR | A | 173 | 63.574 | 49.755 | 73.759 | 1.00 | 64.92 | A | C |
| ATOM | 1279 | OG1 | THR | A | 173 | 62.282 | 49.524 | 73.185 | 1.00 | 65.64 | A | O |
| ATOM | 1280 | CG2 | THR | A | 173 | 63.522 | 49.412 | 75.238 | 1.00 | 63.30 | A | C |
| ATOM | 1281 | C | THR | A | 173 | 64.165 | 51.505 | 72.084 | 1.00 | 65.65 | A | C |
| ATOM | 1282 | O | THR | A | 173 | 65.298 | 51.514 | 71.602 | 1.00 | 64.72 | A | O |
| ATOM | 1283 | N | LEU | A | 174 | 63.071 | 51.723 | 71.360 | 1.00 | 67.16 | A | N |
| ATOM | 1284 | CA | LEU | A | 174 | 63.143 | 52.001 | 69.930 | 1.00 | 68.23 | A | C |
| ATOM | 1285 | CB | LEU | A | 174 | 61.755 | 51.894 | 69.294 | 1.00 | 68.23 | A | C |
| ATOM | 1286 | CG | LEU | A | 174 | 61.258 | 50.542 | 68.778 | 1.00 | 66.30 | A | C |
| ATOM | 1287 | CD1 | LEU | A | 174 | 61.078 | 49.558 | 69.915 | 1.00 | 67.15 | A | C |
| ATOM | 1288 | CD2 | LEU | A | 174 | 59.939 | 50.766 | 68.059 | 1.00 | 66.80 | A | C |
| ATOM | 1289 | C | LEU | A | 174 | 63.718 | 53.383 | 69.632 | 1.00 | 69.81 | A | C |
| ATOM | 1290 | O | LEU | A | 174 | 64.488 | 53.552 | 68.685 | 1.00 | 69.95 | A | O |
| ATOM | 1291 | N | ILE | A | 175 | 63.330 | 54.370 | 70.435 | 1.00 | 72.26 | A | N |
| ATOM | 1292 | CA | ILE | A | 175 | 63.801 | 55.744 | 70.261 | 1.00 | 74.36 | A | C |
| ATOM | 1293 | CB | ILE | A | 175 | 63.023 | 56.723 | 71.182 | 1.00 | 73.44 | A | C |
| ATOM | 1294 | CG2 | ILE | A | 175 | 63.740 | 58.068 | 71.257 | 1.00 | 73.34 | A | C |
| ATOM | 1295 | CG1 | ILE | A | 175 | 61.598 | 56.922 | 70.660 | 1.00 | 72.72 | A | C |
| ATOM | 1296 | CD1 | ILE | A | 175 | 60.732 | 57.771 | 71.566 | 1.00 | 69.08 | A | C |
| ATOM | 1297 | C | ILE | A | 175 | 65.293 | 55.915 | 70.553 | 1.00 | 76.26 | A | C |
| ATOM | 1298 | O | ILE | A | 175 | 65.974 | 56.686 | 69.873 | 1.00 | 77.08 | A | O |
| ATOM | 1299 | N | PHE | A | 176 | 65.801 | 55.189 | 71.546 | 1.00 | 77.37 | A | N |
| ATOM | 1300 | CA | PHE | A | 176 | 67.200 | 55.317 | 71.935 | 1.00 | 79.28 | A | C |
| ATOM | 1301 | CB | PHE | A | 176 | 67.260 | 55.612 | 73.436 | 1.00 | 75.25 | A | C |
| ATOM | 1302 | CG | PHE | A | 176 | 66.455 | 56.820 | 73.838 | 1.00 | 71.47 | A | C |
| ATOM | 1303 | CD1 | PHE | A | 176 | 66.773 | 58.084 | 73.339 | 1.00 | 69.51 | A | C |
| ATOM | 1304 | CD2 | PHE | A | 176 | 65.350 | 56.692 | 74.671 | 1.00 | 70.01 | A | C |
| ATOM | 1305 | CE1 | PHE | A | 176 | 65.996 | 59.203 | 73.660 | 1.00 | 68.31 | A | C |
| ATOM | 1306 | CE2 | PHE | A | 176 | 64.562 | 57.803 | 75.000 | 1.00 | 67.95 | A | C |
| ATOM | 1307 | CZ | PHE | A | 176 | 64.887 | 59.060 | 74.492 | 1.00 | 68.11 | A | C |
| ATOM | 1308 | C | PHE | A | 176 | 68.129 | 54.153 | 71.584 | 1.00 | 82.79 | A | C |
| ATOM | 1309 | O | PHE | A | 176 | 69.237 | 54.063 | 72.112 | 1.00 | 83.93 | A | O |
| ATOM | 1310 | N | ASP | A | 177 | 67.679 | 53.284 | 70.682 | 1.00 | 86.98 | A | N |
| ATOM | 1311 | CA | ASP | A | 177 | 68.452 | 52.124 | 70.231 | 1.00 | 90.29 | A | C |
| ATOM | 1312 | CB | ASP | A | 177 | 68.366 | 52.012 | 68.706 | 1.00 | 93.56 | A | C |
| ATOM | 1313 | CG | ASP | A | 177 | 68.710 | 53.314 | 68.004 | 1.00 | 97.30 | A | C |
| ATOM | 1314 | OD1 | ASP | A | 177 | 69.833 | 53.830 | 68.202 | 1.00 | 99.45 | A | O |
| ATOM | 1315 | OD2 | ASP | A | 177 | 67.852 | 53.824 | 67.252 | 1.00 | 98.96 | A | O |
| ATOM | 1316 | C | ASP | A | 177 | 69.920 | 52.145 | 70.657 | 1.00 | 90.64 | A | C |
| ATOM | 1317 | O | ASP | A | 177 | 70.801 | 52.540 | 69.889 | 1.00 | 90.69 | A | O |
| ATOM | 1318 | N | LYS | A | 186 | 61.394 | 59.584 | 60.601 | 1.00 | 76.06 | A | N |
| ATOM | 1319 | CA | LYS | A | 186 | 60.057 | 59.386 | 60.043 | 1.00 | 75.76 | A | C |
| ATOM | 1320 | CB | LYS | A | 186 | 60.118 | 58.466 | 58.818 | 1.00 | 78.59 | A | C |
| ATOM | 1321 | CG | LYS | A | 186 | 61.139 | 58.879 | 57.763 | 1.00 | 82.99 | A | C |
| ATOM | 1322 | CD | LYS | A | 186 | 62.557 | 58.478 | 58.169 | 1.00 | 85.68 | A | C |
| ATOM | 1323 | CE | LYS | A | 186 | 63.581 | 58.932 | 57.141 | 1.00 | 87.79 | A | C |
| ATOM | 1324 | NZ | LYS | A | 186 | 63.263 | 58.427 | 55.776 | 1.00 | 89.54 | A | N |
| ATOM | 1325 | C | LYS | A | 186 | 59.137 | 58.780 | 61.102 | 1.00 | 72.47 | A | C |
| ATOM | 1326 | O | LYS | A | 186 | 58.036 | 59.266 | 61.334 | 1.00 | 72.23 | A | O |
| ATOM | 1327 | N | HIS | A | 187 | 59.592 | 57.709 | 61.737 | 1.00 | 69.28 | A | N |
| ATOM | 1328 | CA | HIS | A | 187 | 58.805 | 57.070 | 62.781 | 1.00 | 67.06 | A | C |
| ATOM | 1329 | CB | HIS | A | 187 | 58.869 | 55.550 | 62.674 | 1.00 | 67.94 | A | C |
| ATOM | 1330 | CG | HIS | A | 187 | 57.960 | 54.965 | 61.642 | 1.00 | 68.73 | A | C |
| ATOM | 1331 | CD2 | HIS | A | 187 | 56.886 | 54.149 | 61.765 | 1.00 | 67.96 | A | C |
| ATOM | 1332 | ND1 | HIS | A | 187 | 58.146 | 55.148 | 60.288 | 1.00 | 68.85 | A | N |
| ATOM | 1333 | CE1 | HIS | A | 187 | 57.231 | 54.465 | 59.623 | 1.00 | 67.70 | A | C |
| ATOM | 1334 | NE2 | HIS | A | 187 | 56.455 | 53.850 | 60.498 | 1.00 | 68.01 | A | N |
| ATOM | 1335 | C | HIS | A | 187 | 59.366 | 57.467 | 64.130 | 1.00 | 64.72 | A | C |
| ATOM | 1336 | O | HIS | A | 187 | 60.583 | 57.532 | 64.308 | 1.00 | 66.21 | A | O |
| ATOM | 1337 | N | ILE | A | 188 | 58.479 | 57.726 | 65.080 | 1.00 | 59.28 | A | N |
| ATOM | 1338 | CA | ILE | A | 188 | 58.903 | 58.092 | 66.416 | 1.00 | 56.81 | A | C |
| ATOM | 1339 | CB | ILE | A | 188 | 58.222 | 59.403 | 66.863 | 1.00 | 55.47 | A | C |
| ATOM | 1340 | CG2 | ILE | A | 188 | 58.518 | 59.687 | 68.328 | 1.00 | 50.68 | A | C |
| ATOM | 1341 | CG1 | ILE | A | 188 | 58.733 | 60.543 | 65.971 | 1.00 | 57.61 | A | C |
| ATOM | 1342 | CD1 | ILE | A | 188 | 58.217 | 61.927 | 66.327 | 1.00 | 60.00 | A | C |
| ATOM | 1343 | C | ILE | A | 188 | 58.585 | 56.939 | 67.357 | 1.00 | 56.35 | A | C |
| ATOM | 1344 | O | ILE | A | 188 | 57.543 | 56.913 | 68.021 | 1.00 | 55.01 | A | O |
| ATOM | 1345 | N | GLY | A | 189 | 59.507 | 55.982 | 67.403 | 1.00 | 56.67 | A | N |
| ATOM | 1346 | CA | GLY | A | 189 | 59.321 | 54.811 | 68.235 | 1.00 | 54.26 | A | C |
| ATOM | 1347 | C | GLY | A | 189 | 58.479 | 53.832 | 67.445 | 1.00 | 53.70 | A | C |
| ATOM | 1348 | O | GLY | A | 189 | 58.905 | 53.338 | 66.399 | 1.00 | 54.81 | A | O |
| ATOM | 1349 | N | SER | A | 190 | 57.276 | 53.565 | 67.932 | 1.00 | 50.84 | A | N |
| ATOM | 1350 | CA | SER | A | 190 | 56.373 | 52.650 | 67.254 | 1.00 | 50.58 | A | C |
| ATOM | 1351 | CB | SER | A | 190 | 55.739 | 51.698 | 68.263 | 1.00 | 47.99 | A | C |
| ATOM | 1352 | OG | SER | A | 190 | 54.828 | 52.389 | 69.105 | 1.00 | 47.73 | A | O |
| ATOM | 1353 | C | SER | A | 190 | 55.274 | 53.468 | 66.567 | 1.00 | 50.70 | A | C |
| ATOM | 1354 | O | SER | A | 190 | 54.358 | 52.919 | 65.953 | 1.00 | 50.36 | A | O |
| ATOM | 1355 | N | ILE | A | 191 | 55.383 | 54.785 | 66.672 | 1.00 | 50.19 | A | N |
| ATOM | 1356 | CA | ILE | A | 191 | 54.391 | 55.677 | 66.097 | 1.00 | 50.86 | A | C |
| ATOM | 1357 | CB | ILE | A | 191 | 54.138 | 56.867 | 67.034 | 1.00 | 52.29 | A | C |
| ATOM | 1358 | CG2 | ILE | A | 191 | 53.199 | 57.865 | 66.382 | 1.00 | 52.53 | A | C |
| ATOM | 1359 | CG1 | ILE | A | 191 | 53.568 | 56.359 | 68.358 | 1.00 | 48.88 | A | C |
| ATOM | 1360 | CD1 | ILE | A | 191 | 53.354 | 57.444 | 69.373 | 1.00 | 48.16 | A | C |
| ATOM | 1361 | C | ILE | A | 191 | 54.739 | 56.217 | 64.720 | 1.00 | 49.61 | A | C |
| ATOM | 1362 | O | ILE | A | 191 | 55.838 | 56.722 | 64.493 | 1.00 | 53.13 | A | O |
| ATOM | 1363 | N | ASP | A | 192 | 53.793 | 56.088 | 63.795 | 1.00 | 47.04 | A | N |
| ATOM | 1364 | CA | ASP | A | 192 | 53.962 | 56.599 | 62.437 | 1.00 | 45.66 | A | C |
| ATOM | 1365 | CB | ASP | A | 192 | 53.494 | 55.589 | 61.398 | 1.00 | 44.23 | A | C |
| ATOM | 1366 | CG | ASP | A | 192 | 53.850 | 56.002 | 59.985 | 1.00 | 43.30 | A | C |
| ATOM | 1367 | OD1 | ASP | A | 192 | 54.050 | 57.213 | 59.752 | 1.00 | 43.66 | A | O |
| ATOM | 1368 | OD2 | ASP | A | 192 | 53.917 | 55.117 | 59.107 | 1.00 | 40.41 | A | O |
| ATOM | 1369 | C | ASP | A | 192 | 53.061 | 57.822 | 62.382 | 1.00 | 46.89 | A | C |
| ATOM | 1370 | O | ASP | A | 192 | 51.837 | 57.711 | 62.506 | 1.00 | 47.40 | A | O |
| ATOM | 1371 | N | PRO | A | 193 | 53.653 | 59.014 | 62.227 | 1.00 | 48.95 | A | N |
| ATOM | 1372 | CD | PRO | A | 193 | 55.089 | 59.308 | 62.080 | 1.00 | 49.07 | A | C |
| ATOM | 1373 | CA | PRO | A | 193 | 52.861 | 60.244 | 62.170 | 1.00 | 49.07 | A | C |
| ATOM | 1374 | CB | PRO | A | 193 | 53.917 | 61.332 | 62.345 | 1.00 | 48.13 | A | C |
| ATOM | 1375 | CG | PRO | A | 193 | 55.081 | 60.756 | 61.609 | 1.00 | 49.79 | A | C |
| ATOM | 1376 | C | PRO | A | 193 | 52.092 | 60.366 | 60.860 | 1.00 | 48.33 | A | C |
| ATOM | 1377 | O | PRO | A | 193 | 51.154 | 61.148 | 60.761 | 1.00 | 49.48 | A | O |
| ATOM | 1378 | N | ASN | A | 194 | 52.500 | 59.588 | 59.862 | 1.00 | 49.10 | A | N |
| ATOM | 1379 | CA | ASN | A | 194 | 51.846 | 59.592 | 58.554 | 1.00 | 51.98 | A | C |
| ATOM | 1380 | CB | ASN | A | 194 | 52.799 | 60.144 | 57.484 | 1.00 | 57.42 | A | C |
| ATOM | 1381 | CG | ASN | A | 194 | 52.857 | 61.664 | 57.483 | 1.00 | 65.16 | A | C |
| ATOM | 1382 | OD1 | ASN | A | 194 | 53.203 | 62.285 | 58.488 | 1.00 | 68.18 | A | O |
| ATOM | 1383 | ND2 | ASN | A | 194 | 52.513 | 62.270 | 56.350 | 1.00 | 69.51 | A | N |
| ATOM | 1384 | C | ASN | A | 194 | 51.378 | 58.193 | 58.152 | 1.00 | 49.22 | A | C |
| ATOM | 1385 | O | ASN | A | 194 | 51.563 | 57.771 | 57.008 | 1.00 | 48.83 | A | O |
| ATOM | 1386 | N | CYS | A | 195 | 50.762 | 57.488 | 59.095 | 1.00 | 45.38 | A | N |
| ATOM | 1387 | CA | CYS | A | 195 | 50.279 | 56.135 | 58.851 | 1.00 | 44.18 | A | C |
| ATOM | 1388 | CB | CYS | A | 195 | 49.704 | 55.551 | 60.139 | 1.00 | 42.37 | A | C |
| ATOM | 1389 | SG | CYS | A | 195 | 49.143 | 53.851 | 59.946 | 1.00 | 42.58 | A | S |
| ATOM | 1390 | C | CYS | A | 195 | 49.215 | 56.072 | 57.767 | 1.00 | 41.61 | A | C |
| ATOM | 1391 | O | CYS | A | 195 | 48.144 | 56.664 | 57.924 | 1.00 | 43.97 | A | O |
| ATOM | 1392 | N | ASN | A | 196 | 49.507 | 55.367 | 56.673 | 1.00 | 40.45 | A | N |
| ATOM | 1393 | CA | ASN | A | 196 | 48.541 | 55.217 | 55.585 | 1.00 | 41.47 | A | C |
| ATOM | 1394 | CB | ASN | A | 196 | 49.243 | 55.063 | 54.233 | 1.00 | 41.95 | A | C |
| ATOM | 1395 | CG | ASN | A | 196 | 48.254 | 54.871 | 53.094 | 1.00 | 45.90 | A | C |
| ATOM | 1396 | OD1 | ASN | A | 196 | 47.539 | 53.875 | 53.046 | 1.00 | 48.55 | A | O |
| ATOM | 1397 | ND2 | ASN | A | 196 | 48.195 | 55.833 | 52.187 | 1.00 | 50.62 | A | N |
| ATOM | 1398 | C | ASN | A | 196 | 47.721 | 53.963 | 55.890 | 1.00 | 41.20 | A | C |
| ATOM | 1399 | O | ASN | A | 196 | 48.143 | 52.832 | 55.612 | 1.00 | 41.59 | A | O |
| ATOM | 1400 | N | VAL | A | 197 | 46.547 | 54.170 | 56.467 | 1.00 | 38.40 | A | N |
| ATOM | 1401 | CA | VAL | A | 197 | 45.693 | 53.062 | 56.866 | 1.00 | 36.28 | A | C |
| ATOM | 1402 | CB | VAL | A | 197 | 44.333 | 53.578 | 57.320 | 1.00 | 32.88 | A | C |
| ATOM | 1403 | CG1 | VAL | A | 197 | 43.457 | 52.412 | 57.765 | 1.00 | 33.31 | A | C |
| ATOM | 1404 | CG2 | VAL | A | 197 | 44.518 | 54.569 | 58.456 | 1.00 | 31.67 | A | C |
| ATOM | 1405 | C | VAL | A | 197 | 45.485 | 51.994 | 55.806 | 1.00 | 37.67 | A | C |
| ATOM | 1406 | O | VAL | A | 197 | 45.657 | 50.799 | 56.082 | 1.00 | 38.70 | A | O |
| ATOM | 1407 | N | SER | A | 198 | 45.113 | 52.402 | 54.599 | 1.00 | 37.94 | A | N |
| ATOM | 1408 | CA | SER | A | 198 | 44.867 | 51.422 | 53.550 | 1.00 | 41.04 | A | C |
| ATOM | 1409 | CB | SER | A | 198 | 44.460 | 52.107 | 52.250 | 1.00 | 40.99 | A | C |
| ATOM | 1410 | OG | SER | A | 198 | 43.077 | 52.404 | 52.270 | 1.00 | 45.02 | A | O |
| ATOM | 1411 | C | SER | A | 198 | 46.052 | 50.524 | 53.294 | 1.00 | 42.95 | A | C |
| ATOM | 1412 | O | SER | A | 198 | 45.883 | 49.374 | 52.883 | 1.00 | 44.84 | A | O |
| ATOM | 1413 | N | GLU | A | 199 | 47.251 | 51.040 | 53.543 | 1.00 | 44.51 | A | N |
| ATOM | 1414 | CA | GLU | A | 199 | 48.463 | 50.265 | 53.324 | 1.00 | 46.17 | A | C |
| ATOM | 1415 | CB | GLU | A | 199 | 49.695 | 51.184 | 53.323 | 1.00 | 50.99 | A | C |
| ATOM | 1416 | CG | GLU | A | 199 | 51.027 | 50.475 | 53.035 | 1.00 | 59.71 | A | C |
| ATOM | 1417 | CD | GLU | A | 199 | 50.980 | 49.582 | 51.791 | 1.00 | 64.65 | A | C |
| ATOM | 1418 | OE1 | GLU | A | 199 | 50.360 | 49.993 | 50.779 | 1.00 | 67.70 | A | O |
| ATOM | 1419 | OE2 | GLU | A | 199 | 51.571 | 48.474 | 51.823 | 1.00 | 65.63 | A | O |
| ATOM | 1420 | C | GLU | A | 199 | 48.572 | 49.214 | 54.415 | 1.00 | 43.76 | A | C |
| ATOM | 1421 | O | GLU | A | 199 | 48.974 | 48.069 | 54.150 | 1.00 | 43.64 | A | O |
| ATOM | 1422 | N | VAL | A | 200 | 48.219 | 49.582 | 55.644 | 1.00 | 38.30 | A | N |
| ATOM | 1423 | CA | VAL | A | 200 | 48.292 | 48.596 | 56.710 | 1.00 | 39.12 | A | C |
| ATOM | 1424 | CB | VAL | A | 200 | 48.059 | 49.198 | 58.105 | 1.00 | 37.95 | A | C |
| ATOM | 1425 | CG1 | VAL | A | 200 | 48.191 | 48.087 | 59.152 | 1.00 | 36.41 | A | C |
| ATOM | 1426 | CG2 | VAL | A | 200 | 49.062 | 50.315 | 58.395 | 1.00 | 35.62 | A | C |
| ATOM | 1427 | C | VAL | A | 200 | 47.215 | 47.545 | 56.448 | 1.00 | 41.47 | A | C |
| ATOM | 1428 | O | VAL | A | 200 | 47.407 | 46.359 | 56.748 | 1.00 | 41.79 | A | O |
| ATOM | 1429 | N | VAL | A | 201 | 46.089 | 47.978 | 55.870 | 1.00 | 39.06 | A | N |
| ATOM | 1430 | CA | VAL | A | 201 | 45.009 | 47.057 | 55.558 | 1.00 | 38.78 | A | C |
| ATOM | 1431 | CB | VAL | A | 201 | 43.789 | 47.788 | 54.891 | 1.00 | 39.70 | A | C |
| ATOM | 1432 | CG1 | VAL | A | 201 | 42.832 | 46.756 | 54.267 | 1.00 | 33.18 | A | C |
| ATOM | 1433 | CG2 | VAL | A | 201 | 43.040 | 48.629 | 55.935 | 1.00 | 33.79 | A | C |
| ATOM | 1434 | C | VAL | A | 201 | 45.536 | 45.983 | 54.613 | 1.00 | 38.48 | A | C |
| ATOM | 1435 | O | VAL | A | 201 | 45.413 | 44.786 | 54.900 | 1.00 | 35.09 | A | O |
| ATOM | 1436 | N | LYS | A | 202 | 46.138 | 46.414 | 53.504 | 1.00 | 39.99 | A | N |
| ATOM | 1437 | CA | LYS | A | 202 | 46.690 | 45.489 | 52.511 | 1.00 | 45.03 | A | C |
| ATOM | 1438 | CB | LYS | A | 202 | 47.350 | 46.259 | 51.354 | 1.00 | 47.23 | A | C |
| ATOM | 1439 | CG | LYS | A | 202 | 46.400 | 47.072 | 50.496 | 1.00 | 55.07 | A | C |
| ATOM | 1440 | CD | LYS | A | 202 | 47.145 | 47.968 | 49.500 | 1.00 | 61.48 | A | C |
| ATOM | 1441 | CE | LYS | A | 202 | 46.176 | 48.927 | 48.801 | 1.00 | 66.94 | A | C |
| ATOM | 1442 | NZ | LYS | A | 202 | 46.834 | 50.028 | 48.034 | 1.00 | 70.22 | A | N |
| ATOM | 1443 | C | LYS | A | 202 | 47.714 | 44.532 | 53.124 | 1.00 | 43.31 | A | C |
| ATOM | 1444 | O | LYS | A | 202 | 47.728 | 43.338 | 52.804 | 1.00 | 43.20 | A | O |
| ATOM | 1445 | N | ASP | A | 203 | 48.566 | 45.048 | 54.004 | 1.00 | 41.00 | A | N |
| ATOM | 1446 | CA | ASP | A | 203 | 49.579 | 44.203 | 54.629 | 1.00 | 42.52 | A | C |
| ATOM | 1447 | CB | ASP | A | 203 | 50.499 | 45.012 | 55.550 | 1.00 | 41.92 | A | C |
| ATOM | 1448 | CG | ASP | A | 203 | 51.404 | 45.968 | 54.787 | 1.00 | 47.25 | A | C |
| ATOM | 1449 | OD1 | ASP | A | 203 | 51.526 | 45.827 | 53.547 | 1.00 | 48.63 | A | O |
| ATOM | 1450 | OD2 | ASP | A | 203 | 52.007 | 46.860 | 55.431 | 1.00 | 47.22 | A | O |
| ATOM | 1451 | C | ASP | A | 203 | 48.969 | 43.058 | 55.425 | 1.00 | 41.75 | A | C |
| ATOM | 1452 | O | ASP | A | 203 | 49.321 | 41.896 | 55.195 | 1.00 | 42.78 | A | O |
| ATOM | 1453 | N | ALA | A | 204 | 48.071 | 43.374 | 56.360 | 1.00 | 39.27 | A | N |
| ATOM | 1454 | CA | ALA | A | 204 | 47.432 | 42.337 | 57.184 | 1.00 | 39.06 | A | C |
| ATOM | 1455 | CB | ALA | A | 204 | 46.504 | 42.975 | 58.202 | 1.00 | 37.36 | A | C |
| ATOM | 1456 | C | ALA | A | 204 | 46.662 | 41.353 | 56.300 | 1.00 | 39.06 | A | C |
| ATOM | 1457 | O | ALA | A | 204 | 46.596 | 40.154 | 56.576 | 1.00 | 38.46 | A | O |
| ATOM | 1458 | N | TYR | A | 205 | 46.082 | 41.863 | 55.224 | 1.00 | 40.99 | A | N |
| ATOM | 1459 | CA | TYR | A | 205 | 45.353 | 41.004 | 54.313 | 1.00 | 43.18 | A | C |
| ATOM | 1460 | CB | TYR | A | 205 | 44.673 | 41.828 | 53.225 | 1.00 | 42.13 | A | C |
| ATOM | 1461 | CG | TYR | A | 205 | 44.121 | 40.970 | 52.111 | 1.00 | 45.57 | A | C |
| ATOM | 1462 | CD1 | TYR | A | 205 | 42.916 | 40.282 | 52.266 | 1.00 | 45.90 | A | C |
| ATOM | 1463 | CE1 | TYR | A | 205 | 42.428 | 39.444 | 51.264 | 1.00 | 46.31 | A | C |
| ATOM | 1464 | CD2 | TYR | A | 205 | 44.827 | 40.804 | 50.921 | 1.00 | 43.35 | A | C |
| ATOM | 1465 | CE2 | TYR | A | 205 | 44.351 | 39.962 | 49.910 | 1.00 | 47.09 | A | C |
| ATOM | 1466 | CZ | TYR | A | 205 | 43.153 | 39.282 | 50.092 | 1.00 | 47.80 | A | C |
| ATOM | 1467 | OH | TYR | A | 205 | 42.704 | 38.413 | 49.127 | 1.00 | 46.37 | A | O |
| ATOM | 1468 | C | TYR | A | 205 | 46.325 | 40.024 | 53.663 | 1.00 | 43.34 | A | C |
| ATOM | 1469 | O | TYR | A | 205 | 46.112 | 38.810 | 53.701 | 1.00 | 44.16 | A | O |
| ATOM | 1470 | N | ASP | A | 206 | 47.391 | 40.556 | 53.063 | 1.00 | 42.55 | A | N |
| ATOM | 1471 | CA | ASP | A | 206 | 48.389 | 39.716 | 52.391 | 1.00 | 42.52 | A | C |
| ATOM | 1472 | CB | ASP | A | 206 | 49.597 | 40.552 | 51.939 | 1.00 | 43.58 | A | C |
| ATOM | 1473 | CG | ASP | A | 206 | 49.256 | 41.521 | 50.811 | 1.00 | 49.33 | A | C |
| ATOM | 1474 | OD1 | ASP | A | 206 | 48.367 | 41.209 | 49.993 | 1.00 | 52.39 | A | O |
| ATOM | 1475 | OD2 | ASP | A | 206 | 49.890 | 42.596 | 50.726 | 1.00 | 53.30 | A | O |
| ATOM | 1476 | C | ASP | A | 206 | 48.864 | 38.573 | 53.287 | 1.00 | 40.83 | A | C |
| ATOM | 1477 | O | ASP | A | 206 | 48.984 | 37.425 | 52.846 | 1.00 | 40.81 | A | O |
| ATOM | 1478 | N | MET | A | 207 | 49.120 | 38.880 | 54.551 | 1.00 | 38.05 | A | N |
| ATOM | 1479 | CA | MET | A | 207 | 49.579 | 37.865 | 55.483 | 1.00 | 41.09 | A | C |
| ATOM | 1480 | CB | MET | A | 207 | 50.018 | 38.501 | 56.791 | 1.00 | 40.14 | A | C |
| ATOM | 1481 | CG | MET | A | 207 | 50.703 | 37.530 | 57.724 | 1.00 | 47.44 | A | C |
| ATOM | 1482 | SD | MET | A | 207 | 52.318 | 36.934 | 57.042 | 1.00 | 54.26 | A | S |
| ATOM | 1483 | CE | MET | A | 207 | 52.056 | 35.165 | 57.083 | 1.00 | 52.44 | A | C |
| ATOM | 1484 | C | MET | A | 207 | 48.518 | 36.821 | 55.790 | 1.00 | 43.32 | A | C |
| ATOM | 1485 | O | MET | A | 207 | 48.822 | 35.630 | 55.870 | 1.00 | 45.30 | A | O |
| ATOM | 1486 | N | ALA | A | 208 | 47.277 | 37.266 | 55.995 | 1.00 | 42.72 | A | N |
| ATOM | 1487 | CA | ALA | A | 208 | 46.204 | 36.334 | 56.308 | 1.00 | 42.04 | A | C |
| ATOM | 1488 | CB | ALA | A | 208 | 44.904 | 37.093 | 56.695 | 1.00 | 38.76 | A | C |
| ATOM | 1489 | C | ALA | A | 208 | 45.964 | 35.458 | 55.093 | 1.00 | 40.12 | A | C |
| ATOM | 1490 | O | ALA | A | 208 | 45.743 | 34.258 | 55.223 | 1.00 | 38.22 | A | O |
| ATOM | 1491 | N | LYS | A | 209 | 46.015 | 36.076 | 53.915 | 1.00 | 40.19 | A | N |
| ATOM | 1492 | CA | LYS | A | 209 | 45.806 | 35.379 | 52.647 | 1.00 | 44.83 | A | C |
| ATOM | 1493 | CB | LYS | A | 209 | 45.980 | 36.342 | 51.474 | 1.00 | 44.87 | A | C |
| ATOM | 1494 | CG | LYS | A | 209 | 45.939 | 35.660 | 50.099 | 1.00 | 42.12 | A | C |
| ATOM | 1495 | CD | LYS | A | 209 | 46.131 | 36.684 | 48.986 | 1.00 | 42.12 | A | C |
| ATOM | 1496 | CE | LYS | A | 209 | 45.619 | 36.157 | 47.663 | 1.00 | 44.97 | A | C |
| ATOM | 1497 | NZ | LYS | A | 209 | 46.181 | 34.807 | 47.382 | 1.00 | 49.10 | A | N |
| ATOM | 1498 | C | LYS | A | 209 | 46.793 | 34.236 | 52.483 | 1.00 | 49.27 | A | C |
| ATOM | 1499 | O | LYS | A | 209 | 46.428 | 33.116 | 52.100 | 1.00 | 50.26 | A | O |
| ATOM | 1500 | N | LEU | A | 210 | 48.053 | 34.540 | 52.766 | 1.00 | 51.60 | A | N |
| ATOM | 1501 | CA | LEU | A | 210 | 49.103 | 33.555 | 52.666 | 1.00 | 55.35 | A | C |
| ATOM | 1502 | CB | LEU | A | 210 | 50.425 | 34.170 | 53.119 | 1.00 | 60.26 | A | C |
| ATOM | 1503 | CG | LEU | A | 210 | 51.713 | 33.450 | 52.724 | 1.00 | 64.09 | A | C |
| ATOM | 1504 | CD1 | LEU | A | 210 | 52.898 | 34.388 | 52.926 | 1.00 | 67.27 | A | C |
| ATOM | 1505 | CD2 | LEU | A | 210 | 51.873 | 32.192 | 53.550 | 1.00 | 66.89 | A | C |
| ATOM | 1506 | C | LEU | A | 210 | 48.746 | 32.347 | 53.523 | 1.00 | 54.22 | A | C |
| ATOM | 1507 | O | LEU | A | 210 | 48.822 | 31.209 | 53.058 | 1.00 | 55.40 | A | O |
| ATOM | 1508 | N | LEU | A | 211 | 48.351 | 32.593 | 54.768 | 1.00 | 51.60 | A | N |
| ATOM | 1509 | CA | LEU | A | 211 | 47.993 | 31.503 | 55.668 | 1.00 | 50.99 | A | C |
| ATOM | 1510 | CB | LEU | A | 211 | 47.758 | 32.031 | 57.081 | 1.00 | 48.18 | A | C |
| ATOM | 1511 | CG | LEU | A | 211 | 48.942 | 32.680 | 57.795 | 1.00 | 47.82 | A | C |
| ATOM | 1512 | CD1 | LEU | A | 211 | 48.516 | 33.120 | 59.193 | 1.00 | 40.94 | A | C |
| ATOM | 1513 | CD2 | LEU | A | 211 | 50.096 | 31.675 | 57.873 | 1.00 | 47.93 | A | C |
| ATOM | 1514 | C | LEU | A | 211 | 46.736 | 30.797 | 55.182 | 1.00 | 52.91 | A | C |
| ATOM | 1515 | O | LEU | A | 211 | 46.647 | 29.565 | 55.196 | 1.00 | 53.03 | A | O |
| ATOM | 1516 | N | CYS | A | 212 | 45.761 | 31.585 | 54.751 | 1.00 | 53.92 | A | N |
| ATOM | 1517 | CA | CYS | A | 212 | 44.514 | 31.026 | 54.267 | 1.00 | 55.97 | A | C |
| ATOM | 1518 | CB | CYS | A | 212 | 43.545 | 32.146 | 53.879 | 1.00 | 53.62 | A | C |
| ATOM | 1519 | SG | CYS | A | 212 | 41.894 | 31.550 | 53.518 | 1.00 | 53.26 | A | S |
| ATOM | 1520 | C | CYS | A | 212 | 44.775 | 30.112 | 53.069 | 1.00 | 55.91 | A | C |
| ATOM | 1521 | O | CYS | A | 212 | 44.184 | 29.039 | 52.968 | 1.00 | 53.08 | A | O |
| ATOM | 1522 | N | ASP | A | 213 | 45.665 | 30.533 | 52.173 | 1.00 | 58.04 | A | N |
| ATOM | 1523 | CA | ASP | A | 213 | 45.979 | 29.724 | 50.998 | 1.00 | 59.70 | A | C |
| ATOM | 1524 | CB | ASP | A | 213 | 46.859 | 30.504 | 50.020 | 1.00 | 57.31 | A | C |
| ATOM | 1525 | CG | ASP | A | 213 | 46.066 | 31.492 | 49.184 | 1.00 | 58.04 | A | C |
| ATOM | 1526 | OD1 | ASP | A | 213 | 44.840 | 31.295 | 49.024 | 1.00 | 57.99 | A | O |
| ATOM | 1527 | OD2 | ASP | A | 213 | 46.672 | 32.456 | 48.668 | 1.00 | 56.90 | A | O |
| ATOM | 1528 | C | ASP | A | 213 | 46.664 | 28.407 | 51.369 | 1.00 | 61.41 | A | C |
| ATOM | 1529 | O | ASP | A | 213 | 46.348 | 27.362 | 50.813 | 1.00 | 62.30 | A | O |
| ATOM | 1530 | N | LYS | A | 214 | 47.586 | 28.459 | 52.323 | 1.00 | 62.73 | A | N |
| ATOM | 1531 | CA | LYS | A | 214 | 48.316 | 27.271 | 52.752 | 1.00 | 64.81 | A | C |
| ATOM | 1532 | CB | LYS | A | 214 | 49.457 | 27.669 | 53.696 | 1.00 | 68.13 | A | C |
| ATOM | 1533 | CG | LYS | A | 214 | 50.846 | 27.217 | 53.251 | 1.00 | 73.73 | A | C |
| ATOM | 1534 | CD | LYS | A | 214 | 51.899 | 27.501 | 54.323 | 1.00 | 77.34 | A | C |
| ATOM | 1535 | CE | LYS | A | 214 | 51.957 | 26.403 | 55.397 | 1.00 | 80.42 | A | C |
| ATOM | 1536 | NZ | LYS | A | 214 | 53.162 | 25.523 | 55.250 | 1.00 | 82.55 | A | N |
| ATOM | 1537 | C | LYS | A | 214 | 47.433 | 26.248 | 53.462 | 1.00 | 64.46 | A | C |
| ATOM | 1538 | O | LYS | A | 214 | 47.689 | 25.046 | 53.404 | 1.00 | 64.85 | A | O |
| ATOM | 1539 | N | TYR | A | 215 | 46.391 | 26.714 | 54.132 | 1.00 | 63.80 | A | N |
| ATOM | 1540 | CA | TYR | A | 215 | 45.529 | 25.803 | 54.866 | 1.00 | 64.10 | A | C |
| ATOM | 1541 | CB | TYR | A | 215 | 45.132 | 26.450 | 56.198 | 1.00 | 67.94 | A | C |
| ATOM | 1542 | CG | TYR | A | 215 | 44.389 | 25.543 | 57.157 | 1.00 | 72.61 | A | C |
| ATOM | 1543 | CD1 | TYR | A | 215 | 43.046 | 25.229 | 56.949 | 1.00 | 74.65 | A | C |
| ATOM | 1544 | CE1 | TYR | A | 215 | 42.360 | 24.394 | 57.819 | 1.00 | 76.57 | A | C |
| ATOM | 1545 | CD2 | TYR | A | 215 | 45.030 | 24.995 | 58.270 | 1.00 | 75.25 | A | C |
| ATOM | 1546 | CE2 | TYR | A | 215 | 44.350 | 24.154 | 59.151 | 1.00 | 76.72 | A | C |
| ATOM | 1547 | CZ | TYR | A | 215 | 43.013 | 23.858 | 58.916 | 1.00 | 77.81 | A | C |
| ATOM | 1548 | OH | TYR | A | 215 | 42.321 | 23.023 | 59.764 | 1.00 | 76.44 | A | O |
| ATOM | 1549 | C | TYR | A | 215 | 44.296 | 25.359 | 54.078 | 1.00 | 63.21 | A | C |
| ATOM | 1550 | O | TYR | A | 215 | 43.893 | 24.204 | 54.170 | 1.00 | 63.79 | A | O |
| ATOM | 1551 | N | TYR | A | 216 | 43.708 | 26.257 | 53.295 | 1.00 | 62.49 | A | N |
| ATOM | 1552 | CA | TYR | A | 216 | 42.524 | 25.915 | 52.517 | 1.00 | 62.14 | A | C |
| ATOM | 1553 | CB | TYR | A | 216 | 41.397 | 26.923 | 52.768 | 1.00 | 61.07 | A | C |
| ATOM | 1554 | CG | TYR | A | 216 | 40.929 | 27.026 | 54.208 | 1.00 | 62.11 | A | C |
| ATOM | 1555 | CD1 | TYR | A | 216 | 41.360 | 28.069 | 55.030 | 1.00 | 59.70 | A | C |
| ATOM | 1556 | CE1 | TYR | A | 216 | 40.907 | 28.185 | 56.346 | 1.00 | 58.96 | A | C |
| ATOM | 1557 | CD2 | TYR | A | 216 | 40.033 | 26.092 | 54.743 | 1.00 | 61.26 | A | C |
| ATOM | 1558 | CE2 | TYR | A | 216 | 39.577 | 26.199 | 56.062 | 1.00 | 58.97 | A | C |
| ATOM | 1559 | CZ | TYR | A | 216 | 40.017 | 27.249 | 56.854 | 1.00 | 60.95 | A | C |
| ATOM | 1560 | OH | TYR | A | 216 | 39.564 | 27.369 | 58.153 | 1.00 | 60.89 | A | O |
| ATOM | 1561 | C | TYR | A | 216 | 42.795 | 25.856 | 51.017 | 1.00 | 64.24 | A | C |
| ATOM | 1562 | O | TYR | A | 216 | 41.995 | 25.309 | 50.258 | 1.00 | 64.38 | A | O |
| ATOM | 1563 | N | MET | A | 217 | 43.907 | 26.432 | 50.580 | 1.00 | 67.33 | A | N |
| ATOM | 1564 | CA | MET | A | 217 | 44.238 | 26.425 | 49.161 | 1.00 | 71.85 | A | C |
| ATOM | 1565 | CB | MET | A | 217 | 44.442 | 24.981 | 48.704 | 1.00 | 79.41 | A | C |
| ATOM | 1566 | CG | MET | A | 217 | 45.558 | 24.294 | 49.489 | 1.00 | 88.68 | A | C |
| ATOM | 1567 | SD | MET | A | 217 | 45.510 | 22.493 | 49.461 | 1.00 | 97.78 | A | S |
| ATOM | 1568 | CE | MET | A | 217 | 44.423 | 22.142 | 50.857 | 1.00 | 100.44 | A | C |
| ATOM | 1569 | C | MET | A | 217 | 43.151 | 27.128 | 48.347 | 1.00 | 69.70 | A | C |
| ATOM | 1570 | O | MET | A | 217 | 42.805 | 26.731 | 47.231 | 1.00 | 69.60 | A | O |
| ATOM | 1571 | N | ALA | A | 218 | 42.630 | 28.191 | 48.948 | 1.00 | 65.71 | A | N |
| ATOM | 1572 | CA | ALA | A | 218 | 41.601 | 29.041 | 48.371 | 1.00 | 60.59 | A | C |
| ATOM | 1573 | CB | ALA | A | 218 | 40.277 | 28.301 | 48.287 | 1.00 | 62.10 | A | C |
| ATOM | 1574 | C | ALA | A | 218 | 41.501 | 30.209 | 49.342 | 1.00 | 56.56 | A | C |
| ATOM | 1575 | O | ALA | A | 218 | 41.661 | 30.035 | 50.550 | 1.00 | 54.86 | A | O |
| ATOM | 1576 | N | SER | A | 219 | 41.264 | 31.403 | 48.824 | 1.00 | 52.77 | A | N |
| ATOM | 1577 | CA | SER | A | 219 | 41.169 | 32.565 | 49.682 | 1.00 | 49.04 | A | C |
| ATOM | 1578 | CB | SER | A | 219 | 42.561 | 33.089 | 50.033 | 1.00 | 50.02 | A | C |
| ATOM | 1579 | OG | SER | A | 219 | 43.098 | 33.837 | 48.952 | 1.00 | 50.21 | A | O |
| ATOM | 1580 | C | SER | A | 219 | 40.415 | 33.635 | 48.935 | 1.00 | 47.16 | A | C |
| ATOM | 1581 | O | SER | A | 219 | 40.572 | 33.781 | 47.727 | 1.00 | 47.47 | A | O |
| ATOM | 1582 | N | PRO | A | 220 | 39.577 | 34.403 | 49.647 | 1.00 | 46.86 | A | N |
| ATOM | 1583 | CD | PRO | A | 220 | 39.241 | 34.273 | 51.076 | 1.00 | 44.58 | A | C |
| ATOM | 1584 | CA | PRO | A | 220 | 38.796 | 35.473 | 49.018 | 1.00 | 44.72 | A | C |
| ATOM | 1585 | CB | PRO | A | 220 | 37.767 | 35.818 | 50.091 | 1.00 | 45.91 | A | C |
| ATOM | 1586 | CG | PRO | A | 220 | 38.511 | 35.574 | 51.348 | 1.00 | 44.93 | A | C |
| ATOM | 1587 | C | PRO | A | 220 | 39.695 | 36.642 | 48.693 | 1.00 | 42.57 | A | C |
| ATOM | 1588 | O | PRO | A | 220 | 40.770 | 36.753 | 49.264 | 1.00 | 40.87 | A | O |
| ATOM | 1589 | N | ASP | A | 221 | 39.259 | 37.505 | 47.783 | 1.00 | 41.94 | A | N |
| ATOM | 1590 | CA | ASP | A | 221 | 40.033 | 38.680 | 47.409 | 1.00 | 42.78 | A | C |
| ATOM | 1591 | CB | ASP | A | 221 | 39.735 | 39.067 | 45.957 | 1.00 | 46.02 | A | C |
| ATOM | 1592 | CG | ASP | A | 221 | 40.106 | 37.971 | 44.979 | 1.00 | 46.31 | A | C |
| ATOM | 1593 | OD1 | ASP | A | 221 | 41.173 | 37.347 | 45.164 | 1.00 | 46.33 | A | O |
| ATOM | 1594 | OD2 | ASP | A | 221 | 39.333 | 37.738 | 44.031 | 1.00 | 48.19 | A | O |
| ATOM | 1595 | C | ASP | A | 221 | 39.709 | 39.866 | 48.321 | 1.00 | 44.91 | A | C |
| ATOM | 1596 | O | ASP | A | 221 | 38.873 | 39.770 | 49.212 | 1.00 | 43.12 | A | O |
| ATOM | 1597 | N | LEU | A | 222 | 40.364 | 40.993 | 48.075 | 1.00 | 44.18 | A | N |
| ATOM | 1598 | CA | LEU | A | 222 | 40.152 | 42.184 | 48.870 | 1.00 | 43.86 | A | C |
| ATOM | 1599 | CB | LEU | A | 222 | 41.429 | 42.554 | 49.631 | 1.00 | 43.04 | A | C |
| ATOM | 1600 | CG | LEU | A | 222 | 41.384 | 43.873 | 50.417 | 1.00 | 43.85 | A | C |
| ATOM | 1601 | CD1 | LEU | A | 222 | 40.444 | 43.723 | 51.597 | 1.00 | 41.09 | A | C |
| ATOM | 1602 | CD2 | LEU | A | 222 | 42.783 | 44.255 | 50.906 | 1.00 | 44.47 | A | C |
| ATOM | 1603 | C | LEU | A | 222 | 39.730 | 43.378 | 48.038 | 1.00 | 45.26 | A | C |
| ATOM | 1604 | O | LEU | A | 222 | 40.227 | 43.604 | 46.940 | 1.00 | 46.91 | A | O |
| ATOM | 1605 | N | GLU | A | 223 | 38.795 | 44.143 | 48.573 | 1.00 | 44.38 | A | N |
| ATOM | 1606 | CA | GLU | A | 223 | 38.354 | 45.355 | 47.918 | 1.00 | 43.44 | A | C |
| ATOM | 1607 | CB | GLU | A | 223 | 36.911 | 45.225 | 47.435 | 1.00 | 42.65 | A | C |
| ATOM | 1608 | CG | GLU | A | 223 | 36.777 | 44.426 | 46.143 | 1.00 | 48.17 | A | C |
| ATOM | 1609 | CD | GLU | A | 223 | 35.327 | 44.216 | 45.729 | 1.00 | 48.82 | A | C |
| ATOM | 1610 | OE1 | GLU | A | 223 | 34.492 | 45.108 | 45.985 | 1.00 | 47.10 | A | O |
| ATOM | 1611 | OE2 | GLU | A | 223 | 35.029 | 43.161 | 45.138 | 1.00 | 52.03 | A | O |
| ATOM | 1612 | C | GLU | A | 223 | 38.460 | 46.397 | 49.011 | 1.00 | 41.66 | A | C |
| ATOM | 1613 | O | GLU | A | 223 | 38.045 | 46.155 | 50.150 | 1.00 | 42.58 | A | O |
| ATOM | 1614 | N | ILE | A | 224 | 39.053 | 47.535 | 48.691 | 1.00 | 38.05 | A | N |
| ATOM | 1615 | CA | ILE | A | 224 | 39.173 | 48.586 | 49.677 | 1.00 | 40.07 | A | C |
| ATOM | 1616 | CB | ILE | A | 224 | 40.643 | 48.890 | 50.047 | 1.00 | 39.56 | A | C |
| ATOM | 1617 | CG2 | ILE | A | 224 | 40.708 | 50.084 | 51.014 | 1.00 | 35.26 | A | C |
| ATOM | 1618 | CG1 | ILE | A | 224 | 41.285 | 47.666 | 50.705 | 1.00 | 39.17 | A | C |
| ATOM | 1619 | CD1 | ILE | A | 224 | 42.690 | 47.938 | 51.216 | 1.00 | 40.26 | A | C |
| ATOM | 1620 | C | ILE | A | 224 | 38.585 | 49.832 | 49.089 | 1.00 | 41.06 | A | C |
| ATOM | 1621 | O | ILE | A | 224 | 38.736 | 50.093 | 47.905 | 1.00 | 43.71 | A | O |
| ATOM | 1622 | N | GLN | A | 225 | 37.913 | 50.605 | 49.925 | 1.00 | 42.01 | A | N |
| ATOM | 1623 | CA | GLN | A | 225 | 37.310 | 51.845 | 49.484 | 1.00 | 43.05 | A | C |
| ATOM | 1624 | CB | GLN | A | 225 | 35.807 | 51.656 | 49.262 | 1.00 | 42.97 | A | C |
| ATOM | 1625 | CG | GLN | A | 225 | 35.157 | 52.803 | 48.505 | 1.00 | 53.39 | A | C |
| ATOM | 1626 | CD | GLN | A | 225 | 33.638 | 52.736 | 48.517 | 1.00 | 56.26 | A | C |
| ATOM | 1627 | OE1 | GLN | A | 225 | 33.043 | 51.663 | 48.363 | 1.00 | 58.43 | A | O |
| ATOM | 1628 | NE2 | GLN | A | 225 | 33.002 | 53.887 | 48.688 | 1.00 | 58.72 | A | N |
| ATOM | 1629 | C | GLN | A | 225 | 37.546 | 52.884 | 50.569 | 1.00 | 40.60 | A | C |
| ATOM | 1630 | O | GLN | A | 225 | 37.349 | 52.611 | 51.749 | 1.00 | 43.75 | A | O |
| ATOM | 1631 | N | GLU | A | 226 | 37.976 | 54.071 | 50.175 | 1.00 | 39.66 | A | N |
| ATOM | 1632 | CA | GLU | A | 226 | 38.216 | 55.129 | 51.133 | 1.00 | 41.10 | A | C |
| ATOM | 1633 | CB | GLU | A | 226 | 39.594 | 55.743 | 50.903 | 1.00 | 41.55 | A | C |
| ATOM | 1634 | CG | GLU | A | 226 | 40.721 | 54.749 | 51.043 | 1.00 | 43.93 | A | C |
| ATOM | 1635 | CD | GLU | A | 226 | 42.049 | 55.404 | 51.374 | 1.00 | 48.27 | A | C |
| ATOM | 1636 | OE1 | GLU | A | 226 | 43.006 | 54.652 | 51.669 | 1.00 | 50.51 | A | O |
| ATOM | 1637 | OE2 | GLU | A | 226 | 42.147 | 56.656 | 51.341 | 1.00 | 47.43 | A | O |
| ATOM | 1638 | C | GLU | A | 226 | 37.145 | 56.205 | 51.009 | 1.00 | 41.13 | A | C |
| ATOM | 1639 | O | GLU | A | 226 | 36.630 | 56.453 | 49.924 | 1.00 | 41.21 | A | O |
| ATOM | 1640 | N | ILE | A | 227 | 36.805 | 56.826 | 52.134 | 1.00 | 42.06 | A | N |
| ATOM | 1641 | CA | ILE | A | 227 | 35.813 | 57.886 | 52.166 | 1.00 | 41.96 | A | C |
| ATOM | 1642 | CB | ILE | A | 227 | 34.505 | 57.415 | 52.846 | 1.00 | 41.91 | A | C |
| ATOM | 1643 | CG2 | ILE | A | 227 | 33.513 | 58.562 | 52.916 | 1.00 | 42.86 | A | C |
| ATOM | 1644 | CG1 | ILE | A | 227 | 33.907 | 56.233 | 52.080 | 1.00 | 43.83 | A | C |
| ATOM | 1645 | CD1 | ILE | A | 227 | 33.454 | 56.565 | 50.667 | 1.00 | 43.79 | A | C |
| ATOM | 1646 | C | ILE | A | 227 | 36.411 | 59.019 | 52.985 | 1.00 | 42.00 | A | C |
| ATOM | 1647 | O | ILE | A | 227 | 36.222 | 59.083 | 54.200 | 1.00 | 42.49 | A | O |
| ATOM | 1648 | N | ASN | A | 228 | 37.159 | 59.895 | 52.333 | 1.00 | 42.73 | A | N |
| ATOM | 1649 | CA | ASN | A | 228 | 37.755 | 61.013 | 53.045 | 1.00 | 45.91 | A | C |
| ATOM | 1650 | CB | ASN | A | 228 | 39.050 | 61.444 | 52.372 | 1.00 | 42.90 | A | C |
| ATOM | 1651 | CG | ASN | A | 228 | 39.865 | 62.386 | 53.231 | 1.00 | 44.12 | A | C |
| ATOM | 1652 | OD1 | ASN | A | 228 | 39.322 | 63.248 | 53.941 | 1.00 | 41.39 | A | O |
| ATOM | 1653 | ND2 | ASN | A | 228 | 41.185 | 62.241 | 53.160 | 1.00 | 43.03 | A | N |
| ATOM | 1654 | C | ASN | A | 228 | 36.746 | 62.144 | 52.973 | 1.00 | 47.82 | A | C |
| ATOM | 1655 | O | ASN | A | 228 | 36.769 | 62.935 | 52.037 | 1.00 | 50.60 | A | O |
| ATOM | 1656 | N | ALA | A | 229 | 35.864 | 62.212 | 53.964 | 1.00 | 49.56 | A | N |
| ATOM | 1657 | CA | ALA | A | 229 | 34.810 | 63.216 | 54.017 | 1.00 | 51.89 | A | C |
| ATOM | 1658 | CB | ALA | A | 229 | 34.171 | 63.214 | 55.403 | 1.00 | 52.14 | A | C |
| ATOM | 1659 | C | ALA | A | 229 | 35.207 | 64.642 | 53.628 | 1.00 | 53.53 | A | C |
| ATOM | 1660 | O | ALA | A | 229 | 34.696 | 65.185 | 52.651 | 1.00 | 54.11 | A | O |
| ATOM | 1661 | N | ALA | A | 230 | 36.114 | 65.242 | 54.391 | 1.00 | 56.16 | A | N |
| ATOM | 1662 | CA | ALA | A | 230 | 36.547 | 66.614 | 54.148 | 1.00 | 56.29 | A | C |
| ATOM | 1663 | CB | ALA | A | 230 | 37.265 | 67.150 | 55.383 | 1.00 | 54.50 | A | C |
| ATOM | 1664 | C | ALA | A | 230 | 37.413 | 66.829 | 52.912 | 1.00 | 58.38 | A | C |
| ATOM | 1665 | O | ALA | A | 230 | 37.366 | 67.900 | 52.308 | 1.00 | 57.37 | A | O |
| ATOM | 1666 | N | ASN | A | 231 | 38.207 | 65.826 | 52.542 | 1.00 | 59.92 | A | N |
| ATOM | 1667 | CA | ASN | A | 231 | 39.078 | 65.931 | 51.371 | 1.00 | 61.12 | A | C |
| ATOM | 1668 | CB | ASN | A | 231 | 40.539 | 66.048 | 51.806 | 1.00 | 61.96 | A | C |
| ATOM | 1669 | CG | ASN | A | 231 | 40.803 | 67.314 | 52.593 | 1.00 | 66.50 | A | C |
| ATOM | 1670 | OD1 | ASN | A | 231 | 40.287 | 67.492 | 53.699 | 1.00 | 66.40 | A | O |
| ATOM | 1671 | ND2 | ASN | A | 231 | 41.602 | 68.210 | 52.021 | 1.00 | 68.52 | A | N |
| ATOM | 1672 | C | ASN | A | 231 | 38.891 | 64.750 | 50.418 | 1.00 | 62.40 | A | C |
| ATOM | 1673 | O | ASN | A | 231 | 39.683 | 63.810 | 50.392 | 1.00 | 59.06 | A | O |
| ATOM | 1674 | N | SER | A | 232 | 37.830 | 64.845 | 49.624 | 1.00 | 65.76 | A | N |
| ATOM | 1675 | CA | SER | A | 232 | 37.418 | 63.834 | 48.656 | 1.00 | 69.96 | A | C |
| ATOM | 1676 | CB | SER | A | 232 | 36.797 | 64.516 | 47.429 | 1.00 | 71.64 | A | C |
| ATOM | 1677 | OG | SER | A | 232 | 36.129 | 63.564 | 46.615 | 1.00 | 70.56 | A | O |
| ATOM | 1678 | C | SER | A | 232 | 38.400 | 62.759 | 48.185 | 1.00 | 71.82 | A | C |
| ATOM | 1679 | O | SER | A | 232 | 38.036 | 61.590 | 48.128 | 1.00 | 72.74 | A | O |
| ATOM | 1680 | N | LYS | A | 233 | 39.622 | 63.109 | 47.813 | 1.00 | 74.92 | A | N |
| ATOM | 1681 | CA | LYS | A | 233 | 40.506 | 62.037 | 47.372 | 1.00 | 78.22 | A | C |
| ATOM | 1682 | CB | LYS | A | 233 | 40.482 | 61.910 | 45.849 | 1.00 | 81.92 | A | C |
| ATOM | 1683 | CG | LYS | A | 233 | 39.389 | 60.966 | 45.365 | 1.00 | 85.66 | A | C |
| ATOM | 1684 | CD | LYS | A | 233 | 38.514 | 61.620 | 44.312 | 1.00 | 89.41 | A | C |
| ATOM | 1685 | CE | LYS | A | 233 | 37.283 | 60.780 | 44.017 | 1.00 | 90.98 | A | C |
| ATOM | 1686 | NZ | LYS | A | 233 | 36.306 | 61.531 | 43.174 | 1.00 | 93.45 | A | N |
| ATOM | 1687 | C | LYS | A | 233 | 41.931 | 62.047 | 47.866 | 1.00 | 77.79 | A | C |
| ATOM | 1688 | O | LYS | A | 233 | 42.797 | 61.379 | 47.302 | 1.00 | 78.39 | A | O |
| ATOM | 1689 | N | GLN | A | 234 | 42.182 | 62.807 | 48.920 | 1.00 | 76.67 | A | N |
| ATOM | 1690 | CA | GLN | A | 234 | 43.509 | 62.810 | 49.490 | 1.00 | 76.13 | A | C |
| ATOM | 1691 | CB | GLN | A | 234 | 43.678 | 63.978 | 50.473 | 1.00 | 82.07 | A | C |
| ATOM | 1692 | CG | GLN | A | 234 | 43.652 | 65.359 | 49.824 | 1.00 | 90.66 | A | C |
| ATOM | 1693 | CD | GLN | A | 234 | 44.694 | 65.508 | 48.724 | 1.00 | 95.21 | A | C |
| ATOM | 1694 | OE1 | GLN | A | 234 | 44.603 | 64.864 | 47.677 | 1.00 | 96.93 | A | O |
| ATOM | 1695 | NE2 | GLN | A | 234 | 45.692 | 66.355 | 48.961 | 1.00 | 97.75 | A | N |
| ATOM | 1696 | C | GLN | A | 234 | 43.566 | 61.469 | 50.226 | 1.00 | 71.62 | A | C |
| ATOM | 1697 | O | GLN | A | 234 | 42.616 | 61.094 | 50.921 | 1.00 | 69.15 | A | O |
| ATOM | 1698 | N | PRO | A | 235 | 44.654 | 60.704 | 50.043 | 1.00 | 67.64 | A | N |
| ATOM | 1699 | CD | PRO | A | 235 | 45.799 | 60.902 | 49.136 | 1.00 | 66.61 | A | C |
| ATOM | 1700 | CA | PRO | A | 235 | 44.745 | 59.415 | 50.738 | 1.00 | 62.61 | A | C |
| ATOM | 1701 | CB | PRO | A | 235 | 46.135 | 58.920 | 50.358 | 1.00 | 63.74 | A | C |
| ATOM | 1702 | CG | PRO | A | 235 | 46.323 | 59.487 | 48.977 | 1.00 | 65.50 | A | C |
| ATOM | 1703 | C | PRO | A | 235 | 44.580 | 59.615 | 52.251 | 1.00 | 58.27 | A | C |
| ATOM | 1704 | O | PRO | A | 235 | 45.009 | 60.627 | 52.805 | 1.00 | 56.45 | A | O |
| ATOM | 1705 | N | ILE | A | 236 | 43.951 | 58.651 | 52.914 | 1.00 | 53.17 | A | N |
| ATOM | 1706 | CA | ILE | A | 236 | 43.717 | 58.746 | 54.350 | 1.00 | 48.84 | A | C |
| ATOM | 1707 | CB | ILE | A | 236 | 42.546 | 57.845 | 54.769 | 1.00 | 47.99 | A | C |
| ATOM | 1708 | CG2 | ILE | A | 236 | 42.437 | 57.793 | 56.292 | 1.00 | 43.57 | A | C |
| ATOM | 1709 | CG1 | ILE | A | 236 | 41.258 | 58.359 | 54.123 | 1.00 | 44.40 | A | C |
| ATOM | 1710 | CD1 | ILE | A | 236 | 40.092 | 57.395 | 54.221 | 1.00 | 45.75 | A | C |
| ATOM | 1711 | C | ILE | A | 236 | 44.936 | 58.383 | 55.186 | 1.00 | 47.66 | A | C |
| ATOM | 1712 | O | ILE | A | 236 | 45.396 | 57.245 | 55.165 | 1.00 | 47.62 | A | O |
| ATOM | 1713 | N | HIS | A | 237 | 45.453 | 59.363 | 55.920 | 1.00 | 48.51 | A | N |
| ATOM | 1714 | CA | HIS | A | 237 | 46.612 | 59.167 | 56.797 | 1.00 | 49.18 | A | C |
| ATOM | 1715 | CB | HIS | A | 237 | 47.798 | 60.048 | 56.373 | 1.00 | 52.45 | A | C |
| ATOM | 1716 | CG | HIS | A | 237 | 48.452 | 59.619 | 55.098 | 1.00 | 61.42 | A | C |
| ATOM | 1717 | CD2 | HIS | A | 237 | 49.658 | 59.044 | 54.864 | 1.00 | 65.48 | A | C |
| ATOM | 1718 | ND1 | HIS | A | 237 | 47.854 | 59.759 | 53.862 | 1.00 | 65.91 | A | N |
| ATOM | 1719 | CE1 | HIS | A | 237 | 48.660 | 59.290 | 52.926 | 1.00 | 66.16 | A | C |
| ATOM | 1720 | NE2 | HIS | A | 237 | 49.763 | 58.849 | 53.508 | 1.00 | 66.13 | A | N |
| ATOM | 1721 | C | HIS | A | 237 | 46.185 | 59.565 | 58.200 | 1.00 | 46.34 | A | C |
| ATOM | 1722 | O | HIS | A | 237 | 45.226 | 60.316 | 58.377 | 1.00 | 45.46 | A | O |
| ATOM | 1723 | N | MET | A | 238 | 46.897 | 59.058 | 59.194 | 1.00 | 43.68 | A | N |
| ATOM | 1724 | CA | MET | A | 238 | 46.600 | 59.382 | 60.577 | 1.00 | 39.08 | A | C |
| ATOM | 1725 | CB | MET | A | 238 | 45.512 | 58.460 | 61.123 | 1.00 | 38.71 | A | C |
| ATOM | 1726 | CG | MET | A | 238 | 45.988 | 57.021 | 61.355 | 1.00 | 36.23 | A | C |
| ATOM | 1727 | SD | MET | A | 238 | 44.730 | 55.940 | 62.100 | 1.00 | 34.96 | A | S |
| ATOM | 1728 | CE | MET | A | 238 | 44.322 | 56.841 | 63.631 | 1.00 | 34.78 | A | C |
| ATOM | 1729 | C | MET | A | 238 | 47.876 | 59.145 | 61.361 | 1.00 | 40.31 | A | C |
| ATOM | 1730 | O | MET | A | 238 | 48.842 | 58.567 | 60.838 | 1.00 | 38.26 | A | O |
| ATOM | 1731 | N | VAL | A | 239 | 47.882 | 59.603 | 62.607 | 1.00 | 40.77 | A | N |
| ATOM | 1732 | CA | VAL | A | 239 | 49.017 | 59.384 | 63.484 | 1.00 | 41.86 | A | C |
| ATOM | 1733 | CB | VAL | A | 239 | 49.252 | 60.584 | 64.436 | 1.00 | 42.27 | A | C |
| ATOM | 1734 | CG1 | VAL | A | 239 | 50.452 | 60.308 | 65.349 | 1.00 | 40.07 | A | C |
| ATOM | 1735 | CG2 | VAL | A | 239 | 49.488 | 61.859 | 63.622 | 1.00 | 42.54 | A | C |
| ATOM | 1736 | C | VAL | A | 239 | 48.573 | 58.172 | 64.301 | 1.00 | 44.37 | A | C |
| ATOM | 1737 | O | VAL | A | 239 | 47.544 | 58.225 | 65.002 | 1.00 | 44.72 | A | O |
| ATOM | 1738 | N | TYR | A | 240 | 49.306 | 57.066 | 64.197 | 1.00 | 41.19 | A | N |
| ATOM | 1739 | CA | TYR | A | 240 | 48.920 | 55.883 | 64.962 | 1.00 | 40.26 | A | C |
| ATOM | 1740 | CB | TYR | A | 240 | 47.739 | 55.180 | 64.262 | 1.00 | 37.76 | A | C |
| ATOM | 1741 | CG | TYR | A | 240 | 46.887 | 54.338 | 65.193 | 1.00 | 37.72 | A | C |
| ATOM | 1742 | CD1 | TYR | A | 240 | 46.868 | 52.930 | 65.098 | 1.00 | 33.44 | A | C |
| ATOM | 1743 | CE1 | TYR | A | 240 | 46.151 | 52.153 | 66.027 | 1.00 | 30.13 | A | C |
| ATOM | 1744 | CD2 | TYR | A | 240 | 46.163 | 54.938 | 66.228 | 1.00 | 32.48 | A | C |
| ATOM | 1745 | CE2 | TYR | A | 240 | 45.453 | 54.171 | 67.151 | 1.00 | 33.84 | A | C |
| ATOM | 1746 | CZ | TYR | A | 240 | 45.453 | 52.785 | 67.044 | 1.00 | 30.96 | A | C |
| ATOM | 1747 | OH | TYR | A | 240 | 44.736 | 52.064 | 67.960 | 1.00 | 31.27 | A | O |
| ATOM | 1748 | C | TYR | A | 240 | 50.093 | 54.921 | 65.130 | 1.00 | 40.43 | A | C |
| ATOM | 1749 | O | TYR | A | 240 | 51.192 | 55.168 | 64.615 | 1.00 | 41.37 | A | O |
| ATOM | 1750 | N | VAL | A | 241 | 49.872 | 53.841 | 65.878 | 1.00 | 40.15 | A | N |
| ATOM | 1751 | CA | VAL | A | 241 | 50.920 | 52.824 | 66.073 | 1.00 | 38.57 | A | C |
| ATOM | 1752 | CB | VAL | A | 241 | 50.881 | 52.254 | 67.490 | 1.00 | 38.57 | A | C |
| ATOM | 1753 | CG1 | VAL | A | 241 | 52.048 | 51.264 | 67.689 | 1.00 | 36.24 | A | C |
| ATOM | 1754 | CG2 | VAL | A | 241 | 50.933 | 53.409 | 68.500 | 1.00 | 36.47 | A | C |
| ATOM | 1755 | C | VAL | A | 241 | 50.580 | 51.728 | 65.070 | 1.00 | 38.70 | A | C |
| ATOM | 1756 | O | VAL | A | 241 | 49.727 | 50.873 | 65.336 | 1.00 | 37.52 | A | O |
| ATOM | 1757 | N | PRO | A | 242 | 51.243 | 51.738 | 63.902 | 1.00 | 38.05 | A | N |
| ATOM | 1758 | CD | PRO | A | 242 | 52.331 | 52.640 | 63.485 | 1.00 | 35.78 | A | C |
| ATOM | 1759 | CA | PRO | A | 242 | 50.967 | 50.735 | 62.869 | 1.00 | 39.10 | A | C |
| ATOM | 1760 | CB | PRO | A | 242 | 52.009 | 51.042 | 61.780 | 1.00 | 37.84 | A | C |
| ATOM | 1761 | CG | PRO | A | 242 | 53.073 | 51.792 | 62.484 | 1.00 | 39.00 | A | C |
| ATOM | 1762 | C | PRO | A | 242 | 50.914 | 49.270 | 63.273 | 1.00 | 40.95 | A | C |
| ATOM | 1763 | O | PRO | A | 242 | 50.035 | 48.535 | 62.814 | 1.00 | 39.74 | A | O |
| ATOM | 1764 | N | SER | A | 243 | 51.805 | 48.829 | 64.148 | 1.00 | 41.91 | A | N |
| ATOM | 1765 | CA | SER | A | 243 | 51.753 | 47.422 | 64.524 | 1.00 | 43.52 | A | C |
| ATOM | 1766 | CB | SER | A | 243 | 53.020 | 47.003 | 65.293 | 1.00 | 42.18 | A | C |
| ATOM | 1767 | OG | SER | A | 243 | 53.028 | 47.504 | 66.606 | 1.00 | 44.50 | A | O |
| ATOM | 1768 | C | SER | A | 243 | 50.489 | 47.092 | 65.328 | 1.00 | 42.55 | A | C |
| ATOM | 1769 | O | SER | A | 243 | 50.029 | 45.940 | 65.310 | 1.00 | 40.94 | A | O |
| ATOM | 1770 | N | HIS | A | 244 | 49.924 | 48.080 | 66.026 | 1.00 | 40.58 | A | N |
| ATOM | 1771 | CA | HIS | A | 244 | 48.697 | 47.831 | 66.793 | 1.00 | 40.48 | A | C |
| ATOM | 1772 | CB | HIS | A | 244 | 48.353 | 48.994 | 67.710 | 1.00 | 41.78 | A | C |
| ATOM | 1773 | CG | HIS | A | 244 | 49.198 | 49.079 | 68.938 | 1.00 | 48.11 | A | C |
| ATOM | 1774 | CD2 | HIS | A | 244 | 50.447 | 48.625 | 69.200 | 1.00 | 49.62 | A | C |
| ATOM | 1775 | ND1 | HIS | A | 244 | 48.780 | 49.730 | 70.081 | 1.00 | 48.64 | A | N |
| ATOM | 1776 | CE1 | HIS | A | 244 | 49.732 | 49.672 | 70.994 | 1.00 | 47.11 | A | C |
| ATOM | 1777 | NE2 | HIS | A | 244 | 50.755 | 49.007 | 70.484 | 1.00 | 50.54 | A | N |
| ATOM | 1778 | C | HIS | A | 244 | 47.543 | 47.647 | 65.820 | 1.00 | 41.19 | A | C |
| ATOM | 1779 | O | HIS | A | 244 | 46.725 | 46.728 | 65.963 | 1.00 | 38.24 | A | O |
| ATOM | 1780 | N | LEU | A | 245 | 47.497 | 48.528 | 64.822 | 1.00 | 39.92 | A | N |
| ATOM | 1781 | CA | LEU | A | 245 | 46.458 | 48.495 | 63.808 | 1.00 | 40.43 | A | C |
| ATOM | 1782 | CB | LEU | A | 245 | 46.590 | 49.704 | 62.875 | 1.00 | 36.64 | A | C |
| ATOM | 1783 | CG | LEU | A | 245 | 45.557 | 49.718 | 61.746 | 1.00 | 38.78 | A | C |
| ATOM | 1784 | CD1 | LEU | A | 245 | 44.134 | 49.738 | 62.357 | 1.00 | 35.38 | A | C |
| ATOM | 1785 | CD2 | LEU | A | 245 | 45.812 | 50.910 | 60.812 | 1.00 | 36.73 | A | C |
| ATOM | 1786 | C | LEU | A | 245 | 46.554 | 47.193 | 63.010 | 1.00 | 41.77 | A | C |
| ATOM | 1787 | O | LEU | A | 245 | 45.530 | 46.571 | 62.701 | 1.00 | 39.48 | A | O |
| ATOM | 1788 | N | TYR | A | 246 | 47.788 | 46.793 | 62.687 | 1.00 | 42.16 | A | N |
| ATOM | 1789 | CA | TYR | A | 246 | 48.045 | 45.555 | 61.949 | 1.00 | 43.07 | A | C |
| ATOM | 1790 | CB | TYR | A | 246 | 49.553 | 45.343 | 61.755 | 1.00 | 43.40 | A | C |
| ATOM | 1791 | CG | TYR | A | 246 | 49.914 | 44.048 | 61.035 | 1.00 | 45.42 | A | C |
| ATOM | 1792 | CD1 | TYR | A | 246 | 50.092 | 44.013 | 59.645 | 1.00 | 45.30 | A | C |
| ATOM | 1793 | CE1 | TYR | A | 246 | 50.402 | 42.812 | 58.984 | 1.00 | 44.56 | A | C |
| ATOM | 1794 | CD2 | TYR | A | 246 | 50.054 | 42.858 | 61.745 | 1.00 | 43.37 | A | C |
| ATOM | 1795 | CE2 | TYR | A | 246 | 50.356 | 41.660 | 61.104 | 1.00 | 42.44 | A | C |
| ATOM | 1796 | CZ | TYR | A | 246 | 50.527 | 41.637 | 59.732 | 1.00 | 46.10 | A | C |
| ATOM | 1797 | OH | TYR | A | 246 | 50.778 | 40.418 | 59.132 | 1.00 | 43.95 | A | O |
| ATOM | 1798 | C | TYR | A | 246 | 47.473 | 44.372 | 62.731 | 1.00 | 43.38 | A | C |
| ATOM | 1799 | O | TYR | A | 246 | 46.729 | 43.565 | 62.186 | 1.00 | 40.87 | A | O |
| ATOM | 1800 | N | HIS | A | 247 | 47.812 | 44.280 | 64.015 | 1.00 | 43.56 | A | N |
| ATOM | 1801 | CA | HIS | A | 247 | 47.322 | 43.179 | 64.849 | 1.00 | 45.41 | A | C |
| ATOM | 1802 | CB | HIS | A | 247 | 47.777 | 43.351 | 66.305 | 1.00 | 48.80 | A | C |
| ATOM | 1803 | CG | HIS | A | 247 | 47.204 | 42.334 | 67.245 | 1.00 | 57.37 | A | C |
| ATOM | 1804 | CD2 | HIS | A | 247 | 45.963 | 42.210 | 67.781 | 1.00 | 58.41 | A | C |
| ATOM | 1805 | ND1 | HIS | A | 247 | 47.927 | 41.255 | 67.716 | 1.00 | 61.64 | A | N |
| ATOM | 1806 | CE1 | HIS | A | 247 | 47.159 | 40.515 | 68.497 | 1.00 | 59.43 | A | C |
| ATOM | 1807 | NE2 | HIS | A | 247 | 45.962 | 41.073 | 68.553 | 1.00 | 59.86 | A | N |
| ATOM | 1808 | C | HIS | A | 247 | 45.797 | 43.078 | 64.798 | 1.00 | 42.66 | A | C |
| ATOM | 1809 | O | HIS | A | 247 | 45.242 | 41.975 | 64.663 | 1.00 | 42.64 | A | O |
| ATOM | 1810 | N | MET | A | 248 | 45.116 | 44.216 | 64.919 | 1.00 | 38.75 | A | N |
| ATOM | 1811 | CA | MET | A | 248 | 43.658 | 44.202 | 64.882 | 1.00 | 38.33 | A | C |
| ATOM | 1812 | CB | MET | A | 248 | 43.090 | 45.587 | 65.199 | 1.00 | 33.73 | A | C |
| ATOM | 1813 | CG | MET | A | 248 | 43.399 | 46.035 | 66.615 | 1.00 | 36.56 | A | C |
| ATOM | 1814 | SD | MET | A | 248 | 42.636 | 47.628 | 67.075 | 1.00 | 35.47 | A | S |
| ATOM | 1815 | CE | MET | A | 248 | 43.564 | 48.740 | 66.120 | 1.00 | 24.29 | A | C |
| ATOM | 1816 | C | MET | A | 248 | 43.158 | 43.719 | 63.522 | 1.00 | 34.80 | A | C |
| ATOM | 1817 | O | MET | A | 248 | 42.371 | 42.783 | 63.463 | 1.00 | 30.34 | A | O |
| ATOM | 1818 | N | LEU | A | 249 | 43.626 | 44.336 | 62.440 | 1.00 | 33.73 | A | N |
| ATOM | 1819 | CA | LEU | A | 249 | 43.209 | 43.950 | 61.086 | 1.00 | 37.19 | A | C |
| ATOM | 1820 | CB | LEU | A | 249 | 43.857 | 44.879 | 60.050 | 1.00 | 32.82 | A | C |
| ATOM | 1821 | CG | LEU | A | 249 | 43.247 | 46.285 | 59.992 | 1.00 | 34.06 | A | C |
| ATOM | 1822 | CD1 | LEU | A | 249 | 44.156 | 47.194 | 59.172 | 1.00 | 32.91 | A | C |
| ATOM | 1823 | CD2 | LEU | A | 249 | 41.826 | 46.221 | 59.380 | 1.00 | 30.37 | A | C |
| ATOM | 1824 | C | LEU | A | 249 | 43.527 | 42.498 | 60.742 | 1.00 | 38.20 | A | C |
| ATOM | 1825 | O | LEU | A | 249 | 42.723 | 41.799 | 60.121 | 1.00 | 41.02 | A | O |
| ATOM | 1826 | N | PHE | A | 250 | 44.701 | 42.032 | 61.142 | 1.00 | 38.53 | A | N |
| ATOM | 1827 | CA | PHE | A | 250 | 45.081 | 40.660 | 60.856 | 1.00 | 38.53 | A | C |
| ATOM | 1828 | CB | PHE | A | 250 | 46.514 | 40.388 | 61.336 | 1.00 | 40.25 | A | C |
| ATOM | 1829 | CG | PHE | A | 250 | 46.996 | 38.984 | 61.054 | 1.00 | 39.44 | A | C |
| ATOM | 1830 | CD1 | PHE | A | 250 | 47.133 | 38.527 | 59.745 | 1.00 | 39.56 | A | C |
| ATOM | 1831 | CD2 | PHE | A | 250 | 47.324 | 38.124 | 62.097 | 1.00 | 43.52 | A | C |
| ATOM | 1832 | CE1 | PHE | A | 250 | 47.598 | 37.237 | 59.477 | 1.00 | 42.41 | A | C |
| ATOM | 1833 | CE2 | PHE | A | 250 | 47.799 | 36.817 | 61.836 | 1.00 | 44.75 | A | C |
| ATOM | 1834 | CZ | PHE | A | 250 | 47.931 | 36.380 | 60.520 | 1.00 | 40.47 | A | C |
| ATOM | 1835 | C | PHE | A | 250 | 44.130 | 39.670 | 61.523 | 1.00 | 37.07 | A | C |
| ATOM | 1836 | O | PHE | A | 250 | 43.772 | 38.646 | 60.925 | 1.00 | 36.99 | A | O |
| ATOM | 1837 | N | GLU | A | 251 | 43.743 | 39.955 | 62.765 | 1.00 | 37.43 | A | N |
| ATOM | 1838 | CA | GLU | A | 251 | 42.836 | 39.069 | 63.493 | 1.00 | 39.94 | A | C |
| ATOM | 1839 | CB | GLU | A | 251 | 42.603 | 39.564 | 64.912 | 1.00 | 40.34 | A | C |
| ATOM | 1840 | CG | GLU | A | 251 | 41.789 | 38.587 | 65.740 | 1.00 | 53.45 | A | C |
| ATOM | 1841 | CD | GLU | A | 251 | 42.388 | 37.178 | 65.710 | 1.00 | 61.46 | A | C |
| ATOM | 1842 | OE1 | GLU | A | 251 | 43.608 | 37.040 | 65.983 | 1.00 | 61.50 | A | O |
| ATOM | 1843 | OE2 | GLU | A | 251 | 41.643 | 36.211 | 65.410 | 1.00 | 63.68 | A | O |
| ATOM | 1844 | C | GLU | A | 251 | 41.499 | 39.008 | 62.773 | 1.00 | 39.98 | A | C |
| ATOM | 1845 | O | GLU | A | 251 | 40.949 | 37.924 | 62.549 | 1.00 | 40.35 | A | O |
| ATOM | 1846 | N | LEU | A | 252 | 40.982 | 40.180 | 62.411 | 1.00 | 36.59 | A | N |
| ATOM | 1847 | CA | LEU | A | 252 | 39.707 | 40.258 | 61.727 | 1.00 | 37.26 | A | C |
| ATOM | 1848 | CB | LEU | A | 252 | 39.268 | 41.731 | 61.596 | 1.00 | 32.23 | A | C |
| ATOM | 1849 | CG | LEU | A | 252 | 39.011 | 42.428 | 62.952 | 1.00 | 33.53 | A | C |
| ATOM | 1850 | CD1 | LEU | A | 252 | 38.643 | 43.885 | 62.722 | 1.00 | 26.13 | A | C |
| ATOM | 1851 | CD2 | LEU | A | 252 | 37.886 | 41.711 | 63.728 | 1.00 | 29.05 | A | C |
| ATOM | 1852 | C | LEU | A | 252 | 39.782 | 39.570 | 60.362 | 1.00 | 35.30 | A | C |
| ATOM | 1853 | O | LEU | A | 252 | 38.872 | 38.809 | 60.009 | 1.00 | 35.51 | A | O |
| ATOM | 1854 | N | PHE | A | 253 | 40.853 | 39.802 | 59.601 | 1.00 | 31.86 | A | N |
| ATOM | 1855 | CA | PHE | A | 253 | 40.972 | 39.149 | 58.295 | 1.00 | 36.73 | A | C |
| ATOM | 1856 | CB | PHE | A | 253 | 42.236 | 39.606 | 57.563 | 1.00 | 34.58 | A | C |
| ATOM | 1857 | CG | PHE | A | 253 | 42.076 | 40.888 | 56.776 | 1.00 | 31.56 | A | C |
| ATOM | 1858 | CD1 | PHE | A | 253 | 41.103 | 41.004 | 55.788 | 1.00 | 26.91 | A | C |
| ATOM | 1859 | CD2 | PHE | A | 253 | 42.949 | 41.953 | 56.983 | 1.00 | 28.65 | A | C |
| ATOM | 1860 | CE1 | PHE | A | 253 | 41.009 | 42.174 | 55.001 | 1.00 | 28.87 | A | C |
| ATOM | 1861 | CE2 | PHE | A | 253 | 42.864 | 43.125 | 56.207 | 1.00 | 27.02 | A | C |
| ATOM | 1862 | CZ | PHE | A | 253 | 41.894 | 43.234 | 55.216 | 1.00 | 25.86 | A | C |
| ATOM | 1863 | C | PHE | A | 253 | 40.998 | 37.615 | 58.434 | 1.00 | 39.81 | A | C |
| ATOM | 1864 | O | PHE | A | 253 | 40.386 | 36.902 | 57.637 | 1.00 | 42.49 | A | O |
| ATOM | 1865 | N | LYS | A | 254 | 41.706 | 37.106 | 59.440 | 1.00 | 40.78 | A | N |
| ATOM | 1866 | CA | LYS | A | 254 | 41.769 | 35.657 | 59.661 | 1.00 | 42.83 | A | C |
| ATOM | 1867 | CB | LYS | A | 254 | 42.646 | 35.327 | 60.867 | 1.00 | 43.51 | A | C |
| ATOM | 1868 | CG | LYS | A | 254 | 44.118 | 35.216 | 60.526 | 1.00 | 50.83 | A | C |
| ATOM | 1869 | CD | LYS | A | 254 | 45.020 | 35.507 | 61.721 | 1.00 | 54.00 | A | C |
| ATOM | 1870 | CE | LYS | A | 254 | 44.859 | 34.515 | 62.855 | 1.00 | 56.78 | A | C |
| ATOM | 1871 | NZ | LYS | A | 254 | 45.774 | 34.848 | 63.988 | 1.00 | 59.33 | A | N |
| ATOM | 1872 | C | LYS | A | 254 | 40.393 | 35.055 | 59.878 | 1.00 | 41.28 | A | C |
| ATOM | 1873 | O | LYS | A | 254 | 40.090 | 33.987 | 59.346 | 1.00 | 39.24 | A | O |
| ATOM | 1874 | N | ASN | A | 255 | 39.561 | 35.730 | 60.669 | 1.00 | 39.33 | A | N |
| ATOM | 1875 | CA | ASN | A | 255 | 38.218 | 35.226 | 60.941 | 1.00 | 39.14 | A | C |
| ATOM | 1876 | CB | ASN | A | 255 | 37.591 | 35.955 | 62.121 | 1.00 | 39.57 | A | C |
| ATOM | 1877 | CG | ASN | A | 255 | 38.293 | 35.641 | 63.414 | 1.00 | 45.10 | A | C |
| ATOM | 1878 | OD1 | ASN | A | 255 | 38.741 | 34.511 | 63.617 | 1.00 | 47.72 | A | O |
| ATOM | 1879 | ND2 | ASN | A | 255 | 38.384 | 36.629 | 64.308 | 1.00 | 42.33 | A | N |
| ATOM | 1880 | C | ASN | A | 255 | 37.302 | 35.337 | 59.743 | 1.00 | 36.63 | A | C |
| ATOM | 1881 | O | ASN | A | 255 | 36.535 | 34.415 | 59.459 | 1.00 | 37.34 | A | O |
| ATOM | 1882 | N | ALA | A | 256 | 37.374 | 36.464 | 59.049 | 1.00 | 33.74 | A | N |
| ATOM | 1883 | CA | ALA | A | 256 | 36.545 | 36.679 | 57.876 | 1.00 | 34.48 | A | C |
| ATOM | 1884 | CB | ALA | A | 256 | 36.710 | 38.121 | 57.378 | 1.00 | 27.97 | A | C |
| ATOM | 1885 | C | ALA | A | 256 | 36.938 | 35.687 | 56.769 | 1.00 | 36.12 | A | C |
| ATOM | 1886 | O | ALA | A | 256 | 36.097 | 35.259 | 55.972 | 1.00 | 33.35 | A | O |
| ATOM | 1887 | N | MET | A | 257 | 38.215 | 35.319 | 56.711 | 1.00 | 36.79 | A | N |
| ATOM | 1888 | CA | MET | A | 257 | 38.642 | 34.388 | 55.671 | 1.00 | 40.88 | A | C |
| ATOM | 1889 | CB | MET | A | 257 | 40.169 | 34.402 | 55.510 | 1.00 | 39.59 | A | C |
| ATOM | 1890 | CG | MET | A | 257 | 40.661 | 35.609 | 54.724 | 1.00 | 41.89 | A | C |
| ATOM | 1891 | SD | MET | A | 257 | 42.452 | 35.755 | 54.628 | 1.00 | 45.89 | A | S |
| ATOM | 1892 | CE | MET | A | 257 | 42.589 | 37.242 | 53.592 | 1.00 | 37.73 | A | C |
| ATOM | 1893 | C | MET | A | 257 | 38.155 | 32.995 | 56.004 | 1.00 | 39.41 | A | C |
| ATOM | 1894 | O | MET | A | 257 | 37.532 | 32.337 | 55.188 | 1.00 | 40.08 | A | O |
| ATOM | 1895 | N | ARG | A | 258 | 38.417 | 32.562 | 57.224 | 1.00 | 39.69 | A | N |
| ATOM | 1896 | CA | ARG | A | 258 | 38.011 | 31.246 | 57.667 | 1.00 | 41.86 | A | C |
| ATOM | 1897 | CB | ARG | A | 258 | 38.332 | 31.099 | 59.153 | 1.00 | 41.36 | A | C |
| ATOM | 1898 | CG | ARG | A | 258 | 37.680 | 29.916 | 59.846 | 1.00 | 50.16 | A | C |
| ATOM | 1899 | CD | ARG | A | 258 | 38.225 | 29.816 | 61.266 | 1.00 | 58.76 | A | C |
| ATOM | 1900 | NE | ARG | A | 258 | 37.318 | 29.129 | 62.178 | 1.00 | 68.29 | A | N |
| ATOM | 1901 | CZ | ARG | A | 258 | 37.053 | 27.825 | 62.143 | 1.00 | 74.58 | A | C |
| ATOM | 1902 | NH1 | ARG | A | 258 | 37.629 | 27.044 | 61.233 | 1.00 | 76.52 | A | N |
| ATOM | 1903 | NH2 | ARG | A | 258 | 36.210 | 27.296 | 63.024 | 1.00 | 76.36 | A | N |
| ATOM | 1904 | C | ARG | A | 258 | 36.523 | 31.029 | 57.421 | 1.00 | 42.23 | A | C |
| ATOM | 1905 | O | ARG | A | 258 | 36.116 | 30.000 | 56.878 | 1.00 | 41.68 | A | O |
| ATOM | 1906 | N | ALA | A | 259 | 35.714 | 32.006 | 57.816 | 1.00 | 39.23 | A | N |
| ATOM | 1907 | CA | ALA | A | 259 | 34.268 | 31.899 | 57.663 | 1.00 | 39.90 | A | C |
| ATOM | 1908 | CB | ALA | A | 259 | 33.562 | 33.012 | 58.484 | 1.00 | 33.64 | A | C |
| ATOM | 1909 | C | ALA | A | 259 | 33.803 | 31.940 | 56.212 | 1.00 | 36.78 | A | C |
| ATOM | 1910 | O | ALA | A | 259 | 32.879 | 31.214 | 55.840 | 1.00 | 38.63 | A | O |
| ATOM | 1911 | N | THR | A | 260 | 34.410 | 32.797 | 55.399 | 1.00 | 35.71 | A | N |
| ATOM | 1912 | CA | THR | A | 260 | 34.010 | 32.891 | 53.998 | 1.00 | 39.50 | A | C |
| ATOM | 1913 | CB | THR | A | 260 | 34.777 | 34.007 | 53.259 | 1.00 | 37.67 | A | C |
| ATOM | 1914 | OG1 | THR | A | 260 | 34.525 | 35.265 | 53.894 | 1.00 | 35.27 | A | O |
| ATOM | 1915 | CG2 | THR | A | 260 | 34.324 | 34.084 | 51.783 | 1.00 | 36.95 | A | C |
| ATOM | 1916 | C | THR | A | 260 | 34.288 | 31.570 | 53.285 | 1.00 | 41.44 | A | C |
| ATOM | 1917 | O | THR | A | 260 | 33.438 | 31.039 | 52.565 | 1.00 | 41.95 | A | O |
| ATOM | 1918 | N | VAL | A | 261 | 35.488 | 31.044 | 53.503 | 1.00 | 43.54 | A | N |
| ATOM | 1919 | CA | VAL | A | 261 | 35.922 | 29.795 | 52.892 | 1.00 | 43.79 | A | C |
| ATOM | 1920 | CB | VAL | A | 261 | 37.404 | 29.493 | 53.261 | 1.00 | 42.89 | A | C |
| ATOM | 1921 | CG1 | VAL | A | 261 | 37.769 | 28.063 | 52.881 | 1.00 | 41.91 | A | C |
| ATOM | 1922 | CG2 | VAL | A | 261 | 38.318 | 30.475 | 52.553 | 1.00 | 40.68 | A | C |
| ATOM | 1923 | C | VAL | A | 261 | 35.064 | 28.610 | 53.313 | 1.00 | 44.85 | A | C |
| ATOM | 1924 | O | VAL | A | 261 | 34.540 | 27.880 | 52.471 | 1.00 | 45.57 | A | O |
| ATOM | 1925 | N | GLU | A | 262 | 34.925 | 28.419 | 54.618 | 1.00 | 44.28 | A | N |
| ATOM | 1926 | CA | GLU | A | 262 | 34.155 | 27.301 | 55.142 | 1.00 | 46.54 | A | C |
| ATOM | 1927 | CB | GLU | A | 262 | 34.360 | 27.193 | 56.653 | 1.00 | 47.54 | A | C |
| ATOM | 1928 | CG | GLU | A | 262 | 35.810 | 26.947 | 57.035 | 1.00 | 46.70 | A | C |
| ATOM | 1929 | CD | GLU | A | 262 | 35.993 | 26.642 | 58.508 | 1.00 | 51.02 | A | C |
| ATOM | 1930 | OE1 | GLU | A | 262 | 37.138 | 26.338 | 58.921 | 1.00 | 52.71 | A | O |
| ATOM | 1931 | OE2 | GLU | A | 262 | 34.999 | 26.703 | 59.261 | 1.00 | 51.77 | A | O |
| ATOM | 1932 | C | GLU | A | 262 | 32.668 | 27.331 | 54.823 | 1.00 | 49.12 | A | C |
| ATOM | 1933 | O | GLU | A | 262 | 32.054 | 26.281 | 54.627 | 1.00 | 52.71 | A | O |
| ATOM | 1934 | N | SER | A | 263 | 32.083 | 28.520 | 54.766 | 1.00 | 47.77 | A | N |
| ATOM | 1935 | CA | SER | A | 263 | 30.662 | 28.633 | 54.470 | 1.00 | 48.49 | A | C |
| ATOM | 1936 | CB | SER | A | 263 | 30.139 | 29.996 | 54.934 | 1.00 | 48.88 | A | C |
| ATOM | 1937 | OG | SER | A | 263 | 30.694 | 31.055 | 54.165 | 1.00 | 49.64 | A | O |
| ATOM | 1938 | C | SER | A | 263 | 30.392 | 28.462 | 52.974 | 1.00 | 48.83 | A | C |
| ATOM | 1939 | O | SER | A | 263 | 29.248 | 28.312 | 52.555 | 1.00 | 49.32 | A | O |
| ATOM | 1940 | N | HIS | A | 264 | 31.439 | 28.504 | 52.159 | 1.00 | 49.04 | A | N |
| ATOM | 1941 | CA | HIS | A | 264 | 31.256 | 28.345 | 50.715 | 1.00 | 49.81 | A | C |
| ATOM | 1942 | CB | HIS | A | 264 | 31.714 | 29.593 | 49.965 | 1.00 | 46.64 | A | C |
| ATOM | 1943 | CG | HIS | A | 264 | 30.840 | 30.782 | 50.189 | 1.00 | 48.72 | A | C |
| ATOM | 1944 | CD2 | HIS | A | 264 | 30.021 | 31.454 | 49.347 | 1.00 | 46.11 | A | C |
| ATOM | 1945 | ND1 | HIS | A | 264 | 30.734 | 31.409 | 51.415 | 1.00 | 46.78 | A | N |
| ATOM | 1946 | CE1 | HIS | A | 264 | 29.887 | 32.418 | 51.314 | 1.00 | 46.54 | A | C |
| ATOM | 1947 | NE2 | HIS | A | 264 | 29.440 | 32.466 | 50.071 | 1.00 | 45.76 | A | N |
| ATOM | 1948 | C | HIS | A | 264 | 31.978 | 27.145 | 50.131 | 1.00 | 51.09 | A | C |
| ATOM | 1949 | O | HIS | A | 264 | 32.330 | 27.154 | 48.958 | 1.00 | 49.62 | A | O |
| ATOM | 1950 | N | GLU | A | 265 | 32.203 | 26.116 | 50.937 | 1.00 | 55.48 | A | N |
| ATOM | 1951 | CA | GLU | A | 265 | 32.889 | 24.937 | 50.439 | 1.00 | 63.07 | A | C |
| ATOM | 1952 | CB | GLU | A | 265 | 33.164 | 23.970 | 51.582 | 1.00 | 65.74 | A | C |
| ATOM | 1953 | CG | GLU | A | 265 | 34.092 | 24.543 | 52.621 | 1.00 | 73.55 | A | C |
| ATOM | 1954 | CD | GLU | A | 265 | 34.602 | 23.496 | 53.584 | 1.00 | 77.38 | A | C |
| ATOM | 1955 | OE1 | GLU | A | 265 | 33.761 | 22.778 | 54.166 | 1.00 | 79.22 | A | O |
| ATOM | 1956 | OE2 | GLU | A | 265 | 35.841 | 23.399 | 53.758 | 1.00 | 79.32 | A | O |
| ATOM | 1957 | C | GLU | A | 265 | 32.077 | 24.237 | 49.351 | 1.00 | 64.93 | A | C |
| ATOM | 1958 | O | GLU | A | 265 | 32.226 | 23.036 | 49.128 | 1.00 | 63.89 | A | O |
| ATOM | 1959 | N | SER | A | 266 | 31.225 | 25.002 | 48.675 | 1.00 | 67.01 | A | N |
| ATOM | 1960 | CA | SER | A | 266 | 30.393 | 24.470 | 47.616 | 1.00 | 68.64 | A | C |
| ATOM | 1961 | CB | SER | A | 266 | 29.226 | 23.695 | 48.221 | 1.00 | 67.52 | A | C |
| ATOM | 1962 | OG | SER | A | 266 | 29.700 | 22.720 | 49.135 | 1.00 | 67.96 | A | O |
| ATOM | 1963 | C | SER | A | 266 | 29.862 | 25.568 | 46.712 | 1.00 | 71.22 | A | C |
| ATOM | 1964 | O | SER | A | 266 | 28.656 | 25.767 | 46.639 | 1.00 | 73.71 | A | O |
| ATOM | 1965 | N | SER | A | 267 | 30.752 | 26.283 | 46.027 | 1.00 | 73.77 | A | N |
| ATOM | 1966 | CA | SER | A | 267 | 30.316 | 27.342 | 45.115 | 1.00 | 76.52 | A | C |
| ATOM | 1967 | CB | SER | A | 267 | 29.341 | 28.287 | 45.832 | 1.00 | 77.11 | A | C |
| ATOM | 1968 | OG | SER | A | 267 | 29.681 | 28.444 | 47.196 | 1.00 | 76.08 | A | O |
| ATOM | 1969 | C | SER | A | 267 | 31.447 | 28.145 | 44.468 | 1.00 | 78.44 | A | C |
| ATOM | 1970 | O | SER | A | 267 | 32.256 | 27.597 | 43.722 | 1.00 | 79.61 | A | O |
| ATOM | 1971 | N | LEU | A | 268 | 31.486 | 29.448 | 44.739 | 1.00 | 80.81 | A | N |
| ATOM | 1972 | CA | LEU | A | 268 | 32.516 | 30.316 | 44.180 | 1.00 | 80.78 | A | C |
| ATOM | 1973 | CB | LEU | A | 268 | 31.872 | 31.463 | 43.386 | 1.00 | 84.28 | A | C |
| ATOM | 1974 | CG | LEU | A | 268 | 31.055 | 32.572 | 44.051 | 1.00 | 87.73 | A | C |
| ATOM | 1975 | CD1 | LEU | A | 268 | 30.369 | 33.371 | 42.949 | 1.00 | 89.84 | A | C |
| ATOM | 1976 | CD2 | LEU | A | 268 | 30.026 | 32.001 | 45.017 | 1.00 | 89.73 | A | C |
| ATOM | 1977 | C | LEU | A | 268 | 33.439 | 30.849 | 45.282 | 1.00 | 78.93 | A | C |
| ATOM | 1978 | O | LEU | A | 268 | 34.441 | 30.195 | 45.584 | 1.00 | 81.15 | A | O |
| ATOM | 1979 | N | ILE | A | 269 | 33.110 | 32.012 | 45.865 | 1.00 | 71.70 | A | N |
| ATOM | 1980 | CA | ILE | A | 269 | 33.890 | 32.641 | 46.959 | 1.00 | 64.88 | A | C |
| ATOM | 1981 | CB | ILE | A | 269 | 35.355 | 32.136 | 46.973 | 1.00 | 63.49 | A | C |
| ATOM | 1982 | CG2 | ILE | A | 269 | 36.085 | 32.582 | 45.700 | 1.00 | 67.54 | A | C |
| ATOM | 1983 | CG1 | ILE | A | 269 | 36.062 | 32.611 | 48.238 | 1.00 | 62.59 | A | C |
| ATOM | 1984 | CD1 | ILE | A | 269 | 37.455 | 32.033 | 48.408 | 1.00 | 57.89 | A | C |
| ATOM | 1985 | C | ILE | A | 269 | 33.893 | 34.190 | 46.955 | 1.00 | 58.83 | A | C |
| ATOM | 1986 | O | ILE | A | 269 | 34.733 | 34.831 | 46.325 | 1.00 | 57.71 | A | O |
| ATOM | 1987 | N | PRO | A | 271 | 32.954 | 34.810 | 47.686 | 1.00 | 53.24 | A | N |
| ATOM | 1988 | CD | PRO | A | 271 | 31.945 | 34.199 | 48.566 | 1.00 | 50.40 | A | C |
| ATOM | 1989 | CA | PRO | A | 271 | 32.879 | 36.278 | 47.731 | 1.00 | 49.27 | A | C |
| ATOM | 1990 | CB | PRO | A | 271 | 31.573 | 36.533 | 48.479 | 1.00 | 48.77 | A | C |
| ATOM | 1991 | CG | PRO | A | 271 | 31.502 | 35.374 | 49.410 | 1.00 | 51.48 | A | C |
| ATOM | 1992 | C | PRO | A | 271 | 34.075 | 36.981 | 48.377 | 1.00 | 45.60 | A | C |
| ATOM | 1993 | O | PRO | A | 271 | 34.746 | 36.430 | 49.258 | 1.00 | 45.16 | A | O |
| ATOM | 1994 | N | PRO | A | 272 | 34.358 | 38.215 | 47.940 | 1.00 | 41.19 | A | N |
| ATOM | 1995 | CD | PRO | A | 272 | 33.764 | 38.949 | 46.806 | 1.00 | 39.33 | A | C |
| ATOM | 1996 | CA | PRO | A | 272 | 35.490 | 38.945 | 48.515 | 1.00 | 41.11 | A | C |
| ATOM | 1997 | CB | PRO | A | 272 | 35.787 | 40.010 | 47.463 | 1.00 | 38.67 | A | C |
| ATOM | 1998 | CG | PRO | A | 272 | 34.407 | 40.328 | 46.922 | 1.00 | 37.30 | A | C |
| ATOM | 1999 | C | PRO | A | 272 | 35.193 | 39.577 | 49.869 | 1.00 | 41.24 | A | C |
| ATOM | 2000 | O | PRO | A | 272 | 34.037 | 39.717 | 50.269 | 1.00 | 40.61 | A | O |
| ATOM | 2001 | N | ILE | A | 273 | 36.259 | 39.943 | 50.569 | 1.00 | 38.85 | A | N |
| ATOM | 2002 | CA | ILE | A | 273 | 36.139 | 40.624 | 51.844 | 1.00 | 37.76 | A | C |
| ATOM | 2003 | CB | ILE | A | 273 | 37.261 | 40.199 | 52.793 | 1.00 | 31.69 | A | C |
| ATOM | 2004 | CG2 | ILE | A | 273 | 37.321 | 41.124 | 53.982 | 1.00 | 35.17 | A | C |
| ATOM | 2005 | CG1 | ILE | A | 273 | 37.002 | 38.767 | 53.253 | 1.00 | 31.18 | A | C |
| ATOM | 2006 | CD1 | ILE | A | 273 | 38.116 | 38.167 | 54.071 | 1.00 | 31.68 | A | C |
| ATOM | 2007 | C | ILE | A | 273 | 36.270 | 42.092 | 51.464 | 1.00 | 38.23 | A | C |
| ATOM | 2008 | O | ILE | A | 273 | 37.210 | 42.460 | 50.766 | 1.00 | 39.19 | A | O |
| ATOM | 2009 | N | LYS | A | 274 | 35.302 | 42.914 | 51.868 | 1.00 | 36.37 | A | N |
| ATOM | 2010 | CA | LYS | A | 274 | 35.323 | 44.339 | 51.541 | 1.00 | 37.42 | A | C |
| ATOM | 2011 | CB | LYS | A | 274 | 33.980 | 44.770 | 50.956 | 1.00 | 41.32 | A | C |
| ATOM | 2012 | CG | LYS | A | 274 | 33.540 | 43.877 | 49.812 | 1.00 | 48.91 | A | C |
| ATOM | 2013 | CD | LYS | A | 274 | 32.316 | 44.395 | 49.083 | 1.00 | 52.75 | A | C |
| ATOM | 2014 | CE | LYS | A | 274 | 31.898 | 43.387 | 48.022 | 1.00 | 55.44 | A | C |
| ATOM | 2015 | NZ | LYS | A | 274 | 30.931 | 43.949 | 47.045 | 1.00 | 61.58 | A | N |
| ATOM | 2016 | C | LYS | A | 274 | 35.649 | 45.201 | 52.754 | 1.00 | 37.87 | A | C |
| ATOM | 2017 | O | LYS | A | 274 | 35.128 | 44.988 | 53.851 | 1.00 | 37.68 | A | O |
| ATOM | 2018 | N | VAL | A | 275 | 36.543 | 46.158 | 52.556 | 1.00 | 35.30 | A | N |
| ATOM | 2019 | CA | VAL | A | 275 | 36.934 | 47.036 | 53.629 | 1.00 | 34.96 | A | C |
| ATOM | 2020 | CB | VAL | A | 275 | 38.433 | 46.878 | 54.002 | 1.00 | 31.90 | A | C |
| ATOM | 2021 | CG1 | VAL | A | 275 | 38.834 | 47.949 | 55.009 | 1.00 | 30.16 | A | C |
| ATOM | 2022 | CG2 | VAL | A | 275 | 38.676 | 45.529 | 54.600 | 1.00 | 31.65 | A | C |
| ATOM | 2023 | C | VAL | A | 275 | 36.716 | 48.466 | 53.226 | 1.00 | 35.13 | A | C |
| ATOM | 2024 | O | VAL | A | 275 | 37.029 | 48.866 | 52.104 | 1.00 | 37.86 | A | O |
| ATOM | 2025 | N | MET | A | 276 | 36.164 | 49.239 | 54.147 | 1.00 | 34.89 | A | N |
| ATOM | 2026 | CA | MET | A | 276 | 35.959 | 50.647 | 53.909 | 1.00 | 36.42 | A | C |
| ATOM | 2027 | CB | MET | A | 276 | 34.483 | 51.022 | 53.960 | 1.00 | 39.09 | A | C |
| ATOM | 2028 | CG | MET | A | 276 | 34.265 | 52.464 | 53.546 | 1.00 | 47.33 | A | C |
| ATOM | 2029 | SD | MET | A | 276 | 32.565 | 52.990 | 53.686 | 1.00 | 52.57 | A | S |
| ATOM | 2030 | CE | MET | A | 276 | 32.748 | 54.253 | 54.843 | 1.00 | 53.28 | A | C |
| ATOM | 2031 | C | MET | A | 276 | 36.700 | 51.385 | 55.008 | 1.00 | 34.59 | A | C |
| ATOM | 2032 | O | MET | A | 276 | 36.624 | 51.020 | 56.192 | 1.00 | 33.70 | A | O |
| ATOM | 2033 | N | VAL | A | 277 | 37.441 | 52.402 | 54.601 | 1.00 | 30.95 | A | N |
| ATOM | 2034 | CA | VAL | A | 277 | 38.188 | 53.209 | 55.524 | 1.00 | 29.36 | A | C |
| ATOM | 2035 | CB | VAL | A | 277 | 39.695 | 53.272 | 55.150 | 1.00 | 27.38 | A | C |
| ATOM | 2036 | CG1 | VAL | A | 277 | 40.409 | 54.195 | 56.118 | 1.00 | 24.18 | A | C |
| ATOM | 2037 | CG2 | VAL | A | 277 | 40.319 | 51.860 | 55.176 | 1.00 | 21.80 | A | C |
| ATOM | 2038 | C | VAL | A | 277 | 37.557 | 54.562 | 55.360 | 1.00 | 29.59 | A | C |
| ATOM | 2039 | O | VAL | A | 277 | 37.618 | 55.159 | 54.272 | 1.00 | 28.42 | A | O |
| ATOM | 2040 | N | ALA | A | 278 | 36.914 | 55.027 | 56.425 | 1.00 | 30.06 | A | N |
| ATOM | 2041 | CA | ALA | A | 278 | 36.248 | 56.317 | 56.399 | 1.00 | 29.96 | A | C |
| ATOM | 2042 | CB | ALA | A | 278 | 34.778 | 56.151 | 56.730 | 1.00 | 24.25 | A | C |
| ATOM | 2043 | C | ALA | A | 278 | 36.902 | 57.253 | 57.402 | 1.00 | 31.49 | A | C |
| ATOM | 2044 | O | ALA | A | 278 | 37.200 | 56.856 | 58.532 | 1.00 | 33.32 | A | O |
| ATOM | 2045 | N | LEU | A | 279 | 37.132 | 58.489 | 56.979 | 1.00 | 33.46 | A | N |
| ATOM | 2046 | CA | LEU | A | 279 | 37.727 | 59.494 | 57.839 | 1.00 | 33.54 | A | C |
| ATOM | 2047 | CB | LEU | A | 279 | 39.043 | 59.977 | 57.237 | 1.00 | 33.43 | A | C |
| ATOM | 2048 | CG | LEU | A | 279 | 39.600 | 61.273 | 57.824 | 1.00 | 37.51 | A | C |
| ATOM | 2049 | CD1 | LEU | A | 279 | 39.853 | 61.081 | 59.314 | 1.00 | 39.60 | A | C |
| ATOM | 2050 | CD2 | LEU | A | 279 | 40.889 | 61.667 | 57.099 | 1.00 | 35.34 | A | C |
| ATOM | 2051 | C | LEU | A | 279 | 36.759 | 60.657 | 58.002 | 1.00 | 35.93 | A | C |
| ATOM | 2052 | O | LEU | A | 279 | 36.422 | 61.337 | 57.026 | 1.00 | 40.89 | A | O |
| ATOM | 2053 | N | GLY | A | 280 | 36.299 | 60.875 | 59.230 | 1.00 | 37.54 | A | N |
| ATOM | 2054 | CA | GLY | A | 280 | 35.382 | 61.964 | 59.530 | 1.00 | 39.11 | A | C |
| ATOM | 2055 | C | GLY | A | 280 | 36.085 | 63.030 | 60.357 | 1.00 | 42.58 | A | C |
| ATOM | 2056 | O | GLY | A | 280 | 37.306 | 62.966 | 60.490 | 1.00 | 43.04 | A | O |
| ATOM | 2057 | N | GLU | A | 281 | 35.343 | 63.992 | 60.916 | 1.00 | 45.48 | A | N |
| ATOM | 2058 | CA | GLU | A | 281 | 35.951 | 65.061 | 61.720 | 1.00 | 50.64 | A | C |
| ATOM | 2059 | CB | GLU | A | 281 | 34.911 | 66.091 | 62.182 | 1.00 | 58.24 | A | C |
| ATOM | 2060 | CG | GLU | A | 281 | 34.318 | 66.963 | 61.087 | 1.00 | 72.99 | A | C |
| ATOM | 2061 | CD | GLU | A | 281 | 33.543 | 68.150 | 61.652 | 1.00 | 81.17 | A | C |
| ATOM | 2062 | OE1 | GLU | A | 281 | 33.100 | 68.083 | 62.824 | 1.00 | 84.06 | A | O |
| ATOM | 2063 | OE2 | GLU | A | 281 | 33.370 | 69.148 | 60.919 | 1.00 | 87.24 | A | O |
| ATOM | 2064 | C | GLU | A | 281 | 36.695 | 64.558 | 62.955 | 1.00 | 48.26 | A | C |
| ATOM | 2065 | O | GLU | A | 281 | 37.716 | 65.118 | 63.336 | 1.00 | 47.03 | A | O |
| ATOM | 2066 | N | GLU | A | 282 | 36.166 | 63.529 | 63.604 | 1.00 | 46.12 | A | N |
| ATOM | 2067 | CA | GLU | A | 282 | 36.846 | 62.999 | 64.772 | 1.00 | 47.04 | A | C |
| ATOM | 2068 | CB | GLU | A | 282 | 36.113 | 63.378 | 66.056 | 1.00 | 46.76 | A | C |
| ATOM | 2069 | CG | GLU | A | 282 | 34.700 | 62.908 | 66.168 | 1.00 | 48.63 | A | C |
| ATOM | 2070 | CD | GLU | A | 282 | 34.129 | 63.231 | 67.540 | 1.00 | 53.64 | A | C |
| ATOM | 2071 | OE1 | GLU | A | 282 | 34.601 | 62.627 | 68.527 | 1.00 | 50.75 | A | O |
| ATOM | 2072 | OE2 | GLU | A | 282 | 33.228 | 64.096 | 67.634 | 1.00 | 53.96 | A | O |
| ATOM | 2073 | C | GLU | A | 282 | 37.087 | 61.491 | 64.750 | 1.00 | 44.16 | A | C |
| ATOM | 2074 | O | GLU | A | 282 | 37.959 | 60.994 | 65.461 | 1.00 | 44.13 | A | O |
| ATOM | 2075 | N | ASP | A | 283 | 36.327 | 60.774 | 63.932 | 1.00 | 40.73 | A | N |
| ATOM | 2076 | CA | ASP | A | 283 | 36.471 | 59.328 | 63.831 | 1.00 | 37.89 | A | C |
| ATOM | 2077 | CB | ASP | A | 283 | 35.104 | 58.639 | 63.794 | 1.00 | 35.38 | A | C |
| ATOM | 2078 | CG | ASP | A | 283 | 34.434 | 58.544 | 65.150 | 1.00 | 32.59 | A | C |
| ATOM | 2079 | OD1 | ASP | A | 283 | 34.997 | 59.034 | 66.153 | 1.00 | 35.73 | A | O |
| ATOM | 2080 | OD2 | ASP | A | 283 | 33.318 | 57.970 | 65.201 | 1.00 | 32.09 | A | O |
| ATOM | 2081 | C | ASP | A | 283 | 37.193 | 58.915 | 62.563 | 1.00 | 38.00 | A | C |
| ATOM | 2082 | O | ASP | A | 283 | 37.074 | 59.560 | 61.529 | 1.00 | 39.12 | A | O |
| ATOM | 2083 | N | LEU | A | 284 | 37.950 | 57.832 | 62.662 | 1.00 | 35.28 | A | N |
| ATOM | 2084 | CA | LEU | A | 284 | 38.615 | 57.236 | 61.506 | 1.00 | 31.86 | A | C |
| ATOM | 2085 | CB | LEU | A | 284 | 40.128 | 57.324 | 61.624 | 1.00 | 31.38 | A | C |
| ATOM | 2086 | CG | LEU | A | 284 | 40.831 | 56.957 | 60.308 | 1.00 | 38.16 | A | C |
| ATOM | 2087 | CD1 | LEU | A | 284 | 42.288 | 57.420 | 60.349 | 1.00 | 40.52 | A | C |
| ATOM | 2088 | CD2 | LEU | A | 284 | 40.764 | 55.450 | 60.082 | 1.00 | 36.69 | A | C |
| ATOM | 2089 | C | LEU | A | 284 | 38.111 | 55.801 | 61.655 | 1.00 | 32.86 | A | C |
| ATOM | 2090 | O | LEU | A | 284 | 38.537 | 55.078 | 62.558 | 1.00 | 33.83 | A | O |
| ATOM | 2091 | N | SER | A | 285 | 37.176 | 55.391 | 60.808 | 1.00 | 31.11 | A | N |
| ATOM | 2092 | CA | SER | A | 285 | 36.601 | 54.059 | 60.954 | 1.00 | 30.85 | A | C |
| ATOM | 2093 | CB | SER | A | 285 | 35.083 | 54.157 | 60.995 | 1.00 | 26.90 | A | C |
| ATOM | 2094 | OG | SER | A | 285 | 34.659 | 55.203 | 61.853 | 1.00 | 30.00 | A | O |
| ATOM | 2095 | C | SER | A | 285 | 36.989 | 53.081 | 59.869 | 1.00 | 33.79 | A | C |
| ATOM | 2096 | O | SER | A | 285 | 37.112 | 53.454 | 58.696 | 1.00 | 37.41 | A | O |
| ATOM | 2097 | N | ILE | A | 286 | 37.158 | 51.823 | 60.257 | 1.00 | 32.05 | A | N |
| ATOM | 2098 | CA | ILE | A | 286 | 37.520 | 50.783 | 59.314 | 1.00 | 31.93 | A | C |
| ATOM | 2099 | CB | ILE | A | 286 | 38.906 | 50.202 | 59.617 | 1.00 | 32.04 | A | C |
| ATOM | 2100 | CG2 | ILE | A | 286 | 39.366 | 49.333 | 58.463 | 1.00 | 29.31 | A | C |
| ATOM | 2101 | CG1 | ILE | A | 286 | 39.907 | 51.350 | 59.836 | 1.00 | 37.55 | A | C |
| ATOM | 2102 | CD1 | ILE | A | 286 | 41.325 | 50.906 | 60.142 | 1.00 | 37.96 | A | C |
| ATOM | 2103 | C | ILE | A | 286 | 36.511 | 49.668 | 59.427 | 1.00 | 33.65 | A | C |
| ATOM | 2104 | O | ILE | A | 286 | 36.399 | 49.022 | 60.474 | 1.00 | 34.28 | A | O |
| ATOM | 2105 | N | LYS | A | 287 | 35.749 | 49.455 | 58.361 | 1.00 | 32.56 | A | N |
| ATOM | 2106 | CA | LYS | A | 287 | 34.784 | 48.379 | 58.375 | 1.00 | 32.88 | A | C |
| ATOM | 2107 | CB | LYS | A | 287 | 33.400 | 48.850 | 57.920 | 1.00 | 30.83 | A | C |
| ATOM | 2108 | CG | LYS | A | 287 | 32.436 | 47.674 | 57.778 | 1.00 | 35.68 | A | C |
| ATOM | 2109 | CD | LYS | A | 287 | 31.014 | 47.999 | 58.220 | 1.00 | 40.03 | A | C |
| ATOM | 2110 | CE | LYS | A | 287 | 30.332 | 48.966 | 57.292 | 1.00 | 46.95 | A | C |
| ATOM | 2111 | NZ | LYS | A | 287 | 28.884 | 49.096 | 57.665 | 1.00 | 52.58 | A | N |
| ATOM | 2112 | C | LYS | A | 287 | 35.236 | 47.232 | 57.483 | 1.00 | 31.40 | A | C |
| ATOM | 2113 | O | LYS | A | 287 | 35.705 | 47.445 | 56.362 | 1.00 | 31.47 | A | O |
| ATOM | 2114 | N | MET | A | 288 | 35.112 | 46.014 | 57.992 | 1.00 | 31.85 | A | N |
| ATOM | 2115 | CA | MET | A | 288 | 35.467 | 44.838 | 57.220 | 1.00 | 32.16 | A | C |
| ATOM | 2116 | CB | MET | A | 288 | 36.571 | 44.047 | 57.911 | 1.00 | 31.92 | A | C |
| ATOM | 2117 | CG | MET | A | 288 | 37.040 | 42.863 | 57.100 | 1.00 | 32.71 | A | C |
| ATOM | 2118 | SD | MET | A | 288 | 38.418 | 42.040 | 57.890 | 1.00 | 38.31 | A | S |
| ATOM | 2119 | CE | MET | A | 288 | 37.560 | 40.860 | 58.750 | 1.00 | 37.49 | A | C |
| ATOM | 2120 | C | MET | A | 288 | 34.209 | 43.987 | 57.116 | 1.00 | 32.29 | A | C |
| ATOM | 2121 | O | MET | A | 288 | 33.707 | 43.488 | 58.140 | 1.00 | 32.02 | A | O |
| ATOM | 2122 | N | SER | A | 289 | 33.707 | 43.826 | 55.890 | 1.00 | 31.83 | A | N |
| ATOM | 2123 | CA | SER | A | 289 | 32.494 | 43.056 | 55.641 | 1.00 | 36.99 | A | C |
| ATOM | 2124 | CB | SER | A | 289 | 31.511 | 43.865 | 54.795 | 1.00 | 34.44 | A | C |
| ATOM | 2125 | OG | SER | A | 289 | 31.252 | 45.114 | 55.393 | 1.00 | 34.73 | A | O |
| ATOM | 2126 | C | SER | A | 289 | 32.786 | 41.786 | 54.898 | 1.00 | 36.81 | A | C |
| ATOM | 2127 | O | SER | A | 289 | 33.413 | 41.827 | 53.844 | 1.00 | 41.00 | A | O |
| ATOM | 2128 | N | ASP | A | 290 | 32.329 | 40.661 | 55.449 | 1.00 | 37.34 | A | N |
| ATOM | 2129 | CA | ASP | A | 290 | 32.495 | 39.360 | 54.806 | 1.00 | 38.66 | A | C |
| ATOM | 2130 | CB | ASP | A | 290 | 33.323 | 38.425 | 55.679 | 1.00 | 38.30 | A | C |
| ATOM | 2131 | CG | ASP | A | 290 | 32.651 | 38.107 | 57.001 | 1.00 | 40.66 | A | C |
| ATOM | 2132 | OD1 | ASP | A | 290 | 31.567 | 37.467 | 57.020 | 1.00 | 37.23 | A | O |
| ATOM | 2133 | OD2 | ASP | A | 290 | 33.230 | 38.497 | 58.035 | 1.00 | 41.57 | A | O |
| ATOM | 2134 | C | ASP | A | 290 | 31.117 | 38.740 | 54.598 | 1.00 | 39.59 | A | C |
| ATOM | 2135 | O | ASP | A | 290 | 30.138 | 39.180 | 55.212 | 1.00 | 40.32 | A | O |
| ATOM | 2136 | N | ARG | A | 291 | 31.038 | 37.728 | 53.736 | 1.00 | 39.07 | A | N |
| ATOM | 2137 | CA | ARG | A | 291 | 29.786 | 37.021 | 53.478 | 1.00 | 39.45 | A | C |
| ATOM | 2138 | CB | ARG | A | 291 | 29.424 | 37.082 | 51.997 | 1.00 | 42.20 | A | C |
| ATOM | 2139 | CG | ARG | A | 291 | 29.097 | 38.484 | 51.548 | 1.00 | 48.96 | A | C |
| ATOM | 2140 | CD | ARG | A | 291 | 28.664 | 38.563 | 50.100 | 1.00 | 57.55 | A | C |
| ATOM | 2141 | NE | ARG | A | 291 | 28.482 | 39.964 | 49.711 | 1.00 | 67.36 | A | N |
| ATOM | 2142 | CZ | ARG | A | 291 | 29.450 | 40.882 | 49.716 | 1.00 | 72.14 | A | C |
| ATOM | 2143 | NH1 | ARG | A | 291 | 30.688 | 40.554 | 50.088 | 1.00 | 73.62 | A | N |
| ATOM | 2144 | NH2 | ARG | A | 291 | 29.179 | 42.134 | 49.352 | 1.00 | 73.69 | A | N |
| ATOM | 2145 | C | ARG | A | 291 | 30.009 | 35.582 | 53.924 | 1.00 | 40.00 | A | C |
| ATOM | 2146 | O | ARG | A | 291 | 29.617 | 34.629 | 53.239 | 1.00 | 39.09 | A | O |
| ATOM | 2147 | N | GLY | A | 292 | 30.640 | 35.455 | 55.096 | 1.00 | 37.64 | A | N |
| ATOM | 2148 | CA | GLY | A | 292 | 30.982 | 34.163 | 55.662 | 1.00 | 36.40 | A | C |
| ATOM | 2149 | C | GLY | A | 292 | 29.898 | 33.409 | 56.395 | 1.00 | 35.85 | A | C |
| ATOM | 2150 | O | GLY | A | 292 | 30.204 | 32.575 | 57.257 | 1.00 | 36.77 | A | O |
| ATOM | 2151 | N | GLY | A | 293 | 28.638 | 33.683 | 56.069 | 1.00 | 35.43 | A | N |
| ATOM | 2152 | CA | GLY | A | 293 | 27.553 | 32.975 | 56.725 | 1.00 | 35.57 | A | C |
| ATOM | 2153 | C | GLY | A | 293 | 26.959 | 33.706 | 57.913 | 1.00 | 37.90 | A | C |
| ATOM | 2154 | O | GLY | A | 293 | 25.768 | 33.577 | 58.193 | 1.00 | 41.02 | A | O |
| ATOM | 2155 | N | GLY | A | 294 | 27.775 | 34.470 | 58.623 | 1.00 | 34.19 | A | N |
| ATOM | 2156 | CA | GLY | A | 294 | 27.254 | 35.191 | 59.757 | 1.00 | 37.49 | A | C |
| ATOM | 2157 | C | GLY | A | 294 | 27.226 | 34.332 | 60.993 | 1.00 | 39.04 | A | C |
| ATOM | 2158 | O | GLY | A | 294 | 27.517 | 33.135 | 60.928 | 1.00 | 41.09 | A | O |
| ATOM | 2159 | N | VAL | A | 295 | 26.876 | 34.951 | 62.119 | 1.00 | 39.49 | A | N |
| ATOM | 2160 | CA | VAL | A | 295 | 26.803 | 34.272 | 63.404 | 1.00 | 38.22 | A | C |
| ATOM | 2161 | CB | VAL | A | 295 | 28.059 | 34.584 | 64.225 | 1.00 | 38.55 | A | C |
| ATOM | 2162 | CG1 | VAL | A | 295 | 27.891 | 34.078 | 65.640 | 1.00 | 40.43 | A | C |
| ATOM | 2163 | CG2 | VAL | A | 295 | 29.282 | 33.957 | 63.563 | 1.00 | 38.77 | A | C |
| ATOM | 2164 | C | VAL | A | 295 | 25.556 | 34.727 | 64.183 | 1.00 | 40.25 | A | C |
| ATOM | 2165 | O | VAL | A | 295 | 25.190 | 35.899 | 64.150 | 1.00 | 39.29 | A | O |
| ATOM | 2166 | N | PRO | A | 296 | 24.891 | 33.804 | 64.898 | 1.00 | 41.13 | A | N |
| ATOM | 2167 | CD | PRO | A | 296 | 25.278 | 32.415 | 65.181 | 1.00 | 39.35 | A | C |
| ATOM | 2168 | CA | PRO | A | 296 | 23.696 | 34.172 | 65.663 | 1.00 | 41.68 | A | C |
| ATOM | 2169 | CB | PRO | A | 296 | 23.293 | 32.862 | 66.340 | 1.00 | 42.94 | A | C |
| ATOM | 2170 | CG | PRO | A | 296 | 23.978 | 31.805 | 65.542 | 1.00 | 42.93 | A | C |
| ATOM | 2171 | C | PRO | A | 296 | 24.040 | 35.230 | 66.704 | 1.00 | 42.64 | A | C |
| ATOM | 2172 | O | PRO | A | 296 | 25.089 | 35.142 | 67.358 | 1.00 | 42.30 | A | O |
| ATOM | 2173 | N | LEU | A | 297 | 23.162 | 36.214 | 66.881 | 1.00 | 40.59 | A | N |
| ATOM | 2174 | CA | LEU | A | 297 | 23.418 | 37.264 | 67.868 | 1.00 | 42.08 | A | C |
| ATOM | 2175 | CB | LEU | A | 297 | 22.178 | 38.146 | 68.093 | 1.00 | 40.15 | A | C |
| ATOM | 2176 | CG | LEU | A | 297 | 22.451 | 39.323 | 69.050 | 1.00 | 41.16 | A | C |
| ATOM | 2177 | CD1 | LEU | A | 297 | 23.538 | 40.231 | 68.435 | 1.00 | 37.69 | A | C |
| ATOM | 2178 | CD2 | LEU | A | 297 | 21.157 | 40.110 | 69.329 | 1.00 | 38.19 | A | C |
| ATOM | 2179 | C | LEU | A | 297 | 23.864 | 36.722 | 69.221 | 1.00 | 41.04 | A | C |
| ATOM | 2180 | O | LEU | A | 297 | 24.728 | 37.305 | 69.856 | 1.00 | 41.51 | A | O |
| ATOM | 2181 | N | ARG | A | 298 | 23.289 | 35.610 | 69.668 | 1.00 | 44.59 | A | N |
| ATOM | 2182 | CA | ARG | A | 298 | 23.677 | 35.069 | 70.971 | 1.00 | 48.18 | A | C |
| ATOM | 2183 | CB | ARG | A | 298 | 22.744 | 33.927 | 71.414 | 1.00 | 52.71 | A | C |
| ATOM | 2184 | CG | ARG | A | 298 | 22.256 | 32.998 | 70.315 | 1.00 | 61.25 | A | C |
| ATOM | 2185 | CD | ARG | A | 298 | 21.482 | 31.818 | 70.914 | 1.00 | 64.85 | A | C |
| ATOM | 2186 | NE | ARG | A | 298 | 22.381 | 30.879 | 71.573 | 1.00 | 66.72 | A | N |
| ATOM | 2187 | CZ | ARG | A | 298 | 23.087 | 29.947 | 70.941 | 1.00 | 67.91 | A | C |
| ATOM | 2188 | NH1 | ARG | A | 298 | 22.999 | 29.807 | 69.625 | 1.00 | 66.12 | A | N |
| ATOM | 2189 | NH2 | ARG | A | 298 | 23.911 | 29.168 | 71.627 | 1.00 | 72.06 | A | N |
| ATOM | 2190 | C | ARG | A | 298 | 25.118 | 34.607 | 71.072 | 1.00 | 46.26 | A | C |
| ATOM | 2191 | O | ARG | A | 298 | 25.688 | 34.630 | 72.152 | 1.00 | 47.51 | A | O |
| ATOM | 2192 | N | LYS | A | 299 | 25.719 | 34.194 | 69.963 | 1.00 | 48.05 | A | N |
| ATOM | 2193 | CA | LYS | A | 299 | 27.108 | 33.743 | 70.010 | 1.00 | 50.47 | A | C |
| ATOM | 2194 | CB | LYS | A | 299 | 27.375 | 32.718 | 68.907 | 1.00 | 53.28 | A | C |
| ATOM | 2195 | CG | LYS | A | 299 | 26.567 | 31.439 | 69.064 | 1.00 | 60.47 | A | C |
| ATOM | 2196 | CD | LYS | A | 299 | 26.842 | 30.458 | 67.938 | 1.00 | 65.40 | A | C |
| ATOM | 2197 | CE | LYS | A | 299 | 25.957 | 29.227 | 68.053 | 1.00 | 69.52 | A | C |
| ATOM | 2198 | NZ | LYS | A | 299 | 26.051 | 28.365 | 66.837 | 1.00 | 71.72 | A | N |
| ATOM | 2199 | C | LYS | A | 299 | 28.090 | 34.904 | 69.878 | 1.00 | 51.09 | A | C |
| ATOM | 2200 | O | LYS | A | 299 | 29.249 | 34.790 | 70.283 | 1.00 | 50.74 | A | O |
| ATOM | 2201 | N | ILE | A | 300 | 27.639 | 36.022 | 69.310 | 1.00 | 53.30 | A | N |
| ATOM | 2202 | CA | ILE | A | 300 | 28.517 | 37.178 | 69.147 | 1.00 | 54.91 | A | C |
| ATOM | 2203 | CB | ILE | A | 300 | 27.792 | 38.361 | 68.449 | 1.00 | 53.75 | A | C |
| ATOM | 2204 | CG2 | ILE | A | 300 | 28.582 | 39.648 | 68.647 | 1.00 | 51.86 | A | C |
| ATOM | 2205 | CG1 | ILE | A | 300 | 27.636 | 38.077 | 66.958 | 1.00 | 51.64 | A | C |
| ATOM | 2206 | CD1 | ILE | A | 300 | 28.946 | 37.949 | 66.243 | 1.00 | 52.00 | A | C |
| ATOM | 2207 | C | ILE | A | 300 | 28.984 | 37.637 | 70.516 | 1.00 | 56.77 | A | C |
| ATOM | 2208 | O | ILE | A | 300 | 30.172 | 37.822 | 70.760 | 1.00 | 53.65 | A | O |
| ATOM | 2209 | N | GLU | A | 301 | 28.020 | 37.804 | 71.407 | 1.00 | 62.99 | A | N |
| ATOM | 2210 | CA | GLU | A | 301 | 28.278 | 38.254 | 72.763 | 1.00 | 70.79 | A | C |
| ATOM | 2211 | CB | GLU | A | 301 | 26.976 | 38.168 | 73.568 | 1.00 | 78.52 | A | C |
| ATOM | 2212 | CG | GLU | A | 301 | 25.689 | 38.397 | 72.741 | 1.00 | 87.59 | A | C |
| ATOM | 2213 | CD | GLU | A | 301 | 25.385 | 39.869 | 72.456 | 1.00 | 94.51 | A | C |
| ATOM | 2214 | OE1 | GLU | A | 301 | 26.241 | 40.568 | 71.867 | 1.00 | 98.27 | A | O |
| ATOM | 2215 | OE2 | GLU | A | 301 | 24.277 | 40.327 | 72.818 | 1.00 | 97.77 | A | O |
| ATOM | 2216 | C | GLU | A | 301 | 29.370 | 37.403 | 73.422 | 1.00 | 71.51 | A | C |
| ATOM | 2217 | O | GLU | A | 301 | 30.116 | 37.882 | 74.270 | 1.00 | 73.58 | A | O |
| ATOM | 2218 | N | ARG | A | 302 | 29.469 | 36.144 | 73.008 | 1.00 | 71.43 | A | N |
| ATOM | 2219 | CA | ARG | A | 302 | 30.443 | 35.212 | 73.572 | 1.00 | 70.11 | A | C |
| ATOM | 2220 | CB | ARG | A | 302 | 29.826 | 33.810 | 73.636 | 1.00 | 76.80 | A | C |
| ATOM | 2221 | CG | ARG | A | 302 | 28.503 | 33.758 | 74.400 | 1.00 | 86.02 | A | C |
| ATOM | 2222 | CD | ARG | A | 302 | 27.802 | 32.413 | 74.209 | 1.00 | 92.26 | A | C |
| ATOM | 2223 | NE | ARG | A | 302 | 28.595 | 31.290 | 74.707 | 1.00 | 98.19 | A | N |
| ATOM | 2224 | CZ | ARG | A | 302 | 28.736 | 30.972 | 75.992 | 1.00 | 100.89 | A | C |
| ATOM | 2225 | NH1 | ARG | A | 302 | 28.132 | 31.689 | 76.930 | 1.00 | 101.78 | A | N |
| ATOM | 2226 | NH2 | ARG | A | 302 | 29.485 | 29.933 | 76.335 | 1.00 | 100.91 | A | N |
| ATOM | 2227 | C | ARG | A | 302 | 31.779 | 35.146 | 72.828 | 1.00 | 66.40 | A | C |
| ATOM | 2228 | O | ARG | A | 302 | 32.702 | 34.451 | 73.267 | 1.00 | 64.49 | A | O |
| ATOM | 2229 | N | LEU | A | 303 | 31.891 | 35.857 | 71.710 | 1.00 | 60.05 | A | N |
| ATOM | 2230 | CA | LEU | A | 303 | 33.130 | 35.858 | 70.946 | 1.00 | 53.02 | A | C |
| ATOM | 2231 | CB | LEU | A | 303 | 32.879 | 36.356 | 69.512 | 1.00 | 51.35 | A | C |
| ATOM | 2232 | CG | LEU | A | 303 | 32.162 | 35.445 | 68.516 | 1.00 | 47.80 | A | C |
| ATOM | 2233 | CD1 | LEU | A | 303 | 31.960 | 36.189 | 67.206 | 1.00 | 46.19 | A | C |
| ATOM | 2234 | CD2 | LEU | A | 303 | 32.991 | 34.175 | 68.298 | 1.00 | 46.06 | A | C |
| ATOM | 2235 | C | LEU | A | 303 | 34.218 | 36.716 | 71.593 | 1.00 | 50.64 | A | C |
| ATOM | 2236 | O | LEU | A | 303 | 35.403 | 36.511 | 71.329 | 1.00 | 50.59 | A | O |
| ATOM | 2237 | N | PHE | A | 304 | 33.830 | 37.680 | 72.422 | 1.00 | 46.30 | A | N |
| ATOM | 2238 | CA | PHE | A | 304 | 34.820 | 38.539 | 73.074 | 1.00 | 46.94 | A | C |
| ATOM | 2239 | CB | PHE | A | 304 | 34.352 | 40.007 | 73.097 | 1.00 | 44.10 | A | C |
| ATOM | 2240 | CG | PHE | A | 304 | 34.381 | 40.692 | 71.742 | 1.00 | 42.46 | A | C |
| ATOM | 2241 | CD1 | PHE | A | 304 | 33.247 | 40.717 | 70.929 | 1.00 | 37.85 | A | C |
| ATOM | 2242 | CD2 | PHE | A | 304 | 35.535 | 41.344 | 71.300 | 1.00 | 39.76 | A | C |
| ATOM | 2243 | CE1 | PHE | A | 304 | 33.262 | 41.393 | 69.699 | 1.00 | 38.78 | A | C |
| ATOM | 2244 | CE2 | PHE | A | 304 | 35.557 | 42.019 | 70.065 | 1.00 | 39.57 | A | C |
| ATOM | 2245 | CZ | PHE | A | 304 | 34.416 | 42.044 | 69.272 | 1.00 | 35.49 | A | C |
| ATOM | 2246 | C | PHE | A | 304 | 35.120 | 38.084 | 74.507 | 1.00 | 46.86 | A | C |
| ATOM | 2247 | O | PHE | A | 304 | 35.924 | 38.694 | 75.212 | 1.00 | 46.24 | A | O |
| ATOM | 2248 | N | SER | A | 305 | 34.479 | 37.003 | 74.931 | 1.00 | 47.49 | A | N |
| ATOM | 2249 | CA | SER | A | 305 | 34.679 | 36.487 | 76.279 | 1.00 | 50.00 | A | C |
| ATOM | 2250 | CB | SER | A | 305 | 33.462 | 35.674 | 76.692 | 1.00 | 51.72 | A | C |
| ATOM | 2251 | OG | SER | A | 305 | 33.688 | 35.036 | 77.932 | 1.00 | 55.79 | A | O |
| ATOM | 2252 | C | SER | A | 305 | 35.936 | 35.623 | 76.400 | 1.00 | 49.70 | A | C |
| ATOM | 2253 | O | SER | A | 305 | 36.116 | 34.676 | 75.651 | 1.00 | 49.12 | A | O |
| ATOM | 2254 | N | TYR | A | 306 | 36.811 | 35.948 | 77.339 | 1.00 | 51.82 | A | N |
| ATOM | 2255 | CA | TYR | A | 306 | 38.026 | 35.161 | 77.513 | 1.00 | 55.36 | A | C |
| ATOM | 2256 | CB | TYR | A | 306 | 38.943 | 35.777 | 78.572 | 1.00 | 52.68 | A | C |
| ATOM | 2257 | CG | TYR | A | 306 | 39.444 | 37.150 | 78.230 | 1.00 | 51.19 | A | C |
| ATOM | 2258 | CD1 | TYR | A | 306 | 38.822 | 38.283 | 78.745 | 1.00 | 49.91 | A | C |
| ATOM | 2259 | CE1 | TYR | A | 306 | 39.274 | 39.560 | 78.422 | 1.00 | 50.11 | A | C |
| ATOM | 2260 | CD2 | TYR | A | 306 | 40.532 | 37.319 | 77.378 | 1.00 | 47.06 | A | C |
| ATOM | 2261 | CE2 | TYR | A | 306 | 40.989 | 38.586 | 77.048 | 1.00 | 51.55 | A | C |
| ATOM | 2262 | CZ | TYR | A | 306 | 40.354 | 39.702 | 77.577 | 1.00 | 49.49 | A | C |
| ATOM | 2263 | OH | TYR | A | 306 | 40.802 | 40.960 | 77.264 | 1.00 | 52.45 | A | O |
| ATOM | 2264 | C | TYR | A | 306 | 37.703 | 33.743 | 77.948 | 1.00 | 59.44 | A | C |
| ATOM | 2265 | O | TYR | A | 306 | 38.293 | 32.787 | 77.457 | 1.00 | 59.46 | A | O |
| ATOM | 2266 | N | MET | A | 307 | 36.768 | 33.614 | 78.882 | 1.00 | 64.42 | A | N |
| ATOM | 2267 | CA | MET | A | 307 | 36.403 | 32.308 | 79.401 | 1.00 | 70.78 | A | C |
| ATOM | 2268 | CB | MET | A | 307 | 35.472 | 32.455 | 80.605 | 1.00 | 71.64 | A | C |
| ATOM | 2269 | CG | MET | A | 307 | 35.144 | 31.128 | 81.253 | 1.00 | 75.35 | A | C |
| ATOM | 2270 | SD | MET | A | 307 | 34.418 | 31.299 | 82.878 | 1.00 | 81.16 | A | S |
| ATOM | 2271 | CE | MET | A | 307 | 32.691 | 31.533 | 82.456 | 1.00 | 81.07 | A | C |
| ATOM | 2272 | C | MET | A | 307 | 35.765 | 31.387 | 78.371 | 1.00 | 74.30 | A | C |
| ATOM | 2273 | O | MET | A | 307 | 36.200 | 30.248 | 78.196 | 1.00 | 74.24 | A | O |
| ATOM | 2274 | N | TYR | A | 308 | 34.733 | 31.876 | 77.693 | 1.00 | 78.48 | A | N |
| ATOM | 2275 | CA | TYR | A | 308 | 34.033 | 31.078 | 76.696 | 1.00 | 82.61 | A | C |
| ATOM | 2276 | CB | TYR | A | 308 | 32.709 | 31.749 | 76.333 | 1.00 | 82.93 | A | C |
| ATOM | 2277 | CG | TYR | A | 308 | 31.794 | 31.932 | 77.519 | 1.00 | 84.61 | A | C |
| ATOM | 2278 | CD1 | TYR | A | 308 | 31.170 | 33.155 | 77.756 | 1.00 | 86.74 | A | C |
| ATOM | 2279 | CE1 | TYR | A | 308 | 30.326 | 33.336 | 78.851 | 1.00 | 87.37 | A | C |
| ATOM | 2280 | CD2 | TYR | A | 308 | 31.551 | 30.885 | 78.409 | 1.00 | 85.45 | A | C |
| ATOM | 2281 | CE2 | TYR | A | 308 | 30.706 | 31.053 | 79.506 | 1.00 | 87.42 | A | C |
| ATOM | 2282 | CZ | TYR | A | 308 | 30.099 | 32.284 | 79.720 | 1.00 | 87.80 | A | C |
| ATOM | 2283 | OH | TYR | A | 308 | 29.269 | 32.463 | 80.804 | 1.00 | 88.99 | A | O |
| ATOM | 2284 | C | TYR | A | 308 | 34.852 | 30.825 | 75.435 | 1.00 | 85.32 | A | C |
| ATOM | 2285 | O | TYR | A | 308 | 34.586 | 29.875 | 74.703 | 1.00 | 85.82 | A | O |
| ATOM | 2286 | N | SER | A | 309 | 35.850 | 31.665 | 75.181 | 1.00 | 89.10 | A | N |
| ATOM | 2287 | CA | SER | A | 309 | 36.684 | 31.493 | 73.999 | 1.00 | 94.44 | A | C |
| ATOM | 2288 | CB | SER | A | 309 | 37.031 | 32.853 | 73.382 | 1.00 | 93.94 | A | C |
| ATOM | 2289 | OG | SER | A | 309 | 37.906 | 33.592 | 74.217 | 1.00 | 93.51 | A | O |
| ATOM | 2290 | C | SER | A | 309 | 37.968 | 30.736 | 74.335 | 1.00 | 98.97 | A | C |
| ATOM | 2291 | O | SER | A | 309 | 38.970 | 30.843 | 73.624 | 1.00 | 99.27 | A | O |
| ATOM | 2292 | N | THR | A | 310 | 37.931 | 29.966 | 75.420 | 1.00 | 104.69 | A | N |
| ATOM | 2293 | CA | THR | A | 310 | 39.088 | 29.189 | 75.852 | 1.00 | 110.88 | A | C |
| ATOM | 2294 | CB | THR | A | 310 | 39.528 | 29.586 | 77.279 | 1.00 | 109.80 | A | C |
| ATOM | 2295 | OG1 | THR | A | 310 | 39.954 | 30.953 | 77.283 | 1.00 | 109.29 | A | O |
| ATOM | 2296 | CG2 | THR | A | 310 | 40.679 | 28.710 | 77.750 | 1.00 | 109.54 | A | C |
| ATOM | 2297 | C | THR | A | 310 | 38.795 | 27.694 | 75.825 | 1.00 | 115.97 | A | C |
| ATOM | 2298 | O | THR | A | 310 | 37.694 | 27.257 | 76.156 | 1.00 | 116.28 | A | O |
| ATOM | 2299 | N | ALA | A | 311 | 39.796 | 26.917 | 75.429 | 1.00 | 122.91 | A | N |
| ATOM | 2300 | CA | ALA | A | 311 | 39.669 | 25.468 | 75.343 | 1.00 | 129.96 | A | C |
| ATOM | 2301 | CB | ALA | A | 311 | 38.904 | 25.096 | 74.072 | 1.00 | 129.97 | A | C |
| ATOM | 2302 | C | ALA | A | 311 | 41.065 | 24.838 | 75.334 | 1.00 | 134.83 | A | C |
| ATOM | 2303 | O | ALA | A | 311 | 42.072 | 25.549 | 75.334 | 1.00 | 135.76 | A | O |
| ATOM | 2304 | N | PRO | A | 312 | 41.147 | 23.495 | 75.330 | 1.00 | 139.01 | A | N |
| ATOM | 2305 | CD | PRO | A | 312 | 42.423 | 22.822 | 75.039 | 1.00 | 140.59 | A | C |
| ATOM | 2306 | CA | PRO | A | 312 | 40.049 | 22.521 | 75.335 | 1.00 | 141.50 | A | C |
| ATOM | 2307 | CB | PRO | A | 312 | 40.767 | 21.193 | 75.111 | 1.00 | 142.39 | A | C |
| ATOM | 2308 | CG | PRO | A | 312 | 41.964 | 21.593 | 74.303 | 1.00 | 142.21 | A | C |
| ATOM | 2309 | C | PRO | A | 312 | 39.253 | 22.533 | 76.637 | 1.00 | 142.90 | A | C |
| ATOM | 2310 | O | PRO | A | 312 | 38.023 | 22.511 | 76.622 | 1.00 | 144.26 | A | O |
| ATOM | 2311 | N | GLY | A | 327 | 41.653 | 31.410 | 69.951 | 1.00 | 87.70 | A | N |
| ATOM | 2312 | CA | GLY | A | 327 | 42.525 | 32.184 | 70.816 | 1.00 | 85.62 | A | C |
| ATOM | 2313 | C | GLY | A | 327 | 41.856 | 33.445 | 71.329 | 1.00 | 83.94 | A | C |
| ATOM | 2314 | O | GLY | A | 327 | 40.626 | 33.549 | 71.316 | 1.00 | 84.13 | A | O |
| ATOM | 2315 | N | TYR | A | 328 | 42.662 | 34.404 | 71.781 | 1.00 | 80.66 | A | N |
| ATOM | 2316 | CA | TYR | A | 328 | 42.139 | 35.672 | 72.298 | 1.00 | 76.91 | A | C |
| ATOM | 2317 | CB | TYR | A | 328 | 42.829 | 36.045 | 73.618 | 1.00 | 79.82 | A | C |
| ATOM | 2318 | CG | TYR | A | 328 | 42.553 | 35.110 | 74.773 | 1.00 | 82.34 | A | C |
| ATOM | 2319 | CD1 | TYR | A | 328 | 41.305 | 34.502 | 74.926 | 1.00 | 84.09 | A | C |
| ATOM | 2320 | CE1 | TYR | A | 328 | 41.034 | 33.674 | 76.019 | 1.00 | 85.26 | A | C |
| ATOM | 2321 | CD2 | TYR | A | 328 | 43.527 | 34.868 | 75.744 | 1.00 | 84.35 | A | C |
| ATOM | 2322 | CE2 | TYR | A | 328 | 43.265 | 34.049 | 76.840 | 1.00 | 84.53 | A | C |
| ATOM | 2323 | CZ | TYR | A | 328 | 42.020 | 33.455 | 76.972 | 1.00 | 84.93 | A | C |
| ATOM | 2324 | OH | TYR | A | 328 | 41.766 | 32.648 | 78.057 | 1.00 | 85.11 | A | O |
| ATOM | 2325 | C | TYR | A | 328 | 42.295 | 36.844 | 71.319 | 1.00 | 71.11 | A | C |
| ATOM | 2326 | O | TYR | A | 328 | 42.348 | 38.002 | 71.744 | 1.00 | 69.85 | A | O |
| ATOM | 2327 | N | GLY | A | 329 | 42.362 | 36.534 | 70.022 | 1.00 | 63.18 | A | N |
| ATOM | 2328 | CA | GLY | A | 329 | 42.517 | 37.553 | 69.000 | 1.00 | 53.90 | A | C |
| ATOM | 2329 | C | GLY | A | 329 | 41.518 | 38.693 | 69.121 | 1.00 | 50.89 | A | C |
| ATOM | 2330 | O | GLY | A | 329 | 41.902 | 39.862 | 69.140 | 1.00 | 48.38 | A | O |
| ATOM | 2331 | N | LEU | A | 330 | 40.236 | 38.353 | 69.219 | 1.00 | 46.34 | A | N |
| ATOM | 2332 | CA | LEU | A | 330 | 39.190 | 39.352 | 69.316 | 1.00 | 45.96 | A | C |
| ATOM | 2333 | CB | LEU | A | 330 | 37.819 | 38.699 | 69.131 | 1.00 | 43.35 | A | C |
| ATOM | 2334 | CG | LEU | A | 330 | 37.415 | 38.577 | 67.663 | 1.00 | 46.62 | A | C |
| ATOM | 2335 | CD1 | LEU | A | 330 | 36.065 | 37.845 | 67.538 | 1.00 | 44.33 | A | C |
| ATOM | 2336 | CD2 | LEU | A | 330 | 37.355 | 39.990 | 67.054 | 1.00 | 43.26 | A | C |
| ATOM | 2337 | C | LEU | A | 330 | 39.194 | 40.199 | 70.593 | 1.00 | 45.51 | A | C |
| ATOM | 2338 | O | LEU | A | 330 | 39.381 | 41.415 | 70.531 | 1.00 | 42.51 | A | O |
| ATOM | 2339 | N | PRO | A | 331 | 39.008 | 39.570 | 71.766 | 1.00 | 45.05 | A | N |
| ATOM | 2340 | CD | PRO | A | 331 | 38.839 | 38.137 | 72.074 | 1.00 | 43.58 | A | C |
| ATOM | 2341 | CA | PRO | A | 331 | 39.003 | 40.386 | 72.985 | 1.00 | 43.37 | A | C |
| ATOM | 2342 | CB | PRO | A | 331 | 38.852 | 39.345 | 74.111 | 1.00 | 44.50 | A | C |
| ATOM | 2343 | CG | PRO | A | 331 | 39.340 | 38.055 | 73.487 | 1.00 | 45.34 | A | C |
| ATOM | 2344 | C | PRO | A | 331 | 40.217 | 41.296 | 73.146 | 1.00 | 41.81 | A | C |
| ATOM | 2345 | O | PRO | A | 331 | 40.087 | 42.445 | 73.568 | 1.00 | 41.99 | A | O |
| ATOM | 2346 | N | ILE | A | 332 | 41.390 | 40.799 | 72.782 | 1.00 | 39.65 | A | N |
| ATOM | 2347 | CA | ILE | A | 332 | 42.598 | 41.594 | 72.901 | 1.00 | 38.43 | A | C |
| ATOM | 2348 | CB | ILE | A | 332 | 43.854 | 40.697 | 72.921 | 1.00 | 36.44 | A | C |
| ATOM | 2349 | CG2 | ILE | A | 332 | 45.112 | 41.528 | 72.710 | 1.00 | 30.15 | A | C |
| ATOM | 2350 | CG1 | ILE | A | 332 | 43.933 | 39.970 | 74.267 | 1.00 | 34.97 | A | C |
| ATOM | 2351 | CD1 | ILE | A | 332 | 45.138 | 39.030 | 74.389 | 1.00 | 39.58 | A | C |
| ATOM | 2352 | C | ILE | A | 332 | 42.722 | 42.630 | 71.791 | 1.00 | 38.98 | A | C |
| ATOM | 2353 | O | ILE | A | 332 | 43.251 | 43.718 | 72.023 | 1.00 | 42.11 | A | O |
| ATOM | 2354 | N | SER | A | 333 | 42.265 | 42.303 | 70.584 | 1.00 | 36.80 | A | N |
| ATOM | 2355 | CA | SER | A | 333 | 42.326 | 43.276 | 69.505 | 1.00 | 36.19 | A | C |
| ATOM | 2356 | CB | SER | A | 333 | 41.735 | 42.699 | 68.222 | 1.00 | 34.18 | A | C |
| ATOM | 2357 | OG | SER | A | 333 | 42.623 | 41.764 | 67.649 | 1.00 | 36.86 | A | O |
| ATOM | 2358 | C | SER | A | 333 | 41.499 | 44.483 | 69.967 | 1.00 | 36.64 | A | C |
| ATOM | 2359 | O | SER | A | 333 | 41.898 | 45.628 | 69.787 | 1.00 | 32.67 | A | O |
| ATOM | 2360 | N | ARG | A | 334 | 40.355 | 44.215 | 70.589 | 1.00 | 34.81 | A | N |
| ATOM | 2361 | CA | ARG | A | 334 | 39.500 | 45.285 | 71.067 | 1.00 | 37.48 | A | C |
| ATOM | 2362 | CB | ARG | A | 334 | 38.244 | 44.707 | 71.685 | 1.00 | 36.67 | A | C |
| ATOM | 2363 | CG | ARG | A | 334 | 37.277 | 45.767 | 72.139 | 1.00 | 40.22 | A | C |
| ATOM | 2364 | CD | ARG | A | 334 | 36.400 | 45.236 | 73.238 | 1.00 | 42.28 | A | C |
| ATOM | 2365 | NE | ARG | A | 334 | 35.073 | 44.862 | 72.784 | 1.00 | 43.58 | A | N |
| ATOM | 2366 | CZ | ARG | A | 334 | 34.279 | 44.020 | 73.442 | 1.00 | 44.68 | A | C |
| ATOM | 2367 | NH1 | ARG | A | 334 | 34.693 | 43.467 | 74.570 | 1.00 | 42.58 | A | N |
| ATOM | 2368 | NH2 | ARG | A | 334 | 33.065 | 43.745 | 72.980 | 1.00 | 47.09 | A | N |
| ATOM | 2369 | C | ARG | A | 334 | 40.211 | 46.179 | 72.098 | 1.00 | 39.88 | A | C |
| ATOM | 2370 | O | ARG | A | 334 | 40.057 | 47.403 | 72.082 | 1.00 | 39.44 | A | O |
| ATOM | 2371 | N | LEU | A | 335 | 40.980 | 45.567 | 72.996 | 1.00 | 39.09 | A | N |
| ATOM | 2372 | CA | LEU | A | 335 | 41.714 | 46.327 | 73.990 | 1.00 | 35.29 | A | C |
| ATOM | 2373 | CB | LEU | A | 335 | 42.475 | 45.398 | 74.941 | 1.00 | 34.73 | A | C |
| ATOM | 2374 | CG | LEU | A | 335 | 41.582 | 44.557 | 75.865 | 1.00 | 35.20 | A | C |
| ATOM | 2375 | CD1 | LEU | A | 335 | 42.428 | 43.736 | 76.812 | 1.00 | 33.60 | A | C |
| ATOM | 2376 | CD2 | LEU | A | 335 | 40.667 | 45.462 | 76.658 | 1.00 | 35.77 | A | C |
| ATOM | 2377 | C | LEU | A | 335 | 42.684 | 47.257 | 73.273 | 1.00 | 34.96 | A | C |
| ATOM | 2378 | O | LEU | A | 335 | 42.867 | 48.391 | 73.702 | 1.00 | 35.02 | A | O |
| ATOM | 2379 | N | TYR | A | 336 | 43.309 | 46.806 | 72.185 | 1.00 | 34.93 | A | N |
| ATOM | 2380 | CA | TYR | A | 336 | 44.222 | 47.695 | 71.469 | 1.00 | 36.90 | A | C |
| ATOM | 2381 | CB | TYR | A | 336 | 44.908 | 46.984 | 70.312 | 1.00 | 39.98 | A | C |
| ATOM | 2382 | CG | TYR | A | 336 | 46.123 | 46.204 | 70.722 | 1.00 | 45.98 | A | C |
| ATOM | 2383 | CD1 | TYR | A | 336 | 46.043 | 44.831 | 70.956 | 1.00 | 45.67 | A | C |
| ATOM | 2384 | CE1 | TYR | A | 336 | 47.149 | 44.113 | 71.384 | 1.00 | 47.12 | A | C |
| ATOM | 2385 | CD2 | TYR | A | 336 | 47.352 | 46.843 | 70.928 | 1.00 | 46.90 | A | C |
| ATOM | 2386 | CE2 | TYR | A | 336 | 48.470 | 46.129 | 71.362 | 1.00 | 47.30 | A | C |
| ATOM | 2387 | CZ | TYR | A | 336 | 48.354 | 44.758 | 71.590 | 1.00 | 47.99 | A | C |
| ATOM | 2388 | OH | TYR | A | 336 | 49.421 | 44.020 | 72.044 | 1.00 | 48.22 | A | O |
| ATOM | 2389 | C | TYR | A | 336 | 43.473 | 48.901 | 70.908 | 1.00 | 38.63 | A | C |
| ATOM | 2390 | O | TYR | A | 336 | 44.023 | 50.009 | 70.838 | 1.00 | 36.77 | A | O |
| ATOM | 2391 | N | ALA | A | 337 | 42.225 | 48.677 | 70.495 | 1.00 | 36.53 | A | N |
| ATOM | 2392 | CA | ALA | A | 337 | 41.406 | 49.746 | 69.935 | 1.00 | 38.62 | A | C |
| ATOM | 2393 | CB | ALA | A | 337 | 40.108 | 49.168 | 69.348 | 1.00 | 36.44 | A | C |
| ATOM | 2394 | C | ALA | A | 337 | 41.084 | 50.762 | 71.032 | 1.00 | 36.98 | A | C |
| ATOM | 2395 | O | ALA | A | 337 | 41.259 | 51.978 | 70.855 | 1.00 | 33.27 | A | O |
| ATOM | 2396 | N | LYS | A | 338 | 40.618 | 50.244 | 72.163 | 1.00 | 37.39 | A | N |
| ATOM | 2397 | CA | LYS | A | 338 | 40.238 | 51.067 | 73.312 | 1.00 | 38.22 | A | C |
| ATOM | 2398 | CB | LYS | A | 338 | 39.591 | 50.199 | 74.388 | 1.00 | 36.85 | A | C |
| ATOM | 2399 | CG | LYS | A | 338 | 38.176 | 49.757 | 74.099 | 1.00 | 38.11 | A | C |
| ATOM | 2400 | CD | LYS | A | 338 | 37.692 | 48.865 | 75.214 | 1.00 | 43.05 | A | C |
| ATOM | 2401 | CE | LYS | A | 338 | 36.366 | 48.236 | 74.871 | 1.00 | 44.64 | A | C |
| ATOM | 2402 | NZ | LYS | A | 338 | 35.328 | 49.281 | 74.749 | 1.00 | 49.35 | A | N |
| ATOM | 2403 | C | LYS | A | 338 | 41.411 | 51.807 | 73.944 | 1.00 | 37.89 | A | C |
| ATOM | 2404 | O | LYS | A | 338 | 41.241 | 52.887 | 74.496 | 1.00 | 36.36 | A | O |
| ATOM | 2405 | N | TYR | A | 339 | 42.600 | 51.221 | 73.860 | 1.00 | 35.72 | A | N |
| ATOM | 2406 | CA | TYR | A | 339 | 43.782 | 51.825 | 74.465 | 1.00 | 38.32 | A | C |
| ATOM | 2407 | CB | TYR | A | 339 | 45.033 | 51.022 | 74.072 | 1.00 | 36.27 | A | C |
| ATOM | 2408 | CG | TYR | A | 339 | 46.340 | 51.618 | 74.521 | 1.00 | 32.32 | A | C |
| ATOM | 2409 | CD1 | TYR | A | 339 | 46.517 | 52.088 | 75.825 | 1.00 | 37.44 | A | C |
| ATOM | 2410 | CE1 | TYR | A | 339 | 47.753 | 52.587 | 76.258 | 1.00 | 32.38 | A | C |
| ATOM | 2411 | CD2 | TYR | A | 339 | 47.425 | 51.662 | 73.656 | 1.00 | 36.84 | A | C |
| ATOM | 2412 | CE2 | TYR | A | 339 | 48.675 | 52.156 | 74.079 | 1.00 | 37.76 | A | C |
| ATOM | 2413 | CZ | TYR | A | 339 | 48.819 | 52.612 | 75.380 | 1.00 | 37.40 | A | C |
| ATOM | 2414 | OH | TYR | A | 339 | 50.055 | 53.075 | 75.786 | 1.00 | 43.65 | A | O |
| ATOM | 2415 | C | TYR | A | 339 | 43.934 | 53.305 | 74.101 | 1.00 | 38.15 | A | C |
| ATOM | 2416 | O | TYR | A | 339 | 44.366 | 54.114 | 74.931 | 1.00 | 35.77 | A | O |
| ATOM | 2417 | N | PHE | A | 340 | 43.589 | 53.654 | 72.864 | 1.00 | 36.48 | A | N |
| ATOM | 2418 | CA | PHE | A | 340 | 43.662 | 55.045 | 72.422 | 1.00 | 36.28 | A | C |
| ATOM | 2419 | CB | PHE | A | 340 | 44.566 | 55.173 | 71.192 | 1.00 | 36.55 | A | C |
| ATOM | 2420 | CG | PHE | A | 340 | 46.045 | 55.157 | 71.524 | 1.00 | 42.73 | A | C |
| ATOM | 2421 | CD1 | PHE | A | 340 | 46.629 | 56.230 | 72.207 | 1.00 | 44.06 | A | C |
| ATOM | 2422 | CD2 | PHE | A | 340 | 46.848 | 54.079 | 71.167 | 1.00 | 41.61 | A | C |
| ATOM | 2423 | CE1 | PHE | A | 340 | 47.992 | 56.231 | 72.524 | 1.00 | 45.05 | A | C |
| ATOM | 2424 | CE2 | PHE | A | 340 | 48.211 | 54.070 | 71.478 | 1.00 | 44.04 | A | C |
| ATOM | 2425 | CZ | PHE | A | 340 | 48.781 | 55.153 | 72.161 | 1.00 | 44.41 | A | C |
| ATOM | 2426 | C | PHE | A | 340 | 42.268 | 55.630 | 72.159 | 1.00 | 35.88 | A | C |
| ATOM | 2427 | O | PHE | A | 340 | 42.047 | 56.378 | 71.202 | 1.00 | 35.59 | A | O |
| ATOM | 2428 | N | GLN | A | 341 | 41.333 | 55.279 | 73.041 | 1.00 | 34.07 | A | N |
| ATOM | 2429 | CA | GLN | A | 341 | 39.963 | 55.777 | 72.986 | 1.00 | 35.11 | A | C |
| ATOM | 2430 | CB | GLN | A | 341 | 39.964 | 57.316 | 72.955 | 1.00 | 34.80 | A | C |
| ATOM | 2431 | CG | GLN | A | 341 | 40.731 | 58.008 | 74.065 | 1.00 | 32.62 | A | C |
| ATOM | 2432 | CD | GLN | A | 341 | 40.220 | 57.633 | 75.429 | 1.00 | 38.27 | A | C |
| ATOM | 2433 | OE1 | GLN | A | 341 | 39.007 | 57.595 | 75.660 | 1.00 | 38.48 | A | O |
| ATOM | 2434 | NE2 | GLN | A | 341 | 41.141 | 57.354 | 76.356 | 1.00 | 36.12 | A | N |
| ATOM | 2435 | C | GLN | A | 341 | 39.147 | 55.262 | 71.797 | 1.00 | 34.80 | A | C |
| ATOM | 2436 | O | GLN | A | 341 | 38.101 | 55.815 | 71.476 | 1.00 | 37.55 | A | O |
| ATOM | 2437 | N | GLY | A | 342 | 39.613 | 54.217 | 71.133 | 1.00 | 34.63 | A | N |
| ATOM | 2438 | CA | GLY | A | 342 | 38.871 | 53.703 | 70.000 | 1.00 | 31.34 | A | C |
| ATOM | 2439 | C | GLY | A | 342 | 38.041 | 52.545 | 70.470 | 1.00 | 33.75 | A | C |
| ATOM | 2440 | O | GLY | A | 342 | 37.837 | 52.401 | 71.668 | 1.00 | 36.02 | A | O |
| ATOM | 2441 | N | ASP | A | 343 | 37.568 | 51.712 | 69.544 | 1.00 | 35.25 | A | N |
| ATOM | 2442 | CA | ASP | A | 343 | 36.761 | 50.551 | 69.906 | 1.00 | 32.19 | A | C |
| ATOM | 2443 | CB | ASP | A | 343 | 35.324 | 50.995 | 70.191 | 1.00 | 30.05 | A | C |
| ATOM | 2444 | CG | ASP | A | 343 | 34.537 | 49.971 | 71.009 | 1.00 | 33.85 | A | C |
| ATOM | 2445 | OD1 | ASP | A | 343 | 35.028 | 48.840 | 71.232 | 1.00 | 34.65 | A | O |
| ATOM | 2446 | OD2 | ASP | A | 343 | 33.411 | 50.292 | 71.422 | 1.00 | 35.52 | A | O |
| ATOM | 2447 | C | ASP | A | 343 | 36.798 | 49.541 | 68.746 | 1.00 | 34.08 | A | C |
| ATOM | 2448 | O | ASP | A | 343 | 37.380 | 49.816 | 67.680 | 1.00 | 32.46 | A | O |
| ATOM | 2449 | N | LEU | A | 344 | 36.221 | 48.359 | 68.960 | 1.00 | 29.16 | A | N |
| ATOM | 2450 | CA | LEU | A | 344 | 36.174 | 47.339 | 67.912 | 1.00 | 29.35 | A | C |
| ATOM | 2451 | CB | LEU | A | 344 | 37.333 | 46.351 | 68.038 | 1.00 | 28.31 | A | C |
| ATOM | 2452 | CG | LEU | A | 344 | 37.566 | 45.413 | 66.837 | 1.00 | 30.91 | A | C |
| ATOM | 2453 | CD1 | LEU | A | 344 | 38.982 | 44.881 | 66.876 | 1.00 | 32.14 | A | C |
| ATOM | 2454 | CD2 | LEU | A | 344 | 36.563 | 44.263 | 66.851 | 1.00 | 31.07 | A | C |
| ATOM | 2455 | C | LEU | A | 344 | 34.851 | 46.640 | 68.138 | 1.00 | 31.60 | A | C |
| ATOM | 2456 | O | LEU | A | 344 | 34.616 | 46.087 | 69.215 | 1.00 | 29.55 | A | O |
| ATOM | 2457 | N | GLN | A | 345 | 33.965 | 46.673 | 67.146 | 1.00 | 29.71 | A | N |
| ATOM | 2458 | CA | GLN | A | 345 | 32.669 | 46.061 | 67.372 | 1.00 | 28.84 | A | C |
| ATOM | 2459 | CB | GLN | A | 345 | 31.596 | 47.154 | 67.486 | 1.00 | 27.81 | A | C |
| ATOM | 2460 | CG | GLN | A | 345 | 31.968 | 48.254 | 68.469 | 1.00 | 33.00 | A | C |
| ATOM | 2461 | CD | GLN | A | 345 | 30.900 | 49.352 | 68.629 | 1.00 | 37.90 | A | C |
| ATOM | 2462 | OE1 | GLN | A | 345 | 30.058 | 49.571 | 67.760 | 1.00 | 35.37 | A | O |
| ATOM | 2463 | NE2 | GLN | A | 345 | 30.961 | 50.057 | 69.752 | 1.00 | 34.99 | A | N |
| ATOM | 2464 | C | GLN | A | 345 | 32.296 | 45.095 | 66.276 | 1.00 | 30.71 | A | C |
| ATOM | 2465 | O | GLN | A | 345 | 32.803 | 45.193 | 65.156 | 1.00 | 30.68 | A | O |
| ATOM | 2466 | N | LEU | A | 346 | 31.403 | 44.163 | 66.596 | 1.00 | 31.46 | A | N |
| ATOM | 2467 | CA | LEU | A | 346 | 30.962 | 43.191 | 65.609 | 1.00 | 32.72 | A | C |
| ATOM | 2468 | CB | LEU | A | 346 | 31.439 | 41.783 | 65.982 | 1.00 | 33.38 | A | C |
| ATOM | 2469 | CG | LEU | A | 346 | 32.915 | 41.359 | 65.905 | 1.00 | 40.75 | A | C |
| ATOM | 2470 | CD1 | LEU | A | 346 | 33.005 | 39.871 | 66.231 | 1.00 | 39.07 | A | C |
| ATOM | 2471 | CD2 | LEU | A | 346 | 33.468 | 41.586 | 64.517 | 1.00 | 38.92 | A | C |
| ATOM | 2472 | C | LEU | A | 346 | 29.442 | 43.148 | 65.476 | 1.00 | 32.53 | A | C |
| ATOM | 2473 | O | LEU | A | 346 | 28.716 | 43.350 | 66.449 | 1.00 | 30.91 | A | O |
| ATOM | 2474 | N | PHE | A | 347 | 28.971 | 42.883 | 64.263 | 1.00 | 30.79 | A | N |
| ATOM | 2475 | CA | PHE | A | 347 | 27.546 | 42.734 | 64.001 | 1.00 | 34.94 | A | C |
| ATOM | 2476 | CB | PHE | A | 347 | 26.828 | 44.088 | 63.898 | 1.00 | 40.29 | A | C |
| ATOM | 2477 | CG | PHE | A | 347 | 27.737 | 45.247 | 63.708 | 1.00 | 45.94 | A | C |
| ATOM | 2478 | CD1 | PHE | A | 347 | 28.102 | 45.661 | 62.437 | 1.00 | 55.10 | A | C |
| ATOM | 2479 | CD2 | PHE | A | 347 | 28.250 | 45.918 | 64.806 | 1.00 | 52.48 | A | C |
| ATOM | 2480 | CE1 | PHE | A | 347 | 28.972 | 46.738 | 62.264 | 1.00 | 57.96 | A | C |
| ATOM | 2481 | CE2 | PHE | A | 347 | 29.115 | 46.988 | 64.650 | 1.00 | 55.14 | A | C |
| ATOM | 2482 | CZ | PHE | A | 347 | 29.479 | 47.397 | 63.378 | 1.00 | 59.41 | A | C |
| ATOM | 2483 | C | PHE | A | 347 | 27.377 | 41.944 | 62.711 | 1.00 | 34.32 | A | C |
| ATOM | 2484 | O | PHE | A | 347 | 27.836 | 42.365 | 61.643 | 1.00 | 34.81 | A | O |
| ATOM | 2485 | N | SER | A | 348 | 26.771 | 40.769 | 62.810 | 1.00 | 32.03 | A | N |
| ATOM | 2486 | CA | SER | A | 348 | 26.569 | 39.986 | 61.612 | 1.00 | 35.97 | A | C |
| ATOM | 2487 | CB | SER | A | 348 | 27.280 | 38.630 | 61.687 | 1.00 | 35.22 | A | C |
| ATOM | 2488 | OG | SER | A | 348 | 26.850 | 37.879 | 62.784 | 1.00 | 35.88 | A | O |
| ATOM | 2489 | C | SER | A | 348 | 25.104 | 39.778 | 61.409 | 1.00 | 33.74 | A | C |
| ATOM | 2490 | O | SER | A | 348 | 24.273 | 40.282 | 62.184 | 1.00 | 30.49 | A | O |
| ATOM | 2491 | N | MET | A | 349 | 24.813 | 39.047 | 60.344 | 1.00 | 35.29 | A | N |
| ATOM | 2492 | CA | MET | A | 349 | 23.459 | 38.698 | 59.917 | 1.00 | 38.30 | A | C |
| ATOM | 2493 | CB | MET | A | 349 | 23.171 | 39.283 | 58.537 | 1.00 | 39.61 | A | C |
| ATOM | 2494 | CG | MET | A | 349 | 23.667 | 40.685 | 58.337 | 1.00 | 42.22 | A | C |
| ATOM | 2495 | SD | MET | A | 349 | 22.453 | 41.837 | 58.917 | 1.00 | 54.09 | A | S |
| ATOM | 2496 | CE | MET | A | 349 | 21.324 | 41.879 | 57.522 | 1.00 | 43.63 | A | C |
| ATOM | 2497 | C | MET | A | 349 | 23.513 | 37.202 | 59.750 | 1.00 | 35.81 | A | C |
| ATOM | 2498 | O | MET | A | 349 | 24.043 | 36.730 | 58.747 | 1.00 | 34.73 | A | O |
| ATOM | 2499 | N | GLU | A | 350 | 22.975 | 36.443 | 60.691 | 1.00 | 34.66 | A | N |
| ATOM | 2500 | CA | GLU | A | 350 | 23.051 | 34.998 | 60.532 | 1.00 | 35.99 | A | C |
| ATOM | 2501 | CB | GLU | A | 350 | 22.260 | 34.287 | 61.622 | 1.00 | 36.59 | A | C |
| ATOM | 2502 | CG | GLU | A | 350 | 22.686 | 32.839 | 61.823 | 1.00 | 37.08 | A | C |
| ATOM | 2503 | CD | GLU | A | 350 | 21.657 | 32.038 | 62.605 | 1.00 | 37.90 | A | C |
| ATOM | 2504 | OE1 | GLU | A | 350 | 21.973 | 30.911 | 63.064 | 1.00 | 38.96 | A | O |
| ATOM | 2505 | OE2 | GLU | A | 350 | 20.521 | 32.539 | 62.745 | 1.00 | 33.59 | A | O |
| ATOM | 2506 | C | GLU | A | 350 | 22.498 | 34.622 | 59.159 | 1.00 | 33.37 | A | C |
| ATOM | 2507 | O | GLU | A | 350 | 21.429 | 35.069 | 58.783 | 1.00 | 33.19 | A | O |
| ATOM | 2508 | N | GLY | A | 351 | 23.237 | 33.817 | 58.407 | 1.00 | 33.13 | A | N |
| ATOM | 2509 | CA | GLY | A | 351 | 22.765 | 33.411 | 57.092 | 1.00 | 35.91 | A | C |
| ATOM | 2510 | C | GLY | A | 351 | 23.332 | 34.232 | 55.949 | 1.00 | 37.63 | A | C |
| ATOM | 2511 | O | GLY | A | 351 | 23.081 | 33.933 | 54.776 | 1.00 | 38.23 | A | O |
| ATOM | 2512 | N | PHE | A | 352 | 24.114 | 35.257 | 56.278 | 1.00 | 36.90 | A | N |
| ATOM | 2513 | CA | PHE | A | 352 | 24.688 | 36.115 | 55.247 | 1.00 | 37.44 | A | C |
| ATOM | 2514 | CB | PHE | A | 352 | 23.895 | 37.415 | 55.143 | 1.00 | 37.21 | A | C |
| ATOM | 2515 | CG | PHE | A | 352 | 24.426 | 38.367 | 54.107 | 1.00 | 36.39 | A | C |
| ATOM | 2516 | CD1 | PHE | A | 352 | 24.204 | 38.141 | 52.754 | 1.00 | 39.20 | A | C |
| ATOM | 2517 | CD2 | PHE | A | 352 | 25.197 | 39.454 | 54.478 | 1.00 | 39.46 | A | C |
| ATOM | 2518 | CE1 | PHE | A | 352 | 24.750 | 38.986 | 51.783 | 1.00 | 36.34 | A | C |
| ATOM | 2519 | CE2 | PHE | A | 352 | 25.745 | 40.302 | 53.514 | 1.00 | 40.21 | A | C |
| ATOM | 2520 | CZ | PHE | A | 352 | 25.520 | 40.062 | 52.166 | 1.00 | 36.50 | A | C |
| ATOM | 2521 | C | PHE | A | 352 | 26.155 | 36.453 | 55.473 | 1.00 | 38.95 | A | C |
| ATOM | 2522 | O | PHE | A | 352 | 27.034 | 35.980 | 54.739 | 1.00 | 40.41 | A | O |
| ATOM | 2523 | N | GLY | A | 353 | 26.431 | 37.272 | 56.484 | 1.00 | 37.81 | A | N |
| ATOM | 2524 | CA | GLY | A | 353 | 27.810 | 37.657 | 56.729 | 1.00 | 37.69 | A | C |
| ATOM | 2525 | C | GLY | A | 353 | 28.010 | 38.520 | 57.960 | 1.00 | 37.66 | A | C |
| ATOM | 2526 | O | GLY | A | 353 | 27.083 | 38.742 | 58.732 | 1.00 | 39.65 | A | O |
| ATOM | 2527 | N | THR | A | 354 | 29.220 | 39.041 | 58.131 | 1.00 | 36.84 | A | N |
| ATOM | 2528 | CA | THR | A | 354 | 29.542 | 39.830 | 59.305 | 1.00 | 34.39 | A | C |
| ATOM | 2529 | CB | THR | A | 354 | 30.398 | 38.996 | 60.275 | 1.00 | 31.81 | A | C |
| ATOM | 2530 | OG1 | THR | A | 354 | 29.711 | 37.781 | 60.578 | 1.00 | 29.22 | A | O |
| ATOM | 2531 | CG2 | THR | A | 354 | 30.657 | 39.750 | 61.585 | 1.00 | 30.05 | A | C |
| ATOM | 2532 | C | THR | A | 354 | 30.302 | 41.109 | 59.019 | 1.00 | 34.65 | A | C |
| ATOM | 2533 | O | THR | A | 354 | 31.152 | 41.148 | 58.128 | 1.00 | 37.11 | A | O |
| ATOM | 2534 | N | ASP | A | 355 | 29.985 | 42.156 | 59.775 | 1.00 | 32.31 | A | N |
| ATOM | 2535 | CA | ASP | A | 355 | 30.703 | 43.423 | 59.669 | 1.00 | 34.66 | A | C |
| ATOM | 2536 | CB | ASP | A | 355 | 29.742 | 44.601 | 59.488 | 1.00 | 34.29 | A | C |
| ATOM | 2537 | CG | ASP | A | 355 | 29.205 | 44.699 | 58.059 | 1.00 | 37.14 | A | C |
| ATOM | 2538 | OD1 | ASP | A | 355 | 29.333 | 43.714 | 57.307 | 1.00 | 37.72 | A | O |
| ATOM | 2539 | OD2 | ASP | A | 355 | 28.651 | 45.753 | 57.690 | 1.00 | 38.60 | A | O |
| ATOM | 2540 | C | ASP | A | 355 | 31.485 | 43.576 | 60.977 | 1.00 | 34.64 | A | C |
| ATOM | 2541 | O | ASP | A | 355 | 30.969 | 43.308 | 62.065 | 1.00 | 36.90 | A | O |
| ATOM | 2542 | N | ALA | A | 356 | 32.751 | 43.939 | 60.852 | 1.00 | 33.90 | A | N |
| ATOM | 2543 | CA | ALA | A | 356 | 33.613 | 44.151 | 62.001 | 1.00 | 33.47 | A | C |
| ATOM | 2544 | CB | ALA | A | 356 | 34.774 | 43.172 | 61.981 | 1.00 | 33.50 | A | C |
| ATOM | 2545 | C | ALA | A | 356 | 34.109 | 45.565 | 61.785 | 1.00 | 33.63 | A | C |
| ATOM | 2546 | O | ALA | A | 356 | 34.622 | 45.891 | 60.710 | 1.00 | 33.57 | A | O |
| ATOM | 2547 | N | VAL | A | 357 | 33.938 | 46.412 | 62.790 | 1.00 | 32.07 | A | N |
| ATOM | 2548 | CA | VAL | A | 357 | 34.357 | 47.791 | 62.664 | 1.00 | 31.18 | A | C |
| ATOM | 2549 | CB | VAL | A | 357 | 33.127 | 48.721 | 62.718 | 1.00 | 33.27 | A | C |
| ATOM | 2550 | CG1 | VAL | A | 357 | 33.550 | 50.169 | 62.554 | 1.00 | 31.15 | A | C |
| ATOM | 2551 | CG2 | VAL | A | 357 | 32.130 | 48.320 | 61.622 | 1.00 | 37.40 | A | C |
| ATOM | 2552 | C | VAL | A | 357 | 35.350 | 48.223 | 63.730 | 1.00 | 32.58 | A | C |
| ATOM | 2553 | O | VAL | A | 357 | 35.135 | 48.006 | 64.932 | 1.00 | 35.85 | A | O |
| ATOM | 2554 | N | ILE | A | 358 | 36.440 | 48.833 | 63.286 | 1.00 | 31.65 | A | N |
| ATOM | 2555 | CA | ILE | A | 358 | 37.443 | 49.359 | 64.191 | 1.00 | 31.19 | A | C |
| ATOM | 2556 | CB | ILE | A | 358 | 38.881 | 49.033 | 63.719 | 1.00 | 31.87 | A | C |
| ATOM | 2557 | CG2 | ILE | A | 358 | 39.889 | 49.658 | 64.683 | 1.00 | 32.09 | A | C |
| ATOM | 2558 | CG1 | ILE | A | 358 | 39.095 | 47.521 | 63.638 | 1.00 | 28.66 | A | C |
| ATOM | 2559 | CD1 | ILE | A | 358 | 40.427 | 47.181 | 63.020 | 1.00 | 34.35 | A | C |
| ATOM | 2560 | C | ILE | A | 358 | 37.289 | 50.875 | 64.183 | 1.00 | 29.95 | A | C |
| ATOM | 2561 | O | ILE | A | 358 | 37.400 | 51.503 | 63.132 | 1.00 | 30.55 | A | O |
| ATOM | 2562 | N | TYR | A | 359 | 37.014 | 51.448 | 65.348 | 1.00 | 30.07 | A | N |
| ATOM | 2563 | CA | TYR | A | 359 | 36.871 | 52.889 | 65.513 | 1.00 | 29.53 | A | C |
| ATOM | 2564 | CB | TYR | A | 359 | 35.683 | 53.228 | 66.408 | 1.00 | 28.24 | A | C |
| ATOM | 2565 | CG | TYR | A | 359 | 34.350 | 52.848 | 65.830 | 1.00 | 28.67 | A | C |
| ATOM | 2566 | CD1 | TYR | A | 359 | 33.669 | 51.722 | 66.287 | 1.00 | 25.45 | A | C |
| ATOM | 2567 | CE1 | TYR | A | 359 | 32.442 | 51.349 | 65.728 | 1.00 | 30.75 | A | C |
| ATOM | 2568 | CD2 | TYR | A | 359 | 33.782 | 53.603 | 64.793 | 1.00 | 28.60 | A | C |
| ATOM | 2569 | CE2 | TYR | A | 359 | 32.568 | 53.237 | 64.221 | 1.00 | 28.61 | A | C |
| ATOM | 2570 | CZ | TYR | A | 359 | 31.904 | 52.114 | 64.697 | 1.00 | 29.92 | A | C |
| ATOM | 2571 | OH | TYR | A | 359 | 30.684 | 51.771 | 64.160 | 1.00 | 37.02 | A | O |
| ATOM | 2572 | C | TYR | A | 359 | 38.107 | 53.438 | 66.192 | 1.00 | 29.72 | A | C |
| ATOM | 2573 | O | TYR | A | 359 | 38.426 | 53.027 | 67.308 | 1.00 | 33.61 | A | O |
| ATOM | 2574 | N | LEU | A | 360 | 38.793 | 54.360 | 65.521 | 1.00 | 31.41 | A | N |
| ATOM | 2575 | CA | LEU | A | 360 | 39.990 | 55.012 | 66.050 | 1.00 | 30.20 | A | C |
| ATOM | 2576 | CB | LEU | A | 360 | 41.190 | 54.780 | 65.137 | 1.00 | 26.31 | A | C |
| ATOM | 2577 | CG | LEU | A | 360 | 41.534 | 53.352 | 64.707 | 1.00 | 27.26 | A | C |
| ATOM | 2578 | CD1 | LEU | A | 360 | 42.612 | 53.427 | 63.613 | 1.00 | 24.51 | A | C |
| ATOM | 2579 | CD2 | LEU | A | 360 | 42.001 | 52.505 | 65.947 | 1.00 | 23.61 | A | C |
| ATOM | 2580 | C | LEU | A | 360 | 39.698 | 56.511 | 66.077 | 1.00 | 34.34 | A | C |
| ATOM | 2581 | O | LEU | A | 360 | 38.793 | 57.000 | 65.367 | 1.00 | 32.13 | A | O |
| ATOM | 2582 | N | LYS | A | 361 | 40.451 | 57.251 | 66.885 | 1.00 | 33.61 | A | N |
| ATOM | 2583 | CA | LYS | A | 361 | 40.251 | 58.687 | 66.927 | 1.00 | 32.70 | A | C |
| ATOM | 2584 | CB | LYS | A | 361 | 40.813 | 59.283 | 68.200 | 1.00 | 33.98 | A | C |
| ATOM | 2585 | CG | LYS | A | 361 | 40.040 | 58.928 | 69.420 | 1.00 | 36.54 | A | C |
| ATOM | 2586 | CD | LYS | A | 361 | 38.624 | 59.480 | 69.384 | 1.00 | 36.01 | A | C |
| ATOM | 2587 | CE | LYS | A | 361 | 38.054 | 59.427 | 70.790 | 1.00 | 36.08 | A | C |
| ATOM | 2588 | NZ | LYS | A | 361 | 36.591 | 59.559 | 70.829 | 1.00 | 37.98 | A | N |
| ATOM | 2589 | C | LYS | A | 361 | 41.034 | 59.214 | 65.759 | 1.00 | 34.69 | A | C |
| ATOM | 2590 | O | LYS | A | 361 | 42.168 | 58.774 | 65.542 | 1.00 | 35.70 | A | O |
| ATOM | 2591 | N | ALA | A | 362 | 40.448 | 60.139 | 65.003 | 1.00 | 34.32 | A | N |
| ATOM | 2592 | CA | ALA | A | 362 | 41.133 | 60.724 | 63.852 | 1.00 | 36.74 | A | C |
| ATOM | 2593 | CB | ALA | A | 362 | 40.118 | 61.477 | 62.959 | 1.00 | 35.46 | A | C |
| ATOM | 2594 | C | ALA | A | 362 | 42.256 | 61.680 | 64.271 | 1.00 | 37.21 | A | C |
| ATOM | 2595 | O | ALA | A | 362 | 43.279 | 61.768 | 63.597 | 1.00 | 38.21 | A | O |
| ATOM | 2596 | N | LEU | A | 363 | 42.065 | 62.395 | 65.379 | 1.00 | 38.49 | A | N |
| ATOM | 2597 | CA | LEU | A | 363 | 43.063 | 63.367 | 65.838 | 1.00 | 41.42 | A | C |
| ATOM | 2598 | CB | LEU | A | 363 | 42.356 | 64.619 | 66.371 | 1.00 | 40.19 | A | C |
| ATOM | 2599 | CG | LEU | A | 363 | 41.310 | 65.301 | 65.474 | 1.00 | 48.26 | A | C |
| ATOM | 2600 | CD1 | LEU | A | 363 | 40.695 | 66.489 | 66.227 | 1.00 | 43.70 | A | C |
| ATOM | 2601 | CD2 | LEU | A | 363 | 41.942 | 65.756 | 64.160 | 1.00 | 46.82 | A | C |
| ATOM | 2602 | C | LEU | A | 363 | 44.027 | 62.842 | 66.914 | 1.00 | 39.75 | A | C |
| ATOM | 2603 | O | LEU | A | 363 | 43.608 | 62.206 | 67.887 | 1.00 | 40.08 | A | O |
| ATOM | 2604 | N | SER | A | 364 | 45.313 | 63.125 | 66.745 | 1.00 | 39.67 | A | N |
| ATOM | 2605 | CA | SER | A | 364 | 46.320 | 62.696 | 67.710 | 1.00 | 44.28 | A | C |
| ATOM | 2606 | CB | SER | A | 364 | 47.715 | 63.107 | 67.244 | 1.00 | 40.72 | A | C |
| ATOM | 2607 | OG | SER | A | 364 | 47.821 | 64.514 | 67.197 | 1.00 | 46.00 | A | O |
| ATOM | 2608 | C | SER | A | 364 | 46.008 | 63.341 | 69.058 | 1.00 | 46.09 | A | C |
| ATOM | 2609 | O | SER | A | 364 | 46.379 | 62.831 | 70.106 | 1.00 | 50.30 | A | O |
| ATOM | 2610 | N | THR | A | 365 | 45.311 | 64.465 | 69.026 | 1.00 | 47.47 | A | N |
| ATOM | 2611 | CA | THR | A | 365 | 44.915 | 65.155 | 70.241 | 1.00 | 47.55 | A | C |
| ATOM | 2612 | CB | THR | A | 365 | 44.202 | 66.462 | 69.897 | 1.00 | 48.32 | A | C |
| ATOM | 2613 | OG1 | THR | A | 365 | 45.061 | 67.275 | 69.096 | 1.00 | 56.22 | A | O |
| ATOM | 2614 | CG2 | THR | A | 365 | 43.833 | 67.206 | 71.152 | 1.00 | 52.51 | A | C |
| ATOM | 2615 | C | THR | A | 365 | 43.933 | 64.324 | 71.071 | 1.00 | 45.20 | A | C |
| ATOM | 2616 | O | THR | A | 365 | 43.938 | 64.385 | 72.303 | 1.00 | 44.22 | A | O |
| ATOM | 2617 | N | ASP | A | 366 | 43.067 | 63.574 | 70.392 | 1.00 | 45.27 | A | N |
| ATOM | 2618 | CA | ASP | A | 366 | 42.045 | 62.776 | 71.080 | 1.00 | 45.49 | A | C |
| ATOM | 2619 | CB | ASP | A | 366 | 40.792 | 62.623 | 70.193 | 1.00 | 47.74 | A | C |
| ATOM | 2620 | CG | ASP | A | 366 | 40.189 | 63.967 | 69.758 | 1.00 | 49.27 | A | C |
| ATOM | 2621 | OD1 | ASP | A | 366 | 39.863 | 64.792 | 70.630 | 1.00 | 50.58 | A | O |
| ATOM | 2622 | OD2 | ASP | A | 366 | 40.024 | 64.196 | 68.541 | 1.00 | 51.70 | A | O |
| ATOM | 2623 | C | ASP | A | 366 | 42.539 | 61.374 | 71.463 | 1.00 | 42.93 | A | C |
| ATOM | 2624 | O | ASP | A | 366 | 41.888 | 60.668 | 72.231 | 1.00 | 39.42 | A | O |
| ATOM | 2625 | N | SER | A | 367 | 43.685 | 60.985 | 70.916 | 1.00 | 39.98 | A | N |
| ATOM | 2626 | CA | SER | A | 367 | 44.253 | 59.670 | 71.154 | 1.00 | 43.17 | A | C |
| ATOM | 2627 | CB | SER | A | 367 | 45.169 | 59.301 | 69.990 | 1.00 | 42.52 | A | C |
| ATOM | 2628 | OG | SER | A | 367 | 44.473 | 59.419 | 68.759 | 1.00 | 44.52 | A | O |
| ATOM | 2629 | C | SER | A | 367 | 45.026 | 59.630 | 72.468 | 1.00 | 43.54 | A | C |
| ATOM | 2630 | O | SER | A | 367 | 46.263 | 59.671 | 72.485 | 1.00 | 42.97 | A | O |
| ATOM | 2631 | N | VAL | A | 368 | 44.281 | 59.553 | 73.565 | 1.00 | 39.90 | A | N |
| ATOM | 2632 | CA | VAL | A | 368 | 44.878 | 59.519 | 74.884 | 1.00 | 42.57 | A | C |
| ATOM | 2633 | CB | VAL | A | 368 | 44.195 | 60.533 | 75.841 | 1.00 | 44.72 | A | C |
| ATOM | 2634 | CG1 | VAL | A | 368 | 44.876 | 60.481 | 77.213 | 1.00 | 45.90 | A | C |
| ATOM | 2635 | CG2 | VAL | A | 368 | 44.265 | 61.947 | 75.256 | 1.00 | 44.42 | A | C |
| ATOM | 2636 | C | VAL | A | 368 | 44.779 | 58.128 | 75.501 | 1.00 | 42.95 | A | C |
| ATOM | 2637 | O | VAL | A | 368 | 43.713 | 57.494 | 75.491 | 1.00 | 43.52 | A | O |
| ATOM | 2638 | N | GLU | A | 369 | 45.902 | 57.658 | 76.027 | 1.00 | 42.51 | A | N |
| ATOM | 2639 | CA | GLU | A | 369 | 45.969 | 56.350 | 76.664 | 1.00 | 42.56 | A | C |
| ATOM | 2640 | CB | GLU | A | 369 | 47.230 | 56.228 | 77.514 | 1.00 | 41.33 | A | C |
| ATOM | 2641 | CG | GLU | A | 369 | 48.536 | 56.060 | 76.786 | 1.00 | 41.65 | A | C |
| ATOM | 2642 | CD | GLU | A | 369 | 49.660 | 55.729 | 77.764 | 1.00 | 47.28 | A | C |
| ATOM | 2643 | OE1 | GLU | A | 369 | 49.994 | 56.603 | 78.609 | 1.00 | 43.06 | A | O |
| ATOM | 2644 | OE2 | GLU | A | 369 | 50.194 | 54.591 | 77.695 | 1.00 | 43.17 | A | O |
| ATOM | 2645 | C | GLU | A | 369 | 44.785 | 56.136 | 77.585 | 1.00 | 42.51 | A | C |
| ATOM | 2646 | O | GLU | A | 369 | 44.471 | 56.999 | 78.398 | 1.00 | 42.34 | A | O |
| ATOM | 2647 | N | ARG | A | 370 | 44.125 | 54.996 | 77.445 | 1.00 | 41.76 | A | N |
| ATOM | 2648 | CA | ARG | A | 370 | 43.013 | 54.647 | 78.310 | 1.00 | 47.45 | A | C |
| ATOM | 2649 | CB | ARG | A | 370 | 41.889 | 53.990 | 77.511 | 1.00 | 48.46 | A | C |
| ATOM | 2650 | CG | ARG | A | 370 | 40.867 | 53.318 | 78.386 | 1.00 | 54.82 | A | C |
| ATOM | 2651 | CD | ARG | A | 370 | 39.475 | 53.404 | 77.810 | 1.00 | 62.90 | A | C |
| ATOM | 2652 | NE | ARG | A | 370 | 38.542 | 52.639 | 78.630 | 1.00 | 73.26 | A | N |
| ATOM | 2653 | CZ | ARG | A | 370 | 37.221 | 52.722 | 78.541 | 1.00 | 78.05 | A | C |
| ATOM | 2654 | NH1 | ARG | A | 370 | 36.669 | 53.546 | 77.663 | 1.00 | 81.18 | A | N |
| ATOM | 2655 | NH2 | ARG | A | 370 | 36.455 | 51.974 | 79.328 | 1.00 | 80.40 | A | N |
| ATOM | 2656 | C | ARG | A | 370 | 43.617 | 53.661 | 79.314 | 1.00 | 51.12 | A | C |
| ATOM | 2657 | O | ARG | A | 370 | 43.910 | 52.508 | 78.981 | 1.00 | 49.32 | A | O |
| ATOM | 2658 | N | LEU | A | 371 | 43.818 | 54.136 | 80.538 | 1.00 | 56.78 | A | N |
| ATOM | 2659 | CA | LEU | A | 371 | 44.441 | 53.351 | 81.598 | 1.00 | 61.41 | A | C |
| ATOM | 2660 | CB | LEU | A | 371 | 45.592 | 54.149 | 82.211 | 1.00 | 58.88 | A | C |
| ATOM | 2661 | CG | LEU | A | 371 | 46.939 | 54.267 | 81.513 | 1.00 | 58.00 | A | C |
| ATOM | 2662 | CD1 | LEU | A | 371 | 46.747 | 54.524 | 80.062 | 1.00 | 62.16 | A | C |
| ATOM | 2663 | CD2 | LEU | A | 371 | 47.732 | 55.394 | 82.154 | 1.00 | 61.67 | A | C |
| ATOM | 2664 | C | LEU | A | 371 | 43.520 | 52.934 | 82.729 | 1.00 | 66.96 | A | C |
| ATOM | 2665 | O | LEU | A | 371 | 42.737 | 53.731 | 83.230 | 1.00 | 67.38 | A | O |
| ATOM | 2666 | N | PRO | A | 372 | 43.612 | 51.669 | 83.154 | 1.00 | 73.57 | A | N |
| ATOM | 2667 | CD | PRO | A | 372 | 44.362 | 50.551 | 82.551 | 1.00 | 73.78 | A | C |
| ATOM | 2668 | CA | PRO | A | 372 | 42.767 | 51.206 | 84.253 | 1.00 | 80.01 | A | C |
| ATOM | 2669 | CB | PRO | A | 372 | 42.724 | 49.703 | 84.028 | 1.00 | 77.65 | A | C |
| ATOM | 2670 | CG | PRO | A | 372 | 44.117 | 49.418 | 83.540 | 1.00 | 76.40 | A | C |
| ATOM | 2671 | C | PRO | A | 372 | 43.500 | 51.588 | 85.545 | 1.00 | 87.03 | A | C |
| ATOM | 2672 | O | PRO | A | 372 | 44.722 | 51.470 | 85.615 | 1.00 | 86.14 | A | O |
| ATOM | 2673 | N | VAL | A | 373 | 42.769 | 52.047 | 86.556 | 1.00 | 97.04 | A | N |
| ATOM | 2674 | CA | VAL | A | 373 | 43.399 | 52.442 | 87.816 | 1.00 | 108.10 | A | C |
| ATOM | 2675 | CB | VAL | A | 373 | 43.335 | 53.977 | 88.011 | 1.00 | 107.81 | A | C |
| ATOM | 2676 | CG1 | VAL | A | 373 | 43.882 | 54.362 | 89.379 | 1.00 | 108.28 | A | C |
| ATOM | 2677 | CG2 | VAL | A | 373 | 44.137 | 54.665 | 86.922 | 1.00 | 108.19 | A | C |
| ATOM | 2678 | C | VAL | A | 373 | 42.777 | 51.773 | 89.038 | 1.00 | 116.26 | A | C |
| ATOM | 2679 | O | VAL | A | 373 | 41.598 | 51.416 | 89.035 | 1.00 | 116.09 | A | O |
| ATOM | 2680 | N | TYR | A | 374 | 43.584 | 51.611 | 90.083 | 1.00 | 127.09 | A | N |
| ATOM | 2681 | CA | TYR | A | 374 | 43.128 | 50.999 | 91.323 | 1.00 | 137.70 | A | C |
| ATOM | 2682 | CB | TYR | A | 374 | 44.282 | 50.269 | 92.014 | 1.00 | 142.41 | A | C |
| ATOM | 2683 | CG | TYR | A | 374 | 43.905 | 49.743 | 93.376 | 1.00 | 148.20 | A | C |
| ATOM | 2684 | CD1 | TYR | A | 374 | 42.841 | 48.854 | 93.524 | 1.00 | 151.00 | A | C |
| ATOM | 2685 | CE1 | TYR | A | 374 | 42.456 | 48.395 | 94.776 | 1.00 | 154.04 | A | C |
| ATOM | 2686 | CD2 | TYR | A | 374 | 44.583 | 50.160 | 94.521 | 1.00 | 150.58 | A | C |
| ATOM | 2687 | CE2 | TYR | A | 374 | 44.206 | 49.707 | 95.782 | 1.00 | 153.76 | A | C |
| ATOM | 2688 | CZ | TYR | A | 374 | 43.140 | 48.824 | 95.901 | 1.00 | 154.79 | A | C |
| ATOM | 2689 | OH | TYR | A | 374 | 42.749 | 48.374 | 97.142 | 1.00 | 156.36 | A | O |
| ATOM | 2690 | C | TYR | A | 374 | 42.540 | 52.031 | 92.286 | 1.00 | 142.02 | A | C |
| ATOM | 2691 | O | TYR | A | 374 | 43.249 | 52.908 | 92.781 | 1.00 | 142.58 | A | O |
| ATOM | 2692 | N | ASN | A | 375 | 41.242 | 51.916 | 92.547 | 1.00 | 146.93 | A | N |
| ATOM | 2693 | CA | ASN | A | 375 | 40.548 | 52.821 | 93.458 | 1.00 | 151.95 | A | C |
| ATOM | 2694 | CB | ASN | A | 375 | 39.835 | 53.933 | 92.682 | 1.00 | 153.07 | A | C |
| ATOM | 2695 | CG | ASN | A | 375 | 40.788 | 54.811 | 91.905 | 1.00 | 154.56 | A | C |
| ATOM | 2696 | OD1 | ASN | A | 375 | 41.681 | 55.437 | 92.476 | 1.00 | 155.59 | A | O |
| ATOM | 2697 | ND2 | ASN | A | 375 | 40.598 | 54.868 | 90.592 | 1.00 | 155.33 | A | N |
| ATOM | 2698 | C | ASN | A | 375 | 39.508 | 52.034 | 94.240 | 1.00 | 154.74 | A | C |
| ATOM | 2699 | O | ASN | A | 375 | 39.415 | 50.814 | 94.109 | 1.00 | 155.32 | A | O |
| ATOM | 2700 | N | LYS | A | 376 | 38.733 | 52.734 | 95.062 | 1.00 | 157.93 | A | N |
| ATOM | 2701 | CA | LYS | A | 376 | 37.670 | 52.096 | 95.830 | 1.00 | 160.85 | A | C |
| ATOM | 2702 | CB | LYS | A | 376 | 37.104 | 53.065 | 96.877 | 1.00 | 158.72 | A | C |
| ATOM | 2703 | CG | LYS | A | 376 | 35.693 | 52.728 | 97.355 | 1.00 | 155.62 | A | C |
| ATOM | 2704 | CD | LYS | A | 376 | 35.621 | 51.351 | 97.997 | 1.00 | 153.03 | A | C |
| ATOM | 2705 | CE | LYS | A | 376 | 34.177 | 50.913 | 98.201 | 1.00 | 151.25 | A | C |
| ATOM | 2706 | NZ | LYS | A | 376 | 33.408 | 51.885 | 99.023 | 1.00 | 149.56 | A | N |
| ATOM | 2707 | C | LYS | A | 376 | 36.592 | 51.728 | 94.820 | 1.00 | 164.21 | A | C |
| ATOM | 2708 | O | LYS | A | 376 | 35.739 | 50.877 | 95.072 | 1.00 | 164.75 | A | O |
| ATOM | 2709 | N | SER | A | 377 | 36.652 | 52.388 | 93.667 | 1.00 | 167.90 | A | N |
| ATOM | 2710 | CA | SER | A | 377 | 35.707 | 52.162 | 92.581 | 1.00 | 171.27 | A | C |
| ATOM | 2711 | CB | SER | A | 377 | 35.854 | 53.260 | 91.522 | 1.00 | 171.66 | A | C |
| ATOM | 2712 | OG | SER | A | 377 | 35.018 | 53.009 | 90.405 | 1.00 | 172.64 | A | O |
| ATOM | 2713 | C | SER | A | 377 | 35.938 | 50.797 | 91.942 | 1.00 | 173.11 | A | C |
| ATOM | 2714 | O | SER | A | 377 | 35.004 | 50.014 | 91.774 | 1.00 | 173.50 | A | O |
| ATOM | 2715 | N | ALA | A | 378 | 37.190 | 50.519 | 91.592 | 1.00 | 175.00 | A | N |
| ATOM | 2716 | CA | ALA | A | 378 | 37.558 | 49.252 | 90.969 | 1.00 | 176.65 | A | C |
| ATOM | 2717 | CB | ALA | A | 378 | 38.998 | 49.318 | 90.474 | 1.00 | 176.44 | A | C |
| ATOM | 2718 | C | ALA | A | 378 | 37.391 | 48.076 | 91.928 | 1.00 | 177.84 | A | C |
| ATOM | 2719 | O | ALA | A | 378 | 37.224 | 46.935 | 91.500 | 1.00 | 178.16 | A | O |
| ATOM | 2720 | N | TRP | A | 379 | 37.428 | 48.364 | 93.225 | 1.00 | 179.12 | A | N |
| ATOM | 2721 | CA | TRP | A | 379 | 37.292 | 47.335 | 94.253 | 1.00 | 180.06 | A | C |
| ATOM | 2722 | CB | TRP | A | 379 | 37.541 | 47.939 | 95.640 | 1.00 | 183.95 | A | C |
| ATOM | 2723 | CG | TRP | A | 379 | 37.505 | 46.922 | 96.744 | 1.00 | 188.69 | A | C |
| ATOM | 2724 | CD2 | TRP | A | 379 | 36.373 | 46.575 | 97.552 | 1.00 | 191.18 | A | C |
| ATOM | 2725 | CE2 | TRP | A | 379 | 36.783 | 45.543 | 98.425 | 1.00 | 192.04 | A | C |
| ATOM | 2726 | CE3 | TRP | A | 379 | 35.050 | 47.035 | 97.621 | 1.00 | 192.49 | A | C |
| ATOM | 2727 | CD1 | TRP | A | 379 | 38.527 | 46.110 | 97.148 | 1.00 | 189.99 | A | C |
| ATOM | 2728 | NE1 | TRP | A | 379 | 38.101 | 45.279 | 98.158 | 1.00 | 191.51 | A | N |
| ATOM | 2729 | CZ2 | TRP | A | 379 | 35.915 | 44.961 | 99.360 | 1.00 | 192.95 | A | C |
| ATOM | 2730 | CZ3 | TRP | A | 379 | 34.187 | 46.458 | 98.550 | 1.00 | 193.35 | A | C |
| ATOM | 2731 | CH2 | TRP | A | 379 | 34.626 | 45.430 | 99.407 | 1.00 | 193.53 | A | C |
| ATOM | 2732 | C | TRP | A | 379 | 35.929 | 46.636 | 94.254 | 1.00 | 178.45 | A | C |
| ATOM | 2733 | O | TRP | A | 379 | 35.858 | 45.407 | 94.292 | 1.00 | 178.31 | A | O |
| ATOM | 2734 | N | ARG | A | 380 | 34.854 | 47.418 | 94.209 | 1.00 | 176.25 | A | N |
| ATOM | 2735 | CA | ARG | A | 380 | 33.502 | 46.867 | 94.242 | 1.00 | 174.14 | A | C |
| ATOM | 2736 | CB | ARG | A | 380 | 32.515 | 47.950 | 94.694 | 1.00 | 171.20 | A | C |
| ATOM | 2737 | CG | ARG | A | 380 | 32.694 | 49.294 | 94.016 | 1.00 | 167.46 | A | C |
| ATOM | 2738 | CD | ARG | A | 380 | 31.978 | 50.390 | 94.789 | 1.00 | 163.97 | A | C |
| ATOM | 2739 | NE | ARG | A | 380 | 32.202 | 51.711 | 94.209 | 1.00 | 160.73 | A | N |
| ATOM | 2740 | CZ | ARG | A | 380 | 31.833 | 52.851 | 94.782 | 1.00 | 159.10 | A | C |
| ATOM | 2741 | NH1 | ARG | A | 380 | 31.218 | 52.838 | 95.957 | 1.00 | 158.01 | A | N |
| ATOM | 2742 | NH2 | ARG | A | 380 | 32.078 | 54.006 | 94.180 | 1.00 | 158.33 | A | N |
| ATOM | 2743 | C | ARG | A | 380 | 32.984 | 46.179 | 92.977 | 1.00 | 174.55 | A | C |
| ATOM | 2744 | O | ARG | A | 380 | 31.841 | 45.722 | 92.946 | 1.00 | 174.27 | A | O |
| ATOM | 2745 | N | HIS | A | 381 | 33.812 | 46.101 | 91.938 | 1.00 | 175.28 | A | N |
| ATOM | 2746 | CA | HIS | A | 381 | 33.405 | 45.424 | 90.707 | 1.00 | 175.95 | A | C |
| ATOM | 2747 | CB | HIS | A | 381 | 34.195 | 45.941 | 89.499 | 1.00 | 177.09 | A | C |
| ATOM | 2748 | CG | HIS | A | 381 | 33.743 | 47.280 | 88.999 | 1.00 | 178.38 | A | C |
| ATOM | 2749 | CD2 | HIS | A | 381 | 33.192 | 47.651 | 87.818 | 1.00 | 178.85 | A | C |
| ATOM | 2750 | ND1 | HIS | A | 381 | 33.851 | 48.432 | 89.749 | 1.00 | 179.01 | A | N |
| ATOM | 2751 | CE1 | HIS | A | 381 | 33.388 | 49.455 | 89.051 | 1.00 | 179.30 | A | C |
| ATOM | 2752 | NE2 | HIS | A | 381 | 32.982 | 49.008 | 87.876 | 1.00 | 179.20 | A | N |
| ATOM | 2753 | C | HIS | A | 381 | 33.674 | 43.933 | 90.886 | 1.00 | 175.62 | A | C |
| ATOM | 2754 | O | HIS | A | 381 | 33.248 | 43.107 | 90.078 | 1.00 | 175.63 | A | O |
| ATOM | 2755 | N | TYR | A | 382 | 34.389 | 43.607 | 91.961 | 1.00 | 175.03 | A | N |
| ATOM | 2756 | CA | TYR | A | 382 | 34.739 | 42.230 | 92.293 | 1.00 | 174.36 | A | C |
| ATOM | 2757 | CB | TYR | A | 382 | 36.203 | 42.144 | 92.729 | 1.00 | 174.51 | A | C |
| ATOM | 2758 | CG | TYR | A | 382 | 37.197 | 42.458 | 91.635 | 1.00 | 174.84 | A | C |
| ATOM | 2759 | CD1 | TYR | A | 382 | 37.353 | 41.607 | 90.542 | 1.00 | 174.73 | A | C |
| ATOM | 2760 | CE1 | TYR | A | 382 | 38.272 | 41.892 | 89.534 | 1.00 | 174.59 | A | C |
| ATOM | 2761 | CD2 | TYR | A | 382 | 37.985 | 43.606 | 91.695 | 1.00 | 175.00 | A | C |
| ATOM | 2762 | CE2 | TYR | A | 382 | 38.905 | 43.900 | 90.695 | 1.00 | 174.89 | A | C |
| ATOM | 2763 | CZ | TYR | A | 382 | 39.043 | 43.039 | 89.618 | 1.00 | 174.65 | A | C |
| ATOM | 2764 | OH | TYR | A | 382 | 39.950 | 43.329 | 88.626 | 1.00 | 174.41 | A | O |
| ATOM | 2765 | C | TYR | A | 382 | 33.856 | 41.707 | 93.419 | 1.00 | 173.79 | A | C |
| ATOM | 2766 | O | TYR | A | 382 | 34.080 | 40.612 | 93.936 | 1.00 | 173.79 | A | O |
| ATOM | 2767 | N | GLN | A | 383 | 32.858 | 42.497 | 93.802 | 1.00 | 173.06 | A | N |
| ATOM | 2768 | CA | GLN | A | 383 | 31.946 | 42.103 | 94.870 | 1.00 | 171.92 | A | C |
| ATOM | 2769 | CB | GLN | A | 383 | 31.640 | 43.296 | 95.784 | 1.00 | 172.68 | A | C |
| ATOM | 2770 | CG | GLN | A | 383 | 32.862 | 44.012 | 96.356 | 1.00 | 173.80 | A | C |
| ATOM | 2771 | CD | GLN | A | 383 | 33.886 | 43.062 | 96.950 | 1.00 | 174.39 | A | C |
| ATOM | 2772 | OE1 | GLN | A | 383 | 34.830 | 42.647 | 96.275 | 1.00 | 174.94 | A | O |
| ATOM | 2773 | NE2 | GLN | A | 383 | 33.697 | 42.703 | 98.215 | 1.00 | 174.37 | A | N |
| ATOM | 2774 | C | GLN | A | 383 | 30.644 | 41.563 | 94.283 | 1.00 | 170.46 | A | C |
| ATOM | 2775 | O | GLN | A | 383 | 29.592 | 41.635 | 94.918 | 1.00 | 170.54 | A | O |
| ATOM | 2776 | N | THR | A | 384 | 30.725 | 41.022 | 93.070 | 1.00 | 168.52 | A | N |
| ATOM | 2777 | CA | THR | A | 384 | 29.558 | 40.469 | 92.387 | 1.00 | 166.49 | A | C |
| ATOM | 2778 | CB | THR | A | 384 | 29.896 | 40.084 | 90.924 | 1.00 | 165.89 | A | C |
| ATOM | 2779 | OG1 | THR | A | 384 | 30.419 | 41.226 | 90.232 | 1.00 | 164.93 | A | O |
| ATOM | 2780 | CG2 | THR | A | 384 | 28.651 | 39.597 | 90.201 | 1.00 | 165.23 | A | C |
| ATOM | 2781 | C | THR | A | 384 | 29.033 | 39.232 | 93.118 | 1.00 | 165.71 | A | C |
| ATOM | 2782 | O | THR | A | 384 | 29.652 | 38.169 | 93.086 | 1.00 | 165.04 | A | O |
| ATOM | 2783 | N | ILE | A | 385 | 27.887 | 39.385 | 93.776 | 1.00 | 165.19 | A | N |
| ATOM | 2784 | CA | ILE | A | 385 | 27.260 | 38.301 | 94.529 | 1.00 | 164.81 | A | C |
| ATOM | 2785 | CB | ILE | A | 385 | 26.922 | 38.757 | 95.970 | 1.00 | 163.43 | A | C |
| ATOM | 2786 | CG2 | ILE | A | 385 | 26.567 | 37.552 | 96.830 | 1.00 | 162.84 | A | C |
| ATOM | 2787 | CG1 | ILE | A | 385 | 28.120 | 39.488 | 96.581 | 1.00 | 161.93 | A | C |
| ATOM | 2788 | CD1 | ILE | A | 385 | 27.829 | 40.149 | 97.915 | 1.00 | 160.16 | A | C |
| ATOM | 2789 | C | ILE | A | 385 | 25.965 | 37.876 | 93.833 | 1.00 | 165.79 | A | C |
| ATOM | 2790 | O | ILE | A | 385 | 24.972 | 37.551 | 94.485 | 1.00 | 165.50 | A | O |
| ATOM | 2791 | N | ALA | A | 386 | 25.987 | 37.878 | 92.504 | 1.00 | 167.16 | A | N |
| ATOM | 2792 | CA | ALA | A | 386 | 24.819 | 37.515 | 91.707 | 1.00 | 168.95 | A | C |
| ATOM | 2793 | CB | ALA | A | 386 | 25.176 | 37.538 | 90.224 | 1.00 | 168.52 | A | C |
| ATOM | 2794 | C | ALA | A | 386 | 24.201 | 36.165 | 92.072 | 1.00 | 170.48 | A | C |
| ATOM | 2795 | O | ALA | A | 386 | 24.902 | 35.184 | 92.315 | 1.00 | 170.51 | A | O |
| ATOM | 2796 | N | ALA | A | 387 | 22.872 | 36.144 | 92.097 | 1.00 | 172.47 | A | N |
| ATOM | 2797 | CA | ALA | A | 387 | 22.076 | 34.960 | 92.413 | 1.00 | 174.53 | A | C |
| ATOM | 2798 | CB | ALA | A | 387 | 22.341 | 34.505 | 93.845 | 1.00 | 174.07 | A | C |
| ATOM | 2799 | C | ALA | A | 387 | 20.621 | 35.392 | 92.253 | 1.00 | 176.18 | A | C |
| ATOM | 2800 | O | ALA | A | 387 | 20.049 | 36.000 | 93.158 | 1.00 | 176.41 | A | O |
| ATOM | 2801 | N | ALA | A | 388 | 20.040 | 35.080 | 91.095 | 1.00 | 178.05 | A | N |
| ATOM | 2802 | CA | ALA | A | 388 | 18.664 | 35.454 | 90.761 | 1.00 | 179.71 | A | C |
| ATOM | 2803 | CB | ALA | A | 388 | 17.738 | 35.240 | 91.957 | 1.00 | 179.67 | A | C |
| ATOM | 2804 | C | ALA | A | 388 | 18.680 | 36.926 | 90.352 | 1.00 | 180.89 | A | C |
| ATOM | 2805 | O | ALA | A | 388 | 18.809 | 37.813 | 91.197 | 1.00 | 181.28 | A | O |
| ATOM | 2806 | N | GLY | A | 389 | 18.552 | 37.174 | 89.051 | 1.00 | 181.96 | A | N |
| ATOM | 2807 | CA | GLY | A | 389 | 18.586 | 38.535 | 88.540 | 1.00 | 183.22 | A | C |
| ATOM | 2808 | C | GLY | A | 389 | 17.322 | 39.369 | 88.643 | 1.00 | 183.89 | A | C |
| ATOM | 2809 | O | GLY | A | 389 | 16.368 | 39.007 | 89.332 | 1.00 | 184.35 | A | O |
| ATOM | 2810 | N | ASP | A | 390 | 17.329 | 40.502 | 87.945 | 1.00 | 184.10 | A | N |
| ATOM | 2811 | CA | ASP | A | 390 | 16.204 | 41.431 | 87.928 | 1.00 | 183.78 | A | C |
| ATOM | 2812 | CB | ASP | A | 390 | 16.713 | 42.877 | 87.826 | 1.00 | 184.91 | A | C |
| ATOM | 2813 | CG | ASP | A | 390 | 18.028 | 42.990 | 87.065 | 1.00 | 185.73 | A | C |
| ATOM | 2814 | OD1 | ASP | A | 390 | 18.394 | 42.041 | 86.343 | 1.00 | 186.33 | A | O |
| ATOM | 2815 | OD2 | ASP | A | 390 | 18.696 | 44.039 | 87.182 | 1.00 | 186.08 | A | O |
| ATOM | 2816 | C | ASP | A | 390 | 15.199 | 41.160 | 86.805 | 1.00 | 182.86 | A | C |
| ATOM | 2817 | O | ASP | A | 390 | 14.153 | 41.805 | 86.738 | 1.00 | 182.68 | A | O |
| ATOM | 2818 | N | TRP | A | 391 | 15.518 | 40.214 | 85.925 | 1.00 | 181.67 | A | N |
| ATOM | 2819 | CA | TRP | A | 391 | 14.625 | 39.865 | 84.821 | 1.00 | 180.34 | A | C |
| ATOM | 2820 | CB | TRP | A | 391 | 15.310 | 40.111 | 83.467 | 1.00 | 179.46 | A | C |
| ATOM | 2821 | CG | TRP | A | 391 | 15.954 | 41.468 | 83.331 | 1.00 | 178.64 | A | C |
| ATOM | 2822 | CD2 | TRP | A | 391 | 15.455 | 42.589 | 82.587 | 1.00 | 178.13 | A | C |
| ATOM | 2823 | CE2 | TRP | A | 391 | 16.376 | 43.648 | 82.758 | 1.00 | 177.93 | A | C |
| ATOM | 2824 | CE3 | TRP | A | 391 | 14.321 | 42.802 | 81.793 | 1.00 | 177.71 | A | C |
| ATOM | 2825 | CD1 | TRP | A | 391 | 17.121 | 41.882 | 83.903 | 1.00 | 178.54 | A | C |
| ATOM | 2826 | NE1 | TRP | A | 391 | 17.382 | 43.189 | 83.565 | 1.00 | 178.17 | A | N |
| ATOM | 2827 | CZ2 | TRP | A | 391 | 16.197 | 44.904 | 82.164 | 1.00 | 177.28 | A | C |
| ATOM | 2828 | CZ3 | TRP | A | 391 | 14.143 | 44.051 | 81.202 | 1.00 | 177.26 | A | C |
| ATOM | 2829 | CH2 | TRP | A | 391 | 15.078 | 45.085 | 81.392 | 1.00 | 176.94 | A | C |
| ATOM | 2830 | C | TRP | A | 391 | 14.210 | 38.395 | 84.925 | 1.00 | 179.75 | A | C |
| ATOM | 2831 | O | TRP | A | 391 | 14.954 | 37.567 | 85.453 | 1.00 | 179.66 | A | O |
| ATOM | 2832 | N | CYS | A | 392 | 13.017 | 38.078 | 84.427 | 1.00 | 178.93 | A | N |
| ATOM | 2833 | CA | CYS | A | 392 | 12.508 | 36.710 | 84.460 | 1.00 | 177.89 | A | C |
| ATOM | 2834 | CB | CYS | A | 392 | 11.262 | 36.614 | 85.345 | 1.00 | 177.18 | A | C |
| ATOM | 2835 | SG | CYS | A | 392 | 9.761 | 37.286 | 84.599 | 1.00 | 176.10 | A | S |
| ATOM | 2836 | C | CYS | A | 392 | 12.161 | 36.266 | 83.045 | 1.00 | 177.63 | A | C |
| ATOM | 2837 | O | CYS | A | 392 | 12.539 | 36.991 | 82.101 | 1.00 | 177.28 | A | O |
| ATOM | 2838 | OXT | CYS | A | 392 | 11.528 | 35.199 | 82.900 | 1.00 | 177.56 | A | O |
| TER | 2839 | | CYS | A | 392 | | | | | | A | |
| ATOM | 2840 | O | HOH | W | 1 | 32.527 | 56.384 | 63.076 | 1.00 | 29.80 | W | O |
| ATOM | 2841 | O | HOH | W | 2 | 54.385 | 43.175 | 79.708 | 1.00 | 43.04 | W | O |
| ATOM | 2842 | O | HOH | W | 3 | 30.356 | 55.888 | 64.782 | 1.00 | 28.98 | W | O |
| ATOM | 2843 | O | HOH | W | 4 | 36.118 | 39.675 | 60.617 | 1.00 | 29.03 | W | O |
| ATOM | 2844 | O | HOH | W | 5 | 28.844 | 53.763 | 65.773 | 1.00 | 27.39 | W | O |
| ATOM | 2845 | O | HOH | W | 6 | 39.842 | 62.345 | 67.003 | 1.00 | 45.21 | W | O |
| ATOM | 2846 | O | HOH | W | 7 | 28.130 | 51.576 | 67.528 | 1.00 | 27.21 | W | O |
| ATOM | 2847 | O | HOH | W | 8 | 46.024 | 50.828 | 70.082 | 1.00 | 28.91 | W | O |
| ATOM | 2848 | O | HOH | W | 9 | 30.386 | 44.067 | 69.560 | 1.00 | 41.95 | W | O |
| ATOM | 2849 | O | HOH | W | 10 | 34.558 | 40.720 | 58.728 | 1.00 | 28.16 | W | O |
| ATOM | 2850 | O | HOH | W | 11 | 43.185 | 53.011 | 69.412 | 1.00 | 36.40 | W | O |
| ATOM | 2851 | O | HOH | W | 12 | 32.925 | 35.570 | 63.043 | 1.00 | 52.40 | W | O |
| ATOM | 2852 | O | HOH | W | 13 | 34.832 | 22.872 | 92.064 | 1.00 | 52.52 | W | O |
| ATOM | 2853 | O | HOH | W | 14 | 30.465 | 35.730 | 58.732 | 1.00 | 33.00 | W | O |
| ATOM | 2854 | O | HOH | W | 15 | 34.927 | 57.662 | 60.348 | 1.00 | 42.76 | W | O |
| ATOM | 2855 | O | HOH | W | 16 | 32.641 | 45.300 | 70.858 | 1.00 | 39.61 | W | O |
| ATOM | 2856 | O | HOH | W | 17 | 33.519 | 37.225 | 52.530 | 1.00 | 36.35 | W | O |
| ATOM | 2857 | O | HOH | W | 18 | 43.815 | 57.175 | 67.352 | 1.00 | 32.64 | W | O |
| ATOM | 2858 | O | HOH | W | 19 | 33.696 | 59.371 | 68.412 | 1.00 | 45.20 | W | O |
| ATOM | 2859 | O | HOH | W | 20 | 37.134 | 45.456 | 76.200 | 1.00 | 44.76 | W | O |
| ATOM | 2860 | O | HOH | W | 21 | 37.045 | 36.384 | 46.615 | 1.00 | 45.37 | W | O |
| ATOM | 2861 | O | HOH | W | 22 | 43.260 | 57.430 | 80.323 | 1.00 | 54.20 | W | O |
| ATOM | 2862 | O | HOH | W | 23 | 43.181 | 52.079 | 48.646 | 1.00 | 54.70 | W | O |
| ATOM | 2863 | O | HOH | W | 24 | 40.396 | 65.037 | 55.538 | 1.00 | 45.36 | W | O |
| ATOM | 2864 | O | HOH | W | 25 | 48.431 | 41.972 | 74.461 | 1.00 | 40.54 | W | O |
| ATOM | 2865 | O | HOH | W | 26 | 68.291 | 55.086 | 78.180 | 1.00 | 50.70 | W | O |
| ATOM | 2866 | O | HOH | W | 27 | 57.490 | 58.746 | 72.332 | 1.00 | 45.82 | W | O |
| ATOM | 2867 | O | HOH | W | 28 | 51.755 | 37.566 | 76.259 | 1.00 | 44.25 | W | O |
| ATOM | 2868 | O | HOH | W | 29 | 38.847 | 29.660 | 66.206 | 1.00 | 54.57 | W | O |
| ATOM | 2869 | O | HOH | W | 30 | 26.456 | 48.412 | 54.771 | 1.00 | 46.46 | W | O |
| ATOM | 2870 | O | HOH | W | 31 | 42.122 | 55.811 | 68.616 | 1.00 | 34.55 | W | O |
| ATOM | 2871 | O | HOH | W | 32 | 46.080 | 60.934 | 63.980 | 1.00 | 41.78 | W | O |
| ATOM | 2872 | O | HOH | W | 33 | 38.200 | 43.202 | 75.237 | 1.00 | 48.03 | W | O |
| ATOM | 2873 | O | HOH | W | 34 | 49.735 | 60.540 | 90.616 | 1.00 | 50.10 | W | O |
| ATOM | 2874 | O | HOH | W | 35 | 34.741 | 55.058 | 73.510 | 1.00 | 55.87 | W | O |
| ATOM | 2875 | O | HOH | W | 36 | 60.850 | 53.519 | 97.859 | 1.00 | 52.99 | W | O |
| ATOM | 2876 | O | HOH | W | 37 | 46.520 | 64.255 | 63.204 | 1.00 | 52.70 | W | O |
| ATOM | 2877 | O | HOH | W | 38 | 39.712 | 64.211 | 60.481 | 1.00 | 40.87 | W | O |
| ATOM | 2878 | O | HOH | W | 39 | 28.196 | 50.781 | 59.499 | 1.00 | 38.84 | W | O |
| ATOM | 2879 | O | HOH | W | 40 | 31.393 | 34.687 | 60.899 | 1.00 | 46.03 | W | O |
| ATOM | 2880 | O | HOH | W | 41 | 26.295 | 34.319 | 52.205 | 1.00 | 45.35 | W | O |
| ATOM | 2881 | O | HOH | W | 42 | 43.701 | 31.476 | 58.694 | 1.00 | 54.35 | W | O |
| ATOM | 2882 | O | HOH | W | 43 | 35.853 | 38.654 | 63.349 | 1.00 | 34.34 | W | O |
| ATOM | 2883 | O | HOH | W | 44 | 50.896 | 42.980 | 66.409 | 1.00 | 54.96 | W | O |
| ATOM | 2884 | O | HOH | W | 45 | 52.746 | 45.269 | 69.571 | 1.00 | 55.70 | W | O |
| ATOM | 2885 | O | HOH | W | 46 | 26.745 | 48.298 | 70.211 | 1.00 | 51.98 | W | O |
| ATOM | 2886 | O | HOH | W | 47 | 41.460 | 30.765 | 62.913 | 1.00 | 58.16 | W | O |
| ATOM | 2887 | O | HOH | W | 48 | 44.412 | 62.359 | 55.974 | 1.00 | 41.00 | W | O |
| ATOM | 2888 | O | HOH | W | 49 | 33.190 | 28.957 | 59.989 | 1.00 | 58.08 | W | O |
| ATOM | 2889 | O | HOH | W | 50 | 43.159 | 57.397 | 47.972 | 1.00 | 53.53 | W | O |
| ATOM | 2890 | O | HOH | W | 51 | 51.514 | 58.268 | 87.058 | 1.00 | 48.66 | W | O |
| ATOM | 2891 | O | HOH | W | 52 | 43.656 | 30.282 | 61.039 | 1.00 | 52.91 | W | O |
| ATOM | 2892 | O | HOH | W | 53 | 35.942 | 35.875 | 80.355 | 1.00 | 43.29 | W | O |
| ATOM | 2893 | O | HOH | W | 54 | 28.030 | 52.842 | 57.151 | 1.00 | 40.34 | W | O |
| ATOM | 2894 | O | HOH | W | 55 | 32.955 | 64.144 | 59.137 | 1.00 | 48.09 | W | O |
| ATOM | 2895 | O | HOH | W | 56 | 51.373 | 39.763 | 73.966 | 1.00 | 48.44 | W | O |
| ATOM | 2896 | O | HOH | W | 57 | 46.415 | 49.672 | 87.118 | 1.00 | 47.03 | W | O |
| ATOM | 2897 | O | HOH | W | 58 | 28.785 | 41.625 | 56.265 | 1.00 | 41.15 | W | O |
| ATOM | 2898 | O | HOH | W | 59 | 65.675 | 42.269 | 98.357 | 1.00 | 51.77 | W | O |
| ATOM | 2899 | O | HOH | W | 60 | 40.010 | 61.425 | 73.610 | 1.00 | 48.56 | W | O |
| TER | 2900 | | HOH | W | 60 | | | | | | W | |
| ATOM | 2901 | C1 | HET | B | 1 | 56.265 | 63.832 | 78.005 | 1.00 | 86.83 | B | C |
| ATOM | 2902 | S1 | HET | B | 1 | 55.495 | 65.396 | 78.330 | 1.00 | 90.47 | B | S |
| ATOM | 2903 | C2 | HET | B | 1 | 57.282 | 63.337 | 78.864 | 1.00 | 83.51 | B | C |
| ATOM | 2904 | C3 | HET | B | 1 | 55.843 | 63.103 | 76.881 | 1.00 | 84.30 | B | C |
| ATOM | 2905 | N1 | HET | B | 1 | 54.438 | 65.250 | 79.755 | 1.00 | 95.09 | B | N |
| ATOM | 2906 | O1 | HET | B | 1 | 56.497 | 66.369 | 78.689 | 1.00 | 90.98 | B | O |
| ATOM | 2907 | O2 | HET | B | 1 | 54.613 | 65.713 | 77.210 | 1.00 | 88.82 | B | O |
| ATOM | 2908 | C4 | HET | B | 1 | 57.899 | 62.071 | 78.593 | 1.00 | 81.65 | B | C |
| ATOM | 2909 | C5 | HET | B | 1 | 56.425 | 61.863 | 76.598 | 1.00 | 81.24 | B | C |
| ATOM | 2910 | C6 | HET | B | 1 | 53.490 | 66.303 | 80.043 | 1.00 | 99.89 | B | C |
| ATOM | 2911 | C7 | HET | B | 1 | 54.722 | 64.324 | 80.908 | 1.00 | 94.76 | B | C |
| ATOM | 2912 | C8 | HET | B | 1 | 57.456 | 61.321 | 77.438 | 1.00 | 79.44 | B | C |
| ATOM | 2913 | C9 | HET | B | 1 | 59.010 | 61.587 | 79.503 | 1.00 | 81.62 | B | C |
| ATOM | 2914 | C10 | HET | B | 1 | 53.957 | 67.641 | 80.186 | 1.00 | 101.63 | B | C |
| ATOM | 2915 | C11 | HET | B | 1 | 52.088 | 66.040 | 80.128 | 1.00 | 102.05 | B | C |
| ATOM | 2916 | C12 | HET | B | 1 | 53.693 | 63.159 | 81.087 | 1.00 | 93.15 | B | C |
| ATOM | 2917 | N2 | HET | B | 1 | 58.048 | 60.113 | 77.181 | 1.00 | 74.52 | B | N |
| ATOM | 2918 | C13 | HET | B | 1 | 53.050 | 68.704 | 80.408 | 1.00 | 102.96 | B | C |
| ATOM | 2919 | C14 | HET | B | 1 | 51.183 | 67.108 | 80.350 | 1.00 | 103.98 | B | C |
| ATOM | 2920 | C15 | HET | B | 1 | 58.046 | 59.394 | 76.028 | 1.00 | 72.20 | B | C |
| ATOM | 2921 | C16 | HET | B | 1 | 51.667 | 68.440 | 80.491 | 1.00 | 103.61 | B | C |
| ATOM | 2922 | C17 | HET | B | 1 | 58.573 | 57.948 | 76.253 | 1.00 | 70.10 | B | C |
| ATOM | 2923 | O3 | HET | B | 1 | 57.669 | 59.783 | 74.908 | 1.00 | 73.02 | B | O |
| ATOM | 2924 | C18 | HET | B | 1 | 57.563 | 57.070 | 77.039 | 1.00 | 68.00 | B | C |
| ATOM | 2925 | C19 | HET | B | 1 | 58.915 | 57.376 | 74.863 | 1.00 | 70.54 | B | C |
| ATOM | 2926 | O4 | HET | B | 1 | 59.740 | 57.968 | 77.072 | 1.00 | 67.93 | B | O |
| ATOM | 2927 | F1 | HET | B | 1 | 58.084 | 55.843 | 77.204 | 1.00 | 66.07 | B | F |
| ATOM | 2928 | F2 | HET | B | 1 | 57.298 | 57.618 | 78.214 | 1.00 | 67.27 | B | F |
| ATOM | 2929 | F3 | HET | B | 1 | 56.447 | 56.974 | 76.359 | 1.00 | 69.96 | B | F |
| TER | 2930 | | HET | B | 1 | | | | | | B | |
| END | | | | | | | | | | | | |

TABLE 7
Atomic coordinates for PDHK-2* with Compound 3

REMARK coordinates from restrained individual B-factor refinement
REMARK refinement resolution: 30 − 2.4 A

| | | |
|---|---|---|
| REMARK starting | r = 0.2144 | free_r = 0.2456 |
| REMARK final | r = 0.2111 | free_r = 0.2412 |

| | | |
|---|---|---|
| REMARK B rmsd for bonded mainchain atoms = | 1.652 | target = 1.5 |
| REMARK B rmsd for bonded sidechain atoms = | 2.441 | target = 2.0 |
| REMARK B rmsd for angle mainchain atoms = | 2.793 | target = 2.0 |
| REMARK B rmsd for angle sidechain atoms = | 3.648 | target = 2.5 |

REMARK wa = 2.24074
REMARK rweight = 8.765414E−02
REMARK target = mlf steps = 20
REMARK sg = P6(4) a = 108.876 b = 108.876 c = 84.025 alpha = 90 beta = 90 gamma = 120
REMARK parameter file 1: MSI_CNX_TOPPAR: protein_rep.param
REMARK parameter file 2: ../toppar/het.par
REMARK parameter file 3: MSI_CNX_TOPPAR: water_rep.param
REMARK molecular structure file: ../generate/generate.psf
REMARK input coordinates: minimize. pdb
REMARK reflection file = ../pdhk95.cv
REMARK ncs = none
REMARK B-correction resolution: 6.0 − 2.4
REMARK initial B-factor correction applied to fobs:

| | | | | | | |
|---|---|---|---|---|---|---|
| REMARK | B11 = | −2.607 | B22 = | −2.607 | B33 = | 5.214 |
| REMARK | B12 = | −3.645 | B13 = | 0.000 | B23 = | 0.000 |

REMARK B-factor correction applied to coordinate array B: −0.056
REMARK bulk solvent: (Mask) density level = 0.340465 e/A\hat{ }3, B-factor = 41.1384 A\hat{ }2
REMARK reflections with |Fobs|/sigma F < 0.0 rejected
REMARK reflections with |Fobs| > 10000 * rms(Fobs) rejected

| | | |
|---|---|---|
| REMARK theoretical total number of refl. in resol. range: | 22237 | (100.0%) |
| REMARK number of unobserved reflections (no entry or \|F\| = 0): | 1210 | (5.4%) |
| REMARK number of reflections rejected: | 0 | (0.0%) |
| REMARK total number of reflections used: | 21027 | (94.6%) |
| REMARK number of reflections in working set: | 20010 | (90.0%) |
| REMARK number of reflections in test set: | 1017 | (4.6%) |

REMARK FILENAME = “bindividual.pdb”
REMARK DATE: Apr. 03, 2001 18:40:35 created by user: knoechel
REMARK Written by CNX VERSION: 2000

| | | | | | | | | | | | | |
|---|---|---|---|---|---|---|---|---|---|---|---|---|
| ATOM | 1 | C | GLY | A | 14 | 61.366 | 42.481 | 101.253 | 1.00 | 44.49 | A | C |
| ATOM | 2 | O | GLY | A | 14 | 60.691 | 42.638 | 102.266 | 1.00 | 45.03 | A | O |
| ATOM | 3 | N | GLY | A | 14 | 63.521 | 41.536 | 100.474 | 1.00 | 45.59 | A | N |
| ATOM | 4 | CA | GLY | A | 14 | 62.203 | 41.231 | 101.076 | 1.00 | 43.39 | A | C |
| ATOM | 5 | N | SER | A | 15 | 61.399 | 43.382 | 100.276 | 1.00 | 45.96 | A | N |
| ATOM | 6 | CA | SER | A | 15 | 60.610 | 44.609 | 100.375 | 1.00 | 48.00 | A | C |
| ATOM | 7 | CB | SER | A | 15 | 61.530 | 45.812 | 100.663 | 1.00 | 48.81 | A | C |
| ATOM | 8 | OG | SER | A | 15 | 62.629 | 45.867 | 99.771 | 1.00 | 53.95 | A | O |
| ATOM | 9 | C | SER | A | 15 | 59.721 | 44.902 | 99.159 | 1.00 | 47.39 | A | C |
| ATOM | 10 | O | SER | A | 15 | 59.377 | 46.053 | 98.904 | 1.00 | 47.88 | A | O |
| ATOM | 11 | N | ALA | A | 16 | 59.339 | 43.868 | 98.414 | 1.00 | 47.50 | A | N |
| ATOM | 12 | CA | ALA | A | 16 | 58.475 | 44.073 | 97.249 | 1.00 | 47.40 | A | C |
| ATOM | 13 | CB | ALA | A | 16 | 58.333 | 42.780 | 96.447 | 1.00 | 47.36 | A | C |
| ATOM | 14 | C | ALA | A | 16 | 57.091 | 44.588 | 97.651 | 1.00 | 47.62 | A | C |
| ATOM | 15 | O | ALA | A | 16 | 56.561 | 45.508 | 97.020 | 1.00 | 49.07 | A | O |
| ATOM | 16 | N | PRO | A | 17 | 56.480 | 43.999 | 98.697 | 1.00 | 46.45 | A | N |
| ATOM | 17 | CD | PRO | A | 17 | 56.882 | 42.767 | 99.401 | 1.00 | 45.61 | A | C |
| ATOM | 18 | CA | PRO | A | 17 | 55.150 | 44.443 | 99.141 | 1.00 | 46.05 | A | C |
| ATOM | 19 | CB | PRO | A | 17 | 54.859 | 43.534 | 100.336 | 1.00 | 45.48 | A | C |
| ATOM | 20 | CG | PRO | A | 17 | 55.565 | 42.264 | 99.964 | 1.00 | 44.59 | A | C |
| ATOM | 21 | C | PRO | A | 17 | 55.111 | 45.918 | 99.521 | 1.00 | 46.28 | A | C |
| ATOM | 22 | O | PRO | A | 17 | 54.108 | 46.597 | 99.300 | 1.00 | 46.35 | A | O |
| ATOM | 23 | N | LYS | A | 18 | 56.208 | 46.404 | 100.094 | 1.00 | 47.28 | A | N |
| ATOM | 24 | CA | LYS | A | 18 | 56.315 | 47.801 | 100.506 | 1.00 | 48.48 | A | C |
| ATOM | 25 | CB | LYS | A | 18 | 57.635 | 48.033 | 101.253 | 1.00 | 48.36 | A | C |
| ATOM | 26 | CG | LYS | A | 18 | 57.891 | 49.472 | 101.692 | 1.00 | 49.37 | A | C |
| ATOM | 27 | CD | LYS | A | 18 | 59.267 | 49.602 | 102.327 | 1.00 | 50.39 | A | C |
| ATOM | 28 | CE | LYS | A | 18 | 59.580 | 51.037 | 102.698 | 1.00 | 51.42 | A | C |
| ATOM | 29 | NZ | LYS | A | 18 | 61.005 | 51.173 | 103.091 | 1.00 | 52.39 | A | N |
| ATOM | 30 | C | LYS | A | 18 | 56.265 | 48.686 | 99.268 | 1.00 | 48.64 | A | C |
| ATOM | 31 | O | LYS | A | 18 | 55.469 | 49.624 | 99.188 | 1.00 | 47.99 | A | O |
| ATOM | 32 | N | TYR | A | 19 | 57.127 | 48.376 | 98.306 | 1.00 | 48.44 | A | N |
| ATOM | 33 | CA | TYR | A | 19 | 57.192 | 49.127 | 97.062 | 1.00 | 49.99 | A | C |
| ATOM | 34 | CB | TYR | A | 19 | 58.205 | 48.503 | 96.117 | 1.00 | 52.71 | A | C |
| ATOM | 35 | CG | TYR | A | 19 | 59.606 | 48.434 | 96.654 | 1.00 | 55.83 | A | C |
| ATOM | 36 | CD1 | TYR | A | 19 | 60.126 | 49.464 | 97.435 | 1.00 | 57.08 | A | C |
| ATOM | 37 | CE1 | TYR | A | 19 | 61.457 | 49.456 | 97.835 | 1.00 | 59.63 | A | C |
| ATOM | 38 | CD2 | TYR | A | 19 | 60.449 | 47.385 | 96.297 | 1.00 | 57.60 | A | C |
| ATOM | 39 | CE2 | TYR | A | 19 | 61.780 | 47.364 | 96.691 | 1.00 | 59.89 | A | C |
| ATOM | 40 | CZ | TYR | A | 19 | 62.279 | 48.402 | 97.456 | 1.00 | 60.28 | A | C |
| ATOM | 41 | OH | TYR | A | 19 | 63.605 | 48.392 | 97.822 | 1.00 | 62.84 | A | O |
| ATOM | 42 | C | TYR | A | 19 | 55.845 | 49.147 | 96.368 | 1.00 | 49.21 | A | C |
| ATOM | 43 | O | TYR | A | 19 | 55.365 | 50.198 | 95.950 | 1.00 | 49.53 | A | O |
| ATOM | 44 | N | ILE | A | 20 | 55.247 | 47.972 | 96.225 | 1.00 | 47.86 | A | N |
| ATOM | 45 | CA | ILE | A | 20 | 53.945 | 47.867 | 95.590 | 1.00 | 47.25 | A | C |
| ATOM | 46 | CB | ILE | A | 20 | 53.407 | 46.425 | 95.656 | 1.00 | 44.48 | A | C |
| ATOM | 47 | CG2 | ILE | A | 20 | 51.990 | 46.368 | 95.114 | 1.00 | 41.84 | A | C |
| ATOM | 48 | CG1 | ILE | A | 20 | 54.323 | 45.501 | 94.844 | 1.00 | 43.99 | A | C |
| ATOM | 49 | CD1 | ILE | A | 20 | 53.996 | 44.016 | 94.984 | 1.00 | 39.59 | A | C |
| ATOM | 50 | C | ILE | A | 20 | 52.966 | 48.794 | 96.288 | 1.00 | 48.14 | A | C |
| ATOM | 51 | O | ILE | A | 20 | 52.206 | 49.504 | 95.636 | 1.00 | 48.93 | A | O |
| ATOM | 52 | N | GLU | A | 21 | 52.996 | 48.789 | 97.617 | 1.00 | 47.97 | A | N |
| ATOM | 53 | CA | GLU | A | 21 | 52.105 | 49.632 | 98.398 | 1.00 | 48.47 | A | C |
| ATOM | 54 | CB | GLU | A | 21 | 52.252 | 49.313 | 99.884 | 1.00 | 50.57 | A | C |
| ATOM | 55 | CG | GLU | A | 21 | 51.110 | 49.832 | 100.745 | 1.00 | 54.60 | A | C |
| ATOM | 56 | CD | GLU | A | 21 | 49.752 | 49.330 | 100.271 | 1.00 | 57.06 | A | C |
| ATOM | 57 | OE1 | GLU | A | 21 | 49.619 | 48.113 | 99.996 | 1.00 | 58.08 | A | O |
| ATOM | 58 | OE2 | GLU | A | 21 | 48.817 | 50.156 | 100.180 | 1.00 | 58.61 | A | O |
| ATOM | 59 | C | GLU | A | 21 | 52.419 | 51.104 | 98.150 | 1.00 | 47.47 | A | C |
| ATOM | 60 | O | GLU | A | 21 | 51.518 | 51.935 | 98.029 | 1.00 | 48.57 | A | O |
| ATOM | 61 | N | HIS | A | 22 | 53.702 | 51.421 | 98.068 | 1.00 | 45.27 | A | N |
| ATOM | 62 | CA | HIS | A | 22 | 54.132 | 52.790 | 97.839 | 1.00 | 45.49 | A | C |
| ATOM | 63 | CB | HIS | A | 22 | 55.653 | 52.881 | 97.933 | 1.00 | 44.95 | A | C |
| ATOM | 64 | CG | HIS | A | 22 | 56.189 | 54.237 | 97.610 | 1.00 | 46.05 | A | C |
| ATOM | 65 | CD2 | HIS | A | 22 | 57.044 | 54.655 | 96.646 | 1.00 | 45.57 | A | C |
| ATOM | 66 | ND1 | HIS | A | 22 | 55.826 | 55.364 | 98.316 | 1.00 | 45.96 | A | N |
| ATOM | 67 | CE1 | HIS | A | 22 | 56.435 | 56.417 | 97.800 | 1.00 | 46.21 | A | C |
| ATOM | 68 | NE2 | HIS | A | 22 | 57.179 | 56.015 | 96.785 | 1.00 | 44.28 | A | N |
| ATOM | 69 | C | HIS | A | 22 | 53.684 | 53.332 | 96.487 | 1.00 | 45.03 | A | C |
| ATOM | 70 | O | HIS | A | 22 | 52.945 | 54.313 | 96.416 | 1.00 | 44.82 | A | O |
| ATOM | 71 | N | PHE | A | 23 | 54.145 | 52.694 | 95.415 | 1.00 | 45.48 | A | N |
| ATOM | 72 | CA | PHE | A | 23 | 53.809 | 53.131 | 94.068 | 1.00 | 44.43 | A | C |
| ATOM | 73 | CB | PHE | A | 23 | 54.724 | 52.451 | 93.051 | 1.00 | 41.34 | A | C |
| ATOM | 74 | CG | PHE | A | 23 | 56.157 | 52.885 | 93.153 | 1.00 | 40.22 | A | C |
| ATOM | 75 | CD1 | PHE | A | 23 | 57.109 | 52.057 | 93.742 | 1.00 | 41.23 | A | C |
| ATOM | 76 | CD2 | PHE | A | 23 | 56.558 | 54.135 | 92.678 | 1.00 | 39.61 | A | C |
| ATOM | 77 | CE1 | PHE | A | 23 | 58.442 | 52.464 | 93.863 | 1.00 | 39.22 | A | C |
| ATOM | 78 | CE2 | PHE | A | 23 | 57.889 | 54.559 | 92.792 | 1.00 | 36.85 | A | C |
| ATOM | 79 | CZ | PHE | A | 23 | 58.833 | 53.716 | 93.387 | 1.00 | 37.92 | A | C |
| ATOM | 80 | C | PHE | A | 23 | 52.347 | 52.952 | 93.674 | 1.00 | 44.87 | A | C |
| ATOM | 81 | O | PHE | A | 23 | 51.872 | 53.630 | 92.766 | 1.00 | 43.33 | A | O |
| ATOM | 82 | N | SER | A | 24 | 51.633 | 52.065 | 94.362 | 1.00 | 46.32 | A | N |
| ATOM | 83 | CA | SER | A | 24 | 50.224 | 51.852 | 94.063 | 1.00 | 47.81 | A | C |
| ATOM | 84 | CB | SER | A | 24 | 49.699 | 50.579 | 94.743 | 1.00 | 48.14 | A | C |
| ATOM | 85 | OG | SER | A | 24 | 49.591 | 50.733 | 96.148 | 1.00 | 49.25 | A | O |
| ATOM | 86 | C | SER | A | 24 | 49.420 | 53.060 | 94.538 | 1.00 | 49.30 | A | C |
| ATOM | 87 | O | SER | A | 24 | 48.254 | 53.215 | 94.184 | 1.00 | 50.23 | A | O |
| ATOM | 88 | N | LYS | A | 25 | 50.047 | 53.910 | 95.350 | 1.00 | 50.75 | A | N |
| ATOM | 89 | CA | LYS | A | 25 | 49.386 | 55.106 | 95.865 | 1.00 | 52.50 | A | C |
| ATOM | 90 | CB | LYS | A | 25 | 50.114 | 55.629 | 97.112 | 1.00 | 55.41 | A | C |
| ATOM | 91 | CG | LYS | A | 25 | 49.971 | 54.737 | 98.342 | 1.00 | 60.00 | A | C |
| ATOM | 92 | CD | LYS | A | 25 | 50.873 | 55.200 | 99.479 | 1.00 | 64.51 | A | C |
| ATOM | 93 | CE | LYS | A | 25 | 50.771 | 54.265 | 100.684 | 1.00 | 67.31 | A | C |
| ATOM | 94 | NZ | LYS | A | 25 | 51.790 | 54.584 | 101.730 | 1.00 | 69.16 | A | N |
| ATOM | 95 | C | LYS | A | 25 | 49.363 | 56.181 | 94.781 | 1.00 | 51.65 | A | C |
| ATOM | 96 | O | LYS | A | 25 | 48.655 | 57.177 | 94.894 | 1.00 | 50.90 | A | O |
| ATOM | 97 | N | PHE | A | 26 | 50.143 | 55.966 | 93.728 | 1.00 | 51.06 | A | N |
| ATOM | 98 | CA | PHE | A | 26 | 50.208 | 56.901 | 92.611 | 1.00 | 49.60 | A | C |
| ATOM | 99 | CB | PHE | A | 26 | 51.626 | 56.986 | 92.047 | 1.00 | 51.54 | A | C |
| ATOM | 100 | CG | PHE | A | 26 | 52.618 | 57.619 | 92.974 | 1.00 | 55.39 | A | C |
| ATOM | 101 | CD1 | PHE | A | 26 | 53.210 | 56.885 | 93.997 | 1.00 | 55.64 | A | C |
| ATOM | 102 | CD2 | PHE | A | 26 | 52.981 | 58.957 | 92.809 | 1.00 | 56.40 | A | C |
| ATOM | 103 | CE1 | PHE | A | 26 | 54.145 | 57.473 | 94.841 | 1.00 | 56.65 | A | C |
| ATOM | 104 | CE2 | PHE | A | 26 | 53.916 | 59.555 | 93.649 | 1.00 | 55.65 | A | C |
| ATOM | 105 | CZ | PHE | A | 26 | 54.500 | 58.812 | 94.665 | 1.00 | 56.60 | A | C |
| ATOM | 106 | C | PHE | A | 26 | 49.282 | 56.467 | 91.479 | 1.00 | 48.12 | A | C |
| ATOM | 107 | O | PHE | A | 26 | 49.013 | 55.277 | 91.298 | 1.00 | 47.06 | A | O |
| ATOM | 108 | N | SER | A | 27 | 48.810 | 57.443 | 90.714 | 1.00 | 45.31 | A | N |
| ATOM | 109 | CA | SER | A | 27 | 47.940 | 57.175 | 89.582 | 1.00 | 42.74 | A | C |
| ATOM | 110 | CB | SER | A | 27 | 46.839 | 58.232 | 89.491 | 1.00 | 42.33 | A | C |
| ATOM | 111 | OG | SER | A | 27 | 45.619 | 57.741 | 90.011 | 1.00 | 46.59 | A | O |
| ATOM | 112 | C | SER | A | 27 | 48.773 | 57.223 | 88.313 | 1.00 | 40.37 | A | C |
| ATOM | 113 | O | SER | A | 27 | 49.539 | 58.160 | 88.113 | 1.00 | 40.19 | A | O |
| ATOM | 114 | N | PRO | A | 28 | 48.662 | 56.199 | 87.452 | 1.00 | 38.55 | A | N |
| ATOM | 115 | CD | PRO | A | 28 | 47.911 | 54.937 | 87.564 | 1.00 | 37.29 | A | C |
| ATOM | 116 | CA | PRO | A | 28 | 49.453 | 56.240 | 86.216 | 1.00 | 36.57 | A | C |
| ATOM | 117 | CB | PRO | A | 28 | 49.099 | 54.914 | 85.523 | 1.00 | 37.58 | A | C |
| ATOM | 118 | CG | PRO | A | 28 | 47.798 | 54.518 | 86.120 | 1.00 | 38.89 | A | C |
| ATOM | 119 | C | PRO | A | 28 | 49.086 | 57.483 | 85.384 | 1.00 | 35.99 | A | C |
| ATOM | 120 | O | PRO | A | 28 | 47.934 | 57.914 | 85.377 | 1.00 | 34.65 | A | O |
| ATOM | 121 | N | SER | A | 29 | 50.070 | 58.055 | 84.693 | 1.00 | 35.56 | A | N |
| ATOM | 122 | CA | SER | A | 29 | 49.869 | 59.261 | 83.893 | 1.00 | 33.34 | A | C |
| ATOM | 123 | CB | SER | A | 29 | 51.046 | 60.213 | 84.098 | 1.00 | 32.80 | A | C |
| ATOM | 124 | OG | SER | A | 29 | 51.164 | 60.568 | 85.463 | 1.00 | 40.79 | A | O |
| ATOM | 125 | C | SER | A | 29 | 49.706 | 58.985 | 82.409 | 1.00 | 32.26 | A | C |
| ATOM | 126 | O | SER | A | 29 | 50.673 | 58.694 | 81.717 | 1.00 | 32.08 | A | O |
| ATOM | 127 | N | PRO | A | 30 | 48.480 | 59.103 | 81.892 | 1.00 | 31.54 | A | N |
| ATOM | 128 | CD | PRO | A | 30 | 47.217 | 59.412 | 82.585 | 1.00 | 31.43 | A | C |
| ATOM | 129 | CA | PRO | A | 30 | 48.255 | 58.847 | 80.470 | 1.00 | 32.23 | A | C |
| ATOM | 130 | CB | PRO | A | 30 | 46.731 | 58.805 | 80.367 | 1.00 | 32.36 | A | C |
| ATOM | 131 | CG | PRO | A | 30 | 46.293 | 59.752 | 81.435 | 1.00 | 32.31 | A | C |
| ATOM | 132 | C | PRO | A | 30 | 48.885 | 59.876 | 79.533 | 1.00 | 34.85 | A | C |
| ATOM | 133 | O | PRO | A | 30 | 48.734 | 61.086 | 79.719 | 1.00 | 36.33 | A | O |
| ATOM | 134 | N | LEU | A | 31 | 49.598 | 59.386 | 78.525 | 1.00 | 33.50 | A | N |
| ATOM | 135 | CA | LEU | A | 31 | 50.238 | 60.260 | 77.561 | 1.00 | 32.34 | A | C |
| ATOM | 136 | CB | LEU | A | 31 | 51.642 | 59.757 | 77.208 | 1.00 | 31.53 | A | C |
| ATOM | 137 | CG | LEU | A | 31 | 52.665 | 59.437 | 78.306 | 1.00 | 32.33 | A | C |
| ATOM | 138 | CD1 | LEU | A | 31 | 53.966 | 58.970 | 77.645 | 1.00 | 30.48 | A | C |
| ATOM | 139 | CD2 | LEU | A | 31 | 52.918 | 60.655 | 79.182 | 1.00 | 33.02 | A | C |
| ATOM | 140 | C | LEU | A | 31 | 49.402 | 60.318 | 76.293 | 1.00 | 35.09 | A | C |
| ATOM | 141 | O | LEU | A | 31 | 48.718 | 59.358 | 75.938 | 1.00 | 35.77 | A | O |
| ATOM | 142 | N | SER | A | 32 | 49.458 | 61.453 | 75.610 | 1.00 | 36.81 | A | N |
| ATOM | 143 | CA | SER | A | 32 | 48.713 | 61.627 | 74.373 | 1.00 | 38.16 | A | C |
| ATOM | 144 | CB | SER | A | 32 | 48.241 | 63.077 | 74.233 | 1.00 | 36.64 | A | C |
| ATOM | 145 | OG | SER | A | 32 | 49.347 | 63.933 | 74.020 | 1.00 | 35.95 | A | O |
| ATOM | 146 | C | SER | A | 32 | 49.642 | 61.281 | 73.225 | 1.00 | 39.39 | A | C |
| ATOM | 147 | O | SER | A | 32 | 50.862 | 61.229 | 73.385 | 1.00 | 38.91 | A | O |
| ATOM | 148 | N | MET | A | 33 | 49.068 | 61.039 | 72.061 | 1.00 | 42.36 | A | N |
| ATOM | 149 | CA | MET | A | 33 | 49.881 | 60.706 | 70.914 | 1.00 | 45.76 | A | C |
| ATOM | 150 | CB | MET | A | 33 | 49.005 | 60.434 | 69.706 | 1.00 | 47.28 | A | C |
| ATOM | 151 | CG | MET | A | 33 | 49.740 | 59.723 | 68.598 | 1.00 | 48.58 | A | C |
| ATOM | 152 | SD | MET | A | 33 | 49.562 | 57.949 | 68.731 | 1.00 | 48.72 | A | S |
| ATOM | 153 | CE | MET | A | 33 | 50.843 | 57.599 | 69.860 | 1.00 | 52.48 | A | C |
| ATOM | 154 | C | MET | A | 33 | 50.789 | 61.895 | 70.627 | 1.00 | 48.04 | A | C |
| ATOM | 155 | O | MET | A | 33 | 51.877 | 61.746 | 70.076 | 1.00 | 48.35 | A | O |
| ATOM | 156 | N | LYS | A | 34 | 50.340 | 63.081 | 71.014 | 1.00 | 49.77 | A | N |
| ATOM | 157 | CA | LYS | A | 34 | 51.120 | 64.288 | 70.797 | 1.00 | 51.40 | A | C |
| ATOM | 158 | CB | LYS | A | 34 | 50.247 | 65.509 | 71.086 | 1.00 | 55.68 | A | C |
| ATOM | 159 | CG | LYS | A | 34 | 50.211 | 66.530 | 69.952 | 1.00 | 59.38 | A | C |
| ATOM | 160 | CD | LYS | A | 34 | 51.520 | 67.296 | 69.875 | 1.00 | 62.52 | A | C |
| ATOM | 161 | CE | LYS | A | 34 | 51.860 | 67.907 | 71.229 | 1.00 | 61.77 | A | C |
| ATOM | 162 | NZ | LYS | A | 34 | 53.222 | 68.496 | 71.245 | 1.00 | 63.23 | A | N |
| ATOM | 163 | C | LYS | A | 34 | 52.355 | 64.270 | 71.695 | 1.00 | 50.51 | A | C |
| ATOM | 164 | O | LYS | A | 34 | 53.476 | 64.515 | 71.241 | 1.00 | 50.38 | A | O |
| ATOM | 165 | N | GLN | A | 35 | 52.141 | 63.973 | 72.973 | 1.00 | 48.80 | A | N |
| ATOM | 166 | CA | GLN | A | 35 | 53.230 | 63.895 | 73.934 | 1.00 | 46.72 | A | C |
| ATOM | 167 | CB | GLN | A | 35 | 52.687 | 63.570 | 75.322 | 1.00 | 47.12 | A | C |
| ATOM | 168 | CG | GLN | A | 35 | 51.875 | 64.697 | 75.926 | 1.00 | 47.83 | A | C |
| ATOM | 169 | CD | GLN | A | 35 | 51.217 | 64.299 | 77.223 | 1.00 | 45.73 | A | C |
| ATOM | 170 | OE1 | GLN | A | 35 | 50.368 | 63.409 | 77.253 | 1.00 | 46.60 | A | O |
| ATOM | 171 | NE2 | GLN | A | 35 | 51.606 | 64.956 | 78.306 | 1.00 | 45.56 | A | N |
| ATOM | 172 | C | GLN | A | 35 | 54.250 | 62.836 | 73.520 | 1.00 | 46.53 | A | C |
| ATOM | 173 | O | GLN | A | 35 | 55.455 | 63.046 | 73.678 | 1.00 | 46.77 | A | O |
| ATOM | 174 | N | PHE | A | 36 | 53.771 | 61.703 | 73.002 | 1.00 | 44.05 | A | N |
| ATOM | 175 | CA | PHE | A | 36 | 54.659 | 60.635 | 72.550 | 1.00 | 42.36 | A | C |
| ATOM | 176 | CB | PHE | A | 36 | 53.863 | 59.459 | 71.987 | 1.00 | 39.50 | A | C |
| ATOM | 177 | CG | PHE | A | 36 | 53.476 | 58.443 | 73.013 | 1.00 | 34.88 | A | C |
| ATOM | 178 | CD1 | PHE | A | 36 | 52.147 | 58.288 | 73.380 | 1.00 | 32.06 | A | C |
| ATOM | 179 | CD2 | PHE | A | 36 | 54.444 | 57.638 | 73.616 | 1.00 | 33.50 | A | C |
| ATOM | 180 | CE1 | PHE | A | 36 | 51.783 | 57.341 | 74.330 | 1.00 | 30.99 | A | C |
| ATOM | 181 | CE2 | PHE | A | 36 | 54.092 | 56.680 | 74.572 | 1.00 | 30.14 | A | C |
| ATOM | 182 | CZ | PHE | A | 36 | 52.762 | 56.534 | 74.928 | 1.00 | 30.10 | A | C |
| ATOM | 183 | C | PHE | A | 36 | 55.612 | 61.140 | 71.477 | 1.00 | 43.14 | A | C |
| ATOM | 184 | O | PHE | A | 36 | 56.792 | 60.794 | 71.476 | 1.00 | 39.82 | A | O |
| ATOM | 185 | N | LEU | A | 37 | 55.092 | 61.950 | 70.560 | 1.00 | 46.63 | A | N |
| ATOM | 186 | CA | LEU | A | 37 | 55.908 | 62.509 | 69.490 | 1.00 | 49.84 | A | C |
| ATOM | 187 | CB | LEU | A | 37 | 55.036 | 63.322 | 68.528 | 1.00 | 48.69 | A | C |
| ATOM | 188 | CG | LEU | A | 37 | 54.068 | 62.487 | 67.675 | 1.00 | 49.08 | A | C |
| ATOM | 189 | CD1 | LEU | A | 37 | 53.179 | 63.402 | 66.853 | 1.00 | 48.49 | A | C |
| ATOM | 190 | CD2 | LEU | A | 37 | 54.850 | 61.552 | 66.759 | 1.00 | 47.83 | A | C |
| ATOM | 191 | C | LEU | A | 37 | 57.003 | 63.383 | 70.094 | 1.00 | 52.93 | A | C |
| ATOM | 192 | O | LEU | A | 37 | 58.144 | 63.374 | 69.631 | 1.00 | 53.85 | A | O |
| ATOM | 193 | N | ASP | A | 38 | 56.652 | 64.125 | 71.142 | 1.00 | 54.87 | A | N |
| ATOM | 194 | CA | ASP | A | 38 | 57.610 | 64.985 | 71.823 | 1.00 | 56.61 | A | C |
| ATOM | 195 | CB | ASP | A | 38 | 56.893 | 65.842 | 72.864 | 1.00 | 57.62 | A | C |
| ATOM | 196 | CG | ASP | A | 38 | 56.274 | 67.096 | 72.261 | 1.00 | 59.03 | A | C |
| ATOM | 197 | OD1 | ASP | A | 38 | 55.693 | 67.024 | 71.151 | 1.00 | 57.76 | A | O |
| ATOM | 198 | OD2 | ASP | A | 38 | 56.368 | 68.156 | 72.913 | 1.00 | 60.76 | A | O |
| ATOM | 199 | C | ASP | A | 38 | 58.707 | 64.153 | 72.486 | 1.00 | 57.89 | A | C |
| ATOM | 200 | O | ASP | A | 38 | 59.881 | 64.297 | 72.150 | 1.00 | 58.25 | A | O |
| ATOM | 201 | N | PHE | A | 39 | 58.329 | 63.283 | 73.422 | 1.00 | 59.39 | A | N |
| ATOM | 202 | CA | PHE | A | 39 | 59.303 | 62.428 | 74.107 | 1.00 | 61.31 | A | C |
| ATOM | 203 | CB | PHE | A | 39 | 58.602 | 61.331 | 74.916 | 1.00 | 61.81 | A | C |
| ATOM | 204 | CG | PHE | A | 39 | 58.469 | 61.641 | 76.375 | 1.00 | 62.17 | A | C |
| ATOM | 205 | CD1 | PHE | A | 39 | 57.289 | 62.172 | 76.885 | 1.00 | 63.43 | A | C |
| ATOM | 206 | CD2 | PHE | A | 39 | 59.530 | 61.412 | 77.240 | 1.00 | 62.72 | A | C |
| ATOM | 207 | CE1 | PHE | A | 39 | 57.169 | 62.476 | 78.240 | 1.00 | 64.21 | A | C |
| ATOM | 208 | CE2 | PHE | A | 39 | 59.422 | 61.713 | 78.594 | 1.00 | 63.36 | A | C |
| ATOM | 209 | CZ | PHE | A | 39 | 58.238 | 62.245 | 79.097 | 1.00 | 64.26 | A | C |
| ATOM | 210 | C | PHE | A | 39 | 60.233 | 61.759 | 73.105 | 1.00 | 63.32 | A | C |
| ATOM | 211 | O | PHE | A | 39 | 61.393 | 61.482 | 73.404 | 1.00 | 62.04 | A | O |
| ATOM | 212 | N | GLY | A | 40 | 59.708 | 61.494 | 71.913 | 1.00 | 67.01 | A | N |
| ATOM | 213 | CA | GLY | A | 40 | 60.500 | 60.845 | 70.885 | 1.00 | 71.11 | A | C |
| ATOM | 214 | C | GLY | A | 40 | 61.136 | 61.809 | 69.904 | 1.00 | 74.50 | A | C |
| ATOM | 215 | O | GLY | A | 40 | 61.991 | 61.410 | 69.113 | 1.00 | 73.36 | A | O |
| ATOM | 216 | N | SER | A | 41 | 60.711 | 63.070 | 69.965 | 1.00 | 78.88 | A | N |
| ATOM | 217 | CA | SER | A | 41 | 61.210 | 64.136 | 69.097 | 1.00 | 83.89 | A | C |
| ATOM | 218 | CB | SER | A | 41 | 61.753 | 65.287 | 69.948 | 1.00 | 84.01 | A | C |
| ATOM | 219 | OG | SER | A | 41 | 62.382 | 66.266 | 69.137 | 1.00 | 85.03 | A | O |
| ATOM | 220 | C | SER | A | 41 | 62.293 | 63.657 | 68.142 | 1.00 | 87.02 | A | C |
| ATOM | 221 | O | SER | A | 41 | 62.010 | 63.011 | 67.129 | 1.00 | 88.90 | A | O |
| ATOM | 222 | N | SER | A | 42 | 63.535 | 63.980 | 68.477 | 1.00 | 89.38 | A | N |
| ATOM | 223 | CA | SER | A | 42 | 64.686 | 63.588 | 67.678 | 1.00 | 91.87 | A | C |
| ATOM | 224 | CB | SER | A | 42 | 64.798 | 64.478 | 66.430 | 1.00 | 92.05 | A | C |
| ATOM | 225 | OG | SER | A | 42 | 65.769 | 63.984 | 65.518 | 1.00 | 92.89 | A | O |
| ATOM | 226 | C | SER | A | 42 | 65.891 | 63.785 | 68.585 | 1.00 | 92.92 | A | C |
| ATOM | 227 | O | SER | A | 42 | 66.810 | 62.960 | 68.627 | 1.00 | 93.33 | A | O |
| ATOM | 228 | N | ASN | A | 43 | 65.858 | 64.884 | 69.331 | 1.00 | 93.53 | A | N |
| ATOM | 229 | CA | ASN | A | 43 | 66.933 | 65.221 | 70.248 | 1.00 | 93.40 | A | C |
| ATOM | 230 | CB | ASN | A | 43 | 67.572 | 66.551 | 69.845 | 1.00 | 95.79 | A | C |
| ATOM | 231 | CG | ASN | A | 43 | 68.377 | 66.445 | 68.557 | 1.00 | 97.68 | A | C |
| ATOM | 232 | OD1 | ASN | A | 43 | 68.919 | 67.436 | 68.071 | 1.00 | 98.23 | A | O |
| ATOM | 233 | ND2 | ASN | A | 43 | 68.462 | 65.236 | 68.004 | 1.00 | 98.31 | A | N |
| ATOM | 234 | C | ASN | A | 43 | 66.460 | 65.281 | 71.693 | 1.00 | 91.37 | A | C |
| ATOM | 235 | O | ASN | A | 43 | 67.274 | 65.464 | 72.600 | 1.00 | 91.23 | A | O |
| ATOM | 236 | N | ALA | A | 44 | 65.151 | 65.141 | 71.907 | 1.00 | 88.41 | A | N |
| ATOM | 237 | CA | ALA | A | 44 | 64.623 | 65.145 | 73.267 | 1.00 | 85.10 | A | C |
| ATOM | 238 | CB | ALA | A | 44 | 63.135 | 64.804 | 73.275 | 1.00 | 84.40 | A | C |
| ATOM | 239 | C | ALA | A | 44 | 65.420 | 64.043 | 73.945 | 1.00 | 82.80 | A | C |
| ATOM | 240 | O | ALA | A | 44 | 65.109 | 62.860 | 73.806 | 1.00 | 83.70 | A | O |
| ATOM | 241 | N | CYS | A | 45 | 66.471 | 64.440 | 74.651 | 1.00 | 79.03 | A | N |
| ATOM | 242 | CA | CYS | A | 45 | 67.339 | 63.492 | 75.330 | 1.00 | 74.33 | A | C |
| ATOM | 243 | CB | CYS | A | 45 | 68.185 | 64.220 | 76.373 | 1.00 | 74.91 | A | C |
| ATOM | 244 | SG | CYS | A | 45 | 67.226 | 65.347 | 77.385 | 1.00 | 76.70 | A | S |
| ATOM | 245 | C | CYS | A | 45 | 66.583 | 62.344 | 75.989 | 1.00 | 70.03 | A | C |
| ATOM | 246 | O | CYS | A | 45 | 65.393 | 62.443 | 76.299 | 1.00 | 68.12 | A | O |
| ATOM | 247 | N | GLU | A | 46 | 67.287 | 61.242 | 76.186 | 1.00 | 65.63 | A | N |
| ATOM | 248 | CA | GLU | A | 46 | 66.694 | 60.082 | 76.817 | 1.00 | 62.21 | A | C |
| ATOM | 249 | CB | GLU | A | 46 | 67.467 | 58.826 | 76.421 | 1.00 | 61.51 | A | C |
| ATOM | 250 | CG | GLU | A | 46 | 68.961 | 59.021 | 76.339 | 1.00 | 58.90 | A | C |
| ATOM | 251 | CD | GLU | A | 46 | 69.669 | 57.751 | 75.933 | 1.00 | 58.86 | A | C |
| ATOM | 252 | OE1 | GLU | A | 46 | 69.472 | 56.733 | 76.628 | 1.00 | 54.40 | A | O |
| ATOM | 253 | OE2 | GLU | A | 46 | 70.419 | 57.770 | 74.928 | 1.00 | 60.65 | A | O |
| ATOM | 254 | C | GLU | A | 46 | 66.716 | 60.271 | 78.319 | 1.00 | 59.40 | A | C |
| ATOM | 255 | O | GLU | A | 46 | 66.151 | 59.479 | 79.069 | 1.00 | 59.71 | A | O |
| ATOM | 256 | N | LYS | A | 47 | 67.380 | 61.335 | 78.749 | 1.00 | 56.83 | A | N |
| ATOM | 257 | CA | LYS | A | 47 | 67.474 | 61.648 | 80.164 | 1.00 | 54.64 | A | C |
| ATOM | 258 | CB | LYS | A | 47 | 68.353 | 62.880 | 80.384 | 1.00 | 56.58 | A | C |
| ATOM | 259 | CG | LYS | A | 47 | 69.714 | 62.802 | 79.728 | 1.00 | 60.71 | A | C |
| ATOM | 260 | CD | LYS | A | 47 | 70.512 | 64.080 | 79.966 | 1.00 | 65.52 | A | C |
| ATOM | 261 | CE | LYS | A | 47 | 69.839 | 65.298 | 79.342 | 1.00 | 66.95 | A | C |
| ATOM | 262 | NZ | LYS | A | 47 | 70.626 | 66.543 | 79.567 | 1.00 | 68.68 | A | N |
| ATOM | 263 | C | LYS | A | 47 | 66.065 | 61.945 | 80.644 | 1.00 | 51.95 | A | C |
| ATOM | 264 | O | LYS | A | 47 | 65.645 | 61.487 | 81.707 | 1.00 | 51.51 | A | O |
| ATOM | 265 | N | THR | A | 48 | 65.342 | 62.726 | 79.849 | 1.00 | 49.68 | A | N |
| ATOM | 266 | CA | THR | A | 48 | 63.977 | 63.098 | 80.183 | 1.00 | 48.06 | A | C |
| ATOM | 267 | CB | THR | A | 48 | 63.393 | 64.056 | 79.132 | 1.00 | 49.70 | A | C |
| ATOM | 268 | OG1 | THR | A | 48 | 64.257 | 65.191 | 78.991 | 1.00 | 52.34 | A | O |
| ATOM | 269 | CG2 | THR | A | 48 | 62.009 | 64.525 | 79.548 | 1.00 | 49.02 | A | C |
| ATOM | 270 | C | THR | A | 48 | 63.125 | 61.838 | 80.220 | 1.00 | 45.76 | A | C |
| ATOM | 271 | O | THR | A | 48 | 62.255 | 61.687 | 81.082 | 1.00 | 46.22 | A | O |
| ATOM | 272 | N | SER | A | 49 | 63.388 | 60.937 | 79.278 | 1.00 | 42.60 | A | N |
| ATOM | 273 | CA | SER | A | 49 | 62.655 | 59.680 | 79.188 | 1.00 | 41.98 | A | C |
| ATOM | 274 | CB | SER | A | 49 | 62.954 | 58.988 | 77.860 | 1.00 | 41.25 | A | C |
| ATOM | 275 | OG | SER | A | 49 | 62.194 | 57.797 | 77.741 | 1.00 | 39.90 | A | O |
| ATOM | 276 | C | SER | A | 49 | 63.011 | 58.742 | 80.344 | 1.00 | 41.06 | A | C |
| ATOM | 277 | O | SER | A | 49 | 62.148 | 58.047 | 80.891 | 1.00 | 42.09 | A | O |
| ATOM | 278 | N | PHE | A | 50 | 64.291 | 58.725 | 80.702 | 1.00 | 39.07 | A | N |
| ATOM | 279 | CA | PHE | A | 50 | 64.773 | 57.904 | 81.801 | 1.00 | 37.48 | A | C |
| ATOM | 280 | CB | PHE | A | 50 | 66.302 | 57.973 | 81.867 | 1.00 | 36.78 | A | C |
| ATOM | 281 | CG | PHE | A | 50 | 66.878 | 57.449 | 83.151 | 1.00 | 40.99 | A | C |
| ATOM | 282 | CD1 | PHE | A | 50 | 66.756 | 56.100 | 83.488 | 1.00 | 41.19 | A | C |
| ATOM | 283 | CD2 | PHE | A | 50 | 67.515 | 58.310 | 84.044 | 1.00 | 39.20 | A | C |
| ATOM | 284 | CE1 | PHE | A | 50 | 67.261 | 55.619 | 84.694 | 1.00 | 41.82 | A | C |
| ATOM | 285 | CE2 | PHE | A | 50 | 68.022 | 57.837 | 85.250 | 1.00 | 41.35 | A | C |
| ATOM | 286 | CZ | PHE | A | 50 | 67.895 | 56.493 | 85.577 | 1.00 | 42.67 | A | C |
| ATOM | 287 | C | PHE | A | 50 | 64.167 | 58.369 | 83.134 | 1.00 | 36.43 | A | C |
| ATOM | 288 | O | PHE | A | 50 | 63.659 | 57.556 | 83.911 | 1.00 | 35.74 | A | O |
| ATOM | 289 | N | THR | A | 51 | 64.207 | 59.675 | 83.388 | 1.00 | 34.93 | A | N |
| ATOM | 290 | CA | THR | A | 51 | 63.673 | 60.212 | 84.637 | 1.00 | 35.22 | A | C |
| ATOM | 291 | CB | THR | A | 51 | 63.996 | 61.735 | 84.811 | 1.00 | 36.33 | A | C |
| ATOM | 292 | OG1 | THR | A | 51 | 63.323 | 62.501 | 83.804 | 1.00 | 40.59 | A | O |
| ATOM | 293 | CG2 | THR | A | 51 | 65.484 | 61.985 | 84.684 | 1.00 | 36.69 | A | C |
| ATOM | 294 | C | THR | A | 51 | 62.169 | 60.004 | 84.717 | 1.00 | 33.76 | A | C |
| ATOM | 295 | O | THR | A | 51 | 61.628 | 59.769 | 85.795 | 1.00 | 34.22 | A | O |
| ATOM | 296 | N | PHE | A | 52 | 61.489 | 60.101 | 83.581 | 1.00 | 32.78 | A | N |
| ATOM | 297 | CA | PHE | A | 52 | 60.049 | 59.897 | 83.567 | 1.00 | 32.82 | A | C |
| ATOM | 298 | CB | PHE | A | 52 | 59.447 | 60.350 | 82.232 | 1.00 | 32.97 | A | C |
| ATOM | 299 | CG | PHE | A | 52 | 57.986 | 59.992 | 82.072 | 1.00 | 33.47 | A | C |
| ATOM | 300 | CD1 | PHE | A | 52 | 57.006 | 60.664 | 82.795 | 1.00 | 33.70 | A | C |
| ATOM | 301 | CD2 | PHE | A | 52 | 57.595 | 58.957 | 81.229 | 1.00 | 32.91 | A | C |
| ATOM | 302 | CE1 | PHE | A | 52 | 55.656 | 60.311 | 82.683 | 1.00 | 33.31 | A | C |
| ATOM | 303 | CE2 | PHE | A | 52 | 56.243 | 58.597 | 81.109 | 1.00 | 33.38 | A | C |
| ATOM | 304 | CZ | PHE | A | 52 | 55.274 | 59.278 | 81.840 | 1.00 | 33.93 | A | C |
| ATOM | 305 | C | PHE | A | 52 | 59.724 | 58.412 | 83.780 | 1.00 | 33.47 | A | C |
| ATOM | 306 | O | PHE | A | 52 | 58.897 | 58.064 | 84.631 | 1.00 | 32.75 | A | O |
| ATOM | 307 | N | LEU | A | 53 | 60.379 | 57.544 | 83.008 | 1.00 | 32.97 | A | N |
| ATOM | 308 | CA | LEU | A | 53 | 60.130 | 56.105 | 83.093 | 1.00 | 35.94 | A | C |
| ATOM | 309 | CB | LEU | A | 53 | 60.808 | 55.376 | 81.918 | 1.00 | 33.06 | A | C |
| ATOM | 310 | CG | LEU | A | 53 | 60.187 | 55.594 | 80.527 | 1.00 | 32.77 | A | C |
| ATOM | 311 | CD1 | LEU | A | 53 | 61.008 | 54.870 | 79.480 | 1.00 | 31.90 | A | C |
| ATOM | 312 | CD2 | LEU | A | 53 | 58.745 | 55.090 | 80.500 | 1.00 | 30.76 | A | C |
| ATOM | 313 | C | LEU | A | 53 | 60.525 | 55.469 | 84.428 | 1.00 | 38.03 | A | C |
| ATOM | 314 | O | LEU | A | 53 | 59.945 | 54.458 | 84.824 | 1.00 | 37.97 | A | O |
| ATOM | 315 | N | ARG | A | 54 | 61.498 | 56.061 | 85.121 | 1.00 | 41.24 | A | N |
| ATOM | 316 | CA | ARG | A | 54 | 61.934 | 55.550 | 86.428 | 1.00 | 42.92 | A | C |
| ATOM | 317 | CB | ARG | A | 54 | 63.051 | 56.397 | 87.025 | 1.00 | 46.70 | A | C |
| ATOM | 318 | CG | ARG | A | 54 | 64.440 | 56.044 | 86.610 | 1.00 | 52.00 | A | C |
| ATOM | 319 | CD | ARG | A | 54 | 65.415 | 56.722 | 87.561 | 1.00 | 56.30 | A | C |
| ATOM | 320 | NE | ARG | A | 54 | 64.998 | 58.075 | 87.919 | 1.00 | 56.95 | A | N |
| ATOM | 321 | CZ | ARG | A | 54 | 65.831 | 59.008 | 88.366 | 1.00 | 58.34 | A | C |
| ATOM | 322 | NH1 | ARG | A | 54 | 67.122 | 58.727 | 88.499 | 1.00 | 58.54 | A | N |
| ATOM | 323 | NH2 | ARG | A | 54 | 65.382 | 60.216 | 88.684 | 1.00 | 59.30 | A | N |
| ATOM | 324 | C | ARG | A | 54 | 60.799 | 55.598 | 87.426 | 1.00 | 41.97 | A | C |
| ATOM | 325 | O | ARG | A | 54 | 60.768 | 54.828 | 88.378 | 1.00 | 43.41 | A | O |
| ATOM | 326 | N | GLN | A | 55 | 59.883 | 56.532 | 87.223 | 1.00 | 40.44 | A | N |
| ATOM | 327 | CA | GLN | A | 55 | 58.761 | 56.686 | 88.128 | 1.00 | 41.07 | A | C |
| ATOM | 328 | CB | GLN | A | 55 | 58.501 | 58.180 | 88.368 | 1.00 | 42.98 | A | C |
| ATOM | 329 | CG | GLN | A | 55 | 57.502 | 58.487 | 89.464 | 1.00 | 46.30 | A | C |
| ATOM | 330 | CD | GLN | A | 55 | 57.990 | 58.041 | 90.822 | 1.00 | 50.03 | A | C |
| ATOM | 331 | OE1 | GLN | A | 55 | 59.202 | 57.935 | 91.055 | 1.00 | 53.08 | A | O |
| ATOM | 332 | NE2 | GLN | A | 55 | 57.055 | 57.792 | 91.737 | 1.00 | 49.70 | A | N |
| ATOM | 333 | C | GLN | A | 55 | 57.499 | 56.039 | 87.585 | 1.00 | 39.19 | A | C |
| ATOM | 334 | O | GLN | A | 55 | 56.797 | 55.326 | 88.302 | 1.00 | 39.52 | A | O |
| ATOM | 335 | N | GLU | A | 56 | 57.231 | 56.284 | 86.307 | 1.00 | 36.98 | A | N |
| ATOM | 336 | CA | GLU | A | 56 | 56.023 | 55.787 | 85.665 | 1.00 | 36.02 | A | C |
| ATOM | 337 | CB | GLU | A | 56 | 55.895 | 56.387 | 84.265 | 1.00 | 34.84 | A | C |
| ATOM | 338 | CG | GLU | A | 56 | 54.509 | 56.262 | 83.664 | 1.00 | 33.38 | A | C |
| ATOM | 339 | CD | GLU | A | 56 | 53.422 | 56.821 | 84.570 | 1.00 | 34.12 | A | C |
| ATOM | 340 | OE1 | GLU | A | 56 | 53.722 | 57.697 | 85.411 | 1.00 | 36.03 | A | O |
| ATOM | 341 | OE2 | GLU | A | 56 | 52.256 | 56.398 | 84.435 | 1.00 | 33.73 | A | O |
| ATOM | 342 | C | GLU | A | 56 | 55.892 | 54.278 | 85.591 | 1.00 | 33.17 | A | C |
| ATOM | 343 | O | GLU | A | 56 | 54.825 | 53.744 | 85.892 | 1.00 | 32.25 | A | O |
| ATOM | 344 | N | LEU | A | 57 | 56.955 | 53.588 | 85.187 | 1.00 | 30.49 | A | N |
| ATOM | 345 | CA | LEU | A | 57 | 56.891 | 52.133 | 85.105 | 1.00 | 30.89 | A | C |
| ATOM | 346 | CB | LEU | A | 57 | 58.188 | 51.568 | 84.524 | 1.00 | 30.81 | A | C |
| ATOM | 347 | CG | LEU | A | 57 | 58.306 | 51.916 | 83.036 | 1.00 | 30.96 | A | C |
| ATOM | 348 | CD1 | LEU | A | 57 | 59.622 | 51.432 | 82.475 | 1.00 | 31.64 | A | C |
| ATOM | 349 | CD2 | LEU | A | 57 | 57.156 | 51.287 | 82.278 | 1.00 | 31.68 | A | C |
| ATOM | 350 | C | LEU | A | 57 | 56.560 | 51.500 | 86.463 | 1.00 | 30.83 | A | C |
| ATOM | 351 | O | LEU | A | 57 | 55.705 | 50.621 | 86.543 | 1.00 | 32.12 | A | O |
| ATOM | 352 | N | PRO | A | 58 | 57.227 | 51.938 | 87.546 | 1.00 | 30.46 | A | N |
| ATOM | 353 | CD | PRO | A | 58 | 58.489 | 52.700 | 87.572 | 1.00 | 29.97 | A | C |
| ATOM | 354 | CA | PRO | A | 58 | 56.936 | 51.371 | 88.868 | 1.00 | 30.18 | A | C |
| ATOM | 355 | CB | PRO | A | 58 | 57.931 | 52.085 | 89.777 | 1.00 | 30.25 | A | C |
| ATOM | 356 | CG | PRO | A | 58 | 59.119 | 52.240 | 88.872 | 1.00 | 31.06 | A | C |
| ATOM | 357 | C | PRO | A | 58 | 55.487 | 51.643 | 89.260 | 1.00 | 30.93 | A | C |
| ATOM | 358 | O | PRO | A | 58 | 54.849 | 50.810 | 89.908 | 1.00 | 34.00 | A | O |
| ATOM | 359 | N | VAL | A | 59 | 54.971 | 52.809 | 88.871 | 1.00 | 29.89 | A | N |
| ATOM | 360 | CA | VAL | A | 59 | 53.583 | 53.168 | 89.173 | 1.00 | 28.24 | A | C |
| ATOM | 361 | CB | VAL | A | 59 | 53.251 | 54.617 | 88.715 | 1.00 | 29.45 | A | C |
| ATOM | 362 | CG1 | VAL | A | 59 | 51.758 | 54.914 | 88.941 | 1.00 | 27.52 | A | C |
| ATOM | 363 | CG2 | VAL | A | 59 | 54.114 | 55.621 | 89.487 | 1.00 | 28.36 | A | C |
| ATOM | 364 | C | VAL | A | 59 | 52.624 | 52.198 | 88.478 | 1.00 | 29.83 | A | C |
| ATOM | 365 | O | VAL | A | 59 | 51.730 | 51.630 | 89.113 | 1.00 | 31.51 | A | O |
| ATOM | 366 | N | ARG | A | 60 | 52.819 | 52.002 | 87.177 | 1.00 | 30.47 | A | N |
| ATOM | 367 | CA | ARG | A | 60 | 51.965 | 51.100 | 86.401 | 1.00 | 31.23 | A | C |
| ATOM | 368 | CB | ARG | A | 60 | 52.276 | 51.219 | 84.908 | 1.00 | 26.58 | A | C |
| ATOM | 369 | CG | ARG | A | 60 | 52.037 | 52.626 | 84.418 | 1.00 | 26.98 | A | C |
| ATOM | 370 | CD | ARG | A | 60 | 51.874 | 52.742 | 82.919 | 1.00 | 27.36 | A | C |
| ATOM | 371 | NE | ARG | A | 60 | 51.765 | 54.146 | 82.534 | 1.00 | 25.00 | A | N |
| ATOM | 372 | CZ | ARG | A | 60 | 51.547 | 54.580 | 81.296 | 1.00 | 25.41 | A | C |
| ATOM | 373 | NH1 | ARG | A | 60 | 51.400 | 53.721 | 80.287 | 1.00 | 25.13 | A | N |
| ATOM | 374 | NH2 | ARG | A | 60 | 51.502 | 55.883 | 81.064 | 1.00 | 22.39 | A | N |
| ATOM | 375 | C | ARG | A | 60 | 52.094 | 49.659 | 86.850 | 1.00 | 31.50 | A | C |
| ATOM | 376 | O | ARG | A | 60 | 51.089 | 48.961 | 86.982 | 1.00 | 33.41 | A | O |
| ATOM | 377 | N | LEU | A | 61 | 53.326 | 49.217 | 87.084 | 1.00 | 32.22 | A | N |
| ATOM | 378 | CA | LEU | A | 61 | 53.569 | 47.854 | 87.551 | 1.00 | 32.19 | A | C |
| ATOM | 379 | CB | LEU | A | 61 | 55.064 | 47.626 | 87.733 | 1.00 | 32.66 | A | C |
| ATOM | 380 | CG | LEU | A | 61 | 55.872 | 47.559 | 86.437 | 1.00 | 34.91 | A | C |
| ATOM | 381 | CD1 | LEU | A | 61 | 57.351 | 47.590 | 86.767 | 1.00 | 33.82 | A | C |
| ATOM | 382 | CD2 | LEU | A | 61 | 55.512 | 46.294 | 85.664 | 1.00 | 32.22 | A | C |
| ATOM | 383 | C | LEU | A | 61 | 52.858 | 47.615 | 88.882 | 1.00 | 32.67 | A | C |
| ATOM | 384 | O | LEU | A | 61 | 52.092 | 46.662 | 89.026 | 1.00 | 32.82 | A | O |
| ATOM | 385 | N | ALA | A | 62 | 53.112 | 48.498 | 89.844 | 1.00 | 33.49 | A | N |
| ATOM | 386 | CA | ALA | A | 62 | 52.513 | 48.402 | 91.166 | 1.00 | 35.00 | A | C |
| ATOM | 387 | CB | ALA | A | 62 | 53.018 | 49.543 | 92.044 | 1.00 | 33.60 | A | C |
| ATOM | 388 | C | ALA | A | 62 | 50.989 | 48.409 | 91.126 | 1.00 | 34.70 | A | C |
| ATOM | 389 | O | ALA | A | 62 | 50.347 | 47.619 | 91.818 | 1.00 | 35.50 | A | O |
| ATOM | 390 | N | ASN | A | 63 | 50.411 | 49.303 | 90.326 | 1.00 | 35.91 | A | N |
| ATOM | 391 | CA | ASN | A | 63 | 48.952 | 49.391 | 90.230 | 1.00 | 37.56 | A | C |
| ATOM | 392 | CB | ASN | A | 63 | 48.548 | 50.523 | 89.263 | 1.00 | 38.87 | A | C |
| ATOM | 393 | CG | ASN | A | 63 | 48.189 | 51.823 | 89.998 | 1.00 | 39.94 | A | C |
| ATOM | 394 | OD1 | ASN | A | 63 | 47.065 | 51.996 | 90.470 | 1.00 | 41.34 | A | O |
| ATOM | 395 | ND2 | ASN | A | 63 | 49.157 | 52.721 | 90.118 | 1.00 | 37.41 | A | N |
| ATOM | 396 | C | ASN | A | 63 | 48.308 | 48.063 | 89.816 | 1.00 | 37.15 | A | C |
| ATOM | 397 | O | ASN | A | 63 | 47.360 | 47.590 | 90.448 | 1.00 | 36.14 | A | O |
| ATOM | 398 | N | ILE | A | 64 | 48.829 | 47.435 | 88.772 | 1.00 | 36.63 | A | N |
| ATOM | 399 | CA | ILE | A | 64 | 48.237 | 46.182 | 88.359 | 1.00 | 38.23 | A | C |
| ATOM | 400 | CB | ILE | A | 64 | 48.639 | 45.818 | 86.910 | 1.00 | 37.96 | A | C |
| ATOM | 401 | CG2 | ILE | A | 64 | 50.050 | 45.274 | 86.861 | 1.00 | 34.75 | A | C |
| ATOM | 402 | CG1 | ILE | A | 64 | 47.619 | 44.821 | 86.347 | 1.00 | 39.27 | A | C |
| ATOM | 403 | CD1 | ILE | A | 64 | 46.177 | 45.340 | 86.378 | 1.00 | 36.43 | A | C |
| ATOM | 404 | C | ILE | A | 64 | 48.588 | 45.050 | 89.331 | 1.00 | 38.82 | A | C |
| ATOM | 405 | O | ILE | A | 64 | 47.811 | 44.108 | 89.501 | 1.00 | 39.20 | A | O |
| ATOM | 406 | N | MET | A | 65 | 49.742 | 45.150 | 89.985 | 1.00 | 37.52 | A | N |
| ATOM | 407 | CA | MET | A | 65 | 50.137 | 44.131 | 90.947 | 1.00 | 38.91 | A | C |
| ATOM | 408 | CB | MET | A | 65 | 51.591 | 44.321 | 91.359 | 1.00 | 38.09 | A | C |
| ATOM | 409 | CG | MET | A | 65 | 52.573 | 43.983 | 90.271 | 1.00 | 39.69 | A | C |
| ATOM | 410 | SD | MET | A | 65 | 54.234 | 44.570 | 90.626 | 1.00 | 40.56 | A | S |
| ATOM | 411 | CE | MET | A | 65 | 54.811 | 43.292 | 91.654 | 1.00 | 40.40 | A | C |
| ATOM | 412 | C | MET | A | 65 | 49.245 | 44.181 | 92.183 | 1.00 | 40.75 | A | C |
| ATOM | 413 | O | MET | A | 65 | 48.915 | 43.138 | 92.755 | 1.00 | 40.75 | A | O |
| ATOM | 414 | N | LYS | A | 66 | 48.862 | 45.392 | 92.591 | 1.00 | 42.36 | A | N |
| ATOM | 415 | CA | LYS | A | 66 | 48.003 | 45.570 | 93.755 | 1.00 | 44.67 | A | C |
| ATOM | 416 | CB | LYS | A | 66 | 47.814 | 47.067 | 94.052 | 1.00 | 47.56 | A | C |
| ATOM | 417 | CG | LYS | A | 66 | 46.986 | 47.403 | 95.307 | 1.00 | 50.59 | A | C |
| ATOM | 418 | CD | LYS | A | 66 | 47.741 | 47.110 | 96.603 | 1.00 | 55.26 | A | C |
| ATOM | 419 | CE | LYS | A | 66 | 46.926 | 47.498 | 97.842 | 1.00 | 56.68 | A | C |
| ATOM | 420 | NZ | LYS | A | 66 | 46.728 | 48.972 | 97.959 | 1.00 | 59.14 | A | N |
| ATOM | 421 | C | LYS | A | 66 | 46.660 | 44.915 | 93.456 | 1.00 | 44.69 | A | C |
| ATOM | 422 | O | LYS | A | 66 | 46.049 | 44.300 | 94.324 | 1.00 | 46.16 | A | O |
| ATOM | 423 | N | GLU | A | 67 | 46.213 | 45.028 | 92.212 | 1.00 | 45.37 | A | N |
| ATOM | 424 | CA | GLU | A | 67 | 44.937 | 44.453 | 91.830 | 1.00 | 46.58 | A | C |
| ATOM | 425 | CB | GLU | A | 67 | 44.481 | 45.038 | 90.498 | 1.00 | 47.73 | A | C |
| ATOM | 426 | CG | GLU | A | 67 | 42.987 | 44.985 | 90.309 | 1.00 | 50.74 | A | C |
| ATOM | 427 | CD | GLU | A | 67 | 42.525 | 45.810 | 89.132 | 1.00 | 52.29 | A | C |
| ATOM | 428 | OE1 | GLU | A | 67 | 42.926 | 46.992 | 89.033 | 1.00 | 53.67 | A | O |
| ATOM | 429 | OE2 | GLU | A | 67 | 41.751 | 45.275 | 88.312 | 1.00 | 54.20 | A | O |
| ATOM | 430 | C | GLU | A | 67 | 45.009 | 42.928 | 91.749 | 1.00 | 47.13 | A | C |
| ATOM | 431 | O | GLU | A | 67 | 44.061 | 42.236 | 92.130 | 1.00 | 47.77 | A | O |
| ATOM | 432 | N | ILE | A | 68 | 46.132 | 42.410 | 91.259 | 1.00 | 45.06 | A | N |
| ATOM | 433 | CA | ILE | A | 68 | 46.317 | 40.972 | 91.153 | 1.00 | 44.19 | A | C |
| ATOM | 434 | CB | ILE | A | 68 | 47.678 | 40.629 | 90.526 | 1.00 | 42.32 | A | C |
| ATOM | 435 | CG2 | ILE | A | 68 | 48.022 | 39.176 | 90.776 | 1.00 | 39.36 | A | C |
| ATOM | 436 | CG1 | ILE | A | 68 | 47.638 | 40.924 | 89.028 | 1.00 | 38.16 | A | C |
| ATOM | 437 | CD1 | ILE | A | 68 | 48.957 | 40.753 | 88.342 | 1.00 | 36.82 | A | C |
| ATOM | 438 | C | ILE | A | 68 | 46.220 | 40.329 | 92.530 | 1.00 | 46.99 | A | C |
| ATOM | 439 | O | ILE | A | 68 | 45.583 | 39.280 | 92.686 | 1.00 | 48.11 | A | O |
| ATOM | 440 | N | ASN | A | 69 | 46.842 | 40.963 | 93.526 | 1.00 | 48.78 | A | N |
| ATOM | 441 | CA | ASN | A | 69 | 46.803 | 40.452 | 94.897 | 1.00 | 50.29 | A | C |
| ATOM | 442 | CB | ASN | A | 69 | 47.781 | 41.197 | 95.803 | 1.00 | 51.31 | A | C |
| ATOM | 443 | CG | ASN | A | 69 | 49.220 | 40.895 | 95.483 | 1.00 | 54.32 | A | C |
| ATOM | 444 | OD1 | ASN | A | 69 | 49.553 | 39.846 | 94.928 | 1.00 | 56.45 | A | O |
| ATOM | 445 | ND2 | ASN | A | 69 | 50.096 | 41.821 | 95.850 | 1.00 | 55.72 | A | N |
| ATOM | 446 | C | ASN | A | 69 | 45.411 | 40.557 | 95.510 | 1.00 | 49.98 | A | C |
| ATOM | 447 | O | ASN | A | 69 | 45.189 | 40.091 | 96.623 | 1.00 | 52.29 | A | O |
| ATOM | 448 | N | LEU | A | 70 | 44.474 | 41.159 | 94.787 | 1.00 | 49.99 | A | N |
| ATOM | 449 | CA | LEU | A | 70 | 43.117 | 41.304 | 95.291 | 1.00 | 50.62 | A | C |
| ATOM | 450 | CB | LEU | A | 70 | 42.542 | 42.670 | 94.926 | 1.00 | 52.47 | A | C |
| ATOM | 451 | CG | LEU | A | 70 | 42.359 | 43.613 | 96.108 | 1.00 | 54.94 | A | C |
| ATOM | 452 | CD1 | LEU | A | 70 | 43.655 | 43.644 | 96.893 | 1.00 | 55.86 | A | C |
| ATOM | 453 | CD2 | LEU | A | 70 | 41.967 | 45.001 | 95.628 | 1.00 | 55.12 | A | C |
| ATOM | 454 | C | LEU | A | 70 | 42.221 | 40.213 | 94.760 | 1.00 | 49.86 | A | C |
| ATOM | 455 | O | LEU | A | 70 | 41.012 | 40.181 | 95.027 | 1.00 | 50.89 | A | O |
| ATOM | 456 | N | LEU | A | 71 | 42.806 | 39.335 | 93.971 | 1.00 | 47.16 | A | N |
| ATOM | 457 | CA | LEU | A | 71 | 42.068 | 38.203 | 93.465 | 1.00 | 46.41 | A | C |
| ATOM | 458 | CB | LEU | A | 71 | 42.871 | 37.537 | 92.310 | 1.00 | 45.77 | A | C |
| ATOM | 459 | CG | LEU | A | 71 | 42.883 | 38.298 | 90.962 | 1.00 | 45.51 | A | C |
| ATOM | 460 | CD1 | LEU | A | 71 | 44.128 | 37.934 | 90.164 | 1.00 | 44.86 | A | C |
| ATOM | 461 | CD2 | LEU | A | 71 | 41.628 | 37.992 | 90.115 | 1.00 | 43.43 | A | C |
| ATOM | 462 | C | LEU | A | 71 | 41.846 | 37.133 | 94.541 | 1.00 | 44.48 | A | C |
| ATOM | 463 | O | LEU | A | 71 | 42.636 | 36.985 | 95.477 | 1.00 | 43.73 | A | O |
| ATOM | 464 | N | PRO | A | 72 | 40.774 | 36.338 | 94.387 | 1.00 | 42.16 | A | N |
| ATOM | 465 | CD | PRO | A | 72 | 39.775 | 36.377 | 93.296 | 1.00 | 41.59 | A | C |
| ATOM | 466 | CA | PRO | A | 72 | 40.456 | 35.244 | 95.318 | 1.00 | 42.25 | A | C |
| ATOM | 467 | CB | PRO | A | 72 | 39.317 | 34.505 | 94.595 | 1.00 | 41.85 | A | C |
| ATOM | 468 | CG | PRO | A | 72 | 38.616 | 35.634 | 93.891 | 1.00 | 41.62 | A | C |
| ATOM | 469 | C | PRO | A | 72 | 41.689 | 34.328 | 95.488 | 1.00 | 42.19 | A | C |
| ATOM | 470 | O | PRO | A | 72 | 42.496 | 34.184 | 94.579 | 1.00 | 42.36 | A | O |
| ATOM | 471 | N | ASP | A | 73 | 41.830 | 33.714 | 96.653 | 1.00 | 44.07 | A | N |
| ATOM | 472 | CA | ASP | A | 73 | 42.988 | 32.873 | 96.880 | 1.00 | 44.56 | A | C |
| ATOM | 473 | CB | ASP | A | 73 | 43.059 | 32.421 | 98.334 | 1.00 | 49.47 | A | C |
| ATOM | 474 | CG | ASP | A | 73 | 44.297 | 31.569 | 98.621 | 1.00 | 53.86 | A | C |
| ATOM | 475 | OD1 | ASP | A | 73 | 44.157 | 30.348 | 98.816 | 1.00 | 54.48 | A | O |
| ATOM | 476 | OD2 | ASP | A | 73 | 45.412 | 32.122 | 98.666 | 1.00 | 56.23 | A | O |
| ATOM | 477 | C | ASP | A | 73 | 43.002 | 31.664 | 95.960 | 1.00 | 41.46 | A | C |
| ATOM | 478 | O | ASP | A | 73 | 44.072 | 31.189 | 95.583 | 1.00 | 40.02 | A | O |
| ATOM | 479 | N | ARG | A | 74 | 41.824 | 31.156 | 95.596 | 1.00 | 40.05 | A | N |
| ATOM | 480 | CA | ARG | A | 74 | 41.750 | 29.993 | 94.704 | 1.00 | 39.97 | A | C |
| ATOM | 481 | CB | ARG | A | 74 | 40.314 | 29.441 | 94.616 | 1.00 | 37.97 | A | C |
| ATOM | 482 | CG | ARG | A | 74 | 39.285 | 30.427 | 94.118 | 1.00 | 37.75 | A | C |
| ATOM | 483 | CD | ARG | A | 74 | 37.932 | 29.745 | 94.098 | 1.00 | 37.66 | A | C |
| ATOM | 484 | NE | ARG | A | 74 | 36.866 | 30.547 | 93.499 | 1.00 | 38.25 | A | N |
| ATOM | 485 | CZ | ARG | A | 74 | 36.411 | 31.691 | 93.999 | 1.00 | 37.26 | A | C |
| ATOM | 486 | NH1 | ARG | A | 74 | 36.930 | 32.183 | 95.115 | 1.00 | 36.11 | A | N |
| ATOM | 487 | NH2 | ARG | A | 74 | 35.424 | 32.336 | 93.386 | 1.00 | 35.20 | A | N |
| ATOM | 488 | C | ARG | A | 74 | 42.267 | 30.337 | 93.319 | 1.00 | 39.55 | A | C |
| ATOM | 489 | O | ARG | A | 74 | 42.571 | 29.449 | 92.514 | 1.00 | 40.65 | A | O |
| ATOM | 490 | N | VAL | A | 75 | 42.365 | 31.631 | 93.044 | 1.00 | 38.90 | A | N |
| ATOM | 491 | CA | VAL | A | 75 | 42.875 | 32.077 | 91.754 | 1.00 | 37.95 | A | C |
| ATOM | 492 | CB | VAL | A | 75 | 42.097 | 33.308 | 91.236 | 1.00 | 39.31 | A | C |
| ATOM | 493 | CG1 | VAL | A | 75 | 42.669 | 33.783 | 89.914 | 1.00 | 36.04 | A | C |
| ATOM | 494 | CG2 | VAL | A | 75 | 40.612 | 32.967 | 91.072 | 1.00 | 37.92 | A | C |
| ATOM | 495 | C | VAL | A | 75 | 44.344 | 32.459 | 91.905 | 1.00 | 38.82 | A | C |
| ATOM | 496 | O | VAL | A | 75 | 45.185 | 32.102 | 91.079 | 1.00 | 38.55 | A | O |
| ATOM | 497 | N | LEU | A | 76 | 44.640 | 33.175 | 92.984 | 1.00 | 40.40 | A | N |
| ATOM | 498 | CA | LEU | A | 76 | 45.991 | 33.628 | 93.294 | 1.00 | 43.75 | A | C |
| ATOM | 499 | CB | LEU | A | 76 | 45.990 | 34.414 | 94.608 | 1.00 | 46.01 | A | C |
| ATOM | 500 | CG | LEU | A | 76 | 45.977 | 35.928 | 94.533 | 1.00 | 48.48 | A | C |
| ATOM | 501 | CD1 | LEU | A | 76 | 45.729 | 36.480 | 95.922 | 1.00 | 49.52 | A | C |
| ATOM | 502 | CD2 | LEU | A | 76 | 47.302 | 36.405 | 93.967 | 1.00 | 46.56 | A | C |
| ATOM | 503 | C | LEU | A | 76 | 47.007 | 32.500 | 93.434 | 1.00 | 44.55 | A | C |
| ATOM | 504 | O | LEU | A | 76 | 48.099 | 32.577 | 92.879 | 1.00 | 46.85 | A | O |
| ATOM | 505 | N | SER | A | 77 | 46.649 | 31.458 | 94.176 | 1.00 | 43.24 | A | N |
| ATOM | 506 | CA | SER | A | 77 | 47.575 | 30.362 | 94.426 | 1.00 | 44.52 | A | C |
| ATOM | 507 | CB | SER | A | 77 | 47.071 | 29.512 | 95.583 | 1.00 | 43.67 | A | C |
| ATOM | 508 | OG | SER | A | 77 | 45.832 | 28.900 | 95.279 | 1.00 | 45.35 | A | O |
| ATOM | 509 | C | SER | A | 77 | 47.841 | 29.459 | 93.226 | 1.00 | 44.00 | A | C |
| ATOM | 510 | O | SER | A | 77 | 48.711 | 28.561 | 93.297 | 1.00 | 46.31 | A | O |
| ATOM | 511 | N | THR | A | 78 | 47.121 | 29.672 | 92.128 | 1.00 | 41.08 | A | N |
| ATOM | 512 | CA | THR | A | 78 | 47.348 | 28.832 | 90.968 | 1.00 | 40.61 | A | C |
| ATOM | 513 | CB | THR | A | 78 | 46.348 | 29.161 | 89.824 | 1.00 | 42.41 | A | C |
| ATOM | 514 | OG1 | THR | A | 78 | 46.580 | 30.498 | 89.355 | 1.00 | 44.74 | A | O |
| ATOM | 515 | CG2 | THR | A | 78 | 44.933 | 29.002 | 90.294 | 1.00 | 43.88 | A | C |
| ATOM | 516 | C | THR | A | 78 | 48.759 | 29.069 | 90.456 | 1.00 | 39.68 | A | C |
| ATOM | 517 | O | THR | A | 78 | 49.266 | 30.175 | 90.546 | 1.00 | 38.39 | A | O |
| ATOM | 518 | N | PRO | A | 79 | 49.408 | 28.036 | 89.904 | 1.00 | 38.47 | A | N |
| ATOM | 519 | CD | PRO | A | 79 | 48.965 | 26.640 | 89.736 | 1.00 | 37.15 | A | C |
| ATOM | 520 | CA | PRO | A | 79 | 50.774 | 28.220 | 89.396 | 1.00 | 36.45 | A | C |
| ATOM | 521 | CB | PRO | A | 79 | 51.109 | 26.841 | 88.805 | 1.00 | 34.49 | A | C |
| ATOM | 522 | CG | PRO | A | 79 | 50.276 | 25.914 | 89.634 | 1.00 | 37.71 | A | C |
| ATOM | 523 | C | PRO | A | 79 | 50.981 | 29.359 | 88.391 | 1.00 | 36.17 | A | C |
| ATOM | 524 | O | PRO | A | 79 | 51.962 | 30.084 | 88.444 | 1.00 | 35.53 | A | O |
| ATOM | 525 | N | SER | A | 80 | 50.011 | 29.535 | 87.515 | 1.00 | 33.53 | A | N |
| ATOM | 526 | CA | SER | A | 80 | 50.103 | 30.523 | 86.458 | 1.00 | 32.31 | A | C |
| ATOM | 527 | CB | SER | A | 80 | 48.955 | 30.238 | 85.467 | 1.00 | 30.39 | A | C |
| ATOM | 528 | OG | SER | A | 80 | 47.683 | 30.525 | 86.102 | 1.00 | 37.05 | A | O |
| ATOM | 529 | C | SER | A | 80 | 50.127 | 31.982 | 86.982 | 1.00 | 32.52 | A | C |
| ATOM | 530 | O | SER | A | 80 | 51.061 | 32.756 | 86.683 | 1.00 | 31.17 | A | O |
| ATOM | 531 | N | VAL | A | 81 | 49.153 | 32.327 | 87.820 | 1.00 | 32.49 | A | N |
| ATOM | 532 | CA | VAL | A | 81 | 49.076 | 33.657 | 88.383 | 1.00 | 33.35 | A | C |
| ATOM | 533 | CB | VAL | A | 81 | 47.795 | 33.836 | 89.192 | 1.00 | 33.25 | A | C |
| ATOM | 534 | CG1 | VAL | A | 81 | 47.747 | 35.249 | 89.773 | 1.00 | 31.05 | A | C |
| ATOM | 535 | CG2 | VAL | A | 81 | 46.598 | 33.590 | 88.281 | 1.00 | 30.13 | A | C |
| ATOM | 536 | C | VAL | A | 81 | 50.283 | 33.936 | 89.261 | 1.00 | 34.95 | A | C |
| ATOM | 537 | O | VAL | A | 81 | 50.806 | 35.047 | 89.242 | 1.00 | 35.56 | A | O |
| ATOM | 538 | N | GLN | A | 82 | 50.739 | 32.953 | 90.030 | 1.00 | 37.43 | A | N |
| ATOM | 539 | CA | GLN | A | 82 | 51.911 | 33.201 | 90.865 | 1.00 | 39.47 | A | C |
| ATOM | 540 | CB | GLN | A | 82 | 52.112 | 32.066 | 91.888 | 1.00 | 44.11 | A | C |
| ATOM | 541 | CG | GLN | A | 82 | 51.867 | 32.526 | 93.335 | 1.00 | 54.94 | A | C |
| ATOM | 542 | CD | GLN | A | 82 | 52.346 | 31.531 | 94.390 | 1.00 | 63.22 | A | C |
| ATOM | 543 | OE1 | GLN | A | 82 | 53.542 | 31.223 | 94.485 | 1.00 | 67.91 | A | O |
| ATOM | 544 | NE2 | GLN | A | 82 | 51.411 | 31.033 | 95.199 | 1.00 | 65.03 | A | N |
| ATOM | 545 | C | GLN | A | 82 | 53.167 | 33.387 | 89.997 | 1.00 | 36.80 | A | C |
| ATOM | 546 | O | GLN | A | 82 | 54.055 | 34.174 | 90.331 | 1.00 | 36.43 | A | O |
| ATOM | 547 | N | LEU | A | 83 | 53.232 | 32.685 | 88.871 | 1.00 | 32.98 | A | N |
| ATOM | 548 | CA | LEU | A | 83 | 54.389 | 32.801 | 88.006 | 1.00 | 33.32 | A | C |
| ATOM | 549 | CB | LEU | A | 83 | 54.354 | 31.739 | 86.904 | 1.00 | 33.13 | A | C |
| ATOM | 550 | CG | LEU | A | 83 | 55.450 | 31.907 | 85.852 | 1.00 | 33.95 | A | C |
| ATOM | 551 | CD1 | LEU | A | 83 | 56.800 | 31.735 | 86.498 | 1.00 | 32.99 | A | C |
| ATOM | 552 | CD2 | LEU | A | 83 | 55.261 | 30.897 | 84.743 | 1.00 | 31.71 | A | C |
| ATOM | 553 | C | LEU | A | 83 | 54.507 | 34.193 | 87.386 | 1.00 | 35.13 | A | C |
| ATOM | 554 | O | LEU | A | 83 | 55.573 | 34.812 | 87.475 | 1.00 | 33.61 | A | O |
| ATOM | 555 | N | VAL | A | 84 | 53.437 | 34.708 | 86.771 | 1.00 | 33.98 | A | N |
| ATOM | 556 | CA | VAL | A | 84 | 53.553 | 36.038 | 86.181 | 1.00 | 33.57 | A | C |
| ATOM | 557 | CB | VAL | A | 84 | 52.376 | 36.415 | 85.214 | 1.00 | 30.66 | A | C |
| ATOM | 558 | CG1 | VAL | A | 84 | 51.823 | 35.180 | 84.522 | 1.00 | 26.00 | A | C |
| ATOM | 559 | CG2 | VAL | A | 84 | 51.328 | 37.201 | 85.945 | 1.00 | 31.02 | A | C |
| ATOM | 560 | C | VAL | A | 84 | 53.655 | 37.086 | 87.283 | 1.00 | 34.69 | A | C |
| ATOM | 561 | O | VAL | A | 84 | 54.325 | 38.101 | 87.108 | 1.00 | 34.91 | A | O |
| ATOM | 562 | N | GLN | A | 85 | 53.004 | 36.837 | 88.417 | 1.00 | 35.14 | A | N |
| ATOM | 563 | CA | GLN | A | 85 | 53.064 | 37.781 | 89.522 | 1.00 | 37.01 | A | C |
| ATOM | 564 | CB | GLN | A | 85 | 52.181 | 37.323 | 90.691 | 1.00 | 38.78 | A | C |
| ATOM | 565 | CG | GLN | A | 85 | 52.244 | 38.210 | 91.946 | 1.00 | 46.00 | A | C |
| ATOM | 566 | CD | GLN | A | 85 | 51.686 | 39.628 | 91.732 | 1.00 | 53.77 | A | C |
| ATOM | 567 | OE1 | GLN | A | 85 | 52.197 | 40.401 | 90.910 | 1.00 | 52.86 | A | O |
| ATOM | 568 | NE2 | GLN | A | 85 | 50.635 | 39.972 | 92.481 | 1.00 | 55.53 | A | N |
| ATOM | 569 | C | GLN | A | 85 | 54.514 | 37.885 | 89.971 | 1.00 | 38.00 | A | C |
| ATOM | 570 | O | GLN | A | 85 | 54.918 | 38.890 | 90.555 | 1.00 | 39.23 | A | O |
| ATOM | 571 | N | SER | A | 86 | 55.305 | 36.850 | 89.697 | 1.00 | 37.57 | A | N |
| ATOM | 572 | CA | SER | A | 86 | 56.711 | 36.885 | 90.082 | 1.00 | 36.28 | A | C |
| ATOM | 573 | CB | SER | A | 86 | 57.279 | 35.469 | 90.301 | 1.00 | 35.48 | A | C |
| ATOM | 574 | OG | SER | A | 86 | 57.476 | 34.758 | 89.091 | 1.00 | 35.95 | A | O |
| ATOM | 575 | C | SER | A | 86 | 57.510 | 37.635 | 89.022 | 1.00 | 35.61 | A | C |
| ATOM | 576 | O | SER | A | 86 | 58.489 | 38.300 | 89.347 | 1.00 | 37.40 | A | O |
| ATOM | 577 | N | TRP | A | 87 | 57.111 | 37.534 | 87.757 | 1.00 | 33.83 | A | N |
| ATOM | 578 | CA | TRP | A | 87 | 57.814 | 38.283 | 86.714 | 1.00 | 34.17 | A | C |
| ATOM | 579 | CB | TRP | A | 87 | 57.251 | 37.993 | 85.313 | 1.00 | 33.74 | A | C |
| ATOM | 580 | CG | TRP | A | 87 | 57.522 | 36.615 | 84.806 | 1.00 | 35.50 | A | C |
| ATOM | 581 | CD2 | TRP | A | 87 | 56.789 | 35.921 | 83.792 | 1.00 | 36.46 | A | C |
| ATOM | 582 | CE2 | TRP | A | 87 | 57.396 | 34.653 | 83.628 | 1.00 | 35.44 | A | C |
| ATOM | 583 | CE3 | TRP | A | 87 | 55.677 | 36.248 | 83.001 | 1.00 | 35.38 | A | C |
| ATOM | 584 | CD1 | TRP | A | 87 | 58.521 | 35.766 | 85.206 | 1.00 | 36.37 | A | C |
| ATOM | 585 | NE1 | TRP | A | 87 | 58.449 | 34.584 | 84.501 | 1.00 | 34.59 | A | N |
| ATOM | 586 | CZ2 | TRP | A | 87 | 56.925 | 33.710 | 82.707 | 1.00 | 34.98 | A | C |
| ATOM | 587 | CZ3 | TRP | A | 87 | 55.211 | 35.312 | 82.086 | 1.00 | 34.89 | A | C |
| ATOM | 588 | CH2 | TRP | A | 87 | 55.837 | 34.057 | 81.948 | 1.00 | 35.99 | A | C |
| ATOM | 589 | C | TRP | A | 87 | 57.648 | 39.775 | 87.018 | 1.00 | 32.82 | A | C |
| ATOM | 590 | O | TRP | A | 87 | 58.603 | 40.546 | 86.958 | 1.00 | 31.38 | A | O |
| ATOM | 591 | N | TYR | A | 88 | 56.427 | 40.175 | 87.351 | 1.00 | 30.82 | A | N |
| ATOM | 592 | CA | TYR | A | 88 | 56.170 | 41.572 | 87.658 | 1.00 | 32.27 | A | C |
| ATOM | 593 | CB | TYR | A | 88 | 54.683 | 41.786 | 87.921 | 1.00 | 30.67 | A | C |
| ATOM | 594 | CG | TYR | A | 88 | 53.918 | 41.967 | 86.635 | 1.00 | 35.03 | A | C |
| ATOM | 595 | CD1 | TYR | A | 88 | 53.508 | 43.232 | 86.212 | 1.00 | 36.92 | A | C |
| ATOM | 596 | CE1 | TYR | A | 88 | 52.876 | 43.410 | 84.979 | 1.00 | 38.15 | A | C |
| ATOM | 597 | CD2 | TYR | A | 88 | 53.673 | 40.888 | 85.797 | 1.00 | 34.27 | A | C |
| ATOM | 598 | CE2 | TYR | A | 88 | 53.046 | 41.056 | 84.573 | 1.00 | 36.76 | A | C |
| ATOM | 599 | CZ | TYR | A | 88 | 52.651 | 42.312 | 84.169 | 1.00 | 36.63 | A | C |
| ATOM | 600 | OH | TYR | A | 88 | 52.030 | 42.462 | 82.957 | 1.00 | 40.37 | A | O |
| ATOM | 601 | C | TYR | A | 88 | 57.007 | 42.041 | 88.839 | 1.00 | 34.46 | A | C |
| ATOM | 602 | O | TYR | A | 88 | 57.590 | 43.126 | 88.794 | 1.00 | 33.70 | A | O |
| ATOM | 603 | N | VAL | A | 89 | 57.078 | 41.221 | 89.886 | 1.00 | 35.55 | A | N |
| ATOM | 604 | CA | VAL | A | 89 | 57.865 | 41.563 | 91.066 | 1.00 | 34.15 | A | C |
| ATOM | 605 | CB | VAL | A | 89 | 57.820 | 40.430 | 92.112 | 1.00 | 36.21 | A | C |
| ATOM | 606 | CG1 | VAL | A | 89 | 58.939 | 40.620 | 93.136 | 1.00 | 34.66 | A | C |
| ATOM | 607 | CG2 | VAL | A | 89 | 56.457 | 40.421 | 92.814 | 1.00 | 34.90 | A | C |
| ATOM | 608 | C | VAL | A | 89 | 59.308 | 41.795 | 90.639 | 1.00 | 35.71 | A | C |
| ATOM | 609 | O | VAL | A | 89 | 59.907 | 42.829 | 90.941 | 1.00 | 34.26 | A | O |
| ATOM | 610 | N | GLN | A | 90 | 59.857 | 40.832 | 89.910 | 1.00 | 36.27 | A | N |
| ATOM | 611 | CA | GLN | A | 90 | 61.229 | 40.935 | 89.447 | 1.00 | 36.52 | A | C |
| ATOM | 612 | CB | GLN | A | 90 | 61.626 | 39.676 | 88.679 | 1.00 | 38.55 | A | C |
| ATOM | 613 | CG | GLN | A | 90 | 63.131 | 39.519 | 88.534 | 1.00 | 42.19 | A | C |
| ATOM | 614 | CD | GLN | A | 90 | 63.857 | 39.705 | 89.865 | 1.00 | 45.17 | A | C |
| ATOM | 615 | OE1 | GLN | A | 90 | 63.547 | 39.036 | 90.857 | 1.00 | 47.56 | A | O |
| ATOM | 616 | NE2 | GLN | A | 90 | 64.822 | 40.622 | 89.891 | 1.00 | 41.65 | A | N |
| ATOM | 617 | C | GLN | A | 90 | 61.442 | 42.164 | 88.565 | 1.00 | 37.39 | A | C |
| ATOM | 618 | O | GLN | A | 90 | 62.446 | 42.867 | 88.692 | 1.00 | 36.63 | A | O |
| ATOM | 619 | N | SER | A | 91 | 60.506 | 42.421 | 87.658 | 1.00 | 36.89 | A | N |
| ATOM | 620 | CA | SER | A | 91 | 60.619 | 43.580 | 86.777 | 1.00 | 35.70 | A | C |
| ATOM | 621 | CB | SER | A | 91 | 59.473 | 43.590 | 85.763 | 1.00 | 36.08 | A | C |
| ATOM | 622 | OG | SER | A | 91 | 59.722 | 42.661 | 84.721 | 1.00 | 37.26 | A | O |
| ATOM | 623 | C | SER | A | 91 | 60.624 | 44.882 | 87.577 | 1.00 | 35.65 | A | C |
| ATOM | 624 | O | SER | A | 91 | 61.369 | 45.815 | 87.265 | 1.00 | 34.04 | A | O |
| ATOM | 625 | N | LEU | A | 92 | 59.785 | 44.937 | 88.607 | 1.00 | 35.53 | A | N |
| ATOM | 626 | CA | LEU | A | 92 | 59.699 | 46.109 | 89.476 | 1.00 | 36.65 | A | C |
| ATOM | 627 | CB | LEU | A | 92 | 58.606 | 45.912 | 90.535 | 1.00 | 35.11 | A | C |
| ATOM | 628 | CG | LEU | A | 92 | 58.410 | 47.012 | 91.585 | 1.00 | 38.04 | A | C |
| ATOM | 629 | CD1 | LEU | A | 92 | 57.993 | 48.297 | 90.902 | 1.00 | 36.19 | A | C |
| ATOM | 630 | CD2 | LEU | A | 92 | 57.346 | 46.588 | 92.588 | 1.00 | 37.04 | A | C |
| ATOM | 631 | C | LEU | A | 92 | 61.041 | 46.371 | 90.171 | 1.00 | 38.33 | A | C |
| ATOM | 632 | O | LEU | A | 92 | 61.528 | 47.498 | 90.154 | 1.00 | 39.32 | A | O |
| ATOM | 633 | N | LEU | A | 93 | 61.626 | 45.341 | 90.788 | 1.00 | 38.42 | A | N |
| ATOM | 634 | CA | LEU | A | 93 | 62.913 | 45.482 | 91.474 | 1.00 | 37.62 | A | C |
| ATOM | 635 | CB | LEU | A | 93 | 63.286 | 44.171 | 92.181 | 1.00 | 35.98 | A | C |
| ATOM | 636 | CG | LEU | A | 93 | 62.263 | 43.688 | 93.217 | 1.00 | 36.40 | A | C |
| ATOM | 637 | CD1 | LEU | A | 93 | 62.769 | 42.426 | 93.893 | 1.00 | 33.15 | A | C |
| ATOM | 638 | CD2 | LEU | A | 93 | 62.020 | 44.779 | 94.259 | 1.00 | 37.08 | A | C |
| ATOM | 639 | C | LEU | A | 93 | 64.007 | 45.879 | 90.478 | 1.00 | 38.35 | A | C |
| ATOM | 640 | O | LEU | A | 93 | 64.882 | 46.689 | 90.784 | 1.00 | 37.95 | A | O |
| ATOM | 641 | N | ASP | A | 94 | 63.951 | 45.309 | 89.281 | 1.00 | 39.06 | A | N |
| ATOM | 642 | CA | ASP | A | 94 | 64.919 | 45.633 | 88.238 | 1.00 | 41.11 | A | C |
| ATOM | 643 | CB | ASP | A | 94 | 64.551 | 44.924 | 86.938 | 1.00 | 42.65 | A | C |
| ATOM | 644 | CG | ASP | A | 94 | 65.092 | 43.518 | 86.863 | 1.00 | 41.73 | A | C |
| ATOM | 645 | OD1 | ASP | A | 94 | 65.251 | 42.878 | 87.919 | 1.00 | 45.20 | A | O |
| ATOM | 646 | OD2 | ASP | A | 94 | 65.345 | 43.046 | 85.737 | 1.00 | 42.46 | A | O |
| ATOM | 647 | C | ASP | A | 94 | 64.967 | 47.137 | 87.972 | 1.00 | 43.15 | A | C |
| ATOM | 648 | O | ASP | A | 94 | 66.041 | 47.712 | 87.775 | 1.00 | 43.92 | A | O |
| ATOM | 649 | N | ILE | A | 95 | 63.801 | 47.776 | 87.963 | 1.00 | 43.80 | A | N |
| ATOM | 650 | CA | ILE | A | 95 | 63.751 | 49.206 | 87.697 | 1.00 | 46.38 | A | C |
| ATOM | 651 | CB | ILE | A | 95 | 62.380 | 49.611 | 87.100 | 1.00 | 45.12 | A | C |
| ATOM | 652 | CG2 | ILE | A | 95 | 62.299 | 51.119 | 86.910 | 1.00 | 43.88 | A | C |
| ATOM | 653 | CG1 | ILE | A | 95 | 62.221 | 48.946 | 85.731 | 1.00 | 44.75 | A | C |
| ATOM | 654 | CD1 | ILE | A | 95 | 60.898 | 49.174 | 85.086 | 1.00 | 47.42 | A | C |
| ATOM | 655 | C | ILE | A | 95 | 64.091 | 50.069 | 88.906 | 1.00 | 48.07 | A | C |
| ATOM | 656 | O | ILE | A | 95 | 64.693 | 51.130 | 88.756 | 1.00 | 48.13 | A | O |
| ATOM | 657 | N | MET | A | 96 | 63.721 | 49.612 | 90.097 | 1.00 | 50.79 | A | N |
| ATOM | 658 | CA | MET | A | 96 | 64.027 | 50.341 | 91.327 | 1.00 | 54.00 | A | C |
| ATOM | 659 | CB | MET | A | 96 | 63.791 | 49.440 | 92.541 | 1.00 | 55.65 | A | C |
| ATOM | 660 | CG | MET | A | 96 | 62.413 | 48.851 | 92.617 | 1.00 | 56.61 | A | C |
| ATOM | 661 | SD | MET | A | 96 | 61.226 | 50.159 | 92.853 | 1.00 | 60.62 | A | S |
| ATOM | 662 | CE | MET | A | 96 | 61.116 | 50.176 | 94.550 | 1.00 | 56.66 | A | C |
| ATOM | 663 | C | MET | A | 96 | 65.503 | 50.752 | 91.317 | 1.00 | 54.67 | A | C |
| ATOM | 664 | O | MET | A | 96 | 65.853 | 51.893 | 91.627 | 1.00 | 55.97 | A | O |
| ATOM | 665 | N | GLU | A | 97 | 66.350 | 49.791 | 90.959 | 1.00 | 54.34 | A | N |
| ATOM | 666 | CA | GLU | A | 97 | 67.801 | 49.951 | 90.892 | 1.00 | 55.29 | A | C |
| ATOM | 667 | CB | GLU | A | 97 | 68.404 | 48.834 | 90.029 | 1.00 | 57.43 | A | C |
| ATOM | 668 | CG | GLU | A | 97 | 69.468 | 47.964 | 90.687 | 1.00 | 61.05 | A | C |
| ATOM | 669 | CD | GLU | A | 97 | 68.903 | 47.065 | 91.767 | 1.00 | 63.66 | A | C |
| ATOM | 670 | OE1 | GLU | A | 97 | 67.780 | 46.550 | 91.587 | 1.00 | 67.58 | A | O |
| ATOM | 671 | OE2 | GLU | A | 97 | 69.588 | 46.862 | 92.789 | 1.00 | 64.30 | A | O |
| ATOM | 672 | C | GLU | A | 97 | 68.276 | 51.287 | 90.336 | 1.00 | 54.67 | A | C |
| ATOM | 673 | O | GLU | A | 97 | 69.434 | 51.648 | 90.521 | 1.00 | 55.23 | A | O |
| ATOM | 674 | N | PHE | A | 98 | 67.397 | 52.016 | 89.657 | 1.00 | 54.17 | A | N |
| ATOM | 675 | CA | PHE | A | 98 | 67.772 | 53.291 | 89.050 | 1.00 | 54.14 | A | C |
| ATOM | 676 | CB | PHE | A | 98 | 67.330 | 53.302 | 87.587 | 1.00 | 50.29 | A | C |
| ATOM | 677 | CG | PHE | A | 98 | 67.914 | 52.193 | 86.772 | 1.00 | 44.44 | A | C |
| ATOM | 678 | CD1 | PHE | A | 98 | 69.007 | 52.420 | 85.949 | 1.00 | 43.46 | A | C |
| ATOM | 679 | CD2 | PHE | A | 98 | 67.372 | 50.917 | 86.826 | 1.00 | 42.02 | A | C |
| ATOM | 680 | CE1 | PHE | A | 98 | 69.554 | 51.387 | 85.184 | 1.00 | 41.28 | A | C |
| ATOM | 681 | CE2 | PHE | A | 98 | 67.912 | 49.878 | 86.067 | 1.00 | 39.44 | A | C |
| ATOM | 682 | CZ | PHE | A | 98 | 69.001 | 50.114 | 85.246 | 1.00 | 39.50 | A | C |
| ATOM | 683 | C | PHE | A | 98 | 67.233 | 54.548 | 89.725 | 1.00 | 56.36 | A | C |
| ATOM | 684 | O | PHE | A | 98 | 67.597 | 55.660 | 89.338 | 1.00 | 57.63 | A | O |
| ATOM | 685 | N | LEU | A | 99 | 66.369 | 54.392 | 90.720 | 1.00 | 58.96 | A | N |
| ATOM | 686 | CA | LEU | A | 99 | 65.804 | 55.557 | 91.384 | 1.00 | 63.14 | A | C |
| ATOM | 687 | CB | LEU | A | 99 | 64.843 | 55.118 | 92.488 | 1.00 | 62.73 | A | C |
| ATOM | 688 | CG | LEU | A | 99 | 63.547 | 54.526 | 91.928 | 1.00 | 64.15 | A | C |
| ATOM | 689 | CD1 | LEU | A | 99 | 62.700 | 53.964 | 93.061 | 1.00 | 64.48 | A | C |
| ATOM | 690 | CD2 | LEU | A | 99 | 62.788 | 55.603 | 91.150 | 1.00 | 63.56 | A | C |
| ATOM | 691 | C | LEU | A | 99 | 66.856 | 56.511 | 91.937 | 1.00 | 65.39 | A | C |
| ATOM | 692 | O | LEU | A | 99 | 66.763 | 57.721 | 91.731 | 1.00 | 65.24 | A | O |
| ATOM | 693 | N | ASP | A | 100 | 67.858 | 55.965 | 92.623 | 1.00 | 68.70 | A | N |
| ATOM | 694 | CA | ASP | A | 100 | 68.925 | 56.773 | 93.207 | 1.00 | 71.57 | A | C |
| ATOM | 695 | CB | ASP | A | 100 | 69.296 | 56.236 | 94.594 | 1.00 | 74.46 | A | C |
| ATOM | 696 | CG | ASP | A | 100 | 68.355 | 56.733 | 95.686 | 1.00 | 78.14 | A | C |
| ATOM | 697 | OD1 | ASP | A | 100 | 67.124 | 56.700 | 95.478 | 1.00 | 80.18 | A | O |
| ATOM | 698 | OD2 | ASP | A | 100 | 68.850 | 57.154 | 96.756 | 1.00 | 80.38 | A | O |
| ATOM | 699 | C | ASP | A | 100 | 70.163 | 56.834 | 92.319 | 1.00 | 71.98 | A | C |
| ATOM | 700 | O | ASP | A | 100 | 71.284 | 56.988 | 92.808 | 1.00 | 72.48 | A | O |
| ATOM | 701 | N | LYS | A | 101 | 69.947 | 56.701 | 91.013 | 1.00 | 71.77 | A | N |
| ATOM | 702 | CA | LYS | A | 101 | 71.028 | 56.771 | 90.034 | 1.00 | 71.59 | A | C |
| ATOM | 703 | CB | LYS | A | 101 | 70.905 | 55.656 | 88.993 | 1.00 | 70.80 | A | C |
| ATOM | 704 | CG | LYS | A | 101 | 71.806 | 54.455 | 89.241 | 1.00 | 71.08 | A | C |
| ATOM | 705 | CD | LYS | A | 101 | 71.568 | 53.846 | 90.616 | 1.00 | 73.16 | A | C |
| ATOM | 706 | CE | LYS | A | 101 | 72.516 | 52.687 | 90.883 | 1.00 | 73.63 | A | C |
| ATOM | 707 | NZ | LYS | A | 101 | 72.318 | 52.119 | 92.250 | 1.00 | 75.53 | A | N |
| ATOM | 708 | C | LYS | A | 101 | 70.883 | 58.120 | 89.362 | 1.00 | 72.69 | A | C |
| ATOM | 709 | O | LYS | A | 101 | 69.812 | 58.724 | 89.419 | 1.00 | 73.30 | A | O |
| ATOM | 710 | N | ASP | A | 102 | 71.943 | 58.599 | 88.721 | 1.00 | 73.13 | A | N |
| ATOM | 711 | CA | ASP | A | 102 | 71.875 | 59.904 | 88.081 | 1.00 | 73.67 | A | C |
| ATOM | 712 | CB | ASP | A | 102 | 72.847 | 60.883 | 88.757 | 1.00 | 76.23 | A | C |
| ATOM | 713 | CG | ASP | A | 102 | 72.389 | 61.304 | 90.148 | 1.00 | 79.23 | A | C |
| ATOM | 714 | OD1 | ASP | A | 102 | 71.294 | 61.898 | 90.264 | 1.00 | 80.88 | A | O |
| ATOM | 715 | OD2 | ASP | A | 102 | 73.125 | 61.041 | 91.125 | 1.00 | 80.62 | A | O |
| ATOM | 716 | C | ASP | A | 102 | 72.149 | 59.905 | 86.590 | 1.00 | 72.27 | A | C |
| ATOM | 717 | O | ASP | A | 102 | 73.084 | 59.256 | 86.114 | 1.00 | 71.46 | A | O |
| ATOM | 718 | N | PRO | A | 103 | 71.315 | 60.624 | 85.825 | 1.00 | 71.02 | A | N |
| ATOM | 719 | CD | PRO | A | 103 | 70.034 | 61.252 | 86.189 | 1.00 | 69.77 | A | C |
| ATOM | 720 | CA | PRO | A | 103 | 71.538 | 60.678 | 84.379 | 1.00 | 71.40 | A | C |
| ATOM | 721 | CB | PRO | A | 103 | 70.360 | 61.496 | 83.878 | 1.00 | 70.07 | A | C |
| ATOM | 722 | CG | PRO | A | 103 | 69.285 | 61.192 | 84.891 | 1.00 | 69.74 | A | C |
| ATOM | 723 | C | PRO | A | 103 | 72.857 | 61.426 | 84.266 | 1.00 | 72.86 | A | C |
| ATOM | 724 | O | PRO | A | 103 | 73.475 | 61.715 | 85.293 | 1.00 | 74.39 | A | O |
| ATOM | 725 | N | GLU | A | 104 | 73.273 | 61.769 | 83.051 | 1.00 | 73.24 | A | N |
| ATOM | 726 | CA | GLU | A | 104 | 74.551 | 62.453 | 82.819 | 1.00 | 72.48 | A | C |
| ATOM | 727 | CB | GLU | A | 104 | 75.021 | 63.236 | 84.052 | 1.00 | 73.14 | A | C |
| ATOM | 728 | CG | GLU | A | 104 | 74.127 | 64.396 | 84.464 | 1.00 | 75.66 | A | C |
| ATOM | 729 | CD | GLU | A | 104 | 74.212 | 65.574 | 83.514 | 1.00 | 77.84 | A | C |
| ATOM | 730 | OE1 | GLU | A | 104 | 75.326 | 66.124 | 83.342 | 1.00 | 80.31 | A | O |
| ATOM | 731 | OE2 | GLU | A | 104 | 73.165 | 65.951 | 82.943 | 1.00 | 76.19 | A | O |
| ATOM | 732 | C | GLU | A | 104 | 75.553 | 61.350 | 82.516 | 1.00 | 71.99 | A | C |
| ATOM | 733 | O | GLU | A | 104 | 76.366 | 61.465 | 81.600 | 1.00 | 72.28 | A | O |
| ATOM | 734 | N | ASP | A | 105 | 75.497 | 60.279 | 83.302 | 1.00 | 71.52 | A | N |
| ATOM | 735 | CA | ASP | A | 105 | 76.374 | 59.143 | 83.079 | 1.00 | 71.39 | A | C |
| ATOM | 736 | CB | ASP | A | 105 | 76.504 | 58.300 | 84.346 | 1.00 | 70.46 | A | C |
| ATOM | 737 | CG | ASP | A | 105 | 77.475 | 57.161 | 84.178 | 1.00 | 69.86 | A | C |
| ATOM | 738 | OD1 | ASP | A | 105 | 78.616 | 57.410 | 83.740 | 1.00 | 70.70 | A | O |
| ATOM | 739 | OD2 | ASP | A | 105 | 77.101 | 56.015 | 84.485 | 1.00 | 72.05 | A | O |
| ATOM | 740 | C | ASP | A | 105 | 75.752 | 58.320 | 81.949 | 1.00 | 71.53 | A | C |
| ATOM | 741 | O | ASP | A | 105 | 74.702 | 57.697 | 82.117 | 1.00 | 71.00 | A | O |
| ATOM | 742 | N | HIS | A | 106 | 76.407 | 58.341 | 80.795 | 1.00 | 72.05 | A | N |
| ATOM | 743 | CA | HIS | A | 106 | 75.932 | 57.632 | 79.619 | 1.00 | 72.53 | A | C |
| ATOM | 744 | CB | HIS | A | 106 | 76.730 | 58.084 | 78.400 | 1.00 | 76.15 | A | C |
| ATOM | 745 | CG | HIS | A | 106 | 76.763 | 59.574 | 78.235 | 1.00 | 79.67 | A | C |
| ATOM | 746 | CD2 | HIS | A | 106 | 77.797 | 60.450 | 78.232 | 1.00 | 80.13 | A | C |
| ATOM | 747 | ND1 | HIS | A | 106 | 75.621 | 60.329 | 78.073 | 1.00 | 80.69 | A | N |
| ATOM | 748 | CE1 | HIS | A | 106 | 75.949 | 61.605 | 77.977 | 1.00 | 81.11 | A | C |
| ATOM | 749 | NE2 | HIS | A | 106 | 77.263 | 61.706 | 78.070 | 1.00 | 81.29 | A | N |
| ATOM | 750 | C | HIS | A | 106 | 76.020 | 56.127 | 79.790 | 1.00 | 71.33 | A | C |
| ATOM | 751 | O | HIS | A | 106 | 75.883 | 55.377 | 78.824 | 1.00 | 72.43 | A | O |
| ATOM | 752 | N | ARG | A | 107 | 76.269 | 55.689 | 81.020 | 1.00 | 68.67 | A | N |
| ATOM | 753 | CA | ARG | A | 107 | 76.330 | 54.267 | 81.319 | 1.00 | 66.62 | A | C |
| ATOM | 754 | CB | ARG | A | 107 | 77.570 | 53.935 | 82.147 | 1.00 | 69.32 | A | C |
| ATOM | 755 | CG | ARG | A | 107 | 78.546 | 53.027 | 81.419 | 1.00 | 74.08 | A | C |
| ATOM | 756 | CD | ARG | A | 107 | 79.702 | 52.646 | 82.316 | 1.00 | 79.04 | A | C |
| ATOM | 757 | NE | ARG | A | 107 | 80.973 | 53.186 | 81.843 | 1.00 | 83.20 | A | N |
| ATOM | 758 | CZ | ARG | A | 107 | 81.780 | 53.952 | 82.573 | 1.00 | 85.54 | A | C |
| ATOM | 759 | NH1 | ARG | A | 107 | 81.448 | 54.277 | 83.817 | 1.00 | 85.60 | A | N |
| ATOM | 760 | NH2 | ARG | A | 107 | 82.923 | 54.388 | 82.059 | 1.00 | 86.87 | A | N |
| ATOM | 761 | C | ARG | A | 107 | 75.058 | 53.922 | 82.087 | 1.00 | 63.41 | A | C |
| ATOM | 762 | O | ARG | A | 107 | 74.503 | 52.835 | 81.932 | 1.00 | 61.99 | A | O |
| ATOM | 763 | N | THR | A | 108 | 74.595 | 54.867 | 82.903 | 1.00 | 60.02 | A | N |
| ATOM | 764 | CA | THR | A | 108 | 73.369 | 54.689 | 83.673 | 1.00 | 57.14 | A | C |
| ATOM | 765 | CB | THR | A | 108 | 73.040 | 55.939 | 84.526 | 1.00 | 56.60 | A | C |
| ATOM | 766 | OG1 | THR | A | 108 | 74.045 | 56.118 | 85.535 | 1.00 | 58.17 | A | O |
| ATOM | 767 | CG2 | THR | A | 108 | 71.680 | 55.791 | 85.190 | 1.00 | 54.01 | A | C |
| ATOM | 768 | C | THR | A | 108 | 72.242 | 54.476 | 82.665 | 1.00 | 56.35 | A | C |
| ATOM | 769 | O | THR | A | 108 | 71.508 | 53.494 | 82.736 | 1.00 | 55.93 | A | O |
| ATOM | 770 | N | LEU | A | 109 | 72.132 | 55.400 | 81.716 | 1.00 | 54.61 | A | N |
| ATOM | 771 | CA | LEU | A | 109 | 71.109 | 55.340 | 80.678 | 1.00 | 54.24 | A | C |
| ATOM | 772 | CB | LEU | A | 109 | 71.232 | 56.535 | 79.735 | 1.00 | 52.57 | A | C |
| ATOM | 773 | CG | LEU | A | 109 | 71.112 | 57.926 | 80.344 | 1.00 | 52.50 | A | C |
| ATOM | 774 | CD1 | LEU | A | 109 | 71.135 | 58.963 | 79.238 | 1.00 | 50.95 | A | C |
| ATOM | 775 | CD2 | LEU | A | 109 | 69.830 | 58.024 | 81.141 | 1.00 | 53.13 | A | C |
| ATOM | 776 | C | LEU | A | 109 | 71.204 | 54.065 | 79.856 | 1.00 | 54.20 | A | C |
| ATOM | 777 | O | LEU | A | 109 | 70.194 | 53.430 | 79.559 | 1.00 | 54.17 | A | O |
| ATOM | 778 | N | SER | A | 110 | 72.422 | 53.701 | 79.479 | 1.00 | 54.22 | A | N |
| ATOM | 779 | CA | SER | A | 110 | 72.647 | 52.502 | 78.685 | 1.00 | 53.55 | A | C |
| ATOM | 780 | CB | SER | A | 110 | 74.127 | 52.376 | 78.343 | 1.00 | 54.06 | A | C |
| ATOM | 781 | OG | SER | A | 110 | 74.368 | 51.166 | 77.652 | 1.00 | 57.11 | A | O |
| ATOM | 782 | C | SER | A | 110 | 72.175 | 51.228 | 79.386 | 1.00 | 52.97 | A | C |
| ATOM | 783 | O | SER | A | 110 | 71.517 | 50.391 | 78.774 | 1.00 | 53.06 | A | O |
| ATOM | 784 | N | GLN | A | 111 | 72.512 | 51.068 | 80.663 | 1.00 | 52.97 | A | N |
| ATOM | 785 | CA | GLN | A | 111 | 72.092 | 49.875 | 81.387 | 1.00 | 52.83 | A | C |
| ATOM | 786 | CB | GLN | A | 111 | 72.905 | 49.702 | 82.678 | 1.00 | 54.59 | A | C |
| ATOM | 787 | CG | GLN | A | 111 | 73.239 | 50.988 | 83.393 | 1.00 | 59.90 | A | C |
| ATOM | 788 | CD | GLN | A | 111 | 74.325 | 50.809 | 84.446 | 1.00 | 61.15 | A | C |
| ATOM | 789 | OE1 | GLN | A | 111 | 74.055 | 50.409 | 85.582 | 1.00 | 60.60 | A | O |
| ATOM | 790 | NE2 | GLN | A | 111 | 75.563 | 51.097 | 84.064 | 1.00 | 62.46 | A | N |
| ATOM | 791 | C | GLN | A | 111 | 70.597 | 49.893 | 81.683 | 1.00 | 50.65 | A | C |
| ATOM | 792 | O | GLN | A | 111 | 70.009 | 48.864 | 82.004 | 1.00 | 51.69 | A | O |
| ATOM | 793 | N | PHE | A | 112 | 69.981 | 51.064 | 81.561 | 1.00 | 48.38 | A | N |
| ATOM | 794 | CA | PHE | A | 112 | 68.547 | 51.197 | 81.788 | 1.00 | 45.97 | A | C |
| ATOM | 795 | CB | PHE | A | 112 | 68.166 | 52.669 | 81.933 | 1.00 | 42.79 | A | C |
| ATOM | 796 | CG | PHE | A | 112 | 66.691 | 52.904 | 82.109 | 1.00 | 39.44 | A | C |
| ATOM | 797 | CD1 | PHE | A | 112 | 66.042 | 52.501 | 83.267 | 1.00 | 37.72 | A | C |
| ATOM | 798 | CD2 | PHE | A | 112 | 65.947 | 53.531 | 81.110 | 1.00 | 38.04 | A | C |
| ATOM | 799 | CE1 | PHE | A | 112 | 64.673 | 52.714 | 83.437 | 1.00 | 36.59 | A | C |
| ATOM | 800 | CE2 | PHE | A | 112 | 64.576 | 53.750 | 81.268 | 1.00 | 37.51 | A | C |
| ATOM | 801 | CZ | PHE | A | 112 | 63.939 | 53.337 | 82.439 | 1.00 | 35.21 | A | C |
| ATOM | 802 | C | PHE | A | 112 | 67.851 | 50.594 | 80.570 | 1.00 | 46.42 | A | C |
| ATOM | 803 | O | PHE | A | 112 | 66.879 | 49.850 | 80.701 | 1.00 | 46.61 | A | O |
| ATOM | 804 | N | THR | A | 113 | 68.360 | 50.915 | 79.383 | 1.00 | 45.33 | A | N |
| ATOM | 805 | CA | THR | A | 113 | 67.797 | 50.378 | 78.154 | 1.00 | 45.88 | A | C |
| ATOM | 806 | CB | THR | A | 113 | 68.535 | 50.916 | 76.924 | 1.00 | 45.06 | A | C |
| ATOM | 807 | OG1 | THR | A | 113 | 68.316 | 52.324 | 76.829 | 1.00 | 47.44 | A | O |
| ATOM | 808 | CG2 | THR | A | 113 | 68.032 | 50.248 | 75.659 | 1.00 | 43.95 | A | C |
| ATOM | 809 | C | THR | A | 113 | 67.923 | 48.860 | 78.195 | 1.00 | 46.42 | A | C |
| ATOM | 810 | O | THR | A | 113 | 66.980 | 48.145 | 77.867 | 1.00 | 46.85 | A | O |
| ATOM | 811 | N | ASP | A | 114 | 69.093 | 48.375 | 78.598 | 1.00 | 46.16 | A | N |
| ATOM | 812 | CA | ASP | A | 114 | 69.322 | 46.945 | 78.706 | 1.00 | 46.81 | A | C |
| ATOM | 813 | CB | ASP | A | 114 | 70.717 | 46.668 | 79.279 | 1.00 | 50.60 | A | C |
| ATOM | 814 | CG | ASP | A | 114 | 71.820 | 46.836 | 78.252 | 1.00 | 54.23 | A | C |
| ATOM | 815 | OD1 | ASP | A | 114 | 73.009 | 46.731 | 78.629 | 1.00 | 58.03 | A | O |
| ATOM | 816 | OD2 | ASP | A | 114 | 71.502 | 47.061 | 77.065 | 1.00 | 56.36 | A | O |
| ATOM | 817 | C | ASP | A | 114 | 68.275 | 46.358 | 79.639 | 1.00 | 45.15 | A | C |
| ATOM | 818 | O | ASP | A | 114 | 67.675 | 45.324 | 79.348 | 1.00 | 45.24 | A | O |
| ATOM | 819 | N | ALA | A | 115 | 68.072 | 47.037 | 80.763 | 1.00 | 42.13 | A | N |
| ATOM | 820 | CA | ALA | A | 115 | 67.121 | 46.609 | 81.775 | 1.00 | 41.83 | A | C |
| ATOM | 821 | CB | ALA | A | 115 | 67.108 | 47.611 | 82.927 | 1.00 | 39.58 | A | C |
| ATOM | 822 | C | ALA | A | 115 | 65.712 | 46.446 | 81.199 | 1.00 | 42.52 | A | C |
| ATOM | 823 | O | ALA | A | 115 | 65.012 | 45.476 | 81.521 | 1.00 | 41.67 | A | O |
| ATOM | 824 | N | LEU | A | 116 | 65.303 | 47.390 | 80.349 | 1.00 | 41.07 | A | N |
| ATOM | 825 | CA | LEU | A | 116 | 63.974 | 47.349 | 79.739 | 1.00 | 40.35 | A | C |
| ATOM | 826 | CB | LEU | A | 116 | 63.673 | 48.652 | 78.997 | 1.00 | 38.31 | A | C |
| ATOM | 827 | CG | LEU | A | 116 | 63.620 | 49.894 | 79.890 | 1.00 | 35.53 | A | C |
| ATOM | 828 | CD1 | LEU | A | 116 | 63.291 | 51.088 | 79.041 | 1.00 | 37.29 | A | C |
| ATOM | 829 | CD2 | LEU | A | 116 | 62.592 | 49.736 | 80.997 | 1.00 | 35.86 | A | C |
| ATOM | 830 | C | LEU | A | 116 | 63.785 | 46.176 | 78.789 | 1.00 | 41.27 | A | C |
| ATOM | 831 | O | LEU | A | 116 | 62.772 | 45.483 | 78.863 | 1.00 | 43.00 | A | O |
| ATOM | 832 | N | VAL | A | 117 | 64.750 | 45.954 | 77.900 | 1.00 | 39.81 | A | N |
| ATOM | 833 | CA | VAL | A | 117 | 64.670 | 44.846 | 76.954 | 1.00 | 39.36 | A | C |
| ATOM | 834 | CB | VAL | A | 117 | 65.910 | 44.783 | 76.034 | 1.00 | 39.66 | A | C |
| ATOM | 835 | CG1 | VAL | A | 117 | 65.929 | 43.462 | 75.272 | 1.00 | 38.47 | A | C |
| ATOM | 836 | CG2 | VAL | A | 117 | 65.883 | 45.944 | 75.051 | 1.00 | 38.17 | A | C |
| ATOM | 837 | C | VAL | A | 117 | 64.546 | 43.519 | 77.689 | 1.00 | 39.79 | A | C |
| ATOM | 838 | O | VAL | A | 117 | 63.754 | 42.660 | 77.303 | 1.00 | 40.05 | A | O |
| ATOM | 839 | N | THR | A | 118 | 65.335 | 43.354 | 78.744 | 1.00 | 39.13 | A | N |
| ATOM | 840 | CA | THR | A | 118 | 65.299 | 42.131 | 79.531 | 1.00 | 40.21 | A | C |
| ATOM | 841 | CB | THR | A | 118 | 66.426 | 42.130 | 80.586 | 1.00 | 39.85 | A | C |
| ATOM | 842 | OG1 | THR | A | 118 | 67.665 | 41.799 | 79.942 | 1.00 | 43.25 | A | O |
| ATOM | 843 | CG2 | THR | A | 118 | 66.132 | 41.132 | 81.706 | 1.00 | 39.33 | A | C |
| ATOM | 844 | C | THR | A | 118 | 63.929 | 41.968 | 80.192 | 1.00 | 39.25 | A | C |
| ATOM | 845 | O | THR | A | 118 | 63.401 | 40.861 | 80.274 | 1.00 | 40.93 | A | O |
| ATOM | 846 | N | ILE | A | 119 | 63.348 | 43.072 | 80.647 | 1.00 | 38.36 | A | N |
| ATOM | 847 | CA | ILE | A | 119 | 62.031 | 43.012 | 81.265 | 1.00 | 37.36 | A | C |
| ATOM | 848 | CB | ILE | A | 119 | 61.627 | 44.384 | 81.848 | 1.00 | 34.50 | A | C |
| ATOM | 849 | CG2 | ILE | A | 119 | 60.134 | 44.424 | 82.132 | 1.00 | 32.54 | A | C |
| ATOM | 850 | CG1 | ILE | A | 119 | 62.412 | 44.647 | 83.135 | 1.00 | 33.95 | A | C |
| ATOM | 851 | CD1 | ILE | A | 119 | 62.157 | 46.016 | 83.752 | 1.00 | 32.60 | A | C |
| ATOM | 852 | C | ILE | A | 119 | 61.025 | 42.583 | 80.195 | 1.00 | 38.26 | A | C |
| ATOM | 853 | O | ILE | A | 119 | 60.192 | 41.704 | 80.423 | 1.00 | 36.95 | A | O |
| ATOM | 854 | N | ARG | A | 120 | 61.134 | 43.189 | 79.018 | 1.00 | 38.70 | A | N |
| ATOM | 855 | CA | ARG | A | 120 | 60.242 | 42.890 | 77.907 | 1.00 | 40.30 | A | C |
| ATOM | 856 | CB | ARG | A | 120 | 60.672 | 43.667 | 76.666 | 1.00 | 42.02 | A | C |
| ATOM | 857 | CG | ARG | A | 120 | 59.541 | 44.212 | 75.814 | 1.00 | 42.27 | A | C |
| ATOM | 858 | CD | ARG | A | 120 | 59.711 | 43.788 | 74.356 | 1.00 | 46.00 | A | C |
| ATOM | 859 | NE | ARG | A | 120 | 61.084 | 43.939 | 73.865 | 1.00 | 48.58 | A | N |
| ATOM | 860 | CZ | ARG | A | 120 | 61.566 | 45.012 | 73.239 | 1.00 | 50.18 | A | C |
| ATOM | 861 | NH1 | ARG | A | 120 | 60.793 | 46.064 | 73.007 | 1.00 | 51.06 | A | N |
| ATOM | 862 | NH2 | ARG | A | 120 | 62.833 | 45.034 | 72.841 | 1.00 | 51.06 | A | N |
| ATOM | 863 | C | ARG | A | 120 | 60.236 | 41.404 | 77.585 | 1.00 | 40.75 | A | C |
| ATOM | 864 | O | ARG | A | 120 | 59.174 | 40.811 | 77.412 | 1.00 | 39.69 | A | O |
| ATOM | 865 | N | ASN | A | 121 | 61.421 | 40.806 | 77.495 | 1.00 | 41.05 | A | N |
| ATOM | 866 | CA | ASN | A | 121 | 61.530 | 39.383 | 77.190 | 1.00 | 41.64 | A | C |
| ATOM | 867 | CB | ASN | A | 121 | 62.995 | 38.967 | 77.045 | 1.00 | 45.50 | A | C |
| ATOM | 868 | CG | ASN | A | 121 | 63.732 | 39.798 | 76.024 | 1.00 | 50.34 | A | C |
| ATOM | 869 | OD1 | ASN | A | 121 | 63.173 | 40.151 | 74.981 | 1.00 | 49.61 | A | O |
| ATOM | 870 | ND2 | ASN | A | 121 | 64.997 | 40.108 | 76.307 | 1.00 | 51.66 | A | N |
| ATOM | 871 | C | ASN | A | 121 | 60.906 | 38.554 | 78.294 | 1.00 | 39.91 | A | C |
| ATOM | 872 | O | ASN | A | 121 | 60.172 | 37.600 | 78.043 | 1.00 | 41.34 | A | O |
| ATOM | 873 | N | ARG | A | 122 | 61.219 | 38.927 | 79.523 | 1.00 | 37.58 | A | N |
| ATOM | 874 | CA | ARG | A | 122 | 60.712 | 38.221 | 80.678 | 1.00 | 37.99 | A | C |
| ATOM | 875 | CB | ARG | A | 122 | 61.124 | 38.982 | 81.947 | 1.00 | 37.87 | A | C |
| ATOM | 876 | CG | ARG | A | 122 | 60.690 | 38.343 | 83.255 | 1.00 | 39.31 | A | C |
| ATOM | 877 | CD | ARG | A | 122 | 61.744 | 38.544 | 84.338 | 1.00 | 41.89 | A | C |
| ATOM | 878 | NE | ARG | A | 122 | 62.005 | 39.944 | 84.666 | 1.00 | 44.59 | A | N |
| ATOM | 879 | CZ | ARG | A | 122 | 63.225 | 40.446 | 84.824 | 1.00 | 45.41 | A | C |
| ATOM | 880 | NH1 | ARG | A | 122 | 64.291 | 39.667 | 84.673 | 1.00 | 46.46 | A | N |
| ATOM | 881 | NH2 | ARG | A | 122 | 63.383 | 41.720 | 85.154 | 1.00 | 47.40 | A | N |
| ATOM | 882 | C | ARG | A | 122 | 59.190 | 38.041 | 80.603 | 1.00 | 37.08 | A | C |
| ATOM | 883 | O | ARG | A | 122 | 58.667 | 36.977 | 80.944 | 1.00 | 38.06 | A | O |
| ATOM | 884 | N | HIS | A | 123 | 58.495 | 39.065 | 80.111 | 1.00 | 35.08 | A | N |
| ATOM | 885 | CA | HIS | A | 123 | 57.035 | 39.067 | 80.010 | 1.00 | 31.94 | A | C |
| ATOM | 886 | CB | HIS | A | 123 | 56.527 | 40.473 | 80.311 | 1.00 | 29.01 | A | C |
| ATOM | 887 | CG | HIS | A | 123 | 56.826 | 40.935 | 81.699 | 1.00 | 26.92 | A | C |
| ATOM | 888 | CD2 | HIS | A | 123 | 57.987 | 41.313 | 82.283 | 1.00 | 25.70 | A | C |
| ATOM | 889 | ND1 | HIS | A | 123 | 55.864 | 40.998 | 82.682 | 1.00 | 27.52 | A | N |
| ATOM | 890 | CE1 | HIS | A | 123 | 56.420 | 41.394 | 83.814 | 1.00 | 28.00 | A | C |
| ATOM | 891 | NE2 | HIS | A | 123 | 57.708 | 41.590 | 83.599 | 1.00 | 26.22 | A | N |
| ATOM | 892 | C | HIS | A | 123 | 56.458 | 38.617 | 78.669 | 1.00 | 32.57 | A | C |
| ATOM | 893 | O | HIS | A | 123 | 55.239 | 38.632 | 78.470 | 1.00 | 31.43 | A | O |
| ATOM | 894 | N | ASN | A | 124 | 57.322 | 38.218 | 77.752 | 1.00 | 32.75 | A | N |
| ATOM | 895 | CA | ASN | A | 124 | 56.863 | 37.814 | 76.436 | 1.00 | 36.64 | A | C |
| ATOM | 896 | CB | ASN | A | 124 | 58.043 | 37.314 | 75.607 | 1.00 | 41.45 | A | C |
| ATOM | 897 | CG | ASN | A | 124 | 58.377 | 38.245 | 74.460 | 1.00 | 47.09 | A | C |
| ATOM | 898 | OD1 | ASN | A | 124 | 57.543 | 38.491 | 73.584 | 1.00 | 52.68 | A | O |
| ATOM | 899 | ND2 | ASN | A | 124 | 59.599 | 38.770 | 74.455 | 1.00 | 49.16 | A | N |
| ATOM | 900 | C | ASN | A | 124 | 55.744 | 36.785 | 76.392 | 1.00 | 36.05 | A | C |
| ATOM | 901 | O | ASN | A | 124 | 54.937 | 36.806 | 75.473 | 1.00 | 38.31 | A | O |
| ATOM | 902 | N | ASP | A | 125 | 55.672 | 35.892 | 77.371 | 1.00 | 35.87 | A | N |
| ATOM | 903 | CA | ASP | A | 125 | 54.632 | 34.872 | 77.345 | 1.00 | 35.39 | A | C |
| ATOM | 904 | CB | ASP | A | 125 | 55.245 | 33.480 | 77.547 | 1.00 | 34.83 | A | C |
| ATOM | 905 | CG | ASP | A | 125 | 56.279 | 33.138 | 76.495 | 1.00 | 36.72 | A | C |
| ATOM | 906 | OD1 | ASP | A | 125 | 56.028 | 33.405 | 75.299 | 1.00 | 35.47 | A | O |
| ATOM | 907 | OD2 | ASP | A | 125 | 57.339 | 32.594 | 76.865 | 1.00 | 39.60 | A | O |
| ATOM | 908 | C | ASP | A | 125 | 53.524 | 35.073 | 78.369 | 1.00 | 36.17 | A | C |
| ATOM | 909 | O | ASP | A | 125 | 52.904 | 34.097 | 78.807 | 1.00 | 36.40 | A | O |
| ATOM | 910 | N | VAL | A | 126 | 53.263 | 36.323 | 78.748 | 1.00 | 33.65 | A | N |
| ATOM | 911 | CA | VAL | A | 126 | 52.220 | 36.596 | 79.732 | 1.00 | 32.17 | A | C |
| ATOM | 912 | CB | VAL | A | 126 | 52.168 | 38.097 | 80.108 | 1.00 | 32.08 | A | C |
| ATOM | 913 | CG1 | VAL | A | 126 | 50.891 | 38.400 | 80.866 | 1.00 | 31.38 | A | C |
| ATOM | 914 | CG2 | VAL | A | 126 | 53.353 | 38.458 | 80.980 | 1.00 | 29.56 | A | C |
| ATOM | 915 | C | VAL | A | 126 | 50.829 | 36.155 | 79.275 | 1.00 | 31.20 | A | C |
| ATOM | 916 | O | VAL | A | 126 | 50.085 | 35.531 | 80.041 | 1.00 | 30.63 | A | O |
| ATOM | 917 | N | VAL | A | 127 | 50.476 | 36.460 | 78.031 | 1.00 | 29.83 | A | N |
| ATOM | 918 | CA | VAL | A | 127 | 49.153 | 36.097 | 77.537 | 1.00 | 28.68 | A | C |
| ATOM | 919 | CB | VAL | A | 127 | 48.919 | 36.616 | 76.078 | 1.00 | 28.68 | A | C |
| ATOM | 920 | CG1 | VAL | A | 127 | 47.539 | 36.192 | 75.578 | 1.00 | 24.57 | A | C |
| ATOM | 921 | CG2 | VAL | A | 127 | 49.044 | 38.149 | 76.047 | 1.00 | 27.37 | A | C |
| ATOM | 922 | C | VAL | A | 127 | 48.885 | 34.596 | 77.631 | 1.00 | 28.41 | A | C |
| ATOM | 923 | O | VAL | A | 127 | 48.004 | 34.181 | 78.374 | 1.00 | 30.16 | A | O |
| ATOM | 924 | N | PRO | A | 128 | 49.646 | 33.758 | 76.905 | 1.00 | 29.32 | A | N |
| ATOM | 925 | CD | PRO | A | 128 | 50.806 | 33.990 | 76.029 | 1.00 | 28.63 | A | C |
| ATOM | 926 | CA | PRO | A | 128 | 49.365 | 32.323 | 77.014 | 1.00 | 27.07 | A | C |
| ATOM | 927 | CB | PRO | A | 128 | 50.423 | 31.690 | 76.108 | 1.00 | 25.87 | A | C |
| ATOM | 928 | CG | PRO | A | 128 | 51.530 | 32.663 | 76.131 | 1.00 | 29.29 | A | C |
| ATOM | 929 | C | PRO | A | 128 | 49.403 | 31.786 | 78.450 | 1.00 | 29.36 | A | C |
| ATOM | 930 | O | PRO | A | 128 | 48.581 | 30.937 | 78.828 | 1.00 | 29.18 | A | O |
| ATOM | 931 | N | THR | A | 129 | 50.341 | 32.293 | 79.248 | 1.00 | 28.44 | A | N |
| ATOM | 932 | CA | THR | A | 129 | 50.478 | 31.855 | 80.635 | 1.00 | 28.53 | A | C |
| ATOM | 933 | CB | THR | A | 129 | 51.657 | 32.548 | 81.340 | 1.00 | 29.34 | A | C |
| ATOM | 934 | OG1 | THR | A | 129 | 52.879 | 32.207 | 80.677 | 1.00 | 33.06 | A | O |
| ATOM | 935 | CG2 | THR | A | 129 | 51.742 | 32.102 | 82.782 | 1.00 | 25.75 | A | C |
| ATOM | 936 | C | THR | A | 129 | 49.203 | 32.148 | 81.411 | 1.00 | 30.19 | A | C |
| ATOM | 937 | O | THR | A | 129 | 48.700 | 31.293 | 82.129 | 1.00 | 29.67 | A | O |
| ATOM | 938 | N | MET | A | 130 | 48.673 | 33.355 | 81.269 | 1.00 | 30.30 | A | N |
| ATOM | 939 | CA | MET | A | 130 | 47.433 | 33.694 | 81.956 | 1.00 | 29.65 | A | C |
| ATOM | 940 | CB | MET | A | 130 | 47.166 | 35.196 | 81.827 | 1.00 | 29.48 | A | C |
| ATOM | 941 | CG | MET | A | 130 | 48.023 | 36.036 | 82.748 | 1.00 | 31.60 | A | C |
| ATOM | 942 | SD | MET | A | 130 | 47.524 | 35.782 | 84.434 | 1.00 | 35.79 | A | S |
| ATOM | 943 | CE | MET | A | 130 | 48.345 | 37.078 | 85.222 | 1.00 | 36.72 | A | C |
| ATOM | 944 | C | MET | A | 130 | 46.253 | 32.883 | 81.385 | 1.00 | 29.57 | A | C |
| ATOM | 945 | O | MET | A | 130 | 45.318 | 32.529 | 82.112 | 1.00 | 28.94 | A | O |
| ATOM | 946 | N | ALA | A | 131 | 46.296 | 32.595 | 80.085 | 1.00 | 28.45 | A | N |
| ATOM | 947 | CA | ALA | A | 131 | 45.233 | 31.819 | 79.447 | 1.00 | 29.21 | A | C |
| ATOM | 948 | CB | ALA | A | 131 | 45.484 | 31.686 | 77.953 | 1.00 | 25.09 | A | C |
| ATOM | 949 | C | ALA | A | 131 | 45.178 | 30.432 | 80.089 | 1.00 | 31.27 | A | C |
| ATOM | 950 | O | ALA | A | 131 | 44.104 | 29.843 | 80.229 | 1.00 | 31.05 | A | O |
| ATOM | 951 | N | GLN | A | 132 | 46.345 | 29.914 | 80.467 | 1.00 | 31.41 | A | N |
| ATOM | 952 | CA | GLN | A | 132 | 46.447 | 28.612 | 81.115 | 1.00 | 32.74 | A | C |
| ATOM | 953 | CB | GLN | A | 132 | 47.925 | 28.254 | 81.300 | 1.00 | 32.18 | A | C |
| ATOM | 954 | CG | GLN | A | 132 | 48.227 | 26.941 | 82.015 | 1.00 | 35.03 | A | C |
| ATOM | 955 | CD | GLN | A | 132 | 47.569 | 25.715 | 81.382 | 1.00 | 39.35 | A | C |
| ATOM | 956 | OE1 | GLN | A | 132 | 47.472 | 25.592 | 80.151 | 1.00 | 40.36 | A | O |
| ATOM | 957 | NE2 | GLN | A | 132 | 47.128 | 24.788 | 82.232 | 1.00 | 38.82 | A | N |
| ATOM | 958 | C | GLN | A | 132 | 45.727 | 28.676 | 82.467 | 1.00 | 33.34 | A | C |
| ATOM | 959 | O | GLN | A | 132 | 45.162 | 27.690 | 82.938 | 1.00 | 34.94 | A | O |
| ATOM | 960 | N | GLY | A | 133 | 45.738 | 29.850 | 83.088 | 1.00 | 33.66 | A | N |
| ATOM | 961 | CA | GLY | A | 133 | 45.062 | 30.002 | 84.363 | 1.00 | 32.00 | A | C |
| ATOM | 962 | C | GLY | A | 133 | 43.555 | 29.949 | 84.195 | 1.00 | 33.38 | A | C |
| ATOM | 963 | O | GLY | A | 133 | 42.835 | 29.459 | 85.071 | 1.00 | 34.53 | A | O |
| ATOM | 964 | N | VAL | A | 134 | 43.069 | 30.457 | 83.067 | 1.00 | 33.70 | A | N |
| ATOM | 965 | CA | VAL | A | 134 | 41.634 | 30.450 | 82.794 | 1.00 | 33.72 | A | C |
| ATOM | 966 | CB | VAL | A | 134 | 41.287 | 31.294 | 81.550 | 1.00 | 31.28 | A | C |
| ATOM | 967 | CG1 | VAL | A | 134 | 39.792 | 31.225 | 81.274 | 1.00 | 32.74 | A | C |
| ATOM | 968 | CG2 | VAL | A | 134 | 41.714 | 32.730 | 81.760 | 1.00 | 30.55 | A | C |
| ATOM | 969 | C | VAL | A | 134 | 41.149 | 29.020 | 82.561 | 1.00 | 33.60 | A | C |
| ATOM | 970 | O | VAL | A | 134 | 40.113 | 28.619 | 83.087 | 1.00 | 33.82 | A | O |
| ATOM | 971 | N | LEU | A | 135 | 41.896 | 28.258 | 81.768 | 1.00 | 32.91 | A | N |
| ATOM | 972 | CA | LEU | A | 135 | 41.525 | 26.878 | 81.491 | 1.00 | 35.38 | A | C |
| ATOM | 973 | CB | LEU | A | 135 | 42.523 | 26.227 | 80.525 | 1.00 | 35.51 | A | C |
| ATOM | 974 | CG | LEU | A | 135 | 42.109 | 24.831 | 80.047 | 1.00 | 38.23 | A | C |
| ATOM | 975 | CD1 | LEU | A | 135 | 40.702 | 24.857 | 79.447 | 1.00 | 38.61 | A | C |
| ATOM | 976 | CD2 | LEU | A | 135 | 43.103 | 24.347 | 79.018 | 1.00 | 39.01 | A | C |
| ATOM | 977 | C | LEU | A | 135 | 41.501 | 26.111 | 82.809 | 1.00 | 35.54 | A | C |
| ATOM | 978 | O | LEU | A | 135 | 40.581 | 25.341 | 83.087 | 1.00 | 37.08 | A | O |
| ATOM | 979 | N | GLU | A | 136 | 42.515 | 26.335 | 83.630 | 1.00 | 34.21 | A | N |
| ATOM | 980 | CA | GLU | A | 136 | 42.578 | 25.669 | 84.916 | 1.00 | 33.93 | A | C |
| ATOM | 981 | CB | GLU | A | 136 | 43.829 | 26.086 | 85.668 | 1.00 | 34.79 | A | C |
| ATOM | 982 | CG | GLU | A | 136 | 44.826 | 24.972 | 85.780 | 1.00 | 37.19 | A | C |
| ATOM | 983 | CD | GLU | A | 136 | 46.236 | 25.484 | 85.916 | 1.00 | 37.37 | A | C |
| ATOM | 984 | OE1 | GLU | A | 136 | 46.521 | 26.197 | 86.903 | 1.00 | 35.52 | A | O |
| ATOM | 985 | OE2 | GLU | A | 136 | 47.061 | 25.173 | 85.027 | 1.00 | 38.57 | A | O |
| ATOM | 986 | C | GLU | A | 136 | 41.362 | 25.997 | 85.759 | 1.00 | 34.80 | A | C |
| ATOM | 987 | O | GLU | A | 136 | 40.752 | 25.103 | 86.344 | 1.00 | 34.09 | A | O |
| ATOM | 988 | N | TYR | A | 137 | 41.011 | 27.279 | 85.823 | 1.00 | 33.87 | A | N |
| ATOM | 989 | CA | TYR | A | 137 | 39.864 | 27.704 | 86.607 | 1.00 | 33.21 | A | C |
| ATOM | 990 | CB | TYR | A | 137 | 39.786 | 29.233 | 86.628 | 1.00 | 32.85 | A | C |
| ATOM | 991 | CG | TYR | A | 137 | 38.643 | 29.766 | 87.458 | 1.00 | 31.64 | A | C |
| ATOM | 992 | CD1 | TYR | A | 137 | 37.340 | 29.809 | 86.950 | 1.00 | 31.73 | A | C |
| ATOM | 993 | CE1 | TYR | A | 137 | 36.280 | 30.253 | 87.729 | 1.00 | 31.08 | A | C |
| ATOM | 994 | CD2 | TYR | A | 137 | 38.853 | 30.186 | 88.766 | 1.00 | 29.88 | A | C |
| ATOM | 995 | CE2 | TYR | A | 137 | 37.804 | 30.631 | 89.550 | 1.00 | 31.33 | A | C |
| ATOM | 996 | CZ | TYR | A | 137 | 36.519 | 30.660 | 89.030 | 1.00 | 32.53 | A | C |
| ATOM | 997 | OH | TYR | A | 137 | 35.472 | 31.076 | 89.821 | 1.00 | 34.79 | A | O |
| ATOM | 998 | C | TYR | A | 137 | 38.547 | 27.100 | 86.091 | 1.00 | 33.83 | A | C |
| ATOM | 999 | O | TYR | A | 137 | 37.691 | 26.708 | 86.884 | 1.00 | 33.04 | A | O |
| ATOM | 1000 | N | LYS | A | 138 | 38.384 | 27.017 | 84.773 | 1.00 | 33.48 | A | N |
| ATOM | 1001 | CA | LYS | A | 138 | 37.162 | 26.449 | 84.195 | 1.00 | 36.04 | A | C |
| ATOM | 1002 | CB | LYS | A | 138 | 37.213 | 26.485 | 82.660 | 1.00 | 34.80 | A | C |
| ATOM | 1003 | CG | LYS | A | 138 | 37.145 | 27.881 | 82.040 | 1.00 | 38.98 | A | C |
| ATOM | 1004 | CD | LYS | A | 138 | 37.176 | 27.842 | 80.504 | 1.00 | 40.66 | A | C |
| ATOM | 1005 | CE | LYS | A | 138 | 35.961 | 27.124 | 79.918 | 1.00 | 41.27 | A | C |
| ATOM | 1006 | NZ | LYS | A | 138 | 36.134 | 26.846 | 78.463 | 1.00 | 44.58 | A | N |
| ATOM | 1007 | C | LYS | A | 138 | 36.919 | 25.005 | 84.646 | 1.00 | 37.33 | A | C |
| ATOM | 1008 | O | LYS | A | 138 | 35.787 | 24.622 | 84.941 | 1.00 | 37.74 | A | O |
| ATOM | 1009 | N | ASP | A | 139 | 37.977 | 24.203 | 84.690 | 1.00 | 38.49 | A | N |
| ATOM | 1010 | CA | ASP | A | 139 | 37.838 | 22.811 | 85.103 | 1.00 | 39.72 | A | C |
| ATOM | 1011 | CB | ASP | A | 139 | 38.932 | 21.961 | 84.469 | 1.00 | 39.02 | A | C |
| ATOM | 1012 | CG | ASP | A | 139 | 38.925 | 22.053 | 82.969 | 1.00 | 40.62 | A | C |
| ATOM | 1013 | OD1 | ASP | A | 139 | 37.822 | 22.069 | 82.385 | 1.00 | 43.48 | A | O |
| ATOM | 1014 | OD2 | ASP | A | 139 | 40.016 | 22.104 | 82.373 | 1.00 | 40.92 | A | O |
| ATOM | 1015 | C | ASP | A | 139 | 37.878 | 22.638 | 86.612 | 1.00 | 38.27 | A | C |
| ATOM | 1016 | O | ASP | A | 139 | 37.299 | 21.702 | 87.152 | 1.00 | 41.08 | A | O |
| ATOM | 1017 | N | THR | A | 140 | 38.557 | 23.546 | 87.293 | 1.00 | 35.85 | A | N |
| ATOM | 1018 | CA | THR | A | 140 | 38.672 | 23.463 | 88.735 | 1.00 | 36.31 | A | C |
| ATOM | 1019 | CB | THR | A | 140 | 39.936 | 24.200 | 89.214 | 1.00 | 37.42 | A | C |
| ATOM | 1020 | OG1 | THR | A | 140 | 41.094 | 23.598 | 88.619 | 1.00 | 41.70 | A | O |
| ATOM | 1021 | CG2 | THR | A | 140 | 40.058 | 24.110 | 90.727 | 1.00 | 44.41 | A | C |
| ATOM | 1022 | C | THR | A | 140 | 37.450 | 24.027 | 89.477 | 1.00 | 35.65 | A | C |
| ATOM | 1023 | O | THR | A | 140 | 37.138 | 23.595 | 90.597 | 1.00 | 32.98 | A | O |
| ATOM | 1024 | N | TYR | A | 141 | 36.755 | 24.981 | 88.855 | 1.00 | 35.27 | A | N |
| ATOM | 1025 | CA | TYR | A | 141 | 35.598 | 25.601 | 89.500 | 1.00 | 35.30 | A | C |
| ATOM | 1026 | CB | TYR | A | 141 | 35.996 | 26.936 | 90.150 | 1.00 | 33.88 | A | C |
| ATOM | 1027 | CG | TYR | A | 141 | 37.012 | 26.777 | 91.259 | 1.00 | 36.17 | A | C |
| ATOM | 1028 | CD1 | TYR | A | 141 | 38.355 | 27.100 | 91.049 | 1.00 | 36.31 | A | C |
| ATOM | 1029 | CE1 | TYR | A | 141 | 39.315 | 26.865 | 92.037 | 1.00 | 40.16 | A | C |
| ATOM | 1030 | CD2 | TYR | A | 141 | 36.650 | 26.220 | 92.497 | 1.00 | 35.17 | A | C |
| ATOM | 1031 | CE2 | TYR | A | 141 | 37.605 | 25.981 | 93.488 | 1.00 | 36.90 | A | C |
| ATOM | 1032 | CZ | TYR | A | 141 | 38.932 | 26.303 | 93.248 | 1.00 | 39.31 | A | C |
| ATOM | 1033 | OH | TYR | A | 141 | 39.880 | 26.051 | 94.209 | 1.00 | 44.72 | A | O |
| ATOM | 1034 | C | TYR | A | 141 | 34.368 | 25.808 | 88.628 | 1.00 | 35.56 | A | C |
| ATOM | 1035 | O | TYR | A | 141 | 33.286 | 26.081 | 89.147 | 1.00 | 34.47 | A | O |
| ATOM | 1036 | N | GLY | A | 142 | 34.520 | 25.693 | 87.312 | 1.00 | 35.70 | A | N |
| ATOM | 1037 | CA | GLY | A | 142 | 33.362 | 25.840 | 86.451 | 1.00 | 37.44 | A | C |
| ATOM | 1038 | C | GLY | A | 142 | 33.318 | 27.093 | 85.613 | 1.00 | 38.83 | A | C |
| ATOM | 1039 | O | GLY | A | 142 | 34.127 | 27.996 | 85.792 | 1.00 | 38.30 | A | O |
| ATOM | 1040 | N | ASP | A | 143 | 32.346 | 27.144 | 84.707 | 1.00 | 41.91 | A | N |
| ATOM | 1041 | CA | ASP | A | 143 | 32.176 | 28.279 | 83.806 | 1.00 | 44.96 | A | C |
| ATOM | 1042 | CB | ASP | A | 143 | 32.329 | 27.804 | 82.368 | 1.00 | 47.20 | A | C |
| ATOM | 1043 | CG | ASP | A | 143 | 31.628 | 26.488 | 82.120 | 1.00 | 51.92 | A | C |
| ATOM | 1044 | OD1 | ASP | A | 143 | 30.572 | 26.246 | 82.748 | 1.00 | 54.56 | A | O |
| ATOM | 1045 | OD2 | ASP | A | 143 | 32.131 | 25.700 | 81.290 | 1.00 | 53.73 | A | O |
| ATOM | 1046 | C | ASP | A | 143 | 30.857 | 29.040 | 83.944 | 1.00 | 45.47 | A | C |
| ATOM | 1047 | O | ASP | A | 143 | 30.166 | 29.267 | 82.955 | 1.00 | 46.02 | A | O |
| ATOM | 1048 | N | ASP | A | 144 | 30.521 | 29.444 | 85.164 | 1.00 | 45.61 | A | N |
| ATOM | 1049 | CA | ASP | A | 144 | 29.290 | 30.191 | 85.417 | 1.00 | 46.88 | A | C |
| ATOM | 1050 | CB | ASP | A | 144 | 28.989 | 30.202 | 86.913 | 1.00 | 45.77 | A | C |
| ATOM | 1051 | CG | ASP | A | 144 | 30.138 | 30.743 | 87.721 | 1.00 | 45.60 | A | C |
| ATOM | 1052 | OD1 | ASP | A | 144 | 31.171 | 30.046 | 87.814 | 1.00 | 49.70 | A | O |
| ATOM | 1053 | OD2 | ASP | A | 144 | 30.019 | 31.867 | 88.251 | 1.00 | 45.64 | A | O |
| ATOM | 1054 | C | ASP | A | 144 | 29.456 | 31.636 | 84.928 | 1.00 | 47.58 | A | C |
| ATOM | 1055 | O | ASP | A | 144 | 30.579 | 32.130 | 84.824 | 1.00 | 49.07 | A | O |
| ATOM | 1056 | N | PRO | A | 145 | 28.338 | 32.331 | 84.631 | 1.00 | 46.00 | A | N |
| ATOM | 1057 | CD | PRO | A | 145 | 26.963 | 31.798 | 84.595 | 1.00 | 45.22 | A | C |
| ATOM | 1058 | CA | PRO | A | 145 | 28.355 | 33.720 | 84.154 | 1.00 | 45.20 | A | C |
| ATOM | 1059 | CB | PRO | A | 145 | 26.871 | 34.065 | 84.054 | 1.00 | 44.87 | A | C |
| ATOM | 1060 | CG | PRO | A | 145 | 26.264 | 32.775 | 83.663 | 1.00 | 45.66 | A | C |
| ATOM | 1061 | C | PRO | A | 145 | 29.112 | 34.712 | 85.043 | 1.00 | 45.08 | A | C |
| ATOM | 1062 | O | PRO | A | 145 | 29.755 | 35.639 | 84.538 | 1.00 | 45.27 | A | O |
| ATOM | 1063 | N | VAL | A | 146 | 29.026 | 34.534 | 86.361 | 1.00 | 44.80 | A | N |
| ATOM | 1064 | CA | VAL | A | 146 | 29.713 | 35.430 | 87.286 | 1.00 | 43.56 | A | C |
| ATOM | 1065 | CB | VAL | A | 146 | 29.197 | 35.251 | 88.711 | 1.00 | 42.39 | A | C |
| ATOM | 1066 | CG1 | VAL | A | 146 | 29.975 | 36.134 | 89.668 | 1.00 | 40.99 | A | C |
| ATOM | 1067 | CG2 | VAL | A | 146 | 27.740 | 35.627 | 88.752 | 1.00 | 42.35 | A | C |
| ATOM | 1068 | C | VAL | A | 146 | 31.225 | 35.237 | 87.254 | 1.00 | 43.68 | A | C |
| ATOM | 1069 | O | VAL | A | 146 | 31.980 | 36.184 | 87.487 | 1.00 | 43.92 | A | O |
| ATOM | 1070 | N | SER | A | 147 | 31.665 | 34.013 | 86.966 | 1.00 | 42.87 | A | N |
| ATOM | 1071 | CA | SER | A | 147 | 33.098 | 33.724 | 86.859 | 1.00 | 42.67 | A | C |
| ATOM | 1072 | CB | SER | A | 147 | 33.352 | 32.215 | 86.792 | 1.00 | 41.15 | A | C |
| ATOM | 1073 | OG | SER | A | 147 | 33.121 | 31.609 | 88.051 | 1.00 | 45.97 | A | O |
| ATOM | 1074 | C | SER | A | 147 | 33.585 | 34.371 | 85.568 | 1.00 | 41.92 | A | C |
| ATOM | 1075 | O | SER | A | 147 | 34.687 | 34.914 | 85.497 | 1.00 | 42.12 | A | O |
| ATOM | 1076 | N | ASN | A | 148 | 32.744 | 34.304 | 84.546 | 1.00 | 40.24 | A | N |
| ATOM | 1077 | CA | ASN | A | 148 | 33.059 | 34.885 | 83.252 | 1.00 | 41.08 | A | C |
| ATOM | 1078 | CB | ASN | A | 148 | 31.941 | 34.579 | 82.268 | 1.00 | 39.94 | A | C |
| ATOM | 1079 | CG | ASN | A | 148 | 32.243 | 35.091 | 80.886 | 1.00 | 41.52 | A | C |
| ATOM | 1080 | OD1 | ASN | A | 148 | 33.141 | 34.583 | 80.216 | 1.00 | 42.59 | A | O |
| ATOM | 1081 | ND2 | ASN | A | 148 | 31.510 | 36.117 | 80.452 | 1.00 | 40.20 | A | N |
| ATOM | 1082 | C | ASN | A | 148 | 33.220 | 36.404 | 83.338 | 1.00 | 41.40 | A | C |
| ATOM | 1083 | O | ASN | A | 148 | 34.106 | 36.991 | 82.717 | 1.00 | 40.91 | A | O |
| ATOM | 1084 | N | GLN | A | 149 | 32.336 | 37.026 | 84.103 | 1.00 | 41.44 | A | N |
| ATOM | 1085 | CA | GLN | A | 149 | 32.329 | 38.463 | 84.288 | 1.00 | 43.45 | A | C |
| ATOM | 1086 | CB | GLN | A | 149 | 31.068 | 38.827 | 85.057 | 1.00 | 47.85 | A | C |
| ATOM | 1087 | CG | GLN | A | 149 | 31.016 | 40.198 | 85.653 | 1.00 | 54.95 | A | C |
| ATOM | 1088 | CD | GLN | A | 149 | 29.659 | 40.468 | 86.286 | 1.00 | 59.88 | A | C |
| ATOM | 1089 | OE1 | GLN | A | 149 | 29.495 | 41.429 | 87.037 | 1.00 | 63.47 | A | O |
| ATOM | 1090 | NE2 | GLN | A | 149 | 28.675 | 39.618 | 85.975 | 1.00 | 58.60 | A | N |
| ATOM | 1091 | C | GLN | A | 149 | 33.575 | 38.967 | 85.006 | 1.00 | 42.65 | A | C |
| ATOM | 1092 | O | GLN | A | 149 | 34.141 | 39.989 | 84.615 | 1.00 | 43.03 | A | O |
| ATOM | 1093 | N | ASN | A | 150 | 33.999 | 38.247 | 86.046 | 1.00 | 41.42 | A | N |
| ATOM | 1094 | CA | ASN | A | 150 | 35.180 | 38.633 | 86.818 | 1.00 | 39.78 | A | C |
| ATOM | 1095 | CB | ASN | A | 150 | 35.244 | 37.866 | 88.134 | 1.00 | 42.17 | A | C |
| ATOM | 1096 | CG | ASN | A | 150 | 34.147 | 38.277 | 89.077 | 1.00 | 44.40 | A | C |
| ATOM | 1097 | OD1 | ASN | A | 150 | 33.314 | 37.465 | 89.478 | 1.00 | 45.18 | A | O |
| ATOM | 1098 | ND2 | ASN | A | 150 | 34.126 | 39.554 | 89.425 | 1.00 | 44.11 | A | N |
| ATOM | 1099 | C | ASN | A | 150 | 36.447 | 38.417 | 86.033 | 1.00 | 38.33 | A | C |
| ATOM | 1100 | O | ASN | A | 150 | 37.355 | 39.244 | 86.076 | 1.00 | 37.38 | A | O |
| ATOM | 1101 | N | ILE | A | 151 | 36.504 | 37.303 | 85.311 | 1.00 | 35.57 | A | N |
| ATOM | 1102 | CA | ILE | A | 151 | 37.656 | 37.004 | 84.482 | 1.00 | 32.84 | A | C |
| ATOM | 1103 | CB | ILE | A | 151 | 37.497 | 35.624 | 83.803 | 1.00 | 31.41 | A | C |
| ATOM | 1104 | CG2 | ILE | A | 151 | 38.548 | 35.450 | 82.718 | 1.00 | 30.47 | A | C |
| ATOM | 1105 | CG1 | ILE | A | 151 | 37.606 | 34.519 | 84.864 | 1.00 | 32.06 | A | C |
| ATOM | 1106 | CD1 | ILE | A | 151 | 37.323 | 33.084 | 84.365 | 1.00 | 25.62 | A | C |
| ATOM | 1107 | C | ILE | A | 151 | 37.739 | 38.108 | 83.428 | 1.00 | 33.12 | A | C |
| ATOM | 1108 | O | ILE | A | 151 | 38.786 | 38.716 | 83.216 | 1.00 | 31.31 | A | O |
| ATOM | 1109 | N | GLN | A | 152 | 36.606 | 38.382 | 82.797 | 1.00 | 32.77 | A | N |
| ATOM | 1110 | CA | GLN | A | 152 | 36.531 | 39.407 | 81.767 | 1.00 | 34.21 | A | C |
| ATOM | 1111 | CB | GLN | A | 152 | 35.103 | 39.506 | 81.235 | 1.00 | 34.45 | A | C |
| ATOM | 1112 | CG | GLN | A | 152 | 34.972 | 40.263 | 79.931 | 1.00 | 35.10 | A | C |
| ATOM | 1113 | CD | GLN | A | 152 | 35.670 | 39.554 | 78.782 | 1.00 | 37.09 | A | C |
| ATOM | 1114 | OE1 | GLN | A | 152 | 35.766 | 38.323 | 78.761 | 1.00 | 33.50 | A | O |
| ATOM | 1115 | NE2 | GLN | A | 152 | 36.142 | 40.328 | 77.810 | 1.00 | 34.10 | A | N |
| ATOM | 1116 | C | GLN | A | 152 | 36.953 | 40.767 | 82.301 | 1.00 | 33.89 | A | C |
| ATOM | 1117 | O | GLN | A | 152 | 37.683 | 41.506 | 81.642 | 1.00 | 33.26 | A | O |
| ATOM | 1118 | N | TYR | A | 153 | 36.482 | 41.095 | 83.497 | 1.00 | 33.61 | A | N |
| ATOM | 1119 | CA | TYR | A | 153 | 36.796 | 42.373 | 84.114 | 1.00 | 32.84 | A | C |
| ATOM | 1120 | CB | TYR | A | 153 | 36.030 | 42.523 | 85.429 | 1.00 | 32.46 | A | C |
| ATOM | 1121 | CG | TYR | A | 153 | 36.302 | 43.829 | 86.118 | 1.00 | 33.46 | A | C |
| ATOM | 1122 | CD1 | TYR | A | 153 | 35.691 | 45.003 | 85.685 | 1.00 | 35.76 | A | C |
| ATOM | 1123 | CE1 | TYR | A | 153 | 35.971 | 46.221 | 86.293 | 1.00 | 36.42 | A | C |
| ATOM | 1124 | CD2 | TYR | A | 153 | 37.204 | 43.904 | 87.178 | 1.00 | 34.35 | A | C |
| ATOM | 1125 | CE2 | TYR | A | 153 | 37.490 | 45.112 | 87.794 | 1.00 | 36.53 | A | C |
| ATOM | 1126 | CZ | TYR | A | 153 | 36.871 | 46.267 | 87.346 | 1.00 | 37.39 | A | C |
| ATOM | 1127 | OH | TYR | A | 153 | 37.152 | 47.464 | 87.960 | 1.00 | 42.52 | A | O |
| ATOM | 1128 | C | TYR | A | 153 | 38.292 | 42.478 | 84.375 | 1.00 | 32.36 | A | C |
| ATOM | 1129 | O | TYR | A | 153 | 38.938 | 43.447 | 83.963 | 1.00 | 33.54 | A | O |
| ATOM | 1130 | N | PHE | A | 154 | 38.841 | 41.469 | 85.048 | 1.00 | 29.67 | A | N |
| ATOM | 1131 | CA | PHE | A | 154 | 40.263 | 41.457 | 85.376 | 1.00 | 28.26 | A | C |
| ATOM | 1132 | CB | PHE | A | 154 | 40.592 | 40.304 | 86.348 | 1.00 | 27.10 | A | C |
| ATOM | 1133 | CG | PHE | A | 154 | 42.076 | 40.098 | 86.563 | 1.00 | 28.04 | A | C |
| ATOM | 1134 | CD1 | PHE | A | 154 | 42.770 | 39.130 | 85.846 | 1.00 | 28.04 | A | C |
| ATOM | 1135 | CD2 | PHE | A | 154 | 42.794 | 40.934 | 87.420 | 1.00 | 26.07 | A | C |
| ATOM | 1136 | CE1 | PHE | A | 154 | 44.164 | 39.003 | 85.974 | 1.00 | 27.91 | A | C |
| ATOM | 1137 | CE2 | PHE | A | 154 | 44.184 | 40.814 | 87.552 | 1.00 | 24.73 | A | C |
| ATOM | 1138 | CZ | PHE | A | 154 | 44.868 | 39.854 | 86.829 | 1.00 | 23.92 | A | C |
| ATOM | 1139 | C | PHE | A | 154 | 41.211 | 41.391 | 84.177 | 1.00 | 28.64 | A | C |
| ATOM | 1140 | O | PHE | A | 154 | 42.177 | 42.152 | 84.103 | 1.00 | 28.80 | A | O |
| ATOM | 1141 | N | LEU | A | 155 | 40.949 | 40.491 | 83.237 | 1.00 | 27.73 | A | N |
| ATOM | 1142 | CA | LEU | A | 155 | 41.834 | 40.366 | 82.094 | 1.00 | 26.44 | A | C |
| ATOM | 1143 | CB | LEU | A | 155 | 41.513 | 39.096 | 81.301 | 1.00 | 26.96 | A | C |
| ATOM | 1144 | CG | LEU | A | 155 | 42.078 | 37.819 | 81.950 | 1.00 | 28.23 | A | C |
| ATOM | 1145 | CD1 | LEU | A | 155 | 41.781 | 36.570 | 81.095 | 1.00 | 26.16 | A | C |
| ATOM | 1146 | CD2 | LEU | A | 155 | 43.580 | 37.993 | 82.113 | 1.00 | 25.98 | A | C |
| ATOM | 1147 | C | LEU | A | 155 | 41.839 | 41.584 | 81.183 | 1.00 | 26.88 | A | C |
| ATOM | 1148 | O | LEU | A | 155 | 42.869 | 41.917 | 80.610 | 1.00 | 26.57 | A | O |
| ATOM | 1149 | N | ASP | A | 156 | 40.710 | 42.263 | 81.035 | 1.00 | 26.74 | A | N |
| ATOM | 1150 | CA | ASP | A | 156 | 40.716 | 43.449 | 80.185 | 1.00 | 27.21 | A | C |
| ATOM | 1151 | CB | ASP | A | 156 | 39.308 | 44.035 | 80.050 | 1.00 | 26.81 | A | C |
| ATOM | 1152 | CG | ASP | A | 156 | 38.506 | 43.392 | 78.935 | 1.00 | 27.56 | A | C |
| ATOM | 1153 | OD1 | ASP | A | 156 | 39.111 | 42.749 | 78.052 | 1.00 | 28.05 | A | O |
| ATOM | 1154 | OD2 | ASP | A | 156 | 37.266 | 43.551 | 78.932 | 1.00 | 30.26 | A | O |
| ATOM | 1155 | C | ASP | A | 156 | 41.660 | 44.505 | 80.776 | 1.00 | 26.69 | A | C |
| ATOM | 1156 | O | ASP | A | 156 | 42.447 | 45.133 | 80.064 | 1.00 | 28.23 | A | O |
| ATOM | 1157 | N | ARG | A | 157 | 41.579 | 44.688 | 82.089 | 1.00 | 26.38 | A | N |
| ATOM | 1158 | CA | ARG | A | 157 | 42.421 | 45.664 | 82.761 | 1.00 | 26.56 | A | C |
| ATOM | 1159 | CB | ARG | A | 157 | 41.939 | 45.879 | 84.201 | 1.00 | 25.07 | A | C |
| ATOM | 1160 | CG | ARG | A | 157 | 40.567 | 46.565 | 84.279 | 1.00 | 27.56 | A | C |
| ATOM | 1161 | CD | ARG | A | 157 | 39.868 | 46.357 | 85.641 | 1.00 | 29.81 | A | C |
| ATOM | 1162 | NE | ARG | A | 157 | 40.573 | 47.029 | 86.724 | 1.00 | 29.81 | A | N |
| ATOM | 1163 | CZ | ARG | A | 157 | 40.602 | 48.348 | 86.890 | 1.00 | 31.11 | A | C |
| ATOM | 1164 | NH1 | ARG | A | 157 | 39.945 | 49.140 | 86.050 | 1.00 | 30.31 | A | N |
| ATOM | 1165 | NH2 | ARG | A | 157 | 41.335 | 48.876 | 87.860 | 1.00 | 30.68 | A | N |
| ATOM | 1166 | C | ARG | A | 157 | 43.878 | 45.240 | 82.742 | 1.00 | 26.64 | A | C |
| ATOM | 1167 | O | ARG | A | 157 | 44.745 | 46.026 | 82.380 | 1.00 | 28.00 | A | O |
| ATOM | 1168 | N | PHE | A | 158 | 44.141 | 43.992 | 83.106 | 1.00 | 27.05 | A | N |
| ATOM | 1169 | CA | PHE | A | 158 | 45.500 | 43.468 | 83.152 | 1.00 | 25.63 | A | C |
| ATOM | 1170 | CB | PHE | A | 158 | 45.476 | 42.015 | 83.620 | 1.00 | 26.85 | A | C |
| ATOM | 1171 | CG | PHE | A | 158 | 46.812 | 41.351 | 83.591 | 1.00 | 28.87 | A | C |
| ATOM | 1172 | CD1 | PHE | A | 158 | 47.764 | 41.628 | 84.565 | 1.00 | 29.91 | A | C |
| ATOM | 1173 | CD2 | PHE | A | 158 | 47.136 | 40.465 | 82.573 | 1.00 | 29.76 | A | C |
| ATOM | 1174 | CE1 | PHE | A | 158 | 49.025 | 41.027 | 84.528 | 1.00 | 28.41 | A | C |
| ATOM | 1175 | CE2 | PHE | A | 158 | 48.393 | 39.864 | 82.531 | 1.00 | 30.43 | A | C |
| ATOM | 1176 | CZ | PHE | A | 158 | 49.337 | 40.149 | 83.509 | 1.00 | 28.32 | A | C |
| ATOM | 1177 | C | PHE | A | 158 | 46.189 | 43.557 | 81.801 | 1.00 | 24.77 | A | C |
| ATOM | 1178 | O | PHE | A | 158 | 47.295 | 44.087 | 81.693 | 1.00 | 25.77 | A | O |
| ATOM | 1179 | N | TYR | A | 159 | 45.533 | 43.035 | 80.768 | 1.00 | 24.14 | A | N |
| ATOM | 1180 | CA | TYR | A | 159 | 46.105 | 43.064 | 79.435 | 1.00 | 21.16 | A | C |
| ATOM | 1181 | CB | TYR | A | 159 | 45.263 | 42.244 | 78.457 | 1.00 | 21.30 | A | C |
| ATOM | 1182 | CG | TYR | A | 159 | 45.383 | 40.739 | 78.623 | 1.00 | 22.89 | A | C |
| ATOM | 1183 | CD1 | TYR | A | 159 | 46.618 | 40.144 | 78.915 | 1.00 | 22.91 | A | C |
| ATOM | 1184 | CE1 | TYR | A | 159 | 46.742 | 38.757 | 79.024 | 1.00 | 24.80 | A | C |
| ATOM | 1185 | CD2 | TYR | A | 159 | 44.273 | 39.903 | 78.441 | 1.00 | 22.05 | A | C |
| ATOM | 1186 | CE2 | TYR | A | 159 | 44.394 | 38.514 | 78.540 | 1.00 | 20.79 | A | C |
| ATOM | 1187 | CZ | TYR | A | 159 | 45.632 | 37.951 | 78.832 | 1.00 | 22.61 | A | C |
| ATOM | 1188 | OH | TYR | A | 159 | 45.780 | 36.585 | 78.925 | 1.00 | 26.76 | A | O |
| ATOM | 1189 | C | TYR | A | 159 | 46.257 | 44.490 | 78.926 | 1.00 | 22.82 | A | C |
| ATOM | 1190 | O | TYR | A | 159 | 47.285 | 44.819 | 78.330 | 1.00 | 24.11 | A | O |
| ATOM | 1191 | N | LEU | A | 160 | 45.265 | 45.351 | 79.161 | 1.00 | 23.86 | A | N |
| ATOM | 1192 | CA | LEU | A | 160 | 45.382 | 46.739 | 78.689 | 1.00 | 26.24 | A | C |
| ATOM | 1193 | CB | LEU | A | 160 | 44.088 | 47.516 | 78.940 | 1.00 | 22.64 | A | C |
| ATOM | 1194 | CG | LEU | A | 160 | 44.110 | 48.974 | 78.457 | 1.00 | 25.35 | A | C |
| ATOM | 1195 | CD1 | LEU | A | 160 | 44.412 | 49.047 | 76.948 | 1.00 | 24.76 | A | C |
| ATOM | 1196 | CD2 | LEU | A | 160 | 42.759 | 49.631 | 78.764 | 1.00 | 24.58 | A | C |
| ATOM | 1197 | C | LEU | A | 160 | 46.562 | 47.430 | 79.393 | 1.00 | 25.01 | A | C |
| ATOM | 1198 | O | LEU | A | 160 | 47.349 | 48.170 | 78.777 | 1.00 | 24.80 | A | O |
| ATOM | 1199 | N | SER | A | 161 | 46.691 | 47.170 | 80.686 | 1.00 | 24.44 | A | N |
| ATOM | 1200 | CA | SER | A | 161 | 47.785 | 47.750 | 81.449 | 1.00 | 26.37 | A | C |
| ATOM | 1201 | CB | SER | A | 161 | 47.730 | 47.271 | 82.905 | 1.00 | 27.50 | A | C |
| ATOM | 1202 | OG | SER | A | 161 | 48.918 | 47.591 | 83.618 | 1.00 | 37.54 | A | O |
| ATOM | 1203 | C | SER | A | 161 | 49.097 | 47.322 | 80.803 | 1.00 | 25.63 | A | C |
| ATOM | 1204 | O | SER | A | 161 | 49.984 | 48.147 | 80.597 | 1.00 | 23.38 | A | O |
| ATOM | 1205 | N | ARG | A | 162 | 49.200 | 46.033 | 80.463 | 1.00 | 25.52 | A | N |
| ATOM | 1206 | CA | ARG | A | 162 | 50.415 | 45.479 | 79.854 | 1.00 | 24.75 | A | C |
| ATOM | 1207 | CB | ARG | A | 162 | 50.282 | 43.967 | 79.681 | 1.00 | 25.18 | A | C |
| ATOM | 1208 | CG | ARG | A | 162 | 51.393 | 43.344 | 78.858 | 1.00 | 25.56 | A | C |
| ATOM | 1209 | CD | ARG | A | 162 | 51.338 | 41.827 | 78.949 | 1.00 | 29.10 | A | C |
| ATOM | 1210 | NE | ARG | A | 162 | 52.479 | 41.183 | 78.299 | 1.00 | 30.26 | A | N |
| ATOM | 1211 | CZ | ARG | A | 162 | 52.494 | 40.789 | 77.030 | 1.00 | 32.77 | A | C |
| ATOM | 1212 | NH1 | ARG | A | 162 | 51.421 | 40.973 | 76.275 | 1.00 | 32.35 | A | N |
| ATOM | 1213 | NH2 | ARG | A | 162 | 53.575 | 40.200 | 76.518 | 1.00 | 31.82 | A | N |
| ATOM | 1214 | C | ARG | A | 162 | 50.722 | 46.119 | 78.512 | 1.00 | 24.97 | A | C |
| ATOM | 1215 | O | ARG | A | 162 | 51.870 | 46.474 | 78.222 | 1.00 | 21.84 | A | O |
| ATOM | 1216 | N | ILE | A | 163 | 49.691 | 46.250 | 77.685 | 1.00 | 24.44 | A | N |
| ATOM | 1217 | CA | ILE | A | 163 | 49.850 | 46.879 | 76.384 | 1.00 | 23.00 | A | C |
| ATOM | 1218 | CB | ILE | A | 163 | 48.473 | 47.035 | 75.675 | 1.00 | 23.72 | A | C |
| ATOM | 1219 | CG2 | ILE | A | 163 | 48.574 | 48.036 | 74.503 | 1.00 | 24.10 | A | C |
| ATOM | 1220 | CG1 | ILE | A | 163 | 47.984 | 45.662 | 75.196 | 1.00 | 23.24 | A | C |
| ATOM | 1221 | CD1 | ILE | A | 163 | 46.590 | 45.675 | 74.599 | 1.00 | 24.55 | A | C |
| ATOM | 1222 | C | ILE | A | 163 | 50.500 | 48.259 | 76.549 | 1.00 | 24.60 | A | C |
| ATOM | 1223 | O | ILE | A | 163 | 51.405 | 48.609 | 75.793 | 1.00 | 27.78 | A | O |
| ATOM | 1224 | N | SER | A | 164 | 50.053 | 49.035 | 77.539 | 1.00 | 24.39 | A | N |
| ATOM | 1225 | CA | SER | A | 164 | 50.596 | 50.379 | 77.771 | 1.00 | 24.64 | A | C |
| ATOM | 1226 | CB | SER | A | 164 | 49.724 | 51.158 | 78.755 | 1.00 | 23.33 | A | C |
| ATOM | 1227 | OG | SER | A | 164 | 49.939 | 50.722 | 80.081 | 1.00 | 25.45 | A | O |
| ATOM | 1228 | C | SER | A | 164 | 52.036 | 50.367 | 78.295 | 1.00 | 26.36 | A | C |
| ATOM | 1229 | O | SER | A | 164 | 52.831 | 51.259 | 77.967 | 1.00 | 24.05 | A | O |
| ATOM | 1230 | N | ILE | A | 165 | 52.375 | 49.373 | 79.114 | 1.00 | 24.93 | A | N |
| ATOM | 1231 | CA | ILE | A | 165 | 53.731 | 49.297 | 79.639 | 1.00 | 24.16 | A | C |
| ATOM | 1232 | CB | ILE | A | 165 | 53.850 | 48.233 | 80.758 | 1.00 | 23.95 | A | C |
| ATOM | 1233 | CG2 | ILE | A | 165 | 55.312 | 47.951 | 81.070 | 1.00 | 23.98 | A | C |
| ATOM | 1234 | CG1 | ILE | A | 165 | 53.138 | 48.735 | 82.016 | 1.00 | 23.22 | A | C |
| ATOM | 1235 | CD1 | ILE | A | 165 | 53.054 | 47.706 | 83.128 | 1.00 | 25.30 | A | C |
| ATOM | 1236 | C | ILE | A | 165 | 54.649 | 48.957 | 78.481 | 1.00 | 25.19 | A | C |
| ATOM | 1237 | O | ILE | A | 165 | 55.726 | 49.531 | 78.342 | 1.00 | 25.49 | A | O |
| ATOM | 1238 | N | ARG | A | 166 | 54.201 | 48.048 | 77.625 | 1.00 | 27.24 | A | N |
| ATOM | 1239 | CA | ARG | A | 166 | 55.000 | 47.644 | 76.481 | 1.00 | 28.89 | A | C |
| ATOM | 1240 | CB | ARG | A | 166 | 54.359 | 46.426 | 75.788 | 1.00 | 31.26 | A | C |
| ATOM | 1241 | CG | ARG | A | 166 | 54.757 | 45.076 | 76.435 | 1.00 | 35.54 | A | C |
| ATOM | 1242 | CD | ARG | A | 166 | 53.993 | 43.873 | 75.874 | 1.00 | 39.21 | A | C |
| ATOM | 1243 | NE | ARG | A | 166 | 54.071 | 43.796 | 74.420 | 1.00 | 47.65 | A | N |
| ATOM | 1244 | CZ | ARG | A | 166 | 55.074 | 43.246 | 73.744 | 1.00 | 54.40 | A | C |
| ATOM | 1245 | NH1 | ARG | A | 166 | 56.107 | 42.706 | 74.394 | 1.00 | 55.34 | A | N |
| ATOM | 1246 | NH2 | ARG | A | 166 | 55.045 | 43.244 | 72.413 | 1.00 | 56.16 | A | N |
| ATOM | 1247 | C | ARG | A | 166 | 55.186 | 48.817 | 75.519 | 1.00 | 29.28 | A | C |
| ATOM | 1248 | O | ARG | A | 166 | 56.230 | 48.929 | 74.878 | 1.00 | 28.10 | A | O |
| ATOM | 1249 | N | MET | A | 167 | 54.183 | 49.695 | 75.437 | 1.00 | 29.53 | A | N |
| ATOM | 1250 | CA | MET | A | 167 | 54.263 | 50.884 | 74.584 | 1.00 | 30.68 | A | C |
| ATOM | 1251 | CB | MET | A | 167 | 52.973 | 51.706 | 74.646 | 1.00 | 31.70 | A | C |
| ATOM | 1252 | CG | MET | A | 167 | 52.220 | 51.846 | 73.331 | 1.00 | 35.97 | A | C |
| ATOM | 1253 | SD | MET | A | 167 | 53.125 | 52.613 | 71.979 | 1.00 | 34.92 | A | S |
| ATOM | 1254 | CE | MET | A | 167 | 52.718 | 54.278 | 72.140 | 1.00 | 38.19 | A | C |
| ATOM | 1255 | C | MET | A | 167 | 55.400 | 51.758 | 75.104 | 1.00 | 29.32 | A | C |
| ATOM | 1256 | O | MET | A | 167 | 56.324 | 52.087 | 74.366 | 1.00 | 28.19 | A | O |
| ATOM | 1257 | N | LEU | A | 168 | 55.328 | 52.138 | 76.377 | 1.00 | 29.36 | A | N |
| ATOM | 1258 | CA | LEU | A | 168 | 56.373 | 52.981 | 76.945 | 1.00 | 30.34 | A | C |
| ATOM | 1259 | CB | LEU | A | 168 | 56.182 | 53.147 | 78.456 | 1.00 | 28.43 | A | C |
| ATOM | 1260 | CG | LEU | A | 168 | 54.871 | 53.840 | 78.845 | 1.00 | 32.34 | A | C |
| ATOM | 1261 | CD1 | LEU | A | 168 | 54.865 | 54.110 | 80.334 | 1.00 | 27.20 | A | C |
| ATOM | 1262 | CD2 | LEU | A | 168 | 54.704 | 55.141 | 78.076 | 1.00 | 29.97 | A | C |
| ATOM | 1263 | C | LEU | A | 168 | 57.748 | 52.402 | 76.659 | 1.00 | 29.08 | A | C |
| ATOM | 1264 | O | LEU | A | 168 | 58.606 | 53.081 | 76.104 | 1.00 | 30.06 | A | O |
| ATOM | 1265 | N | ILE | A | 169 | 57.940 | 51.138 | 77.019 | 1.00 | 29.30 | A | N |
| ATOM | 1266 | CA | ILE | A | 169 | 59.212 | 50.456 | 76.811 | 1.00 | 30.15 | A | C |
| ATOM | 1267 | CB | ILE | A | 169 | 59.149 | 49.021 | 77.354 | 1.00 | 27.37 | A | C |
| ATOM | 1268 | CG2 | ILE | A | 169 | 60.359 | 48.236 | 76.888 | 1.00 | 28.76 | A | C |
| ATOM | 1269 | CG1 | ILE | A | 169 | 59.036 | 49.063 | 78.877 | 1.00 | 27.34 | A | C |
| ATOM | 1270 | CD1 | ILE | A | 169 | 59.015 | 47.709 | 79.522 | 1.00 | 30.20 | A | C |
| ATOM | 1271 | C | ILE | A | 169 | 59.668 | 50.416 | 75.353 | 1.00 | 30.68 | A | C |
| ATOM | 1272 | O | ILE | A | 169 | 60.834 | 50.671 | 75.055 | 1.00 | 31.95 | A | O |
| ATOM | 1273 | N | ASN | A | 170 | 58.766 | 50.081 | 74.440 | 1.00 | 32.33 | A | N |
| ATOM | 1274 | CA | ASN | A | 170 | 59.144 | 50.033 | 73.032 | 1.00 | 35.99 | A | C |
| ATOM | 1275 | CB | ASN | A | 170 | 57.997 | 49.512 | 72.163 | 1.00 | 37.97 | A | C |
| ATOM | 1276 | CG | ASN | A | 170 | 57.757 | 48.035 | 72.355 | 1.00 | 43.03 | A | C |
| ATOM | 1277 | OD1 | ASN | A | 170 | 58.703 | 47.257 | 72.503 | 1.00 | 45.81 | A | O |
| ATOM | 1278 | ND2 | ASN | A | 170 | 56.488 | 47.632 | 72.344 | 1.00 | 44.83 | A | N |
| ATOM | 1279 | C | ASN | A | 170 | 59.564 | 51.403 | 72.532 | 1.00 | 34.93 | A | C |
| ATOM | 1280 | O | ASN | A | 170 | 60.514 | 51.523 | 71.762 | 1.00 | 36.11 | A | O |
| ATOM | 1281 | N | GLN | A | 171 | 58.847 | 52.433 | 72.964 | 1.00 | 33.56 | A | N |
| ATOM | 1282 | CA | GLN | A | 171 | 59.167 | 53.790 | 72.562 | 1.00 | 34.38 | A | C |
| ATOM | 1283 | CB | GLN | A | 171 | 58.161 | 54.768 | 73.164 | 1.00 | 34.69 | A | C |
| ATOM | 1284 | CG | GLN | A | 171 | 56.875 | 54.846 | 72.366 | 1.00 | 36.86 | A | C |
| ATOM | 1285 | CD | GLN | A | 171 | 57.143 | 55.182 | 70.905 | 1.00 | 38.74 | A | C |
| ATOM | 1286 | OE1 | GLN | A | 171 | 57.544 | 56.299 | 70.579 | 1.00 | 42.08 | A | O |
| ATOM | 1287 | NE2 | GLN | A | 171 | 56.938 | 54.211 | 70.024 | 1.00 | 35.80 | A | N |
| ATOM | 1288 | C | GLN | A | 171 | 60.577 | 54.160 | 72.987 | 1.00 | 33.79 | A | C |
| ATOM | 1289 | O | GLN | A | 171 | 61.347 | 54.703 | 72.203 | 1.00 | 32.60 | A | O |
| ATOM | 1290 | N | HIS | A | 172 | 60.919 | 53.848 | 74.229 | 1.00 | 34.08 | A | N |
| ATOM | 1291 | CA | HIS | A | 172 | 62.244 | 54.156 | 74.732 | 1.00 | 35.43 | A | C |
| ATOM | 1292 | CB | HIS | A | 172 | 62.307 | 53.916 | 76.237 | 1.00 | 34.62 | A | C |
| ATOM | 1293 | CG | HIS | A | 172 | 63.555 | 54.439 | 76.870 | 1.00 | 37.41 | A | C |
| ATOM | 1294 | CD2 | HIS | A | 172 | 64.709 | 53.820 | 77.215 | 1.00 | 38.12 | A | C |
| ATOM | 1295 | ND1 | HIS | A | 172 | 63.734 | 55.774 | 77.161 | 1.00 | 38.84 | A | N |
| ATOM | 1296 | CE1 | HIS | A | 172 | 64.946 | 55.955 | 77.657 | 1.00 | 39.57 | A | C |
| ATOM | 1297 | NE2 | HIS | A | 172 | 65.558 | 54.785 | 77.700 | 1.00 | 38.47 | A | N |
| ATOM | 1298 | C | HIS | A | 172 | 63.360 | 53.345 | 74.049 | 1.00 | 36.83 | A | C |
| ATOM | 1299 | O | HIS | A | 172 | 64.356 | 53.917 | 73.596 | 1.00 | 38.85 | A | O |
| ATOM | 1300 | N | THR | A | 173 | 63.208 | 52.024 | 73.967 | 1.00 | 35.98 | A | N |
| ATOM | 1301 | CA | THR | A | 173 | 64.256 | 51.208 | 73.365 | 1.00 | 36.46 | A | C |
| ATOM | 1302 | CB | THR | A | 173 | 64.018 | 49.698 | 73.589 | 1.00 | 38.01 | A | C |
| ATOM | 1303 | OG1 | THR | A | 173 | 62.912 | 49.253 | 72.791 | 1.00 | 39.25 | A | O |
| ATOM | 1304 | CG2 | THR | A | 173 | 63.734 | 49.415 | 75.052 | 1.00 | 37.65 | A | C |
| ATOM | 1305 | C | THR | A | 173 | 64.459 | 51.439 | 71.874 | 1.00 | 36.13 | A | C |
| ATOM | 1306 | O | THR | A | 173 | 65.590 | 51.477 | 71.405 | 1.00 | 37.40 | A | O |
| ATOM | 1307 | N | LEU | A | 174 | 63.372 | 51.591 | 71.129 | 1.00 | 37.64 | A | N |
| ATOM | 1308 | CA | LEU | A | 174 | 63.461 | 51.805 | 69.688 | 1.00 | 40.04 | A | C |
| ATOM | 1309 | CB | LEU | A | 174 | 62.072 | 51.742 | 69.052 | 1.00 | 39.05 | A | C |
| ATOM | 1310 | CG | LEU | A | 174 | 61.394 | 50.387 | 68.867 | 1.00 | 39.89 | A | C |
| ATOM | 1311 | CD1 | LEU | A | 174 | 59.986 | 50.585 | 68.345 | 1.00 | 39.46 | A | C |
| ATOM | 1312 | CD2 | LEU | A | 174 | 62.190 | 49.573 | 67.880 | 1.00 | 39.13 | A | C |
| ATOM | 1313 | C | LEU | A | 174 | 64.095 | 53.136 | 69.327 | 1.00 | 42.77 | A | C |
| ATOM | 1314 | O | LEU | A | 174 | 64.850 | 53.237 | 68.357 | 1.00 | 43.99 | A | O |
| ATOM | 1315 | N | ILE | A | 175 | 63.777 | 54.164 | 70.100 | 1.00 | 45.08 | A | N |
| ATOM | 1316 | CA | ILE | A | 175 | 64.311 | 55.493 | 69.836 | 1.00 | 48.53 | A | C |
| ATOM | 1317 | CB | ILE | A | 175 | 63.399 | 56.575 | 70.437 | 1.00 | 50.19 | A | C |
| ATOM | 1318 | CG2 | ILE | A | 175 | 64.007 | 57.943 | 70.190 | 1.00 | 49.91 | A | C |
| ATOM | 1319 | CG1 | ILE | A | 175 | 62.007 | 56.483 | 69.805 | 1.00 | 50.22 | A | C |
| ATOM | 1320 | CD1 | ILE | A | 175 | 60.998 | 57.431 | 70.376 | 1.00 | 50.59 | A | C |
| ATOM | 1321 | C | ILE | A | 175 | 65.743 | 55.731 | 70.332 | 1.00 | 48.71 | A | C |
| ATOM | 1322 | O | ILE | A | 175 | 66.565 | 56.306 | 69.618 | 1.00 | 48.46 | A | O |
| ATOM | 1323 | N | PHE | A | 176 | 66.024 | 55.294 | 71.554 | 1.00 | 49.73 | A | N |
| ATOM | 1324 | CA | PHE | A | 176 | 67.339 | 55.478 | 72.130 | 1.00 | 51.87 | A | C |
| ATOM | 1325 | CB | PHE | A | 176 | 67.200 | 55.901 | 73.592 | 1.00 | 48.67 | A | C |
| ATOM | 1326 | CG | PHE | A | 176 | 66.397 | 57.148 | 73.747 | 1.00 | 47.09 | A | C |
| ATOM | 1327 | CD1 | PHE | A | 176 | 66.809 | 58.325 | 73.125 | 1.00 | 46.54 | A | C |
| ATOM | 1328 | CD2 | PHE | A | 176 | 65.190 | 57.134 | 74.435 | 1.00 | 46.64 | A | C |
| ATOM | 1329 | CE1 | PHE | A | 176 | 66.028 | 59.466 | 73.182 | 1.00 | 46.58 | A | C |
| ATOM | 1330 | CE2 | PHE | A | 176 | 64.399 | 58.267 | 74.500 | 1.00 | 46.29 | A | C |
| ATOM | 1331 | CZ | PHE | A | 176 | 64.817 | 59.438 | 73.871 | 1.00 | 46.88 | A | C |
| ATOM | 1332 | C | PHE | A | 176 | 68.212 | 54.265 | 71.930 | 1.00 | 53.84 | A | C |
| ATOM | 1333 | O | PHE | A | 176 | 68.705 | 53.634 | 72.861 | 1.00 | 55.08 | A | O |
| ATOM | 1334 | N | ASP | A | 177 | 68.377 | 53.959 | 70.651 | 1.00 | 57.45 | A | N |
| ATOM | 1335 | CA | ASP | A | 177 | 69.201 | 52.859 | 70.179 | 1.00 | 60.70 | A | C |
| ATOM | 1336 | CB | ASP | A | 177 | 68.648 | 52.364 | 68.830 | 1.00 | 63.72 | A | C |
| ATOM | 1337 | CG | ASP | A | 177 | 68.556 | 53.479 | 67.783 | 1.00 | 67.23 | A | C |
| ATOM | 1338 | OD1 | ASP | A | 177 | 68.578 | 54.677 | 68.147 | 1.00 | 69.45 | A | O |
| ATOM | 1339 | OD2 | ASP | A | 177 | 68.444 | 53.148 | 66.588 | 1.00 | 69.37 | A | O |
| ATOM | 1340 | C | ASP | A | 177 | 70.629 | 53.409 | 70.023 | 1.00 | 61.47 | A | C |
| ATOM | 1341 | O | ASP | A | 177 | 71.619 | 52.707 | 70.224 | 1.00 | 63.61 | A | O |
| ATOM | 1342 | N | PRO | A | 185 | 64.595 | 61.251 | 61.172 | 1.00 | 58.91 | A | N |
| ATOM | 1343 | CD | PRO | A | 185 | 64.764 | 61.745 | 62.552 | 1.00 | 60.72 | A | C |
| ATOM | 1344 | CA | PRO | A | 185 | 63.982 | 59.912 | 61.233 | 1.00 | 58.58 | A | C |
| ATOM | 1345 | CB | PRO | A | 185 | 64.357 | 59.367 | 62.648 | 1.00 | 59.23 | A | C |
| ATOM | 1346 | CG | PRO | A | 185 | 65.338 | 60.450 | 63.258 | 1.00 | 61.27 | A | C |
| ATOM | 1347 | C | PRO | A | 185 | 62.475 | 60.116 | 61.095 | 1.00 | 57.84 | A | C |
| ATOM | 1348 | O | PRO | A | 185 | 61.807 | 60.880 | 61.826 | 1.00 | 55.87 | A | O |
| ATOM | 1349 | N | LYS | A | 186 | 61.950 | 59.464 | 60.080 | 1.00 | 54.03 | A | N |
| ATOM | 1350 | CA | LYS | A | 186 | 60.504 | 59.556 | 59.803 | 1.00 | 52.74 | A | C |
| ATOM | 1351 | CB | LYS | A | 186 | 60.275 | 59.069 | 58.383 | 1.00 | 51.96 | A | C |
| ATOM | 1352 | CG | LYS | A | 186 | 58.942 | 58.340 | 58.082 | 1.00 | 56.17 | A | C |
| ATOM | 1353 | CD | LYS | A | 186 | 57.699 | 59.129 | 58.515 | 1.00 | 58.61 | A | C |
| ATOM | 1354 | CE | LYS | A | 186 | 56.551 | 59.002 | 57.500 | 1.00 | 59.64 | A | C |
| ATOM | 1355 | NZ | LYS | A | 186 | 55.949 | 57.638 | 57.346 | 1.00 | 59.95 | A | N |
| ATOM | 1356 | C | LYS | A | 186 | 59.512 | 58.866 | 60.816 | 1.00 | 50.04 | A | C |
| ATOM | 1357 | O | LYS | A | 186 | 58.403 | 59.373 | 61.160 | 1.00 | 48.38 | A | O |
| ATOM | 1358 | N | HIS | A | 187 | 59.863 | 57.672 | 61.255 | 1.00 | 46.40 | A | N |
| ATOM | 1359 | CA | HIS | A | 187 | 59.064 | 57.036 | 62.291 | 1.00 | 43.77 | A | C |
| ATOM | 1360 | CB | HIS | A | 187 | 59.154 | 55.525 | 62.171 | 1.00 | 42.82 | A | C |
| ATOM | 1361 | CG | HIS | A | 187 | 58.183 | 54.932 | 61.196 | 1.00 | 39.81 | A | C |
| ATOM | 1362 | CD2 | HIS | A | 187 | 57.222 | 53.997 | 61.377 | 1.00 | 39.24 | A | C |
| ATOM | 1363 | ND1 | HIS | A | 187 | 58.198 | 55.203 | 59.843 | 1.00 | 40.74 | A | N |
| ATOM | 1364 | CE1 | HIS | A | 187 | 57.296 | 54.450 | 59.233 | 1.00 | 38.84 | A | C |
| ATOM | 1365 | NE2 | HIS | A | 187 | 56.692 | 53.708 | 60.144 | 1.00 | 39.63 | A | N |
| ATOM | 1366 | C | HIS | A | 187 | 59.532 | 57.466 | 63.709 | 1.00 | 41.62 | A | C |
| ATOM | 1367 | O | HIS | A | 187 | 60.726 | 57.658 | 63.974 | 1.00 | 43.48 | A | O |
| ATOM | 1368 | N | ILE | A | 188 | 58.593 | 57.622 | 64.633 | 1.00 | 38.64 | A | N |
| ATOM | 1369 | CA | ILE | A | 188 | 58.996 | 57.987 | 65.987 | 1.00 | 35.68 | A | C |
| ATOM | 1370 | CB | ILE | A | 188 | 58.182 | 59.170 | 66.550 | 1.00 | 35.45 | A | C |
| ATOM | 1371 | CG2 | ILE | A | 188 | 58.712 | 59.559 | 67.905 | 1.00 | 33.47 | A | C |
| ATOM | 1372 | CG1 | ILE | A | 188 | 58.236 | 60.365 | 65.593 | 1.00 | 36.32 | A | C |
| ATOM | 1373 | CD1 | ILE | A | 188 | 59.626 | 60.838 | 65.235 | 1.00 | 34.39 | A | C |
| ATOM | 1374 | C | ILE | A | 188 | 58.758 | 56.750 | 66.842 | 1.00 | 35.26 | A | C |
| ATOM | 1375 | O | ILE | A | 188 | 57.744 | 56.633 | 67.534 | 1.00 | 35.45 | A | O |
| ATOM | 1376 | N | GLY | A | 189 | 59.701 | 55.815 | 66.757 | 1.00 | 34.10 | A | N |
| ATOM | 1377 | CA | GLY | A | 189 | 59.589 | 54.569 | 67.491 | 1.00 | 32.82 | A | C |
| ATOM | 1378 | C | GLY | A | 189 | 58.578 | 53.699 | 66.769 | 1.00 | 32.44 | A | C |
| ATOM | 1379 | O | GLY | A | 189 | 58.725 | 53.442 | 65.574 | 1.00 | 31.65 | A | O |
| ATOM | 1380 | N | SER | A | 190 | 57.547 | 53.255 | 67.483 | 1.00 | 31.04 | A | N |
| ATOM | 1381 | CA | SER | A | 190 | 56.510 | 52.427 | 66.879 | 1.00 | 30.78 | A | C |
| ATOM | 1382 | CB | SER | A | 190 | 55.921 | 51.476 | 67.917 | 1.00 | 29.87 | A | C |
| ATOM | 1383 | OG | SER | A | 190 | 55.136 | 52.176 | 68.863 | 1.00 | 31.85 | A | O |
| ATOM | 1384 | C | SER | A | 190 | 55.390 | 53.297 | 66.293 | 1.00 | 31.76 | A | C |
| ATOM | 1385 | O | SER | A | 190 | 54.413 | 52.781 | 65.746 | 1.00 | 30.53 | A | O |
| ATOM | 1386 | N | ILE | A | 191 | 55.538 | 54.616 | 66.408 | 1.00 | 31.27 | A | N |
| ATOM | 1387 | CA | ILE | A | 191 | 54.532 | 55.549 | 65.895 | 1.00 | 29.89 | A | C |
| ATOM | 1388 | CB | ILE | A | 191 | 54.371 | 56.758 | 66.828 | 1.00 | 30.54 | A | C |
| ATOM | 1389 | CG2 | ILE | A | 191 | 53.485 | 57.821 | 66.171 | 1.00 | 27.37 | A | C |
| ATOM | 1390 | CG1 | ILE | A | 191 | 53.805 | 56.295 | 68.171 | 1.00 | 28.55 | A | C |
| ATOM | 1391 | CD1 | ILE | A | 191 | 53.896 | 57.334 | 69.238 | 1.00 | 27.01 | A | C |
| ATOM | 1392 | C | ILE | A | 191 | 54.857 | 56.098 | 64.516 | 1.00 | 29.45 | A | C |
| ATOM | 1393 | O | ILE | A | 191 | 55.964 | 56.568 | 64.274 | 1.00 | 30.78 | A | O |
| ATOM | 1394 | N | ASP | A | 192 | 53.885 | 56.035 | 63.615 | 1.00 | 29.46 | A | N |
| ATOM | 1395 | CA | ASP | A | 192 | 54.046 | 56.569 | 62.267 | 1.00 | 29.61 | A | C |
| ATOM | 1396 | CB | ASP | A | 192 | 53.584 | 55.561 | 61.229 | 1.00 | 30.15 | A | C |
| ATOM | 1397 | CG | ASP | A | 192 | 53.855 | 56.020 | 59.820 | 1.00 | 29.77 | A | C |
| ATOM | 1398 | OD1 | ASP | A | 192 | 54.079 | 57.234 | 59.622 | 1.00 | 32.34 | A | O |
| ATOM | 1399 | OD2 | ASP | A | 192 | 53.831 | 55.167 | 58.913 | 1.00 | 28.84 | A | O |
| ATOM | 1400 | C | ASP | A | 192 | 53.127 | 57.781 | 62.225 | 1.00 | 31.49 | A | C |
| ATOM | 1401 | O | ASP | A | 192 | 51.908 | 57.635 | 62.256 | 1.00 | 30.27 | A | O |
| ATOM | 1402 | N | PRO | A | 193 | 53.701 | 58.996 | 62.162 | 1.00 | 32.14 | A | N |
| ATOM | 1403 | CD | PRO | A | 193 | 55.134 | 59.317 | 62.051 | 1.00 | 31.96 | A | C |
| ATOM | 1404 | CA | PRO | A | 193 | 52.894 | 60.216 | 62.125 | 1.00 | 32.73 | A | C |
| ATOM | 1405 | CB | PRO | A | 193 | 53.920 | 61.304 | 62.418 | 1.00 | 31.53 | A | C |
| ATOM | 1406 | CG | PRO | A | 193 | 55.110 | 60.798 | 61.711 | 1.00 | 32.37 | A | C |
| ATOM | 1407 | C | PRO | A | 193 | 52.201 | 60.400 | 60.782 | 1.00 | 33.76 | A | C |
| ATOM | 1408 | O | PRO | A | 193 | 51.430 | 61.344 | 60.593 | 1.00 | 32.65 | A | O |
| ATOM | 1409 | N | ASN | A | 194 | 52.477 | 59.483 | 59.857 | 1.00 | 35.19 | A | N |
| ATOM | 1410 | CA | ASN | A | 194 | 51.888 | 59.530 | 58.524 | 1.00 | 38.12 | A | C |
| ATOM | 1411 | CB | ASN | A | 194 | 52.890 | 60.110 | 57.530 | 1.00 | 42.89 | A | C |
| ATOM | 1412 | CG | ASN | A | 194 | 52.829 | 61.621 | 57.474 | 1.00 | 48.79 | A | C |
| ATOM | 1413 | OD1 | ASN | A | 194 | 51.865 | 62.194 | 56.949 | 1.00 | 51.53 | A | O |
| ATOM | 1414 | ND2 | ASN | A | 194 | 53.843 | 62.280 | 58.034 | 1.00 | 48.37 | A | N |
| ATOM | 1415 | C | ASN | A | 194 | 51.407 | 58.176 | 58.039 | 1.00 | 37.75 | A | C |
| ATOM | 1416 | O | ASN | A | 194 | 51.528 | 57.843 | 56.859 | 1.00 | 37.74 | A | O |
| ATOM | 1417 | N | CYS | A | 195 | 50.830 | 57.418 | 58.962 | 1.00 | 35.99 | A | N |
| ATOM | 1418 | CA | CYS | A | 195 | 50.321 | 56.089 | 58.689 | 1.00 | 33.74 | A | C |
| ATOM | 1419 | CB | CYS | A | 195 | 49.801 | 55.484 | 59.993 | 1.00 | 32.39 | A | C |
| ATOM | 1420 | SG | CYS | A | 195 | 49.102 | 53.845 | 59.822 | 1.00 | 31.31 | A | S |
| ATOM | 1421 | C | CYS | A | 195 | 49.219 | 56.066 | 57.634 | 1.00 | 33.44 | A | C |
| ATOM | 1422 | O | CYS | A | 195 | 48.174 | 56.697 | 57.796 | 1.00 | 33.26 | A | O |
| ATOM | 1423 | N | ASN | A | 196 | 49.467 | 55.343 | 56.547 | 1.00 | 33.11 | A | N |
| ATOM | 1424 | CA | ASN | A | 196 | 48.482 | 55.210 | 55.485 | 1.00 | 32.89 | A | C |
| ATOM | 1425 | CB | ASN | A | 196 | 49.165 | 55.038 | 54.129 | 1.00 | 35.02 | A | C |
| ATOM | 1426 | CG | ASN | A | 196 | 48.175 | 54.731 | 53.015 | 1.00 | 37.38 | A | C |
| ATOM | 1427 | OD1 | ASN | A | 196 | 47.512 | 53.698 | 53.025 | 1.00 | 40.58 | A | O |
| ATOM | 1428 | ND2 | ASN | A | 196 | 48.072 | 55.629 | 52.053 | 1.00 | 41.43 | A | N |
| ATOM | 1429 | C | ASN | A | 196 | 47.684 | 53.964 | 55.839 | 1.00 | 31.67 | A | C |
| ATOM | 1430 | O | ASN | A | 196 | 48.152 | 52.837 | 55.676 | 1.00 | 31.90 | A | O |
| ATOM | 1431 | N | VAL | A | 197 | 46.474 | 54.176 | 56.332 | 1.00 | 30.01 | A | N |
| ATOM | 1432 | CA | VAL | A | 197 | 45.625 | 53.078 | 56.755 | 1.00 | 29.93 | A | C |
| ATOM | 1433 | CB | VAL | A | 197 | 44.268 | 53.620 | 57.225 | 1.00 | 28.46 | A | C |
| ATOM | 1434 | CG1 | VAL | A | 197 | 43.353 | 52.480 | 57.672 | 1.00 | 26.16 | A | C |
| ATOM | 1435 | CG2 | VAL | A | 197 | 44.509 | 54.595 | 58.370 | 1.00 | 27.40 | A | C |
| ATOM | 1436 | C | VAL | A | 197 | 45.434 | 51.963 | 55.738 | 1.00 | 28.66 | A | C |
| ATOM | 1437 | O | VAL | A | 197 | 45.615 | 50.788 | 56.076 | 1.00 | 30.13 | A | O |
| ATOM | 1438 | N | SER | A | 198 | 45.088 | 52.318 | 54.505 | 1.00 | 27.89 | A | N |
| ATOM | 1439 | CA | SER | A | 198 | 44.885 | 51.324 | 53.456 | 1.00 | 29.64 | A | C |
| ATOM | 1440 | CB | SER | A | 198 | 44.486 | 51.993 | 52.146 | 1.00 | 29.72 | A | C |
| ATOM | 1441 | OG | SER | A | 198 | 43.127 | 52.374 | 52.206 | 1.00 | 31.73 | A | O |
| ATOM | 1442 | C | SER | A | 198 | 46.102 | 50.450 | 53.230 | 1.00 | 30.09 | A | C |
| ATOM | 1443 | O | SER | A | 198 | 45.961 | 49.271 | 52.923 | 1.00 | 31.66 | A | O |
| ATOM | 1444 | N | GLU | A | 199 | 47.294 | 51.015 | 53.380 | 1.00 | 29.68 | A | N |
| ATOM | 1445 | CA | GLU | A | 199 | 48.513 | 50.232 | 53.200 | 1.00 | 31.66 | A | C |
| ATOM | 1446 | CB | GLU | A | 199 | 49.747 | 51.140 | 53.228 | 1.00 | 34.13 | A | C |
| ATOM | 1447 | CG | GLU | A | 199 | 51.090 | 50.407 | 53.116 | 1.00 | 39.72 | A | C |
| ATOM | 1448 | CD | GLU | A | 199 | 51.154 | 49.427 | 51.937 | 1.00 | 44.89 | A | C |
| ATOM | 1449 | OE1 | GLU | A | 199 | 50.394 | 49.607 | 50.952 | 1.00 | 48.11 | A | O |
| ATOM | 1450 | OE2 | GLU | A | 199 | 51.972 | 48.475 | 51.993 | 1.00 | 45.62 | A | O |
| ATOM | 1451 | C | GLU | A | 199 | 48.620 | 49.167 | 54.296 | 1.00 | 31.05 | A | C |
| ATOM | 1452 | O | GLU | A | 199 | 49.070 | 48.043 | 54.045 | 1.00 | 31.15 | A | O |
| ATOM | 1453 | N | VAL | A | 200 | 48.204 | 49.507 | 55.510 | 1.00 | 28.80 | A | N |
| ATOM | 1454 | CA | VAL | A | 200 | 48.263 | 48.536 | 56.590 | 1.00 | 28.10 | A | C |
| ATOM | 1455 | CB | VAL | A | 200 | 48.082 | 49.206 | 57.948 | 1.00 | 27.68 | A | C |
| ATOM | 1456 | CG1 | VAL | A | 200 | 48.144 | 48.155 | 59.042 | 1.00 | 26.16 | A | C |
| ATOM | 1457 | CG2 | VAL | A | 200 | 49.160 | 50.254 | 58.156 | 1.00 | 23.36 | A | C |
| ATOM | 1458 | C | VAL | A | 200 | 47.179 | 47.472 | 56.392 | 1.00 | 30.44 | A | C |
| ATOM | 1459 | O | VAL | A | 200 | 47.368 | 46.305 | 56.740 | 1.00 | 30.02 | A | O |
| ATOM | 1460 | N | VAL | A | 201 | 46.041 | 47.881 | 55.834 | 1.00 | 29.96 | A | N |
| ATOM | 1461 | CA | VAL | A | 201 | 44.961 | 46.948 | 55.562 | 1.00 | 29.79 | A | C |
| ATOM | 1462 | CB | VAL | A | 201 | 43.745 | 47.679 | 54.944 | 1.00 | 31.13 | A | C |
| ATOM | 1463 | CG1 | VAL | A | 201 | 42.698 | 46.672 | 54.476 | 1.00 | 26.84 | A | C |
| ATOM | 1464 | CG2 | VAL | A | 201 | 43.148 | 48.623 | 55.965 | 1.00 | 29.00 | A | C |
| ATOM | 1465 | C | VAL | A | 201 | 45.451 | 45.871 | 54.581 | 1.00 | 30.80 | A | C |
| ATOM | 1466 | O | VAL | A | 201 | 45.254 | 44.677 | 54.813 | 1.00 | 30.43 | A | O |
| ATOM | 1467 | N | LYS | A | 202 | 46.093 | 46.305 | 53.494 | 1.00 | 31.83 | A | N |
| ATOM | 1468 | CA | LYS | A | 202 | 46.614 | 45.394 | 52.468 | 1.00 | 33.31 | A | C |
| ATOM | 1469 | CB | LYS | A | 202 | 47.257 | 46.181 | 51.318 | 1.00 | 35.31 | A | C |
| ATOM | 1470 | CG | LYS | A | 202 | 46.306 | 47.140 | 50.631 | 1.00 | 41.15 | A | C |
| ATOM | 1471 | CD | LYS | A | 202 | 47.009 | 48.035 | 49.613 | 1.00 | 46.43 | A | C |
| ATOM | 1472 | CE | LYS | A | 202 | 46.066 | 49.125 | 49.102 | 1.00 | 49.82 | A | C |
| ATOM | 1473 | NZ | LYS | A | 202 | 46.733 | 50.046 | 48.136 | 1.00 | 53.95 | A | N |
| ATOM | 1474 | C | LYS | A | 202 | 47.640 | 44.438 | 53.056 | 1.00 | 31.77 | A | C |
| ATOM | 1475 | O | LYS | A | 202 | 47.575 | 43.230 | 52.812 | 1.00 | 31.65 | A | O |
| ATOM | 1476 | N | ASP | A | 203 | 48.582 | 44.983 | 53.825 | 1.00 | 28.73 | A | N |
| ATOM | 1477 | CA | ASP | A | 203 | 49.616 | 44.176 | 54.464 | 1.00 | 29.19 | A | C |
| ATOM | 1478 | CB | ASP | A | 203 | 50.482 | 45.035 | 55.400 | 1.00 | 30.40 | A | C |
| ATOM | 1479 | CG | ASP | A | 203 | 51.411 | 45.991 | 54.652 | 1.00 | 32.66 | A | C |
| ATOM | 1480 | OD1 | ASP | A | 203 | 51.458 | 45.964 | 53.401 | 1.00 | 33.78 | A | O |
| ATOM | 1481 | OD2 | ASP | A | 203 | 52.107 | 46.775 | 55.331 | 1.00 | 32.91 | A | O |
| ATOM | 1482 | C | ASP | A | 203 | 48.996 | 43.037 | 55.271 | 1.00 | 28.01 | A | C |
| ATOM | 1483 | O | ASP | A | 203 | 49.367 | 41.870 | 55.101 | 1.00 | 27.91 | A | O |
| ATOM | 1484 | N | ALA | A | 204 | 48.050 | 43.375 | 56.146 | 1.00 | 26.60 | A | N |
| ATOM | 1485 | CA | ALA | A | 204 | 47.396 | 42.365 | 56.980 | 1.00 | 26.20 | A | C |
| ATOM | 1486 | CB | ALA | A | 204 | 46.469 | 43.018 | 57.988 | 1.00 | 25.14 | A | C |
| ATOM | 1487 | C | ALA | A | 204 | 46.622 | 41.378 | 56.121 | 1.00 | 26.45 | A | C |
| ATOM | 1488 | O | ALA | A | 204 | 46.610 | 40.183 | 56.407 | 1.00 | 24.96 | A | O |
| ATOM | 1489 | N | TYR | A | 205 | 45.979 | 41.879 | 55.069 | 1.00 | 26.30 | A | N |
| ATOM | 1490 | CA | TYR | A | 205 | 45.237 | 41.009 | 54.172 | 1.00 | 28.59 | A | C |
| ATOM | 1491 | CB | TYR | A | 205 | 44.551 | 41.818 | 53.075 | 1.00 | 28.77 | A | C |
| ATOM | 1492 | CG | TYR | A | 205 | 44.026 | 40.954 | 51.950 | 1.00 | 30.86 | A | C |
| ATOM | 1493 | CD1 | TYR | A | 205 | 42.883 | 40.159 | 52.112 | 1.00 | 31.87 | A | C |
| ATOM | 1494 | CE1 | TYR | A | 205 | 42.427 | 39.331 | 51.079 | 1.00 | 29.68 | A | C |
| ATOM | 1495 | CD2 | TYR | A | 205 | 44.696 | 40.898 | 50.732 | 1.00 | 30.62 | A | C |
| ATOM | 1496 | CE2 | TYR | A | 205 | 44.253 | 40.075 | 49.700 | 1.00 | 30.59 | A | C |
| ATOM | 1497 | CZ | TYR | A | 205 | 43.118 | 39.299 | 49.875 | 1.00 | 30.66 | A | C |
| ATOM | 1498 | OH | TYR | A | 205 | 42.660 | 38.519 | 48.835 | 1.00 | 30.30 | A | O |
| ATOM | 1499 | C | TYR | A | 205 | 46.204 | 40.018 | 53.529 | 1.00 | 29.34 | A | C |
| ATOM | 1500 | O | TYR | A | 205 | 45.973 | 38.814 | 53.567 | 1.00 | 29.58 | A | O |
| ATOM | 1501 | N | ASP | A | 206 | 47.285 | 40.533 | 52.948 | 1.00 | 29.05 | A | N |
| ATOM | 1502 | CA | ASP | A | 206 | 48.281 | 39.690 | 52.294 | 1.00 | 30.98 | A | C |
| ATOM | 1503 | CB | ASP | A | 206 | 49.459 | 40.551 | 51.799 | 1.00 | 32.44 | A | C |
| ATOM | 1504 | CG | ASP | A | 206 | 49.083 | 41.438 | 50.605 | 1.00 | 33.88 | A | C |
| ATOM | 1505 | OD1 | ASP | A | 206 | 48.194 | 41.056 | 49.815 | 1.00 | 34.39 | A | O |
| ATOM | 1506 | OD2 | ASP | A | 206 | 49.688 | 42.515 | 50.438 | 1.00 | 35.73 | A | O |
| ATOM | 1507 | C | ASP | A | 206 | 48.785 | 38.550 | 53.192 | 1.00 | 30.88 | A | C |
| ATOM | 1508 | O | ASP | A | 206 | 48.915 | 37.414 | 52.740 | 1.00 | 30.50 | A | O |
| ATOM | 1509 | N | MET | A | 207 | 49.058 | 38.841 | 54.461 | 1.00 | 30.74 | A | N |
| ATOM | 1510 | CA | MET | A | 207 | 49.521 | 37.803 | 55.381 | 1.00 | 32.03 | A | C |
| ATOM | 1511 | CB | MET | A | 207 | 49.919 | 38.388 | 56.739 | 1.00 | 31.98 | A | C |
| ATOM | 1512 | CG | MET | A | 207 | 51.384 | 38.195 | 57.141 | 1.00 | 37.12 | A | C |
| ATOM | 1513 | SD | MET | A | 207 | 52.170 | 36.617 | 56.691 | 1.00 | 38.04 | A | S |
| ATOM | 1514 | CE | MET | A | 207 | 51.519 | 35.497 | 57.872 | 1.00 | 43.24 | A | C |
| ATOM | 1515 | C | MET | A | 207 | 48.420 | 36.779 | 55.625 | 1.00 | 32.37 | A | C |
| ATOM | 1516 | O | MET | A | 207 | 48.679 | 35.579 | 55.657 | 1.00 | 34.32 | A | O |
| ATOM | 1517 | N | ALA | A | 208 | 47.196 | 37.258 | 55.832 | 1.00 | 30.49 | A | N |
| ATOM | 1518 | CA | ALA | A | 208 | 46.076 | 36.363 | 56.077 | 1.00 | 30.32 | A | C |
| ATOM | 1519 | CB | ALA | A | 208 | 44.810 | 37.162 | 56.356 | 1.00 | 31.00 | A | C |
| ATOM | 1520 | C | ALA | A | 208 | 45.873 | 35.459 | 54.863 | 1.00 | 30.68 | A | C |
| ATOM | 1521 | O | ALA | A | 208 | 45.694 | 34.253 | 55.014 | 1.00 | 29.18 | A | O |
| ATOM | 1522 | N | LYS | A | 209 | 45.902 | 36.049 | 53.669 | 1.00 | 31.41 | A | N |
| ATOM | 1523 | CA | LYS | A | 209 | 45.737 | 35.303 | 52.423 | 1.00 | 34.96 | A | C |
| ATOM | 1524 | CB | LYS | A | 209 | 45.846 | 36.255 | 51.222 | 1.00 | 35.83 | A | C |
| ATOM | 1525 | CG | LYS | A | 209 | 45.964 | 35.567 | 49.862 | 1.00 | 36.05 | A | C |
| ATOM | 1526 | CD | LYS | A | 209 | 45.638 | 36.522 | 48.723 | 1.00 | 37.54 | A | C |
| ATOM | 1527 | CE | LYS | A | 209 | 46.202 | 36.040 | 47.390 | 1.00 | 41.64 | A | C |
| ATOM | 1528 | NZ | LYS | A | 209 | 46.014 | 34.574 | 47.157 | 1.00 | 46.57 | A | N |
| ATOM | 1529 | C | LYS | A | 209 | 46.776 | 34.185 | 52.310 | 1.00 | 36.74 | A | C |
| ATOM | 1530 | O | LYS | A | 209 | 46.444 | 33.040 | 51.982 | 1.00 | 36.94 | A | O |
| ATOM | 1531 | N | LEU | A | 210 | 48.031 | 34.515 | 52.602 | 1.00 | 38.12 | A | N |
| ATOM | 1532 | CA | LEU | A | 210 | 49.100 | 33.529 | 52.536 | 1.00 | 40.34 | A | C |
| ATOM | 1533 | CB | LEU | A | 210 | 50.406 | 34.116 | 53.060 | 1.00 | 43.51 | A | C |
| ATOM | 1534 | CG | LEU | A | 210 | 51.647 | 33.361 | 52.581 | 1.00 | 47.82 | A | C |
| ATOM | 1535 | CD1 | LEU | A | 210 | 51.921 | 33.748 | 51.130 | 1.00 | 48.53 | A | C |
| ATOM | 1536 | CD2 | LEU | A | 210 | 52.845 | 33.703 | 53.452 | 1.00 | 50.72 | A | C |
| ATOM | 1537 | C | LEU | A | 210 | 48.738 | 32.305 | 53.366 | 1.00 | 39.33 | A | C |
| ATOM | 1538 | O | LEU | A | 210 | 48.802 | 31.172 | 52.882 | 1.00 | 38.48 | A | O |
| ATOM | 1539 | N | LEU | A | 211 | 48.361 | 32.541 | 54.620 | 1.00 | 37.29 | A | N |
| ATOM | 1540 | CA | LEU | A | 211 | 47.992 | 31.449 | 55.511 | 1.00 | 37.08 | A | C |
| ATOM | 1541 | CB | LEU | A | 211 | 47.746 | 31.980 | 56.917 | 1.00 | 35.40 | A | C |
| ATOM | 1542 | CG | LEU | A | 211 | 48.978 | 32.511 | 57.650 | 1.00 | 36.63 | A | C |
| ATOM | 1543 | CD1 | LEU | A | 211 | 48.547 | 33.196 | 58.931 | 1.00 | 32.59 | A | C |
| ATOM | 1544 | CD2 | LEU | A | 211 | 49.938 | 31.368 | 57.946 | 1.00 | 37.61 | A | C |
| ATOM | 1545 | C | LEU | A | 211 | 46.744 | 30.734 | 55.011 | 1.00 | 38.19 | A | C |
| ATOM | 1546 | O | LEU | A | 211 | 46.677 | 29.504 | 54.994 | 1.00 | 38.48 | A | O |
| ATOM | 1547 | N | CYS | A | 212 | 45.759 | 31.521 | 54.601 | 1.00 | 39.29 | A | N |
| ATOM | 1548 | CA | CYS | A | 212 | 44.504 | 30.989 | 54.105 | 1.00 | 40.03 | A | C |
| ATOM | 1549 | CB | CYS | A | 212 | 43.588 | 32.141 | 53.687 | 1.00 | 38.86 | A | C |
| ATOM | 1550 | SG | CYS | A | 212 | 41.890 | 31.656 | 53.362 | 1.00 | 39.76 | A | S |
| ATOM | 1551 | C | CYS | A | 212 | 44.731 | 30.034 | 52.935 | 1.00 | 39.53 | A | C |
| ATOM | 1552 | O | CYS | A | 212 | 44.107 | 28.980 | 52.871 | 1.00 | 37.20 | A | O |
| ATOM | 1553 | N | ASP | A | 213 | 45.621 | 30.398 | 52.013 | 1.00 | 39.76 | A | N |
| ATOM | 1554 | CA | ASP | A | 213 | 45.901 | 29.529 | 50.870 | 1.00 | 41.42 | A | C |
| ATOM | 1555 | CB | ASP | A | 213 | 46.752 | 30.256 | 49.822 | 1.00 | 38.68 | A | C |
| ATOM | 1556 | CG | ASP | A | 213 | 45.974 | 31.317 | 49.067 | 1.00 | 39.28 | A | C |
| ATOM | 1557 | OD1 | ASP | A | 213 | 44.740 | 31.180 | 48.962 | 1.00 | 40.32 | A | O |
| ATOM | 1558 | OD2 | ASP | A | 213 | 46.590 | 32.279 | 48.564 | 1.00 | 38.43 | A | O |
| ATOM | 1559 | C | ASP | A | 213 | 46.600 | 28.225 | 51.274 | 1.00 | 42.87 | A | C |
| ATOM | 1560 | O | ASP | A | 213 | 46.333 | 27.172 | 50.696 | 1.00 | 44.66 | A | O |
| ATOM | 1561 | N | LYS | A | 214 | 47.490 | 28.290 | 52.261 | 1.00 | 42.70 | A | N |
| ATOM | 1562 | CA | LYS | A | 214 | 48.204 | 27.095 | 52.709 | 1.00 | 45.75 | A | C |
| ATOM | 1563 | CB | LYS | A | 214 | 49.266 | 27.440 | 53.760 | 1.00 | 45.65 | A | C |
| ATOM | 1564 | CG | LYS | A | 214 | 50.550 | 28.011 | 53.208 | 1.00 | 47.36 | A | C |
| ATOM | 1565 | CD | LYS | A | 214 | 51.292 | 26.987 | 52.376 | 1.00 | 46.95 | A | C |
| ATOM | 1566 | CE | LYS | A | 214 | 52.495 | 27.621 | 51.705 | 1.00 | 45.95 | A | C |
| ATOM | 1567 | NZ | LYS | A | 214 | 53.276 | 26.618 | 50.947 | 1.00 | 46.75 | A | N |
| ATOM | 1568 | C | LYS | A | 214 | 47.282 | 26.054 | 53.312 | 1.00 | 46.89 | A | C |
| ATOM | 1569 | O | LYS | A | 214 | 47.463 | 24.859 | 53.103 | 1.00 | 48.50 | A | O |
| ATOM | 1570 | N | TYR | A | 215 | 46.296 | 26.514 | 54.068 | 1.00 | 47.25 | A | N |
| ATOM | 1571 | CA | TYR | A | 215 | 45.370 | 25.620 | 54.739 | 1.00 | 48.59 | A | C |
| ATOM | 1572 | CB | TYR | A | 215 | 44.988 | 26.246 | 56.081 | 1.00 | 51.98 | A | C |
| ATOM | 1573 | CG | TYR | A | 215 | 44.174 | 25.363 | 56.995 | 1.00 | 58.76 | A | C |
| ATOM | 1574 | CD1 | TYR | A | 215 | 42.806 | 25.179 | 56.791 | 1.00 | 60.42 | A | C |
| ATOM | 1575 | CE1 | TYR | A | 215 | 42.054 | 24.361 | 57.642 | 1.00 | 62.32 | A | C |
| ATOM | 1576 | CD2 | TYR | A | 215 | 44.776 | 24.707 | 58.071 | 1.00 | 61.05 | A | C |
| ATOM | 1577 | CE2 | TYR | A | 215 | 44.033 | 23.885 | 58.928 | 1.00 | 62.75 | A | C |
| ATOM | 1578 | CZ | TYR | A | 215 | 42.674 | 23.715 | 58.709 | 1.00 | 63.28 | A | C |
| ATOM | 1579 | OH | TYR | A | 215 | 41.947 | 22.894 | 59.552 | 1.00 | 62.41 | A | O |
| ATOM | 1580 | C | TYR | A | 215 | 44.114 | 25.264 | 53.931 | 1.00 | 48.09 | A | C |
| ATOM | 1581 | O | TYR | A | 215 | 43.642 | 24.129 | 53.974 | 1.00 | 46.96 | A | O |
| ATOM | 1582 | N | TYR | A | 216 | 43.579 | 26.224 | 53.186 | 1.00 | 48.37 | A | N |
| ATOM | 1583 | CA | TYR | A | 216 | 42.371 | 25.976 | 52.412 | 1.00 | 48.50 | A | C |
| ATOM | 1584 | CB | TYR | A | 216 | 41.326 | 27.058 | 52.701 | 1.00 | 46.57 | A | C |
| ATOM | 1585 | CG | TYR | A | 216 | 40.838 | 27.092 | 54.128 | 1.00 | 44.54 | A | C |
| ATOM | 1586 | CD1 | TYR | A | 216 | 41.264 | 28.086 | 55.007 | 1.00 | 43.12 | A | C |
| ATOM | 1587 | CE1 | TYR | A | 216 | 40.802 | 28.133 | 56.318 | 1.00 | 44.19 | A | C |
| ATOM | 1588 | CD2 | TYR | A | 216 | 39.934 | 26.136 | 54.600 | 1.00 | 44.07 | A | C |
| ATOM | 1589 | CE2 | TYR | A | 216 | 39.465 | 26.173 | 55.912 | 1.00 | 44.10 | A | C |
| ATOM | 1590 | CZ | TYR | A | 216 | 39.901 | 27.175 | 56.766 | 1.00 | 46.03 | A | C |
| ATOM | 1591 | OH | TYR | A | 216 | 39.432 | 27.234 | 58.063 | 1.00 | 45.58 | A | O |
| ATOM | 1592 | C | TYR | A | 216 | 42.575 | 25.883 | 50.906 | 1.00 | 50.90 | A | C |
| ATOM | 1593 | O | TYR | A | 216 | 41.660 | 25.498 | 50.179 | 1.00 | 51.60 | A | O |
| ATOM | 1594 | N | MET | A | 217 | 43.758 | 26.239 | 50.425 | 1.00 | 53.58 | A | N |
| ATOM | 1595 | CA | MET | A | 217 | 44.005 | 26.173 | 48.991 | 1.00 | 57.62 | A | C |
| ATOM | 1596 | CB | MET | A | 217 | 44.078 | 24.704 | 48.562 | 1.00 | 62.74 | A | C |
| ATOM | 1597 | CG | MET | A | 217 | 45.207 | 23.949 | 49.276 | 1.00 | 69.55 | A | C |
| ATOM | 1598 | SD | MET | A | 217 | 45.178 | 22.145 | 49.107 | 1.00 | 79.42 | A | S |
| ATOM | 1599 | CE | MET | A | 217 | 44.258 | 21.657 | 50.585 | 1.00 | 77.33 | A | C |
| ATOM | 1600 | C | MET | A | 217 | 42.893 | 26.924 | 48.249 | 1.00 | 57.14 | A | C |
| ATOM | 1601 | O | MET | A | 217 | 42.407 | 26.500 | 47.194 | 1.00 | 57.74 | A | O |
| ATOM | 1602 | N | ALA | A | 218 | 42.507 | 28.052 | 48.839 | 1.00 | 54.63 | A | N |
| ATOM | 1603 | CA | ALA | A | 218 | 41.476 | 28.943 | 48.313 | 1.00 | 50.49 | A | C |
| ATOM | 1604 | CB | ALA | A | 218 | 40.146 | 28.218 | 48.215 | 1.00 | 50.86 | A | C |
| ATOM | 1605 | C | ALA | A | 218 | 41.369 | 30.124 | 49.284 | 1.00 | 46.69 | A | C |
| ATOM | 1606 | O | ALA | A | 218 | 41.541 | 29.955 | 50.493 | 1.00 | 46.11 | A | O |
| ATOM | 1607 | N | SER | A | 219 | 41.114 | 31.317 | 48.760 | 1.00 | 41.75 | A | N |
| ATOM | 1608 | CA | SER | A | 219 | 40.995 | 32.493 | 49.605 | 1.00 | 39.24 | A | C |
| ATOM | 1609 | CB | SER | A | 219 | 42.379 | 33.010 | 50.003 | 1.00 | 39.52 | A | C |
| ATOM | 1610 | OG | SER | A | 219 | 43.062 | 33.560 | 48.890 | 1.00 | 38.43 | A | O |
| ATOM | 1611 | C | SER | A | 219 | 40.212 | 33.591 | 48.899 | 1.00 | 38.30 | A | C |
| ATOM | 1612 | O | SER | A | 219 | 40.326 | 33.773 | 47.686 | 1.00 | 37.27 | A | O |
| ATOM | 1613 | N | PRO | A | 220 | 39.404 | 34.347 | 49.657 | 1.00 | 37.27 | A | N |
| ATOM | 1614 | CD | PRO | A | 220 | 39.059 | 34.239 | 51.088 | 1.00 | 34.74 | A | C |
| ATOM | 1615 | CA | PRO | A | 220 | 38.630 | 35.408 | 49.015 | 1.00 | 35.79 | A | C |
| ATOM | 1616 | CB | PRO | A | 220 | 37.560 | 35.718 | 50.054 | 1.00 | 35.96 | A | C |
| ATOM | 1617 | CG | PRO | A | 220 | 38.330 | 35.581 | 51.319 | 1.00 | 35.43 | A | C |
| ATOM | 1618 | C | PRO | A | 220 | 39.507 | 36.593 | 48.699 | 1.00 | 34.97 | A | C |
| ATOM | 1619 | O | PRO | A | 220 | 40.548 | 36.768 | 49.315 | 1.00 | 34.43 | A | O |
| ATOM | 1620 | N | ASP | A | 221 | 39.088 | 37.401 | 47.735 | 1.00 | 35.88 | A | N |
| ATOM | 1621 | CA | ASP | A | 221 | 39.863 | 38.566 | 47.348 | 1.00 | 36.52 | A | C |
| ATOM | 1622 | CB | ASP | A | 221 | 39.588 | 38.928 | 45.883 | 1.00 | 39.27 | A | C |
| ATOM | 1623 | CG | ASP | A | 221 | 40.059 | 37.848 | 44.927 | 1.00 | 40.28 | A | C |
| ATOM | 1624 | OD1 | ASP | A | 221 | 41.173 | 37.310 | 45.143 | 1.00 | 41.15 | A | O |
| ATOM | 1625 | OD2 | ASP | A | 221 | 39.320 | 37.545 | 43.968 | 1.00 | 42.19 | A | O |
| ATOM | 1626 | C | ASP | A | 221 | 39.554 | 39.741 | 48.248 | 1.00 | 36.16 | A | C |
| ATOM | 1627 | O | ASP | A | 221 | 38.731 | 39.630 | 49.155 | 1.00 | 35.13 | A | O |
| ATOM | 1628 | N | LEU | A | 222 | 40.217 | 40.862 | 47.995 | 1.00 | 34.55 | A | N |
| ATOM | 1629 | CA | LEU | A | 222 | 40.024 | 42.054 | 48.794 | 1.00 | 35.28 | A | C |
| ATOM | 1630 | CB | LEU | A | 222 | 41.323 | 42.423 | 49.523 | 1.00 | 34.77 | A | C |
| ATOM | 1631 | CG | LEU | A | 222 | 41.353 | 43.791 | 50.229 | 1.00 | 35.23 | A | C |
| ATOM | 1632 | CD1 | LEU | A | 222 | 40.567 | 43.718 | 51.535 | 1.00 | 32.84 | A | C |
| ATOM | 1633 | CD2 | LEU | A | 222 | 42.794 | 44.213 | 50.502 | 1.00 | 34.74 | A | C |
| ATOM | 1634 | C | LEU | A | 222 | 39.581 | 43.243 | 47.960 | 1.00 | 36.51 | A | C |
| ATOM | 1635 | O | LEU | A | 222 | 40.044 | 43.441 | 46.836 | 1.00 | 37.70 | A | O |
| ATOM | 1636 | N | GLU | A | 223 | 38.671 | 44.025 | 48.519 | 1.00 | 35.37 | A | N |
| ATOM | 1637 | CA | GLU | A | 223 | 38.197 | 45.228 | 47.873 | 1.00 | 34.46 | A | C |
| ATOM | 1638 | CB | GLU | A | 223 | 36.740 | 45.086 | 47.449 | 1.00 | 35.66 | A | C |
| ATOM | 1639 | CG | GLU | A | 223 | 36.549 | 44.496 | 46.065 | 1.00 | 39.74 | A | C |
| ATOM | 1640 | CD | GLU | A | 223 | 35.085 | 44.322 | 45.709 | 1.00 | 41.63 | A | C |
| ATOM | 1641 | OE1 | GLU | A | 223 | 34.309 | 45.288 | 45.881 | 1.00 | 43.04 | A | O |
| ATOM | 1642 | OE2 | GLU | A | 223 | 34.709 | 43.222 | 45.256 | 1.00 | 43.15 | A | O |
| ATOM | 1643 | C | GLU | A | 223 | 38.338 | 46.297 | 48.941 | 1.00 | 34.99 | A | C |
| ATOM | 1644 | O | GLU | A | 223 | 37.897 | 46.110 | 50.070 | 1.00 | 36.58 | A | O |
| ATOM | 1645 | N | ILE | A | 224 | 38.994 | 47.398 | 48.606 | 1.00 | 33.04 | A | N |
| ATOM | 1646 | CA | ILE | A | 224 | 39.157 | 48.470 | 49.560 | 1.00 | 32.69 | A | C |
| ATOM | 1647 | CB | ILE | A | 224 | 40.634 | 48.771 | 49.873 | 1.00 | 34.95 | A | C |
| ATOM | 1648 | CG2 | ILE | A | 224 | 40.721 | 49.903 | 50.908 | 1.00 | 32.94 | A | C |
| ATOM | 1649 | CG1 | ILE | A | 224 | 41.327 | 47.528 | 50.424 | 1.00 | 34.86 | A | C |
| ATOM | 1650 | CD1 | ILE | A | 224 | 42.798 | 47.747 | 50.716 | 1.00 | 37.35 | A | C |
| ATOM | 1651 | C | ILE | A | 224 | 38.566 | 49.719 | 48.977 | 1.00 | 32.76 | A | C |
| ATOM | 1652 | O | ILE | A | 224 | 38.756 | 50.017 | 47.807 | 1.00 | 35.48 | A | O |
| ATOM | 1653 | N | GLN | A | 225 | 37.852 | 50.454 | 49.810 | 1.00 | 33.65 | A | N |
| ATOM | 1654 | CA | GLN | A | 225 | 37.238 | 51.697 | 49.402 | 1.00 | 34.90 | A | C |
| ATOM | 1655 | CB | GLN | A | 225 | 35.749 | 51.486 | 49.112 | 1.00 | 37.51 | A | C |
| ATOM | 1656 | CG | GLN | A | 225 | 34.997 | 52.781 | 48.849 | 1.00 | 44.63 | A | C |
| ATOM | 1657 | CD | GLN | A | 225 | 33.489 | 52.605 | 48.843 | 1.00 | 49.85 | A | C |
| ATOM | 1658 | OE1 | GLN | A | 225 | 32.743 | 53.587 | 48.771 | 1.00 | 52.28 | A | O |
| ATOM | 1659 | NE2 | GLN | A | 225 | 33.030 | 51.354 | 48.914 | 1.00 | 50.33 | A | N |
| ATOM | 1660 | C | GLN | A | 225 | 37.387 | 52.669 | 50.560 | 1.00 | 34.06 | A | C |
| ATOM | 1661 | O | GLN | A | 225 | 37.145 | 52.307 | 51.711 | 1.00 | 34.26 | A | O |
| ATOM | 1662 | N | GLU | A | 226 | 37.807 | 53.893 | 50.281 | 1.00 | 33.61 | A | N |
| ATOM | 1663 | CA | GLU | A | 226 | 37.900 | 54.835 | 51.363 | 1.00 | 35.63 | A | C |
| ATOM | 1664 | CB | GLU | A | 226 | 39.360 | 55.170 | 51.689 | 1.00 | 38.42 | A | C |
| ATOM | 1665 | CG | GLU | A | 226 | 40.276 | 55.544 | 50.569 | 1.00 | 37.57 | A | C |
| ATOM | 1666 | CD | GLU | A | 226 | 41.692 | 55.767 | 51.090 | 1.00 | 38.82 | A | C |
| ATOM | 1667 | OE1 | GLU | A | 226 | 42.336 | 54.779 | 51.510 | 1.00 | 36.39 | A | O |
| ATOM | 1668 | OE2 | GLU | A | 226 | 42.152 | 56.932 | 51.099 | 1.00 | 38.05 | A | O |
| ATOM | 1669 | C | GLU | A | 226 | 37.064 | 56.086 | 51.137 | 1.00 | 35.77 | A | C |
| ATOM | 1670 | O | GLU | A | 226 | 36.803 | 56.488 | 50.001 | 1.00 | 34.87 | A | O |
| ATOM | 1671 | N | ILE | A | 227 | 36.609 | 56.665 | 52.242 | 1.00 | 35.20 | A | N |
| ATOM | 1672 | CA | ILE | A | 227 | 35.772 | 57.851 | 52.217 | 1.00 | 35.71 | A | C |
| ATOM | 1673 | CB | ILE | A | 227 | 34.386 | 57.574 | 52.878 | 1.00 | 38.12 | A | C |
| ATOM | 1674 | CG2 | ILE | A | 227 | 33.501 | 58.806 | 52.775 | 1.00 | 35.33 | A | C |
| ATOM | 1675 | CG1 | ILE | A | 227 | 33.713 | 56.355 | 52.235 | 1.00 | 40.43 | A | C |
| ATOM | 1676 | CD1 | ILE | A | 227 | 33.267 | 56.563 | 50.809 | 1.00 | 40.71 | A | C |
| ATOM | 1677 | C | ILE | A | 227 | 36.416 | 58.990 | 52.994 | 1.00 | 34.89 | A | C |
| ATOM | 1678 | O | ILE | A | 227 | 36.288 | 59.051 | 54.210 | 1.00 | 34.95 | A | O |
| ATOM | 1679 | N | ASN | A | 228 | 37.123 | 59.878 | 52.311 | 1.00 | 35.09 | A | N |
| ATOM | 1680 | CA | ASN | A | 228 | 37.706 | 61.023 | 52.995 | 1.00 | 36.11 | A | C |
| ATOM | 1681 | CB | ASN | A | 228 | 38.983 | 61.465 | 52.296 | 1.00 | 34.21 | A | C |
| ATOM | 1682 | CG | ASN | A | 228 | 39.800 | 62.407 | 53.132 | 1.00 | 33.22 | A | C |
| ATOM | 1683 | OD1 | ASN | A | 228 | 39.258 | 63.227 | 53.875 | 1.00 | 35.25 | A | O |
| ATOM | 1684 | ND2 | ASN | A | 228 | 41.121 | 62.313 | 53.008 | 1.00 | 32.88 | A | N |
| ATOM | 1685 | C | ASN | A | 228 | 36.625 | 62.100 | 52.863 | 1.00 | 37.68 | A | C |
| ATOM | 1686 | O | ASN | A | 228 | 36.531 | 62.777 | 51.844 | 1.00 | 38.45 | A | O |
| ATOM | 1687 | N | ALA | A | 229 | 35.805 | 62.235 | 53.898 | 1.00 | 39.34 | A | N |
| ATOM | 1688 | CA | ALA | A | 229 | 34.700 | 63.184 | 53.911 | 1.00 | 41.69 | A | C |
| ATOM | 1689 | CB | ALA | A | 229 | 33.998 | 63.112 | 55.252 | 1.00 | 40.50 | A | C |
| ATOM | 1690 | C | ALA | A | 229 | 35.044 | 64.637 | 53.581 | 1.00 | 42.87 | A | C |
| ATOM | 1691 | O | ALA | A | 229 | 34.388 | 65.252 | 52.737 | 1.00 | 42.66 | A | O |
| ATOM | 1692 | N | ALA | A | 230 | 36.059 | 65.180 | 54.250 | 1.00 | 44.27 | A | N |
| ATOM | 1693 | CA | ALA | A | 230 | 36.472 | 66.570 | 54.045 | 1.00 | 44.27 | A | C |
| ATOM | 1694 | CB | ALA | A | 230 | 37.181 | 67.092 | 55.291 | 1.00 | 42.61 | A | C |
| ATOM | 1695 | C | ALA | A | 230 | 37.354 | 66.780 | 52.818 | 1.00 | 45.76 | A | C |
| ATOM | 1696 | O | ALA | A | 230 | 37.377 | 67.875 | 52.255 | 1.00 | 46.63 | A | O |
| ATOM | 1697 | N | ASN | A | 231 | 38.081 | 65.742 | 52.408 | 1.00 | 46.35 | A | N |
| ATOM | 1698 | CA | ASN | A | 231 | 38.946 | 65.837 | 51.235 | 1.00 | 46.36 | A | C |
| ATOM | 1699 | CB | ASN | A | 231 | 40.393 | 66.047 | 51.665 | 1.00 | 46.15 | A | C |
| ATOM | 1700 | CG | ASN | A | 231 | 40.589 | 67.375 | 52.366 | 1.00 | 47.62 | A | C |
| ATOM | 1701 | OD1 | ASN | A | 231 | 40.164 | 67.556 | 53.506 | 1.00 | 46.95 | A | O |
| ATOM | 1702 | ND2 | ASN | A | 231 | 41.216 | 68.321 | 51.679 | 1.00 | 49.85 | A | N |
| ATOM | 1703 | C | ASN | A | 231 | 38.809 | 64.614 | 50.334 | 1.00 | 47.54 | A | C |
| ATOM | 1704 | O | ASN | A | 231 | 39.679 | 63.743 | 50.272 | 1.00 | 45.76 | A | O |
| ATOM | 1705 | N | SER | A | 232 | 37.689 | 64.602 | 49.620 | 1.00 | 49.17 | A | N |
| ATOM | 1706 | CA | SER | A | 232 | 37.278 | 63.545 | 48.707 | 1.00 | 52.25 | A | C |
| ATOM | 1707 | CB | SER | A | 232 | 36.544 | 64.161 | 47.513 | 1.00 | 53.58 | A | C |
| ATOM | 1708 | OG | SER | A | 232 | 35.848 | 63.164 | 46.777 | 1.00 | 52.85 | A | O |
| ATOM | 1709 | C | SER | A | 232 | 38.294 | 62.519 | 48.189 | 1.00 | 54.50 | A | C |
| ATOM | 1710 | O | SER | A | 232 | 38.194 | 61.342 | 48.521 | 1.00 | 56.60 | A | O |
| ATOM | 1711 | N | LYS | A | 233 | 39.255 | 62.922 | 47.365 | 1.00 | 55.77 | A | N |
| ATOM | 1712 | CA | LYS | A | 233 | 40.193 | 61.926 | 46.841 | 1.00 | 57.12 | A | C |
| ATOM | 1713 | CB | LYS | A | 233 | 40.287 | 62.028 | 45.311 | 1.00 | 60.73 | A | C |
| ATOM | 1714 | CG | LYS | A | 233 | 39.433 | 61.018 | 44.526 | 1.00 | 63.59 | A | C |
| ATOM | 1715 | CD | LYS | A | 233 | 37.947 | 61.319 | 44.620 | 1.00 | 67.22 | A | C |
| ATOM | 1716 | CE | LYS | A | 233 | 37.129 | 60.417 | 43.694 | 1.00 | 69.63 | A | C |
| ATOM | 1717 | NZ | LYS | A | 233 | 37.410 | 60.654 | 42.242 | 1.00 | 72.45 | A | N |
| ATOM | 1718 | C | LYS | A | 233 | 41.601 | 61.935 | 47.432 | 1.00 | 56.29 | A | C |
| ATOM | 1719 | O | LYS | A | 233 | 42.529 | 61.359 | 46.854 | 1.00 | 56.44 | A | O |
| ATOM | 1720 | N | GLN | A | 234 | 41.775 | 62.574 | 48.581 | 1.00 | 53.62 | A | N |
| ATOM | 1721 | CA | GLN | A | 234 | 43.095 | 62.593 | 49.192 | 1.00 | 52.15 | A | C |
| ATOM | 1722 | CB | GLN | A | 234 | 43.258 | 63.842 | 50.062 | 1.00 | 54.75 | A | C |
| ATOM | 1723 | CG | GLN | A | 234 | 43.266 | 65.166 | 49.286 | 1.00 | 61.06 | A | C |
| ATOM | 1724 | CD | GLN | A | 234 | 44.357 | 65.228 | 48.217 | 1.00 | 64.05 | A | C |
| ATOM | 1725 | OE1 | GLN | A | 234 | 44.250 | 64.592 | 47.164 | 1.00 | 65.23 | A | O |
| ATOM | 1726 | NE2 | GLN | A | 234 | 45.415 | 65.991 | 48.489 | 1.00 | 65.37 | A | N |
| ATOM | 1727 | C | GLN | A | 234 | 43.279 | 61.326 | 50.028 | 1.00 | 48.19 | A | C |
| ATOM | 1728 | O | GLN | A | 234 | 42.395 | 60.949 | 50.791 | 1.00 | 46.68 | A | O |
| ATOM | 1729 | N | PRO | A | 235 | 44.425 | 60.639 | 49.879 | 1.00 | 46.51 | A | N |
| ATOM | 1730 | CD | PRO | A | 235 | 45.547 | 60.903 | 48.956 | 1.00 | 45.42 | A | C |
| ATOM | 1731 | CA | PRO | A | 235 | 44.661 | 59.414 | 50.657 | 1.00 | 44.41 | A | C |
| ATOM | 1732 | CB | PRO | A | 235 | 46.092 | 59.034 | 50.282 | 1.00 | 44.00 | A | C |
| ATOM | 1733 | CG | PRO | A | 235 | 46.213 | 59.546 | 48.868 | 1.00 | 44.56 | A | C |
| ATOM | 1734 | C | PRO | A | 235 | 44.497 | 59.672 | 52.159 | 1.00 | 41.88 | A | C |
| ATOM | 1735 | O | PRO | A | 235 | 44.904 | 60.720 | 52.661 | 1.00 | 40.85 | A | O |
| ATOM | 1736 | N | ILE | A | 236 | 43.894 | 58.713 | 52.861 | 1.00 | 38.29 | A | N |
| ATOM | 1737 | CA | ILE | A | 236 | 43.658 | 58.825 | 54.295 | 1.00 | 36.08 | A | C |
| ATOM | 1738 | CB | ILE | A | 236 | 42.451 | 57.953 | 54.716 | 1.00 | 33.99 | A | C |
| ATOM | 1739 | CG2 | ILE | A | 236 | 42.328 | 57.915 | 56.231 | 1.00 | 30.34 | A | C |
| ATOM | 1740 | CG1 | ILE | A | 236 | 41.175 | 58.521 | 54.083 | 1.00 | 32.74 | A | C |
| ATOM | 1741 | CD1 | ILE | A | 236 | 39.938 | 57.657 | 54.252 | 1.00 | 31.92 | A | C |
| ATOM | 1742 | C | ILE | A | 236 | 44.882 | 58.423 | 55.108 | 1.00 | 34.45 | A | C |
| ATOM | 1743 | O | ILE | A | 236 | 45.352 | 57.293 | 55.007 | 1.00 | 33.54 | A | O |
| ATOM | 1744 | N | HIS | A | 237 | 45.393 | 59.366 | 55.901 | 1.00 | 34.75 | A | N |
| ATOM | 1745 | CA | HIS | A | 237 | 46.565 | 59.145 | 56.755 | 1.00 | 34.97 | A | C |
| ATOM | 1746 | CB | HIS | A | 237 | 47.759 | 60.002 | 56.304 | 1.00 | 36.89 | A | C |
| ATOM | 1747 | CG | HIS | A | 237 | 48.298 | 59.644 | 54.951 | 1.00 | 46.02 | A | C |
| ATOM | 1748 | CD2 | HIS | A | 237 | 49.366 | 58.892 | 54.587 | 1.00 | 47.39 | A | C |
| ATOM | 1749 | ND1 | HIS | A | 237 | 47.716 | 60.072 | 53.775 | 1.00 | 49.74 | A | N |
| ATOM | 1750 | CE1 | HIS | A | 237 | 48.401 | 59.599 | 52.748 | 1.00 | 48.94 | A | C |
| ATOM | 1751 | NE2 | HIS | A | 237 | 49.406 | 58.879 | 53.213 | 1.00 | 48.71 | A | N |
| ATOM | 1752 | C | HIS | A | 237 | 46.210 | 59.535 | 58.187 | 1.00 | 33.22 | A | C |
| ATOM | 1753 | O | HIS | A | 237 | 45.265 | 60.293 | 58.418 | 1.00 | 32.40 | A | O |
| ATOM | 1754 | N | MET | A | 238 | 46.978 | 59.030 | 59.145 | 1.00 | 30.67 | A | N |
| ATOM | 1755 | CA | MET | A | 238 | 46.737 | 59.341 | 60.546 | 1.00 | 29.27 | A | C |
| ATOM | 1756 | CB | MET | A | 238 | 45.613 | 58.456 | 61.102 | 1.00 | 28.09 | A | C |
| ATOM | 1757 | CG | MET | A | 238 | 46.018 | 56.992 | 61.311 | 1.00 | 27.72 | A | C |
| ATOM | 1758 | SD | MET | A | 238 | 44.766 | 55.984 | 62.161 | 1.00 | 28.44 | A | S |
| ATOM | 1759 | CE | MET | A | 238 | 44.560 | 56.919 | 63.668 | 1.00 | 21.65 | A | C |
| ATOM | 1760 | C | MET | A | 238 | 48.007 | 59.096 | 61.356 | 1.00 | 30.28 | A | C |
| ATOM | 1761 | O | MET | A | 238 | 48.947 | 58.450 | 60.882 | 1.00 | 30.46 | A | O |
| ATOM | 1762 | N | VAL | A | 239 | 48.030 | 59.631 | 62.573 | 1.00 | 29.34 | A | N |
| ATOM | 1763 | CA | VAL | A | 239 | 49.150 | 59.438 | 63.484 | 1.00 | 27.34 | A | C |
| ATOM | 1764 | CB | VAL | A | 239 | 49.331 | 60.666 | 64.422 | 1.00 | 28.70 | A | C |
| ATOM | 1765 | CG1 | VAL | A | 239 | 50.494 | 60.429 | 65.385 | 1.00 | 22.44 | A | C |
| ATOM | 1766 | CG2 | VAL | A | 239 | 49.569 | 61.931 | 63.596 | 1.00 | 25.02 | A | C |
| ATOM | 1767 | C | VAL | A | 239 | 48.732 | 58.215 | 64.313 | 1.00 | 29.57 | A | C |
| ATOM | 1768 | O | VAL | A | 239 | 47.734 | 58.270 | 65.041 | 1.00 | 29.45 | A | O |
| ATOM | 1769 | N | TYR | A | 240 | 49.468 | 57.111 | 64.203 | 1.00 | 26.67 | A | N |
| ATOM | 1770 | CA | TYR | A | 240 | 49.089 | 55.909 | 64.942 | 1.00 | 26.64 | A | C |
| ATOM | 1771 | CB | TYR | A | 240 | 47.906 | 55.234 | 64.235 | 1.00 | 27.23 | A | C |
| ATOM | 1772 | CG | TYR | A | 240 | 47.065 | 54.353 | 65.143 | 1.00 | 27.39 | A | C |
| ATOM | 1773 | CD1 | TYR | A | 240 | 47.046 | 52.957 | 64.995 | 1.00 | 25.89 | A | C |
| ATOM | 1774 | CE1 | TYR | A | 240 | 46.317 | 52.142 | 65.885 | 1.00 | 23.64 | A | C |
| ATOM | 1775 | CD2 | TYR | A | 240 | 46.330 | 54.915 | 66.191 | 1.00 | 23.34 | A | C |
| ATOM | 1776 | CE2 | TYR | A | 240 | 45.603 | 54.118 | 67.074 | 1.00 | 23.49 | A | C |
| ATOM | 1777 | CZ | TYR | A | 240 | 45.604 | 52.736 | 66.923 | 1.00 | 24.28 | A | C |
| ATOM | 1778 | OH | TYR | A | 240 | 44.931 | 51.966 | 67.836 | 1.00 | 22.57 | A | O |
| ATOM | 1779 | C | TYR | A | 240 | 50.236 | 54.914 | 65.080 | 1.00 | 27.17 | A | C |
| ATOM | 1780 | O | TYR | A | 240 | 51.314 | 55.112 | 64.519 | 1.00 | 28.26 | A | O |
| ATOM | 1781 | N | VAL | A | 241 | 50.004 | 53.851 | 65.846 | 1.00 | 27.16 | A | N |
| ATOM | 1782 | CA | VAL | A | 241 | 51.013 | 52.816 | 66.041 | 1.00 | 26.26 | A | C |
| ATOM | 1783 | CB | VAL | A | 241 | 50.962 | 52.246 | 67.478 | 1.00 | 25.52 | A | C |
| ATOM | 1784 | CG1 | VAL | A | 241 | 52.129 | 51.287 | 67.699 | 1.00 | 23.83 | A | C |
| ATOM | 1785 | CG2 | VAL | A | 241 | 51.019 | 53.388 | 68.500 | 1.00 | 24.07 | A | C |
| ATOM | 1786 | C | VAL | A | 241 | 50.617 | 51.742 | 65.041 | 1.00 | 25.70 | A | C |
| ATOM | 1787 | O | VAL | A | 241 | 49.754 | 50.916 | 65.326 | 1.00 | 24.95 | A | O |
| ATOM | 1788 | N | PRO | A | 242 | 51.239 | 51.748 | 63.847 | 1.00 | 26.79 | A | N |
| ATOM | 1789 | CD | PRO | A | 242 | 52.317 | 52.644 | 63.390 | 1.00 | 24.44 | A | C |
| ATOM | 1790 | CA | PRO | A | 242 | 50.909 | 50.757 | 62.814 | 1.00 | 26.73 | A | C |
| ATOM | 1791 | CB | PRO | A | 242 | 51.908 | 51.071 | 61.692 | 1.00 | 25.55 | A | C |
| ATOM | 1792 | CG | PRO | A | 242 | 53.024 | 51.786 | 62.392 | 1.00 | 26.95 | A | C |
| ATOM | 1793 | C | PRO | A | 242 | 50.874 | 49.279 | 63.211 | 1.00 | 26.85 | A | C |
| ATOM | 1794 | O | PRO | A | 242 | 50.007 | 48.534 | 62.741 | 1.00 | 24.77 | A | O |
| ATOM | 1795 | N | SER | A | 243 | 51.785 | 48.845 | 64.078 | 1.00 | 26.42 | A | N |
| ATOM | 1796 | CA | SER | A | 243 | 51.771 | 47.442 | 64.476 | 1.00 | 28.46 | A | C |
| ATOM | 1797 | CB | SER | A | 243 | 53.015 | 47.075 | 65.300 | 1.00 | 27.54 | A | C |
| ATOM | 1798 | OG | SER | A | 243 | 53.102 | 47.835 | 66.476 | 1.00 | 34.10 | A | O |
| ATOM | 1799 | C | SER | A | 243 | 50.499 | 47.115 | 65.244 | 1.00 | 26.73 | A | C |
| ATOM | 1800 | O | SER | A | 243 | 49.988 | 45.997 | 65.152 | 1.00 | 27.41 | A | O |
| ATOM | 1801 | N | HIS | A | 244 | 49.987 | 48.075 | 66.010 | 1.00 | 25.54 | A | N |
| ATOM | 1802 | CA | HIS | A | 244 | 48.738 | 47.846 | 66.741 | 1.00 | 26.32 | A | C |
| ATOM | 1803 | CB | HIS | A | 244 | 48.403 | 49.040 | 67.634 | 1.00 | 23.67 | A | C |
| ATOM | 1804 | CG | HIS | A | 244 | 49.205 | 49.115 | 68.893 | 1.00 | 26.55 | A | C |
| ATOM | 1805 | CD2 | HIS | A | 244 | 49.023 | 49.851 | 70.017 | 1.00 | 24.37 | A | C |
| ATOM | 1806 | ND1 | HIS | A | 244 | 50.386 | 48.426 | 69.072 | 1.00 | 27.63 | A | N |
| ATOM | 1807 | CE1 | HIS | A | 244 | 50.899 | 48.737 | 70.251 | 1.00 | 27.20 | A | C |
| ATOM | 1808 | NE2 | HIS | A | 244 | 50.092 | 49.601 | 70.842 | 1.00 | 27.05 | A | N |
| ATOM | 1809 | C | HIS | A | 244 | 47.590 | 47.658 | 65.727 | 1.00 | 27.14 | A | C |
| ATOM | 1810 | O | HIS | A | 244 | 46.734 | 46.777 | 65.879 | 1.00 | 26.10 | A | O |
| ATOM | 1811 | N | LEU | A | 245 | 47.582 | 48.502 | 64.698 | 1.00 | 26.41 | A | N |
| ATOM | 1812 | CA | LEU | A | 245 | 46.556 | 48.450 | 63.666 | 1.00 | 28.60 | A | C |
| ATOM | 1813 | CB | LEU | A | 245 | 46.703 | 49.642 | 62.699 | 1.00 | 28.03 | A | C |
| ATOM | 1814 | CG | LEU | A | 245 | 45.665 | 49.716 | 61.567 | 1.00 | 28.96 | A | C |
| ATOM | 1815 | CD1 | LEU | A | 245 | 44.262 | 49.658 | 62.158 | 1.00 | 23.62 | A | C |
| ATOM | 1816 | CD2 | LEU | A | 245 | 45.856 | 50.989 | 60.753 | 1.00 | 22.60 | A | C |
| ATOM | 1817 | C | LEU | A | 245 | 46.645 | 47.139 | 62.897 | 1.00 | 28.37 | A | C |
| ATOM | 1818 | O | LEU | A | 245 | 45.624 | 46.530 | 62.573 | 1.00 | 28.37 | A | O |
| ATOM | 1819 | N | TYR | A | 246 | 47.874 | 46.715 | 62.612 | 1.00 | 28.32 | A | N |
| ATOM | 1820 | CA | TYR | A | 246 | 48.132 | 45.467 | 61.895 | 1.00 | 26.37 | A | C |
| ATOM | 1821 | CB | TYR | A | 246 | 49.639 | 45.271 | 61.710 | 1.00 | 25.13 | A | C |
| ATOM | 1822 | CG | TYR | A | 246 | 49.996 | 43.995 | 60.986 | 1.00 | 26.69 | A | C |
| ATOM | 1823 | CD1 | TYR | A | 246 | 50.246 | 43.996 | 59.613 | 1.00 | 25.83 | A | C |
| ATOM | 1824 | CE1 | TYR | A | 246 | 50.534 | 42.817 | 58.934 | 1.00 | 26.00 | A | C |
| ATOM | 1825 | CD2 | TYR | A | 246 | 50.046 | 42.775 | 61.664 | 1.00 | 23.18 | A | C |
| ATOM | 1826 | CE2 | TYR | A | 246 | 50.334 | 41.585 | 60.993 | 1.00 | 23.35 | A | C |
| ATOM | 1827 | CZ | TYR | A | 246 | 50.575 | 41.612 | 59.629 | 1.00 | 27.36 | A | C |
| ATOM | 1828 | OH | TYR | A | 246 | 50.837 | 40.434 | 58.952 | 1.00 | 26.29 | A | O |
| ATOM | 1829 | C | TYR | A | 246 | 47.564 | 44.287 | 62.693 | 1.00 | 28.01 | A | C |
| ATOM | 1830 | O | TYR | A | 246 | 46.828 | 43.453 | 62.162 | 1.00 | 26.35 | A | O |
| ATOM | 1831 | N | HIS | A | 247 | 47.907 | 44.229 | 63.975 | 1.00 | 27.90 | A | N |
| ATOM | 1832 | CA | HIS | A | 247 | 47.434 | 43.157 | 64.833 | 1.00 | 29.69 | A | C |
| ATOM | 1833 | CB | HIS | A | 247 | 47.860 | 43.413 | 66.289 | 1.00 | 33.29 | A | C |
| ATOM | 1834 | CG | HIS | A | 247 | 47.509 | 42.301 | 67.235 | 1.00 | 41.39 | A | C |
| ATOM | 1835 | CD2 | HIS | A | 247 | 46.325 | 41.702 | 67.515 | 1.00 | 43.62 | A | C |
| ATOM | 1836 | ND1 | HIS | A | 247 | 48.448 | 41.679 | 68.034 | 1.00 | 45.43 | A | N |
| ATOM | 1837 | CE1 | HIS | A | 247 | 47.858 | 40.744 | 68.760 | 1.00 | 44.71 | A | C |
| ATOM | 1838 | NE2 | HIS | A | 247 | 46.570 | 40.737 | 68.465 | 1.00 | 44.69 | A | N |
| ATOM | 1839 | C | HIS | A | 247 | 45.919 | 43.010 | 64.745 | 1.00 | 28.67 | A | C |
| ATOM | 1840 | O | HIS | A | 247 | 45.413 | 41.909 | 64.520 | 1.00 | 27.21 | A | O |
| ATOM | 1841 | N | MET | A | 248 | 45.192 | 44.115 | 64.905 | 1.00 | 28.44 | A | N |
| ATOM | 1842 | CA | MET | A | 248 | 43.732 | 44.061 | 64.858 | 1.00 | 27.18 | A | C |
| ATOM | 1843 | CB | MET | A | 248 | 43.127 | 45.431 | 65.181 | 1.00 | 24.15 | A | C |
| ATOM | 1844 | CG | MET | A | 248 | 43.541 | 45.988 | 66.534 | 1.00 | 24.77 | A | C |
| ATOM | 1845 | SD | MET | A | 248 | 42.710 | 47.557 | 66.943 | 1.00 | 28.00 | A | S |
| ATOM | 1846 | CE | MET | A | 248 | 43.648 | 48.704 | 66.013 | 1.00 | 16.76 | A | C |
| ATOM | 1847 | C | MET | A | 248 | 43.224 | 43.572 | 63.502 | 1.00 | 26.36 | A | C |
| ATOM | 1848 | O | MET | A | 248 | 42.449 | 42.609 | 63.434 | 1.00 | 26.57 | A | O |
| ATOM | 1849 | N | LEU | A | 249 | 43.667 | 44.226 | 62.429 | 1.00 | 25.98 | A | N |
| ATOM | 1850 | CA | LEU | A | 249 | 43.255 | 43.857 | 61.074 | 1.00 | 26.65 | A | C |
| ATOM | 1851 | CB | LEU | A | 249 | 43.885 | 44.801 | 60.042 | 1.00 | 23.66 | A | C |
| ATOM | 1852 | CG | LEU | A | 249 | 43.270 | 46.207 | 60.030 | 1.00 | 24.80 | A | C |
| ATOM | 1853 | CD1 | LEU | A | 249 | 44.158 | 47.185 | 59.269 | 1.00 | 22.73 | A | C |
| ATOM | 1854 | CD2 | LEU | A | 249 | 41.880 | 46.128 | 59.402 | 1.00 | 20.61 | A | C |
| ATOM | 1855 | C | LEU | A | 249 | 43.593 | 42.413 | 60.734 | 1.00 | 25.33 | A | C |
| ATOM | 1856 | O | LEU | A | 249 | 42.786 | 41.719 | 60.127 | 1.00 | 28.29 | A | O |
| ATOM | 1857 | N | PHE | A | 250 | 44.775 | 41.956 | 61.132 | 1.00 | 26.01 | A | N |
| ATOM | 1858 | CA | PHE | A | 250 | 45.179 | 40.577 | 60.865 | 1.00 | 27.40 | A | C |
| ATOM | 1859 | CB | PHE | A | 250 | 46.607 | 40.328 | 61.382 | 1.00 | 24.98 | A | C |
| ATOM | 1860 | CG | PHE | A | 250 | 47.131 | 38.956 | 61.072 | 1.00 | 25.65 | A | C |
| ATOM | 1861 | CD1 | PHE | A | 250 | 47.223 | 38.513 | 59.752 | 1.00 | 24.95 | A | C |
| ATOM | 1862 | CD2 | PHE | A | 250 | 47.523 | 38.100 | 62.098 | 1.00 | 28.05 | A | C |
| ATOM | 1863 | CE1 | PHE | A | 250 | 47.698 | 37.231 | 59.454 | 1.00 | 25.09 | A | C |
| ATOM | 1864 | CE2 | PHE | A | 250 | 48.002 | 36.818 | 61.815 | 1.00 | 28.36 | A | C |
| ATOM | 1865 | CZ | PHE | A | 250 | 48.088 | 36.383 | 60.485 | 1.00 | 25.97 | A | C |
| ATOM | 1866 | C | PHE | A | 250 | 44.202 | 39.594 | 61.531 | 1.00 | 25.82 | A | C |
| ATOM | 1867 | O | PHE | A | 250 | 43.796 | 38.596 | 60.933 | 1.00 | 26.67 | A | O |
| ATOM | 1868 | N | GLU | A | 251 | 43.837 | 39.882 | 62.774 | 1.00 | 26.55 | A | N |
| ATOM | 1869 | CA | GLU | A | 251 | 42.907 | 39.033 | 63.509 | 1.00 | 29.24 | A | C |
| ATOM | 1870 | CB | GLU | A | 251 | 42.711 | 39.558 | 64.935 | 1.00 | 32.08 | A | C |
| ATOM | 1871 | CG | GLU | A | 251 | 41.623 | 38.807 | 65.700 | 1.00 | 40.51 | A | C |
| ATOM | 1872 | CD | GLU | A | 251 | 41.963 | 37.328 | 65.895 | 1.00 | 48.07 | A | C |
| ATOM | 1873 | OE1 | GLU | A | 251 | 41.032 | 36.532 | 66.181 | 1.00 | 49.30 | A | O |
| ATOM | 1874 | OE2 | GLU | A | 251 | 43.164 | 36.972 | 65.770 | 1.00 | 47.76 | A | O |
| ATOM | 1875 | C | GLU | A | 251 | 41.552 | 38.982 | 62.805 | 1.00 | 28.82 | A | C |
| ATOM | 1876 | O | GLU | A | 251 | 40.959 | 37.910 | 62.661 | 1.00 | 29.30 | A | O |
| ATOM | 1877 | N | LEU | A | 252 | 41.073 | 40.143 | 62.360 | 1.00 | 25.79 | A | N |
| ATOM | 1878 | CA | LEU | A | 252 | 39.790 | 40.213 | 61.690 | 1.00 | 25.68 | A | C |
| ATOM | 1879 | CB | LEU | A | 252 | 39.330 | 41.668 | 61.589 | 1.00 | 24.34 | A | C |
| ATOM | 1880 | CG | LEU | A | 252 | 39.128 | 42.314 | 62.961 | 1.00 | 23.10 | A | C |
| ATOM | 1881 | CD1 | LEU | A | 252 | 38.708 | 43.742 | 62.785 | 1.00 | 20.09 | A | C |
| ATOM | 1882 | CD2 | LEU | A | 252 | 38.081 | 41.559 | 63.749 | 1.00 | 22.02 | A | C |
| ATOM | 1883 | C | LEU | A | 252 | 39.810 | 39.557 | 60.316 | 1.00 | 25.37 | A | C |
| ATOM | 1884 | O | LEU | A | 252 | 38.829 | 38.916 | 59.930 | 1.00 | 27.57 | A | O |
| ATOM | 1885 | N | PHE | A | 253 | 40.913 | 39.704 | 59.581 | 1.00 | 23.19 | A | N |
| ATOM | 1886 | CA | PHE | A | 253 | 40.996 | 39.084 | 58.264 | 1.00 | 24.64 | A | C |
| ATOM | 1887 | CB | PHE | A | 253 | 42.242 | 39.547 | 57.500 | 1.00 | 22.41 | A | C |
| ATOM | 1888 | CG | PHE | A | 253 | 42.047 | 40.815 | 56.709 | 1.00 | 20.83 | A | C |
| ATOM | 1889 | CD1 | PHE | A | 253 | 41.053 | 40.902 | 55.729 | 1.00 | 18.74 | A | C |
| ATOM | 1890 | CD2 | PHE | A | 253 | 42.895 | 41.906 | 56.905 | 1.00 | 19.15 | A | C |
| ATOM | 1891 | CE1 | PHE | A | 253 | 40.906 | 42.061 | 54.945 | 1.00 | 18.78 | A | C |
| ATOM | 1892 | CE2 | PHE | A | 253 | 42.764 | 43.073 | 56.134 | 1.00 | 18.22 | A | C |
| ATOM | 1893 | CZ | PHE | A | 253 | 41.768 | 43.150 | 55.149 | 1.00 | 18.16 | A | C |
| ATOM | 1894 | C | PHE | A | 253 | 41.011 | 37.562 | 58.395 | 1.00 | 24.61 | A | C |
| ATOM | 1895 | O | PHE | A | 253 | 40.448 | 36.863 | 57.561 | 1.00 | 27.16 | A | O |
| ATOM | 1896 | N | LYS | A | 254 | 41.643 | 37.048 | 59.445 | 1.00 | 26.39 | A | N |
| ATOM | 1897 | CA | LYS | A | 254 | 41.696 | 35.606 | 59.641 | 1.00 | 29.14 | A | C |
| ATOM | 1898 | CB | LYS | A | 254 | 42.590 | 35.255 | 60.835 | 1.00 | 29.43 | A | C |
| ATOM | 1899 | CG | LYS | A | 254 | 44.042 | 34.981 | 60.447 | 1.00 | 33.45 | A | C |
| ATOM | 1900 | CD | LYS | A | 254 | 45.032 | 35.395 | 61.539 | 1.00 | 35.33 | A | C |
| ATOM | 1901 | CE | LYS | A | 254 | 44.881 | 34.569 | 62.807 | 1.00 | 36.51 | A | C |
| ATOM | 1902 | NZ | LYS | A | 254 | 45.835 | 35.018 | 63.864 | 1.00 | 36.63 | A | N |
| ATOM | 1903 | C | LYS | A | 254 | 40.319 | 35.011 | 59.844 | 1.00 | 28.49 | A | C |
| ATOM | 1904 | O | LYS | A | 254 | 39.995 | 33.981 | 59.259 | 1.00 | 30.40 | A | O |
| ATOM | 1905 | N | ASN | A | 255 | 39.504 | 35.662 | 60.669 | 1.00 | 28.77 | A | N |
| ATOM | 1906 | CA | ASN | A | 255 | 38.159 | 35.169 | 60.938 | 1.00 | 28.98 | A | C |
| ATOM | 1907 | CB | ASN | A | 255 | 37.526 | 35.928 | 62.112 | 1.00 | 29.11 | A | C |
| ATOM | 1908 | CG | ASN | A | 255 | 38.353 | 35.823 | 63.387 | 1.00 | 31.06 | A | C |
| ATOM | 1909 | OD1 | ASN | A | 255 | 39.031 | 34.825 | 63.615 | 1.00 | 34.03 | A | O |
| ATOM | 1910 | ND2 | ASN | A | 255 | 38.286 | 36.848 | 64.229 | 1.00 | 30.22 | A | N |
| ATOM | 1911 | C | ASN | A | 255 | 37.280 | 35.296 | 59.702 | 1.00 | 27.45 | A | C |
| ATOM | 1912 | O | ASN | A | 255 | 36.556 | 34.361 | 59.360 | 1.00 | 27.50 | A | O |
| ATOM | 1913 | N | ALA | A | 256 | 37.363 | 36.442 | 59.030 | 1.00 | 26.48 | A | N |
| ATOM | 1914 | CA | ALA | A | 256 | 36.561 | 36.697 | 57.841 | 1.00 | 30.13 | A | C |
| ATOM | 1915 | CB | ALA | A | 256 | 36.782 | 38.142 | 57.356 | 1.00 | 27.96 | A | C |
| ATOM | 1916 | C | ALA | A | 256 | 36.881 | 35.703 | 56.722 | 1.00 | 32.34 | A | C |
| ATOM | 1917 | O | ALA | A | 256 | 35.986 | 35.265 | 55.990 | 1.00 | 33.70 | A | O |
| ATOM | 1918 | N | MET | A | 257 | 38.152 | 35.335 | 56.595 | 1.00 | 32.84 | A | N |
| ATOM | 1919 | CA | MET | A | 257 | 38.546 | 34.396 | 55.551 | 1.00 | 33.43 | A | C |
| ATOM | 1920 | CB | MET | A | 257 | 40.062 | 34.392 | 55.370 | 1.00 | 32.44 | A | C |
| ATOM | 1921 | CG | MET | A | 257 | 40.601 | 35.713 | 54.869 | 1.00 | 34.25 | A | C |
| ATOM | 1922 | SD | MET | A | 257 | 42.375 | 35.674 | 54.595 | 1.00 | 36.40 | A | S |
| ATOM | 1923 | CE | MET | A | 257 | 42.569 | 37.131 | 53.638 | 1.00 | 30.10 | A | C |
| ATOM | 1924 | C | MET | A | 257 | 38.062 | 33.000 | 55.871 | 1.00 | 31.54 | A | C |
| ATOM | 1925 | O | MET | A | 257 | 37.449 | 32.352 | 55.037 | 1.00 | 32.31 | A | O |
| ATOM | 1926 | N | ARG | A | 258 | 38.328 | 32.549 | 57.087 | 1.00 | 30.36 | A | N |
| ATOM | 1927 | CA | ARG | A | 258 | 37.910 | 31.225 | 57.514 | 1.00 | 31.82 | A | C |
| ATOM | 1928 | CB | ARG | A | 258 | 38.301 | 31.021 | 58.977 | 1.00 | 33.21 | A | C |
| ATOM | 1929 | CG | ARG | A | 258 | 37.653 | 29.843 | 59.685 | 1.00 | 42.01 | A | C |
| ATOM | 1930 | CD | ARG | A | 258 | 38.186 | 29.765 | 61.121 | 1.00 | 47.68 | A | C |
| ATOM | 1931 | NE | ARG | A | 258 | 37.267 | 29.126 | 62.067 | 1.00 | 56.17 | A | N |
| ATOM | 1932 | CZ | ARG | A | 258 | 37.057 | 27.813 | 62.166 | 1.00 | 61.18 | A | C |
| ATOM | 1933 | NH1 | ARG | A | 258 | 37.703 | 26.961 | 61.372 | 1.00 | 62.69 | A | N |
| ATOM | 1934 | NH2 | ARG | A | 258 | 36.191 | 27.347 | 63.061 | 1.00 | 61.40 | A | N |
| ATOM | 1935 | C | ARG | A | 258 | 36.404 | 31.065 | 57.315 | 1.00 | 30.64 | A | C |
| ATOM | 1936 | O | ARG | A | 258 | 35.960 | 30.128 | 56.654 | 1.00 | 29.69 | A | O |
| ATOM | 1937 | N | ALA | A | 259 | 35.623 | 31.997 | 57.860 | 1.00 | 29.89 | A | N |
| ATOM | 1938 | CA | ALA | A | 259 | 34.165 | 31.946 | 57.740 | 1.00 | 29.23 | A | C |
| ATOM | 1939 | CB | ALA | A | 259 | 33.540 | 33.138 | 58.471 | 1.00 | 24.80 | A | C |
| ATOM | 1940 | C | ALA | A | 259 | 33.712 | 31.928 | 56.274 | 1.00 | 28.28 | A | C |
| ATOM | 1941 | O | ALA | A | 259 | 32.814 | 31.169 | 55.895 | 1.00 | 28.50 | A | O |
| ATOM | 1942 | N | THR | A | 260 | 34.338 | 32.761 | 55.453 | 1.00 | 27.86 | A | N |
| ATOM | 1943 | CA | THR | A | 260 | 33.976 | 32.828 | 54.051 | 1.00 | 31.52 | A | C |
| ATOM | 1944 | CB | THR | A | 260 | 34.724 | 33.963 | 53.341 | 1.00 | 31.42 | A | C |
| ATOM | 1945 | OG1 | THR | A | 260 | 34.324 | 35.223 | 53.900 | 1.00 | 30.81 | A | O |
| ATOM | 1946 | CG2 | THR | A | 260 | 34.388 | 33.968 | 51.867 | 1.00 | 30.76 | A | C |
| ATOM | 1947 | C | THR | A | 260 | 34.264 | 31.514 | 53.341 | 1.00 | 34.19 | A | C |
| ATOM | 1948 | O | THR | A | 260 | 33.417 | 30.979 | 52.623 | 1.00 | 35.66 | A | O |
| ATOM | 1949 | N | VAL | A | 261 | 35.460 | 30.987 | 53.558 | 1.00 | 36.23 | A | N |
| ATOM | 1950 | CA | VAL | A | 261 | 35.855 | 29.738 | 52.938 | 1.00 | 36.98 | A | C |
| ATOM | 1951 | CB | VAL | A | 261 | 37.339 | 29.418 | 53.244 | 1.00 | 38.11 | A | C |
| ATOM | 1952 | CG1 | VAL | A | 261 | 37.634 | 27.949 | 52.965 | 1.00 | 36.67 | A | C |
| ATOM | 1953 | CG2 | VAL | A | 261 | 38.243 | 30.301 | 52.392 | 1.00 | 35.49 | A | C |
| ATOM | 1954 | C | VAL | A | 261 | 34.996 | 28.558 | 53.372 | 1.00 | 38.50 | A | C |
| ATOM | 1955 | O | VAL | A | 261 | 34.456 | 27.839 | 52.532 | 1.00 | 41.23 | A | O |
| ATOM | 1956 | N | GLU | A | 262 | 34.859 | 28.360 | 54.676 | 1.00 | 37.82 | A | N |
| ATOM | 1957 | CA | GLU | A | 262 | 34.087 | 27.233 | 55.180 | 1.00 | 40.00 | A | C |
| ATOM | 1958 | CB | GLU | A | 262 | 34.265 | 27.115 | 56.693 | 1.00 | 39.26 | A | C |
| ATOM | 1959 | CG | GLU | A | 262 | 35.709 | 26.932 | 57.097 | 1.00 | 39.77 | A | C |
| ATOM | 1960 | CD | GLU | A | 262 | 35.873 | 26.554 | 58.550 | 1.00 | 43.50 | A | C |
| ATOM | 1961 | OE1 | GLU | A | 262 | 37.024 | 26.281 | 58.957 | 1.00 | 41.93 | A | O |
| ATOM | 1962 | OE2 | GLU | A | 262 | 34.856 | 26.524 | 59.283 | 1.00 | 44.51 | A | O |
| ATOM | 1963 | C | GLU | A | 262 | 32.603 | 27.245 | 54.839 | 1.00 | 41.03 | A | C |
| ATOM | 1964 | O | GLU | A | 262 | 32.007 | 26.188 | 54.630 | 1.00 | 42.76 | A | O |
| ATOM | 1965 | N | SER | A | 263 | 32.005 | 28.430 | 54.785 | 1.00 | 41.08 | A | N |
| ATOM | 1966 | CA | SER | A | 263 | 30.585 | 28.542 | 54.473 | 1.00 | 41.47 | A | C |
| ATOM | 1967 | CB | SER | A | 263 | 30.035 | 29.885 | 54.973 | 1.00 | 41.33 | A | C |
| ATOM | 1968 | OG | SER | A | 263 | 30.637 | 30.980 | 54.302 | 1.00 | 40.39 | A | O |
| ATOM | 1969 | C | SER | A | 263 | 30.309 | 28.401 | 52.977 | 1.00 | 42.90 | A | C |
| ATOM | 1970 | O | SER | A | 263 | 29.153 | 28.390 | 52.561 | 1.00 | 43.89 | A | O |
| ATOM | 1971 | N | HIS | A | 264 | 31.367 | 28.296 | 52.174 | 1.00 | 43.31 | A | N |
| ATOM | 1972 | CA | HIS | A | 264 | 31.211 | 28.152 | 50.727 | 1.00 | 44.41 | A | C |
| ATOM | 1973 | CB | HIS | A | 264 | 31.708 | 29.401 | 50.007 | 1.00 | 40.41 | A | C |
| ATOM | 1974 | CG | HIS | A | 264 | 30.904 | 30.622 | 50.306 | 1.00 | 39.68 | A | C |
| ATOM | 1975 | CD2 | HIS | A | 264 | 30.090 | 31.369 | 49.524 | 1.00 | 37.15 | A | C |
| ATOM | 1976 | ND1 | HIS | A | 264 | 30.871 | 31.199 | 51.558 | 1.00 | 40.40 | A | N |
| ATOM | 1977 | CE1 | HIS | A | 264 | 30.071 | 32.249 | 51.534 | 1.00 | 38.85 | A | C |
| ATOM | 1978 | NE2 | HIS | A | 264 | 29.583 | 32.374 | 50.312 | 1.00 | 36.15 | A | N |
| ATOM | 1979 | C | HIS | A | 264 | 31.927 | 26.944 | 50.148 | 1.00 | 45.63 | A | C |
| ATOM | 1980 | O | HIS | A | 264 | 32.287 | 26.944 | 48.977 | 1.00 | 45.64 | A | O |
| ATOM | 1981 | N | GLU | A | 265 | 32.124 | 25.910 | 50.953 | 1.00 | 49.14 | A | N |
| ATOM | 1982 | CA | GLU | A | 265 | 32.812 | 24.726 | 50.463 | 1.00 | 54.81 | A | C |
| ATOM | 1983 | CB | GLU | A | 265 | 33.041 | 23.752 | 51.606 | 1.00 | 55.84 | A | C |
| ATOM | 1984 | CG | GLU | A | 265 | 33.747 | 24.405 | 52.756 | 1.00 | 61.36 | A | C |
| ATOM | 1985 | CD | GLU | A | 265 | 34.395 | 23.410 | 53.676 | 1.00 | 65.11 | A | C |
| ATOM | 1986 | OE1 | GLU | A | 265 | 33.669 | 22.543 | 54.217 | 1.00 | 65.74 | A | O |
| ATOM | 1987 | OE2 | GLU | A | 265 | 35.632 | 23.507 | 53.853 | 1.00 | 67.13 | A | O |
| ATOM | 1988 | C | GLU | A | 265 | 32.053 | 24.037 | 49.343 | 1.00 | 56.24 | A | C |
| ATOM | 1989 | O | GLU | A | 265 | 32.519 | 23.040 | 48.793 | 1.00 | 57.22 | A | O |
| ATOM | 1990 | N | SER | A | 266 | 30.889 | 24.584 | 49.002 | 1.00 | 58.31 | A | N |
| ATOM | 1991 | CA | SER | A | 266 | 30.058 | 24.021 | 47.950 | 1.00 | 59.27 | A | C |
| ATOM | 1992 | CB | SER | A | 266 | 28.806 | 23.387 | 48.563 | 1.00 | 58.79 | A | C |
| ATOM | 1993 | OG | SER | A | 266 | 29.139 | 22.385 | 49.509 | 1.00 | 59.56 | A | O |
| ATOM | 1994 | C | SER | A | 266 | 29.648 | 25.036 | 46.889 | 1.00 | 60.52 | A | C |
| ATOM | 1995 | O | SER | A | 266 | 28.493 | 25.046 | 46.460 | 1.00 | 62.89 | A | O |
| ATOM | 1996 | N | SER | A | 267 | 30.582 | 25.882 | 46.456 | 1.00 | 60.55 | A | N |
| ATOM | 1997 | CA | SER | A | 267 | 30.270 | 26.877 | 45.427 | 1.00 | 62.10 | A | C |
| ATOM | 1998 | CB | SER | A | 267 | 29.068 | 27.723 | 45.867 | 1.00 | 62.51 | A | C |
| ATOM | 1999 | OG | SER | A | 267 | 29.153 | 28.071 | 47.238 | 1.00 | 60.21 | A | O |
| ATOM | 2000 | C | SER | A | 267 | 31.436 | 27.788 | 45.048 | 1.00 | 63.35 | A | C |
| ATOM | 2001 | O | SER | A | 267 | 32.582 | 27.349 | 45.022 | 1.00 | 64.53 | A | O |
| ATOM | 2002 | N | LEU | A | 268 | 31.131 | 29.051 | 44.741 | 1.00 | 64.95 | A | N |
| ATOM | 2003 | CA | LEU | A | 268 | 32.142 | 30.046 | 44.363 | 1.00 | 64.71 | A | C |
| ATOM | 2004 | CB | LEU | A | 268 | 31.499 | 31.183 | 43.561 | 1.00 | 66.53 | A | C |
| ATOM | 2005 | CG | LEU | A | 268 | 30.546 | 32.124 | 44.307 | 1.00 | 68.83 | A | C |
| ATOM | 2006 | CD1 | LEU | A | 268 | 30.094 | 33.218 | 43.360 | 1.00 | 70.21 | A | C |
| ATOM | 2007 | CD2 | LEU | A | 268 | 29.349 | 31.355 | 44.860 | 1.00 | 70.48 | A | C |
| ATOM | 2008 | C | LEU | A | 268 | 32.857 | 30.611 | 45.604 | 1.00 | 64.10 | A | C |
| ATOM | 2009 | O | LEU | A | 268 | 33.396 | 29.829 | 46.391 | 1.00 | 66.40 | A | O |
| ATOM | 2010 | N | ILE | A | 269 | 32.859 | 31.941 | 45.790 | 1.00 | 58.59 | A | N |
| ATOM | 2011 | CA | ILE | A | 269 | 33.541 | 32.560 | 46.948 | 1.00 | 52.74 | A | C |
| ATOM | 2012 | CB | ILE | A | 269 | 34.984 | 32.012 | 47.054 | 1.00 | 51.78 | A | C |
| ATOM | 2013 | CG2 | ILE | A | 269 | 35.753 | 32.329 | 45.772 | 1.00 | 54.73 | A | C |
| ATOM | 2014 | CG1 | ILE | A | 269 | 35.680 | 32.561 | 48.292 | 1.00 | 50.48 | A | C |
| ATOM | 2015 | CD1 | ILE | A | 269 | 37.097 | 32.029 | 48.451 | 1.00 | 48.59 | A | C |
| ATOM | 2016 | C | ILE | A | 269 | 33.585 | 34.111 | 46.929 | 1.00 | 48.18 | A | C |
| ATOM | 2017 | O | ILE | A | 269 | 34.376 | 34.715 | 46.207 | 1.00 | 48.65 | A | O |
| ATOM | 2018 | N | PRO | A | 271 | 32.744 | 34.771 | 47.747 | 1.00 | 42.82 | A | N |
| ATOM | 2019 | CD | PRO | A | 271 | 31.695 | 34.184 | 48.598 | 1.00 | 40.01 | A | C |
| ATOM | 2020 | CA | PRO | A | 271 | 32.693 | 36.238 | 47.805 | 1.00 | 39.87 | A | C |
| ATOM | 2021 | CB | PRO | A | 271 | 31.365 | 36.507 | 48.502 | 1.00 | 37.73 | A | C |
| ATOM | 2022 | CG | PRO | A | 271 | 31.245 | 35.370 | 49.414 | 1.00 | 39.74 | A | C |
| ATOM | 2023 | C | PRO | A | 271 | 33.859 | 36.958 | 48.483 | 1.00 | 37.13 | A | C |
| ATOM | 2024 | O | PRO | A | 271 | 34.442 | 36.470 | 49.448 | 1.00 | 36.98 | A | O |
| ATOM | 2025 | N | PRO | A | 272 | 34.200 | 38.149 | 47.979 | 1.00 | 34.92 | A | N |
| ATOM | 2026 | CD | PRO | A | 272 | 33.606 | 38.784 | 46.788 | 1.00 | 34.38 | A | C |
| ATOM | 2027 | CA | PRO | A | 272 | 35.294 | 38.960 | 48.511 | 1.00 | 34.13 | A | C |
| ATOM | 2028 | CB | PRO | A | 272 | 35.520 | 39.994 | 47.412 | 1.00 | 32.34 | A | C |
| ATOM | 2029 | CG | PRO | A | 272 | 34.121 | 40.211 | 46.893 | 1.00 | 34.17 | A | C |
| ATOM | 2030 | C | PRO | A | 272 | 35.033 | 39.627 | 49.849 | 1.00 | 34.05 | A | C |
| ATOM | 2031 | O | PRO | A | 272 | 33.890 | 39.782 | 50.291 | 1.00 | 35.80 | A | O |
| ATOM | 2032 | N | ILE | A | 273 | 36.126 | 40.030 | 50.482 | 1.00 | 32.74 | A | N |
| ATOM | 2033 | CA | ILE | A | 273 | 36.067 | 40.723 | 51.748 | 1.00 | 31.00 | A | C |
| ATOM | 2034 | CB | ILE | A | 273 | 37.209 | 40.257 | 52.671 | 1.00 | 28.68 | A | C |
| ATOM | 2035 | CG2 | ILE | A | 273 | 37.262 | 41.115 | 53.910 | 1.00 | 26.28 | A | C |
| ATOM | 2036 | CG1 | ILE | A | 273 | 36.965 | 38.794 | 53.057 | 1.00 | 28.08 | A | C |
| ATOM | 2037 | CD1 | ILE | A | 273 | 37.959 | 38.207 | 54.028 | 1.00 | 28.06 | A | C |
| ATOM | 2038 | C | ILE | A | 273 | 36.186 | 42.198 | 51.390 | 1.00 | 32.36 | A | C |
| ATOM | 2039 | O | ILE | A | 273 | 37.147 | 42.616 | 50.732 | 1.00 | 32.43 | A | O |
| ATOM | 2040 | N | LYS | A | 274 | 35.176 | 42.974 | 51.777 | 1.00 | 32.87 | A | N |
| ATOM | 2041 | CA | LYS | A | 274 | 35.159 | 44.402 | 51.467 | 1.00 | 32.71 | A | C |
| ATOM | 2042 | CB | LYS | A | 274 | 33.789 | 44.798 | 50.906 | 1.00 | 33.80 | A | C |
| ATOM | 2043 | CG | LYS | A | 274 | 33.372 | 43.939 | 49.721 | 1.00 | 37.73 | A | C |
| ATOM | 2044 | CD | LYS | A | 274 | 32.104 | 44.429 | 49.035 | 1.00 | 41.74 | A | C |
| ATOM | 2045 | CE | LYS | A | 274 | 31.660 | 43.432 | 47.964 | 1.00 | 43.11 | A | C |
| ATOM | 2046 | NZ | LYS | A | 274 | 30.577 | 43.979 | 47.097 | 1.00 | 48.94 | A | N |
| ATOM | 2047 | C | LYS | A | 274 | 35.504 | 45.239 | 52.692 | 1.00 | 33.45 | A | C |
| ATOM | 2048 | O | LYS | A | 274 | 34.946 | 45.050 | 53.773 | 1.00 | 36.09 | A | O |
| ATOM | 2049 | N | VAL | A | 275 | 36.442 | 46.161 | 52.518 | 1.00 | 30.46 | A | N |
| ATOM | 2050 | CA | VAL | A | 275 | 36.884 | 47.009 | 53.606 | 1.00 | 26.60 | A | C |
| ATOM | 2051 | CB | VAL | A | 275 | 38.379 | 46.763 | 53.931 | 1.00 | 25.30 | A | C |
| ATOM | 2052 | CG1 | VAL | A | 275 | 38.874 | 47.804 | 54.903 | 1.00 | 21.42 | A | C |
| ATOM | 2053 | CG2 | VAL | A | 275 | 38.569 | 45.379 | 54.529 | 1.00 | 24.62 | A | C |
| ATOM | 2054 | C | VAL | A | 275 | 36.707 | 48.475 | 53.274 | 1.00 | 29.06 | A | C |
| ATOM | 2055 | O | VAL | A | 275 | 37.134 | 48.937 | 52.215 | 1.00 | 30.37 | A | O |
| ATOM | 2056 | N | MET | A | 276 | 36.073 | 49.208 | 54.181 | 1.00 | 28.77 | A | N |
| ATOM | 2057 | CA | MET | A | 276 | 35.887 | 50.628 | 53.983 | 1.00 | 27.88 | A | C |
| ATOM | 2058 | CB | MET | A | 276 | 34.412 | 51.023 | 54.048 | 1.00 | 31.99 | A | C |
| ATOM | 2059 | CG | MET | A | 276 | 34.209 | 52.523 | 53.807 | 1.00 | 37.30 | A | C |
| ATOM | 2060 | SD | MET | A | 276 | 32.509 | 53.069 | 53.753 | 1.00 | 44.43 | A | S |
| ATOM | 2061 | CE | MET | A | 276 | 32.508 | 54.084 | 55.209 | 1.00 | 46.76 | A | C |
| ATOM | 2062 | C | MET | A | 276 | 36.649 | 51.402 | 55.046 | 1.00 | 26.86 | A | C |
| ATOM | 2063 | O | MET | A | 276 | 36.576 | 51.094 | 56.233 | 1.00 | 26.76 | A | O |
| ATOM | 2064 | N | VAL | A | 277 | 37.391 | 52.405 | 54.595 | 1.00 | 24.78 | A | N |
| ATOM | 2065 | CA | VAL | A | 277 | 38.154 | 53.262 | 55.477 | 1.00 | 21.41 | A | C |
| ATOM | 2066 | CB | VAL | A | 277 | 39.648 | 53.310 | 55.060 | 1.00 | 21.13 | A | C |
| ATOM | 2067 | CG1 | VAL | A | 277 | 40.405 | 54.277 | 55.963 | 1.00 | 18.97 | A | C |
| ATOM | 2068 | CG2 | VAL | A | 277 | 40.260 | 51.901 | 55.146 | 1.00 | 16.06 | A | C |
| ATOM | 2069 | C | VAL | A | 277 | 37.517 | 54.643 | 55.352 | 1.00 | 23.62 | A | C |
| ATOM | 2070 | O | VAL | A | 277 | 37.611 | 55.287 | 54.305 | 1.00 | 24.39 | A | O |
| ATOM | 2071 | N | ALA | A | 278 | 36.855 | 55.086 | 56.419 | 1.00 | 23.15 | A | N |
| ATOM | 2072 | CA | ALA | A | 278 | 36.174 | 56.380 | 56.432 | 1.00 | 23.33 | A | C |
| ATOM | 2073 | CB | ALA | A | 278 | 34.687 | 56.205 | 56.811 | 1.00 | 17.97 | A | C |
| ATOM | 2074 | C | ALA | A | 278 | 36.832 | 57.333 | 57.405 | 1.00 | 23.28 | A | C |
| ATOM | 2075 | O | ALA | A | 278 | 37.128 | 56.964 | 58.533 | 1.00 | 26.07 | A | O |
| ATOM | 2076 | N | LEU | A | 279 | 37.058 | 58.562 | 56.963 | 1.00 | 23.14 | A | N |
| ATOM | 2077 | CA | LEU | A | 279 | 37.658 | 59.574 | 57.810 | 1.00 | 24.06 | A | C |
| ATOM | 2078 | CB | LEU | A | 279 | 38.915 | 60.137 | 57.152 | 1.00 | 24.20 | A | C |
| ATOM | 2079 | CG | LEU | A | 279 | 39.569 | 61.309 | 57.877 | 1.00 | 23.93 | A | C |
| ATOM | 2080 | CD1 | LEU | A | 279 | 39.982 | 60.865 | 59.265 | 1.00 | 25.63 | A | C |
| ATOM | 2081 | CD2 | LEU | A | 279 | 40.773 | 61.793 | 57.085 | 1.00 | 22.96 | A | C |
| ATOM | 2082 | C | LEU | A | 279 | 36.640 | 60.684 | 58.043 | 1.00 | 25.71 | A | C |
| ATOM | 2083 | O | LEU | A | 279 | 36.247 | 61.386 | 57.109 | 1.00 | 28.42 | A | O |
| ATOM | 2084 | N | GLY | A | 280 | 36.195 | 60.808 | 59.291 | 1.00 | 26.24 | A | N |
| ATOM | 2085 | CA | GLY | A | 280 | 35.227 | 61.825 | 59.652 | 1.00 | 26.82 | A | C |
| ATOM | 2086 | C | GLY | A | 280 | 35.920 | 62.920 | 60.439 | 1.00 | 29.23 | A | C |
| ATOM | 2087 | O | GLY | A | 280 | 37.143 | 62.897 | 60.572 | 1.00 | 29.70 | A | O |
| ATOM | 2088 | N | GLU | A | 281 | 35.155 | 63.880 | 60.955 | 1.00 | 31.29 | A | N |
| ATOM | 2089 | CA | GLU | A | 281 | 35.726 | 64.980 | 61.734 | 1.00 | 33.24 | A | C |
| ATOM | 2090 | CB | GLU | A | 281 | 34.629 | 65.970 | 62.146 | 1.00 | 35.85 | A | C |
| ATOM | 2091 | CG | GLU | A | 281 | 34.164 | 66.876 | 61.007 | 1.00 | 42.05 | A | C |
| ATOM | 2092 | CD | GLU | A | 281 | 35.324 | 67.583 | 60.318 | 1.00 | 44.34 | A | C |
| ATOM | 2093 | OE1 | GLU | A | 281 | 36.125 | 68.237 | 61.023 | 1.00 | 48.83 | A | O |
| ATOM | 2094 | OE2 | GLU | A | 281 | 35.434 | 67.485 | 59.075 | 1.00 | 45.17 | A | O |
| ATOM | 2095 | C | GLU | A | 281 | 36.480 | 64.490 | 62.970 | 1.00 | 32.68 | A | C |
| ATOM | 2096 | O | GLU | A | 281 | 37.489 | 65.073 | 63.356 | 1.00 | 32.53 | A | O |
| ATOM | 2097 | N | GLU | A | 282 | 35.997 | 63.432 | 63.610 | 1.00 | 32.17 | A | N |
| ATOM | 2098 | CA | GLU | A | 282 | 36.718 | 62.923 | 64.766 | 1.00 | 32.14 | A | C |
| ATOM | 2099 | CB | GLU | A | 282 | 36.014 | 63.305 | 66.067 | 1.00 | 32.05 | A | C |
| ATOM | 2100 | CG | GLU | A | 282 | 34.649 | 62.748 | 66.263 | 1.00 | 36.88 | A | C |
| ATOM | 2101 | CD | GLU | A | 282 | 34.072 | 63.170 | 67.610 | 1.00 | 42.23 | A | C |
| ATOM | 2102 | OE1 | GLU | A | 282 | 34.552 | 62.672 | 68.658 | 1.00 | 39.21 | A | O |
| ATOM | 2103 | OE2 | GLU | A | 282 | 33.145 | 64.013 | 67.616 | 1.00 | 43.54 | A | O |
| ATOM | 2104 | C | GLU | A | 282 | 36.994 | 61.421 | 64.727 | 1.00 | 30.85 | A | C |
| ATOM | 2105 | O | GLU | A | 282 | 37.895 | 60.941 | 65.414 | 1.00 | 30.60 | A | O |
| ATOM | 2106 | N | ASP | A | 283 | 36.239 | 60.684 | 63.919 | 1.00 | 27.56 | A | N |
| ATOM | 2107 | CA | ASP | A | 283 | 36.457 | 59.243 | 63.807 | 1.00 | 26.38 | A | C |
| ATOM | 2108 | CB | ASP | A | 283 | 35.142 | 58.457 | 63.808 | 1.00 | 23.92 | A | C |
| ATOM | 2109 | CG | ASP | A | 283 | 34.479 | 58.392 | 65.161 | 1.00 | 23.97 | A | C |
| ATOM | 2110 | OD1 | ASP | A | 283 | 35.083 | 58.813 | 66.168 | 1.00 | 25.27 | A | O |
| ATOM | 2111 | OD2 | ASP | A | 283 | 33.334 | 57.897 | 65.209 | 1.00 | 20.57 | A | O |
| ATOM | 2112 | C | ASP | A | 283 | 37.157 | 58.862 | 62.515 | 1.00 | 27.16 | A | C |
| ATOM | 2113 | O | ASP | A | 283 | 36.945 | 59.483 | 61.467 | 1.00 | 25.44 | A | O |
| ATOM | 2114 | N | LEU | A | 284 | 37.996 | 57.836 | 62.607 | 1.00 | 24.28 | A | N |
| ATOM | 2115 | CA | LEU | A | 284 | 38.653 | 57.255 | 61.445 | 1.00 | 23.86 | A | C |
| ATOM | 2116 | CB | LEU | A | 284 | 40.173 | 57.361 | 61.526 | 1.00 | 24.50 | A | C |
| ATOM | 2117 | CG | LEU | A | 284 | 40.877 | 57.109 | 60.179 | 1.00 | 28.18 | A | C |
| ATOM | 2118 | CD1 | LEU | A | 284 | 42.370 | 57.377 | 60.323 | 1.00 | 27.80 | A | C |
| ATOM | 2119 | CD2 | LEU | A | 284 | 40.634 | 55.680 | 59.701 | 1.00 | 27.18 | A | C |
| ATOM | 2120 | C | LEU | A | 284 | 38.193 | 55.813 | 61.645 | 1.00 | 23.17 | A | C |
| ATOM | 2121 | O | LEU | A | 284 | 38.680 | 55.111 | 62.528 | 1.00 | 24.00 | A | O |
| ATOM | 2122 | N | SER | A | 285 | 37.224 | 55.390 | 60.848 | 1.00 | 22.07 | A | N |
| ATOM | 2123 | CA | SER | A | 285 | 36.649 | 54.059 | 60.982 | 1.00 | 23.59 | A | C |
| ATOM | 2124 | CB | SER | A | 285 | 35.122 | 54.171 | 61.051 | 1.00 | 21.72 | A | C |
| ATOM | 2125 | OG | SER | A | 285 | 34.726 | 55.167 | 61.979 | 1.00 | 22.23 | A | O |
| ATOM | 2126 | C | SER | A | 285 | 37.023 | 53.084 | 59.882 | 1.00 | 23.75 | A | C |
| ATOM | 2127 | O | SER | A | 285 | 37.113 | 53.457 | 58.722 | 1.00 | 25.13 | A | O |
| ATOM | 2128 | N | ILE | A | 286 | 37.214 | 51.824 | 60.262 | 1.00 | 24.85 | A | N |
| ATOM | 2129 | CA | ILE | A | 286 | 37.563 | 50.766 | 59.320 | 1.00 | 24.09 | A | C |
| ATOM | 2130 | CB | ILE | A | 286 | 38.973 | 50.208 | 59.589 | 1.00 | 24.84 | A | C |
| ATOM | 2131 | CG2 | ILE | A | 286 | 39.373 | 49.249 | 58.483 | 1.00 | 19.97 | A | C |
| ATOM | 2132 | CG1 | ILE | A | 286 | 39.980 | 51.354 | 59.652 | 1.00 | 26.22 | A | C |
| ATOM | 2133 | CD1 | ILE | A | 286 | 41.327 | 50.943 | 60.199 | 1.00 | 28.24 | A | C |
| ATOM | 2134 | C | ILE | A | 286 | 36.590 | 49.602 | 59.431 | 1.00 | 25.51 | A | C |
| ATOM | 2135 | O | ILE | A | 286 | 36.598 | 48.869 | 60.423 | 1.00 | 25.04 | A | O |
| ATOM | 2136 | N | LYS | A | 287 | 35.747 | 49.432 | 58.421 | 1.00 | 26.29 | A | N |
| ATOM | 2137 | CA | LYS | A | 287 | 34.805 | 48.329 | 58.429 | 1.00 | 27.20 | A | C |
| ATOM | 2138 | CB | LYS | A | 287 | 33.424 | 48.787 | 57.953 | 1.00 | 27.96 | A | C |
| ATOM | 2139 | CG | LYS | A | 287 | 32.483 | 47.632 | 57.645 | 1.00 | 28.49 | A | C |
| ATOM | 2140 | CD | LYS | A | 287 | 31.142 | 47.767 | 58.342 | 1.00 | 31.00 | A | C |
| ATOM | 2141 | CE | LYS | A | 287 | 30.258 | 48.809 | 57.685 | 1.00 | 32.76 | A | C |
| ATOM | 2142 | NZ | LYS | A | 287 | 28.901 | 48.900 | 58.320 | 1.00 | 27.88 | A | N |
| ATOM | 2143 | C | LYS | A | 287 | 35.300 | 47.208 | 57.523 | 1.00 | 27.34 | A | C |
| ATOM | 2144 | O | LYS | A | 287 | 35.760 | 47.451 | 56.409 | 1.00 | 29.28 | A | O |
| ATOM | 2145 | N | MET | A | 288 | 35.220 | 45.980 | 58.016 | 1.00 | 26.03 | A | N |
| ATOM | 2146 | CA | MET | A | 288 | 35.600 | 44.812 | 57.239 | 1.00 | 25.77 | A | C |
| ATOM | 2147 | CB | MET | A | 288 | 36.724 | 44.029 | 57.927 | 1.00 | 25.54 | A | C |
| ATOM | 2148 | CG | MET | A | 288 | 37.189 | 42.801 | 57.148 | 1.00 | 23.07 | A | C |
| ATOM | 2149 | SD | MET | A | 288 | 38.436 | 41.846 | 58.025 | 1.00 | 28.76 | A | S |
| ATOM | 2150 | CE | MET | A | 288 | 39.781 | 43.075 | 58.223 | 1.00 | 25.07 | A | C |
| ATOM | 2151 | C | MET | A | 288 | 34.340 | 43.952 | 57.146 | 1.00 | 26.34 | A | C |
| ATOM | 2152 | O | MET | A | 288 | 33.886 | 43.400 | 58.149 | 1.00 | 27.13 | A | O |
| ATOM | 2153 | N | SER | A | 289 | 33.781 | 43.857 | 55.939 | 1.00 | 26.98 | A | N |
| ATOM | 2154 | CA | SER | A | 289 | 32.566 | 43.087 | 55.671 | 1.00 | 29.41 | A | C |
| ATOM | 2155 | CB | SER | A | 289 | 31.591 | 43.904 | 54.817 | 1.00 | 28.33 | A | C |
| ATOM | 2156 | OG | SER | A | 289 | 31.285 | 45.136 | 55.433 | 1.00 | 29.82 | A | O |
| ATOM | 2157 | C | SER | A | 289 | 32.836 | 41.790 | 54.936 | 1.00 | 29.88 | A | C |
| ATOM | 2158 | O | SER | A | 289 | 33.519 | 41.785 | 53.915 | 1.00 | 30.66 | A | O |
| ATOM | 2159 | N | ASP | A | 290 | 32.269 | 40.698 | 55.442 | 1.00 | 30.14 | A | N |
| ATOM | 2160 | CA | ASP | A | 290 | 32.417 | 39.391 | 54.813 | 1.00 | 30.95 | A | C |
| ATOM | 2161 | CB | ASP | A | 290 | 33.292 | 38.475 | 55.675 | 1.00 | 30.06 | A | C |
| ATOM | 2162 | CG | ASP | A | 290 | 32.618 | 38.066 | 56.997 | 1.00 | 30.88 | A | C |
| ATOM | 2163 | OD1 | ASP | A | 290 | 31.536 | 37.430 | 56.985 | 1.00 | 27.70 | A | O |
| ATOM | 2164 | OD2 | ASP | A | 290 | 33.189 | 38.375 | 58.065 | 1.00 | 29.49 | A | O |
| ATOM | 2165 | C | ASP | A | 290 | 31.046 | 38.745 | 54.633 | 1.00 | 32.42 | A | C |
| ATOM | 2166 | O | ASP | A | 290 | 30.077 | 39.135 | 55.292 | 1.00 | 32.56 | A | O |
| ATOM | 2167 | N | ARG | A | 291 | 30.964 | 37.768 | 53.734 | 1.00 | 32.18 | A | N |
| ATOM | 2168 | CA | ARG | A | 291 | 29.724 | 37.034 | 53.515 | 1.00 | 33.57 | A | C |
| ATOM | 2169 | CB | ARG | A | 291 | 29.323 | 37.061 | 52.037 | 1.00 | 38.04 | A | C |
| ATOM | 2170 | CG | ARG | A | 291 | 28.832 | 38.423 | 51.595 | 1.00 | 43.38 | A | C |
| ATOM | 2171 | CD | ARG | A | 291 | 28.411 | 38.463 | 50.140 | 1.00 | 51.40 | A | C |
| ATOM | 2172 | NE | ARG | A | 291 | 28.070 | 39.834 | 49.763 | 1.00 | 58.51 | A | N |
| ATOM | 2173 | CZ | ARG | A | 291 | 28.905 | 40.869 | 49.852 | 1.00 | 62.62 | A | C |
| ATOM | 2174 | NH1 | ARG | A | 291 | 30.149 | 40.700 | 50.301 | 1.00 | 62.38 | A | N |
| ATOM | 2175 | NH2 | ARG | A | 291 | 28.488 | 42.084 | 49.512 | 1.00 | 64.89 | A | N |
| ATOM | 2176 | C | ARG | A | 291 | 30.029 | 35.616 | 53.967 | 1.00 | 31.59 | A | C |
| ATOM | 2177 | O | ARG | A | 291 | 29.776 | 34.647 | 53.254 | 1.00 | 31.52 | A | O |
| ATOM | 2178 | N | GLY | A | 292 | 30.581 | 35.521 | 55.174 | 1.00 | 30.07 | A | N |
| ATOM | 2179 | CA | GLY | A | 292 | 30.974 | 34.240 | 55.731 | 1.00 | 27.46 | A | C |
| ATOM | 2180 | C | GLY | A | 292 | 29.885 | 33.449 | 56.419 | 1.00 | 27.37 | A | C |
| ATOM | 2181 | O | GLY | A | 292 | 30.181 | 32.520 | 57.173 | 1.00 | 26.35 | A | O |
| ATOM | 2182 | N | GLY | A | 293 | 28.627 | 33.808 | 56.171 | 1.00 | 26.82 | A | N |
| ATOM | 2183 | CA | GLY | A | 293 | 27.533 | 33.075 | 56.781 | 1.00 | 27.16 | A | C |
| ATOM | 2184 | C | GLY | A | 293 | 26.970 | 33.743 | 58.021 | 1.00 | 29.61 | A | C |
| ATOM | 2185 | O | GLY | A | 293 | 25.819 | 33.515 | 58.394 | 1.00 | 31.50 | A | O |
| ATOM | 2186 | N | GLY | A | 294 | 27.777 | 34.568 | 58.672 | 1.00 | 27.26 | A | N |
| ATOM | 2187 | CA | GLY | A | 294 | 27.298 | 35.246 | 59.854 | 1.00 | 27.83 | A | C |
| ATOM | 2188 | C | GLY | A | 294 | 27.263 | 34.356 | 61.074 | 1.00 | 30.11 | A | C |
| ATOM | 2189 | O | GLY | A | 294 | 27.592 | 33.171 | 61.009 | 1.00 | 30.70 | A | O |
| ATOM | 2190 | N | VAL | A | 295 | 26.862 | 34.941 | 62.198 | 1.00 | 29.20 | A | N |
| ATOM | 2191 | CA | VAL | A | 295 | 26.772 | 34.231 | 63.465 | 1.00 | 28.14 | A | C |
| ATOM | 2192 | CB | VAL | A | 295 | 28.039 | 34.506 | 64.323 | 1.00 | 29.06 | A | C |
| ATOM | 2193 | CG1 | VAL | A | 295 | 27.837 | 34.032 | 65.739 | 1.00 | 29.07 | A | C |
| ATOM | 2194 | CG2 | VAL | A | 295 | 29.233 | 33.783 | 63.721 | 1.00 | 27.58 | A | C |
| ATOM | 2195 | C | VAL | A | 295 | 25.516 | 34.725 | 64.193 | 1.00 | 30.65 | A | C |
| ATOM | 2196 | O | VAL | A | 295 | 25.174 | 35.903 | 64.100 | 1.00 | 29.11 | A | O |
| ATOM | 2197 | N | PRO | A | 296 | 24.804 | 33.829 | 64.915 | 1.00 | 29.52 | A | N |
| ATOM | 2198 | CD | PRO | A | 296 | 25.170 | 32.449 | 65.281 | 1.00 | 28.40 | A | C |
| ATOM | 2199 | CA | PRO | A | 296 | 23.594 | 34.247 | 65.632 | 1.00 | 29.44 | A | C |
| ATOM | 2200 | CB | PRO | A | 296 | 23.076 | 32.944 | 66.267 | 1.00 | 29.81 | A | C |
| ATOM | 2201 | CG | PRO | A | 296 | 23.843 | 31.845 | 65.584 | 1.00 | 30.76 | A | C |
| ATOM | 2202 | C | PRO | A | 296 | 23.967 | 35.271 | 66.698 | 1.00 | 31.72 | A | C |
| ATOM | 2203 | O | PRO | A | 296 | 25.011 | 35.155 | 67.348 | 1.00 | 31.38 | A | O |
| ATOM | 2204 | N | LEU | A | 297 | 23.112 | 36.264 | 66.888 | 1.00 | 31.22 | A | N |
| ATOM | 2205 | CA | LEU | A | 297 | 23.381 | 37.300 | 67.877 | 1.00 | 32.00 | A | C |
| ATOM | 2206 | CB | LEU | A | 297 | 22.148 | 38.195 | 68.060 | 1.00 | 29.77 | A | C |
| ATOM | 2207 | CG | LEU | A | 297 | 22.320 | 39.361 | 69.051 | 1.00 | 29.19 | A | C |
| ATOM | 2208 | CD1 | LEU | A | 297 | 23.486 | 40.255 | 68.641 | 1.00 | 25.52 | A | C |
| ATOM | 2209 | CD2 | LEU | A | 297 | 21.036 | 40.176 | 69.120 | 1.00 | 28.35 | A | C |
| ATOM | 2210 | C | LEU | A | 297 | 23.841 | 36.789 | 69.244 | 1.00 | 33.28 | A | C |
| ATOM | 2211 | O | LEU | A | 297 | 24.718 | 37.385 | 69.850 | 1.00 | 32.37 | A | O |
| ATOM | 2212 | N | ARG | A | 298 | 23.260 | 35.696 | 69.737 | 1.00 | 37.27 | A | N |
| ATOM | 2213 | CA | ARG | A | 298 | 23.658 | 35.189 | 71.053 | 1.00 | 38.80 | A | C |
| ATOM | 2214 | CB | ARG | A | 298 | 22.705 | 34.083 | 71.542 | 1.00 | 42.97 | A | C |
| ATOM | 2215 | CG | ARG | A | 298 | 22.440 | 32.944 | 70.572 | 1.00 | 49.19 | A | C |
| ATOM | 2216 | CD | ARG | A | 298 | 21.631 | 31.841 | 71.252 | 1.00 | 52.93 | A | C |
| ATOM | 2217 | NE | ARG | A | 298 | 22.494 | 30.795 | 71.789 | 1.00 | 55.40 | A | N |
| ATOM | 2218 | CZ | ARG | A | 298 | 23.069 | 29.850 | 71.049 | 1.00 | 57.62 | A | C |
| ATOM | 2219 | NH1 | ARG | A | 298 | 22.870 | 29.812 | 69.738 | 1.00 | 56.74 | A | N |
| ATOM | 2220 | NH2 | ARG | A | 298 | 23.856 | 28.948 | 71.621 | 1.00 | 60.12 | A | N |
| ATOM | 2221 | C | ARG | A | 298 | 25.092 | 34.698 | 71.176 | 1.00 | 37.52 | A | C |
| ATOM | 2222 | O | ARG | A | 298 | 25.660 | 34.734 | 72.262 | 1.00 | 38.91 | A | O |
| ATOM | 2223 | N | LYS | A | 299 | 25.691 | 34.238 | 70.084 | 1.00 | 38.07 | A | N |
| ATOM | 2224 | CA | LYS | A | 299 | 27.066 | 33.755 | 70.164 | 1.00 | 37.87 | A | C |
| ATOM | 2225 | CB | LYS | A | 299 | 27.333 | 32.685 | 69.092 | 1.00 | 38.72 | A | C |
| ATOM | 2226 | CG | LYS | A | 299 | 26.477 | 31.432 | 69.289 | 1.00 | 45.06 | A | C |
| ATOM | 2227 | CD | LYS | A | 299 | 26.807 | 30.298 | 68.322 | 1.00 | 48.01 | A | C |
| ATOM | 2228 | CE | LYS | A | 299 | 25.711 | 29.224 | 68.401 | 1.00 | 55.41 | A | C |
| ATOM | 2229 | NZ | LYS | A | 299 | 26.016 | 27.966 | 67.637 | 1.00 | 56.58 | A | N |
| ATOM | 2230 | C | LYS | A | 299 | 28.063 | 34.900 | 70.047 | 1.00 | 36.42 | A | C |
| ATOM | 2231 | O | LYS | A | 299 | 29.199 | 34.795 | 70.504 | 1.00 | 34.71 | A | O |
| ATOM | 2232 | N | ILE | A | 300 | 27.628 | 36.005 | 69.455 | 1.00 | 38.69 | A | N |
| ATOM | 2233 | CA | ILE | A | 300 | 28.502 | 37.166 | 69.294 | 1.00 | 41.22 | A | C |
| ATOM | 2234 | CB | ILE | A | 300 | 27.736 | 38.372 | 68.698 | 1.00 | 41.46 | A | C |
| ATOM | 2235 | CG2 | ILE | A | 300 | 28.578 | 39.634 | 68.836 | 1.00 | 39.84 | A | C |
| ATOM | 2236 | CG1 | ILE | A | 300 | 27.384 | 38.103 | 67.232 | 1.00 | 43.20 | A | C |
| ATOM | 2237 | CD1 | ILE | A | 300 | 28.607 | 37.854 | 66.349 | 1.00 | 47.20 | A | C |
| ATOM | 2238 | C | ILE | A | 300 | 29.088 | 37.582 | 70.640 | 1.00 | 41.96 | A | C |
| ATOM | 2239 | O | ILE | A | 300 | 30.293 | 37.790 | 70.787 | 1.00 | 41.20 | A | O |
| ATOM | 2240 | N | GLU | A | 301 | 28.210 | 37.692 | 71.622 | 1.00 | 45.64 | A | N |
| ATOM | 2241 | CA | GLU | A | 301 | 28.578 | 38.087 | 72.970 | 1.00 | 49.00 | A | C |
| ATOM | 2242 | CB | GLU | A | 301 | 27.327 | 38.003 | 73.849 | 1.00 | 54.75 | A | C |
| ATOM | 2243 | CG | GLU | A | 301 | 25.994 | 38.017 | 73.039 | 1.00 | 59.49 | A | C |
| ATOM | 2244 | CD | GLU | A | 301 | 25.540 | 39.415 | 72.598 | 1.00 | 64.16 | A | C |
| ATOM | 2245 | OE1 | GLU | A | 301 | 26.349 | 40.157 | 72.001 | 1.00 | 65.45 | A | O |
| ATOM | 2246 | OE2 | GLU | A | 301 | 24.364 | 39.772 | 72.849 | 1.00 | 67.79 | A | O |
| ATOM | 2247 | C | GLU | A | 301 | 29.699 | 37.201 | 73.535 | 1.00 | 49.26 | A | C |
| ATOM | 2248 | O | GLU | A | 301 | 30.561 | 37.677 | 74.268 | 1.00 | 50.56 | A | O |
| ATOM | 2249 | N | ARG | A | 302 | 29.695 | 35.919 | 73.182 | 1.00 | 47.53 | A | N |
| ATOM | 2250 | CA | ARG | A | 302 | 30.702 | 34.973 | 73.678 | 1.00 | 45.62 | A | C |
| ATOM | 2251 | CB | ARG | A | 302 | 30.156 | 33.543 | 73.579 | 1.00 | 48.72 | A | C |
| ATOM | 2252 | CG | ARG | A | 302 | 28.726 | 33.406 | 74.093 | 1.00 | 55.42 | A | C |
| ATOM | 2253 | CD | ARG | A | 302 | 28.186 | 32.010 | 73.840 | 1.00 | 58.76 | A | C |
| ATOM | 2254 | NE | ARG | A | 302 | 28.850 | 31.007 | 74.669 | 1.00 | 63.55 | A | N |
| ATOM | 2255 | CZ | ARG | A | 302 | 28.689 | 30.900 | 75.986 | 1.00 | 64.35 | A | C |
| ATOM | 2256 | NH1 | ARG | A | 302 | 27.882 | 31.737 | 76.633 | 1.00 | 63.83 | A | N |
| ATOM | 2257 | NH2 | ARG | A | 302 | 29.335 | 29.957 | 76.657 | 1.00 | 63.97 | A | N |
| ATOM | 2258 | C | ARG | A | 302 | 32.045 | 35.055 | 72.943 | 1.00 | 42.51 | A | C |
| ATOM | 2259 | O | ARG | A | 302 | 33.080 | 34.619 | 73.457 | 1.00 | 40.97 | A | O |
| ATOM | 2260 | N | LEU | A | 303 | 32.026 | 35.610 | 71.738 | 1.00 | 38.94 | A | N |
| ATOM | 2261 | CA | LEU | A | 303 | 33.239 | 35.751 | 70.947 | 1.00 | 36.26 | A | C |
| ATOM | 2262 | CB | LEU | A | 303 | 32.901 | 36.272 | 69.539 | 1.00 | 35.19 | A | C |
| ATOM | 2263 | CG | LEU | A | 303 | 32.253 | 35.301 | 68.548 | 1.00 | 33.05 | A | C |
| ATOM | 2264 | CD1 | LEU | A | 303 | 31.750 | 36.044 | 67.319 | 1.00 | 32.27 | A | C |
| ATOM | 2265 | CD2 | LEU | A | 303 | 33.263 | 34.253 | 68.162 | 1.00 | 28.63 | A | C |
| ATOM | 2266 | C | LEU | A | 303 | 34.263 | 36.683 | 71.600 | 1.00 | 34.96 | A | C |
| ATOM | 2267 | O | LEU | A | 303 | 35.447 | 36.601 | 71.297 | 1.00 | 35.58 | A | O |
| ATOM | 2268 | N | PHE | A | 304 | 33.822 | 37.569 | 72.488 | 1.00 | 33.38 | A | N |
| ATOM | 2269 | CA | PHE | A | 304 | 34.761 | 38.487 | 73.127 | 1.00 | 33.13 | A | C |
| ATOM | 2270 | CB | PHE | A | 304 | 34.180 | 39.910 | 73.174 | 1.00 | 32.98 | A | C |
| ATOM | 2271 | CG | PHE | A | 304 | 34.252 | 40.642 | 71.856 | 1.00 | 30.66 | A | C |
| ATOM | 2272 | CD1 | PHE | A | 304 | 33.204 | 40.584 | 70.954 | 1.00 | 30.27 | A | C |
| ATOM | 2273 | CD2 | PHE | A | 304 | 35.383 | 41.368 | 71.512 | 1.00 | 31.58 | A | C |
| ATOM | 2274 | CE1 | PHE | A | 304 | 33.281 | 41.239 | 69.732 | 1.00 | 30.79 | A | C |
| ATOM | 2275 | CE2 | PHE | A | 304 | 35.468 | 42.024 | 70.292 | 1.00 | 32.50 | A | C |
| ATOM | 2276 | CZ | PHE | A | 304 | 34.413 | 41.959 | 69.403 | 1.00 | 29.75 | A | C |
| ATOM | 2277 | C | PHE | A | 304 | 35.176 | 38.050 | 74.529 | 1.00 | 33.32 | A | C |
| ATOM | 2278 | O | PHE | A | 304 | 36.019 | 38.685 | 75.161 | 1.00 | 33.78 | A | O |
| ATOM | 2279 | N | SER | A | 305 | 34.597 | 36.952 | 75.002 | 1.00 | 32.61 | A | N |
| ATOM | 2280 | CA | SER | A | 305 | 34.886 | 36.433 | 76.334 | 1.00 | 32.46 | A | C |
| ATOM | 2281 | CB | SER | A | 305 | 33.673 | 35.655 | 76.845 | 1.00 | 33.59 | A | C |
| ATOM | 2282 | OG | SER | A | 305 | 34.051 | 34.751 | 77.862 | 1.00 | 36.02 | A | O |
| ATOM | 2283 | C | SER | A | 305 | 36.126 | 35.544 | 76.423 | 1.00 | 31.98 | A | C |
| ATOM | 2284 | O | SER | A | 305 | 36.242 | 34.550 | 75.720 | 1.00 | 30.75 | A | O |
| ATOM | 2285 | N | TYR | A | 306 | 37.051 | 35.894 | 77.306 | 1.00 | 32.75 | A | N |
| ATOM | 2286 | CA | TYR | A | 306 | 38.247 | 35.083 | 77.466 | 1.00 | 33.95 | A | C |
| ATOM | 2287 | CB | TYR | A | 306 | 39.163 | 35.659 | 78.545 | 1.00 | 31.84 | A | C |
| ATOM | 2288 | CG | TYR | A | 306 | 39.679 | 37.031 | 78.226 | 1.00 | 30.52 | A | C |
| ATOM | 2289 | CD1 | TYR | A | 306 | 39.124 | 38.152 | 78.832 | 1.00 | 28.43 | A | C |
| ATOM | 2290 | CE1 | TYR | A | 306 | 39.555 | 39.425 | 78.508 | 1.00 | 27.57 | A | C |
| ATOM | 2291 | CD2 | TYR | A | 306 | 40.695 | 37.218 | 77.277 | 1.00 | 27.54 | A | C |
| ATOM | 2292 | CE2 | TYR | A | 306 | 41.135 | 38.493 | 76.940 | 1.00 | 25.87 | A | C |
| ATOM | 2293 | CZ | TYR | A | 306 | 40.555 | 39.595 | 77.562 | 1.00 | 27.60 | A | C |
| ATOM | 2294 | OH | TYR | A | 306 | 40.951 | 40.876 | 77.243 | 1.00 | 24.64 | A | O |
| ATOM | 2295 | C | TYR | A | 306 | 37.877 | 33.669 | 77.871 | 1.00 | 35.48 | A | C |
| ATOM | 2296 | O | TYR | A | 306 | 38.426 | 32.700 | 77.347 | 1.00 | 34.83 | A | O |
| ATOM | 2297 | N | MET | A | 307 | 36.939 | 33.559 | 78.807 | 1.00 | 38.14 | A | N |
| ATOM | 2298 | CA | MET | A | 307 | 36.528 | 32.260 | 79.318 | 1.00 | 40.87 | A | C |
| ATOM | 2299 | CB | MET | A | 307 | 35.686 | 32.433 | 80.580 | 1.00 | 40.93 | A | C |
| ATOM | 2300 | CG | MET | A | 307 | 35.286 | 31.119 | 81.218 | 1.00 | 43.57 | A | C |
| ATOM | 2301 | SD | MET | A | 307 | 34.394 | 31.395 | 82.730 | 1.00 | 46.35 | A | S |
| ATOM | 2302 | CE | MET | A | 307 | 32.759 | 31.494 | 82.113 | 1.00 | 41.84 | A | C |
| ATOM | 2303 | C | MET | A | 307 | 35.774 | 31.404 | 78.320 | 1.00 | 41.92 | A | C |
| ATOM | 2304 | O | MET | A | 307 | 36.204 | 30.291 | 78.015 | 1.00 | 40.77 | A | O |
| ATOM | 2305 | N | TYR | A | 308 | 34.654 | 31.915 | 77.814 | 1.00 | 44.66 | A | N |
| ATOM | 2306 | CA | TYR | A | 308 | 33.847 | 31.161 | 76.858 | 1.00 | 47.05 | A | C |
| ATOM | 2307 | CB | TYR | A | 308 | 32.563 | 31.935 | 76.501 | 1.00 | 47.25 | A | C |
| ATOM | 2308 | CG | TYR | A | 308 | 31.596 | 32.088 | 77.660 | 1.00 | 48.28 | A | C |
| ATOM | 2309 | CD1 | TYR | A | 308 | 30.826 | 33.246 | 77.805 | 1.00 | 49.44 | A | C |
| ATOM | 2310 | CE1 | TYR | A | 308 | 29.971 | 33.409 | 78.890 | 1.00 | 51.89 | A | C |
| ATOM | 2311 | CD2 | TYR | A | 308 | 31.473 | 31.091 | 78.628 | 1.00 | 48.53 | A | C |
| ATOM | 2312 | CE2 | TYR | A | 308 | 30.618 | 31.245 | 79.711 | 1.00 | 51.50 | A | C |
| ATOM | 2313 | CZ | TYR | A | 308 | 29.871 | 32.407 | 79.838 | 1.00 | 52.96 | A | C |
| ATOM | 2314 | OH | TYR | A | 308 | 29.028 | 32.577 | 80.916 | 1.00 | 54.25 | A | O |
| ATOM | 2315 | C | TYR | A | 308 | 34.639 | 30.831 | 75.595 | 1.00 | 47.69 | A | C |
| ATOM | 2316 | O | TYR | A | 308 | 34.260 | 29.936 | 74.847 | 1.00 | 49.54 | A | O |
| ATOM | 2317 | N | SER | A | 309 | 35.748 | 31.530 | 75.368 | 1.00 | 48.47 | A | N |
| ATOM | 2318 | CA | SER | A | 309 | 36.543 | 31.271 | 74.174 | 1.00 | 51.10 | A | C |
| ATOM | 2319 | CB | SER | A | 309 | 36.757 | 32.569 | 73.376 | 1.00 | 49.14 | A | C |
| ATOM | 2320 | OG | SER | A | 309 | 37.649 | 33.454 | 74.026 | 1.00 | 47.32 | A | O |
| ATOM | 2321 | C | SER | A | 309 | 37.884 | 30.621 | 74.507 | 1.00 | 53.64 | A | C |
| ATOM | 2322 | O | SER | A | 309 | 38.908 | 30.934 | 73.900 | 1.00 | 53.31 | A | O |
| ATOM | 2323 | N | THR | A | 310 | 37.861 | 29.709 | 75.478 | 1.00 | 57.59 | A | N |
| ATOM | 2324 | CA | THR | A | 310 | 39.057 | 28.988 | 75.914 | 1.00 | 60.04 | A | C |
| ATOM | 2325 | CB | THR | A | 310 | 39.490 | 29.444 | 77.325 | 1.00 | 59.41 | A | C |
| ATOM | 2326 | OG1 | THR | A | 310 | 39.840 | 30.830 | 77.284 | 1.00 | 55.94 | A | O |
| ATOM | 2327 | CG2 | THR | A | 310 | 40.693 | 28.632 | 77.818 | 1.00 | 59.14 | A | C |
| ATOM | 2328 | C | THR | A | 310 | 38.775 | 27.484 | 75.936 | 1.00 | 62.84 | A | C |
| ATOM | 2329 | O | THR | A | 310 | 37.678 | 27.058 | 76.298 | 1.00 | 63.55 | A | O |
| ATOM | 2330 | N | ALA | A | 311 | 39.768 | 26.687 | 75.548 | 1.00 | 66.87 | A | N |
| ATOM | 2331 | CA | ALA | A | 311 | 39.622 | 25.229 | 75.510 | 1.00 | 70.53 | A | C |
| ATOM | 2332 | CB | ALA | A | 311 | 38.904 | 24.813 | 74.225 | 1.00 | 69.65 | A | C |
| ATOM | 2333 | C | ALA | A | 311 | 40.993 | 24.549 | 75.595 | 1.00 | 72.87 | A | C |
| ATOM | 2334 | O | ALA | A | 311 | 42.028 | 25.213 | 75.519 | 1.00 | 72.45 | A | O |
| ATOM | 2335 | N | PRO | A | 312 | 41.019 | 23.206 | 75.736 | 1.00 | 75.70 | A | N |
| ATOM | 2336 | CD | PRO | A | 312 | 42.295 | 22.477 | 75.654 | 1.00 | 76.67 | A | C |
| ATOM | 2337 | CA | PRO | A | 312 | 39.892 | 22.264 | 75.835 | 1.00 | 76.88 | A | C |
| ATOM | 2338 | CB | PRO | A | 312 | 40.593 | 20.908 | 75.909 | 1.00 | 77.11 | A | C |
| ATOM | 2339 | CG | PRO | A | 312 | 41.853 | 21.138 | 75.129 | 1.00 | 77.80 | A | C |
| ATOM | 2340 | C | PRO | A | 312 | 38.978 | 22.502 | 77.035 | 1.00 | 78.44 | A | C |
| ATOM | 2341 | O | PRO | A | 312 | 37.813 | 22.099 | 77.030 | 1.00 | 79.60 | A | O |
| ATOM | 2342 | N | GLY | A | 327 | 41.150 | 31.370 | 70.306 | 1.00 | 56.03 | A | N |
| ATOM | 2343 | CA | GLY | A | 327 | 42.120 | 32.122 | 71.084 | 1.00 | 53.04 | A | C |
| ATOM | 2344 | C | GLY | A | 327 | 41.572 | 33.460 | 71.547 | 1.00 | 51.80 | A | C |
| ATOM | 2345 | O | GLY | A | 327 | 40.365 | 33.709 | 71.455 | 1.00 | 51.57 | A | O |
| ATOM | 2346 | N | TYR | A | 328 | 42.458 | 34.328 | 72.041 | 1.00 | 48.94 | A | N |
| ATOM | 2347 | CA | TYR | A | 328 | 42.056 | 35.654 | 72.525 | 1.00 | 45.66 | A | C |
| ATOM | 2348 | CB | TYR | A | 328 | 42.795 | 35.993 | 73.823 | 1.00 | 44.99 | A | C |
| ATOM | 2349 | CG | TYR | A | 328 | 42.512 | 35.051 | 74.968 | 1.00 | 46.55 | A | C |
| ATOM | 2350 | CD1 | TYR | A | 328 | 41.347 | 34.276 | 75.001 | 1.00 | 47.40 | A | C |
| ATOM | 2351 | CE1 | TYR | A | 328 | 41.084 | 33.419 | 76.071 | 1.00 | 48.69 | A | C |
| ATOM | 2352 | CD2 | TYR | A | 328 | 43.406 | 34.947 | 76.037 | 1.00 | 47.59 | A | C |
| ATOM | 2353 | CE2 | TYR | A | 328 | 43.153 | 34.101 | 77.111 | 1.00 | 46.96 | A | C |
| ATOM | 2354 | CZ | TYR | A | 328 | 41.994 | 33.340 | 77.124 | 1.00 | 47.53 | A | C |
| ATOM | 2355 | OH | TYR | A | 328 | 41.746 | 32.503 | 78.187 | 1.00 | 47.27 | A | O |
| ATOM | 2356 | C | TYR | A | 328 | 42.258 | 36.800 | 71.517 | 1.00 | 42.31 | A | C |
| ATOM | 2357 | O | TYR | A | 328 | 42.323 | 37.972 | 71.900 | 1.00 | 40.97 | A | O |
| ATOM | 2358 | N | GLY | A | 329 | 42.333 | 36.453 | 70.233 | 1.00 | 37.67 | A | N |
| ATOM | 2359 | CA | GLY | A | 329 | 42.526 | 37.450 | 69.196 | 1.00 | 33.55 | A | C |
| ATOM | 2360 | C | GLY | A | 329 | 41.538 | 38.609 | 69.210 | 1.00 | 32.39 | A | C |
| ATOM | 2361 | O | GLY | A | 329 | 41.940 | 39.766 | 69.140 | 1.00 | 32.44 | A | O |
| ATOM | 2362 | N | LEU | A | 330 | 40.245 | 38.309 | 69.296 | 1.00 | 30.57 | A | N |
| ATOM | 2363 | CA | LEU | A | 330 | 39.225 | 39.351 | 69.303 | 1.00 | 29.58 | A | C |
| ATOM | 2364 | CB | LEU | A | 330 | 37.830 | 38.734 | 69.136 | 1.00 | 30.11 | A | C |
| ATOM | 2365 | CG | LEU | A | 330 | 37.434 | 38.455 | 67.684 | 1.00 | 31.69 | A | C |
| ATOM | 2366 | CD1 | LEU | A | 330 | 36.123 | 37.671 | 67.617 | 1.00 | 28.32 | A | C |
| ATOM | 2367 | CD2 | LEU | A | 330 | 37.304 | 39.799 | 66.952 | 1.00 | 30.92 | A | C |
| ATOM | 2368 | C | LEU | A | 330 | 39.261 | 40.228 | 70.556 | 1.00 | 30.84 | A | C |
| ATOM | 2369 | O | LEU | A | 330 | 39.428 | 41.455 | 70.457 | 1.00 | 27.80 | A | O |
| ATOM | 2370 | N | PRO | A | 331 | 39.111 | 39.620 | 71.750 | 1.00 | 27.54 | A | N |
| ATOM | 2371 | CD | PRO | A | 331 | 38.867 | 38.211 | 72.106 | 1.00 | 28.21 | A | C |
| ATOM | 2372 | CA | PRO | A | 331 | 39.144 | 40.467 | 72.947 | 1.00 | 26.07 | A | C |
| ATOM | 2373 | CB | PRO | A | 331 | 38.982 | 39.466 | 74.103 | 1.00 | 27.82 | A | C |
| ATOM | 2374 | CG | PRO | A | 331 | 39.411 | 38.136 | 73.512 | 1.00 | 28.31 | A | C |
| ATOM | 2375 | C | PRO | A | 331 | 40.391 | 41.338 | 73.077 | 1.00 | 27.44 | A | C |
| ATOM | 2376 | O | PRO | A | 331 | 40.293 | 42.509 | 73.435 | 1.00 | 26.26 | A | O |
| ATOM | 2377 | N | ILE | A | 332 | 41.557 | 40.773 | 72.777 | 1.00 | 27.08 | A | N |
| ATOM | 2378 | CA | ILE | A | 332 | 42.805 | 41.513 | 72.882 | 1.00 | 25.78 | A | C |
| ATOM | 2379 | CB | ILE | A | 332 | 44.024 | 40.556 | 72.843 | 1.00 | 27.50 | A | C |
| ATOM | 2380 | CG2 | ILE | A | 332 | 45.295 | 41.320 | 72.500 | 1.00 | 23.92 | A | C |
| ATOM | 2381 | CG1 | ILE | A | 332 | 44.152 | 39.853 | 74.202 | 1.00 | 26.43 | A | C |
| ATOM | 2382 | CD1 | ILE | A | 332 | 45.320 | 38.905 | 74.310 | 1.00 | 26.77 | A | C |
| ATOM | 2383 | C | ILE | A | 332 | 42.904 | 42.577 | 71.791 | 1.00 | 27.70 | A | C |
| ATOM | 2384 | O | ILE | A | 332 | 43.403 | 43.674 | 72.045 | 1.00 | 28.08 | A | O |
| ATOM | 2385 | N | SER | A | 333 | 42.433 | 42.269 | 70.585 | 1.00 | 25.90 | A | N |
| ATOM | 2386 | CA | SER | A | 333 | 42.441 | 43.265 | 69.516 | 1.00 | 24.35 | A | C |
| ATOM | 2387 | CB | SER | A | 333 | 41.883 | 42.674 | 68.219 | 1.00 | 24.95 | A | C |
| ATOM | 2388 | OG | SER | A | 333 | 42.788 | 41.749 | 67.653 | 1.00 | 27.06 | A | O |
| ATOM | 2389 | C | SER | A | 333 | 41.565 | 44.454 | 69.949 | 1.00 | 25.77 | A | C |
| ATOM | 2390 | O | SER | A | 333 | 41.884 | 45.607 | 69.679 | 1.00 | 24.66 | A | O |
| ATOM | 2391 | N | ARG | A | 334 | 40.450 | 44.170 | 70.615 | 1.00 | 25.55 | A | N |
| ATOM | 2392 | CA | ARG | A | 334 | 39.575 | 45.241 | 71.068 | 1.00 | 25.27 | A | C |
| ATOM | 2393 | CB | ARG | A | 334 | 38.302 | 44.680 | 71.687 | 1.00 | 25.71 | A | C |
| ATOM | 2394 | CG | ARG | A | 334 | 37.354 | 45.750 | 72.166 | 1.00 | 27.66 | A | C |
| ATOM | 2395 | CD | ARG | A | 334 | 36.509 | 45.217 | 73.296 | 1.00 | 31.98 | A | C |
| ATOM | 2396 | NE | ARG | A | 334 | 35.184 | 44.792 | 72.871 | 1.00 | 35.51 | A | N |
| ATOM | 2397 | CZ | ARG | A | 334 | 34.425 | 43.937 | 73.549 | 1.00 | 35.07 | A | C |
| ATOM | 2398 | NH1 | ARG | A | 334 | 34.864 | 43.399 | 74.689 | 1.00 | 33.05 | A | N |
| ATOM | 2399 | NH2 | ARG | A | 334 | 33.217 | 43.641 | 73.091 | 1.00 | 35.56 | A | N |
| ATOM | 2400 | C | ARG | A | 334 | 40.289 | 46.139 | 72.077 | 1.00 | 27.54 | A | C |
| ATOM | 2401 | O | ARG | A | 334 | 40.062 | 47.347 | 72.095 | 1.00 | 27.06 | A | O |
| ATOM | 2402 | N | LEU | A | 335 | 41.153 | 45.552 | 72.910 | 1.00 | 26.22 | A | N |
| ATOM | 2403 | CA | LEU | A | 335 | 41.911 | 46.324 | 73.894 | 1.00 | 21.57 | A | C |
| ATOM | 2404 | CB | LEU | A | 335 | 42.691 | 45.402 | 74.841 | 1.00 | 21.27 | A | C |
| ATOM | 2405 | CG | LEU | A | 335 | 41.842 | 44.525 | 75.772 | 1.00 | 21.38 | A | C |
| ATOM | 2406 | CD1 | LEU | A | 335 | 42.742 | 43.731 | 76.689 | 1.00 | 17.11 | A | C |
| ATOM | 2407 | CD2 | LEU | A | 335 | 40.908 | 45.398 | 76.593 | 1.00 | 20.37 | A | C |
| ATOM | 2408 | C | LEU | A | 335 | 42.866 | 47.265 | 73.160 | 1.00 | 22.07 | A | C |
| ATOM | 2409 | O | LEU | A | 335 | 42.976 | 48.428 | 73.525 | 1.00 | 21.63 | A | O |
| ATOM | 2410 | N | TYR | A | 336 | 43.537 | 46.781 | 72.113 | 1.00 | 22.15 | A | N |
| ATOM | 2411 | CA | TYR | A | 336 | 44.458 | 47.644 | 71.359 | 1.00 | 23.02 | A | C |
| ATOM | 2412 | CB | TYR | A | 336 | 45.129 | 46.892 | 70.193 | 1.00 | 24.43 | A | C |
| ATOM | 2413 | CG | TYR | A | 336 | 46.358 | 46.103 | 70.582 | 1.00 | 28.04 | A | C |
| ATOM | 2414 | CD1 | TYR | A | 336 | 46.301 | 44.719 | 70.753 | 1.00 | 27.43 | A | C |
| ATOM | 2415 | CE1 | TYR | A | 336 | 47.418 | 43.997 | 71.131 | 1.00 | 29.24 | A | C |
| ATOM | 2416 | CD2 | TYR | A | 336 | 47.578 | 46.746 | 70.802 | 1.00 | 29.60 | A | C |
| ATOM | 2417 | CE2 | TYR | A | 336 | 48.707 | 46.033 | 71.183 | 1.00 | 28.76 | A | C |
| ATOM | 2418 | CZ | TYR | A | 336 | 48.622 | 44.660 | 71.347 | 1.00 | 30.73 | A | C |
| ATOM | 2419 | OH | TYR | A | 336 | 49.741 | 43.958 | 71.739 | 1.00 | 30.15 | A | O |
| ATOM | 2420 | C | TYR | A | 336 | 43.711 | 48.858 | 70.808 | 1.00 | 24.84 | A | C |
| ATOM | 2421 | O | TYR | A | 336 | 44.251 | 49.969 | 70.771 | 1.00 | 23.19 | A | O |
| ATOM | 2422 | N | ALA | A | 337 | 42.467 | 48.645 | 70.391 | 1.00 | 23.34 | A | N |
| ATOM | 2423 | CA | ALA | A | 337 | 41.656 | 49.725 | 69.857 | 1.00 | 23.83 | A | C |
| ATOM | 2424 | CB | ALA | A | 337 | 40.402 | 49.154 | 69.179 | 1.00 | 22.31 | A | C |
| ATOM | 2425 | C | ALA | A | 337 | 41.260 | 50.697 | 70.972 | 1.00 | 25.14 | A | C |
| ATOM | 2426 | O | ALA | A | 337 | 41.278 | 51.928 | 70.793 | 1.00 | 22.13 | A | O |
| ATOM | 2427 | N | LYS | A | 338 | 40.921 | 50.137 | 72.129 | 1.00 | 26.11 | A | N |
| ATOM | 2428 | CA | LYS | A | 338 | 40.492 | 50.960 | 73.246 | 1.00 | 26.41 | A | C |
| ATOM | 2429 | CB | LYS | A | 338 | 39.853 | 50.110 | 74.359 | 1.00 | 27.36 | A | C |
| ATOM | 2430 | CG | LYS | A | 338 | 38.422 | 49.646 | 74.067 | 1.00 | 29.20 | A | C |
| ATOM | 2431 | CD | LYS | A | 338 | 37.847 | 48.922 | 75.264 | 1.00 | 33.61 | A | C |
| ATOM | 2432 | CE | LYS | A | 338 | 36.472 | 48.347 | 74.973 | 1.00 | 37.01 | A | C |
| ATOM | 2433 | NZ | LYS | A | 338 | 35.428 | 49.370 | 74.703 | 1.00 | 40.68 | A | N |
| ATOM | 2434 | C | LYS | A | 338 | 41.666 | 51.725 | 73.788 | 1.00 | 26.06 | A | C |
| ATOM | 2435 | O | LYS | A | 338 | 41.510 | 52.829 | 74.271 | 1.00 | 25.74 | A | O |
| ATOM | 2436 | N | TYR | A | 339 | 42.853 | 51.151 | 73.655 | 1.00 | 25.32 | A | N |
| ATOM | 2437 | CA | TYR | A | 339 | 44.063 | 51.771 | 74.169 | 1.00 | 25.91 | A | C |
| ATOM | 2438 | CB | TYR | A | 339 | 45.279 | 50.984 | 73.666 | 1.00 | 23.49 | A | C |
| ATOM | 2439 | CG | TYR | A | 339 | 46.590 | 51.491 | 74.203 | 1.00 | 22.44 | A | C |
| ATOM | 2440 | CD1 | TYR | A | 339 | 46.728 | 51.836 | 75.552 | 1.00 | 23.46 | A | C |
| ATOM | 2441 | CE1 | TYR | A | 339 | 47.933 | 52.344 | 76.046 | 1.00 | 21.99 | A | C |
| ATOM | 2442 | CD2 | TYR | A | 339 | 47.690 | 51.655 | 73.363 | 1.00 | 23.61 | A | C |
| ATOM | 2443 | CE2 | TYR | A | 339 | 48.899 | 52.153 | 73.848 | 1.00 | 21.32 | A | C |
| ATOM | 2444 | CZ | TYR | A | 339 | 49.009 | 52.499 | 75.184 | 1.00 | 22.73 | A | C |
| ATOM | 2445 | OH | TYR | A | 339 | 50.195 | 53.027 | 75.626 | 1.00 | 24.19 | A | O |
| ATOM | 2446 | C | TYR | A | 339 | 44.209 | 53.276 | 73.881 | 1.00 | 26.51 | A | C |
| ATOM | 2447 | O | TYR | A | 339 | 44.609 | 54.047 | 74.756 | 1.00 | 26.37 | A | O |
| ATOM | 2448 | N | PHE | A | 340 | 43.894 | 53.693 | 72.661 | 1.00 | 27.90 | A | N |
| ATOM | 2449 | CA | PHE | A | 340 | 43.973 | 55.108 | 72.299 | 1.00 | 27.03 | A | C |
| ATOM | 2450 | CB | PHE | A | 340 | 44.852 | 55.303 | 71.050 | 1.00 | 26.85 | A | C |
| ATOM | 2451 | CG | PHE | A | 340 | 46.331 | 55.378 | 71.355 | 1.00 | 27.39 | A | C |
| ATOM | 2452 | CD1 | PHE | A | 340 | 46.851 | 56.448 | 72.089 | 1.00 | 26.18 | A | C |
| ATOM | 2453 | CD2 | PHE | A | 340 | 47.192 | 54.351 | 70.971 | 1.00 | 26.30 | A | C |
| ATOM | 2454 | CE1 | PHE | A | 340 | 48.209 | 56.487 | 72.446 | 1.00 | 25.70 | A | C |
| ATOM | 2455 | CE2 | PHE | A | 340 | 48.551 | 54.384 | 71.323 | 1.00 | 25.57 | A | C |
| ATOM | 2456 | CZ | PHE | A | 340 | 49.058 | 55.452 | 72.061 | 1.00 | 24.07 | A | C |
| ATOM | 2457 | C | PHE | A | 340 | 42.581 | 55.710 | 72.070 | 1.00 | 27.01 | A | C |
| ATOM | 2458 | O | PHE | A | 340 | 42.382 | 56.481 | 71.126 | 1.00 | 26.84 | A | O |
| ATOM | 2459 | N | GLN | A | 341 | 41.625 | 55.337 | 72.928 | 1.00 | 24.47 | A | N |
| ATOM | 2460 | CA | GLN | A | 341 | 40.248 | 55.846 | 72.872 | 1.00 | 24.84 | A | C |
| ATOM | 2461 | CB | GLN | A | 341 | 40.242 | 57.383 | 72.900 | 1.00 | 22.81 | A | C |
| ATOM | 2462 | CG | GLN | A | 341 | 41.069 | 58.035 | 74.008 | 1.00 | 18.92 | A | C |
| ATOM | 2463 | CD | GLN | A | 341 | 40.607 | 57.677 | 75.406 | 1.00 | 22.62 | A | C |
| ATOM | 2464 | OE1 | GLN | A | 341 | 39.410 | 57.599 | 75.681 | 1.00 | 24.85 | A | O |
| ATOM | 2465 | NE2 | GLN | A | 341 | 41.563 | 57.483 | 76.309 | 1.00 | 20.95 | A | N |
| ATOM | 2466 | C | GLN | A | 341 | 39.425 | 55.373 | 71.673 | 1.00 | 25.53 | A | C |
| ATOM | 2467 | O | GLN | A | 341 | 38.496 | 56.061 | 71.232 | 1.00 | 28.47 | A | O |
| ATOM | 2468 | N | GLY | A | 342 | 39.761 | 54.205 | 71.141 | 1.00 | 25.93 | A | N |
| ATOM | 2469 | CA | GLY | A | 342 | 39.015 | 53.679 | 70.014 | 1.00 | 22.80 | A | C |
| ATOM | 2470 | C | GLY | A | 342 | 38.190 | 52.525 | 70.525 | 1.00 | 23.52 | A | C |
| ATOM | 2471 | O | GLY | A | 342 | 38.073 | 52.354 | 71.734 | 1.00 | 23.53 | A | O |
| ATOM | 2472 | N | ASP | A | 343 | 37.619 | 51.737 | 69.621 | 1.00 | 25.24 | A | N |
| ATOM | 2473 | CA | ASP | A | 343 | 36.815 | 50.581 | 70.006 | 1.00 | 23.28 | A | C |
| ATOM | 2474 | CB | ASP | A | 343 | 35.372 | 51.014 | 70.307 | 1.00 | 23.63 | A | C |
| ATOM | 2475 | CG | ASP | A | 343 | 34.598 | 49.973 | 71.120 | 1.00 | 26.01 | A | C |
| ATOM | 2476 | OD1 | ASP | A | 343 | 35.119 | 48.859 | 71.340 | 1.00 | 29.66 | A | O |
| ATOM | 2477 | OD2 | ASP | A | 343 | 33.463 | 50.265 | 71.543 | 1.00 | 27.56 | A | O |
| ATOM | 2478 | C | ASP | A | 343 | 36.843 | 49.605 | 68.824 | 1.00 | 21.97 | A | C |
| ATOM | 2479 | O | ASP | A | 343 | 37.371 | 49.926 | 67.756 | 1.00 | 23.24 | A | O |
| ATOM | 2480 | N | LEU | A | 344 | 36.301 | 48.413 | 69.022 | 1.00 | 19.85 | A | N |
| ATOM | 2481 | CA | LEU | A | 344 | 36.241 | 47.412 | 67.965 | 1.00 | 18.89 | A | C |
| ATOM | 2482 | CB | LEU | A | 344 | 37.445 | 46.476 | 68.060 | 1.00 | 17.82 | A | C |
| ATOM | 2483 | CG | LEU | A | 344 | 37.655 | 45.496 | 66.905 | 1.00 | 21.08 | A | C |
| ATOM | 2484 | CD1 | LEU | A | 344 | 39.059 | 44.917 | 67.007 | 1.00 | 22.30 | A | C |
| ATOM | 2485 | CD2 | LEU | A | 344 | 36.585 | 44.387 | 66.925 | 1.00 | 18.17 | A | C |
| ATOM | 2486 | C | LEU | A | 344 | 34.936 | 46.668 | 68.224 | 1.00 | 19.54 | A | C |
| ATOM | 2487 | O | LEU | A | 344 | 34.746 | 46.114 | 69.301 | 1.00 | 18.73 | A | O |
| ATOM | 2488 | N | GLN | A | 345 | 34.033 | 46.681 | 67.249 | 1.00 | 20.91 | A | N |
| ATOM | 2489 | CA | GLN | A | 345 | 32.733 | 46.038 | 67.413 | 1.00 | 23.28 | A | C |
| ATOM | 2490 | CB | GLN | A | 345 | 31.632 | 47.112 | 67.502 | 1.00 | 22.21 | A | C |
| ATOM | 2491 | CG | GLN | A | 345 | 31.907 | 48.172 | 68.556 | 1.00 | 24.85 | A | C |
| ATOM | 2492 | CD | GLN | A | 345 | 30.845 | 49.262 | 68.627 | 1.00 | 28.08 | A | C |
| ATOM | 2493 | OE1 | GLN | A | 345 | 29.950 | 49.346 | 67.788 | 1.00 | 29.13 | A | O |
| ATOM | 2494 | NE2 | GLN | A | 345 | 30.956 | 50.116 | 69.638 | 1.00 | 29.92 | A | N |
| ATOM | 2495 | C | GLN | A | 345 | 32.398 | 45.063 | 66.290 | 1.00 | 25.13 | A | C |
| ATOM | 2496 | O | GLN | A | 345 | 32.925 | 45.162 | 65.176 | 1.00 | 25.38 | A | O |
| ATOM | 2497 | N | LEU | A | 346 | 31.524 | 44.112 | 66.601 | 1.00 | 24.07 | A | N |
| ATOM | 2498 | CA | LEU | A | 346 | 31.084 | 43.123 | 65.632 | 1.00 | 24.31 | A | C |
| ATOM | 2499 | CB | LEU | A | 346 | 31.519 | 41.717 | 66.028 | 1.00 | 25.93 | A | C |
| ATOM | 2500 | CG | LEU | A | 346 | 32.977 | 41.290 | 65.932 | 1.00 | 31.28 | A | C |
| ATOM | 2501 | CD1 | LEU | A | 346 | 33.024 | 39.786 | 66.158 | 1.00 | 30.88 | A | C |
| ATOM | 2502 | CD2 | LEU | A | 346 | 33.564 | 41.628 | 64.565 | 1.00 | 31.06 | A | C |
| ATOM | 2503 | C | LEU | A | 346 | 29.577 | 43.085 | 65.540 | 1.00 | 24.63 | A | C |
| ATOM | 2504 | O | LEU | A | 346 | 28.882 | 43.266 | 66.533 | 1.00 | 26.22 | A | O |
| ATOM | 2505 | N | PHE | A | 347 | 29.077 | 42.852 | 64.339 | 1.00 | 23.36 | A | N |
| ATOM | 2506 | CA | PHE | A | 347 | 27.648 | 42.677 | 64.126 | 1.00 | 24.55 | A | C |
| ATOM | 2507 | CB | PHE | A | 347 | 26.891 | 44.008 | 64.079 | 1.00 | 28.19 | A | C |
| ATOM | 2508 | CG | PHE | A | 347 | 27.743 | 45.169 | 63.750 | 1.00 | 32.00 | A | C |
| ATOM | 2509 | CD1 | PHE | A | 347 | 27.968 | 45.536 | 62.427 | 1.00 | 39.02 | A | C |
| ATOM | 2510 | CD2 | PHE | A | 347 | 28.364 | 45.879 | 64.766 | 1.00 | 37.16 | A | C |
| ATOM | 2511 | CE1 | PHE | A | 347 | 28.814 | 46.607 | 62.120 | 1.00 | 40.22 | A | C |
| ATOM | 2512 | CE2 | PHE | A | 347 | 29.210 | 46.946 | 64.484 | 1.00 | 38.67 | A | C |
| ATOM | 2513 | CZ | PHE | A | 347 | 29.437 | 47.311 | 63.155 | 1.00 | 41.13 | A | C |
| ATOM | 2514 | C | PHE | A | 347 | 27.479 | 41.914 | 62.841 | 1.00 | 22.81 | A | C |
| ATOM | 2515 | O | PHE | A | 347 | 27.972 | 42.325 | 61.793 | 1.00 | 21.28 | A | O |
| ATOM | 2516 | N | SER | A | 348 | 26.813 | 40.776 | 62.923 | 1.00 | 22.19 | A | N |
| ATOM | 2517 | CA | SER | A | 348 | 26.613 | 39.990 | 61.726 | 1.00 | 26.43 | A | C |
| ATOM | 2518 | CB | SER | A | 348 | 27.282 | 38.614 | 61.839 | 1.00 | 26.04 | A | C |
| ATOM | 2519 | OG | SER | A | 348 | 26.762 | 37.879 | 62.916 | 1.00 | 27.21 | A | O |
| ATOM | 2520 | C | SER | A | 348 | 25.148 | 39.807 | 61.488 | 1.00 | 27.10 | A | C |
| ATOM | 2521 | O | SER | A | 348 | 24.314 | 40.279 | 62.256 | 1.00 | 25.76 | A | O |
| ATOM | 2522 | N | MET | A | 349 | 24.863 | 39.100 | 60.405 | 1.00 | 29.17 | A | N |
| ATOM | 2523 | CA | MET | A | 349 | 23.516 | 38.788 | 59.969 | 1.00 | 28.98 | A | C |
| ATOM | 2524 | CB | MET | A | 349 | 23.254 | 39.445 | 58.621 | 1.00 | 30.87 | A | C |
| ATOM | 2525 | CG | MET | A | 349 | 23.592 | 40.919 | 58.554 | 1.00 | 35.19 | A | C |
| ATOM | 2526 | SD | MET | A | 349 | 22.313 | 41.905 | 59.334 | 1.00 | 41.79 | A | S |
| ATOM | 2527 | CE | MET | A | 349 | 21.023 | 41.857 | 58.082 | 1.00 | 38.70 | A | C |
| ATOM | 2528 | C | MET | A | 349 | 23.571 | 37.286 | 59.782 | 1.00 | 27.29 | A | C |
| ATOM | 2529 | O | MET | A | 349 | 24.161 | 36.817 | 58.816 | 1.00 | 27.82 | A | O |
| ATOM | 2530 | N | GLU | A | 350 | 22.980 | 36.524 | 60.691 | 1.00 | 25.00 | A | N |
| ATOM | 2531 | CA | GLU | A | 350 | 23.042 | 35.083 | 60.536 | 1.00 | 26.98 | A | C |
| ATOM | 2532 | CB | GLU | A | 350 | 22.270 | 34.370 | 61.640 | 1.00 | 27.32 | A | C |
| ATOM | 2533 | CG | GLU | A | 350 | 22.648 | 32.911 | 61.753 | 1.00 | 27.54 | A | C |
| ATOM | 2534 | CD | GLU | A | 350 | 21.658 | 32.095 | 62.561 | 1.00 | 29.25 | A | C |
| ATOM | 2535 | OE1 | GLU | A | 350 | 22.039 | 30.980 | 62.973 | 1.00 | 31.35 | A | O |
| ATOM | 2536 | OE2 | GLU | A | 350 | 20.509 | 32.549 | 62.773 | 1.00 | 29.04 | A | O |
| ATOM | 2537 | C | GLU | A | 350 | 22.449 | 34.716 | 59.186 | 1.00 | 25.67 | A | C |
| ATOM | 2538 | O | GLU | A | 350 | 21.370 | 35.187 | 58.840 | 1.00 | 26.07 | A | O |
| ATOM | 2539 | N | GLY | A | 351 | 23.165 | 33.895 | 58.424 | 1.00 | 23.64 | A | N |
| ATOM | 2540 | CA | GLY | A | 351 | 22.684 | 33.475 | 57.120 | 1.00 | 24.71 | A | C |
| ATOM | 2541 | C | GLY | A | 351 | 23.289 | 34.301 | 56.004 | 1.00 | 26.57 | A | C |
| ATOM | 2542 | O | GLY | A | 351 | 23.069 | 34.033 | 54.822 | 1.00 | 27.99 | A | O |
| ATOM | 2543 | N | PHE | A | 352 | 24.070 | 35.309 | 56.369 | 1.00 | 26.58 | A | N |
| ATOM | 2544 | CA | PHE | A | 352 | 24.670 | 36.160 | 55.358 | 1.00 | 27.37 | A | C |
| ATOM | 2545 | CB | PHE | A | 352 | 23.896 | 37.475 | 55.260 | 1.00 | 26.58 | A | C |
| ATOM | 2546 | CG | PHE | A | 352 | 24.422 | 38.399 | 54.204 | 1.00 | 28.09 | A | C |
| ATOM | 2547 | CD1 | PHE | A | 352 | 24.240 | 38.104 | 52.857 | 1.00 | 30.16 | A | C |
| ATOM | 2548 | CD2 | PHE | A | 352 | 25.150 | 39.531 | 54.544 | 1.00 | 28.35 | A | C |
| ATOM | 2549 | CE1 | PHE | A | 352 | 24.774 | 38.921 | 51.861 | 1.00 | 29.05 | A | C |
| ATOM | 2550 | CE2 | PHE | A | 352 | 25.693 | 40.360 | 53.546 | 1.00 | 30.26 | A | C |
| ATOM | 2551 | CZ | PHE | A | 352 | 25.503 | 40.048 | 52.204 | 1.00 | 28.14 | A | C |
| ATOM | 2552 | C | PHE | A | 352 | 26.160 | 36.466 | 55.562 | 1.00 | 28.88 | A | C |
| ATOM | 2553 | O | PHE | A | 352 | 27.021 | 35.949 | 54.838 | 1.00 | 29.61 | A | O |
| ATOM | 2554 | N | GLY | A | 353 | 26.460 | 37.325 | 56.531 | 1.00 | 28.37 | A | N |
| ATOM | 2555 | CA | GLY | A | 353 | 27.843 | 37.689 | 56.782 | 1.00 | 28.31 | A | C |
| ATOM | 2556 | C | GLY | A | 353 | 28.056 | 38.503 | 58.043 | 1.00 | 27.87 | A | C |
| ATOM | 2557 | O | GLY | A | 353 | 27.137 | 38.685 | 58.832 | 1.00 | 28.13 | A | O |
| ATOM | 2558 | N | THR | A | 354 | 29.270 | 39.008 | 58.230 | 1.00 | 27.99 | A | N |
| ATOM | 2559 | CA | THR | A | 354 | 29.577 | 39.784 | 59.417 | 1.00 | 24.83 | A | C |
| ATOM | 2560 | CB | THR | A | 354 | 30.421 | 38.967 | 60.407 | 1.00 | 24.46 | A | C |
| ATOM | 2561 | OG1 | THR | A | 354 | 29.720 | 37.775 | 60.787 | 1.00 | 23.27 | A | O |
| ATOM | 2562 | CG2 | THR | A | 354 | 30.731 | 39.806 | 61.638 | 1.00 | 18.98 | A | C |
| ATOM | 2563 | C | THR | A | 354 | 30.333 | 41.074 | 59.117 | 1.00 | 27.63 | A | C |
| ATOM | 2564 | O | THR | A | 354 | 31.187 | 41.125 | 58.223 | 1.00 | 28.06 | A | O |
| ATOM | 2565 | N | ASP | A | 355 | 29.989 | 42.120 | 59.862 | 1.00 | 25.90 | A | N |
| ATOM | 2566 | CA | ASP | A | 355 | 30.658 | 43.405 | 59.736 | 1.00 | 25.98 | A | C |
| ATOM | 2567 | CB | ASP | A | 355 | 29.659 | 44.551 | 59.630 | 1.00 | 25.04 | A | C |
| ATOM | 2568 | CG | ASP | A | 355 | 29.224 | 44.826 | 58.209 | 1.00 | 26.88 | A | C |
| ATOM | 2569 | OD1 | ASP | A | 355 | 29.510 | 44.013 | 57.297 | 1.00 | 24.25 | A | O |
| ATOM | 2570 | OD2 | ASP | A | 355 | 28.576 | 45.874 | 58.017 | 1.00 | 26.10 | A | O |
| ATOM | 2571 | C | ASP | A | 355 | 31.474 | 43.600 | 60.997 | 1.00 | 27.04 | A | C |
| ATOM | 2572 | O | ASP | A | 355 | 30.962 | 43.439 | 62.110 | 1.00 | 27.15 | A | O |
| ATOM | 2573 | N | ALA | A | 356 | 32.754 | 43.908 | 60.823 | 1.00 | 25.33 | A | N |
| ATOM | 2574 | CA | ALA | A | 356 | 33.621 | 44.168 | 61.957 | 1.00 | 23.06 | A | C |
| ATOM | 2575 | CB | ALA | A | 356 | 34.786 | 43.192 | 61.990 | 1.00 | 24.45 | A | C |
| ATOM | 2576 | C | ALA | A | 356 | 34.119 | 45.578 | 61.729 | 1.00 | 24.05 | A | C |
| ATOM | 2577 | O | ALA | A | 356 | 34.555 | 45.915 | 60.637 | 1.00 | 23.01 | A | O |
| ATOM | 2578 | N | VAL | A | 357 | 34.047 | 46.404 | 62.760 | 1.00 | 23.41 | A | N |
| ATOM | 2579 | CA | VAL | A | 357 | 34.468 | 47.780 | 62.639 | 1.00 | 22.44 | A | C |
| ATOM | 2580 | CB | VAL | A | 357 | 33.262 | 48.734 | 62.770 | 1.00 | 25.41 | A | C |
| ATOM | 2581 | CG1 | VAL | A | 357 | 33.712 | 50.182 | 62.572 | 1.00 | 27.66 | A | C |
| ATOM | 2582 | CG2 | VAL | A | 357 | 32.200 | 48.373 | 61.751 | 1.00 | 26.31 | A | C |
| ATOM | 2583 | C | VAL | A | 357 | 35.485 | 48.184 | 63.686 | 1.00 | 23.80 | A | C |
| ATOM | 2584 | O | VAL | A | 357 | 35.310 | 47.911 | 64.870 | 1.00 | 26.25 | A | O |
| ATOM | 2585 | N | ILE | A | 358 | 36.550 | 48.842 | 63.243 | 1.00 | 23.45 | A | N |
| ATOM | 2586 | CA | ILE | A | 358 | 37.567 | 49.341 | 64.149 | 1.00 | 23.49 | A | C |
| ATOM | 2587 | CB | ILE | A | 358 | 38.990 | 49.070 | 63.647 | 1.00 | 23.30 | A | C |
| ATOM | 2588 | CG2 | ILE | A | 358 | 39.980 | 49.696 | 64.600 | 1.00 | 22.42 | A | C |
| ATOM | 2589 | CG1 | ILE | A | 358 | 39.256 | 47.571 | 63.552 | 1.00 | 21.79 | A | C |
| ATOM | 2590 | CD1 | ILE | A | 358 | 40.598 | 47.245 | 62.927 | 1.00 | 20.36 | A | C |
| ATOM | 2591 | C | ILE | A | 358 | 37.390 | 50.848 | 64.178 | 1.00 | 21.96 | A | C |
| ATOM | 2592 | O | ILE | A | 358 | 37.474 | 51.495 | 63.140 | 1.00 | 24.25 | A | O |
| ATOM | 2593 | N | TYR | A | 359 | 37.125 | 51.397 | 65.358 | 1.00 | 20.31 | A | N |
| ATOM | 2594 | CA | TYR | A | 359 | 36.959 | 52.833 | 65.525 | 1.00 | 20.60 | A | C |
| ATOM | 2595 | CB | TYR | A | 359 | 35.753 | 53.155 | 66.423 | 1.00 | 20.99 | A | C |
| ATOM | 2596 | CG | TYR | A | 359 | 34.399 | 52.800 | 65.839 | 1.00 | 20.07 | A | C |
| ATOM | 2597 | CD1 | TYR | A | 359 | 33.724 | 51.655 | 66.253 | 1.00 | 20.50 | A | C |
| ATOM | 2598 | CE1 | TYR | A | 359 | 32.486 | 51.306 | 65.706 | 1.00 | 24.15 | A | C |
| ATOM | 2599 | CD2 | TYR | A | 359 | 33.804 | 53.601 | 64.853 | 1.00 | 20.29 | A | C |
| ATOM | 2600 | CE2 | TYR | A | 359 | 32.564 | 53.255 | 64.293 | 1.00 | 22.17 | A | C |
| ATOM | 2601 | CZ | TYR | A | 359 | 31.918 | 52.107 | 64.731 | 1.00 | 22.17 | A | C |
| ATOM | 2602 | OH | TYR | A | 359 | 30.704 | 51.746 | 64.215 | 1.00 | 25.77 | A | O |
| ATOM | 2603 | C | TYR | A | 359 | 38.212 | 53.427 | 66.160 | 1.00 | 20.62 | A | C |
| ATOM | 2604 | O | TYR | A | 359 | 38.603 | 53.040 | 67.261 | 1.00 | 21.35 | A | O |
| ATOM | 2605 | N | LEU | A | 360 | 38.845 | 54.359 | 65.459 | 1.00 | 20.90 | A | N |
| ATOM | 2606 | CA | LEU | A | 360 | 40.035 | 55.037 | 65.967 | 1.00 | 22.03 | A | C |
| ATOM | 2607 | CB | LEU | A | 360 | 41.225 | 54.816 | 65.027 | 1.00 | 21.07 | A | C |
| ATOM | 2608 | CG | LEU | A | 360 | 41.614 | 53.379 | 64.681 | 1.00 | 21.75 | A | C |
| ATOM | 2609 | CD1 | LEU | A | 360 | 42.676 | 53.397 | 63.582 | 1.00 | 16.88 | A | C |
| ATOM | 2610 | CD2 | LEU | A | 360 | 42.126 | 52.661 | 65.953 | 1.00 | 18.45 | A | C |
| ATOM | 2611 | C | LEU | A | 360 | 39.740 | 56.538 | 66.016 | 1.00 | 23.54 | A | C |
| ATOM | 2612 | O | LEU | A | 360 | 38.833 | 57.011 | 65.333 | 1.00 | 21.36 | A | O |
| ATOM | 2613 | N | LYS | A | 361 | 40.484 | 57.279 | 66.836 | 1.00 | 23.81 | A | N |
| ATOM | 2614 | CA | LYS | A | 361 | 40.313 | 58.729 | 66.879 | 1.00 | 25.86 | A | C |
| ATOM | 2615 | CB | LYS | A | 361 | 40.900 | 59.342 | 68.150 | 1.00 | 26.27 | A | C |
| ATOM | 2616 | CG | LYS | A | 361 | 40.149 | 59.048 | 69.417 | 1.00 | 30.66 | A | C |
| ATOM | 2617 | CD | LYS | A | 361 | 38.724 | 59.575 | 69.373 | 1.00 | 29.23 | A | C |
| ATOM | 2618 | CE | LYS | A | 361 | 38.107 | 59.529 | 70.775 | 1.00 | 30.65 | A | C |
| ATOM | 2619 | NZ | LYS | A | 361 | 36.638 | 59.764 | 70.764 | 1.00 | 30.51 | A | N |
| ATOM | 2620 | C | LYS | A | 361 | 41.097 | 59.278 | 65.685 | 1.00 | 28.34 | A | C |
| ATOM | 2621 | O | LYS | A | 361 | 42.271 | 58.942 | 65.501 | 1.00 | 28.87 | A | O |
| ATOM | 2622 | N | ALA | A | 362 | 40.456 | 60.117 | 64.878 | 1.00 | 28.59 | A | N |
| ATOM | 2623 | CA | ALA | A | 362 | 41.113 | 60.704 | 63.714 | 1.00 | 28.93 | A | C |
| ATOM | 2624 | CB | ALA | A | 362 | 40.070 | 61.403 | 62.826 | 1.00 | 29.84 | A | C |
| ATOM | 2625 | C | ALA | A | 362 | 42.220 | 61.698 | 64.096 | 1.00 | 29.95 | A | C |
| ATOM | 2626 | O | ALA | A | 362 | 43.247 | 61.776 | 63.429 | 1.00 | 29.64 | A | O |
| ATOM | 2627 | N | LEU | A | 363 | 42.017 | 62.441 | 65.182 | 1.00 | 30.30 | A | N |
| ATOM | 2628 | CA | LEU | A | 363 | 42.980 | 63.457 | 65.605 | 1.00 | 31.10 | A | C |
| ATOM | 2629 | CB | LEU | A | 363 | 42.214 | 64.698 | 66.085 | 1.00 | 29.62 | A | C |
| ATOM | 2630 | CG | LEU | A | 363 | 41.124 | 65.236 | 65.144 | 1.00 | 33.90 | A | C |
| ATOM | 2631 | CD1 | LEU | A | 363 | 40.464 | 66.481 | 65.766 | 1.00 | 30.58 | A | C |
| ATOM | 2632 | CD2 | LEU | A | 363 | 41.737 | 65.557 | 63.791 | 1.00 | 30.29 | A | C |
| ATOM | 2633 | C | LEU | A | 363 | 43.990 | 63.022 | 66.679 | 1.00 | 32.09 | A | C |
| ATOM | 2634 | O | LEU | A | 363 | 43.646 | 62.327 | 67.635 | 1.00 | 29.76 | A | O |
| ATOM | 2635 | N | SER | A | 364 | 45.239 | 63.453 | 66.529 | 1.00 | 31.94 | A | N |
| ATOM | 2636 | CA | SER | A | 364 | 46.267 | 63.091 | 67.498 | 1.00 | 33.82 | A | C |
| ATOM | 2637 | CB | SER | A | 364 | 47.654 | 63.514 | 67.001 | 1.00 | 32.60 | A | C |
| ATOM | 2638 | OG | SER | A | 364 | 47.758 | 64.919 | 66.865 | 1.00 | 36.25 | A | O |
| ATOM | 2639 | C | SER | A | 364 | 45.972 | 63.713 | 68.865 | 1.00 | 35.21 | A | C |
| ATOM | 2640 | O | SER | A | 364 | 46.411 | 63.205 | 69.898 | 1.00 | 34.98 | A | O |
| ATOM | 2641 | N | THR | A | 365 | 45.211 | 64.801 | 68.874 | 1.00 | 36.20 | A | N |
| ATOM | 2642 | CA | THR | A | 365 | 44.868 | 65.452 | 70.133 | 1.00 | 37.09 | A | C |
| ATOM | 2643 | CB | THR | A | 365 | 44.269 | 66.867 | 69.900 | 1.00 | 38.80 | A | C |
| ATOM | 2644 | OG1 | THR | A | 365 | 42.973 | 66.761 | 69.295 | 1.00 | 43.79 | A | O |
| ATOM | 2645 | CG2 | THR | A | 365 | 45.155 | 67.665 | 68.972 | 1.00 | 39.90 | A | C |
| ATOM | 2646 | C | THR | A | 365 | 43.871 | 64.609 | 70.936 | 1.00 | 36.15 | A | C |
| ATOM | 2647 | O | THR | A | 365 | 43.845 | 64.692 | 72.159 | 1.00 | 37.69 | A | O |
| ATOM | 2648 | N | ASP | A | 366 | 43.061 | 63.796 | 70.256 | 1.00 | 35.63 | A | N |
| ATOM | 2649 | CA | ASP | A | 366 | 42.069 | 62.958 | 70.935 | 1.00 | 34.85 | A | C |
| ATOM | 2650 | CB | ASP | A | 366 | 40.800 | 62.796 | 70.079 | 1.00 | 35.59 | A | C |
| ATOM | 2651 | CG | ASP | A | 366 | 40.055 | 64.109 | 69.858 | 1.00 | 36.39 | A | C |
| ATOM | 2652 | OD1 | ASP | A | 366 | 39.901 | 64.885 | 70.817 | 1.00 | 40.61 | A | O |
| ATOM | 2653 | OD2 | ASP | A | 366 | 39.599 | 64.360 | 68.723 | 1.00 | 38.62 | A | O |
| ATOM | 2654 | C | ASP | A | 366 | 42.631 | 61.570 | 71.245 | 1.00 | 34.24 | A | C |
| ATOM | 2655 | O | ASP | A | 366 | 41.962 | 60.740 | 71.869 | 1.00 | 34.65 | A | O |
| ATOM | 2656 | N | SER | A | 367 | 43.857 | 61.318 | 70.802 | 1.00 | 33.22 | A | N |
| ATOM | 2657 | CA | SER | A | 367 | 44.482 | 60.024 | 71.034 | 1.00 | 33.11 | A | C |
| ATOM | 2658 | CB | SER | A | 367 | 45.385 | 59.643 | 69.863 | 1.00 | 33.38 | A | C |
| ATOM | 2659 | OG | SER | A | 367 | 44.643 | 59.608 | 68.658 | 1.00 | 34.88 | A | O |
| ATOM | 2660 | C | SER | A | 367 | 45.289 | 60.034 | 72.320 | 1.00 | 33.46 | A | C |
| ATOM | 2661 | O | SER | A | 367 | 46.467 | 60.389 | 72.340 | 1.00 | 33.83 | A | O |
| ATOM | 2662 | N | VAL | A | 368 | 44.643 | 59.630 | 73.400 | 1.00 | 32.22 | A | N |
| ATOM | 2663 | CA | VAL | A | 368 | 45.295 | 59.597 | 74.694 | 1.00 | 30.54 | A | C |
| ATOM | 2664 | CB | VAL | A | 368 | 44.662 | 60.637 | 75.657 | 1.00 | 29.45 | A | C |
| ATOM | 2665 | CG1 | VAL | A | 368 | 45.401 | 60.642 | 76.985 | 1.00 | 29.12 | A | C |
| ATOM | 2666 | CG2 | VAL | A | 368 | 44.686 | 62.019 | 75.034 | 1.00 | 28.99 | A | C |
| ATOM | 2667 | C | VAL | A | 368 | 45.135 | 58.208 | 75.298 | 1.00 | 31.04 | A | C |
| ATOM | 2668 | O | VAL | A | 368 | 44.053 | 57.617 | 75.237 | 1.00 | 30.67 | A | O |
| ATOM | 2669 | N | GLU | A | 369 | 46.217 | 57.704 | 75.881 | 1.00 | 29.02 | A | N |
| ATOM | 2670 | CA | GLU | A | 369 | 46.202 | 56.397 | 76.513 | 1.00 | 28.09 | A | C |
| ATOM | 2671 | CB | GLU | A | 369 | 47.439 | 56.215 | 77.400 | 1.00 | 25.60 | A | C |
| ATOM | 2672 | CG | GLU | A | 369 | 48.755 | 56.164 | 76.693 | 1.00 | 23.11 | A | C |
| ATOM | 2673 | CD | GLU | A | 369 | 49.852 | 55.790 | 77.670 | 1.00 | 27.00 | A | C |
| ATOM | 2674 | OE1 | GLU | A | 369 | 50.118 | 56.588 | 78.599 | 1.00 | 24.39 | A | O |
| ATOM | 2675 | OE2 | GLU | A | 369 | 50.434 | 54.691 | 77.533 | 1.00 | 24.32 | A | O |
| ATOM | 2676 | C | GLU | A | 369 | 44.967 | 56.202 | 77.404 | 1.00 | 28.34 | A | C |
| ATOM | 2677 | O | GLU | A | 369 | 44.563 | 57.120 | 78.126 | 1.00 | 29.02 | A | O |
| ATOM | 2678 | N | ARG | A | 370 | 44.367 | 55.014 | 77.332 | 1.00 | 27.19 | A | N |
| ATOM | 2679 | CA | ARG | A | 370 | 43.246 | 54.648 | 78.200 | 1.00 | 29.97 | A | C |
| ATOM | 2680 | CB | ARG | A | 370 | 42.122 | 53.964 | 77.404 | 1.00 | 32.39 | A | C |
| ATOM | 2681 | CG | ARG | A | 370 | 40.851 | 53.693 | 78.240 | 1.00 | 38.17 | A | C |
| ATOM | 2682 | CD | ARG | A | 370 | 39.676 | 53.242 | 77.356 | 1.00 | 43.56 | A | C |
| ATOM | 2683 | NE | ARG | A | 370 | 39.031 | 54.342 | 76.631 | 1.00 | 52.79 | A | N |
| ATOM | 2684 | CZ | ARG | A | 370 | 38.130 | 54.150 | 75.660 | 1.00 | 57.96 | A | C |
| ATOM | 2685 | NH1 | ARG | A | 370 | 37.783 | 52.907 | 75.294 | 1.00 | 60.97 | A | N |
| ATOM | 2686 | NH2 | ARG | A | 370 | 37.522 | 55.181 | 75.075 | 1.00 | 60.12 | A | N |
| ATOM | 2687 | C | ARG | A | 370 | 43.915 | 53.664 | 79.173 | 1.00 | 32.22 | A | C |
| ATOM | 2688 | O | ARG | A | 370 | 44.353 | 52.606 | 78.750 | 1.00 | 33.75 | A | O |
| ATOM | 2689 | N | LEU | A | 371 | 43.981 | 53.996 | 80.463 | 1.00 | 34.73 | A | N |
| ATOM | 2690 | CA | LEU | A | 371 | 44.672 | 53.158 | 81.444 | 1.00 | 36.28 | A | C |
| ATOM | 2691 | CB | LEU | A | 371 | 45.897 | 53.904 | 81.976 | 1.00 | 32.30 | A | C |
| ATOM | 2692 | CG | LEU | A | 371 | 46.880 | 54.498 | 80.981 | 1.00 | 33.40 | A | C |
| ATOM | 2693 | CD1 | LEU | A | 371 | 47.937 | 55.275 | 81.738 | 1.00 | 34.02 | A | C |
| ATOM | 2694 | CD2 | LEU | A | 371 | 47.515 | 53.393 | 80.157 | 1.00 | 32.06 | A | C |
| ATOM | 2695 | C | LEU | A | 371 | 43.789 | 52.821 | 82.628 | 1.00 | 40.51 | A | C |
| ATOM | 2696 | O | LEU | A | 371 | 42.978 | 53.635 | 83.058 | 1.00 | 42.15 | A | O |
| ATOM | 2697 | N | PRO | A | 372 | 43.921 | 51.606 | 83.161 | 1.00 | 44.39 | A | N |
| ATOM | 2698 | CD | PRO | A | 372 | 44.526 | 50.419 | 82.528 | 1.00 | 44.59 | A | C |
| ATOM | 2699 | CA | PRO | A | 372 | 43.104 | 51.233 | 84.310 | 1.00 | 48.53 | A | C |
| ATOM | 2700 | CB | PRO | A | 372 | 43.000 | 49.716 | 84.156 | 1.00 | 47.88 | A | C |
| ATOM | 2701 | CG | PRO | A | 372 | 44.343 | 49.352 | 83.583 | 1.00 | 45.70 | A | C |
| ATOM | 2702 | C | PRO | A | 372 | 43.813 | 51.675 | 85.592 | 1.00 | 52.04 | A | C |
| ATOM | 2703 | O | PRO | A | 372 | 45.042 | 51.750 | 85.627 | 1.00 | 52.81 | A | O |
| ATOM | 2704 | N | VAL | A | 373 | 43.047 | 51.986 | 86.633 | 1.00 | 58.32 | A | N |
| ATOM | 2705 | CA | VAL | A | 373 | 43.644 | 52.392 | 87.906 | 1.00 | 63.78 | A | C |
| ATOM | 2706 | CB | VAL | A | 373 | 43.499 | 53.902 | 88.153 | 1.00 | 63.27 | A | C |
| ATOM | 2707 | CG1 | VAL | A | 373 | 44.022 | 54.266 | 89.539 | 1.00 | 63.08 | A | C |
| ATOM | 2708 | CG2 | VAL | A | 373 | 44.272 | 54.646 | 87.109 | 1.00 | 62.29 | A | C |
| ATOM | 2709 | C | VAL | A | 373 | 43.025 | 51.675 | 89.092 | 1.00 | 68.81 | A | C |
| ATOM | 2710 | O | VAL | A | 373 | 41.828 | 51.382 | 89.095 | 1.00 | 68.56 | A | O |
| ATOM | 2711 | N | TYR | A | 374 | 43.855 | 51.404 | 90.098 | 1.00 | 75.37 | A | N |
| ATOM | 2712 | CA | TYR | A | 374 | 43.435 | 50.743 | 91.331 | 1.00 | 82.14 | A | C |
| ATOM | 2713 | CB | TYR | A | 374 | 44.664 | 50.167 | 92.053 | 1.00 | 83.39 | A | C |
| ATOM | 2714 | CG | TYR | A | 374 | 44.420 | 49.866 | 93.504 | 1.00 | 86.52 | A | C |
| ATOM | 2715 | CD1 | TYR | A | 374 | 43.456 | 48.930 | 93.891 | 1.00 | 88.22 | A | C |
| ATOM | 2716 | CE1 | TYR | A | 374 | 43.187 | 48.689 | 95.247 | 1.00 | 90.05 | A | C |
| ATOM | 2717 | CD2 | TYR | A | 374 | 45.117 | 50.551 | 94.500 | 1.00 | 87.48 | A | C |
| ATOM | 2718 | CE2 | TYR | A | 374 | 44.855 | 50.317 | 95.854 | 1.00 | 90.06 | A | C |
| ATOM | 2719 | CZ | TYR | A | 374 | 43.887 | 49.389 | 96.225 | 1.00 | 90.68 | A | C |
| ATOM | 2720 | OH | TYR | A | 374 | 43.596 | 49.183 | 97.566 | 1.00 | 92.73 | A | O |
| ATOM | 2721 | C | TYR | A | 374 | 42.685 | 51.726 | 92.247 | 1.00 | 85.38 | A | C |
| ATOM | 2722 | O | TYR | A | 374 | 43.223 | 52.755 | 92.662 | 1.00 | 85.28 | A | O |
| ATOM | 2723 | N | ASN | A | 375 | 41.430 | 51.401 | 92.545 | 1.00 | 90.38 | A | N |
| ATOM | 2724 | CA | ASN | A | 375 | 40.596 | 52.233 | 93.409 | 1.00 | 95.42 | A | C |
| ATOM | 2725 | CB | ASN | A | 375 | 39.820 | 53.264 | 92.580 | 1.00 | 95.00 | A | C |
| ATOM | 2726 | CG | ASN | A | 375 | 40.722 | 54.185 | 91.818 | 1.00 | 95.66 | A | C |
| ATOM | 2727 | OD1 | ASN | A | 375 | 41.694 | 54.697 | 92.367 | 1.00 | 96.47 | A | O |
| ATOM | 2728 | ND2 | ASN | A | 375 | 40.409 | 54.414 | 90.549 | 1.00 | 95.91 | A | N |
| ATOM | 2729 | C | ASN | A | 375 | 39.594 | 51.425 | 94.214 | 1.00 | 99.08 | A | C |
| ATOM | 2730 | O | ASN | A | 375 | 39.517 | 50.195 | 94.115 | 1.00 | 99.65 | A | O |
| ATOM | 2731 | N | LYS | A | 376 | 38.825 | 52.139 | 95.023 | 1.00 | 103.17 | A | N |
| ATOM | 2732 | CA | LYS | A | 376 | 37.808 | 51.486 | 95.802 | 1.00 | 107.54 | A | C |
| ATOM | 2733 | CB | LYS | A | 376 | 37.400 | 52.362 | 96.994 | 1.00 | 107.03 | A | C |
| ATOM | 2734 | CG | LYS | A | 376 | 38.559 | 52.465 | 97.974 | 1.00 | 107.83 | A | C |
| ATOM | 2735 | CD | LYS | A | 376 | 38.321 | 53.197 | 99.279 | 1.00 | 108.30 | A | C |
| ATOM | 2736 | CE | LYS | A | 376 | 39.597 | 53.091 | 100.124 | 1.00 | 108.72 | A | C |
| ATOM | 2737 | NZ | LYS | A | 376 | 39.566 | 53.799 | 101.449 | 1.00 | 108.51 | A | N |
| ATOM | 2738 | C | LYS | A | 376 | 36.687 | 51.204 | 94.830 | 1.00 | 111.02 | A | C |
| ATOM | 2739 | O | LYS | A | 376 | 35.860 | 50.321 | 95.036 | 1.00 | 112.01 | A | O |
| ATOM | 2740 | N | SER | A | 377 | 36.716 | 51.955 | 93.737 | 1.00 | 115.00 | A | N |
| ATOM | 2741 | CA | SER | A | 377 | 35.782 | 51.782 | 92.642 | 1.00 | 118.98 | A | C |
| ATOM | 2742 | CB | SER | A | 377 | 35.958 | 52.902 | 91.611 | 1.00 | 118.71 | A | C |
| ATOM | 2743 | OG | SER | A | 377 | 35.143 | 52.714 | 90.460 | 1.00 | 118.68 | A | O |
| ATOM | 2744 | C | SER | A | 377 | 36.058 | 50.437 | 91.981 | 1.00 | 121.83 | A | C |
| ATOM | 2745 | O | SER | A | 377 | 35.142 | 49.706 | 91.655 | 1.00 | 122.06 | A | O |
| ATOM | 2746 | N | ALA | A | 378 | 37.326 | 50.129 | 91.772 | 1.00 | 125.11 | A | N |
| ATOM | 2747 | CA | ALA | A | 378 | 37.710 | 48.878 | 91.162 | 1.00 | 128.59 | A | C |
| ATOM | 2748 | CB | ALA | A | 378 | 39.133 | 49.003 | 90.573 | 1.00 | 128.07 | A | C |
| ATOM | 2749 | C | ALA | A | 378 | 37.682 | 47.768 | 92.195 | 1.00 | 131.39 | A | C |
| ATOM | 2750 | O | ALA | A | 378 | 37.471 | 46.602 | 91.885 | 1.00 | 132.12 | A | O |
| ATOM | 2751 | N | TRP | A | 379 | 37.854 | 48.144 | 93.462 | 1.00 | 134.59 | A | N |
| ATOM | 2752 | CA | TRP | A | 379 | 37.902 | 47.152 | 94.607 | 1.00 | 137.82 | A | C |
| ATOM | 2753 | CB | TRP | A | 379 | 38.154 | 47.887 | 95.933 | 1.00 | 139.31 | A | C |
| ATOM | 2754 | CG | TRP | A | 379 | 38.047 | 47.075 | 97.167 | 1.00 | 141.20 | A | C |
| ATOM | 2755 | CD2 | TRP | A | 379 | 38.908 | 47.130 | 98.288 | 1.00 | 142.12 | A | C |
| ATOM | 2756 | CE2 | TRP | A | 379 | 38.388 | 46.247 | 99.273 | 1.00 | 142.56 | A | C |
| ATOM | 2757 | CE3 | TRP | A | 379 | 40.080 | 47.844 | 98.576 | 1.00 | 142.54 | A | C |
| ATOM | 2758 | CD1 | TRP | A | 379 | 37.054 | 46.170 | 97.496 | 1.00 | 141.92 | A | C |
| ATOM | 2759 | NE1 | TRP | A | 379 | 37.251 | 45.665 | 98.753 | 1.00 | 142.53 | A | N |
| ATOM | 2760 | CZ2 | TRP | A | 379 | 39.021 | 46.076 | 100.535 | 1.00 | 142.92 | A | C |
| ATOM | 2761 | CZ3 | TRP | A | 379 | 40.696 | 47.670 | 99.812 | 1.00 | 143.06 | A | C |
| ATOM | 2762 | CH2 | TRP | A | 379 | 40.176 | 46.809 | 100.770 | 1.00 | 143.24 | A | C |
| ATOM | 2763 | C | TRP | A | 379 | 36.458 | 46.546 | 94.607 | 1.00 | 139.46 | A | C |
| ATOM | 2764 | O | TRP | A | 379 | 36.242 | 45.276 | 94.615 | 1.00 | 139.58 | A | O |
| ATOM | 2765 | N | ARG | A | 380 | 35.475 | 47.454 | 94.527 | 1.00 | 141.59 | A | N |
| ATOM | 2766 | CA | ARG | A | 380 | 34.092 | 46.973 | 94.485 | 1.00 | 143.77 | A | C |
| ATOM | 2767 | CB | ARG | A | 380 | 33.151 | 48.116 | 94.898 | 1.00 | 143.92 | A | C |
| ATOM | 2768 | CG | ARG | A | 380 | 33.313 | 48.675 | 96.290 | 1.00 | 144.51 | A | C |
| ATOM | 2769 | CD | ARG | A | 380 | 32.471 | 49.919 | 96.563 | 1.00 | 145.73 | A | C |
| ATOM | 2770 | NE | ARG | A | 380 | 32.629 | 50.237 | 97.985 | 1.00 | 146.71 | A | N |
| ATOM | 2771 | CZ | ARG | A | 380 | 32.105 | 51.321 | 98.562 | 1.00 | 147.05 | A | C |
| ATOM | 2772 | NH1 | ARG | A | 380 | 32.314 | 51.592 | 99.855 | 1.00 | 147.12 | A | N |
| ATOM | 2773 | NH2 | ARG | A | 380 | 31.404 | 52.189 | 97.820 | 1.00 | 147.37 | A | N |
| ATOM | 2774 | C | ARG | A | 380 | 33.496 | 46.246 | 93.251 | 1.00 | 145.23 | A | C |
| ATOM | 2775 | O | ARG | A | 380 | 32.328 | 45.866 | 93.250 | 1.00 | 145.47 | A | O |
| ATOM | 2776 | N | HIS | A | 381 | 34.300 | 46.073 | 92.225 | 1.00 | 146.95 | A | N |
| ATOM | 2777 | CA | HIS | A | 381 | 33.858 | 45.403 | 91.051 | 1.00 | 148.84 | A | C |
| ATOM | 2778 | CB | HIS | A | 381 | 34.596 | 45.950 | 89.853 | 1.00 | 148.67 | A | C |
| ATOM | 2779 | CG | HIS | A | 381 | 33.678 | 46.445 | 88.774 | 1.00 | 148.86 | A | C |
| ATOM | 2780 | CD2 | HIS | A | 381 | 33.465 | 47.689 | 88.275 | 1.00 | 148.80 | A | C |
| ATOM | 2781 | ND1 | HIS | A | 381 | 32.876 | 45.599 | 88.044 | 1.00 | 148.86 | A | N |
| ATOM | 2782 | CE1 | HIS | A | 381 | 32.214 | 46.293 | 87.131 | 1.00 | 148.92 | A | C |
| ATOM | 2783 | NE2 | HIS | A | 381 | 32.554 | 47.561 | 87.250 | 1.00 | 148.93 | A | N |
| ATOM | 2784 | C | HIS | A | 381 | 34.035 | 43.889 | 91.120 | 1.00 | 150.08 | A | C |
| ATOM | 2785 | O | HIS | A | 381 | 33.591 | 43.163 | 90.272 | 1.00 | 150.13 | A | O |
| ATOM | 2786 | N | TYR | A | 382 | 34.654 | 43.386 | 92.163 | 1.00 | 151.55 | A | N |
| ATOM | 2787 | CA | TYR | A | 382 | 34.893 | 41.957 | 92.358 | 1.00 | 153.15 | A | C |
| ATOM | 2788 | CB | TYR | A | 382 | 36.349 | 41.768 | 92.768 | 1.00 | 152.39 | A | C |
| ATOM | 2789 | CG | TYR | A | 382 | 37.365 | 42.107 | 91.718 | 1.00 | 151.76 | A | C |
| ATOM | 2790 | CD1 | TYR | A | 382 | 37.425 | 41.402 | 90.510 | 1.00 | 151.28 | A | C |
| ATOM | 2791 | CE1 | TYR | A | 382 | 38.394 | 41.708 | 89.545 | 1.00 | 150.68 | A | C |
| ATOM | 2792 | CD2 | TYR | A | 382 | 38.286 | 43.118 | 91.938 | 1.00 | 151.22 | A | C |
| ATOM | 2793 | CE2 | TYR | A | 382 | 39.243 | 43.430 | 90.993 | 1.00 | 150.65 | A | C |
| ATOM | 2794 | CZ | TYR | A | 382 | 39.298 | 42.728 | 89.802 | 1.00 | 150.45 | A | C |
| ATOM | 2795 | OH | TYR | A | 382 | 40.271 | 43.066 | 88.889 | 1.00 | 149.75 | A | O |
| ATOM | 2796 | C | TYR | A | 382 | 34.008 | 41.478 | 93.526 | 1.00 | 154.71 | A | C |
| ATOM | 2797 | O | TYR | A | 382 | 34.299 | 40.456 | 94.128 | 1.00 | 154.85 | A | O |
| ATOM | 2798 | N | GLN | A | 383 | 33.002 | 42.267 | 93.908 | 1.00 | 156.62 | A | N |
| ATOM | 2799 | CA | GLN | A | 383 | 32.114 | 41.885 | 95.017 | 1.00 | 158.39 | A | C |
| ATOM | 2800 | CB | GLN | A | 383 | 31.767 | 43.112 | 95.882 | 1.00 | 157.81 | A | C |
| ATOM | 2801 | CG | GLN | A | 383 | 32.992 | 43.835 | 96.367 | 1.00 | 157.18 | A | C |
| ATOM | 2802 | CD | GLN | A | 383 | 34.005 | 42.882 | 96.988 | 1.00 | 156.82 | A | C |
| ATOM | 2803 | OE1 | GLN | A | 383 | 35.083 | 42.655 | 96.437 | 1.00 | 156.63 | A | O |
| ATOM | 2804 | NE2 | GLN | A | 383 | 33.657 | 42.319 | 98.139 | 1.00 | 156.72 | A | N |
| ATOM | 2805 | C | GLN | A | 383 | 30.830 | 41.210 | 94.528 | 1.00 | 159.98 | A | C |
| ATOM | 2806 | O | GLN | A | 383 | 29.885 | 41.022 | 95.293 | 1.00 | 160.32 | A | O |
| ATOM | 2807 | N | THR | A | 384 | 30.796 | 40.861 | 93.244 | 1.00 | 161.66 | A | N |
| ATOM | 2808 | CA | THR | A | 384 | 29.618 | 40.212 | 92.668 | 1.00 | 163.38 | A | C |
| ATOM | 2809 | CB | THR | A | 384 | 29.877 | 39.717 | 91.236 | 1.00 | 163.38 | A | C |
| ATOM | 2810 | OG1 | THR | A | 384 | 30.628 | 40.703 | 90.525 | 1.00 | 163.25 | A | O |
| ATOM | 2811 | CG2 | THR | A | 384 | 28.567 | 39.504 | 90.500 | 1.00 | 163.36 | A | C |
| ATOM | 2812 | C | THR | A | 384 | 29.208 | 39.025 | 93.533 | 1.00 | 164.54 | A | C |
| ATOM | 2813 | O | THR | A | 384 | 30.010 | 38.131 | 93.820 | 1.00 | 164.59 | A | O |
| ATOM | 2814 | N | ILE | A | 385 | 27.950 | 39.055 | 93.959 | 1.00 | 165.94 | A | N |
| ATOM | 2815 | CA | ILE | A | 385 | 27.364 | 38.038 | 94.819 | 1.00 | 167.29 | A | C |
| ATOM | 2816 | CB | ILE | A | 385 | 27.041 | 38.654 | 96.206 | 1.00 | 167.02 | A | C |
| ATOM | 2817 | CG2 | ILE | A | 385 | 26.300 | 37.650 | 97.088 | 1.00 | 167.10 | A | C |
| ATOM | 2818 | CG1 | ILE | A | 385 | 28.341 | 39.125 | 96.866 | 1.00 | 166.77 | A | C |
| ATOM | 2819 | CD1 | ILE | A | 385 | 28.132 | 39.935 | 98.123 | 1.00 | 166.17 | A | C |
| ATOM | 2820 | C | ILE | A | 385 | 26.083 | 37.563 | 94.143 | 1.00 | 168.29 | A | C |
| ATOM | 2821 | O | ILE | A | 385 | 25.045 | 37.400 | 94.782 | 1.00 | 168.52 | A | O |
| ATOM | 2822 | N | ALA | A | 386 | 26.172 | 37.339 | 92.836 | 1.00 | 169.36 | A | N |
| ATOM | 2823 | CA | ALA | A | 386 | 25.025 | 36.910 | 92.047 | 1.00 | 170.45 | A | C |
| ATOM | 2824 | CB | ALA | A | 386 | 25.425 | 36.783 | 90.589 | 1.00 | 170.18 | A | C |
| ATOM | 2825 | C | ALA | A | 386 | 24.334 | 35.626 | 92.508 | 1.00 | 171.35 | A | C |
| ATOM | 2826 | O | ALA | A | 386 | 24.974 | 34.666 | 92.943 | 1.00 | 171.44 | A | O |
| ATOM | 2827 | N | ALA | A | 387 | 23.009 | 35.638 | 92.386 | 1.00 | 172.25 | A | N |
| ATOM | 2828 | CA | ALA | A | 387 | 22.142 | 34.525 | 92.758 | 1.00 | 173.08 | A | C |
| ATOM | 2829 | CB | ALA | A | 387 | 22.441 | 34.074 | 94.183 | 1.00 | 172.89 | A | C |
| ATOM | 2830 | C | ALA | A | 387 | 20.703 | 35.026 | 92.652 | 1.00 | 173.76 | A | C |
| ATOM | 2831 | O | ALA | A | 387 | 20.093 | 35.385 | 93.659 | 1.00 | 174.00 | A | O |
| ATOM | 2832 | N | ALA | A | 388 | 20.189 | 35.051 | 91.421 | 1.00 | 174.48 | A | N |
| ATOM | 2833 | CA | ALA | A | 388 | 18.836 | 35.517 | 91.090 | 1.00 | 175.17 | A | C |
| ATOM | 2834 | CB | ALA | A | 388 | 17.902 | 35.427 | 92.308 | 1.00 | 175.21 | A | C |
| ATOM | 2835 | C | ALA | A | 388 | 18.888 | 36.956 | 90.569 | 1.00 | 175.56 | A | C |
| ATOM | 2836 | O | ALA | A | 388 | 19.190 | 37.887 | 91.317 | 1.00 | 175.88 | A | O |
| ATOM | 2837 | N | GLY | A | 389 | 18.586 | 37.130 | 89.284 | 1.00 | 175.80 | A | N |
| ATOM | 2838 | CA | GLY | A | 389 | 18.625 | 38.451 | 88.677 | 1.00 | 175.97 | A | C |
| ATOM | 2839 | C | GLY | A | 389 | 17.410 | 39.345 | 88.857 | 1.00 | 176.17 | A | C |
| ATOM | 2840 | O | GLY | A | 389 | 16.484 | 39.032 | 89.609 | 1.00 | 176.43 | A | O |
| ATOM | 2841 | N | ASP | A | 390 | 17.426 | 40.471 | 88.146 | 1.00 | 176.11 | A | N |
| ATOM | 2842 | CA | ASP | A | 390 | 16.356 | 41.464 | 88.198 | 1.00 | 175.90 | A | C |
| ATOM | 2843 | CB | ASP | A | 390 | 16.960 | 42.880 | 88.141 | 1.00 | 175.61 | A | C |
| ATOM | 2844 | CG | ASP | A | 390 | 18.108 | 43.000 | 87.139 | 1.00 | 175.34 | A | C |
| ATOM | 2845 | OD1 | ASP | A | 390 | 18.625 | 41.958 | 86.686 | 1.00 | 175.36 | A | O |
| ATOM | 2846 | OD2 | ASP | A | 390 | 18.510 | 44.139 | 86.818 | 1.00 | 174.83 | A | O |
| ATOM | 2847 | C | ASP | A | 390 | 15.297 | 41.287 | 87.108 | 1.00 | 175.87 | A | C |
| ATOM | 2848 | O | ASP | A | 390 | 14.276 | 41.980 | 87.104 | 1.00 | 175.50 | A | O |
| ATOM | 2849 | N | TRP | A | 391 | 15.546 | 40.357 | 86.190 | 1.00 | 175.98 | A | N |
| ATOM | 2850 | CA | TRP | A | 391 | 14.608 | 40.065 | 85.111 | 1.00 | 176.09 | A | C |
| ATOM | 2851 | CB | TRP | A | 391 | 15.258 | 40.313 | 83.741 | 1.00 | 175.90 | A | C |
| ATOM | 2852 | CG | TRP | A | 391 | 15.948 | 41.649 | 83.609 | 1.00 | 175.70 | A | C |
| ATOM | 2853 | CD2 | TRP | A | 391 | 15.460 | 42.806 | 82.918 | 1.00 | 175.55 | A | C |
| ATOM | 2854 | CE2 | TRP | A | 391 | 16.424 | 43.828 | 83.078 | 1.00 | 175.41 | A | C |
| ATOM | 2855 | CE3 | TRP | A | 391 | 14.301 | 43.081 | 82.181 | 1.00 | 175.45 | A | C |
| ATOM | 2856 | CD1 | TRP | A | 391 | 17.154 | 42.004 | 84.142 | 1.00 | 175.68 | A | C |
| ATOM | 2857 | NE1 | TRP | A | 391 | 17.448 | 43.311 | 83.828 | 1.00 | 175.53 | A | N |
| ATOM | 2858 | CZ2 | TRP | A | 391 | 16.266 | 45.103 | 82.528 | 1.00 | 175.08 | A | C |
| ATOM | 2859 | CZ3 | TRP | A | 391 | 14.143 | 44.352 | 81.633 | 1.00 | 175.20 | A | C |
| ATOM | 2860 | CH2 | TRP | A | 391 | 15.122 | 45.345 | 81.812 | 1.00 | 174.90 | A | C |
| ATOM | 2861 | C | TRP | A | 391 | 14.203 | 38.593 | 85.239 | 1.00 | 176.30 | A | C |
| ATOM | 2862 | O | TRP | A | 391 | 14.952 | 37.788 | 85.798 | 1.00 | 176.27 | A | O |
| ATOM | 2863 | N | CYS | A | 392 | 13.020 | 38.241 | 84.738 | 1.00 | 176.58 | A | N |
| ATOM | 2864 | CA | CYS | A | 392 | 12.550 | 36.856 | 84.816 | 1.00 | 176.70 | A | C |
| ATOM | 2865 | CB | CYS | A | 392 | 11.406 | 36.726 | 85.831 | 1.00 | 176.51 | A | C |
| ATOM | 2866 | SG | CYS | A | 392 | 9.778 | 37.297 | 85.263 | 1.00 | 175.89 | A | S |
| ATOM | 2867 | C | CYS | A | 392 | 12.092 | 36.321 | 83.459 | 1.00 | 176.83 | A | C |
| ATOM | 2868 | O | CYS | A | 392 | 12.913 | 36.336 | 82.519 | 1.00 | 177.02 | A | O |
| ATOM | 2869 | OXT | CYS | A | 392 | 10.926 | 35.881 | 83.352 | 1.00 | 176.87 | A | O |
| TER | 2870 | | CYS | A | 392 | | | | | | A | |
| ATOM | 2871 | O | HOH | W | 1 | 46.356 | 50.937 | 69.793 | 1.00 | 21.08 | W | O |
| ATOM | 2872 | O | HOH | W | 2 | 58.863 | 57.534 | 76.329 | 1.00 | 38.95 | W | O |
| ATOM | 2873 | O | HOH | W | 3 | 35.793 | 36.005 | 80.336 | 1.00 | 21.57 | W | O |
| ATOM | 2874 | O | HOH | W | 4 | 32.568 | 56.545 | 63.324 | 1.00 | 18.77 | W | O |
| ATOM | 2875 | O | HOH | W | 5 | 28.791 | 53.631 | 65.723 | 1.00 | 20.74 | W | O |
| ATOM | 2876 | O | HOH | W | 6 | 39.877 | 62.413 | 66.812 | 1.00 | 30.22 | W | O |
| ATOM | 2877 | O | HOH | W | 7 | 30.537 | 44.114 | 69.490 | 1.00 | 34.10 | W | O |
| ATOM | 2878 | O | HOH | W | 8 | 33.726 | 59.524 | 68.508 | 1.00 | 29.79 | W | O |
| ATOM | 2879 | O | HOH | W | 9 | 54.528 | 43.101 | 79.867 | 1.00 | 36.50 | W | O |
| ATOM | 2880 | O | HOH | W | 10 | 34.801 | 22.345 | 91.701 | 1.00 | 38.56 | W | O |
| ATOM | 2881 | O | HOH | W | 11 | 43.129 | 53.234 | 69.331 | 1.00 | 21.83 | W | O |
| ATOM | 2882 | O | HOH | W | 12 | 42.325 | 55.917 | 68.566 | 1.00 | 19.18 | W | O |
| ATOM | 2883 | O | HOH | W | 13 | 38.159 | 22.001 | 93.193 | 1.00 | 29.07 | W | O |
| ATOM | 2884 | O | HOH | W | 14 | 30.274 | 35.964 | 58.692 | 1.00 | 26.54 | W | O |
| ATOM | 2885 | O | HOH | W | 15 | 43.913 | 57.370 | 67.232 | 1.00 | 21.35 | W | O |
| ATOM | 2886 | O | HOH | W | 16 | 36.158 | 39.739 | 60.659 | 1.00 | 19.01 | W | O |
| ATOM | 2887 | O | HOH | W | 17 | 50.846 | 43.173 | 67.041 | 1.00 | 39.61 | W | O |
| ATOM | 2888 | O | HOH | W | 18 | 30.335 | 55.703 | 64.792 | 1.00 | 22.71 | W | O |
| ATOM | 2889 | O | HOH | W | 19 | 48.338 | 27.813 | 86.695 | 1.00 | 34.06 | W | O |
| ATOM | 2890 | O | HOH | W | 20 | 45.129 | 25.623 | 88.697 | 1.00 | 35.37 | W | O |
| ATOM | 2891 | O | HOH | W | 21 | 33.028 | 45.335 | 71.142 | 1.00 | 29.88 | W | O |
| ATOM | 2892 | O | HOH | W | 22 | 34.363 | 40.684 | 58.767 | 1.00 | 25.96 | W | O |
| ATOM | 2893 | O | HOH | W | 23 | 36.801 | 36.265 | 46.234 | 1.00 | 37.61 | W | O |
| ATOM | 2894 | O | HOH | W | 24 | 35.819 | 44.905 | 80.674 | 1.00 | 37.56 | W | O |
| ATOM | 2895 | O | HOH | W | 25 | 28.132 | 51.639 | 67.813 | 1.00 | 27.55 | W | O |
| ATOM | 2896 | O | HOH | W | 26 | 45.493 | 23.034 | 79.484 | 1.00 | 66.23 | W | O |
| ATOM | 2897 | O | HOH | W | 27 | 33.307 | 36.929 | 63.181 | 1.00 | 49.28 | W | O |
| ATOM | 2898 | O | HOH | W | 28 | 44.332 | 62.397 | 55.973 | 1.00 | 31.16 | W | O |
| ATOM | 2899 | O | HOH | W | 29 | 28.040 | 50.907 | 59.889 | 1.00 | 25.61 | W | O |
| ATOM | 2900 | O | HOH | W | 30 | 36.988 | 45.842 | 76.165 | 1.00 | 36.63 | W | O |
| ATOM | 2901 | O | HOH | W | 31 | 57.671 | 35.319 | 79.039 | 1.00 | 32.23 | W | O |
| ATOM | 2902 | O | HOH | W | 32 | 28.755 | 41.979 | 55.934 | 1.00 | 32.70 | W | O |
| ATOM | 2903 | O | HOH | W | 33 | 35.990 | 38.645 | 63.384 | 1.00 | 27.77 | W | O |
| ATOM | 2904 | O | HOH | W | 34 | 32.653 | 63.946 | 59.181 | 1.00 | 35.34 | W | O |
| ATOM | 2905 | O | HOH | W | 35 | 40.938 | 33.247 | 101.661 | 1.00 | 55.40 | W | O |
| ATOM | 2906 | O | HOH | W | 36 | 52.023 | 58.399 | 87.251 | 1.00 | 35.31 | W | O |
| ATOM | 2907 | O | HOH | W | 37 | 38.872 | 65.301 | 57.787 | 1.00 | 31.90 | W | O |
| ATOM | 2908 | O | HOH | W | 38 | 40.898 | 64.888 | 74.639 | 1.00 | 58.18 | W | O |
| ATOM | 2909 | O | HOH | W | 39 | 26.535 | 48.257 | 55.274 | 1.00 | 29.53 | W | O |
| ATOM | 2910 | O | HOH | W | 40 | 36.629 | 57.028 | 67.242 | 1.00 | 33.37 | W | O |
| ATOM | 2911 | O | HOH | W | 41 | 49.159 | 23.607 | 92.976 | 1.00 | 54.60 | W | O |
| ATOM | 2912 | O | HOH | W | 42 | 60.814 | 53.467 | 97.902 | 1.00 | 54.52 | W | O |
| ATOM | 2913 | O | HOH | W | 43 | 48.397 | 33.474 | 63.243 | 1.00 | 36.55 | W | O |
| ATOM | 2914 | O | HOH | W | 44 | 33.336 | 37.247 | 52.717 | 1.00 | 35.50 | W | O |
| ATOM | 2915 | O | HOH | W | 45 | 79.783 | 55.630 | 85.197 | 1.00 | 48.10 | W | O |
| ATOM | 2916 | O | HOH | W | 46 | 34.706 | 57.784 | 70.864 | 1.00 | 35.72 | W | O |
| ATOM | 2917 | O | HOH | W | 47 | 34.773 | 57.527 | 60.425 | 1.00 | 31.00 | W | O |
| ATOM | 2918 | O | HOH | W | 48 | 52.003 | 37.328 | 75.874 | 1.00 | 35.31 | W | O |
| ATOM | 2919 | O | HOH | W | 49 | 43.009 | 31.404 | 58.514 | 1.00 | 48.66 | W | O |
| ATOM | 2920 | O | HOH | W | 50 | 38.482 | 43.326 | 75.229 | 1.00 | 39.26 | W | O |
| ATOM | 2921 | O | HOH | W | 51 | 36.818 | 19.114 | 86.589 | 1.00 | 27.25 | W | O |
| ATOM | 2922 | O | HOH | W | 52 | 46.301 | 49.554 | 86.999 | 1.00 | 50.93 | W | O |
| ATOM | 2923 | O | HOH | W | 53 | 46.134 | 60.091 | 66.086 | 1.00 | 32.98 | W | O |
| ATOM | 2924 | O | HOH | W | 54 | 35.225 | 54.231 | 73.628 | 1.00 | 50.49 | W | O |
| ATOM | 2925 | O | HOH | W | 55 | 54.337 | 64.076 | 81.603 | 1.00 | 45.61 | W | O |
| ATOM | 2926 | O | HOH | W | 56 | 47.738 | 64.614 | 71.454 | 1.00 | 50.44 | W | O |
| ATOM | 2927 | O | HOH | W | 57 | 48.738 | 61.923 | 59.663 | 1.00 | 34.08 | W | O |
| ATOM | 2928 | O | HOH | W | 58 | 45.960 | 61.332 | 63.787 | 1.00 | 32.12 | W | O |
| ATOM | 2929 | O | HOH | W | 59 | 51.493 | 39.927 | 73.964 | 1.00 | 39.84 | W | O |
| ATOM | 2930 | O | HOH | W | 60 | 46.713 | 39.487 | 65.763 | 1.00 | 35.71 | W | O |
| ATOM | 2931 | O | HOH | W | 61 | 65.836 | 44.221 | 83.635 | 1.00 | 38.25 | W | O |
| ATOM | 2932 | O | HOH | W | 62 | 28.808 | 52.014 | 70.822 | 1.00 | 37.24 | W | O |
| ATOM | 2933 | O | HOH | W | 63 | 39.839 | 61.670 | 73.915 | 1.00 | 45.35 | W | O |
| ATOM | 2934 | O | HOH | W | 64 | 60.285 | 34.633 | 76.912 | 1.00 | 52.43 | W | O |
| ATOM | 2935 | O | HOH | W | 65 | 51.685 | 64.174 | 60.883 | 1.00 | 46.93 | W | O |
| ATOM | 2936 | O | HOH | W | 66 | 46.222 | 64.239 | 62.363 | 1.00 | 42.04 | W | O |
| ATOM | 2937 | O | HOH | W | 67 | 32.132 | 65.041 | 64.854 | 1.00 | 38.75 | W | O |
| ATOM | 2938 | O | HOH | W | 68 | 48.953 | 41.877 | 74.079 | 1.00 | 35.38 | W | O |
| ATOM | 2939 | O | HOH | W | 69 | 57.465 | 31.412 | 92.002 | 1.00 | 44.74 | W | O |
| ATOM | 2940 | O | HOH | W | 70 | 32.604 | 47.933 | 73.300 | 1.00 | 48.95 | W | O |
| ATOM | 2941 | O | HOH | W | 71 | 51.752 | 24.351 | 77.939 | 1.00 | 35.85 | W | O |
| ATOM | 2942 | O | HOH | W | 72 | 41.750 | 34.875 | 64.258 | 1.00 | 41.32 | W | O |
| ATOM | 2943 | O | HOH | W | 73 | 32.519 | 41.974 | 83.213 | 1.00 | 38.98 | W | O |
| ATOM | 2944 | O | HOH | W | 74 | 34.040 | 21.791 | 84.421 | 1.00 | 60.50 | W | O |
| ATOM | 2945 | O | HOH | W | 75 | 69.155 | 53.444 | 92.803 | 1.00 | 49.16 | W | O |
| ATOM | 2946 | O | HOH | W | 76 | 32.791 | 47.478 | 54.192 | 1.00 | 27.63 | W | O |
| ATOM | 2947 | O | HOH | W | 77 | 73.267 | 45.959 | 81.683 | 1.00 | 41.49 | W | O |
| ATOM | 2948 | O | HOH | W | 78 | 27.900 | 20.294 | 47.217 | 1.00 | 62.93 | W | O |
| ATOM | 2949 | O | HOH | W | 79 | 68.411 | 55.293 | 78.447 | 1.00 | 37.08 | W | O |
| ATOM | 2950 | O | HOH | W | 80 | 28.220 | 30.640 | 82.093 | 1.00 | 63.49 | W | O |
| ATOM | 2951 | O | HOH | W | 81 | 46.100 | 38.304 | 69.589 | 1.00 | 56.99 | W | O |
| ATOM | 2952 | O | HOH | W | 82 | 31.058 | 27.357 | 88.569 | 1.00 | 47.16 | W | O |
| ATOM | 2953 | O | HOH | W | 83 | 77.763 | 54.080 | 77.151 | 1.00 | 48.60 | W | O |
| ATOM | 2954 | O | HOH | W | 84 | 42.492 | 40.513 | 46.027 | 1.00 | 42.89 | W | O |
| ATOM | 2955 | O | HOH | W | 85 | 60.650 | 32.468 | 85.303 | 1.00 | 47.15 | W | O |
| ATOM | 2956 | O | HOH | W | 86 | 60.000 | 55.898 | 56.908 | 1.00 | 58.56 | W | O |
| ATOM | 2957 | O | HOH | W | 87 | 36.114 | 64.221 | 72.976 | 1.00 | 52.14 | W | O |
| ATOM | 2958 | O | HOH | W | 88 | 32.386 | 42.009 | 76.221 | 1.00 | 41.39 | W | O |
| ATOM | 2959 | O | HOH | W | 89 | 68.840 | 41.169 | 84.966 | 1.00 | 48.93 | W | O |
| ATOM | 2960 | O | HOH | W | 90 | 48.314 | 31.777 | 65.151 | 1.00 | 45.02 | W | O |
| ATOM | 2961 | O | HOH | W | 91 | 31.635 | 43.079 | 44.213 | 1.00 | 60.63 | W | O |
| ATOM | 2962 | O | HOH | W | 92 | 34.393 | 24.137 | 80.991 | 1.00 | 56.58 | W | O |
| ATOM | 2963 | O | HOH | W | 93 | 31.366 | 34.503 | 60.779 | 1.00 | 33.75 | W | O |
| ATOM | 2964 | O | HOH | W | 94 | 69.405 | 43.479 | 77.954 | 1.00 | 55.90 | W | O |
| ATOM | 2965 | O | HOH | W | 95 | 47.216 | 34.704 | 66.672 | 1.00 | 53.52 | W | O |
| ATOM | 2966 | O | HOH | W | 96 | 60.738 | 33.566 | 87.757 | 1.00 | 46.43 | W | O |
| ATOM | 2967 | O | HOH | W | 97 | 54.113 | 49.142 | 70.751 | 1.00 | 46.93 | W | O |
| ATOM | 2968 | O | HOH | W | 98 | 35.198 | 43.198 | 77.636 | 1.00 | 42.16 | W | O |
| ATOM | 2969 | O | HOH | W | 99 | 40.429 | 65.105 | 55.506 | 1.00 | 46.73 | W | O |
| ATOM | 2970 | O | HOH | W | 100 | 60.436 | 40.749 | 98.158 | 1.00 | 40.10 | W | O |
| ATOM | 2971 | O | HOH | W | 101 | 52.222 | 47.589 | 72.946 | 1.00 | 56.04 | W | O |
| ATOM | 2972 | O | HOH | W | 102 | 31.683 | 38.072 | 78.315 | 1.00 | 54.26 | W | O |
| ATOM | 2973 | O | HOH | W | 103 | 60.208 | 38.473 | 101.048 | 1.00 | 50.25 | W | O |
| ATOM | 2974 | O | HOH | W | 104 | 32.233 | 39.467 | 76.290 | 1.00 | 36.67 | W | O |
| ATOM | 2975 | O | HOH | W | 105 | 39.067 | 66.914 | 61.956 | 1.00 | 42.51 | W | O |
| ATOM | 2976 | O | HOH | W | 106 | 26.862 | 31.349 | 53.718 | 1.00 | 53.79 | W | O |
| ATOM | 2977 | O | HOH | W | 107 | 50.342 | 65.461 | 65.489 | 1.00 | 42.84 | W | O |
| ATOM | 2978 | O | HOH | W | 108 | 47.396 | 63.435 | 56.168 | 1.00 | 53.57 | W | O |
| ATOM | 2979 | O | HOH | W | 109 | 67.116 | 40.861 | 86.831 | 1.00 | 45.87 | W | O |
| ATOM | 2980 | O | HOH | W | 110 | 63.445 | 35.909 | 90.394 | 1.00 | 47.03 | W | O |
| ATOM | 2981 | O | HOH | W | 111 | 57.881 | 58.434 | 72.042 | 1.00 | 26.42 | W | O |
| ATOM | 2982 | O | HOH | W | 112 | 58.459 | 40.368 | 101.290 | 1.00 | 43.22 | W | O |
| ATOM | 2983 | O | HOH | W | 113 | 32.285 | 66.756 | 55.663 | 1.00 | 49.03 | W | O |
| ATOM | 2984 | O | HOH | W | 114 | 39.511 | 64.439 | 60.191 | 1.00 | 36.31 | W | O |
| ATOM | 2985 | O | HOH | W | 115 | 39.126 | 31.465 | 97.026 | 1.00 | 44.70 | W | O |
| ATOM | 2986 | O | HOH | W | 116 | 60.462 | 58.027 | 73.073 | 1.00 | 48.95 | W | O |
| ATOM | 2987 | O | HOH | W | 117 | 26.629 | 19.051 | 48.992 | 1.00 | 60.38 | W | O |
| ATOM | 2988 | O | HOH | W | 118 | 58.836 | 63.938 | 66.754 | 1.00 | 50.78 | W | O |
| ATOM | 2989 | O | HOH | W | 119 | 27.085 | 25.669 | 85.852 | 1.00 | 52.47 | W | O |
| ATOM | 2990 | O | HOH | W | 120 | 27.999 | 52.940 | 57.304 | 1.00 | 48.81 | W | O |
| ATOM | 2991 | O | HOH | W | 121 | 40.659 | 58.810 | 49.907 | 1.00 | 42.86 | W | O |
| ATOM | 2992 | O | HOH | W | 122 | 58.335 | 29.925 | 89.236 | 1.00 | 57.60 | W | O |
| ATOM | 2993 | O | HOH | W | 123 | 68.795 | 61.634 | 90.448 | 1.00 | 53.87 | W | O |
| ATOM | 2994 | O | HOH | W | 124 | 50.968 | 27.554 | 92.690 | 1.00 | 51.94 | W | O |
| ATOM | 2995 | O | HOH | W | 125 | 56.191 | 39.418 | 96.860 | 1.00 | 54.80 | W | O |
| ATOM | 2996 | O | HOH | W | 126 | 63.391 | 37.971 | 99.661 | 1.00 | 60.70 | W | O |
| ATOM | 2997 | O | HOH | W | 127 | 69.314 | 40.119 | 76.758 | 1.00 | 57.13 | W | O |
| ATOM | 2998 | O | HOH | W | 128 | 68.001 | 43.733 | 89.340 | 1.00 | 42.10 | W | O |
| ATOM | 2999 | O | HOH | W | 129 | 43.795 | 66.229 | 51.977 | 1.00 | 55.97 | W | O |
| ATOM | 3000 | O | HOH | W | 130 | 40.689 | 30.787 | 63.008 | 1.00 | 62.91 | W | O |
| ATOM | 3001 | O | HOH | W | 131 | 48.439 | 28.388 | 77.254 | 1.00 | 48.41 | W | O |
| ATOM | 3002 | O | HOH | W | 132 | 32.633 | 69.455 | 58.449 | 1.00 | 64.65 | W | O |
| ATOM | 3003 | O | HOH | W | 133 | 36.664 | 17.828 | 84.178 | 1.00 | 46.17 | W | O |
| ATOM | 3004 | O | HOH | W | 134 | 39.457 | 34.913 | 67.732 | 1.00 | 43.40 | W | O |
| ATOM | 3005 | O | HOH | W | 135 | 64.595 | 36.628 | 85.561 | 1.00 | 56.88 | W | O |
| ATOM | 3006 | O | HOH | W | 136 | 62.574 | 59.656 | 88.977 | 1.00 | 59.11 | W | O |
| ATOM | 3007 | O | HOH | W | 137 | 36.614 | 63.744 | 56.590 | 1.00 | 37.03 | W | O |
| ATOM | 3008 | O | HOH | W | 138 | 45.556 | 37.070 | 66.110 | 1.00 | 41.75 | W | O |
| ATOM | 3009 | O | HOH | W | 139 | 17.039 | 48.920 | 82.632 | 1.00 | 51.72 | W | O |
| ATOM | 3010 | O | HOH | W | 140 | 29.014 | 50.771 | 55.951 | 1.00 | 34.92 | W | O |
| ATOM | 3011 | O | HOH | W | 141 | 34.238 | 37.493 | 60.437 | 1.00 | 33.76 | W | O |
| ATOM | 3012 | O | HOH | W | 142 | 36.443 | 56.023 | 69.664 | 1.00 | 42.15 | W | O |
| ATOM | 3013 | O | HOH | W | 143 | 47.917 | 63.565 | 78.461 | 1.00 | 44.32 | W | O |
| ATOM | 3014 | O | HOH | W | 144 | 26.223 | 50.478 | 54.422 | 1.00 | 47.63 | W | O |
| ATOM | 3015 | O | HOH | W | 145 | 38.126 | 62.872 | 72.466 | 1.00 | 63.39 | W | O |
| ATOM | 3016 | O | HOH | W | 146 | 42.576 | 56.749 | 47.238 | 1.00 | 54.27 | W | O |
| ATOM | 3017 | O | HOH | W | 147 | 65.706 | 36.769 | 101.888 | 1.00 | 49.95 | W | O |
| ATOM | 3018 | O | HOH | W | 148 | 38.103 | 59.251 | 49.469 | 1.00 | 38.09 | W | O |
| ATOM | 3019 | O | HOH | W | 149 | 54.136 | 39.710 | 74.044 | 1.00 | 44.16 | W | O |
| ATOM | 3020 | O | HOH | W | 150 | 42.733 | 64.250 | 54.095 | 1.00 | 49.60 | W | O |
| ATOM | 3021 | O | HOH | W | 151 | 34.832 | 34.452 | 63.934 | 1.00 | 53.36 | W | O |
| ATOM | 3022 | O | HOH | W | 152 | 35.750 | 32.732 | 62.022 | 1.00 | 40.89 | W | O |
| ATOM | 3023 | O | HOH | W | 153 | 34.067 | 30.007 | 60.783 | 1.00 | 58.11 | W | O |
| TER | 3024 | | HOH | W | 153 | | | | | | W | |
| ATOM | 3025 | C1 | HET | B | 1 | 36.997 | 35.499 | 90.153 | 1.00 | 55.65 | B | C |
| ATOM | 3026 | C2 | HET | B | 1 | 36.816 | 36.679 | 90.932 | 1.00 | 55.45 | B | C |
| ATOM | 3027 | C3 | HET | B | 1 | 35.971 | 34.515 | 90.233 | 1.00 | 57.79 | B | C |
| ATOM | 3028 | O1 | HET | B | 1 | 38.171 | 35.381 | 89.364 | 1.00 | 52.95 | B | O |
| ATOM | 3029 | C4 | HET | B | 1 | 35.679 | 36.881 | 91.733 | 1.00 | 59.33 | B | C |
| ATOM | 3030 | CL1 | HET | B | 1 | 37.990 | 37.937 | 90.918 | 1.00 | 52.19 | B | CL |
| ATOM | 3031 | C5 | HET | B | 1 | 34.836 | 34.713 | 91.038 | 1.00 | 60.59 | B | C |
| ATOM | 3032 | C6 | HET | B | 1 | 38.347 | 34.229 | 88.554 | 1.00 | 47.91 | B | C |
| ATOM | 3033 | C7 | HET | B | 1 | 34.675 | 35.883 | 91.792 | 1.00 | 62.61 | B | C |
| ATOM | 3034 | C8 | HET | B | 1 | 39.641 | 34.318 | 87.779 | 1.00 | 45.34 | B | C |
| ATOM | 3035 | C9 | HET | B | 1 | 33.443 | 36.054 | 92.655 | 1.00 | 67.68 | B | C |
| ATOM | 3036 | C10 | HET | B | 1 | 40.122 | 33.188 | 87.101 | 1.00 | 42.18 | B | C |
| ATOM | 3037 | C11 | HET | B | 1 | 40.401 | 35.532 | 87.710 | 1.00 | 43.11 | B | C |
| ATOM | 3038 | C12 | HET | B | 1 | 33.707 | 35.655 | 94.139 | 1.00 | 72.64 | B | C |
| ATOM | 3039 | C13 | HET | B | 1 | 41.332 | 33.259 | 86.371 | 1.00 | 42.72 | B | C |
| ATOM | 3040 | C14 | HET | B | 1 | 41.609 | 35.606 | 86.983 | 1.00 | 43.22 | B | C |
| ATOM | 3041 | N1 | HET | B | 1 | 34.565 | 36.503 | 94.790 | 1.00 | 76.05 | B | N |
| ATOM | 3042 | O2 | HET | B | 1 | 33.171 | 34.660 | 94.652 | 1.00 | 73.98 | B | O |
| ATOM | 3043 | C15 | HET | B | 1 | 42.097 | 34.474 | 86.300 | 1.00 | 42.83 | B | C |
| ATOM | 3044 | C16 | HET | B | 1 | 34.963 | 36.329 | 96.204 | 1.00 | 79.37 | B | C |
| ATOM | 3045 | C17 | HET | B | 1 | 43.391 | 34.576 | 85.513 | 1.00 | 44.18 | B | C |
| ATOM | 3046 | C18 | HET | B | 1 | 36.256 | 35.527 | 96.261 | 1.00 | 80.83 | B | C |
| ATOM | 3047 | C19 | HET | B | 1 | 44.126 | 33.234 | 85.445 | 1.00 | 43.96 | B | C |
| ATOM | 3048 | C20 | HET | B | 1 | 43.073 | 35.119 | 84.109 | 1.00 | 45.48 | B | C |
| ATOM | 3049 | N2 | HET | B | 1 | 36.846 | 35.540 | 97.590 | 1.00 | 81.36 | B | N |
| TER | 3050 | | HET | B | 1 | | | | | | B | |
| END | | | | | | | | | | | | |

TABLE 8
Atomic coordinates for PDHK-2* with DCA and ADP soaked in

REMARK coordinates from restrained individual B-factor refinement
REMARK refinement resolution: 30 − 2.5 A

| | | |
|---|---|---|
| REMARK starting | r = 0.2626 | free_r = 0.2616 |
| REMARK final | r = 0.2354 | free_r = 0.2475 |

| | | |
|---|---|---|
| REMARK B rmsd for bonded mainchain atoms = | 2.557 | target = 1.5 |
| REMARK B rmsd for bonded sidechain atoms = | 3.430 | target = 2.0 |
| REMARK B rmsd for angle mainchain atoms = | 4.180 | target = 2.0 |
| REMARK B rmsd for angle sidechain atoms = | 4.966 | target = 2.5 |

REMARK wa = 2.5139
REMARK rweight = 5E−02
REMARK target = mlf steps = 30
REMARK sg = P6(4) a = 110.699 b = 110.699 c = 84.609 alpha = 90 beta = 90 gamma = 120
REMARK parameter file 1: MSI_CNX_TOPPAR:protein_rep.param
REMARK parameter file 2: ../toppar/het.par
REMARK parameter file 3: MSI_CNX_TOPPAR:water_rep.param
REMARK parameter file 4: MSI_CNX_TOPPAR:ion.param
REMARK parameter file 5: ../toppar/adp.par
REMARK molecular structure file: ../generate/generate.psf
REMARK input coordinates: minimize.pdb
REMARK reflection file = ../pdhk107.cv
REMARK ncs = none
REMARK B-correction resolution: 6.0 − 2.5
REMARK initial B-factor correction applied to fobs:

| | | | | | | |
|---|---|---|---|---|---|---|
| REMARK | B11 = | −2.490 | B22 = | −2.490 | B33 = | 4.980 |
| REMARK | B12 = | −6.188 | B13 = | 0.000 | B23 = | 0.000 |

REMARK B-factor correction applied to coordinate array B: −0.068
REMARK bulk solvent: (Mask) density level = 0.357975 e/A\hat{ }3, B-factor = 68.5677 A\hat{ }2
REMARK reflections with |Fobs|/sigma_F < 0.0 rejected
REMARK reflections with |Fobs| > 10000 * rms(Fobs) rejected

| | | |
|---|---|---|
| REMARK theoretical total number of refl. in resol. range: | 20486 | (100.0%) |
| REMARK number of unobserved reflections (no entry or \|F\| = 0): | 156 | (0.8%) |
| REMARK number of reflections rejected: | 0 | (0.0%) |
| REMARK total number of reflections used: | 20330 | (99.2%) |
| REMARK number of reflections in working set: | 19632 | (95.8%) |
| REMARK number of reflections in test set: | 698 | (3.4%) |
| REMARK FILENAME = “bindividual.pdb” | | |
| REMARK DATE: May 02, 2001 16:38:12 created by user: knoechel | | |
| REMARK Written by CNX VERSION: 2000 | | |

| | | | | | | | | | | | |
|---|---|---|---|---|---|---|---|---|---|---|---|
| ATOM | 1 | C | GLY | A | 14 | 62.539 | 43.177 | 101.294 | 1.00 | 69.52 | C |
| ATOM | 2 | O | GLY | A | 14 | 61.906 | 43.352 | 102.335 | 1.00 | 68.76 | O |
| ATOM | 3 | N | GLY | A | 14 | 64.545 | 42.189 | 100.211 | 1.00 | 71.25 | N |
| ATOM | 4 | CA | GLY | A | 14 | 63.391 | 41.937 | 101.105 | 1.00 | 67.67 | C |
| ATOM | 5 | N | SER | A | 15 | 62.504 | 44.043 | 100.281 | 1.00 | 71.41 | N |
| ATOM | 6 | CA | SER | A | 15 | 61.716 | 45.280 | 100.356 | 1.00 | 75.16 | C |
| ATOM | 7 | CB | SER | A | 15 | 62.652 | 46.481 | 100.590 | 1.00 | 77.64 | C |
| ATOM | 8 | OG | SER | A | 15 | 63.552 | 46.676 | 99.508 | 1.00 | 80.99 | O |
| ATOM | 9 | C | SER | A | 15 | 60.785 | 45.584 | 99.163 | 1.00 | 73.53 | C |
| ATOM | 10 | O | SER | A | 15 | 60.320 | 46.712 | 99.016 | 1.00 | 72.09 | O |
| ATOM | 11 | N | ALA | A | 16 | 60.510 | 44.591 | 98.321 | 1.00 | 73.17 | N |
| ATOM | 12 | CA | ALA | A | 16 | 59.623 | 44.799 | 97.175 | 1.00 | 72.79 | C |
| ATOM | 13 | CB | ALA | A | 16 | 59.537 | 43.536 | 96.329 | 1.00 | 72.45 | C |
| ATOM | 14 | C | ALA | A | 16 | 58.221 | 45.216 | 97.634 | 1.00 | 72.17 | C |
| ATOM | 15 | O | ALA | A | 16 | 57.601 | 46.088 | 97.027 | 1.00 | 70.79 | O |
| ATOM | 16 | N | PRO | A | 17 | 57.698 | 44.592 | 98.706 | 1.00 | 71.68 | N |
| ATOM | 17 | CD | PRO | A | 17 | 58.166 | 43.366 | 99.378 | 1.00 | 70.97 | C |
| ATOM | 18 | CA | PRO | A | 17 | 56.363 | 44.960 | 99.189 | 1.00 | 72.74 | C |
| ATOM | 19 | CB | PRO | A | 17 | 56.160 | 44.021 | 100.374 | 1.00 | 70.71 | C |
| ATOM | 20 | CG | PRO | A | 17 | 56.877 | 42.799 | 99.940 | 1.00 | 68.46 | C |
| ATOM | 21 | C | PRO | A | 17 | 56.305 | 46.430 | 99.597 | 1.00 | 74.18 | C |
| ATOM | 22 | O | PRO | A | 17 | 55.252 | 47.071 | 99.514 | 1.00 | 74.07 | O |
| ATOM | 23 | N | LYS | A | 18 | 57.448 | 46.946 | 100.043 | 1.00 | 75.60 | N |
| ATOM | 24 | CA | LYS | A | 18 | 57.578 | 48.333 | 100.476 | 1.00 | 76.69 | C |
| ATOM | 25 | CB | LYS | A | 18 | 58.885 | 48.504 | 101.257 | 1.00 | 79.54 | C |
| ATOM | 26 | CG | LYS | A | 18 | 59.092 | 49.898 | 101.841 | 1.00 | 83.04 | C |
| ATOM | 27 | CD | LYS | A | 18 | 60.516 | 50.084 | 102.353 | 1.00 | 87.23 | C |
| ATOM | 28 | CE | LYS | A | 18 | 60.746 | 51.520 | 102.810 | 1.00 | 87.83 | C |
| ATOM | 29 | NZ | LYS | A | 18 | 62.185 | 51.819 | 103.030 | 1.00 | 87.02 | N |
| ATOM | 30 | C | LYS | A | 18 | 57.554 | 49.273 | 99.272 | 1.00 | 75.06 | C |
| ATOM | 31 | O | LYS | A | 18 | 56.878 | 50.303 | 99.281 | 1.00 | 71.21 | O |
| ATOM | 32 | N | TYR | A | 19 | 58.304 | 48.908 | 98.240 | 1.00 | 75.29 | N |
| ATOM | 33 | CA | TYR | A | 19 | 58.368 | 49.694 | 97.014 | 1.00 | 76.95 | C |
| ATOM | 34 | CB | TYR | A | 19 | 59.324 | 49.042 | 96.023 | 1.00 | 80.97 | C |
| ATOM | 35 | CG | TYR | A | 19 | 60.765 | 49.053 | 96.447 | 1.00 | 85.87 | C |
| ATOM | 36 | CD1 | TYR | A | 19 | 61.317 | 50.174 | 97.062 | 1.00 | 88.80 | C |
| ATOM | 37 | CE1 | TYR | A | 19 | 62.674 | 50.231 | 97.371 | 1.00 | 91.79 | C |
| ATOM | 38 | CD2 | TYR | A | 19 | 61.605 | 47.978 | 96.155 | 1.00 | 89.90 | C |
| ATOM | 39 | CE2 | TYR | A | 19 | 62.970 | 48.024 | 96.460 | 1.00 | 92.65 | C |
| ATOM | 40 | CZ | TYR | A | 19 | 63.493 | 49.153 | 97.063 | 1.00 | 92.77 | C |
| ATOM | 41 | OH | TYR | A | 19 | 64.836 | 49.217 | 97.331 | 1.00 | 92.93 | O |
| ATOM | 42 | C | TYR | A | 19 | 56.984 | 49.755 | 96.383 | 1.00 | 75.07 | C |
| ATOM | 43 | O | TYR | A | 19 | 56.493 | 50.822 | 96.034 | 1.00 | 73.10 | O |
| ATOM | 44 | N | ILE | A | 20 | 56.373 | 48.585 | 96.237 | 1.00 | 73.66 | N |
| ATOM | 45 | CA | ILE | A | 20 | 55.049 | 48.463 | 95.656 | 1.00 | 73.27 | C |
| ATOM | 46 | CB | ILE | A | 20 | 54.553 | 47.017 | 95.750 | 1.00 | 70.57 | C |
| ATOM | 47 | CG2 | ILE | A | 20 | 53.107 | 46.916 | 95.289 | 1.00 | 67.54 | C |
| ATOM | 48 | CG1 | ILE | A | 20 | 55.455 | 46.115 | 94.910 | 1.00 | 67.81 | C |
| ATOM | 49 | CD1 | ILE | A | 20 | 55.172 | 44.648 | 95.081 | 1.00 | 61.87 | C |
| ATOM | 50 | C | ILE | A | 20 | 54.051 | 49.379 | 96.356 | 1.00 | 74.61 | C |
| ATOM | 51 | O | ILE | A | 20 | 53.357 | 50.151 | 95.697 | 1.00 | 77.06 | O |
| ATOM | 52 | N | GLU | A | 21 | 53.983 | 49.302 | 97.681 | 1.00 | 74.59 | N |
| ATOM | 53 | CA | GLU | A | 21 | 53.064 | 50.132 | 98.446 | 1.00 | 74.75 | C |
| ATOM | 54 | CB | GLU | A | 21 | 53.132 | 49.748 | 99.916 | 1.00 | 78.68 | C |
| ATOM | 55 | CG | GLU | A | 21 | 52.292 | 50.626 | 100.862 | 1.00 | 84.95 | C |
| ATOM | 56 | CD | GLU | A | 21 | 50.803 | 50.606 | 100.552 | 1.00 | 90.44 | C |
| ATOM | 57 | OE1 | GLU | A | 21 | 50.224 | 49.501 | 100.467 | 1.00 | 91.32 | O |
| ATOM | 58 | OE2 | GLU | A | 21 | 50.209 | 51.697 | 100.400 | 1.00 | 92.75 | O |
| ATOM | 59 | C | GLU | A | 21 | 53.344 | 51.626 | 98.295 | 1.00 | 73.87 | C |
| ATOM | 60 | O | GLU | A | 21 | 52.428 | 52.449 | 98.355 | 1.00 | 73.69 | O |
| ATOM | 61 | N | HIS | A | 22 | 54.607 | 51.983 | 98.095 | 1.00 | 72.03 | N |
| ATOM | 62 | CA | HIS | A | 22 | 54.962 | 53.390 | 97.939 | 1.00 | 70.76 | C |
| ATOM | 63 | CB | HIS | A | 22 | 56.468 | 53.594 | 98.111 | 1.00 | 68.33 | C |
| ATOM | 64 | CG | HIS | A | 22 | 56.916 | 54.990 | 97.817 | 1.00 | 69.57 | C |
| ATOM | 65 | CD2 | HIS | A | 22 | 57.814 | 55.477 | 96.918 | 1.00 | 69.57 | C |
| ATOM | 66 | ND1 | HIS | A | 22 | 56.395 | 56.087 | 98.464 | 1.00 | 68.20 | N |
| ATOM | 67 | CE1 | HIS | A | 22 | 56.945 | 57.188 | 97.981 | 1.00 | 68.18 | C |
| ATOM | 68 | NE2 | HIS | A | 22 | 57.809 | 56.840 | 97.041 | 1.00 | 67.39 | N |
| ATOM | 69 | C | HIS | A | 22 | 54.534 | 53.968 | 96.597 | 1.00 | 71.12 | C |
| ATOM | 70 | O | HIS | A | 22 | 53.688 | 54.854 | 96.545 | 1.00 | 67.18 | O |
| ATOM | 71 | N | PHE | A | 23 | 55.128 | 53.465 | 95.517 | 1.00 | 72.51 | N |
| ATOM | 72 | CA | PHE | A | 23 | 54.810 | 53.946 | 94.185 | 1.00 | 74.41 | C |
| ATOM | 73 | CB | PHE | A | 23 | 55.718 | 53.284 | 93.151 | 1.00 | 72.27 | C |
| ATOM | 74 | CG | PHE | A | 23 | 57.164 | 53.652 | 93.295 | 1.00 | 73.33 | C |
| ATOM | 75 | CD1 | PHE | A | 23 | 58.033 | 52.849 | 94.018 | 1.00 | 74.93 | C |
| ATOM | 76 | CD2 | PHE | A | 23 | 57.656 | 54.816 | 92.721 | 1.00 | 73.74 | C |
| ATOM | 77 | CE1 | PHE | A | 23 | 59.371 | 53.199 | 94.163 | 1.00 | 75.32 | C |
| ATOM | 78 | CE2 | PHE | A | 23 | 58.994 | 55.178 | 92.860 | 1.00 | 72.26 | C |
| ATOM | 79 | CZ | PHE | A | 23 | 59.854 | 54.369 | 93.582 | 1.00 | 74.50 | C |
| ATOM | 80 | C | PHE | A | 23 | 53.343 | 53.697 | 93.853 | 1.00 | 76.10 | C |
| ATOM | 81 | O | PHE | A | 23 | 52.792 | 54.277 | 92.910 | 1.00 | 74.16 | O |
| ATOM | 82 | N | SER | A | 24 | 52.717 | 52.837 | 94.649 | 1.00 | 76.75 | N |
| ATOM | 83 | CA | SER | A | 24 | 51.311 | 52.517 | 94.490 | 1.00 | 77.02 | C |
| ATOM | 84 | CB | SER | A | 24 | 50.965 | 51.289 | 95.344 | 1.00 | 77.98 | C |
| ATOM | 85 | OG | SER | A | 24 | 49.568 | 51.069 | 95.417 | 1.00 | 82.23 | O |
| ATOM | 86 | C | SER | A | 24 | 50.479 | 53.720 | 94.943 | 1.00 | 76.98 | C |
| ATOM | 87 | O | SER | A | 24 | 49.258 | 53.719 | 94.831 | 1.00 | 76.88 | O |
| ATOM | 88 | N | LYS | A | 25 | 51.152 | 54.737 | 95.472 | 1.00 | 77.47 | N |
| ATOM | 89 | CA | LYS | A | 25 | 50.477 | 55.935 | 95.944 | 1.00 | 79.40 | C |
| ATOM | 90 | CB | LYS | A | 25 | 51.307 | 56.632 | 97.033 | 1.00 | 82.56 | C |
| ATOM | 91 | CG | LYS | A | 25 | 51.490 | 55.838 | 98.327 | 1.00 | 86.29 | C |
| ATOM | 92 | CD | LYS | A | 25 | 50.157 | 55.487 | 98.977 | 1.00 | 89.12 | C |
| ATOM | 93 | CE | LYS | A | 25 | 50.366 | 54.701 | 100.265 | 1.00 | 89.65 | C |
| ATOM | 94 | NZ | LYS | A | 25 | 49.080 | 54.190 | 100.824 | 1.00 | 93.53 | N |
| ATOM | 95 | C | LYS | A | 25 | 50.242 | 56.898 | 94.794 | 1.00 | 78.52 | C |
| ATOM | 96 | O | LYS | A | 25 | 49.277 | 57.671 | 94.812 | 1.00 | 77.09 | O |
| ATOM | 97 | N | PHE | A | 26 | 51.131 | 56.855 | 93.803 | 1.00 | 78.83 | N |
| ATOM | 98 | CA | PHE | A | 26 | 51.037 | 57.722 | 92.631 | 1.00 | 78.72 | C |
| ATOM | 99 | CB | PHE | A | 26 | 52.411 | 57.886 | 91.982 | 1.00 | 81.10 | C |
| ATOM | 100 | CG | PHE | A | 26 | 53.456 | 58.454 | 92.896 | 1.00 | 88.28 | C |
| ATOM | 101 | CD1 | PHE | A | 26 | 53.876 | 57.751 | 94.020 | 1.00 | 89.80 | C |
| ATOM | 102 | CD2 | PHE | A | 26 | 54.028 | 59.692 | 92.628 | 1.00 | 89.90 | C |
| ATOM | 103 | CE1 | PHE | A | 26 | 54.849 | 58.270 | 94.867 | 1.00 | 90.06 | C |
| ATOM | 104 | CE2 | PHE | A | 26 | 55.002 | 60.224 | 93.463 | 1.00 | 89.82 | C |
| ATOM | 105 | CZ | PHE | A | 26 | 55.414 | 59.510 | 94.587 | 1.00 | 90.59 | C |
| ATOM | 106 | C | PHE | A | 26 | 50.089 | 57.147 | 91.585 | 1.00 | 76.94 | C |
| ATOM | 107 | O | PHE | A | 26 | 49.757 | 55.958 | 91.616 | 1.00 | 74.83 | O |
| ATOM | 108 | N | SER | A | 27 | 49.649 | 58.006 | 90.668 | 1.00 | 73.78 | N |
| ATOM | 109 | CA | SER | A | 27 | 48.778 | 57.594 | 89.574 | 1.00 | 70.40 | C |
| ATOM | 110 | CB | SER | A | 27 | 47.588 | 58.535 | 89.449 | 1.00 | 68.44 | C |
| ATOM | 111 | OG | SER | A | 27 | 46.796 | 58.513 | 90.622 | 1.00 | 73.69 | O |
| ATOM | 112 | C | SER | A | 27 | 49.624 | 57.684 | 88.315 | 1.00 | 67.37 | C |
| ATOM | 113 | O | SER | A | 27 | 50.497 | 58.545 | 88.223 | 1.00 | 66.29 | O |
| ATOM | 114 | N | PRO | A | 28 | 49.405 | 56.787 | 87.338 | 1.00 | 65.31 | N |
| ATOM | 115 | CD | PRO | A | 28 | 48.592 | 55.558 | 87.322 | 1.00 | 63.63 | C |
| ATOM | 116 | CA | PRO | A | 28 | 50.228 | 56.889 | 86.128 | 1.00 | 62.52 | C |
| ATOM | 117 | CB | PRO | A | 28 | 49.824 | 55.651 | 85.316 | 1.00 | 64.73 | C |
| ATOM | 118 | CG | PRO | A | 28 | 49.367 | 54.679 | 86.367 | 1.00 | 66.10 | C |
| ATOM | 119 | C | PRO | A | 28 | 49.923 | 58.189 | 85.391 | 1.00 | 59.58 | C |
| ATOM | 120 | O | PRO | A | 28 | 48.827 | 58.719 | 85.471 | 1.00 | 56.70 | O |
| ATOM | 121 | N | SER | A | 29 | 50.905 | 58.703 | 84.669 | 1.00 | 56.77 | N |
| ATOM | 122 | CA | SER | A | 29 | 50.728 | 59.937 | 83.918 | 1.00 | 55.03 | C |
| ATOM | 123 | CB | SER | A | 29 | 51.873 | 60.913 | 84.229 | 1.00 | 54.75 | C |
| ATOM | 124 | OG | SER | A | 29 | 51.709 | 61.435 | 85.543 | 1.00 | 62.06 | O |
| ATOM | 125 | C | SER | A | 29 | 50.622 | 59.702 | 82.421 | 1.00 | 50.99 | C |
| ATOM | 126 | O | SER | A | 29 | 51.612 | 59.412 | 81.754 | 1.00 | 47.47 | O |
| ATOM | 127 | N | PRO | A | 30 | 49.402 | 59.840 | 81.876 | 1.00 | 50.73 | N |
| ATOM | 128 | CD | PRO | A | 30 | 48.197 | 60.206 | 82.637 | 1.00 | 51.68 | C |
| ATOM | 129 | CA | PRO | A | 30 | 49.074 | 59.662 | 80.457 | 1.00 | 51.75 | C |
| ATOM | 130 | CB | PRO | A | 30 | 47.554 | 59.842 | 80.416 | 1.00 | 50.07 | C |
| ATOM | 131 | CG | PRO | A | 30 | 47.101 | 59.610 | 81.803 | 1.00 | 51.80 | C |
| ATOM | 132 | C | PRO | A | 30 | 49.762 | 60.704 | 79.581 | 1.00 | 50.88 | C |
| ATOM | 133 | O | PRO | A | 30 | 49.745 | 61.895 | 79.885 | 1.00 | 52.86 | O |
| ATOM | 134 | N | LEU | A | 31 | 50.368 | 60.250 | 78.498 | 1.00 | 49.91 | N |
| ATOM | 135 | CA | LEU | A | 31 | 51.034 | 61.141 | 77.586 | 1.00 | 50.66 | C |
| ATOM | 136 | CB | LEU | A | 31 | 52.444 | 60.639 | 77.270 | 1.00 | 46.41 | C |
| ATOM | 137 | CG | LEU | A | 31 | 53.452 | 60.425 | 78.398 | 1.00 | 50.52 | C |
| ATOM | 138 | CD1 | LEU | A | 31 | 54.782 | 59.942 | 77.829 | 1.00 | 45.59 | C |
| ATOM | 139 | CD2 | LEU | A | 31 | 53.638 | 61.732 | 79.160 | 1.00 | 50.94 | C |
| ATOM | 140 | C | LEU | A | 31 | 50.215 | 61.211 | 76.293 | 1.00 | 55.07 | C |
| ATOM | 141 | O | LEU | A | 31 | 49.583 | 60.233 | 75.887 | 1.00 | 54.66 | O |
| ATOM | 142 | N | SER | A | 32 | 50.220 | 62.380 | 75.661 | 1.00 | 55.09 | N |
| ATOM | 143 | CA | SER | A | 32 | 49.507 | 62.583 | 74.412 | 1.00 | 56.13 | C |
| ATOM | 144 | CB | SER | A | 32 | 49.065 | 64.045 | 74.283 | 1.00 | 55.34 | C |
| ATOM | 145 | OG | SER | A | 32 | 50.194 | 64.908 | 74.244 | 1.00 | 52.45 | O |
| ATOM | 146 | C | SER | A | 32 | 50.461 | 62.221 | 73.275 | 1.00 | 57.51 | C |
| ATOM | 147 | O | SER | A | 32 | 51.666 | 62.138 | 73.470 | 1.00 | 54.32 | O |
| ATOM | 148 | N | MET | A | 33 | 49.922 | 62.004 | 72.085 | 1.00 | 60.09 | N |
| ATOM | 149 | CA | MET | A | 33 | 50.743 | 61.646 | 70.944 | 1.00 | 65.60 | C |
| ATOM | 150 | CB | MET | A | 33 | 49.878 | 61.421 | 69.718 | 1.00 | 68.57 | C |
| ATOM | 151 | CG | MET | A | 33 | 50.612 | 60.710 | 68.603 | 1.00 | 70.27 | C |
| ATOM | 152 | SD | MET | A | 33 | 50.488 | 58.964 | 68.852 | 1.00 | 74.61 | S |
| ATOM | 153 | CE | MET | A | 33 | 48.771 | 58.689 | 68.297 | 1.00 | 74.79 | C |
| ATOM | 154 | C | MET | A | 33 | 51.725 | 62.759 | 70.638 | 1.00 | 68.24 | C |
| ATOM | 155 | O | MET | A | 33 | 52.777 | 62.540 | 70.046 | 1.00 | 68.70 | O |
| ATOM | 156 | N | LYS | A | 34 | 51.373 | 63.966 | 71.040 | 1.00 | 70.76 | N |
| ATOM | 157 | CA | LYS | A | 34 | 52.238 | 65.096 | 70.782 | 1.00 | 73.35 | C |
| ATOM | 158 | CB | LYS | A | 34 | 51.452 | 66.397 | 70.931 | 1.00 | 78.21 | C |
| ATOM | 159 | CG | LYS | A | 34 | 51.858 | 67.453 | 69.926 | 1.00 | 84.06 | C |
| ATOM | 160 | CD | LYS | A | 34 | 50.651 | 68.088 | 69.267 | 1.00 | 87.49 | C |
| ATOM | 161 | CE | LYS | A | 34 | 50.593 | 69.571 | 69.599 | 1.00 | 90.90 | C |
| ATOM | 162 | NZ | LYS | A | 34 | 49.544 | 70.287 | 68.820 | 1.00 | 92.57 | N |
| ATOM | 163 | C | LYS | A | 34 | 53.431 | 65.078 | 71.722 | 1.00 | 72.47 | C |
| ATOM | 164 | O | LYS | A | 34 | 54.576 | 65.267 | 71.297 | 1.00 | 69.99 | O |
| ATOM | 165 | N | GLN | A | 35 | 53.164 | 64.864 | 73.006 | 1.00 | 71.23 | N |
| ATOM | 166 | CA | GLN | A | 35 | 54.241 | 64.789 | 73.985 | 1.00 | 70.20 | C |
| ATOM | 167 | CB | GLN | A | 35 | 53.683 | 64.455 | 75.370 | 1.00 | 67.16 | C |
| ATOM | 168 | CG | GLN | A | 35 | 52.744 | 65.503 | 75.903 | 1.00 | 68.75 | C |
| ATOM | 169 | CD | GLN | A | 35 | 52.148 | 65.146 | 77.242 | 1.00 | 69.50 | C |
| ATOM | 170 | OE1 | GLN | A | 35 | 51.246 | 64.324 | 77.335 | 1.00 | 73.21 | O |
| ATOM | 171 | NE2 | GLN | A | 35 | 52.655 | 65.764 | 78.289 | 1.00 | 68.60 | N |
| ATOM | 172 | C | GLN | A | 35 | 55.221 | 63.710 | 73.535 | 1.00 | 71.61 | C |
| ATOM | 173 | O | GLN | A | 35 | 56.432 | 63.933 | 73.566 | 1.00 | 71.53 | O |
| ATOM | 174 | N | PHE | A | 36 | 54.689 | 62.555 | 73.111 | 1.00 | 71.79 | N |
| ATOM | 175 | CA | PHE | A | 36 | 55.512 | 61.444 | 72.626 | 1.00 | 71.37 | C |
| ATOM | 176 | CB | PHE | A | 36 | 54.643 | 60.294 | 72.107 | 1.00 | 68.87 | C |
| ATOM | 177 | CG | PHE | A | 36 | 54.226 | 59.317 | 73.165 | 1.00 | 68.27 | C |
| ATOM | 178 | CD1 | PHE | A | 36 | 52.900 | 59.276 | 73.601 | 1.00 | 64.95 | C |
| ATOM | 179 | CD2 | PHE | A | 36 | 55.153 | 58.430 | 73.723 | 1.00 | 65.45 | C |
| ATOM | 180 | CE1 | PHE | A | 36 | 52.498 | 58.368 | 74.575 | 1.00 | 65.35 | C |
| ATOM | 181 | CE2 | PHE | A | 36 | 54.766 | 57.524 | 74.690 | 1.00 | 65.38 | C |
| ATOM | 182 | CZ | PHE | A | 36 | 53.435 | 57.489 | 75.120 | 1.00 | 65.74 | C |
| ATOM | 183 | C | PHE | A | 36 | 56.400 | 61.916 | 71.488 | 1.00 | 72.76 | C |
| ATOM | 184 | O | PHE | A | 36 | 57.577 | 61.578 | 71.406 | 1.00 | 67.55 | O |
| ATOM | 185 | N | LEU | A | 37 | 55.814 | 62.697 | 70.591 | 1.00 | 76.65 | N |
| ATOM | 186 | CA | LEU | A | 37 | 56.558 | 63.207 | 69.451 | 1.00 | 81.89 | C |
| ATOM | 187 | CB | LEU | A | 37 | 55.607 | 63.844 | 68.424 | 1.00 | 80.04 | C |
| ATOM | 188 | CG | LEU | A | 37 | 55.072 | 62.851 | 67.392 | 1.00 | 79.28 | C |
| ATOM | 189 | CD1 | LEU | A | 37 | 53.977 | 63.512 | 66.577 | 1.00 | 80.31 | C |
| ATOM | 190 | CD2 | LEU | A | 37 | 56.208 | 62.379 | 66.494 | 1.00 | 77.17 | C |
| ATOM | 191 | C | LEU | A | 37 | 57.646 | 64.193 | 69.864 | 1.00 | 85.64 | C |
| ATOM | 192 | O | LEU | A | 37 | 58.750 | 64.147 | 69.321 | 1.00 | 86.77 | O |
| ATOM | 193 | N | ASP | A | 38 | 57.349 | 65.072 | 70.818 | 1.00 | 89.45 | N |
| ATOM | 194 | CA | ASP | A | 38 | 58.357 | 66.020 | 71.254 | 1.00 | 94.54 | C |
| ATOM | 195 | CB | ASP | A | 38 | 57.729 | 67.224 | 71.956 | 1.00 | 97.61 | C |
| ATOM | 196 | CG | ASP | A | 38 | 57.152 | 68.236 | 70.982 | 1.00 | 102.16 | C |
| ATOM | 197 | OD1 | ASP | A | 38 | 57.626 | 68.287 | 69.822 | 1.00 | 104.24 | O |
| ATOM | 198 | OD2 | ASP | A | 38 | 56.240 | 69.009 | 71.380 | 1.00 | 103.41 | O |
| ATOM | 199 | C | ASP | A | 38 | 59.388 | 65.377 | 72.174 | 1.00 | 95.35 | C |
| ATOM | 200 | O | ASP | A | 38 | 60.500 | 65.882 | 72.321 | 1.00 | 95.23 | O |
| ATOM | 201 | N | PHE | A | 39 | 59.027 | 64.248 | 72.772 | 1.00 | 97.16 | N |
| ATOM | 202 | CA | PHE | A | 39 | 59.929 | 63.536 | 73.667 | 1.00 | 98.83 | C |
| ATOM | 203 | CB | PHE | A | 39 | 59.203 | 62.391 | 74.359 | 1.00 | 98.36 | C |
| ATOM | 204 | CG | PHE | A | 39 | 59.322 | 62.426 | 75.849 | 1.00 | 97.41 | C |
| ATOM | 205 | CD1 | PHE | A | 39 | 60.565 | 62.485 | 76.458 | 1.00 | 96.65 | C |
| ATOM | 206 | CD2 | PHE | A | 39 | 58.188 | 62.425 | 76.644 | 1.00 | 97.95 | C |
| ATOM | 207 | CE1 | PHE | A | 39 | 60.679 | 62.546 | 77.841 | 1.00 | 97.23 | C |
| ATOM | 208 | CE2 | PHE | A | 39 | 58.283 | 62.485 | 78.028 | 1.00 | 98.27 | C |
| ATOM | 209 | CZ | PHE | A | 39 | 59.534 | 62.545 | 78.633 | 1.00 | 97.77 | C |
| ATOM | 210 | C | PHE | A | 39 | 61.117 | 62.975 | 72.908 | 1.00 | 101.54 | C |
| ATOM | 211 | O | PHE | A | 39 | 62.224 | 62.930 | 73.429 | 1.00 | 102.53 | O |
| ATOM | 212 | N | GLY | A | 40 | 60.881 | 62.536 | 71.677 | 1.00 | 105.04 | N |
| ATOM | 213 | CA | GLY | A | 40 | 61.953 | 61.988 | 70.864 | 1.00 | 110.50 | C |
| ATOM | 214 | C | GLY | A | 40 | 62.514 | 63.035 | 69.922 | 1.00 | 114.56 | C |
| ATOM | 215 | O | GLY | A | 40 | 63.648 | 62.952 | 69.473 | 1.00 | 114.86 | O |
| ATOM | 216 | N | SER | A | 41 | 61.703 | 64.041 | 69.634 | 1.00 | 118.73 | N |
| ATOM | 217 | CA | SER | A | 41 | 62.086 | 65.123 | 68.738 | 1.00 | 123.56 | C |
| ATOM | 218 | CB | SER | A | 41 | 60.850 | 65.955 | 68.370 | 1.00 | 125.27 | C |
| ATOM | 219 | OG | SER | A | 41 | 61.127 | 66.863 | 67.309 | 1.00 | 128.06 | O |
| ATOM | 220 | C | SER | A | 41 | 63.153 | 66.060 | 69.295 | 1.00 | 125.34 | C |
| ATOM | 221 | O | SER | A | 41 | 64.352 | 65.776 | 69.233 | 1.00 | 124.74 | O |
| ATOM | 222 | N | SER | A | 42 | 62.682 | 67.186 | 69.829 | 1.00 | 127.77 | N |
| ATOM | 223 | CA | SER | A | 42 | 63.530 | 68.244 | 70.376 | 1.00 | 130.32 | C |
| ATOM | 224 | CB | SER | A | 42 | 62.799 | 69.589 | 70.261 | 1.00 | 130.42 | C |
| ATOM | 225 | OG | SER | A | 42 | 63.628 | 70.662 | 70.680 | 1.00 | 129.86 | O |
| ATOM | 226 | C | SER | A | 42 | 64.025 | 68.060 | 71.816 | 1.00 | 131.15 | C |
| ATOM | 227 | O | SER | A | 42 | 65.232 | 68.004 | 72.052 | 1.00 | 132.09 | O |
| ATOM | 228 | N | ASN | A | 43 | 63.104 | 67.985 | 72.775 | 1.00 | 130.97 | N |
| ATOM | 229 | CA | ASN | A | 43 | 63.487 | 67.818 | 74.175 | 1.00 | 129.81 | C |
| ATOM | 230 | CB | ASN | A | 43 | 62.260 | 67.515 | 75.038 | 1.00 | 130.90 | C |
| ATOM | 231 | CG | ASN | A | 43 | 62.519 | 67.735 | 76.524 | 1.00 | 131.71 | C |
| ATOM | 232 | OD1 | ASN | A | 43 | 63.645 | 67.593 | 76.996 | 1.00 | 130.91 | O |
| ATOM | 233 | ND2 | ASN | A | 43 | 61.467 | 68.070 | 77.267 | 1.00 | 131.67 | N |
| ATOM | 234 | C | ASN | A | 43 | 64.446 | 66.643 | 74.267 | 1.00 | 128.06 | C |
| ATOM | 235 | O | ASN | A | 43 | 65.409 | 66.652 | 75.037 | 1.00 | 127.91 | O |
| ATOM | 236 | N | ALA | A | 44 | 64.162 | 65.643 | 73.444 | 1.00 | 126.29 | N |
| ATOM | 237 | CA | ALA | A | 44 | 64.922 | 64.408 | 73.364 | 1.00 | 124.85 | C |
| ATOM | 238 | CB | ALA | A | 44 | 64.778 | 63.823 | 71.977 | 1.00 | 124.77 | C |
| ATOM | 239 | C | ALA | A | 44 | 66.391 | 64.461 | 73.747 | 1.00 | 123.45 | C |
| ATOM | 240 | O | ALA | A | 44 | 67.249 | 64.945 | 73.001 | 1.00 | 123.25 | O |
| ATOM | 241 | N | CYS | A | 45 | 66.638 | 63.943 | 74.938 | 1.00 | 121.07 | N |
| ATOM | 242 | CA | CYS | A | 45 | 67.946 | 63.817 | 75.546 | 1.00 | 118.82 | C |
| ATOM | 243 | CB | CYS | A | 45 | 68.379 | 65.114 | 76.234 | 1.00 | 120.61 | C |
| ATOM | 244 | SG | CYS | A | 45 | 69.403 | 66.184 | 75.208 | 1.00 | 125.59 | S |
| ATOM | 245 | C | CYS | A | 45 | 67.612 | 62.763 | 76.571 | 1.00 | 115.26 | C |
| ATOM | 246 | O | CYS | A | 45 | 66.931 | 63.050 | 77.548 | 1.00 | 114.95 | O |
| ATOM | 247 | N | GLU | A | 46 | 68.049 | 61.535 | 76.316 | 1.00 | 110.67 | N |
| ATOM | 248 | CA | GLU | A | 46 | 67.765 | 60.428 | 77.207 | 1.00 | 106.78 | C |
| ATOM | 249 | CB | GLU | A | 46 | 68.777 | 59.305 | 77.011 | 1.00 | 105.89 | C |
| ATOM | 250 | CG | GLU | A | 46 | 69.838 | 59.592 | 75.982 | 1.00 | 104.16 | C |
| ATOM | 251 | CD | GLU | A | 46 | 70.737 | 58.402 | 75.765 | 1.00 | 105.09 | C |
| ATOM | 252 | OE1 | GLU | A | 46 | 70.608 | 57.421 | 76.522 | 1.00 | 103.81 | O |
| ATOM | 253 | OE2 | GLU | A | 46 | 71.576 | 58.442 | 74.846 | 1.00 | 107.47 | O |
| ATOM | 254 | C | GLU | A | 46 | 67.734 | 60.835 | 78.663 | 1.00 | 104.80 | C |
| ATOM | 255 | O | GLU | A | 46 | 67.118 | 60.160 | 79.478 | 1.00 | 106.03 | O |
| ATOM | 256 | N | LYS | A | 47 | 68.404 | 61.931 | 78.995 | 1.00 | 101.54 | N |
| ATOM | 257 | CA | LYS | A | 47 | 68.417 | 62.413 | 80.368 | 1.00 | 98.40 | C |
| ATOM | 258 | CB | LYS | A | 47 | 69.314 | 63.643 | 80.485 | 1.00 | 100.84 | C |
| ATOM | 259 | CG | LYS | A | 47 | 70.757 | 63.378 | 80.126 | 1.00 | 102.41 | C |
| ATOM | 260 | CD | LYS | A | 47 | 71.616 | 64.590 | 80.440 | 1.00 | 105.64 | C |
| ATOM | 261 | CE | LYS | A | 47 | 73.086 | 64.296 | 80.195 | 1.00 | 106.69 | C |
| ATOM | 262 | NZ | LYS | A | 47 | 73.934 | 65.465 | 80.537 | 1.00 | 106.11 | N |
| ATOM | 263 | C | LYS | A | 47 | 66.995 | 62.772 | 80.773 | 1.00 | 94.73 | C |
| ATOM | 264 | O | LYS | A | 47 | 66.535 | 62.418 | 81.861 | 1.00 | 93.34 | O |
| ATOM | 265 | N | THR | A | 48 | 66.308 | 63.478 | 79.879 | 1.00 | 91.50 | N |
| ATOM | 266 | CA | THR | A | 48 | 64.923 | 63.902 | 80.088 | 1.00 | 88.54 | C |
| ATOM | 267 | CB | THR | A | 48 | 64.448 | 64.793 | 78.928 | 1.00 | 89.70 | C |
| ATOM | 268 | OG1 | THR | A | 48 | 65.366 | 65.880 | 78.751 | 1.00 | 91.21 | O |
| ATOM | 269 | CG2 | THR | A | 48 | 63.063 | 65.340 | 79.214 | 1.00 | 89.16 | C |
| ATOM | 270 | C | THR | A | 48 | 64.009 | 62.675 | 80.160 | 1.00 | 85.30 | C |
| ATOM | 271 | O | THR | A | 48 | 63.114 | 62.595 | 81.001 | 1.00 | 82.67 | O |
| ATOM | 272 | N | SER | A | 49 | 64.251 | 61.720 | 79.266 | 1.00 | 81.38 | N |
| ATOM | 273 | CA | SER | A | 49 | 63.483 | 60.485 | 79.225 | 1.00 | 79.44 | C |
| ATOM | 274 | CB | SER | A | 49 | 63.784 | 59.735 | 77.930 | 1.00 | 78.56 | C |
| ATOM | 275 | OG | SER | A | 49 | 63.026 | 58.545 | 77.839 | 1.00 | 80.48 | O |
| ATOM | 276 | C | SER | A | 49 | 63.857 | 59.626 | 80.437 | 1.00 | 78.68 | C |
| ATOM | 277 | O | SER | A | 49 | 62.978 | 59.116 | 81.136 | 1.00 | 79.74 | O |
| ATOM | 278 | N | PHE | A | 50 | 65.159 | 59.482 | 80.680 | 1.00 | 75.83 | N |
| ATOM | 279 | CA | PHE | A | 50 | 65.678 | 58.711 | 81.805 | 1.00 | 73.90 | C |
| ATOM | 280 | CB | PHE | A | 50 | 67.198 | 58.868 | 81.886 | 1.00 | 74.92 | C |
| ATOM | 281 | CG | PHE | A | 50 | 67.798 | 58.332 | 83.152 | 1.00 | 79.21 | C |
| ATOM | 282 | CD1 | PHE | A | 50 | 67.892 | 56.955 | 83.367 | 1.00 | 79.03 | C |
| ATOM | 283 | CD2 | PHE | A | 50 | 68.250 | 59.202 | 84.146 | 1.00 | 78.15 | C |
| ATOM | 284 | CE1 | PHE | A | 50 | 68.428 | 56.449 | 84.556 | 1.00 | 78.85 | C |
| ATOM | 285 | CE2 | PHE | A | 50 | 68.787 | 58.706 | 85.337 | 1.00 | 80.53 | C |
| ATOM | 286 | CZ | PHE | A | 50 | 68.876 | 57.327 | 85.540 | 1.00 | 80.17 | C |
| ATOM | 287 | C | PHE | A | 50 | 65.053 | 59.144 | 83.127 | 1.00 | 73.22 | C |
| ATOM | 288 | O | PHE | A | 50 | 64.585 | 58.311 | 83.900 | 1.00 | 72.46 | O |
| ATOM | 289 | N | THR | A | 51 | 65.059 | 60.448 | 83.383 | 1.00 | 70.81 | N |
| ATOM | 290 | CA | THR | A | 51 | 64.503 | 60.998 | 84.617 | 1.00 | 70.77 | C |
| ATOM | 291 | CB | THR | A | 51 | 64.755 | 62.516 | 84.738 | 1.00 | 73.34 | C |
| ATOM | 292 | OG1 | THR | A | 51 | 66.155 | 62.797 | 84.612 | 1.00 | 75.73 | O |
| ATOM | 293 | CG2 | THR | A | 51 | 64.262 | 63.023 | 86.093 | 1.00 | 72.90 | C |
| ATOM | 294 | C | THR | A | 51 | 62.998 | 60.794 | 84.678 | 1.00 | 67.81 | C |
| ATOM | 295 | O | THR | A | 51 | 62.416 | 60.696 | 85.755 | 1.00 | 65.97 | O |
| ATOM | 296 | N | PHE | A | 52 | 62.366 | 60.747 | 83.512 | 1.00 | 65.31 | N |
| ATOM | 297 | CA | PHE | A | 52 | 60.930 | 60.541 | 83.458 | 1.00 | 61.21 | C |
| ATOM | 298 | CB | PHE | A | 52 | 60.387 | 60.918 | 82.071 | 1.00 | 63.56 | C |
| ATOM | 299 | CG | PHE | A | 52 | 58.937 | 60.554 | 81.860 | 1.00 | 64.76 | C |
| ATOM | 300 | CD1 | PHE | A | 52 | 57.929 | 61.200 | 82.573 | 1.00 | 62.08 | C |
| ATOM | 301 | CD2 | PHE | A | 52 | 58.584 | 59.534 | 80.973 | 1.00 | 66.71 | C |
| ATOM | 302 | CE1 | PHE | A | 52 | 56.585 | 60.835 | 82.411 | 1.00 | 65.49 | C |
| ATOM | 303 | CE2 | PHE | A | 52 | 57.246 | 59.160 | 80.801 | 1.00 | 67.02 | C |
| ATOM | 304 | CZ | PHE | A | 52 | 56.246 | 59.809 | 81.522 | 1.00 | 67.60 | C |
| ATOM | 305 | C | PHE | A | 52 | 60.602 | 59.078 | 83.759 | 1.00 | 58.84 | C |
| ATOM | 306 | O | PHE | A | 52 | 59.851 | 58.782 | 84.697 | 1.00 | 52.40 | O |
| ATOM | 307 | N | LEU | A | 53 | 61.177 | 58.173 | 82.970 | 1.00 | 59.38 | N |
| ATOM | 308 | CA | LEU | A | 53 | 60.917 | 56.740 | 83.106 | 1.00 | 64.10 | C |
| ATOM | 309 | CB | LEU | A | 53 | 61.597 | 55.998 | 81.964 | 1.00 | 61.54 | C |
| ATOM | 310 | CG | LEU | A | 53 | 61.095 | 56.419 | 80.574 | 1.00 | 62.86 | C |
| ATOM | 311 | CD1 | LEU | A | 53 | 61.882 | 55.686 | 79.498 | 1.00 | 62.29 | C |
| ATOM | 312 | CD2 | LEU | A | 53 | 59.608 | 56.137 | 80.460 | 1.00 | 58.72 | C |
| ATOM | 313 | C | LEU | A | 53 | 61.291 | 56.095 | 84.433 | 1.00 | 67.23 | C |
| ATOM | 314 | O | LEU | A | 53 | 60.681 | 55.096 | 84.821 | 1.00 | 68.13 | O |
| ATOM | 315 | N | ARG | A | 54 | 62.284 | 56.662 | 85.120 | 1.00 | 70.58 | N |
| ATOM | 316 | CA | ARG | A | 54 | 62.748 | 56.163 | 86.422 | 1.00 | 72.02 | C |
| ATOM | 317 | CB | ARG | A | 54 | 63.873 | 57.027 | 86.961 | 1.00 | 78.41 | C |
| ATOM | 318 | CG | ARG | A | 54 | 65.196 | 56.884 | 86.287 | 1.00 | 85.26 | C |
| ATOM | 319 | CD | ARG | A | 54 | 66.062 | 58.062 | 86.694 | 1.00 | 92.89 | C |
| ATOM | 320 | NE | ARG | A | 54 | 65.880 | 58.417 | 88.099 | 1.00 | 98.38 | N |
| ATOM | 321 | CZ | ARG | A | 54 | 66.636 | 59.293 | 88.755 | 1.00 | 99.90 | C |
| ATOM | 322 | NH1 | ARG | A | 54 | 67.634 | 59.907 | 88.134 | 1.00 | 99.22 | N |
| ATOM | 323 | NH2 | ARG | A | 54 | 66.390 | 59.560 | 90.032 | 1.00 | 101.26 | N |
| ATOM | 324 | C | ARG | A | 54 | 61.642 | 56.202 | 87.450 | 1.00 | 71.16 | C |
| ATOM | 325 | O | ARG | A | 54 | 61.599 | 55.372 | 88.351 | 1.00 | 72.17 | O |
| ATOM | 326 | N | GLN | A | 55 | 60.774 | 57.199 | 87.334 | 1.00 | 68.43 | N |
| ATOM | 327 | CA | GLN | A | 55 | 59.667 | 57.325 | 88.258 | 1.00 | 68.06 | C |
| ATOM | 328 | CB | GLN | A | 55 | 59.494 | 58.790 | 88.675 | 1.00 | 70.13 | C |
| ATOM | 329 | CG | GLN | A | 55 | 59.659 | 59.029 | 90.174 | 1.00 | 71.98 | C |
| ATOM | 330 | CD | GLN | A | 55 | 58.340 | 59.024 | 90.921 | 1.00 | 76.37 | C |
| ATOM | 331 | OE1 | GLN | A | 55 | 58.272 | 58.619 | 92.080 | 1.00 | 76.37 | O |
| ATOM | 332 | NE2 | GLN | A | 55 | 57.285 | 59.497 | 90.266 | 1.00 | 78.25 | N |
| ATOM | 333 | C | GLN | A | 55 | 58.376 | 56.782 | 87.646 | 1.00 | 64.41 | C |
| ATOM | 334 | O | GLN | A | 55 | 57.597 | 56.129 | 88.338 | 1.00 | 63.99 | O |
| ATOM | 335 | N | GLU | A | 56 | 58.166 | 57.028 | 86.354 | 1.00 | 59.52 | N |
| ATOM | 336 | CA | GLU | A | 56 | 56.943 | 56.575 | 85.698 | 1.00 | 57.49 | C |
| ATOM | 337 | CB | GLU | A | 56 | 56.851 | 57.125 | 84.270 | 1.00 | 55.36 | C |
| ATOM | 338 | CG | GLU | A | 56 | 55.436 | 57.059 | 83.687 | 1.00 | 50.27 | C |
| ATOM | 339 | CD | GLU | A | 56 | 54.387 | 57.573 | 84.655 | 1.00 | 53.01 | C |
| ATOM | 340 | OE1 | GLU | A | 56 | 54.738 | 58.412 | 85.513 | 1.00 | 51.37 | O |
| ATOM | 341 | OE2 | GLU | A | 56 | 53.217 | 57.145 | 84.564 | 1.00 | 48.59 | O |
| ATOM | 342 | C | GLU | A | 56 | 56.778 | 55.060 | 85.667 | 1.00 | 54.33 | C |
| ATOM | 343 | O | GLU | A | 56 | 55.764 | 54.531 | 86.109 | 1.00 | 45.88 | O |
| ATOM | 344 | N | LEU | A | 57 | 57.781 | 54.366 | 85.134 | 1.00 | 51.01 | N |
| ATOM | 345 | CA | LEU | A | 57 | 57.744 | 52.901 | 85.052 | 1.00 | 51.75 | C |
| ATOM | 346 | CB | LEU | A | 57 | 59.069 | 52.351 | 84.497 | 1.00 | 49.36 | C |
| ATOM | 347 | CG | LEU | A | 57 | 59.248 | 52.650 | 82.997 | 1.00 | 53.41 | C |
| ATOM | 348 | CD1 | LEU | A | 57 | 60.622 | 52.213 | 82.520 | 1.00 | 49.67 | C |
| ATOM | 349 | CD2 | LEU | A | 57 | 58.146 | 51.957 | 82.198 | 1.00 | 52.67 | C |
| ATOM | 350 | C | LEU | A | 57 | 57.435 | 52.266 | 86.411 | 1.00 | 52.88 | C |
| ATOM | 351 | O | LEU | A | 57 | 56.561 | 51.403 | 86.514 | 1.00 | 55.18 | O |
| ATOM | 352 | N | PRO | A | 58 | 58.134 | 52.696 | 87.477 | 1.00 | 55.48 | N |
| ATOM | 353 | CD | PRO | A | 58 | 59.400 | 53.453 | 87.507 | 1.00 | 55.69 | C |
| ATOM | 354 | CA | PRO | A | 58 | 57.859 | 52.113 | 88.790 | 1.00 | 56.67 | C |
| ATOM | 355 | CB | PRO | A | 58 | 58.905 | 52.770 | 89.672 | 1.00 | 56.48 | C |
| ATOM | 356 | CG | PRO | A | 58 | 60.072 | 52.903 | 88.747 | 1.00 | 56.55 | C |
| ATOM | 357 | C | PRO | A | 58 | 56.431 | 52.385 | 89.263 | 1.00 | 58.10 | C |
| ATOM | 358 | O | PRO | A | 58 | 55.870 | 51.601 | 90.036 | 1.00 | 58.19 | O |
| ATOM | 359 | N | VAL | A | 59 | 55.846 | 53.487 | 88.800 | 1.00 | 57.45 | N |
| ATOM | 360 | CA | VAL | A | 59 | 54.481 | 53.817 | 89.174 | 1.00 | 56.00 | C |
| ATOM | 361 | CB | VAL | A | 59 | 54.143 | 55.301 | 88.866 | 1.00 | 58.58 | C |
| ATOM | 362 | CG1 | VAL | A | 59 | 52.719 | 55.639 | 89.368 | 1.00 | 55.92 | C |
| ATOM | 363 | CG2 | VAL | A | 59 | 55.155 | 56.207 | 89.535 | 1.00 | 58.78 | C |
| ATOM | 364 | C | VAL | A | 59 | 53.499 | 52.920 | 88.421 | 1.00 | 53.07 | C |
| ATOM | 365 | O | VAL | A | 59 | 52.489 | 52.477 | 88.977 | 1.00 | 51.17 | O |
| ATOM | 366 | N | ARG | A | 60 | 53.802 | 52.650 | 87.157 | 1.00 | 51.94 | N |
| ATOM | 367 | CA | ARG | A | 60 | 52.936 | 51.808 | 86.344 | 1.00 | 54.47 | C |
| ATOM | 368 | CB | ARG | A | 60 | 53.250 | 52.000 | 84.858 | 1.00 | 51.00 | C |
| ATOM | 369 | CG | ARG | A | 60 | 52.940 | 53.404 | 84.395 | 1.00 | 50.79 | C |
| ATOM | 370 | CD | ARG | A | 60 | 52.924 | 53.527 | 82.883 | 1.00 | 49.83 | C |
| ATOM | 371 | NE | ARG | A | 60 | 52.709 | 54.898 | 82.463 | 1.00 | 47.47 | N |
| ATOM | 372 | CZ | ARG | A | 60 | 52.611 | 55.289 | 81.199 | 1.00 | 50.75 | C |
| ATOM | 373 | NH1 | ARG | A | 60 | 52.696 | 54.415 | 80.206 | 1.00 | 50.14 | N |
| ATOM | 374 | NH2 | ARG | A | 60 | 52.462 | 56.572 | 80.930 | 1.00 | 48.78 | N |
| ATOM | 375 | C | ARG | A | 60 | 53.034 | 50.342 | 86.737 | 1.00 | 54.21 | C |
| ATOM | 376 | O | ARG | A | 60 | 52.025 | 49.642 | 86.787 | 1.00 | 51.91 | O |
| ATOM | 377 | N | LEU | A | 61 | 54.251 | 49.892 | 87.038 | 1.00 | 56.17 | N |
| ATOM | 378 | CA | LEU | A | 61 | 54.492 | 48.512 | 87.464 | 1.00 | 56.22 | C |
| ATOM | 379 | CB | LEU | A | 61 | 55.989 | 48.265 | 87.606 | 1.00 | 55.54 | C |
| ATOM | 380 | CG | LEU | A | 61 | 56.771 | 48.260 | 86.292 | 1.00 | 57.03 | C |
| ATOM | 381 | CD1 | LEU | A | 61 | 58.250 | 48.199 | 86.590 | 1.00 | 54.22 | C |
| ATOM | 382 | CD2 | LEU | A | 61 | 56.317 | 47.073 | 85.441 | 1.00 | 56.83 | C |
| ATOM | 383 | C | LEU | A | 61 | 53.819 | 48.241 | 88.801 | 1.00 | 56.92 | C |
| ATOM | 384 | O | LEU | A | 61 | 53.142 | 47.230 | 88.978 | 1.00 | 54.92 | O |
| ATOM | 385 | N | ALA | A | 62 | 54.019 | 49.166 | 89.733 | 1.00 | 57.36 | N |
| ATOM | 386 | CA | ALA | A | 62 | 53.452 | 49.074 | 91.065 | 1.00 | 59.06 | C |
| ATOM | 387 | CB | ALA | A | 62 | 53.934 | 50.241 | 91.912 | 1.00 | 58.62 | C |
| ATOM | 388 | C | ALA | A | 62 | 51.928 | 49.039 | 91.039 | 1.00 | 59.56 | C |
| ATOM | 389 | O | ALA | A | 62 | 51.310 | 48.253 | 91.770 | 1.00 | 58.43 | O |
| ATOM | 390 | N | ASN | A | 63 | 51.326 | 49.874 | 90.193 | 1.00 | 61.27 | N |
| ATOM | 391 | CA | ASN | A | 63 | 49.875 | 49.926 | 90.103 | 1.00 | 62.39 | C |
| ATOM | 392 | CB | ASN | A | 63 | 49.411 | 51.089 | 89.215 | 1.00 | 66.53 | C |
| ATOM | 393 | CG | ASN | A | 63 | 49.241 | 52.398 | 89.996 | 1.00 | 71.27 | C |
| ATOM | 394 | OD1 | ASN | A | 63 | 50.218 | 53.067 | 90.337 | 1.00 | 71.49 | O |
| ATOM | 395 | ND2 | ASN | A | 63 | 47.995 | 52.750 | 90.298 | 1.00 | 71.26 | N |
| ATOM | 396 | C | ASN | A | 63 | 49.267 | 48.625 | 89.609 | 1.00 | 60.33 | C |
| ATOM | 397 | O | ASN | A | 63 | 48.248 | 48.176 | 90.156 | 1.00 | 56.02 | O |
| ATOM | 398 | N | ILE | A | 64 | 49.872 | 48.006 | 88.596 | 1.00 | 59.43 | N |
| ATOM | 399 | CA | ILE | A | 64 | 49.321 | 46.745 | 88.117 | 1.00 | 63.63 | C |
| ATOM | 400 | CB | ILE | A | 64 | 49.843 | 46.390 | 86.700 | 1.00 | 62.03 | C |
| ATOM | 401 | CG2 | ILE | A | 64 | 51.277 | 45.904 | 86.752 | 1.00 | 60.16 | C |
| ATOM | 402 | CG1 | ILE | A | 64 | 48.940 | 45.314 | 86.081 | 1.00 | 64.02 | C |
| ATOM | 403 | CD1 | ILE | A | 64 | 47.441 | 45.630 | 86.161 | 1.00 | 58.22 | C |
| ATOM | 404 | C | ILE | A | 64 | 49.606 | 45.604 | 89.111 | 1.00 | 66.58 | C |
| ATOM | 405 | O | ILE | A | 64 | 48.780 | 44.700 | 89.288 | 1.00 | 65.58 | O |
| ATOM | 406 | N | MET | A | 65 | 50.757 | 45.672 | 89.780 | 1.00 | 67.98 | N |
| ATOM | 407 | CA | MET | A | 65 | 51.145 | 44.669 | 90.771 | 1.00 | 69.55 | C |
| ATOM | 408 | CB | MET | A | 65 | 52.616 | 44.842 | 91.165 | 1.00 | 67.60 | C |
| ATOM | 409 | CG | MET | A | 65 | 53.597 | 44.433 | 90.081 | 1.00 | 73.66 | C |
| ATOM | 410 | SD | MET | A | 65 | 55.294 | 44.952 | 90.420 | 1.00 | 75.96 | S |
| ATOM | 411 | CE | MET | A | 65 | 55.697 | 43.846 | 91.775 | 1.00 | 78.62 | C |
| ATOM | 412 | C | MET | A | 65 | 50.271 | 44.760 | 92.014 | 1.00 | 70.87 | C |
| ATOM | 413 | O | MET | A | 65 | 49.985 | 43.748 | 92.649 | 1.00 | 69.67 | O |
| ATOM | 414 | N | LYS | A | 66 | 49.854 | 45.976 | 92.355 | 1.00 | 72.97 | N |
| ATOM | 415 | CA | LYS | A | 66 | 49.004 | 46.202 | 93.518 | 1.00 | 76.90 | C |
| ATOM | 416 | CB | LYS | A | 66 | 48.794 | 47.707 | 93.733 | 1.00 | 77.14 | C |
| ATOM | 417 | CG | LYS | A | 66 | 48.862 | 48.176 | 95.178 | 1.00 | 78.64 | C |
| ATOM | 418 | CD | LYS | A | 66 | 47.699 | 47.694 | 96.028 | 1.00 | 79.68 | C |
| ATOM | 419 | CE | LYS | A | 66 | 47.816 | 48.259 | 97.446 | 1.00 | 79.66 | C |
| ATOM | 420 | NZ | LYS | A | 66 | 46.655 | 47.893 | 98.321 | 1.00 | 81.81 | N |
| ATOM | 421 | C | LYS | A | 66 | 47.658 | 45.525 | 93.253 | 1.00 | 78.90 | C |
| ATOM | 422 | O | LYS | A | 66 | 47.053 | 44.935 | 94.152 | 1.00 | 79.50 | O |
| ATOM | 423 | N | GLU | A | 67 | 47.206 | 45.615 | 92.006 | 1.00 | 80.85 | N |
| ATOM | 424 | CA | GLU | A | 67 | 45.939 | 45.033 | 91.590 | 1.00 | 84.67 | C |
| ATOM | 425 | CB | GLU | A | 67 | 45.593 | 45.517 | 90.185 | 1.00 | 87.54 | C |
| ATOM | 426 | CG | GLU | A | 67 | 44.163 | 45.265 | 89.797 | 1.00 | 93.64 | C |
| ATOM | 427 | CD | GLU | A | 67 | 43.700 | 46.173 | 88.687 | 1.00 | 97.86 | C |
| ATOM | 428 | OE1 | GLU | A | 67 | 44.426 | 47.139 | 88.368 | 1.00 | 98.86 | O |
| ATOM | 429 | OE2 | GLU | A | 67 | 42.601 | 45.928 | 88.144 | 1.00 | 101.27 | O |
| ATOM | 430 | C | GLU | A | 67 | 45.975 | 43.509 | 91.621 | 1.00 | 84.79 | C |
| ATOM | 431 | O | GLU | A | 67 | 45.031 | 42.852 | 92.087 | 1.00 | 84.01 | O |
| ATOM | 432 | N | ILE | A | 68 | 47.061 | 42.948 | 91.107 | 1.00 | 83.32 | N |
| ATOM | 433 | CA | ILE | A | 68 | 47.234 | 41.504 | 91.101 | 1.00 | 82.50 | C |
| ATOM | 434 | CB | ILE | A | 68 | 48.616 | 41.129 | 90.511 | 1.00 | 78.37 | C |
| ATOM | 435 | CG2 | ILE | A | 68 | 49.009 | 39.733 | 90.934 | 1.00 | 76.73 | C |
| ATOM | 436 | CG1 | ILE | A | 68 | 48.571 | 41.269 | 88.989 | 1.00 | 74.33 | C |
| ATOM | 437 | CD1 | ILE | A | 68 | 49.908 | 41.144 | 88.319 | 1.00 | 69.70 | C |
| ATOM | 438 | C | ILE | A | 68 | 47.115 | 40.942 | 92.526 | 1.00 | 84.64 | C |
| ATOM | 439 | O | ILE | A | 68 | 46.321 | 40.034 | 92.782 | 1.00 | 84.91 | O |
| ATOM | 440 | N | ASN | A | 69 | 47.901 | 41.500 | 93.448 | 1.00 | 86.04 | N |
| ATOM | 441 | CA | ASN | A | 69 | 47.923 | 41.068 | 94.851 | 1.00 | 87.58 | C |
| ATOM | 442 | CB | ASN | A | 69 | 48.879 | 41.941 | 95.665 | 1.00 | 90.03 | C |
| ATOM | 443 | CG | ASN | A | 69 | 50.325 | 41.678 | 95.341 | 1.00 | 92.35 | C |
| ATOM | 444 | OD1 | ASN | A | 69 | 50.742 | 40.532 | 95.230 | 1.00 | 92.97 | O |
| ATOM | 445 | ND2 | ASN | A | 69 | 51.108 | 42.741 | 95.207 | 1.00 | 96.10 | N |
| ATOM | 446 | C | ASN | A | 69 | 46.583 | 41.047 | 95.573 | 1.00 | 88.00 | C |
| ATOM | 447 | O | ASN | A | 69 | 46.470 | 40.444 | 96.637 | 1.00 | 88.72 | O |
| ATOM | 448 | N | LEU | A | 70 | 45.578 | 41.712 | 95.018 | 1.00 | 88.56 | N |
| ATOM | 449 | CA | LEU | A | 70 | 44.279 | 41.735 | 95.662 | 1.00 | 90.75 | C |
| ATOM | 450 | CB | LEU | A | 70 | 43.728 | 43.167 | 95.664 | 1.00 | 94.36 | C |
| ATOM | 451 | CG | LEU | A | 70 | 44.595 | 44.165 | 96.460 | 1.00 | 96.96 | C |
| ATOM | 452 | CD1 | LEU | A | 70 | 43.965 | 45.556 | 96.427 | 1.00 | 98.03 | C |
| ATOM | 453 | CD2 | LEU | A | 70 | 44.752 | 43.686 | 97.905 | 1.00 | 96.55 | C |
| ATOM | 454 | C | LEU | A | 70 | 43.318 | 40.747 | 94.998 | 1.00 | 90.59 | C |
| ATOM | 455 | O | LEU | A | 70 | 42.105 | 40.778 | 95.229 | 1.00 | 90.68 | O |
| ATOM | 456 | N | LEU | A | 71 | 43.886 | 39.861 | 94.181 | 1.00 | 88.52 | N |
| ATOM | 457 | CA | LEU | A | 71 | 43.128 | 38.820 | 93.486 | 1.00 | 85.71 | C |
| ATOM | 458 | CB | LEU | A | 71 | 43.947 | 38.261 | 92.323 | 1.00 | 82.62 | C |
| ATOM | 459 | CG | LEU | A | 71 | 43.916 | 38.966 | 90.975 | 1.00 | 81.29 | C |
| ATOM | 460 | CD1 | LEU | A | 71 | 45.141 | 38.565 | 90.164 | 1.00 | 77.55 | C |
| ATOM | 461 | CD2 | LEU | A | 71 | 42.624 | 38.610 | 90.250 | 1.00 | 77.18 | C |
| ATOM | 462 | C | LEU | A | 71 | 42.835 | 37.685 | 94.465 | 1.00 | 84.84 | C |
| ATOM | 463 | O | LEU | A | 71 | 43.622 | 37.436 | 95.381 | 1.00 | 82.76 | O |
| ATOM | 464 | N | PRO | A | 72 | 41.698 | 36.985 | 94.292 | 1.00 | 84.38 | N |
| ATOM | 465 | CD | PRO | A | 72 | 40.666 | 37.118 | 93.247 | 1.00 | 83.45 | C |
| ATOM | 466 | CA | PRO | A | 72 | 41.380 | 35.880 | 95.205 | 1.00 | 85.05 | C |
| ATOM | 467 | CB | PRO | A | 72 | 40.218 | 35.179 | 94.500 | 1.00 | 82.70 | C |
| ATOM | 468 | CG | PRO | A | 72 | 39.518 | 36.311 | 93.814 | 1.00 | 82.63 | C |
| ATOM | 469 | C | PRO | A | 72 | 42.622 | 34.991 | 95.318 | 1.00 | 85.74 | C |
| ATOM | 470 | O | PRO | A | 72 | 43.354 | 34.833 | 94.338 | 1.00 | 86.42 | O |
| ATOM | 471 | N | ASP | A | 73 | 42.874 | 34.417 | 96.491 | 1.00 | 85.49 | N |
| ATOM | 472 | CA | ASP | A | 73 | 44.059 | 33.582 | 96.642 | 1.00 | 85.45 | C |
| ATOM | 473 | CB | ASP | A | 73 | 44.268 | 33.167 | 98.103 | 1.00 | 89.05 | C |
| ATOM | 474 | CG | ASP | A | 73 | 45.622 | 32.513 | 98.331 | 1.00 | 92.80 | C |
| ATOM | 475 | OD1 | ASP | A | 73 | 46.643 | 33.161 | 98.033 | 1.00 | 93.16 | O |
| ATOM | 476 | OD2 | ASP | A | 73 | 45.665 | 31.356 | 98.802 | 1.00 | 92.29 | O |
| ATOM | 477 | C | ASP | A | 73 | 44.000 | 32.345 | 95.754 | 1.00 | 82.63 | C |
| ATOM | 478 | O | ASP | A | 73 | 45.038 | 31.777 | 95.401 | 1.00 | 79.34 | O |
| ATOM | 479 | N | ARG | A | 74 | 42.794 | 31.929 | 95.379 | 1.00 | 80.62 | N |
| ATOM | 480 | CA | ARG | A | 74 | 42.664 | 30.758 | 94.522 | 1.00 | 80.42 | C |
| ATOM | 481 | CB | ARG | A | 74 | 41.201 | 30.286 | 94.448 | 1.00 | 81.78 | C |
| ATOM | 482 | CG | ARG | A | 74 | 40.217 | 31.254 | 93.816 | 1.00 | 85.31 | C |
| ATOM | 483 | CD | ARG | A | 74 | 38.883 | 30.540 | 93.609 | 1.00 | 87.56 | C |
| ATOM | 484 | NE | ARG | A | 74 | 37.857 | 31.371 | 92.985 | 1.00 | 90.18 | N |
| ATOM | 485 | CZ | ARG | A | 74 | 37.241 | 32.385 | 93.582 | 1.00 | 90.50 | C |
| ATOM | 486 | NH1 | ARG | A | 74 | 37.543 | 32.707 | 94.830 | 1.00 | 91.18 | N |
| ATOM | 487 | NH2 | ARG | A | 74 | 36.317 | 33.076 | 92.928 | 1.00 | 92.03 | N |
| ATOM | 488 | C | ARG | A | 74 | 43.212 | 31.045 | 93.123 | 1.00 | 78.15 | C |
| ATOM | 489 | O | ARG | A | 74 | 43.538 | 30.126 | 92.373 | 1.00 | 76.44 | O |
| ATOM | 490 | N | VAL | A | 75 | 43.306 | 32.324 | 92.772 | 1.00 | 75.45 | N |
| ATOM | 491 | CA | VAL | A | 75 | 43.844 | 32.718 | 91.476 | 1.00 | 71.04 | C |
| ATOM | 492 | CB | VAL | A | 75 | 43.122 | 33.953 | 90.910 | 1.00 | 69.91 | C |
| ATOM | 493 | CG1 | VAL | A | 75 | 43.744 | 34.355 | 89.581 | 1.00 | 64.92 | C |
| ATOM | 494 | CG2 | VAL | A | 75 | 41.641 | 33.652 | 90.735 | 1.00 | 66.58 | C |
| ATOM | 495 | C | VAL | A | 75 | 45.291 | 33.081 | 91.734 | 1.00 | 70.45 | C |
| ATOM | 496 | O | VAL | A | 75 | 46.188 | 32.705 | 90.991 | 1.00 | 68.83 | O |
| ATOM | 497 | N | LEU | A | 76 | 45.484 | 33.825 | 92.814 | 1.00 | 71.81 | N |
| ATOM | 498 | CA | LEU | A | 76 | 46.787 | 34.286 | 93.255 | 1.00 | 74.65 | C |
| ATOM | 499 | CB | LEU | A | 76 | 46.663 | 34.888 | 94.653 | 1.00 | 75.65 | C |
| ATOM | 500 | CG | LEU | A | 76 | 46.970 | 36.363 | 94.860 | 1.00 | 77.97 | C |
| ATOM | 501 | CD1 | LEU | A | 76 | 46.604 | 36.727 | 96.296 | 1.00 | 76.12 | C |
| ATOM | 502 | CD2 | LEU | A | 76 | 48.454 | 36.641 | 94.561 | 1.00 | 73.88 | C |
| ATOM | 503 | C | LEU | A | 76 | 47.809 | 33.151 | 93.305 | 1.00 | 74.80 | C |
| ATOM | 504 | O | LEU | A | 76 | 48.876 | 33.227 | 92.691 | 1.00 | 72.45 | O |
| ATOM | 505 | N | SER | A | 77 | 47.461 | 32.093 | 94.032 | 1.00 | 75.97 | N |
| ATOM | 506 | CA | SER | A | 77 | 48.337 | 30.940 | 94.223 | 1.00 | 77.39 | C |
| ATOM | 507 | CB | SER | A | 77 | 47.746 | 30.032 | 95.302 | 1.00 | 77.82 | C |
| ATOM | 508 | OG | SER | A | 77 | 46.452 | 29.592 | 94.930 | 1.00 | 82.02 | O |
| ATOM | 509 | C | SER | A | 77 | 48.703 | 30.086 | 93.005 | 1.00 | 77.66 | C |
| ATOM | 510 | O | SER | A | 77 | 49.630 | 29.264 | 93.093 | 1.00 | 80.01 | O |
| ATOM | 511 | N | THR | A | 78 | 48.002 | 30.257 | 91.882 | 1.00 | 75.12 | N |
| ATOM | 512 | CA | THR | A | 78 | 48.310 | 29.452 | 90.700 | 1.00 | 72.38 | C |
| ATOM | 513 | CB | THR | A | 78 | 47.358 | 29.760 | 89.520 | 1.00 | 72.55 | C |
| ATOM | 514 | OG1 | THR | A | 78 | 47.622 | 31.073 | 89.013 | 1.00 | 71.84 | O |
| ATOM | 515 | CG2 | THR | A | 78 | 45.920 | 29.675 | 89.968 | 1.00 | 70.77 | C |
| ATOM | 516 | C | THR | A | 78 | 49.742 | 29.676 | 90.224 | 1.00 | 70.61 | C |
| ATOM | 517 | O | THR | A | 78 | 50.276 | 30.775 | 90.340 | 1.00 | 72.01 | O |
| ATOM | 518 | N | PRO | A | 79 | 50.392 | 28.624 | 89.702 | 1.00 | 68.47 | N |
| ATOM | 519 | CD | PRO | A | 79 | 49.958 | 27.217 | 89.663 | 1.00 | 64.46 | C |
| ATOM | 520 | CA | PRO | A | 79 | 51.764 | 28.754 | 89.216 | 1.00 | 66.56 | C |
| ATOM | 521 | CB | PRO | A | 79 | 52.045 | 27.377 | 88.607 | 1.00 | 64.97 | C |
| ATOM | 522 | CG | PRO | A | 79 | 51.259 | 26.472 | 89.480 | 1.00 | 64.56 | C |
| ATOM | 523 | C | PRO | A | 79 | 51.888 | 29.876 | 88.186 | 1.00 | 65.26 | C |
| ATOM | 524 | O | PRO | A | 79 | 52.905 | 30.584 | 88.158 | 1.00 | 63.38 | O |
| ATOM | 525 | N | SER | A | 80 | 50.853 | 30.026 | 87.351 | 1.00 | 65.41 | N |
| ATOM | 526 | CA | SER | A | 80 | 50.813 | 31.038 | 86.290 | 1.00 | 64.44 | C |
| ATOM | 527 | CB | SER | A | 80 | 49.461 | 31.034 | 85.574 | 1.00 | 65.62 | C |
| ATOM | 528 | OG | SER | A | 80 | 48.896 | 29.753 | 85.466 | 1.00 | 74.77 | O |
| ATOM | 529 | C | SER | A | 80 | 50.994 | 32.417 | 86.874 | 1.00 | 62.98 | C |
| ATOM | 530 | O | SER | A | 80 | 52.004 | 33.076 | 86.673 | 1.00 | 58.07 | O |
| ATOM | 531 | N | VAL | A | 81 | 49.972 | 32.843 | 87.600 | 1.00 | 63.63 | N |
| ATOM | 532 | CA | VAL | A | 81 | 49.962 | 34.152 | 88.216 | 1.00 | 66.18 | C |
| ATOM | 533 | CB | VAL | A | 81 | 48.679 | 34.354 | 89.041 | 1.00 | 65.32 | C |
| ATOM | 534 | CG1 | VAL | A | 81 | 48.707 | 35.716 | 89.719 | 1.00 | 65.53 | C |
| ATOM | 535 | CG2 | VAL | A | 81 | 47.456 | 34.230 | 88.138 | 1.00 | 62.55 | C |
| ATOM | 536 | C | VAL | A | 81 | 51.178 | 34.388 | 89.094 | 1.00 | 67.24 | C |
| ATOM | 537 | O | VAL | A | 81 | 51.768 | 35.463 | 89.063 | 1.00 | 67.77 | O |
| ATOM | 538 | N | GLN | A | 82 | 51.560 | 33.377 | 89.862 | 1.00 | 67.29 | N |
| ATOM | 539 | CA | GLN | A | 82 | 52.706 | 33.503 | 90.748 | 1.00 | 68.46 | C |
| ATOM | 540 | CB | GLN | A | 82 | 52.834 | 32.247 | 91.618 | 1.00 | 73.80 | C |
| ATOM | 541 | CG | GLN | A | 82 | 53.164 | 32.515 | 93.087 | 1.00 | 81.81 | C |
| ATOM | 542 | CD | GLN | A | 82 | 52.176 | 33.467 | 93.754 | 1.00 | 88.54 | C |
| ATOM | 543 | OE1 | GLN | A | 82 | 50.973 | 33.391 | 93.518 | 1.00 | 92.32 | O |
| ATOM | 544 | NE2 | GLN | A | 82 | 52.684 | 34.360 | 94.602 | 1.00 | 92.11 | N |
| ATOM | 545 | C | GLN | A | 82 | 54.000 | 33.736 | 89.965 | 1.00 | 65.25 | C |
| ATOM | 546 | O | GLN | A | 82 | 54.821 | 34.570 | 90.343 | 1.00 | 66.00 | O |
| ATOM | 547 | N | LEU | A | 83 | 54.179 | 33.003 | 88.876 | 1.00 | 61.51 | N |
| ATOM | 548 | CA | LEU | A | 83 | 55.373 | 33.144 | 88.060 | 1.00 | 60.56 | C |
| ATOM | 549 | CB | LEU | A | 83 | 55.383 | 32.069 | 86.957 | 1.00 | 57.96 | C |
| ATOM | 550 | CG | LEU | A | 83 | 56.429 | 32.244 | 85.840 | 1.00 | 61.26 | C |
| ATOM | 551 | CD1 | LEU | A | 83 | 57.818 | 32.297 | 86.431 | 1.00 | 58.90 | C |
| ATOM | 552 | CD2 | LEU | A | 83 | 56.305 | 31.099 | 84.851 | 1.00 | 61.77 | C |
| ATOM | 553 | C | LEU | A | 83 | 55.453 | 34.542 | 87.438 | 1.00 | 61.39 | C |
| ATOM | 554 | O | LEU | A | 83 | 56.479 | 35.208 | 87.520 | 1.00 | 57.80 | O |
| ATOM | 555 | N | VAL | A | 84 | 54.365 | 34.965 | 86.801 | 1.00 | 63.07 | N |
| ATOM | 556 | CA | VAL | A | 84 | 54.294 | 36.273 | 86.159 | 1.00 | 63.72 | C |
| ATOM | 557 | CB | VAL | A | 84 | 52.922 | 36.466 | 85.437 | 1.00 | 62.56 | C |
| ATOM | 558 | CG1 | VAL | A | 84 | 52.667 | 37.948 | 85.177 | 1.00 | 61.80 | C |
| ATOM | 559 | CG2 | VAL | A | 84 | 52.908 | 35.707 | 84.123 | 1.00 | 58.05 | C |
| ATOM | 560 | C | VAL | A | 84 | 54.494 | 37.369 | 87.209 | 1.00 | 64.16 | C |
| ATOM | 561 | O | VAL | A | 84 | 55.198 | 38.362 | 86.972 | 1.00 | 61.34 | O |
| ATOM | 562 | N | GLN | A | 85 | 53.874 | 37.182 | 88.368 | 1.00 | 64.00 | N |
| ATOM | 563 | CA | GLN | A | 85 | 53.998 | 38.131 | 89.459 | 1.00 | 67.74 | C |
| ATOM | 564 | CB | GLN | A | 85 | 53.142 | 37.654 | 90.623 | 1.00 | 70.50 | C |
| ATOM | 565 | CG | GLN | A | 85 | 53.220 | 38.498 | 91.869 | 1.00 | 81.07 | C |
| ATOM | 566 | CD | GLN | A | 85 | 52.063 | 38.214 | 92.826 | 1.00 | 88.50 | C |
| ATOM | 567 | OE1 | GLN | A | 85 | 51.859 | 37.076 | 93.255 | 1.00 | 93.59 | O |
| ATOM | 568 | NE2 | GLN | A | 85 | 51.303 | 39.252 | 93.164 | 1.00 | 91.93 | N |
| ATOM | 569 | C | GLN | A | 85 | 55.454 | 38.312 | 89.899 | 1.00 | 68.46 | C |
| ATOM | 570 | O | GLN | A | 85 | 55.824 | 39.360 | 90.409 | 1.00 | 69.38 | O |
| ATOM | 571 | N | SER | A | 86 | 56.277 | 37.290 | 89.683 | 1.00 | 68.64 | N |
| ATOM | 572 | CA | SER | A | 86 | 57.686 | 37.346 | 90.058 | 1.00 | 68.56 | C |
| ATOM | 573 | CB | SER | A | 86 | 58.278 | 35.933 | 90.172 | 1.00 | 66.41 | C |
| ATOM | 574 | OG | SER | A | 86 | 58.555 | 35.371 | 88.900 | 1.00 | 61.74 | O |
| ATOM | 575 | C | SER | A | 86 | 58.497 | 38.159 | 89.047 | 1.00 | 70.54 | C |
| ATOM | 576 | O | SER | A | 86 | 59.490 | 38.800 | 89.410 | 1.00 | 70.94 | O |
| ATOM | 577 | N | TRP | A | 87 | 58.086 | 38.124 | 87.782 | 1.00 | 72.39 | N |
| ATOM | 578 | CA | TRP | A | 87 | 58.780 | 38.888 | 86.760 | 1.00 | 73.65 | C |
| ATOM | 579 | CB | TRP | A | 87 | 58.230 | 38.583 | 85.368 | 1.00 | 78.06 | C |
| ATOM | 580 | CG | TRP | A | 87 | 58.404 | 37.176 | 84.876 | 1.00 | 83.77 | C |
| ATOM | 581 | CD2 | TRP | A | 87 | 57.627 | 36.533 | 83.853 | 1.00 | 88.31 | C |
| ATOM | 582 | CE2 | TRP | A | 87 | 58.199 | 35.252 | 83.648 | 1.00 | 88.15 | C |
| ATOM | 583 | CE3 | TRP | A | 87 | 56.536 | 36.932 | 83.062 | 1.00 | 89.86 | C |
| ATOM | 584 | CD1 | TRP | A | 87 | 59.359 | 36.273 | 85.257 | 1.00 | 85.02 | C |
| ATOM | 585 | NE1 | TRP | A | 87 | 59.235 | 35.116 | 84.524 | 1.00 | 85.95 | N |
| ATOM | 586 | CZ2 | TRP | A | 87 | 57.670 | 34.344 | 82.709 | 1.00 | 88.37 | C |
| ATOM | 587 | CZ3 | TRP | A | 87 | 56.017 | 36.028 | 82.126 | 1.00 | 88.58 | C |
| ATOM | 588 | CH2 | TRP | A | 87 | 56.599 | 34.758 | 81.947 | 1.00 | 88.55 | C |
| ATOM | 589 | C | TRP | A | 87 | 58.586 | 40.380 | 87.044 | 1.00 | 70.82 | C |
| ATOM | 590 | O | TRP | A | 87 | 59.533 | 41.156 | 86.995 | 1.00 | 67.98 | O |
| ATOM | 591 | N | TYR | A | 88 | 57.357 | 40.781 | 87.346 | 1.00 | 69.80 | N |
| ATOM | 592 | CA | TYR | A | 88 | 57.082 | 42.185 | 87.617 | 1.00 | 72.37 | C |
| ATOM | 593 | CB | TYR | A | 88 | 55.576 | 42.422 | 87.755 | 1.00 | 73.10 | C |
| ATOM | 594 | CG | TYR | A | 88 | 54.850 | 42.473 | 86.429 | 1.00 | 75.81 | C |
| ATOM | 595 | CD1 | TYR | A | 88 | 54.607 | 41.311 | 85.699 | 1.00 | 75.90 | C |
| ATOM | 596 | CE1 | TYR | A | 88 | 54.004 | 41.365 | 84.442 | 1.00 | 77.92 | C |
| ATOM | 597 | CD2 | TYR | A | 88 | 54.465 | 43.695 | 85.868 | 1.00 | 76.71 | C |
| ATOM | 598 | CE2 | TYR | A | 88 | 53.865 | 43.760 | 84.613 | 1.00 | 75.90 | C |
| ATOM | 599 | CZ | TYR | A | 88 | 53.641 | 42.596 | 83.904 | 1.00 | 76.07 | C |
| ATOM | 600 | OH | TYR | A | 88 | 53.078 | 42.654 | 82.649 | 1.00 | 75.25 | O |
| ATOM | 601 | C | TYR | A | 88 | 57.807 | 42.683 | 88.862 | 1.00 | 73.40 | C |
| ATOM | 602 | O | TYR | A | 88 | 58.205 | 43.852 | 88.931 | 1.00 | 73.39 | O |
| ATOM | 603 | N | VAL | A | 89 | 57.984 | 41.797 | 89.842 | 1.00 | 72.12 | N |
| ATOM | 604 | CA | VAL | A | 89 | 58.680 | 42.162 | 91.075 | 1.00 | 69.44 | C |
| ATOM | 605 | CB | VAL | A | 89 | 58.604 | 41.045 | 92.137 | 1.00 | 69.63 | C |
| ATOM | 606 | CG1 | VAL | A | 89 | 59.593 | 41.332 | 93.264 | 1.00 | 68.70 | C |
| ATOM | 607 | CG2 | VAL | A | 89 | 57.192 | 40.938 | 92.701 | 1.00 | 67.27 | C |
| ATOM | 608 | C | VAL | A | 89 | 60.150 | 42.405 | 90.765 | 1.00 | 68.98 | C |
| ATOM | 609 | O | VAL | A | 89 | 60.760 | 43.344 | 91.268 | 1.00 | 66.22 | O |
| ATOM | 610 | N | GLN | A | 90 | 60.702 | 41.552 | 89.913 | 1.00 | 67.76 | N |
| ATOM | 611 | CA | GLN | A | 90 | 62.096 | 41.648 | 89.538 | 1.00 | 68.55 | C |
| ATOM | 612 | CB | GLN | A | 90 | 62.547 | 40.368 | 88.830 | 1.00 | 71.35 | C |
| ATOM | 613 | CG | GLN | A | 90 | 63.977 | 39.992 | 89.153 | 1.00 | 80.54 | C |
| ATOM | 614 | CD | GLN | A | 90 | 64.244 | 39.996 | 90.656 | 1.00 | 83.69 | C |
| ATOM | 615 | OE1 | GLN | A | 90 | 63.527 | 39.352 | 91.434 | 1.00 | 85.31 | O |
| ATOM | 616 | NE2 | GLN | A | 90 | 65.278 | 40.726 | 91.068 | 1.00 | 84.19 | N |
| ATOM | 617 | C | GLN | A | 90 | 62.344 | 42.838 | 88.639 | 1.00 | 67.59 | C |
| ATOM | 618 | O | GLN | A | 90 | 63.398 | 43.453 | 88.701 | 1.00 | 67.06 | O |
| ATOM | 619 | N | SER | A | 91 | 61.376 | 43.158 | 87.790 | 1.00 | 69.07 | N |
| ATOM | 620 | CA | SER | A | 91 | 61.515 | 44.292 | 86.879 | 1.00 | 67.15 | C |
| ATOM | 621 | CB | SER | A | 91 | 60.382 | 44.288 | 85.854 | 1.00 | 69.75 | C |
| ATOM | 622 | OG | SER | A | 91 | 60.562 | 43.249 | 84.907 | 1.00 | 71.23 | O |
| ATOM | 623 | C | SER | A | 91 | 61.508 | 45.603 | 87.657 | 1.00 | 64.20 | C |
| ATOM | 624 | O | SER | A | 91 | 62.231 | 46.531 | 87.326 | 1.00 | 58.21 | O |
| ATOM | 625 | N | LEU | A | 92 | 60.673 | 45.662 | 88.689 | 1.00 | 63.24 | N |
| ATOM | 626 | CA | LEU | A | 92 | 60.584 | 46.833 | 89.540 | 1.00 | 65.52 | C |
| ATOM | 627 | CB | LEU | A | 92 | 59.564 | 46.587 | 90.655 | 1.00 | 62.73 | C |
| ATOM | 628 | CG | LEU | A | 92 | 59.333 | 47.668 | 91.711 | 1.00 | 65.91 | C |
| ATOM | 629 | CD1 | LEU | A | 92 | 58.968 | 48.993 | 91.051 | 1.00 | 65.58 | C |
| ATOM | 630 | CD2 | LEU | A | 92 | 58.220 | 47.207 | 92.642 | 1.00 | 64.19 | C |
| ATOM | 631 | C | LEU | A | 92 | 61.969 | 47.080 | 90.132 | 1.00 | 68.94 | C |
| ATOM | 632 | O | LEU | A | 92 | 62.580 | 48.134 | 89.911 | 1.00 | 69.43 | O |
| ATOM | 633 | N | LEU | A | 93 | 62.470 | 46.086 | 90.862 | 1.00 | 69.79 | N |
| ATOM | 634 | CA | LEU | A | 93 | 63.788 | 46.167 | 91.492 | 1.00 | 72.72 | C |
| ATOM | 635 | CB | LEU | A | 93 | 64.119 | 44.843 | 92.193 | 1.00 | 70.03 | C |
| ATOM | 636 | CG | LEU | A | 93 | 63.043 | 44.322 | 93.153 | 1.00 | 70.68 | C |
| ATOM | 637 | CD1 | LEU | A | 93 | 63.436 | 42.964 | 93.700 | 1.00 | 64.03 | C |
| ATOM | 638 | CD2 | LEU | A | 93 | 62.826 | 45.325 | 94.266 | 1.00 | 67.96 | C |
| ATOM | 639 | C | LEU | A | 93 | 64.844 | 46.467 | 90.435 | 1.00 | 75.26 | C |
| ATOM | 640 | O | LEU | A | 93 | 65.837 | 47.138 | 90.713 | 1.00 | 74.25 | O |
| ATOM | 641 | N | ASP | A | 94 | 64.617 | 45.963 | 89.225 | 1.00 | 77.64 | N |
| ATOM | 642 | CA | ASP | A | 94 | 65.531 | 46.174 | 88.116 | 1.00 | 80.94 | C |
| ATOM | 643 | CB | ASP | A | 94 | 65.020 | 45.444 | 86.877 | 1.00 | 85.04 | C |
| ATOM | 644 | CG | ASP | A | 94 | 65.670 | 44.096 | 86.682 | 1.00 | 86.55 | C |
| ATOM | 645 | OD1 | ASP | A | 94 | 65.693 | 43.304 | 87.647 | 1.00 | 89.83 | O |
| ATOM | 646 | OD2 | ASP | A | 94 | 66.147 | 43.834 | 85.552 | 1.00 | 85.77 | O |
| ATOM | 647 | C | ASP | A | 94 | 65.709 | 47.651 | 87.783 | 1.00 | 82.52 | C |
| ATOM | 648 | O | ASP | A | 94 | 66.831 | 48.115 | 87.596 | 1.00 | 83.01 | O |
| ATOM | 649 | N | ILE | A | 95 | 64.604 | 48.388 | 87.713 | 1.00 | 83.11 | N |
| ATOM | 650 | CA | ILE | A | 95 | 64.657 | 49.805 | 87.366 | 1.00 | 85.22 | C |
| ATOM | 651 | CB | ILE | A | 95 | 63.303 | 50.279 | 86.752 | 1.00 | 85.58 | C |
| ATOM | 652 | CG2 | ILE | A | 95 | 63.388 | 51.736 | 86.342 | 1.00 | 83.75 | C |
| ATOM | 653 | CG1 | ILE | A | 95 | 62.973 | 49.452 | 85.506 | 1.00 | 85.89 | C |
| ATOM | 654 | CD1 | ILE | A | 95 | 61.676 | 49.821 | 84.837 | 1.00 | 88.17 | C |
| ATOM | 655 | C | ILE | A | 95 | 65.026 | 50.687 | 88.557 | 1.00 | 85.07 | C |
| ATOM | 656 | O | ILE | A | 95 | 65.590 | 51.762 | 88.384 | 1.00 | 83.34 | O |
| ATOM | 657 | N | MET | A | 96 | 64.713 | 50.224 | 89.762 | 1.00 | 86.89 | N |
| ATOM | 658 | CA | MET | A | 96 | 65.010 | 50.959 | 90.993 | 1.00 | 90.27 | C |
| ATOM | 659 | CB | MET | A | 96 | 64.679 | 50.083 | 92.205 | 1.00 | 90.33 | C |
| ATOM | 660 | CG | MET | A | 96 | 63.221 | 49.719 | 92.317 | 1.00 | 91.12 | C |
| ATOM | 661 | SD | MET | A | 96 | 62.232 | 51.159 | 92.709 | 1.00 | 93.56 | S |
| ATOM | 662 | CE | MET | A | 96 | 61.078 | 50.451 | 93.762 | 1.00 | 92.04 | C |
| ATOM | 663 | C | MET | A | 96 | 66.473 | 51.398 | 91.081 | 1.00 | 92.12 | C |
| ATOM | 664 | O | MET | A | 96 | 66.779 | 52.522 | 91.482 | 1.00 | 91.68 | O |
| ATOM | 665 | N | GLU | A | 97 | 67.365 | 50.491 | 90.701 | 1.00 | 93.47 | N |
| ATOM | 666 | CA | GLU | A | 97 | 68.808 | 50.714 | 90.723 | 1.00 | 94.32 | C |
| ATOM | 667 | CB | GLU | A | 97 | 69.497 | 49.666 | 89.840 | 1.00 | 96.77 | C |
| ATOM | 668 | CG | GLU | A | 97 | 69.095 | 48.230 | 90.136 | 1.00 | 99.69 | C |
| ATOM | 669 | CD | GLU | A | 97 | 69.694 | 47.704 | 91.430 | 1.00 | 102.13 | C |
| ATOM | 670 | OE1 | GLU | A | 97 | 70.916 | 47.433 | 91.462 | 1.00 | 103.91 | O |
| ATOM | 671 | OE2 | GLU | A | 97 | 68.943 | 47.564 | 92.421 | 1.00 | 103.20 | O |
| ATOM | 672 | C | GLU | A | 97 | 69.254 | 52.098 | 90.269 | 1.00 | 94.01 | C |
| ATOM | 673 | O | GLU | A | 97 | 70.319 | 52.568 | 90.667 | 1.00 | 93.60 | O |
| ATOM | 674 | N | PHE | A | 98 | 68.442 | 52.742 | 89.435 | 1.00 | 94.65 | N |
| ATOM | 675 | CA | PHE | A | 98 | 68.770 | 54.059 | 88.882 | 1.00 | 94.05 | C |
| ATOM | 676 | CB | PHE | A | 98 | 68.377 | 54.101 | 87.403 | 1.00 | 87.54 | C |
| ATOM | 677 | CG | PHE | A | 98 | 68.922 | 52.959 | 86.602 | 1.00 | 81.63 | C |
| ATOM | 678 | CD1 | PHE | A | 98 | 69.954 | 53.158 | 85.689 | 1.00 | 79.97 | C |
| ATOM | 679 | CD2 | PHE | A | 98 | 68.409 | 51.679 | 86.762 | 1.00 | 77.80 | C |
| ATOM | 680 | CE1 | PHE | A | 98 | 70.469 | 52.093 | 84.944 | 1.00 | 76.60 | C |
| ATOM | 681 | CE2 | PHE | A | 98 | 68.914 | 50.611 | 86.025 | 1.00 | 76.76 | C |
| ATOM | 682 | CZ | PHE | A | 98 | 69.946 | 50.823 | 85.115 | 1.00 | 77.77 | C |
| ATOM | 683 | C | PHE | A | 98 | 68.126 | 55.251 | 89.590 | 1.00 | 97.17 | C |
| ATOM | 684 | O | PHE | A | 98 | 68.331 | 56.394 | 89.180 | 1.00 | 96.87 | O |
| ATOM | 685 | N | LEU | A | 99 | 67.350 | 54.995 | 90.642 | 1.00 | 101.08 | N |
| ATOM | 686 | CA | LEU | A | 99 | 66.683 | 56.074 | 91.369 | 1.00 | 105.17 | C |
| ATOM | 687 | CB | LEU | A | 99 | 65.769 | 55.505 | 92.461 | 1.00 | 105.38 | C |
| ATOM | 688 | CG | LEU | A | 99 | 64.374 | 55.050 | 92.032 | 1.00 | 107.55 | C |
| ATOM | 689 | CD1 | LEU | A | 99 | 63.617 | 54.497 | 93.234 | 1.00 | 107.69 | C |
| ATOM | 690 | CD2 | LEU | A | 99 | 63.618 | 56.228 | 91.430 | 1.00 | 106.44 | C |
| ATOM | 691 | C | LEU | A | 99 | 67.625 | 57.104 | 91.985 | 1.00 | 107.97 | C |
| ATOM | 692 | O | LEU | A | 99 | 67.284 | 58.284 | 92.077 | 1.00 | 108.20 | O |
| ATOM | 693 | N | ASP | A | 100 | 68.807 | 56.664 | 92.403 | 1.00 | 110.22 | N |
| ATOM | 694 | CA | ASP | A | 100 | 69.771 | 57.569 | 93.018 | 1.00 | 111.44 | C |
| ATOM | 695 | CB | ASP | A | 100 | 70.337 | 56.948 | 94.302 | 1.00 | 113.19 | C |
| ATOM | 696 | CG | ASP | A | 100 | 71.018 | 55.606 | 94.060 | 1.00 | 115.48 | C |
| ATOM | 697 | OD1 | ASP | A | 100 | 70.316 | 54.624 | 93.730 | 1.00 | 116.09 | O |
| ATOM | 698 | OD2 | ASP | A | 100 | 72.258 | 55.534 | 94.195 | 1.00 | 115.33 | O |
| ATOM | 699 | C | ASP | A | 100 | 70.926 | 57.982 | 92.108 | 1.00 | 111.39 | C |
| ATOM | 700 | O | ASP | A | 100 | 71.705 | 58.862 | 92.464 | 1.00 | 111.58 | O |
| ATOM | 701 | N | LYS | A | 101 | 71.040 | 57.362 | 90.939 | 1.00 | 111.31 | N |
| ATOM | 702 | CA | LYS | A | 101 | 72.131 | 57.695 | 90.027 | 1.00 | 112.08 | C |
| ATOM | 703 | CB | LYS | A | 101 | 72.205 | 56.677 | 88.884 | 1.00 | 111.34 | C |
| ATOM | 704 | CG | LYS | A | 101 | 72.248 | 55.230 | 89.335 | 1.00 | 112.16 | C |
| ATOM | 705 | CD | LYS | A | 101 | 73.205 | 55.027 | 90.497 | 1.00 | 114.12 | C |
| ATOM | 706 | CE | LYS | A | 101 | 73.216 | 53.583 | 90.967 | 1.00 | 114.56 | C |
| ATOM | 707 | NZ | LYS | A | 101 | 73.869 | 53.439 | 92.292 | 1.00 | 115.35 | N |
| ATOM | 708 | C | LYS | A | 101 | 72.027 | 59.100 | 89.442 | 1.00 | 113.42 | C |
| ATOM | 709 | O | LYS | A | 101 | 71.020 | 59.786 | 89.611 | 1.00 | 114.01 | O |
| ATOM | 710 | N | ASP | A | 102 | 73.089 | 59.514 | 88.756 | 1.00 | 114.17 | N |
| ATOM | 711 | CA | ASP | A | 102 | 73.162 | 60.821 | 88.116 | 1.00 | 114.69 | C |
| ATOM | 712 | CB | ASP | A | 102 | 74.382 | 61.584 | 88.635 | 1.00 | 115.94 | C |
| ATOM | 713 | CG | ASP | A | 102 | 74.703 | 62.806 | 87.799 | 1.00 | 117.66 | C |
| ATOM | 714 | OD1 | ASP | A | 102 | 73.815 | 63.672 | 87.634 | 1.00 | 118.38 | O |
| ATOM | 715 | OD2 | ASP | A | 102 | 75.847 | 62.890 | 87.306 | 1.00 | 118.27 | O |
| ATOM | 716 | C | ASP | A | 102 | 73.252 | 60.611 | 86.607 | 1.00 | 114.54 | C |
| ATOM | 717 | O | ASP | A | 102 | 74.138 | 59.911 | 86.120 | 1.00 | 113.28 | O |
| ATOM | 718 | N | PRO | A | 103 | 72.337 | 61.236 | 85.848 | 1.00 | 115.04 | N |
| ATOM | 719 | CD | PRO | A | 103 | 71.206 | 62.036 | 86.352 | 1.00 | 114.01 | C |
| ATOM | 720 | CA | PRO | A | 103 | 72.276 | 61.126 | 84.389 | 1.00 | 116.36 | C |
| ATOM | 721 | CB | PRO | A | 103 | 70.864 | 61.592 | 84.077 | 1.00 | 115.74 | C |
| ATOM | 722 | CG | PRO | A | 103 | 70.659 | 62.663 | 85.083 | 1.00 | 114.44 | C |
| ATOM | 723 | C | PRO | A | 103 | 73.305 | 61.821 | 83.505 | 1.00 | 117.86 | C |
| ATOM | 724 | O | PRO | A | 103 | 73.234 | 61.690 | 82.283 | 1.00 | 119.16 | O |
| ATOM | 725 | N | GLU | A | 104 | 74.251 | 62.561 | 84.073 | 1.00 | 118.36 | N |
| ATOM | 726 | CA | GLU | A | 104 | 75.245 | 63.199 | 83.213 | 1.00 | 118.41 | C |
| ATOM | 727 | CB | GLU | A | 104 | 75.959 | 64.337 | 83.940 | 1.00 | 117.06 | C |
| ATOM | 728 | CG | GLU | A | 104 | 75.541 | 65.736 | 83.499 | 1.00 | 117.72 | C |
| ATOM | 729 | CD | GLU | A | 104 | 74.188 | 66.147 | 84.037 | 1.00 | 117.69 | C |
| ATOM | 730 | OE1 | GLU | A | 104 | 73.806 | 65.638 | 85.109 | 1.00 | 117.36 | O |
| ATOM | 731 | OE2 | GLU | A | 104 | 73.519 | 66.990 | 83.400 | 1.00 | 116.73 | O |
| ATOM | 732 | C | GLU | A | 104 | 76.258 | 62.153 | 82.760 | 1.00 | 119.40 | C |
| ATOM | 733 | O | GLU | A | 104 | 77.049 | 62.397 | 81.851 | 1.00 | 119.04 | O |
| ATOM | 734 | N | ASP | A | 105 | 76.219 | 60.988 | 83.403 | 1.00 | 120.73 | N |
| ATOM | 735 | CA | ASP | A | 105 | 77.121 | 59.886 | 83.075 | 1.00 | 122.57 | C |
| ATOM | 736 | CB | ASP | A | 105 | 77.232 | 58.931 | 84.277 | 1.00 | 123.70 | C |
| ATOM | 737 | CG | ASP | A | 105 | 78.323 | 57.874 | 84.104 | 1.00 | 124.74 | C |
| ATOM | 738 | OD1 | ASP | A | 105 | 79.510 | 58.241 | 83.979 | 1.00 | 124.64 | O |
| ATOM | 739 | OD2 | ASP | A | 105 | 77.997 | 56.667 | 84.100 | 1.00 | 126.66 | O |
| ATOM | 740 | C | ASP | A | 105 | 76.567 | 59.148 | 81.851 | 1.00 | 123.32 | C |
| ATOM | 741 | O | ASP | A | 105 | 75.587 | 58.410 | 81.948 | 1.00 | 123.18 | O |
| ATOM | 742 | N | HIS | A | 106 | 77.198 | 59.360 | 80.699 | 1.00 | 124.06 | N |
| ATOM | 743 | CA | HIS | A | 106 | 76.784 | 58.727 | 79.446 | 1.00 | 124.58 | C |
| ATOM | 744 | CB | HIS | A | 106 | 77.739 | 59.136 | 78.314 | 1.00 | 127.05 | C |
| ATOM | 745 | CG | HIS | A | 106 | 77.504 | 60.520 | 77.785 | 1.00 | 129.43 | C |
| ATOM | 746 | CD2 | HIS | A | 106 | 77.576 | 61.016 | 76.527 | 1.00 | 129.76 | C |
| ATOM | 747 | ND1 | HIS | A | 106 | 77.166 | 61.584 | 78.595 | 1.00 | 129.76 | N |
| ATOM | 748 | CE1 | HIS | A | 106 | 77.039 | 62.674 | 77.859 | 1.00 | 129.96 | C |
| ATOM | 749 | NE2 | HIS | A | 106 | 77.284 | 62.357 | 76.599 | 1.00 | 129.78 | N |
| ATOM | 750 | C | HIS | A | 106 | 76.744 | 57.207 | 79.556 | 1.00 | 123.80 | C |
| ATOM | 751 | O | HIS | A | 106 | 76.388 | 56.519 | 78.601 | 1.00 | 124.55 | O |
| ATOM | 752 | N | ARG | A | 107 | 77.106 | 56.693 | 80.730 | 1.00 | 122.28 | N |
| ATOM | 753 | CA | ARG | A | 107 | 77.130 | 55.257 | 80.987 | 1.00 | 120.51 | C |
| ATOM | 754 | CB | ARG | A | 107 | 78.490 | 54.874 | 81.589 | 1.00 | 121.17 | C |
| ATOM | 755 | CG | ARG | A | 107 | 78.871 | 53.399 | 81.482 | 1.00 | 123.17 | C |
| ATOM | 756 | CD | ARG | A | 107 | 80.393 | 53.250 | 81.531 | 1.00 | 125.78 | C |
| ATOM | 757 | NE | ARG | A | 107 | 80.850 | 51.861 | 81.571 | 1.00 | 128.63 | N |
| ATOM | 758 | CZ | ARG | A | 107 | 81.009 | 51.150 | 82.687 | 1.00 | 130.09 | C |
| ATOM | 759 | NH1 | ARG | A | 107 | 80.748 | 51.692 | 83.871 | 1.00 | 130.63 | N |
| ATOM | 760 | NH2 | ARG | A | 107 | 81.439 | 49.894 | 82.621 | 1.00 | 130.23 | N |
| ATOM | 761 | C | ARG | A | 107 | 75.983 | 54.868 | 81.933 | 1.00 | 118.69 | C |
| ATOM | 762 | O | ARG | A | 107 | 75.529 | 53.724 | 81.936 | 1.00 | 118.18 | O |
| ATOM | 763 | N | THR | A | 108 | 75.514 | 55.833 | 82.722 | 1.00 | 116.38 | N |
| ATOM | 764 | CA | THR | A | 108 | 74.413 | 55.608 | 83.657 | 1.00 | 114.64 | C |
| ATOM | 765 | CB | THR | A | 108 | 74.216 | 56.813 | 84.624 | 1.00 | 114.70 | C |
| ATOM | 766 | OG1 | THR | A | 108 | 75.268 | 56.831 | 85.596 | 1.00 | 116.71 | O |
| ATOM | 767 | CG2 | THR | A | 108 | 72.873 | 56.722 | 85.343 | 1.00 | 113.24 | C |
| ATOM | 768 | C | THR | A | 108 | 73.112 | 55.405 | 82.890 | 1.00 | 113.40 | C |
| ATOM | 769 | O | THR | A | 108 | 72.238 | 54.653 | 83.319 | 1.00 | 113.28 | O |
| ATOM | 770 | N | LEU | A | 109 | 72.986 | 56.081 | 81.754 | 1.00 | 110.81 | N |
| ATOM | 771 | CA | LEU | A | 109 | 71.779 | 55.970 | 80.956 | 1.00 | 108.36 | C |
| ATOM | 772 | CB | LEU | A | 109 | 71.417 | 57.335 | 80.357 | 1.00 | 107.01 | C |
| ATOM | 773 | CG | LEU | A | 109 | 72.498 | 58.205 | 79.718 | 1.00 | 104.11 | C |
| ATOM | 774 | CD1 | LEU | A | 109 | 73.085 | 57.513 | 78.497 | 1.00 | 104.18 | C |
| ATOM | 775 | CD2 | LEU | A | 109 | 71.884 | 59.534 | 79.330 | 1.00 | 102.37 | C |
| ATOM | 776 | C | LEU | A | 109 | 71.846 | 54.896 | 79.870 | 1.00 | 107.35 | C |
| ATOM | 777 | O | LEU | A | 109 | 70.815 | 54.478 | 79.342 | 1.00 | 108.31 | O |
| ATOM | 778 | N | SER | A | 110 | 73.050 | 54.439 | 79.541 | 1.00 | 104.17 | N |
| ATOM | 779 | CA | SER | A | 110 | 73.195 | 53.393 | 78.535 | 1.00 | 101.78 | C |
| ATOM | 780 | CB | SER | A | 110 | 74.590 | 53.419 | 77.918 | 1.00 | 100.08 | C |
| ATOM | 781 | OG | SER | A | 110 | 75.561 | 53.013 | 78.860 | 1.00 | 99.39 | O |
| ATOM | 782 | C | SER | A | 110 | 72.959 | 52.053 | 79.215 | 1.00 | 101.45 | C |
| ATOM | 783 | O | SER | A | 110 | 72.802 | 51.023 | 78.563 | 1.00 | 100.25 | O |
| ATOM | 784 | N | GLN | A | 111 | 72.949 | 52.078 | 80.542 | 1.00 | 102.05 | N |
| ATOM | 785 | CA | GLN | A | 111 | 72.707 | 50.880 | 81.332 | 1.00 | 102.91 | C |
| ATOM | 786 | CB | GLN | A | 111 | 73.473 | 50.943 | 82.647 | 1.00 | 105.30 | C |
| ATOM | 787 | CG | GLN | A | 111 | 74.960 | 50.763 | 82.483 | 1.00 | 108.65 | C |
| ATOM | 788 | CD | GLN | A | 111 | 75.731 | 51.183 | 83.717 | 1.00 | 111.55 | C |
| ATOM | 789 | OE1 | GLN | A | 111 | 76.933 | 50.945 | 83.819 | 1.00 | 113.16 | O |
| ATOM | 790 | NE2 | GLN | A | 111 | 75.043 | 51.821 | 84.662 | 1.00 | 111.41 | N |
| ATOM | 791 | C | GLN | A | 111 | 71.218 | 50.819 | 81.608 | 1.00 | 102.11 | C |
| ATOM | 792 | O | GLN | A | 111 | 70.679 | 49.765 | 81.954 | 1.00 | 104.47 | O |
| ATOM | 793 | N | PHE | A | 112 | 70.564 | 51.969 | 81.461 | 1.00 | 98.47 | N |
| ATOM | 794 | CA | PHE | A | 112 | 69.129 | 52.064 | 81.660 | 1.00 | 94.23 | C |
| ATOM | 795 | CB | PHE | A | 112 | 68.707 | 53.526 | 81.795 | 1.00 | 91.59 | C |
| ATOM | 796 | CG | PHE | A | 112 | 67.266 | 53.701 | 82.175 | 1.00 | 91.23 | C |
| ATOM | 797 | CD1 | PHE | A | 112 | 66.799 | 53.227 | 83.397 | 1.00 | 90.39 | C |
| ATOM | 798 | CD2 | PHE | A | 112 | 66.375 | 54.334 | 81.317 | 1.00 | 90.44 | C |
| ATOM | 799 | CE1 | PHE | A | 112 | 65.465 | 53.377 | 83.760 | 1.00 | 90.36 | C |
| ATOM | 800 | CE2 | PHE | A | 112 | 65.038 | 54.490 | 81.668 | 1.00 | 90.17 | C |
| ATOM | 801 | CZ | PHE | A | 112 | 64.582 | 54.013 | 82.889 | 1.00 | 91.02 | C |
| ATOM | 802 | C | PHE | A | 112 | 68.514 | 51.453 | 80.409 | 1.00 | 92.54 | C |
| ATOM | 803 | O | PHE | A | 112 | 67.571 | 50.672 | 80.477 | 1.00 | 91.05 | O |
| ATOM | 804 | N | THR | A | 113 | 69.086 | 51.802 | 79.263 | 1.00 | 90.15 | N |
| ATOM | 805 | CA | THR | A | 113 | 68.613 | 51.289 | 77.988 | 1.00 | 89.72 | C |
| ATOM | 806 | CB | THR | A | 113 | 69.419 | 51.880 | 76.815 | 1.00 | 87.02 | C |
| ATOM | 807 | OG1 | THR | A | 113 | 69.377 | 53.310 | 76.882 | 1.00 | 86.18 | O |
| ATOM | 808 | CG2 | THR | A | 113 | 68.840 | 51.421 | 75.488 | 1.00 | 85.06 | C |
| ATOM | 809 | C | THR | A | 113 | 68.723 | 49.770 | 77.955 | 1.00 | 90.50 | C |
| ATOM | 810 | O | THR | A | 113 | 67.798 | 49.080 | 77.536 | 1.00 | 91.70 | O |
| ATOM | 811 | N | ASP | A | 114 | 69.860 | 49.254 | 78.406 | 1.00 | 90.46 | N |
| ATOM | 812 | CA | ASP | A | 114 | 70.079 | 47.817 | 78.434 | 1.00 | 90.05 | C |
| ATOM | 813 | CB | ASP | A | 114 | 71.538 | 47.506 | 78.765 | 1.00 | 92.97 | C |
| ATOM | 814 | CG | ASP | A | 114 | 72.469 | 47.827 | 77.617 | 1.00 | 95.86 | C |
| ATOM | 815 | OD1 | ASP | A | 114 | 73.676 | 47.514 | 77.712 | 1.00 | 93.04 | O |
| ATOM | 816 | OD2 | ASP | A | 114 | 71.987 | 48.396 | 76.614 | 1.00 | 100.69 | O |
| ATOM | 817 | C | ASP | A | 114 | 69.175 | 47.167 | 79.460 | 1.00 | 88.13 | C |
| ATOM | 818 | O | ASP | A | 114 | 68.793 | 46.005 | 79.319 | 1.00 | 86.54 | O |
| ATOM | 819 | N | ALA | A | 115 | 68.849 | 47.928 | 80.498 | 1.00 | 85.70 | N |
| ATOM | 820 | CA | ALA | A | 115 | 67.987 | 47.449 | 81.561 | 1.00 | 85.24 | C |
| ATOM | 821 | CB | ALA | A | 115 | 68.051 | 48.404 | 82.744 | 1.00 | 84.62 | C |
| ATOM | 822 | C | ALA | A | 115 | 66.555 | 47.327 | 81.057 | 1.00 | 84.60 | C |
| ATOM | 823 | O | ALA | A | 115 | 65.809 | 46.453 | 81.494 | 1.00 | 86.02 | O |
| ATOM | 824 | N | LEU | A | 116 | 66.171 | 48.206 | 80.137 | 1.00 | 82.49 | N |
| ATOM | 825 | CA | LEU | A | 116 | 64.821 | 48.180 | 79.592 | 1.00 | 79.96 | C |
| ATOM | 826 | CB | LEU | A | 116 | 64.510 | 49.504 | 78.875 | 1.00 | 77.14 | C |
| ATOM | 827 | CG | LEU | A | 116 | 64.421 | 50.724 | 79.799 | 1.00 | 72.61 | C |
| ATOM | 828 | CD1 | LEU | A | 116 | 64.007 | 51.936 | 78.991 | 1.00 | 71.97 | C |
| ATOM | 829 | CD2 | LEU | A | 116 | 63.428 | 50.463 | 80.911 | 1.00 | 69.64 | C |
| ATOM | 830 | C | LEU | A | 116 | 64.658 | 46.993 | 78.649 | 1.00 | 79.27 | C |
| ATOM | 831 | O | LEU | A | 116 | 63.698 | 46.223 | 78.776 | 1.00 | 77.75 | O |
| ATOM | 832 | N | VAL | A | 117 | 65.602 | 46.838 | 77.722 | 1.00 | 79.00 | N |
| ATOM | 833 | CA | VAL | A | 117 | 65.556 | 45.737 | 76.764 | 1.00 | 79.66 | C |
| ATOM | 834 | CB | VAL | A | 117 | 66.789 | 45.752 | 75.845 | 1.00 | 78.04 | C |
| ATOM | 835 | CG1 | VAL | A | 117 | 66.719 | 44.606 | 74.856 | 1.00 | 76.53 | C |
| ATOM | 836 | CG2 | VAL | A | 117 | 66.859 | 47.080 | 75.103 | 1.00 | 78.02 | C |
| ATOM | 837 | C | VAL | A | 117 | 65.474 | 44.383 | 77.467 | 1.00 | 79.45 | C |
| ATOM | 838 | O | VAL | A | 117 | 64.830 | 43.452 | 76.981 | 1.00 | 78.56 | O |
| ATOM | 839 | N | THR | A | 118 | 66.120 | 44.286 | 78.623 | 1.00 | 80.53 | N |
| ATOM | 840 | CA | THR | A | 118 | 66.125 | 43.047 | 79.395 | 1.00 | 83.36 | C |
| ATOM | 841 | CB | THR | A | 118 | 67.263 | 43.052 | 80.448 | 1.00 | 82.62 | C |
| ATOM | 842 | OG1 | THR | A | 118 | 68.531 | 43.077 | 79.783 | 1.00 | 82.95 | O |
| ATOM | 843 | CG2 | THR | A | 118 | 67.186 | 41.823 | 81.328 | 1.00 | 79.42 | C |
| ATOM | 844 | C | THR | A | 118 | 64.784 | 42.817 | 80.099 | 1.00 | 84.80 | C |
| ATOM | 845 | O | THR | A | 118 | 64.357 | 41.683 | 80.294 | 1.00 | 86.44 | O |
| ATOM | 846 | N | ILE | A | 119 | 64.112 | 43.898 | 80.473 | 1.00 | 83.49 | N |
| ATOM | 847 | CA | ILE | A | 119 | 62.824 | 43.790 | 81.143 | 1.00 | 83.44 | C |
| ATOM | 848 | CB | ILE | A | 119 | 62.466 | 45.111 | 81.872 | 1.00 | 82.48 | C |
| ATOM | 849 | CG2 | ILE | A | 119 | 61.059 | 45.036 | 82.455 | 1.00 | 81.94 | C |
| ATOM | 850 | CG1 | ILE | A | 119 | 63.469 | 45.371 | 82.993 | 1.00 | 80.15 | C |
| ATOM | 851 | CD1 | ILE | A | 119 | 63.235 | 46.653 | 83.755 | 1.00 | 77.48 | C |
| ATOM | 852 | C | ILE | A | 119 | 61.732 | 43.457 | 80.125 | 1.00 | 85.09 | C |
| ATOM | 853 | O | ILE | A | 119 | 60.810 | 42.692 | 80.410 | 1.00 | 84.85 | O |
| ATOM | 854 | N | ARG | A | 120 | 61.859 | 44.018 | 78.928 | 1.00 | 86.81 | N |
| ATOM | 855 | CA | ARG | A | 120 | 60.887 | 43.796 | 77.865 | 1.00 | 89.62 | C |
| ATOM | 856 | CB | ARG | A | 120 | 61.248 | 44.645 | 76.649 | 1.00 | 91.47 | C |
| ATOM | 857 | CG | ARG | A | 120 | 60.268 | 44.537 | 75.494 | 1.00 | 95.24 | C |
| ATOM | 858 | CD | ARG | A | 120 | 60.714 | 45.440 | 74.357 | 1.00 | 99.62 | C |
| ATOM | 859 | NE | ARG | A | 120 | 62.016 | 45.042 | 73.828 | 1.00 | 101.96 | N |
| ATOM | 860 | CZ | ARG | A | 120 | 62.788 | 45.816 | 73.072 | 1.00 | 102.80 | C |
| ATOM | 861 | NH1 | ARG | A | 120 | 62.392 | 47.042 | 72.756 | 1.00 | 103.03 | N |
| ATOM | 862 | NH2 | ARG | A | 120 | 63.954 | 45.361 | 72.625 | 1.00 | 102.74 | N |
| ATOM | 863 | C | ARG | A | 120 | 60.803 | 42.332 | 77.450 | 1.00 | 89.33 | C |
| ATOM | 864 | O | ARG | A | 120 | 59.712 | 41.763 | 77.331 | 1.00 | 86.98 | O |
| ATOM | 865 | N | ASN | A | 121 | 61.967 | 41.734 | 77.224 | 1.00 | 88.76 | N |
| ATOM | 866 | CA | ASN | A | 121 | 62.037 | 40.345 | 76.815 | 1.00 | 88.62 | C |
| ATOM | 867 | CB | ASN | A | 121 | 63.435 | 40.041 | 76.266 | 1.00 | 89.28 | C |
| ATOM | 868 | CG | ASN | A | 121 | 63.846 | 40.991 | 75.152 | 1.00 | 90.85 | C |
| ATOM | 869 | OD1 | ASN | A | 121 | 63.005 | 41.605 | 74.503 | 1.00 | 90.45 | O |
| ATOM | 870 | ND2 | ASN | A | 121 | 65.146 | 41.098 | 74.916 | 1.00 | 91.76 | N |
| ATOM | 871 | C | ASN | A | 121 | 61.673 | 39.382 | 77.950 | 1.00 | 87.42 | C |
| ATOM | 872 | O | ASN | A | 121 | 61.252 | 38.252 | 77.704 | 1.00 | 87.11 | O |
| ATOM | 873 | N | ARG | A | 122 | 61.816 | 39.837 | 79.191 | 1.00 | 85.35 | N |
| ATOM | 874 | CA | ARG | A | 122 | 61.485 | 39.014 | 80.352 | 1.00 | 83.60 | C |
| ATOM | 875 | CB | ARG | A | 122 | 61.963 | 39.701 | 81.634 | 1.00 | 84.50 | C |
| ATOM | 876 | CG | ARG | A | 122 | 61.557 | 38.996 | 82.935 | 1.00 | 85.83 | C |
| ATOM | 877 | CD | ARG | A | 122 | 61.994 | 39.807 | 84.147 | 1.00 | 87.76 | C |
| ATOM | 878 | NE | ARG | A | 122 | 63.433 | 40.030 | 84.122 | 1.00 | 90.74 | N |
| ATOM | 879 | CZ | ARG | A | 122 | 64.041 | 41.104 | 84.612 | 1.00 | 91.44 | C |
| ATOM | 880 | NH1 | ARG | A | 122 | 63.333 | 42.072 | 85.178 | 1.00 | 91.48 | N |
| ATOM | 881 | NH2 | ARG | A | 122 | 65.360 | 41.213 | 84.518 | 1.00 | 91.56 | N |
| ATOM | 882 | C | ARG | A | 122 | 59.977 | 38.769 | 80.438 | 1.00 | 82.01 | C |
| ATOM | 883 | O | ARG | A | 122 | 59.537 | 37.760 | 81.001 | 1.00 | 80.58 | O |
| ATOM | 884 | N | HIS | A | 123 | 59.197 | 39.698 | 79.882 | 1.00 | 79.14 | N |
| ATOM | 885 | CA | HIS | A | 123 | 57.736 | 39.613 | 79.886 | 1.00 | 74.44 | C |
| ATOM | 886 | CB | HIS | A | 123 | 57.148 | 40.977 | 80.268 | 1.00 | 74.18 | C |
| ATOM | 887 | CG | HIS | A | 123 | 57.482 | 41.403 | 81.661 | 1.00 | 72.74 | C |
| ATOM | 888 | CD2 | HIS | A | 123 | 58.632 | 41.882 | 82.190 | 1.00 | 70.84 | C |
| ATOM | 889 | ND1 | HIS | A | 123 | 56.577 | 41.337 | 82.700 | 1.00 | 71.84 | N |
| ATOM | 890 | CE1 | HIS | A | 123 | 57.158 | 41.762 | 83.809 | 1.00 | 69.78 | C |
| ATOM | 891 | NE2 | HIS | A | 123 | 58.403 | 42.097 | 83.527 | 1.00 | 71.44 | N |
| ATOM | 892 | C | HIS | A | 123 | 57.180 | 39.191 | 78.533 | 1.00 | 72.06 | C |
| ATOM | 893 | O | HIS | A | 123 | 55.971 | 39.227 | 78.316 | 1.00 | 70.69 | O |
| ATOM | 894 | N | ASN | A | 124 | 58.056 | 38.781 | 77.625 | 1.00 | 69.88 | N |
| ATOM | 895 | CA | ASN | A | 124 | 57.601 | 38.422 | 76.297 | 1.00 | 70.67 | C |
| ATOM | 896 | CB | ASN | A | 124 | 58.798 | 38.111 | 75.397 | 1.00 | 70.90 | C |
| ATOM | 897 | CG | ASN | A | 124 | 58.796 | 38.952 | 74.139 | 1.00 | 76.37 | C |
| ATOM | 898 | OD1 | ASN | A | 124 | 59.485 | 39.974 | 74.047 | 1.00 | 78.02 | O |
| ATOM | 899 | ND2 | ASN | A | 124 | 57.993 | 38.541 | 73.164 | 1.00 | 80.20 | N |
| ATOM | 900 | C | ASN | A | 124 | 56.550 | 37.315 | 76.163 | 1.00 | 70.34 | C |
| ATOM | 901 | O | ASN | A | 124 | 55.841 | 37.273 | 75.157 | 1.00 | 70.50 | O |
| ATOM | 902 | N | ASP | A | 125 | 56.427 | 36.434 | 77.157 | 1.00 | 69.92 | N |
| ATOM | 903 | CA | ASP | A | 125 | 55.452 | 35.344 | 77.073 | 1.00 | 67.85 | C |
| ATOM | 904 | CB | ASP | A | 125 | 56.153 | 33.989 | 77.217 | 1.00 | 70.65 | C |
| ATOM | 905 | CG | ASP | A | 125 | 57.011 | 33.648 | 76.018 | 1.00 | 74.28 | C |
| ATOM | 906 | OD1 | ASP | A | 125 | 56.518 | 33.781 | 74.872 | 1.00 | 76.95 | O |
| ATOM | 907 | OD2 | ASP | A | 125 | 58.171 | 33.235 | 76.217 | 1.00 | 76.22 | O |
| ATOM | 908 | C | ASP | A | 125 | 54.313 | 35.426 | 78.079 | 1.00 | 65.82 | C |
| ATOM | 909 | O | ASP | A | 125 | 53.735 | 34.413 | 78.443 | 1.00 | 64.69 | O |
| ATOM | 910 | N | VAL | A | 126 | 53.971 | 36.638 | 78.498 | 1.00 | 61.66 | N |
| ATOM | 911 | CA | VAL | A | 126 | 52.912 | 36.845 | 79.472 | 1.00 | 58.03 | C |
| ATOM | 912 | CB | VAL | A | 126 | 52.803 | 38.337 | 79.858 | 1.00 | 58.61 | C |
| ATOM | 913 | CG1 | VAL | A | 126 | 51.634 | 38.549 | 80.805 | 1.00 | 56.47 | C |
| ATOM | 914 | CG2 | VAL | A | 126 | 54.079 | 38.797 | 80.518 | 1.00 | 59.43 | C |
| ATOM | 915 | C | VAL | A | 126 | 51.527 | 36.366 | 79.051 | 1.00 | 56.94 | C |
| ATOM | 916 | O | VAL | A | 126 | 50.821 | 35.738 | 79.829 | 1.00 | 55.78 | O |
| ATOM | 917 | N | VAL | A | 127 | 51.123 | 36.691 | 77.832 | 1.00 | 54.90 | N |
| ATOM | 918 | CA | VAL | A | 127 | 49.807 | 36.301 | 77.352 | 1.00 | 52.45 | C |
| ATOM | 919 | CB | VAL | A | 127 | 49.560 | 36.813 | 75.887 | 1.00 | 54.75 | C |
| ATOM | 920 | CG1 | VAL | A | 127 | 48.136 | 36.444 | 75.438 | 1.00 | 46.95 | C |
| ATOM | 921 | CG2 | VAL | A | 127 | 49.779 | 38.333 | 75.817 | 1.00 | 47.60 | C |
| ATOM | 922 | C | VAL | A | 127 | 49.595 | 34.791 | 77.444 | 1.00 | 51.60 | C |
| ATOM | 923 | O | VAL | A | 127 | 48.654 | 34.335 | 78.093 | 1.00 | 48.90 | O |
| ATOM | 924 | N | PRO | A | 128 | 50.468 | 33.994 | 76.805 | 1.00 | 51.04 | N |
| ATOM | 925 | CD | PRO | A | 128 | 51.629 | 34.399 | 75.999 | 1.00 | 49.80 | C |
| ATOM | 926 | CA | PRO | A | 128 | 50.342 | 32.530 | 76.843 | 1.00 | 51.17 | C |
| ATOM | 927 | CB | PRO | A | 128 | 51.605 | 32.059 | 76.144 | 1.00 | 51.69 | C |
| ATOM | 928 | CG | PRO | A | 128 | 51.888 | 33.150 | 75.187 | 1.00 | 54.34 | C |
| ATOM | 929 | C | PRO | A | 128 | 50.285 | 32.010 | 78.281 | 1.00 | 52.95 | C |
| ATOM | 930 | O | PRO | A | 128 | 49.462 | 31.147 | 78.619 | 1.00 | 52.02 | O |
| ATOM | 931 | N | THR | A | 129 | 51.180 | 32.548 | 79.112 | 1.00 | 50.07 | N |
| ATOM | 932 | CA | THR | A | 129 | 51.290 | 32.186 | 80.526 | 1.00 | 51.10 | C |
| ATOM | 933 | CB | THR | A | 129 | 52.454 | 32.923 | 81.219 | 1.00 | 53.43 | C |
| ATOM | 934 | OG1 | THR | A | 129 | 53.695 | 32.569 | 80.597 | 1.00 | 56.21 | O |
| ATOM | 935 | CG2 | THR | A | 129 | 52.500 | 32.549 | 82.689 | 1.00 | 51.44 | C |
| ATOM | 936 | C | THR | A | 129 | 50.040 | 32.486 | 81.318 | 1.00 | 52.02 | C |
| ATOM | 937 | O | THR | A | 129 | 49.586 | 31.665 | 82.104 | 1.00 | 51.36 | O |
| ATOM | 938 | N | MET | A | 130 | 49.493 | 33.677 | 81.119 | 1.00 | 51.03 | N |
| ATOM | 939 | CA | MET | A | 130 | 48.280 | 34.052 | 81.818 | 1.00 | 51.10 | C |
| ATOM | 940 | CB | MET | A | 130 | 47.983 | 35.525 | 81.588 | 1.00 | 51.66 | C |
| ATOM | 941 | CG | MET | A | 130 | 47.484 | 36.228 | 82.813 | 1.00 | 56.02 | C |
| ATOM | 942 | SD | MET | A | 130 | 48.790 | 36.426 | 84.021 | 1.00 | 61.04 | S |
| ATOM | 943 | CE | MET | A | 130 | 47.887 | 37.064 | 85.365 | 1.00 | 55.09 | C |
| ATOM | 944 | C | MET | A | 130 | 47.118 | 33.194 | 81.301 | 1.00 | 50.87 | C |
| ATOM | 945 | O | MET | A | 130 | 46.241 | 32.780 | 82.059 | 1.00 | 47.89 | O |
| ATOM | 946 | N | ALA | A | 131 | 47.133 | 32.925 | 79.999 | 1.00 | 51.34 | N |
| ATOM | 947 | CA | ALA | A | 131 | 46.111 | 32.109 | 79.357 | 1.00 | 53.80 | C |
| ATOM | 948 | CB | ALA | A | 131 | 46.431 | 31.943 | 77.871 | 1.00 | 49.80 | C |
| ATOM | 949 | C | ALA | A | 131 | 46.011 | 30.737 | 80.020 | 1.00 | 55.48 | C |
| ATOM | 950 | O | ALA | A | 131 | 44.913 | 30.180 | 80.176 | 1.00 | 53.95 | O |
| ATOM | 951 | N | GLN | A | 132 | 47.168 | 30.203 | 80.402 | 1.00 | 56.20 | N |
| ATOM | 952 | CA | GLN | A | 132 | 47.251 | 28.908 | 81.057 | 1.00 | 56.18 | C |
| ATOM | 953 | CB | GLN | A | 132 | 48.717 | 28.540 | 81.288 | 1.00 | 58.04 | C |
| ATOM | 954 | CG | GLN | A | 132 | 48.957 | 27.055 | 81.551 | 1.00 | 63.43 | C |
| ATOM | 955 | CD | GLN | A | 132 | 48.398 | 26.169 | 80.445 | 1.00 | 67.50 | C |
| ATOM | 956 | OE1 | GLN | A | 132 | 48.623 | 26.415 | 79.250 | 1.00 | 72.85 | O |
| ATOM | 957 | NE2 | GLN | A | 132 | 47.671 | 25.130 | 80.836 | 1.00 | 64.44 | N |
| ATOM | 958 | C | GLN | A | 132 | 46.505 | 28.958 | 82.397 | 1.00 | 55.23 | C |
| ATOM | 959 | O | GLN | A | 132 | 45.867 | 27.974 | 82.796 | 1.00 | 52.11 | O |
| ATOM | 960 | N | GLY | A | 133 | 46.572 | 30.110 | 83.071 | 1.00 | 52.14 | N |
| ATOM | 961 | CA | GLY | A | 133 | 45.893 | 30.276 | 84.346 | 1.00 | 50.42 | C |
| ATOM | 962 | C | GLY | A | 133 | 44.389 | 30.318 | 84.132 | 1.00 | 53.73 | C |
| ATOM | 963 | O | GLY | A | 133 | 43.610 | 29.895 | 84.992 | 1.00 | 54.32 | O |
| ATOM | 964 | N | VAL | A | 134 | 43.971 | 30.841 | 82.984 | 1.00 | 56.84 | N |
| ATOM | 965 | CA | VAL | A | 134 | 42.554 | 30.910 | 82.679 | 1.00 | 61.11 | C |
| ATOM | 966 | CB | VAL | A | 134 | 42.288 | 31.733 | 81.403 | 1.00 | 59.67 | C |
| ATOM | 967 | CG1 | VAL | A | 134 | 40.804 | 31.680 | 81.044 | 1.00 | 59.93 | C |
| ATOM | 968 | CG2 | VAL | A | 134 | 42.717 | 33.164 | 81.623 | 1.00 | 58.23 | C |
| ATOM | 969 | C | VAL | A | 134 | 42.007 | 29.503 | 82.492 | 1.00 | 61.64 | C |
| ATOM | 970 | O | VAL | A | 134 | 40.919 | 29.171 | 82.978 | 1.00 | 59.94 | O |
| ATOM | 971 | N | LEU | A | 135 | 42.771 | 28.676 | 81.788 | 1.00 | 62.86 | N |
| ATOM | 972 | CA | LEU | A | 135 | 42.363 | 27.307 | 81.539 | 1.00 | 65.77 | C |
| ATOM | 973 | CB | LEU | A | 135 | 43.338 | 26.645 | 80.569 | 1.00 | 69.06 | C |
| ATOM | 974 | CG | LEU | A | 135 | 42.827 | 25.348 | 79.936 | 1.00 | 73.43 | C |
| ATOM | 975 | CD1 | LEU | A | 135 | 41.353 | 25.496 | 79.522 | 1.00 | 73.08 | C |
| ATOM | 976 | CD2 | LEU | A | 135 | 43.703 | 25.004 | 78.737 | 1.00 | 74.95 | C |
| ATOM | 977 | C | LEU | A | 135 | 42.262 | 26.491 | 82.830 | 1.00 | 64.69 | C |
| ATOM | 978 | O | LEU | A | 135 | 41.329 | 25.712 | 83.005 | 1.00 | 64.30 | O |
| ATOM | 979 | N | GLU | A | 136 | 43.223 | 26.667 | 83.728 | 1.00 | 61.95 | N |
| ATOM | 980 | CA | GLU | A | 136 | 43.188 | 25.953 | 84.990 | 1.00 | 61.69 | C |
| ATOM | 981 | CB | GLU | A | 136 | 44.388 | 26.317 | 85.851 | 1.00 | 61.17 | C |
| ATOM | 982 | CG | GLU | A | 136 | 45.672 | 25.711 | 85.372 | 1.00 | 64.45 | C |
| ATOM | 983 | CD | GLU | A | 136 | 46.861 | 26.254 | 86.119 | 1.00 | 67.12 | C |
| ATOM | 984 | OE1 | GLU | A | 136 | 46.673 | 26.791 | 87.235 | 1.00 | 67.82 | O |
| ATOM | 985 | OE2 | GLU | A | 136 | 47.990 | 26.141 | 85.593 | 1.00 | 68.12 | O |
| ATOM | 986 | C | GLU | A | 136 | 41.913 | 26.308 | 85.738 | 1.00 | 61.06 | C |
| ATOM | 987 | O | GLU | A | 136 | 41.195 | 25.416 | 86.200 | 1.00 | 58.71 | O |
| ATOM | 988 | N | TYR | A | 137 | 41.632 | 27.608 | 85.845 | 1.00 | 59.24 | N |
| ATOM | 989 | CA | TYR | A | 137 | 40.450 | 28.078 | 86.547 | 1.00 | 56.86 | C |
| ATOM | 990 | CB | TYR | A | 137 | 40.349 | 29.606 | 86.467 | 1.00 | 57.82 | C |
| ATOM | 991 | CG | TYR | A | 137 | 39.238 | 30.164 | 87.327 | 1.00 | 58.56 | C |
| ATOM | 992 | CD1 | TYR | A | 137 | 37.892 | 29.995 | 86.968 | 1.00 | 58.09 | C |
| ATOM | 993 | CE1 | TYR | A | 137 | 36.866 | 30.442 | 87.788 | 1.00 | 60.04 | C |
| ATOM | 994 | CD2 | TYR | A | 137 | 39.523 | 30.804 | 88.534 | 1.00 | 57.92 | C |
| ATOM | 995 | CE2 | TYR | A | 137 | 38.499 | 31.258 | 89.367 | 1.00 | 61.29 | C |
| ATOM | 996 | CZ | TYR | A | 137 | 37.173 | 31.072 | 88.990 | 1.00 | 63.10 | C |
| ATOM | 997 | OH | TYR | A | 137 | 36.155 | 31.496 | 89.818 | 1.00 | 65.19 | O |
| ATOM | 998 | C | TYR | A | 137 | 39.182 | 27.443 | 85.972 | 1.00 | 56.62 | C |
| ATOM | 999 | O | TYR | A | 137 | 38.289 | 27.025 | 86.716 | 1.00 | 49.68 | O |
| ATOM | 1000 | N | LYS | A | 138 | 39.109 | 27.382 | 84.647 | 1.00 | 58.53 | N |
| ATOM | 1001 | CA | LYS | A | 138 | 37.953 | 26.811 | 83.964 | 1.00 | 62.44 | C |
| ATOM | 1002 | CB | LYS | A | 138 | 38.190 | 26.857 | 82.455 | 1.00 | 63.40 | C |
| ATOM | 1003 | CG | LYS | A | 138 | 37.137 | 27.601 | 81.654 | 1.00 | 68.29 | C |
| ATOM | 1004 | CD | LYS | A | 138 | 37.577 | 27.764 | 80.205 | 1.00 | 68.84 | C |
| ATOM | 1005 | CE | LYS | A | 138 | 36.504 | 27.304 | 79.232 | 1.00 | 71.45 | C |
| ATOM | 1006 | NZ | LYS | A | 138 | 36.941 | 27.408 | 77.810 | 1.00 | 73.28 | N |
| ATOM | 1007 | C | LYS | A | 138 | 37.671 | 25.369 | 84.411 | 1.00 | 63.84 | C |
| ATOM | 1008 | O | LYS | A | 138 | 36.519 | 24.998 | 84.657 | 1.00 | 60.41 | O |
| ATOM | 1009 | N | ASP | A | 139 | 38.727 | 24.565 | 84.520 | 1.00 | 64.09 | N |
| ATOM | 1010 | CA | ASP | A | 139 | 38.588 | 23.170 | 84.925 | 1.00 | 63.83 | C |
| ATOM | 1011 | CB | ASP | A | 139 | 39.718 | 22.342 | 84.302 | 1.00 | 62.60 | C |
| ATOM | 1012 | CG | ASP | A | 139 | 39.658 | 22.320 | 82.789 | 1.00 | 63.35 | C |
| ATOM | 1013 | OD1 | ASP | A | 139 | 38.542 | 22.292 | 82.242 | 1.00 | 67.93 | O |
| ATOM | 1014 | OD2 | ASP | A | 139 | 40.718 | 22.309 | 82.132 | 1.00 | 62.55 | O |
| ATOM | 1015 | C | ASP | A | 139 | 38.573 | 22.985 | 86.448 | 1.00 | 64.79 | C |
| ATOM | 1016 | O | ASP | A | 139 | 38.010 | 22.025 | 86.968 | 1.00 | 64.43 | O |
| ATOM | 1017 | N | THR | A | 140 | 39.192 | 23.920 | 87.155 | 1.00 | 65.63 | N |
| ATOM | 1018 | CA | THR | A | 140 | 39.263 | 23.869 | 88.607 | 1.00 | 68.24 | C |
| ATOM | 1019 | CB | THR | A | 140 | 40.485 | 24.660 | 89.116 | 1.00 | 70.59 | C |
| ATOM | 1020 | OG1 | THR | A | 140 | 41.674 | 24.123 | 88.529 | 1.00 | 74.84 | O |
| ATOM | 1021 | CG2 | THR | A | 140 | 40.598 | 24.558 | 90.622 | 1.00 | 75.20 | C |
| ATOM | 1022 | C | THR | A | 140 | 38.010 | 24.393 | 89.315 | 1.00 | 67.80 | C |
| ATOM | 1023 | O | THR | A | 140 | 37.640 | 23.868 | 90.366 | 1.00 | 63.87 | O |
| ATOM | 1024 | N | TYR | A | 141 | 37.361 | 25.410 | 88.745 | 1.00 | 69.77 | N |
| ATOM | 1025 | CA | TYR | A | 141 | 36.168 | 25.986 | 89.364 | 1.00 | 72.37 | C |
| ATOM | 1026 | CB | TYR | A | 141 | 36.502 | 27.333 | 90.019 | 1.00 | 72.18 | C |
| ATOM | 1027 | CG | TYR | A | 141 | 37.547 | 27.236 | 91.099 | 1.00 | 74.89 | C |
| ATOM | 1028 | CD1 | TYR | A | 141 | 38.858 | 27.638 | 90.861 | 1.00 | 74.22 | C |
| ATOM | 1029 | CE1 | TYR | A | 141 | 39.851 | 27.476 | 91.830 | 1.00 | 76.54 | C |
| ATOM | 1030 | CD2 | TYR | A | 141 | 37.243 | 26.668 | 92.341 | 1.00 | 76.71 | C |
| ATOM | 1031 | CE2 | TYR | A | 141 | 38.223 | 26.489 | 93.316 | 1.00 | 77.84 | C |
| ATOM | 1032 | CZ | TYR | A | 141 | 39.525 | 26.893 | 93.054 | 1.00 | 78.20 | C |
| ATOM | 1033 | OH | TYR | A | 141 | 40.503 | 26.690 | 94.004 | 1.00 | 80.66 | O |
| ATOM | 1034 | C | TYR | A | 141 | 34.965 | 26.176 | 88.444 | 1.00 | 74.53 | C |
| ATOM | 1035 | O | TYR | A | 141 | 33.857 | 26.443 | 88.922 | 1.00 | 73.80 | O |
| ATOM | 1036 | N | GLY | A | 142 | 35.169 | 26.058 | 87.135 | 1.00 | 76.05 | N |
| ATOM | 1037 | CA | GLY | A | 142 | 34.058 | 26.230 | 86.208 | 1.00 | 77.59 | C |
| ATOM | 1038 | C | GLY | A | 142 | 34.027 | 27.563 | 85.484 | 1.00 | 78.31 | C |
| ATOM | 1039 | O | GLY | A | 142 | 34.812 | 28.466 | 85.785 | 1.00 | 75.22 | O |
| ATOM | 1040 | N | ASP | A | 143 | 33.113 | 27.684 | 84.525 | 1.00 | 81.54 | N |
| ATOM | 1041 | CA | ASP | A | 143 | 32.986 | 28.906 | 83.743 | 1.00 | 85.20 | C |
| ATOM | 1042 | CB | ASP | A | 143 | 33.312 | 28.615 | 82.268 | 1.00 | 89.07 | C |
| ATOM | 1043 | CG | ASP | A | 143 | 32.349 | 27.616 | 81.632 | 1.00 | 94.27 | C |
| ATOM | 1044 | OD1 | ASP | A | 143 | 31.885 | 26.687 | 82.334 | 1.00 | 95.21 | O |
| ATOM | 1045 | OD2 | ASP | A | 143 | 32.073 | 27.752 | 80.420 | 1.00 | 97.10 | O |
| ATOM | 1046 | C | ASP | A | 143 | 31.611 | 29.556 | 83.876 | 1.00 | 85.68 | C |
| ATOM | 1047 | O | ASP | A | 143 | 30.753 | 29.428 | 83.004 | 1.00 | 86.47 | O |
| ATOM | 1048 | N | ASP | A | 144 | 31.417 | 30.265 | 84.981 | 1.00 | 86.66 | N |
| ATOM | 1049 | CA | ASP | A | 144 | 30.159 | 30.952 | 85.247 | 1.00 | 88.78 | C |
| ATOM | 1050 | CB | ASP | A | 144 | 29.767 | 30.763 | 86.708 | 1.00 | 90.40 | C |
| ATOM | 1051 | CG | ASP | A | 144 | 30.868 | 31.172 | 87.652 | 1.00 | 92.64 | C |
| ATOM | 1052 | OD1 | ASP | A | 144 | 31.919 | 30.495 | 87.653 | 1.00 | 93.56 | O |
| ATOM | 1053 | OD2 | ASP | A | 144 | 30.687 | 32.175 | 88.377 | 1.00 | 94.48 | O |
| ATOM | 1054 | C | ASP | A | 144 | 30.270 | 32.450 | 84.942 | 1.00 | 86.94 | C |
| ATOM | 1055 | O | ASP | A | 144 | 31.337 | 33.050 | 85.099 | 1.00 | 85.98 | O |
| ATOM | 1056 | N | PRO | A | 145 | 29.162 | 33.072 | 84.512 | 1.00 | 84.90 | N |
| ATOM | 1057 | CD | PRO | A | 145 | 27.826 | 32.472 | 84.364 | 1.00 | 84.14 | C |
| ATOM | 1058 | CA | PRO | A | 145 | 29.119 | 34.495 | 84.181 | 1.00 | 83.08 | C |
| ATOM | 1059 | CB | PRO | A | 145 | 27.626 | 34.792 | 84.181 | 1.00 | 82.70 | C |
| ATOM | 1060 | CG | PRO | A | 145 | 27.072 | 33.551 | 83.598 | 1.00 | 83.96 | C |
| ATOM | 1061 | C | PRO | A | 145 | 29.892 | 35.396 | 85.133 | 1.00 | 81.13 | C |
| ATOM | 1062 | O | PRO | A | 145 | 30.545 | 36.347 | 84.706 | 1.00 | 81.70 | O |
| ATOM | 1063 | N | VAL | A | 146 | 29.823 | 35.102 | 86.424 | 1.00 | 78.41 | N |
| ATOM | 1064 | CA | VAL | A | 146 | 30.521 | 35.926 | 87.399 | 1.00 | 77.84 | C |
| ATOM | 1065 | CB | VAL | A | 146 | 30.068 | 35.600 | 88.838 | 1.00 | 77.87 | C |
| ATOM | 1066 | CG1 | VAL | A | 146 | 30.864 | 36.432 | 89.844 | 1.00 | 74.64 | C |
| ATOM | 1067 | CG2 | VAL | A | 146 | 28.589 | 35.893 | 88.983 | 1.00 | 77.65 | C |
| ATOM | 1068 | C | VAL | A | 146 | 32.034 | 35.801 | 87.314 | 1.00 | 77.09 | C |
| ATOM | 1069 | O | VAL | A | 146 | 32.733 | 36.805 | 87.237 | 1.00 | 76.58 | O |
| ATOM | 1070 | N | SER | A | 147 | 32.547 | 34.577 | 87.336 | 1.00 | 76.40 | N |
| ATOM | 1071 | CA | SER | A | 147 | 33.989 | 34.389 | 87.257 | 1.00 | 76.60 | C |
| ATOM | 1072 | CB | SER | A | 147 | 34.351 | 32.910 | 87.426 | 1.00 | 76.75 | C |
| ATOM | 1073 | OG | SER | A | 147 | 33.829 | 32.115 | 86.383 | 1.00 | 78.76 | O |
| ATOM | 1074 | C | SER | A | 147 | 34.511 | 34.922 | 85.920 | 1.00 | 75.20 | C |
| ATOM | 1075 | O | SER | A | 147 | 35.650 | 35.376 | 85.824 | 1.00 | 74.57 | O |
| ATOM | 1076 | N | ASN | A | 148 | 33.667 | 34.874 | 84.894 | 1.00 | 72.89 | N |
| ATOM | 1077 | CA | ASN | A | 148 | 34.038 | 35.368 | 83.576 | 1.00 | 73.96 | C |
| ATOM | 1078 | CB | ASN | A | 148 | 32.989 | 34.957 | 82.547 | 1.00 | 75.49 | C |
| ATOM | 1079 | CG | ASN | A | 148 | 33.245 | 35.573 | 81.196 | 1.00 | 78.27 | C |
| ATOM | 1080 | OD1 | ASN | A | 148 | 34.204 | 35.221 | 80.516 | 1.00 | 78.59 | O |
| ATOM | 1081 | ND2 | ASN | A | 148 | 32.397 | 36.516 | 80.805 | 1.00 | 80.82 | N |
| ATOM | 1082 | C | ASN | A | 148 | 34.163 | 36.894 | 83.578 | 1.00 | 73.86 | C |
| ATOM | 1083 | O | ASN | A | 148 | 35.100 | 37.453 | 83.015 | 1.00 | 74.07 | O |
| ATOM | 1084 | N | GLN | A | 149 | 33.198 | 37.554 | 84.205 | 1.00 | 73.28 | N |
| ATOM | 1085 | CA | GLN | A | 149 | 33.171 | 39.002 | 84.292 | 1.00 | 73.47 | C |
| ATOM | 1086 | CB | GLN | A | 149 | 31.895 | 39.452 | 84.999 | 1.00 | 77.22 | C |
| ATOM | 1087 | CG | GLN | A | 149 | 31.280 | 40.722 | 84.440 | 1.00 | 84.71 | C |
| ATOM | 1088 | CD | GLN | A | 149 | 30.926 | 40.587 | 82.969 | 1.00 | 90.76 | C |
| ATOM | 1089 | OE1 | GLN | A | 149 | 31.635 | 41.097 | 82.090 | 1.00 | 93.22 | O |
| ATOM | 1090 | NE2 | GLN | A | 149 | 29.833 | 39.881 | 82.690 | 1.00 | 92.08 | N |
| ATOM | 1091 | C | GLN | A | 149 | 34.385 | 39.501 | 85.068 | 1.00 | 71.91 | C |
| ATOM | 1092 | O | GLN | A | 149 | 34.987 | 40.510 | 84.715 | 1.00 | 73.73 | O |
| ATOM | 1093 | N | ASN | A | 150 | 34.738 | 38.792 | 86.134 | 1.00 | 69.45 | N |
| ATOM | 1094 | CA | ASN | A | 150 | 35.883 | 39.163 | 86.957 | 1.00 | 68.31 | C |
| ATOM | 1095 | CB | ASN | A | 150 | 35.915 | 38.320 | 88.232 | 1.00 | 74.21 | C |
| ATOM | 1096 | CG | ASN | A | 150 | 34.814 | 38.699 | 89.201 | 1.00 | 79.60 | C |
| ATOM | 1097 | OD1 | ASN | A | 150 | 34.718 | 39.850 | 89.619 | 1.00 | 81.23 | O |
| ATOM | 1098 | ND2 | ASN | A | 150 | 33.975 | 37.732 | 89.563 | 1.00 | 84.47 | N |
| ATOM | 1099 | C | ASN | A | 150 | 37.184 | 39.005 | 86.198 | 1.00 | 64.06 | C |
| ATOM | 1100 | O | ASN | A | 150 | 38.019 | 39.902 | 86.201 | 1.00 | 62.78 | O |
| ATOM | 1101 | N | ILE | A | 151 | 37.354 | 37.859 | 85.553 | 1.00 | 59.14 | N |
| ATOM | 1102 | CA | ILE | A | 151 | 38.553 | 37.596 | 84.766 | 1.00 | 56.37 | C |
| ATOM | 1103 | CB | ILE | A | 151 | 38.513 | 36.159 | 84.166 | 1.00 | 54.51 | C |
| ATOM | 1104 | CG2 | ILE | A | 151 | 39.713 | 35.927 | 83.247 | 1.00 | 46.33 | C |
| ATOM | 1105 | CG1 | ILE | A | 151 | 38.493 | 35.134 | 85.305 | 1.00 | 56.38 | C |
| ATOM | 1106 | CD1 | ILE | A | 151 | 38.280 | 33.708 | 84.843 | 1.00 | 55.94 | C |
| ATOM | 1107 | C | ILE | A | 151 | 38.696 | 38.619 | 83.631 | 1.00 | 54.83 | C |
| ATOM | 1108 | O | ILE | A | 151 | 39.785 | 39.148 | 83.386 | 1.00 | 49.38 | O |
| ATOM | 1109 | N | GLN | A | 152 | 37.581 | 38.873 | 82.947 | 1.00 | 54.48 | N |
| ATOM | 1110 | CA | GLN | A | 152 | 37.525 | 39.819 | 81.834 | 1.00 | 56.99 | C |
| ATOM | 1111 | CB | GLN | A | 152 | 36.107 | 39.896 | 81.291 | 1.00 | 59.58 | C |
| ATOM | 1112 | CG | GLN | A | 152 | 35.969 | 40.642 | 79.986 | 1.00 | 62.52 | C |
| ATOM | 1113 | CD | GLN | A | 152 | 36.620 | 39.903 | 78.840 | 1.00 | 66.18 | C |
| ATOM | 1114 | OE1 | GLN | A | 152 | 36.668 | 38.669 | 78.830 | 1.00 | 61.84 | O |
| ATOM | 1115 | NE2 | GLN | A | 152 | 37.112 | 40.653 | 77.855 | 1.00 | 65.01 | N |
| ATOM | 1116 | C | GLN | A | 152 | 37.938 | 41.208 | 82.298 | 1.00 | 56.05 | C |
| ATOM | 1117 | O | GLN | A | 152 | 38.726 | 41.889 | 81.653 | 1.00 | 51.54 | O |
| ATOM | 1118 | N | TYR | A | 153 | 37.382 | 41.613 | 83.434 | 1.00 | 55.19 | N |
| ATOM | 1119 | CA | TYR | A | 153 | 37.675 | 42.908 | 84.033 | 1.00 | 56.71 | C |
| ATOM | 1120 | CB | TYR | A | 153 | 36.886 | 43.085 | 85.332 | 1.00 | 59.74 | C |
| ATOM | 1121 | CG | TYR | A | 153 | 37.160 | 44.400 | 86.036 | 1.00 | 67.79 | C |
| ATOM | 1122 | CD1 | TYR | A | 153 | 36.525 | 45.582 | 85.632 | 1.00 | 72.26 | C |
| ATOM | 1123 | CE1 | TYR | A | 153 | 36.799 | 46.804 | 86.259 | 1.00 | 72.87 | C |
| ATOM | 1124 | CD2 | TYR | A | 153 | 38.080 | 44.471 | 87.090 | 1.00 | 70.33 | C |
| ATOM | 1125 | CE2 | TYR | A | 153 | 38.365 | 45.687 | 87.727 | 1.00 | 72.78 | C |
| ATOM | 1126 | CZ | TYR | A | 153 | 37.721 | 46.850 | 87.305 | 1.00 | 75.11 | C |
| ATOM | 1127 | OH | TYR | A | 153 | 38.003 | 48.052 | 87.923 | 1.00 | 77.60 | O |
| ATOM | 1128 | C | TYR | A | 153 | 39.159 | 43.012 | 84.348 | 1.00 | 54.89 | C |
| ATOM | 1129 | O | TYR | A | 153 | 39.819 | 43.980 | 83.989 | 1.00 | 54.26 | O |
| ATOM | 1130 | N | PHE | A | 154 | 39.684 | 41.994 | 85.017 | 1.00 | 53.12 | N |
| ATOM | 1131 | CA | PHE | A | 154 | 41.089 | 41.973 | 85.397 | 1.00 | 49.90 | C |
| ATOM | 1132 | CB | PHE | A | 154 | 41.385 | 40.788 | 86.337 | 1.00 | 49.72 | C |
| ATOM | 1133 | CG | PHE | A | 154 | 42.863 | 40.552 | 86.554 | 1.00 | 48.78 | C |
| ATOM | 1134 | CD1 | PHE | A | 154 | 43.528 | 39.523 | 85.875 | 1.00 | 47.55 | C |
| ATOM | 1135 | CD2 | PHE | A | 154 | 43.612 | 41.420 | 87.354 | 1.00 | 44.82 | C |
| ATOM | 1136 | CE1 | PHE | A | 154 | 44.914 | 39.368 | 85.985 | 1.00 | 46.64 | C |
| ATOM | 1137 | CE2 | PHE | A | 154 | 44.991 | 41.277 | 87.473 | 1.00 | 46.40 | C |
| ATOM | 1138 | CZ | PHE | A | 154 | 45.651 | 40.254 | 86.790 | 1.00 | 46.27 | C |
| ATOM | 1139 | C | PHE | A | 154 | 42.049 | 41.939 | 84.211 | 1.00 | 48.64 | C |
| ATOM | 1140 | O | PHE | A | 154 | 42.967 | 42.744 | 84.134 | 1.00 | 45.06 | O |
| ATOM | 1141 | N | LEU | A | 155 | 41.832 | 41.009 | 83.289 | 1.00 | 49.13 | N |
| ATOM | 1142 | CA | LEU | A | 155 | 42.703 | 40.889 | 82.131 | 1.00 | 47.03 | C |
| ATOM | 1143 | CB | LEU | A | 155 | 42.343 | 39.630 | 81.330 | 1.00 | 47.52 | C |
| ATOM | 1144 | CG | LEU | A | 155 | 42.671 | 38.311 | 82.053 | 1.00 | 47.91 | C |
| ATOM | 1145 | CD1 | LEU | A | 155 | 42.357 | 37.147 | 81.160 | 1.00 | 41.41 | C |
| ATOM | 1146 | CD2 | LEU | A | 155 | 44.146 | 38.290 | 82.438 | 1.00 | 46.13 | C |
| ATOM | 1147 | C | LEU | A | 155 | 42.714 | 42.124 | 81.232 | 1.00 | 44.12 | C |
| ATOM | 1148 | O | LEU | A | 155 | 43.783 | 42.534 | 80.764 | 1.00 | 39.93 | O |
| ATOM | 1149 | N | ASP | A | 156 | 41.550 | 42.726 | 81.005 | 1.00 | 39.62 | N |
| ATOM | 1150 | CA | ASP | A | 156 | 41.491 | 43.915 | 80.169 | 1.00 | 42.54 | C |
| ATOM | 1151 | CB | ASP | A | 156 | 40.051 | 44.446 | 80.035 | 1.00 | 42.46 | C |
| ATOM | 1152 | CG | ASP | A | 156 | 39.294 | 43.801 | 78.900 | 1.00 | 44.44 | C |
| ATOM | 1153 | OD1 | ASP | A | 156 | 39.923 | 43.266 | 77.976 | 1.00 | 44.53 | O |
| ATOM | 1154 | OD2 | ASP | A | 156 | 38.065 | 43.850 | 78.920 | 1.00 | 46.51 | O |
| ATOM | 1155 | C | ASP | A | 156 | 42.385 | 45.014 | 80.740 | 1.00 | 43.07 | C |
| ATOM | 1156 | O | ASP | A | 156 | 43.091 | 45.696 | 79.985 | 1.00 | 40.86 | O |
| ATOM | 1157 | N | ARG | A | 157 | 42.355 | 45.188 | 82.063 | 1.00 | 43.86 | N |
| ATOM | 1158 | CA | ARG | A | 157 | 43.170 | 46.218 | 82.709 | 1.00 | 45.40 | C |
| ATOM | 1159 | CB | ARG | A | 157 | 42.661 | 46.484 | 84.130 | 1.00 | 45.43 | C |
| ATOM | 1160 | CG | ARG | A | 157 | 41.249 | 47.078 | 84.187 | 1.00 | 55.59 | C |
| ATOM | 1161 | CD | ARG | A | 157 | 40.653 | 46.969 | 85.593 | 1.00 | 57.67 | C |
| ATOM | 1162 | NE | ARG | A | 157 | 41.436 | 47.636 | 86.633 | 1.00 | 64.11 | N |
| ATOM | 1163 | CZ | ARG | A | 157 | 41.386 | 48.935 | 86.905 | 1.00 | 65.60 | C |
| ATOM | 1164 | NH1 | ARG | A | 157 | 40.582 | 49.734 | 86.220 | 1.00 | 68.57 | N |
| ATOM | 1165 | NH2 | ARG | A | 157 | 42.158 | 49.435 | 87.856 | 1.00 | 63.78 | N |
| ATOM | 1166 | C | ARG | A | 157 | 44.644 | 45.826 | 82.746 | 1.00 | 46.09 | C |
| ATOM | 1167 | O | ARG | A | 157 | 45.517 | 46.645 | 82.445 | 1.00 | 47.23 | O |
| ATOM | 1168 | N | PHE | A | 158 | 44.906 | 44.572 | 83.110 | 1.00 | 46.47 | N |
| ATOM | 1169 | CA | PHE | A | 158 | 46.258 | 44.046 | 83.189 | 1.00 | 44.00 | C |
| ATOM | 1170 | CB | PHE | A | 158 | 46.224 | 42.542 | 83.526 | 1.00 | 50.46 | C |
| ATOM | 1171 | CG | PHE | A | 158 | 47.581 | 41.886 | 83.482 | 1.00 | 52.69 | C |
| ATOM | 1172 | CD1 | PHE | A | 158 | 48.540 | 42.168 | 84.456 | 1.00 | 53.47 | C |
| ATOM | 1173 | CD2 | PHE | A | 158 | 47.929 | 41.065 | 82.421 | 1.00 | 55.28 | C |
| ATOM | 1174 | CE1 | PHE | A | 158 | 49.826 | 41.650 | 84.373 | 1.00 | 54.61 | C |
| ATOM | 1175 | CE2 | PHE | A | 158 | 49.224 | 40.533 | 82.319 | 1.00 | 59.41 | C |
| ATOM | 1176 | CZ | PHE | A | 158 | 50.179 | 40.829 | 83.301 | 1.00 | 56.08 | C |
| ATOM | 1177 | C | PHE | A | 158 | 46.979 | 44.257 | 81.858 | 1.00 | 45.59 | C |
| ATOM | 1178 | O | PHE | A | 158 | 48.072 | 44.857 | 81.809 | 1.00 | 42.64 | O |
| ATOM | 1179 | N | TYR | A | 159 | 46.343 | 43.780 | 80.782 | 1.00 | 42.87 | N |
| ATOM | 1180 | CA | TYR | A | 159 | 46.903 | 43.870 | 79.440 | 1.00 | 42.18 | C |
| ATOM | 1181 | CB | TYR | A | 159 | 46.085 | 43.000 | 78.481 | 1.00 | 48.47 | C |
| ATOM | 1182 | CG | TYR | A | 159 | 46.315 | 41.505 | 78.634 | 1.00 | 54.66 | C |
| ATOM | 1183 | CD1 | TYR | A | 159 | 47.611 | 40.980 | 78.721 | 1.00 | 58.46 | C |
| ATOM | 1184 | CE1 | TYR | A | 159 | 47.827 | 39.605 | 78.819 | 1.00 | 59.30 | C |
| ATOM | 1185 | CD2 | TYR | A | 159 | 45.240 | 40.608 | 78.653 | 1.00 | 55.47 | C |
| ATOM | 1186 | CE2 | TYR | A | 159 | 45.448 | 39.230 | 78.749 | 1.00 | 56.07 | C |
| ATOM | 1187 | CZ | TYR | A | 159 | 46.741 | 38.737 | 78.828 | 1.00 | 60.06 | C |
| ATOM | 1188 | OH | TYR | A | 159 | 46.958 | 37.378 | 78.884 | 1.00 | 56.35 | O |
| ATOM | 1189 | C | TYR | A | 159 | 47.031 | 45.294 | 78.888 | 1.00 | 41.71 | C |
| ATOM | 1190 | O | TYR | A | 159 | 47.985 | 45.591 | 78.190 | 1.00 | 37.93 | O |
| ATOM | 1191 | N | LEU | A | 160 | 46.083 | 46.174 | 79.202 | 1.00 | 40.01 | N |
| ATOM | 1192 | CA | LEU | A | 160 | 46.161 | 47.556 | 78.725 | 1.00 | 44.47 | C |
| ATOM | 1193 | CB | LEU | A | 160 | 44.926 | 48.358 | 79.144 | 1.00 | 44.60 | C |
| ATOM | 1194 | CG | LEU | A | 160 | 44.208 | 49.249 | 78.109 | 1.00 | 48.16 | C |
| ATOM | 1195 | CD1 | LEU | A | 160 | 43.400 | 50.280 | 78.889 | 1.00 | 45.21 | C |
| ATOM | 1196 | CD2 | LEU | A | 160 | 45.180 | 49.941 | 77.164 | 1.00 | 40.90 | C |
| ATOM | 1197 | C | LEU | A | 160 | 47.402 | 48.186 | 79.354 | 1.00 | 44.29 | C |
| ATOM | 1198 | O | LEU | A | 160 | 48.206 | 48.853 | 78.688 | 1.00 | 39.99 | O |
| ATOM | 1199 | N | SER | A | 161 | 47.546 | 47.954 | 80.654 | 1.00 | 45.64 | N |
| ATOM | 1200 | CA | SER | A | 161 | 48.686 | 48.455 | 81.416 | 1.00 | 49.12 | C |
| ATOM | 1201 | CB | SER | A | 161 | 48.583 | 47.951 | 82.862 | 1.00 | 51.81 | C |
| ATOM | 1202 | OG | SER | A | 161 | 49.854 | 47.853 | 83.484 | 1.00 | 62.66 | O |
| ATOM | 1203 | C | SER | A | 161 | 49.977 | 47.975 | 80.745 | 1.00 | 47.35 | C |
| ATOM | 1204 | O | SER | A | 161 | 50.865 | 48.763 | 80.477 | 1.00 | 44.49 | O |
| ATOM | 1205 | N | ARG | A | 162 | 50.042 | 46.685 | 80.427 | 1.00 | 45.01 | N |
| ATOM | 1206 | CA | ARG | A | 162 | 51.217 | 46.122 | 79.776 | 1.00 | 46.40 | C |
| ATOM | 1207 | CB | ARG | A | 162 | 51.036 | 44.608 | 79.557 | 1.00 | 46.19 | C |
| ATOM | 1208 | CG | ARG | A | 162 | 52.207 | 43.937 | 78.873 | 1.00 | 47.42 | C |
| ATOM | 1209 | CD | ARG | A | 162 | 52.060 | 42.428 | 78.738 | 1.00 | 52.24 | C |
| ATOM | 1210 | NE | ARG | A | 162 | 53.253 | 41.859 | 78.091 | 1.00 | 55.81 | N |
| ATOM | 1211 | CZ | ARG | A | 162 | 53.284 | 41.418 | 76.838 | 1.00 | 61.66 | C |
| ATOM | 1212 | NH1 | ARG | A | 162 | 52.181 | 41.465 | 76.100 | 1.00 | 64.69 | N |
| ATOM | 1213 | NH2 | ARG | A | 162 | 54.419 | 40.969 | 76.315 | 1.00 | 65.87 | N |
| ATOM | 1214 | C | ARG | A | 162 | 51.513 | 46.829 | 78.448 | 1.00 | 46.46 | C |
| ATOM | 1215 | O | ARG | A | 162 | 52.674 | 47.130 | 78.131 | 1.00 | 43.85 | O |
| ATOM | 1216 | N | ILE | A | 163 | 50.467 | 47.094 | 77.668 | 1.00 | 44.84 | N |
| ATOM | 1217 | CA | ILE | A | 163 | 50.631 | 47.758 | 76.377 | 1.00 | 40.39 | C |
| ATOM | 1218 | CB | ILE | A | 163 | 49.254 | 47.895 | 75.623 | 1.00 | 44.28 | C |
| ATOM | 1219 | CG2 | ILE | A | 163 | 49.401 | 48.809 | 74.374 | 1.00 | 34.70 | C |
| ATOM | 1220 | CG1 | ILE | A | 163 | 48.736 | 46.501 | 75.240 | 1.00 | 42.82 | C |
| ATOM | 1221 | CD1 | ILE | A | 163 | 47.352 | 46.481 | 74.566 | 1.00 | 44.21 | C |
| ATOM | 1222 | C | ILE | A | 163 | 51.263 | 49.141 | 76.569 | 1.00 | 41.51 | C |
| ATOM | 1223 | O | ILE | A | 163 | 52.181 | 49.525 | 75.834 | 1.00 | 39.41 | O |
| ATOM | 1224 | N | SER | A | 164 | 50.770 | 49.886 | 77.560 | 1.00 | 39.51 | N |
| ATOM | 1225 | CA | SER | A | 164 | 51.278 | 51.234 | 77.858 | 1.00 | 43.36 | C |
| ATOM | 1226 | CB | SER | A | 164 | 50.443 | 51.887 | 78.968 | 1.00 | 41.28 | C |
| ATOM | 1227 | OG | SER | A | 164 | 50.877 | 51.458 | 80.236 | 1.00 | 44.13 | O |
| ATOM | 1228 | C | SER | A | 164 | 52.748 | 51.246 | 78.288 | 1.00 | 46.74 | C |
| ATOM | 1229 | O | SER | A | 164 | 53.509 | 52.153 | 77.930 | 1.00 | 44.40 | O |
| ATOM | 1230 | N | ILE | A | 165 | 53.127 | 50.248 | 79.085 | 1.00 | 46.50 | N |
| ATOM | 1231 | CA | ILE | A | 165 | 54.490 | 50.133 | 79.577 | 1.00 | 44.47 | C |
| ATOM | 1232 | CB | ILE | A | 165 | 54.574 | 49.076 | 80.678 | 1.00 | 43.70 | C |
| ATOM | 1233 | CG2 | ILE | A | 165 | 56.025 | 48.708 | 80.966 | 1.00 | 41.26 | C |
| ATOM | 1234 | CG1 | ILE | A | 165 | 53.878 | 49.608 | 81.924 | 1.00 | 42.60 | C |
| ATOM | 1235 | CD1 | ILE | A | 165 | 53.674 | 48.573 | 83.008 | 1.00 | 46.54 | C |
| ATOM | 1236 | C | ILE | A | 165 | 55.419 | 49.785 | 78.434 | 1.00 | 46.49 | C |
| ATOM | 1237 | O | ILE | A | 165 | 56.496 | 50.373 | 78.306 | 1.00 | 46.80 | O |
| ATOM | 1238 | N | ARG | A | 166 | 54.996 | 48.857 | 77.583 | 1.00 | 48.42 | N |
| ATOM | 1239 | CA | ARG | A | 166 | 55.823 | 48.475 | 76.453 | 1.00 | 51.04 | C |
| ATOM | 1240 | CB | ARG | A | 166 | 55.239 | 47.238 | 75.774 | 1.00 | 55.50 | C |
| ATOM | 1241 | CG | ARG | A | 166 | 55.484 | 45.935 | 76.572 | 1.00 | 64.25 | C |
| ATOM | 1242 | CD | ARG | A | 166 | 54.604 | 44.787 | 76.088 | 1.00 | 68.88 | C |
| ATOM | 1243 | NE | ARG | A | 166 | 54.781 | 44.526 | 74.660 | 1.00 | 75.66 | N |
| ATOM | 1244 | CZ | ARG | A | 166 | 55.849 | 43.937 | 74.134 | 1.00 | 80.29 | C |
| ATOM | 1245 | NH1 | ARG | A | 166 | 56.847 | 43.532 | 74.922 | 1.00 | 80.85 | N |
| ATOM | 1246 | NH2 | ARG | A | 166 | 55.924 | 43.778 | 72.817 | 1.00 | 82.04 | N |
| ATOM | 1247 | C | ARG | A | 166 | 55.988 | 49.627 | 75.468 | 1.00 | 50.68 | C |
| ATOM | 1248 | O | ARG | A | 166 | 56.989 | 49.733 | 74.792 | 1.00 | 48.99 | O |
| ATOM | 1249 | N | MET | A | 167 | 55.001 | 50.510 | 75.419 | 1.00 | 51.44 | N |
| ATOM | 1250 | CA | MET | A | 167 | 55.044 | 51.676 | 74.540 | 1.00 | 52.19 | C |
| ATOM | 1251 | CB | MET | A | 167 | 53.677 | 52.403 | 74.546 | 1.00 | 54.36 | C |
| ATOM | 1252 | CG | MET | A | 167 | 53.592 | 53.640 | 73.665 | 1.00 | 54.86 | C |
| ATOM | 1253 | SD | MET | A | 167 | 53.844 | 53.319 | 71.882 | 1.00 | 62.12 | S |
| ATOM | 1254 | CE | MET | A | 167 | 54.079 | 54.999 | 71.254 | 1.00 | 58.47 | C |
| ATOM | 1255 | C | MET | A | 167 | 56.158 | 52.614 | 75.031 | 1.00 | 52.84 | C |
| ATOM | 1256 | O | MET | A | 167 | 56.973 | 53.094 | 74.238 | 1.00 | 49.60 | O |
| ATOM | 1257 | N | LEU | A | 168 | 56.188 | 52.871 | 76.338 | 1.00 | 50.02 | N |
| ATOM | 1258 | CA | LEU | A | 168 | 57.207 | 53.742 | 76.900 | 1.00 | 52.20 | C |
| ATOM | 1259 | CB | LEU | A | 168 | 57.009 | 53.918 | 78.411 | 1.00 | 49.38 | C |
| ATOM | 1260 | CG | LEU | A | 168 | 55.668 | 54.528 | 78.869 | 1.00 | 52.91 | C |
| ATOM | 1261 | CD1 | LEU | A | 168 | 55.761 | 54.974 | 80.323 | 1.00 | 41.22 | C |
| ATOM | 1262 | CD2 | LEU | A | 168 | 55.312 | 55.710 | 77.981 | 1.00 | 48.62 | C |
| ATOM | 1263 | C | LEU | A | 168 | 58.584 | 53.157 | 76.623 | 1.00 | 53.56 | C |
| ATOM | 1264 | O | LEU | A | 168 | 59.480 | 53.845 | 76.122 | 1.00 | 51.75 | O |
| ATOM | 1265 | N | ILE | A | 169 | 58.751 | 51.873 | 76.916 | 1.00 | 54.05 | N |
| ATOM | 1266 | CA | ILE | A | 169 | 60.043 | 51.232 | 76.699 | 1.00 | 54.92 | C |
| ATOM | 1267 | CB | ILE | A | 169 | 60.054 | 49.808 | 77.265 | 1.00 | 54.94 | C |
| ATOM | 1268 | CG2 | ILE | A | 169 | 61.292 | 49.060 | 76.778 | 1.00 | 44.91 | C |
| ATOM | 1269 | CG1 | ILE | A | 169 | 59.984 | 49.864 | 78.795 | 1.00 | 54.86 | C |
| ATOM | 1270 | CD1 | ILE | A | 169 | 59.994 | 48.518 | 79.451 | 1.00 | 57.45 | C |
| ATOM | 1271 | C | ILE | A | 169 | 60.497 | 51.178 | 75.246 | 1.00 | 57.21 | C |
| ATOM | 1272 | O | ILE | A | 169 | 61.677 | 51.357 | 74.964 | 1.00 | 54.12 | O |
| ATOM | 1273 | N | ASN | A | 170 | 59.575 | 50.923 | 74.324 | 1.00 | 59.72 | N |
| ATOM | 1274 | CA | ASN | A | 170 | 59.947 | 50.862 | 72.919 | 1.00 | 63.46 | C |
| ATOM | 1275 | CB | ASN | A | 170 | 58.808 | 50.288 | 72.066 | 1.00 | 68.20 | C |
| ATOM | 1276 | CG | ASN | A | 170 | 58.611 | 48.789 | 72.279 | 1.00 | 73.19 | C |
| ATOM | 1277 | OD1 | ASN | A | 170 | 59.579 | 48.032 | 72.424 | 1.00 | 74.08 | O |
| ATOM | 1278 | ND2 | ASN | A | 170 | 57.351 | 48.354 | 72.294 | 1.00 | 73.91 | N |
| ATOM | 1279 | C | ASN | A | 170 | 60.335 | 52.240 | 72.406 | 1.00 | 64.38 | C |
| ATOM | 1280 | O | ASN | A | 170 | 61.276 | 52.371 | 71.625 | 1.00 | 63.69 | O |
| ATOM | 1281 | N | GLN | A | 171 | 59.616 | 53.268 | 72.853 | 1.00 | 64.33 | N |
| ATOM | 1282 | CA | GLN | A | 171 | 59.915 | 54.635 | 72.434 | 1.00 | 66.38 | C |
| ATOM | 1283 | CB | GLN | A | 171 | 58.898 | 55.626 | 73.021 | 1.00 | 65.82 | C |
| ATOM | 1284 | CG | GLN | A | 171 | 57.615 | 55.795 | 72.196 | 1.00 | 66.80 | C |
| ATOM | 1285 | CD | GLN | A | 171 | 57.888 | 56.081 | 70.715 | 1.00 | 68.40 | C |
| ATOM | 1286 | OE1 | GLN | A | 171 | 58.078 | 55.165 | 69.912 | 1.00 | 61.62 | O |
| ATOM | 1287 | NE2 | GLN | A | 171 | 57.914 | 57.360 | 70.356 | 1.00 | 68.24 | N |
| ATOM | 1288 | C | GLN | A | 171 | 61.323 | 55.022 | 72.875 | 1.00 | 67.71 | C |
| ATOM | 1289 | O | GLN | A | 171 | 62.067 | 55.643 | 72.124 | 1.00 | 65.84 | O |
| ATOM | 1290 | N | HIS | A | 172 | 61.693 | 54.641 | 74.092 | 1.00 | 68.86 | N |
| ATOM | 1291 | CA | HIS | A | 172 | 63.017 | 54.962 | 74.598 | 1.00 | 71.88 | C |
| ATOM | 1292 | CB | HIS | A | 172 | 63.094 | 54.689 | 76.096 | 1.00 | 73.33 | C |
| ATOM | 1293 | CG | HIS | A | 172 | 64.330 | 55.230 | 76.737 | 1.00 | 78.71 | C |
| ATOM | 1294 | CD2 | HIS | A | 172 | 65.553 | 54.676 | 76.924 | 1.00 | 80.45 | C |
| ATOM | 1295 | ND1 | HIS | A | 172 | 64.416 | 56.517 | 77.224 | 1.00 | 79.61 | N |
| ATOM | 1296 | CE1 | HIS | A | 172 | 65.635 | 56.732 | 77.684 | 1.00 | 80.02 | C |
| ATOM | 1297 | NE2 | HIS | A | 172 | 66.345 | 55.631 | 77.512 | 1.00 | 81.09 | N |
| ATOM | 1298 | C | HIS | A | 172 | 64.126 | 54.177 | 73.894 | 1.00 | 72.89 | C |
| ATOM | 1299 | O | HIS | A | 172 | 65.079 | 54.751 | 73.385 | 1.00 | 71.57 | O |
| ATOM | 1300 | N | THR | A | 173 | 64.005 | 52.858 | 73.872 | 1.00 | 74.39 | N |
| ATOM | 1301 | CA | THR | A | 173 | 65.016 | 52.020 | 73.241 | 1.00 | 76.60 | C |
| ATOM | 1302 | CB | THR | A | 173 | 64.689 | 50.520 | 73.398 | 1.00 | 77.59 | C |
| ATOM | 1303 | OG1 | THR | A | 173 | 63.429 | 50.230 | 72.774 | 1.00 | 80.50 | O |
| ATOM | 1304 | CG2 | THR | A | 173 | 64.636 | 50.146 | 74.878 | 1.00 | 75.27 | C |
| ATOM | 1305 | C | THR | A | 173 | 65.195 | 52.317 | 71.759 | 1.00 | 77.83 | C |
| ATOM | 1306 | O | THR | A | 173 | 66.315 | 52.410 | 71.278 | 1.00 | 78.82 | O |
| ATOM | 1307 | N | LEU | A | 174 | 64.097 | 52.461 | 71.030 | 1.00 | 79.41 | N |
| ATOM | 1308 | CA | LEU | A | 174 | 64.180 | 52.742 | 69.598 | 1.00 | 79.96 | C |
| ATOM | 1309 | CB | LEU | A | 174 | 62.789 | 52.679 | 68.960 | 1.00 | 79.55 | C |
| ATOM | 1310 | CG | LEU | A | 174 | 62.125 | 51.314 | 68.784 | 1.00 | 78.37 | C |
| ATOM | 1311 | CD1 | LEU | A | 174 | 60.717 | 51.515 | 68.254 | 1.00 | 80.11 | C |
| ATOM | 1312 | CD2 | LEU | A | 174 | 62.935 | 50.456 | 67.829 | 1.00 | 72.44 | C |
| ATOM | 1313 | C | LEU | A | 174 | 64.797 | 54.110 | 69.308 | 1.00 | 82.16 | C |
| ATOM | 1314 | O | LEU | A | 174 | 65.656 | 54.238 | 68.435 | 1.00 | 80.69 | O |
| ATOM | 1315 | N | ILE | A | 175 | 64.356 | 55.124 | 70.049 | 1.00 | 84.50 | N |
| ATOM | 1316 | CA | ILE | A | 175 | 64.835 | 56.491 | 69.874 | 1.00 | 86.92 | C |
| ATOM | 1317 | CB | ILE | A | 175 | 63.933 | 57.479 | 70.664 | 1.00 | 87.66 | C |
| ATOM | 1318 | CG2 | ILE | A | 175 | 64.576 | 58.859 | 70.728 | 1.00 | 86.66 | C |
| ATOM | 1319 | CG1 | ILE | A | 175 | 62.553 | 57.549 | 69.996 | 1.00 | 87.88 | C |
| ATOM | 1320 | CD1 | ILE | A | 175 | 61.550 | 58.443 | 70.700 | 1.00 | 86.97 | C |
| ATOM | 1321 | C | ILE | A | 175 | 66.305 | 56.706 | 70.254 | 1.00 | 88.77 | C |
| ATOM | 1322 | O | ILE | A | 175 | 67.009 | 57.491 | 69.620 | 1.00 | 87.31 | O |
| ATOM | 1323 | N | PHE | A | 176 | 66.778 | 55.994 | 71.271 | 1.00 | 90.24 | N |
| ATOM | 1324 | CA | PHE | A | 176 | 68.164 | 56.144 | 71.704 | 1.00 | 92.25 | C |
| ATOM | 1325 | CB | PHE | A | 176 | 68.181 | 56.473 | 73.193 | 1.00 | 88.92 | C |
| ATOM | 1326 | CG | PHE | A | 176 | 67.275 | 57.614 | 73.545 | 1.00 | 85.93 | C |
| ATOM | 1327 | CD1 | PHE | A | 176 | 67.487 | 58.875 | 73.008 | 1.00 | 83.63 | C |
| ATOM | 1328 | CD2 | PHE | A | 176 | 66.177 | 57.418 | 74.366 | 1.00 | 86.06 | C |
| ATOM | 1329 | CE1 | PHE | A | 176 | 66.617 | 59.922 | 73.282 | 1.00 | 83.20 | C |
| ATOM | 1330 | CE2 | PHE | A | 176 | 65.293 | 58.458 | 74.650 | 1.00 | 83.21 | C |
| ATOM | 1331 | CZ | PHE | A | 176 | 65.515 | 59.714 | 74.105 | 1.00 | 82.18 | C |
| ATOM | 1332 | C | PHE | A | 176 | 69.033 | 54.932 | 71.392 | 1.00 | 96.40 | C |
| ATOM | 1333 | O | PHE | A | 176 | 69.772 | 54.441 | 72.250 | 1.00 | 95.67 | O |
| ATOM | 1334 | N | ASP | A | 177 | 68.925 | 54.469 | 70.148 | 1.00 | 102.03 | N |
| ATOM | 1335 | CA | ASP | A | 177 | 69.676 | 53.329 | 69.639 | 1.00 | 106.69 | C |
| ATOM | 1336 | CB | ASP | A | 177 | 69.366 | 53.153 | 68.148 | 1.00 | 107.10 | C |
| ATOM | 1337 | CG | ASP | A | 177 | 69.224 | 54.482 | 67.414 | 1.00 | 108.56 | C |
| ATOM | 1338 | OD1 | ASP | A | 177 | 68.629 | 54.492 | 66.312 | 1.00 | 108.42 | O |
| ATOM | 1339 | OD2 | ASP | A | 177 | 69.704 | 55.511 | 67.933 | 1.00 | 106.80 | O |
| ATOM | 1340 | C | ASP | A | 177 | 71.177 | 53.506 | 69.850 | 1.00 | 109.82 | C |
| ATOM | 1341 | O | ASP | A | 177 | 71.889 | 52.558 | 70.180 | 1.00 | 110.07 | O |
| ATOM | 1342 | N | GLY | A | 178 | 71.646 | 54.733 | 69.660 | 1.00 | 113.38 | N |
| ATOM | 1343 | CA | GLY | A | 178 | 73.055 | 55.029 | 69.822 | 1.00 | 117.97 | C |
| ATOM | 1344 | C | GLY | A | 178 | 73.539 | 56.001 | 68.760 | 1.00 | 121.63 | C |
| ATOM | 1345 | O | GLY | A | 178 | 73.873 | 55.591 | 67.647 | 1.00 | 121.64 | O |
| ATOM | 1346 | N | ALA | A | 179 | 73.576 | 57.286 | 69.108 | 1.00 | 125.05 | N |
| ATOM | 1347 | CA | ALA | A | 179 | 74.009 | 58.347 | 68.202 | 1.00 | 128.28 | C |
| ATOM | 1348 | CB | ALA | A | 179 | 75.263 | 57.930 | 67.440 | 1.00 | 127.71 | C |
| ATOM | 1349 | C | ALA | A | 179 | 72.907 | 58.757 | 67.223 | 1.00 | 130.50 | C |
| ATOM | 1350 | O | ALA | A | 179 | 72.038 | 57.959 | 66.872 | 1.00 | 130.91 | O |
| ATOM | 1351 | N | ALA | A | 180 | 72.963 | 60.007 | 66.769 | 1.00 | 132.73 | N |
| ATOM | 1352 | CA | ALA | A | 180 | 71.962 | 60.533 | 65.847 | 1.00 | 134.48 | C |
| ATOM | 1353 | CB | ALA | A | 180 | 71.443 | 61.878 | 66.359 | 1.00 | 133.87 | C |
| ATOM | 1354 | C | ALA | A | 180 | 72.459 | 60.672 | 64.416 | 1.00 | 135.48 | C |
| ATOM | 1355 | O | ALA | A | 180 | 72.497 | 61.774 | 63.874 | 1.00 | 136.37 | O |
| ATOM | 1356 | N | ALA | A | 181 | 72.841 | 59.555 | 63.806 | 1.00 | 136.09 | N |
| ATOM | 1357 | CA | ALA | A | 181 | 73.298 | 59.590 | 62.423 | 1.00 | 136.71 | C |
| ATOM | 1358 | CB | ALA | A | 181 | 73.699 | 58.188 | 61.960 | 1.00 | 136.50 | C |
| ATOM | 1359 | C | ALA | A | 181 | 72.136 | 60.122 | 61.586 | 1.00 | 137.08 | C |
| ATOM | 1360 | O | ALA | A | 181 | 72.176 | 61.250 | 61.101 | 1.00 | 137.20 | O |
| ATOM | 1361 | N | ALA | A | 182 | 71.099 | 59.299 | 61.449 | 1.00 | 136.96 | N |
| ATOM | 1362 | CA | ALA | A | 182 | 69.900 | 59.646 | 60.693 | 1.00 | 136.40 | C |
| ATOM | 1363 | CB | ALA | A | 182 | 68.744 | 58.724 | 61.098 | 1.00 | 136.54 | C |
| ATOM | 1364 | C | ALA | A | 182 | 69.493 | 61.108 | 60.871 | 1.00 | 135.90 | C |
| ATOM | 1365 | O | ALA | A | 182 | 69.180 | 61.790 | 59.899 | 1.00 | 135.77 | O |
| ATOM | 1366 | N | ALA | A | 183 | 69.499 | 61.574 | 62.118 | 1.00 | 134.69 | N |
| ATOM | 1367 | CA | ALA | A | 183 | 69.145 | 62.955 | 62.457 | 1.00 | 133.47 | C |
| ATOM | 1368 | CB | ALA | A | 183 | 70.393 | 63.827 | 62.437 | 1.00 | 133.43 | C |
| ATOM | 1369 | C | ALA | A | 183 | 68.086 | 63.525 | 61.511 | 1.00 | 132.35 | C |
| ATOM | 1370 | O | ALA | A | 183 | 68.409 | 64.002 | 60.424 | 1.00 | 133.00 | O |
| ATOM | 1371 | N | ALA | A | 184 | 66.832 | 63.482 | 61.957 | 1.00 | 129.94 | N |
| ATOM | 1372 | CA | ALA | A | 184 | 65.665 | 63.931 | 61.199 | 1.00 | 126.77 | C |
| ATOM | 1373 | CB | ALA | A | 184 | 66.050 | 64.940 | 60.108 | 1.00 | 126.36 | C |
| ATOM | 1374 | C | ALA | A | 184 | 65.109 | 62.660 | 60.579 | 1.00 | 124.04 | C |
| ATOM | 1375 | O | ALA | A | 184 | 65.673 | 62.114 | 59.634 | 1.00 | 124.13 | O |
| ATOM | 1376 | N | PRO | A | 185 | 64.013 | 62.141 | 61.140 | 1.00 | 121.04 | N |
| ATOM | 1377 | CD | PRO | A | 185 | 63.631 | 62.238 | 62.559 | 1.00 | 120.40 | C |
| ATOM | 1378 | CA | PRO | A | 185 | 63.470 | 60.919 | 60.558 | 1.00 | 116.89 | C |
| ATOM | 1379 | CB | PRO | A | 185 | 63.692 | 59.915 | 61.673 | 1.00 | 117.46 | C |
| ATOM | 1380 | CG | PRO | A | 185 | 63.419 | 60.769 | 62.946 | 1.00 | 119.42 | C |
| ATOM | 1381 | C | PRO | A | 185 | 61.999 | 61.018 | 60.172 | 1.00 | 112.78 | C |
| ATOM | 1382 | O | PRO | A | 185 | 61.310 | 61.981 | 60.511 | 1.00 | 113.65 | O |
| ATOM | 1383 | N | LYS | A | 186 | 61.539 | 60.011 | 59.444 | 1.00 | 106.79 | N |
| ATOM | 1384 | CA | LYS | A | 186 | 60.155 | 59.944 | 59.055 | 1.00 | 100.57 | C |
| ATOM | 1385 | CB | LYS | A | 186 | 59.952 | 58.873 | 57.987 | 1.00 | 101.15 | C |
| ATOM | 1386 | CG | LYS | A | 186 | 60.045 | 59.385 | 56.560 | 1.00 | 101.61 | C |
| ATOM | 1387 | CD | LYS | A | 186 | 61.294 | 60.221 | 56.339 | 1.00 | 100.57 | C |
| ATOM | 1388 | CE | LYS | A | 186 | 61.003 | 61.358 | 55.369 | 1.00 | 99.40 | C |
| ATOM | 1389 | NZ | LYS | A | 186 | 59.916 | 62.244 | 55.885 | 1.00 | 98.02 | N |
| ATOM | 1390 | C | LYS | A | 186 | 59.446 | 59.525 | 60.327 | 1.00 | 96.00 | C |
| ATOM | 1391 | O | LYS | A | 186 | 58.412 | 60.085 | 60.701 | 1.00 | 97.36 | O |
| ATOM | 1392 | N | HIS | A | 187 | 60.042 | 58.550 | 61.007 | 1.00 | 89.76 | N |
| ATOM | 1393 | CA | HIS | A | 187 | 59.487 | 57.989 | 62.234 | 1.00 | 84.12 | C |
| ATOM | 1394 | CB | HIS | A | 187 | 59.592 | 56.466 | 62.192 | 1.00 | 83.18 | C |
| ATOM | 1395 | CG | HIS | A | 187 | 58.861 | 55.842 | 61.050 | 1.00 | 82.83 | C |
| ATOM | 1396 | CD2 | HIS | A | 187 | 57.983 | 54.806 | 61.015 | 1.00 | 81.68 | C |
| ATOM | 1397 | ND1 | HIS | A | 187 | 59.013 | 56.253 | 59.746 | 1.00 | 82.34 | N |
| ATOM | 1398 | CE1 | HIS | A | 187 | 58.265 | 55.503 | 58.954 | 1.00 | 81.26 | C |
| ATOM | 1399 | NE2 | HIS | A | 187 | 57.632 | 54.617 | 59.706 | 1.00 | 81.76 | N |
| ATOM | 1400 | C | HIS | A | 187 | 60.160 | 58.465 | 63.505 | 1.00 | 79.36 | C |
| ATOM | 1401 | O | HIS | A | 187 | 61.353 | 58.770 | 63.519 | 1.00 | 81.01 | O |
| ATOM | 1402 | N | ILE | A | 188 | 59.379 | 58.498 | 64.575 | 1.00 | 72.24 | N |
| ATOM | 1403 | CA | ILE | A | 188 | 59.883 | 58.880 | 65.871 | 1.00 | 67.50 | C |
| ATOM | 1404 | CB | ILE | A | 188 | 59.178 | 60.128 | 66.420 | 1.00 | 68.02 | C |
| ATOM | 1405 | CG2 | ILE | A | 188 | 59.495 | 60.322 | 67.898 | 1.00 | 62.74 | C |
| ATOM | 1406 | CG1 | ILE | A | 188 | 59.639 | 61.338 | 65.596 | 1.00 | 69.84 | C |
| ATOM | 1407 | CD1 | ILE | A | 188 | 59.675 | 62.604 | 66.348 | 1.00 | 71.49 | C |
| ATOM | 1408 | C | ILE | A | 188 | 59.640 | 57.676 | 66.749 | 1.00 | 66.34 | C |
| ATOM | 1409 | O | ILE | A | 188 | 58.674 | 57.610 | 67.524 | 1.00 | 63.17 | O |
| ATOM | 1410 | N | GLY | A | 189 | 60.542 | 56.709 | 66.606 | 1.00 | 65.41 | N |
| ATOM | 1411 | CA | GLY | A | 189 | 60.439 | 55.477 | 67.362 | 1.00 | 63.73 | C |
| ATOM | 1412 | C | GLY | A | 189 | 59.429 | 54.591 | 66.669 | 1.00 | 61.42 | C |
| ATOM | 1413 | O | GLY | A | 189 | 59.587 | 54.231 | 65.508 | 1.00 | 59.32 | O |
| ATOM | 1414 | N | SER | A | 190 | 58.373 | 54.245 | 67.389 | 1.00 | 59.09 | N |
| ATOM | 1415 | CA | SER | A | 190 | 57.317 | 53.401 | 66.836 | 1.00 | 59.01 | C |
| ATOM | 1416 | CB | SER | A | 190 | 56.725 | 52.508 | 67.932 | 1.00 | 56.79 | C |
| ATOM | 1417 | OG | SER | A | 190 | 55.919 | 53.259 | 68.816 | 1.00 | 60.74 | O |
| ATOM | 1418 | C | SER | A | 190 | 56.193 | 54.240 | 66.199 | 1.00 | 57.39 | C |
| ATOM | 1419 | O | SER | A | 190 | 55.304 | 53.703 | 65.557 | 1.00 | 53.10 | O |
| ATOM | 1420 | N | ILE | A | 191 | 56.249 | 55.557 | 66.381 | 1.00 | 55.73 | N |
| ATOM | 1421 | CA | ILE | A | 191 | 55.244 | 56.471 | 65.844 | 1.00 | 55.71 | C |
| ATOM | 1422 | CB | ILE | A | 191 | 55.042 | 57.691 | 66.785 | 1.00 | 55.38 | C |
| ATOM | 1423 | CG2 | ILE | A | 191 | 54.006 | 58.651 | 66.189 | 1.00 | 54.88 | C |
| ATOM | 1424 | CG1 | ILE | A | 191 | 54.619 | 57.211 | 68.182 | 1.00 | 54.20 | C |
| ATOM | 1425 | CD1 | ILE | A | 191 | 54.391 | 58.324 | 69.169 | 1.00 | 57.91 | C |
| ATOM | 1426 | C | ILE | A | 191 | 55.574 | 57.006 | 64.451 | 1.00 | 55.25 | C |
| ATOM | 1427 | O | ILE | A | 191 | 56.722 | 57.347 | 64.156 | 1.00 | 56.70 | O |
| ATOM | 1428 | N | ASP | A | 192 | 54.559 | 57.066 | 63.592 | 1.00 | 52.62 | N |
| ATOM | 1429 | CA | ASP | A | 192 | 54.710 | 57.608 | 62.232 | 1.00 | 50.45 | C |
| ATOM | 1430 | CB | ASP | A | 192 | 54.277 | 56.590 | 61.171 | 1.00 | 49.03 | C |
| ATOM | 1431 | CG | ASP | A | 192 | 54.576 | 57.045 | 59.750 | 1.00 | 49.41 | C |
| ATOM | 1432 | OD1 | ASP | A | 192 | 54.757 | 58.252 | 59.527 | 1.00 | 51.78 | O |
| ATOM | 1433 | OD2 | ASP | A | 192 | 54.610 | 56.186 | 58.847 | 1.00 | 51.49 | O |
| ATOM | 1434 | C | ASP | A | 192 | 53.803 | 58.838 | 62.147 | 1.00 | 52.63 | C |
| ATOM | 1435 | O | ASP | A | 192 | 52.572 | 58.717 | 62.147 | 1.00 | 48.47 | O |
| ATOM | 1436 | N | PRO | A | 193 | 54.401 | 60.038 | 62.105 | 1.00 | 54.18 | N |
| ATOM | 1437 | CD | PRO | A | 193 | 55.848 | 60.290 | 62.092 | 1.00 | 56.36 | C |
| ATOM | 1438 | CA | PRO | A | 193 | 53.653 | 61.293 | 62.025 | 1.00 | 57.62 | C |
| ATOM | 1439 | CB | PRO | A | 193 | 54.737 | 62.349 | 62.245 | 1.00 | 58.21 | C |
| ATOM | 1440 | CG | PRO | A | 193 | 55.913 | 61.723 | 61.603 | 1.00 | 59.28 | C |
| ATOM | 1441 | C | PRO | A | 193 | 52.914 | 61.442 | 60.696 | 1.00 | 55.86 | C |
| ATOM | 1442 | O | PRO | A | 193 | 52.099 | 62.341 | 60.540 | 1.00 | 55.22 | O |
| ATOM | 1443 | N | ASN | A | 194 | 53.207 | 60.552 | 59.749 | 1.00 | 55.86 | N |
| ATOM | 1444 | CA | ASN | A | 194 | 52.551 | 60.557 | 58.441 | 1.00 | 57.86 | C |
| ATOM | 1445 | CB | ASN | A | 194 | 53.437 | 61.189 | 57.377 | 1.00 | 62.71 | C |
| ATOM | 1446 | CG | ASN | A | 194 | 53.223 | 62.680 | 57.273 | 1.00 | 70.78 | C |
| ATOM | 1447 | OD1 | ASN | A | 194 | 53.743 | 63.460 | 58.086 | 1.00 | 69.84 | O |
| ATOM | 1448 | ND2 | ASN | A | 194 | 52.431 | 63.091 | 56.281 | 1.00 | 73.33 | N |
| ATOM | 1449 | C | ASN | A | 194 | 52.139 | 59.180 | 57.969 | 1.00 | 55.99 | C |
| ATOM | 1450 | O | ASN | A | 194 | 52.427 | 58.793 | 56.852 | 1.00 | 54.80 | O |
| ATOM | 1451 | N | CYS | A | 195 | 51.455 | 58.446 | 58.831 | 1.00 | 52.67 | N |
| ATOM | 1452 | CA | CYS | A | 195 | 51.012 | 57.106 | 58.512 | 1.00 | 50.74 | C |
| ATOM | 1453 | CB | CYS | A | 195 | 50.557 | 56.416 | 59.803 | 1.00 | 50.72 | C |
| ATOM | 1454 | SG | CYS | A | 195 | 49.915 | 54.754 | 59.560 | 1.00 | 47.33 | S |
| ATOM | 1455 | C | CYS | A | 195 | 49.900 | 57.030 | 57.450 | 1.00 | 51.45 | C |
| ATOM | 1456 | O | CYS | A | 195 | 48.838 | 57.646 | 57.597 | 1.00 | 47.57 | O |
| ATOM | 1457 | N | ASN | A | 196 | 50.161 | 56.283 | 56.376 | 1.00 | 51.03 | N |
| ATOM | 1458 | CA | ASN | A | 196 | 49.175 | 56.091 | 55.314 | 1.00 | 51.22 | C |
| ATOM | 1459 | CB | ASN | A | 196 | 49.872 | 55.949 | 53.954 | 1.00 | 51.67 | C |
| ATOM | 1460 | CG | ASN | A | 196 | 48.893 | 55.719 | 52.813 | 1.00 | 55.47 | C |
| ATOM | 1461 | OD1 | ASN | A | 196 | 47.682 | 55.863 | 52.970 | 1.00 | 61.54 | O |
| ATOM | 1462 | ND2 | ASN | A | 196 | 49.424 | 55.376 | 51.651 | 1.00 | 58.85 | N |
| ATOM | 1463 | C | ASN | A | 196 | 48.366 | 54.825 | 55.655 | 1.00 | 51.12 | C |
| ATOM | 1464 | O | ASN | A | 196 | 48.808 | 53.696 | 55.398 | 1.00 | 49.62 | O |
| ATOM | 1465 | N | VAL | A | 197 | 47.178 | 55.041 | 56.224 | 1.00 | 48.52 | N |
| ATOM | 1466 | CA | VAL | A | 197 | 46.300 | 53.964 | 56.648 | 1.00 | 45.39 | C |
| ATOM | 1467 | CB | VAL | A | 197 | 44.931 | 54.522 | 57.095 | 1.00 | 42.62 | C |
| ATOM | 1468 | CG1 | VAL | A | 197 | 43.993 | 53.387 | 57.528 | 1.00 | 40.48 | C |
| ATOM | 1469 | CG2 | VAL | A | 197 | 45.133 | 55.496 | 58.237 | 1.00 | 41.12 | C |
| ATOM | 1470 | C | VAL | A | 197 | 46.088 | 52.858 | 55.615 | 1.00 | 47.54 | C |
| ATOM | 1471 | O | VAL | A | 197 | 46.245 | 51.664 | 55.926 | 1.00 | 47.26 | O |
| ATOM | 1472 | N | SER | A | 198 | 45.738 | 53.244 | 54.391 | 1.00 | 48.17 | N |
| ATOM | 1473 | CA | SER | A | 198 | 45.502 | 52.261 | 53.345 | 1.00 | 51.18 | C |
| ATOM | 1474 | CB | SER | A | 198 | 45.148 | 52.954 | 52.033 | 1.00 | 51.44 | C |
| ATOM | 1475 | OG | SER | A | 198 | 43.774 | 53.259 | 51.977 | 1.00 | 57.13 | O |
| ATOM | 1476 | C | SER | A | 198 | 46.701 | 51.337 | 53.128 | 1.00 | 51.50 | C |
| ATOM | 1477 | O | SER | A | 198 | 46.529 | 50.138 | 52.870 | 1.00 | 50.27 | O |
| ATOM | 1478 | N | GLU | A | 199 | 47.909 | 51.887 | 53.234 | 1.00 | 53.20 | N |
| ATOM | 1479 | CA | GLU | A | 199 | 49.107 | 51.081 | 53.038 | 1.00 | 53.64 | C |
| ATOM | 1480 | CB | GLU | A | 199 | 50.368 | 51.954 | 53.080 | 1.00 | 58.68 | C |
| ATOM | 1481 | CG | GLU | A | 199 | 51.627 | 51.246 | 52.584 | 1.00 | 66.73 | C |
| ATOM | 1482 | CD | GLU | A | 199 | 51.405 | 50.487 | 51.268 | 1.00 | 72.78 | C |
| ATOM | 1483 | OE1 | GLU | A | 199 | 50.791 | 51.067 | 50.337 | 1.00 | 76.61 | O |
| ATOM | 1484 | OE2 | GLU | A | 199 | 51.852 | 49.318 | 51.168 | 1.00 | 73.70 | O |
| ATOM | 1485 | C | GLU | A | 199 | 49.212 | 49.975 | 54.086 | 1.00 | 51.04 | C |
| ATOM | 1486 | O | GLU | A | 199 | 49.558 | 48.825 | 53.775 | 1.00 | 46.37 | O |
| ATOM | 1487 | N | VAL | A | 200 | 48.919 | 50.327 | 55.333 | 1.00 | 46.89 | N |
| ATOM | 1488 | CA | VAL | A | 200 | 48.976 | 49.360 | 56.407 | 1.00 | 45.34 | C |
| ATOM | 1489 | CB | VAL | A | 200 | 48.800 | 50.038 | 57.757 | 1.00 | 43.69 | C |
| ATOM | 1490 | CG1 | VAL | A | 200 | 49.006 | 49.034 | 58.888 | 1.00 | 38.15 | C |
| ATOM | 1491 | CG2 | VAL | A | 200 | 49.784 | 51.187 | 57.874 | 1.00 | 42.28 | C |
| ATOM | 1492 | C | VAL | A | 200 | 47.893 | 48.302 | 56.203 | 1.00 | 45.99 | C |
| ATOM | 1493 | O | VAL | A | 200 | 48.126 | 47.113 | 56.479 | 1.00 | 45.31 | O |
| ATOM | 1494 | N | VAL | A | 201 | 46.730 | 48.728 | 55.691 | 1.00 | 42.26 | N |
| ATOM | 1495 | CA | VAL | A | 201 | 45.619 | 47.808 | 55.421 | 1.00 | 43.03 | C |
| ATOM | 1496 | CB | VAL | A | 201 | 44.386 | 48.546 | 54.764 | 1.00 | 42.08 | C |
| ATOM | 1497 | CG1 | VAL | A | 201 | 43.331 | 47.530 | 54.350 | 1.00 | 37.05 | C |
| ATOM | 1498 | CG2 | VAL | A | 201 | 43.764 | 49.552 | 55.736 | 1.00 | 38.14 | C |
| ATOM | 1499 | C | VAL | A | 201 | 46.140 | 46.751 | 54.440 | 1.00 | 41.69 | C |
| ATOM | 1500 | O | VAL | A | 201 | 46.047 | 45.537 | 54.662 | 1.00 | 34.17 | O |
| ATOM | 1501 | N | LYS | A | 202 | 46.698 | 47.257 | 53.347 | 1.00 | 45.79 | N |
| ATOM | 1502 | CA | LYS | A | 202 | 47.269 | 46.459 | 52.255 | 1.00 | 51.90 | C |
| ATOM | 1503 | CB | LYS | A | 202 | 47.919 | 47.415 | 51.237 | 1.00 | 55.82 | C |
| ATOM | 1504 | CG | LYS | A | 202 | 47.643 | 47.117 | 49.776 | 1.00 | 60.81 | C |
| ATOM | 1505 | CD | LYS | A | 202 | 46.952 | 48.312 | 49.078 | 1.00 | 66.67 | C |
| ATOM | 1506 | CE | LYS | A | 202 | 47.883 | 49.518 | 48.878 | 1.00 | 68.16 | C |
| ATOM | 1507 | NZ | LYS | A | 202 | 47.143 | 50.781 | 48.562 | 1.00 | 67.08 | N |
| ATOM | 1508 | C | LYS | A | 202 | 48.301 | 45.453 | 52.777 | 1.00 | 51.83 | C |
| ATOM | 1509 | O | LYS | A | 202 | 48.265 | 44.291 | 52.402 | 1.00 | 51.27 | O |
| ATOM | 1510 | N | ASP | A | 203 | 49.186 | 45.905 | 53.666 | 1.00 | 47.78 | N |
| ATOM | 1511 | CA | ASP | A | 203 | 50.218 | 45.041 | 54.218 | 1.00 | 49.39 | C |
| ATOM | 1512 | CB | ASP | A | 203 | 51.182 | 45.827 | 55.109 | 1.00 | 52.58 | C |
| ATOM | 1513 | CG | ASP | A | 203 | 52.065 | 46.776 | 54.320 | 1.00 | 58.11 | C |
| ATOM | 1514 | OD1 | ASP | A | 203 | 52.265 | 46.537 | 53.111 | 1.00 | 62.03 | O |
| ATOM | 1515 | OD2 | ASP | A | 203 | 52.566 | 47.753 | 54.908 | 1.00 | 57.73 | O |
| ATOM | 1516 | C | ASP | A | 203 | 49.644 | 43.885 | 55.018 | 1.00 | 50.35 | C |
| ATOM | 1517 | O | ASP | A | 203 | 49.989 | 42.715 | 54.785 | 1.00 | 47.82 | O |
| ATOM | 1518 | N | ALA | A | 204 | 48.785 | 44.218 | 55.981 | 1.00 | 49.02 | N |
| ATOM | 1519 | CA | ALA | A | 204 | 48.144 | 43.209 | 56.823 | 1.00 | 45.68 | C |
| ATOM | 1520 | CB | ALA | A | 204 | 47.221 | 43.864 | 57.845 | 1.00 | 43.54 | C |
| ATOM | 1521 | C | ALA | A | 204 | 47.352 | 42.243 | 55.947 | 1.00 | 44.58 | C |
| ATOM | 1522 | O | ALA | A | 204 | 47.338 | 41.022 | 56.198 | 1.00 | 38.04 | O |
| ATOM | 1523 | N | TYR | A | 205 | 46.693 | 42.775 | 54.916 | 1.00 | 45.59 | N |
| ATOM | 1524 | CA | TYR | A | 205 | 45.936 | 41.905 | 54.014 | 1.00 | 50.52 | C |
| ATOM | 1525 | CB | TYR | A | 205 | 45.254 | 42.724 | 52.929 | 1.00 | 50.59 | C |
| ATOM | 1526 | CG | TYR | A | 205 | 44.720 | 41.865 | 51.826 | 1.00 | 55.83 | C |
| ATOM | 1527 | CD1 | TYR | A | 205 | 43.527 | 41.150 | 51.982 | 1.00 | 57.93 | C |
| ATOM | 1528 | CE1 | TYR | A | 205 | 43.049 | 40.300 | 50.964 | 1.00 | 58.71 | C |
| ATOM | 1529 | CD2 | TYR | A | 205 | 45.431 | 41.721 | 50.635 | 1.00 | 55.17 | C |
| ATOM | 1530 | CE2 | TYR | A | 205 | 44.976 | 40.878 | 49.618 | 1.00 | 58.27 | C |
| ATOM | 1531 | CZ | TYR | A | 205 | 43.787 | 40.173 | 49.790 | 1.00 | 61.37 | C |
| ATOM | 1532 | OH | TYR | A | 205 | 43.357 | 39.339 | 48.793 | 1.00 | 57.60 | O |
| ATOM | 1533 | C | TYR | A | 205 | 46.855 | 40.860 | 53.362 | 1.00 | 49.90 | C |
| ATOM | 1534 | O | TYR | A | 205 | 46.601 | 39.664 | 53.443 | 1.00 | 47.11 | O |
| ATOM | 1535 | N | ASP | A | 206 | 47.933 | 41.334 | 52.737 | 1.00 | 48.89 | N |
| ATOM | 1536 | CA | ASP | A | 206 | 48.906 | 40.469 | 52.064 | 1.00 | 51.87 | C |
| ATOM | 1537 | CB | ASP | A | 206 | 50.055 | 41.307 | 51.462 | 1.00 | 51.86 | C |
| ATOM | 1538 | CG | ASP | A | 206 | 49.610 | 42.163 | 50.281 | 1.00 | 56.20 | C |
| ATOM | 1539 | OD1 | ASP | A | 206 | 48.780 | 41.701 | 49.473 | 1.00 | 57.68 | O |
| ATOM | 1540 | OD2 | ASP | A | 206 | 50.104 | 43.298 | 50.152 | 1.00 | 55.76 | O |
| ATOM | 1541 | C | ASP | A | 206 | 49.483 | 39.374 | 52.969 | 1.00 | 50.35 | C |
| ATOM | 1542 | O | ASP | A | 206 | 49.680 | 38.241 | 52.539 | 1.00 | 47.14 | O |
| ATOM | 1543 | N | MET | A | 207 | 49.754 | 39.714 | 54.222 | 1.00 | 49.01 | N |
| ATOM | 1544 | CA | MET | A | 207 | 50.297 | 38.737 | 55.147 | 1.00 | 49.15 | C |
| ATOM | 1545 | CB | MET | A | 207 | 50.753 | 39.407 | 56.433 | 1.00 | 48.30 | C |
| ATOM | 1546 | CG | MET | A | 207 | 51.505 | 38.456 | 57.369 | 1.00 | 57.29 | C |
| ATOM | 1547 | SD | MET | A | 207 | 52.993 | 37.679 | 56.564 | 1.00 | 58.09 | S |
| ATOM | 1548 | CE | MET | A | 207 | 52.618 | 35.913 | 56.863 | 1.00 | 54.59 | C |
| ATOM | 1549 | C | MET | A | 207 | 49.259 | 37.681 | 55.489 | 1.00 | 51.04 | C |
| ATOM | 1550 | O | MET | A | 207 | 49.596 | 36.493 | 55.631 | 1.00 | 46.06 | O |
| ATOM | 1551 | N | ALA | A | 208 | 48.006 | 38.122 | 55.635 | 1.00 | 51.34 | N |
| ATOM | 1552 | CA | ALA | A | 208 | 46.918 | 37.219 | 55.974 | 1.00 | 50.25 | C |
| ATOM | 1553 | CB | ALA | A | 208 | 45.669 | 38.013 | 56.331 | 1.00 | 48.81 | C |
| ATOM | 1554 | C | ALA | A | 208 | 46.648 | 36.286 | 54.794 | 1.00 | 50.40 | C |
| ATOM | 1555 | O | ALA | A | 208 | 46.466 | 35.090 | 54.976 | 1.00 | 45.65 | O |
| ATOM | 1556 | N | LYS | A | 209 | 46.650 | 36.855 | 53.590 | 1.00 | 51.41 | N |
| ATOM | 1557 | CA | LYS | A | 209 | 46.408 | 36.129 | 52.345 | 1.00 | 58.65 | C |
| ATOM | 1558 | CB | LYS | A | 209 | 46.544 | 37.063 | 51.147 | 1.00 | 58.64 | C |
| ATOM | 1559 | CG | LYS | A | 209 | 46.578 | 36.326 | 49.813 | 1.00 | 60.86 | C |
| ATOM | 1560 | CD | LYS | A | 209 | 46.563 | 37.314 | 48.656 | 1.00 | 63.37 | C |
| ATOM | 1561 | CE | LYS | A | 209 | 46.278 | 36.622 | 47.327 | 1.00 | 66.28 | C |
| ATOM | 1562 | NZ | LYS | A | 209 | 45.831 | 37.593 | 46.325 | 1.00 | 70.25 | N |
| ATOM | 1563 | C | LYS | A | 209 | 47.391 | 35.001 | 52.165 | 1.00 | 62.38 | C |
| ATOM | 1564 | O | LYS | A | 209 | 47.039 | 33.901 | 51.740 | 1.00 | 59.81 | O |
| ATOM | 1565 | N | LEU | A | 210 | 48.644 | 35.307 | 52.465 | 1.00 | 65.63 | N |
| ATOM | 1566 | CA | LEU | A | 210 | 49.704 | 34.328 | 52.362 | 1.00 | 67.90 | C |
| ATOM | 1567 | CB | LEU | A | 210 | 51.015 | 34.967 | 52.821 | 1.00 | 71.92 | C |
| ATOM | 1568 | CG | LEU | A | 210 | 52.324 | 34.325 | 52.374 | 1.00 | 73.64 | C |
| ATOM | 1569 | CD1 | LEU | A | 210 | 53.481 | 35.280 | 52.621 | 1.00 | 74.00 | C |
| ATOM | 1570 | CD2 | LEU | A | 210 | 52.524 | 33.021 | 53.108 | 1.00 | 75.58 | C |
| ATOM | 1571 | C | LEU | A | 210 | 49.305 | 33.136 | 53.239 | 1.00 | 65.07 | C |
| ATOM | 1572 | O | LEU | A | 210 | 49.165 | 32.024 | 52.741 | 1.00 | 66.82 | O |
| ATOM | 1573 | N | LEU | A | 211 | 49.087 | 33.374 | 54.528 | 1.00 | 61.66 | N |
| ATOM | 1574 | CA | LEU | A | 211 | 48.676 | 32.307 | 55.419 | 1.00 | 61.79 | C |
| ATOM | 1575 | CB | LEU | A | 211 | 48.404 | 32.849 | 56.820 | 1.00 | 59.29 | C |
| ATOM | 1576 | CG | LEU | A | 211 | 49.604 | 33.176 | 57.707 | 1.00 | 58.42 | C |
| ATOM | 1577 | CD1 | LEU | A | 211 | 49.149 | 33.977 | 58.911 | 1.00 | 55.47 | C |
| ATOM | 1578 | CD2 | LEU | A | 211 | 50.280 | 31.893 | 58.153 | 1.00 | 61.59 | C |
| ATOM | 1579 | C | LEU | A | 211 | 47.426 | 31.602 | 54.901 | 1.00 | 65.38 | C |
| ATOM | 1580 | O | LEU | A | 211 | 47.374 | 30.360 | 54.873 | 1.00 | 63.72 | O |
| ATOM | 1581 | N | CYS | A | 212 | 46.421 | 32.377 | 54.496 | 1.00 | 68.69 | N |
| ATOM | 1582 | CA | CYS | A | 212 | 45.192 | 31.772 | 54.005 | 1.00 | 71.81 | C |
| ATOM | 1583 | CB | CYS | A | 212 | 44.186 | 32.857 | 53.624 | 1.00 | 71.77 | C |
| ATOM | 1584 | SG | CYS | A | 212 | 42.521 | 32.249 | 53.213 | 1.00 | 74.51 | S |
| ATOM | 1585 | C | CYS | A | 212 | 45.426 | 30.816 | 52.819 | 1.00 | 70.79 | C |
| ATOM | 1586 | O | CYS | A | 212 | 44.881 | 29.710 | 52.791 | 1.00 | 66.24 | O |
| ATOM | 1587 | N | ASP | A | 213 | 46.247 | 31.232 | 51.858 | 1.00 | 70.90 | N |
| ATOM | 1588 | CA | ASP | A | 213 | 46.548 | 30.406 | 50.687 | 1.00 | 72.95 | C |
| ATOM | 1589 | CB | ASP | A | 213 | 47.403 | 31.181 | 49.689 | 1.00 | 68.60 | C |
| ATOM | 1590 | CG | ASP | A | 213 | 46.602 | 32.174 | 48.913 | 1.00 | 68.77 | C |
| ATOM | 1591 | OD1 | ASP | A | 213 | 45.378 | 31.961 | 48.830 | 1.00 | 67.14 | O |
| ATOM | 1592 | OD2 | ASP | A | 213 | 47.180 | 33.149 | 48.389 | 1.00 | 65.80 | O |
| ATOM | 1593 | C | ASP | A | 213 | 47.263 | 29.112 | 51.021 | 1.00 | 76.22 | C |
| ATOM | 1594 | O | ASP | A | 213 | 46.986 | 28.059 | 50.447 | 1.00 | 77.62 | O |
| ATOM | 1595 | N | LYS | A | 214 | 48.203 | 29.196 | 51.947 | 1.00 | 79.26 | N |
| ATOM | 1596 | CA | LYS | A | 214 | 48.965 | 28.025 | 52.350 | 1.00 | 82.90 | C |
| ATOM | 1597 | CB | LYS | A | 214 | 50.092 | 28.456 | 53.290 | 1.00 | 84.59 | C |
| ATOM | 1598 | CG | LYS | A | 214 | 50.988 | 27.328 | 53.739 | 1.00 | 87.42 | C |
| ATOM | 1599 | CD | LYS | A | 214 | 51.775 | 27.722 | 54.981 | 1.00 | 90.13 | C |
| ATOM | 1600 | CE | LYS | A | 214 | 52.552 | 26.528 | 55.533 | 1.00 | 91.42 | C |
| ATOM | 1601 | NZ | LYS | A | 214 | 53.090 | 26.769 | 56.899 | 1.00 | 90.95 | N |
| ATOM | 1602 | C | LYS | A | 214 | 48.083 | 26.974 | 53.034 | 1.00 | 84.03 | C |
| ATOM | 1603 | O | LYS | A | 214 | 48.231 | 25.786 | 52.794 | 1.00 | 84.53 | O |
| ATOM | 1604 | N | TYR | A | 215 | 47.154 | 27.423 | 53.869 | 1.00 | 85.90 | N |
| ATOM | 1605 | CA | TYR | A | 215 | 46.274 | 26.517 | 54.602 | 1.00 | 87.30 | C |
| ATOM | 1606 | CB | TYR | A | 215 | 45.903 | 27.145 | 55.948 | 1.00 | 91.94 | C |
| ATOM | 1607 | CG | TYR | A | 215 | 45.271 | 26.192 | 56.947 | 1.00 | 97.53 | C |
| ATOM | 1608 | CD1 | TYR | A | 215 | 46.017 | 25.171 | 57.530 | 1.00 | 99.66 | C |
| ATOM | 1609 | CE1 | TYR | A | 215 | 45.446 | 24.309 | 58.476 | 1.00 | 101.78 | C |
| ATOM | 1610 | CD2 | TYR | A | 215 | 43.934 | 26.329 | 57.328 | 1.00 | 99.14 | C |
| ATOM | 1611 | CE2 | TYR | A | 215 | 43.354 | 25.480 | 58.264 | 1.00 | 100.48 | C |
| ATOM | 1612 | CZ | TYR | A | 215 | 44.112 | 24.472 | 58.836 | 1.00 | 101.96 | C |
| ATOM | 1613 | OH | TYR | A | 215 | 43.543 | 23.631 | 59.771 | 1.00 | 100.47 | O |
| ATOM | 1614 | C | TYR | A | 215 | 44.996 | 26.099 | 53.876 | 1.00 | 86.44 | C |
| ATOM | 1615 | O | TYR | A | 215 | 44.545 | 24.971 | 54.025 | 1.00 | 86.18 | O |
| ATOM | 1616 | N | TYR | A | 216 | 44.406 | 27.001 | 53.102 | 1.00 | 85.75 | N |
| ATOM | 1617 | CA | TYR | A | 216 | 43.173 | 26.685 | 52.388 | 1.00 | 85.10 | C |
| ATOM | 1618 | CB | TYR | A | 216 | 42.068 | 27.686 | 52.752 | 1.00 | 85.85 | C |
| ATOM | 1619 | CG | TYR | A | 216 | 41.684 | 27.728 | 54.220 | 1.00 | 85.10 | C |
| ATOM | 1620 | CD1 | TYR | A | 216 | 42.184 | 28.720 | 55.070 | 1.00 | 84.40 | C |
| ATOM | 1621 | CE1 | TYR | A | 216 | 41.824 | 28.767 | 56.412 | 1.00 | 85.14 | C |
| ATOM | 1622 | CD2 | TYR | A | 216 | 40.812 | 26.784 | 54.754 | 1.00 | 85.19 | C |
| ATOM | 1623 | CE2 | TYR | A | 216 | 40.444 | 26.818 | 56.096 | 1.00 | 85.32 | C |
| ATOM | 1624 | CZ | TYR | A | 216 | 40.948 | 27.810 | 56.922 | 1.00 | 86.24 | C |
| ATOM | 1625 | OH | TYR | A | 216 | 40.581 | 27.839 | 58.255 | 1.00 | 85.29 | O |
| ATOM | 1626 | C | TYR | A | 216 | 43.340 | 26.676 | 50.877 | 1.00 | 85.26 | C |
| ATOM | 1627 | O | TYR | A | 216 | 42.428 | 26.292 | 50.149 | 1.00 | 84.52 | O |
| ATOM | 1628 | N | MET | A | 217 | 44.498 | 27.120 | 50.408 | 1.00 | 87.92 | N |
| ATOM | 1629 | CA | MET | A | 217 | 44.779 | 27.159 | 48.975 | 1.00 | 90.66 | C |
| ATOM | 1630 | CB | MET | A | 217 | 45.082 | 25.746 | 48.477 | 1.00 | 96.48 | C |
| ATOM | 1631 | CG | MET | A | 217 | 46.235 | 25.119 | 49.255 | 1.00 | 103.08 | C |
| ATOM | 1632 | SD | MET | A | 217 | 46.693 | 23.473 | 48.744 | 1.00 | 111.96 | S |
| ATOM | 1633 | CE | MET | A | 217 | 48.437 | 23.492 | 49.140 | 1.00 | 109.84 | C |
| ATOM | 1634 | C | MET | A | 217 | 43.634 | 27.797 | 48.191 | 1.00 | 88.20 | C |
| ATOM | 1635 | O | MET | A | 217 | 43.174 | 27.283 | 47.164 | 1.00 | 87.20 | O |
| ATOM | 1636 | N | ALA | A | 218 | 43.201 | 28.942 | 48.709 | 1.00 | 84.82 | N |
| ATOM | 1637 | CA | ALA | A | 218 | 42.130 | 29.761 | 48.149 | 1.00 | 78.42 | C |
| ATOM | 1638 | CB | ALA | A | 218 | 40.815 | 28.996 | 48.155 | 1.00 | 78.85 | C |
| ATOM | 1639 | C | ALA | A | 218 | 42.045 | 30.970 | 49.082 | 1.00 | 74.28 | C |
| ATOM | 1640 | O | ALA | A | 218 | 42.199 | 30.828 | 50.302 | 1.00 | 71.27 | O |
| ATOM | 1641 | N | SER | A | 219 | 41.832 | 32.155 | 48.526 | 1.00 | 68.36 | N |
| ATOM | 1642 | CA | SER | A | 219 | 41.735 | 33.324 | 49.371 | 1.00 | 64.11 | C |
| ATOM | 1643 | CB | SER | A | 219 | 43.129 | 33.828 | 49.800 | 1.00 | 66.11 | C |
| ATOM | 1644 | OG | SER | A | 219 | 43.758 | 34.565 | 48.772 | 1.00 | 63.25 | O |
| ATOM | 1645 | C | SER | A | 219 | 40.975 | 34.423 | 48.666 | 1.00 | 62.37 | C |
| ATOM | 1646 | O | SER | A | 219 | 41.138 | 34.642 | 47.469 | 1.00 | 59.21 | O |
| ATOM | 1647 | N | PRO | A | 220 | 40.113 | 35.123 | 49.414 | 1.00 | 61.24 | N |
| ATOM | 1648 | CD | PRO | A | 220 | 39.819 | 34.883 | 50.842 | 1.00 | 58.41 | C |
| ATOM | 1649 | CA | PRO | A | 220 | 39.306 | 36.220 | 48.877 | 1.00 | 57.82 | C |
| ATOM | 1650 | CB | PRO | A | 220 | 38.326 | 36.500 | 50.014 | 1.00 | 59.42 | C |
| ATOM | 1651 | CG | PRO | A | 220 | 39.126 | 36.146 | 51.246 | 1.00 | 60.18 | C |
| ATOM | 1652 | C | PRO | A | 220 | 40.204 | 37.404 | 48.553 | 1.00 | 55.73 | C |
| ATOM | 1653 | O | PRO | A | 220 | 41.287 | 37.520 | 49.112 | 1.00 | 52.46 | O |
| ATOM | 1654 | N | ASP | A | 221 | 39.764 | 38.272 | 47.646 | 1.00 | 54.13 | N |
| ATOM | 1655 | CA | ASP | A | 221 | 40.559 | 39.441 | 47.267 | 1.00 | 56.91 | C |
| ATOM | 1656 | CB | ASP | A | 221 | 40.357 | 39.806 | 45.784 | 1.00 | 61.70 | C |
| ATOM | 1657 | CG | ASP | A | 221 | 40.833 | 38.707 | 44.831 | 1.00 | 66.26 | C |
| ATOM | 1658 | OD1 | ASP | A | 221 | 41.852 | 38.031 | 45.128 | 1.00 | 66.17 | O |
| ATOM | 1659 | OD2 | ASP | A | 221 | 40.195 | 38.526 | 43.772 | 1.00 | 63.72 | O |
| ATOM | 1660 | C | ASP | A | 221 | 40.228 | 40.652 | 48.128 | 1.00 | 57.95 | C |
| ATOM | 1661 | O | ASP | A | 221 | 39.378 | 40.579 | 48.999 | 1.00 | 58.23 | O |
| ATOM | 1662 | N | LEU | A | 222 | 40.910 | 41.763 | 47.880 | 1.00 | 55.73 | N |
| ATOM | 1663 | CA | LEU | A | 222 | 40.694 | 42.983 | 48.635 | 1.00 | 55.70 | C |
| ATOM | 1664 | CB | LEU | A | 222 | 41.974 | 43.390 | 49.391 | 1.00 | 57.29 | C |
| ATOM | 1665 | CG | LEU | A | 222 | 41.967 | 44.779 | 50.065 | 1.00 | 56.01 | C |
| ATOM | 1666 | CD1 | LEU | A | 222 | 40.980 | 44.779 | 51.220 | 1.00 | 56.58 | C |
| ATOM | 1667 | CD2 | LEU | A | 222 | 43.359 | 45.131 | 50.545 | 1.00 | 56.83 | C |
| ATOM | 1668 | C | LEU | A | 222 | 40.255 | 44.153 | 47.758 | 1.00 | 55.70 | C |
| ATOM | 1669 | O | LEU | A | 222 | 40.686 | 44.276 | 46.605 | 1.00 | 54.26 | O |
| ATOM | 1670 | N | GLU | A | 223 | 39.399 | 44.998 | 48.329 | 1.00 | 54.43 | N |
| ATOM | 1671 | CA | GLU | A | 223 | 38.897 | 46.197 | 47.679 | 1.00 | 54.44 | C |
| ATOM | 1672 | CB | GLU | A | 223 | 37.417 | 46.052 | 47.295 | 1.00 | 57.46 | C |
| ATOM | 1673 | CG | GLU | A | 223 | 37.163 | 45.267 | 46.031 | 1.00 | 65.38 | C |
| ATOM | 1674 | CD | GLU | A | 223 | 35.686 | 45.252 | 45.615 | 1.00 | 70.64 | C |
| ATOM | 1675 | OE1 | GLU | A | 223 | 35.055 | 46.335 | 45.596 | 1.00 | 68.97 | O |
| ATOM | 1676 | OE2 | GLU | A | 223 | 35.164 | 44.157 | 45.296 | 1.00 | 73.35 | O |
| ATOM | 1677 | C | GLU | A | 223 | 39.036 | 47.285 | 48.739 | 1.00 | 53.39 | C |
| ATOM | 1678 | O | GLU | A | 223 | 38.649 | 47.061 | 49.894 | 1.00 | 53.11 | O |
| ATOM | 1679 | N | ILE | A | 224 | 39.578 | 48.444 | 48.375 | 1.00 | 49.92 | N |
| ATOM | 1680 | CA | ILE | A | 224 | 39.714 | 49.500 | 49.355 | 1.00 | 51.13 | C |
| ATOM | 1681 | CB | ILE | A | 224 | 41.202 | 49.808 | 49.693 | 1.00 | 51.44 | C |
| ATOM | 1682 | CG2 | ILE | A | 224 | 41.289 | 51.039 | 50.630 | 1.00 | 42.88 | C |
| ATOM | 1683 | CG1 | ILE | A | 224 | 41.855 | 48.590 | 50.340 | 1.00 | 47.34 | C |
| ATOM | 1684 | CD1 | ILE | A | 224 | 43.295 | 48.815 | 50.732 | 1.00 | 51.24 | C |
| ATOM | 1685 | C | ILE | A | 224 | 39.072 | 50.768 | 48.864 | 1.00 | 52.92 | C |
| ATOM | 1686 | O | ILE | A | 224 | 39.252 | 51.162 | 47.725 | 1.00 | 55.48 | O |
| ATOM | 1687 | N | GLN | A | 225 | 38.342 | 51.424 | 49.751 | 1.00 | 54.61 | N |
| ATOM | 1688 | CA | GLN | A | 225 | 37.705 | 52.675 | 49.402 | 1.00 | 55.13 | C |
| ATOM | 1689 | CB | GLN | A | 225 | 36.211 | 52.476 | 49.207 | 1.00 | 57.07 | C |
| ATOM | 1690 | CG | GLN | A | 225 | 35.548 | 53.677 | 48.550 | 1.00 | 68.45 | C |
| ATOM | 1691 | CD | GLN | A | 225 | 34.038 | 53.542 | 48.417 | 1.00 | 76.66 | C |
| ATOM | 1692 | OE1 | GLN | A | 225 | 33.513 | 52.441 | 48.227 | 1.00 | 80.86 | O |
| ATOM | 1693 | NE2 | GLN | A | 225 | 33.331 | 54.673 | 48.496 | 1.00 | 77.46 | N |
| ATOM | 1694 | C | GLN | A | 225 | 37.934 | 53.734 | 50.470 | 1.00 | 52.69 | C |
| ATOM | 1695 | O | GLN | A | 225 | 37.688 | 53.502 | 51.644 | 1.00 | 53.38 | O |
| ATOM | 1696 | N | GLU | A | 226 | 38.418 | 54.893 | 50.052 | 1.00 | 51.80 | N |
| ATOM | 1697 | CA | GLU | A | 226 | 38.657 | 55.975 | 50.974 | 1.00 | 52.23 | C |
| ATOM | 1698 | CB | GLU | A | 226 | 40.048 | 56.568 | 50.727 | 1.00 | 49.76 | C |
| ATOM | 1699 | CG | GLU | A | 226 | 41.174 | 55.693 | 51.248 | 1.00 | 55.54 | C |
| ATOM | 1700 | CD | GLU | A | 226 | 42.507 | 56.402 | 51.292 | 1.00 | 60.11 | C |
| ATOM | 1701 | OE1 | GLU | A | 226 | 42.524 | 57.646 | 51.415 | 1.00 | 57.89 | O |
| ATOM | 1702 | OE2 | GLU | A | 226 | 43.540 | 55.705 | 51.228 | 1.00 | 62.40 | O |
| ATOM | 1703 | C | GLU | A | 226 | 37.576 | 57.039 | 50.852 | 1.00 | 51.45 | C |
| ATOM | 1704 | O | GLU | A | 226 | 37.022 | 57.241 | 49.778 | 1.00 | 53.09 | O |
| ATOM | 1705 | N | ILE | A | 227 | 37.272 | 57.697 | 51.968 | 1.00 | 49.09 | N |
| ATOM | 1706 | CA | ILE | A | 227 | 36.267 | 58.762 | 52.028 | 1.00 | 49.04 | C |
| ATOM | 1707 | CB | ILE | A | 227 | 34.948 | 58.287 | 52.730 | 1.00 | 51.94 | C |
| ATOM | 1708 | CG2 | ILE | A | 227 | 33.882 | 59.375 | 52.650 | 1.00 | 46.41 | C |
| ATOM | 1709 | CG1 | ILE | A | 227 | 34.435 | 57.002 | 52.072 | 1.00 | 53.89 | C |
| ATOM | 1710 | CD1 | ILE | A | 227 | 33.981 | 57.178 | 50.641 | 1.00 | 52.12 | C |
| ATOM | 1711 | C | ILE | A | 227 | 36.835 | 59.935 | 52.825 | 1.00 | 51.34 | C |
| ATOM | 1712 | O | ILE | A | 227 | 36.669 | 60.006 | 54.040 | 1.00 | 50.71 | O |
| ATOM | 1713 | N | ASN | A | 228 | 37.510 | 60.853 | 52.151 | 1.00 | 52.82 | N |
| ATOM | 1714 | CA | ASN | A | 228 | 38.080 | 61.989 | 52.853 | 1.00 | 55.66 | C |
| ATOM | 1715 | CB | ASN | A | 228 | 39.371 | 62.445 | 52.173 | 1.00 | 54.01 | C |
| ATOM | 1716 | CG | ASN | A | 228 | 40.213 | 63.356 | 53.054 | 1.00 | 53.22 | C |
| ATOM | 1717 | OD1 | ASN | A | 228 | 39.691 | 64.218 | 53.753 | 1.00 | 51.69 | O |
| ATOM | 1718 | ND2 | ASN | A | 228 | 41.532 | 63.173 | 53.009 | 1.00 | 53.36 | N |
| ATOM | 1719 | C | ASN | A | 228 | 37.042 | 63.108 | 52.818 | 1.00 | 58.93 | C |
| ATOM | 1720 | O | ASN | A | 228 | 36.977 | 63.880 | 51.860 | 1.00 | 56.15 | O |
| ATOM | 1721 | N | ALA | A | 229 | 36.238 | 63.187 | 53.874 | 1.00 | 60.08 | N |
| ATOM | 1722 | CA | ALA | A | 229 | 35.181 | 64.188 | 53.992 | 1.00 | 61.21 | C |
| ATOM | 1723 | CB | ALA | A | 229 | 34.597 | 64.150 | 55.409 | 1.00 | 59.94 | C |
| ATOM | 1724 | C | ALA | A | 229 | 35.554 | 65.632 | 53.620 | 1.00 | 62.72 | C |
| ATOM | 1725 | O | ALA | A | 229 | 34.948 | 66.214 | 52.720 | 1.00 | 59.29 | O |
| ATOM | 1726 | N | ALA | A | 230 | 36.532 | 66.205 | 54.320 | 1.00 | 64.91 | N |
| ATOM | 1727 | CA | ALA | A | 230 | 36.949 | 67.584 | 54.076 | 1.00 | 66.74 | C |
| ATOM | 1728 | CB | ALA | A | 230 | 37.750 | 68.110 | 55.262 | 1.00 | 63.84 | C |
| ATOM | 1729 | C | ALA | A | 230 | 37.736 | 67.788 | 52.778 | 1.00 | 67.75 | C |
| ATOM | 1730 | O | ALA | A | 230 | 37.627 | 68.839 | 52.147 | 1.00 | 69.40 | O |
| ATOM | 1731 | N | ASN | A | 231 | 38.528 | 66.794 | 52.380 | 1.00 | 67.53 | N |
| ATOM | 1732 | CA | ASN | A | 231 | 39.320 | 66.903 | 51.157 | 1.00 | 67.72 | C |
| ATOM | 1733 | CB | ASN | A | 231 | 40.803 | 67.074 | 51.507 | 1.00 | 67.59 | C |
| ATOM | 1734 | CG | ASN | A | 231 | 41.075 | 68.350 | 52.283 | 1.00 | 70.06 | C |
| ATOM | 1735 | OD1 | ASN | A | 231 | 40.753 | 68.462 | 53.467 | 1.00 | 67.95 | O |
| ATOM | 1736 | ND2 | ASN | A | 231 | 41.664 | 69.326 | 51.611 | 1.00 | 72.56 | N |
| ATOM | 1737 | C | ASN | A | 231 | 39.133 | 65.718 | 50.222 | 1.00 | 68.58 | C |
| ATOM | 1738 | O | ASN | A | 231 | 40.013 | 64.879 | 50.062 | 1.00 | 63.55 | O |
| ATOM | 1739 | N | SER | A | 232 | 37.963 | 65.690 | 49.598 | 1.00 | 72.53 | N |
| ATOM | 1740 | CA | SER | A | 232 | 37.541 | 64.638 | 48.676 | 1.00 | 77.35 | C |
| ATOM | 1741 | CB | SER | A | 232 | 36.784 | 65.263 | 47.503 | 1.00 | 75.62 | C |
| ATOM | 1742 | OG | SER | A | 232 | 36.319 | 64.271 | 46.604 | 1.00 | 71.95 | O |
| ATOM | 1743 | C | SER | A | 232 | 38.543 | 63.615 | 48.119 | 1.00 | 80.26 | C |
| ATOM | 1744 | O | SER | A | 232 | 38.251 | 62.420 | 48.122 | 1.00 | 83.05 | O |
| ATOM | 1745 | N | LYS | A | 233 | 39.708 | 64.042 | 47.644 | 1.00 | 83.37 | N |
| ATOM | 1746 | CA | LYS | A | 233 | 40.632 | 63.061 | 47.073 | 1.00 | 87.98 | C |
| ATOM | 1747 | CB | LYS | A | 233 | 40.720 | 63.253 | 45.553 | 1.00 | 90.59 | C |
| ATOM | 1748 | CG | LYS | A | 233 | 39.383 | 63.200 | 44.816 | 1.00 | 94.24 | C |
| ATOM | 1749 | CD | LYS | A | 233 | 38.829 | 61.786 | 44.670 | 1.00 | 95.91 | C |
| ATOM | 1750 | CE | LYS | A | 233 | 37.484 | 61.806 | 43.938 | 1.00 | 96.81 | C |
| ATOM | 1751 | NZ | LYS | A | 233 | 36.920 | 60.443 | 43.706 | 1.00 | 98.08 | N |
| ATOM | 1752 | C | LYS | A | 233 | 42.047 | 62.971 | 47.650 | 1.00 | 88.40 | C |
| ATOM | 1753 | O | LYS | A | 233 | 42.917 | 62.320 | 47.060 | 1.00 | 88.98 | O |
| ATOM | 1754 | N | GLN | A | 234 | 42.293 | 63.607 | 48.791 | 1.00 | 86.91 | N |
| ATOM | 1755 | CA | GLN | A | 234 | 43.617 | 63.529 | 49.402 | 1.00 | 84.61 | C |
| ATOM | 1756 | CB | GLN | A | 234 | 43.820 | 64.654 | 50.419 | 1.00 | 87.93 | C |
| ATOM | 1757 | CG | GLN | A | 234 | 43.954 | 66.042 | 49.849 | 1.00 | 95.63 | C |
| ATOM | 1758 | CD | GLN | A | 234 | 44.452 | 67.030 | 50.892 | 1.00 | 101.70 | C |
| ATOM | 1759 | OE1 | GLN | A | 234 | 44.552 | 68.230 | 50.630 | 1.00 | 104.69 | O |
| ATOM | 1760 | NE2 | GLN | A | 234 | 44.773 | 66.526 | 52.085 | 1.00 | 104.07 | N |
| ATOM | 1761 | C | GLN | A | 234 | 43.753 | 62.195 | 50.129 | 1.00 | 80.88 | C |
| ATOM | 1762 | O | GLN | A | 234 | 42.801 | 61.719 | 50.755 | 1.00 | 78.60 | O |
| ATOM | 1763 | N | PRO | A | 235 | 44.934 | 61.563 | 50.042 | 1.00 | 77.90 | N |
| ATOM | 1764 | CD | PRO | A | 235 | 46.011 | 61.774 | 49.061 | 1.00 | 76.60 | C |
| ATOM | 1765 | CA | PRO | A | 235 | 45.117 | 60.286 | 50.736 | 1.00 | 73.83 | C |
| ATOM | 1766 | CB | PRO | A | 235 | 46.506 | 59.843 | 50.287 | 1.00 | 74.44 | C |
| ATOM | 1767 | CG | PRO | A | 235 | 46.562 | 60.376 | 48.892 | 1.00 | 74.79 | C |
| ATOM | 1768 | C | PRO | A | 235 | 45.036 | 60.521 | 52.243 | 1.00 | 70.36 | C |
| ATOM | 1769 | O | PRO | A | 235 | 45.463 | 61.566 | 52.751 | 1.00 | 69.43 | O |
| ATOM | 1770 | N | ILE | A | 236 | 44.490 | 59.542 | 52.954 | 1.00 | 64.49 | N |
| ATOM | 1771 | CA | ILE | A | 236 | 44.324 | 59.652 | 54.392 | 1.00 | 58.44 | C |
| ATOM | 1772 | CB | ILE | A | 236 | 43.105 | 58.800 | 54.826 | 1.00 | 55.69 | C |
| ATOM | 1773 | CG2 | ILE | A | 236 | 42.983 | 58.771 | 56.343 | 1.00 | 48.13 | C |
| ATOM | 1774 | CG1 | ILE | A | 236 | 41.845 | 59.395 | 54.193 | 1.00 | 52.56 | C |
| ATOM | 1775 | CD1 | ILE | A | 236 | 40.613 | 58.522 | 54.234 | 1.00 | 50.82 | C |
| ATOM | 1776 | C | ILE | A | 236 | 45.591 | 59.279 | 55.164 | 1.00 | 56.27 | C |
| ATOM | 1777 | O | ILE | A | 236 | 46.097 | 58.161 | 55.053 | 1.00 | 54.65 | O |
| ATOM | 1778 | N | HIS | A | 237 | 46.113 | 60.262 | 55.899 | 1.00 | 54.64 | N |
| ATOM | 1779 | CA | HIS | A | 237 | 47.315 | 60.115 | 56.728 | 1.00 | 57.07 | C |
| ATOM | 1780 | CB | HIS | A | 237 | 48.426 | 61.085 | 56.291 | 1.00 | 62.64 | C |
| ATOM | 1781 | CG | HIS | A | 237 | 49.091 | 60.706 | 55.009 | 1.00 | 73.23 | C |
| ATOM | 1782 | CD2 | HIS | A | 237 | 48.913 | 61.162 | 53.744 | 1.00 | 76.99 | C |
| ATOM | 1783 | ND1 | HIS | A | 237 | 50.048 | 59.718 | 54.925 | 1.00 | 79.19 | N |
| ATOM | 1784 | CE1 | HIS | A | 237 | 50.432 | 59.579 | 53.667 | 1.00 | 79.37 | C |
| ATOM | 1785 | NE2 | HIS | A | 237 | 49.756 | 60.446 | 52.932 | 1.00 | 79.23 | N |
| ATOM | 1786 | C | HIS | A | 237 | 46.925 | 60.468 | 58.151 | 1.00 | 53.74 | C |
| ATOM | 1787 | O | HIS | A | 237 | 45.952 | 61.156 | 58.373 | 1.00 | 52.78 | O |
| ATOM | 1788 | N | MET | A | 238 | 47.709 | 60.021 | 59.115 | 1.00 | 49.75 | N |
| ATOM | 1789 | CA | MET | A | 238 | 47.449 | 60.332 | 60.511 | 1.00 | 47.80 | C |
| ATOM | 1790 | CB | MET | A | 238 | 46.345 | 59.434 | 61.083 | 1.00 | 41.01 | C |
| ATOM | 1791 | CG | MET | A | 238 | 46.704 | 57.947 | 61.198 | 1.00 | 42.77 | C |
| ATOM | 1792 | SD | MET | A | 238 | 45.394 | 56.928 | 62.010 | 1.00 | 45.73 | S |
| ATOM | 1793 | CE | MET | A | 238 | 45.278 | 57.744 | 63.593 | 1.00 | 37.92 | C |
| ATOM | 1794 | C | MET | A | 238 | 48.728 | 60.118 | 61.298 | 1.00 | 47.70 | C |
| ATOM | 1795 | O | MET | A | 238 | 49.730 | 59.643 | 60.757 | 1.00 | 41.57 | O |
| ATOM | 1796 | N | VAL | A | 239 | 48.695 | 60.514 | 62.563 | 1.00 | 46.31 | N |
| ATOM | 1797 | CA | VAL | A | 239 | 49.818 | 60.303 | 63.463 | 1.00 | 48.05 | C |
| ATOM | 1798 | CB | VAL | A | 239 | 50.005 | 61.500 | 64.425 | 1.00 | 48.85 | C |
| ATOM | 1799 | CG1 | VAL | A | 239 | 51.206 | 61.269 | 65.324 | 1.00 | 45.00 | C |
| ATOM | 1800 | CG2 | VAL | A | 239 | 50.148 | 62.779 | 63.630 | 1.00 | 47.20 | C |
| ATOM | 1801 | C | VAL | A | 239 | 49.392 | 59.063 | 64.265 | 1.00 | 50.50 | C |
| ATOM | 1802 | O | VAL | A | 239 | 48.350 | 59.072 | 64.929 | 1.00 | 49.70 | O |
| ATOM | 1803 | N | TYR | A | 240 | 50.174 | 57.992 | 64.188 | 1.00 | 45.76 | N |
| ATOM | 1804 | CA | TYR | A | 240 | 49.820 | 56.788 | 64.921 | 1.00 | 43.44 | C |
| ATOM | 1805 | CB | TYR | A | 240 | 48.645 | 56.075 | 64.206 | 1.00 | 39.98 | C |
| ATOM | 1806 | CG | TYR | A | 240 | 47.775 | 55.274 | 65.129 | 1.00 | 40.17 | C |
| ATOM | 1807 | CD1 | TYR | A | 240 | 47.727 | 53.870 | 65.033 | 1.00 | 37.13 | C |
| ATOM | 1808 | CE1 | TYR | A | 240 | 46.969 | 53.096 | 65.935 | 1.00 | 35.78 | C |
| ATOM | 1809 | CD2 | TYR | A | 240 | 47.040 | 55.902 | 66.139 | 1.00 | 28.47 | C |
| ATOM | 1810 | CE2 | TYR | A | 240 | 46.273 | 55.138 | 67.044 | 1.00 | 37.15 | C |
| ATOM | 1811 | CZ | TYR | A | 240 | 46.250 | 53.739 | 66.936 | 1.00 | 36.09 | C |
| ATOM | 1812 | OH | TYR | A | 240 | 45.526 | 52.995 | 67.822 | 1.00 | 35.34 | O |
| ATOM | 1813 | C | TYR | A | 240 | 51.018 | 55.840 | 65.076 | 1.00 | 43.90 | C |
| ATOM | 1814 | O | TYR | A | 240 | 52.107 | 56.112 | 64.597 | 1.00 | 40.90 | O |
| ATOM | 1815 | N | VAL | A | 241 | 50.816 | 54.748 | 65.804 | 1.00 | 43.98 | N |
| ATOM | 1816 | CA | VAL | A | 241 | 51.847 | 53.731 | 65.970 | 1.00 | 43.51 | C |
| ATOM | 1817 | CB | VAL | A | 241 | 51.862 | 53.120 | 67.391 | 1.00 | 42.83 | C |
| ATOM | 1818 | CG1 | VAL | A | 241 | 53.063 | 52.194 | 67.534 | 1.00 | 39.22 | C |
| ATOM | 1819 | CG2 | VAL | A | 241 | 51.916 | 54.218 | 68.431 | 1.00 | 43.95 | C |
| ATOM | 1820 | C | VAL | A | 241 | 51.438 | 52.656 | 64.966 | 1.00 | 46.24 | C |
| ATOM | 1821 | O | VAL | A | 241 | 50.567 | 51.826 | 65.240 | 1.00 | 42.02 | O |
| ATOM | 1822 | N | PRO | A | 242 | 52.062 | 52.668 | 63.777 | 1.00 | 46.93 | N |
| ATOM | 1823 | CD | PRO | A | 242 | 53.105 | 53.605 | 63.323 | 1.00 | 44.50 | C |
| ATOM | 1824 | CA | PRO | A | 242 | 51.740 | 51.691 | 62.733 | 1.00 | 47.04 | C |
| ATOM | 1825 | CB | PRO | A | 242 | 52.688 | 52.077 | 61.586 | 1.00 | 46.34 | C |
| ATOM | 1826 | CG | PRO | A | 242 | 53.788 | 52.816 | 62.257 | 1.00 | 47.66 | C |
| ATOM | 1827 | C | PRO | A | 242 | 51.753 | 50.199 | 63.075 | 1.00 | 47.85 | C |
| ATOM | 1828 | O | PRO | A | 242 | 50.922 | 49.448 | 62.555 | 1.00 | 44.48 | O |
| ATOM | 1829 | N | SER | A | 243 | 52.676 | 49.761 | 63.931 | 1.00 | 45.50 | N |
| ATOM | 1830 | CA | SER | A | 243 | 52.708 | 48.338 | 64.300 | 1.00 | 44.24 | C |
| ATOM | 1831 | CB | SER | A | 243 | 53.938 | 48.015 | 65.165 | 1.00 | 39.45 | C |
| ATOM | 1832 | OG | SER | A | 243 | 53.757 | 48.414 | 66.497 | 1.00 | 44.74 | O |
| ATOM | 1833 | C | SER | A | 243 | 51.401 | 47.948 | 65.044 | 1.00 | 42.31 | C |
| ATOM | 1834 | O | SER | A | 243 | 50.920 | 46.815 | 64.915 | 1.00 | 35.97 | O |
| ATOM | 1835 | N | HIS | A | 244 | 50.837 | 48.888 | 65.810 | 1.00 | 40.27 | N |
| ATOM | 1836 | CA | HIS | A | 244 | 49.593 | 48.637 | 66.540 | 1.00 | 41.82 | C |
| ATOM | 1837 | CB | HIS | A | 244 | 49.245 | 49.796 | 67.494 | 1.00 | 43.38 | C |
| ATOM | 1838 | CG | HIS | A | 244 | 50.094 | 49.884 | 68.721 | 1.00 | 49.85 | C |
| ATOM | 1839 | CD2 | HIS | A | 244 | 51.243 | 49.245 | 69.075 | 1.00 | 51.08 | C |
| ATOM | 1840 | ND1 | HIS | A | 244 | 49.804 | 50.736 | 69.764 | 1.00 | 51.24 | N |
| ATOM | 1841 | CE1 | HIS | A | 244 | 50.726 | 50.625 | 70.704 | 1.00 | 48.66 | C |
| ATOM | 1842 | NE2 | HIS | A | 244 | 51.610 | 49.724 | 70.304 | 1.00 | 51.09 | N |
| ATOM | 1843 | C | HIS | A | 244 | 48.414 | 48.462 | 65.549 | 1.00 | 41.54 | C |
| ATOM | 1844 | O | HIS | A | 244 | 47.578 | 47.549 | 65.718 | 1.00 | 34.86 | O |
| ATOM | 1845 | N | LEU | A | 245 | 48.353 | 49.332 | 64.530 | 1.00 | 39.59 | N |
| ATOM | 1846 | CA | LEU | A | 245 | 47.281 | 49.301 | 63.538 | 1.00 | 42.69 | C |
| ATOM | 1847 | CB | LEU | A | 245 | 47.366 | 50.532 | 62.625 | 1.00 | 43.02 | C |
| ATOM | 1848 | CG | LEU | A | 245 | 46.311 | 50.576 | 61.523 | 1.00 | 43.37 | C |
| ATOM | 1849 | CD1 | LEU | A | 245 | 44.909 | 50.603 | 62.128 | 1.00 | 35.16 | C |
| ATOM | 1850 | CD2 | LEU | A | 245 | 46.539 | 51.813 | 60.659 | 1.00 | 45.06 | C |
| ATOM | 1851 | C | LEU | A | 245 | 47.345 | 48.026 | 62.711 | 1.00 | 42.59 | C |
| ATOM | 1852 | O | LEU | A | 245 | 46.321 | 47.437 | 62.375 | 1.00 | 36.98 | O |
| ATOM | 1853 | N | TYR | A | 246 | 48.573 | 47.632 | 62.390 | 1.00 | 45.15 | N |
| ATOM | 1854 | CA | TYR | A | 246 | 48.871 | 46.421 | 61.635 | 1.00 | 47.24 | C |
| ATOM | 1855 | CB | TYR | A | 246 | 50.386 | 46.252 | 61.509 | 1.00 | 44.97 | C |
| ATOM | 1856 | CG | TYR | A | 246 | 50.786 | 45.059 | 60.661 | 1.00 | 45.92 | C |
| ATOM | 1857 | CD1 | TYR | A | 246 | 50.930 | 45.176 | 59.273 | 1.00 | 46.30 | C |
| ATOM | 1858 | CE1 | TYR | A | 246 | 51.216 | 44.076 | 58.485 | 1.00 | 45.15 | C |
| ATOM | 1859 | CD2 | TYR | A | 246 | 50.947 | 43.796 | 61.236 | 1.00 | 45.13 | C |
| ATOM | 1860 | CE2 | TYR | A | 246 | 51.225 | 42.678 | 60.453 | 1.00 | 44.58 | C |
| ATOM | 1861 | CZ | TYR | A | 246 | 51.356 | 42.830 | 59.080 | 1.00 | 47.10 | C |
| ATOM | 1862 | OH | TYR | A | 246 | 51.584 | 41.716 | 58.312 | 1.00 | 48.55 | O |
| ATOM | 1863 | C | TYR | A | 246 | 48.308 | 45.195 | 62.364 | 1.00 | 47.26 | C |
| ATOM | 1864 | O | TYR | A | 246 | 47.640 | 44.351 | 61.778 | 1.00 | 44.94 | O |
| ATOM | 1865 | N | HIS | A | 247 | 48.606 | 45.107 | 63.653 | 1.00 | 47.86 | N |
| ATOM | 1866 | CA | HIS | A | 247 | 48.155 | 44.011 | 64.491 | 1.00 | 50.58 | C |
| ATOM | 1867 | CB | HIS | A | 247 | 48.620 | 44.238 | 65.937 | 1.00 | 57.25 | C |
| ATOM | 1868 | CG | HIS | A | 247 | 48.253 | 43.130 | 66.879 | 1.00 | 68.14 | C |
| ATOM | 1869 | CD2 | HIS | A | 247 | 47.102 | 42.877 | 67.547 | 1.00 | 71.96 | C |
| ATOM | 1870 | ND1 | HIS | A | 247 | 49.123 | 42.112 | 67.213 | 1.00 | 73.47 | N |
| ATOM | 1871 | CE1 | HIS | A | 247 | 48.521 | 41.280 | 68.048 | 1.00 | 73.38 | C |
| ATOM | 1872 | NE2 | HIS | A | 247 | 47.297 | 41.720 | 68.267 | 1.00 | 73.64 | N |
| ATOM | 1873 | C | HIS | A | 247 | 46.644 | 43.838 | 64.454 | 1.00 | 48.19 | C |
| ATOM | 1874 | O | HIS | A | 247 | 46.149 | 42.742 | 64.237 | 1.00 | 45.12 | O |
| ATOM | 1875 | N | MET | A | 248 | 45.903 | 44.924 | 64.658 | 1.00 | 46.39 | N |
| ATOM | 1876 | CA | MET | A | 248 | 44.430 | 44.858 | 64.645 | 1.00 | 45.21 | C |
| ATOM | 1877 | CB | MET | A | 248 | 43.811 | 46.203 | 65.047 | 1.00 | 39.23 | C |
| ATOM | 1878 | CG | MET | A | 248 | 44.327 | 46.747 | 66.337 | 1.00 | 43.55 | C |
| ATOM | 1879 | SD | MET | A | 248 | 43.494 | 48.269 | 66.888 | 1.00 | 40.58 | S |
| ATOM | 1880 | CE | MET | A | 248 | 44.255 | 49.466 | 65.921 | 1.00 | 36.93 | C |
| ATOM | 1881 | C | MET | A | 248 | 43.900 | 44.442 | 63.273 | 1.00 | 42.84 | C |
| ATOM | 1882 | O | MET | A | 248 | 43.076 | 43.536 | 63.180 | 1.00 | 38.71 | O |
| ATOM | 1883 | N | LEU | A | 249 | 44.396 | 45.084 | 62.220 | 1.00 | 41.27 | N |
| ATOM | 1884 | CA | LEU | A | 249 | 43.965 | 44.750 | 60.868 | 1.00 | 41.68 | C |
| ATOM | 1885 | CB | LEU | A | 249 | 44.621 | 45.692 | 59.852 | 1.00 | 33.67 | C |
| ATOM | 1886 | CG | LEU | A | 249 | 44.056 | 47.127 | 59.840 | 1.00 | 40.97 | C |
| ATOM | 1887 | CD1 | LEU | A | 249 | 44.917 | 48.085 | 58.980 | 1.00 | 34.97 | C |
| ATOM | 1888 | CD2 | LEU | A | 249 | 42.605 | 47.067 | 59.323 | 1.00 | 36.90 | C |
| ATOM | 1889 | C | LEU | A | 249 | 44.249 | 43.283 | 60.510 | 1.00 | 40.15 | C |
| ATOM | 1890 | O | LEU | A | 249 | 43.397 | 42.592 | 59.946 | 1.00 | 38.27 | O |
| ATOM | 1891 | N | PHE | A | 250 | 45.435 | 42.813 | 60.868 | 1.00 | 38.86 | N |
| ATOM | 1892 | CA | PHE | A | 250 | 45.846 | 41.445 | 60.610 | 1.00 | 40.94 | C |
| ATOM | 1893 | CB | PHE | A | 250 | 47.300 | 41.250 | 61.076 | 1.00 | 43.11 | C |
| ATOM | 1894 | CG | PHE | A | 250 | 47.834 | 39.855 | 60.861 | 1.00 | 40.89 | C |
| ATOM | 1895 | CD1 | PHE | A | 250 | 47.970 | 39.335 | 59.576 | 1.00 | 40.60 | C |
| ATOM | 1896 | CD2 | PHE | A | 250 | 48.161 | 39.054 | 61.948 | 1.00 | 44.29 | C |
| ATOM | 1897 | CE1 | PHE | A | 250 | 48.410 | 38.052 | 59.364 | 1.00 | 40.67 | C |
| ATOM | 1898 | CE2 | PHE | A | 250 | 48.614 | 37.740 | 61.768 | 1.00 | 46.19 | C |
| ATOM | 1899 | CZ | PHE | A | 250 | 48.742 | 37.231 | 60.472 | 1.00 | 41.10 | C |
| ATOM | 1900 | C | PHE | A | 250 | 44.924 | 40.437 | 61.310 | 1.00 | 40.90 | C |
| ATOM | 1901 | O | PHE | A | 250 | 44.485 | 39.445 | 60.702 | 1.00 | 37.84 | O |
| ATOM | 1902 | N | GLU | A | 251 | 44.631 | 40.672 | 62.585 | 1.00 | 39.13 | N |
| ATOM | 1903 | CA | GLU | A | 251 | 43.748 | 39.747 | 63.296 | 1.00 | 44.17 | C |
| ATOM | 1904 | CB | GLU | A | 251 | 43.654 | 40.102 | 64.791 | 1.00 | 45.30 | C |
| ATOM | 1905 | CG | GLU | A | 251 | 44.972 | 39.941 | 65.568 | 1.00 | 59.86 | C |
| ATOM | 1906 | CD | GLU | A | 251 | 45.492 | 38.503 | 65.602 | 1.00 | 65.14 | C |
| ATOM | 1907 | OE1 | GLU | A | 251 | 44.734 | 37.598 | 66.029 | 1.00 | 61.95 | O |
| ATOM | 1908 | OE2 | GLU | A | 251 | 46.660 | 38.285 | 65.202 | 1.00 | 68.11 | O |
| ATOM | 1909 | C | GLU | A | 251 | 42.339 | 39.741 | 62.672 | 1.00 | 43.71 | C |
| ATOM | 1910 | O | GLU | A | 251 | 41.678 | 38.692 | 62.619 | 1.00 | 38.51 | O |
| ATOM | 1911 | N | LEU | A | 252 | 41.890 | 40.908 | 62.206 | 1.00 | 38.07 | N |
| ATOM | 1912 | CA | LEU | A | 252 | 40.576 | 41.007 | 61.607 | 1.00 | 42.47 | C |
| ATOM | 1913 | CB | LEU | A | 252 | 40.144 | 42.464 | 61.496 | 1.00 | 40.88 | C |
| ATOM | 1914 | CG | LEU | A | 252 | 39.831 | 43.132 | 62.850 | 1.00 | 42.27 | C |
| ATOM | 1915 | CD1 | LEU | A | 252 | 39.290 | 44.535 | 62.641 | 1.00 | 31.14 | C |
| ATOM | 1916 | CD2 | LEU | A | 252 | 38.834 | 42.271 | 63.608 | 1.00 | 38.04 | C |
| ATOM | 1917 | C | LEU | A | 252 | 40.557 | 40.338 | 60.246 | 1.00 | 41.43 | C |
| ATOM | 1918 | O | LEU | A | 252 | 39.616 | 39.635 | 59.927 | 1.00 | 40.81 | O |
| ATOM | 1919 | N | PHE | A | 253 | 41.608 | 40.539 | 59.457 | 1.00 | 42.22 | N |
| ATOM | 1920 | CA | PHE | A | 253 | 41.703 | 39.926 | 58.141 | 1.00 | 42.24 | C |
| ATOM | 1921 | CB | PHE | A | 253 | 42.963 | 40.402 | 57.418 | 1.00 | 40.99 | C |
| ATOM | 1922 | CG | PHE | A | 253 | 42.766 | 41.646 | 56.572 | 1.00 | 36.06 | C |
| ATOM | 1923 | CD1 | PHE | A | 253 | 41.771 | 41.678 | 55.584 | 1.00 | 32.32 | C |
| ATOM | 1924 | CD2 | PHE | A | 253 | 43.634 | 42.753 | 56.702 | 1.00 | 30.20 | C |
| ATOM | 1925 | CE1 | PHE | A | 253 | 41.643 | 42.818 | 54.702 | 1.00 | 32.27 | C |
| ATOM | 1926 | CE2 | PHE | A | 253 | 43.533 | 43.884 | 55.861 | 1.00 | 27.78 | C |
| ATOM | 1927 | CZ | PHE | A | 253 | 42.541 | 43.930 | 54.851 | 1.00 | 29.81 | C |
| ATOM | 1928 | C | PHE | A | 253 | 41.701 | 38.391 | 58.249 | 1.00 | 44.55 | C |
| ATOM | 1929 | O | PHE | A | 253 | 41.058 | 37.717 | 57.457 | 1.00 | 44.25 | O |
| ATOM | 1930 | N | LYS | A | 254 | 42.401 | 37.840 | 59.238 | 1.00 | 44.59 | N |
| ATOM | 1931 | CA | LYS | A | 254 | 42.455 | 36.387 | 59.447 | 1.00 | 45.62 | C |
| ATOM | 1932 | CB | LYS | A | 254 | 43.421 | 36.044 | 60.602 | 1.00 | 48.53 | C |
| ATOM | 1933 | CG | LYS | A | 254 | 44.897 | 35.893 | 60.206 | 1.00 | 51.97 | C |
| ATOM | 1934 | CD | LYS | A | 254 | 45.850 | 36.113 | 61.409 | 1.00 | 52.06 | C |
| ATOM | 1935 | CE | LYS | A | 254 | 45.603 | 35.145 | 62.564 | 1.00 | 51.87 | C |
| ATOM | 1936 | NZ | LYS | A | 254 | 46.412 | 35.458 | 63.780 | 1.00 | 44.26 | N |
| ATOM | 1937 | C | LYS | A | 254 | 41.082 | 35.777 | 59.747 | 1.00 | 46.47 | C |
| ATOM | 1938 | O | LYS | A | 254 | 40.793 | 34.669 | 59.311 | 1.00 | 43.53 | O |
| ATOM | 1939 | N | ASN | A | 255 | 40.258 | 36.489 | 60.522 | 1.00 | 44.72 | N |
| ATOM | 1940 | CA | ASN | A | 255 | 38.897 | 36.028 | 60.867 | 1.00 | 45.60 | C |
| ATOM | 1941 | CB | ASN | A | 255 | 38.274 | 36.873 | 61.984 | 1.00 | 42.99 | C |
| ATOM | 1942 | CG | ASN | A | 255 | 38.985 | 36.708 | 63.308 | 1.00 | 50.89 | C |
| ATOM | 1943 | OD1 | ASN | A | 255 | 39.540 | 35.655 | 63.583 | 1.00 | 53.85 | O |
| ATOM | 1944 | ND2 | ASN | A | 255 | 38.957 | 37.752 | 64.146 | 1.00 | 46.55 | N |
| ATOM | 1945 | C | ASN | A | 255 | 37.996 | 36.098 | 59.636 | 1.00 | 44.07 | C |
| ATOM | 1946 | O | ASN | A | 255 | 37.305 | 35.128 | 59.315 | 1.00 | 41.56 | O |
| ATOM | 1947 | N | ALA | A | 256 | 38.020 | 37.244 | 58.958 | 1.00 | 42.06 | N |
| ATOM | 1948 | CA | ALA | A | 256 | 37.231 | 37.449 | 57.769 | 1.00 | 48.15 | C |
| ATOM | 1949 | CB | ALA | A | 256 | 37.475 | 38.861 | 57.226 | 1.00 | 43.70 | C |
| ATOM | 1950 | C | ALA | A | 256 | 37.575 | 36.386 | 56.707 | 1.00 | 51.99 | C |
| ATOM | 1951 | O | ALA | A | 256 | 36.685 | 35.840 | 56.061 | 1.00 | 51.70 | O |
| ATOM | 1952 | N | MET | A | 257 | 38.860 | 36.087 | 56.528 | 1.00 | 52.79 | N |
| ATOM | 1953 | CA | MET | A | 257 | 39.266 | 35.095 | 55.528 | 1.00 | 53.61 | C |
| ATOM | 1954 | CB | MET | A | 257 | 40.801 | 35.118 | 55.336 | 1.00 | 54.33 | C |
| ATOM | 1955 | CG | MET | A | 257 | 41.257 | 36.302 | 54.493 | 1.00 | 58.90 | C |
| ATOM | 1956 | SD | MET | A | 257 | 43.012 | 36.524 | 54.308 | 1.00 | 61.36 | S |
| ATOM | 1957 | CE | MET | A | 257 | 43.036 | 38.068 | 53.377 | 1.00 | 50.40 | C |
| ATOM | 1958 | C | MET | A | 257 | 38.794 | 33.695 | 55.874 | 1.00 | 51.52 | C |
| ATOM | 1959 | O | MET | A | 257 | 38.222 | 33.003 | 55.037 | 1.00 | 46.59 | O |
| ATOM | 1960 | N | ARG | A | 258 | 39.042 | 33.282 | 57.110 | 1.00 | 52.43 | N |
| ATOM | 1961 | CA | ARG | A | 258 | 38.640 | 31.960 | 57.551 | 1.00 | 53.89 | C |
| ATOM | 1962 | CB | ARG | A | 258 | 38.951 | 31.804 | 59.034 | 1.00 | 55.42 | C |
| ATOM | 1963 | CG | ARG | A | 258 | 39.155 | 30.371 | 59.452 | 1.00 | 64.38 | C |
| ATOM | 1964 | CD | ARG | A | 258 | 38.098 | 29.867 | 60.385 | 1.00 | 68.56 | C |
| ATOM | 1965 | NE | ARG | A | 258 | 38.377 | 30.194 | 61.781 | 1.00 | 76.66 | N |
| ATOM | 1966 | CZ | ARG | A | 258 | 37.724 | 29.659 | 62.812 | 1.00 | 82.19 | C |
| ATOM | 1967 | NH1 | ARG | A | 258 | 36.758 | 28.768 | 62.602 | 1.00 | 83.02 | N |
| ATOM | 1968 | NH2 | ARG | A | 258 | 38.030 | 30.015 | 64.055 | 1.00 | 84.76 | N |
| ATOM | 1969 | C | ARG | A | 258 | 37.135 | 31.786 | 57.300 | 1.00 | 56.87 | C |
| ATOM | 1970 | O | ARG | A | 258 | 36.714 | 30.882 | 56.591 | 1.00 | 54.38 | O |
| ATOM | 1971 | N | ALA | A | 259 | 36.333 | 32.686 | 57.860 | 1.00 | 54.19 | N |
| ATOM | 1972 | CA | ALA | A | 259 | 34.883 | 32.632 | 57.709 | 1.00 | 52.98 | C |
| ATOM | 1973 | CB | ALA | A | 259 | 34.216 | 33.832 | 58.435 | 1.00 | 47.92 | C |
| ATOM | 1974 | C | ALA | A | 259 | 34.426 | 32.572 | 56.253 | 1.00 | 49.65 | C |
| ATOM | 1975 | O | ALA | A | 259 | 33.637 | 31.710 | 55.894 | 1.00 | 49.75 | O |
| ATOM | 1976 | N | THR | A | 260 | 34.925 | 33.482 | 55.425 | 1.00 | 47.56 | N |
| ATOM | 1977 | CA | THR | A | 260 | 34.545 | 33.533 | 54.017 | 1.00 | 52.57 | C |
| ATOM | 1978 | CB | THR | A | 260 | 35.248 | 34.686 | 53.258 | 1.00 | 54.00 | C |
| ATOM | 1979 | OG1 | THR | A | 260 | 34.918 | 35.940 | 53.861 | 1.00 | 53.47 | O |
| ATOM | 1980 | CG2 | THR | A | 260 | 34.797 | 34.709 | 51.798 | 1.00 | 54.43 | C |
| ATOM | 1981 | C | THR | A | 260 | 34.868 | 32.239 | 53.284 | 1.00 | 55.98 | C |
| ATOM | 1982 | O | THR | A | 260 | 34.052 | 31.734 | 52.512 | 1.00 | 54.37 | O |
| ATOM | 1983 | N | VAL | A | 261 | 36.058 | 31.696 | 53.526 | 1.00 | 58.06 | N |
| ATOM | 1984 | CA | VAL | A | 261 | 36.445 | 30.457 | 52.862 | 1.00 | 58.73 | C |
| ATOM | 1985 | CB | VAL | A | 261 | 37.978 | 30.160 | 53.042 | 1.00 | 57.94 | C |
| ATOM | 1986 | CG1 | VAL | A | 261 | 38.293 | 28.719 | 52.668 | 1.00 | 57.09 | C |
| ATOM | 1987 | CG2 | VAL | A | 261 | 38.798 | 31.112 | 52.164 | 1.00 | 51.92 | C |
| ATOM | 1988 | C | VAL | A | 261 | 35.620 | 29.269 | 53.355 | 1.00 | 58.60 | C |
| ATOM | 1989 | O | VAL | A | 261 | 35.035 | 28.551 | 52.564 | 1.00 | 60.03 | O |
| ATOM | 1990 | N | GLU | A | 262 | 35.550 | 29.078 | 54.658 | 1.00 | 57.87 | N |
| ATOM | 1991 | CA | GLU | A | 262 | 34.812 | 27.952 | 55.181 | 1.00 | 62.34 | C |
| ATOM | 1992 | CB | GLU | A | 262 | 35.062 | 27.832 | 56.679 | 1.00 | 62.03 | C |
| ATOM | 1993 | CG | GLU | A | 262 | 36.525 | 27.588 | 57.018 | 1.00 | 64.14 | C |
| ATOM | 1994 | CD | GLU | A | 262 | 36.742 | 27.281 | 58.481 | 1.00 | 68.80 | C |
| ATOM | 1995 | OE1 | GLU | A | 262 | 37.898 | 27.015 | 58.869 | 1.00 | 71.01 | O |
| ATOM | 1996 | OE2 | GLU | A | 262 | 35.761 | 27.305 | 59.248 | 1.00 | 73.00 | O |
| ATOM | 1997 | C | GLU | A | 262 | 33.310 | 27.988 | 54.895 | 1.00 | 65.04 | C |
| ATOM | 1998 | O | GLU | A | 262 | 32.665 | 26.943 | 54.809 | 1.00 | 67.25 | O |
| ATOM | 1999 | N | SER | A | 263 | 32.744 | 29.176 | 54.727 | 1.00 | 66.39 | N |
| ATOM | 2000 | CA | SER | A | 263 | 31.315 | 29.279 | 54.460 | 1.00 | 66.84 | C |
| ATOM | 2001 | CB | SER | A | 263 | 30.765 | 30.599 | 55.018 | 1.00 | 64.65 | C |
| ATOM | 2002 | OG | SER | A | 263 | 31.268 | 31.732 | 54.328 | 1.00 | 57.86 | O |
| ATOM | 2003 | C | SER | A | 263 | 30.984 | 29.162 | 52.971 | 1.00 | 69.21 | C |
| ATOM | 2004 | O | SER | A | 263 | 29.819 | 29.171 | 52.581 | 1.00 | 70.10 | O |
| ATOM | 2005 | N | HIS | A | 264 | 32.006 | 29.034 | 52.136 | 1.00 | 71.00 | N |
| ATOM | 2006 | CA | HIS | A | 264 | 31.794 | 28.917 | 50.692 | 1.00 | 72.82 | C |
| ATOM | 2007 | CB | HIS | A | 264 | 32.202 | 30.212 | 49.986 | 1.00 | 71.20 | C |
| ATOM | 2008 | CG | HIS | A | 264 | 31.330 | 31.384 | 50.314 | 1.00 | 74.02 | C |
| ATOM | 2009 | CD2 | HIS | A | 264 | 31.130 | 32.053 | 51.474 | 1.00 | 73.39 | C |
| ATOM | 2010 | ND1 | HIS | A | 264 | 30.574 | 32.035 | 49.364 | 1.00 | 74.57 | N |
| ATOM | 2011 | CE1 | HIS | A | 264 | 29.950 | 33.057 | 49.922 | 1.00 | 72.15 | C |
| ATOM | 2012 | NE2 | HIS | A | 264 | 30.272 | 33.090 | 51.203 | 1.00 | 74.10 | N |
| ATOM | 2013 | C | HIS | A | 264 | 32.574 | 27.760 | 50.082 | 1.00 | 74.03 | C |
| ATOM | 2014 | O | HIS | A | 264 | 32.991 | 27.831 | 48.932 | 1.00 | 74.17 | O |
| ATOM | 2015 | N | GLU | A | 265 | 32.765 | 26.691 | 50.847 | 1.00 | 76.56 | N |
| ATOM | 2016 | CA | GLU | A | 265 | 33.516 | 25.542 | 50.357 | 1.00 | 79.42 | C |
| ATOM | 2017 | CB | GLU | A | 265 | 33.905 | 24.626 | 51.517 | 1.00 | 79.51 | C |
| ATOM | 2018 | CG | GLU | A | 265 | 34.812 | 25.274 | 52.514 | 1.00 | 87.55 | C |
| ATOM | 2019 | CD | GLU | A | 265 | 35.331 | 24.283 | 53.526 | 1.00 | 91.28 | C |
| ATOM | 2020 | OE1 | GLU | A | 265 | 34.500 | 23.529 | 54.082 | 1.00 | 91.44 | O |
| ATOM | 2021 | OE2 | GLU | A | 265 | 36.564 | 24.258 | 53.769 | 1.00 | 94.53 | O |
| ATOM | 2022 | C | GLU | A | 265 | 32.753 | 24.731 | 49.328 | 1.00 | 80.07 | C |
| ATOM | 2023 | O | GLU | A | 265 | 33.038 | 23.543 | 49.142 | 1.00 | 80.64 | O |
| ATOM | 2024 | N | SER | A | 266 | 31.800 | 25.364 | 48.651 | 1.00 | 79.42 | N |
| ATOM | 2025 | CA | SER | A | 266 | 31.010 | 24.658 | 47.664 | 1.00 | 78.31 | C |
| ATOM | 2026 | CB | SER | A | 266 | 29.850 | 23.908 | 48.337 | 1.00 | 75.40 | C |
| ATOM | 2027 | OG | SER | A | 266 | 30.305 | 23.072 | 49.391 | 1.00 | 74.69 | O |
| ATOM | 2028 | C | SER | A | 266 | 30.445 | 25.632 | 46.659 | 1.00 | 79.91 | C |
| ATOM | 2029 | O | SER | A | 266 | 29.273 | 25.522 | 46.303 | 1.00 | 82.28 | O |
| ATOM | 2030 | N | SER | A | 267 | 31.257 | 26.575 | 46.185 | 1.00 | 81.33 | N |
| ATOM | 2031 | CA | SER | A | 267 | 30.740 | 27.547 | 45.220 | 1.00 | 85.42 | C |
| ATOM | 2032 | CB | SER | A | 267 | 29.500 | 28.230 | 45.813 | 1.00 | 86.14 | C |
| ATOM | 2033 | OG | SER | A | 267 | 29.714 | 28.569 | 47.175 | 1.00 | 85.22 | O |
| ATOM | 2034 | C | SER | A | 267 | 31.724 | 28.611 | 44.729 | 1.00 | 87.72 | C |
| ATOM | 2035 | O | SER | A | 267 | 32.681 | 28.307 | 44.013 | 1.00 | 87.00 | O |
| ATOM | 2036 | N | LEU | A | 268 | 31.466 | 29.863 | 45.107 | 1.00 | 91.03 | N |
| ATOM | 2037 | CA | LEU | A | 268 | 32.310 | 30.991 | 44.719 | 1.00 | 91.61 | C |
| ATOM | 2038 | CB | LEU | A | 268 | 31.512 | 31.935 | 43.816 | 1.00 | 92.58 | C |
| ATOM | 2039 | CG | LEU | A | 268 | 32.015 | 33.367 | 43.635 | 1.00 | 94.37 | C |
| ATOM | 2040 | CD1 | LEU | A | 268 | 31.833 | 33.798 | 42.192 | 1.00 | 94.22 | C |
| ATOM | 2041 | CD2 | LEU | A | 268 | 31.268 | 34.291 | 44.592 | 1.00 | 93.17 | C |
| ATOM | 2042 | C | LEU | A | 268 | 32.824 | 31.726 | 45.963 | 1.00 | 90.25 | C |
| ATOM | 2043 | O | LEU | A | 268 | 32.285 | 31.551 | 47.053 | 1.00 | 93.21 | O |
| ATOM | 2044 | N | ILE | A | 269 | 33.847 | 32.562 | 45.799 | 1.00 | 86.78 | N |
| ATOM | 2045 | CA | ILE | A | 269 | 34.444 | 33.278 | 46.931 | 1.00 | 81.12 | C |
| ATOM | 2046 | CB | ILE | A | 269 | 35.927 | 32.843 | 47.082 | 1.00 | 81.96 | C |
| ATOM | 2047 | CG2 | ILE | A | 269 | 36.769 | 33.399 | 45.922 | 1.00 | 85.32 | C |
| ATOM | 2048 | CG1 | ILE | A | 269 | 36.489 | 33.283 | 48.423 | 1.00 | 82.42 | C |
| ATOM | 2049 | CD1 | ILE | A | 269 | 37.857 | 32.696 | 48.706 | 1.00 | 81.87 | C |
| ATOM | 2050 | C | ILE | A | 269 | 34.345 | 34.813 | 46.850 | 1.00 | 75.84 | C |
| ATOM | 2051 | O | ILE | A | 269 | 35.031 | 35.459 | 46.054 | 1.00 | 74.96 | O |
| ATOM | 2052 | N | PRO | A | 271 | 33.478 | 35.422 | 47.684 | 1.00 | 71.12 | N |
| ATOM | 2053 | CD | PRO | A | 271 | 32.500 | 34.794 | 48.592 | 1.00 | 67.05 | C |
| ATOM | 2054 | CA | PRO | A | 271 | 33.324 | 36.890 | 47.669 | 1.00 | 66.98 | C |
| ATOM | 2055 | CB | PRO | A | 271 | 31.980 | 37.106 | 48.369 | 1.00 | 63.46 | C |
| ATOM | 2056 | CG | PRO | A | 271 | 31.949 | 35.986 | 49.358 | 1.00 | 66.21 | C |
| ATOM | 2057 | C | PRO | A | 271 | 34.497 | 37.612 | 48.359 | 1.00 | 64.03 | C |
| ATOM | 2058 | O | PRO | A | 271 | 35.092 | 37.087 | 49.304 | 1.00 | 63.55 | O |
| ATOM | 2059 | N | PRO | A | 272 | 34.847 | 38.827 | 47.889 | 1.00 | 60.17 | N |
| ATOM | 2060 | CD | PRO | A | 272 | 34.250 | 39.530 | 46.734 | 1.00 | 55.78 | C |
| ATOM | 2061 | CA | PRO | A | 272 | 35.957 | 39.611 | 48.458 | 1.00 | 57.99 | C |
| ATOM | 2062 | CB | PRO | A | 272 | 36.243 | 40.640 | 47.365 | 1.00 | 54.62 | C |
| ATOM | 2063 | CG | PRO | A | 272 | 34.862 | 40.927 | 46.841 | 1.00 | 53.23 | C |
| ATOM | 2064 | C | PRO | A | 272 | 35.712 | 40.309 | 49.799 | 1.00 | 56.90 | C |
| ATOM | 2065 | O | PRO | A | 272 | 34.579 | 40.474 | 50.247 | 1.00 | 56.07 | O |
| ATOM | 2066 | N | ILE | A | 273 | 36.806 | 40.724 | 50.424 | 1.00 | 54.97 | N |
| ATOM | 2067 | CA | ILE | A | 273 | 36.745 | 41.454 | 51.669 | 1.00 | 50.78 | C |
| ATOM | 2068 | CB | ILE | A | 273 | 37.863 | 40.997 | 52.621 | 1.00 | 48.18 | C |
| ATOM | 2069 | CG2 | ILE | A | 273 | 37.884 | 41.889 | 53.866 | 1.00 | 50.36 | C |
| ATOM | 2070 | CG1 | ILE | A | 273 | 37.632 | 39.518 | 52.978 | 1.00 | 44.21 | C |
| ATOM | 2071 | CD1 | ILE | A | 273 | 38.624 | 38.890 | 53.957 | 1.00 | 43.83 | C |
| ATOM | 2072 | C | ILE | A | 273 | 36.876 | 42.941 | 51.330 | 1.00 | 50.05 | C |
| ATOM | 2073 | O | ILE | A | 273 | 37.842 | 43.354 | 50.719 | 1.00 | 49.99 | O |
| ATOM | 2074 | N | LYS | A | 274 | 35.875 | 43.732 | 51.699 | 1.00 | 51.53 | N |
| ATOM | 2075 | CA | LYS | A | 274 | 35.888 | 45.164 | 51.418 | 1.00 | 52.61 | C |
| ATOM | 2076 | CB | LYS | A | 274 | 34.537 | 45.626 | 50.880 | 1.00 | 55.42 | C |
| ATOM | 2077 | CG | LYS | A | 274 | 34.062 | 44.839 | 49.701 | 1.00 | 62.47 | C |
| ATOM | 2078 | CD | LYS | A | 274 | 32.744 | 45.375 | 49.191 | 1.00 | 66.15 | C |
| ATOM | 2079 | CE | LYS | A | 274 | 32.177 | 44.444 | 48.125 | 1.00 | 71.86 | C |
| ATOM | 2080 | NZ | LYS | A | 274 | 30.873 | 44.945 | 47.633 | 1.00 | 77.17 | N |
| ATOM | 2081 | C | LYS | A | 274 | 36.227 | 45.996 | 52.636 | 1.00 | 52.90 | C |
| ATOM | 2082 | O | LYS | A | 274 | 35.670 | 45.814 | 53.713 | 1.00 | 55.14 | O |
| ATOM | 2083 | N | VAL | A | 275 | 37.137 | 46.934 | 52.443 | 1.00 | 47.21 | N |
| ATOM | 2084 | CA | VAL | A | 275 | 37.558 | 47.791 | 53.520 | 1.00 | 44.02 | C |
| ATOM | 2085 | CB | VAL | A | 275 | 39.077 | 47.660 | 53.802 | 1.00 | 37.80 | C |
| ATOM | 2086 | CG1 | VAL | A | 275 | 39.561 | 48.847 | 54.640 | 1.00 | 33.80 | C |
| ATOM | 2087 | CG2 | VAL | A | 275 | 39.361 | 46.376 | 54.556 | 1.00 | 34.84 | C |
| ATOM | 2088 | C | VAL | A | 275 | 37.287 | 49.228 | 53.153 | 1.00 | 47.93 | C |
| ATOM | 2089 | O | VAL | A | 275 | 37.573 | 49.646 | 52.034 | 1.00 | 49.55 | O |
| ATOM | 2090 | N | MET | A | 276 | 36.724 | 49.978 | 54.093 | 1.00 | 47.27 | N |
| ATOM | 2091 | CA | MET | A | 276 | 36.483 | 51.382 | 53.867 | 1.00 | 46.59 | C |
| ATOM | 2092 | CB | MET | A | 276 | 35.008 | 51.731 | 53.910 | 1.00 | 51.89 | C |
| ATOM | 2093 | CG | MET | A | 276 | 34.798 | 53.232 | 53.718 | 1.00 | 57.23 | C |
| ATOM | 2094 | SD | MET | A | 276 | 33.080 | 53.762 | 53.583 | 1.00 | 68.24 | S |
| ATOM | 2095 | CE | MET | A | 276 | 33.050 | 54.969 | 54.894 | 1.00 | 69.04 | C |
| ATOM | 2096 | C | MET | A | 276 | 37.199 | 52.205 | 54.919 | 1.00 | 44.10 | C |
| ATOM | 2097 | O | MET | A | 276 | 37.116 | 51.929 | 56.118 | 1.00 | 41.58 | O |
| ATOM | 2098 | N | VAL | A | 277 | 37.916 | 53.222 | 54.458 | 1.00 | 40.59 | N |
| ATOM | 2099 | CA | VAL | A | 277 | 38.641 | 54.110 | 55.355 | 1.00 | 37.48 | C |
| ATOM | 2100 | CB | VAL | A | 277 | 40.169 | 54.146 | 55.008 | 1.00 | 35.15 | C |
| ATOM | 2101 | CG1 | VAL | A | 277 | 40.931 | 55.051 | 55.988 | 1.00 | 23.39 | C |
| ATOM | 2102 | CG2 | VAL | A | 277 | 40.751 | 52.691 | 55.048 | 1.00 | 26.72 | C |
| ATOM | 2103 | C | VAL | A | 277 | 38.009 | 55.486 | 55.210 | 1.00 | 38.82 | C |
| ATOM | 2104 | O | VAL | A | 277 | 38.111 | 56.118 | 54.166 | 1.00 | 41.24 | O |
| ATOM | 2105 | N | ALA | A | 278 | 37.344 | 55.925 | 56.272 | 1.00 | 36.94 | N |
| ATOM | 2106 | CA | ALA | A | 278 | 36.649 | 57.209 | 56.300 | 1.00 | 38.67 | C |
| ATOM | 2107 | CB | ALA | A | 278 | 35.156 | 57.009 | 56.662 | 1.00 | 27.53 | C |
| ATOM | 2108 | C | ALA | A | 278 | 37.283 | 58.185 | 57.300 | 1.00 | 39.63 | C |
| ATOM | 2109 | O | ALA | A | 278 | 37.571 | 57.833 | 58.444 | 1.00 | 40.73 | O |
| ATOM | 2110 | N | LEU | A | 279 | 37.471 | 59.425 | 56.874 | 1.00 | 40.13 | N |
| ATOM | 2111 | CA | LEU | A | 279 | 38.053 | 60.433 | 57.736 | 1.00 | 40.86 | C |
| ATOM | 2112 | CB | LEU | A | 279 | 39.320 | 60.980 | 57.093 | 1.00 | 37.57 | C |
| ATOM | 2113 | CG | LEU | A | 279 | 39.963 | 62.139 | 57.845 | 1.00 | 41.72 | C |
| ATOM | 2114 | CD1 | LEU | A | 279 | 40.367 | 61.724 | 59.245 | 1.00 | 38.29 | C |
| ATOM | 2115 | CD2 | LEU | A | 279 | 41.158 | 62.622 | 57.058 | 1.00 | 41.05 | C |
| ATOM | 2116 | C | LEU | A | 279 | 37.067 | 61.565 | 57.995 | 1.00 | 42.80 | C |
| ATOM | 2117 | O | LEU | A | 279 | 36.695 | 62.284 | 57.074 | 1.00 | 46.78 | O |
| ATOM | 2118 | N | GLY | A | 280 | 36.636 | 61.718 | 59.245 | 1.00 | 45.53 | N |
| ATOM | 2119 | CA | GLY | A | 280 | 35.701 | 62.775 | 59.581 | 1.00 | 43.77 | C |
| ATOM | 2120 | C | GLY | A | 280 | 36.408 | 63.869 | 60.359 | 1.00 | 47.22 | C |
| ATOM | 2121 | O | GLY | A | 280 | 37.630 | 63.942 | 60.308 | 1.00 | 45.46 | O |
| ATOM | 2122 | N | GLU | A | 281 | 35.660 | 64.706 | 61.078 | 1.00 | 49.64 | N |
| ATOM | 2123 | CA | GLU | A | 281 | 36.250 | 65.803 | 61.843 | 1.00 | 55.56 | C |
| ATOM | 2124 | CB | GLU | A | 281 | 35.191 | 66.863 | 62.180 | 1.00 | 59.53 | C |
| ATOM | 2125 | CG | GLU | A | 281 | 34.718 | 67.687 | 60.979 | 1.00 | 69.76 | C |
| ATOM | 2126 | CD | GLU | A | 281 | 35.864 | 68.319 | 60.171 | 1.00 | 73.61 | C |
| ATOM | 2127 | OE1 | GLU | A | 281 | 36.669 | 69.075 | 60.764 | 1.00 | 76.30 | O |
| ATOM | 2128 | OE2 | GLU | A | 281 | 35.949 | 68.058 | 58.943 | 1.00 | 75.16 | O |
| ATOM | 2129 | C | GLU | A | 281 | 36.979 | 65.396 | 63.127 | 1.00 | 55.39 | C |
| ATOM | 2130 | O | GLU | A | 281 | 37.785 | 66.170 | 63.641 | 1.00 | 55.84 | O |
| ATOM | 2131 | N | GLU | A | 282 | 36.673 | 64.220 | 63.670 | 1.00 | 54.69 | N |
| ATOM | 2132 | CA | GLU | A | 282 | 37.366 | 63.767 | 64.866 | 1.00 | 54.99 | C |
| ATOM | 2133 | CB | GLU | A | 282 | 36.604 | 64.164 | 66.139 | 1.00 | 55.21 | C |
| ATOM | 2134 | CG | GLU | A | 282 | 35.237 | 63.557 | 66.327 | 1.00 | 59.08 | C |
| ATOM | 2135 | CD | GLU | A | 282 | 34.627 | 63.955 | 67.667 | 1.00 | 64.58 | C |
| ATOM | 2136 | OE1 | GLU | A | 282 | 35.106 | 63.468 | 68.723 | 1.00 | 63.71 | O |
| ATOM | 2137 | OE2 | GLU | A | 282 | 33.667 | 64.762 | 67.661 | 1.00 | 64.50 | O |
| ATOM | 2138 | C | GLU | A | 282 | 37.658 | 62.268 | 64.852 | 1.00 | 52.04 | C |
| ATOM | 2139 | O | GLU | A | 282 | 38.525 | 61.787 | 65.575 | 1.00 | 50.73 | O |
| ATOM | 2140 | N | ASP | A | 283 | 36.943 | 61.541 | 64.003 | 1.00 | 47.64 | N |
| ATOM | 2141 | CA | ASP | A | 283 | 37.118 | 60.102 | 63.872 | 1.00 | 41.52 | C |
| ATOM | 2142 | CB | ASP | A | 283 | 35.784 | 59.338 | 63.900 | 1.00 | 38.89 | C |
| ATOM | 2143 | CG | ASP | A | 283 | 35.050 | 59.434 | 65.230 | 1.00 | 36.83 | C |
| ATOM | 2144 | OD1 | ASP | A | 283 | 35.574 | 60.043 | 66.206 | 1.00 | 36.16 | O |
| ATOM | 2145 | OD2 | ASP | A | 283 | 33.919 | 58.882 | 65.295 | 1.00 | 34.93 | O |
| ATOM | 2146 | C | ASP | A | 283 | 37.763 | 59.739 | 62.560 | 1.00 | 43.80 | C |
| ATOM | 2147 | O | ASP | A | 283 | 37.584 | 60.391 | 61.553 | 1.00 | 46.48 | O |
| ATOM | 2148 | N | LEU | A | 284 | 38.539 | 58.673 | 62.601 | 1.00 | 44.77 | N |
| ATOM | 2149 | CA | LEU | A | 284 | 39.172 | 58.091 | 61.424 | 1.00 | 41.01 | C |
| ATOM | 2150 | CB | LEU | A | 284 | 40.692 | 58.161 | 61.492 | 1.00 | 44.76 | C |
| ATOM | 2151 | CG | LEU | A | 284 | 41.312 | 58.080 | 60.089 | 1.00 | 45.46 | C |
| ATOM | 2152 | CD1 | LEU | A | 284 | 42.746 | 58.505 | 60.147 | 1.00 | 46.95 | C |
| ATOM | 2153 | CD2 | LEU | A | 284 | 41.169 | 56.663 | 59.496 | 1.00 | 46.70 | C |
| ATOM | 2154 | C | LEU | A | 284 | 38.695 | 56.657 | 61.601 | 1.00 | 39.60 | C |
| ATOM | 2155 | O | LEU | A | 284 | 39.082 | 55.961 | 62.531 | 1.00 | 37.26 | O |
| ATOM | 2156 | N | SER | A | 285 | 37.790 | 56.241 | 60.731 | 1.00 | 38.61 | N |
| ATOM | 2157 | CA | SER | A | 285 | 37.179 | 54.922 | 60.827 | 1.00 | 36.88 | C |
| ATOM | 2158 | CB | SER | A | 285 | 35.646 | 55.056 | 60.862 | 1.00 | 34.55 | C |
| ATOM | 2159 | OG | SER | A | 285 | 35.242 | 55.916 | 61.916 | 1.00 | 36.62 | O |
| ATOM | 2160 | C | SER | A | 285 | 37.559 | 53.978 | 59.722 | 1.00 | 37.48 | C |
| ATOM | 2161 | O | SER | A | 285 | 37.566 | 54.335 | 58.544 | 1.00 | 36.83 | O |
| ATOM | 2162 | N | ILE | A | 286 | 37.837 | 52.744 | 60.125 | 1.00 | 38.02 | N |
| ATOM | 2163 | CA | ILE | A | 286 | 38.190 | 51.678 | 59.203 | 1.00 | 37.18 | C |
| ATOM | 2164 | CB | ILE | A | 286 | 39.609 | 51.171 | 59.463 | 1.00 | 33.48 | C |
| ATOM | 2165 | CG2 | ILE | A | 286 | 40.044 | 50.259 | 58.357 | 1.00 | 25.97 | C |
| ATOM | 2166 | CG1 | ILE | A | 286 | 40.569 | 52.361 | 59.552 | 1.00 | 38.35 | C |
| ATOM | 2167 | CD1 | ILE | A | 286 | 41.926 | 52.043 | 60.195 | 1.00 | 30.83 | C |
| ATOM | 2168 | C | ILE | A | 286 | 37.215 | 50.531 | 59.380 | 1.00 | 38.33 | C |
| ATOM | 2169 | O | ILE | A | 286 | 37.167 | 49.925 | 60.440 | 1.00 | 39.09 | O |
| ATOM | 2170 | N | LYS | A | 287 | 36.425 | 50.271 | 58.343 | 1.00 | 38.04 | N |
| ATOM | 2171 | CA | LYS | A | 287 | 35.470 | 49.177 | 58.356 | 1.00 | 40.95 | C |
| ATOM | 2172 | CB | LYS | A | 287 | 34.067 | 49.665 | 57.966 | 1.00 | 37.46 | C |
| ATOM | 2173 | CG | LYS | A | 287 | 33.021 | 48.539 | 57.951 | 1.00 | 36.46 | C |
| ATOM | 2174 | CD | LYS | A | 287 | 31.688 | 49.004 | 57.474 | 1.00 | 43.46 | C |
| ATOM | 2175 | CE | LYS | A | 287 | 30.736 | 49.334 | 58.625 | 1.00 | 47.72 | C |
| ATOM | 2176 | NZ | LYS | A | 287 | 29.326 | 49.550 | 58.143 | 1.00 | 45.55 | N |
| ATOM | 2177 | C | LYS | A | 287 | 35.880 | 48.030 | 57.430 | 1.00 | 40.82 | C |
| ATOM | 2178 | O | LYS | A | 287 | 36.215 | 48.231 | 56.287 | 1.00 | 38.43 | O |
| ATOM | 2179 | N | MET | A | 288 | 35.856 | 46.815 | 57.950 | 1.00 | 38.05 | N |
| ATOM | 2180 | CA | MET | A | 288 | 36.189 | 45.632 | 57.164 | 1.00 | 41.38 | C |
| ATOM | 2181 | CB | MET | A | 288 | 37.323 | 44.856 | 57.805 | 1.00 | 38.21 | C |
| ATOM | 2182 | CG | MET | A | 288 | 37.783 | 43.659 | 56.997 | 1.00 | 32.50 | C |
| ATOM | 2183 | SD | MET | A | 288 | 38.858 | 42.551 | 57.960 | 1.00 | 43.24 | S |
| ATOM | 2184 | CE | MET | A | 288 | 40.357 | 43.689 | 58.205 | 1.00 | 27.20 | C |
| ATOM | 2185 | C | MET | A | 288 | 34.940 | 44.761 | 57.081 | 1.00 | 45.75 | C |
| ATOM | 2186 | O | MET | A | 288 | 34.464 | 44.223 | 58.082 | 1.00 | 40.81 | O |
| ATOM | 2187 | N | SER | A | 289 | 34.416 | 44.655 | 55.861 | 1.00 | 45.62 | N |
| ATOM | 2188 | CA | SER | A | 289 | 33.185 | 43.921 | 55.555 | 1.00 | 44.93 | C |
| ATOM | 2189 | CB | SER | A | 289 | 32.281 | 44.778 | 54.663 | 1.00 | 39.56 | C |
| ATOM | 2190 | OG | SER | A | 289 | 32.061 | 46.032 | 55.261 | 1.00 | 41.05 | O |
| ATOM | 2191 | C | SER | A | 289 | 33.431 | 42.603 | 54.858 | 1.00 | 45.83 | C |
| ATOM | 2192 | O | SER | A | 289 | 34.118 | 42.562 | 53.837 | 1.00 | 50.52 | O |
| ATOM | 2193 | N | ASP | A | 290 | 32.876 | 41.526 | 55.409 | 1.00 | 45.57 | N |
| ATOM | 2194 | CA | ASP | A | 290 | 33.028 | 40.202 | 54.813 | 1.00 | 44.83 | C |
| ATOM | 2195 | CB | ASP | A | 290 | 33.933 | 39.316 | 55.676 | 1.00 | 46.66 | C |
| ATOM | 2196 | CG | ASP | A | 290 | 33.245 | 38.798 | 56.950 | 1.00 | 47.08 | C |
| ATOM | 2197 | OD1 | ASP | A | 290 | 32.181 | 38.155 | 56.860 | 1.00 | 41.53 | O |
| ATOM | 2198 | OD2 | ASP | A | 290 | 33.788 | 39.024 | 58.052 | 1.00 | 45.39 | O |
| ATOM | 2199 | C | ASP | A | 290 | 31.683 | 39.508 | 54.619 | 1.00 | 50.24 | C |
| ATOM | 2200 | O | ASP | A | 290 | 30.657 | 39.983 | 55.103 | 1.00 | 49.44 | O |
| ATOM | 2201 | N | ARG | A | 291 | 31.690 | 38.389 | 53.895 | 1.00 | 51.04 | N |
| ATOM | 2202 | CA | ARG | A | 291 | 30.474 | 37.600 | 53.640 | 1.00 | 51.60 | C |
| ATOM | 2203 | CB | ARG | A | 291 | 30.090 | 37.652 | 52.158 | 1.00 | 54.18 | C |
| ATOM | 2204 | CG | ARG | A | 291 | 29.678 | 39.032 | 51.736 | 1.00 | 62.39 | C |
| ATOM | 2205 | CD | ARG | A | 291 | 29.222 | 39.119 | 50.300 | 1.00 | 72.21 | C |
| ATOM | 2206 | NE | ARG | A | 291 | 28.773 | 40.481 | 50.000 | 1.00 | 83.21 | N |
| ATOM | 2207 | CZ | ARG | A | 291 | 29.529 | 41.576 | 50.114 | 1.00 | 87.95 | C |
| ATOM | 2208 | NH1 | ARG | A | 291 | 30.793 | 41.486 | 50.517 | 1.00 | 87.63 | N |
| ATOM | 2209 | NH2 | ARG | A | 291 | 29.010 | 42.772 | 49.852 | 1.00 | 91.42 | N |
| ATOM | 2210 | C | ARG | A | 291 | 30.701 | 36.160 | 54.066 | 1.00 | 49.30 | C |
| ATOM | 2211 | O | ARG | A | 291 | 30.404 | 35.223 | 53.329 | 1.00 | 49.76 | O |
| ATOM | 2212 | N | GLY | A | 292 | 31.230 | 36.004 | 55.274 | 1.00 | 44.83 | N |
| ATOM | 2213 | CA | GLY | A | 292 | 31.539 | 34.692 | 55.805 | 1.00 | 41.19 | C |
| ATOM | 2214 | C | GLY | A | 292 | 30.430 | 34.025 | 56.583 | 1.00 | 39.48 | C |
| ATOM | 2215 | O | GLY | A | 292 | 30.704 | 33.227 | 57.481 | 1.00 | 39.70 | O |
| ATOM | 2216 | N | GLY | A | 293 | 29.186 | 34.357 | 56.249 | 1.00 | 41.80 | N |
| ATOM | 2217 | CA | GLY | A | 293 | 28.052 | 33.755 | 56.925 | 1.00 | 41.93 | C |
| ATOM | 2218 | C | GLY | A | 293 | 27.506 | 34.450 | 58.157 | 1.00 | 43.11 | C |
| ATOM | 2219 | O | GLY | A | 293 | 26.388 | 34.196 | 58.558 | 1.00 | 44.49 | O |
| ATOM | 2220 | N | GLY | A | 294 | 28.284 | 35.318 | 58.776 | 1.00 | 39.16 | N |
| ATOM | 2221 | CA | GLY | A | 294 | 27.795 | 35.968 | 59.967 | 1.00 | 43.97 | C |
| ATOM | 2222 | C | GLY | A | 294 | 27.748 | 35.091 | 61.210 | 1.00 | 48.99 | C |
| ATOM | 2223 | O | GLY | A | 294 | 27.993 | 33.883 | 61.171 | 1.00 | 50.22 | O |
| ATOM | 2224 | N | VAL | A | 295 | 27.422 | 35.734 | 62.322 | 1.00 | 46.49 | N |
| ATOM | 2225 | CA | VAL | A | 295 | 27.321 | 35.091 | 63.615 | 1.00 | 43.61 | C |
| ATOM | 2226 | CB | VAL | A | 295 | 28.549 | 35.503 | 64.487 | 1.00 | 47.59 | C |
| ATOM | 2227 | CG1 | VAL | A | 295 | 28.347 | 35.127 | 65.952 | 1.00 | 43.15 | C |
| ATOM | 2228 | CG2 | VAL | A | 295 | 29.802 | 34.860 | 63.932 | 1.00 | 47.24 | C |
| ATOM | 2229 | C | VAL | A | 295 | 26.013 | 35.557 | 64.283 | 1.00 | 44.14 | C |
| ATOM | 2230 | O | VAL | A | 295 | 25.586 | 36.688 | 64.089 | 1.00 | 43.23 | O |
| ATOM | 2231 | N | PRO | A | 296 | 25.362 | 34.682 | 65.067 | 1.00 | 42.97 | N |
| ATOM | 2232 | CD | PRO | A | 296 | 25.784 | 33.318 | 65.435 | 1.00 | 39.61 | C |
| ATOM | 2233 | CA | PRO | A | 296 | 24.113 | 35.043 | 65.756 | 1.00 | 42.84 | C |
| ATOM | 2234 | CB | PRO | A | 296 | 23.686 | 33.729 | 66.412 | 1.00 | 42.74 | C |
| ATOM | 2235 | CG | PRO | A | 296 | 24.493 | 32.670 | 65.713 | 1.00 | 38.08 | C |
| ATOM | 2236 | C | PRO | A | 296 | 24.441 | 36.108 | 66.821 | 1.00 | 46.29 | C |
| ATOM | 2237 | O | PRO | A | 296 | 25.455 | 35.985 | 67.537 | 1.00 | 43.72 | O |
| ATOM | 2238 | N | LEU | A | 297 | 23.603 | 37.141 | 66.919 | 1.00 | 44.49 | N |
| ATOM | 2239 | CA | LEU | A | 297 | 23.836 | 38.198 | 67.895 | 1.00 | 46.43 | C |
| ATOM | 2240 | CB | LEU | A | 297 | 22.617 | 39.102 | 68.027 | 1.00 | 43.89 | C |
| ATOM | 2241 | CG | LEU | A | 297 | 22.821 | 40.289 | 68.986 | 1.00 | 44.15 | C |
| ATOM | 2242 | CD1 | LEU | A | 297 | 23.967 | 41.199 | 68.483 | 1.00 | 40.17 | C |
| ATOM | 2243 | CD2 | LEU | A | 297 | 21.510 | 41.061 | 69.124 | 1.00 | 38.88 | C |
| ATOM | 2244 | C | LEU | A | 297 | 24.206 | 37.674 | 69.282 | 1.00 | 47.01 | C |
| ATOM | 2245 | O | LEU | A | 297 | 25.030 | 38.259 | 69.978 | 1.00 | 43.60 | O |
| ATOM | 2246 | N | ARG | A | 298 | 23.606 | 36.552 | 69.658 | 1.00 | 51.11 | N |
| ATOM | 2247 | CA | ARG | A | 298 | 23.839 | 35.921 | 70.951 | 1.00 | 55.06 | C |
| ATOM | 2248 | CB | ARG | A | 298 | 22.908 | 34.714 | 71.097 | 1.00 | 59.65 | C |
| ATOM | 2249 | CG | ARG | A | 298 | 23.101 | 33.932 | 72.362 | 1.00 | 65.99 | C |
| ATOM | 2250 | CD | ARG | A | 298 | 22.262 | 32.650 | 72.359 | 1.00 | 71.67 | C |
| ATOM | 2251 | NE | ARG | A | 298 | 23.092 | 31.450 | 72.196 | 1.00 | 76.11 | N |
| ATOM | 2252 | CZ | ARG | A | 298 | 23.099 | 30.687 | 71.109 | 1.00 | 77.42 | C |
| ATOM | 2253 | NH1 | ARG | A | 298 | 22.319 | 30.990 | 70.088 | 1.00 | 79.82 | N |
| ATOM | 2254 | NH2 | ARG | A | 298 | 23.889 | 29.631 | 71.040 | 1.00 | 80.11 | N |
| ATOM | 2255 | C | ARG | A | 298 | 25.280 | 35.482 | 71.210 | 1.00 | 54.34 | C |
| ATOM | 2256 | O | ARG | A | 298 | 25.743 | 35.558 | 72.339 | 1.00 | 53.31 | O |
| ATOM | 2257 | N | LYS | A | 299 | 25.976 | 35.020 | 70.174 | 1.00 | 56.52 | N |
| ATOM | 2258 | CA | LYS | A | 299 | 27.361 | 34.561 | 70.305 | 1.00 | 59.64 | C |
| ATOM | 2259 | CB | LYS | A | 299 | 27.656 | 33.463 | 69.278 | 1.00 | 62.23 | C |
| ATOM | 2260 | CG | LYS | A | 299 | 26.874 | 32.183 | 69.472 | 1.00 | 69.44 | C |
| ATOM | 2261 | CD | LYS | A | 299 | 27.175 | 31.185 | 68.364 | 1.00 | 74.21 | C |
| ATOM | 2262 | CE | LYS | A | 299 | 26.464 | 29.868 | 68.588 | 1.00 | 77.72 | C |
| ATOM | 2263 | NZ | LYS | A | 299 | 26.582 | 28.965 | 67.407 | 1.00 | 81.79 | N |
| ATOM | 2264 | C | LYS | A | 299 | 28.406 | 35.663 | 70.129 | 1.00 | 60.29 | C |
| ATOM | 2265 | O | LYS | A | 299 | 29.590 | 35.452 | 70.439 | 1.00 | 59.96 | O |
| ATOM | 2266 | N | ILE | A | 300 | 27.986 | 36.820 | 69.619 | 1.00 | 60.01 | N |
| ATOM | 2267 | CA | ILE | A | 300 | 28.915 | 37.923 | 69.394 | 1.00 | 62.04 | C |
| ATOM | 2268 | CB | ILE | A | 300 | 28.193 | 39.124 | 68.743 | 1.00 | 63.11 | C |
| ATOM | 2269 | CG2 | ILE | A | 300 | 29.042 | 40.368 | 68.880 | 1.00 | 64.45 | C |
| ATOM | 2270 | CG1 | ILE | A | 300 | 27.915 | 38.838 | 67.261 | 1.00 | 64.20 | C |
| ATOM | 2271 | CD1 | ILE | A | 300 | 29.181 | 38.681 | 66.422 | 1.00 | 65.01 | C |
| ATOM | 2272 | C | ILE | A | 300 | 29.592 | 38.364 | 70.699 | 1.00 | 61.58 | C |
| ATOM | 2273 | O | ILE | A | 300 | 30.801 | 38.568 | 70.750 | 1.00 | 56.13 | O |
| ATOM | 2274 | N | GLU | A | 301 | 28.796 | 38.497 | 71.749 | 1.00 | 63.59 | N |
| ATOM | 2275 | CA | GLU | A | 301 | 29.276 | 38.911 | 73.064 | 1.00 | 70.00 | C |
| ATOM | 2276 | CB | GLU | A | 301 | 28.132 | 38.802 | 74.078 | 1.00 | 78.54 | C |
| ATOM | 2277 | CG | GLU | A | 301 | 28.346 | 39.529 | 75.414 | 1.00 | 88.47 | C |
| ATOM | 2278 | CD | GLU | A | 301 | 28.108 | 41.038 | 75.314 | 1.00 | 95.10 | C |
| ATOM | 2279 | OE1 | GLU | A | 301 | 27.676 | 41.528 | 74.239 | 1.00 | 96.60 | O |
| ATOM | 2280 | OE2 | GLU | A | 301 | 28.350 | 41.737 | 76.319 | 1.00 | 97.57 | O |
| ATOM | 2281 | C | GLU | A | 301 | 30.443 | 38.052 | 73.540 | 1.00 | 69.25 | C |
| ATOM | 2282 | O | GLU | A | 301 | 31.407 | 38.548 | 74.116 | 1.00 | 70.45 | O |
| ATOM | 2283 | N | ARG | A | 302 | 30.336 | 36.753 | 73.299 | 1.00 | 67.01 | N |
| ATOM | 2284 | CA | ARG | A | 302 | 31.349 | 35.795 | 73.714 | 1.00 | 66.44 | C |
| ATOM | 2285 | CB | ARG | A | 302 | 30.753 | 34.387 | 73.661 | 1.00 | 71.05 | C |
| ATOM | 2286 | CG | ARG | A | 302 | 29.780 | 34.139 | 74.787 | 1.00 | 80.91 | C |
| ATOM | 2287 | CD | ARG | A | 302 | 28.843 | 32.972 | 74.551 | 1.00 | 86.33 | C |
| ATOM | 2288 | NE | ARG | A | 302 | 28.170 | 32.671 | 75.809 | 1.00 | 93.47 | N |
| ATOM | 2289 | CZ | ARG | A | 302 | 28.696 | 31.926 | 76.777 | 1.00 | 95.21 | C |
| ATOM | 2290 | NH1 | ARG | A | 302 | 29.897 | 31.390 | 76.620 | 1.00 | 94.17 | N |
| ATOM | 2291 | NH2 | ARG | A | 302 | 28.036 | 31.746 | 77.914 | 1.00 | 95.53 | N |
| ATOM | 2292 | C | ARG | A | 302 | 32.656 | 35.823 | 72.936 | 1.00 | 63.06 | C |
| ATOM | 2293 | O | ARG | A | 302 | 33.668 | 35.318 | 73.410 | 1.00 | 61.75 | O |
| ATOM | 2294 | N | LEU | A | 303 | 32.639 | 36.400 | 71.742 | 1.00 | 59.88 | N |
| ATOM | 2295 | CA | LEU | A | 303 | 33.841 | 36.466 | 70.915 | 1.00 | 55.56 | C |
| ATOM | 2296 | CB | LEU | A | 303 | 33.504 | 36.977 | 69.503 | 1.00 | 55.13 | C |
| ATOM | 2297 | CG | LEU | A | 303 | 32.477 | 36.196 | 68.676 | 1.00 | 55.04 | C |
| ATOM | 2298 | CD1 | LEU | A | 303 | 32.392 | 36.785 | 67.267 | 1.00 | 49.67 | C |
| ATOM | 2299 | CD2 | LEU | A | 303 | 32.872 | 34.728 | 68.614 | 1.00 | 52.00 | C |
| ATOM | 2300 | C | LEU | A | 303 | 34.927 | 37.350 | 71.527 | 1.00 | 54.85 | C |
| ATOM | 2301 | O | LEU | A | 303 | 36.097 | 37.205 | 71.189 | 1.00 | 55.96 | O |
| ATOM | 2302 | N | PHE | A | 304 | 34.557 | 38.265 | 72.415 | 1.00 | 50.53 | N |
| ATOM | 2303 | CA | PHE | A | 304 | 35.560 | 39.134 | 73.022 | 1.00 | 51.77 | C |
| ATOM | 2304 | CB | PHE | A | 304 | 35.050 | 40.587 | 73.105 | 1.00 | 46.31 | C |
| ATOM | 2305 | CG | PHE | A | 304 | 35.008 | 41.312 | 71.779 | 1.00 | 47.21 | C |
| ATOM | 2306 | CD1 | PHE | A | 304 | 33.872 | 41.278 | 70.975 | 1.00 | 45.69 | C |
| ATOM | 2307 | CD2 | PHE | A | 304 | 36.096 | 42.055 | 71.354 | 1.00 | 48.86 | C |
| ATOM | 2308 | CE1 | PHE | A | 304 | 33.823 | 41.974 | 69.780 | 1.00 | 45.90 | C |
| ATOM | 2309 | CE2 | PHE | A | 304 | 36.057 | 42.759 | 70.150 | 1.00 | 46.17 | C |
| ATOM | 2310 | CZ | PHE | A | 304 | 34.922 | 42.719 | 69.366 | 1.00 | 47.59 | C |
| ATOM | 2311 | C | PHE | A | 304 | 35.977 | 38.649 | 74.420 | 1.00 | 52.73 | C |
| ATOM | 2312 | O | PHE | A | 304 | 36.781 | 39.297 | 75.109 | 1.00 | 51.04 | O |
| ATOM | 2313 | N | SER | A | 305 | 35.435 | 37.502 | 74.823 | 1.00 | 54.23 | N |
| ATOM | 2314 | CA | SER | A | 305 | 35.715 | 36.924 | 76.136 | 1.00 | 56.49 | C |
| ATOM | 2315 | CB | SER | A | 305 | 34.539 | 36.034 | 76.549 | 1.00 | 58.29 | C |
| ATOM | 2316 | OG | SER | A | 305 | 34.795 | 35.391 | 77.778 | 1.00 | 60.94 | O |
| ATOM | 2317 | C | SER | A | 305 | 37.023 | 36.127 | 76.216 | 1.00 | 56.57 | C |
| ATOM | 2318 | O | SER | A | 305 | 37.325 | 35.332 | 75.335 | 1.00 | 54.50 | O |
| ATOM | 2319 | N | TYR | A | 306 | 37.789 | 36.351 | 77.281 | 1.00 | 57.79 | N |
| ATOM | 2320 | CA | TYR | A | 306 | 39.049 | 35.647 | 77.461 | 1.00 | 62.20 | C |
| ATOM | 2321 | CB | TYR | A | 306 | 39.894 | 36.316 | 78.553 | 1.00 | 56.42 | C |
| ATOM | 2322 | CG | TYR | A | 306 | 40.459 | 37.654 | 78.170 | 1.00 | 55.78 | C |
| ATOM | 2323 | CD1 | TYR | A | 306 | 41.502 | 37.752 | 77.251 | 1.00 | 49.10 | C |
| ATOM | 2324 | CE1 | TYR | A | 306 | 42.037 | 38.996 | 76.902 | 1.00 | 50.67 | C |
| ATOM | 2325 | CD2 | TYR | A | 306 | 39.958 | 38.830 | 78.736 | 1.00 | 52.43 | C |
| ATOM | 2326 | CE2 | TYR | A | 306 | 40.480 | 40.071 | 78.406 | 1.00 | 49.96 | C |
| ATOM | 2327 | CZ | TYR | A | 306 | 41.517 | 40.155 | 77.492 | 1.00 | 52.07 | C |
| ATOM | 2328 | OH | TYR | A | 306 | 42.031 | 41.403 | 77.182 | 1.00 | 50.89 | O |
| ATOM | 2329 | C | TYR | A | 306 | 38.764 | 34.203 | 77.855 | 1.00 | 67.45 | C |
| ATOM | 2330 | O | TYR | A | 306 | 39.349 | 33.264 | 77.300 | 1.00 | 65.59 | O |
| ATOM | 2331 | N | MET | A | 307 | 37.853 | 34.041 | 78.812 | 1.00 | 73.62 | N |
| ATOM | 2332 | CA | MET | A | 307 | 37.490 | 32.730 | 79.320 | 1.00 | 79.81 | C |
| ATOM | 2333 | CB | MET | A | 307 | 36.590 | 32.876 | 80.544 | 1.00 | 80.03 | C |
| ATOM | 2334 | CG | MET | A | 307 | 36.212 | 31.545 | 81.159 | 1.00 | 85.34 | C |
| ATOM | 2335 | SD | MET | A | 307 | 35.432 | 31.736 | 82.758 | 1.00 | 92.71 | S |
| ATOM | 2336 | CE | MET | A | 307 | 33.721 | 31.957 | 82.272 | 1.00 | 88.62 | C |
| ATOM | 2337 | C | MET | A | 307 | 36.802 | 31.850 | 78.292 | 1.00 | 83.74 | C |
| ATOM | 2338 | O | MET | A | 307 | 37.196 | 30.704 | 78.082 | 1.00 | 82.34 | O |
| ATOM | 2339 | N | TYR | A | 308 | 35.772 | 32.392 | 77.655 | 1.00 | 89.28 | N |
| ATOM | 2340 | CA | TYR | A | 308 | 35.009 | 31.647 | 76.667 | 1.00 | 95.13 | C |
| ATOM | 2341 | CB | TYR | A | 308 | 33.713 | 32.405 | 76.346 | 1.00 | 96.09 | C |
| ATOM | 2342 | CG | TYR | A | 308 | 32.782 | 32.552 | 77.536 | 1.00 | 97.56 | C |
| ATOM | 2343 | CD1 | TYR | A | 308 | 32.102 | 33.750 | 77.765 | 1.00 | 98.79 | C |
| ATOM | 2344 | CE1 | TYR | A | 308 | 31.252 | 33.904 | 78.861 | 1.00 | 98.34 | C |
| ATOM | 2345 | CD2 | TYR | A | 308 | 32.586 | 31.500 | 78.437 | 1.00 | 97.88 | C |
| ATOM | 2346 | CE2 | TYR | A | 308 | 31.736 | 31.642 | 79.537 | 1.00 | 98.76 | C |
| ATOM | 2347 | CZ | TYR | A | 308 | 31.072 | 32.847 | 79.746 | 1.00 | 99.58 | C |
| ATOM | 2348 | OH | TYR | A | 308 | 30.236 | 33.002 | 80.834 | 1.00 | 98.65 | O |
| ATOM | 2349 | C | TYR | A | 308 | 35.787 | 31.348 | 75.387 | 1.00 | 97.49 | C |
| ATOM | 2350 | O | TYR | A | 308 | 35.508 | 30.362 | 74.704 | 1.00 | 98.90 | O |
| ATOM | 2351 | N | SER | A | 309 | 36.765 | 32.187 | 75.067 | 1.00 | 99.83 | N |
| ATOM | 2352 | CA | SER | A | 309 | 37.556 | 31.984 | 73.858 | 1.00 | 102.43 | C |
| ATOM | 2353 | CB | SER | A | 309 | 37.885 | 33.331 | 73.214 | 1.00 | 102.43 | C |
| ATOM | 2354 | OG | SER | A | 309 | 38.775 | 34.081 | 74.025 | 1.00 | 103.08 | O |
| ATOM | 2355 | C | SER | A | 309 | 38.853 | 31.218 | 74.125 | 1.00 | 104.36 | C |
| ATOM | 2356 | O | SER | A | 309 | 39.763 | 31.226 | 73.297 | 1.00 | 105.41 | O |
| ATOM | 2357 | N | THR | A | 310 | 38.939 | 30.557 | 75.276 | 1.00 | 106.09 | N |
| ATOM | 2358 | CA | THR | A | 310 | 40.138 | 29.794 | 75.619 | 1.00 | 107.58 | C |
| ATOM | 2359 | CB | THR | A | 310 | 40.689 | 30.200 | 77.019 | 1.00 | 108.33 | C |
| ATOM | 2360 | OG1 | THR | A | 310 | 41.108 | 31.571 | 76.995 | 1.00 | 107.90 | O |
| ATOM | 2361 | CG2 | THR | A | 310 | 41.880 | 29.334 | 77.404 | 1.00 | 108.83 | C |
| ATOM | 2362 | C | THR | A | 310 | 39.869 | 28.287 | 75.602 | 1.00 | 107.05 | C |
| ATOM | 2363 | O | THR | A | 310 | 38.803 | 27.823 | 76.007 | 1.00 | 105.46 | O |
| ATOM | 2364 | N | PRO | A | 322 | 47.037 | 26.505 | 61.307 | 1.00 | 125.32 | N |
| ATOM | 2365 | CD | PRO | A | 322 | 46.848 | 26.073 | 62.705 | 1.00 | 124.43 | C |
| ATOM | 2366 | CA | PRO | A | 322 | 45.809 | 27.118 | 60.773 | 1.00 | 126.04 | C |
| ATOM | 2367 | CB | PRO | A | 322 | 44.754 | 26.666 | 61.764 | 1.00 | 125.21 | C |
| ATOM | 2368 | CG | PRO | A | 322 | 45.450 | 25.651 | 62.661 | 1.00 | 124.33 | C |
| ATOM | 2369 | C | PRO | A | 322 | 45.962 | 28.645 | 60.779 | 1.00 | 126.32 | C |
| ATOM | 2370 | O | PRO | A | 322 | 47.067 | 29.153 | 60.500 | 1.00 | 127.30 | O |
| ATOM | 2371 | N | LEU | A | 323 | 44.867 | 29.350 | 61.075 | 1.00 | 125.98 | N |
| ATOM | 2372 | CA | LEU | A | 323 | 44.902 | 30.790 | 61.152 | 1.00 | 124.60 | C |
| ATOM | 2373 | CB | LEU | A | 323 | 43.858 | 31.414 | 60.214 | 1.00 | 121.41 | C |
| ATOM | 2374 | CG | LEU | A | 323 | 44.255 | 31.774 | 58.751 | 1.00 | 118.77 | C |
| ATOM | 2375 | CD1 | LEU | A | 323 | 44.373 | 30.507 | 57.950 | 1.00 | 118.17 | C |
| ATOM | 2376 | CD2 | LEU | A | 323 | 43.196 | 32.678 | 58.151 | 1.00 | 117.35 | C |
| ATOM | 2377 | C | LEU | A | 323 | 44.577 | 31.114 | 62.604 | 1.00 | 125.26 | C |
| ATOM | 2378 | O | LEU | A | 323 | 45.265 | 31.914 | 63.266 | 1.00 | 124.98 | O |
| ATOM | 2379 | N | ALA | A | 324 | 43.477 | 30.539 | 63.077 | 1.00 | 126.38 | N |
| ATOM | 2380 | CA | ALA | A | 324 | 42.977 | 30.740 | 64.433 | 1.00 | 126.64 | C |
| ATOM | 2381 | CB | ALA | A | 324 | 41.460 | 30.914 | 64.350 | 1.00 | 126.17 | C |
| ATOM | 2382 | C | ALA | A | 324 | 43.315 | 29.619 | 65.404 | 1.00 | 126.69 | C |
| ATOM | 2383 | O | ALA | A | 324 | 43.480 | 28.461 | 65.013 | 1.00 | 126.73 | O |
| ATOM | 2384 | N | GLY | A | 325 | 43.415 | 29.994 | 66.674 | 1.00 | 126.72 | N |
| ATOM | 2385 | CA | GLY | A | 325 | 43.719 | 29.050 | 67.733 | 1.00 | 126.91 | C |
| ATOM | 2386 | C | GLY | A | 325 | 43.053 | 29.522 | 69.012 | 1.00 | 127.28 | C |
| ATOM | 2387 | O | GLY | A | 325 | 41.861 | 29.291 | 69.216 | 1.00 | 126.90 | O |
| ATOM | 2388 | N | PHE | A | 326 | 43.824 | 30.191 | 69.867 | 1.00 | 127.57 | N |
| ATOM | 2389 | CA | PHE | A | 326 | 43.326 | 30.740 | 71.131 | 1.00 | 127.18 | C |
| ATOM | 2390 | CB | PHE | A | 326 | 44.496 | 31.257 | 71.975 | 1.00 | 129.68 | C |
| ATOM | 2391 | CG | PHE | A | 326 | 45.420 | 32.174 | 71.221 | 1.00 | 132.38 | C |
| ATOM | 2392 | CD1 | PHE | A | 326 | 45.192 | 33.548 | 71.179 | 1.00 | 132.13 | C |
| ATOM | 2393 | CD2 | PHE | A | 326 | 46.501 | 31.651 | 70.515 | 1.00 | 132.55 | C |
| ATOM | 2394 | CE1 | PHE | A | 326 | 46.028 | 34.389 | 70.444 | 1.00 | 132.25 | C |
| ATOM | 2395 | CE2 | PHE | A | 326 | 47.344 | 32.476 | 69.775 | 1.00 | 132.73 | C |
| ATOM | 2396 | CZ | PHE | A | 326 | 47.107 | 33.849 | 69.740 | 1.00 | 133.37 | C |
| ATOM | 2397 | C | PHE | A | 326 | 42.350 | 31.880 | 70.851 | 1.00 | 124.62 | C |
| ATOM | 2398 | O | PHE | A | 326 | 42.088 | 32.211 | 69.696 | 1.00 | 125.34 | O |
| ATOM | 2399 | N | GLY | A | 327 | 41.823 | 32.491 | 71.904 | 1.00 | 120.69 | N |
| ATOM | 2400 | CA | GLY | A | 327 | 40.875 | 33.571 | 71.702 | 1.00 | 115.77 | C |
| ATOM | 2401 | C | GLY | A | 327 | 41.339 | 34.913 | 72.221 | 1.00 | 111.98 | C |
| ATOM | 2402 | O | GLY | A | 327 | 40.647 | 35.543 | 73.024 | 1.00 | 114.41 | O |
| ATOM | 2403 | N | TYR | A | 328 | 42.503 | 35.362 | 71.763 | 1.00 | 105.23 | N |
| ATOM | 2404 | CA | TYR | A | 328 | 43.022 | 36.645 | 72.214 | 1.00 | 96.37 | C |
| ATOM | 2405 | CB | TYR | A | 328 | 44.351 | 36.435 | 72.944 | 1.00 | 100.17 | C |
| ATOM | 2406 | CG | TYR | A | 328 | 44.171 | 35.578 | 74.182 | 1.00 | 102.95 | C |
| ATOM | 2407 | CD1 | TYR | A | 328 | 43.134 | 35.838 | 75.077 | 1.00 | 103.90 | C |
| ATOM | 2408 | CE1 | TYR | A | 328 | 42.919 | 35.037 | 76.186 | 1.00 | 105.88 | C |
| ATOM | 2409 | CD2 | TYR | A | 328 | 45.000 | 34.485 | 74.436 | 1.00 | 105.93 | C |
| ATOM | 2410 | CE2 | TYR | A | 328 | 44.793 | 33.672 | 75.552 | 1.00 | 107.08 | C |
| ATOM | 2411 | CZ | TYR | A | 328 | 43.743 | 33.957 | 76.422 | 1.00 | 107.02 | C |
| ATOM | 2412 | OH | TYR | A | 328 | 43.498 | 33.164 | 77.525 | 1.00 | 106.65 | O |
| ATOM | 2413 | C | TYR | A | 328 | 43.152 | 37.659 | 71.087 | 1.00 | 86.66 | C |
| ATOM | 2414 | O | TYR | A | 328 | 43.432 | 38.837 | 71.329 | 1.00 | 84.40 | O |
| ATOM | 2415 | N | GLY | A | 329 | 42.900 | 37.202 | 69.863 | 1.00 | 75.19 | N |
| ATOM | 2416 | CA | GLY | A | 329 | 42.975 | 38.082 | 68.714 | 1.00 | 65.95 | C |
| ATOM | 2417 | C | GLY | A | 329 | 42.040 | 39.275 | 68.843 | 1.00 | 59.98 | C |
| ATOM | 2418 | O | GLY | A | 329 | 42.477 | 40.431 | 68.741 | 1.00 | 58.64 | O |
| ATOM | 2419 | N | LEU | A | 330 | 40.760 | 39.014 | 69.096 | 1.00 | 54.77 | N |
| ATOM | 2420 | CA | LEU | A | 330 | 39.792 | 40.098 | 69.221 | 1.00 | 51.25 | C |
| ATOM | 2421 | CB | LEU | A | 330 | 38.373 | 39.546 | 69.161 | 1.00 | 45.91 | C |
| ATOM | 2422 | CG | LEU | A | 330 | 37.867 | 39.352 | 67.738 | 1.00 | 45.28 | C |
| ATOM | 2423 | CD1 | LEU | A | 330 | 36.518 | 38.624 | 67.736 | 1.00 | 35.46 | C |
| ATOM | 2424 | CD2 | LEU | A | 330 | 37.787 | 40.717 | 67.039 | 1.00 | 38.90 | C |
| ATOM | 2425 | C | LEU | A | 330 | 39.947 | 40.977 | 70.460 | 1.00 | 50.10 | C |
| ATOM | 2426 | O | LEU | A | 330 | 40.156 | 42.180 | 70.342 | 1.00 | 43.42 | O |
| ATOM | 2427 | N | PRO | A | 331 | 39.846 | 40.385 | 71.662 | 1.00 | 50.23 | N |
| ATOM | 2428 | CD | PRO | A | 331 | 39.621 | 38.960 | 71.966 | 1.00 | 50.69 | C |
| ATOM | 2429 | CA | PRO | A | 331 | 39.981 | 41.169 | 72.890 | 1.00 | 50.06 | C |
| ATOM | 2430 | CB | PRO | A | 331 | 39.827 | 40.116 | 74.000 | 1.00 | 50.90 | C |
| ATOM | 2431 | CG | PRO | A | 331 | 40.211 | 38.821 | 73.328 | 1.00 | 51.27 | C |
| ATOM | 2432 | C | PRO | A | 331 | 41.256 | 42.013 | 73.016 | 1.00 | 46.99 | C |
| ATOM | 2433 | O | PRO | A | 331 | 41.197 | 43.139 | 73.525 | 1.00 | 43.40 | O |
| ATOM | 2434 | N | ILE | A | 332 | 42.391 | 41.500 | 72.551 | 1.00 | 45.35 | N |
| ATOM | 2435 | CA | ILE | A | 332 | 43.609 | 42.272 | 72.661 | 1.00 | 43.91 | C |
| ATOM | 2436 | CB | ILE | A | 332 | 44.839 | 41.355 | 72.615 | 1.00 | 44.16 | C |
| ATOM | 2437 | CG2 | ILE | A | 332 | 46.118 | 42.160 | 72.317 | 1.00 | 38.76 | C |
| ATOM | 2438 | CG1 | ILE | A | 332 | 44.927 | 40.598 | 73.962 | 1.00 | 43.51 | C |
| ATOM | 2439 | CD1 | ILE | A | 332 | 46.248 | 39.924 | 74.226 | 1.00 | 42.69 | C |
| ATOM | 2440 | C | ILE | A | 332 | 43.673 | 43.348 | 71.591 | 1.00 | 45.62 | C |
| ATOM | 2441 | O | ILE | A | 332 | 44.261 | 44.417 | 71.816 | 1.00 | 48.69 | O |
| ATOM | 2442 | N | SER | A | 333 | 43.074 | 43.091 | 70.429 | 1.00 | 43.75 | N |
| ATOM | 2443 | CA | SER | A | 333 | 43.093 | 44.101 | 69.386 | 1.00 | 40.51 | C |
| ATOM | 2444 | CB | SER | A | 333 | 42.563 | 43.523 | 68.076 | 1.00 | 40.74 | C |
| ATOM | 2445 | OG | SER | A | 333 | 43.555 | 42.760 | 67.402 | 1.00 | 42.06 | O |
| ATOM | 2446 | C | SER | A | 333 | 42.228 | 45.277 | 69.851 | 1.00 | 38.75 | C |
| ATOM | 2447 | O | SER | A | 333 | 42.538 | 46.431 | 69.567 | 1.00 | 29.43 | O |
| ATOM | 2448 | N | ARG | A | 334 | 41.151 | 44.970 | 70.572 | 1.00 | 34.72 | N |
| ATOM | 2449 | CA | ARG | A | 334 | 40.288 | 46.010 | 71.061 | 1.00 | 38.02 | C |
| ATOM | 2450 | CB | ARG | A | 334 | 39.033 | 45.426 | 71.726 | 1.00 | 33.33 | C |
| ATOM | 2451 | CG | ARG | A | 334 | 38.030 | 46.507 | 72.080 | 1.00 | 41.36 | C |
| ATOM | 2452 | CD | ARG | A | 334 | 36.648 | 45.973 | 72.360 | 1.00 | 43.81 | C |
| ATOM | 2453 | NE | ARG | A | 334 | 36.588 | 45.167 | 73.576 | 1.00 | 49.33 | N |
| ATOM | 2454 | CZ | ARG | A | 334 | 35.505 | 44.504 | 73.980 | 1.00 | 55.10 | C |
| ATOM | 2455 | NH1 | ARG | A | 334 | 34.379 | 44.552 | 73.270 | 1.00 | 53.15 | N |
| ATOM | 2456 | NH2 | ARG | A | 334 | 35.557 | 43.768 | 75.083 | 1.00 | 51.56 | N |
| ATOM | 2457 | C | ARG | A | 334 | 41.083 | 46.880 | 72.039 | 1.00 | 38.95 | C |
| ATOM | 2458 | O | ARG | A | 334 | 41.006 | 48.117 | 71.974 | 1.00 | 34.67 | O |
| ATOM | 2459 | N | LEU | A | 335 | 41.854 | 46.246 | 72.924 | 1.00 | 38.48 | N |
| ATOM | 2460 | CA | LEU | A | 335 | 42.642 | 47.012 | 73.869 | 1.00 | 37.76 | C |
| ATOM | 2461 | CB | LEU | A | 335 | 43.427 | 46.078 | 74.797 | 1.00 | 36.61 | C |
| ATOM | 2462 | CG | LEU | A | 335 | 42.597 | 45.110 | 75.689 | 1.00 | 39.83 | C |
| ATOM | 2463 | CD1 | LEU | A | 335 | 43.522 | 44.302 | 76.587 | 1.00 | 35.10 | C |
| ATOM | 2464 | CD2 | LEU | A | 335 | 41.616 | 45.874 | 76.566 | 1.00 | 35.51 | C |
| ATOM | 2465 | C | LEU | A | 335 | 43.577 | 47.991 | 73.117 | 1.00 | 35.70 | C |
| ATOM | 2466 | O | LEU | A | 335 | 43.705 | 49.151 | 73.505 | 1.00 | 33.38 | O |
| ATOM | 2467 | N | TYR | A | 336 | 44.209 | 47.545 | 72.034 | 1.00 | 31.74 | N |
| ATOM | 2468 | CA | TYR | A | 336 | 45.086 | 48.431 | 71.269 | 1.00 | 37.74 | C |
| ATOM | 2469 | CB | TYR | A | 336 | 45.701 | 47.726 | 70.058 | 1.00 | 39.44 | C |
| ATOM | 2470 | CG | TYR | A | 336 | 46.933 | 46.921 | 70.363 | 1.00 | 46.47 | C |
| ATOM | 2471 | CD1 | TYR | A | 336 | 46.872 | 45.531 | 70.466 | 1.00 | 46.72 | C |
| ATOM | 2472 | CE1 | TYR | A | 336 | 48.010 | 44.791 | 70.774 | 1.00 | 50.09 | C |
| ATOM | 2473 | CD2 | TYR | A | 336 | 48.167 | 47.555 | 70.574 | 1.00 | 46.51 | C |
| ATOM | 2474 | CE2 | TYR | A | 336 | 49.306 | 46.835 | 70.881 | 1.00 | 48.04 | C |
| ATOM | 2475 | CZ | TYR | A | 336 | 49.226 | 45.451 | 70.982 | 1.00 | 53.54 | C |
| ATOM | 2476 | OH | TYR | A | 336 | 50.349 | 44.731 | 71.294 | 1.00 | 50.84 | O |
| ATOM | 2477 | C | TYR | A | 336 | 44.329 | 49.665 | 70.770 | 1.00 | 40.35 | C |
| ATOM | 2478 | O | TYR | A | 336 | 44.863 | 50.784 | 70.822 | 1.00 | 40.77 | O |
| ATOM | 2479 | N | ALA | A | 337 | 43.103 | 49.465 | 70.287 | 1.00 | 34.39 | N |
| ATOM | 2480 | CA | ALA | A | 337 | 42.302 | 50.567 | 69.805 | 1.00 | 36.75 | C |
| ATOM | 2481 | CB | ALA | A | 337 | 41.025 | 50.036 | 69.148 | 1.00 | 34.39 | C |
| ATOM | 2482 | C | ALA | A | 337 | 41.953 | 51.524 | 70.936 | 1.00 | 36.59 | C |
| ATOM | 2483 | O | ALA | A | 337 | 42.049 | 52.743 | 70.782 | 1.00 | 31.10 | O |
| ATOM | 2484 | N | LYS | A | 338 | 41.556 | 50.955 | 72.067 | 1.00 | 38.60 | N |
| ATOM | 2485 | CA | LYS | A | 338 | 41.174 | 51.752 | 73.221 | 1.00 | 42.30 | C |
| ATOM | 2486 | CB | LYS | A | 338 | 40.558 | 50.851 | 74.295 | 1.00 | 39.17 | C |
| ATOM | 2487 | CG | LYS | A | 338 | 39.075 | 50.661 | 74.097 | 1.00 | 39.88 | C |
| ATOM | 2488 | CD | LYS | A | 338 | 38.476 | 49.714 | 75.100 | 1.00 | 44.08 | C |
| ATOM | 2489 | CE | LYS | A | 338 | 36.968 | 49.643 | 74.916 | 1.00 | 51.30 | C |
| ATOM | 2490 | NZ | LYS | A | 338 | 36.334 | 48.492 | 75.637 | 1.00 | 56.77 | N |
| ATOM | 2491 | C | LYS | A | 338 | 42.343 | 52.545 | 73.806 | 1.00 | 43.01 | C |
| ATOM | 2492 | O | LYS | A | 338 | 42.169 | 53.629 | 74.365 | 1.00 | 41.05 | O |
| ATOM | 2493 | N | TYR | A | 339 | 43.543 | 52.001 | 73.641 | 1.00 | 40.34 | N |
| ATOM | 2494 | CA | TYR | A | 339 | 44.770 | 52.596 | 74.175 | 1.00 | 42.95 | C |
| ATOM | 2495 | CB | TYR | A | 339 | 46.003 | 51.832 | 73.647 | 1.00 | 41.44 | C |
| ATOM | 2496 | CG | TYR | A | 339 | 47.324 | 52.345 | 74.165 | 1.00 | 39.72 | C |
| ATOM | 2497 | CD1 | TYR | A | 339 | 47.494 | 52.657 | 75.524 | 1.00 | 40.60 | C |
| ATOM | 2498 | CE1 | TYR | A | 339 | 48.720 | 53.166 | 76.009 | 1.00 | 38.14 | C |
| ATOM | 2499 | CD2 | TYR | A | 339 | 48.405 | 52.538 | 73.295 | 1.00 | 42.36 | C |
| ATOM | 2500 | CE2 | TYR | A | 339 | 49.630 | 53.032 | 73.751 | 1.00 | 42.49 | C |
| ATOM | 2501 | CZ | TYR | A | 339 | 49.792 | 53.356 | 75.106 | 1.00 | 44.91 | C |
| ATOM | 2502 | OH | TYR | A | 339 | 50.993 | 53.908 | 75.541 | 1.00 | 46.18 | O |
| ATOM | 2503 | C | TYR | A | 339 | 44.915 | 54.098 | 73.909 | 1.00 | 44.41 | C |
| ATOM | 2504 | O | TYR | A | 339 | 45.336 | 54.843 | 74.784 | 1.00 | 41.93 | O |
| ATOM | 2505 | N | PHE | A | 340 | 44.586 | 54.532 | 72.696 | 1.00 | 44.39 | N |
| ATOM | 2506 | CA | PHE | A | 340 | 44.635 | 55.947 | 72.337 | 1.00 | 43.65 | C |
| ATOM | 2507 | CB | PHE | A | 340 | 45.499 | 56.154 | 71.088 | 1.00 | 44.88 | C |
| ATOM | 2508 | CG | PHE | A | 340 | 46.967 | 56.244 | 71.391 | 1.00 | 49.03 | C |
| ATOM | 2509 | CD1 | PHE | A | 340 | 47.481 | 57.341 | 72.096 | 1.00 | 49.22 | C |
| ATOM | 2510 | CD2 | PHE | A | 340 | 47.835 | 55.231 | 70.998 | 1.00 | 47.15 | C |
| ATOM | 2511 | CE1 | PHE | A | 340 | 48.848 | 57.426 | 72.403 | 1.00 | 48.26 | C |
| ATOM | 2512 | CE2 | PHE | A | 340 | 49.185 | 55.299 | 71.292 | 1.00 | 45.11 | C |
| ATOM | 2513 | CZ | PHE | A | 340 | 49.702 | 56.408 | 72.002 | 1.00 | 47.13 | C |
| ATOM | 2514 | C | PHE | A | 340 | 43.247 | 56.574 | 72.134 | 1.00 | 43.17 | C |
| ATOM | 2515 | O | PHE | A | 340 | 43.029 | 57.381 | 71.229 | 1.00 | 42.38 | O |
| ATOM | 2516 | N | GLN | A | 341 | 42.310 | 56.174 | 72.985 | 1.00 | 41.69 | N |
| ATOM | 2517 | CA | GLN | A | 341 | 40.941 | 56.675 | 72.969 | 1.00 | 41.38 | C |
| ATOM | 2518 | CB | GLN | A | 341 | 40.953 | 58.212 | 72.944 | 1.00 | 43.13 | C |
| ATOM | 2519 | CG | GLN | A | 341 | 41.712 | 58.870 | 74.074 | 1.00 | 38.43 | C |
| ATOM | 2520 | CD | GLN | A | 341 | 41.253 | 58.406 | 75.450 | 1.00 | 45.24 | C |
| ATOM | 2521 | OE1 | GLN | A | 341 | 40.051 | 58.207 | 75.686 | 1.00 | 48.58 | O |
| ATOM | 2522 | NE2 | GLN | A | 341 | 42.215 | 58.238 | 76.375 | 1.00 | 33.95 | N |
| ATOM | 2523 | C | GLN | A | 341 | 40.039 | 56.157 | 71.829 | 1.00 | 41.78 | C |
| ATOM | 2524 | O | GLN | A | 341 | 39.013 | 56.777 | 71.522 | 1.00 | 44.24 | O |
| ATOM | 2525 | N | GLY | A | 342 | 40.427 | 55.054 | 71.189 | 1.00 | 40.82 | N |
| ATOM | 2526 | CA | GLY | A | 342 | 39.620 | 54.506 | 70.117 | 1.00 | 38.42 | C |
| ATOM | 2527 | C | GLY | A | 342 | 38.779 | 53.341 | 70.583 | 1.00 | 41.09 | C |
| ATOM | 2528 | O | GLY | A | 342 | 38.571 | 53.184 | 71.770 | 1.00 | 42.52 | O |
| ATOM | 2529 | N | ASP | A | 343 | 38.309 | 52.521 | 69.645 | 1.00 | 42.61 | N |
| ATOM | 2530 | CA | ASP | A | 343 | 37.485 | 51.340 | 69.949 | 1.00 | 40.80 | C |
| ATOM | 2531 | CB | ASP | A | 343 | 36.046 | 51.770 | 70.322 | 1.00 | 32.14 | C |
| ATOM | 2532 | CG | ASP | A | 343 | 35.277 | 50.685 | 71.117 | 1.00 | 42.95 | C |
| ATOM | 2533 | OD1 | ASP | A | 343 | 35.844 | 49.603 | 71.414 | 1.00 | 40.98 | O |
| ATOM | 2534 | OD2 | ASP | A | 343 | 34.090 | 50.928 | 71.447 | 1.00 | 44.08 | O |
| ATOM | 2535 | C | ASP | A | 343 | 37.471 | 50.332 | 68.769 | 1.00 | 38.63 | C |
| ATOM | 2536 | O | ASP | A | 343 | 37.970 | 50.615 | 67.693 | 1.00 | 35.25 | O |
| ATOM | 2537 | N | LEU | A | 344 | 36.932 | 49.143 | 68.999 | 1.00 | 34.15 | N |
| ATOM | 2538 | CA | LEU | A | 344 | 36.843 | 48.121 | 67.975 | 1.00 | 35.72 | C |
| ATOM | 2539 | CB | LEU | A | 344 | 38.008 | 47.152 | 68.093 | 1.00 | 36.90 | C |
| ATOM | 2540 | CG | LEU | A | 344 | 38.257 | 46.206 | 66.915 | 1.00 | 37.88 | C |
| ATOM | 2541 | CD1 | LEU | A | 344 | 39.632 | 45.537 | 67.066 | 1.00 | 37.92 | C |
| ATOM | 2542 | CD2 | LEU | A | 344 | 37.143 | 45.199 | 66.809 | 1.00 | 32.23 | C |
| ATOM | 2543 | C | LEU | A | 344 | 35.529 | 47.384 | 68.222 | 1.00 | 38.49 | C |
| ATOM | 2544 | O | LEU | A | 344 | 35.330 | 46.785 | 69.275 | 1.00 | 37.41 | O |
| ATOM | 2545 | N | GLN | A | 345 | 34.634 | 47.420 | 67.241 | 1.00 | 36.04 | N |
| ATOM | 2546 | CA | GLN | A | 345 | 33.342 | 46.778 | 67.388 | 1.00 | 35.31 | C |
| ATOM | 2547 | CB | GLN | A | 345 | 32.236 | 47.835 | 67.526 | 1.00 | 36.03 | C |
| ATOM | 2548 | CG | GLN | A | 345 | 32.523 | 48.883 | 68.578 | 1.00 | 38.95 | C |
| ATOM | 2549 | CD | GLN | A | 345 | 31.458 | 49.937 | 68.710 | 1.00 | 44.14 | C |
| ATOM | 2550 | OE1 | GLN | A | 345 | 30.722 | 50.224 | 67.775 | 1.00 | 47.66 | O |
| ATOM | 2551 | NE2 | GLN | A | 345 | 31.390 | 50.545 | 69.880 | 1.00 | 46.93 | N |
| ATOM | 2552 | C | GLN | A | 345 | 33.004 | 45.827 | 66.256 | 1.00 | 36.09 | C |
| ATOM | 2553 | O | GLN | A | 345 | 33.520 | 45.916 | 65.157 | 1.00 | 36.60 | O |
| ATOM | 2554 | N | LEU | A | 346 | 32.118 | 44.901 | 66.574 | 1.00 | 34.00 | N |
| ATOM | 2555 | CA | LEU | A | 346 | 31.649 | 43.893 | 65.658 | 1.00 | 37.07 | C |
| ATOM | 2556 | CB | LEU | A | 346 | 32.178 | 42.519 | 66.090 | 1.00 | 38.52 | C |
| ATOM | 2557 | CG | LEU | A | 346 | 33.175 | 41.728 | 65.231 | 1.00 | 47.97 | C |
| ATOM | 2558 | CD1 | LEU | A | 346 | 34.550 | 42.407 | 65.211 | 1.00 | 48.07 | C |
| ATOM | 2559 | CD2 | LEU | A | 346 | 33.306 | 40.334 | 65.828 | 1.00 | 48.91 | C |
| ATOM | 2560 | C | LEU | A | 346 | 30.129 | 43.825 | 65.597 | 1.00 | 37.34 | C |
| ATOM | 2561 | O | LEU | A | 346 | 29.472 | 43.741 | 66.619 | 1.00 | 35.52 | O |
| ATOM | 2562 | N | PHE | A | 347 | 29.591 | 43.854 | 64.382 | 1.00 | 37.55 | N |
| ATOM | 2563 | CA | PHE | A | 347 | 28.155 | 43.702 | 64.140 | 1.00 | 42.47 | C |
| ATOM | 2564 | CB | PHE | A | 347 | 27.427 | 45.037 | 63.914 | 1.00 | 43.18 | C |
| ATOM | 2565 | CG | PHE | A | 347 | 28.329 | 46.182 | 63.772 | 1.00 | 48.59 | C |
| ATOM | 2566 | CD1 | PHE | A | 347 | 28.722 | 46.625 | 62.507 | 1.00 | 58.80 | C |
| ATOM | 2567 | CD2 | PHE | A | 347 | 28.835 | 46.804 | 64.910 | 1.00 | 60.17 | C |
| ATOM | 2568 | CE1 | PHE | A | 347 | 29.620 | 47.679 | 62.378 | 1.00 | 65.58 | C |
| ATOM | 2569 | CE2 | PHE | A | 347 | 29.737 | 47.861 | 64.808 | 1.00 | 64.65 | C |
| ATOM | 2570 | CZ | PHE | A | 347 | 30.137 | 48.306 | 63.542 | 1.00 | 66.87 | C |
| ATOM | 2571 | C | PHE | A | 347 | 28.037 | 42.803 | 62.933 | 1.00 | 42.28 | C |
| ATOM | 2572 | O | PHE | A | 347 | 28.629 | 43.038 | 61.900 | 1.00 | 38.30 | O |
| ATOM | 2573 | N | SER | A | 348 | 27.257 | 41.750 | 63.104 | 1.00 | 39.86 | N |
| ATOM | 2574 | CA | SER | A | 348 | 27.079 | 40.723 | 62.090 | 1.00 | 41.43 | C |
| ATOM | 2575 | CB | SER | A | 348 | 27.630 | 39.398 | 62.683 | 1.00 | 30.16 | C |
| ATOM | 2576 | OG | SER | A | 348 | 27.622 | 38.342 | 61.748 | 1.00 | 37.31 | O |
| ATOM | 2577 | C | SER | A | 348 | 25.608 | 40.580 | 61.634 | 1.00 | 42.37 | C |
| ATOM | 2578 | O | SER | A | 348 | 24.703 | 41.138 | 62.242 | 1.00 | 36.80 | O |
| ATOM | 2579 | N | MET | A | 349 | 25.416 | 39.825 | 60.555 | 1.00 | 41.24 | N |
| ATOM | 2580 | CA | MET | A | 349 | 24.120 | 39.518 | 59.969 | 1.00 | 43.07 | C |
| ATOM | 2581 | CB | MET | A | 349 | 23.982 | 40.173 | 58.598 | 1.00 | 46.85 | C |
| ATOM | 2582 | CG | MET | A | 349 | 24.127 | 41.666 | 58.552 | 1.00 | 50.45 | C |
| ATOM | 2583 | SD | MET | A | 349 | 22.717 | 42.519 | 59.168 | 1.00 | 58.76 | S |
| ATOM | 2584 | CE | MET | A | 349 | 21.693 | 42.677 | 57.699 | 1.00 | 52.84 | C |
| ATOM | 2585 | C | MET | A | 349 | 24.099 | 37.997 | 59.770 | 1.00 | 45.90 | C |
| ATOM | 2586 | O | MET | A | 349 | 24.503 | 37.508 | 58.718 | 1.00 | 41.65 | O |
| ATOM | 2587 | N | GLU | A | 350 | 23.636 | 37.237 | 60.757 | 1.00 | 46.42 | N |
| ATOM | 2588 | CA | GLU | A | 350 | 23.632 | 35.793 | 60.579 | 1.00 | 47.35 | C |
| ATOM | 2589 | CB | GLU | A | 350 | 22.883 | 35.083 | 61.697 | 1.00 | 52.06 | C |
| ATOM | 2590 | CG | GLU | A | 350 | 23.149 | 33.589 | 61.698 | 1.00 | 51.28 | C |
| ATOM | 2591 | CD | GLU | A | 350 | 22.188 | 32.802 | 62.572 | 1.00 | 53.23 | C |
| ATOM | 2592 | OE1 | GLU | A | 350 | 22.547 | 31.668 | 62.972 | 1.00 | 58.36 | O |
| ATOM | 2593 | OE2 | GLU | A | 350 | 21.075 | 33.302 | 62.854 | 1.00 | 52.62 | O |
| ATOM | 2594 | C | GLU | A | 350 | 23.010 | 35.415 | 59.237 | 1.00 | 48.14 | C |
| ATOM | 2595 | O | GLU | A | 350 | 21.939 | 35.896 | 58.888 | 1.00 | 46.32 | O |
| ATOM | 2596 | N | GLY | A | 351 | 23.724 | 34.577 | 58.485 | 1.00 | 48.70 | N |
| ATOM | 2597 | CA | GLY | A | 351 | 23.269 | 34.132 | 57.182 | 1.00 | 46.64 | C |
| ATOM | 2598 | C | GLY | A | 351 | 23.823 | 34.960 | 56.039 | 1.00 | 47.20 | C |
| ATOM | 2599 | O | GLY | A | 351 | 23.458 | 34.755 | 54.882 | 1.00 | 49.23 | O |
| ATOM | 2600 | N | PHE | A | 352 | 24.701 | 35.905 | 56.353 | 1.00 | 47.94 | N |
| ATOM | 2601 | CA | PHE | A | 352 | 25.271 | 36.775 | 55.327 | 1.00 | 49.02 | C |
| ATOM | 2602 | CB | PHE | A | 352 | 24.435 | 38.052 | 55.195 | 1.00 | 45.43 | C |
| ATOM | 2603 | CG | PHE | A | 352 | 24.955 | 39.004 | 54.164 | 1.00 | 47.27 | C |
| ATOM | 2604 | CD1 | PHE | A | 352 | 24.855 | 38.700 | 52.813 | 1.00 | 50.13 | C |
| ATOM | 2605 | CD2 | PHE | A | 352 | 25.564 | 40.199 | 54.532 | 1.00 | 49.89 | C |
| ATOM | 2606 | CE1 | PHE | A | 352 | 25.351 | 39.567 | 51.841 | 1.00 | 49.59 | C |
| ATOM | 2607 | CE2 | PHE | A | 352 | 26.063 | 41.071 | 53.559 | 1.00 | 51.08 | C |
| ATOM | 2608 | CZ | PHE | A | 352 | 25.952 | 40.748 | 52.215 | 1.00 | 47.91 | C |
| ATOM | 2609 | C | PHE | A | 352 | 26.733 | 37.165 | 55.536 | 1.00 | 49.52 | C |
| ATOM | 2610 | O | PHE | A | 352 | 27.584 | 36.801 | 54.743 | 1.00 | 49.04 | O |
| ATOM | 2611 | N | GLY | A | 353 | 27.007 | 37.911 | 56.600 | 1.00 | 49.02 | N |
| ATOM | 2612 | CA | GLY | A | 353 | 28.362 | 38.350 | 56.863 | 1.00 | 48.25 | C |
| ATOM | 2613 | C | GLY | A | 353 | 28.544 | 39.228 | 58.085 | 1.00 | 47.78 | C |
| ATOM | 2614 | O | GLY | A | 353 | 27.610 | 39.437 | 58.856 | 1.00 | 47.53 | O |
| ATOM | 2615 | N | THR | A | 354 | 29.750 | 39.775 | 58.239 | 1.00 | 43.01 | N |
| ATOM | 2616 | CA | THR | A | 354 | 30.090 | 40.585 | 59.400 | 1.00 | 34.65 | C |
| ATOM | 2617 | CB | THR | A | 354 | 30.915 | 39.730 | 60.414 | 1.00 | 32.06 | C |
| ATOM | 2618 | OG1 | THR | A | 354 | 30.187 | 38.541 | 60.745 | 1.00 | 25.97 | O |
| ATOM | 2619 | CG2 | THR | A | 354 | 31.198 | 40.529 | 61.712 | 1.00 | 26.10 | C |
| ATOM | 2620 | C | THR | A | 354 | 30.874 | 41.873 | 59.117 | 1.00 | 40.01 | C |
| ATOM | 2621 | O | THR | A | 354 | 31.716 | 41.916 | 58.226 | 1.00 | 34.85 | O |
| ATOM | 2622 | N | ASP | A | 355 | 30.582 | 42.916 | 59.890 | 1.00 | 36.90 | N |
| ATOM | 2623 | CA | ASP | A | 355 | 31.292 | 44.188 | 59.787 | 1.00 | 39.75 | C |
| ATOM | 2624 | CB | ASP | A | 355 | 30.320 | 45.360 | 59.638 | 1.00 | 39.56 | C |
| ATOM | 2625 | CG | ASP | A | 355 | 29.795 | 45.507 | 58.222 | 1.00 | 46.10 | C |
| ATOM | 2626 | OD1 | ASP | A | 355 | 29.920 | 44.537 | 57.439 | 1.00 | 43.06 | O |
| ATOM | 2627 | OD2 | ASP | A | 355 | 29.248 | 46.592 | 57.889 | 1.00 | 45.08 | O |
| ATOM | 2628 | C | ASP | A | 355 | 32.138 | 44.386 | 61.054 | 1.00 | 40.25 | C |
| ATOM | 2629 | O | ASP | A | 355 | 31.639 | 44.259 | 62.184 | 1.00 | 38.50 | O |
| ATOM | 2630 | N | ALA | A | 356 | 33.427 | 44.643 | 60.861 | 1.00 | 37.62 | N |
| ATOM | 2631 | CA | ALA | A | 356 | 34.315 | 44.878 | 61.977 | 1.00 | 36.64 | C |
| ATOM | 2632 | CB | ALA | A | 356 | 35.423 | 43.833 | 62.001 | 1.00 | 32.81 | C |
| ATOM | 2633 | C | ALA | A | 356 | 34.868 | 46.282 | 61.800 | 1.00 | 37.69 | C |
| ATOM | 2634 | O | ALA | A | 356 | 35.459 | 46.601 | 60.778 | 1.00 | 38.51 | O |
| ATOM | 2635 | N | VAL | A | 357 | 34.655 | 47.125 | 62.801 | 1.00 | 38.99 | N |
| ATOM | 2636 | CA | VAL | A | 357 | 35.099 | 48.504 | 62.721 | 1.00 | 36.06 | C |
| ATOM | 2637 | CB | VAL | A | 357 | 33.881 | 49.462 | 62.784 | 1.00 | 39.92 | C |
| ATOM | 2638 | CG1 | VAL | A | 357 | 34.328 | 50.895 | 62.588 | 1.00 | 37.87 | C |
| ATOM | 2639 | CG2 | VAL | A | 357 | 32.852 | 49.041 | 61.761 | 1.00 | 41.99 | C |
| ATOM | 2640 | C | VAL | A | 357 | 36.105 | 48.960 | 63.764 | 1.00 | 36.43 | C |
| ATOM | 2641 | O | VAL | A | 357 | 35.918 | 48.779 | 64.965 | 1.00 | 33.96 | O |
| ATOM | 2642 | N | ILE | A | 358 | 37.160 | 49.590 | 63.273 | 1.00 | 32.36 | N |
| ATOM | 2643 | CA | ILE | A | 358 | 38.178 | 50.147 | 64.118 | 1.00 | 34.00 | C |
| ATOM | 2644 | CB | ILE | A | 358 | 39.608 | 49.886 | 63.558 | 1.00 | 35.92 | C |
| ATOM | 2645 | CG2 | ILE | A | 358 | 40.671 | 50.490 | 64.528 | 1.00 | 31.94 | C |
| ATOM | 2646 | CG1 | ILE | A | 358 | 39.828 | 48.378 | 63.388 | 1.00 | 32.60 | C |
| ATOM | 2647 | CD1 | ILE | A | 358 | 41.146 | 48.033 | 62.719 | 1.00 | 33.90 | C |
| ATOM | 2648 | C | ILE | A | 358 | 37.959 | 51.647 | 64.175 | 1.00 | 34.55 | C |
| ATOM | 2649 | O | ILE | A | 358 | 37.986 | 52.313 | 63.156 | 1.00 | 33.78 | O |
| ATOM | 2650 | N | TYR | A | 359 | 37.728 | 52.167 | 65.372 | 1.00 | 36.84 | N |
| ATOM | 2651 | CA | TYR | A | 359 | 37.549 | 53.601 | 65.581 | 1.00 | 37.77 | C |
| ATOM | 2652 | CB | TYR | A | 359 | 36.345 | 53.862 | 66.501 | 1.00 | 36.83 | C |
| ATOM | 2653 | CG | TYR | A | 359 | 35.020 | 53.454 | 65.918 | 1.00 | 39.29 | C |
| ATOM | 2654 | CD1 | TYR | A | 359 | 34.359 | 52.310 | 66.383 | 1.00 | 35.14 | C |
| ATOM | 2655 | CE1 | TYR | A | 359 | 33.132 | 51.926 | 65.871 | 1.00 | 36.00 | C |
| ATOM | 2656 | CD2 | TYR | A | 359 | 34.423 | 54.212 | 64.905 | 1.00 | 36.43 | C |
| ATOM | 2657 | CE2 | TYR | A | 359 | 33.184 | 53.835 | 64.359 | 1.00 | 39.03 | C |
| ATOM | 2658 | CZ | TYR | A | 359 | 32.535 | 52.688 | 64.850 | 1.00 | 39.24 | C |
| ATOM | 2659 | OH | TYR | A | 359 | 31.306 | 52.314 | 64.346 | 1.00 | 35.87 | O |
| ATOM | 2660 | C | TYR | A | 359 | 38.803 | 54.243 | 66.181 | 1.00 | 40.27 | C |
| ATOM | 2661 | O | TYR | A | 359 | 39.235 | 53.886 | 67.266 | 1.00 | 41.32 | O |
| ATOM | 2662 | N | LEU | A | 360 | 39.385 | 55.184 | 65.448 | 1.00 | 41.06 | N |
| ATOM | 2663 | CA | LEU | A | 360 | 40.567 | 55.901 | 65.895 | 1.00 | 40.71 | C |
| ATOM | 2664 | CB | LEU | A | 360 | 41.734 | 55.704 | 64.931 | 1.00 | 35.86 | C |
| ATOM | 2665 | CG | LEU | A | 360 | 42.180 | 54.276 | 64.655 | 1.00 | 38.47 | C |
| ATOM | 2666 | CD1 | LEU | A | 360 | 43.278 | 54.309 | 63.603 | 1.00 | 33.08 | C |
| ATOM | 2667 | CD2 | LEU | A | 360 | 42.672 | 53.585 | 65.962 | 1.00 | 29.00 | C |
| ATOM | 2668 | C | LEU | A | 360 | 40.259 | 57.375 | 65.966 | 1.00 | 44.72 | C |
| ATOM | 2669 | O | LEU | A | 360 | 39.314 | 57.874 | 65.360 | 1.00 | 40.35 | O |
| ATOM | 2670 | N | LYS | A | 361 | 41.073 | 58.086 | 66.723 | 1.00 | 43.76 | N |
| ATOM | 2671 | CA | LYS | A | 361 | 40.878 | 59.515 | 66.828 | 1.00 | 44.09 | C |
| ATOM | 2672 | CB | LYS | A | 361 | 41.199 | 60.004 | 68.243 | 1.00 | 42.01 | C |
| ATOM | 2673 | CG | LYS | A | 361 | 39.966 | 60.663 | 68.811 | 1.00 | 45.07 | C |
| ATOM | 2674 | CD | LYS | A | 361 | 39.612 | 60.264 | 70.214 | 1.00 | 47.34 | C |
| ATOM | 2675 | CE | LYS | A | 361 | 38.197 | 60.598 | 70.443 | 1.00 | 44.52 | C |
| ATOM | 2676 | NZ | LYS | A | 361 | 37.857 | 60.958 | 71.843 | 1.00 | 57.36 | N |
| ATOM | 2677 | C | LYS | A | 361 | 41.728 | 60.201 | 65.763 | 1.00 | 45.05 | C |
| ATOM | 2678 | O | LYS | A | 361 | 42.939 | 59.981 | 65.676 | 1.00 | 47.18 | O |
| ATOM | 2679 | N | ALA | A | 362 | 41.060 | 60.985 | 64.918 | 1.00 | 44.96 | N |
| ATOM | 2680 | CA | ALA | A | 362 | 41.696 | 61.691 | 63.810 | 1.00 | 46.52 | C |
| ATOM | 2681 | CB | ALA | A | 362 | 40.625 | 62.392 | 62.995 | 1.00 | 47.04 | C |
| ATOM | 2682 | C | ALA | A | 362 | 42.804 | 62.684 | 64.179 | 1.00 | 46.09 | C |
| ATOM | 2683 | O | ALA | A | 362 | 43.847 | 62.708 | 63.545 | 1.00 | 44.40 | O |
| ATOM | 2684 | N | LEU | A | 363 | 42.570 | 63.499 | 65.202 | 1.00 | 44.40 | N |
| ATOM | 2685 | CA | LEU | A | 363 | 43.529 | 64.510 | 65.631 | 1.00 | 48.86 | C |
| ATOM | 2686 | CB | LEU | A | 363 | 42.769 | 65.731 | 66.148 | 1.00 | 51.41 | C |
| ATOM | 2687 | CG | LEU | A | 363 | 41.632 | 66.161 | 65.209 | 1.00 | 56.29 | C |
| ATOM | 2688 | CD1 | LEU | A | 363 | 41.009 | 67.451 | 65.704 | 1.00 | 52.03 | C |
| ATOM | 2689 | CD2 | LEU | A | 363 | 42.175 | 66.335 | 63.794 | 1.00 | 55.98 | C |
| ATOM | 2690 | C | LEU | A | 363 | 44.524 | 64.058 | 66.693 | 1.00 | 49.90 | C |
| ATOM | 2691 | O | LEU | A | 363 | 44.130 | 63.488 | 67.719 | 1.00 | 48.38 | O |
| ATOM | 2692 | N | SER | A | 364 | 45.802 | 64.353 | 66.461 | 1.00 | 50.70 | N |
| ATOM | 2693 | CA | SER | A | 364 | 46.859 | 63.983 | 67.394 | 1.00 | 53.12 | C |
| ATOM | 2694 | CB | SER | A | 364 | 48.215 | 64.469 | 66.877 | 1.00 | 53.73 | C |
| ATOM | 2695 | OG | SER | A | 364 | 48.311 | 65.886 | 66.899 | 1.00 | 57.92 | O |
| ATOM | 2696 | C | SER | A | 364 | 46.593 | 64.562 | 68.782 | 1.00 | 52.70 | C |
| ATOM | 2697 | O | SER | A | 364 | 47.091 | 64.062 | 69.784 | 1.00 | 53.59 | O |
| ATOM | 2698 | N | THR | A | 365 | 45.775 | 65.603 | 68.834 | 1.00 | 55.54 | N |
| ATOM | 2699 | CA | THR | A | 365 | 45.439 | 66.252 | 70.088 | 1.00 | 58.19 | C |
| ATOM | 2700 | CB | THR | A | 365 | 44.704 | 67.569 | 69.846 | 1.00 | 61.84 | C |
| ATOM | 2701 | OG1 | THR | A | 365 | 44.979 | 68.040 | 68.523 | 1.00 | 69.77 | O |
| ATOM | 2702 | CG2 | THR | A | 365 | 45.164 | 68.615 | 70.839 | 1.00 | 64.80 | C |
| ATOM | 2703 | C | THR | A | 365 | 44.541 | 65.371 | 70.960 | 1.00 | 59.19 | C |
| ATOM | 2704 | O | THR | A | 365 | 44.698 | 65.342 | 72.188 | 1.00 | 62.31 | O |
| ATOM | 2705 | N | ASP | A | 366 | 43.606 | 64.658 | 70.331 | 1.00 | 59.49 | N |
| ATOM | 2706 | CA | ASP | A | 366 | 42.682 | 63.783 | 71.055 | 1.00 | 58.19 | C |
| ATOM | 2707 | CB | ASP | A | 366 | 41.404 | 63.570 | 70.252 | 1.00 | 59.48 | C |
| ATOM | 2708 | CG | ASP | A | 366 | 40.664 | 64.844 | 69.981 | 1.00 | 62.66 | C |
| ATOM | 2709 | OD1 | ASP | A | 366 | 40.371 | 65.573 | 70.954 | 1.00 | 64.24 | O |
| ATOM | 2710 | OD2 | ASP | A | 366 | 40.366 | 65.104 | 68.792 | 1.00 | 60.73 | O |
| ATOM | 2711 | C | ASP | A | 366 | 43.305 | 62.416 | 71.342 | 1.00 | 55.88 | C |
| ATOM | 2712 | O | ASP | A | 366 | 42.716 | 61.577 | 72.016 | 1.00 | 54.05 | O |
| ATOM | 2713 | N | SER | A | 367 | 44.499 | 62.201 | 70.805 | 1.00 | 52.78 | N |
| ATOM | 2714 | CA | SER | A | 367 | 45.222 | 60.950 | 70.987 | 1.00 | 55.09 | C |
| ATOM | 2715 | CB | SER | A | 367 | 46.170 | 60.708 | 69.810 | 1.00 | 55.82 | C |
| ATOM | 2716 | OG | SER | A | 367 | 45.448 | 60.665 | 68.591 | 1.00 | 57.99 | O |
| ATOM | 2717 | C | SER | A | 367 | 46.020 | 60.964 | 72.280 | 1.00 | 53.48 | C |
| ATOM | 2718 | O | SER | A | 367 | 47.173 | 61.370 | 72.307 | 1.00 | 53.33 | O |
| ATOM | 2719 | N | VAL | A | 368 | 45.385 | 60.521 | 73.354 | 1.00 | 50.78 | N |
| ATOM | 2720 | CA | VAL | A | 368 | 46.022 | 60.455 | 74.665 | 1.00 | 51.89 | C |
| ATOM | 2721 | CB | VAL | A | 368 | 45.386 | 61.449 | 75.652 | 1.00 | 51.57 | C |
| ATOM | 2722 | CG1 | VAL | A | 368 | 46.143 | 61.433 | 76.954 | 1.00 | 52.58 | C |
| ATOM | 2723 | CG2 | VAL | A | 368 | 45.368 | 62.837 | 75.043 | 1.00 | 53.32 | C |
| ATOM | 2724 | C | VAL | A | 368 | 45.871 | 59.048 | 75.246 | 1.00 | 51.98 | C |
| ATOM | 2725 | O | VAL | A | 368 | 44.819 | 58.413 | 75.098 | 1.00 | 52.17 | O |
| ATOM | 2726 | N | GLU | A | 369 | 46.928 | 58.579 | 75.904 | 1.00 | 50.76 | N |
| ATOM | 2727 | CA | GLU | A | 369 | 46.955 | 57.256 | 76.529 | 1.00 | 49.93 | C |
| ATOM | 2728 | CB | GLU | A | 369 | 48.250 | 57.057 | 77.338 | 1.00 | 48.09 | C |
| ATOM | 2729 | CG | GLU | A | 369 | 49.560 | 56.914 | 76.573 | 1.00 | 46.59 | C |
| ATOM | 2730 | CD | GLU | A | 369 | 50.748 | 56.640 | 77.512 | 1.00 | 54.62 | C |
| ATOM | 2731 | OE1 | GLU | A | 369 | 51.094 | 57.538 | 78.309 | 1.00 | 48.02 | O |
| ATOM | 2732 | OE2 | GLU | A | 369 | 51.343 | 55.535 | 77.459 | 1.00 | 49.79 | O |
| ATOM | 2733 | C | GLU | A | 369 | 45.773 | 57.043 | 77.484 | 1.00 | 50.07 | C |
| ATOM | 2734 | O | GLU | A | 369 | 45.438 | 57.926 | 78.268 | 1.00 | 48.36 | O |
| ATOM | 2735 | N | ARG | A | 370 | 45.149 | 55.869 | 77.393 | 1.00 | 47.33 | N |
| ATOM | 2736 | CA | ARG | A | 370 | 44.046 | 55.485 | 78.259 | 1.00 | 50.61 | C |
| ATOM | 2737 | CB | ARG | A | 370 | 42.910 | 54.826 | 77.453 | 1.00 | 52.23 | C |
| ATOM | 2738 | CG | ARG | A | 370 | 41.703 | 54.467 | 78.307 | 1.00 | 53.30 | C |
| ATOM | 2739 | CD | ARG | A | 370 | 40.472 | 54.146 | 77.465 | 1.00 | 62.56 | C |
| ATOM | 2740 | NE | ARG | A | 370 | 39.406 | 53.579 | 78.293 | 1.00 | 73.12 | N |
| ATOM | 2741 | CZ | ARG | A | 370 | 38.238 | 53.143 | 77.827 | 1.00 | 78.29 | C |
| ATOM | 2742 | NH1 | ARG | A | 370 | 37.968 | 53.211 | 76.528 | 1.00 | 82.62 | N |
| ATOM | 2743 | NH2 | ARG | A | 370 | 37.349 | 52.619 | 78.660 | 1.00 | 77.54 | N |
| ATOM | 2744 | C | ARG | A | 370 | 44.699 | 54.473 | 79.223 | 1.00 | 53.23 | C |
| ATOM | 2745 | O | ARG | A | 370 | 45.099 | 53.387 | 78.823 | 1.00 | 51.05 | O |
| ATOM | 2746 | N | LEU | A | 371 | 44.812 | 54.835 | 80.497 | 1.00 | 53.02 | N |
| ATOM | 2747 | CA | LEU | A | 371 | 45.464 | 53.974 | 81.481 | 1.00 | 53.62 | C |
| ATOM | 2748 | CB | LEU | A | 371 | 46.739 | 54.656 | 82.013 | 1.00 | 44.66 | C |
| ATOM | 2749 | CG | LEU | A | 371 | 47.733 | 55.306 | 81.044 | 1.00 | 49.67 | C |
| ATOM | 2750 | CD1 | LEU | A | 371 | 48.742 | 56.157 | 81.823 | 1.00 | 50.22 | C |
| ATOM | 2751 | CD2 | LEU | A | 371 | 48.435 | 54.232 | 80.244 | 1.00 | 46.30 | C |
| ATOM | 2752 | C | LEU | A | 371 | 44.588 | 53.603 | 82.666 | 1.00 | 57.24 | C |
| ATOM | 2753 | O | LEU | A | 371 | 43.947 | 54.456 | 83.254 | 1.00 | 57.47 | O |
| ATOM | 2754 | N | PRO | A | 372 | 44.555 | 52.311 | 83.034 | 1.00 | 63.41 | N |
| ATOM | 2755 | CD | PRO | A | 372 | 45.179 | 51.159 | 82.357 | 1.00 | 64.69 | C |
| ATOM | 2756 | CA | PRO | A | 372 | 43.750 | 51.856 | 84.173 | 1.00 | 69.47 | C |
| ATOM | 2757 | CB | PRO | A | 372 | 43.705 | 50.337 | 83.976 | 1.00 | 67.21 | C |
| ATOM | 2758 | CG | PRO | A | 372 | 45.050 | 50.046 | 83.421 | 1.00 | 66.05 | C |
| ATOM | 2759 | C | PRO | A | 372 | 44.461 | 52.255 | 85.472 | 1.00 | 74.42 | C |
| ATOM | 2760 | O | PRO | A | 372 | 45.683 | 52.162 | 85.560 | 1.00 | 71.81 | O |
| ATOM | 2761 | N | VAL | A | 373 | 43.708 | 52.702 | 86.471 | 1.00 | 82.28 | N |
| ATOM | 2762 | CA | VAL | A | 373 | 44.317 | 53.115 | 87.734 | 1.00 | 91.54 | C |
| ATOM | 2763 | CB | VAL | A | 373 | 44.187 | 54.647 | 87.935 | 1.00 | 91.16 | C |
| ATOM | 2764 | CG1 | VAL | A | 373 | 44.758 | 55.052 | 89.288 | 1.00 | 90.50 | C |
| ATOM | 2765 | CG2 | VAL | A | 373 | 44.916 | 55.379 | 86.825 | 1.00 | 90.17 | C |
| ATOM | 2766 | C | VAL | A | 373 | 43.725 | 52.420 | 88.953 | 1.00 | 98.34 | C |
| ATOM | 2767 | O | VAL | A | 373 | 42.533 | 52.112 | 88.986 | 1.00 | 97.64 | O |
| ATOM | 2768 | N | TYR | A | 374 | 44.570 | 52.181 | 89.954 | 1.00 | 108.01 | N |
| ATOM | 2769 | CA | TYR | A | 374 | 44.139 | 51.543 | 91.194 | 1.00 | 118.56 | C |
| ATOM | 2770 | CB | TYR | A | 374 | 45.312 | 50.803 | 91.849 | 1.00 | 119.70 | C |
| ATOM | 2771 | CG | TYR | A | 374 | 44.995 | 50.273 | 93.230 | 1.00 | 122.03 | C |
| ATOM | 2772 | CD1 | TYR | A | 374 | 43.941 | 49.382 | 93.429 | 1.00 | 123.81 | C |
| ATOM | 2773 | CE1 | TYR | A | 374 | 43.620 | 48.913 | 94.701 | 1.00 | 125.13 | C |
| ATOM | 2774 | CD2 | TYR | A | 374 | 45.728 | 50.684 | 94.343 | 1.00 | 121.88 | C |
| ATOM | 2775 | CE2 | TYR | A | 374 | 45.417 | 50.220 | 95.623 | 1.00 | 123.54 | C |
| ATOM | 2776 | CZ | TYR | A | 374 | 44.361 | 49.334 | 95.795 | 1.00 | 124.68 | C |
| ATOM | 2777 | OH | TYR | A | 374 | 44.044 | 48.867 | 97.055 | 1.00 | 123.74 | O |
| ATOM | 2778 | C | TYR | A | 374 | 43.563 | 52.565 | 92.183 | 1.00 | 124.31 | C |
| ATOM | 2779 | O | TYR | A | 374 | 44.268 | 53.465 | 92.648 | 1.00 | 123.92 | O |
| ATOM | 2780 | N | ASN | A | 375 | 42.279 | 52.419 | 92.497 | 1.00 | 131.24 | N |
| ATOM | 2781 | CA | ASN | A | 375 | 41.605 | 53.318 | 93.432 | 1.00 | 138.84 | C |
| ATOM | 2782 | CB | ASN | A | 375 | 40.876 | 54.436 | 92.679 | 1.00 | 139.39 | C |
| ATOM | 2783 | CG | ASN | A | 375 | 41.812 | 55.302 | 91.868 | 1.00 | 140.21 | C |
| ATOM | 2784 | OD1 | ASN | A | 375 | 42.729 | 55.920 | 92.407 | 1.00 | 141.22 | O |
| ATOM | 2785 | ND2 | ASN | A | 375 | 41.584 | 55.355 | 90.560 | 1.00 | 139.27 | N |
| ATOM | 2786 | C | ASN | A | 375 | 40.582 | 52.527 | 94.226 | 1.00 | 143.77 | C |
| ATOM | 2787 | O | ASN | A | 375 | 40.496 | 51.302 | 94.099 | 1.00 | 144.93 | O |
| ATOM | 2788 | N | LYS | A | 376 | 39.813 | 53.231 | 95.052 | 1.00 | 149.10 | N |
| ATOM | 2789 | CA | LYS | A | 376 | 38.761 | 52.592 | 95.829 | 1.00 | 154.21 | C |
| ATOM | 2790 | CB | LYS | A | 376 | 38.186 | 53.567 | 96.862 | 1.00 | 154.05 | C |
| ATOM | 2791 | CG | LYS | A | 376 | 36.779 | 53.216 | 97.339 | 1.00 | 154.47 | C |
| ATOM | 2792 | CD | LYS | A | 376 | 36.712 | 51.817 | 97.932 | 1.00 | 155.03 | C |
| ATOM | 2793 | CE | LYS | A | 376 | 35.269 | 51.383 | 98.147 | 1.00 | 155.25 | C |
| ATOM | 2794 | NZ | LYS | A | 376 | 34.539 | 52.317 | 99.044 | 1.00 | 155.35 | N |
| ATOM | 2795 | C | LYS | A | 376 | 37.686 | 52.206 | 94.817 | 1.00 | 157.92 | C |
| ATOM | 2796 | O | LYS | A | 376 | 36.869 | 51.316 | 95.059 | 1.00 | 158.76 | O |
| ATOM | 2797 | N | SER | A | 377 | 37.716 | 52.888 | 93.674 | 1.00 | 161.94 | N |
| ATOM | 2798 | CA | SER | A | 377 | 36.773 | 52.659 | 92.581 | 1.00 | 165.54 | C |
| ATOM | 2799 | CB | SER | A | 377 | 36.880 | 53.791 | 91.551 | 1.00 | 165.08 | C |
| ATOM | 2800 | OG | SER | A | 377 | 36.053 | 53.540 | 90.427 | 1.00 | 164.33 | O |
| ATOM | 2801 | C | SER | A | 377 | 37.031 | 51.323 | 91.892 | 1.00 | 168.07 | C |
| ATOM | 2802 | O | SER | A | 377 | 36.099 | 50.568 | 91.613 | 1.00 | 168.27 | O |
| ATOM | 2803 | N | ALA | A | 378 | 38.301 | 51.046 | 91.613 | 1.00 | 170.96 | N |
| ATOM | 2804 | CA | ALA | A | 378 | 38.700 | 49.807 | 90.957 | 1.00 | 173.55 | C |
| ATOM | 2805 | CB | ALA | A | 378 | 40.123 | 49.935 | 90.433 | 1.00 | 173.23 | C |
| ATOM | 2806 | C | ALA | A | 378 | 38.603 | 48.626 | 91.916 | 1.00 | 175.43 | C |
| ATOM | 2807 | O | ALA | A | 378 | 38.577 | 47.469 | 91.493 | 1.00 | 175.80 | O |
| ATOM | 2808 | N | TRP | A | 379 | 38.547 | 48.926 | 93.209 | 1.00 | 177.50 | N |
| ATOM | 2809 | CA | TRP | A | 379 | 38.462 | 47.897 | 94.238 | 1.00 | 179.78 | C |
| ATOM | 2810 | CB | TRP | A | 379 | 38.721 | 48.513 | 95.614 | 1.00 | 180.61 | C |
| ATOM | 2811 | CG | TRP | A | 379 | 38.689 | 47.511 | 96.720 | 1.00 | 182.46 | C |
| ATOM | 2812 | CD2 | TRP | A | 379 | 37.556 | 47.164 | 97.527 | 1.00 | 183.67 | C |
| ATOM | 2813 | CE2 | TRP | A | 379 | 37.967 | 46.133 | 98.400 | 1.00 | 183.99 | C |
| ATOM | 2814 | CE3 | TRP | A | 379 | 36.235 | 47.628 | 97.600 | 1.00 | 183.99 | C |
| ATOM | 2815 | CD1 | TRP | A | 379 | 39.709 | 46.700 | 97.124 | 1.00 | 182.63 | C |
| ATOM | 2816 | NE1 | TRP | A | 379 | 39.284 | 45.868 | 98.131 | 1.00 | 183.47 | N |
| ATOM | 2817 | CZ2 | TRP | A | 379 | 37.099 | 45.549 | 99.335 | 1.00 | 184.20 | C |
| ATOM | 2818 | CZ3 | TRP | A | 379 | 35.372 | 47.049 | 98.530 | 1.00 | 184.22 | C |
| ATOM | 2819 | CH2 | TRP | A | 379 | 35.811 | 46.022 | 99.386 | 1.00 | 184.39 | C |
| ATOM | 2820 | C | TRP | A | 379 | 37.114 | 47.173 | 94.262 | 1.00 | 181.09 | C |
| ATOM | 2821 | O | TRP | A | 379 | 37.063 | 45.944 | 94.251 | 1.00 | 181.24 | O |
| ATOM | 2822 | N | ARG | A | 380 | 36.027 | 47.942 | 94.294 | 1.00 | 182.45 | N |
| ATOM | 2823 | CA | ARG | A | 380 | 34.676 | 47.383 | 94.351 | 1.00 | 183.58 | C |
| ATOM | 2824 | CB | ARG | A | 380 | 33.701 | 48.456 | 94.848 | 1.00 | 182.80 | C |
| ATOM | 2825 | CG | ARG | A | 380 | 33.806 | 49.778 | 94.115 | 1.00 | 182.38 | C |
| ATOM | 2826 | CD | ARG | A | 380 | 33.118 | 50.884 | 94.892 | 1.00 | 182.15 | C |
| ATOM | 2827 | NE | ARG | A | 380 | 33.343 | 52.192 | 94.284 | 1.00 | 182.11 | N |
| ATOM | 2828 | CZ | ARG | A | 380 | 32.991 | 53.346 | 94.842 | 1.00 | 181.83 | C |
| ATOM | 2829 | NH1 | ARG | A | 380 | 32.396 | 53.357 | 96.027 | 1.00 | 181.82 | N |
| ATOM | 2830 | NH2 | ARG | A | 380 | 33.239 | 54.490 | 94.219 | 1.00 | 181.70 | N |
| ATOM | 2831 | C | ARG | A | 380 | 34.130 | 46.734 | 93.075 | 1.00 | 184.68 | C |
| ATOM | 2832 | O | ARG | A | 380 | 32.966 | 46.329 | 93.038 | 1.00 | 184.74 | O |
| ATOM | 2833 | N | HIS | A | 381 | 34.955 | 46.632 | 92.034 | 1.00 | 185.93 | N |
| ATOM | 2834 | CA | HIS | A | 381 | 34.527 | 45.993 | 90.788 | 1.00 | 186.80 | C |
| ATOM | 2835 | CB | HIS | A | 381 | 35.277 | 46.568 | 89.579 | 1.00 | 186.21 | C |
| ATOM | 2836 | CG | HIS | A | 381 | 34.741 | 47.881 | 89.094 | 1.00 | 185.83 | C |
| ATOM | 2837 | CD2 | HIS | A | 381 | 34.121 | 48.216 | 87.938 | 1.00 | 185.49 | C |
| ATOM | 2838 | ND1 | HIS | A | 381 | 34.827 | 49.042 | 89.832 | 1.00 | 185.55 | N |
| ATOM | 2839 | CE1 | HIS | A | 381 | 34.284 | 50.036 | 89.151 | 1.00 | 185.22 | C |
| ATOM | 2840 | NE2 | HIS | A | 381 | 33.849 | 49.561 | 87.997 | 1.00 | 185.15 | N |
| ATOM | 2841 | C | HIS | A | 381 | 34.808 | 44.495 | 90.895 | 1.00 | 187.59 | C |
| ATOM | 2842 | O | HIS | A | 381 | 34.431 | 43.715 | 90.019 | 1.00 | 187.77 | O |
| ATOM | 2843 | N | TYR | A | 382 | 35.477 | 44.110 | 91.980 | 1.00 | 188.40 | N |
| ATOM | 2844 | CA | TYR | A | 382 | 35.820 | 42.716 | 92.248 | 1.00 | 188.93 | C |
| ATOM | 2845 | CB | TYR | A | 382 | 37.288 | 42.606 | 92.670 | 1.00 | 189.39 | C |
| ATOM | 2846 | CG | TYR | A | 382 | 38.280 | 42.933 | 91.575 | 1.00 | 189.52 | C |
| ATOM | 2847 | CD1 | TYR | A | 382 | 38.393 | 42.120 | 90.446 | 1.00 | 189.20 | C |
| ATOM | 2848 | CE1 | TYR | A | 382 | 39.309 | 42.412 | 89.441 | 1.00 | 188.85 | C |
| ATOM | 2849 | CD2 | TYR | A | 382 | 39.111 | 44.050 | 91.670 | 1.00 | 189.30 | C |
| ATOM | 2850 | CE2 | TYR | A | 382 | 40.029 | 44.352 | 90.672 | 1.00 | 189.01 | C |
| ATOM | 2851 | CZ | TYR | A | 382 | 40.123 | 43.529 | 89.561 | 1.00 | 188.91 | C |
| ATOM | 2852 | OH | TYR | A | 382 | 41.027 | 43.824 | 88.569 | 1.00 | 188.60 | O |
| ATOM | 2853 | C | TYR | A | 382 | 34.933 | 42.144 | 93.354 | 1.00 | 188.96 | C |
| ATOM | 2854 | O | TYR | A | 382 | 35.086 | 40.987 | 93.749 | 1.00 | 188.67 | O |
| ATOM | 2855 | N | GLN | A | 383 | 34.007 | 42.962 | 93.850 | 1.00 | 189.10 | N |
| ATOM | 2856 | CA | GLN | A | 383 | 33.101 | 42.535 | 94.912 | 1.00 | 189.14 | C |
| ATOM | 2857 | CB | GLN | A | 383 | 32.748 | 43.720 | 95.822 | 1.00 | 188.28 | C |
| ATOM | 2858 | CG | GLN | A | 383 | 33.942 | 44.429 | 96.454 | 1.00 | 187.50 | C |
| ATOM | 2859 | CD | GLN | A | 383 | 34.963 | 43.468 | 97.031 | 1.00 | 187.29 | C |
| ATOM | 2860 | OE1 | GLN | A | 383 | 35.784 | 42.908 | 96.303 | 1.00 | 187.30 | O |
| ATOM | 2861 | NE2 | GLN | A | 383 | 34.910 | 43.262 | 98.341 | 1.00 | 186.67 | N |
| ATOM | 2862 | C | GLN | A | 383 | 31.818 | 41.940 | 94.334 | 1.00 | 189.41 | C |
| ATOM | 2863 | O | GLN | A | 383 | 30.742 | 42.178 | 94.922 | 1.00 | 189.86 | O |
| ATOM | 2864 | OXT | GLN | A | 383 | 31.905 | 41.225 | 93.315 | 1.00 | 189.30 | O |
| TER | 2865 | | GLN | A | 383 | | | | | | |
| ATOM | 2866 | P2 | ADP | B | 500 | 40.557 | 32.927 | 66.822 | 1.00 | 112.47 | P |
| ATOM | 2867 | O4 | ADP | B | 500 | 39.721 | 31.744 | 67.113 | 1.00 | 111.94 | O |
| ATOM | 2868 | O5 | ADP | B | 500 | 40.322 | 33.445 | 65.473 | 1.00 | 113.24 | O |
| ATOM | 2869 | O6 | ADP | B | 500 | 42.098 | 32.507 | 66.948 | 1.00 | 112.75 | O |
| ATOM | 2870 | P3 | ADP | B | 500 | 39.656 | 35.569 | 67.981 | 1.00 | 102.56 | P |
| ATOM | 2871 | O7 | ADP | B | 500 | 40.092 | 36.301 | 66.821 | 1.00 | 101.78 | O |
| ATOM | 2872 | O8 | ADP | B | 500 | 39.804 | 36.183 | 69.319 | 1.00 | 100.98 | O |
| ATOM | 2873 | O9 | ADP | B | 500 | 40.308 | 34.076 | 67.909 | 1.00 | 109.08 | O |
| ATOM | 2874 | O10 | ADP | B | 500 | 38.071 | 35.439 | 67.986 | 1.00 | 103.89 | O |
| ATOM | 2875 | C1 | ADP | B | 500 | 37.533 | 34.580 | 67.003 | 1.00 | 101.07 | C |
| ATOM | 2876 | C3 | ADP | B | 500 | 36.190 | 33.963 | 67.020 | 1.00 | 98.50 | C |
| ATOM | 2877 | O11 | ADP | B | 500 | 35.065 | 34.759 | 66.572 | 1.00 | 96.97 | O |
| ATOM | 2878 | C7 | ADP | B | 500 | 36.418 | 32.886 | 65.946 | 1.00 | 97.00 | C |
| ATOM | 2879 | O12 | ADP | B | 500 | 36.878 | 31.670 | 66.573 | 1.00 | 95.63 | O |
| ATOM | 2880 | C9 | ADP | B | 500 | 35.137 | 32.812 | 65.117 | 1.00 | 95.75 | C |
| ATOM | 2881 | O13 | ADP | B | 500 | 34.369 | 31.735 | 65.593 | 1.00 | 94.39 | O |
| ATOM | 2882 | C10 | ADP | B | 500 | 34.492 | 34.212 | 65.398 | 1.00 | 93.90 | C |
| ATOM | 2883 | N2 | ADP | B | 500 | 34.659 | 35.317 | 64.372 | 1.00 | 88.10 | N |
| ATOM | 2884 | C8 | ADP | B | 500 | 35.520 | 36.407 | 64.340 | 1.00 | 85.59 | C |
| ATOM | 2885 | N4 | ADP | B | 500 | 35.435 | 37.168 | 63.305 | 1.00 | 83.42 | N |
| ATOM | 2886 | C5 | ADP | B | 500 | 34.452 | 36.571 | 62.555 | 1.00 | 82.67 | C |
| ATOM | 2887 | C6 | ADP | B | 500 | 33.839 | 36.856 | 61.262 | 1.00 | 80.47 | C |
| ATOM | 2888 | N5 | ADP | B | 500 | 34.222 | 37.898 | 60.539 | 1.00 | 74.22 | N |
| ATOM | 2889 | N1 | ADP | B | 500 | 32.827 | 36.021 | 60.771 | 1.00 | 80.27 | N |
| ATOM | 2890 | C2 | ADP | B | 500 | 32.409 | 34.923 | 61.504 | 1.00 | 79.97 | C |
| ATOM | 2891 | N3 | ADP | B | 500 | 32.932 | 34.573 | 62.721 | 1.00 | 81.76 | N |
| ATOM | 2892 | C4 | ADP | B | 500 | 33.940 | 35.415 | 63.187 | 1.00 | 83.79 | C |
| TER | 2893 | | ADP | B | 500 | | | | | | |
| ATOM | 2894 | MG + 2 | MG2 | C | 501 | 41.894 | 35.428 | 64.890 | 1.00 | 82.23 | MG |
| TER | 2895 | | MG2 | C | 501 | | | | | | |
| ATOM | 2896 | O | HOH | W | 1 | 32.989 | 57.367 | 63.342 | 1.00 | 23.04 | O |
| ATOM | 2897 | O | HOH | W | 2 | 60.152 | 58.403 | 76.432 | 1.00 | 45.64 | O |
| ATOM | 2898 | O | HOH | W | 3 | 40.385 | 63.400 | 66.713 | 1.00 | 36.36 | O |
| ATOM | 2899 | O | HOH | W | 4 | 47.018 | 51.773 | 69.811 | 1.00 | 32.39 | O |
| ATOM | 2900 | O | HOH | W | 5 | 29.418 | 54.281 | 65.658 | 1.00 | 36.06 | O |
| ATOM | 2901 | O | HOH | W | 6 | 30.737 | 56.478 | 64.913 | 1.00 | 24.80 | O |
| ATOM | 2902 | O | HOH | W | 7 | 43.953 | 54.100 | 69.312 | 1.00 | 33.91 | O |
| ATOM | 2903 | O | HOH | W | 8 | 31.431 | 44.776 | 69.271 | 1.00 | 32.10 | O |
| ATOM | 2904 | O | HOH | W | 9 | 48.862 | 28.035 | 87.080 | 1.00 | 53.27 | O |
| ATOM | 2905 | O | HOH | W | 10 | 35.113 | 41.516 | 58.615 | 1.00 | 37.71 | O |
| ATOM | 2906 | O | HOH | W | 11 | 35.411 | 22.757 | 91.765 | 1.00 | 54.10 | O |
| ATOM | 2907 | O | HOH | W | 12 | 33.663 | 46.196 | 71.017 | 1.00 | 43.05 | O |
| ATOM | 2908 | O | HOH | W | 13 | 35.407 | 58.632 | 60.280 | 1.00 | 34.43 | O |
| ATOM | 2909 | O | HOH | W | 14 | 52.852 | 58.961 | 87.174 | 1.00 | 39.78 | O |
| ATOM | 2910 | O | HOH | W | 15 | 36.688 | 39.705 | 63.256 | 1.00 | 35.46 | O |
| ATOM | 2911 | O | HOH | W | 16 | 37.134 | 57.808 | 67.302 | 1.00 | 42.49 | O |
| ATOM | 2912 | O | HOH | W | 17 | 38.904 | 22.288 | 93.371 | 1.00 | 42.27 | O |
| ATOM | 2913 | O | HOH | W | 18 | 39.154 | 43.794 | 74.929 | 1.00 | 42.25 | O |
| ATOM | 2914 | O | HOH | W | 19 | 69.319 | 56.280 | 78.119 | 1.00 | 52.66 | O |
| ATOM | 2915 | O | HOH | W | 20 | 43.490 | 33.783 | 85.900 | 1.00 | 51.48 | O |
| ATOM | 2916 | O | HOH | W | 21 | 39.766 | 48.658 | 79.593 | 1.00 | 62.72 | O |
| ATOM | 2917 | O | HOH | W | 22 | 45.024 | 55.756 | 53.771 | 1.00 | 47.81 | O |
| ATOM | 2918 | O | HOH | W | 23 | 28.308 | 51.843 | 59.582 | 1.00 | 34.63 | O |
| ATOM | 2919 | O | HOH | W | 24 | 33.970 | 37.943 | 52.163 | 1.00 | 46.26 | O |
| ATOM | 2920 | O | HOH | W | 25 | 46.675 | 62.271 | 63.917 | 1.00 | 44.23 | O |
| ATOM | 2921 | O | HOH | W | 26 | 36.883 | 36.300 | 91.018 | 1.00 | 61.15 | O |
| ATOM | 2922 | O | HOH | W | 27 | 32.879 | 42.650 | 76.173 | 1.00 | 56.23 | O |
| ATOM | 2923 | O | HOH | W | 28 | 52.544 | 64.988 | 60.426 | 1.00 | 48.25 | O |
| ATOM | 2924 | O | HOH | W | 29 | 61.645 | 54.104 | 98.113 | 1.00 | 65.30 | O |
| ATOM | 2925 | O | HOH | W | 30 | 34.777 | 70.369 | 63.481 | 1.00 | 47.49 | O |
| ATOM | 2926 | O | HOH | W | 31 | 44.808 | 58.129 | 67.253 | 1.00 | 39.83 | O |
| ATOM | 2927 | O | HOH | W | 32 | 52.958 | 37.963 | 76.119 | 1.00 | 42.86 | O |
| ATOM | 2928 | O | HOH | W | 33 | 28.519 | 52.321 | 67.667 | 1.00 | 38.04 | O |
| ATOM | 2929 | O | HOH | W | 34 | 34.228 | 60.366 | 68.522 | 1.00 | 50.27 | O |
| ATOM | 2930 | O | HOH | W | 35 | 36.663 | 36.260 | 80.308 | 1.00 | 47.39 | O |
| ATOM | 2931 | O | HOH | W | 36 | 58.729 | 59.321 | 71.829 | 1.00 | 47.99 | O |
| ATOM | 2932 | O | HOH | W | 37 | 39.672 | 32.973 | 62.861 | 1.00 | 56.11 | O |
| ATOM | 2933 | O | HOH | W | 38 | 58.436 | 35.897 | 78.980 | 1.00 | 55.56 | O |
| ATOM | 2934 | O | HOH | W | 39 | 45.392 | 25.343 | 88.713 | 1.00 | 63.21 | O |
| ATOM | 2935 | O | HOH | W | 40 | 38.469 | 35.546 | 71.455 | 1.00 | 72.71 | O |
| ATOM | 2936 | O | HOH | W | 41 | 49.659 | 62.630 | 59.007 | 1.00 | 56.89 | O |
| ATOM | 2937 | O | HOH | W | 42 | 37.641 | 37.251 | 46.390 | 1.00 | 58.22 | O |
| ATOM | 2938 | O | HOH | W | 43 | 69.429 | 63.294 | 70.946 | 1.00 | 62.58 | O |
| ATOM | 2939 | O | HOH | W | 44 | 32.835 | 46.987 | 42.918 | 1.00 | 54.09 | O |
| ATOM | 2940 | O | HOH | W | 45 | 56.765 | 59.368 | 57.580 | 1.00 | 52.18 | O |
| ATOM | 2941 | O | HOH | W | 46 | 48.769 | 65.554 | 71.422 | 1.00 | 65.09 | O |
| ATOM | 2942 | O | HOH | W | 47 | 36.200 | 54.609 | 73.760 | 1.00 | 67.23 | O |
| ATOM | 2943 | O | HOH | W | 48 | 46.962 | 60.743 | 66.231 | 1.00 | 54.48 | O |
| ATOM | 2944 | O | HOH | W | 49 | 26.017 | 53.617 | 56.300 | 1.00 | 46.92 | O |
| ATOM | 2945 | O | HOH | W | 50 | 36.221 | 65.425 | 72.969 | 1.00 | 67.76 | O |
| ATOM | 2946 | O | HOH | W | 51 | 43.858 | 57.686 | 82.181 | 1.00 | 54.77 | O |
| ATOM | 2947 | O | HOH | W | 52 | 36.531 | 33.671 | 61.585 | 1.00 | 60.86 | O |
| ATOM | 2948 | O | HOH | W | 53 | 33.417 | 48.501 | 73.109 | 1.00 | 62.84 | O |
| ATOM | 2949 | O | HOH | W | 54 | 37.342 | 64.721 | 56.542 | 1.00 | 46.52 | O |
| ATOM | 2950 | O | HOH | W | 55 | 27.587 | 34.934 | 49.149 | 1.00 | 66.64 | O |
| ATOM | 2951 | O | HOH | W | 56 | 26.727 | 38.611 | 48.701 | 1.00 | 73.46 | O |
| ATOM | 2952 | O | HOH | W | 57 | 30.948 | 36.557 | 58.807 | 1.00 | 30.35 | O |
| ATOM | 2953 | O | HOH | W | 58 | 42.952 | 56.541 | 68.541 | 1.00 | 45.48 | O |
| ATOM | 2954 | O | HOH | W | 59 | 36.897 | 40.576 | 60.771 | 1.00 | 35.37 | O |
| ATOM | 2955 | O | HOH | W | 60 | 27.639 | 35.803 | 73.843 | 1.00 | 55.77 | O |
| ATOM | 2956 | O | HOH | W | 61 | 26.191 | 39.045 | 72.082 | 1.00 | 50.74 | O |
| ATOM | 2957 | O | HOH | W | 62 | 58.848 | 65.166 | 66.496 | 1.00 | 63.31 | O |
| ATOM | 2958 | O | HOH | W | 63 | 40.543 | 62.448 | 73.427 | 1.00 | 52.55 | O |
| ATOM | 2959 | O | HOH | W | 64 | 35.223 | 31.490 | 61.532 | 1.00 | 59.42 | O |
| ATOM | 2960 | O | HOH | W | 65 | 48.450 | 28.966 | 56.562 | 1.00 | 54.96 | O |
| ATOM | 2961 | O | HOH | W | 66 | 43.404 | 52.960 | 48.444 | 1.00 | 56.71 | O |
| ATOM | 2962 | O | HOH | W | 67 | 47.351 | 50.309 | 87.381 | 1.00 | 49.52 | O |
| ATOM | 2963 | O | HOH | W | 68 | 28.522 | 53.994 | 57.746 | 1.00 | 44.67 | O |
| ATOM | 2964 | O | HOH | W | 69 | 31.323 | 31.918 | 65.890 | 1.00 | 79.54 | O |
| ATOM | 2965 | O | HOH | W | 70 | 66.859 | 63.113 | 70.464 | 1.00 | 72.79 | O |
| ATOM | 2966 | O | HOH | W | 71 | 33.474 | 48.407 | 54.123 | 1.00 | 39.20 | O |
| ATOM | 2967 | O | HOH | W | 72 | 49.912 | 52.015 | 83.104 | 1.00 | 48.98 | O |
| ATOM | 2968 | O | HOH | W | 73 | 33.131 | 64.850 | 59.306 | 1.00 | 45.65 | O |
| ATOM | 2969 | O | HOH | W | 74 | 45.852 | 33.625 | 85.005 | 1.00 | 68.31 | O |
| ATOM | 2970 | O | HOH | W | 75 | 35.326 | 56.208 | 46.954 | 1.00 | 67.08 | O |
| ATOM | 2971 | O | HOH | W | 76 | 61.425 | 58.924 | 73.591 | 1.00 | 56.54 | O |
| ATOM | 2972 | O | HOH | W | 77 | 40.143 | 65.286 | 60.148 | 1.00 | 42.24 | O |
| ATOM | 2973 | O | HOH | W | 78 | 50.541 | 61.079 | 91.342 | 1.00 | 62.51 | O |
| ATOM | 2974 | O | HOH | W | 79 | 26.219 | 49.913 | 69.839 | 1.00 | 59.22 | O |
| ATOM | 2975 | O | HOH | W | 80 | 30.726 | 32.449 | 59.929 | 1.00 | 65.02 | O |
| ATOM | 2976 | O | HOH | W | 81 | 26.445 | 35.397 | 51.917 | 1.00 | 61.09 | O |
| ATOM | 2977 | O | HOH | W | 82 | 35.851 | 43.578 | 77.610 | 1.00 | 61.66 | O |
| ATOM | 2978 | O | HOH | W | 83 | 40.228 | 66.076 | 55.296 | 1.00 | 53.40 | O |
| ATOM | 2979 | O | HOH | W | 84 | 52.893 | 45.650 | 68.981 | 1.00 | 64.69 | O |
| ATOM | 2980 | O | HOH | W | 85 | 61.579 | 40.888 | 103.314 | 1.00 | 62.65 | O |
| ATOM | 2981 | O | HOH | W | 86 | 50.134 | 23.816 | 92.589 | 1.00 | 63.77 | O |
| ATOM | 2982 | O | HOH | W | 87 | 40.795 | 49.195 | 45.579 | 1.00 | 58.11 | O |
| ATOM | 2983 | O | HOH | W | 88 | 38.800 | 55.131 | 47.133 | 1.00 | 76.42 | O |

1. A PDHK crystal wherein the PDHK protein comprises PDHK-2 amino acid sequence as shown in SEQ ID NO: 2 but starting at amino acid position 16, or a homologue, fragment, variant or derivative thereof.

2. A PDHK crystal according to claim 1, wherein the PDHK-2 protein consists of PDHK-2 amino acid sequence as shown in SEQ ID NO: 2, but starting at amino acid position 16, or a homologue, fragment, variant or derivative thereof.

3. A PDHK crystal according to claim 1, wherein the PDHK-2 amino acid sequence is preceded by an amino acid sequence other than the amino acid sequence at positions 1 to 15 of SEQ ID NO: 2.

4. A PDHK crystal according to claim 3, wherein the PDHK-2 amino acid sequence is preceded by amino acid sequence comprising an affinity purification tag.

5. A PDHK sequence according to claim 3, wherein the PDHK-2 amino acid sequence is preceded by an amino acid sequence comprising a specific protein cleavage site.

6. A PDHK crystal according to claim 3, wherein the PDHK-2 amino acid sequence is preceded by an amino acid sequence comprising an affinity purification tag followed by a specific protein cleavage site.

7. A PDHK crystal according to claim 4 or claim 6, wherein the affinity purification tag comprises a multiple Histidine residue tag.

8. A PDHK crystal according to claim 7, wherein the multiple Histidine residue tag is a Hexa-His tag.

9. A PDHK crystal according to claim 5 or claim 6, wherein the specific protein cleavage site is a thrombin cleavage site.

10. A PDHK crystal according to claim 6, wherein the affinity purification tag is a Hexa-His tag and the specific protein cleavage site is a thrombin cleavage site.

11. A PDHK crystal according to claim 10, wherein the PDHK protein comprises a PDHK amino acid sequence preceded by a Hexa-His tag followed by a thrombin cleavage site.

12. A PDHK crystal according to claim 11, wherein the PDHK protein additionally comprises an N-terminal methionine amino acid residue.

13. A PDHK crystal according to claim 12, wherein the PDHK-2 amino acid sequence is as shown in SEQ ID NO: 4.

14. A PDHK crystal, wherein the PDHK protein comprises a PDHK-2 amino acid sequence as shown in SEQ ID NO: 5, or a homologue, fragment, variant or derivative thereof.

15. A PDHK crystal according to claim 1, wherein the PDHK protein consists of a PDHK-2 amino acid sequence preceded by one or two amino acid residues.

16. A PDHK crystal according to claim 16, wherein the PDHK protein consists of a PDHK amino acid sequence as shown in SEQ ID No: 5.

17. A PDHK crystal according to claim 16, wherein the PDHK-2 amino acid sequence is prepared by thrombin cleavage of the PDHK-2 amino acid sequence as shown in SEQ NO: 4.

18. A crystal of claim 14 or 17, which is grown in 100 mM sodium acetate pH 5.2-5.8, 5-10% isopropanol, and 25-150 mM MgCl₂.

19. A crystal of claim 14, which is grown in pH 5.6-5.8, 6-9% isopropanol, and 75-125 mM MgCl₂.

20. A crystal of claim 14, wherein the crystal has a space group P6₄.

21. A crystal of claim 14, wherein the crystal has unit cell dimensions of a=b=109 Å+/−3 Å, c−85 Å+/−3 Å.

22. A crystal of claim 11, wherein the crystal is monoclinic.

23. A crystal of claim 11 which is grown using 100 mM citrate pH 5.5-5.7, 15% PEG 4K, and between 200-400 mM ammonium acetate.

24. A crystal of claim 22, which is grown using 100 mM citrate pH 5.5-5.7, 15% PEG 4K, and between 200-400 mM ammonium acetate.

25. A crystal of claim 11, which has a space group C2.

26. A crystal of claim 11, which has unit cell dimensions of a=90 Å+/−3 Å, b=54 Å+/−3 Å, c=83 Å+/−3 Å, and β=106°+/−2°.

27. A crystal of claim 1, which has a heavy atom soaked in.

28. A crystal of claim 27, wherein the heavy atom is selected from mercury, iridium, and osmium.

29. A crystal of claim 1, which has a PDHK ligand and, optionally, ATP or ADP soaked in.

30. A PDHK-2 co-crystal with a PDHK ligand.

31. A crystal of claim 30, wherein the PDHK-2 amino acid sequence comprises the sequence of SEQ ID NO: 5.

32. A crystal of claim 31, wherein the crystal is grown in 100 mM MES pH6, 10% isopropanol and 200 mM calcium acetate in the presence of a PDHK ligand.

33. A crystal of claim 30, wherein the ligand is 4-{(2,5)-dimethyl-4-[3,3,3-trifluoro-2-hydroxy-2-methylpropanoyl]piperazinyl}carbonyl) benzonitrile.

34. A crystal of claim 30, wherein ATP is soaked in.

35. A crystal of claim 29, wherein the ligand is N-{4-[(ethylanilino)sulfonyl]-2-methylphenyl}-3,3,3-trifluoro-2-hydroxy-2-methylpropanamide.

36. A crystal of claim 29, wherein the ligand is N-(2-aminoethyl)-2-{3-chloro-4-[(4-isopropylbenzyl)oxy]phenyl}acetamide.

37. A crystal of claim 29, wherein the PDHK ligand is DCA and ADP is soaked in.

38. A crystal of claim 1 which diffracts X-rays to 3.8 Å or better resolution.

39. A crystal of claim 38, which diffracts X-rays to 3.2 Å or better resolution.

40. A crystal of claim 38, which diffracts X-rays to 2.8 Å or better resolution.

41. A crystal of claim 38, which diffracts X-rays to 2.5 Å or better resolution.

42. A crystal of claim 38, which diffracts X-rays to 2.4 Å or better resolution.

43. A crystal of claim 38, which diffracts X-rays to 2.2 Å or better resolution.

44. A crystal of claim 14, having the atomic coordinates set out in Table 3, or a derivative set as expressed in any reference frame.

45. A crystal of claim 30, having the atomic coordinates set out in Table 4, or a derivative set as expressed in any reference frame.

46. A crystal of claim 34, having the atomic coordinates set out in Table 5, or a derivative set as expressed in any reference frame.

47. A crystal of claim 35, having the atomic coordinates set out in Table 6, or a derivative set as expressed in any reference frame.

48. A crystal of claim 36, having the atomic coordinates set out in Table 7, or a derivative set as expressed in any reference frame.

49. A crystal of claim 37, having the atomic coordinates set out in Table 8, or a derivative set as expressed in any reference frame.

50. The use of atomic coordinates of a crystal selected from any one crystal of claim 1-49 for deriving the three-dimensional structure of PDHK-2.

51. A method for evaluating the binding interaction of a compound with PDHK-2, comprising deriving the three dimensional structure of PDHK-2 using the atomic coordinates of a crystal selected from any one crystal of claim 1-49, and evaluating the binding interaction of a compound with PDHK-2.

52. The method of claim 51, wherein said evaluation of the binding interaction comprises evaluating the binding interaction of a compound with a binding site on PDHK-2.

53. A method of claim 52, wherein said binding site is an ATP binding site comprising one or more amino acid residues selected from Glu251, Leu252, Lys254, Asn255, Ala256, Arg258, Ala259, Met288, Ser289, Asp290, Gly292, Gly293, Gly294, Val295, Ile300, Leu303, Leu323, Ala324, Gly325, Phe326, Gly327, Tyr328, Gly329, Leu330, Pro331, Leu346, Ser348, Thr354, Asp355, Ala356.

54. A method of claim 52, wherein said binding site is a putative E₂L₂ binding site comprising one or more amino acid residues selected from Leu31, Gln35, Phe36, Asp38, Phe39, Thr48, Ser49, Phe52, Leu53, Met167, Leu168, Gln171, His172, Ile175, Phe176.

55. A method of claim 52, wherein said binding site is comprises one or more amino acid residues selected from Leu71, Pro72, Arg74, Val75, Thr78, Ser80, Val81, Met130, Gly133, Val134, Glu136, Tyr137, Asp144, Val146, Ser147, Asn150, Ile151, Phe154, Leu155, Tyr382.

56. A method of claim 52, wherein said binding site is comprises one or more amino acid residues selected from Leu57, Leu61, Tyr88, Ser91, Ile95, Ile119, Arg120, His123, Ser161, Arg162, Ile165, Arg166, Ile169.

57. The use of the three-dimensional structure of PDHK-2 as derived in claim 50 to design a compound capable of associating with PDHK-2.

58. The use of the three-dimensional structure of PDHK-2 as derived in claim 50 to design a compound capable of associating with a binding site of PDHK-2.

59. The use of the three-dimensional structure of PDHK-2 as derived in claim 50 to design a compound capable of associating with a binding site of PDHK-2 selected from any one of the binding sites defined in claims 53 to 56.

60. The use according to claim 57, wherein the compound is an inhibitor of PDHK-2.

61. A method of selecting a PDHK-2 inhibitor compound from a group of potential PDHK-2 inhibitor compounds, comprising the following steps:
a) creating a three-dimensional representation of the structure of PDHK-2 as derived in claim 50;
b) displaying and superimposing a model of the potential PDHK-2 inhibitor compound on the model of the PDHK-2 structure;
c) assessing whether the model of the potential PDHK-2 inhibitor compound fits the model of the PDHK-2 structure.

62. A method of selecting a PDHK-2 inhibitor compound from a group of potential PDHK-2 inhibitor compounds, comprising the following steps:
a) creating a three-dimensional representation of any one of the binding sites of PDHK-2 by a method of claim 50, in a suitable computer program;
b) displaying and superimposing a model of the potential PDHK-2 inhibitor compound on the model of said binding site;
c) assessing whether the model of the potential PDHK-2 inhibitor compound fits the binding site model.

63. A method of claim 61 or 62, further comprising the following steps:
d) incorporating the potential PDHK-2 inhibitor compound in a biological PDHK-2 activity assay;
e) determining whether the potential PDHK-2 inhibitor compound inhibits PDHK-2 activity in this assay.

64. The use of the atomic coordinates of PDHK-2 as defined in claims 44 to 49, or portions thereof, to solve a crystal form of a mutant, homologue or co-complex of PDHK-2, for example by Molecular Replacement or Difference Fourier analysis.

65. The use of the atomic coordinates of PDHK-2 as defined in claims 44 to 49, or portions thereof, to produce a model of the three-dimensional structure of related proteins.

66. The use of the three-dimensional structure of PDHK-2 as derived in claim 50 to design site-directed mutants that mimic other PDHK isozymes or variants thereof.

67. A nucleotide sequence comprising (a) the sequence shown in SEQ ID NO: 3, or allelic variants, or mutants thereof; or (b) a sequence encoding the peptide sequence as shown in SEQ ID NO: 4 or allelic variants, or mutants thereof; or (c) the sequence as shown in SEQ ID NO: 9, or allelic variants, or mutants thereof; or (d) a sequence encoding PDHK-2 amino acid sequence as shown in SEQ ID NO: 2, but starting at amino acid position 16, or allelic variants or mutants thereof; or (e) the sequence as shown in SEQ ID NO: 8 or allelic variants, or mutants thereof; or (f) a sequence encoding the amino acid sequence shown in SEQ ID No: 5, or allelic variants or mutants thereof.

68. An expression vector, comprising a nucleotide sequence of claim 67 linked with a regulatory sequence suitable for driving expression of the PDHK protein in a host cell.

69. A recombinant baculovirus, comprising a nucleotide sequence of claim 67.
